(19) United States
(12) Patent Application Publication
(10) Pub. No.: US 2026/0247854 A1
(43) Pub. Date: Aug. 20, 2026

(54) AROMATIC AMINE COMPOUND, ELECTROLUMINESCENT DEVICE, AND DISPLAY APPARATUS

(71) Applicant: BOE TECHNOLOGY GROUP CO., LTD., Beijing (CN)

(72) Inventors: Lixia QIU, Beijing (CN); Yuqian SUN, Beijing (CN); Dan WANG, Beijing (CN); Lei CHEN, Beijing (CN); Haiyan SUN, Beijing (CN)

(73) Assignee: BOE TECHNOLOGY GROUP CO., LTD., Beijing (CN)

(21) Appl. No.: 19/148,045

(22) PCT Filed: Aug. 8, 2024
(86) PCT No.: PCT/CN2024/110793

§ 371 Date: Jul. 15, 2025

(30) Foreign Application Priority Data
Sep. 20, 2023 (CN) 202311220949.8

(51) Int. Cl. H10K 85/60; C07B 59/00; C07C 211/54; C07D 209/86; C07D 307/77; C07D 307/91; C07D 307/92; C07D 491/048; C07D 495/04; C07D 519/00; H10K 50/15; H10K 50/17

(52) U.S. Cl. CPC H10K 85/633; C07B 59/001; C07B 59/002; C07C 211/54; C07D 209/86; C07D 307/77; C07D 307/91; C07D 307/92; C07D 491/048; C07D 495/04; C07D 519/00; H10K 85/636; C07B 2200/05; C07C 2603/18; C07C 2603/20; C07C 2603/94; H10K 50/15; H10K 50/17; H10K 85/615; H10K 85/626; H10K 85/6572; H10K 85/6574

(57) ABSTRACT

Aromatic amine compounds, an electroluminescent device and a display apparatus, belonging to the technical field of display. The aromatic amine compounds can improve the performance of electroluminescent devices.

### CROSS-REFERENCE TO RELATED APPLICATIONS

The present disclosure is a U.S. National Stage Application of International Application No. PCT/CN2024/110793, filed on Aug. 8, 2024, which is based upon and claims priority to Chinese Patent Application No. 202311220949.8, entitled “Aromatic Amine Compound for Organic Electroluminescent Devices, Preparation Method and Application Thereof”, filed on Sep. 20, 2023. The entire contents of them are incorporated herein by reference.

### TECHNICAL FIELD

The present disclosure relates to the field of display technology, specifically to an aromatic amine compound, an electroluminescent device, and a display apparatus.

### BACKGROUND

Electroluminescent devices can self-emit light driven by electric current. The electroluminescent device generally includes at least an anode, a functional layer, and a cathode stacked sequentially. Holes provided by the anode and electrons provided by the cathode recombine in the functional layer, enabling the functional layer to emit light.

Compared with traditional liquid crystal displays, as a new generation of display technology, electroluminescent devices possess advantages including self-emission, wide viewing angles, low power consumption, high response rates, and full-color display, demonstrating immense research and development value as well as broad application prospects.

It should be noted that the information disclosed in the Background section above is only for enhancing the understanding of the background of the present disclosure, and thus may include information that does not constitute prior art known to those of ordinary skill in the art.

### SUMMARY

The present disclosure aims to provide an aromatic amine compound, an electroluminescent device, and a display apparatus to improve the performance of electroluminescent devices.

According to a first aspect of the present disclosure, an aromatic amine compound is provided. The aromatic amine compound has a structural formula represented by Chemical Formula A or Chemical Formula B:

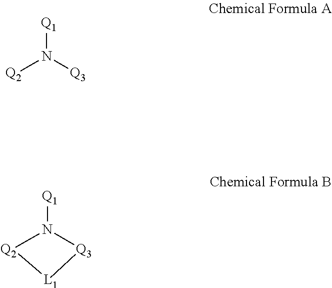

wherein L₁ is selected from a single bond, a substituted or unsubstituted alkyl group with 1~10 carbon atoms, a substituted or unsubstituted cycloalkyl group with 1~10 carbon atoms, a substituted or unsubstituted aryl group with 6~30 carbon atoms, or a substituted or unsubstituted heteroaryl group with 3~30 carbon atoms;
Q₁, Q₂, and Q₃ are each independently selected from a substituted or unsubstituted aryl group with 6~50 carbon atoms or a substituted or unsubstituted heteroaryl group with 3~50 carbon atoms, and at least one of Q₁, Q₂ or Q₃ contains at least one group represented by Chemical Formula P1 to Chemical Formula P6:

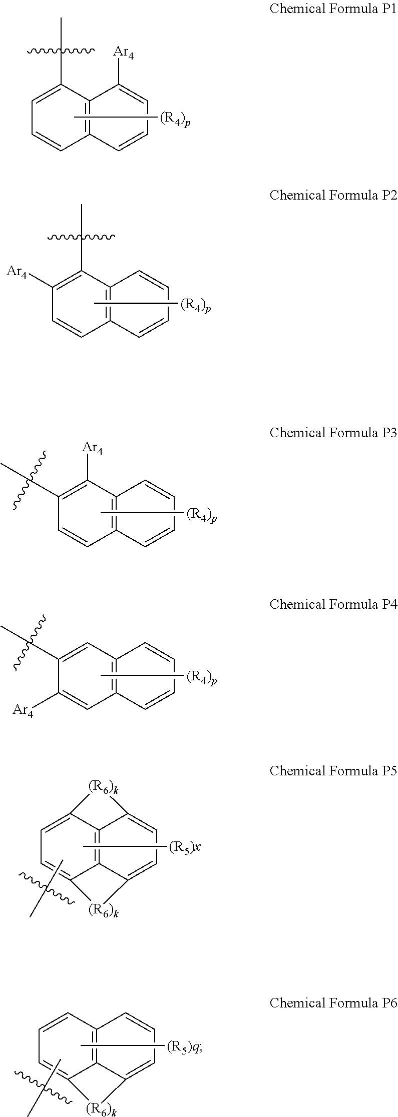

wherein Ar₄ is selected from hydrogen, deuterium, a substituted or unsubstituted alkyl group with 1~10 carbon atoms, a substituted or unsubstituted cycloalkyl group with 3~10 carbon atoms, a substituted or unsubstituted aryl group with 6~30 carbon atoms, a substituted or unsubstituted heteroaryl group with 3~30 carbon atoms, or a substituted or unsubstituted cycloalkane-naphthyl group with 14~30 carbon atoms;
R₄ and R₅ are the same or different, each independently selected from deuterium, a substituted or unsubstituted alkyl group with 1~20 carbon atoms, a substituted or unsubstituted cycloalkyl group with 3~10 carbon atoms, a substituted or unsubstituted aryl group with 6~20 carbon atoms, a substituted or unsubstituted heteraryl group with 3~20 carbon atoms, a substituted or unsubstituted silyl group, or a substituted or unsubstituted arylsilyl group with 6~20 carbon atoms;
R₆ is C(R₇R₈); R₇ and R₈ are each independently selected from hydrogen, deuterium, fluorine, an alkyl group with 1~4 carbon atoms, or a substituted or unsubstituted aryl group with 6~12 carbon atoms;
p represents an integer from 0 to 6; when p is greater than 1, any two R₄ groups may be the same or different;
q represents an integer from 0 to 4; when q is greater than 1, any two R₅ groups may be the same or different;
x represents an integer from 0 to 3; when x is greater than 1, any two R₅ groups may be the same or different;
k is an integer from 2 to 4; any two R₇ groups may be the same or different; any two R₈ groups may be the same or different;
when L₁, Q₁, Q₂, Q₃, R₄, R₅, R₇, and Ar₄ have substituents, these substituents may be the same or different and are each independently selected from deuterium, halogen, an alkylsilyl group with 1~30 carbon atoms, an arylsilyl group with 6~30 carbon atoms, an arylamino group with 6~30 carbon atoms, an alkyl group with 1~30 carbon atoms, a cycloalkyl group with 5~10 carbon atoms, an alkenyl group with 2~30 carbon atoms, an aryl group with 6~30 carbon atoms, or a heteroaryl group with 3~30 carbon atoms;

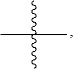

represents a connecting bond.

According to another aspect of the present disclosure electroluminescent device is provided. The device includes an organic layer containing the aforementioned aromatic amine compound.

According to a further aspect of the present disclosure, a display apparatus is provided, containing the aforementioned electroluminescent device.

It is to be understood that the general description above and the detailed description below are exemplary and, explanatory only, and do not limit the present disclosure.

### BRIEF DESCRIPTION OF THE DRAWINGS

The drawings here are incorporated into the specification and constitute a part of the specification, show embodiments in consistent with the present disclosure, and are used together with the specification to explain principles of the present disclosure. Obviously, the drawings in the following description are only some embodiments of the present disclosure. For those skilled in the art, other drawings can be obtained based on these drawings without creative work.

FIG. 1 is a schematic structural diagram of an electroluminescent device in an embodiment of the present disclosure.

FIG. 2 is a partial schematic structural diagram of a display panel in an embodiment of the present disclosure.

### DETAILED DESCRIPTION

Example embodiments will now be described more fully with reference to the accompanying drawings. However, the example embodiments can be embodied in a variety of forms and should not be construed as limited to the embodiments set forth herein; rather, these embodiments are provided so that the present disclosure will be thorough and complete and fully convey the concepts of the example embodiments to those skilled in the art. The described features, structures, or characteristics may be incorporated in one or more embodiments in any suitable manner. Like reference numerals refer to the same or similar structures, and detailed description thereof will be omitted. In addition, the drawings are merely schematic illustrations of the present disclosure and are not necessarily drawn to scale.

Although relative terms such as “upper” and “lower” are used in this specification to describe the relative relationship between one component and another component in the illustrated embodiments, these terms are used herein merely for convenience of description, for example, based on the orientation shown in the accompanying drawings. It should be understood that if the device in the diagram is turned upside down, the component described as being “upper” will become a “lower” component. When a structure is described as being “on” another structure, this may mean that the structure is integrally formed on the other structure, or that the structure is “directly” disposed on the other structure, or that the structure is “indirectly” disposed on the other structure through an intervening structure.

The terms “a”, “an”, “the”, “said”, and “at least one” are used to indicate that there are one or more elements/components/and the like; the terms “including” and “having” are used to refer to an open-ended inclusion and refer to that there may be additional elements/components/and the like in addition to the listed elements/components/and the like; the terms “first”, “second”, “third”, and the like are used only as labels, not to the number of objects thereof.

Example embodiments will now be described more fully with reference to the accompanying drawings. However, the example embodiments may be implemented in various forms and should not be construed as limited to the embodiments set forth herein. Instead, these embodiments are provided to ensure that the present disclosure is comprehensive and complete, and to fully convey the concepts of the example embodiments to those skilled in the art. The same reference numerals in the drawings denote the same or similar structures, and thus their detailed descriptions will be omitted. Furthermore, the drawings are schematic illustrations of the present disclosure and are not necessarily drawn to scale.

In the present disclosure, the descriptive phrase “ . . . each independently selected from” should be interpreted broadly. It may indicate that, among different groups, the specific options represented by identical symbols do not affect each other, or it may mean that, within the same group, the specific options represented by identical symbols do not affect each other.

For example, in the statement

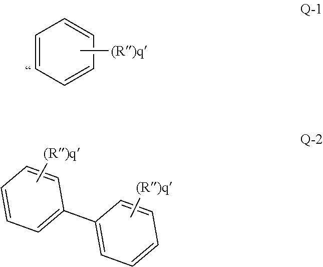

wherein each q′ is independently 0, 1, 2, or 3, and each R″ is independently selected from hydrogen, deuterium, fluorine, or chlorine”, the interpretation is as follows: Formula Q-1 indicates that there are q′ substituents R″ on the benzene ring, where each R″ may be the same or different, and the selection of each R″ is independent of the others; formula Q-2 indicates that each benzene ring in the biphenyl structure has q′ substituents R″, the number q′ of R″ substituents on the two benzene rings may be same or different, each R″ may be the same or different, and the selection of each R″ is independent of the others.

In the present disclosure, an unpositioned connecting bond refers to a single bond

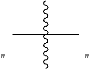

extending from a ring system, which means that one end of the connecting bond can be connected to any position of the ring system through which the bond passes, and the other end can be connected to the rest of the compound molecule.

For example, as shown in Formula (f) below, the naphthyl group represented by Formula (f) is connected to other positions of the molecule through two unpositioned connecting bonds which pass through a dicyclic ring. It means that it can include any possible connection way as shown in Formulas (f-1) to (f-10).

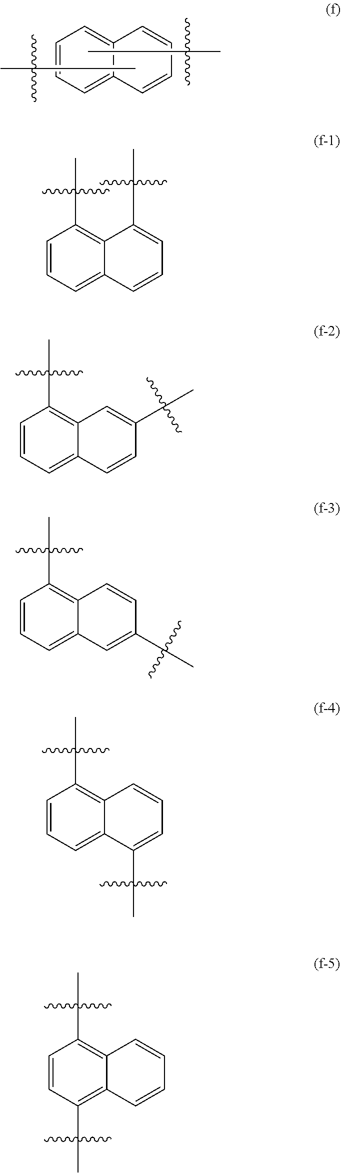

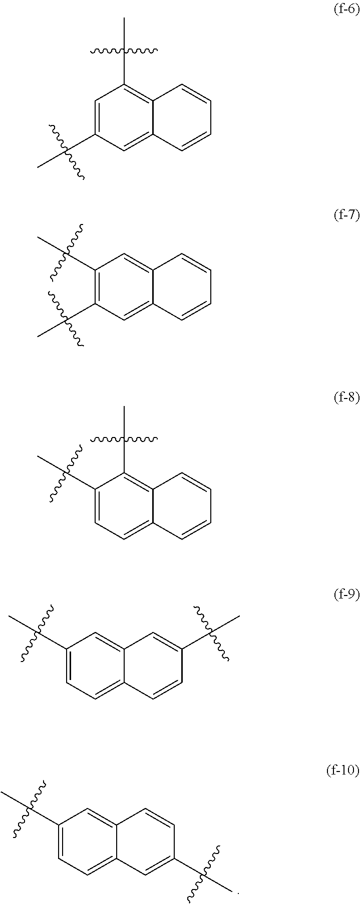

For example again, as shown in Formula (X′) below, the phenyl group represented by Formula (X′) is connected to other positions of the molecule through an unpositioned connecting bond extending from one side of the benzene ring. It means that it can include any possible connection way as shown in Formulas (X′-1) to (X′-4).

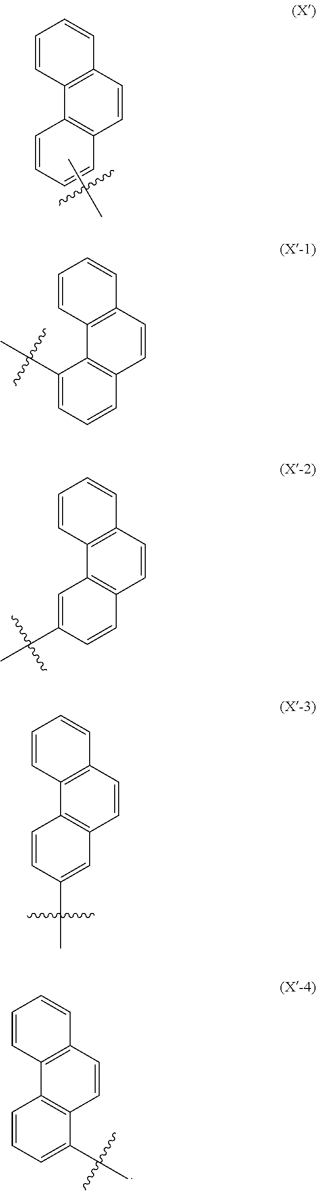

In the embodiment of the disclosure, the unpositioned substituent refers to a substituent connected by a single bond extending from the ring system, which means that the substituent can be connected at any possible position of the ring system. For example, as shown in Formula (Y) below, the substituent R′ group represented by Formula (Y) is connected to the quinoline ring through an unpositioned connecting bond. It means that it can include any possible connection way as shown in Formulas (Y-1) to (Y-7).

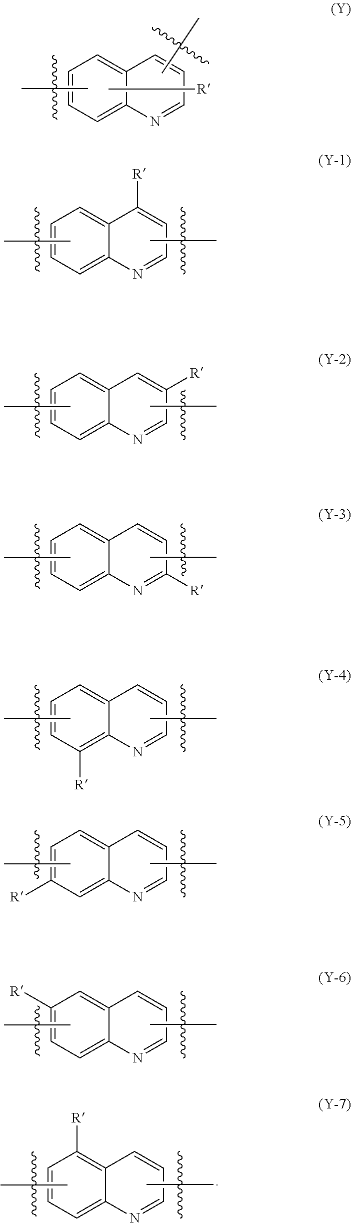

In the present disclosure, for a substituted or unsubstituted group G with M carbon atoms, the number M of carbon atoms of the G group does not count carbon atoms on substituents. For example, the total number of carbon atoms of a methyl-substituted group G having M carbon atoms is M+1. Correspondingly, for a substituted or unsubstituted aryl group or heteroaryl group having M carbon atoms, the number M of carbon atoms does not count carbon atoms on substituents, but counts only carbon atoms on the aromatic ring or the heteroaromatic ring. For instance:

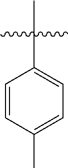 [SMILES: *c1ccc(C)cc1]

is an aryl group having 6 carbon atoms substituted with a methyl group;

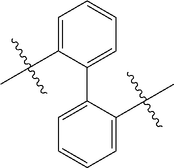 [SMILES: *c1ccccc1-c1ccccc1*]

is an unsubstituted arylene group having 12 carbon atoms.

In this disclosure, unless otherwise specifically defined, the term “hetero” refers to a functional group containing at least one heteroatom selected from B, N, O, S, Se, Si, or P, with the remaining atoms being carbon, hydrogen, and/or deuterium. An unsubstituted alkyl group may be a “saturated alkyl group” without any double or triple bonds.

In the present disclosure, “alkyl” may include a linear alkyl group or a branched alkyl group. The alkyl group may have 1 to 18 carbon atoms. In the present disclosure, numerical ranges such as “1-18” refer to each integer within the given range; for example, “an alkyl group having 1 to 18 carbon atoms” means alkyl groups that may contain 1 carbon atom, 2 carbon atoms, 3 carbon atoms, 4 carbon atoms, 5 carbon atoms, 6 carbon atoms, 7 carbon atoms, 8 carbon atoms, 9 carbon atoms, 10 carbon atoms, 11 carbon atoms, 12 carbon atoms, 13 carbon atoms, 14 carbon atoms, 15 carbon atoms, 16 carbon atoms, 17 carbon atoms, or 18 carbon atoms. In some examples, the alkyl group may also be a small alkyl group having 1 to 5 carbon atoms.

Optionally, the alkyl group is selected from alkyl groups having 1 to 5 carbon atoms. Specific examples include but are not limited to methyl, ethyl, n-propyl, isopropyl, n-butyl, isobutyl, sec-butyl, tert-butyl, and pentyl.

In the present disclosure, “cycloalkyl” refers to a group derived from a saturated cyclic carbon chain structure. The cycloalkyl group may have 5 to 10 carbon atoms. In the present disclosure, numerical ranges such as “5~10” in “a cycloalkyl group having 5 to 10 carbon atoms” refer to each integer within the given range; for example, “a cycloalkyl group having 5 to 10 carbon atoms” means cycloalkyl groups that may contain 5 carbon atoms, 6 carbon atoms, 7 carbon atoms, 8 carbon atoms, 9 carbon atoms, or 10 carbon atoms.

Optionally, specific examples of cycloalkyl groups include but are not limited to cyclopentyl, cyclohexyl, adamantyl, norbornyl, and the like.

In the present disclosure, “aryl” refers to an optional functional group or substituent derived from an aromatic carbon ring. The aryl group may be a monocyclic aryl group (e.g., phenyl) or a polycyclic aryl group. In other words, the aryl group may be a monocyclic aryl group, a fused-ring aryl group, two or more monocyclic aryl groups connected via conjugated carbon-carbon bonds, a monocyclic aryl group and a fused-ring aryl group connected via conjugated carbon-carbon bonds, or two or more fused-ring aryl groups connected via conjugated carbon-carbon bonds. That is, unless otherwise specified, two or more aromatic groups connected via conjugated carbon-carbon bonds may also be regarded as aryl groups in the present disclosure. Among these, fused-ring aryl groups may include bicyclic fused aryl groups (e.g., naphthyl), tricyclic fused aryl groups (e.g., phenanthrenyl, fluorenyl, anthracenyl), and the like. The aryl group does not contain heteroatoms such as B, N, O, S, P, Se, or Si. For example, in the present disclosure, biphenyl, terphenyl, and the like are considered aryl groups. Examples of aryl groups may include but are not limited to phenyl, naphthyl, fluorenyl, anthracenyl, phenanthrenyl, biphenyl, terphenyl, quaterphenyl, pentaphenyl, benzo[9,10]phenanthrenyl, pyrenyl, benzofluoranthenyl, chrysyl, and the like. In the present disclosure, “biphenyl” may be understood as a phenyl-substituted aryl group or as an unsubstituted aryl group. In the present disclosure, the term “arylene” refers to a divalent group formed by an aryl group losing one additional hydrogen atom.

In the present disclosure, a substituted aryl group may be an aryl group in which one or two or more hydrogen atoms are substituted with groups such as deuterium atoms, aryl groups, heteroaryl groups, alkyl groups, cycloalkyl groups, alkoxy groups, or other substituents.

In the present disclosure, “heteroaryl” refers to a monovalent aromatic ring or its derivative containing at least one heteroatom in the ring, where the heteroatom may be at least one selected from B, O, N, P, Si, Se, and S. The heteroaryl group may be a monocyclic heteroaryl group or a polycyclic heteroaryl group. In other words, the heteroaryl group may be a single aromatic ring system or multiple aromatic ring systems connected via conjugated carbon-carbon bonds, where each aromatic ring system is an aromatic monocycle or an aromatic fused ring. Examples of heteroaryl groups may include thienyl, furyl, pyrrolyl, imidazolyl, thiazolyl, oxazolyl, oxadiazolyl, triazolyl, pyridyl, bipyridyl, pyrimidinyl, triazinyl, acridinyl, pyridazinyl, pyrazinyl, quinolinyl, quinazolinyl, quinoxalinyl, phenoxazinyl, phthalazinyl, pyridopyrimidinyl, pyridopyrazinyl, pyrazinopyrazinyl, isoquinolinyl, indolyl, carbazolyl, benzoxazolyl, benzimidazolyl, benzothiazolyl, benzocarbazolyl, benzothienyl, dibenzothienyl, thienothienyl, benzofuryl, phenanthrolinyl, isoxazolyl, thiadiazolyl, benzothiazolyl, phenothiazinyl, silafluorenyl, dibenzofuryl, N-aryl carbazolyl (e.g., N-phenyl carbazolyl), N-heteroaryl carbazolyl (e.g., N-pyridyl carbazolyl), N-alkyl carbazolyl (e.g., N-methyl carbazolyl), and the like, without limitation. Among these, thienyl, furyl, phenanthrolinyl, and the like are heteroaryl groups of the single aromatic ring system type, while N-aryl carbazolyl (e.g., N-phenyl carbazolyl) and N-heteroaryl carbazolyl are heteroaryl groups of the polycyclic system type connected via conjugated carbon-carbon bonds.

In the present disclosure, a substituted heteroaryl group may be a heteroaryl group in which one or two or more hydrogen atoms are substituted with groups such as deuterium atoms, aryl groups, heteroaryl groups, alkyl groups, cycloalkyl groups, alkoxy groups, and the like.

The present disclosure provides an aromatic amine compound that can be applied to an electroluminescent device to improve its performance. Specifically, at least a portion of the film layers in the electroluminescent device may employ the aromatic amine compound provided in the present disclosure. For example, the electroluminescent device includes an organic layer containing the aromatic amine compound provided in the present disclosure. The organic layer containing the aromatic amine compound may further include other components or may solely consist of the aromatic amine compound provided in the embodiments of the present disclosure.

Optionally, the electroluminescent device may at least include two electrodes and a functional layer sandwiched between the two electrodes. The organic layer in the electroluminescent device employing the aromatic amine compound provided in the present disclosure may be located within the functional layer between the two electrodes or outside the two electrodes to improve light extraction efficiency.

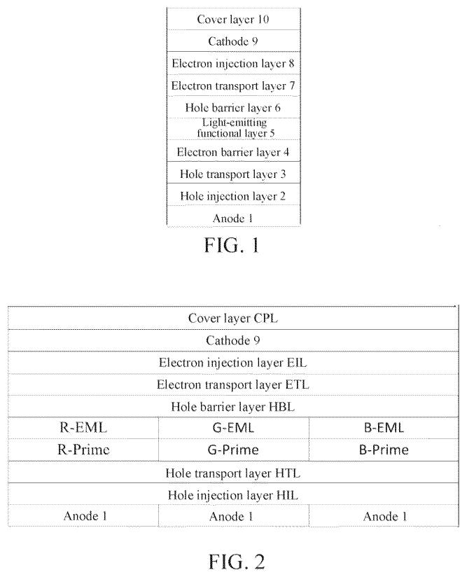

Optionally, as shown in FIG. 1, the two electrodes (first electrode and second electrode) of the electroluminescent device may correspond to the anode 1 and cathode 9 of the device, respectively. For example, the first electrode may serve as the anode, and the second electrode may serve as the cathode. The anode is configured to supply holes to the functional layer, while the cathode is configured to supply electrons to the functional layer. Electrons and holes recombine in the functional layer to emit light.

In the present disclosure, as shown in FIG. 1, the functional layer of the electroluminescent device may include a light-emitting functional layer 5, where electrons and holes can recombine to form excitons, thereby causing the light-emitting functional layer to emit light. Optionally, the light-emitting functional layer may be a small-molecule organic light-emitting functional layer, a macromolecular polymer light-emitting functional layer, or a quantum dot light-emitting functional layer. Thus, the electroluminescent device may be an organic light-emitting diode (OLED), a polymer light-emitting diode (PLED), or a quantum dot light-emitting diode (QLED).

In some embodiments of the present disclosure, as shown in FIG. 1, the functional layer of the electroluminescent device may further include a first functional layer (hole transport region) located between the light-emitting functional layer and the anode. The first functional layer can inject holes from the anode into the light-emitting functional layer. For example, the first functional layer may include at least one of a hole injection layer 2, a hole transport layer 3, or an electron barrier layer 4. When the first functional layer includes all three of the hole injection layer, hole transport layer, and electron barrier layer, the anode, hole injection layer, hole transport layer, electron barrier layer, and light-emitting functional layer are sequentially stacked. Some aromatic amine compounds provided in the present disclosure are particularly suitable for application in the first functional layer and can achieve at least one of the following effects: improving the light-emitting efficiency of the electroluminescent device, enhancing the device lifetime, or reducing the driving voltage.

In some embodiments of the present disclosure, as shown in FIG. 1, the functional layer of the electroluminescent device may further include a second functional layer (electron transport region) located between the light-emitting functional layer and the cathode. The second functional layer can inject electrons from the cathode into the light-emitting functional layer. For example, the second functional layer may include at least one of an electron injection layer 8, an electron transport layer 7, or a hole barrier layer 6. When the second functional layer includes all three of the electron injection layer, electron transport layer, and hole barrier layer, the cathode, electron injection layer, electron transport layer, hole barrier layer, and light-emitting functional layer are sequentially stacked. In the embodiments of the present disclosure, the materials for each film layer in the second functional layer may be selected as needed. Any layer may be composed of organic materials, inorganic materials, or a mixture of organic and inorganic materials.

In some embodiments of the present disclosure, the electroluminescent device may also have a multi-stacked structure, where multiple functional layers are stacked between the two electrodes, and a charge generation layer may be disposed between every two adjacent functional layers. In this embodiment, the charge generation layer can improve electron-hole separation efficiency, thereby functioning similarly to an anode or cathode. The charge generation layer, adjacent to the first functional layer, can inject holes into the first functional layer, thus acting similarly to an anode relative to the first functional layer. The charge generation layer, adjacent to the second functional layer, can inject electrons into the second functional layer, thus acting similarly to a cathode relative to the second functional layer. When the electroluminescent device adopts a multi-stacked structure, the structures of any two functional layers may be the same or different.

In some embodiments of the present disclosure, as shown in FIG. 1, the electroluminescent device further includes a cover layer 10 disposed on the light-emitting side. Some aromatic amine compounds provided in the present disclosure are particularly suitable for application in this cover layer to improve the light extraction efficiency or enhance the lifetime of the electroluminescent device. In an example, the electroluminescent device includes sequentially stacked layers: a first electrode, a light-emitting functional layer, a second electrode, and a cover layer. The cover layer is located on the light-emitting side of the electroluminescent device and includes one or more aromatic amine compounds provided in the present disclosure. In this example, the second electrode may be a cathode or an anode. In other words, when the device emits light from the first electrode side, the cover layer is disposed on the side of the first electrode opposite to the second electrode. Conversely, when the device emits light from the second electrode side, the cover layer is disposed on the side of the second electrode opposite to the first electrode.

In the example shown in FIG. 1, the electroluminescent device emits light from the cathode side. It is understood that in other examples of the present disclosure, the electroluminescent device may also emit light from the anode side.

The embodiments of the present disclosure provide an aromatic amine compound, wherein the aromatic amine compound has a structural formula as shown in Chemical Formula A or Chemical Formula B:

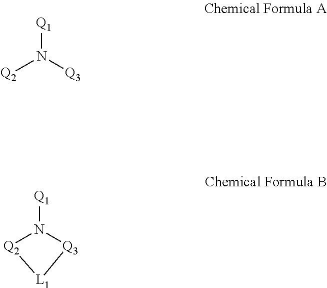

wherein L₁ is selected from a single bond, a substituted or unsubstituted alkyl group having 1 to 10 carbon atoms, a substituted or unsubstituted cycloalkyl group having 1 to 10 carbon atoms, a substituted or unsubstituted aryl group having 6 to 30 carbon atoms, or a substituted or unsubstituted heteroaryl group having 3 to 30 carbon atoms;
Q₁, Q₂, and Q₃ are each independently selected from a substituted or unsubstituted aryl group having 6 to 50 carbon atoms or a substituted or unsubstituted heteroaryl group having 3 to 50 carbon atoms, and at least one of Q₁, Q₂, and Q₃ contains at least one group represented by Chemical Formulas P1 to P6:

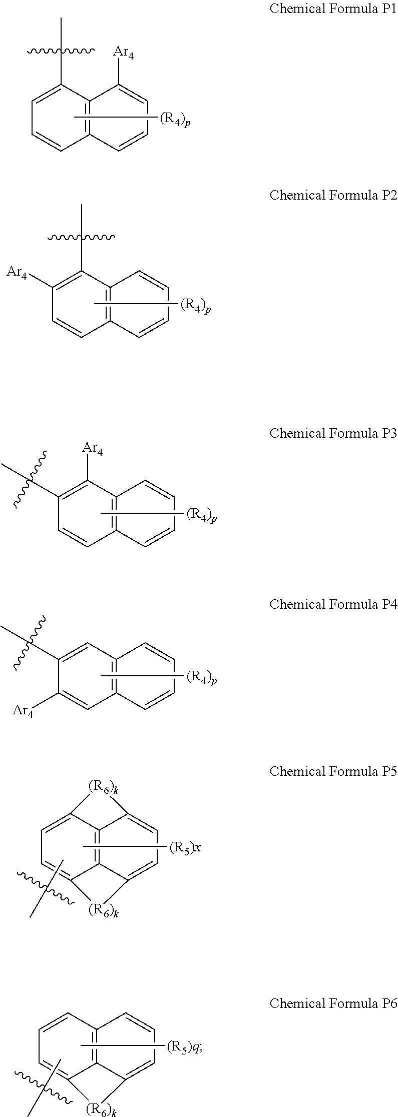

wherein Ar₄ is selected from hydrogen, deuterium, a substituted or unsubstituted alkyl group having 1 to 10 carbon atoms, a substituted or unsubstituted cycloalkyl group having 3 to 10 carbon atoms, a substituted or unsubstituted aryl group having 6 to 30 carbon atoms, a substituted or unsubstituted heteroaryl group having 3 to 30 carbon atoms, or a substituted or unsubstituted cycloalkane-fused naphthyl group having 14 to 30 carbon atoms;
R₄ and R₅ are the same or different and are each independently selected from deuterium, a substituted or unsubstituted alkyl group having 1 to 20 carbon atoms, a substituted or unsubstituted cycloalkyl group having 3 to 10 carbon atoms, a substituted or unsubstituted aryl group having 6 to 20 carbon atoms, a substituted or unsubstituted heteroaryl group having 3 to 20 carbon atoms, a substituted or unsubstituted silyl group, or a substituted or unsubstituted arylsilyl group having 6 to 20 carbon atoms;
R₆ is C(R₇R₈); R₇ and R₈ are each independently selected from hydrogen, deuterium, fluorine, an alkyl group having 1 to 4 carbon atoms, or a substituted or unsubstituted aryl group having 6 to 12 carbon atoms;
p represents an integer from 0 to 6; when p is greater than 1, any two R₄ groups are the same or different;
q represents an integer from 0 to 4; when q is greater than 1, any two R₅ groups are the same or different;
x represents an integer from 0 to 3; when x is greater than 1, any two R₅ groups are the same or different;
k is an integer from 2 to 4; any two R₇ groups are the same or different; any two R₈ groups are the same or different;
when L₁, Q₁, Q₂, Q₃, R₄, R₅, R₇, R₈, or Ar₄ has substituents, the substituents may be the same or different and are each independently selected from deuterium, halogen, an alkylsilyl group having 1 to 30 carbon atoms, an arylsilyl group having 6 to 30 carbon atoms, an arylamino group having 6 to 30 carbon atoms, an alkyl group having 1 to 30 carbon atoms, a cycloalkyl group having 5 to 10 carbon atoms, an alkenyl group having 2 to 30 carbon atoms, an aryl group having 6 to 30 carbon atoms, or a heteroaryl group having 3 to 30 carbon atoms;

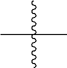

represents a bonding site.

The aromatic amine compound provided in the embodiments of the present disclosure has a structure represented by Chemical Formula A or Chemical Formula B and includes at least one group represented by Chemical Formulas P1 to P6. Introducing at least one of the groups shown in Chemical Formulas P1 to P6 into the triarylamine structure can effectively enhance the film-forming properties of the compound. Specifically, in the groups represented by Chemical Formulas P1 to P4, the Ar₄ group on the naphthalene ring creates large steric hindrance with the triarylamine backbone. This results in distinct dihedral angles between the planes of the triarylamine backbone, the naphthalene ring, and the Ar₄ group, thereby increasing the three-dimensionality of the aromatic amine compound, reducing intermolecular stacking, and improving film-forming performance. In the groups represented by Chemical Formulas P5 to P6, the fused cycloalkyl structure on the benzene ring similarly enhances the three-dimensionality of the aromatic amine compound, reducing intermolecular stacking and improving film-forming properties. Additionally, in certain specific compounds, the aromatic amine compound exhibits improved electrochemical or optical characteristics, enabling enhancement of at least one performance parameter when applied to organic layers in electroluminescent devices.

In an embodiment of the present disclosure, Ar₄ is selected from a substituted or unsubstituted aryl group having 6 to 30 carbon atoms, a substituted or unsubstituted heteroaryl group having 3 to 30 carbon atoms, or a substituted or unsubstituted cycloalkane-fused naphthyl group having 14 to 30 carbon atoms.

In another embodiment, R₆ is CH₂ or CD₂, and k is 2. In this configuration, the fused cycloalkane-naphthalene group (e.g., acenaphthenylene, deuterated acenaphthenylene) introduced into the aromatic amine compound exhibits strong electron-donating capabilities (increased electron density), which enhances hole mobility. Compared to naphthalene, acenaphthenylene or deuterated acenaphthenylene increases molecular weight, improving the thermal stability (higher glass transition temperature) of the aromatic amine compound and ensuring better device lifetime. The fused cycloalkane-naphthalene structure also increases molecular weight (while improving material solubility for easier cleaning of evaporation masks) without significantly affecting sublimation temperature, facilitating evaporation processes. The groups represented by Chemical Formulas P5 to P6 enhance material anisotropy through twisted structures and steric hindrance, effectively increasing lateral resistance. When applied to the first functional layer (e.g., hole injection or transport layers), these compounds mitigate device crosstalk and lateral leakage. Furthermore, modifications with deuterium (D) or stable fragments enable the synthesis of high-performance organic materials suitable as hole-transport materials. Additionally, the groups in Chemical Formulas P5 to P6 impart a high refractive index to the aromatic amine compound, making it suitable for use in cover layers.

In an embodiment, a first type of aromatic amine compounds is provided, with a structure represented by Chemical Formula I:

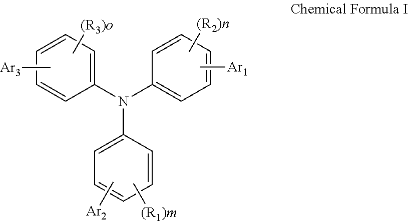

wherein Ar₃ is selected from any one of the following groups:

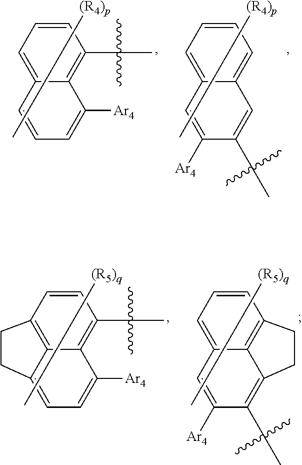

Ar₁ and Ar₂ may be the same or different and each independently represent hydrogen, deuterium, a substituted or unsubstituted alkyl group having 1 to 10 carbon atoms, a substituted or unsubstituted cycloalkyl group having 1 to 10 carbon atoms, a substituted or unsubstituted aryl group having 6 to 30 carbon atoms, a substituted or unsubstituted heteroaryl group having 3 to 30 carbon atoms, or a substituted or unsubstituted cycloalkylnaphthalene structure. The heteroaryl group contains at least one heteroatom selected from O, S, N, or Si;
R₁-R₃ may be the same or different and each independently represent deuterium, a substituted or unsubstituted alkyl group having 1 to 20 carbon atoms, a substituted or unsubstituted cycloalkyl group having 3 to 10 carbon atoms, a substituted or unsubstituted silyl group, or a substituted or unsubstituted arylsilyl group having 6 to 20 carbon atoms; when substituents are present on Ar₁, Ar₂, Ar₄, or R₁-R₅, the substituents may be the same or different and are each independently selected from deuterium, halogen, an alkylsilyl group having 1 to 30 carbon atoms, an arylsilyl group having 6 to 30 carbon atoms, an arylamino group having 6 to 30 carbon atoms, an alkyl group having 1 to 30 carbon atoms, an alkenyl group having 2 to 30 carbon atoms, or an aryl group having 6 to 30 carbon atoms;
when Ar₃ is selected as

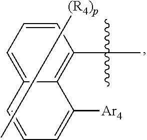

at least one benzene ring directly bonded to the naphthyl group has all hydrogens substituted with deuterium;
m represents an integer from 0 to 5;
n represents an integer from 0 to 5;
o represents an integer from 0 to 4.

The first type of aromatic amine compound introduces a naphthyl group or cycloalkylnaphthalene structure (e.g., alkyl naphthalene structures) with large steric hindrance. When the connecting positions include at least 1,8-positions or 2,3-positions, the resulting bond angles effectively prevent intermolecular stacking, enhancing film-forming properties. Additionally, these compounds exhibit excellent hole transport capability, appropriate HOMO energy levels and T1 values, and high glass transition temperatures (Tg). These properties can significantly improve the luminous efficiency of OLED devices, reduce driving voltage, and extend operational lifetime.

Technicians have observed that when Ar₃ is selected as a naphthyl or cycloalkylnaphthalene structure with large steric hindrance and connected via at least 1,8-positions or 2,3-positions, the resulting bond angles minimize intermolecular stacking and improve film-forming performance. Furthermore, replacing all hydrogens on the benzene ring directly bonded to the naphthyl or cycloalkylnaphthalene structure with deuterium enhances device lifetime. This effect is more pronounced when the benzene ring is connected via 1,8- or 2,3-positions and fully deuterated. Notably, introducing a naphthyl or cycloalkylnaphthalene structure at the 2,3-positions in Ar₃ outperforms analogous groups at the 1,8-positions in improving device performance. This is attributed to the greater adjustability of physical properties in 2,3-positioned structures, which effectively suppress exciton recombination at interfaces and further enhance charge mobility. Thus, the aromatic amine compound disclosed herein, when used as a hole-transport material, improves luminous efficiency, reduces energy consumption, lowers driving voltage, and extends device lifetime. Additionally, introducing cycloalkylnaphthalene structures at the Ar₃ position not only retains the advantages of naphthyl-based structures but also improves the compound's solubility (facilitating evaporation mask cleaning) and optimizes sublimation temperature, thereby enhancing device stability.

Optionally, this first type of aromatic amine compound is particularly suitable for application in the first functional layer, especially in the hole transport layer or electron barrier layer.

In an embodiment of the present disclosure, in the first type of aromatic amine compound, the aryl group is selected from any one of methylfluorenyl, a cycloalkylfluorenyl group having 5 to 10 carbon atoms, 9,9-diphenylfluorenyl, or spirobifluorenyl.

In an embodiment of the present disclosure, in the first type of aromatic amine compound, the cycloalkylnaphthalene is selected from one of the following structures:

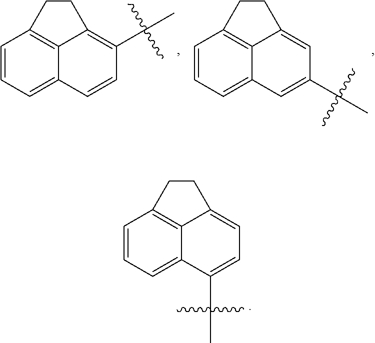 [SMILES: *c1ccc2cccc3c2c1CC3 ; *c1cc2c3c(cccc3c1)CC2 ; *c1ccc2c3c(cccc13)CC2]

In an embodiment of the present disclosure, in the first type of aromatic amine compound, Ar₁, Ar₂, and Ar₄ are each independently selected from a substituted or unsubstituted structure represented by one of formulas A-1 to A-34:

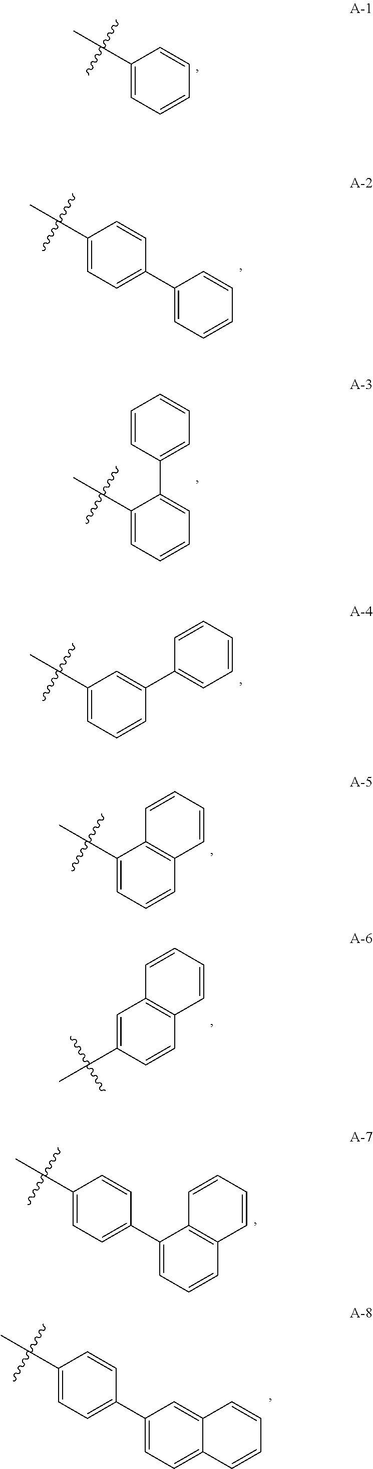

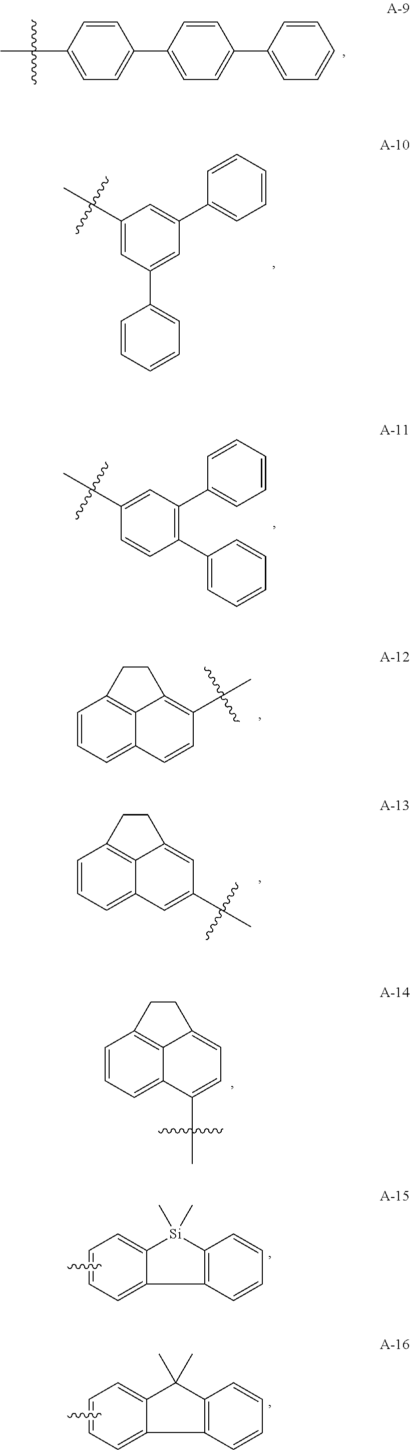

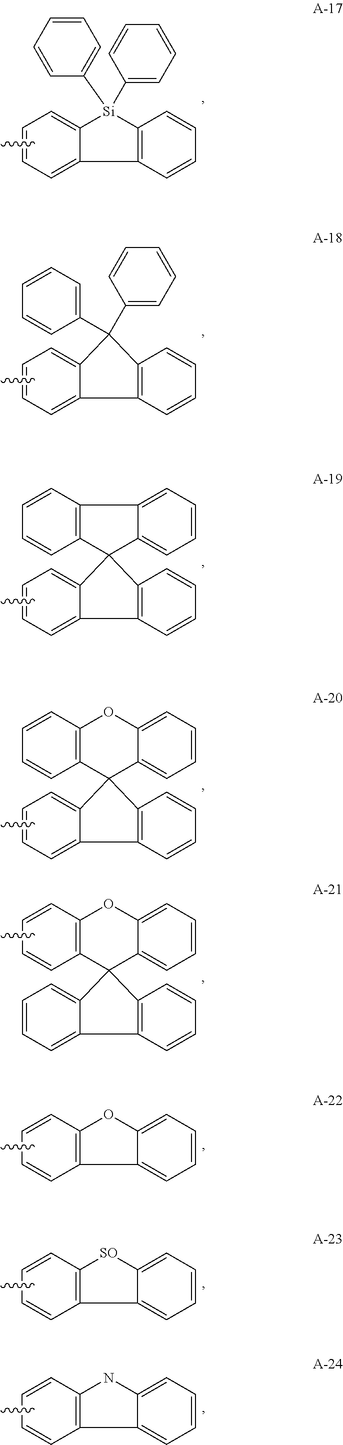

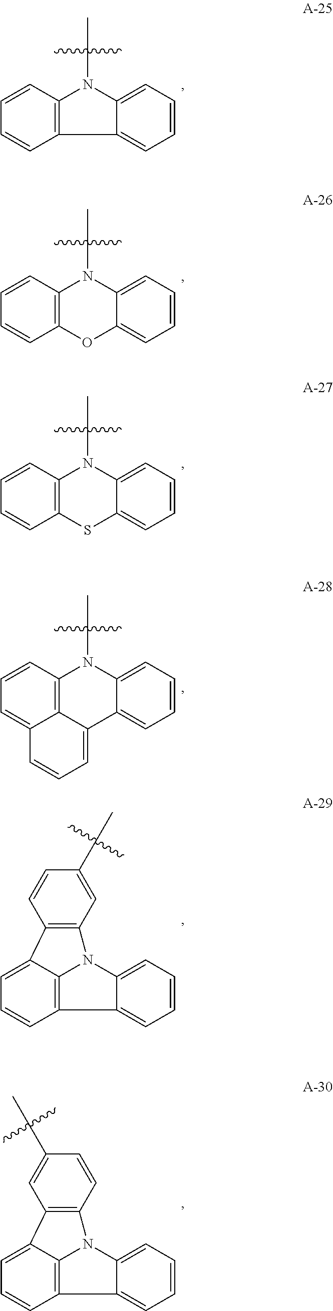 [SMILES: *n1c2ccccc2c2ccccc21 ; *N1c2ccccc2Oc2ccccc21 ; *N1c2ccccc2Sc2ccccc21 ; *N1c2ccccc2-c2cccc3cccc1c23 ; *c1ccc2c3cccc4c5ccccc5n(c2c1)c43 ; *c1ccc2c(c1)c1cccc3c4ccccc4n2c31]

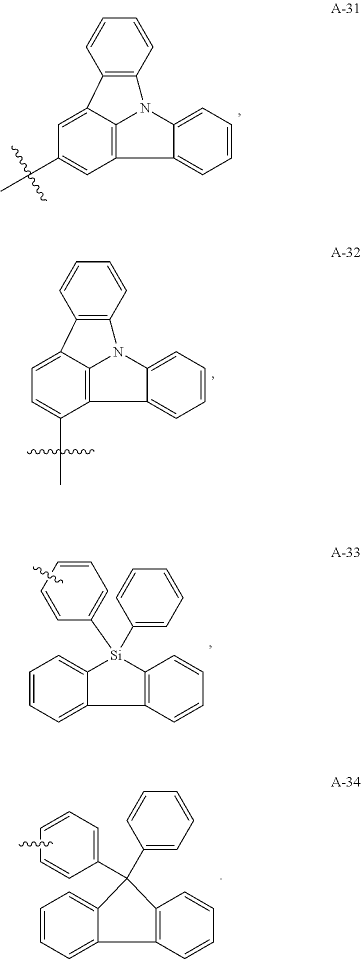

In an example, when Formulas A-1 to A-34 have substituents, the substituents on Formulas A-1 to A-34 may be deuterium. For instance, Ar₁, Ar₂, and Ar₄ are each independently selected from a substituted or unsubstituted group A′. An unsubstituted group A′ is independently selected from Formulas A-1 to A-34, while a substituted group A′ is formed by replacing at least one hydrogen on the unsubstituted group A′ with deuterium.

In an embodiment of the present disclosure, the first type of aromatic amine compound is selected from one of the following structures:

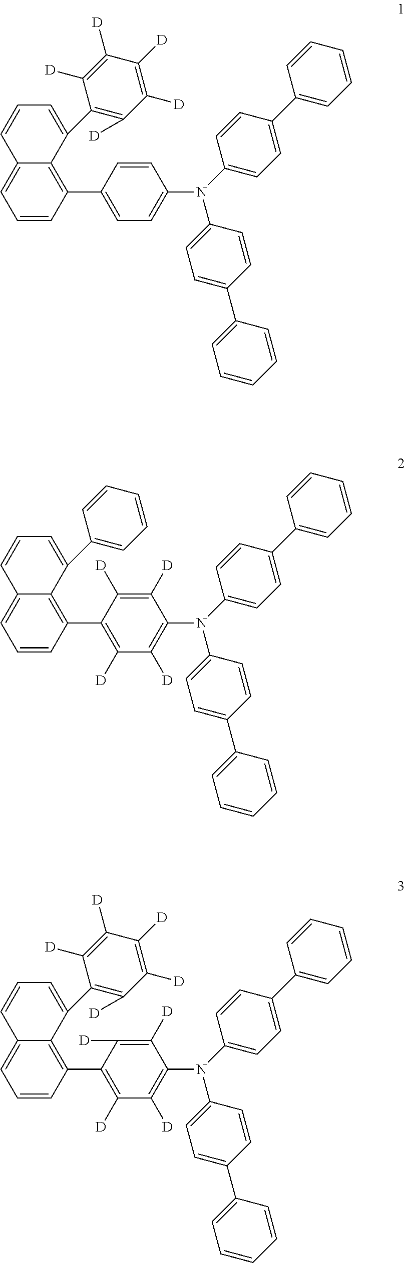 [SMILES: [2H]c1c([2H])c([2H])c(-c2cccc3cccc(-c4ccc(N(c5ccc(-c6ccccc6)cc5)c5ccc(-c6ccccc6)cc5)cc4)c23)c([2H])c1[2H] ; [2H]c1c([2H])c(N(c2ccc(-c3ccccc3)cc2)c2ccc(-c3ccccc3)cc2)c([2H])c([2H])c1-c1cccc2cccc(-c3ccccc3)c12 ; [2H]c1c([2H])c([2H])c(-c2cccc3cccc(-c4c([2H])c([2H])c(N(c5ccc(-c6ccccc6)cc5)c5ccc(-c6ccccc6)cc5)c([2H])c4[2H])c23)c([2H])c1[2H]]

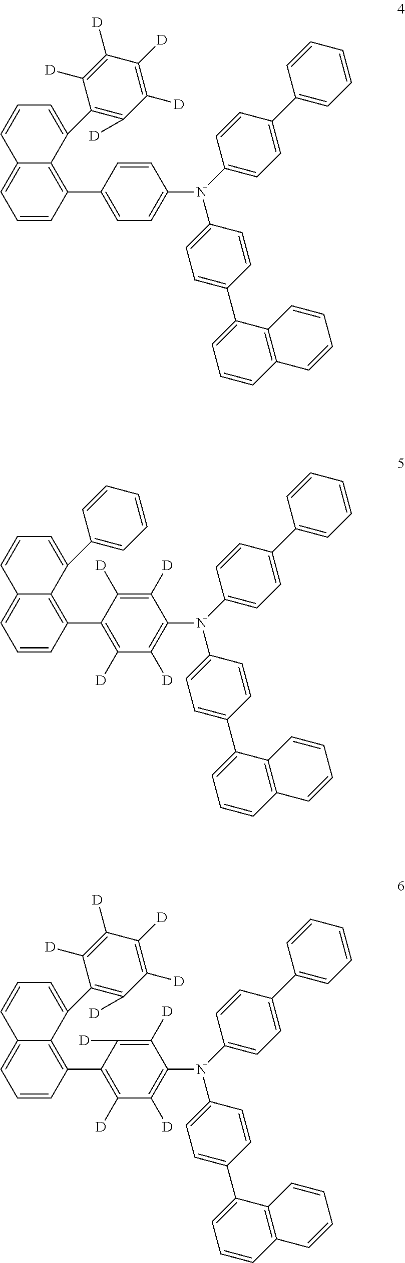 [SMILES: [2H]c1c([2H])c([2H])c(-c2cccc3cccc(-c4ccc(N(c5ccc(-c6ccccc6)cc5)c5ccc(-c6cccc7ccccc67)cc5)cc4)c23)c([2H])c1[2H] ; [2H]c1c([2H])c(N(c2ccc(-c3ccccc3)cc2)c2ccc(-c3cccc4ccccc34)cc2)c([2H])c([2H])c1-c1cccc2cccc(-c3ccccc3)c12 ; [2H]c1c([2H])c([2H])c(-c2cccc3cccc(-c4c([2H])c([2H])c(N(c5ccc(-c6ccccc6)cc5)c5ccc(-c6cccc7ccccc67)cc5)c([2H])c4[2H])c23)c([2H])c1[2H]]

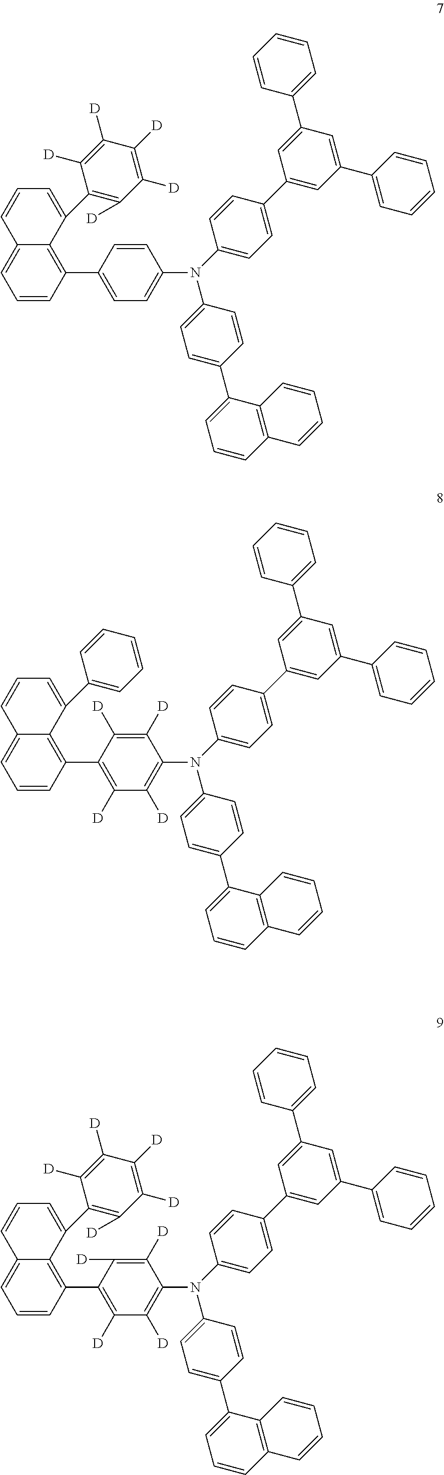 [SMILES: [2H]c1c([2H])c([2H])c(-c2cccc3cccc(-c4ccc(N(c5ccc(-c6cc(-c7ccccc7)cc(-c7ccccc7)c6)cc5)c5ccc(-c6cccc7ccccc67)cc5)cc4)c23)c([2H])c1[2H] ; [2H]c1c([2H])c(N(c2ccc(-c3cc(-c4ccccc4)cc(-c4ccccc4)c3)cc2)c2ccc(-c3cccc4ccccc34)cc2)c([2H])c([2H])c1-c1cccc2cccc(-c3ccccc3)c12 ; [2H]c1c([2H])c([2H])c(-c2cccc3cccc(-c4c([2H])c([2H])c(N(c5ccc(-c6cc(-c7ccccc7)cc(-c7ccccc7)c6)cc5)c5ccc(-c6cccc7ccccc67)cc5)c([2H])c4[2H])c23)c([2H])c1[2H]]

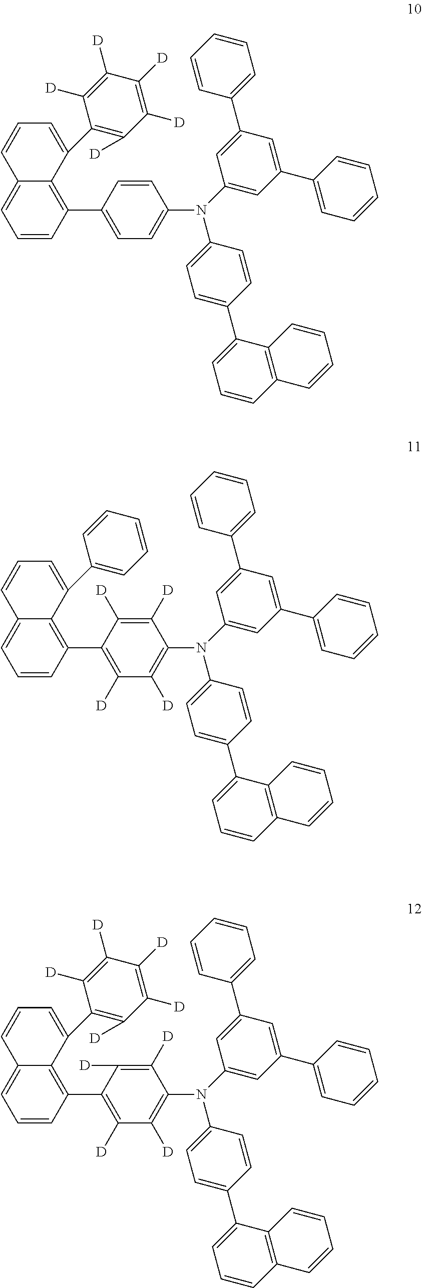 [SMILES: [2H]c1c([2H])c([2H])c(-c2cccc3cccc(-c4ccc(N(c5ccc(-c6cccc7ccccc67)cc5)c5cc(-c6ccccc6)cc(-c6ccccc6)c5)cc4)c23)c([2H])c1[2H] ; [2H]c1c([2H])c(N(c2ccc(-c3cccc4ccccc34)cc2)c2cc(-c3ccccc3)cc(-c3ccccc3)c2)c([2H])c([2H])c1-c1cccc2cccc(-c3ccccc3)c12 ; [2H]c1c([2H])c([2H])c(-c2cccc3cccc(-c4c([2H])c([2H])c(N(c5ccc(-c6cccc7ccccc67)cc5)c5cc(-c6ccccc6)cc(-c6ccccc6)c5)c([2H])c4[2H])c23)c([2H])c1[2H]]

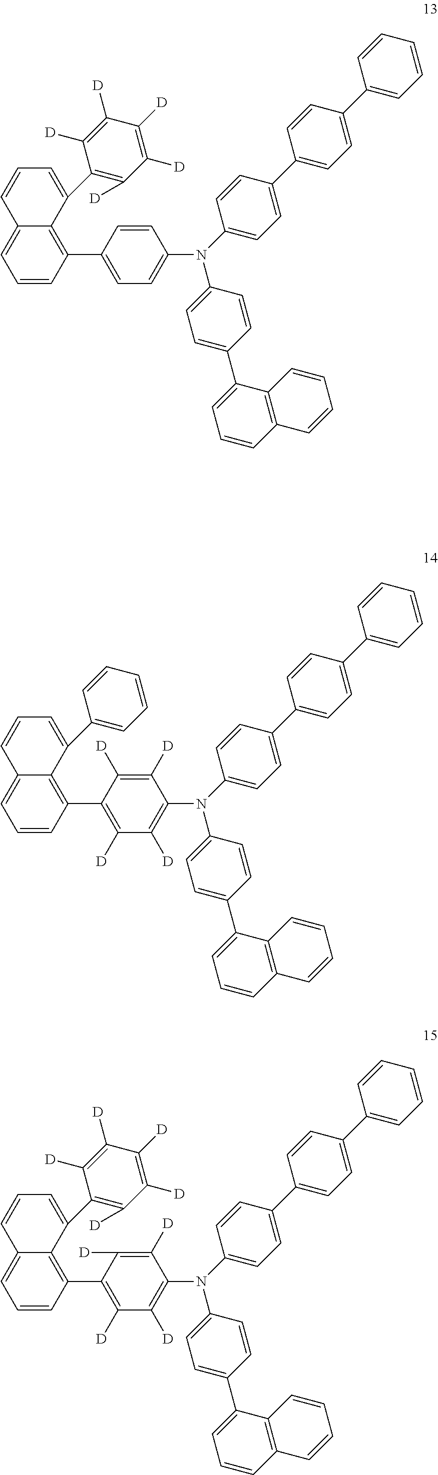 [SMILES: [2H]c1c([2H])c([2H])c(-c2cccc3cccc(-c4ccc(N(c5ccc(-c6ccc(-c7ccccc7)cc6)cc5)c5ccc(-c6cccc7ccccc67)cc5)cc4)c23)c([2H])c1[2H] ; [2H]c1c([2H])c(N(c2ccc(-c3ccc(-c4ccccc4)cc3)cc2)c2ccc(-c3cccc4ccccc34)cc2)c([2H])c([2H])c1-c1cccc2cccc(-c3ccccc3)c12 ; [2H]c1c([2H])c([2H])c(-c2cccc3cccc(-c4c([2H])c([2H])c(N(c5ccc(-c6ccc(-c7ccccc7)cc6)cc5)c5ccc(-c6cccc7ccccc67)cc5)c([2H])c4[2H])c23)c([2H])c1[2H]]

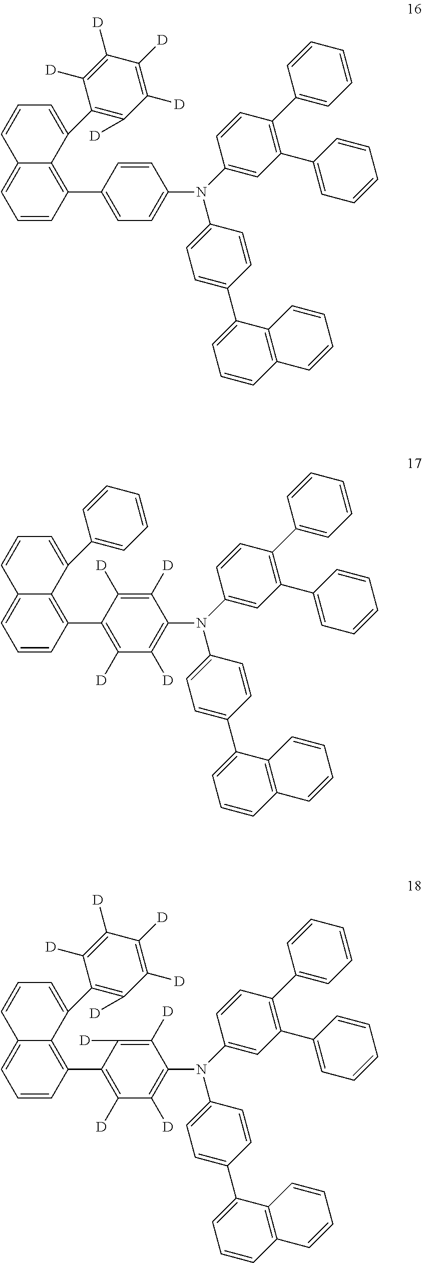 [SMILES: [2H]c1c([2H])c([2H])c(-c2cccc3cccc(-c4ccc(N(c5ccc(-c6cccc7ccccc67)cc5)c5ccc(-c6ccccc6)c(-c6ccccc6)c5)cc4)c23)c([2H])c1[2H] ; [2H]c1c([2H])c(N(c2ccc(-c3cccc4ccccc34)cc2)c2ccc(-c3ccccc3)c(-c3ccccc3)c2)c([2H])c([2H])c1-c1cccc2cccc(-c3ccccc3)c12 ; [2H]c1c([2H])c([2H])c(-c2cccc3cccc(-c4c([2H])c([2H])c(N(c5ccc(-c6cccc7ccccc67)cc5)c5ccc(-c6ccccc6)c(-c6ccccc6)c5)c([2H])c4[2H])c23)c([2H])c1[2H]]

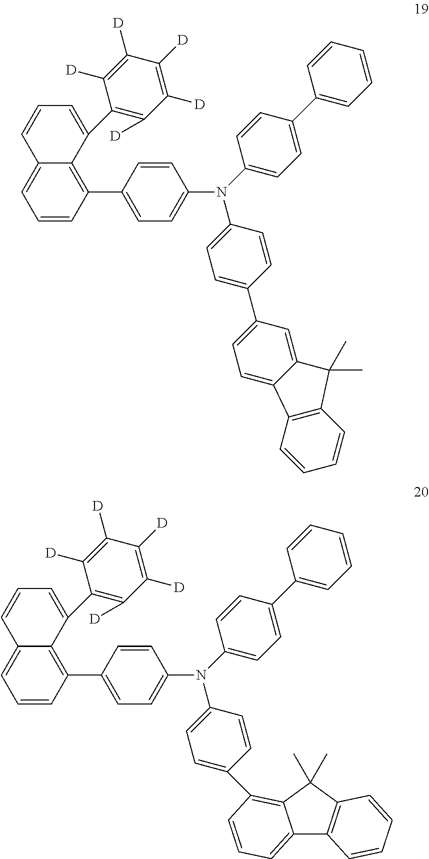 [SMILES: [2H]c1c([2H])c([2H])c(-c2cccc3cccc(-c4ccc(N(c5ccc(-c6ccccc6)cc5)c5ccc(-c6ccc7c(c6)C(C)(C)c6ccccc6-7)cc5)cc4)c23)c([2H])c1[2H] ; [2H]c1c([2H])c([2H])c(-c2cccc3cccc(-c4ccc(N(c5ccc(-c6ccccc6)cc5)c5ccc(-c6cccc7c6C(C)(C)c6ccccc6-7)cc5)cc4)c23)c([2H])c1[2H]]

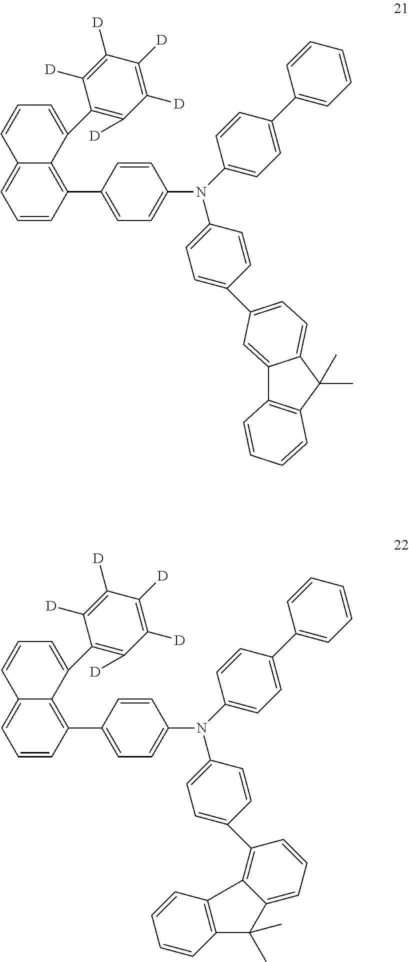 [SMILES: [2H]c1c([2H])c([2H])c(-c2cccc3cccc(-c4ccc(N(c5ccc(-c6ccccc6)cc5)c5ccc(-c6ccc7c(c6)-c6ccccc6C7(C)C)cc5)cc4)c23)c([2H])c1[2H] ; [2H]c1c([2H])c([2H])c(-c2cccc3cccc(-c4ccc(N(c5ccc(-c6ccccc6)cc5)c5ccc(-c6cccc7c6-c6ccccc6C7(C)C)cc5)cc4)c23)c([2H])c1[2H]]

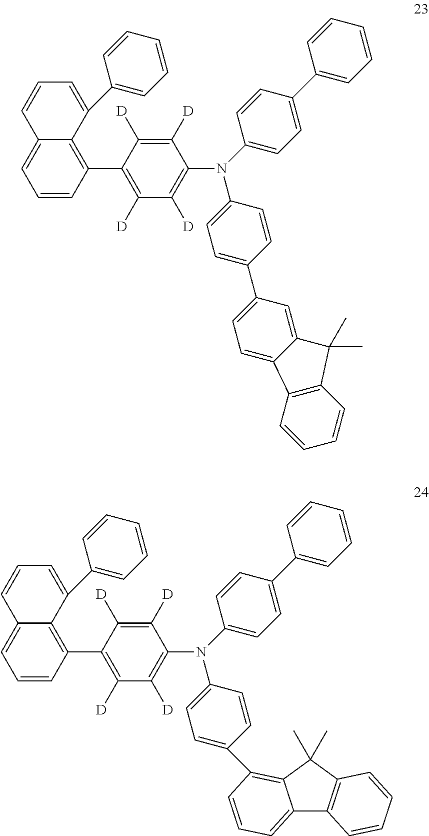 [SMILES: [2H]c1c([2H])c(N(c2ccc(-c3ccccc3)cc2)c2ccc(-c3ccc4c(c3)C(C)(C)c3ccccc3-4)cc2)c([2H])c([2H])c1-c1cccc2cccc(-c3ccccc3)c12 ; [2H]c1c([2H])c(N(c2ccc(-c3ccccc3)cc2)c2ccc(-c3cccc4c3C(C)(C)c3ccccc3-4)cc2)c([2H])c([2H])c1-c1cccc2cccc(-c3ccccc3)c12]

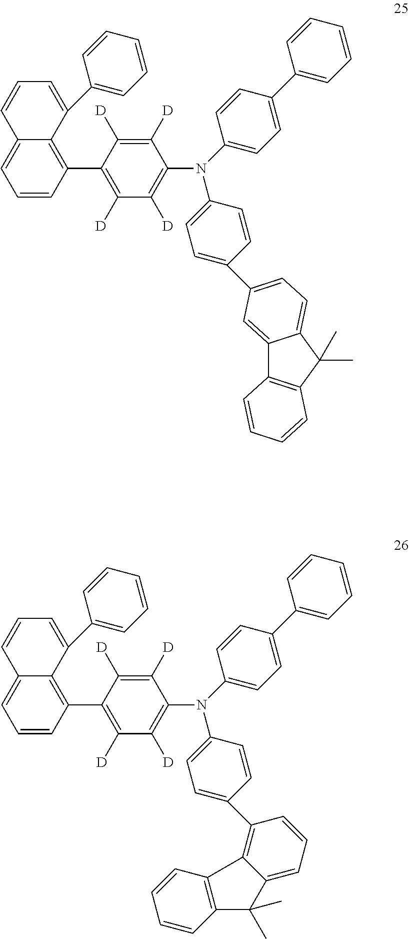 [SMILES: [2H]c1c([2H])c(N(c2ccc(-c3ccccc3)cc2)c2ccc(-c3ccc4c(c3)-c3ccccc3C4(C)C)cc2)c([2H])c([2H])c1-c1cccc2cccc(-c3ccccc3)c12 ; [2H]c1c([2H])c(N(c2ccc(-c3ccccc3)cc2)c2ccc(-c3cccc4c3-c3ccccc3C4(C)C)cc2)c([2H])c([2H])c1-c1cccc2cccc(-c3ccccc3)c12]

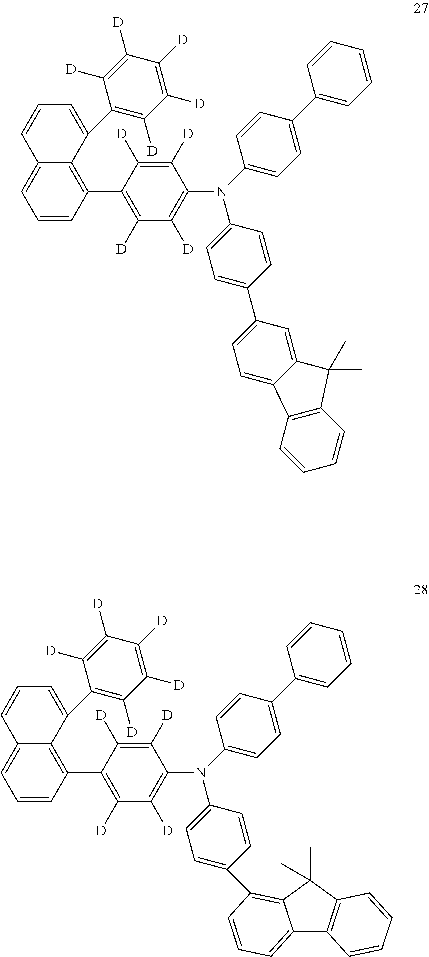 [SMILES: [2H]c1c([2H])c([2H])c(-c2cccc3cccc(-c4c([2H])c([2H])c(N(c5ccc(-c6ccccc6)cc5)c5ccc(-c6ccc7c(c6)C(C)(C)c6ccccc6-7)cc5)c([2H])c4[2H])c23)c([2H])c1[2H] ; [2H]c1c([2H])c([2H])c(-c2cccc3cccc(-c4c([2H])c([2H])c(N(c5ccc(-c6ccccc6)cc5)c5ccc(-c6cccc7c6C(C)(C)c6ccccc6-7)cc5)c([2H])c4[2H])c23)c([2H])c1[2H]]

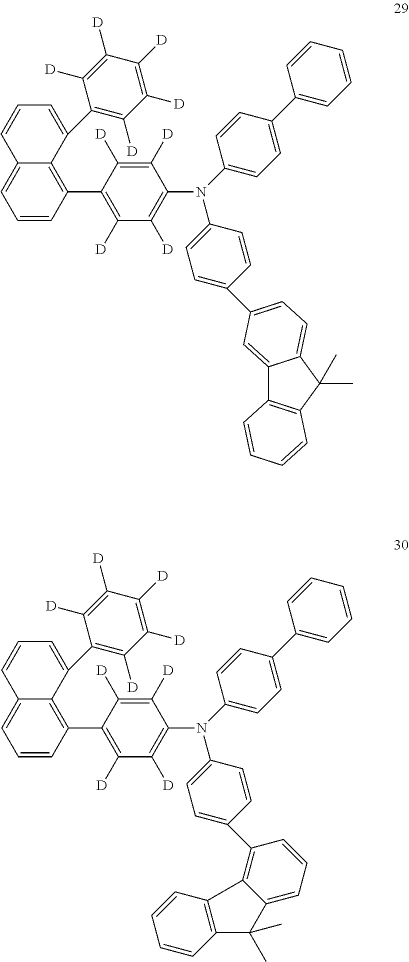 [SMILES: [2H]c1c([2H])c([2H])c(-c2cccc3cccc(-c4c([2H])c([2H])c(N(c5ccc(-c6ccccc6)cc5)c5ccc(-c6ccc7c(c6)-c6ccccc6C7(C)C)cc5)c([2H])c4[2H])c23)c([2H])c1[2H] ; [2H]c1c([2H])c([2H])c(-c2cccc3cccc(-c4c([2H])c([2H])c(N(c5ccc(-c6ccccc6)cc5)c5ccc(-c6cccc7c6-c6ccccc6C7(C)C)cc5)c([2H])c4[2H])c23)c([2H])c1[2H]]

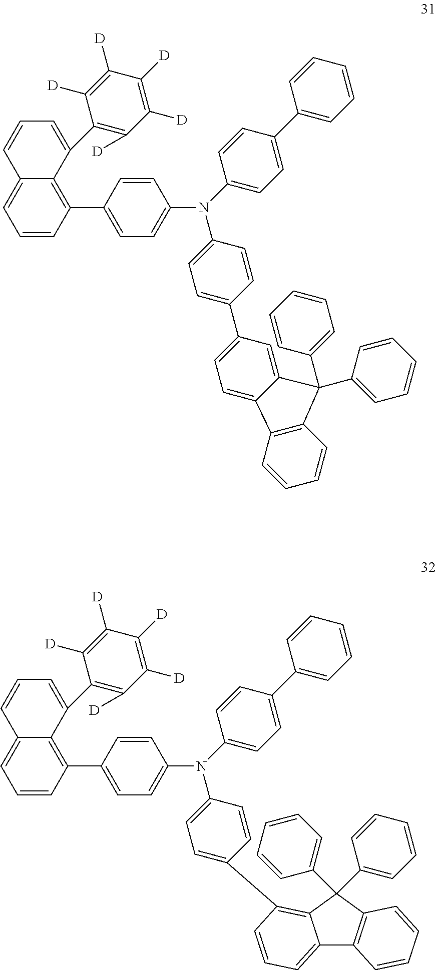 [SMILES: [2H]c1c([2H])c([2H])c(-c2cccc3cccc(-c4ccc(N(c5ccc(-c6ccccc6)cc5)c5ccc(-c6ccc7c(c6)C(c6ccccc6)(c6ccccc6)c6ccccc6-7)cc5)cc4)c23)c([2H])c1[2H] ; [2H]c1c([2H])c([2H])c(-c2cccc3cccc(-c4ccc(N(c5ccc(-c6ccccc6)cc5)c5ccc(-c6cccc7c6C(c6ccccc6)(c6ccccc6)c6ccccc6-7)cc5)cc4)c23)c([2H])c1[2H]]

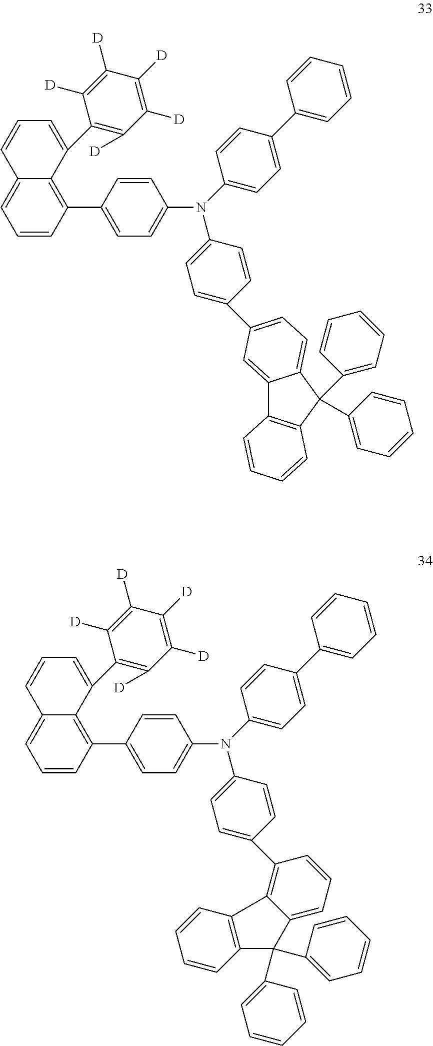 [SMILES: [2H]c1c([2H])c([2H])c(-c2cccc3cccc(-c4ccc(N(c5ccc(-c6ccccc6)cc5)c5ccc(-c6ccc7c(c6)-c6ccccc6C7(c6ccccc6)c6ccccc6)cc5)cc4)c23)c([2H])c1[2H] ; [2H]c1c([2H])c([2H])c(-c2cccc3cccc(-c4ccc(N(c5ccc(-c6ccccc6)cc5)c5ccc(-c6cccc7c6-c6ccccc6C7(c6ccccc6)c6ccccc6)cc5)cc4)c23)c([2H])c1[2H]]

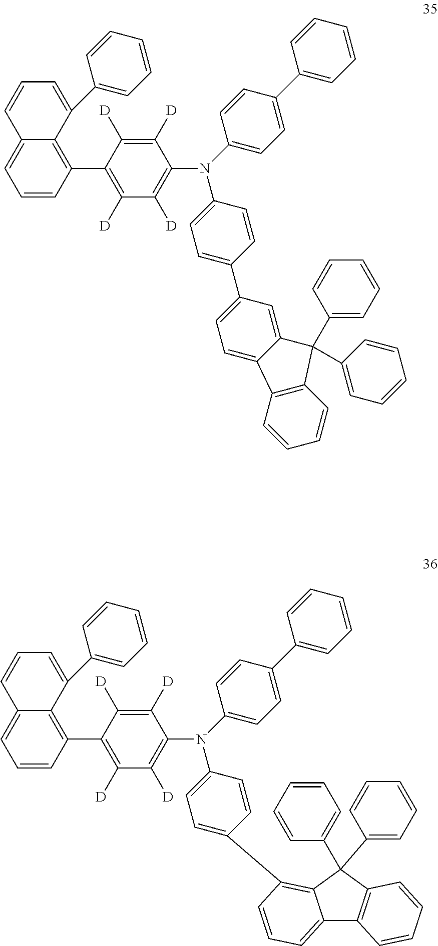 [SMILES: [2H]c1c([2H])c(N(c2ccc(-c3ccccc3)cc2)c2ccc(-c3ccc4c(c3)C(c3ccccc3)(c3ccccc3)c3ccccc3-4)cc2)c([2H])c([2H])c1-c1cccc2cccc(-c3ccccc3)c12 ; [2H]c1c([2H])c(N(c2ccc(-c3ccccc3)cc2)c2ccc(-c3cccc4c3C(c3ccccc3)(c3ccccc3)c3ccccc3-4)cc2)c([2H])c([2H])c1-c1cccc2cccc(-c3ccccc3)c12]

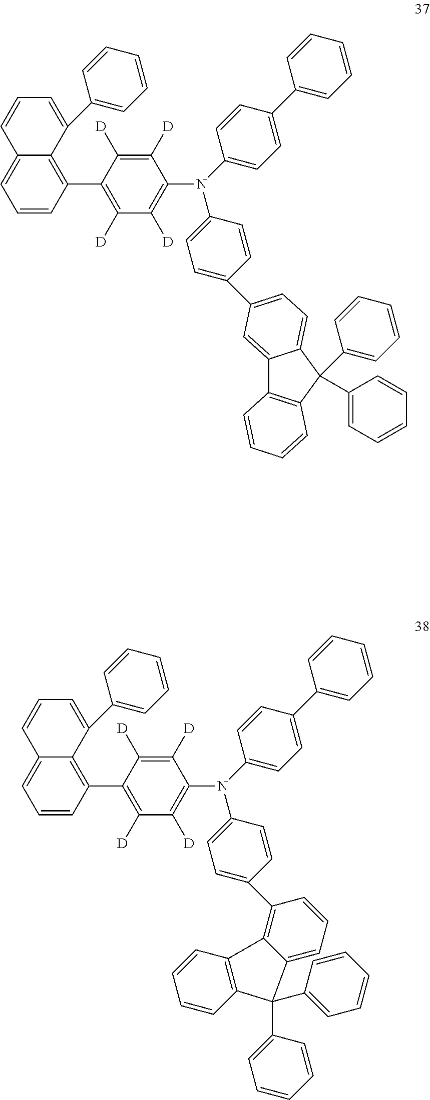 [SMILES: [2H]c1c([2H])c(N(c2ccc(-c3ccccc3)cc2)c2ccc(-c3ccc4c(c3)-c3ccccc3C4(c3ccccc3)c3ccccc3)cc2)c([2H])c([2H])c1-c1cccc2cccc(-c3ccccc3)c12 ; [2H]c1c([2H])c(N(c2ccc(-c3ccccc3)cc2)c2ccc(-c3cccc4c3-c3ccccc3C4(c3ccccc3)c3ccccc3)cc2)c([2H])c([2H])c1-c1cccc2cccc(-c3ccccc3)c12]

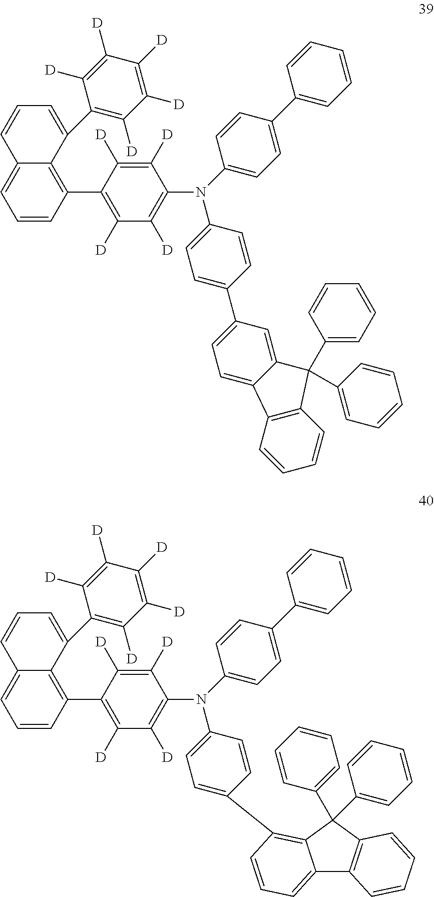 [SMILES: [2H]c1c([2H])c([2H])c(-c2cccc3cccc(-c4c([2H])c([2H])c(N(c5ccc(-c6ccccc6)cc5)c5ccc(-c6ccc7c(c6)C(c6ccccc6)(c6ccccc6)c6ccccc6-7)cc5)c([2H])c4[2H])c23)c([2H])c1[2H] ; [2H]c1c([2H])c([2H])c(-c2cccc3cccc(-c4c([2H])c([2H])c(N(c5ccc(-c6ccccc6)cc5)c5ccc(-c6cccc7c6C(c6ccccc6)(c6ccccc6)c6ccccc6-7)cc5)c([2H])c4[2H])c23)c([2H])c1[2H]]

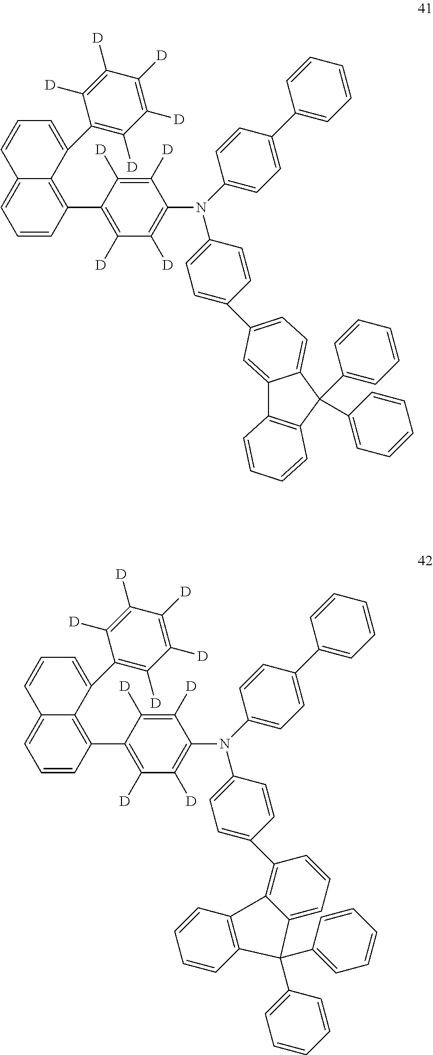 [SMILES: [2H]c1c([2H])c([2H])c(-c2cccc3cccc(-c4c([2H])c([2H])c(N(c5ccc(-c6ccccc6)cc5)c5ccc(-c6ccc7c(c6)-c6ccccc6C7(c6ccccc6)c6ccccc6)cc5)c([2H])c4[2H])c23)c([2H])c1[2H] ; [2H]c1c([2H])c([2H])c(-c2cccc3cccc(-c4c([2H])c([2H])c(N(c5ccc(-c6ccccc6)cc5)c5ccc(-c6cccc7c6-c6ccccc6C7(c6ccccc6)c6ccccc6)cc5)c([2H])c4[2H])c23)c([2H])c1[2H]]

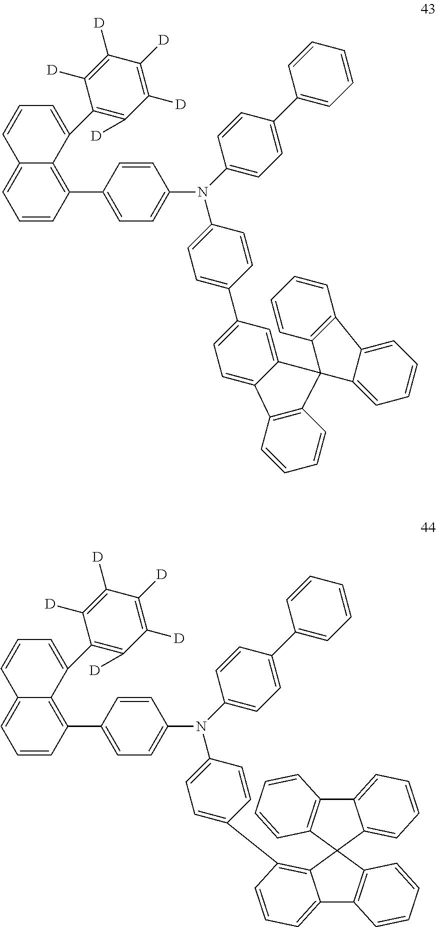 [SMILES: [2H]c1c([2H])c([2H])c(-c2cccc3cccc(-c4ccc(N(c5ccc(-c6ccccc6)cc5)c5ccc(-c6ccc7c(c6)C6(c8ccccc8-c8ccccc86)c6ccccc6-7)cc5)cc4)c23)c([2H])c1[2H] ; [2H]c1c([2H])c([2H])c(-c2cccc3cccc(-c4ccc(N(c5ccc(-c6ccccc6)cc5)c5ccc(-c6cccc7c6C6(c8ccccc8-c8ccccc86)c6ccccc6-7)cc5)cc4)c23)c([2H])c1[2H]]

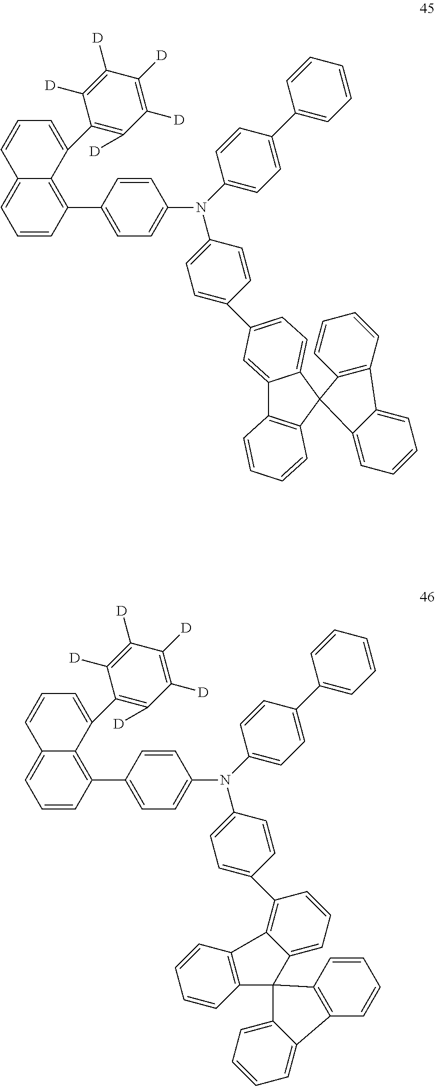 [SMILES: [2H]c1c([2H])c([2H])c(-c2cccc3cccc(-c4ccc(N(c5ccc(-c6ccccc6)cc5)c5ccc(-c6ccc7c(c6)-c6ccccc6C76c7ccccc7-c7ccccc76)cc5)cc4)c23)c([2H])c1[2H] ; [2H]c1c([2H])c([2H])c(-c2cccc3cccc(-c4ccc(N(c5ccc(-c6ccccc6)cc5)c5ccc(-c6cccc7c6-c6ccccc6C76c7ccccc7-c7ccccc76)cc5)cc4)c23)c([2H])c1[2H]]

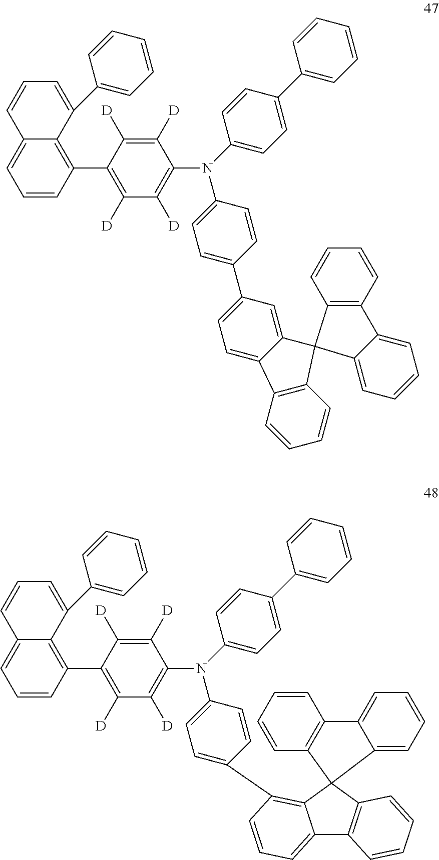 [SMILES: [2H]c1c([2H])c(N(c2ccc(-c3ccccc3)cc2)c2ccc(-c3ccc4c(c3)C3(c5ccccc5-c5ccccc53)c3ccccc3-4)cc2)c([2H])c([2H])c1-c1cccc2cccc(-c3ccccc3)c12 ; [2H]c1c([2H])c(N(c2ccc(-c3ccccc3)cc2)c2ccc(-c3cccc4c3C3(c5ccccc5-c5ccccc53)c3ccccc3-4)cc2)c([2H])c([2H])c1-c1cccc2cccc(-c3ccccc3)c12]

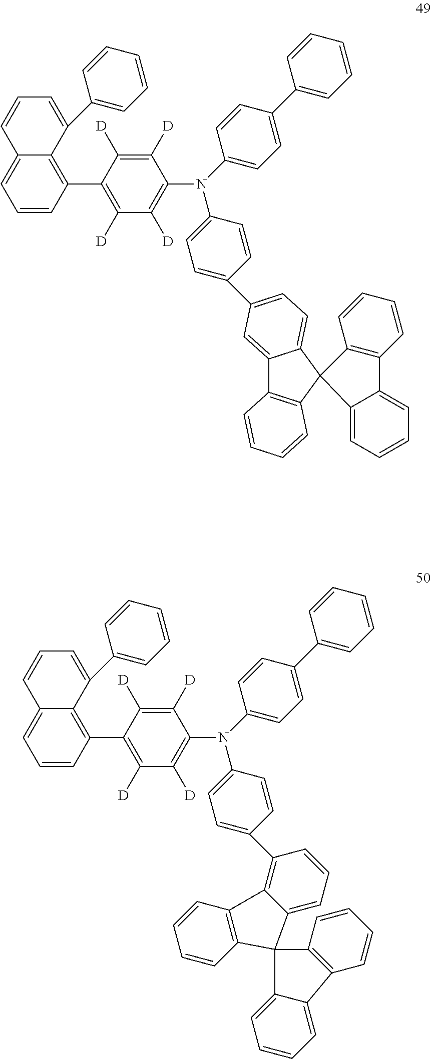 [SMILES: [2H]c1c([2H])c(N(c2ccc(-c3ccccc3)cc2)c2ccc(-c3ccc4c(c3)-c3ccccc3C43c4ccccc4-c4ccccc43)cc2)c([2H])c([2H])c1-c1cccc2cccc(-c3ccccc3)c12 ; [2H]c1c([2H])c(N(c2ccc(-c3ccccc3)cc2)c2ccc(-c3cccc4c3-c3ccccc3C43c4ccccc4-c4ccccc43)cc2)c([2H])c([2H])c1-c1cccc2cccc(-c3ccccc3)c12]

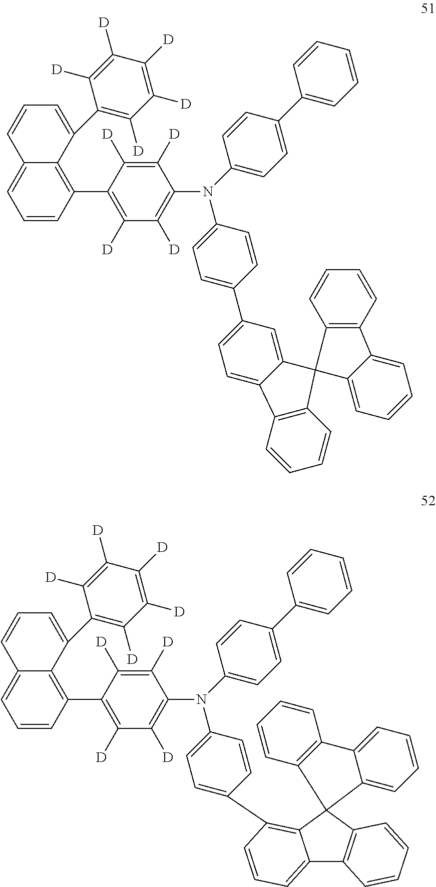 [SMILES: [2H]c1c([2H])c([2H])c(-c2cccc3cccc(-c4c([2H])c([2H])c(N(c5ccc(-c6ccccc6)cc5)c5ccc(-c6ccc7c(c6)C6(c8ccccc8-c8ccccc86)c6ccccc6-7)cc5)c([2H])c4[2H])c23)c([2H])c1[2H] ; [2H]c1c([2H])c([2H])c(-c2cccc3cccc(-c4c([2H])c([2H])c(N(c5ccc(-c6ccccc6)cc5)c5ccc(-c6cccc7c6C6(c8ccccc8-c8ccccc86)c6ccccc6-7)cc5)c([2H])c4[2H])c23)c([2H])c1[2H]]

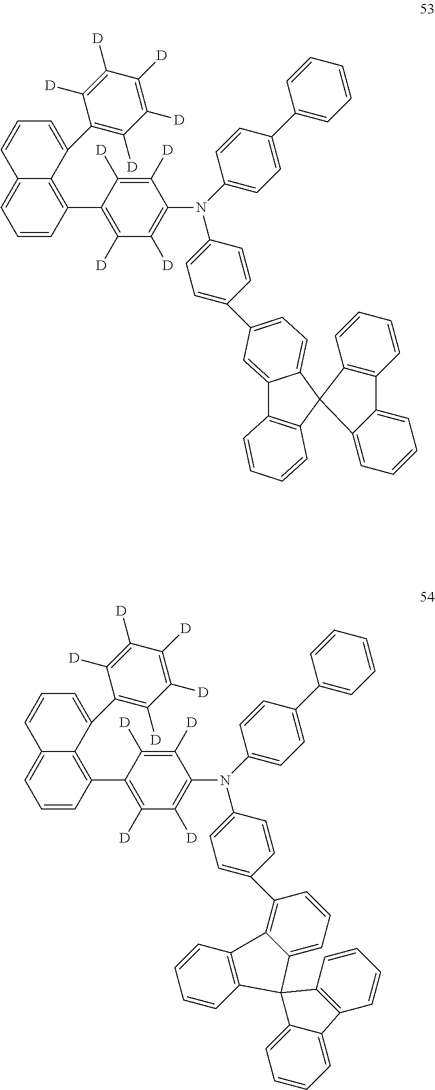 [SMILES: [2H]c1c([2H])c([2H])c(-c2cccc3cccc(-c4c([2H])c([2H])c(N(c5ccc(-c6ccccc6)cc5)c5ccc(-c6ccc7c(c6)-c6ccccc6C76c7ccccc7-c7ccccc76)cc5)c([2H])c4[2H])c23)c([2H])c1[2H] ; [2H]c1c([2H])c([2H])c(-c2cccc3cccc(-c4c([2H])c([2H])c(N(c5ccc(-c6ccccc6)cc5)c5ccc(-c6cccc7c6-c6ccccc6C76c7ccccc7-c7ccccc76)cc5)c([2H])c4[2H])c23)c([2H])c1[2H]]

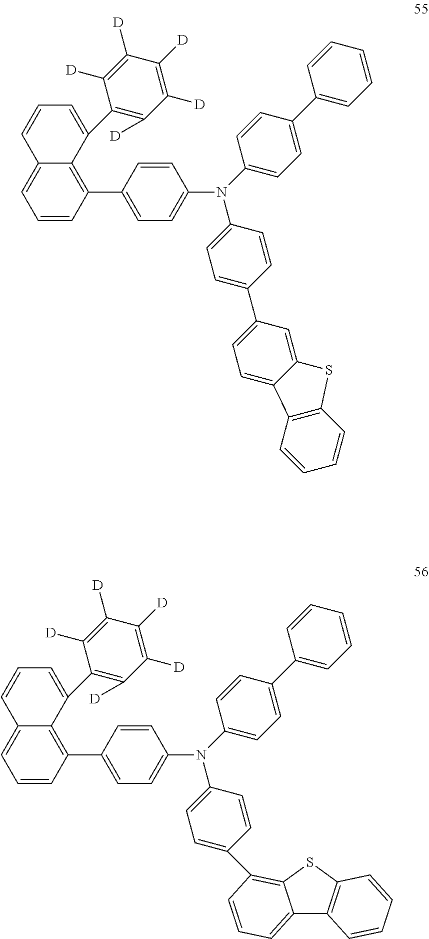 [SMILES: [2H]c1c([2H])c([2H])c(-c2cccc3cccc(-c4ccc(N(c5ccc(-c6ccccc6)cc5)c5ccc(-c6ccc7c(c6)sc6ccccc67)cc5)cc4)c23)c([2H])c1[2H] ; [2H]c1c([2H])c([2H])c(-c2cccc3cccc(-c4ccc(N(c5ccc(-c6ccccc6)cc5)c5ccc(-c6cccc7c6sc6ccccc67)cc5)cc4)c23)c([2H])c1[2H]]

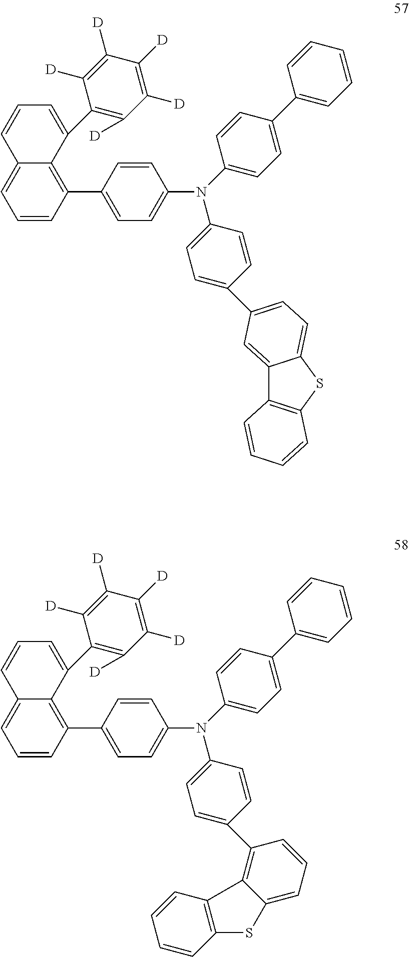 [SMILES: [2H]c1c([2H])c([2H])c(-c2cccc3cccc(-c4ccc(N(c5ccc(-c6ccccc6)cc5)c5ccc(-c6ccc7sc8ccccc8c7c6)cc5)cc4)c23)c([2H])c1[2H] ; [2H]c1c([2H])c([2H])c(-c2cccc3cccc(-c4ccc(N(c5ccc(-c6ccccc6)cc5)c5ccc(-c6cccc7sc8ccccc8c67)cc5)cc4)c23)c([2H])c1[2H]]

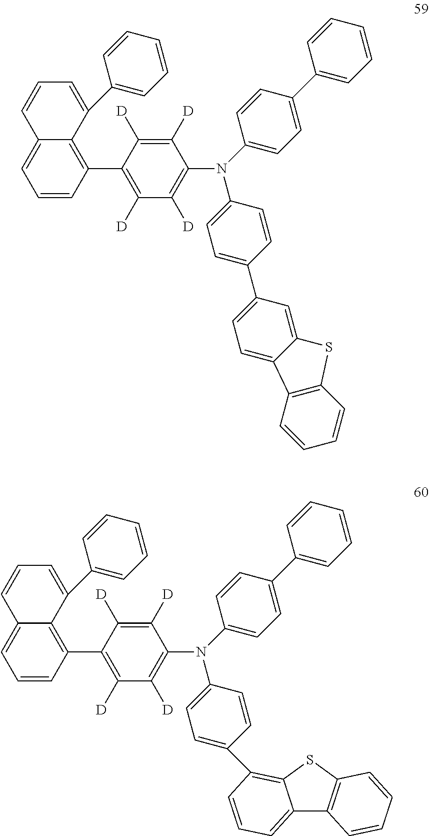 [SMILES: [2H]c1c([2H])c(N(c2ccc(-c3ccccc3)cc2)c2ccc(-c3ccc4c(c3)sc3ccccc34)cc2)c([2H])c([2H])c1-c1cccc2cccc(-c3ccccc3)c12 ; [2H]c1c([2H])c(N(c2ccc(-c3ccccc3)cc2)c2ccc(-c3cccc4c3sc3ccccc34)cc2)c([2H])c([2H])c1-c1cccc2cccc(-c3ccccc3)c12]

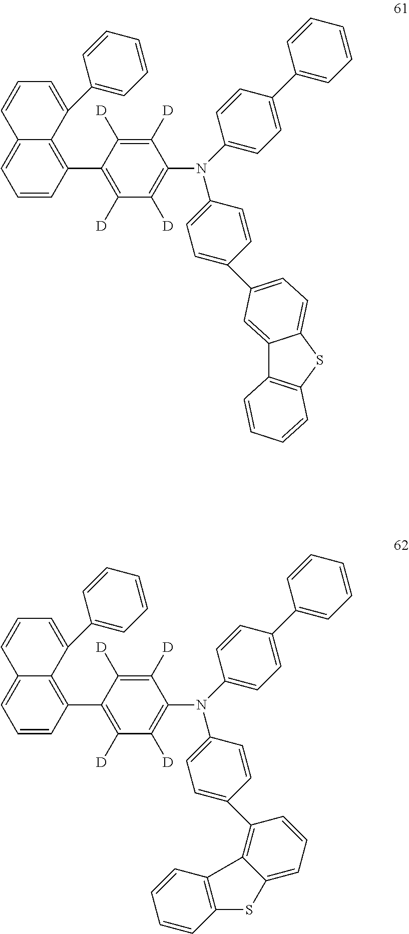 [SMILES: [2H]c1c([2H])c(N(c2ccc(-c3ccccc3)cc2)c2ccc(-c3ccc4sc5ccccc5c4c3)cc2)c([2H])c([2H])c1-c1cccc2cccc(-c3ccccc3)c12 ; [2H]c1c([2H])c(N(c2ccc(-c3ccccc3)cc2)c2ccc(-c3cccc4sc5ccccc5c34)cc2)c([2H])c([2H])c1-c1cccc2cccc(-c3ccccc3)c12]

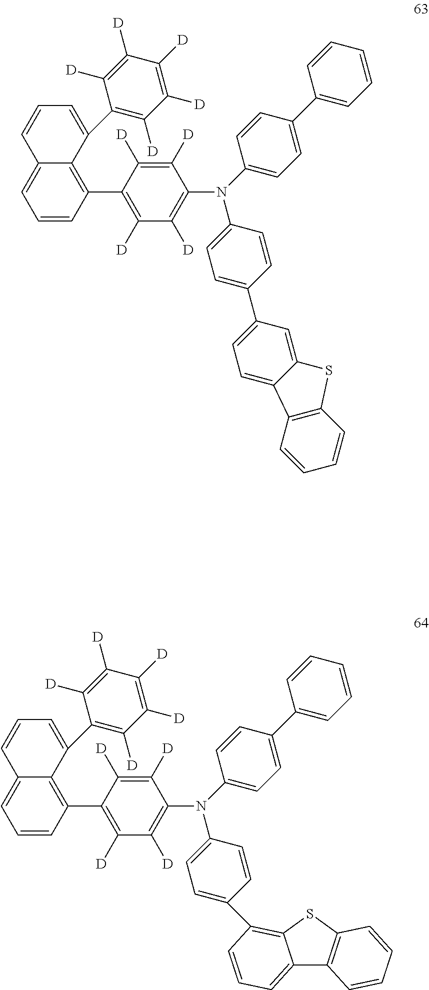 [SMILES: [2H]c1c([2H])c([2H])c(-c2cccc3cccc(-c4c([2H])c([2H])c(N(c5ccc(-c6ccccc6)cc5)c5ccc(-c6ccc7c(c6)sc6ccccc67)cc5)c([2H])c4[2H])c23)c([2H])c1[2H] ; [2H]c1c([2H])c([2H])c(-c2cccc3cccc(-c4c([2H])c([2H])c(N(c5ccc(-c6ccccc6)cc5)c5ccc(-c6cccc7c6sc6ccccc67)cc5)c([2H])c4[2H])c23)c([2H])c1[2H]]

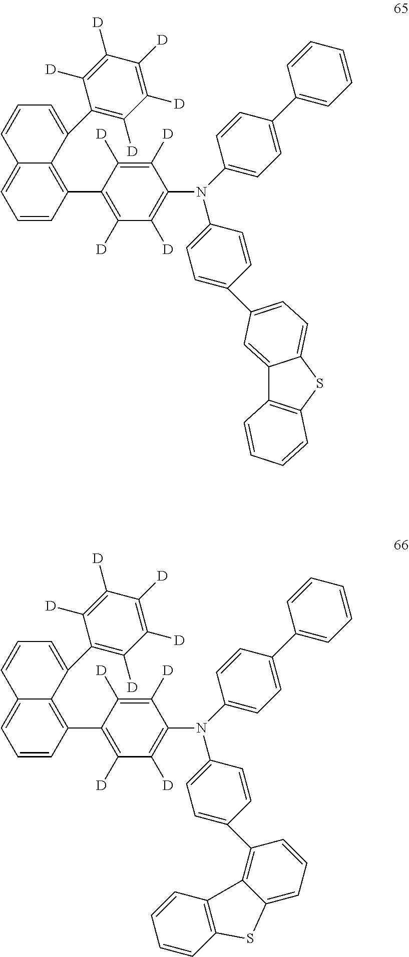 [SMILES: [2H]c1c([2H])c([2H])c(-c2cccc3cccc(-c4c([2H])c([2H])c(N(c5ccc(-c6ccccc6)cc5)c5ccc(-c6ccc7sc8ccccc8c7c6)cc5)c([2H])c4[2H])c23)c([2H])c1[2H] ; [2H]c1c([2H])c([2H])c(-c2cccc3cccc(-c4c([2H])c([2H])c(N(c5ccc(-c6ccccc6)cc5)c5ccc(-c6cccc7sc8ccccc8c67)cc5)c([2H])c4[2H])c23)c([2H])c1[2H]]

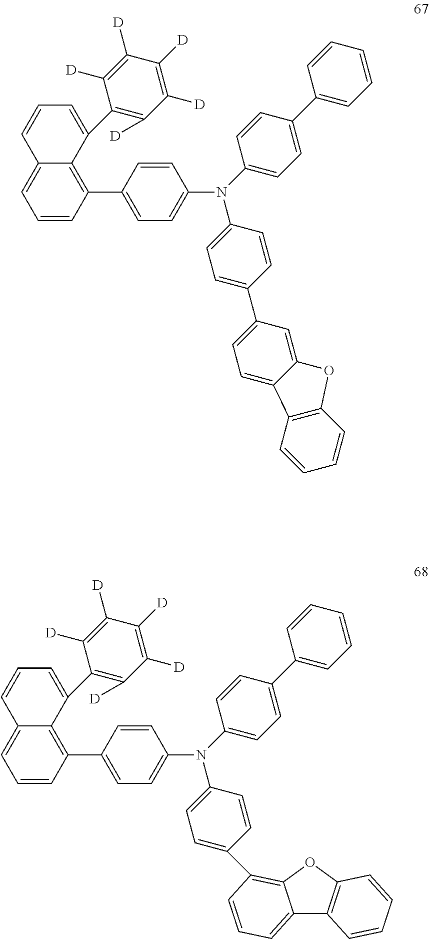 [SMILES: [2H]c1c([2H])c([2H])c(-c2cccc3cccc(-c4ccc(N(c5ccc(-c6ccccc6)cc5)c5ccc(-c6ccc7c(c6)oc6ccccc67)cc5)cc4)c23)c([2H])c1[2H] ; [2H]c1c([2H])c([2H])c(-c2cccc3cccc(-c4ccc(N(c5ccc(-c6ccccc6)cc5)c5ccc(-c6cccc7c6oc6ccccc67)cc5)cc4)c23)c([2H])c1[2H]]

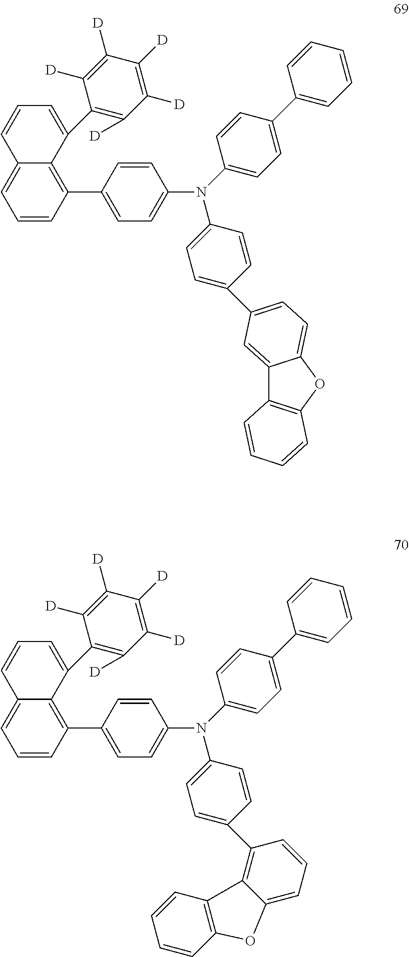 [SMILES: [2H]c1c([2H])c([2H])c(-c2cccc3cccc(-c4ccc(N(c5ccc(-c6ccccc6)cc5)c5ccc(-c6ccc7oc8ccccc8c7c6)cc5)cc4)c23)c([2H])c1[2H] ; [2H]c1c([2H])c([2H])c(-c2cccc3cccc(-c4ccc(N(c5ccc(-c6ccccc6)cc5)c5ccc(-c6cccc7oc8ccccc8c67)cc5)cc4)c23)c([2H])c1[2H]]

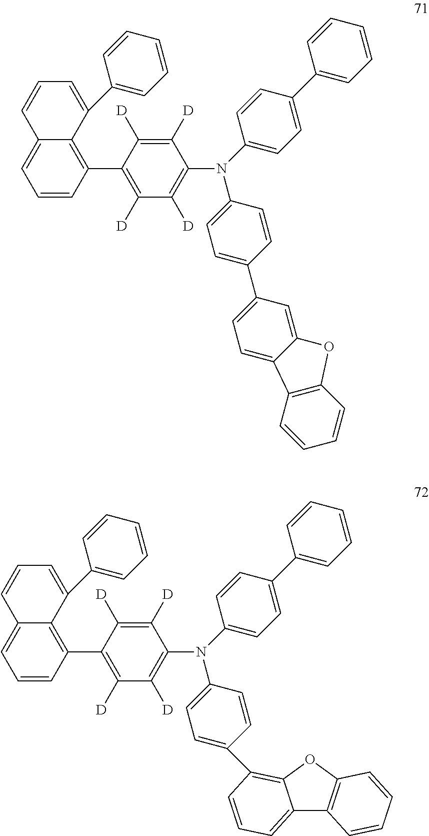 [SMILES: [2H]c1c([2H])c(N(c2ccc(-c3ccccc3)cc2)c2ccc(-c3ccc4c(c3)oc3ccccc34)cc2)c([2H])c([2H])c1-c1cccc2cccc(-c3ccccc3)c12 ; [2H]c1c([2H])c(N(c2ccc(-c3ccccc3)cc2)c2ccc(-c3cccc4c3oc3ccccc34)cc2)c([2H])c([2H])c1-c1cccc2cccc(-c3ccccc3)c12]

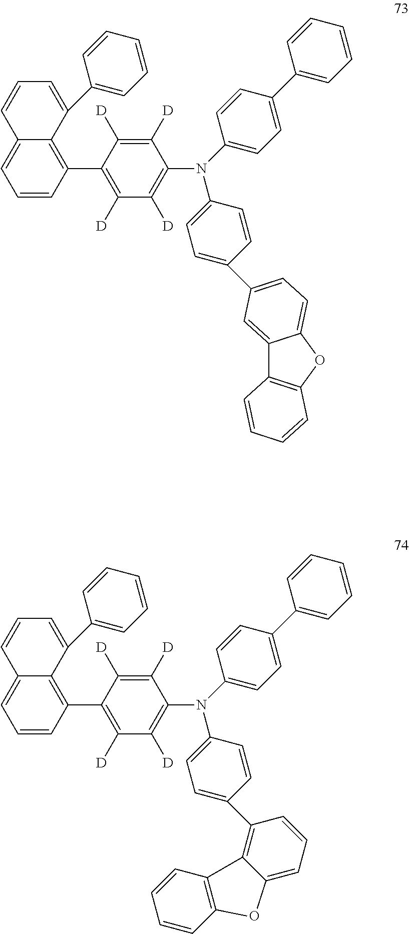 [SMILES: [2H]c1c([2H])c(N(c2ccc(-c3ccccc3)cc2)c2ccc(-c3ccc4oc5ccccc5c4c3)cc2)c([2H])c([2H])c1-c1cccc2cccc(-c3ccccc3)c12 ; [2H]c1c([2H])c(N(c2ccc(-c3ccccc3)cc2)c2ccc(-c3cccc4oc5ccccc5c34)cc2)c([2H])c([2H])c1-c1cccc2cccc(-c3ccccc3)c12]

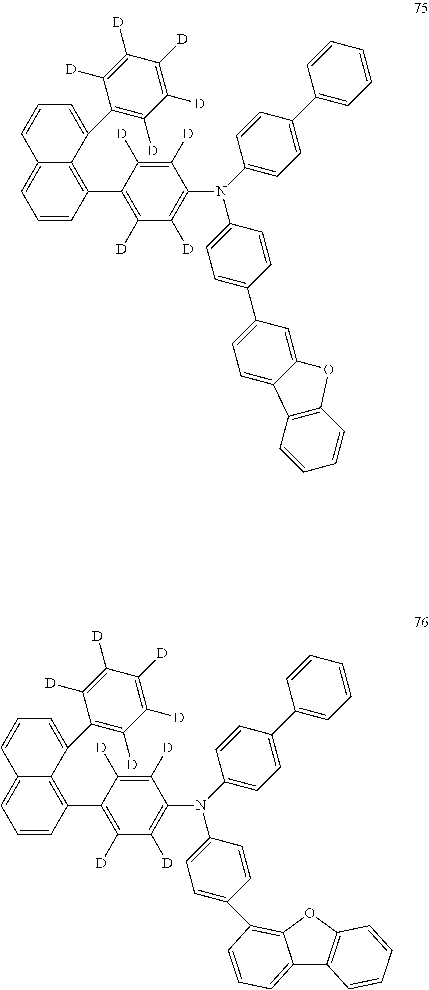 [SMILES: [2H]c1c([2H])c([2H])c(-c2cccc3cccc(-c4c([2H])c([2H])c(N(c5ccc(-c6ccccc6)cc5)c5ccc(-c6ccc7c(c6)oc6ccccc67)cc5)c([2H])c4[2H])c23)c([2H])c1[2H] ; [2H]c1c([2H])c([2H])c(-c2cccc3cccc(-c4c([2H])c([2H])c(N(c5ccc(-c6ccccc6)cc5)c5ccc(-c6cccc7c6oc6ccccc67)cc5)c([2H])c4[2H])c23)c([2H])c1[2H]]

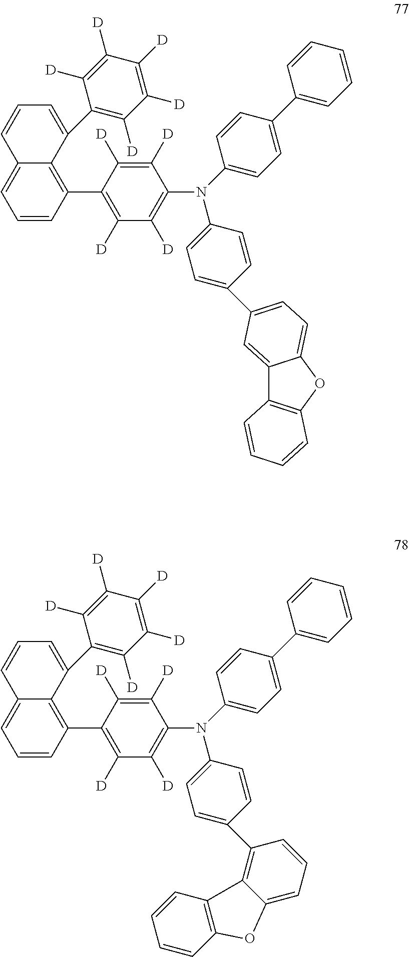 [SMILES: [2H]c1c([2H])c([2H])c(-c2cccc3cccc(-c4c([2H])c([2H])c(N(c5ccc(-c6ccccc6)cc5)c5ccc(-c6ccc7oc8ccccc8c7c6)cc5)c([2H])c4[2H])c23)c([2H])c1[2H] ; [2H]c1c([2H])c([2H])c(-c2cccc3cccc(-c4c([2H])c([2H])c(N(c5ccc(-c6ccccc6)cc5)c5ccc(-c6cccc7oc8ccccc8c67)cc5)c([2H])c4[2H])c23)c([2H])c1[2H]]

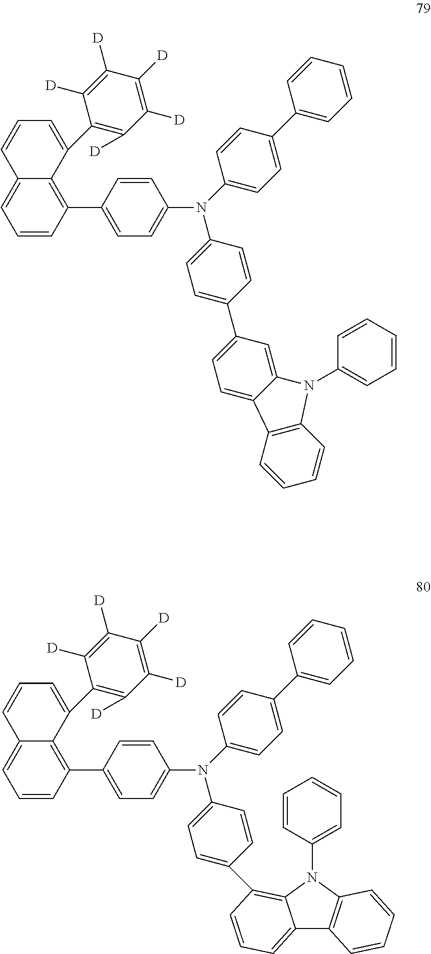 [SMILES: [2H]c1c([2H])c([2H])c(-c2cccc3cccc(-c4ccc(N(c5ccc(-c6ccccc6)cc5)c5ccc(-c6ccc7c8ccccc8n(-c8ccccc8)c7c6)cc5)cc4)c23)c([2H])c1[2H] ; [2H]c1c([2H])c([2H])c(-c2cccc3cccc(-c4ccc(N(c5ccc(-c6ccccc6)cc5)c5ccc(-c6cccc7c8ccccc8n(-c8ccccc8)c67)cc5)cc4)c23)c([2H])c1[2H]]

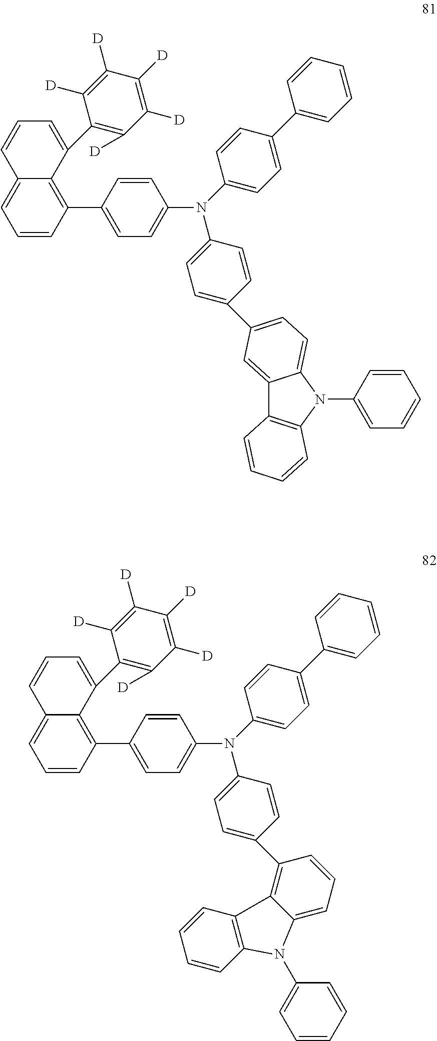 [SMILES: [2H]c1c([2H])c([2H])c(-c2cccc3cccc(-c4ccc(N(c5ccc(-c6ccccc6)cc5)c5ccc(-c6ccc7c(c6)c6ccccc6n7-c6ccccc6)cc5)cc4)c23)c([2H])c1[2H] ; [2H]c1c([2H])c([2H])c(-c2cccc3cccc(-c4ccc(N(c5ccc(-c6ccccc6)cc5)c5ccc(-c6cccc7c6c6ccccc6n7-c6ccccc6)cc5)cc4)c23)c([2H])c1[2H]]

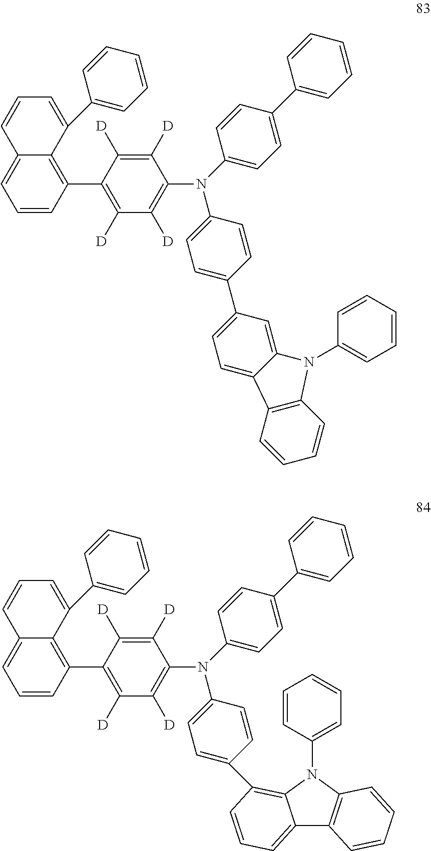 [SMILES: [2H]c1c([2H])c(N(c2ccc(-c3ccccc3)cc2)c2ccc(-c3ccc4c5ccccc5n(-c5ccccc5)c4c3)cc2)c([2H])c([2H])c1-c1cccc2cccc(-c3ccccc3)c12 ; [2H]c1c([2H])c(N(c2ccc(-c3ccccc3)cc2)c2ccc(-c3cccc4c5ccccc5n(-c5ccccc5)c34)cc2)c([2H])c([2H])c1-c1cccc2cccc(-c3ccccc3)c12]

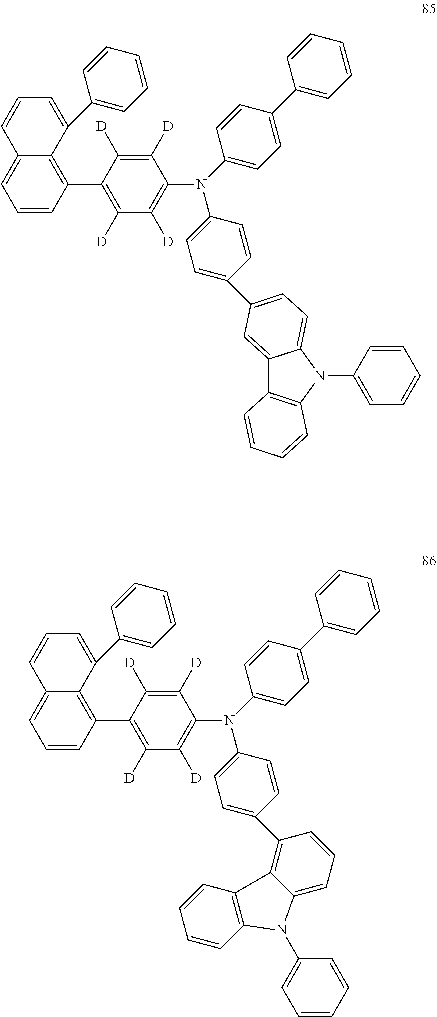 [SMILES: [2H]c1c([2H])c(N(c2ccc(-c3ccccc3)cc2)c2ccc(-c3ccc4c(c3)c3ccccc3n4-c3ccccc3)cc2)c([2H])c([2H])c1-c1cccc2cccc(-c3ccccc3)c12 ; [2H]c1c([2H])c(N(c2ccc(-c3ccccc3)cc2)c2ccc(-c3cccc4c3c3ccccc3n4-c3ccccc3)cc2)c([2H])c([2H])c1-c1cccc2cccc(-c3ccccc3)c12]

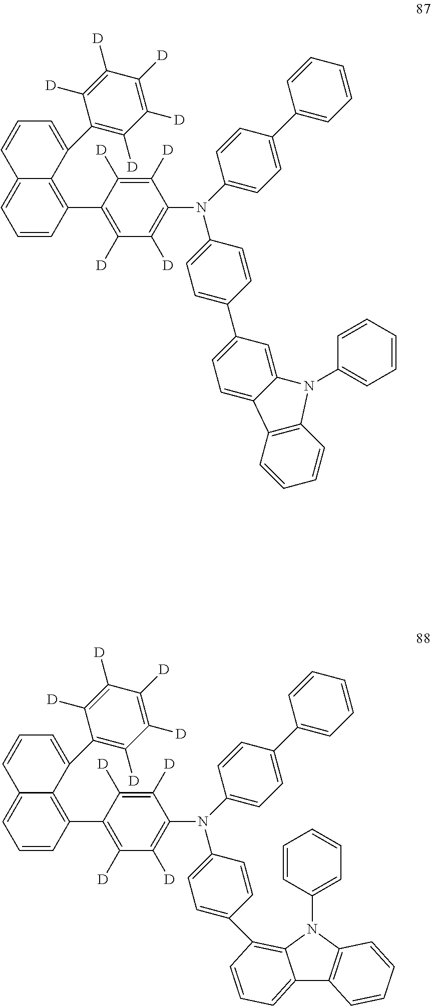 [SMILES: [2H]c1c([2H])c([2H])c(-c2cccc3cccc(-c4c([2H])c([2H])c(N(c5ccc(-c6ccccc6)cc5)c5ccc(-c6ccc7c8ccccc8n(-c8ccccc8)c7c6)cc5)c([2H])c4[2H])c23)c([2H])c1[2H] ; [2H]c1c([2H])c([2H])c(-c2cccc3cccc(-c4c([2H])c([2H])c(N(c5ccc(-c6ccccc6)cc5)c5ccc(-c6cccc7c8ccccc8n(-c8ccccc8)c67)cc5)c([2H])c4[2H])c23)c([2H])c1[2H]]

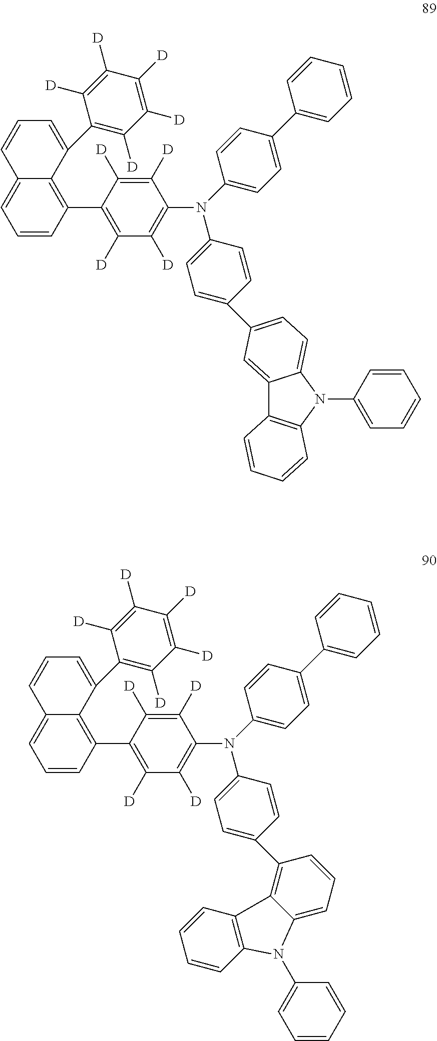 [SMILES: [2H]c1c([2H])c([2H])c(-c2cccc3cccc(-c4c([2H])c([2H])c(N(c5ccc(-c6ccccc6)cc5)c5ccc(-c6ccc7c(c6)c6ccccc6n7-c6ccccc6)cc5)c([2H])c4[2H])c23)c([2H])c1[2H] ; [2H]c1c([2H])c([2H])c(-c2cccc3cccc(-c4c([2H])c([2H])c(N(c5ccc(-c6ccccc6)cc5)c5ccc(-c6cccc7c6c6ccccc6n7-c6ccccc6)cc5)c([2H])c4[2H])c23)c([2H])c1[2H]]

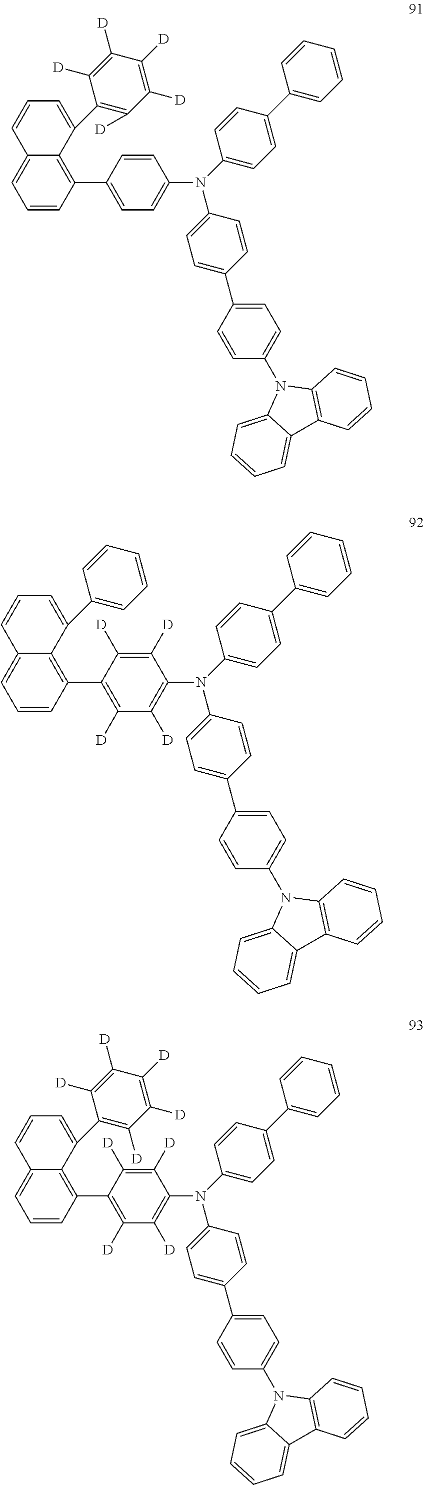 [SMILES: [2H]c1c([2H])c([2H])c(-c2cccc3cccc(-c4ccc(N(c5ccc(-c6ccccc6)cc5)c5ccc(-c6ccc(-n7c8ccccc8c8ccccc87)cc6)cc5)cc4)c23)c([2H])c1[2H] ; [2H]c1c([2H])c(N(c2ccc(-c3ccccc3)cc2)c2ccc(-c3ccc(-n4c5ccccc5c5ccccc54)cc3)cc2)c([2H])c([2H])c1-c1cccc2cccc(-c3ccccc3)c12 ; [2H]c1c([2H])c([2H])c(-c2cccc3cccc(-c4c([2H])c([2H])c(N(c5ccc(-c6ccccc6)cc5)c5ccc(-c6ccc(-n7c8ccccc8c8ccccc87)cc6)cc5)c([2H])c4[2H])c23)c([2H])c1[2H]]

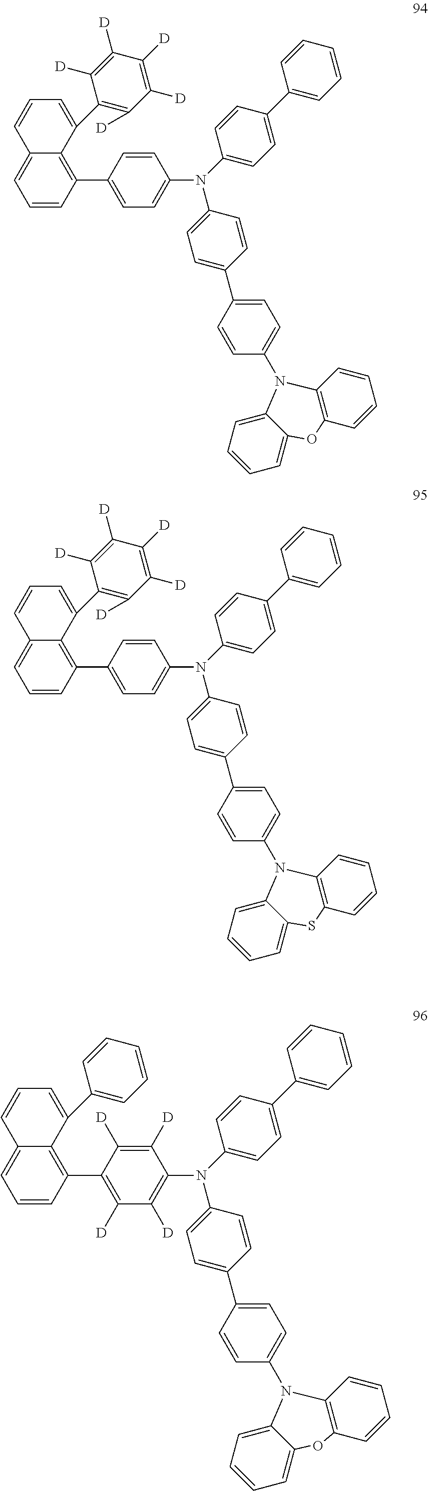 [SMILES: [2H]c1c([2H])c([2H])c(-c2cccc3cccc(-c4ccc(N(c5ccc(-c6ccccc6)cc5)c5ccc(-c6ccc(N7c8ccccc8Oc8ccccc87)cc6)cc5)cc4)c23)c([2H])c1[2H] ; [2H]c1c([2H])c([2H])c(-c2cccc3cccc(-c4ccc(N(c5ccc(-c6ccccc6)cc5)c5ccc(-c6ccc(N7c8ccccc8Sc8ccccc87)cc6)cc5)cc4)c23)c([2H])c1[2H] ; [2H]c1c([2H])c(N(c2ccc(-c3ccccc3)cc2)c2ccc(-c3ccc(N4c5ccccc5Oc5ccccc54)cc3)cc2)c([2H])c([2H])c1-c1cccc2cccc(-c3ccccc3)c12]

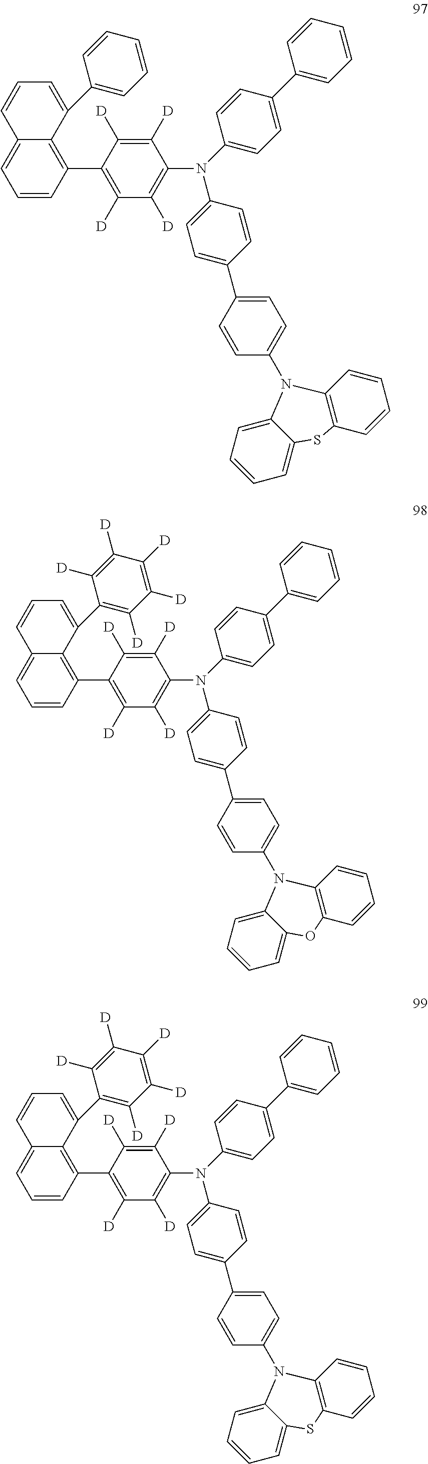 [SMILES: [2H]c1c([2H])c(N(c2ccc(-c3ccccc3)cc2)c2ccc(-c3ccc(N4c5ccccc5Sc5ccccc54)cc3)cc2)c([2H])c([2H])c1-c1cccc2cccc(-c3ccccc3)c12 ; [2H]c1c([2H])c([2H])c(-c2cccc3cccc(-c4c([2H])c([2H])c(N(c5ccc(-c6ccccc6)cc5)c5ccc(-c6ccc(N7c8ccccc8Oc8ccccc87)cc6)cc5)c([2H])c4[2H])c23)c([2H])c1[2H] ; [2H]c1c([2H])c([2H])c(-c2cccc3cccc(-c4c([2H])c([2H])c(N(c5ccc(-c6ccccc6)cc5)c5ccc(-c6ccc(N7c8ccccc8Sc8ccccc87)cc6)cc5)c([2H])c4[2H])c23)c([2H])c1[2H]]

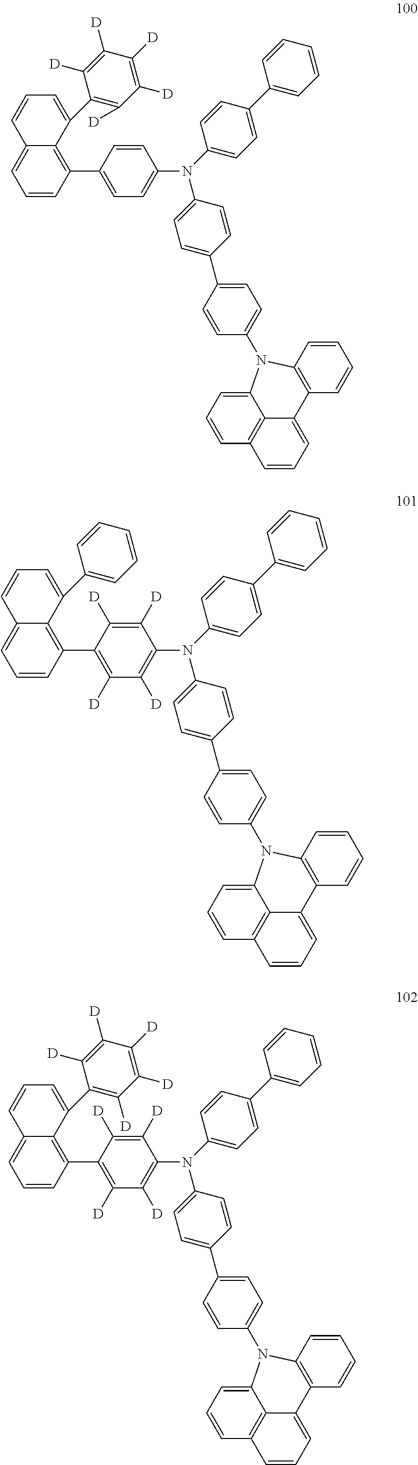 [SMILES: [2H]c1c([2H])c([2H])c(-c2cccc3cccc(-c4ccc(N(c5ccc(-c6ccccc6)cc5)c5ccc(-c6ccc(N7c8ccccc8-c8cccc9cccc7c89)cc6)cc5)cc4)c23)c([2H])c1[2H] ; [2H]c1c([2H])c(N(c2ccc(-c3ccccc3)cc2)c2ccc(-c3ccc(N4c5ccccc5-c5cccc6cccc4c56)cc3)cc2)c([2H])c([2H])c1-c1cccc2cccc(-c3ccccc3)c12 ; [2H]c1c([2H])c([2H])c(-c2cccc3cccc(-c4c([2H])c([2H])c(N(c5ccc(-c6ccccc6)cc5)c5ccc(-c6ccc(N7c8ccccc8-c8cccc9cccc7c89)cc6)cc5)c([2H])c4[2H])c23)c([2H])c1[2H]]

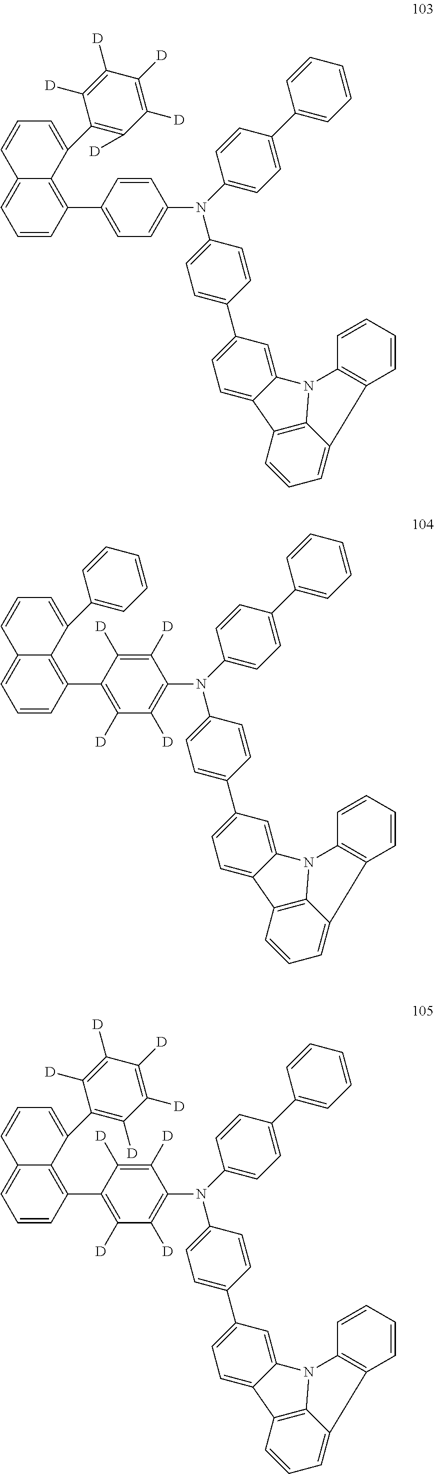 [SMILES: [2H]c1c([2H])c([2H])c(-c2cccc3cccc(-c4ccc(N(c5ccc(-c6ccccc6)cc5)c5ccc(-c6ccc7c8cccc9c%10ccccc%10n(c7c6)c98)cc5)cc4)c23)c([2H])c1[2H] ; [2H]c1c([2H])c(N(c2ccc(-c3ccccc3)cc2)c2ccc(-c3ccc4c5cccc6c7ccccc7n(c4c3)c65)cc2)c([2H])c([2H])c1-c1cccc2cccc(-c3ccccc3)c12 ; [2H]c1c([2H])c([2H])c(-c2cccc3cccc(-c4c([2H])c([2H])c(N(c5ccc(-c6ccccc6)cc5)c5ccc(-c6ccc7c8cccc9c%10ccccc%10n(c7c6)c98)cc5)c([2H])c4[2H])c23)c([2H])c1[2H]]

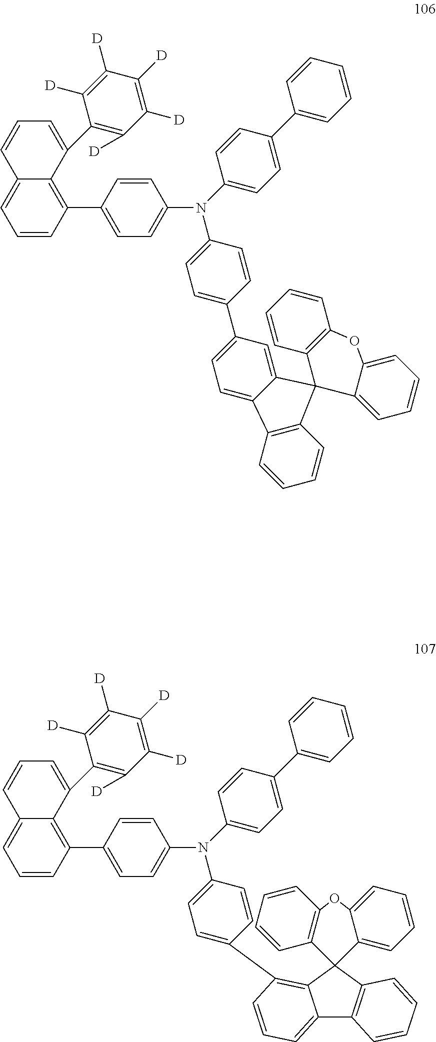 [SMILES: [2H]c1c([2H])c([2H])c(-c2cccc3cccc(-c4ccc(N(c5ccc(-c6ccccc6)cc5)c5ccc(-c6ccc7c(c6)C6(c8ccccc8Oc8ccccc86)c6ccccc6-7)cc5)cc4)c23)c([2H])c1[2H] ; [2H]c1c([2H])c([2H])c(-c2cccc3cccc(-c4ccc(N(c5ccc(-c6ccccc6)cc5)c5ccc(-c6cccc7c6C6(c8ccccc8Oc8ccccc86)c6ccccc6-7)cc5)cc4)c23)c([2H])c1[2H]]

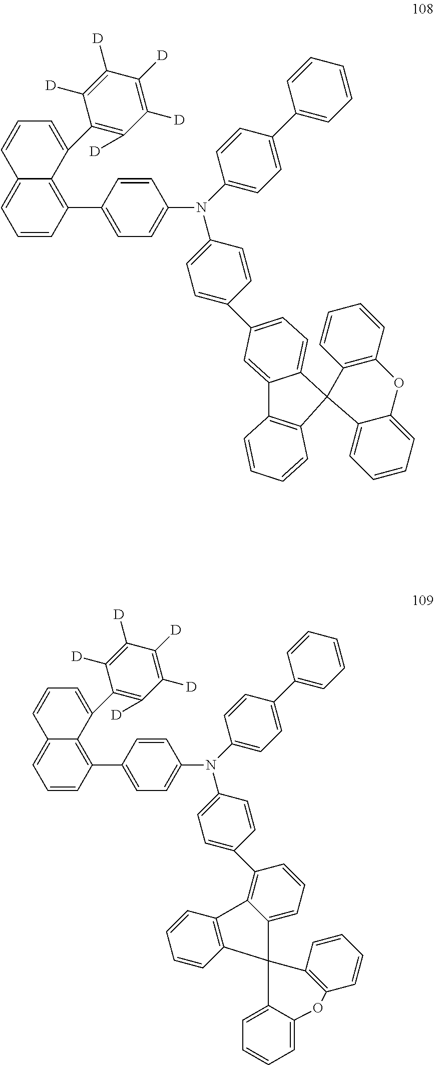 [SMILES: [2H]c1c([2H])c([2H])c(-c2cccc3cccc(-c4ccc(N(c5ccc(-c6ccccc6)cc5)c5ccc(-c6ccc7c(c6)-c6ccccc6C76c7ccccc7Oc7ccccc76)cc5)cc4)c23)c([2H])c1[2H] ; [2H]c1c([2H])c([2H])c(-c2cccc3cccc(-c4ccc(N(c5ccc(-c6ccccc6)cc5)c5ccc(-c6cccc7c6-c6ccccc6C76c7ccccc7Oc7ccccc76)cc5)cc4)c23)c([2H])c1[2H]]

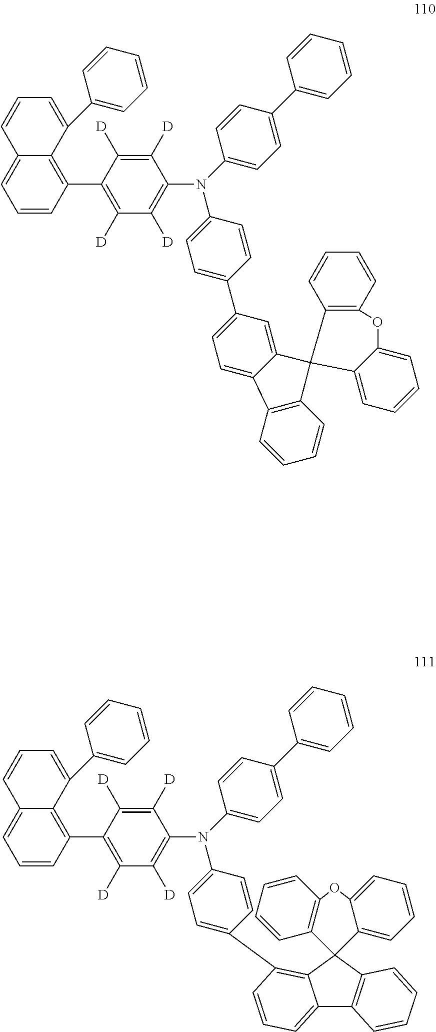 [SMILES: [2H]c1c([2H])c(N(c2ccc(-c3ccccc3)cc2)c2ccc(-c3ccc4c(c3)C3(c5ccccc5Oc5ccccc53)c3ccccc3-4)cc2)c([2H])c([2H])c1-c1cccc2cccc(-c3ccccc3)c12 ; [2H]c1c([2H])c(N(c2ccc(-c3ccccc3)cc2)c2ccc(-c3cccc4c3C3(c5ccccc5Oc5ccccc53)c3ccccc3-4)cc2)c([2H])c([2H])c1-c1cccc2cccc(-c3ccccc3)c12]

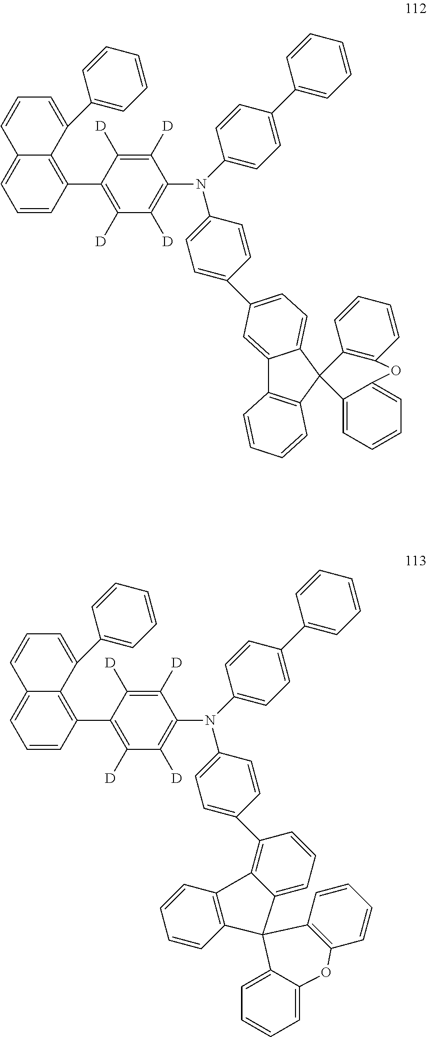 [SMILES: [2H]c1c([2H])c(N(c2ccc(-c3ccccc3)cc2)c2ccc(-c3ccc4c(c3)-c3ccccc3C43c4ccccc4Oc4ccccc43)cc2)c([2H])c([2H])c1-c1cccc2cccc(-c3ccccc3)c12 ; [2H]c1c([2H])c(N(c2ccc(-c3ccccc3)cc2)c2ccc(-c3cccc4c3-c3ccccc3C43c4ccccc4Oc4ccccc43)cc2)c([2H])c([2H])c1-c1cccc2cccc(-c3ccccc3)c12]

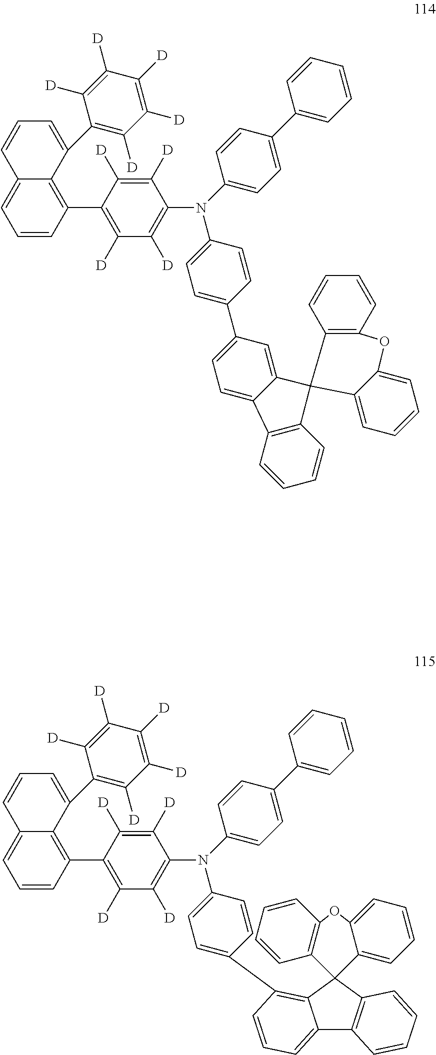 [SMILES: [2H]c1c([2H])c([2H])c(-c2cccc3cccc(-c4c([2H])c([2H])c(N(c5ccc(-c6ccccc6)cc5)c5ccc(-c6ccc7c(c6)C6(c8ccccc8Oc8ccccc86)c6ccccc6-7)cc5)c([2H])c4[2H])c23)c([2H])c1[2H] ; [2H]c1c([2H])c([2H])c(-c2cccc3cccc(-c4c([2H])c([2H])c(N(c5ccc(-c6ccccc6)cc5)c5ccc(-c6cccc7c6C6(c8ccccc8Oc8ccccc86)c6ccccc6-7)cc5)c([2H])c4[2H])c23)c([2H])c1[2H]]

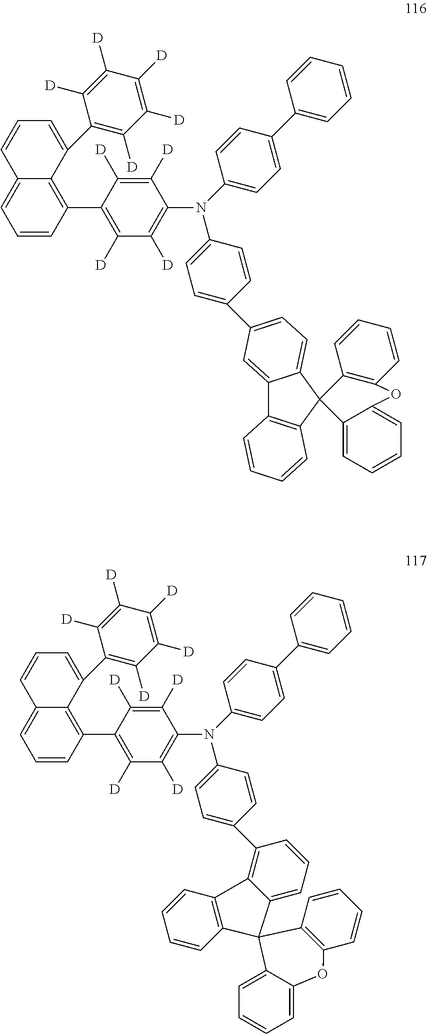 [SMILES: [2H]c1c([2H])c([2H])c(-c2cccc3cccc(-c4c([2H])c([2H])c(N(c5ccc(-c6ccccc6)cc5)c5ccc(-c6ccc7c(c6)-c6ccccc6C76c7ccccc7Oc7ccccc76)cc5)c([2H])c4[2H])c23)c([2H])c1[2H] ; [2H]c1c([2H])c([2H])c(-c2cccc3cccc(-c4c([2H])c([2H])c(N(c5ccc(-c6ccccc6)cc5)c5ccc(-c6cccc7c6-c6ccccc6C76c7ccccc7Oc7ccccc76)cc5)c([2H])c4[2H])c23)c([2H])c1[2H]]

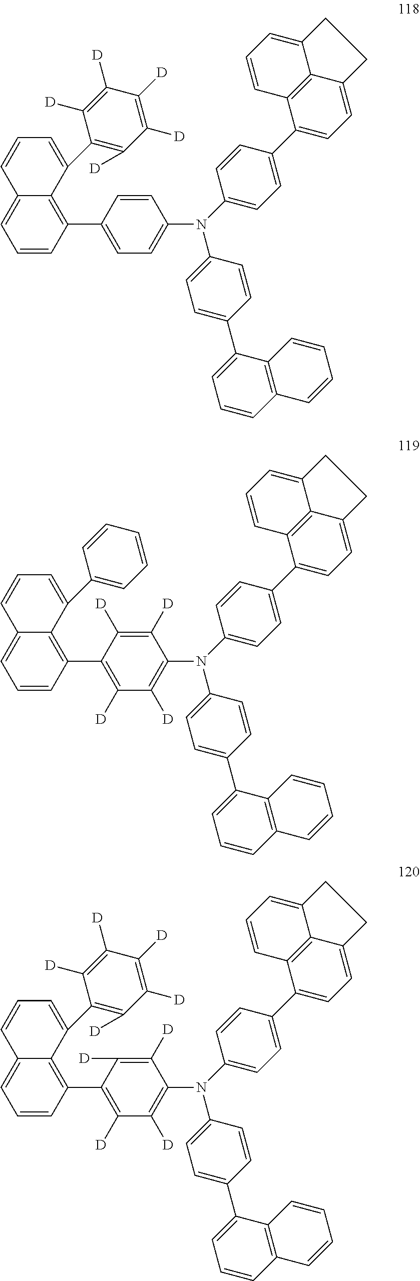

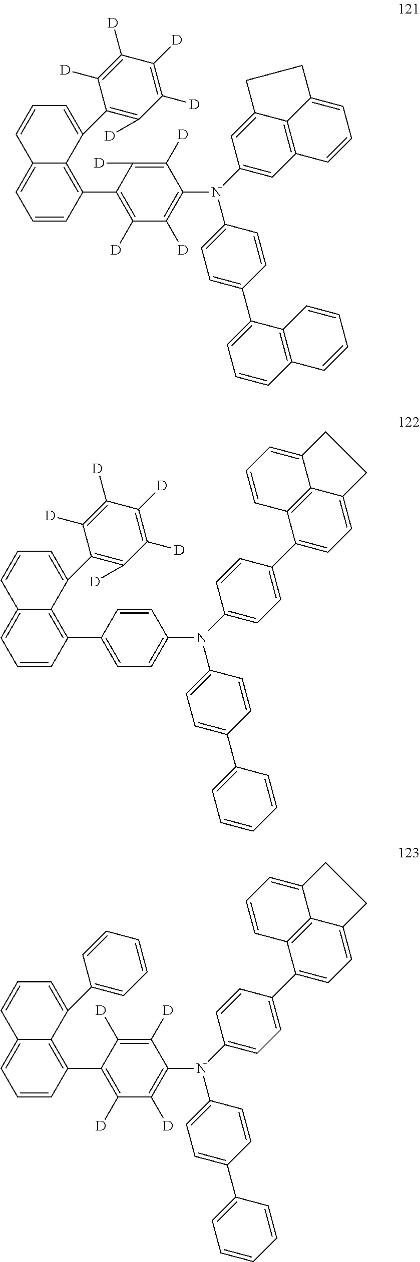 [SMILES: [2H]c1c([2H])c([2H])c(-c2cccc3cccc(-c4c([2H])c([2H])c(N(c5ccc(-c6cccc7ccccc67)cc5)c5cc6c7c(cccc7c5)CC6)c([2H])c4[2H])c23)c([2H])c1[2H] ; [2H]c1c([2H])c([2H])c(-c2cccc3cccc(-c4ccc(N(c5ccc(-c6ccccc6)cc5)c5ccc(-c6ccc7c8c(cccc68)CC7)cc5)cc4)c23)c([2H])c1[2H] ; [2H]c1c([2H])c(N(c2ccc(-c3ccccc3)cc2)c2ccc(-c3ccc4c5c(cccc35)CC4)cc2)c([2H])c([2H])c1-c1cccc2cccc(-c3ccccc3)c12]

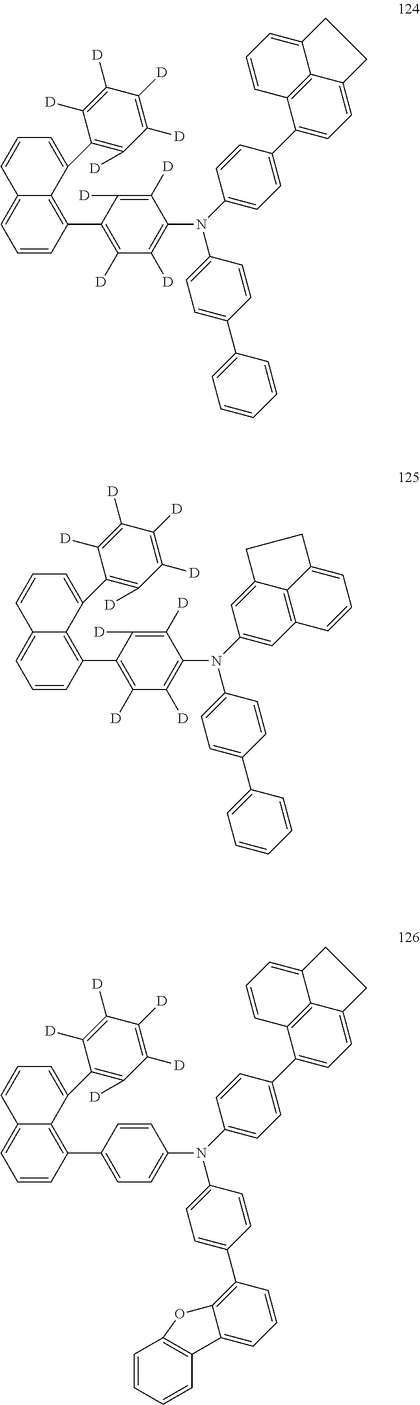 [SMILES: [2H]c1c([2H])c([2H])c(-c2cccc3cccc(-c4c([2H])c([2H])c(N(c5ccc(-c6ccccc6)cc5)c5ccc(-c6ccc7c8c(cccc68)CC7)cc5)c([2H])c4[2H])c23)c([2H])c1[2H] ; [2H]c1c([2H])c([2H])c(-c2cccc3cccc(-c4c([2H])c([2H])c(N(c5ccc(-c6ccccc6)cc5)c5cc6c7c(cccc7c5)CC6)c([2H])c4[2H])c23)c([2H])c1[2H] ; [2H]c1c([2H])c([2H])c(-c2cccc3cccc(-c4ccc(N(c5ccc(-c6ccc7c8c(cccc68)CC7)cc5)c5ccc(-c6cccc7c6oc6ccccc67)cc5)cc4)c23)c([2H])c1[2H]]

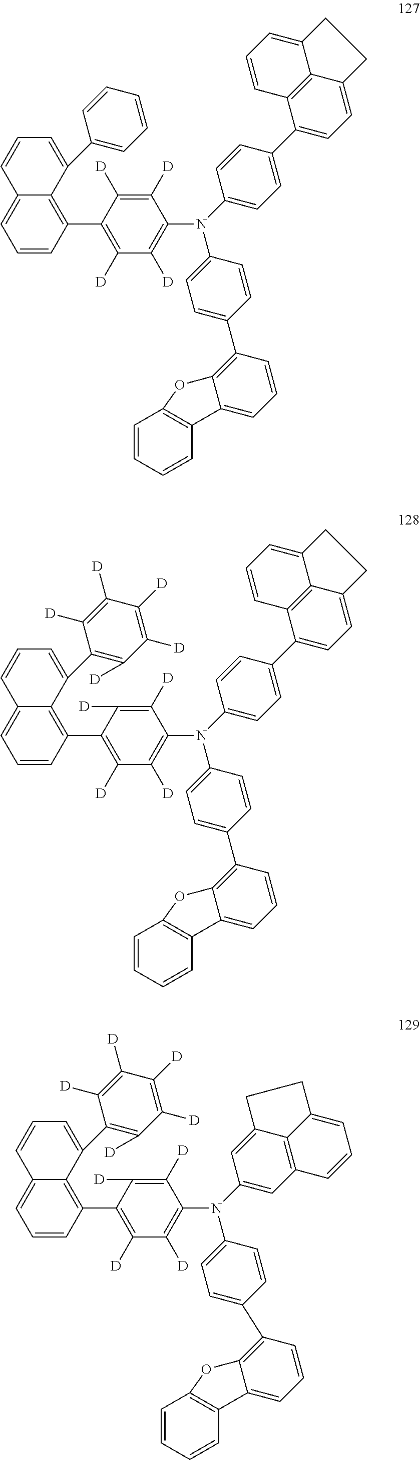 [SMILES: [2H]c1c([2H])c(N(c2ccc(-c3ccc4c5c(cccc35)CC4)cc2)c2ccc(-c3cccc4c3oc3ccccc34)cc2)c([2H])c([2H])c1-c1cccc2cccc(-c3ccccc3)c12 ; [2H]c1c([2H])c([2H])c(-c2cccc3cccc(-c4c([2H])c([2H])c(N(c5ccc(-c6ccc7c8c(cccc68)CC7)cc5)c5ccc(-c6cccc7c6oc6ccccc67)cc5)c([2H])c4[2H])c23)c([2H])c1[2H] ; [2H]c1c([2H])c([2H])c(-c2cccc3cccc(-c4c([2H])c([2H])c(N(c5ccc(-c6cccc7c6oc6ccccc67)cc5)c5cc6c7c(cccc7c5)CC6)c([2H])c4[2H])c23)c([2H])c1[2H]]

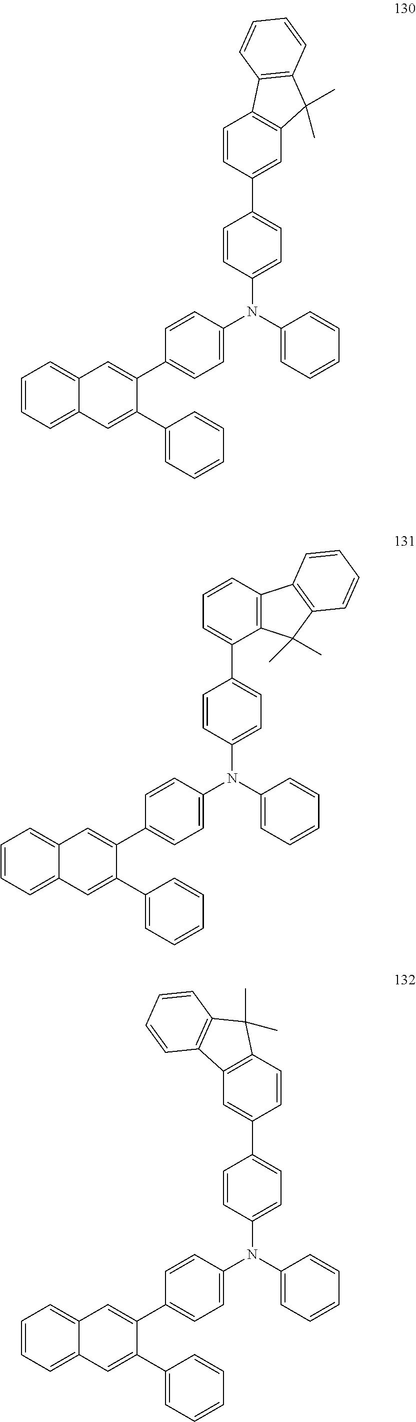 [SMILES: CC1(C)c2ccccc2-c2ccc(-c3ccc(N(c4ccccc4)c4ccc(-c5cc6ccccc6cc5-c5ccccc5)cc4)cc3)cc21 ; CC1(C)c2ccccc2-c2cccc(-c3ccc(N(c4ccccc4)c4ccc(-c5cc6ccccc6cc5-c5ccccc5)cc4)cc3)c21 ; CC1(C)c2ccccc2-c2cc(-c3ccc(N(c4ccccc4)c4ccc(-c5cc6ccccc6cc5-c5ccccc5)cc4)cc3)ccc21]

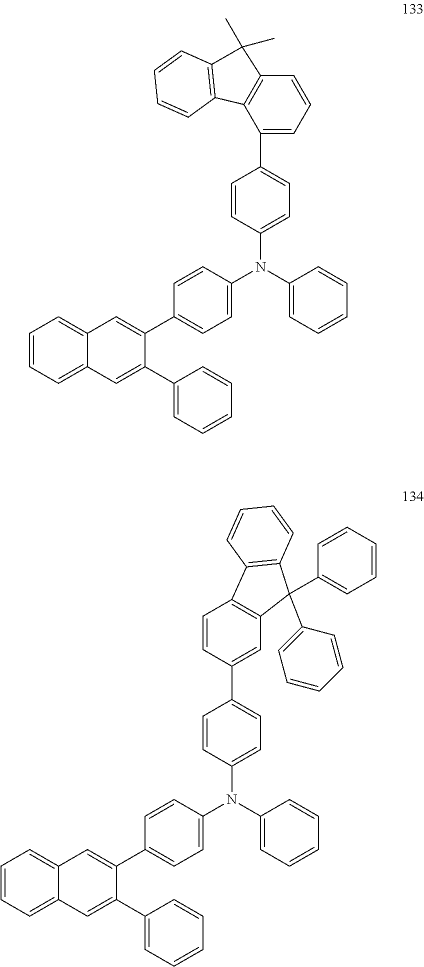 [SMILES: CC1(C)c2ccccc2-c2c(-c3ccc(N(c4ccccc4)c4ccc(-c5cc6ccccc6cc5-c5ccccc5)cc4)cc3)cccc21 ; c1ccc(-c2cc3ccccc3cc2-c2ccc(N(c3ccccc3)c3ccc(-c4ccc5c(c4)C(c4ccccc4)(c4ccccc4)c4ccccc4-5)cc3)cc2)cc1]

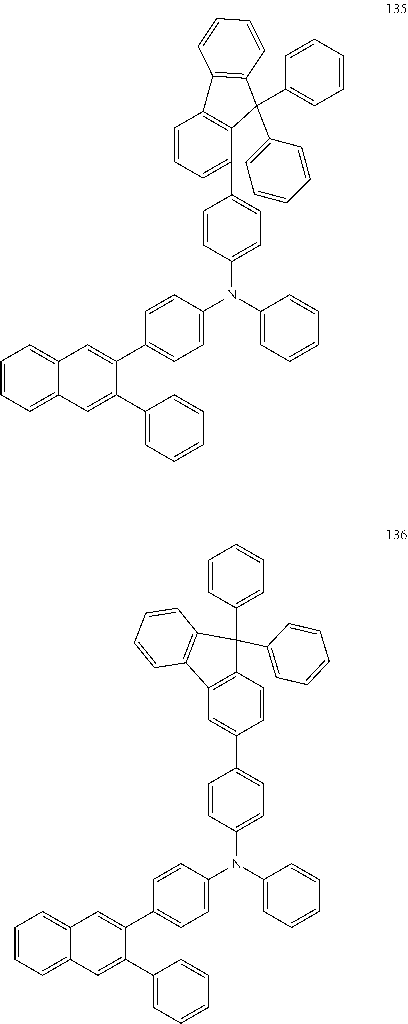 [SMILES: c1ccc(-c2cc3ccccc3cc2-c2ccc(N(c3ccccc3)c3ccc(-c4cccc5c4C(c4ccccc4)(c4ccccc4)c4ccccc4-5)cc3)cc2)cc1 ; c1ccc(-c2cc3ccccc3cc2-c2ccc(N(c3ccccc3)c3ccc(-c4ccc5c(c4)-c4ccccc4C5(c4ccccc4)c4ccccc4)cc3)cc2)cc1]

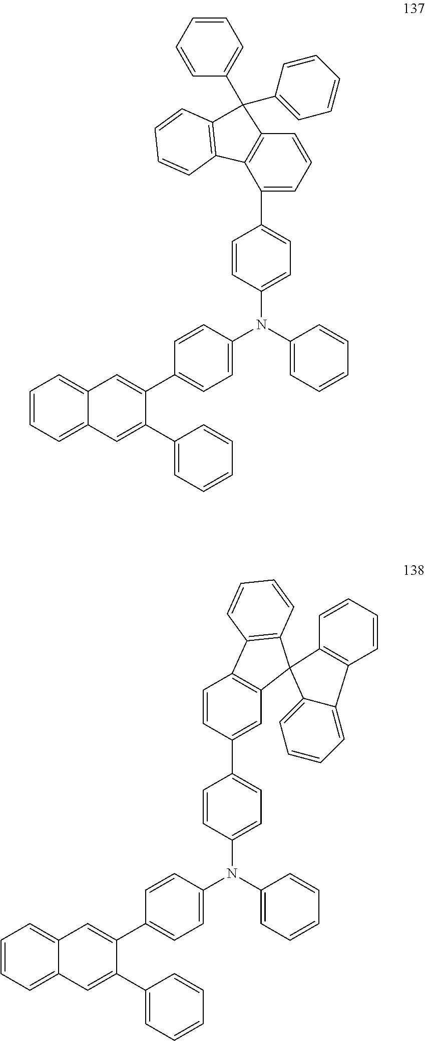 [SMILES: c1ccc(-c2cc3ccccc3cc2-c2ccc(N(c3ccccc3)c3ccc(-c4cccc5c4-c4ccccc4C5(c4ccccc4)c4ccccc4)cc3)cc2)cc1 ; c1ccc(-c2cc3ccccc3cc2-c2ccc(N(c3ccccc3)c3ccc(-c4ccc5c(c4)C4(c6ccccc6-c6ccccc64)c4ccccc4-5)cc3)cc2)cc1]

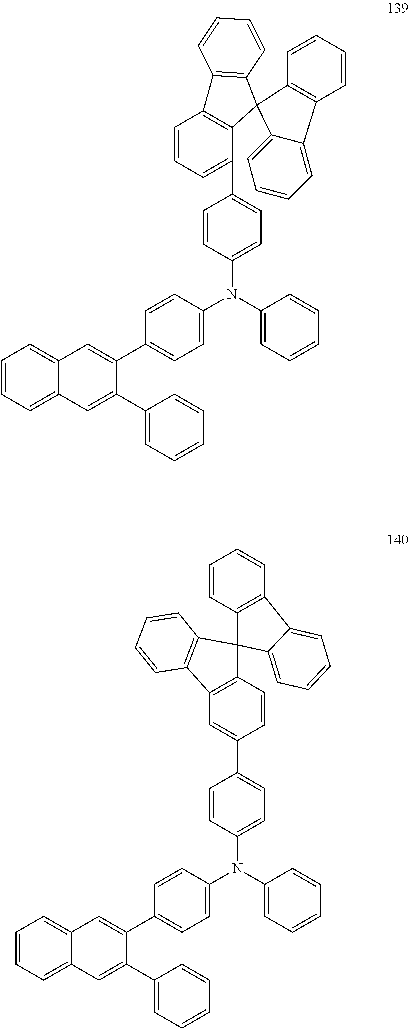 [SMILES: c1ccc(-c2cc3ccccc3cc2-c2ccc(N(c3ccccc3)c3ccc(-c4cccc5c4C4(c6ccccc6-c6ccccc64)c4ccccc4-5)cc3)cc2)cc1 ; c1ccc(-c2cc3ccccc3cc2-c2ccc(N(c3ccccc3)c3ccc(-c4ccc5c(c4)-c4ccccc4C54c5ccccc5-c5ccccc54)cc3)cc2)cc1]

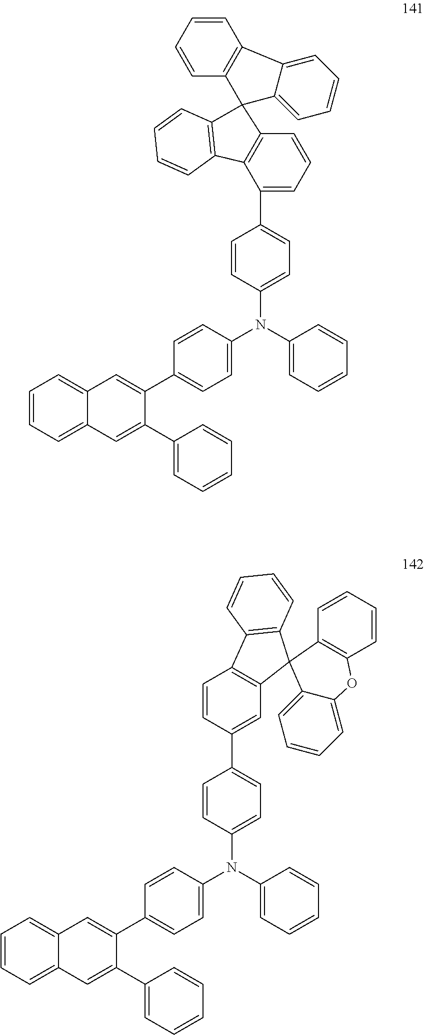 [SMILES: c1ccc(-c2cc3ccccc3cc2-c2ccc(N(c3ccccc3)c3ccc(-c4cccc5c4-c4ccccc4C54c5ccccc5-c5ccccc54)cc3)cc2)cc1 ; c1ccc(-c2cc3ccccc3cc2-c2ccc(N(c3ccccc3)c3ccc(-c4ccc5c(c4)C4(c6ccccc6Oc6ccccc64)c4ccccc4-5)cc3)cc2)cc1]

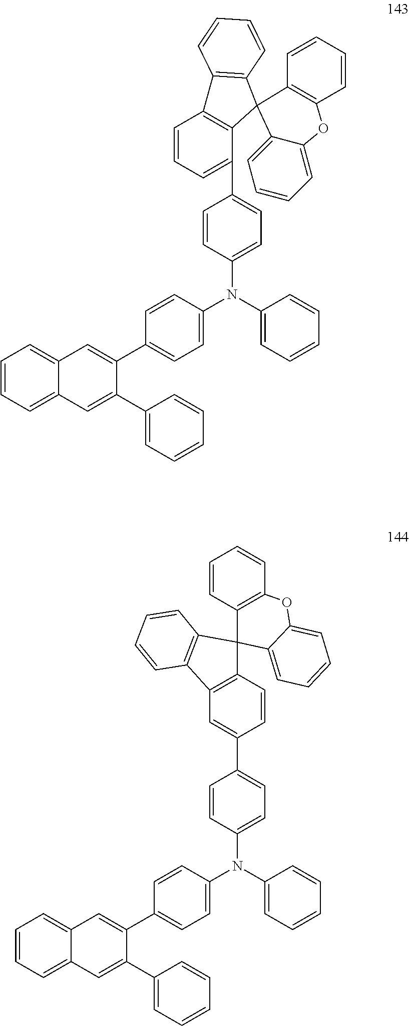 [SMILES: c1ccc(-c2cc3ccccc3cc2-c2ccc(N(c3ccccc3)c3ccc(-c4cccc5c4C4(c6ccccc6Oc6ccccc64)c4ccccc4-5)cc3)cc2)cc1 ; c1ccc(-c2cc3ccccc3cc2-c2ccc(N(c3ccccc3)c3ccc(-c4ccc5c(c4)-c4ccccc4C54c5ccccc5Oc5ccccc54)cc3)cc2)cc1]

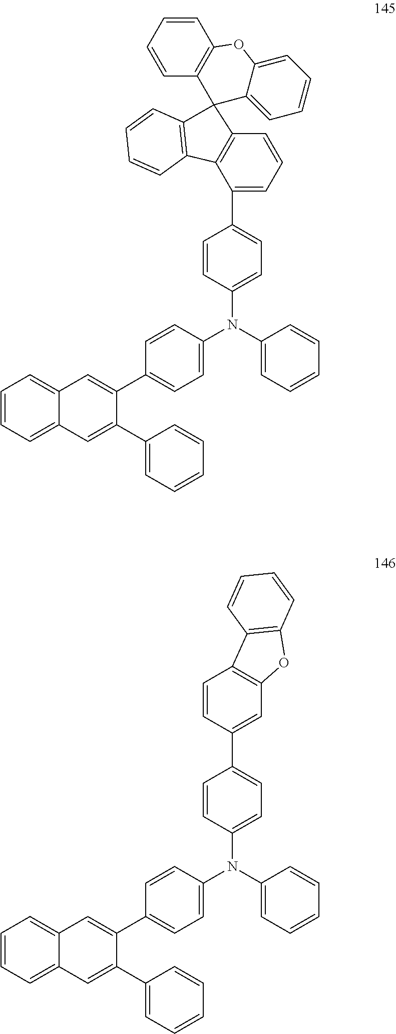 [SMILES: c1ccc(-c2cc3ccccc3cc2-c2ccc(N(c3ccccc3)c3ccc(-c4cccc5c4-c4ccccc4C54c5ccccc5Oc5ccccc54)cc3)cc2)cc1 ; c1ccc(-c2cc3ccccc3cc2-c2ccc(N(c3ccccc3)c3ccc(-c4ccc5c(c4)oc4ccccc45)cc3)cc2)cc1]

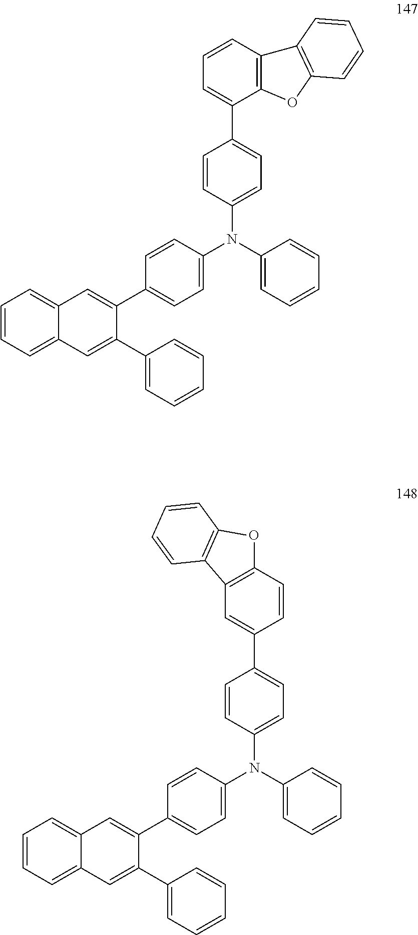 [SMILES: c1ccc(-c2cc3ccccc3cc2-c2ccc(N(c3ccccc3)c3ccc(-c4cccc5c4oc4ccccc45)cc3)cc2)cc1 ; c1ccc(-c2cc3ccccc3cc2-c2ccc(N(c3ccccc3)c3ccc(-c4ccc5oc6ccccc6c5c4)cc3)cc2)cc1]

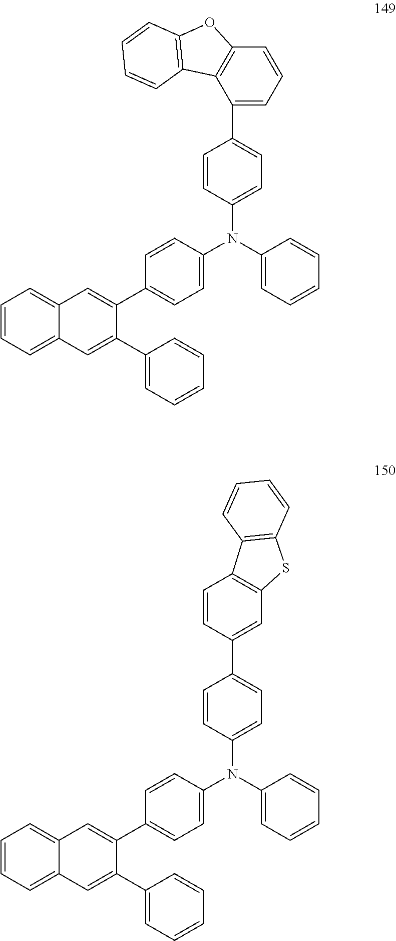 [SMILES: c1ccc(-c2cc3ccccc3cc2-c2ccc(N(c3ccccc3)c3ccc(-c4cccc5oc6ccccc6c45)cc3)cc2)cc1 ; c1ccc(-c2cc3ccccc3cc2-c2ccc(N(c3ccccc3)c3ccc(-c4ccc5c(c4)sc4ccccc45)cc3)cc2)cc1]

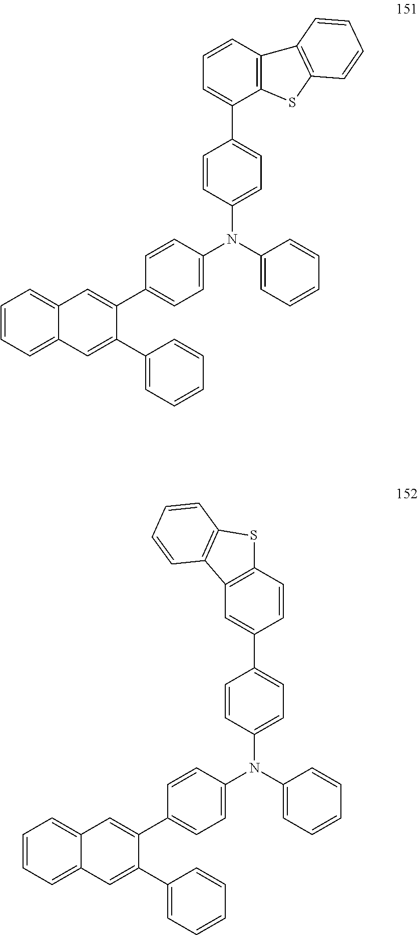 [SMILES: c1ccc(-c2cc3ccccc3cc2-c2ccc(N(c3ccccc3)c3ccc(-c4cccc5c4sc4ccccc45)cc3)cc2)cc1 ; c1ccc(-c2cc3ccccc3cc2-c2ccc(N(c3ccccc3)c3ccc(-c4ccc5sc6ccccc6c5c4)cc3)cc2)cc1]

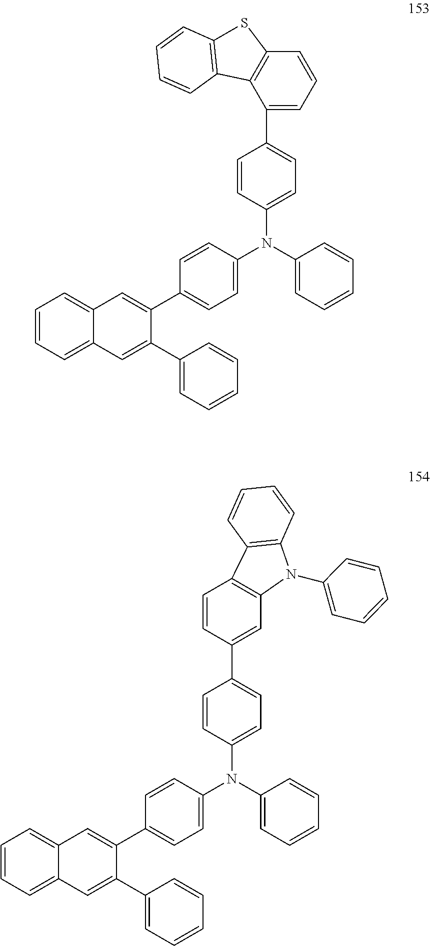 [SMILES: c1ccc(-c2cc3ccccc3cc2-c2ccc(N(c3ccccc3)c3ccc(-c4cccc5sc6ccccc6c45)cc3)cc2)cc1 ; c1ccc(-c2cc3ccccc3cc2-c2ccc(N(c3ccccc3)c3ccc(-c4ccc5c6ccccc6n(-c6ccccc6)c5c4)cc3)cc2)cc1]

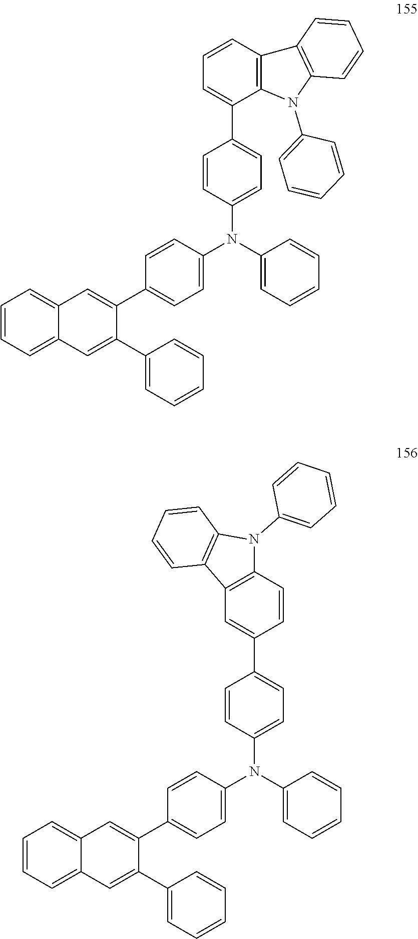 [SMILES: c1ccc(-c2cc3ccccc3cc2-c2ccc(N(c3ccccc3)c3ccc(-c4cccc5c6ccccc6n(-c6ccccc6)c45)cc3)cc2)cc1 ; c1ccc(-c2cc3ccccc3cc2-c2ccc(N(c3ccccc3)c3ccc(-c4ccc5c(c4)c4ccccc4n5-c4ccccc4)cc3)cc2)cc1]

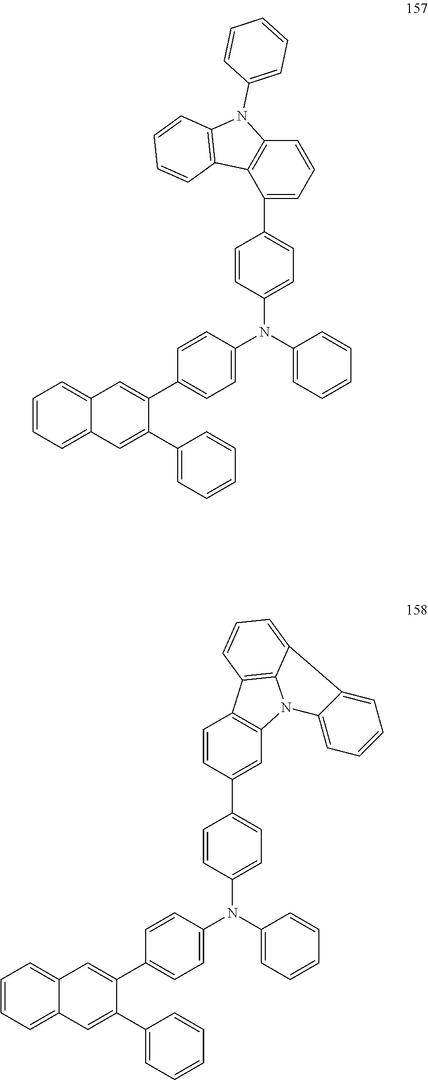 [SMILES: c1ccc(-c2cc3ccccc3cc2-c2ccc(N(c3ccccc3)c3ccc(-c4cccc5c4c4ccccc4n5-c4ccccc4)cc3)cc2)cc1 ; c1ccc(-c2cc3ccccc3cc2-c2ccc(N(c3ccccc3)c3ccc(-c4ccc5c6cccc7c8ccccc8n(c5c4)c76)cc3)cc2)cc1]

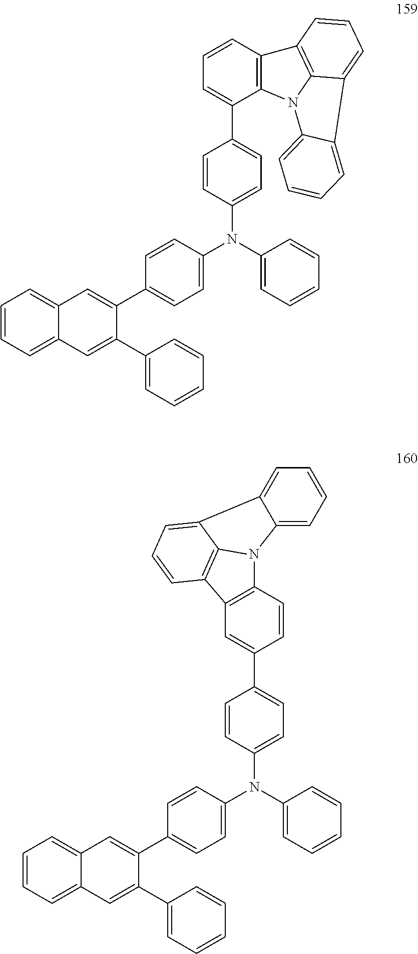 [SMILES: c1ccc(-c2cc3ccccc3cc2-c2ccc(N(c3ccccc3)c3ccc(-c4cccc5c6cccc7c8ccccc8n(c45)c76)cc3)cc2)cc1 ; c1ccc(-c2cc3ccccc3cc2-c2ccc(N(c3ccccc3)c3ccc(-c4ccc5c(c4)c4cccc6c7ccccc7n5c64)cc3)cc2)cc1]

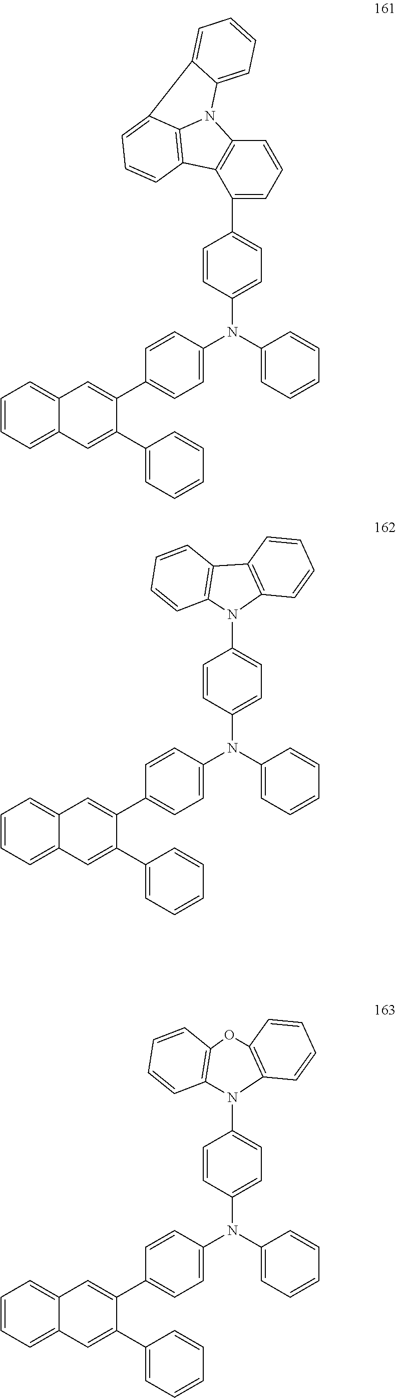 [SMILES: c1ccc(-c2cc3ccccc3cc2-c2ccc(N(c3ccccc3)c3ccc(-c4cccc5c4c4cccc6c7ccccc7n5c64)cc3)cc2)cc1 ; c1ccc(-c2cc3ccccc3cc2-c2ccc(N(c3ccccc3)c3ccc(-n4c5ccccc5c5ccccc54)cc3)cc2)cc1 ; c1ccc(-c2cc3ccccc3cc2-c2ccc(N(c3ccccc3)c3ccc(N4c5ccccc5Oc5ccccc54)cc3)cc2)cc1]

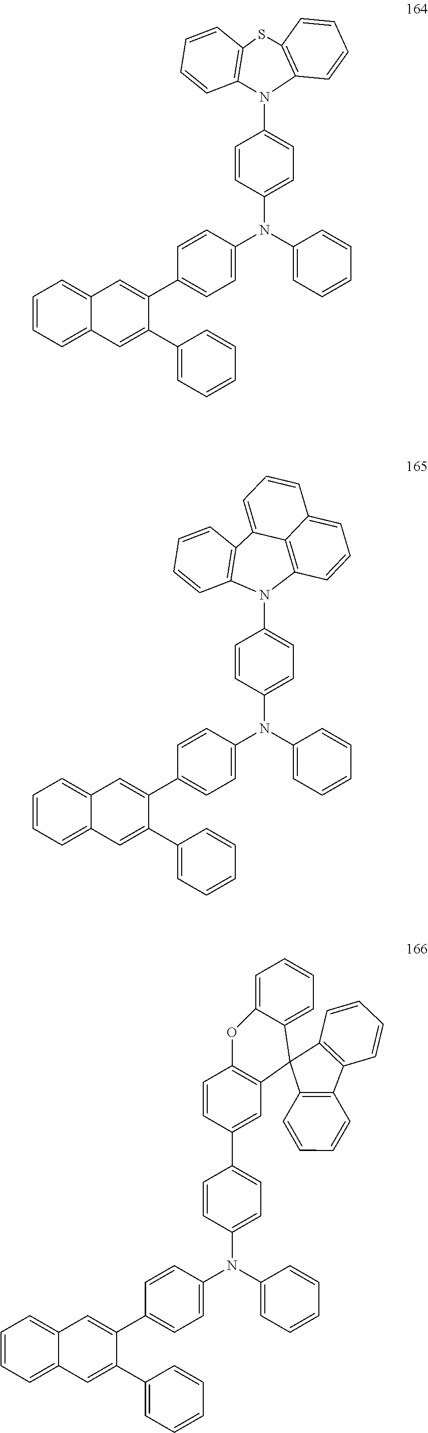 [SMILES: c1ccc(-c2cc3ccccc3cc2-c2ccc(N(c3ccccc3)c3ccc(N4c5ccccc5Sc5ccccc54)cc3)cc2)cc1 ; c1ccc(-c2cc3ccccc3cc2-c2ccc(N(c3ccccc3)c3ccc(N4c5ccccc5-c5cccc6cccc4c56)cc3)cc2)cc1 ; c1ccc(-c2cc3ccccc3cc2-c2ccc(N(c3ccccc3)c3ccc(-c4ccc5c(c4)C4(c6ccccc6O5)c5ccccc5-c5ccccc54)cc3)cc2)cc1]

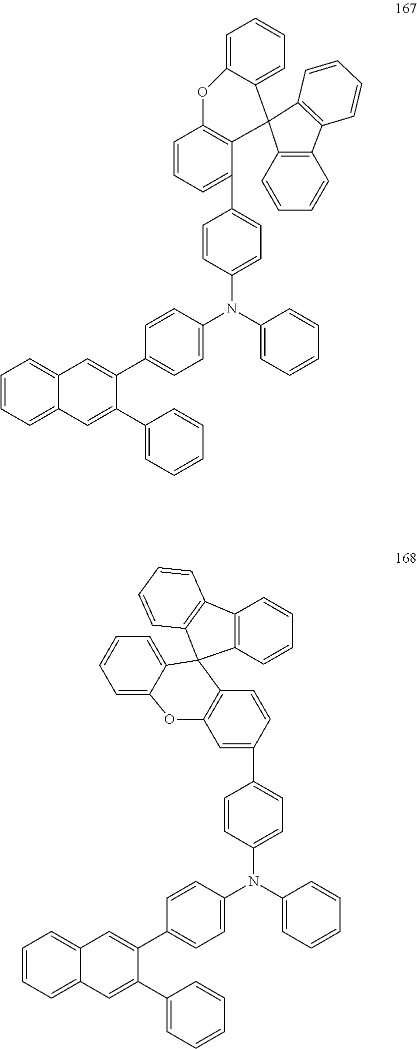 [SMILES: c1ccc(-c2cc3ccccc3cc2-c2ccc(N(c3ccccc3)c3ccc(-c4cccc5c4C4(c6ccccc6O5)c5ccccc5-c5ccccc54)cc3)cc2)cc1 ; c1ccc(-c2cc3ccccc3cc2-c2ccc(N(c3ccccc3)c3ccc(-c4ccc5c(c4)Oc4ccccc4C54c5ccccc5-c5ccccc54)cc3)cc2)cc1]

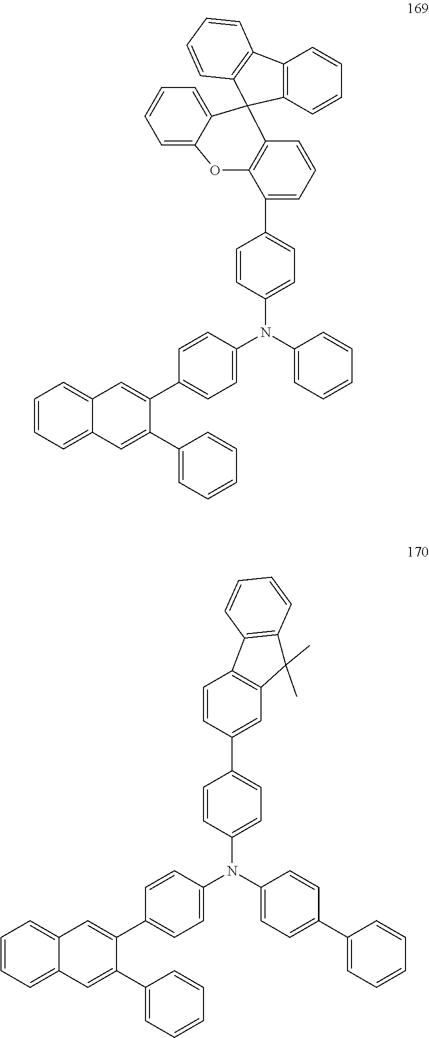 [SMILES: c1ccc(-c2cc3ccccc3cc2-c2ccc(N(c3ccccc3)c3ccc(-c4cccc5c4Oc4ccccc4C54c5ccccc5-c5ccccc54)cc3)cc2)cc1 ; CC1(C)c2ccccc2-c2ccc(-c3ccc(N(c4ccc(-c5ccccc5)cc4)c4ccc(-c5cc6ccccc6cc5-c5ccccc5)cc4)cc3)cc21]

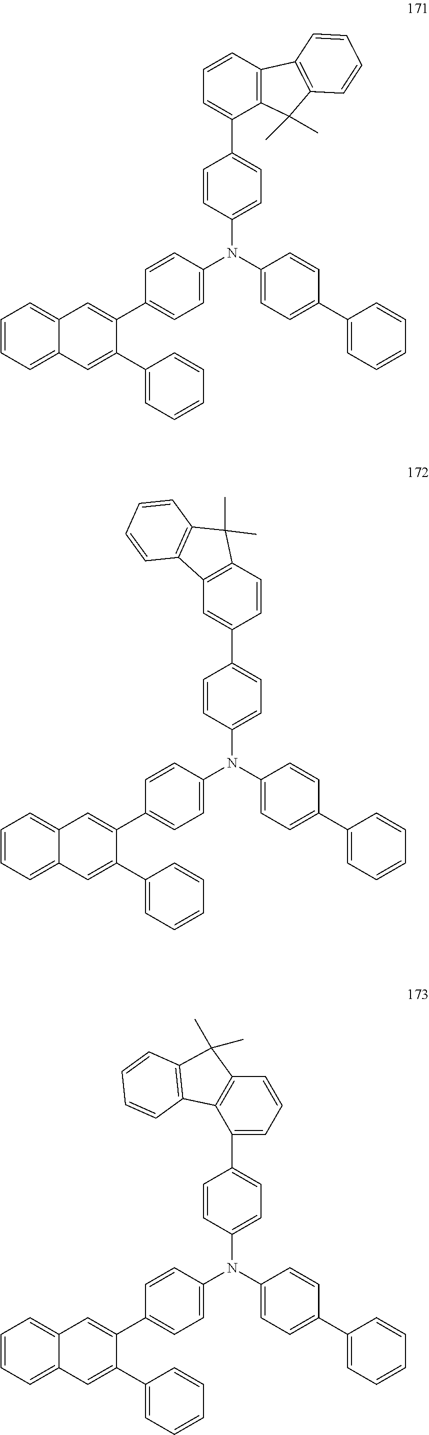 [SMILES: CC1(C)c2ccccc2-c2cccc(-c3ccc(N(c4ccc(-c5ccccc5)cc4)c4ccc(-c5cc6ccccc6cc5-c5ccccc5)cc4)cc3)c21 ; CC1(C)c2ccccc2-c2cc(-c3ccc(N(c4ccc(-c5ccccc5)cc4)c4ccc(-c5cc6ccccc6cc5-c5ccccc5)cc4)cc3)ccc21 ; CC1(C)c2ccccc2-c2c(-c3ccc(N(c4ccc(-c5ccccc5)cc4)c4ccc(-c5cc6ccccc6cc5-c5ccccc5)cc4)cc3)cccc21]

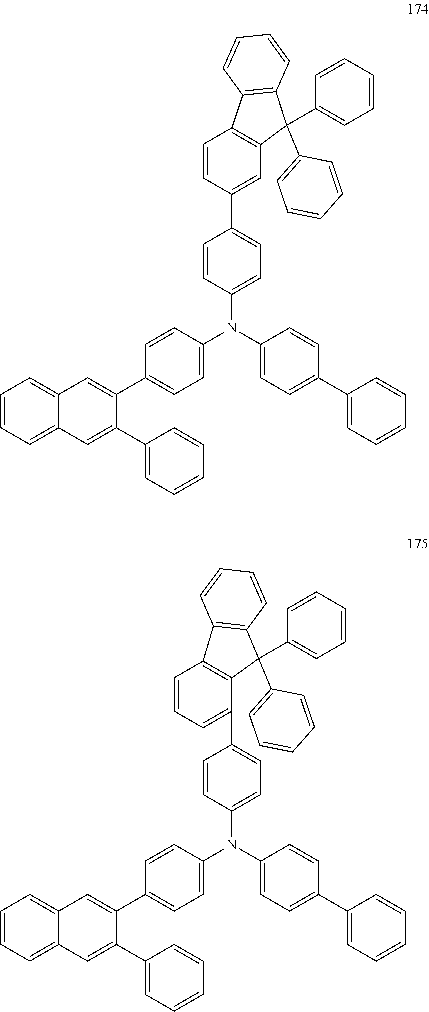 [SMILES: c1ccc(-c2ccc(N(c3ccc(-c4ccc5c(c4)C(c4ccccc4)(c4ccccc4)c4ccccc4-5)cc3)c3ccc(-c4cc5ccccc5cc4-c4ccccc4)cc3)cc2)cc1 ; c1ccc(-c2ccc(N(c3ccc(-c4cc5ccccc5cc4-c4ccccc4)cc3)c3ccc(-c4cccc5c4C(c4ccccc4)(c4ccccc4)c4ccccc4-5)cc3)cc2)cc1]

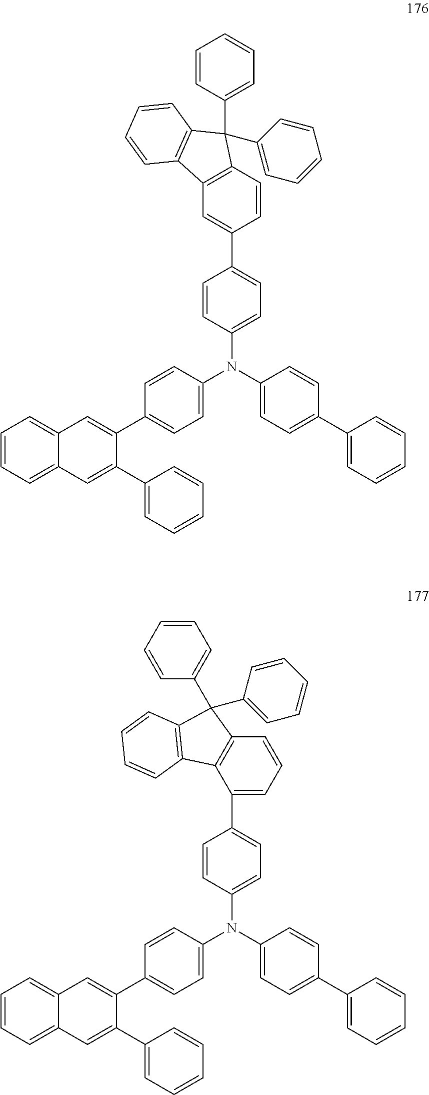 [SMILES: c1ccc(-c2ccc(N(c3ccc(-c4ccc5c(c4)-c4ccccc4C5(c4ccccc4)c4ccccc4)cc3)c3ccc(-c4cc5ccccc5cc4-c4ccccc4)cc3)cc2)cc1 ; c1ccc(-c2ccc(N(c3ccc(-c4cc5ccccc5cc4-c4ccccc4)cc3)c3ccc(-c4cccc5c4-c4ccccc4C5(c4ccccc4)c4ccccc4)cc3)cc2)cc1]

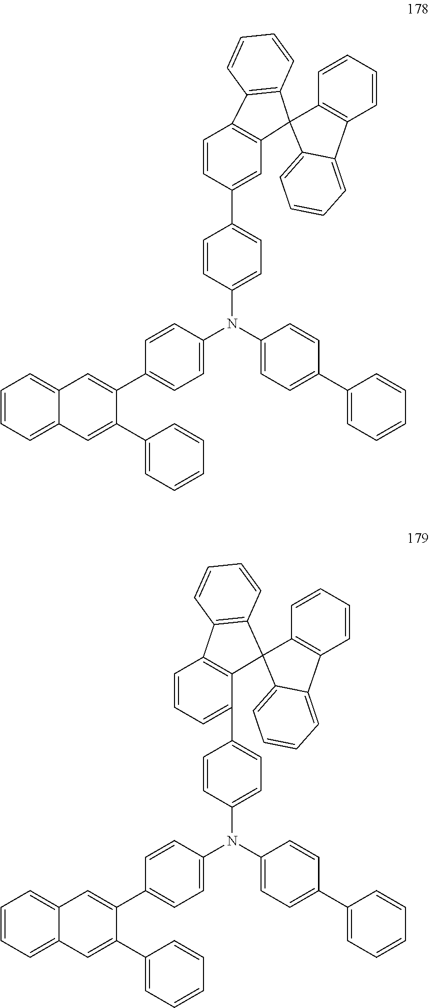 [SMILES: c1ccc(-c2ccc(N(c3ccc(-c4ccc5c(c4)C4(c6ccccc6-c6ccccc64)c4ccccc4-5)cc3)c3ccc(-c4cc5ccccc5cc4-c4ccccc4)cc3)cc2)cc1 ; c1ccc(-c2ccc(N(c3ccc(-c4cc5ccccc5cc4-c4ccccc4)cc3)c3ccc(-c4cccc5c4C4(c6ccccc6-c6ccccc64)c4ccccc4-5)cc3)cc2)cc1]

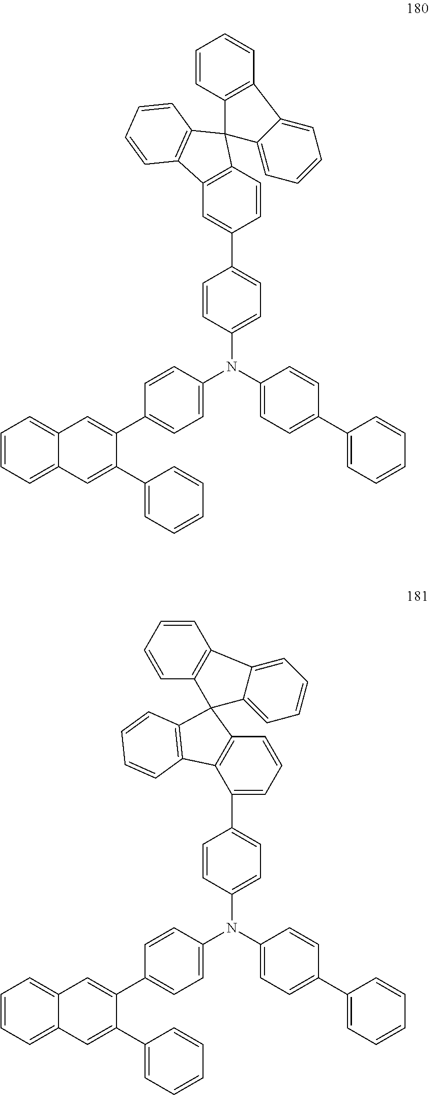 [SMILES: c1ccc(-c2ccc(N(c3ccc(-c4ccc5c(c4)-c4ccccc4C54c5ccccc5-c5ccccc54)cc3)c3ccc(-c4cc5ccccc5cc4-c4ccccc4)cc3)cc2)cc1 ; c1ccc(-c2ccc(N(c3ccc(-c4cc5ccccc5cc4-c4ccccc4)cc3)c3ccc(-c4cccc5c4-c4ccccc4C54c5ccccc5-c5ccccc54)cc3)cc2)cc1]

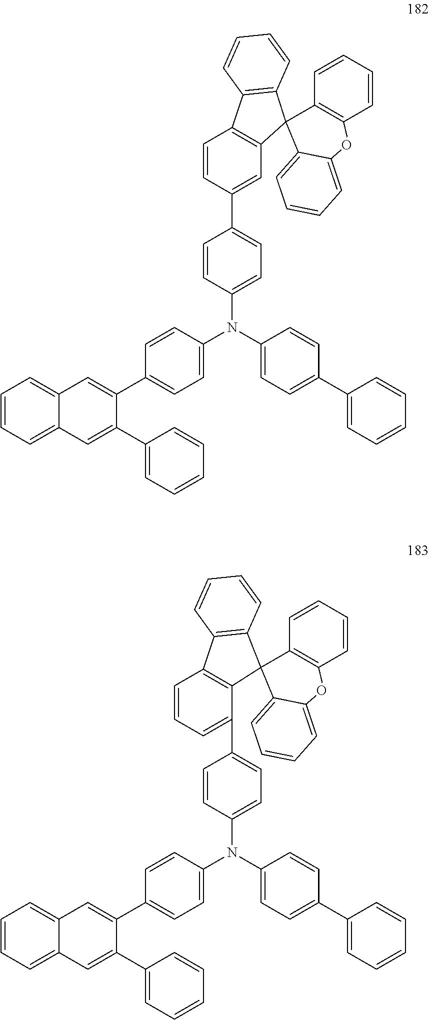 [SMILES: c1ccc(-c2ccc(N(c3ccc(-c4ccc5c(c4)C4(c6ccccc6Oc6ccccc64)c4ccccc4-5)cc3)c3ccc(-c4cc5ccccc5cc4-c4ccccc4)cc3)cc2)cc1 ; c1ccc(-c2ccc(N(c3ccc(-c4cc5ccccc5cc4-c4ccccc4)cc3)c3ccc(-c4cccc5c4C4(c6ccccc6Oc6ccccc64)c4ccccc4-5)cc3)cc2)cc1]

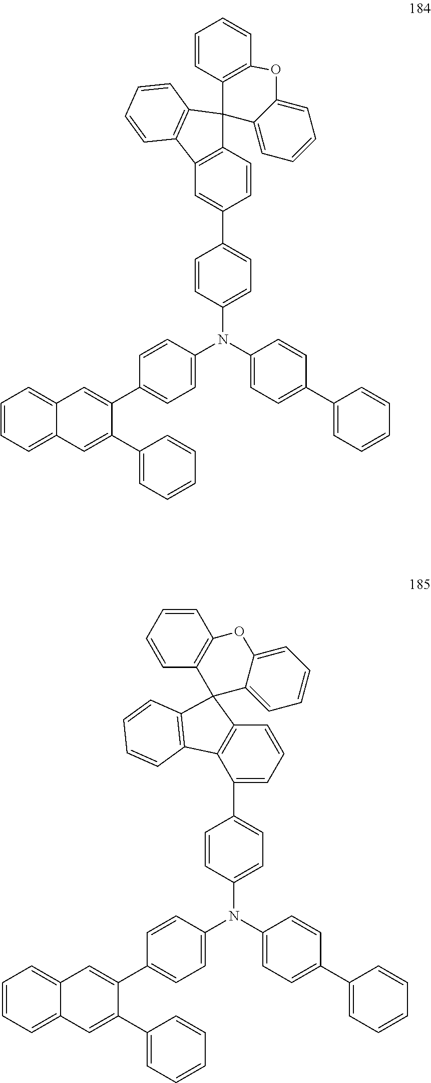 [SMILES: c1ccc(-c2ccc(N(c3ccc(-c4ccc5c(c4)-c4ccccc4C54c5ccccc5Oc5ccccc54)cc3)c3ccc(-c4cc5ccccc5cc4-c4ccccc4)cc3)cc2)cc1 ; c1ccc(-c2ccc(N(c3ccc(-c4cc5ccccc5cc4-c4ccccc4)cc3)c3ccc(-c4cccc5c4-c4ccccc4C54c5ccccc5Oc5ccccc54)cc3)cc2)cc1]

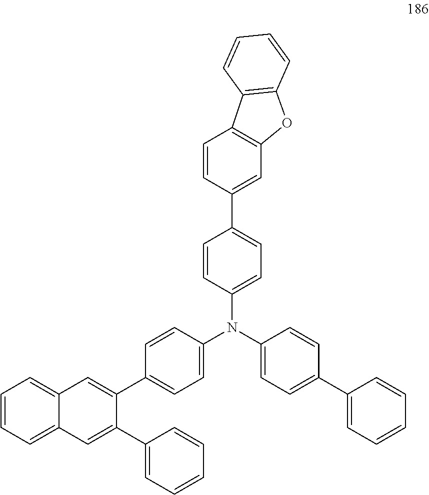 [SMILES: c1ccc(-c2ccc(N(c3ccc(-c4ccc5c(c4)oc4ccccc45)cc3)c3ccc(-c4cc5ccccc5cc4-c4ccccc4)cc3)cc2)cc1]

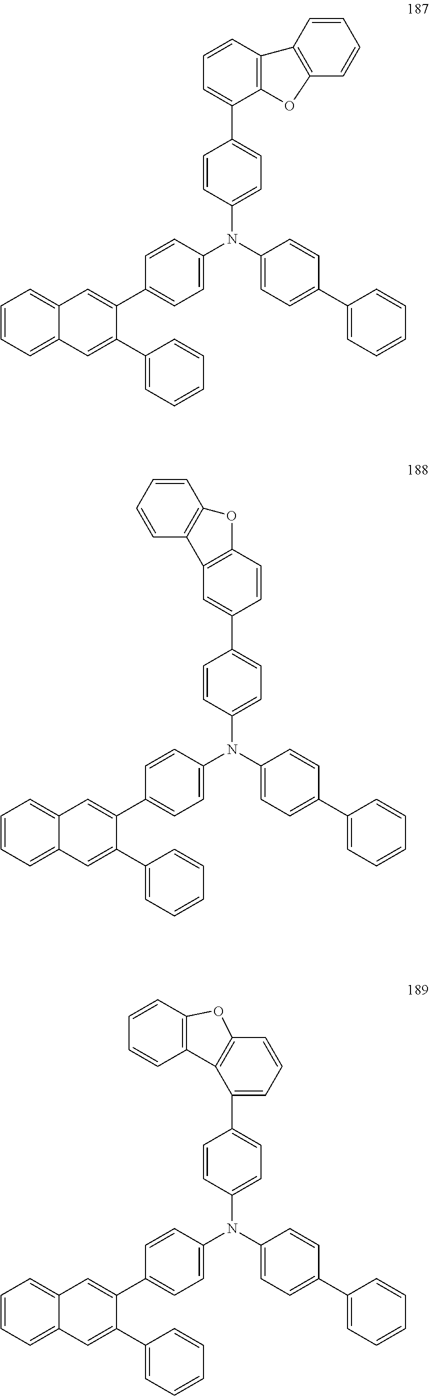 [SMILES: c1ccc(-c2ccc(N(c3ccc(-c4cc5ccccc5cc4-c4ccccc4)cc3)c3ccc(-c4cccc5c4oc4ccccc45)cc3)cc2)cc1 ; c1ccc(-c2ccc(N(c3ccc(-c4ccc5oc6ccccc6c5c4)cc3)c3ccc(-c4cc5ccccc5cc4-c4ccccc4)cc3)cc2)cc1 ; c1ccc(-c2ccc(N(c3ccc(-c4cc5ccccc5cc4-c4ccccc4)cc3)c3ccc(-c4cccc5oc6ccccc6c45)cc3)cc2)cc1]

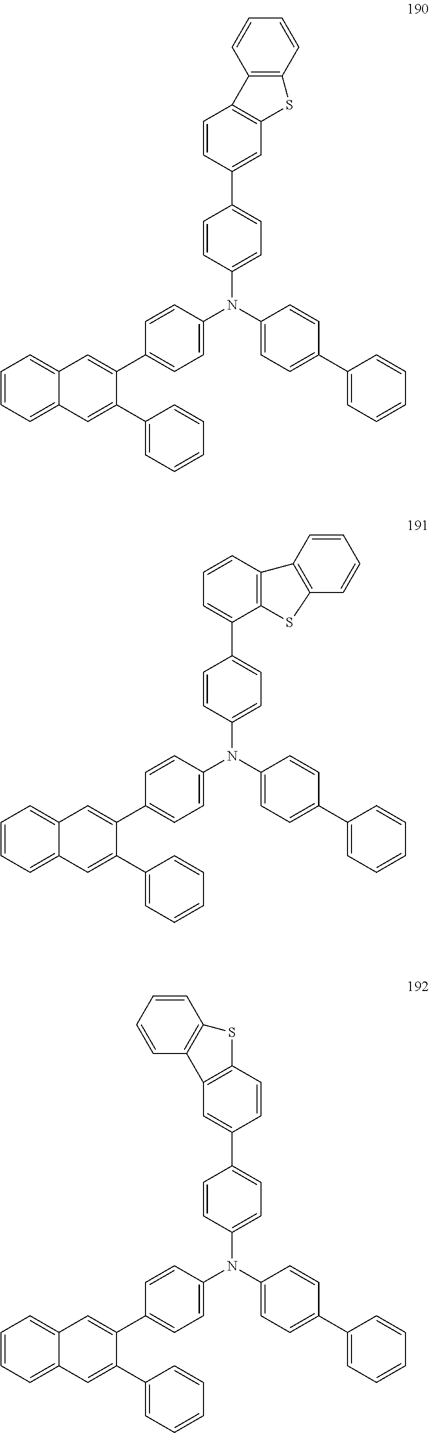 [SMILES: c1ccc(-c2ccc(N(c3ccc(-c4ccc5c(c4)sc4ccccc45)cc3)c3ccc(-c4cc5ccccc5cc4-c4ccccc4)cc3)cc2)cc1 ; c1ccc(-c2ccc(N(c3ccc(-c4cc5ccccc5cc4-c4ccccc4)cc3)c3ccc(-c4cccc5c4sc4ccccc45)cc3)cc2)cc1 ; c1ccc(-c2ccc(N(c3ccc(-c4ccc5sc6ccccc6c5c4)cc3)c3ccc(-c4cc5ccccc5cc4-c4ccccc4)cc3)cc2)cc1]

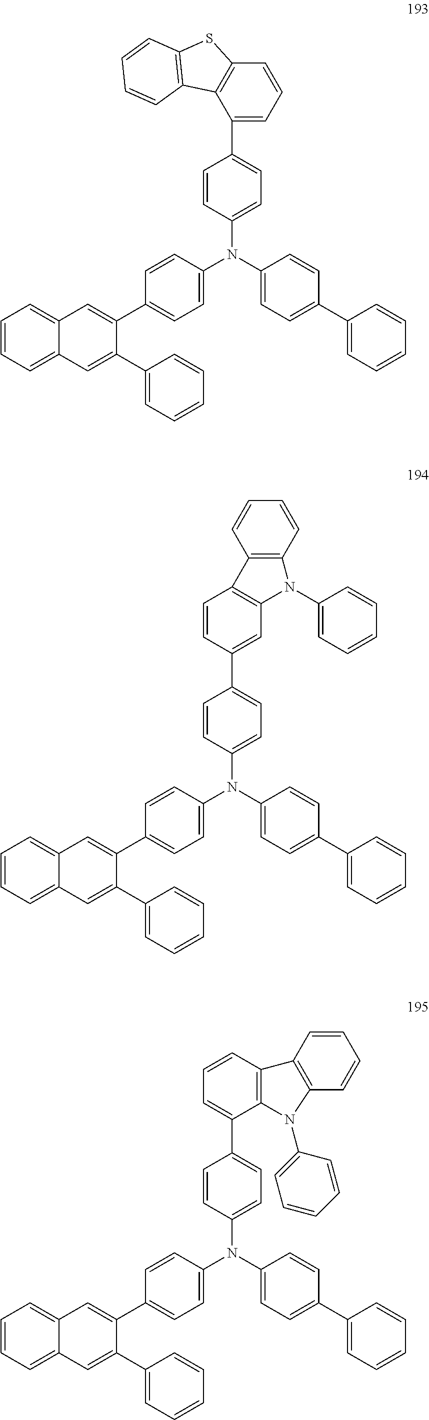 [SMILES: c1ccc(-c2ccc(N(c3ccc(-c4cc5ccccc5cc4-c4ccccc4)cc3)c3ccc(-c4cccc5sc6ccccc6c45)cc3)cc2)cc1 ; c1ccc(-c2ccc(N(c3ccc(-c4ccc5c6ccccc6n(-c6ccccc6)c5c4)cc3)c3ccc(-c4cc5ccccc5cc4-c4ccccc4)cc3)cc2)cc1 ; c1ccc(-c2ccc(N(c3ccc(-c4cc5ccccc5cc4-c4ccccc4)cc3)c3ccc(-c4cccc5c6ccccc6n(-c6ccccc6)c45)cc3)cc2)cc1]

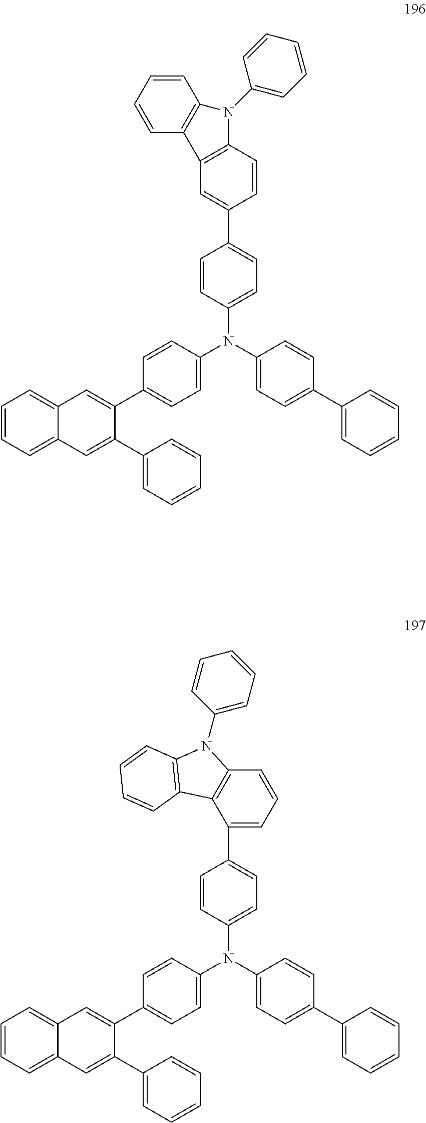 [SMILES: c1ccc(-c2ccc(N(c3ccc(-c4ccc5c(c4)c4ccccc4n5-c4ccccc4)cc3)c3ccc(-c4cc5ccccc5cc4-c4ccccc4)cc3)cc2)cc1 ; c1ccc(-c2ccc(N(c3ccc(-c4cc5ccccc5cc4-c4ccccc4)cc3)c3ccc(-c4cccc5c4c4ccccc4n5-c4ccccc4)cc3)cc2)cc1]

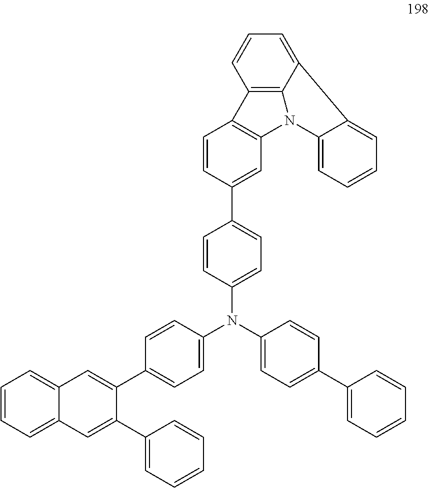 [SMILES: c1ccc(-c2ccc(N(c3ccc(-c4ccc5c6cccc7c8ccccc8n(c5c4)c76)cc3)c3ccc(-c4cc5ccccc5cc4-c4ccccc4)cc3)cc2)cc1]

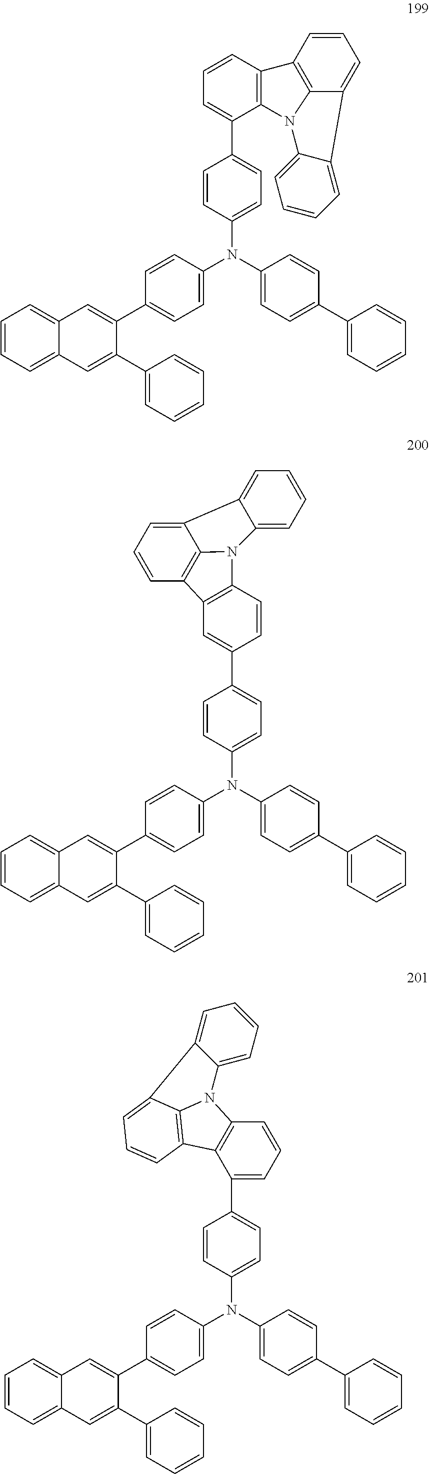 [SMILES: c1ccc(-c2ccc(N(c3ccc(-c4cc5ccccc5cc4-c4ccccc4)cc3)c3ccc(-c4cccc5c6cccc7c8ccccc8n(c45)c76)cc3)cc2)cc1 ; c1ccc(-c2ccc(N(c3ccc(-c4ccc5c(c4)c4cccc6c7ccccc7n5c64)cc3)c3ccc(-c4cc5ccccc5cc4-c4ccccc4)cc3)cc2)cc1 ; c1ccc(-c2ccc(N(c3ccc(-c4cc5ccccc5cc4-c4ccccc4)cc3)c3ccc(-c4cccc5c4c4cccc6c7ccccc7n5c64)cc3)cc2)cc1]

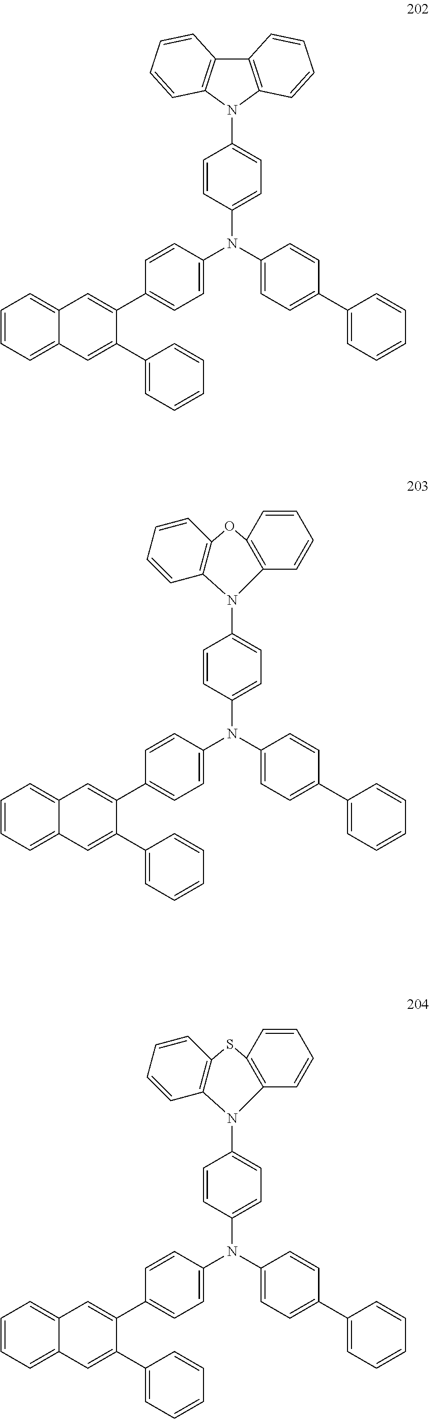 [SMILES: c1ccc(-c2ccc(N(c3ccc(-c4cc5ccccc5cc4-c4ccccc4)cc3)c3ccc(-n4c5ccccc5c5ccccc54)cc3)cc2)cc1 ; c1ccc(-c2ccc(N(c3ccc(-c4cc5ccccc5cc4-c4ccccc4)cc3)c3ccc(N4c5ccccc5Oc5ccccc54)cc3)cc2)cc1 ; c1ccc(-c2ccc(N(c3ccc(-c4cc5ccccc5cc4-c4ccccc4)cc3)c3ccc(N4c5ccccc5Sc5ccccc54)cc3)cc2)cc1]

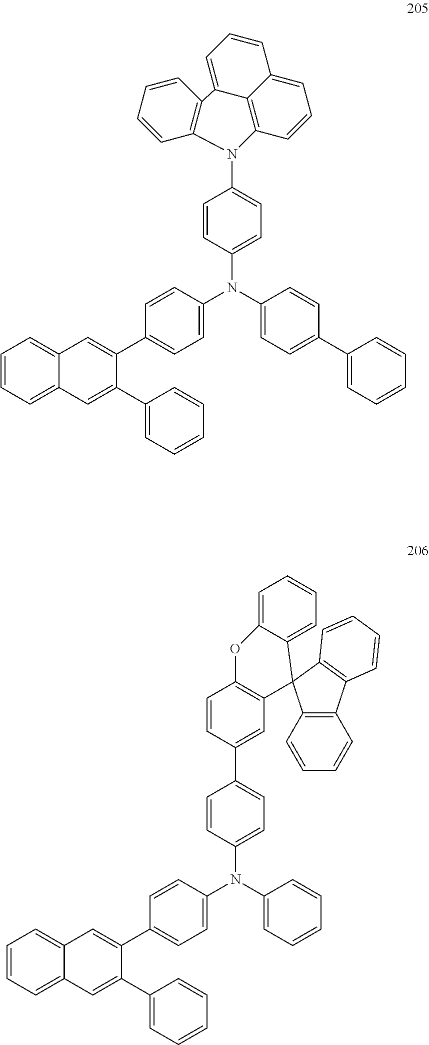 [SMILES: c1ccc(-c2ccc(N(c3ccc(-c4cc5ccccc5cc4-c4ccccc4)cc3)c3ccc(N4c5ccccc5-c5cccc6cccc4c56)cc3)cc2)cc1 ; c1ccc(-c2cc3ccccc3cc2-c2ccc(N(c3ccccc3)c3ccc(-c4ccc5c(c4)C4(c6ccccc6O5)c5ccccc5-c5ccccc54)cc3)cc2)cc1]

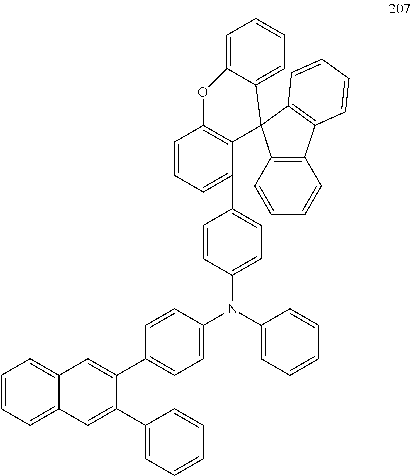 [SMILES: c1ccc(-c2cc3ccccc3cc2-c2ccc(N(c3ccccc3)c3ccc(-c4cccc5c4C4(c6ccccc6O5)c5ccccc5-c5ccccc54)cc3)cc2)cc1]

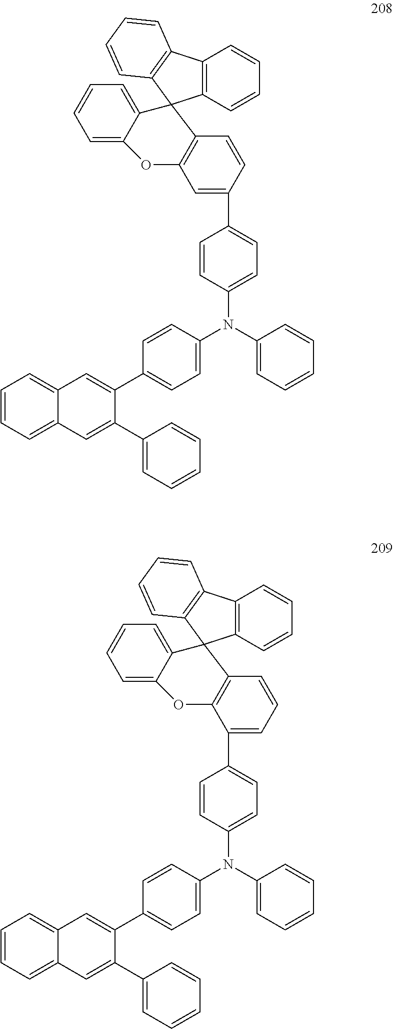 [SMILES: c1ccc(-c2cc3ccccc3cc2-c2ccc(N(c3ccccc3)c3ccc(-c4ccc5c(c4)Oc4ccccc4C54c5ccccc5-c5ccccc54)cc3)cc2)cc1 ; c1ccc(-c2cc3ccccc3cc2-c2ccc(N(c3ccccc3)c3ccc(-c4cccc5c4Oc4ccccc4C54c5ccccc5-c5ccccc54)cc3)cc2)cc1]

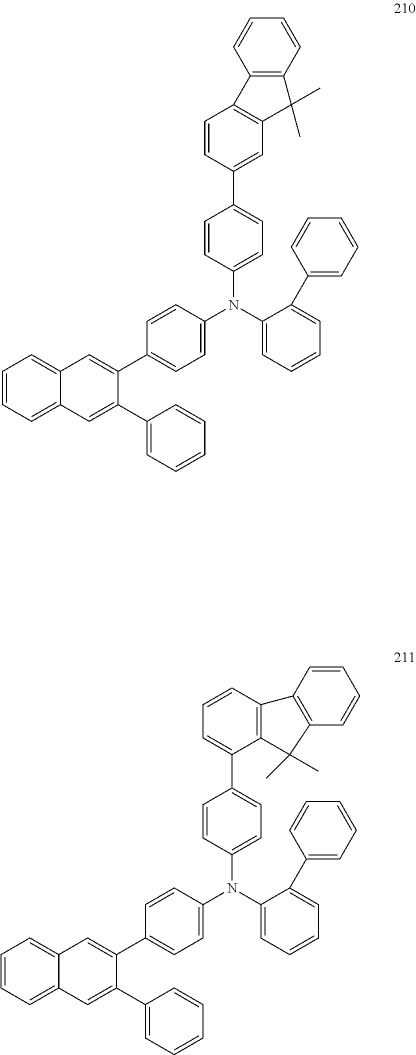 [SMILES: CC1(C)c2ccccc2-c2ccc(-c3ccc(N(c4ccc(-c5cc6ccccc6cc5-c5ccccc5)cc4)c4ccccc4-c4ccccc4)cc3)cc21 ; CC1(C)c2ccccc2-c2cccc(-c3ccc(N(c4ccc(-c5cc6ccccc6cc5-c5ccccc5)cc4)c4ccccc4-c4ccccc4)cc3)c21]

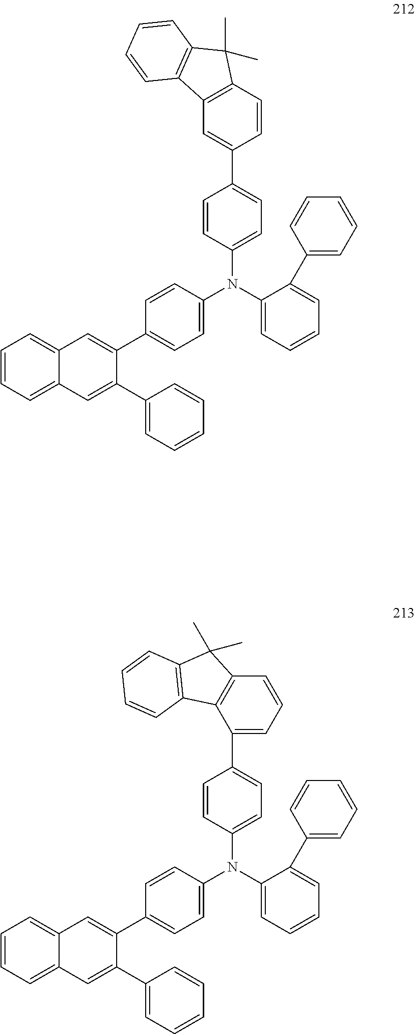 [SMILES: CC1(C)c2ccccc2-c2cc(-c3ccc(N(c4ccc(-c5cc6ccccc6cc5-c5ccccc5)cc4)c4ccccc4-c4ccccc4)cc3)ccc21 ; CC1(C)c2ccccc2-c2c(-c3ccc(N(c4ccc(-c5cc6ccccc6cc5-c5ccccc5)cc4)c4ccccc4-c4ccccc4)cc3)cccc21]

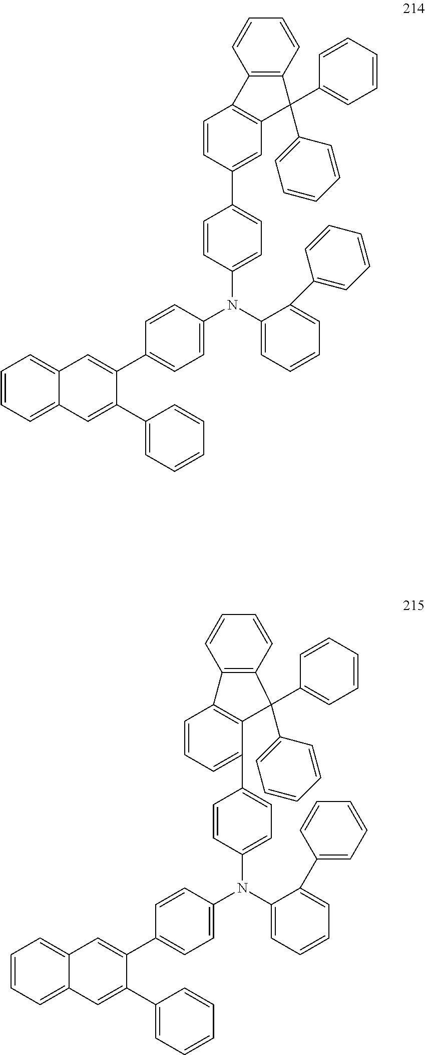 [SMILES: c1ccc(-c2cc3ccccc3cc2-c2ccc(N(c3ccc(-c4ccc5c(c4)C(c4ccccc4)(c4ccccc4)c4ccccc4-5)cc3)c3ccccc3-c3ccccc3)cc2)cc1 ; c1ccc(-c2cc3ccccc3cc2-c2ccc(N(c3ccc(-c4cccc5c4C(c4ccccc4)(c4ccccc4)c4ccccc4-5)cc3)c3ccccc3-c3ccccc3)cc2)cc1]

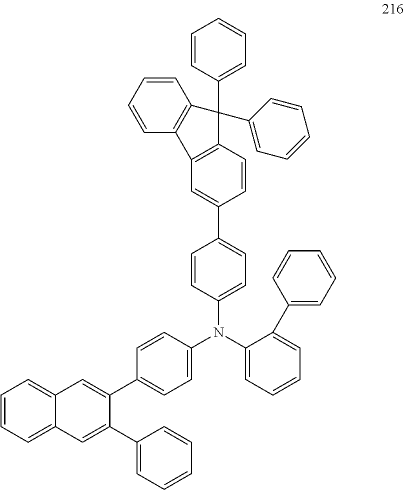 [SMILES: c1ccc(-c2cc3ccccc3cc2-c2ccc(N(c3ccc(-c4ccc5c(c4)-c4ccccc4C5(c4ccccc4)c4ccccc4)cc3)c3ccccc3-c3ccccc3)cc2)cc1]

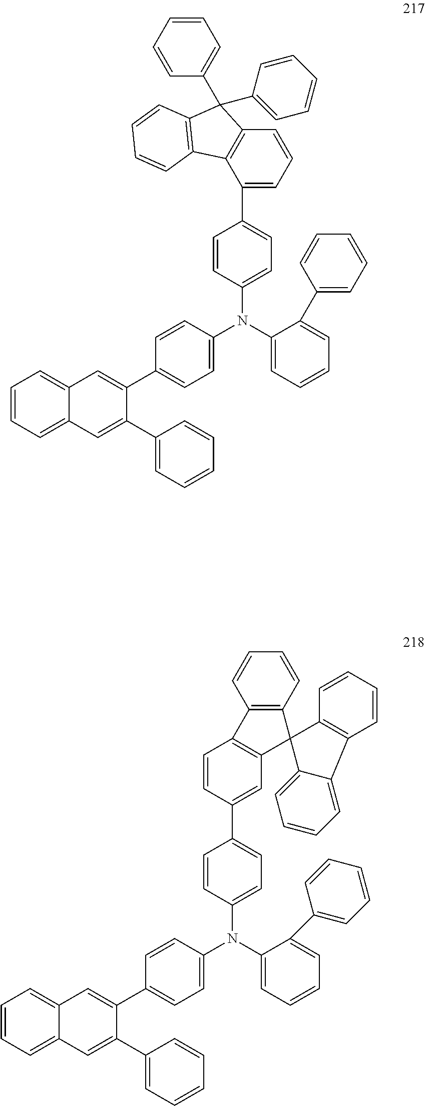 [SMILES: c1ccc(-c2cc3ccccc3cc2-c2ccc(N(c3ccc(-c4cccc5c4-c4ccccc4C5(c4ccccc4)c4ccccc4)cc3)c3ccccc3-c3ccccc3)cc2)cc1 ; c1ccc(-c2cc3ccccc3cc2-c2ccc(N(c3ccc(-c4ccc5c(c4)C4(c6ccccc6-c6ccccc64)c4ccccc4-5)cc3)c3ccccc3-c3ccccc3)cc2)cc1]

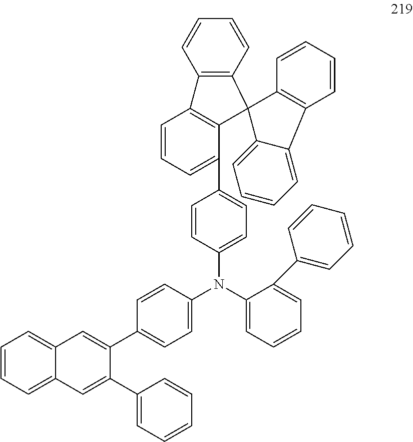 [SMILES: c1ccc(-c2cc3ccccc3cc2-c2ccc(N(c3ccc(-c4cccc5c4C4(c6ccccc6-c6ccccc64)c4ccccc4-5)cc3)c3ccccc3-c3ccccc3)cc2)cc1]

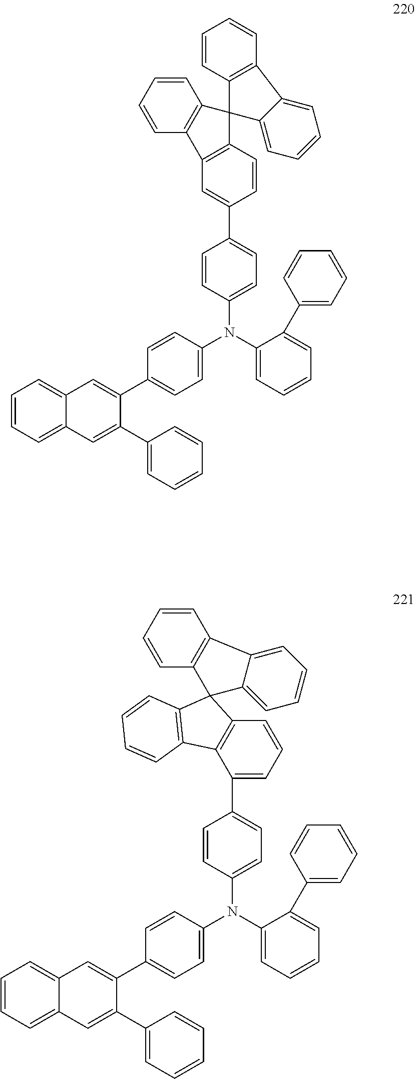 [SMILES: c1ccc(-c2cc3ccccc3cc2-c2ccc(N(c3ccc(-c4ccc5c(c4)-c4ccccc4C54c5ccccc5-c5ccccc54)cc3)c3ccccc3-c3ccccc3)cc2)cc1 ; c1ccc(-c2cc3ccccc3cc2-c2ccc(N(c3ccc(-c4cccc5c4-c4ccccc4C54c5ccccc5-c5ccccc54)cc3)c3ccccc3-c3ccccc3)cc2)cc1]

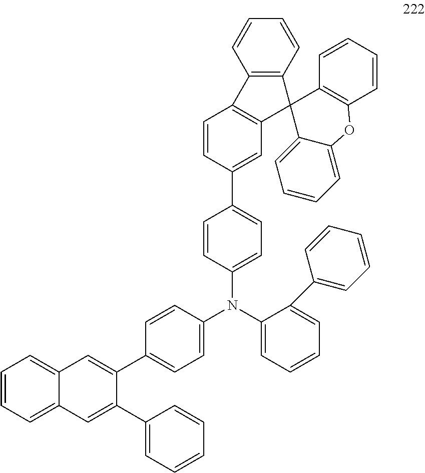 [SMILES: c1ccc(-c2cc3ccccc3cc2-c2ccc(N(c3ccc(-c4ccc5c(c4)C4(c6ccccc6Oc6ccccc64)c4ccccc4-5)cc3)c3ccccc3-c3ccccc3)cc2)cc1]

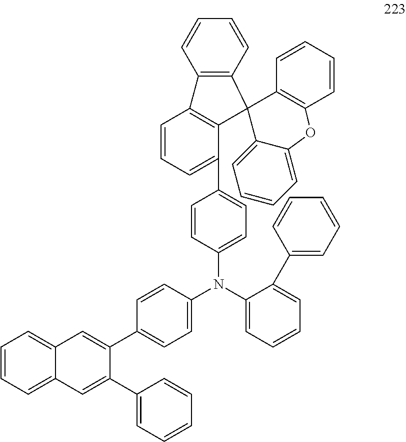 [SMILES: c1ccc(-c2cc3ccccc3cc2-c2ccc(N(c3ccc(-c4cccc5c4C4(c6ccccc6Oc6ccccc64)c4ccccc4-5)cc3)c3ccccc3-c3ccccc3)cc2)cc1]

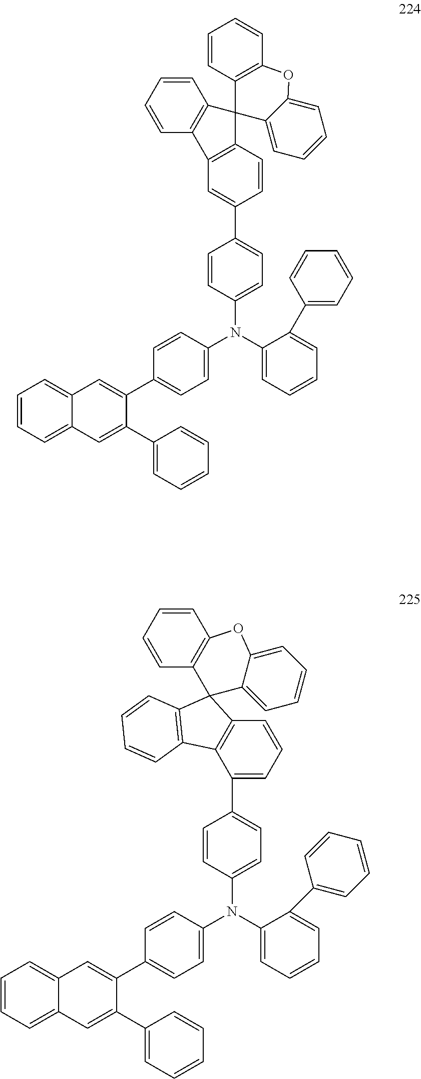 [SMILES: c1ccc(-c2cc3ccccc3cc2-c2ccc(N(c3ccc(-c4ccc5c(c4)-c4ccccc4C54c5ccccc5Oc5ccccc54)cc3)c3ccccc3-c3ccccc3)cc2)cc1 ; c1ccc(-c2cc3ccccc3cc2-c2ccc(N(c3ccc(-c4cccc5c4-c4ccccc4C54c5ccccc5Oc5ccccc54)cc3)c3ccccc3-c3ccccc3)cc2)cc1]

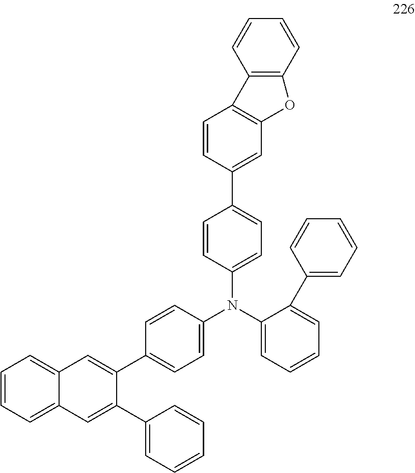 [SMILES: c1ccc(-c2cc3ccccc3cc2-c2ccc(N(c3ccc(-c4ccc5c(c4)oc4ccccc45)cc3)c3ccccc3-c3ccccc3)cc2)cc1]

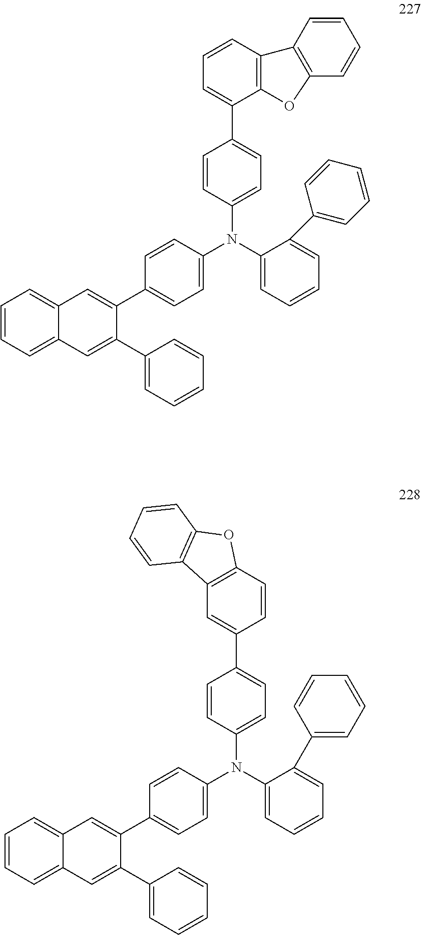 [SMILES: c1ccc(-c2cc3ccccc3cc2-c2ccc(N(c3ccc(-c4cccc5c4oc4ccccc45)cc3)c3ccccc3-c3ccccc3)cc2)cc1 ; c1ccc(-c2cc3ccccc3cc2-c2ccc(N(c3ccc(-c4ccc5oc6ccccc6c5c4)cc3)c3ccccc3-c3ccccc3)cc2)cc1]

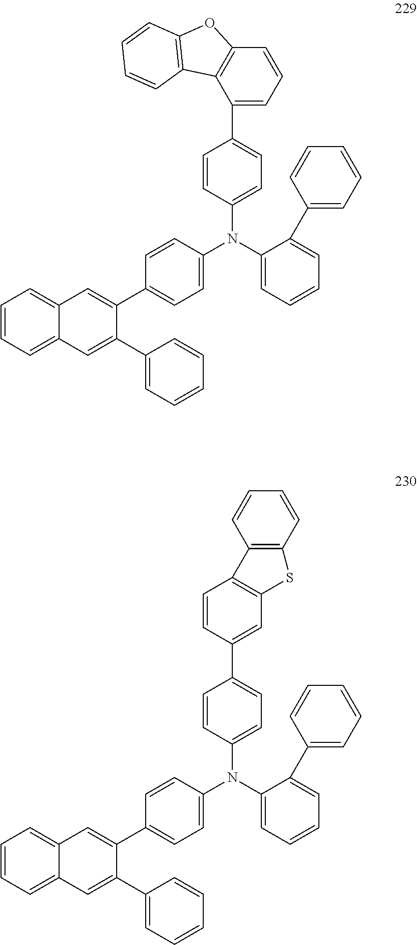 [SMILES: c1ccc(-c2cc3ccccc3cc2-c2ccc(N(c3ccc(-c4cccc5oc6ccccc6c45)cc3)c3ccccc3-c3ccccc3)cc2)cc1 ; c1ccc(-c2cc3ccccc3cc2-c2ccc(N(c3ccc(-c4ccc5c(c4)sc4ccccc45)cc3)c3ccccc3-c3ccccc3)cc2)cc1]

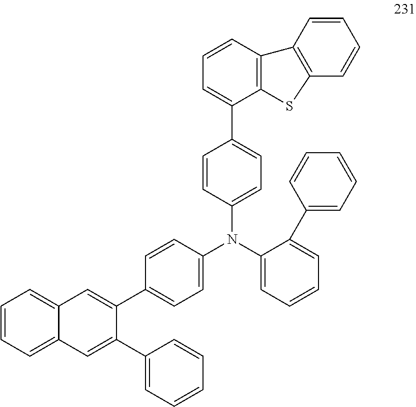 [SMILES: c1ccc(-c2cc3ccccc3cc2-c2ccc(N(c3ccc(-c4cccc5c4sc4ccccc45)cc3)c3ccccc3-c3ccccc3)cc2)cc1]

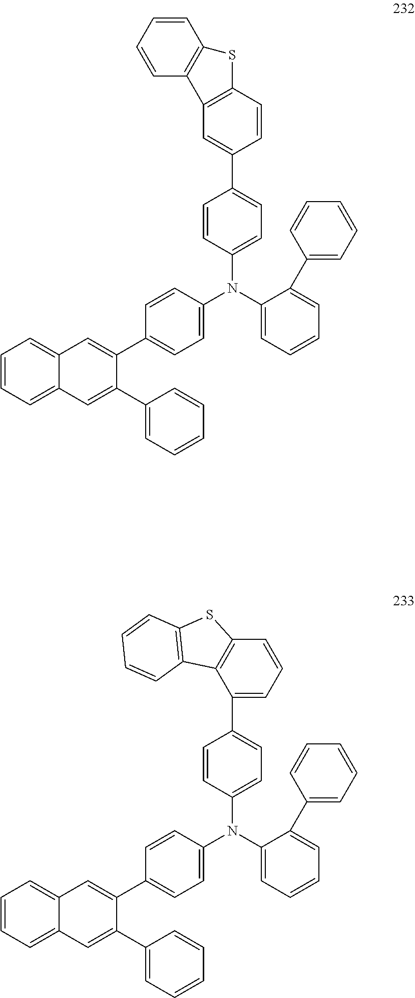 [SMILES: c1ccc(-c2cc3ccccc3cc2-c2ccc(N(c3ccc(-c4ccc5sc6ccccc6c5c4)cc3)c3ccccc3-c3ccccc3)cc2)cc1 ; c1ccc(-c2cc3ccccc3cc2-c2ccc(N(c3ccc(-c4cccc5sc6ccccc6c45)cc3)c3ccccc3-c3ccccc3)cc2)cc1]

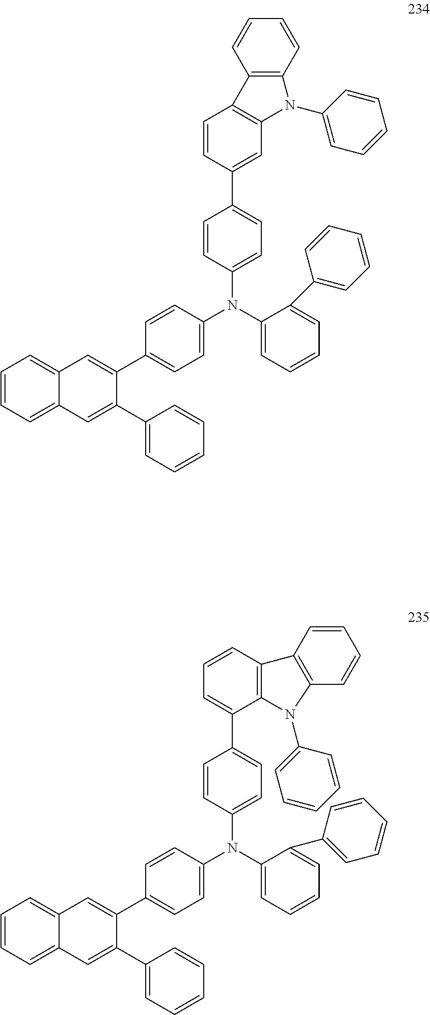 [SMILES: c1ccc(-c2cc3ccccc3cc2-c2ccc(N(c3ccc(-c4ccc5c6ccccc6n(-c6ccccc6)c5c4)cc3)c3ccccc3-c3ccccc3)cc2)cc1 ; c1ccc(-c2cc3ccccc3cc2-c2ccc(N(c3ccc(-c4cccc5c6ccccc6n(-c6ccccc6)c45)cc3)c3ccccc3-c3ccccc3)cc2)cc1]

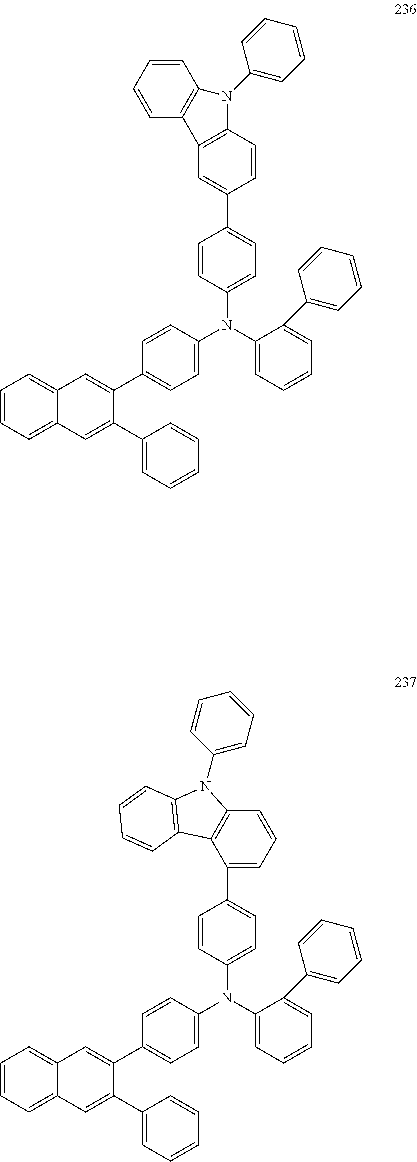 [SMILES: c1ccc(-c2cc3ccccc3cc2-c2ccc(N(c3ccc(-c4ccc5c(c4)c4ccccc4n5-c4ccccc4)cc3)c3ccccc3-c3ccccc3)cc2)cc1 ; c1ccc(-c2cc3ccccc3cc2-c2ccc(N(c3ccc(-c4cccc5c4c4ccccc4n5-c4ccccc4)cc3)c3ccccc3-c3ccccc3)cc2)cc1]

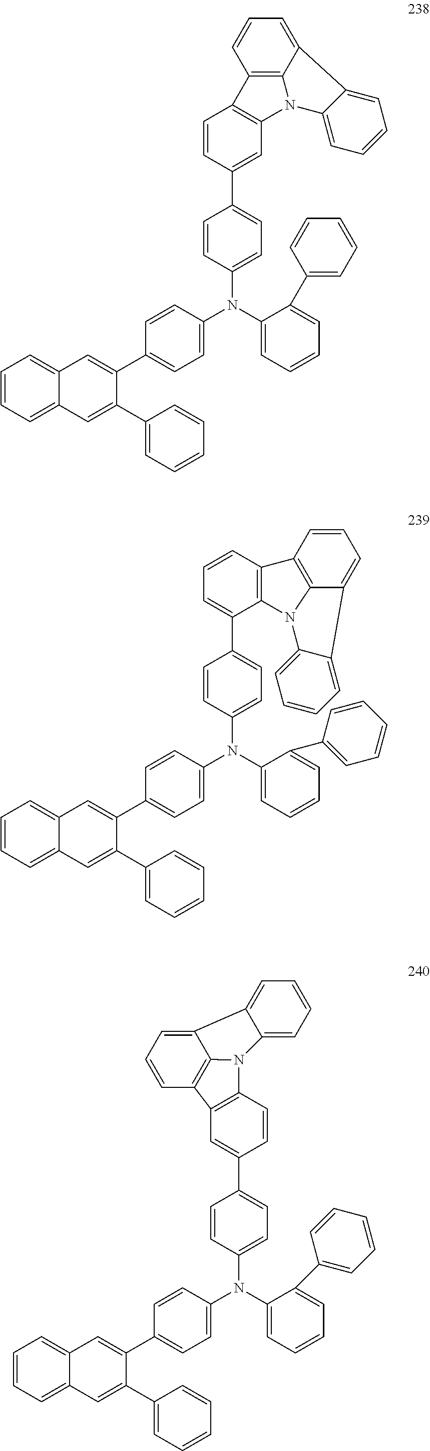 [SMILES: c1ccc(-c2cc3ccccc3cc2-c2ccc(N(c3ccc(-c4ccc5c6cccc7c8ccccc8n(c5c4)c76)cc3)c3ccccc3-c3ccccc3)cc2)cc1 ; c1ccc(-c2cc3ccccc3cc2-c2ccc(N(c3ccc(-c4cccc5c6cccc7c8ccccc8n(c45)c76)cc3)c3ccccc3-c3ccccc3)cc2)cc1 ; c1ccc(-c2cc3ccccc3cc2-c2ccc(N(c3ccc(-c4ccc5c(c4)c4cccc6c7ccccc7n5c64)cc3)c3ccccc3-c3ccccc3)cc2)cc1]

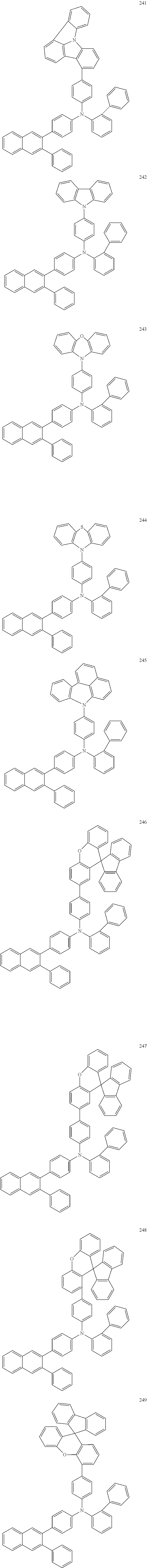

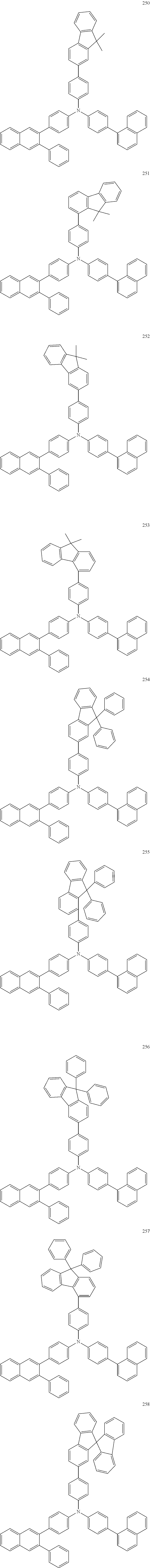

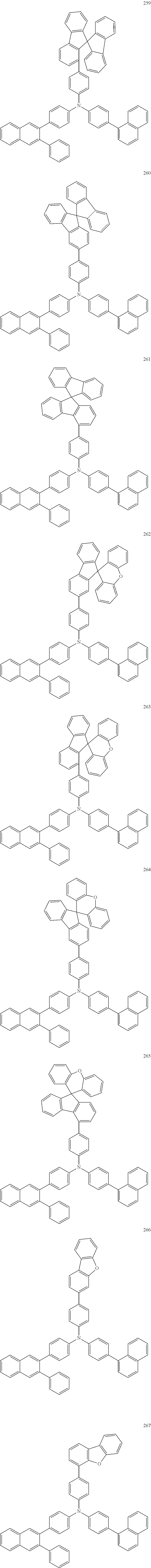

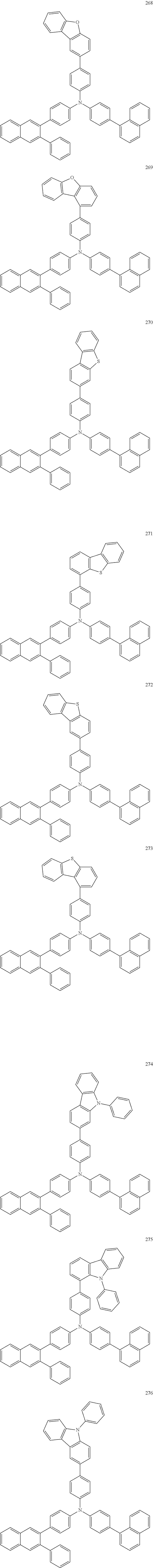

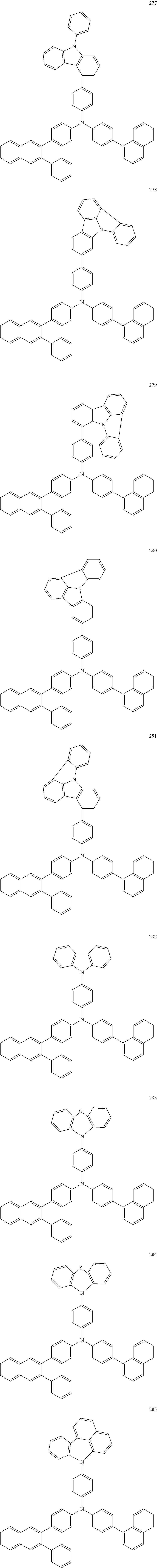

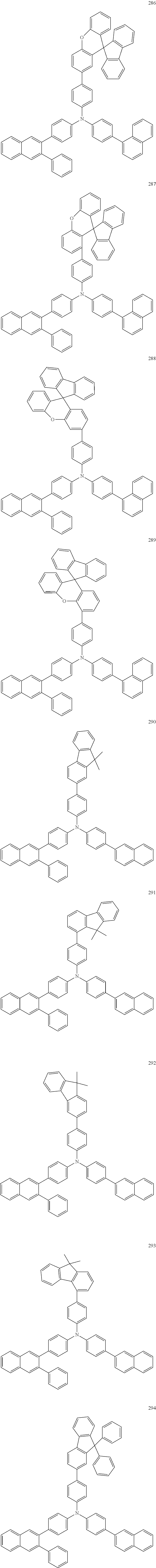

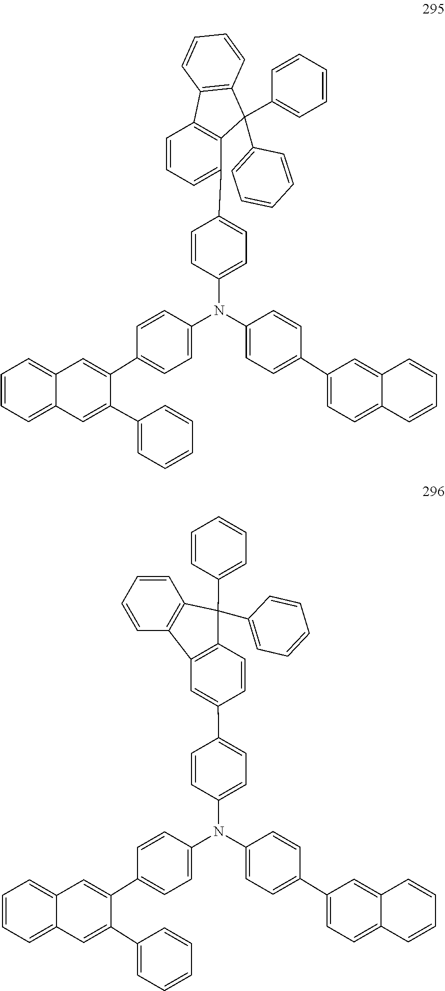 [SMILES: c1ccc(-c2cc3ccccc3cc2-c2ccc(N(c3ccc(-c4ccc5ccccc5c4)cc3)c3ccc(-c4cccc5c4C(c4ccccc4)(c4ccccc4)c4ccccc4-5)cc3)cc2)cc1 ; c1ccc(-c2cc3ccccc3cc2-c2ccc(N(c3ccc(-c4ccc5c(c4)-c4ccccc4C5(c4ccccc4)c4ccccc4)cc3)c3ccc(-c4ccc5ccccc5c4)cc3)cc2)cc1]

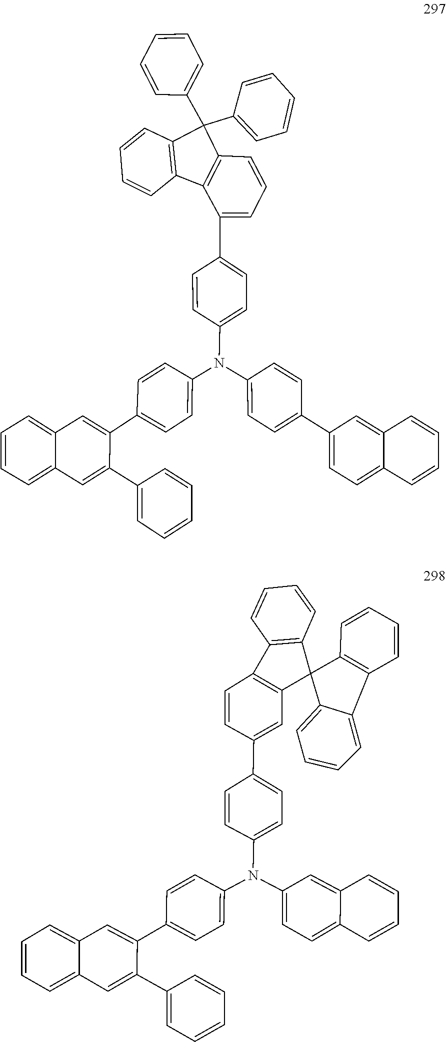 [SMILES: c1ccc(-c2cc3ccccc3cc2-c2ccc(N(c3ccc(-c4ccc5ccccc5c4)cc3)c3ccc(-c4cccc5c4-c4ccccc4C5(c4ccccc4)c4ccccc4)cc3)cc2)cc1 ; c1ccc(-c2cc3ccccc3cc2-c2ccc(N(c3ccc(-c4ccc5c(c4)C4(c6ccccc6-c6ccccc64)c4ccccc4-5)cc3)c3ccc4ccccc4c3)cc2)cc1]

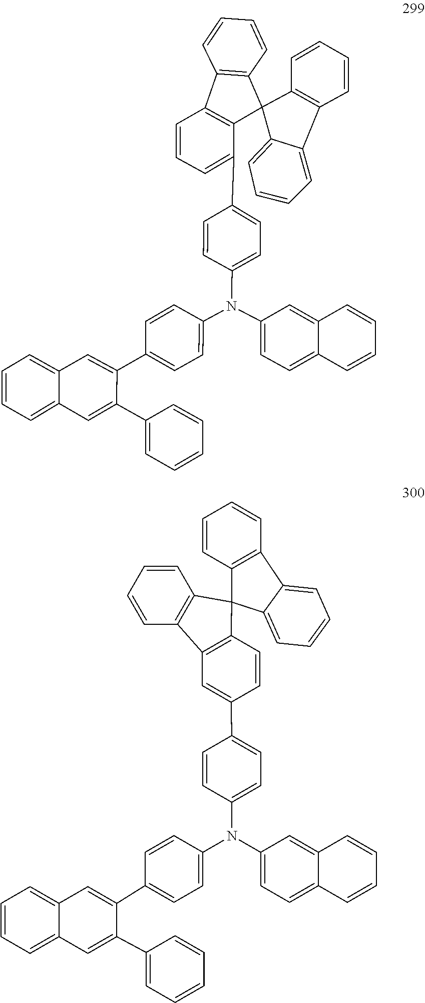 [SMILES: c1ccc(-c2cc3ccccc3cc2-c2ccc(N(c3ccc(-c4cccc5c4C4(c6ccccc6-c6ccccc64)c4ccccc4-5)cc3)c3ccc4ccccc4c3)cc2)cc1 ; c1ccc(-c2cc3ccccc3cc2-c2ccc(N(c3ccc(-c4ccc5c(c4)-c4ccccc4C54c5ccccc5-c5ccccc54)cc3)c3ccc4ccccc4c3)cc2)cc1]

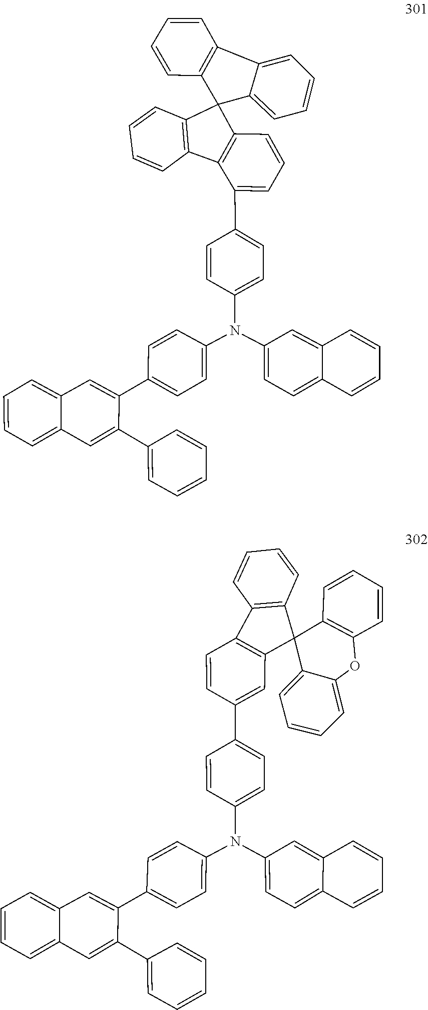 [SMILES: c1ccc(-c2cc3ccccc3cc2-c2ccc(N(c3ccc(-c4cccc5c4-c4ccccc4C54c5ccccc5-c5ccccc54)cc3)c3ccc4ccccc4c3)cc2)cc1 ; c1ccc(-c2cc3ccccc3cc2-c2ccc(N(c3ccc(-c4ccc5c(c4)C4(c6ccccc6Oc6ccccc64)c4ccccc4-5)cc3)c3ccc4ccccc4c3)cc2)cc1]

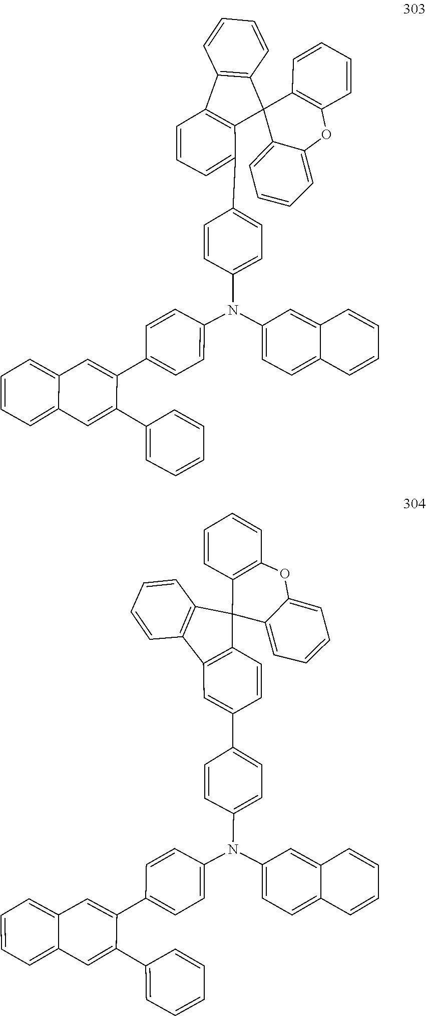 [SMILES: c1ccc(-c2cc3ccccc3cc2-c2ccc(N(c3ccc(-c4cccc5c4C4(c6ccccc6Oc6ccccc64)c4ccccc4-5)cc3)c3ccc4ccccc4c3)cc2)cc1 ; c1ccc(-c2cc3ccccc3cc2-c2ccc(N(c3ccc(-c4ccc5c(c4)-c4ccccc4C54c5ccccc5Oc5ccccc54)cc3)c3ccc4ccccc4c3)cc2)cc1]

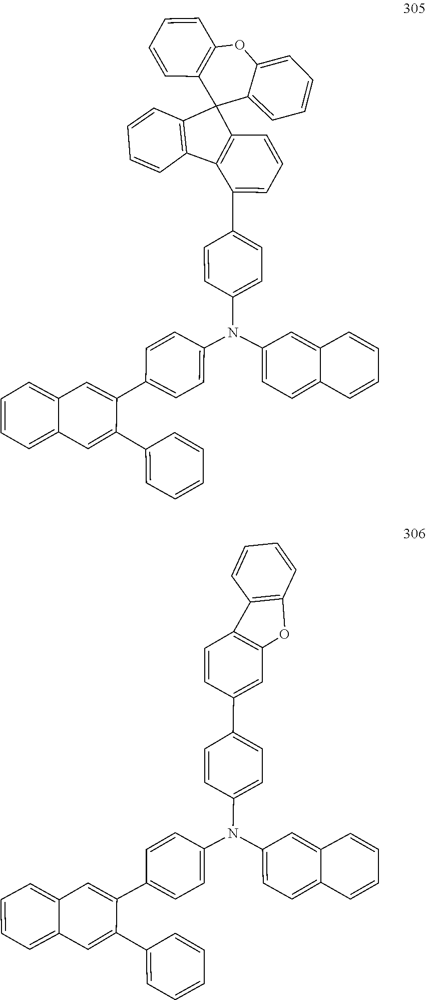 [SMILES: c1ccc(-c2cc3ccccc3cc2-c2ccc(N(c3ccc(-c4cccc5c4-c4ccccc4C54c5ccccc5Oc5ccccc54)cc3)c3ccc4ccccc4c3)cc2)cc1 ; c1ccc(-c2cc3ccccc3cc2-c2ccc(N(c3ccc(-c4ccc5c(c4)oc4ccccc45)cc3)c3ccc4ccccc4c3)cc2)cc1]

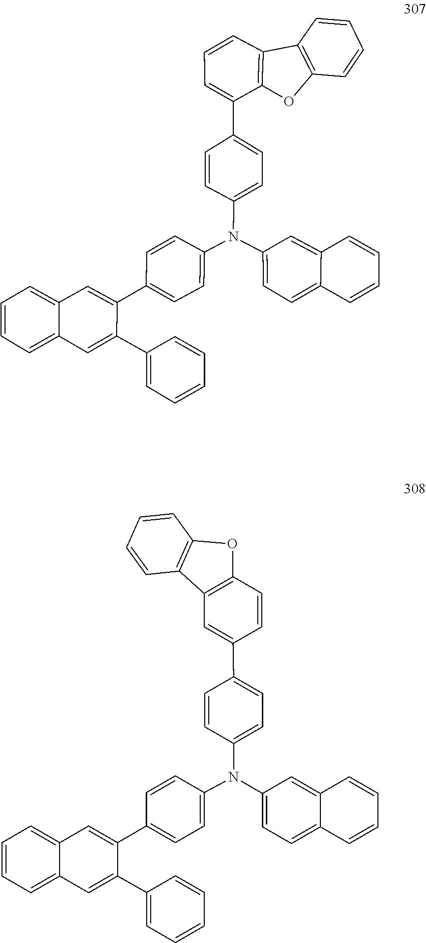 [SMILES: c1ccc(-c2cc3ccccc3cc2-c2ccc(N(c3ccc(-c4cccc5c4oc4ccccc45)cc3)c3ccc4ccccc4c3)cc2)cc1 ; c1ccc(-c2cc3ccccc3cc2-c2ccc(N(c3ccc(-c4ccc5oc6ccccc6c5c4)cc3)c3ccc4ccccc4c3)cc2)cc1]

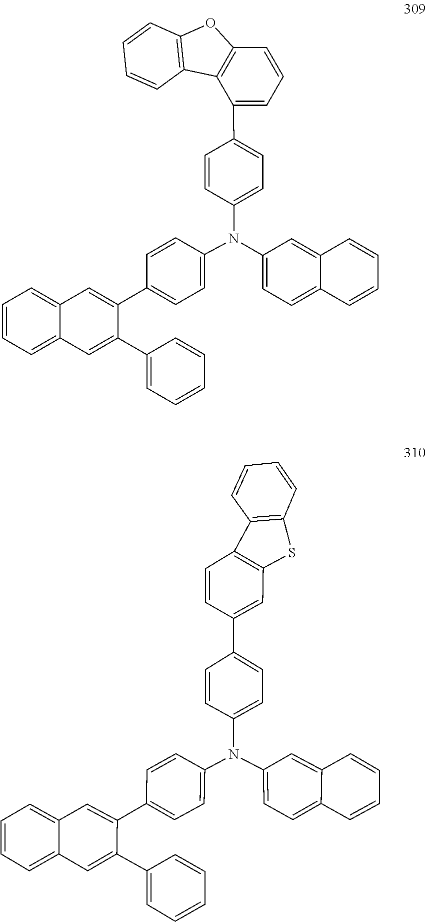 [SMILES: c1ccc(-c2cc3ccccc3cc2-c2ccc(N(c3ccc(-c4cccc5oc6ccccc6c45)cc3)c3ccc4ccccc4c3)cc2)cc1 ; c1ccc(-c2cc3ccccc3cc2-c2ccc(N(c3ccc(-c4ccc5c(c4)sc4ccccc45)cc3)c3ccc4ccccc4c3)cc2)cc1]

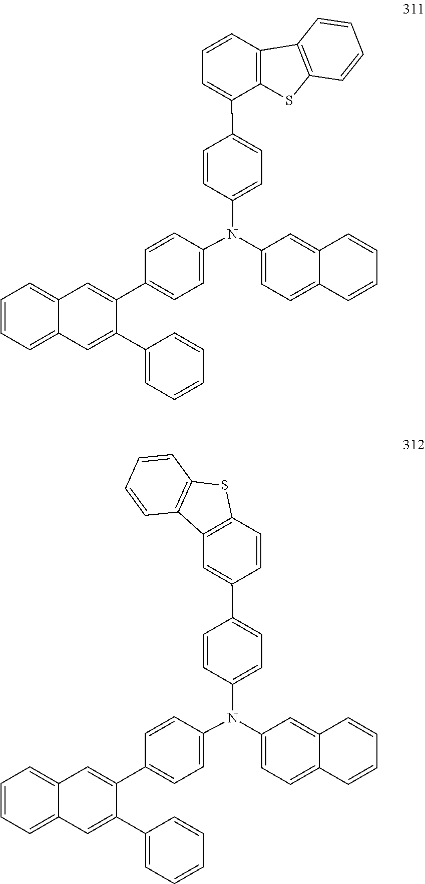 [SMILES: c1ccc(-c2cc3ccccc3cc2-c2ccc(N(c3ccc(-c4cccc5c4sc4ccccc45)cc3)c3ccc4ccccc4c3)cc2)cc1 ; c1ccc(-c2cc3ccccc3cc2-c2ccc(N(c3ccc(-c4ccc5sc6ccccc6c5c4)cc3)c3ccc4ccccc4c3)cc2)cc1]

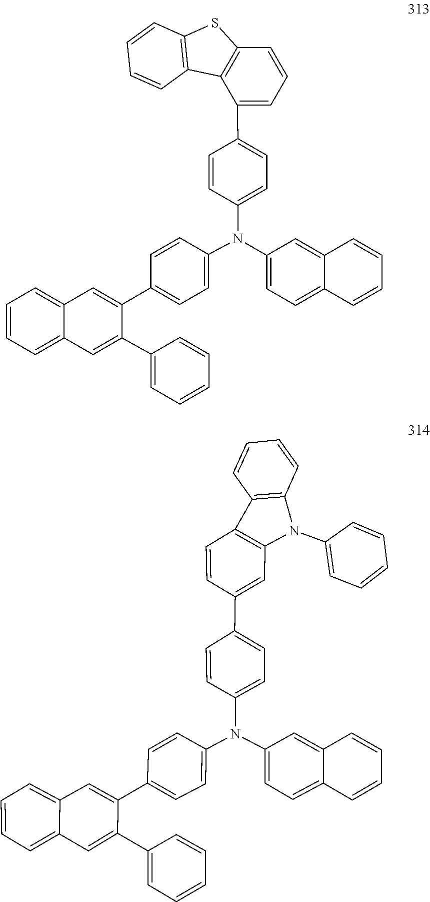 [SMILES: c1ccc(-c2cc3ccccc3cc2-c2ccc(N(c3ccc(-c4cccc5sc6ccccc6c45)cc3)c3ccc4ccccc4c3)cc2)cc1 ; c1ccc(-c2cc3ccccc3cc2-c2ccc(N(c3ccc(-c4ccc5c6ccccc6n(-c6ccccc6)c5c4)cc3)c3ccc4ccccc4c3)cc2)cc1]

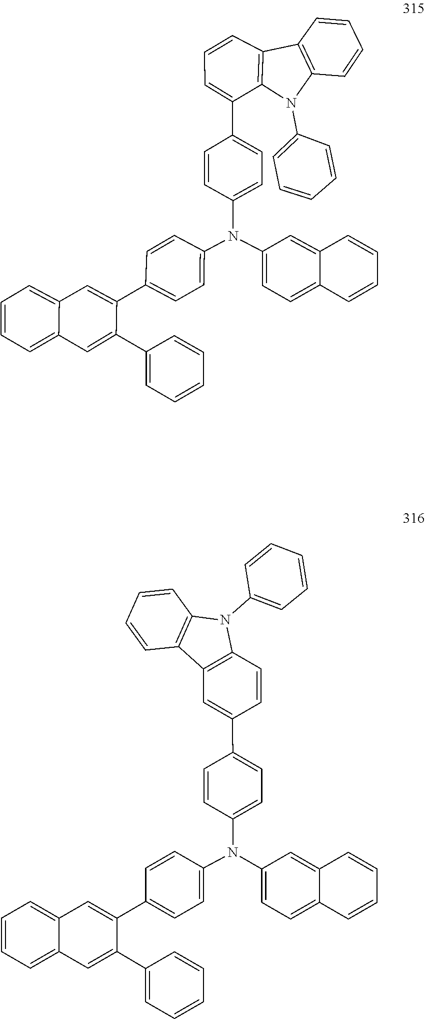 [SMILES: c1ccc(-c2cc3ccccc3cc2-c2ccc(N(c3ccc(-c4cccc5c6ccccc6n(-c6ccccc6)c45)cc3)c3ccc4ccccc4c3)cc2)cc1 ; c1ccc(-c2cc3ccccc3cc2-c2ccc(N(c3ccc(-c4ccc5c(c4)c4ccccc4n5-c4ccccc4)cc3)c3ccc4ccccc4c3)cc2)cc1]

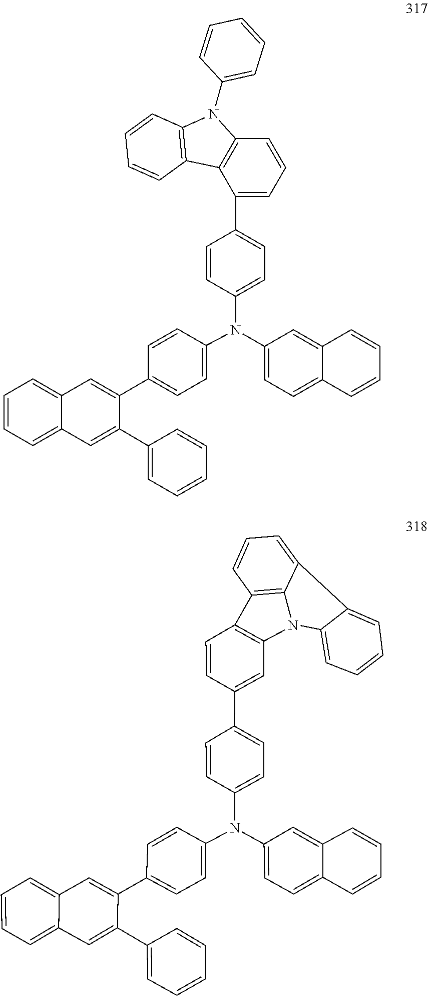 [SMILES: c1ccc(-c2cc3ccccc3cc2-c2ccc(N(c3ccc(-c4cccc5c4c4ccccc4n5-c4ccccc4)cc3)c3ccc4ccccc4c3)cc2)cc1 ; c1ccc(-c2cc3ccccc3cc2-c2ccc(N(c3ccc(-c4ccc5c6cccc7c8ccccc8n(c5c4)c76)cc3)c3ccc4ccccc4c3)cc2)cc1]

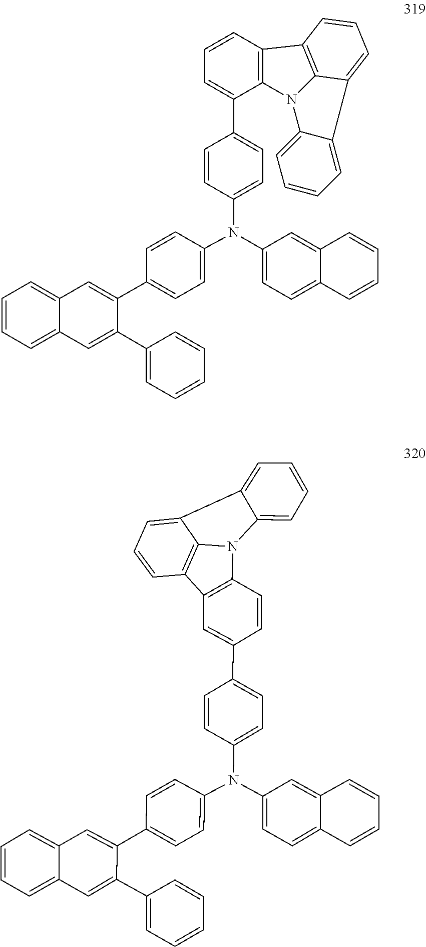 [SMILES: c1ccc(-c2cc3ccccc3cc2-c2ccc(N(c3ccc(-c4cccc5c6cccc7c8ccccc8n(c45)c76)cc3)c3ccc4ccccc4c3)cc2)cc1 ; c1ccc(-c2cc3ccccc3cc2-c2ccc(N(c3ccc(-c4ccc5c(c4)c4cccc6c7ccccc7n5c64)cc3)c3ccc4ccccc4c3)cc2)cc1]

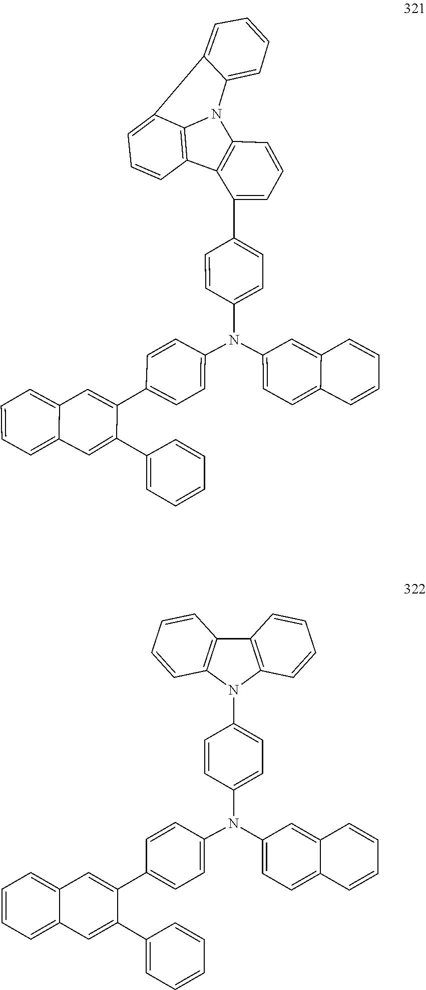 [SMILES: c1ccc(-c2cc3ccccc3cc2-c2ccc(N(c3ccc(-c4cccc5c4c4cccc6c7ccccc7n5c64)cc3)c3ccc4ccccc4c3)cc2)cc1 ; c1ccc(-c2cc3ccccc3cc2-c2ccc(N(c3ccc(-n4c5ccccc5c5ccccc54)cc3)c3ccc4ccccc4c3)cc2)cc1]

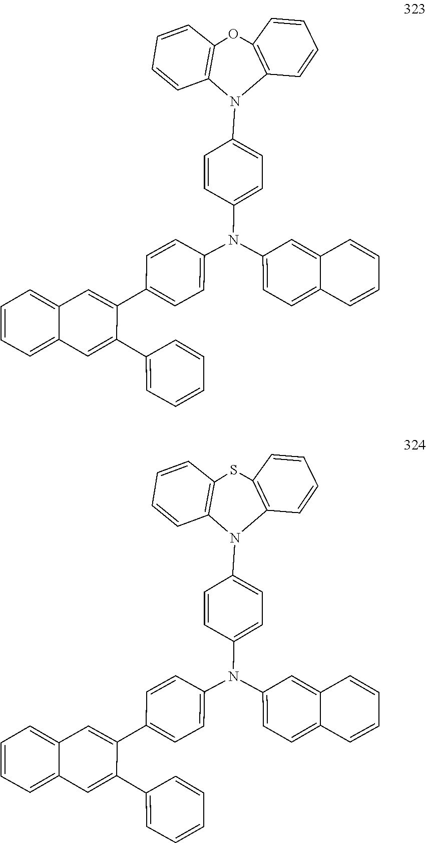 [SMILES: c1ccc(-c2cc3ccccc3cc2-c2ccc(N(c3ccc(N4c5ccccc5Oc5ccccc54)cc3)c3ccc4ccccc4c3)cc2)cc1 ; c1ccc(-c2cc3ccccc3cc2-c2ccc(N(c3ccc(N4c5ccccc5Sc5ccccc54)cc3)c3ccc4ccccc4c3)cc2)cc1]

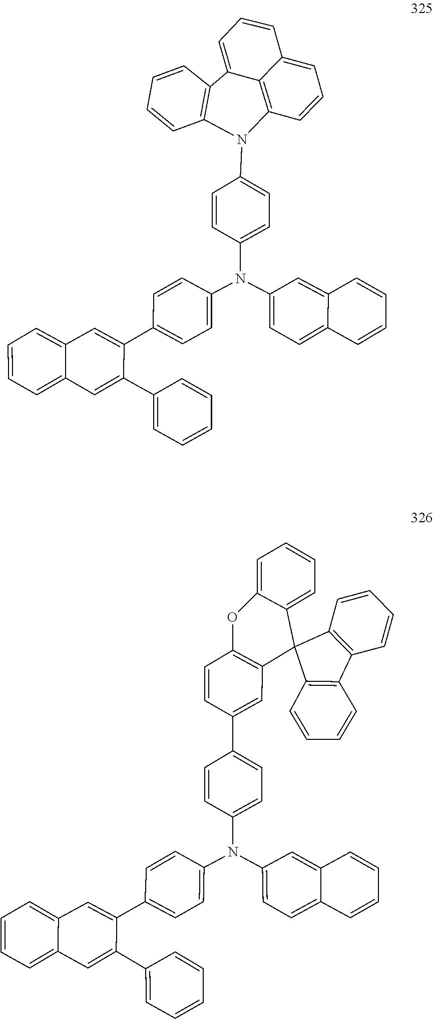 [SMILES: c1ccc(-c2cc3ccccc3cc2-c2ccc(N(c3ccc(N4c5ccccc5-c5cccc6cccc4c56)cc3)c3ccc4ccccc4c3)cc2)cc1 ; c1ccc(-c2cc3ccccc3cc2-c2ccc(N(c3ccc(-c4ccc5c(c4)C4(c6ccccc6O5)c5ccccc5-c5ccccc54)cc3)c3ccc4ccccc4c3)cc2)cc1]

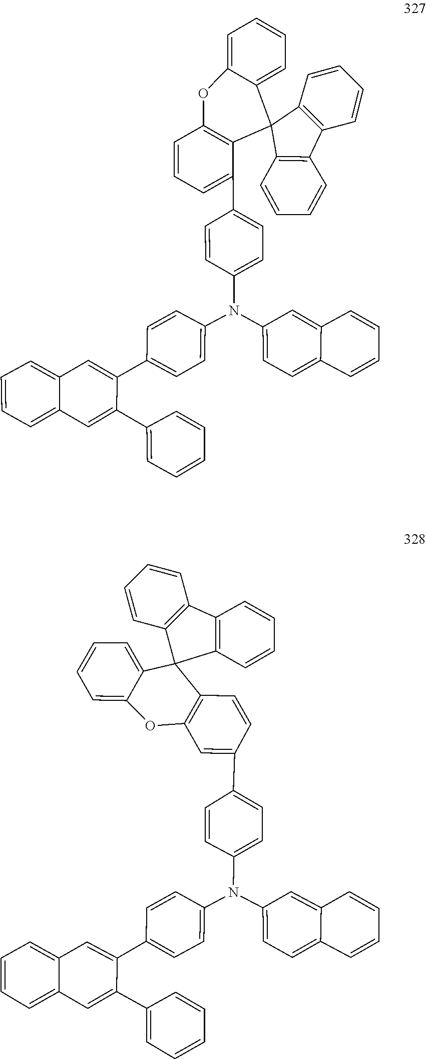 [SMILES: c1ccc(-c2cc3ccccc3cc2-c2ccc(N(c3ccc(-c4cccc5c4C4(c6ccccc6O5)c5ccccc5-c5ccccc54)cc3)c3ccc4ccccc4c3)cc2)cc1 ; c1ccc(-c2cc3ccccc3cc2-c2ccc(N(c3ccc(-c4ccc5c(c4)Oc4ccccc4C54c5ccccc5-c5ccccc54)cc3)c3ccc4ccccc4c3)cc2)cc1]

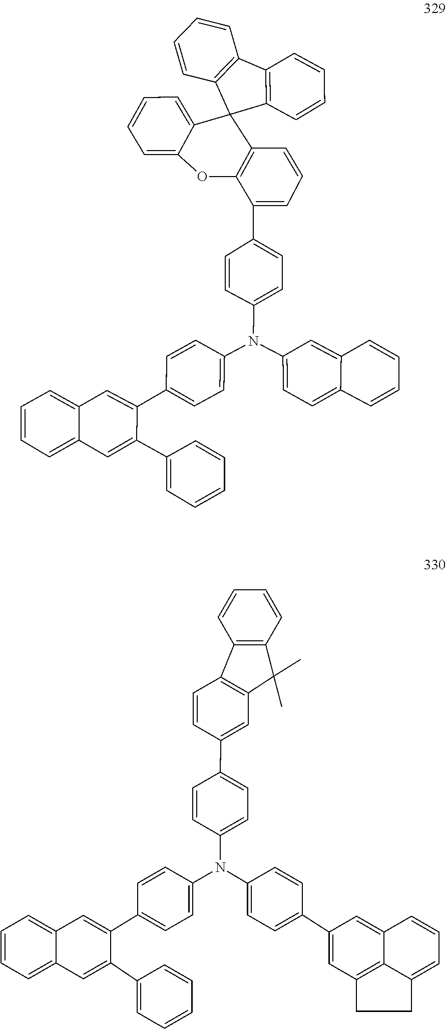 [SMILES: c1ccc(-c2cc3ccccc3cc2-c2ccc(N(c3ccc(-c4cccc5c4Oc4ccccc4C54c5ccccc5-c5ccccc54)cc3)c3ccc4ccccc4c3)cc2)cc1 ; CC1(C)c2ccccc2-c2ccc(-c3ccc(N(c4ccc(-c5cc6c7c(cccc7c5)CC6)cc4)c4ccc(-c5cc6ccccc6cc5-c5ccccc5)cc4)cc3)cc21]

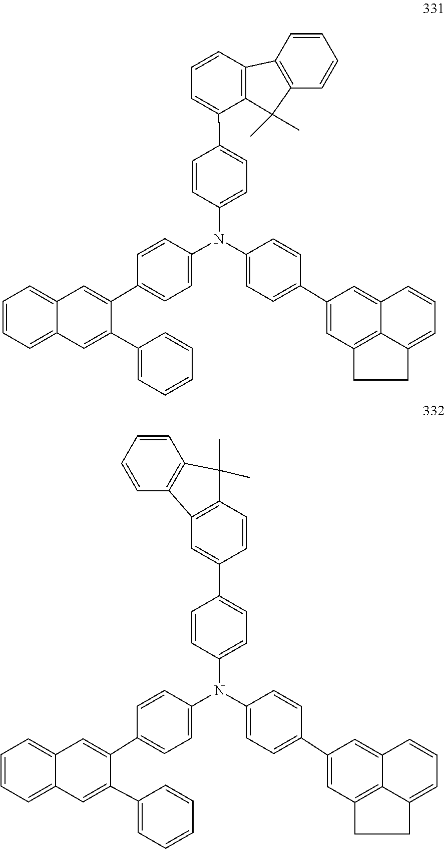

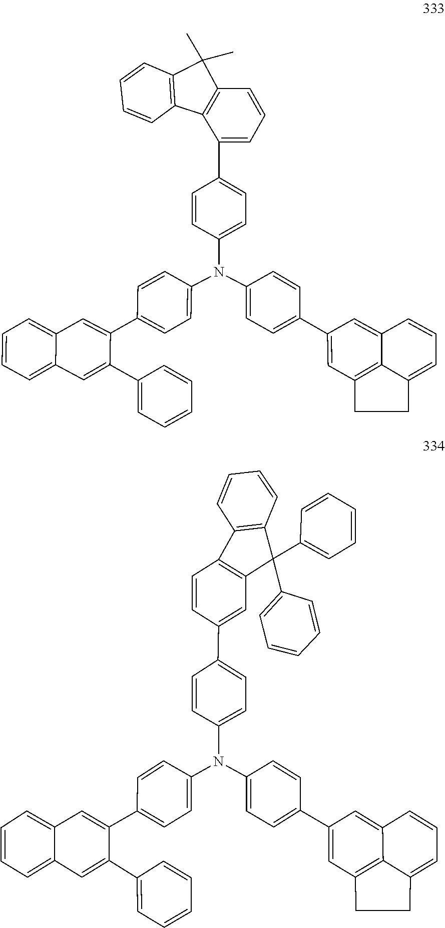 [SMILES: CC1(C)c2ccccc2-c2c(-c3ccc(N(c4ccc(-c5cc6c7c(cccc7c5)CC6)cc4)c4ccc(-c5cc6ccccc6cc5-c5ccccc5)cc4)cc3)cccc21 ; c1ccc(-c2cc3ccccc3cc2-c2ccc(N(c3ccc(-c4ccc5c(c4)C(c4ccccc4)(c4ccccc4)c4ccccc4-5)cc3)c3ccc(-c4cc5c6c(cccc6c4)CC5)cc3)cc2)cc1]

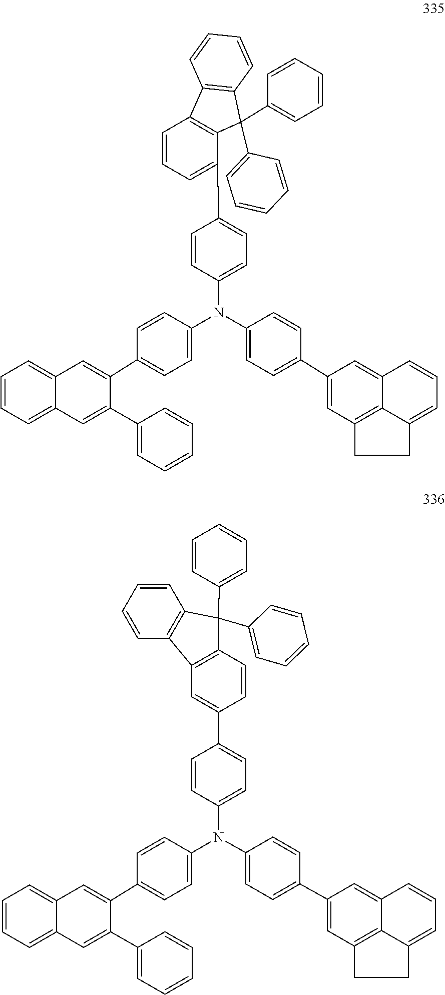 [SMILES: c1ccc(-c2cc3ccccc3cc2-c2ccc(N(c3ccc(-c4cc5c6c(cccc6c4)CC5)cc3)c3ccc(-c4cccc5c4C(c4ccccc4)(c4ccccc4)c4ccccc4-5)cc3)cc2)cc1 ; c1ccc(-c2cc3ccccc3cc2-c2ccc(N(c3ccc(-c4ccc5c(c4)-c4ccccc4C5(c4ccccc4)c4ccccc4)cc3)c3ccc(-c4cc5c6c(cccc6c4)CC5)cc3)cc2)cc1]

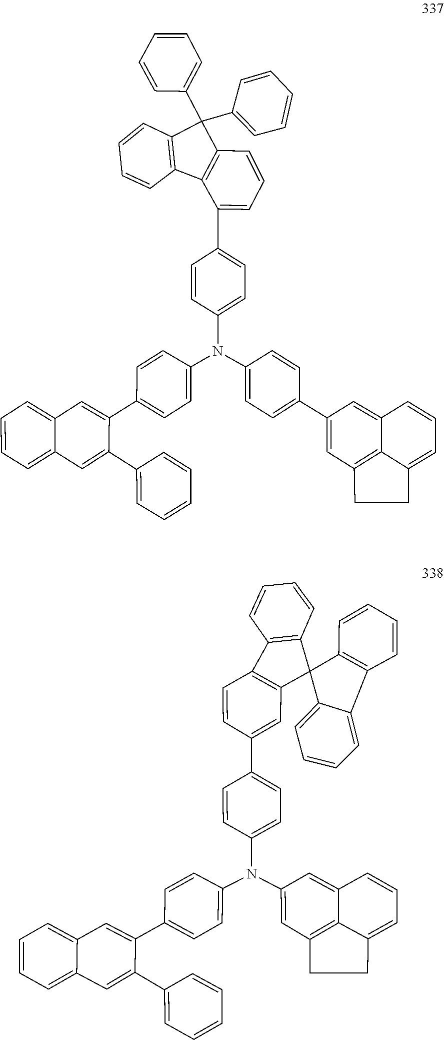 [SMILES: c1ccc(-c2cc3ccccc3cc2-c2ccc(N(c3ccc(-c4cc5c6c(cccc6c4)CC5)cc3)c3ccc(-c4cccc5c4-c4ccccc4C5(c4ccccc4)c4ccccc4)cc3)cc2)cc1 ; c1ccc(-c2cc3ccccc3cc2-c2ccc(N(c3ccc(-c4ccc5c(c4)C4(c6ccccc6-c6ccccc64)c4ccccc4-5)cc3)c3cc4c5c(cccc5c3)CC4)cc2)cc1]

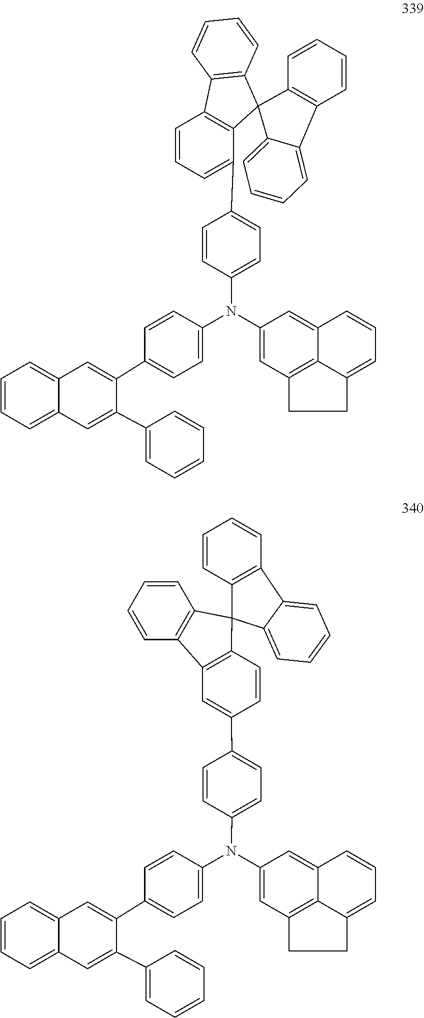 [SMILES: c1ccc(-c2cc3ccccc3cc2-c2ccc(N(c3ccc(-c4cccc5c4C4(c6ccccc6-c6ccccc64)c4ccccc4-5)cc3)c3cc4c5c(cccc5c3)CC4)cc2)cc1 ; c1ccc(-c2cc3ccccc3cc2-c2ccc(N(c3ccc(-c4ccc5c(c4)-c4ccccc4C54c5ccccc5-c5ccccc54)cc3)c3cc4c5c(cccc5c3)CC4)cc2)cc1]

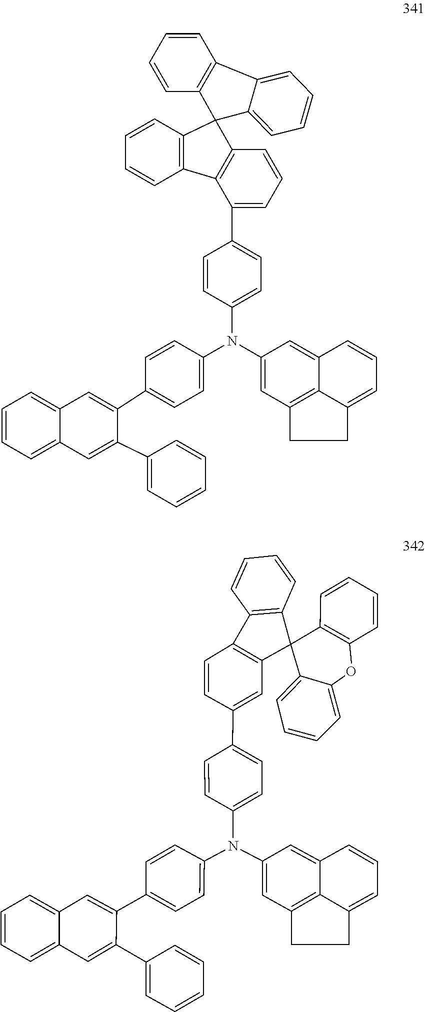 [SMILES: c1ccc(-c2cc3ccccc3cc2-c2ccc(N(c3ccc(-c4cccc5c4-c4ccccc4C54c5ccccc5-c5ccccc54)cc3)c3cc4c5c(cccc5c3)CC4)cc2)cc1 ; c1ccc(-c2cc3ccccc3cc2-c2ccc(N(c3ccc(-c4ccc5c(c4)C4(c6ccccc6Oc6ccccc64)c4ccccc4-5)cc3)c3cc4c5c(cccc5c3)CC4)cc2)cc1]

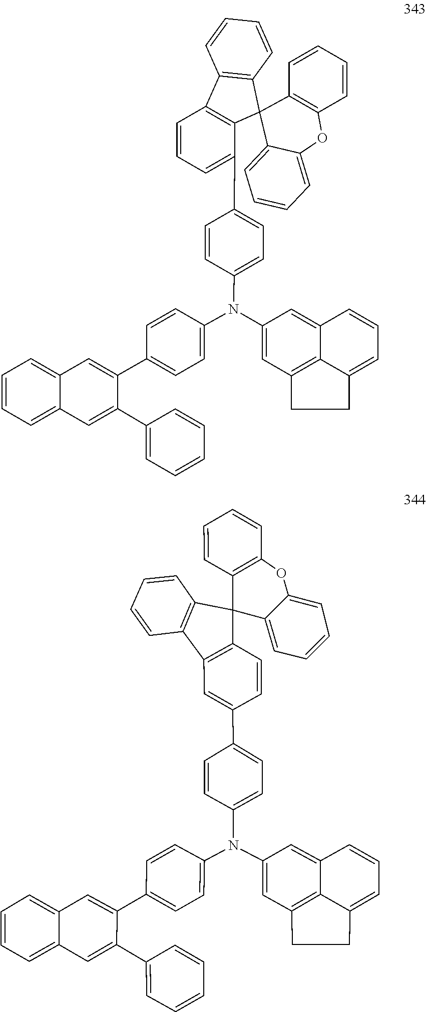 [SMILES: c1ccc(-c2cc3ccccc3cc2-c2ccc(N(c3ccc(-c4cccc5c4C4(c6ccccc6Oc6ccccc64)c4ccccc4-5)cc3)c3cc4c5c(cccc5c3)CC4)cc2)cc1 ; c1ccc(-c2cc3ccccc3cc2-c2ccc(N(c3ccc(-c4ccc5c(c4)-c4ccccc4C54c5ccccc5Oc5ccccc54)cc3)c3cc4c5c(cccc5c3)CC4)cc2)cc1]

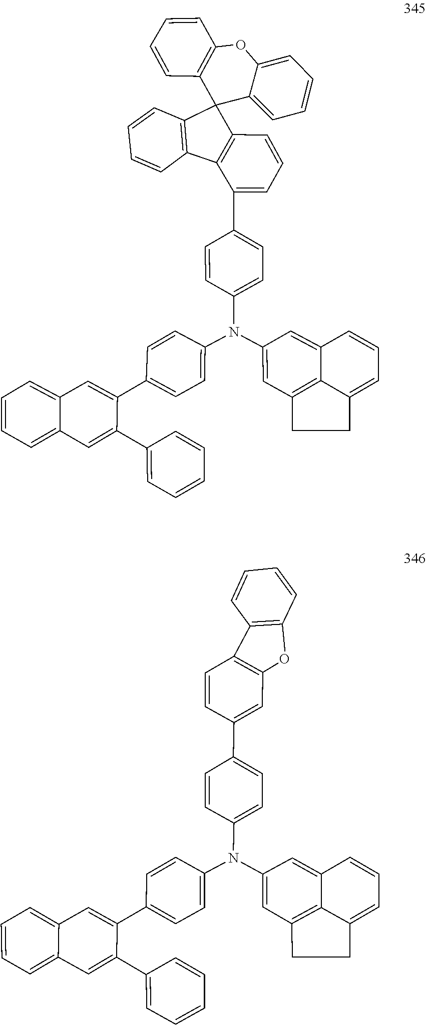 [SMILES: c1ccc(-c2cc3ccccc3cc2-c2ccc(N(c3ccc(-c4cccc5c4-c4ccccc4C54c5ccccc5Oc5ccccc54)cc3)c3cc4c5c(cccc5c3)CC4)cc2)cc1 ; c1ccc(-c2cc3ccccc3cc2-c2ccc(N(c3ccc(-c4ccc5c(c4)oc4ccccc45)cc3)c3cc4c5c(cccc5c3)CC4)cc2)cc1]

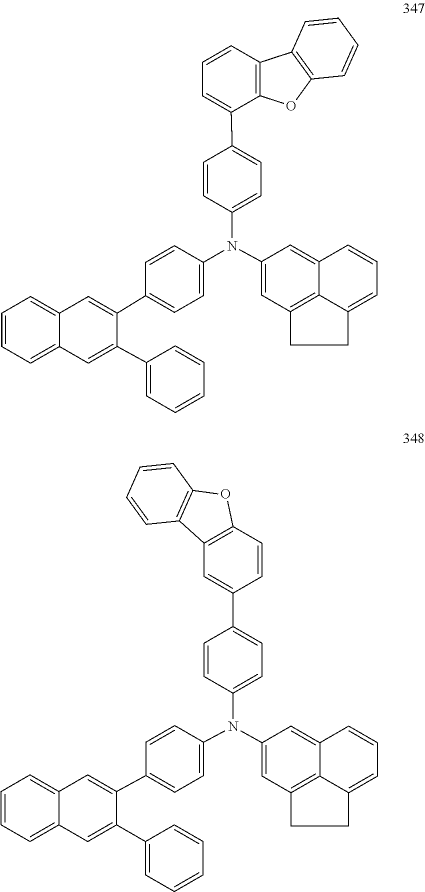 [SMILES: c1ccc(-c2cc3ccccc3cc2-c2ccc(N(c3ccc(-c4cccc5c4oc4ccccc45)cc3)c3cc4c5c(cccc5c3)CC4)cc2)cc1 ; c1ccc(-c2cc3ccccc3cc2-c2ccc(N(c3ccc(-c4ccc5oc6ccccc6c5c4)cc3)c3cc4c5c(cccc5c3)CC4)cc2)cc1]

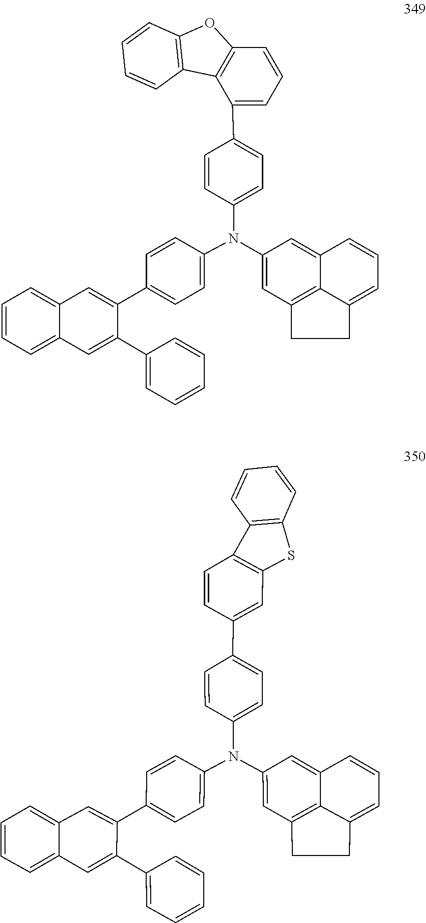 [SMILES: c1ccc(-c2cc3ccccc3cc2-c2ccc(N(c3ccc(-c4cccc5oc6ccccc6c45)cc3)c3cc4c5c(cccc5c3)CC4)cc2)cc1 ; c1ccc(-c2cc3ccccc3cc2-c2ccc(N(c3ccc(-c4ccc5c(c4)sc4ccccc45)cc3)c3cc4c5c(cccc5c3)CC4)cc2)cc1]

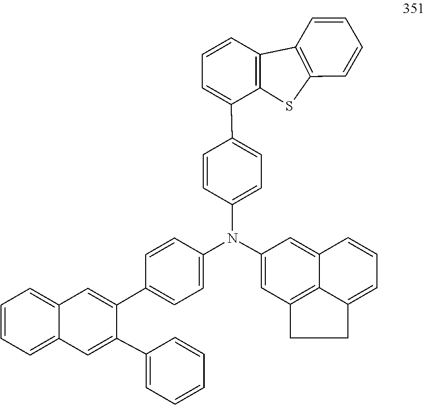 [SMILES: c1ccc(-c2cc3ccccc3cc2-c2ccc(N(c3ccc(-c4cccc5c4sc4ccccc45)cc3)c3cc4c5c(cccc5c3)CC4)cc2)cc1]

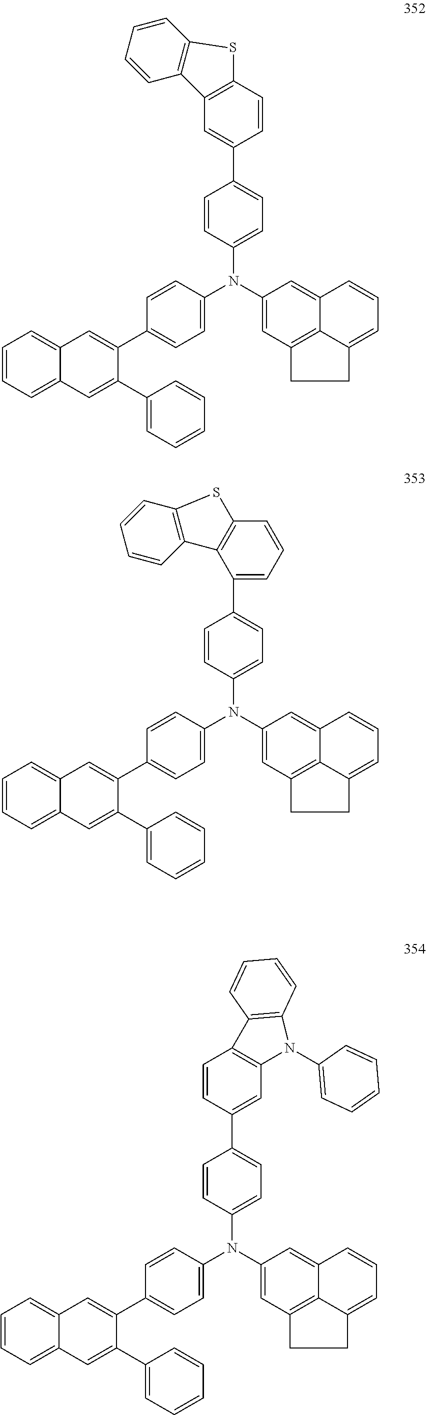 [SMILES: c1ccc(-c2cc3ccccc3cc2-c2ccc(N(c3ccc(-c4ccc5sc6ccccc6c5c4)cc3)c3cc4c5c(cccc5c3)CC4)cc2)cc1 ; c1ccc(-c2cc3ccccc3cc2-c2ccc(N(c3ccc(-c4cccc5sc6ccccc6c45)cc3)c3cc4c5c(cccc5c3)CC4)cc2)cc1 ; c1ccc(-c2cc3ccccc3cc2-c2ccc(N(c3ccc(-c4ccc5c6ccccc6n(-c6ccccc6)c5c4)cc3)c3cc4c5c(cccc5c3)CC4)cc2)cc1]

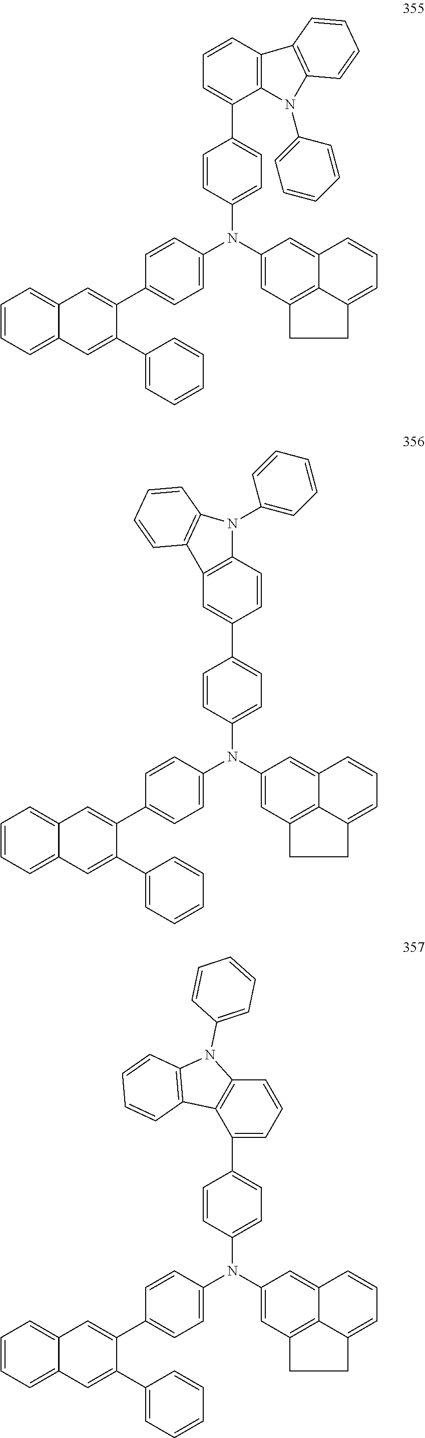 [SMILES: c1ccc(-c2cc3ccccc3cc2-c2ccc(N(c3ccc(-c4cccc5c6ccccc6n(-c6ccccc6)c45)cc3)c3cc4c5c(cccc5c3)CC4)cc2)cc1 ; c1ccc(-c2cc3ccccc3cc2-c2ccc(N(c3ccc(-c4ccc5c(c4)c4ccccc4n5-c4ccccc4)cc3)c3cc4c5c(cccc5c3)CC4)cc2)cc1 ; c1ccc(-c2cc3ccccc3cc2-c2ccc(N(c3ccc(-c4cccc5c4c4ccccc4n5-c4ccccc4)cc3)c3cc4c5c(cccc5c3)CC4)cc2)cc1]

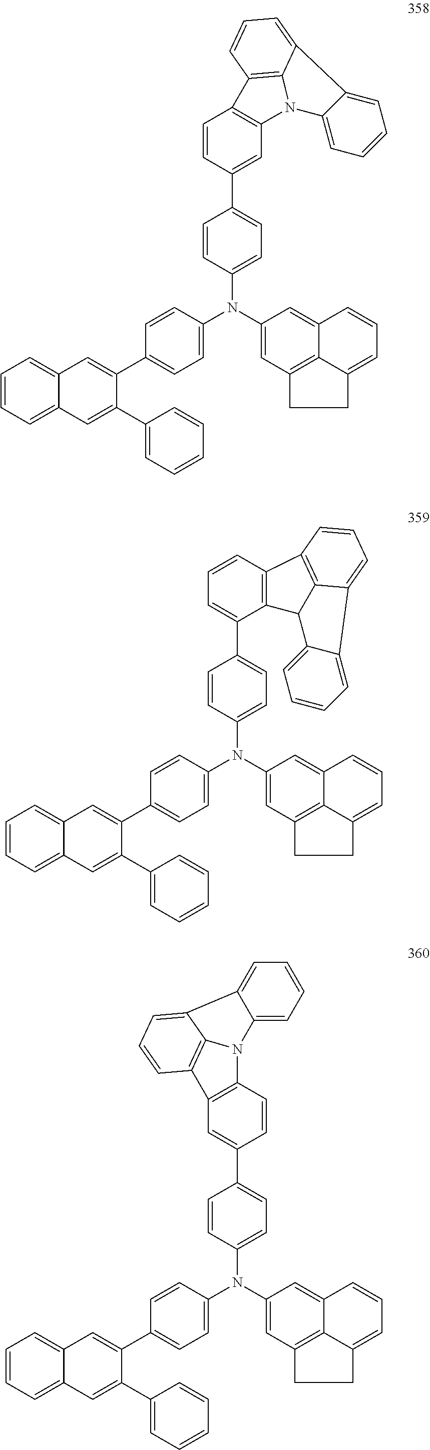 [SMILES: c1ccc(-c2cc3ccccc3cc2-c2ccc(N(c3ccc(-c4ccc5c6cccc7c8ccccc8n(c5c4)c76)cc3)c3cc4c5c(cccc5c3)CC4)cc2)cc1 ; c1ccc(-c2cc3ccccc3cc2-c2ccc(N(c3ccc(-c4cccc5c4C4c6ccccc6-c6cccc-5c64)cc3)c3cc4c5c(cccc5c3)CC4)cc2)cc1 ; c1ccc(-c2cc3ccccc3cc2-c2ccc(N(c3ccc(-c4ccc5c(c4)c4cccc6c7ccccc7n5c64)cc3)c3cc4c5c(cccc5c3)CC4)cc2)cc1]

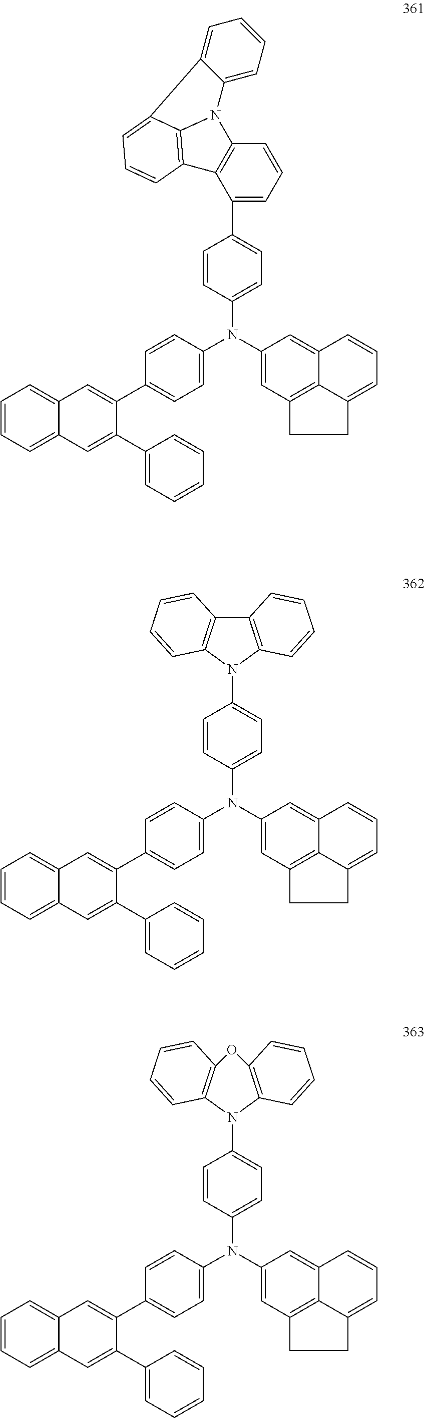 [SMILES: c1ccc(-c2cc3ccccc3cc2-c2ccc(N(c3ccc(-c4cccc5c4c4cccc6c7ccccc7n5c64)cc3)c3cc4c5c(cccc5c3)CC4)cc2)cc1 ; c1ccc(-c2cc3ccccc3cc2-c2ccc(N(c3ccc(-n4c5ccccc5c5ccccc54)cc3)c3cc4c5c(cccc5c3)CC4)cc2)cc1 ; c1ccc(-c2cc3ccccc3cc2-c2ccc(N(c3ccc(N4c5ccccc5Oc5ccccc54)cc3)c3cc4c5c(cccc5c3)CC4)cc2)cc1]

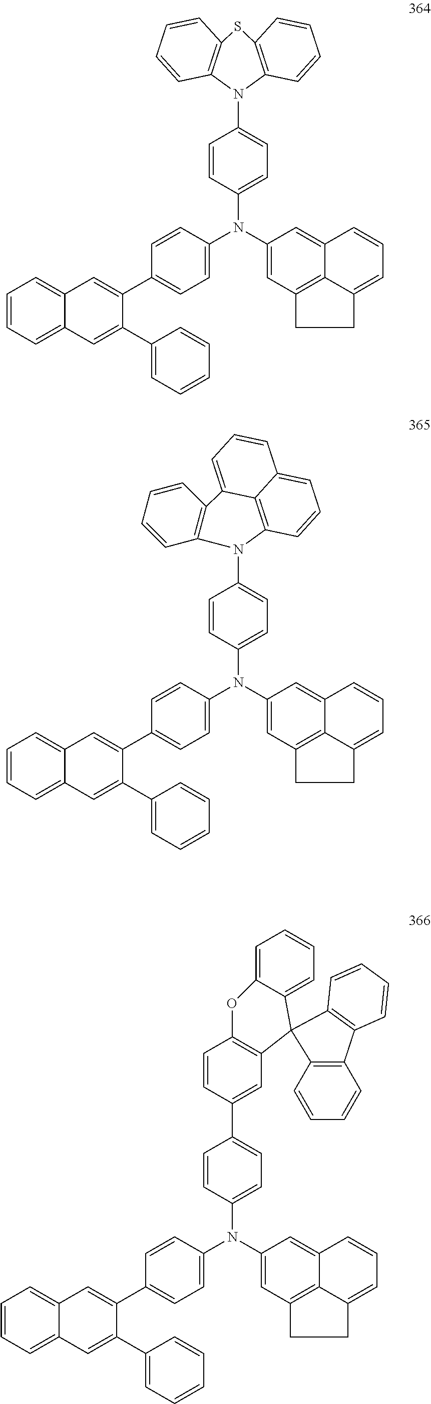 [SMILES: c1ccc(-c2cc3ccccc3cc2-c2ccc(N(c3ccc(N4c5ccccc5Sc5ccccc54)cc3)c3cc4c5c(cccc5c3)CC4)cc2)cc1 ; c1ccc(-c2cc3ccccc3cc2-c2ccc(N(c3ccc(N4c5ccccc5-c5cccc6cccc4c56)cc3)c3cc4c5c(cccc5c3)CC4)cc2)cc1 ; c1ccc(-c2cc3ccccc3cc2-c2ccc(N(c3ccc(-c4ccc5c(c4)C4(c6ccccc6O5)c5ccccc5-c5ccccc54)cc3)c3cc4c5c(cccc5c3)CC4)cc2)cc1]

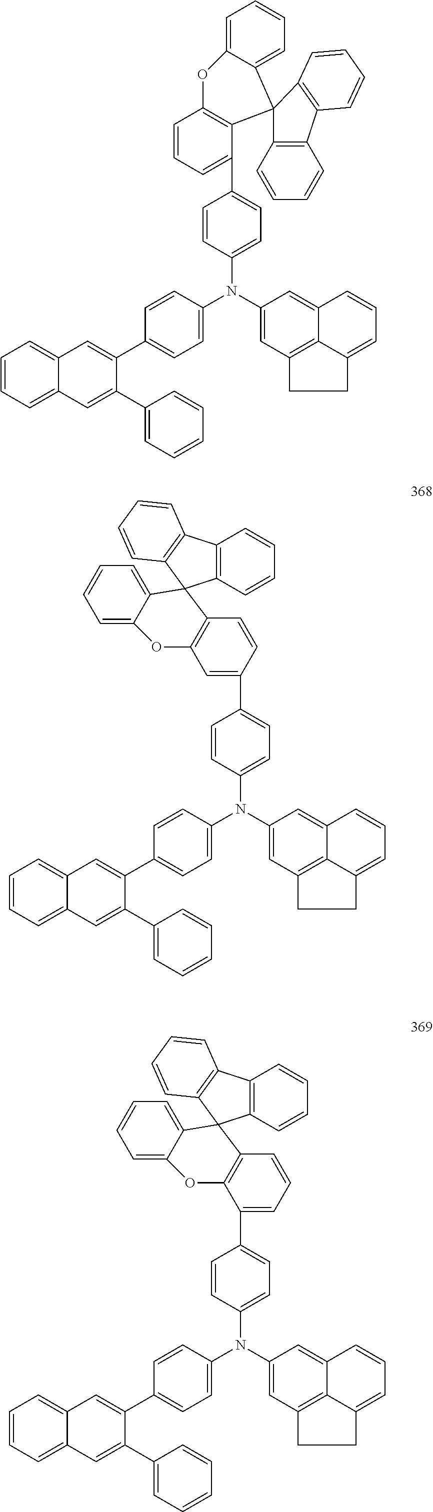

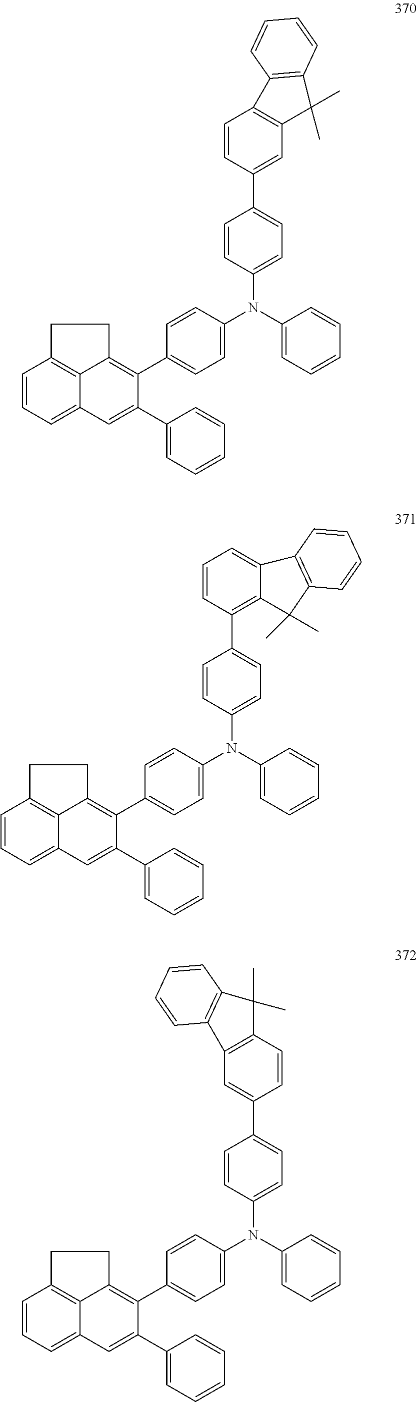 [SMILES: CC1(C)c2ccccc2-c2ccc(-c3ccc(N(c4ccccc4)c4ccc(-c5c(-c6ccccc6)cc6cccc7c6c5CC7)cc4)cc3)cc21 ; CC1(C)c2ccccc2-c2cccc(-c3ccc(N(c4ccccc4)c4ccc(-c5c(-c6ccccc6)cc6cccc7c6c5CC7)cc4)cc3)c21 ; CC1(C)c2ccccc2-c2cc(-c3ccc(N(c4ccccc4)c4ccc(-c5c(-c6ccccc6)cc6cccc7c6c5CC7)cc4)cc3)ccc21]

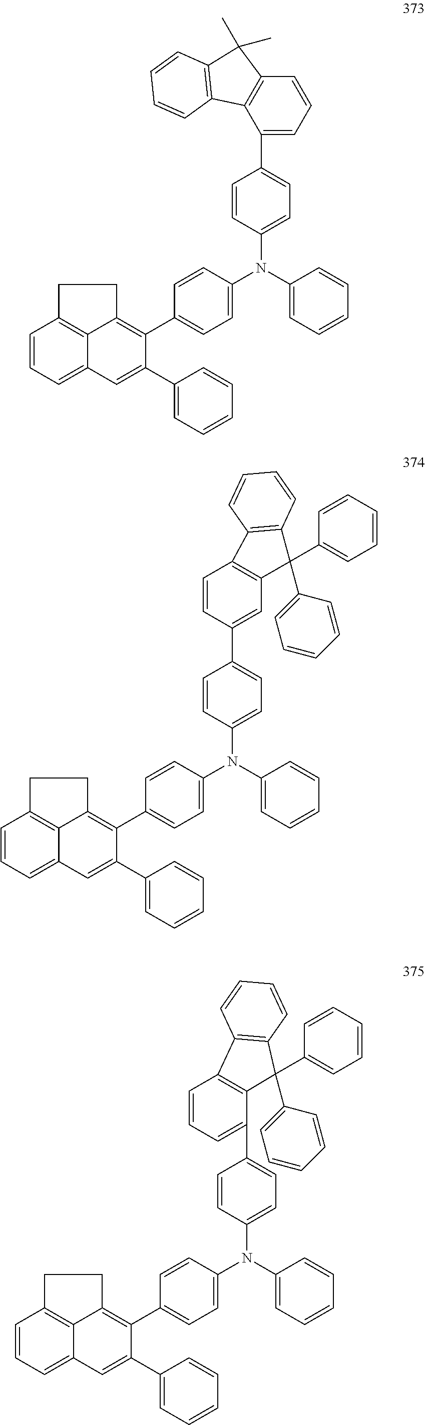

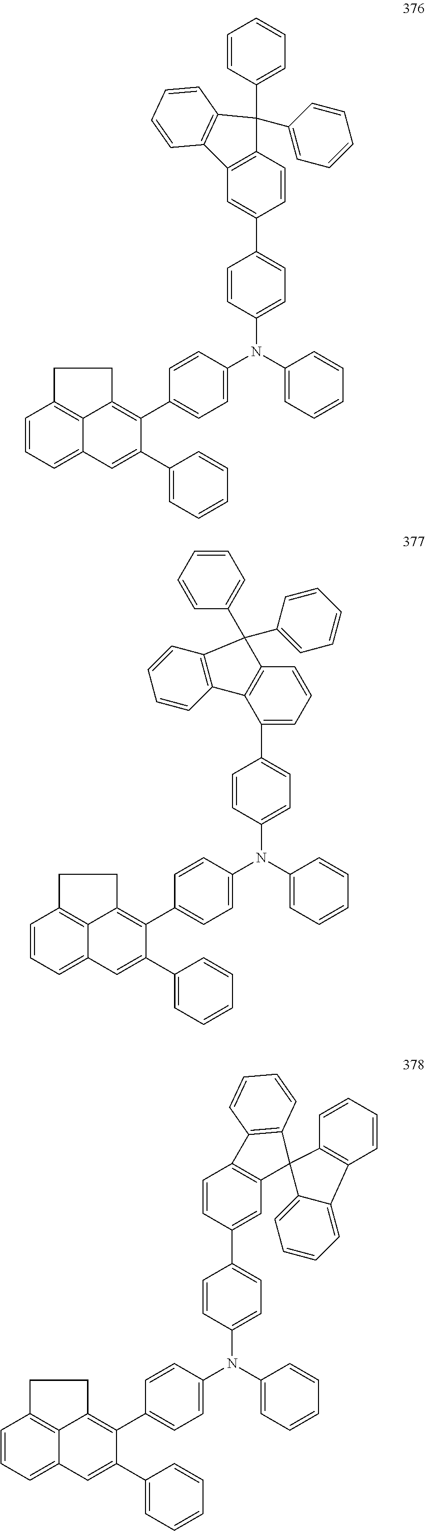

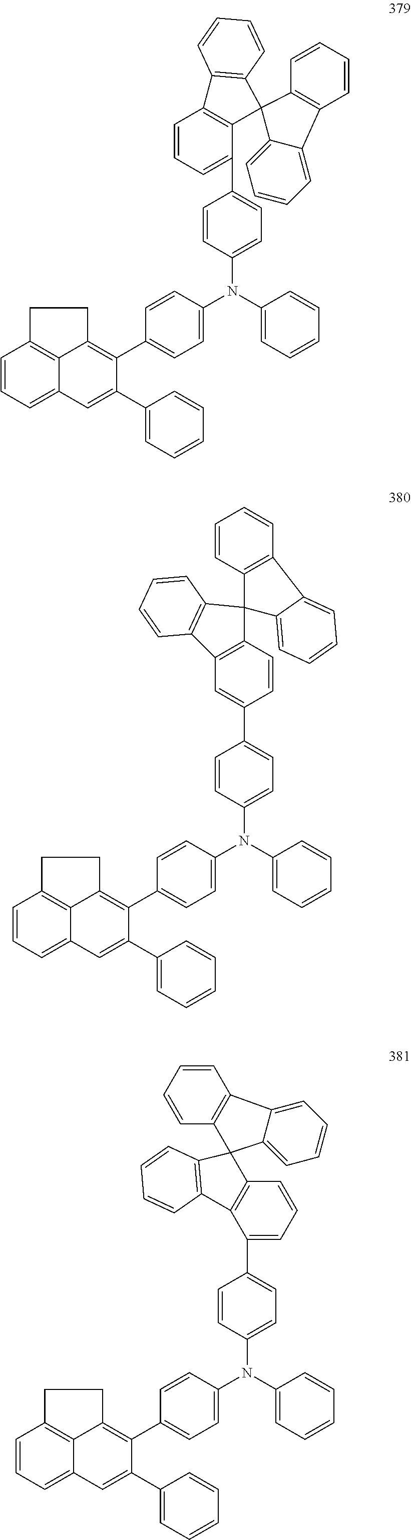

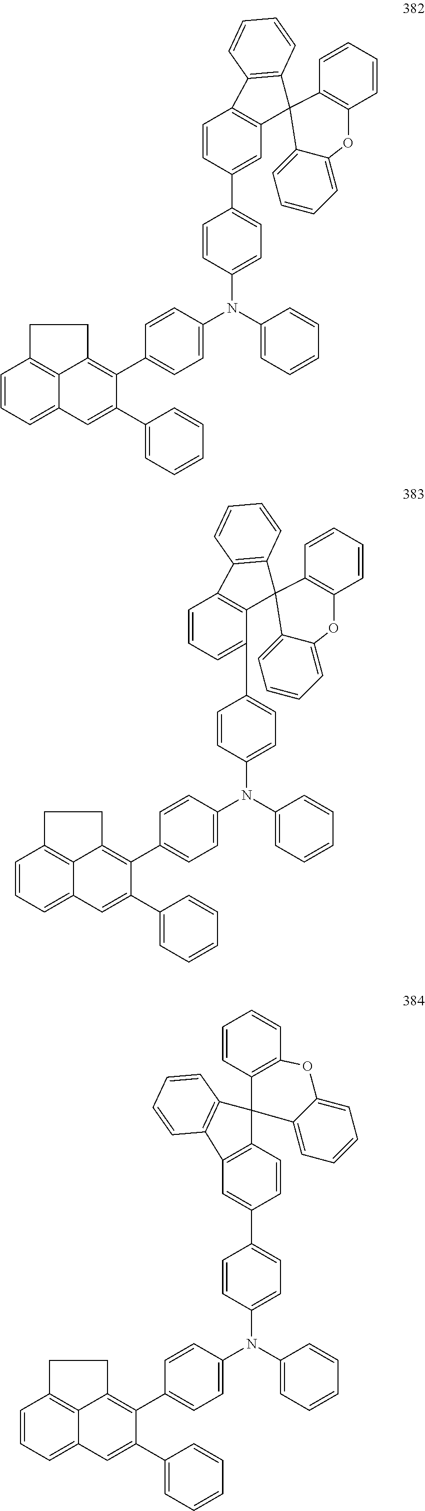 [SMILES: c1ccc(-c2cc3cccc4c3c(c2-c2ccc(N(c3ccccc3)c3ccc(-c5ccc6c(c5)C5(c7ccccc7Oc7ccccc75)c5ccccc5-6)cc3)cc2)CC4)cc1 ; c1ccc(-c2cc3cccc4c3c(c2-c2ccc(N(c3ccccc3)c3ccc(-c5cccc6c5C5(c7ccccc7Oc7ccccc75)c5ccccc5-6)cc3)cc2)CC4)cc1 ; c1ccc(-c2cc3cccc4c3c(c2-c2ccc(N(c3ccccc3)c3ccc(-c5ccc6c(c5)-c5ccccc5C65c6ccccc6Oc6ccccc65)cc3)cc2)CC4)cc1]

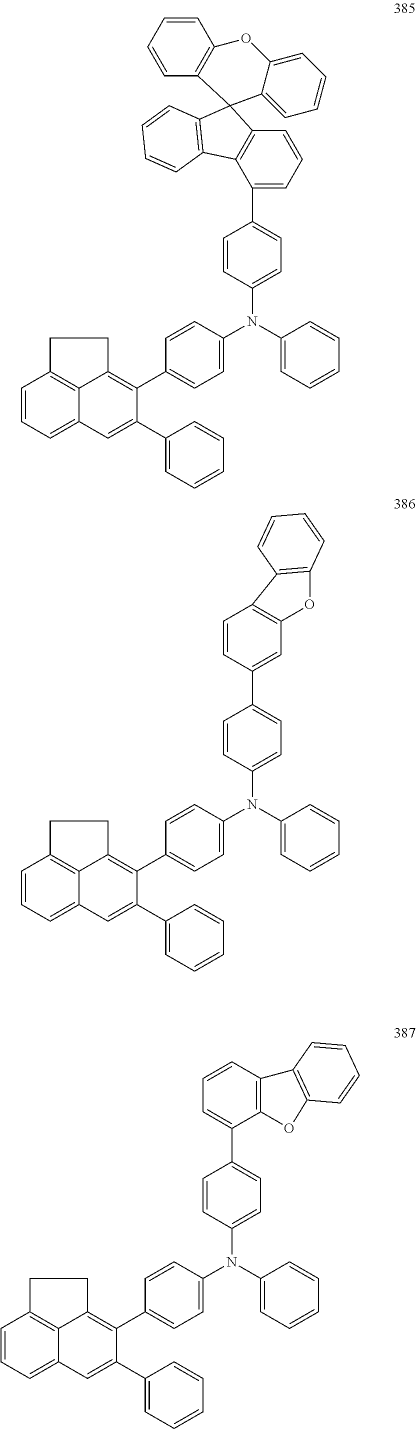 [SMILES: c1ccc(-c2cc3cccc4c3c(c2-c2ccc(N(c3ccccc3)c3ccc(-c5cccc6c5-c5ccccc5C65c6ccccc6Oc6ccccc65)cc3)cc2)CC4)cc1 ; c1ccc(-c2cc3cccc4c3c(c2-c2ccc(N(c3ccccc3)c3ccc(-c5ccc6c(c5)oc5ccccc56)cc3)cc2)CC4)cc1 ; c1ccc(-c2cc3cccc4c3c(c2-c2ccc(N(c3ccccc3)c3ccc(-c5cccc6c5oc5ccccc56)cc3)cc2)CC4)cc1]

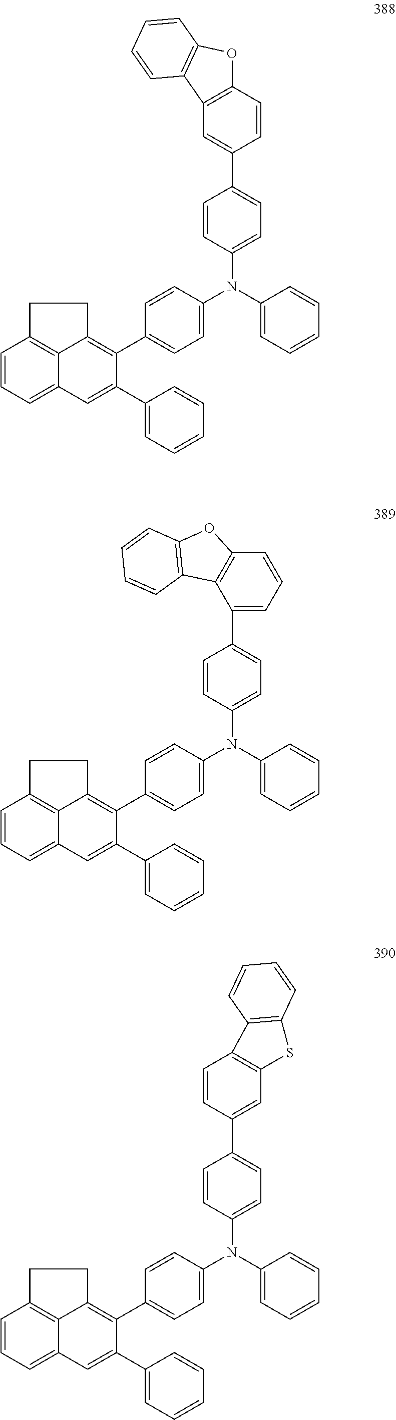

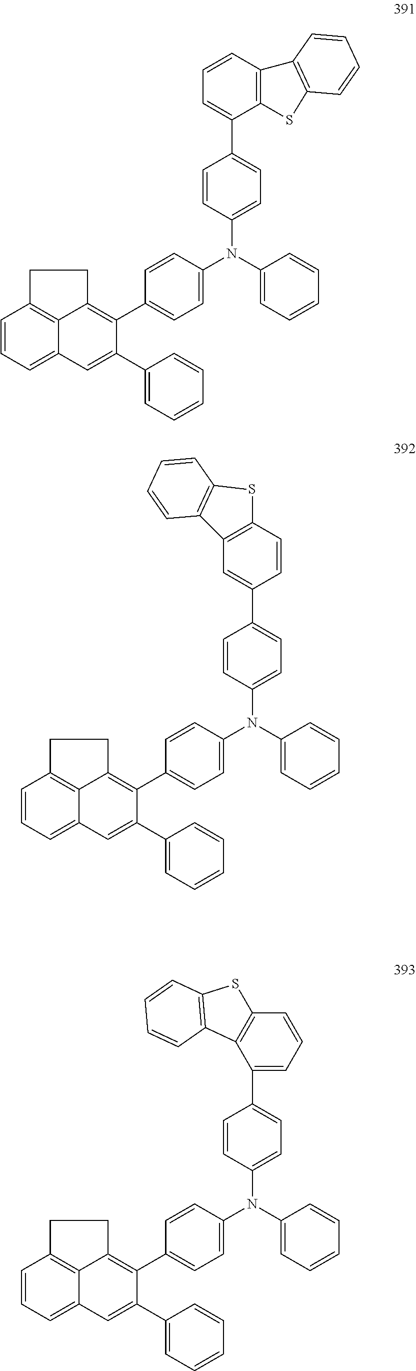

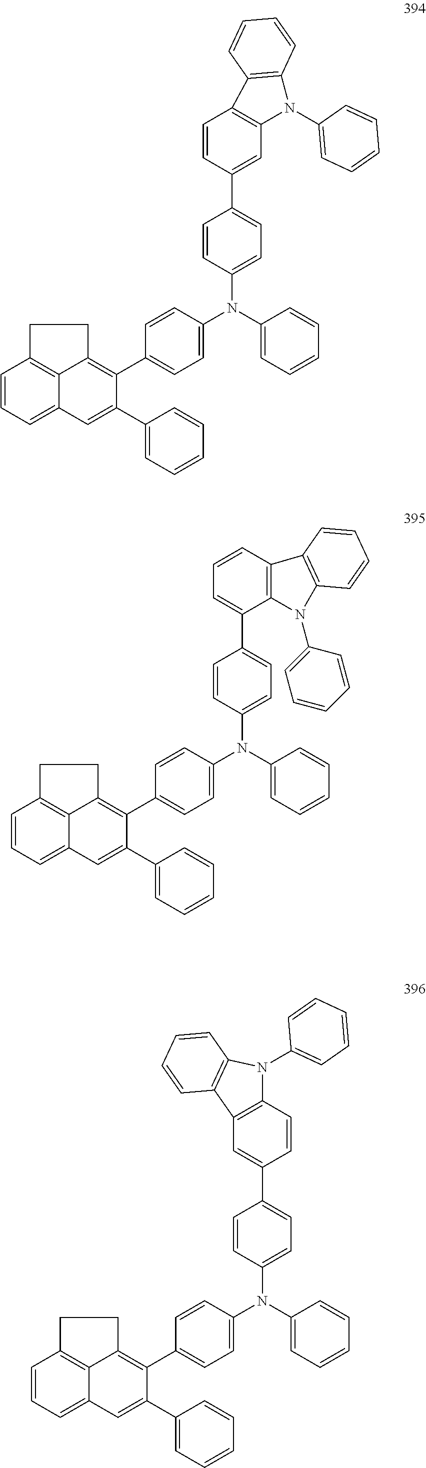 [SMILES: c1ccc(-c2cc3cccc4c3c(c2-c2ccc(N(c3ccccc3)c3ccc(-c5ccc6c7ccccc7n(-c7ccccc7)c6c5)cc3)cc2)CC4)cc1 ; c1ccc(-c2cc3cccc4c3c(c2-c2ccc(N(c3ccccc3)c3ccc(-c5cccc6c7ccccc7n(-c7ccccc7)c56)cc3)cc2)CC4)cc1 ; c1ccc(-c2cc3cccc4c3c(c2-c2ccc(N(c3ccccc3)c3ccc(-c5ccc6c(c5)c5ccccc5n6-c5ccccc5)cc3)cc2)CC4)cc1]

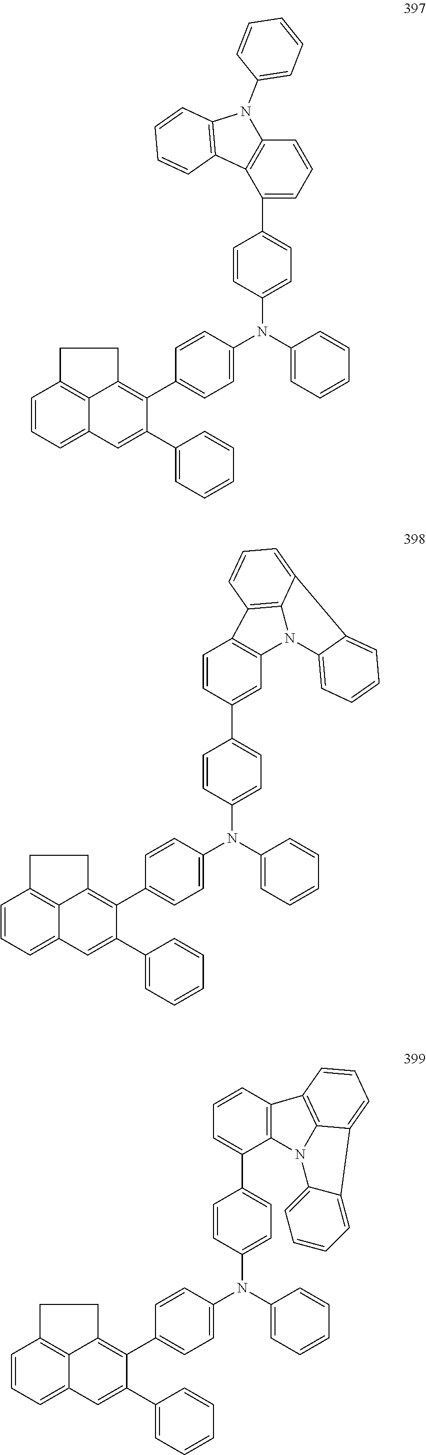 [SMILES: c1ccc(-c2cc3cccc4c3c(c2-c2ccc(N(c3ccccc3)c3ccc(-c5cccc6c5c5ccccc5n6-c5ccccc5)cc3)cc2)CC4)cc1 ; c1ccc(-c2cc3cccc4c3c(c2-c2ccc(N(c3ccccc3)c3ccc(-c5ccc6c7cccc8c9ccccc9n(c6c5)c87)cc3)cc2)CC4)cc1 ; c1ccc(-c2cc3cccc4c3c(c2-c2ccc(N(c3ccccc3)c3ccc(-c5cccc6c7cccc8c9ccccc9n(c56)c87)cc3)cc2)CC4)cc1]

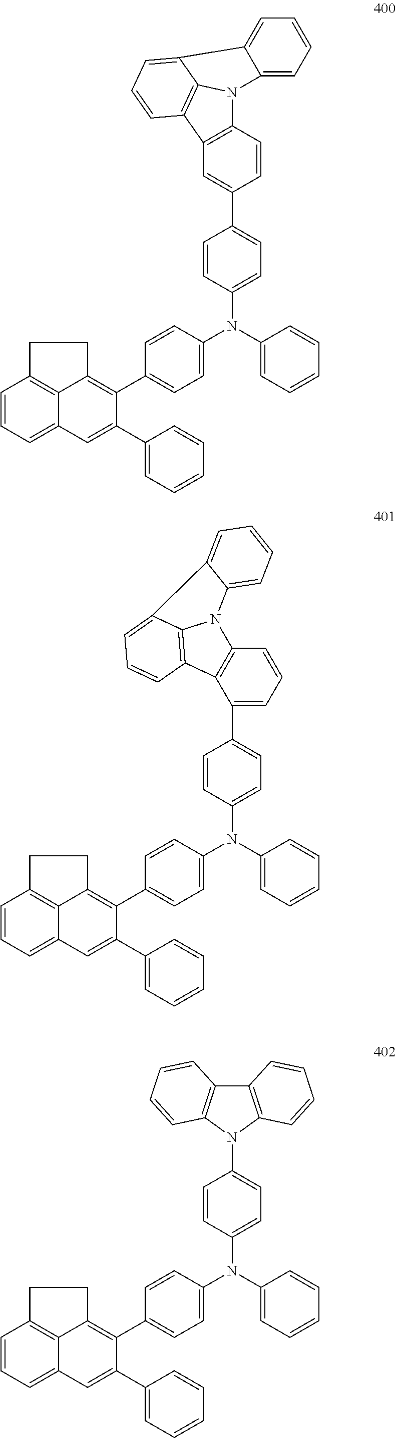 [SMILES: c1ccc(-c2cc3cccc4c3c(c2-c2ccc(N(c3ccccc3)c3ccc(-c5ccc6c(c5)c5cccc7c8ccccc8n6c75)cc3)cc2)CC4)cc1 ; c1ccc(-c2cc3cccc4c3c(c2-c2ccc(N(c3ccccc3)c3ccc(-c5cccc6c5c5cccc7c8ccccc8n6c75)cc3)cc2)CC4)cc1 ; c1ccc(-c2cc3cccc4c3c(c2-c2ccc(N(c3ccccc3)c3ccc(-n5c6ccccc6c6ccccc65)cc3)cc2)CC4)cc1]

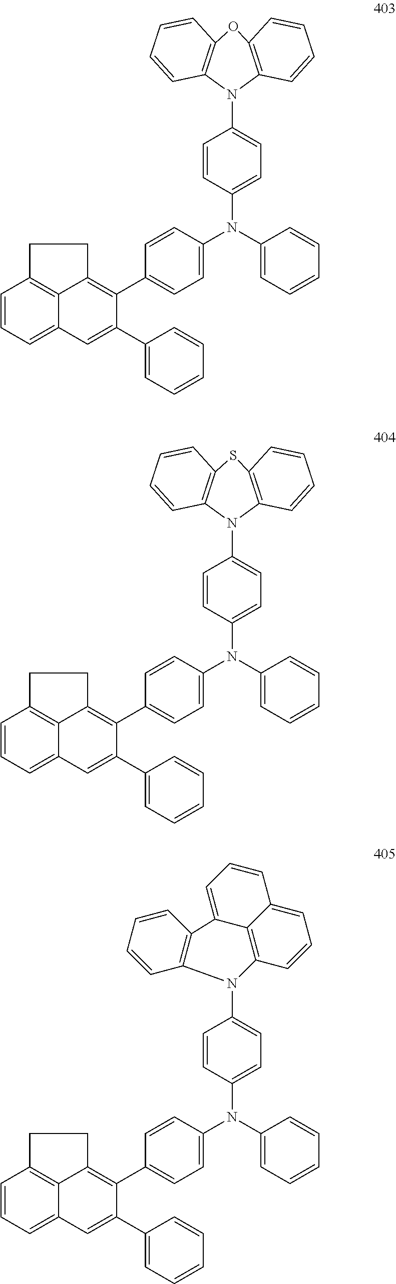 [SMILES: c1ccc(-c2cc3cccc4c3c(c2-c2ccc(N(c3ccccc3)c3ccc(N5c6ccccc6Oc6ccccc65)cc3)cc2)CC4)cc1 ; c1ccc(-c2cc3cccc4c3c(c2-c2ccc(N(c3ccccc3)c3ccc(N5c6ccccc6Sc6ccccc65)cc3)cc2)CC4)cc1 ; c1ccc(-c2cc3cccc4c3c(c2-c2ccc(N(c3ccccc3)c3ccc(N5c6ccccc6-c6cccc7cccc5c67)cc3)cc2)CC4)cc1]

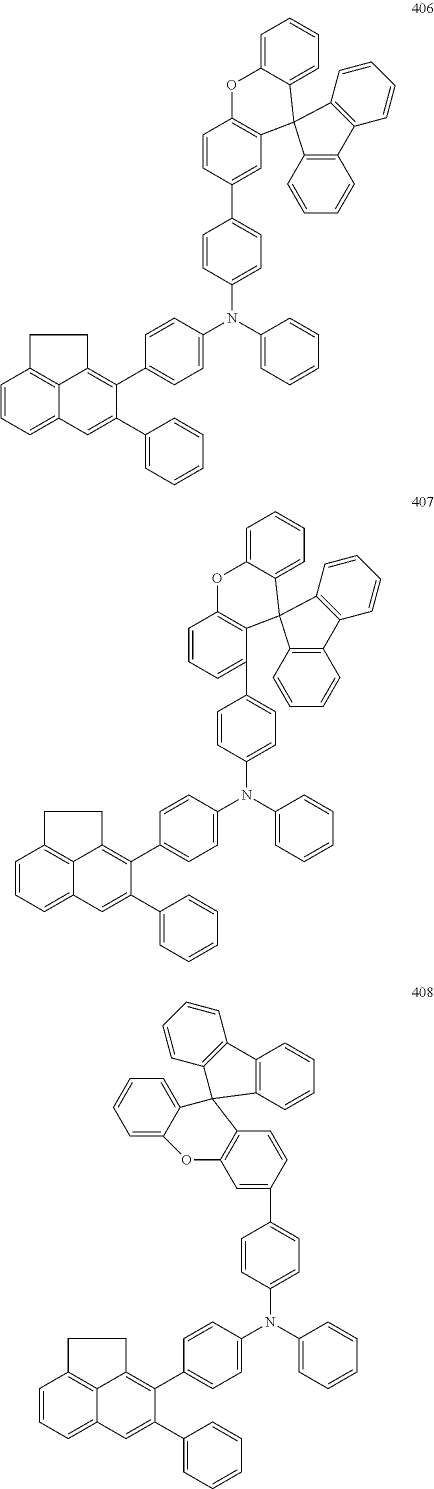

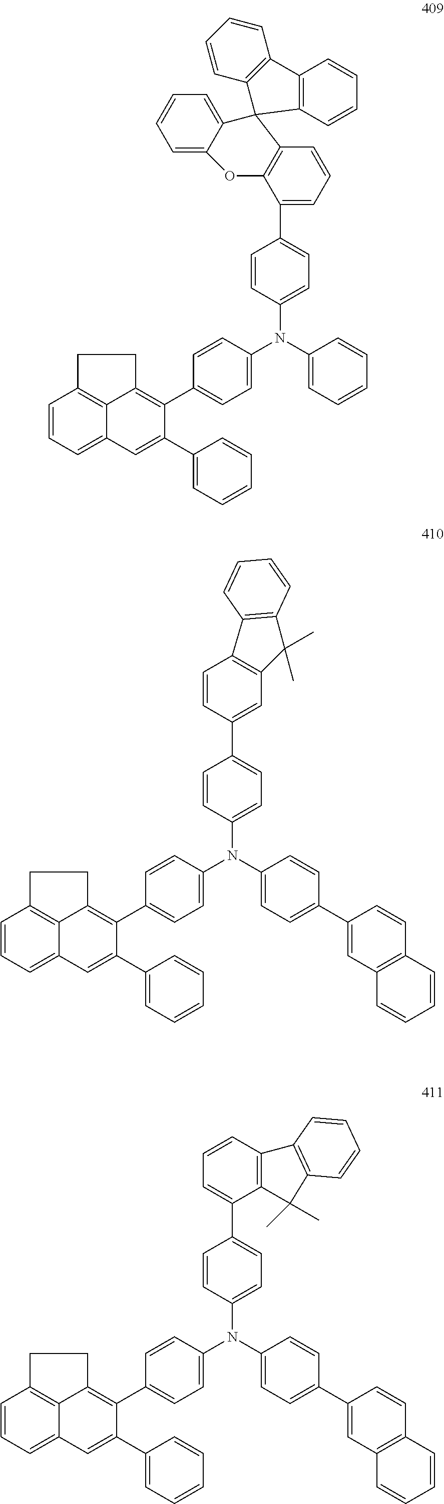 [SMILES: c1ccc(-c2cc3cccc4c3c(c2-c2ccc(N(c3ccccc3)c3ccc(-c5cccc6c5Oc5ccccc5C65c6ccccc6-c6ccccc65)cc3)cc2)CC4)cc1 ; CC1(C)c2ccccc2-c2ccc(-c3ccc(N(c4ccc(-c5ccc6ccccc6c5)cc4)c4ccc(-c5c(-c6ccccc6)cc6cccc7c6c5CC7)cc4)cc3)cc21 ; CC1(C)c2ccccc2-c2cccc(-c3ccc(N(c4ccc(-c5ccc6ccccc6c5)cc4)c4ccc(-c5c(-c6ccccc6)cc6cccc7c6c5CC7)cc4)cc3)c21]

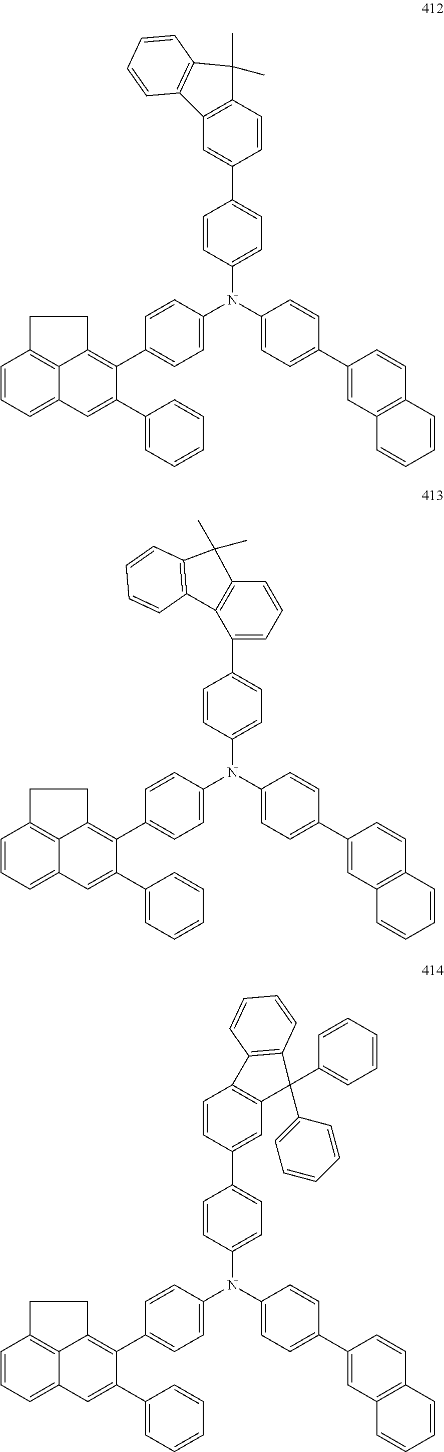 [SMILES: CC1(C)c2ccccc2-c2cc(-c3ccc(N(c4ccc(-c5ccc6ccccc6c5)cc4)c4ccc(-c5c(-c6ccccc6)cc6cccc7c6c5CC7)cc4)cc3)ccc21 ; CC1(C)c2ccccc2-c2c(-c3ccc(N(c4ccc(-c5ccc6ccccc6c5)cc4)c4ccc(-c5c(-c6ccccc6)cc6cccc7c6c5CC7)cc4)cc3)cccc21 ; c1ccc(-c2cc3cccc4c3c(c2-c2ccc(N(c3ccc(-c5ccc6c(c5)C(c5ccccc5)(c5ccccc5)c5ccccc5-6)cc3)c3ccc(-c5ccc6ccccc6c5)cc3)cc2)CC4)cc1]

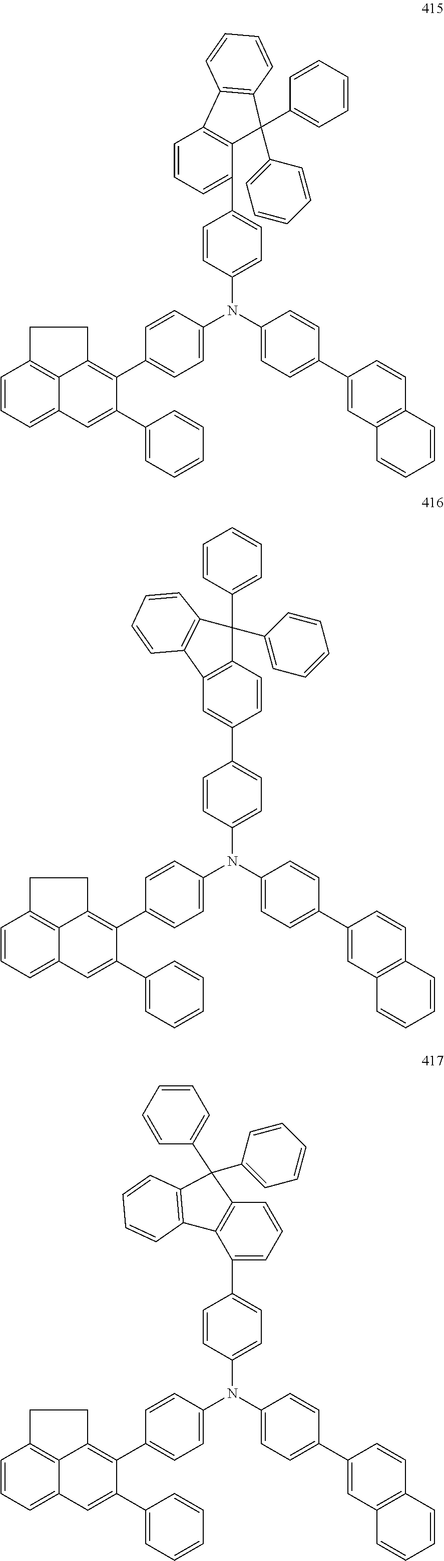

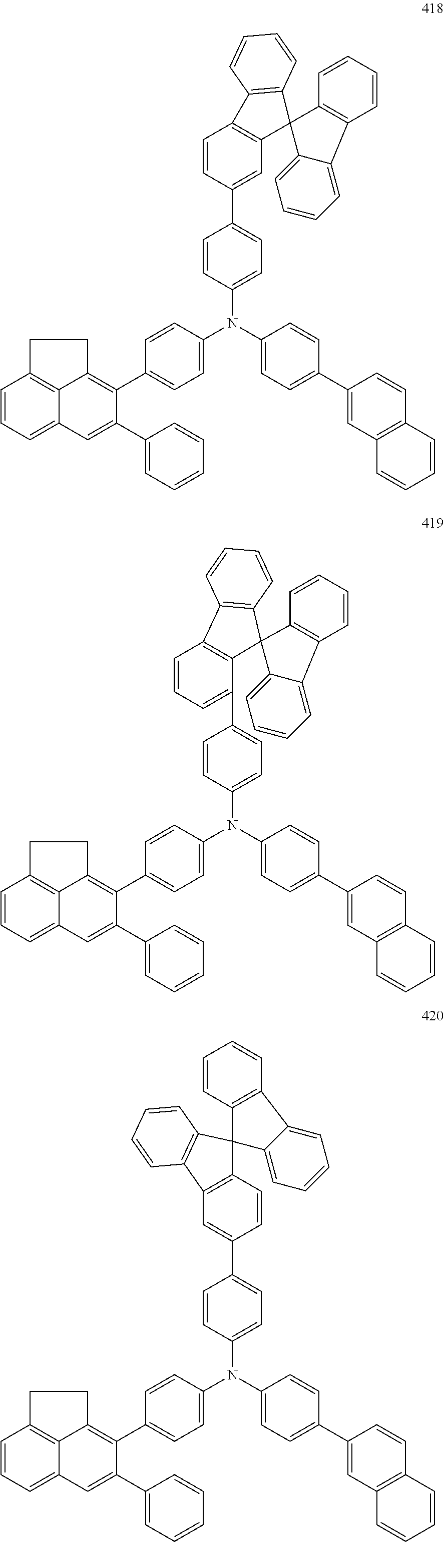 [SMILES: c1ccc(-c2cc3cccc4c3c(c2-c2ccc(N(c3ccc(-c5ccc6c(c5)C5(c7ccccc7-c7ccccc75)c5ccccc5-6)cc3)c3ccc(-c5ccc6ccccc6c5)cc3)cc2)CC4)cc1 ; c1ccc(-c2cc3cccc4c3c(c2-c2ccc(N(c3ccc(-c5ccc6ccccc6c5)cc3)c3ccc(-c5cccc6c5C5(c7ccccc7-c7ccccc75)c5ccccc5-6)cc3)cc2)CC4)cc1 ; c1ccc(-c2cc3cccc4c3c(c2-c2ccc(N(c3ccc(-c5ccc6c(c5)-c5ccccc5C65c6ccccc6-c6ccccc65)cc3)c3ccc(-c5ccc6ccccc6c5)cc3)cc2)CC4)cc1]

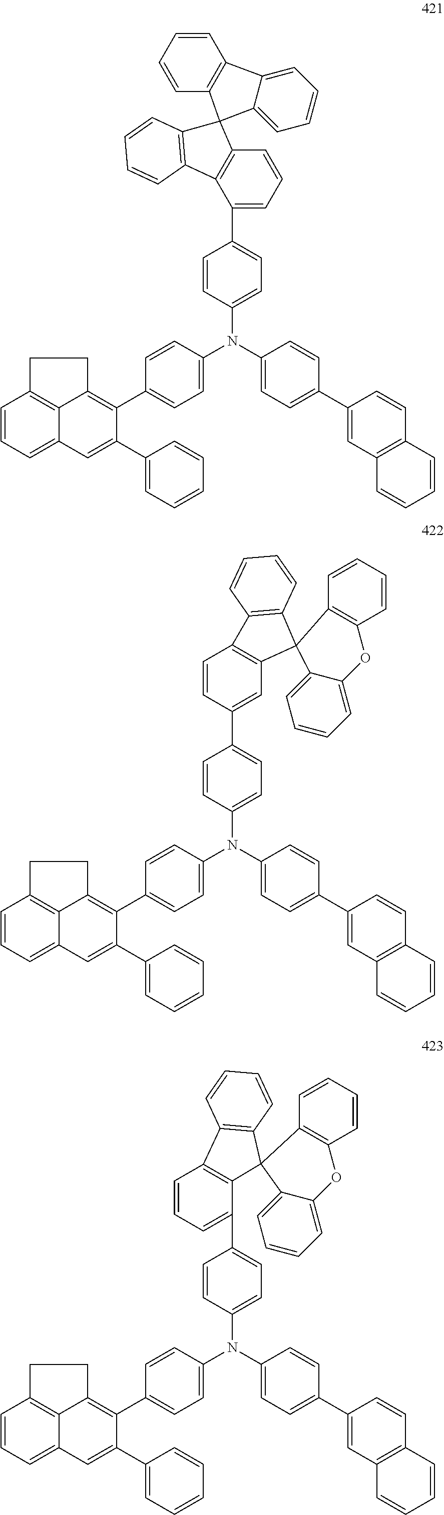 [SMILES: c1ccc(-c2cc3cccc4c3c(c2-c2ccc(N(c3ccc(-c5ccc6ccccc6c5)cc3)c3ccc(-c5cccc6c5-c5ccccc5C65c6ccccc6-c6ccccc65)cc3)cc2)CC4)cc1 ; c1ccc(-c2cc3cccc4c3c(c2-c2ccc(N(c3ccc(-c5ccc6c(c5)C5(c7ccccc7Oc7ccccc75)c5ccccc5-6)cc3)c3ccc(-c5ccc6ccccc6c5)cc3)cc2)CC4)cc1 ; c1ccc(-c2cc3cccc4c3c(c2-c2ccc(N(c3ccc(-c5ccc6ccccc6c5)cc3)c3ccc(-c5cccc6c5C5(c7ccccc7Oc7ccccc75)c5ccccc5-6)cc3)cc2)CC4)cc1]

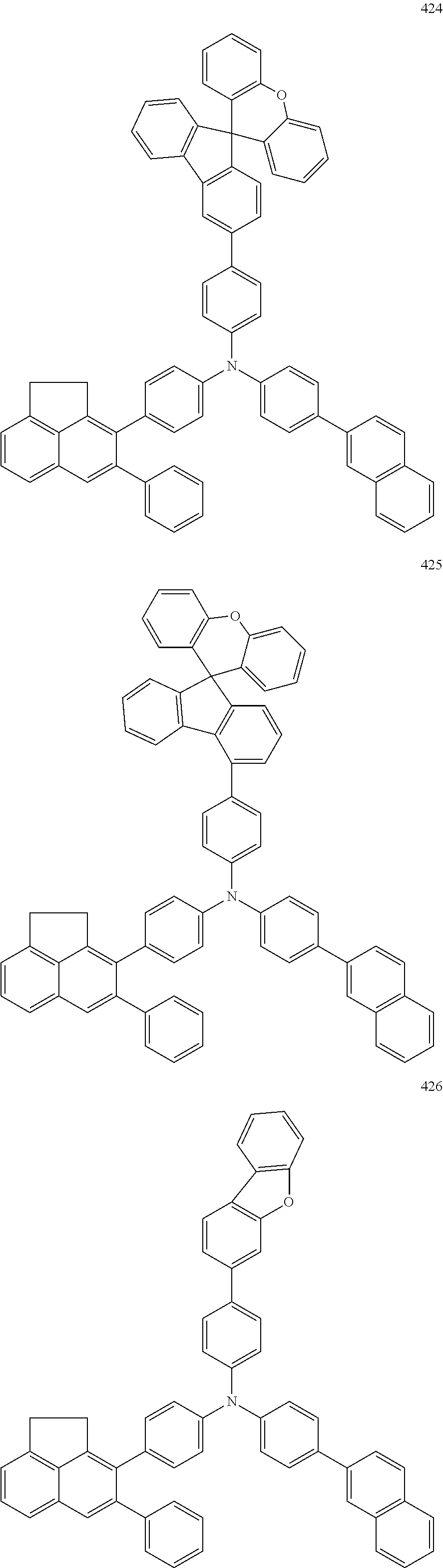

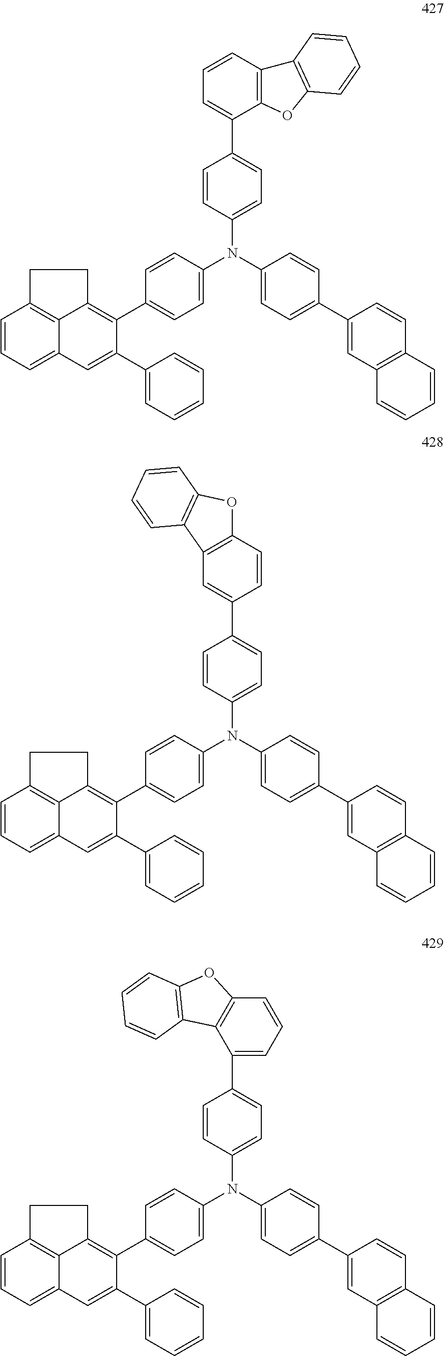 [SMILES: c1ccc(-c2cc3cccc4c3c(c2-c2ccc(N(c3ccc(-c5ccc6ccccc6c5)cc3)c3ccc(-c5cccc6c5oc5ccccc56)cc3)cc2)CC4)cc1 ; c1ccc(-c2cc3cccc4c3c(c2-c2ccc(N(c3ccc(-c5ccc6ccccc6c5)cc3)c3ccc(-c5ccc6oc7ccccc7c6c5)cc3)cc2)CC4)cc1 ; c1ccc(-c2cc3cccc4c3c(c2-c2ccc(N(c3ccc(-c5ccc6ccccc6c5)cc3)c3ccc(-c5cccc6oc7ccccc7c56)cc3)cc2)CC4)cc1]

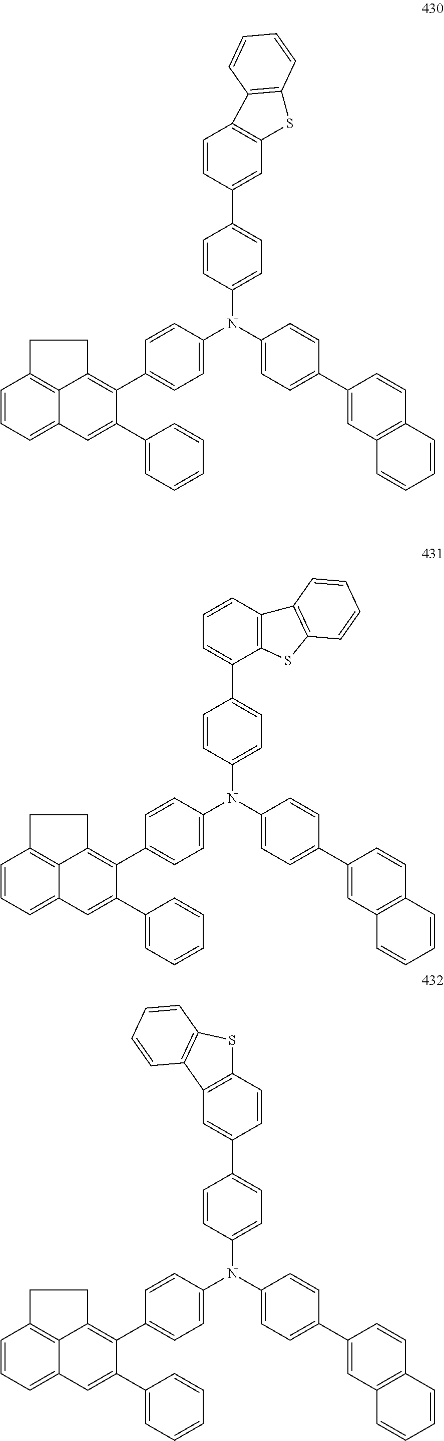

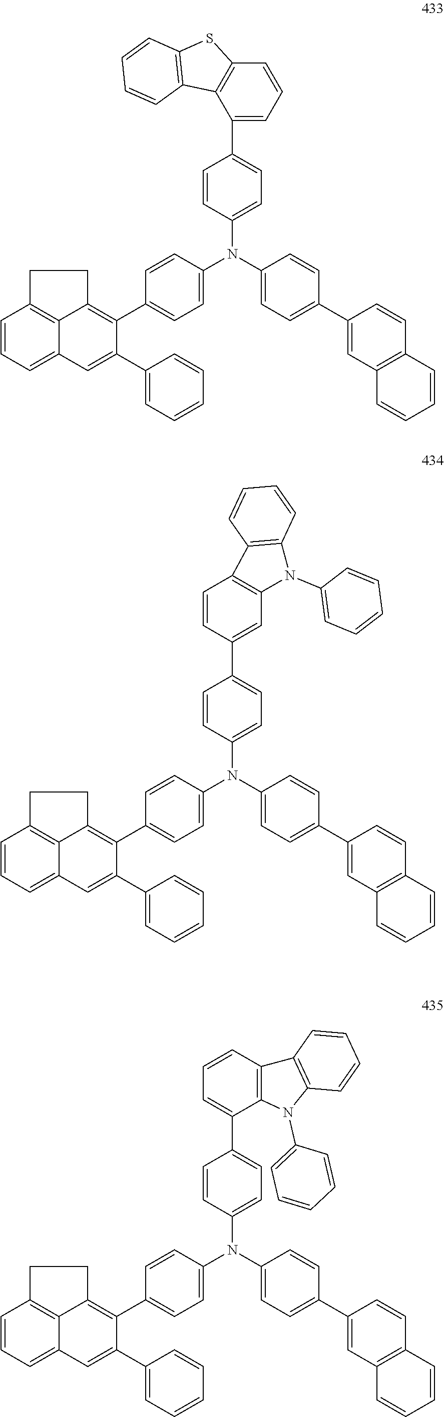 [SMILES: c1ccc(-c2cc3cccc4c3c(c2-c2ccc(N(c3ccc(-c5ccc6ccccc6c5)cc3)c3ccc(-c5cccc6sc7ccccc7c56)cc3)cc2)CC4)cc1 ; c1ccc(-c2cc3cccc4c3c(c2-c2ccc(N(c3ccc(-c5ccc6ccccc6c5)cc3)c3ccc(-c5ccc6c7ccccc7n(-c7ccccc7)c6c5)cc3)cc2)CC4)cc1 ; c1ccc(-c2cc3cccc4c3c(c2-c2ccc(N(c3ccc(-c5ccc6ccccc6c5)cc3)c3ccc(-c5cccc6c7ccccc7n(-c7ccccc7)c56)cc3)cc2)CC4)cc1]

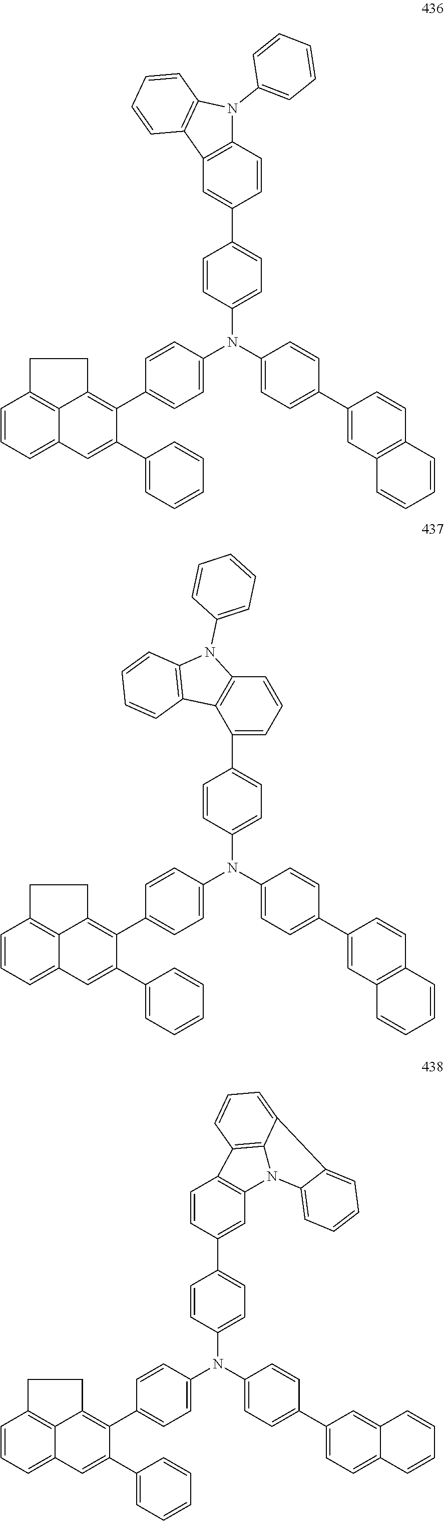 [SMILES: c1ccc(-c2cc3cccc4c3c(c2-c2ccc(N(c3ccc(-c5ccc6ccccc6c5)cc3)c3ccc(-c5ccc6c(c5)c5ccccc5n6-c5ccccc5)cc3)cc2)CC4)cc1 ; c1ccc(-c2cc3cccc4c3c(c2-c2ccc(N(c3ccc(-c5ccc6ccccc6c5)cc3)c3ccc(-c5cccc6c5c5ccccc5n6-c5ccccc5)cc3)cc2)CC4)cc1 ; c1ccc(-c2cc3cccc4c3c(c2-c2ccc(N(c3ccc(-c5ccc6ccccc6c5)cc3)c3ccc(-c5ccc6c7cccc8c9ccccc9n(c6c5)c87)cc3)cc2)CC4)cc1]

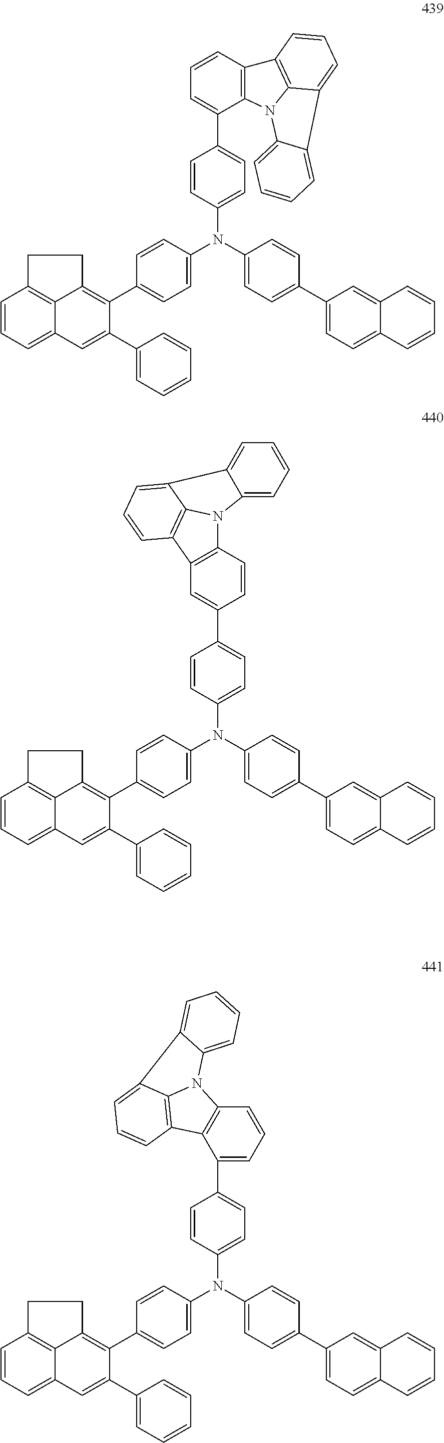 [SMILES: c1ccc(-c2cc3cccc4c3c(c2-c2ccc(N(c3ccc(-c5ccc6ccccc6c5)cc3)c3ccc(-c5cccc6c7cccc8c9ccccc9n(c56)c87)cc3)cc2)CC4)cc1 ; c1ccc(-c2cc3cccc4c3c(c2-c2ccc(N(c3ccc(-c5ccc6ccccc6c5)cc3)c3ccc(-c5ccc6c(c5)c5cccc7c8ccccc8n6c75)cc3)cc2)CC4)cc1 ; c1ccc(-c2cc3cccc4c3c(c2-c2ccc(N(c3ccc(-c5ccc6ccccc6c5)cc3)c3ccc(-c5cccc6c5c5cccc7c8ccccc8n6c75)cc3)cc2)CC4)cc1]

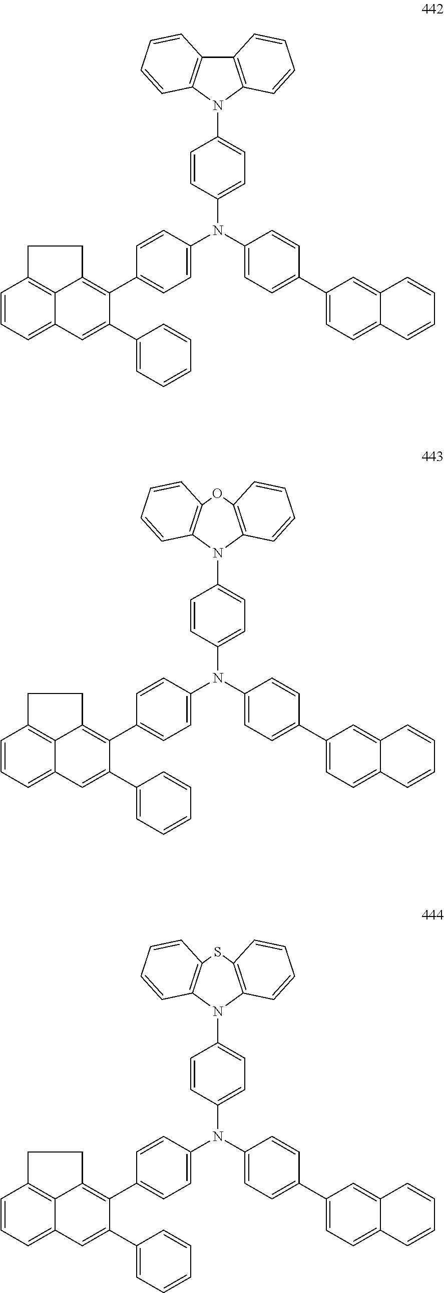 [SMILES: c1ccc(-c2cc3cccc4c3c(c2-c2ccc(N(c3ccc(-c5ccc6ccccc6c5)cc3)c3ccc(-n5c6ccccc6c6ccccc65)cc3)cc2)CC4)cc1 ; c1ccc(-c2cc3cccc4c3c(c2-c2ccc(N(c3ccc(-c5ccc6ccccc6c5)cc3)c3ccc(N5c6ccccc6Oc6ccccc65)cc3)cc2)CC4)cc1 ; c1ccc(-c2cc3cccc4c3c(c2-c2ccc(N(c3ccc(-c5ccc6ccccc6c5)cc3)c3ccc(N5c6ccccc6Sc6ccccc65)cc3)cc2)CC4)cc1]

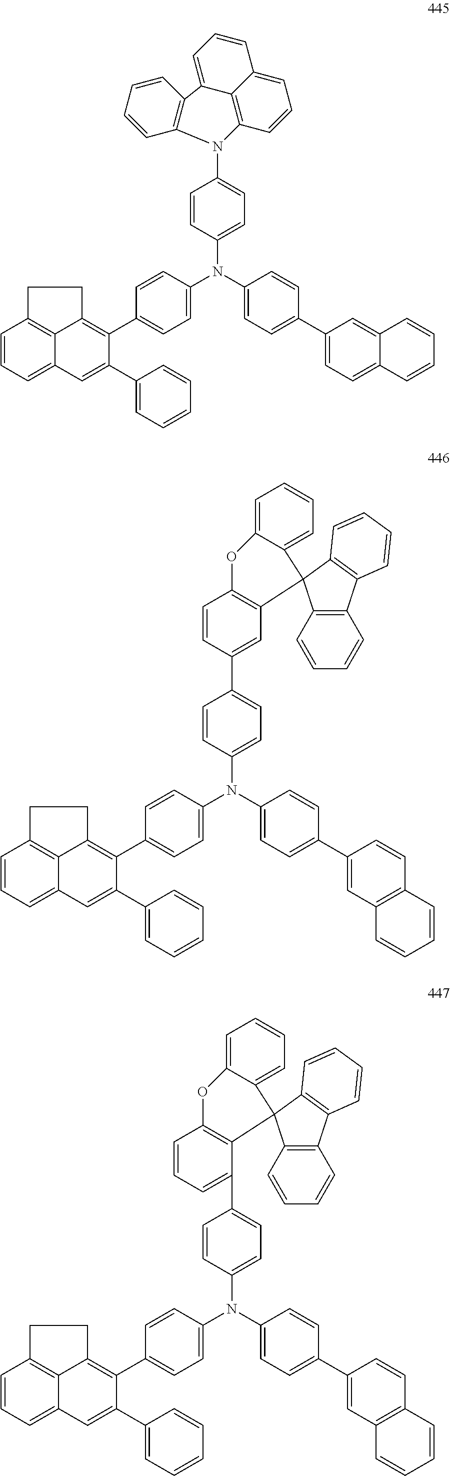 [SMILES: c1ccc(-c2cc3cccc4c3c(c2-c2ccc(N(c3ccc(-c5ccc6ccccc6c5)cc3)c3ccc(N5c6ccccc6-c6cccc7cccc5c67)cc3)cc2)CC4)cc1 ; c1ccc(-c2cc3cccc4c3c(c2-c2ccc(N(c3ccc(-c5ccc6c(c5)C5(c7ccccc7O6)c6ccccc6-c6ccccc65)cc3)c3ccc(-c5ccc6ccccc6c5)cc3)cc2)CC4)cc1 ; c1ccc(-c2cc3cccc4c3c(c2-c2ccc(N(c3ccc(-c5ccc6ccccc6c5)cc3)c3ccc(-c5cccc6c5C5(c7ccccc7O6)c6ccccc6-c6ccccc65)cc3)cc2)CC4)cc1]

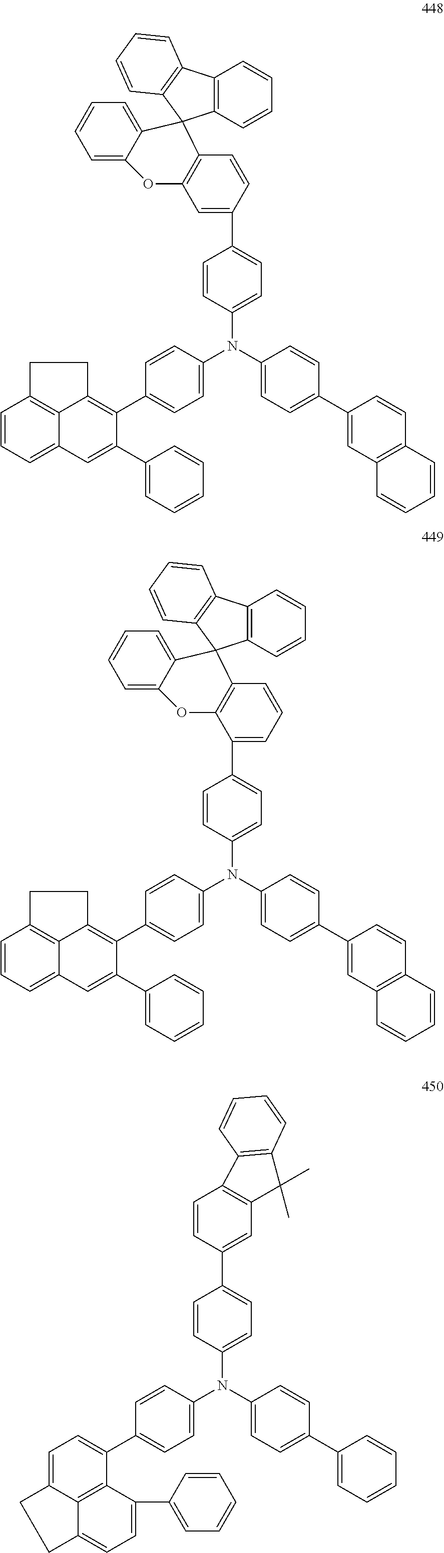 [SMILES: c1ccc(-c2cc3cccc4c3c(c2-c2ccc(N(c3ccc(-c5ccc6c(c5)Oc5ccccc5C65c6ccccc6-c6ccccc65)cc3)c3ccc(-c5ccc6ccccc6c5)cc3)cc2)CC4)cc1 ; c1ccc(-c2cc3cccc4c3c(c2-c2ccc(N(c3ccc(-c5ccc6ccccc6c5)cc3)c3ccc(-c5cccc6c5Oc5ccccc5C65c6ccccc6-c6ccccc65)cc3)cc2)CC4)cc1 ; CC1(C)c2ccccc2-c2ccc(-c3ccc(N(c4ccc(-c5ccccc5)cc4)c4ccc(-c5ccc6c7c(ccc(-c8ccccc8)c57)CC6)cc4)cc3)cc21]

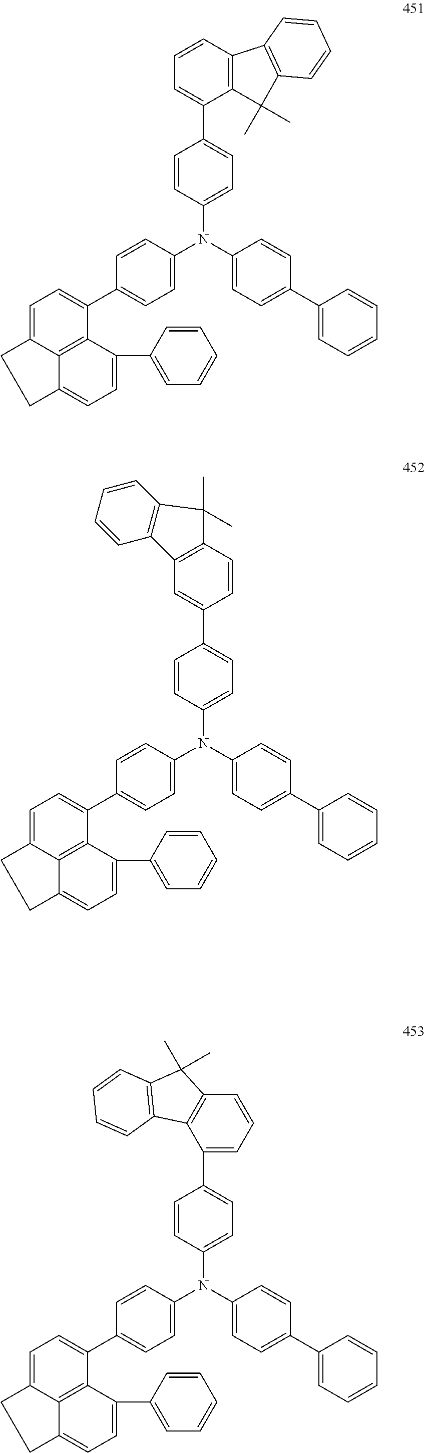 [SMILES: CC1(C)c2ccccc2-c2cccc(-c3ccc(N(c4ccc(-c5ccccc5)cc4)c4ccc(-c5ccc6c7c(ccc(-c8ccccc8)c57)CC6)cc4)cc3)c21 ; CC1(C)c2ccccc2-c2cc(-c3ccc(N(c4ccc(-c5ccccc5)cc4)c4ccc(-c5ccc6c7c(ccc(-c8ccccc8)c57)CC6)cc4)cc3)ccc21 ; CC1(C)c2ccccc2-c2c(-c3ccc(N(c4ccc(-c5ccccc5)cc4)c4ccc(-c5ccc6c7c(ccc(-c8ccccc8)c57)CC6)cc4)cc3)cccc21]

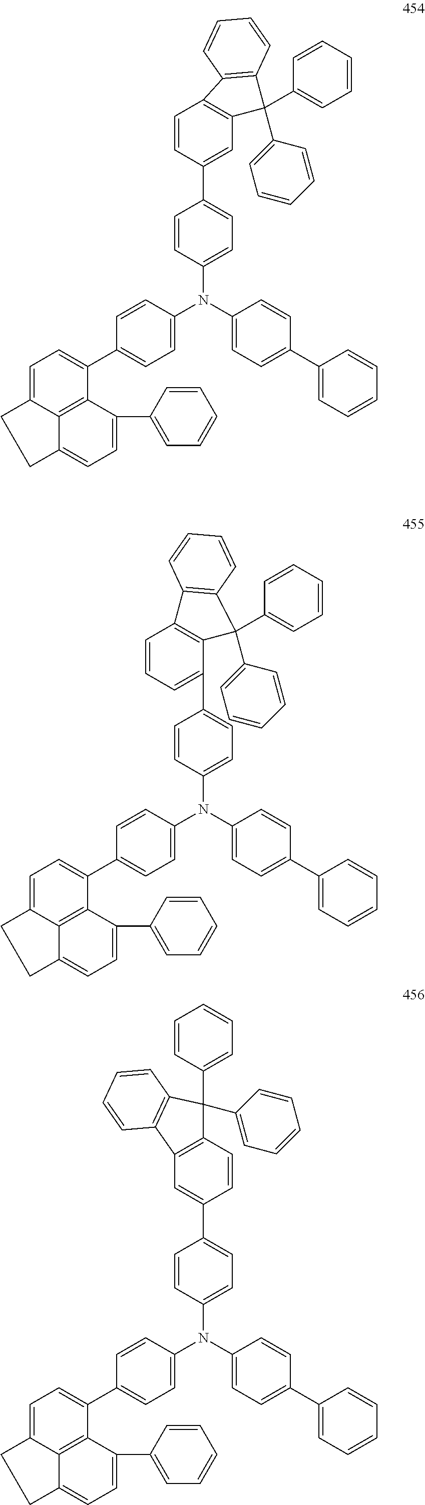 [SMILES: c1ccc(-c2ccc(N(c3ccc(-c4ccc5c(c4)C(c4ccccc4)(c4ccccc4)c4ccccc4-5)cc3)c3ccc(-c4ccc5c6c(ccc(-c7ccccc7)c46)CC5)cc3)cc2)cc1 ; c1ccc(-c2ccc(N(c3ccc(-c4cccc5c4C(c4ccccc4)(c4ccccc4)c4ccccc4-5)cc3)c3ccc(-c4ccc5c6c(ccc(-c7ccccc7)c46)CC5)cc3)cc2)cc1 ; c1ccc(-c2ccc(N(c3ccc(-c4ccc5c(c4)-c4ccccc4C5(c4ccccc4)c4ccccc4)cc3)c3ccc(-c4ccc5c6c(ccc(-c7ccccc7)c46)CC5)cc3)cc2)cc1]

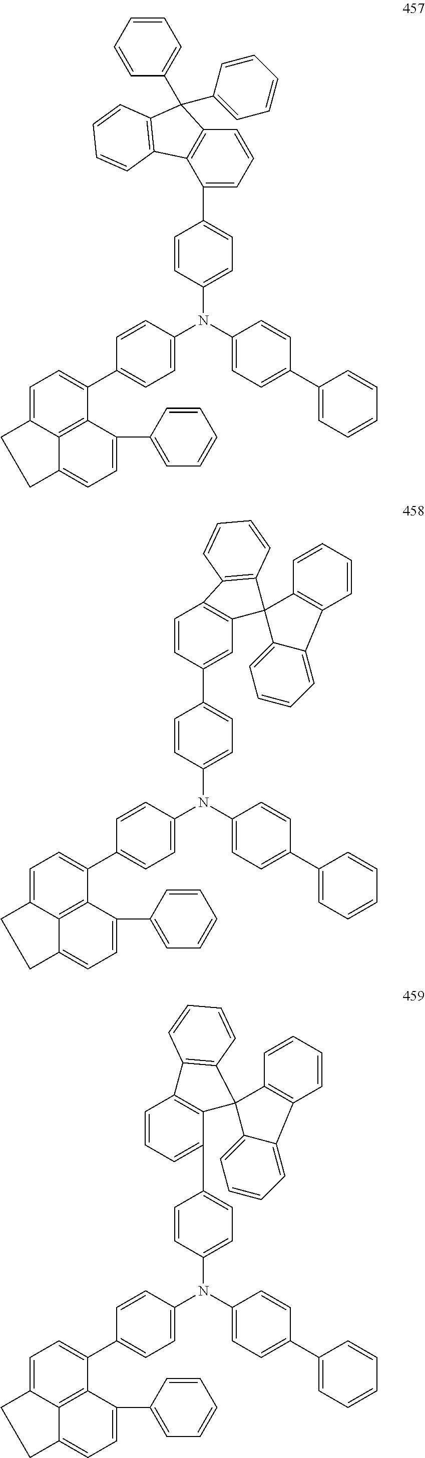 [SMILES: c1ccc(-c2ccc(N(c3ccc(-c4cccc5c4-c4ccccc4C5(c4ccccc4)c4ccccc4)cc3)c3ccc(-c4ccc5c6c(ccc(-c7ccccc7)c46)CC5)cc3)cc2)cc1 ; c1ccc(-c2ccc(N(c3ccc(-c4ccc5c(c4)C4(c6ccccc6-c6ccccc64)c4ccccc4-5)cc3)c3ccc(-c4ccc5c6c(ccc(-c7ccccc7)c46)CC5)cc3)cc2)cc1 ; c1ccc(-c2ccc(N(c3ccc(-c4cccc5c4C4(c6ccccc6-c6ccccc64)c4ccccc4-5)cc3)c3ccc(-c4ccc5c6c(ccc(-c7ccccc7)c46)CC5)cc3)cc2)cc1]

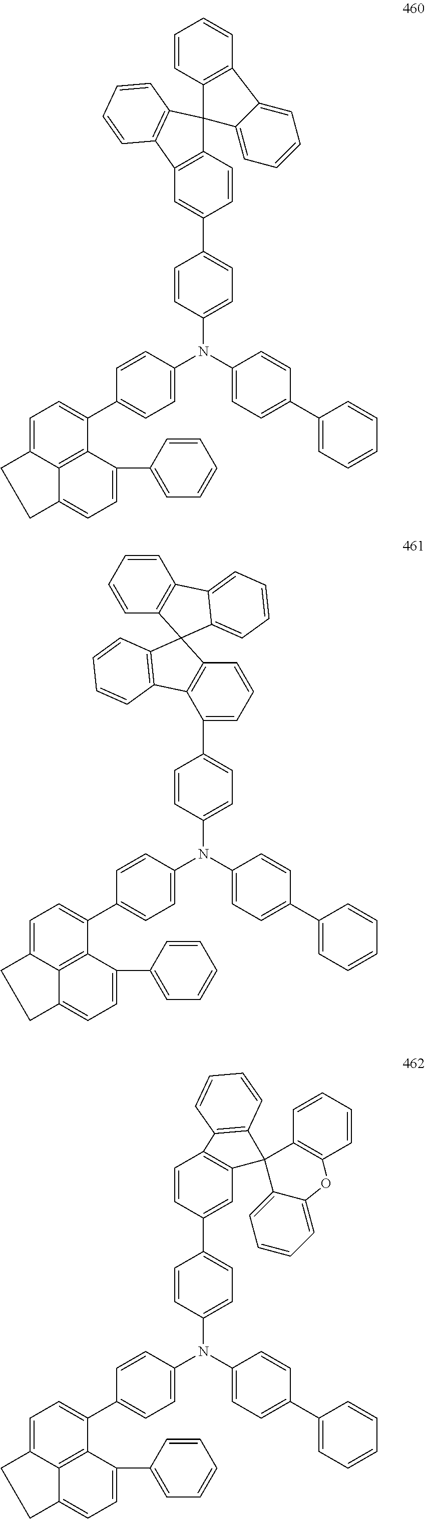 [SMILES: c1ccc(-c2ccc(N(c3ccc(-c4ccc5c(c4)-c4ccccc4C54c5ccccc5-c5ccccc54)cc3)c3ccc(-c4ccc5c6c(ccc(-c7ccccc7)c46)CC5)cc3)cc2)cc1 ; c1ccc(-c2ccc(N(c3ccc(-c4cccc5c4-c4ccccc4C54c5ccccc5-c5ccccc54)cc3)c3ccc(-c4ccc5c6c(ccc(-c7ccccc7)c46)CC5)cc3)cc2)cc1 ; c1ccc(-c2ccc(N(c3ccc(-c4ccc5c(c4)C4(c6ccccc6Oc6ccccc64)c4ccccc4-5)cc3)c3ccc(-c4ccc5c6c(ccc(-c7ccccc7)c46)CC5)cc3)cc2)cc1]

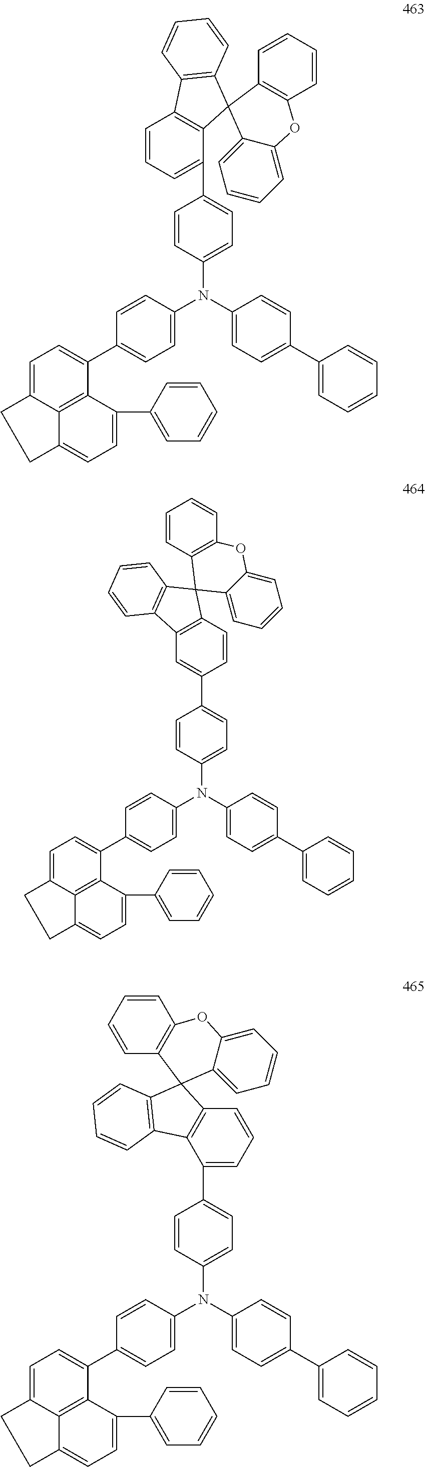 [SMILES: c1ccc(-c2ccc(N(c3ccc(-c4cccc5c4C4(c6ccccc6Oc6ccccc64)c4ccccc4-5)cc3)c3ccc(-c4ccc5c6c(ccc(-c7ccccc7)c46)CC5)cc3)cc2)cc1 ; c1ccc(-c2ccc(N(c3ccc(-c4ccc5c(c4)-c4ccccc4C54c5ccccc5Oc5ccccc54)cc3)c3ccc(-c4ccc5c6c(ccc(-c7ccccc7)c46)CC5)cc3)cc2)cc1 ; c1ccc(-c2ccc(N(c3ccc(-c4cccc5c4-c4ccccc4C54c5ccccc5Oc5ccccc54)cc3)c3ccc(-c4ccc5c6c(ccc(-c7ccccc7)c46)CC5)cc3)cc2)cc1]

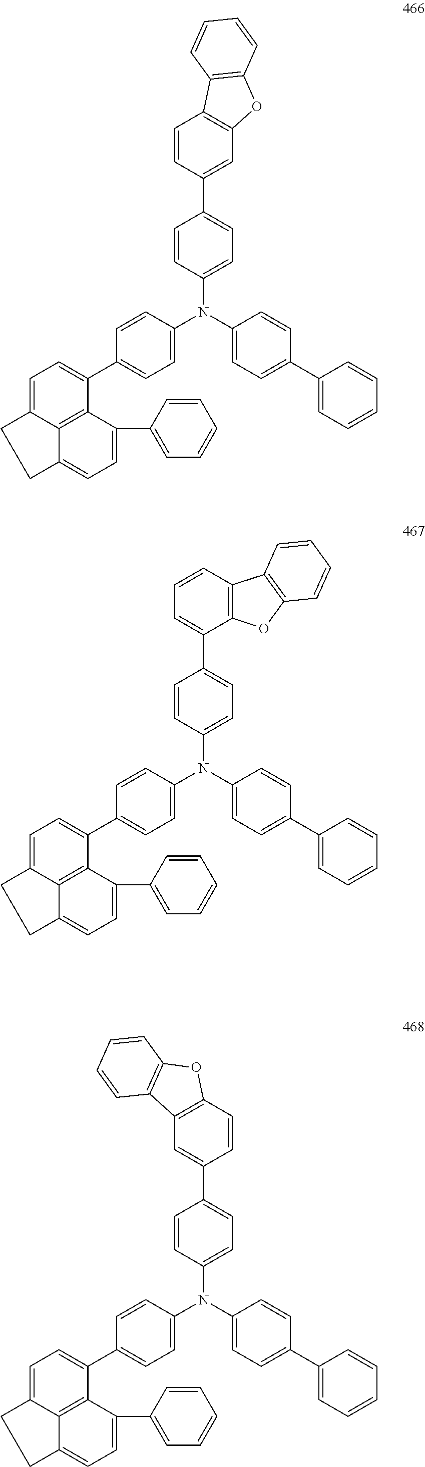 [SMILES: c1ccc(-c2ccc(N(c3ccc(-c4ccc5c(c4)oc4ccccc45)cc3)c3ccc(-c4ccc5c6c(ccc(-c7ccccc7)c46)CC5)cc3)cc2)cc1 ; c1ccc(-c2ccc(N(c3ccc(-c4cccc5c4oc4ccccc45)cc3)c3ccc(-c4ccc5c6c(ccc(-c7ccccc7)c46)CC5)cc3)cc2)cc1 ; c1ccc(-c2ccc(N(c3ccc(-c4ccc5oc6ccccc6c5c4)cc3)c3ccc(-c4ccc5c6c(ccc(-c7ccccc7)c46)CC5)cc3)cc2)cc1]

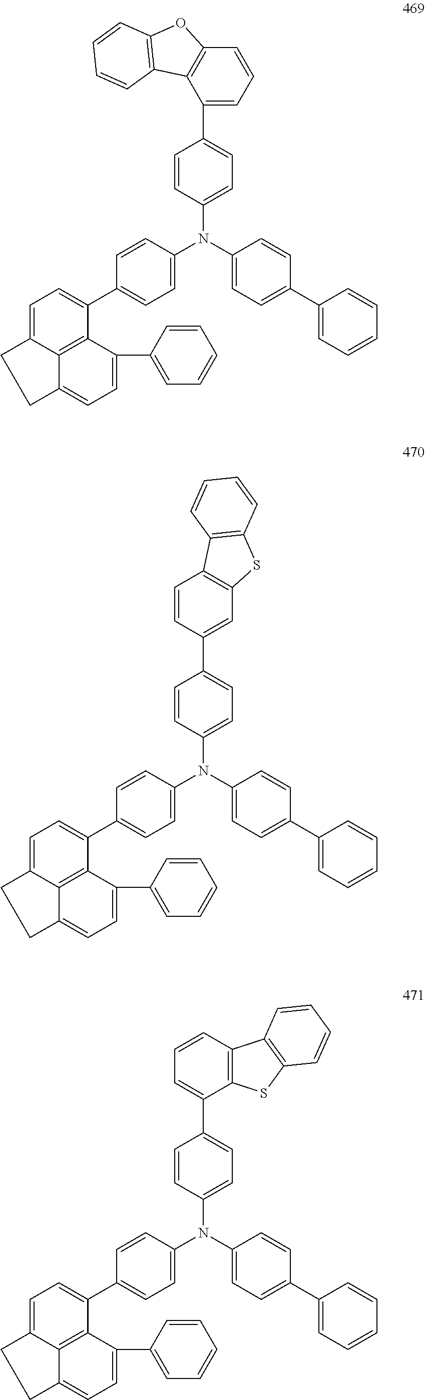 [SMILES: c1ccc(-c2ccc(N(c3ccc(-c4ccc5c6c(ccc(-c7ccccc7)c46)CC5)cc3)c3ccc(-c4cccc5oc6ccccc6c45)cc3)cc2)cc1 ; c1ccc(-c2ccc(N(c3ccc(-c4ccc5c(c4)sc4ccccc45)cc3)c3ccc(-c4ccc5c6c(ccc(-c7ccccc7)c46)CC5)cc3)cc2)cc1 ; c1ccc(-c2ccc(N(c3ccc(-c4cccc5c4sc4ccccc45)cc3)c3ccc(-c4ccc5c6c(ccc(-c7ccccc7)c46)CC5)cc3)cc2)cc1]

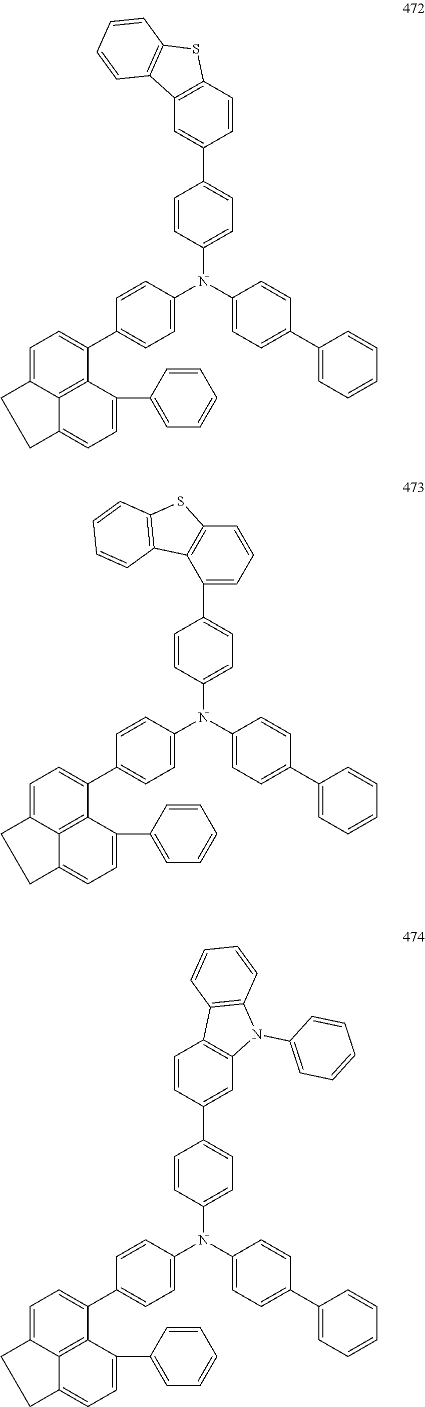 [SMILES: c1ccc(-c2ccc(N(c3ccc(-c4ccc5sc6ccccc6c5c4)cc3)c3ccc(-c4ccc5c6c(ccc(-c7ccccc7)c46)CC5)cc3)cc2)cc1 ; c1ccc(-c2ccc(N(c3ccc(-c4ccc5c6c(ccc(-c7ccccc7)c46)CC5)cc3)c3ccc(-c4cccc5sc6ccccc6c45)cc3)cc2)cc1 ; c1ccc(-c2ccc(N(c3ccc(-c4ccc5c6ccccc6n(-c6ccccc6)c5c4)cc3)c3ccc(-c4ccc5c6c(ccc(-c7ccccc7)c46)CC5)cc3)cc2)cc1]

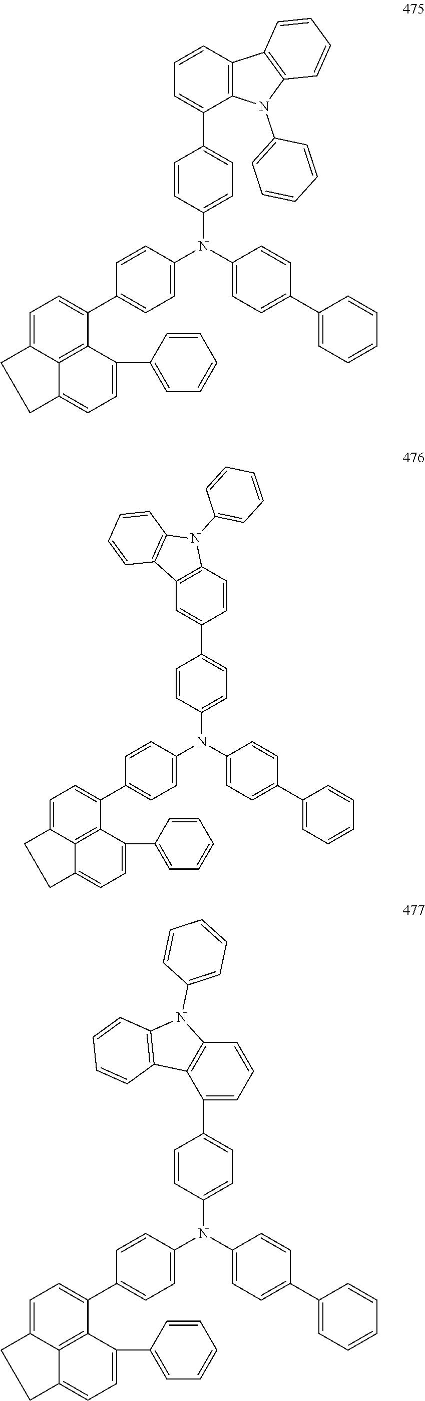 [SMILES: c1ccc(-c2ccc(N(c3ccc(-c4ccc5c6c(ccc(-c7ccccc7)c46)CC5)cc3)c3ccc(-c4cccc5c6ccccc6n(-c6ccccc6)c45)cc3)cc2)cc1 ; c1ccc(-c2ccc(N(c3ccc(-c4ccc5c(c4)c4ccccc4n5-c4ccccc4)cc3)c3ccc(-c4ccc5c6c(ccc(-c7ccccc7)c46)CC5)cc3)cc2)cc1 ; c1ccc(-c2ccc(N(c3ccc(-c4ccc5c6c(ccc(-c7ccccc7)c46)CC5)cc3)c3ccc(-c4cccc5c4c4ccccc4n5-c4ccccc4)cc3)cc2)cc1]

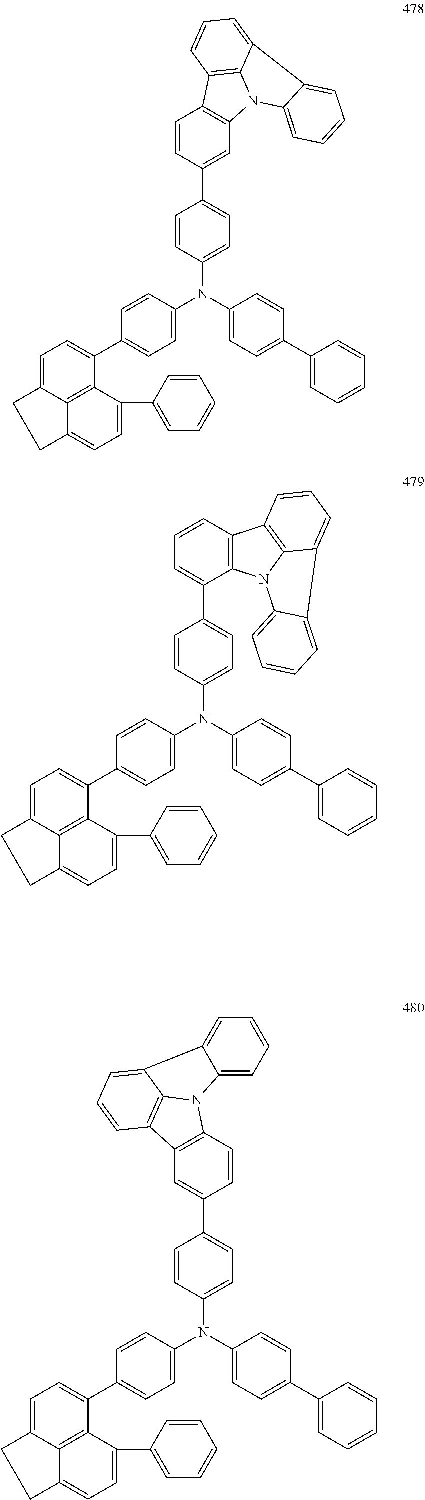 [SMILES: c1ccc(-c2ccc(N(c3ccc(-c4ccc5c6cccc7c8ccccc8n(c5c4)c76)cc3)c3ccc(-c4ccc5c6c(ccc(-c7ccccc7)c46)CC5)cc3)cc2)cc1 ; c1ccc(-c2ccc(N(c3ccc(-c4ccc5c6c(ccc(-c7ccccc7)c46)CC5)cc3)c3ccc(-c4cccc5c6cccc7c8ccccc8n(c45)c76)cc3)cc2)cc1 ; c1ccc(-c2ccc(N(c3ccc(-c4ccc5c(c4)c4cccc6c7ccccc7n5c64)cc3)c3ccc(-c4ccc5c6c(ccc(-c7ccccc7)c46)CC5)cc3)cc2)cc1]

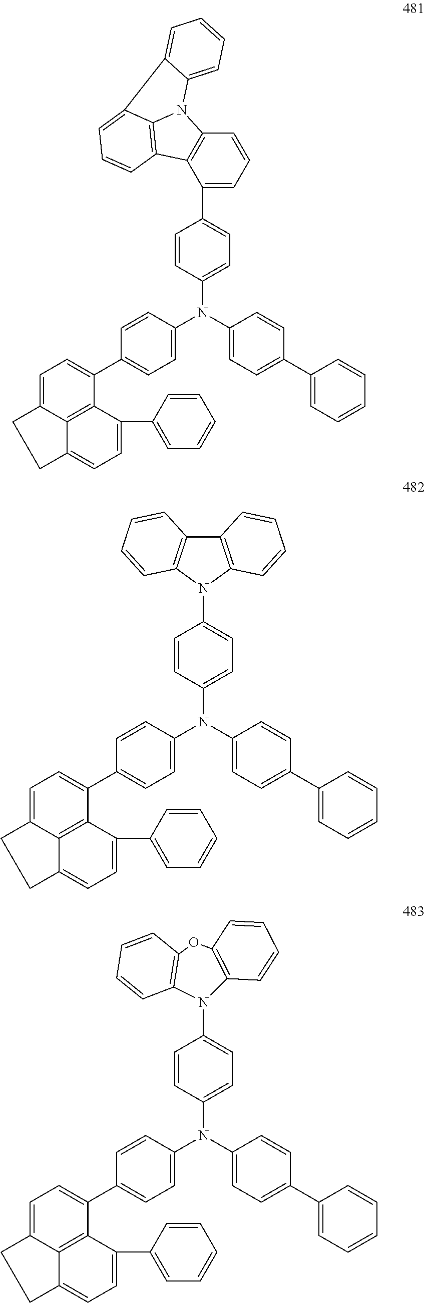 [SMILES: c1ccc(-c2ccc(N(c3ccc(-c4ccc5c6c(ccc(-c7ccccc7)c46)CC5)cc3)c3ccc(-c4cccc5c4c4cccc6c7ccccc7n5c64)cc3)cc2)cc1 ; c1ccc(-c2ccc(N(c3ccc(-c4ccc5c6c(ccc(-c7ccccc7)c46)CC5)cc3)c3ccc(-n4c5ccccc5c5ccccc54)cc3)cc2)cc1 ; c1ccc(-c2ccc(N(c3ccc(-c4ccc5c6c(ccc(-c7ccccc7)c46)CC5)cc3)c3ccc(N4c5ccccc5Oc5ccccc54)cc3)cc2)cc1]

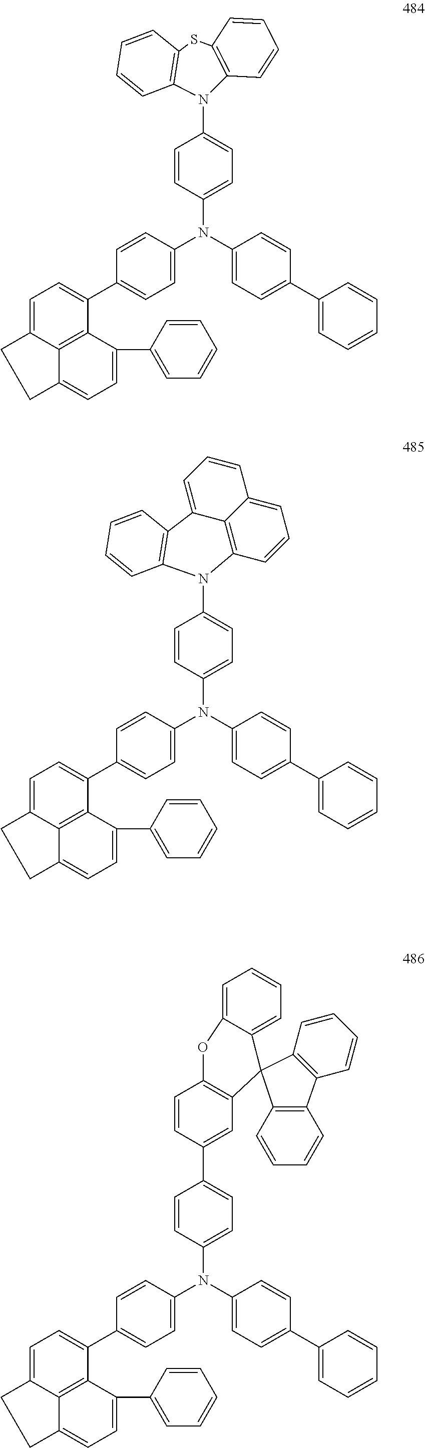 [SMILES: c1ccc(-c2ccc(N(c3ccc(-c4ccc5c6c(ccc(-c7ccccc7)c46)CC5)cc3)c3ccc(N4c5ccccc5Sc5ccccc54)cc3)cc2)cc1 ; c1ccc(-c2ccc(N(c3ccc(-c4ccc5c6c(ccc(-c7ccccc7)c46)CC5)cc3)c3ccc(N4c5ccccc5-c5cccc6cccc4c56)cc3)cc2)cc1 ; c1ccc(-c2ccc(N(c3ccc(-c4ccc5c(c4)C4(c6ccccc6O5)c5ccccc5-c5ccccc54)cc3)c3ccc(-c4ccc5c6c(ccc(-c7ccccc7)c46)CC5)cc3)cc2)cc1]

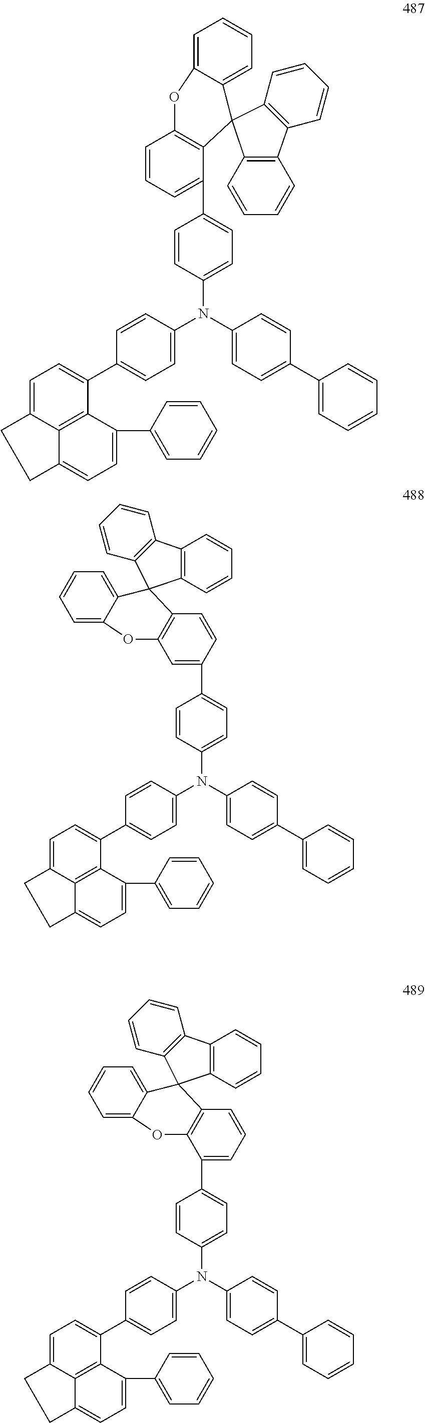

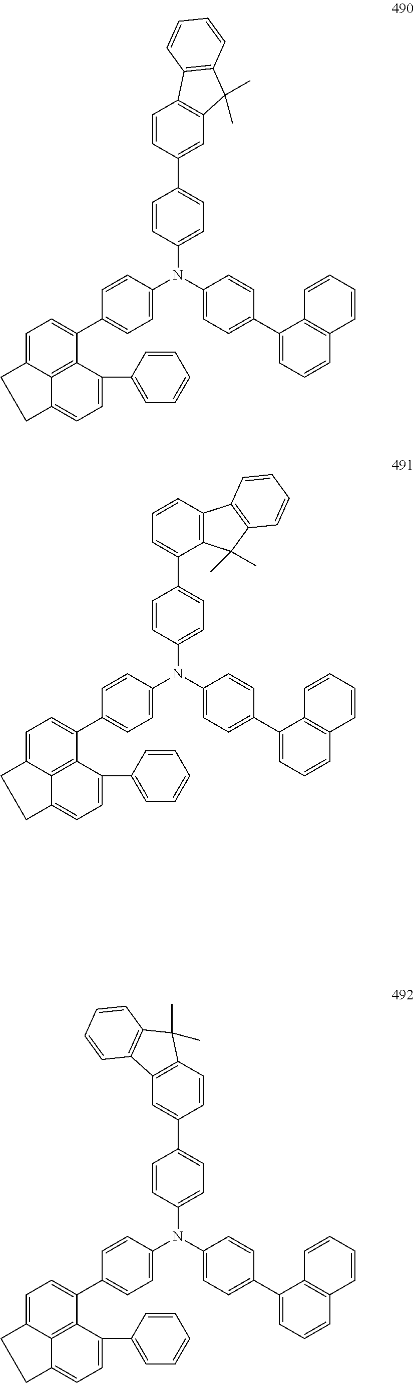 [SMILES: CC1(C)c2ccccc2-c2ccc(-c3ccc(N(c4ccc(-c5cccc6ccccc56)cc4)c4ccc(-c5ccc6c7c(ccc(-c8ccccc8)c57)CC6)cc4)cc3)cc21 ; CC1(C)c2ccccc2-c2cccc(-c3ccc(N(c4ccc(-c5cccc6ccccc56)cc4)c4ccc(-c5ccc6c7c(ccc(-c8ccccc8)c57)CC6)cc4)cc3)c21 ; CC1(C)c2ccccc2-c2cc(-c3ccc(N(c4ccc(-c5cccc6ccccc56)cc4)c4ccc(-c5ccc6c7c(ccc(-c8ccccc8)c57)CC6)cc4)cc3)ccc21]

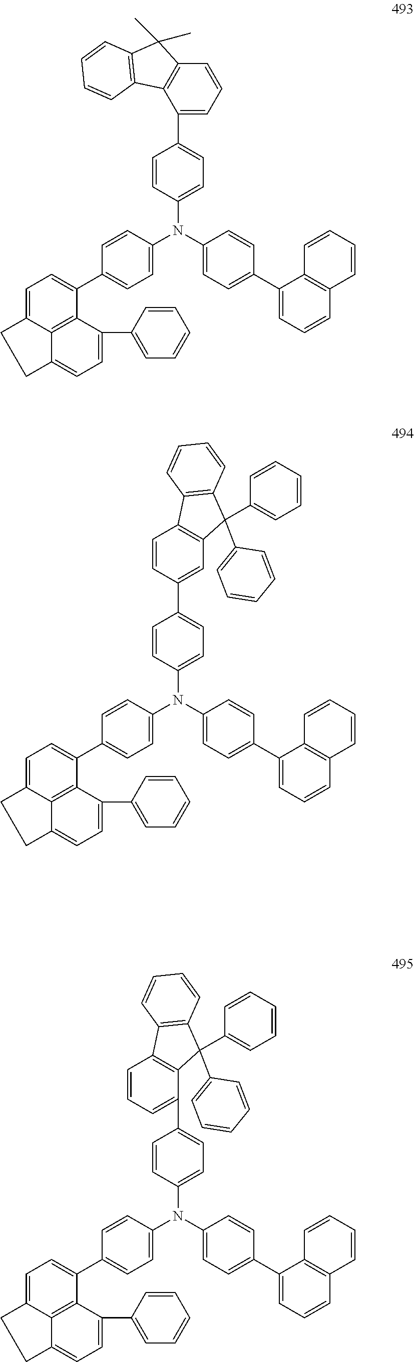 [SMILES: CC1(C)c2ccccc2-c2c(-c3ccc(N(c4ccc(-c5cccc6ccccc56)cc4)c4ccc(-c5ccc6c7c(ccc(-c8ccccc8)c57)CC6)cc4)cc3)cccc21 ; c1ccc(-c2ccc3c4c(ccc(-c5ccc(N(c6ccc(-c7ccc8c(c7)C(c7ccccc7)(c7ccccc7)c7ccccc7-8)cc6)c6ccc(-c7cccc8ccccc78)cc6)cc5)c24)CC3)cc1 ; c1ccc(-c2ccc3c4c(ccc(-c5ccc(N(c6ccc(-c7cccc8c7C(c7ccccc7)(c7ccccc7)c7ccccc7-8)cc6)c6ccc(-c7cccc8ccccc78)cc6)cc5)c24)CC3)cc1]

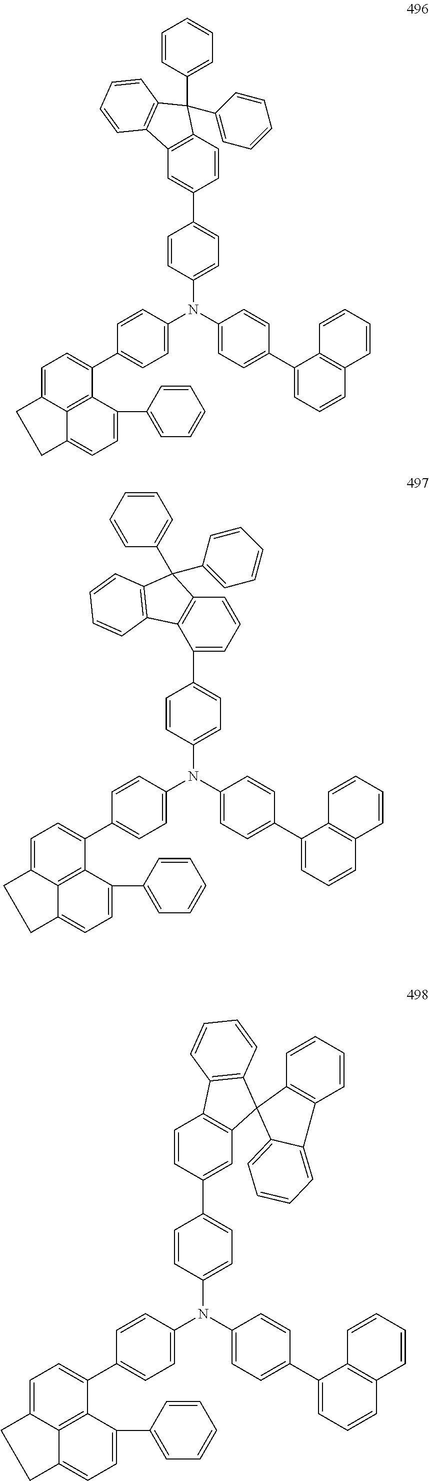 [SMILES: c1ccc(-c2ccc3c4c(ccc(-c5ccc(N(c6ccc(-c7ccc8c(c7)-c7ccccc7C8(c7ccccc7)c7ccccc7)cc6)c6ccc(-c7cccc8ccccc78)cc6)cc5)c24)CC3)cc1 ; c1ccc(-c2ccc3c4c(ccc(-c5ccc(N(c6ccc(-c7cccc8c7-c7ccccc7C8(c7ccccc7)c7ccccc7)cc6)c6ccc(-c7cccc8ccccc78)cc6)cc5)c24)CC3)cc1 ; c1ccc(-c2ccc3c4c(ccc(-c5ccc(N(c6ccc(-c7ccc8c(c7)C7(c9ccccc9-c9ccccc97)c7ccccc7-8)cc6)c6ccc(-c7cccc8ccccc78)cc6)cc5)c24)CC3)cc1]

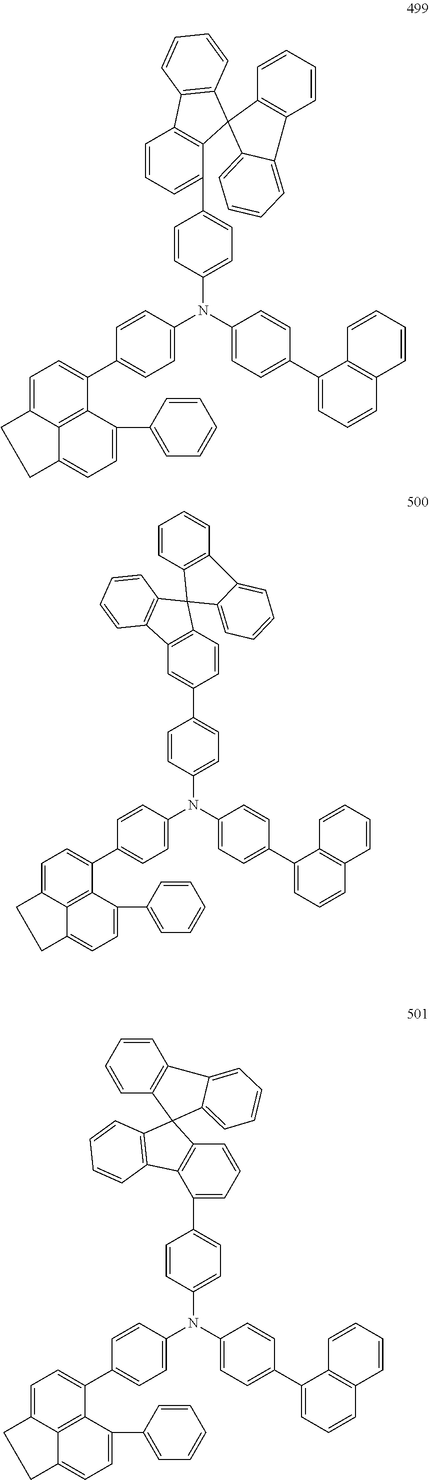 [SMILES: c1ccc(-c2ccc3c4c(ccc(-c5ccc(N(c6ccc(-c7cccc8c7C7(c9ccccc9-c9ccccc97)c7ccccc7-8)cc6)c6ccc(-c7cccc8ccccc78)cc6)cc5)c24)CC3)cc1 ; c1ccc(-c2ccc3c4c(ccc(-c5ccc(N(c6ccc(-c7ccc8c(c7)-c7ccccc7C87c8ccccc8-c8ccccc87)cc6)c6ccc(-c7cccc8ccccc78)cc6)cc5)c24)CC3)cc1 ; c1ccc(-c2ccc3c4c(ccc(-c5ccc(N(c6ccc(-c7cccc8c7-c7ccccc7C87c8ccccc8-c8ccccc87)cc6)c6ccc(-c7cccc8ccccc78)cc6)cc5)c24)CC3)cc1]

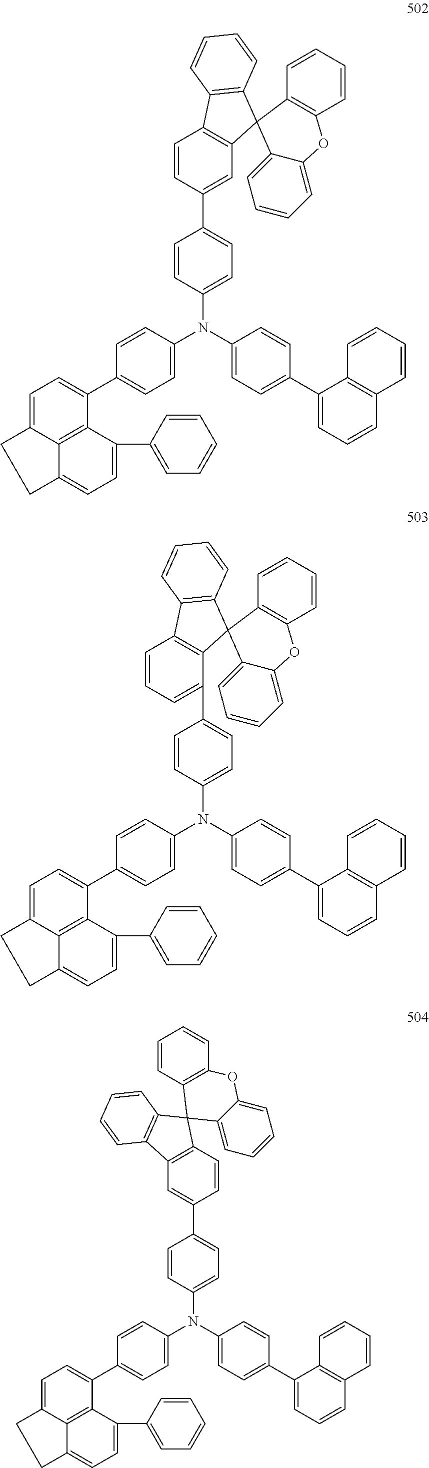 [SMILES: c1ccc(-c2ccc3c4c(ccc(-c5ccc(N(c6ccc(-c7ccc8c(c7)C7(c9ccccc9Oc9ccccc97)c7ccccc7-8)cc6)c6ccc(-c7cccc8ccccc78)cc6)cc5)c24)CC3)cc1 ; c1ccc(-c2ccc3c4c(ccc(-c5ccc(N(c6ccc(-c7cccc8c7C7(c9ccccc9Oc9ccccc97)c7ccccc7-8)cc6)c6ccc(-c7cccc8ccccc78)cc6)cc5)c24)CC3)cc1 ; c1ccc(-c2ccc3c4c(ccc(-c5ccc(N(c6ccc(-c7ccc8c(c7)-c7ccccc7C87c8ccccc8Oc8ccccc87)cc6)c6ccc(-c7cccc8ccccc78)cc6)cc5)c24)CC3)cc1]

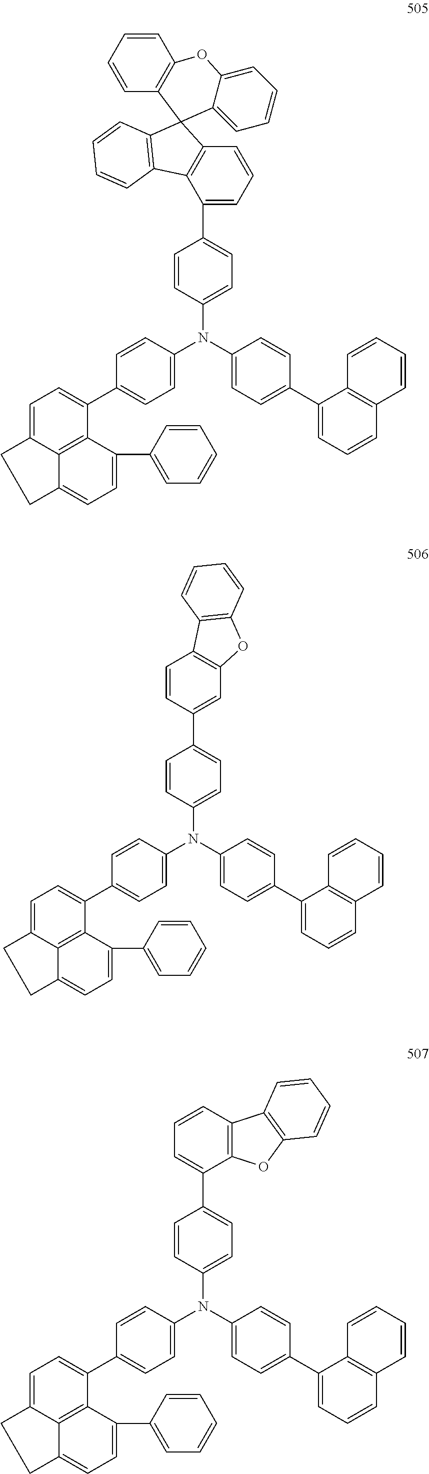 [SMILES: c1ccc(-c2ccc3c4c(ccc(-c5ccc(N(c6ccc(-c7cccc8c7-c7ccccc7C87c8ccccc8Oc8ccccc87)cc6)c6ccc(-c7cccc8ccccc78)cc6)cc5)c24)CC3)cc1 ; c1ccc(-c2ccc3c4c(ccc(-c5ccc(N(c6ccc(-c7ccc8c(c7)oc7ccccc78)cc6)c6ccc(-c7cccc8ccccc78)cc6)cc5)c24)CC3)cc1 ; c1ccc(-c2ccc3c4c(ccc(-c5ccc(N(c6ccc(-c7cccc8ccccc78)cc6)c6ccc(-c7cccc8c7oc7ccccc78)cc6)cc5)c24)CC3)cc1]

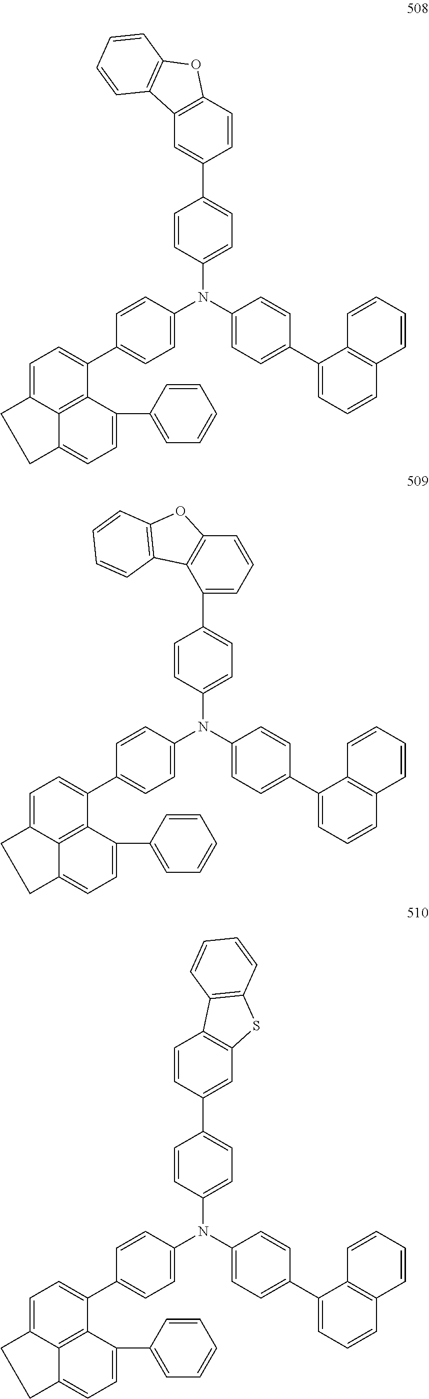 [SMILES: c1ccc(-c2ccc3c4c(ccc(-c5ccc(N(c6ccc(-c7ccc8oc9ccccc9c8c7)cc6)c6ccc(-c7cccc8ccccc78)cc6)cc5)c24)CC3)cc1 ; c1ccc(-c2ccc3c4c(ccc(-c5ccc(N(c6ccc(-c7cccc8ccccc78)cc6)c6ccc(-c7cccc8oc9ccccc9c78)cc6)cc5)c24)CC3)cc1 ; c1ccc(-c2ccc3c4c(ccc(-c5ccc(N(c6ccc(-c7ccc8c(c7)sc7ccccc78)cc6)c6ccc(-c7cccc8ccccc78)cc6)cc5)c24)CC3)cc1]

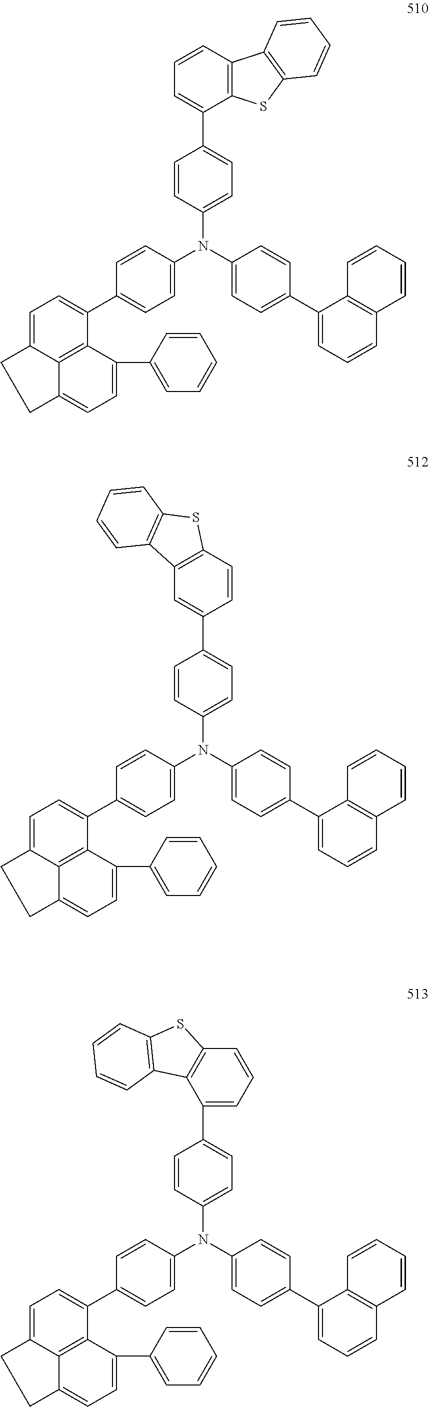 [SMILES: c1ccc(-c2ccc3c4c(ccc(-c5ccc(N(c6ccc(-c7cccc8ccccc78)cc6)c6ccc(-c7cccc8c7sc7ccccc78)cc6)cc5)c24)CC3)cc1 ; c1ccc(-c2ccc3c4c(ccc(-c5ccc(N(c6ccc(-c7ccc8sc9ccccc9c8c7)cc6)c6ccc(-c7cccc8ccccc78)cc6)cc5)c24)CC3)cc1 ; c1ccc(-c2ccc3c4c(ccc(-c5ccc(N(c6ccc(-c7cccc8ccccc78)cc6)c6ccc(-c7cccc8sc9ccccc9c78)cc6)cc5)c24)CC3)cc1]

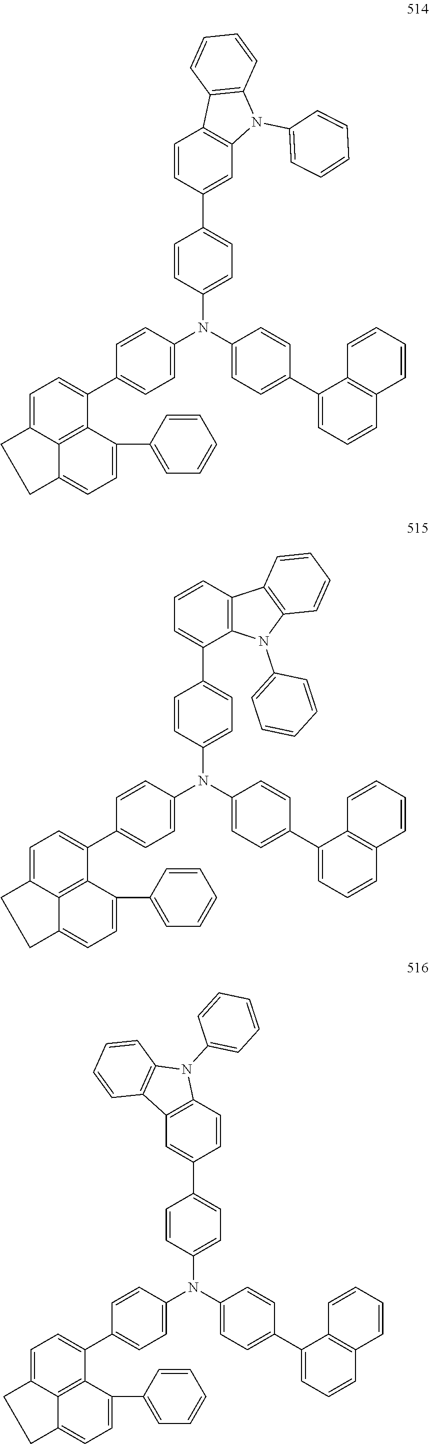 [SMILES: c1ccc(-c2ccc3c4c(ccc(-c5ccc(N(c6ccc(-c7ccc8c9ccccc9n(-c9ccccc9)c8c7)cc6)c6ccc(-c7cccc8ccccc78)cc6)cc5)c24)CC3)cc1 ; c1ccc(-c2ccc3c4c(ccc(-c5ccc(N(c6ccc(-c7cccc8ccccc78)cc6)c6ccc(-c7cccc8c9ccccc9n(-c9ccccc9)c78)cc6)cc5)c24)CC3)cc1 ; c1ccc(-c2ccc3c4c(ccc(-c5ccc(N(c6ccc(-c7ccc8c(c7)c7ccccc7n8-c7ccccc7)cc6)c6ccc(-c7cccc8ccccc78)cc6)cc5)c24)CC3)cc1]

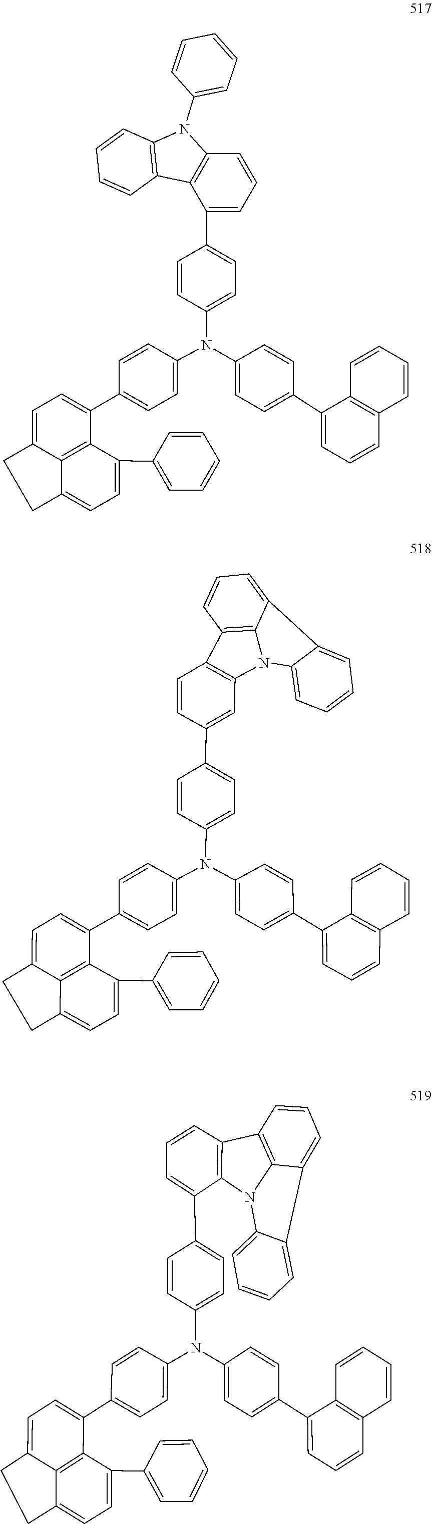 [SMILES: c1ccc(-c2ccc3c4c(ccc(-c5ccc(N(c6ccc(-c7cccc8ccccc78)cc6)c6ccc(-c7cccc8c7c7ccccc7n8-c7ccccc7)cc6)cc5)c24)CC3)cc1 ; c1ccc(-c2ccc3c4c(ccc(-c5ccc(N(c6ccc(-c7ccc8c9cccc%10c%11ccccc%11n(c8c7)c%109)cc6)c6ccc(-c7cccc8ccccc78)cc6)cc5)c24)CC3)cc1 ; c1ccc(-c2ccc3c4c(ccc(-c5ccc(N(c6ccc(-c7cccc8ccccc78)cc6)c6ccc(-c7cccc8c9cccc%10c%11ccccc%11n(c78)c%109)cc6)cc5)c24)CC3)cc1]

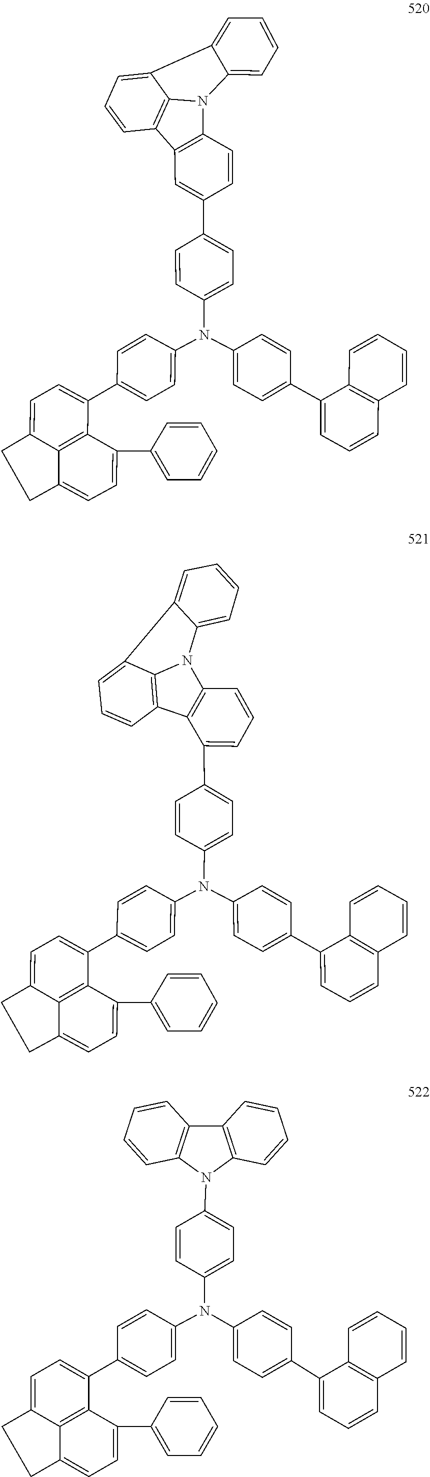 [SMILES: c1ccc(-c2ccc3c4c(ccc(-c5ccc(N(c6ccc(-c7ccc8c(c7)c7cccc9c%10ccccc%10n8c97)cc6)c6ccc(-c7cccc8ccccc78)cc6)cc5)c24)CC3)cc1 ; c1ccc(-c2ccc3c4c(ccc(-c5ccc(N(c6ccc(-c7cccc8ccccc78)cc6)c6ccc(-c7cccc8c7c7cccc9c%10ccccc%10n8c97)cc6)cc5)c24)CC3)cc1 ; c1ccc(-c2ccc3c4c(ccc(-c5ccc(N(c6ccc(-c7cccc8ccccc78)cc6)c6ccc(-n7c8ccccc8c8ccccc87)cc6)cc5)c24)CC3)cc1]

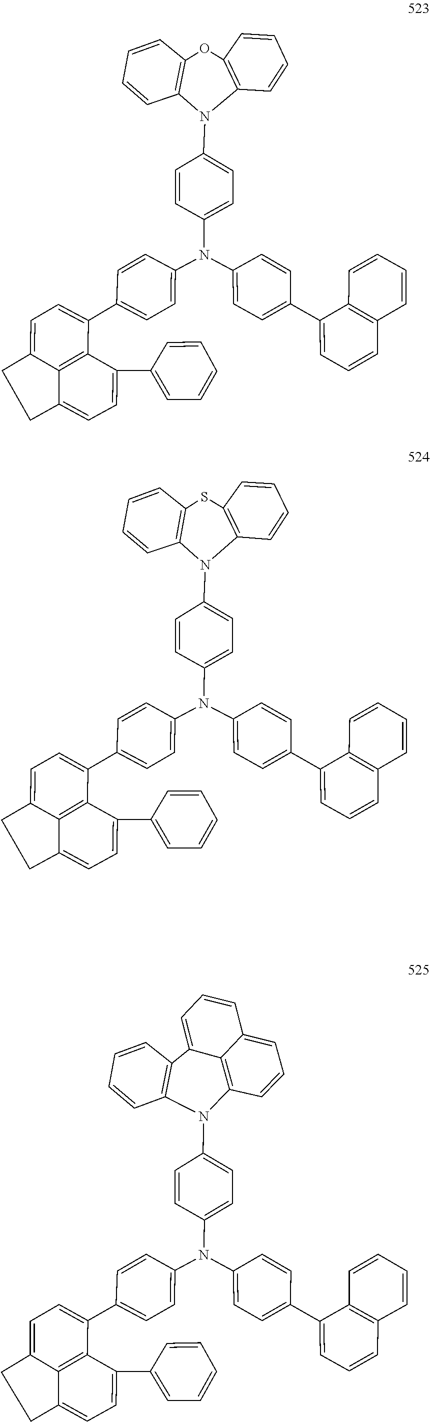 [SMILES: c1ccc(-c2ccc3c4c(ccc(-c5ccc(N(c6ccc(-c7cccc8ccccc78)cc6)c6ccc(N7c8ccccc8Oc8ccccc87)cc6)cc5)c24)CC3)cc1 ; c1ccc(-c2ccc3c4c(ccc(-c5ccc(N(c6ccc(-c7cccc8ccccc78)cc6)c6ccc(N7c8ccccc8Sc8ccccc87)cc6)cc5)c24)CC3)cc1 ; c1ccc(-c2ccc3c4c(ccc(-c5ccc(N(c6ccc(-c7cccc8ccccc78)cc6)c6ccc(N7c8ccccc8-c8cccc9cccc7c89)cc6)cc5)c24)CC3)cc1]

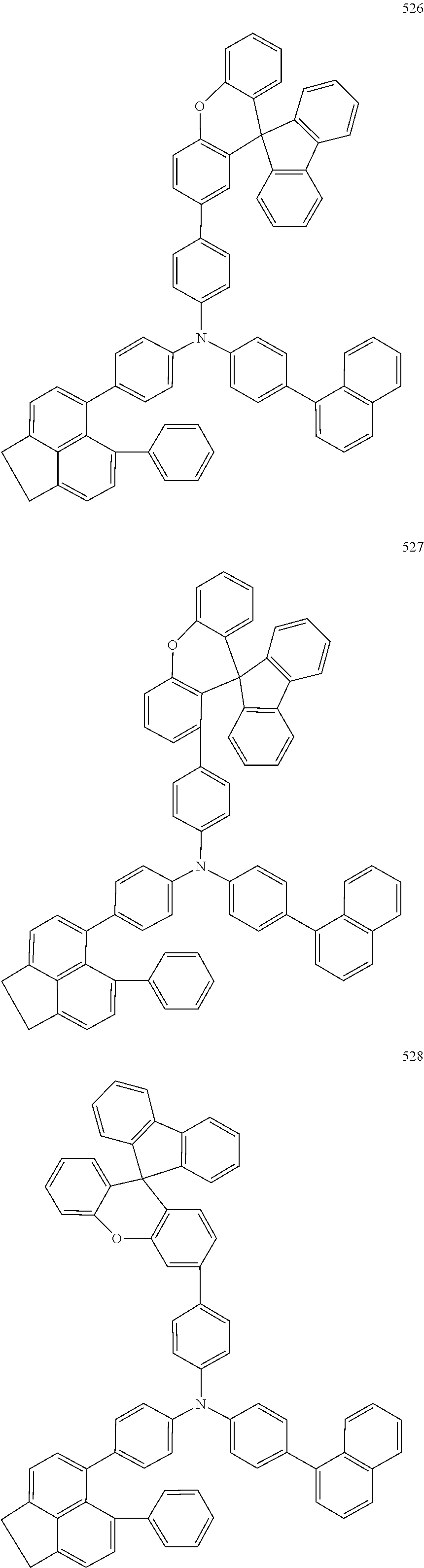 [SMILES: c1ccc(-c2ccc3c4c(ccc(-c5ccc(N(c6ccc(-c7ccc8c(c7)C7(c9ccccc9O8)c8ccccc8-c8ccccc87)cc6)c6ccc(-c7cccc8ccccc78)cc6)cc5)c24)CC3)cc1 ; c1ccc(-c2ccc3c4c(ccc(-c5ccc(N(c6ccc(-c7cccc8c7C7(c9ccccc9O8)c8ccccc8-c8ccccc87)cc6)c6ccc(-c7cccc8ccccc78)cc6)cc5)c24)CC3)cc1 ; c1ccc(-c2ccc3c4c(ccc(-c5ccc(N(c6ccc(-c7ccc8c(c7)Oc7ccccc7C87c8ccccc8-c8ccccc87)cc6)c6ccc(-c7cccc8ccccc78)cc6)cc5)c24)CC3)cc1]

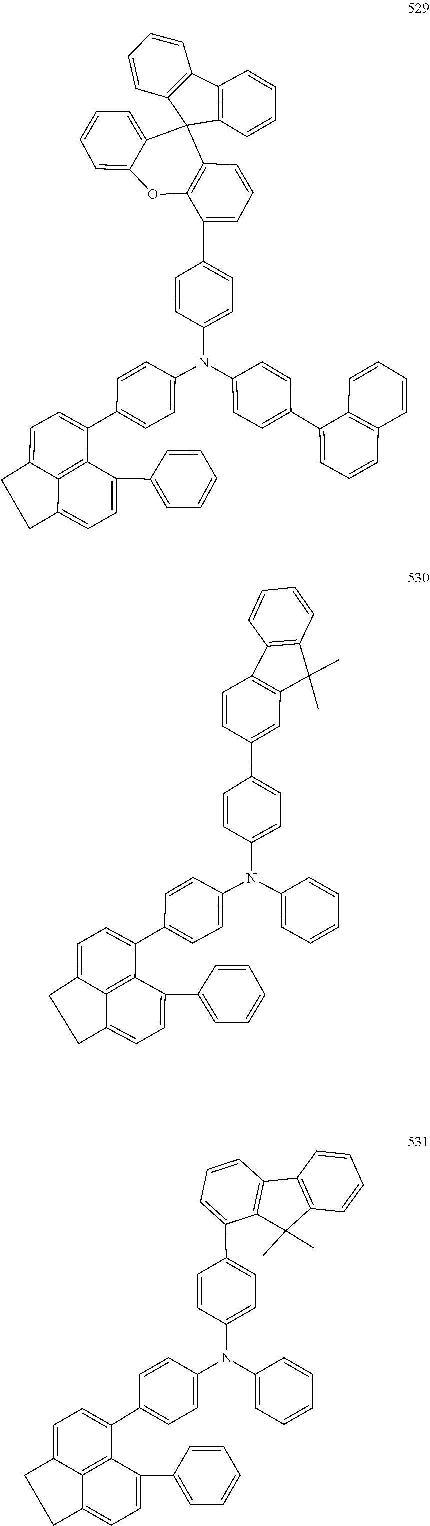 [SMILES: c1ccc(-c2ccc3c4c(ccc(-c5ccc(N(c6ccc(-c7cccc8c7Oc7ccccc7C87c8ccccc8-c8ccccc87)cc6)c6ccc(-c7cccc8ccccc78)cc6)cc5)c24)CC3)cc1 ; CC1(C)c2ccccc2-c2ccc(-c3ccc(N(c4ccccc4)c4ccc(-c5ccc6c7c(ccc(-c8ccccc8)c57)CC6)cc4)cc3)cc21 ; CC1(C)c2ccccc2-c2cccc(-c3ccc(N(c4ccccc4)c4ccc(-c5ccc6c7c(ccc(-c8ccccc8)c57)CC6)cc4)cc3)c21]

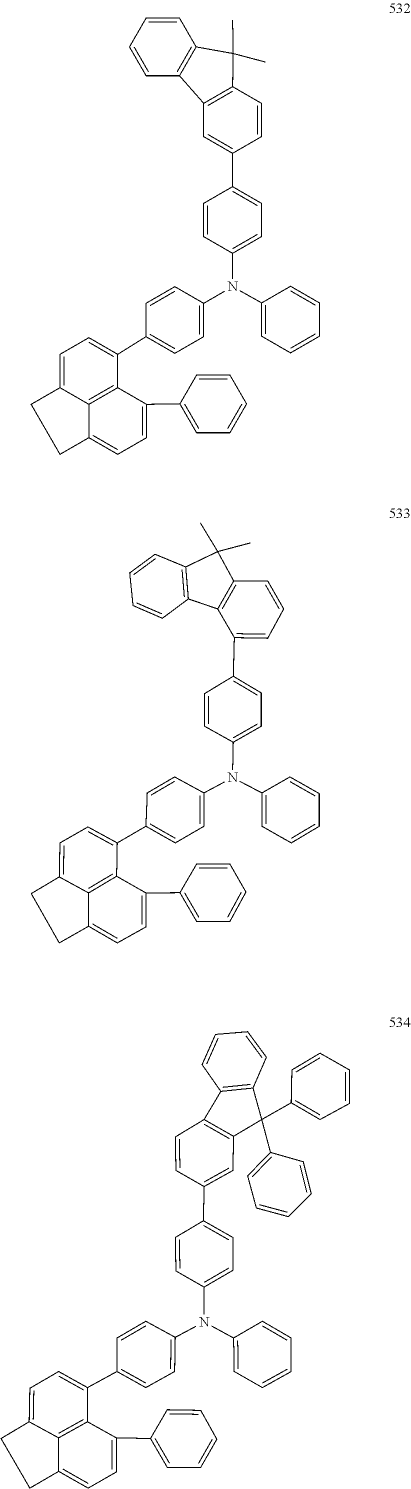 [SMILES: CC1(C)c2ccccc2-c2cc(-c3ccc(N(c4ccccc4)c4ccc(-c5ccc6c7c(ccc(-c8ccccc8)c57)CC6)cc4)cc3)ccc21 ; CC1(C)c2ccccc2-c2c(-c3ccc(N(c4ccccc4)c4ccc(-c5ccc6c7c(ccc(-c8ccccc8)c57)CC6)cc4)cc3)cccc21 ; c1ccc(-c2ccc3c4c(ccc(-c5ccc(N(c6ccccc6)c6ccc(-c7ccc8c(c7)C(c7ccccc7)(c7ccccc7)c7ccccc7-8)cc6)cc5)c24)CC3)cc1]

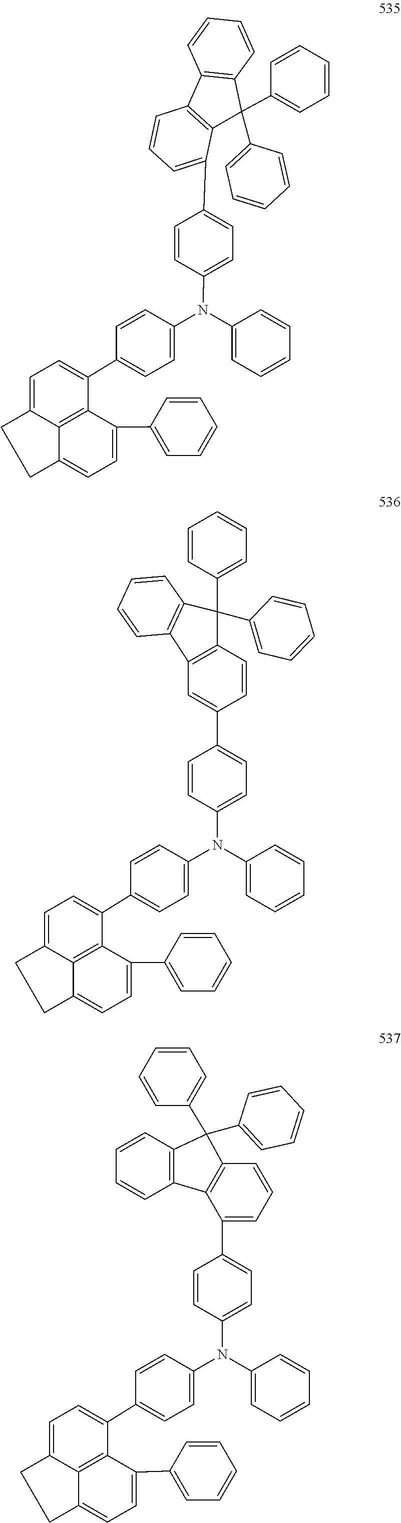 [SMILES: c1ccc(-c2ccc3c4c(ccc(-c5ccc(N(c6ccccc6)c6ccc(-c7cccc8c7C(c7ccccc7)(c7ccccc7)c7ccccc7-8)cc6)cc5)c24)CC3)cc1 ; c1ccc(-c2ccc3c4c(ccc(-c5ccc(N(c6ccccc6)c6ccc(-c7ccc8c(c7)-c7ccccc7C8(c7ccccc7)c7ccccc7)cc6)cc5)c24)CC3)cc1 ; c1ccc(-c2ccc3c4c(ccc(-c5ccc(N(c6ccccc6)c6ccc(-c7cccc8c7-c7ccccc7C8(c7ccccc7)c7ccccc7)cc6)cc5)c24)CC3)cc1]

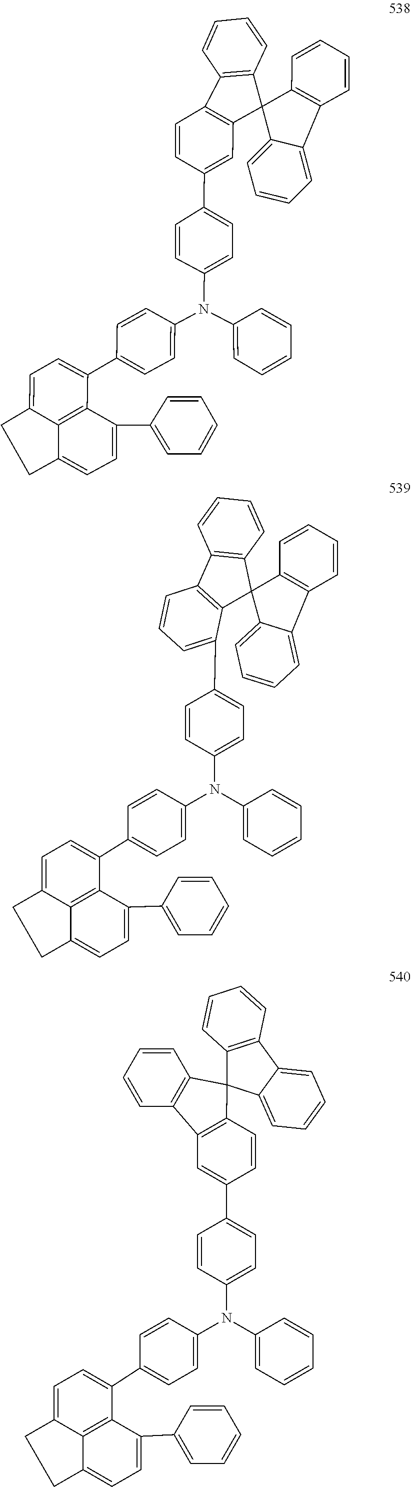 [SMILES: c1ccc(-c2ccc3c4c(ccc(-c5ccc(N(c6ccccc6)c6ccc(-c7ccc8c(c7)C7(c9ccccc9-c9ccccc97)c7ccccc7-8)cc6)cc5)c24)CC3)cc1 ; c1ccc(-c2ccc3c4c(ccc(-c5ccc(N(c6ccccc6)c6ccc(-c7cccc8c7C7(c9ccccc9-c9ccccc97)c7ccccc7-8)cc6)cc5)c24)CC3)cc1 ; c1ccc(-c2ccc3c4c(ccc(-c5ccc(N(c6ccccc6)c6ccc(-c7ccc8c(c7)-c7ccccc7C87c8ccccc8-c8ccccc87)cc6)cc5)c24)CC3)cc1]

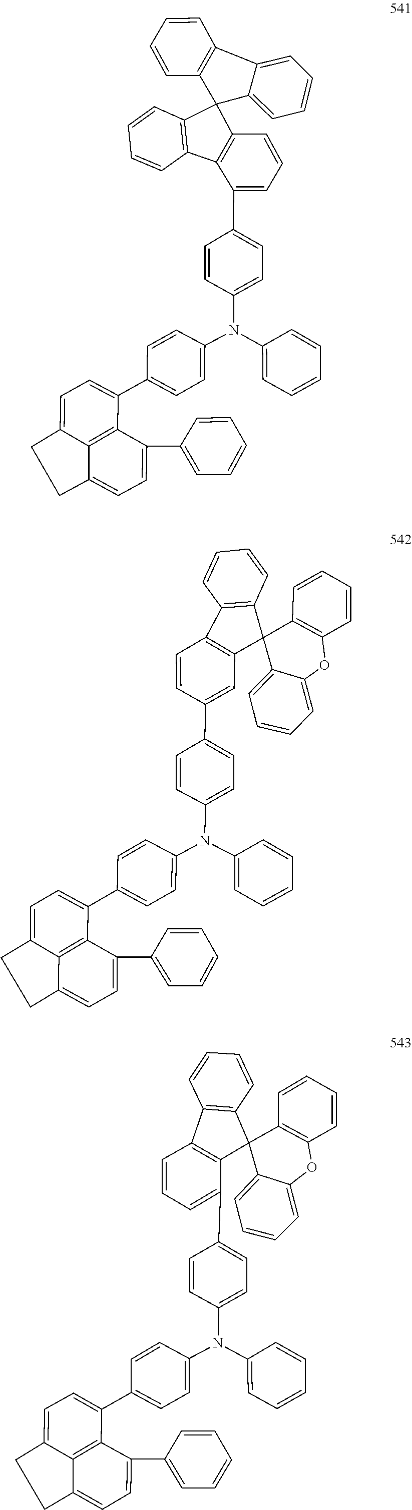 [SMILES: c1ccc(-c2ccc3c4c(ccc(-c5ccc(N(c6ccccc6)c6ccc(-c7cccc8c7-c7ccccc7C87c8ccccc8-c8ccccc87)cc6)cc5)c24)CC3)cc1 ; c1ccc(-c2ccc3c4c(ccc(-c5ccc(N(c6ccccc6)c6ccc(-c7ccc8c(c7)C7(c9ccccc9Oc9ccccc97)c7ccccc7-8)cc6)cc5)c24)CC3)cc1 ; c1ccc(-c2ccc3c4c(ccc(-c5ccc(N(c6ccccc6)c6ccc(-c7cccc8c7C7(c9ccccc9Oc9ccccc97)c7ccccc7-8)cc6)cc5)c24)CC3)cc1]

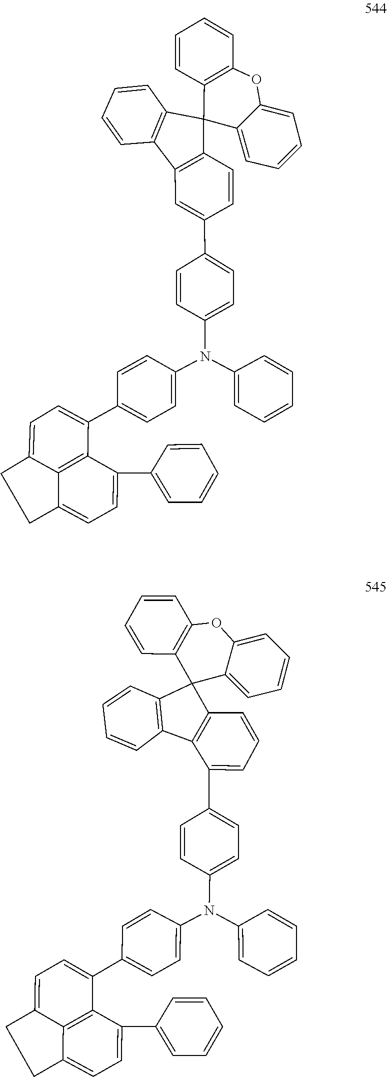 [SMILES: c1ccc(-c2ccc3c4c(ccc(-c5ccc(N(c6ccccc6)c6ccc(-c7ccc8c(c7)-c7ccccc7C87c8ccccc8Oc8ccccc87)cc6)cc5)c24)CC3)cc1 ; c1ccc(-c2ccc3c4c(ccc(-c5ccc(N(c6ccccc6)c6ccc(-c7cccc8c7-c7ccccc7C87c8ccccc8Oc8ccccc87)cc6)cc5)c24)CC3)cc1]

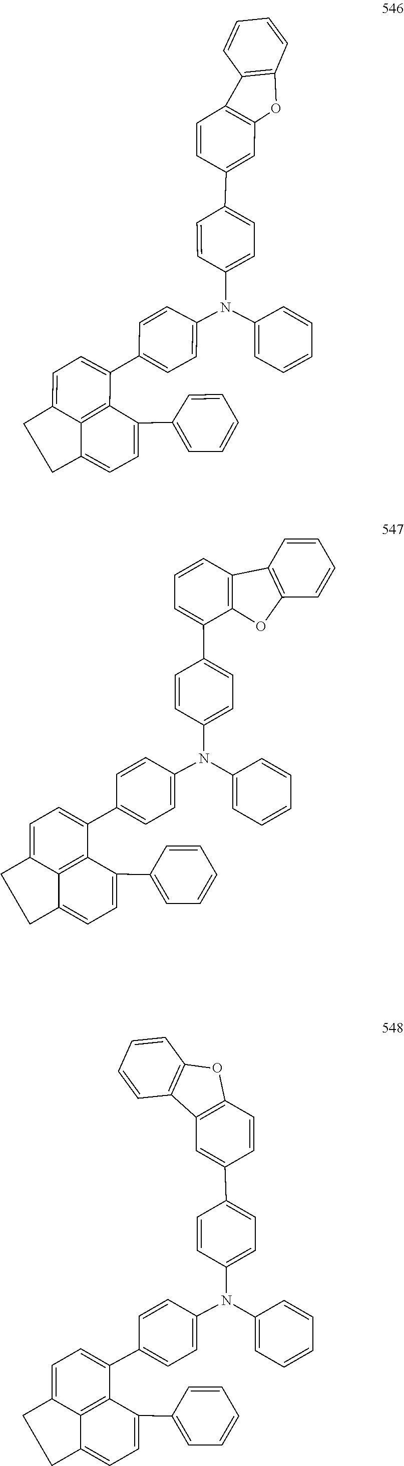 [SMILES: c1ccc(-c2ccc3c4c(ccc(-c5ccc(N(c6ccccc6)c6ccc(-c7ccc8c(c7)oc7ccccc78)cc6)cc5)c24)CC3)cc1 ; c1ccc(-c2ccc3c4c(ccc(-c5ccc(N(c6ccccc6)c6ccc(-c7cccc8c7oc7ccccc78)cc6)cc5)c24)CC3)cc1 ; c1ccc(-c2ccc3c4c(ccc(-c5ccc(N(c6ccccc6)c6ccc(-c7ccc8oc9ccccc9c8c7)cc6)cc5)c24)CC3)cc1]

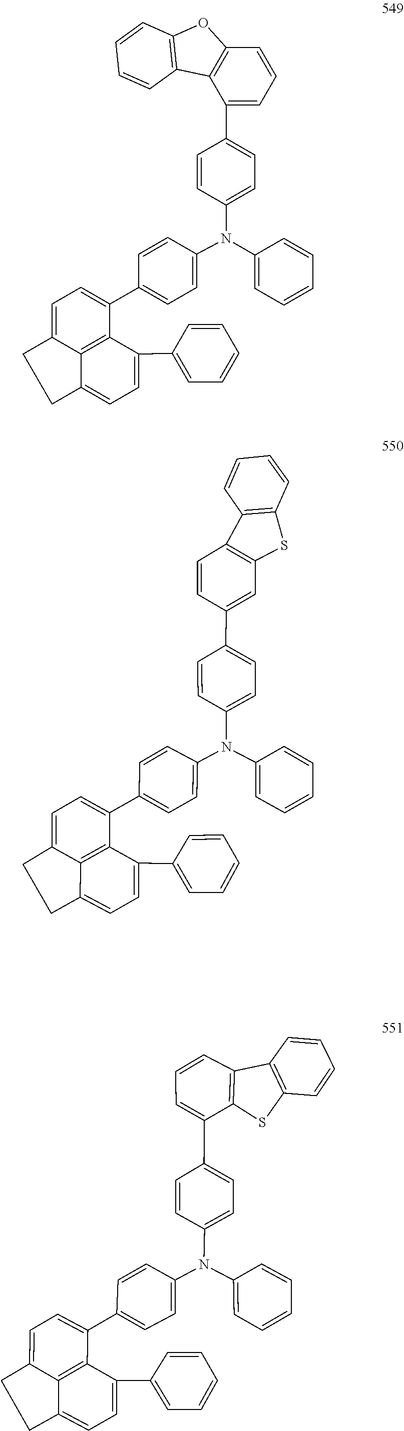 [SMILES: c1ccc(-c2ccc3c4c(ccc(-c5ccc(N(c6ccccc6)c6ccc(-c7cccc8oc9ccccc9c78)cc6)cc5)c24)CC3)cc1 ; c1ccc(-c2ccc3c4c(ccc(-c5ccc(N(c6ccccc6)c6ccc(-c7ccc8c(c7)sc7ccccc78)cc6)cc5)c24)CC3)cc1 ; c1ccc(-c2ccc3c4c(ccc(-c5ccc(N(c6ccccc6)c6ccc(-c7cccc8c7sc7ccccc78)cc6)cc5)c24)CC3)cc1]

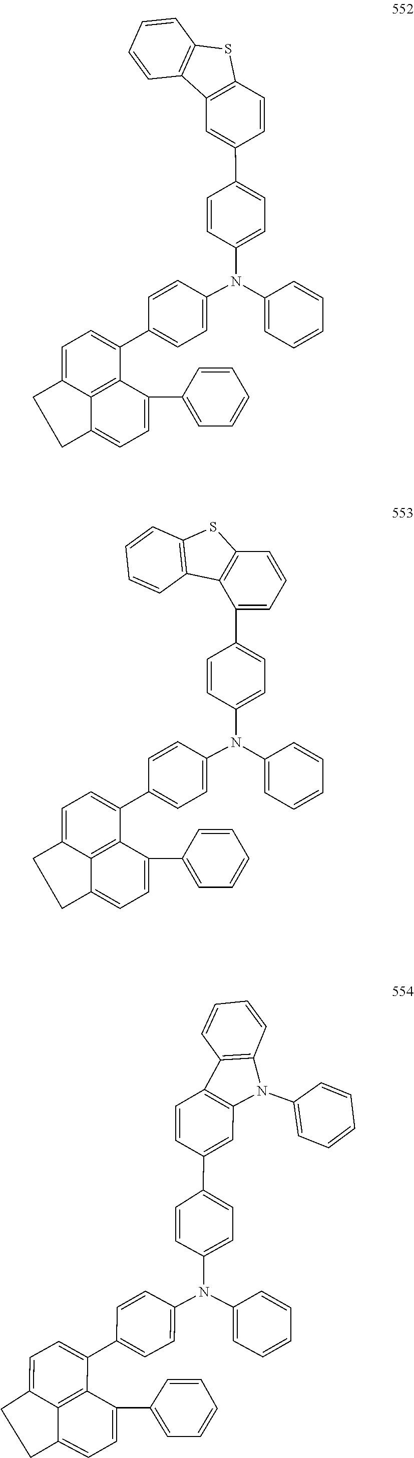 [SMILES: c1ccc(-c2ccc3c4c(ccc(-c5ccc(N(c6ccccc6)c6ccc(-c7ccc8sc9ccccc9c8c7)cc6)cc5)c24)CC3)cc1 ; c1ccc(-c2ccc3c4c(ccc(-c5ccc(N(c6ccccc6)c6ccc(-c7cccc8sc9ccccc9c78)cc6)cc5)c24)CC3)cc1 ; c1ccc(-c2ccc3c4c(ccc(-c5ccc(N(c6ccccc6)c6ccc(-c7ccc8c9ccccc9n(-c9ccccc9)c8c7)cc6)cc5)c24)CC3)cc1]

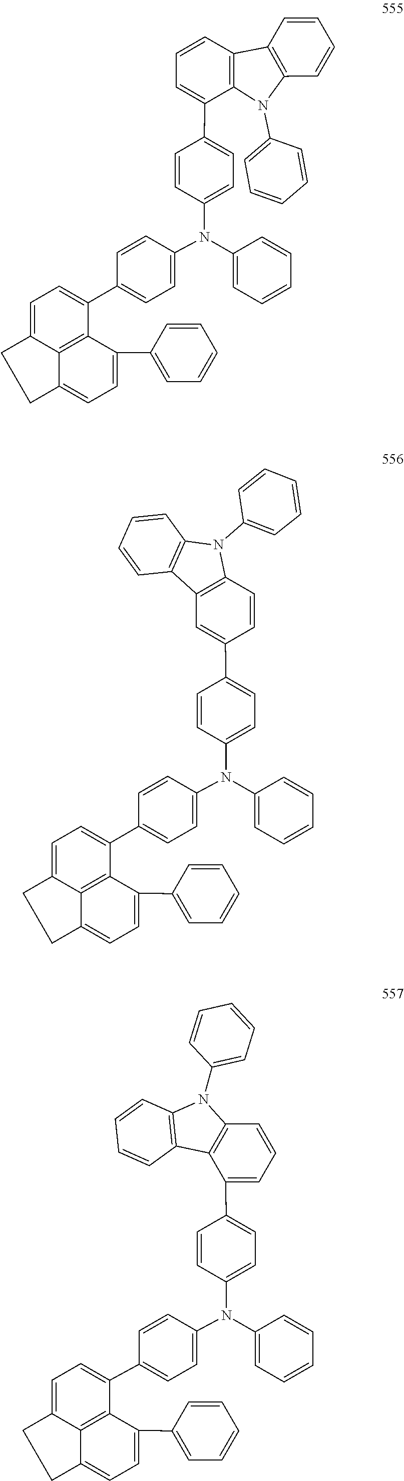 [SMILES: c1ccc(-c2ccc3c4c(ccc(-c5ccc(N(c6ccccc6)c6ccc(-c7cccc8c9ccccc9n(-c9ccccc9)c78)cc6)cc5)c24)CC3)cc1 ; c1ccc(-c2ccc3c4c(ccc(-c5ccc(N(c6ccccc6)c6ccc(-c7ccc8c(c7)c7ccccc7n8-c7ccccc7)cc6)cc5)c24)CC3)cc1 ; c1ccc(-c2ccc3c4c(ccc(-c5ccc(N(c6ccccc6)c6ccc(-c7cccc8c7c7ccccc7n8-c7ccccc7)cc6)cc5)c24)CC3)cc1]

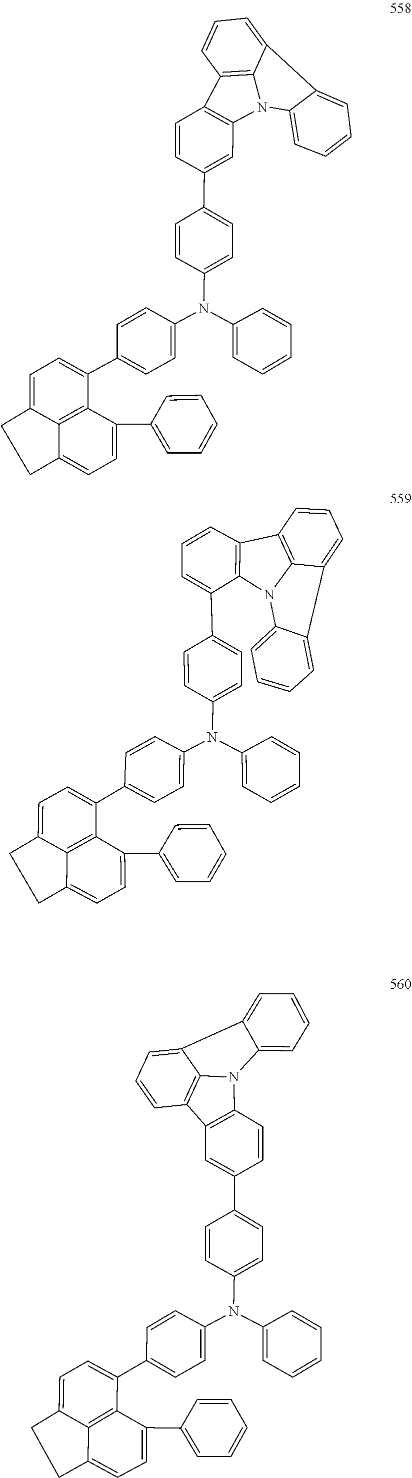 [SMILES: c1ccc(-c2ccc3c4c(ccc(-c5ccc(N(c6ccccc6)c6ccc(-c7ccc8c9cccc%10c%11ccccc%11n(c8c7)c%109)cc6)cc5)c24)CC3)cc1 ; c1ccc(-c2ccc3c4c(ccc(-c5ccc(N(c6ccccc6)c6ccc(-c7cccc8c9cccc%10c%11ccccc%11n(c78)c%109)cc6)cc5)c24)CC3)cc1 ; c1ccc(-c2ccc3c4c(ccc(-c5ccc(N(c6ccccc6)c6ccc(-c7ccc8c(c7)c7cccc9c%10ccccc%10n8c97)cc6)cc5)c24)CC3)cc1]

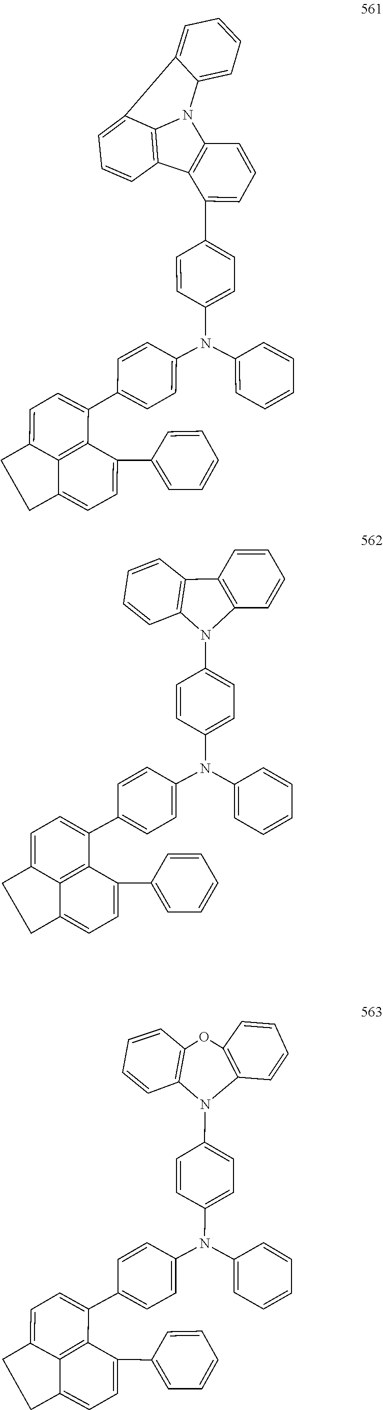 [SMILES: c1ccc(-c2ccc3c4c(ccc(-c5ccc(N(c6ccccc6)c6ccc(-c7cccc8c7c7cccc9c%10ccccc%10n8c97)cc6)cc5)c24)CC3)cc1 ; c1ccc(-c2ccc3c4c(ccc(-c5ccc(N(c6ccccc6)c6ccc(-n7c8ccccc8c8ccccc87)cc6)cc5)c24)CC3)cc1 ; c1ccc(-c2ccc3c4c(ccc(-c5ccc(N(c6ccccc6)c6ccc(N7c8ccccc8Oc8ccccc87)cc6)cc5)c24)CC3)cc1]

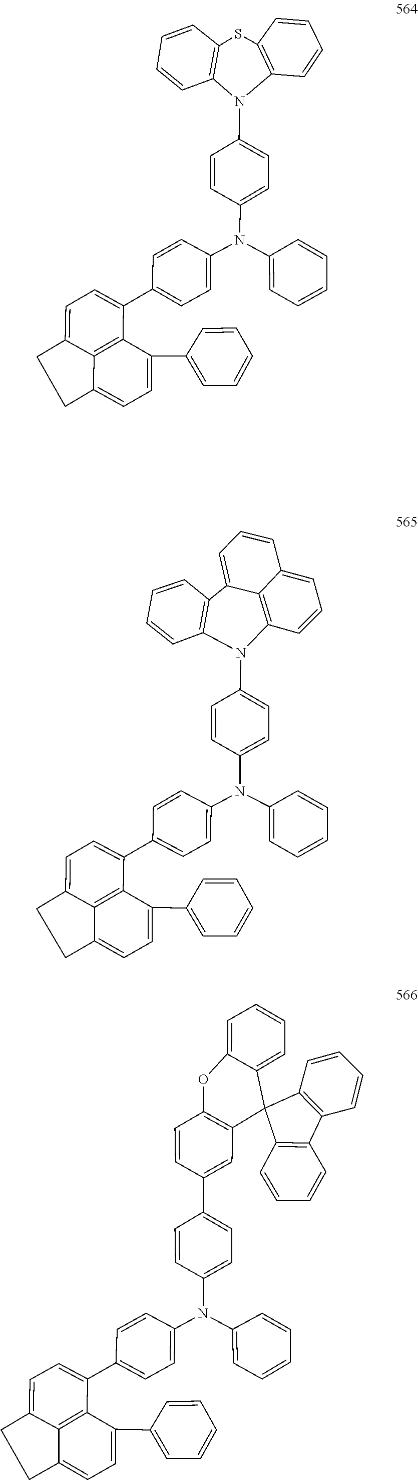 [SMILES: c1ccc(-c2ccc3c4c(ccc(-c5ccc(N(c6ccccc6)c6ccc(N7c8ccccc8Sc8ccccc87)cc6)cc5)c24)CC3)cc1 ; c1ccc(-c2ccc3c4c(ccc(-c5ccc(N(c6ccccc6)c6ccc(N7c8ccccc8-c8cccc9cccc7c89)cc6)cc5)c24)CC3)cc1 ; c1ccc(-c2ccc3c4c(ccc(-c5ccc(N(c6ccccc6)c6ccc(-c7ccc8c(c7)C7(c9ccccc9O8)c8ccccc8-c8ccccc87)cc6)cc5)c24)CC3)cc1]

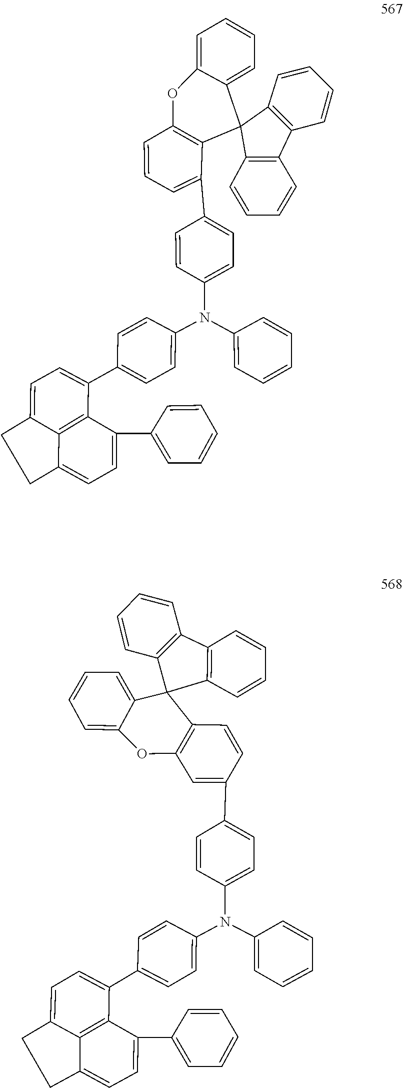 [SMILES: c1ccc(-c2ccc3c4c(ccc(-c5ccc(N(c6ccccc6)c6ccc(-c7cccc8c7C7(c9ccccc9O8)c8ccccc8-c8ccccc87)cc6)cc5)c24)CC3)cc1 ; c1ccc(-c2ccc3c4c(ccc(-c5ccc(N(c6ccccc6)c6ccc(-c7ccc8c(c7)Oc7ccccc7C87c8ccccc8-c8ccccc87)cc6)cc5)c24)CC3)cc1]

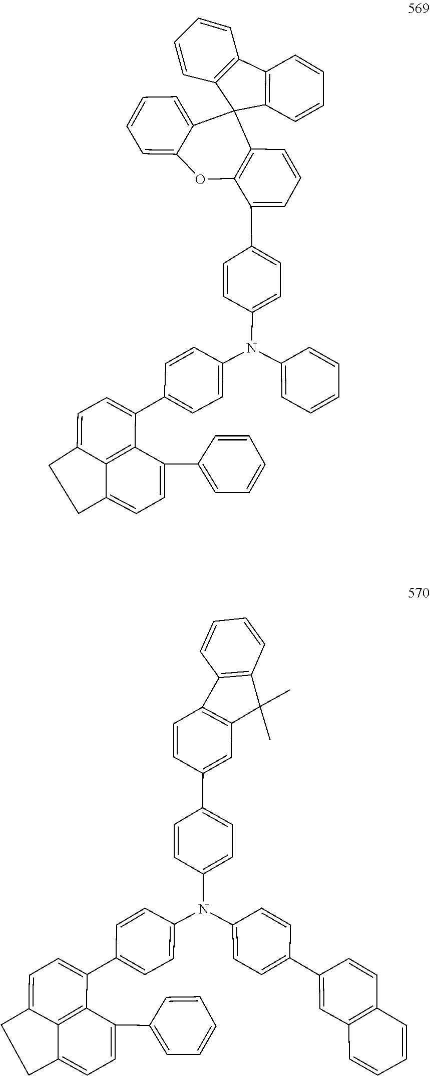 [SMILES: c1ccc(-c2ccc3c4c(ccc(-c5ccc(N(c6ccccc6)c6ccc(-c7cccc8c7Oc7ccccc7C87c8ccccc8-c8ccccc87)cc6)cc5)c24)CC3)cc1 ; CC1(C)c2ccccc2-c2ccc(-c3ccc(N(c4ccc(-c5ccc6ccccc6c5)cc4)c4ccc(-c5ccc6c7c(ccc(-c8ccccc8)c57)CC6)cc4)cc3)cc21]

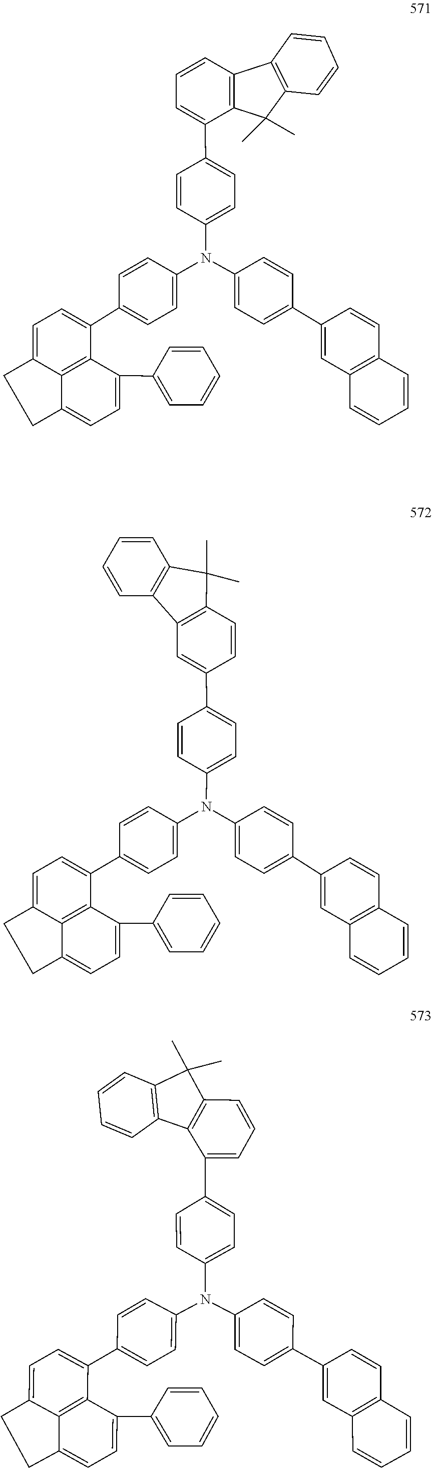 [SMILES: CC1(C)c2ccccc2-c2cccc(-c3ccc(N(c4ccc(-c5ccc6ccccc6c5)cc4)c4ccc(-c5ccc6c7c(ccc(-c8ccccc8)c57)CC6)cc4)cc3)c21 ; CC1(C)c2ccccc2-c2cc(-c3ccc(N(c4ccc(-c5ccc6ccccc6c5)cc4)c4ccc(-c5ccc6c7c(ccc(-c8ccccc8)c57)CC6)cc4)cc3)ccc21 ; CC1(C)c2ccccc2-c2c(-c3ccc(N(c4ccc(-c5ccc6ccccc6c5)cc4)c4ccc(-c5ccc6c7c(ccc(-c8ccccc8)c57)CC6)cc4)cc3)cccc21]

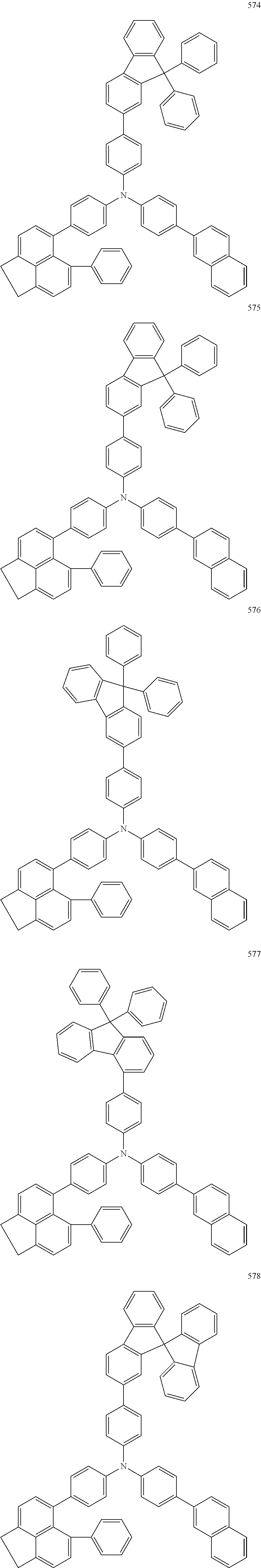

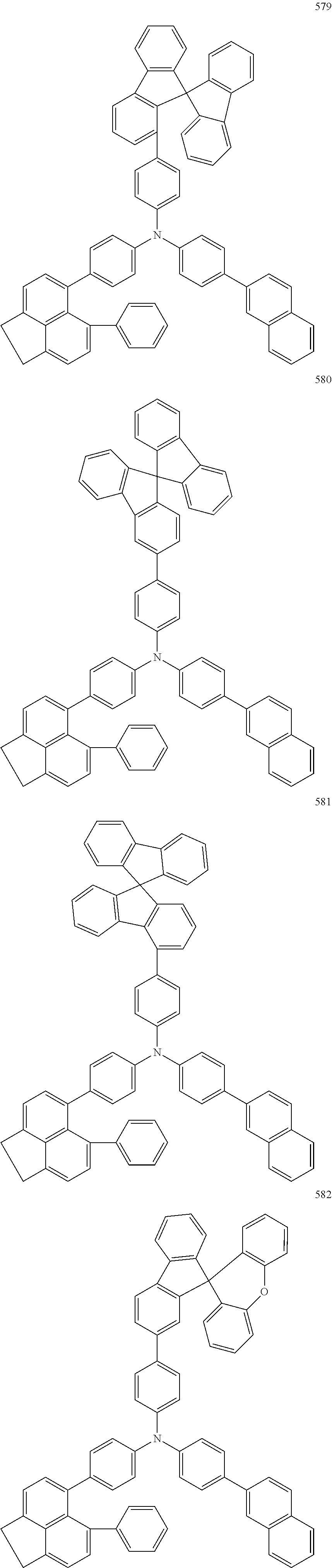

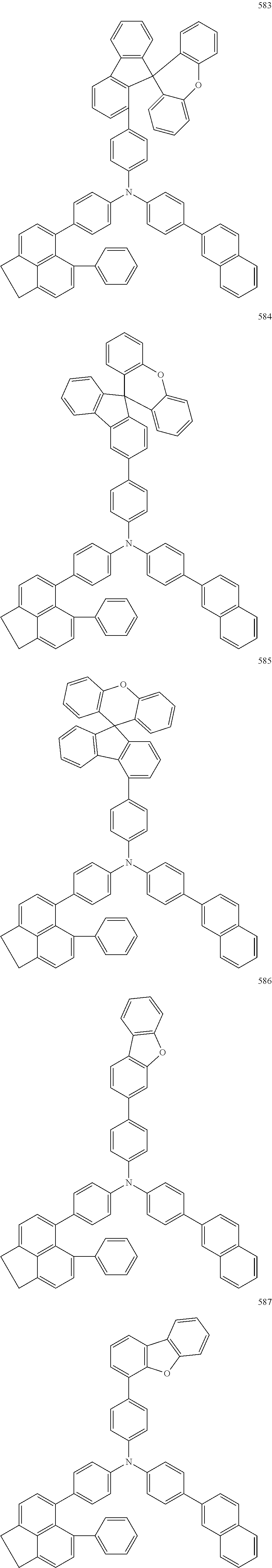

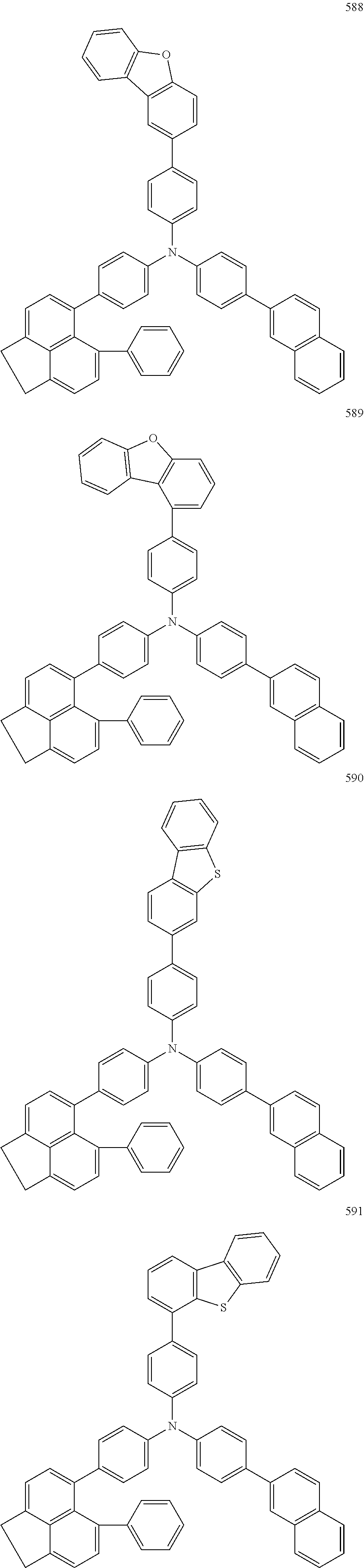 [SMILES: c1ccc(-c2ccc3c4c(ccc(-c5ccc(N(c6ccc(-c7ccc8ccccc8c7)cc6)c6ccc(-c7ccc8oc9ccccc9c8c7)cc6)cc5)c24)CC3)cc1 ; c1ccc(-c2ccc3c4c(ccc(-c5ccc(N(c6ccc(-c7ccc8ccccc8c7)cc6)c6ccc(-c7cccc8oc9ccccc9c78)cc6)cc5)c24)CC3)cc1 ; c1ccc(-c2ccc3c4c(ccc(-c5ccc(N(c6ccc(-c7ccc8ccccc8c7)cc6)c6ccc(-c7ccc8c(c7)sc7ccccc78)cc6)cc5)c24)CC3)cc1 ; c1ccc(-c2ccc3c4c(ccc(-c5ccc(N(c6ccc(-c7ccc8ccccc8c7)cc6)c6ccc(-c7cccc8c7sc7ccccc78)cc6)cc5)c24)CC3)cc1]

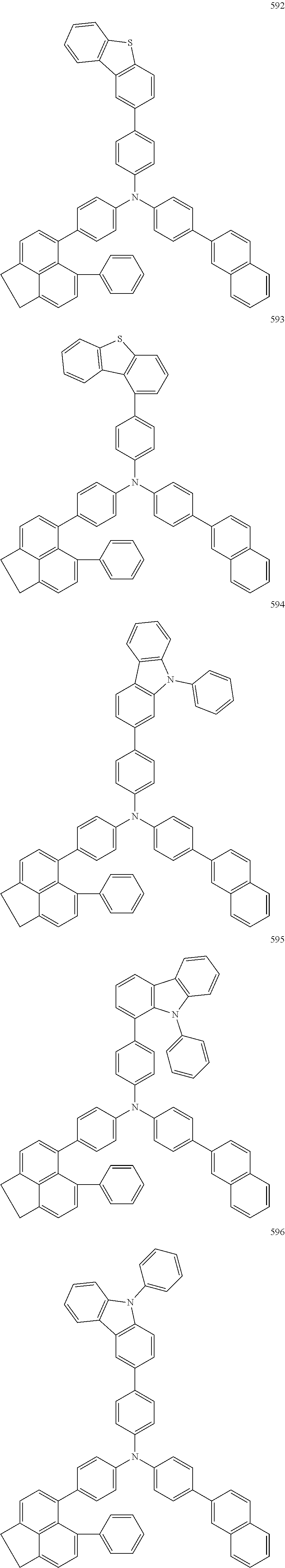

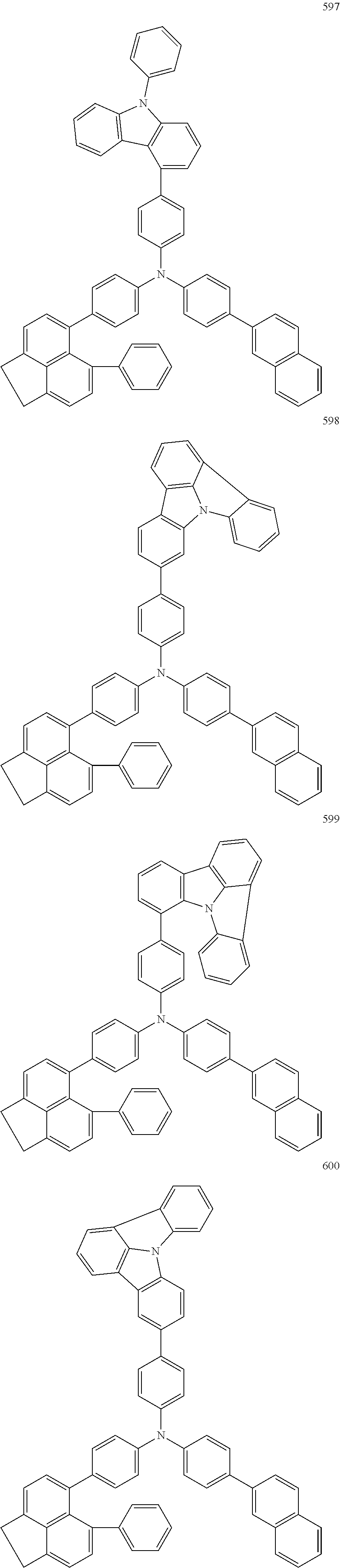 [SMILES: c1ccc(-c2ccc3c4c(ccc(-c5ccc(N(c6ccc(-c7ccc8ccccc8c7)cc6)c6ccc(-c7cccc8c7c7ccccc7n8-c7ccccc7)cc6)cc5)c24)CC3)cc1 ; c1ccc(-c2ccc3c4c(ccc(-c5ccc(N(c6ccc(-c7ccc8ccccc8c7)cc6)c6ccc(-c7ccc8c9cccc%10c%11ccccc%11n(c8c7)c%109)cc6)cc5)c24)CC3)cc1 ; c1ccc(-c2ccc3c4c(ccc(-c5ccc(N(c6ccc(-c7ccc8ccccc8c7)cc6)c6ccc(-c7cccc8c9cccc%10c%11ccccc%11n(c78)c%109)cc6)cc5)c24)CC3)cc1 ; c1ccc(-c2ccc3c4c(ccc(-c5ccc(N(c6ccc(-c7ccc8ccccc8c7)cc6)c6ccc(-c7ccc8c(c7)c7cccc9c%10ccccc%10n8c97)cc6)cc5)c24)CC3)cc1]

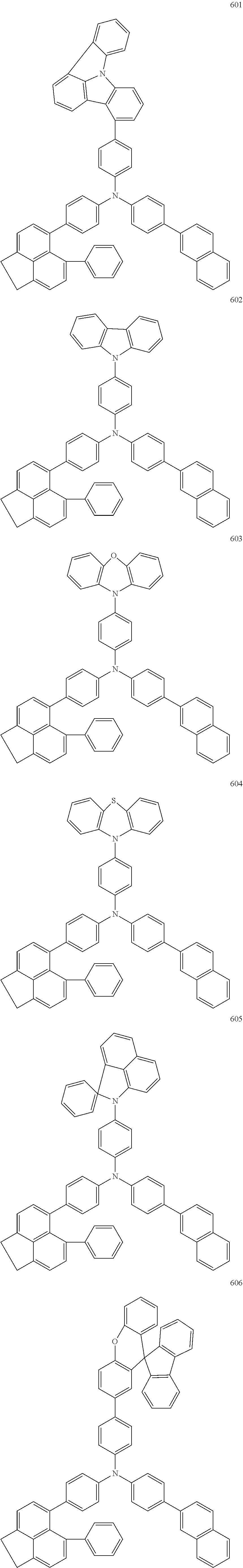

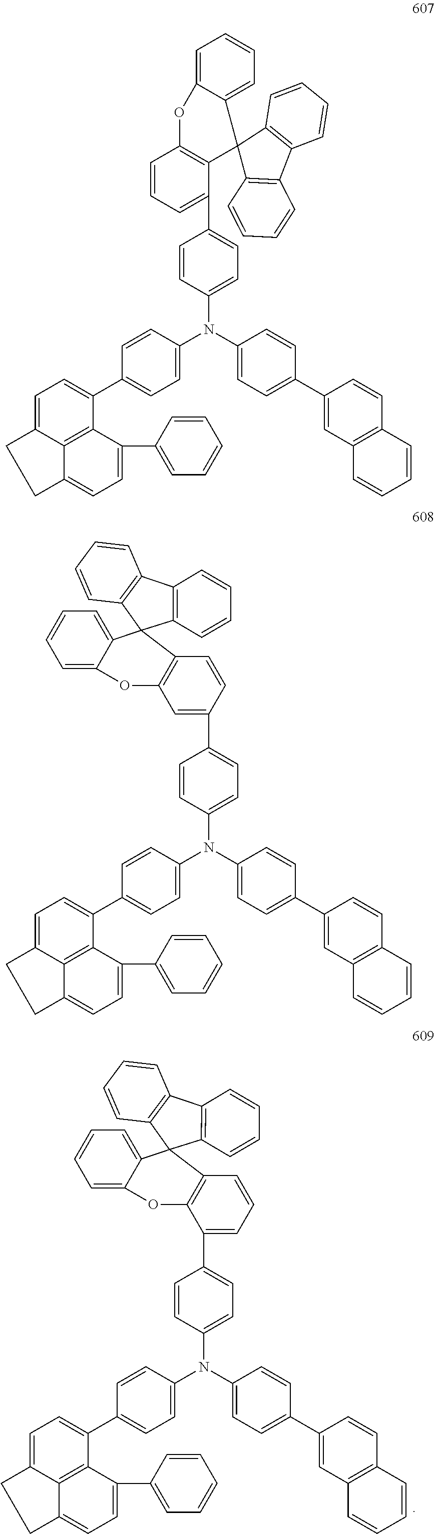

Furthermore, when Ar₃ is selected from

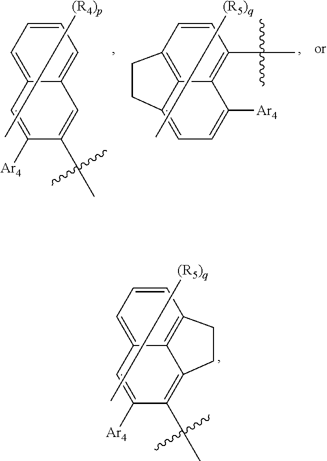

hydrogens on the benzene ring directly bonded to Ar₃ in the Ar₄ group may be partially or fully substituted with deuterium. Additionally, hydrogens on the benzene ring connecting the bonding site to the nitrogen atom in the main structure may also be substituted with deuterium to enhance device lifetime. For example, the structure may be represented as:

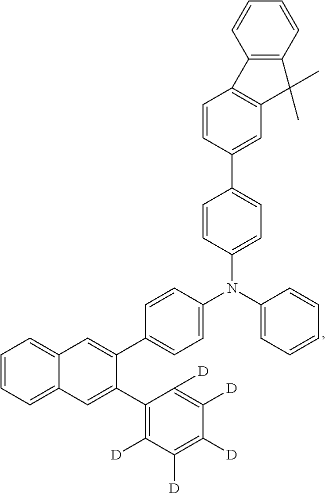 [SMILES: [2H]c1c([2H])c([2H])c(-c2cc3ccccc3cc2-c2ccc(N(c3ccccc3)c3ccc(-c4ccc5c(c4)C(C)(C)c4ccccc4-5)cc3)cc2)c([2H])c1[2H]]

which can be understood as a deuterated form of Compound 130; or

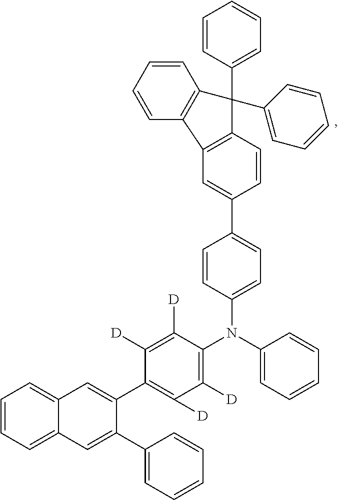 [SMILES: [2H]c1c([2H])c(N(c2ccccc2)c2ccc(-c3ccc4c(c3)-c3ccccc3C4(c3ccccc3)c3ccccc3)cc2)c([2H])c([2H])c1-c1cc2ccccc2cc1-c1ccccc1]

which can be understood as a deuterated form of Compound 136. Numerous other compounds share this characteristic, which are not exhaustively listed or numbered here. To obtain such deuterated compounds, it is only necessary to select deuterium-substituted starting materials during preparation. The preparation process is identical to that used for structurally identical non-deuterated compounds.

In an embodiment of the present disclosure, the first type of aromatic amine compound is a substituted compound A″, where the unsubstituted compound A″ is selected from the group consisting of Compounds 1 to 609. The substituted compound A″ is formed by replacing at least one hydrogen atom on the unsubstituted compound A″ with deuterium.

The present disclosure also provides a method for preparing a first type of aromatic amine compounds, including the following steps:

Adding Compound a and Compound b to a solvent, heating under reflux for 0.5-1.5 h under an inert atmosphere, cooling to blow 80° C., then adding potassium carbonate and tetrakis(triphenylphosphine)palladium(0), and continuing reflux for 8-10 h. Upon completion of the reaction, washing the mixture with water, extracting the organic phase with dichloromethane, drying over anhydrous magnesium sulfate, and concentrating under reduced pressure obtain a crude product. Purifying the crude product by recrystallization from dichloromethane and n-heptane to afford Compound c;

Adding Compound c and Compound d to a solvent, heating under reflux for 1.5-2 hours under an inert atmosphere, adding sodium tert-butoxide, (2-dicyclohexylphosphino-2′,6′-dimethoxybiphenyl), and tris(dibenzylideneacetone)dipalladium(0), and continuing reflux for 8-10 h. Upon completion of the reaction, quenching with water, extracting with dichloromethane and water, drying the organic phase over anhydrous magnesium sulfate, concentrating under reduced pressure, and purifying by column chromatography followed by recrystallization from toluene and n-heptane to obtain the product;
wherein, Compound a is selected from one of

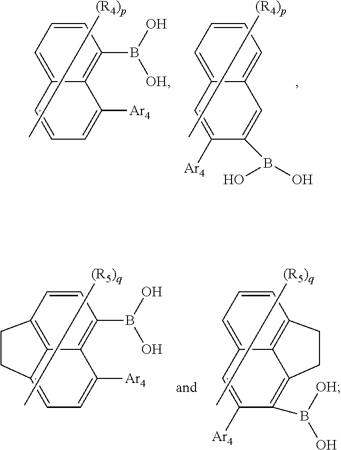

Compound b is selected from

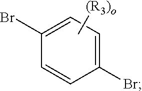

Compound d is selected from

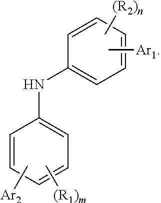

Ar₁, Ar₂, and Ar₄ may be the same or different, each independently representing hydrogen, deuterium, a substituted or unsubstituted alkyl group having 1 to 10 carbon atoms, a substituted or unsubstituted cycloalkyl group having 1 to 10 carbon atoms, a substituted or unsubstituted aryl group having 6 to 30 carbon atoms, a substituted or unsubstituted heteroaryl group having 3 to 30 carbon atoms, or a substituted or unsubstituted cycloalkylnaphthalene. The heteroaryl group contains at least one or more heteroatoms selected from O, S, N, and Si;
R₁-R₅ may be the same or different, each independently representing hydrogen, deuterium, a substituted or unsubstituted alkyl group having 1 to 20 carbon atoms, a substituted or unsubstituted cycloalkyl group having 3 to 10 carbon atoms, a substituted or unsubstituted silyl group, or a substituted or unsubstituted arylsilyl group having 6 to 20 carbon atoms;
when Ar₁, Ar₂, Ar₄, or R₁-R₅ has substituents, the substituents may be the same or different, each independently selected from deuterium, halogen, an alkylsilyl group having 1 to 30 carbon atoms, an arylsilyl group having 6 to 30 carbon atoms, an arylamino group having 6 to 30 carbon atoms, an alkyl group having 1 to 30 carbon atoms, an alkenyl group having 2 to 30 carbon atoms, or an aryl group having 6 to 30 carbon atoms.
m represents an integer of 0-5;
n represents an integer of 0-5;
o represents an integer of 0-4;
p represents an integer of 0-6;
q represents an integer of 0-4;

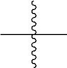

represents a connecting bond.

According to an aspect of the present disclosure, the present disclosure also provides an application of aromatic amine compounds in the preparation of organic electroluminescent devices, particularly an application of the first type of aromatic amine compounds in the preparation of organic electroluminescent devices.

According to an aspect of the present disclosure, the present disclosure also provides an organic electroluminescent device, which comprises an anode, a cathode, and at least one organic layer disposed between the anode and the cathode. The organic layer includes one or more of the first type of aromatic amine compounds.

In an embodiment of the present disclosure, the organic layer includes a hole injection layer, a hole transport layer, an electron barrier layer, an emitting layer, a hole barrier layer, an electron transport layer, and/or an electron injection layer;
wherein, the material for the hole transport layer, electron barrier layer, and/or emitting layer includes one or more of the first type of aromatic amine compounds.

According to an aspect of the present disclosure, a display apparatus is also provided, which includes the electroluminescent device according to the embodiments of the present disclosure.

The first type of aromatic amine compounds provided in the disclosure exhibits the following advantages:
1. By introducing naphthyl groups or cycloalkylnaphthalene structures connected at 1,8- or 2,3-positions in Ar₃, the aromatic amine compounds can increase theses steric hindrance and prevent intermolecular stacking, thereby enhances film-forming properties.
2. When introducing naphthyl groups or cycloalkylnaphthalene structures connected at 1,8- or 2,3-positions in Ar₃, the compounds provide greater tunability for physical properties. This effectively blocks exciton recombination at interfaces and further improves charge mobility.
3. When introducing cycloalkylnaphthalene structures in Ar₃, in addition to maintaining the same characteristics as naphthyl-containing analogs, it increases the solubility of the aromatic amine compounds. This facilitates cleaning of evaporation masks, moderately optimizes sublimation temperatures, and enhances device stability.
4. By adjusting substituents on the aromatic amine compounds, a series of materials with distinct properties can be obtained. For example, incorporating groups such as dibenzofuran, carbazole, or fluorene ensures high T1 (first triplet state) energy, effectively blocks excitons, and improves overall performance.
5. These aromatic amine compounds exhibit strong hole transport capabilities, appropriate HOMO energy levels and T1 values, and high glass transition temperatures (Tg). With high hole mobilities, they can serve as hole-transport materials, electron-barrier materials, or light-emitting layer materials. They enhance device lifetimes and current efficiencies, and can significantly reduce device voltages.

In an embodiment of the present disclosure, the first type of aromatic amine compounds of the present disclosure is suitable for use in light-emitting elements, display panels, and electronic devices, particularly in organic electroluminescent devices. The electronic device of the present disclosure comprises at least one layer containing an organic compound, and may also include layers composed of inorganic materials or entirely of inorganic materials. The electronic device is preferably selected from organic electroluminescent devices (OLEDs), organic integrated circuits (O-ICs), organic field-effect transistors (O-FETs), organic thin-film transistors (O-TFTs), organic light-emitting transistors (O-LETs), organic solar cells (O-SCs), organic dye-sensitized solar cells (O-DSSCs), organic optical detectors, organic photoreceptors, organic field-quenching devices (O-FQDs), light-emitting electrochemical cells (LECs), organic laser diodes (O-lasers), and organic plasmon emission devices. Most preferably, the electronic device is an organic electroluminescent device (OLED).

The first type of aromatic amine compounds of the present disclosure can be synthesized using representative reactions such as the Buchwald-Hartwig coupling reaction, Suzuki coupling reaction, or Heck coupling reaction.

In an embodiment of the present disclosure, a second type of aromatic amine compounds is provided. The structural formula of the second type of aromatic amine compounds is shown in Chemical Formula II or Chemical Formula III:

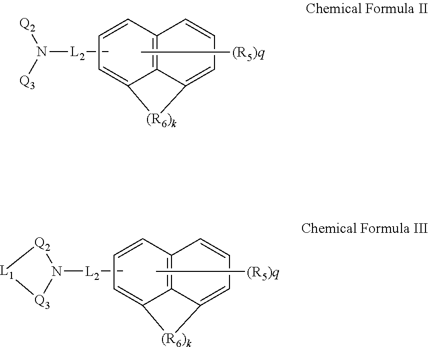

wherein, L₂ is selected from a single bond, a substituted or unsubstituted aryl group having 6 to 30 carbon atoms, or a substituted or unsubstituted heteroaryl group having 3 to 30 carbon atoms.

The cycloalkylnaphthalene introduced in the second type of aromatic amine compounds exhibits strong electron-donating capabilities (increasing electron cloud density), which enhances hole mobility. Compared to naphthalene, its increased molecular weight improves thermal stability (e.g., higher glass transition temperature, Tg), ensuring better device lifetime. Additionally, the cycloalkylnaphthalene increases molecular weight (while also improving material solubility, facilitating the cleaning of evaporation masks) without significantly affecting sublimation temperatures, making it favorable for evaporation processes. The second type of aromatic amine compound enhances material anisotropy through its twisted structure and steric hindrance, effectively increasing lateral steric resistance, thereby improving device crosstalk and lateral leakage issues. Furthermore, by introducing deuterium (D) or stable functional groups, a series of high-performance organic materials can be synthesized, suitable for use as hole-transport materials.

Extensive experimental studies by the inventors reveal that, compared to the first type of aromatic amine compounds containing structural formulas P1-P4, the second type exhibits significantly faster mobility, leading to highly effective improvements in device performance. Moreover, the second type of aromatic amine compound has deeper HOMO energy levels, enabling its application in electron barrier layers. This facilitates faster hole injection from the hole transport layer to the light-emitting functional layer and addresses the slow mobility issue of auxiliary emitting layers (particularly in blue devices) observed in prior technologies.

In an example, R₅ is selected from hydrogen, deuterium, or an aryl group having 6 to 12 carbon atoms.

In an embodiment of the present disclosure,

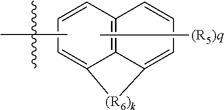

is selected from substituted or unsubstituted W6. Unsubstituted W6 is represented by

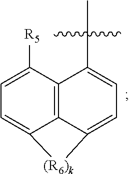

substituted W6 refers to the naphthalene ring of unsubstituted W6 in which one or more hydrogens are replaced by deuterium.

In another embodiment of the present disclosure,

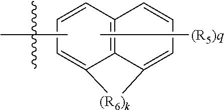

is selected from substituted or unsubstituted W6. Unsubstituted W6 is represented by

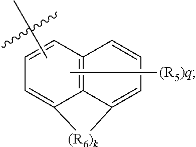

substituted W6 refers to the naphthalene ring of unsubstituted W6 in which one or more hydrogens are replaced by deuterium.

In an embodiment of the second type of aromatic amine compounds, L₂ is selected from a single bond, substituted or unsubstituted phenylene, substituted or unsubstituted biphenylene, or substituted or unsubstituted terphenylene.

In an embodiment of the second type of aromatic amine compounds, when L₂ is substituted with at least one substituent, the substituent(s) on L₂ are selected from deuterium, aryl groups having 6 to 12 carbon atoms, heteroaryl groups having 3 to 12 carbon atoms, alkyl groups having 1 to 4 carbon atoms; when the number of substituents on L₂ is plural, any two substituents are the same or different. Specifically, the substituent(s) on L₂ are selected from deuterium or aryl groups having 6 to 12 carbon atoms.

In an example, when the L₂ is substituted with substituents, the substituents on L₂ are deuterium. that is at least one hydrogen on L₂ is replaced by deuterium, for example, all hydrogens on L₂ are replaced by deuterium.

In an embodiment of the second type of aromatic amine compounds, unsubstituted L₂ is selected from the group consisting of the following structures:

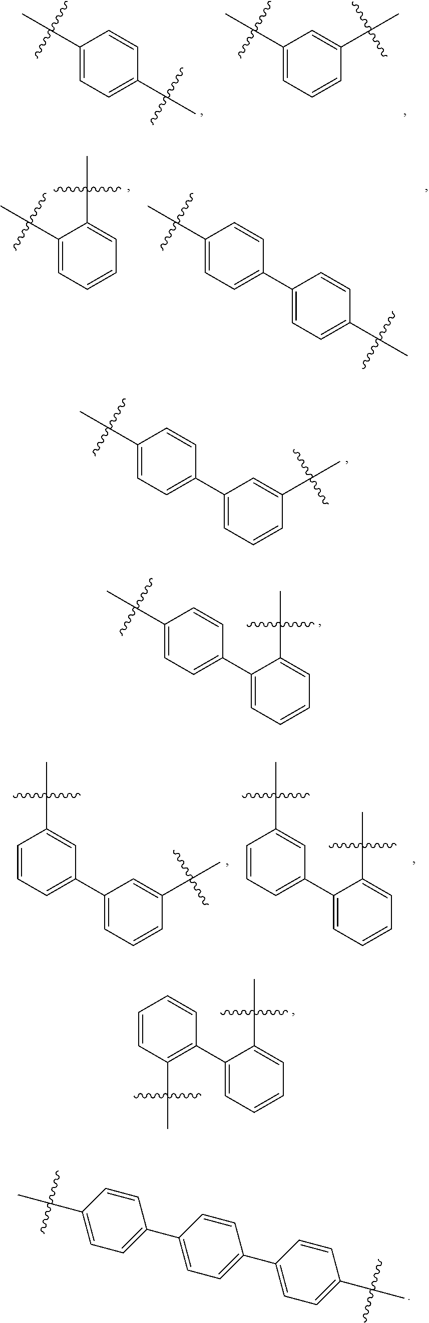

In an embodiment of the second type of aromatic amine compounds, Q₂ is selected from

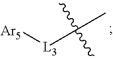

Q₃ is selected from

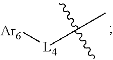

Ar₅ and Ar₆ may be the same or different, and each is independently selected from a substituted or unsubstituted group W1. The unsubstituted group W1 is selected from:

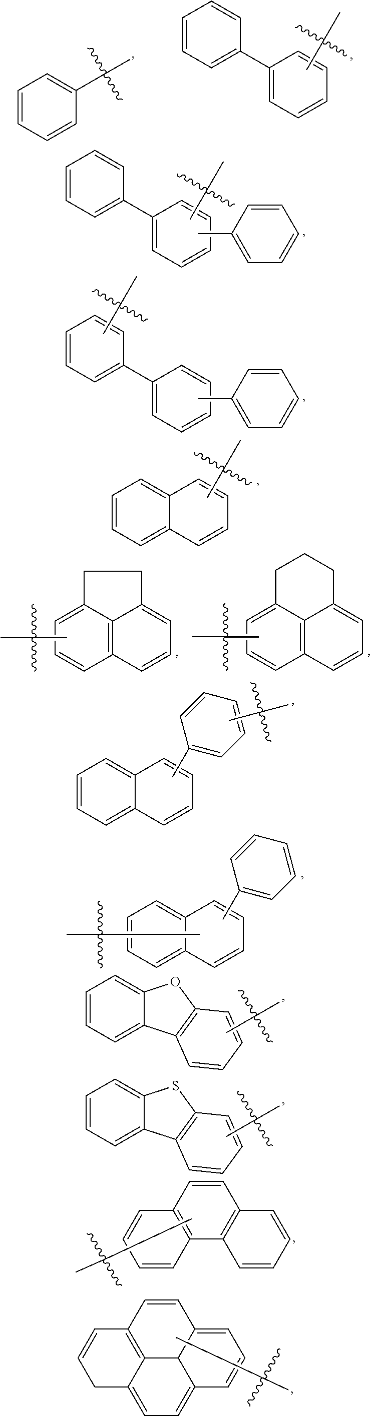

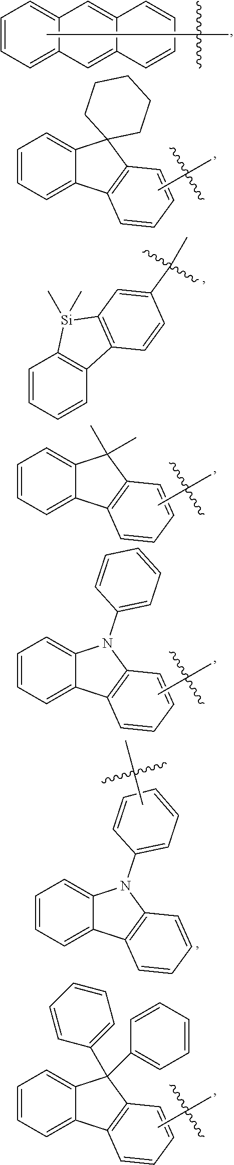

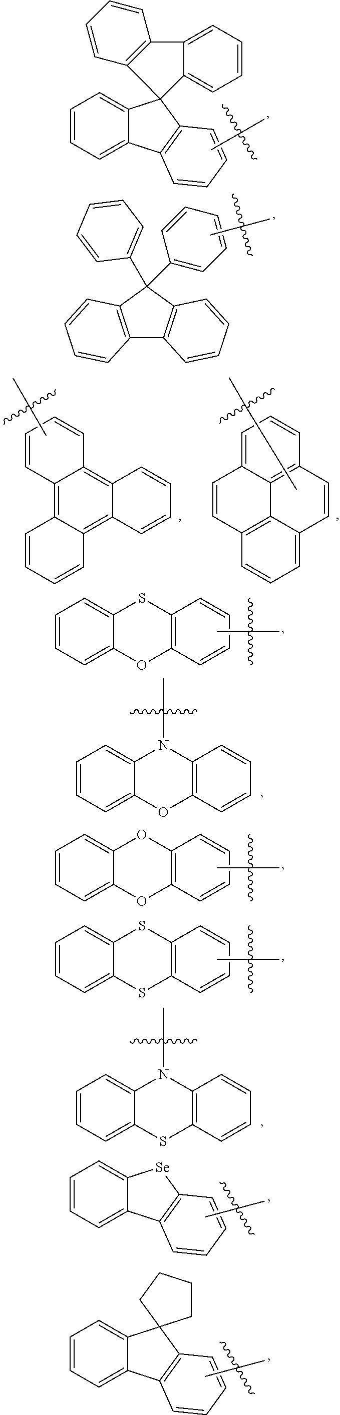

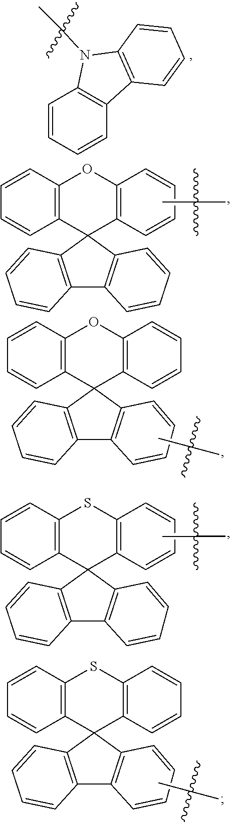

wherein, the substituted group W1 is formed by replacing one or more hydrogens on the unsubstituted group W1 with substituents selected from deuterium, fluorine, chlorine, bromine, an alkyl group having 1 to 6 carbon atoms, a cycloalkyl group having 5 to 10 carbon atoms, an aryl group having 6 to 20 carbon atoms, or a heteroaryl group having 3 to 20 carbon atoms. When multiple substituents are present on the substituted group W1, any two substituents may be the same or different.

In an example, the substituted group W1 is formed by replacing at least one hydrogen on the unsubstituted group W1 with deuterium. For instance, the substituted group W1 may be formed by replacing all hydrogens on the unsubstituted group W1 with deuterium.

In an embodiment of the second type of aromatic amine compounds, L₃ and L₄ may be the same or different, and each is independently selected from a single bond or a substituted or unsubstituted group W2. The unsubstituted group W2 is selected from:

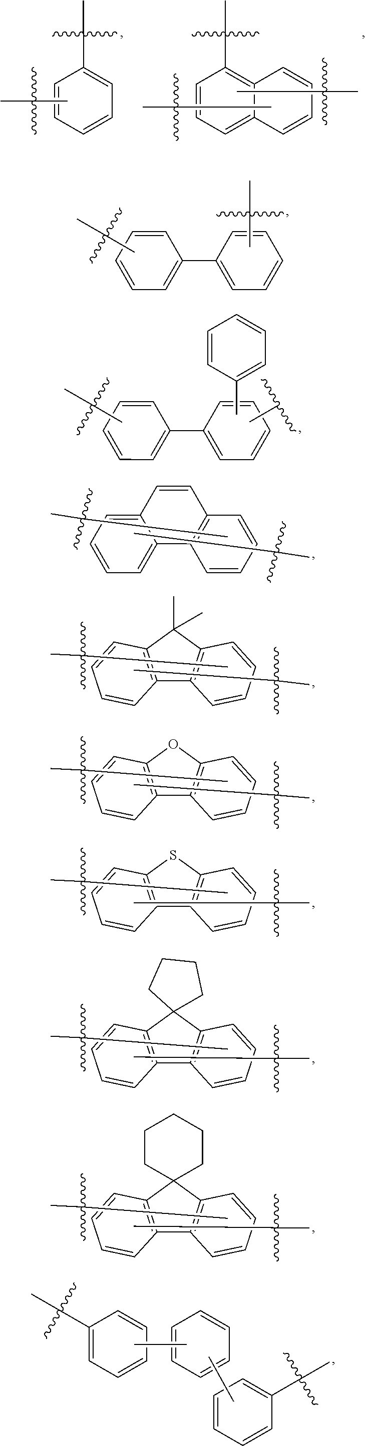

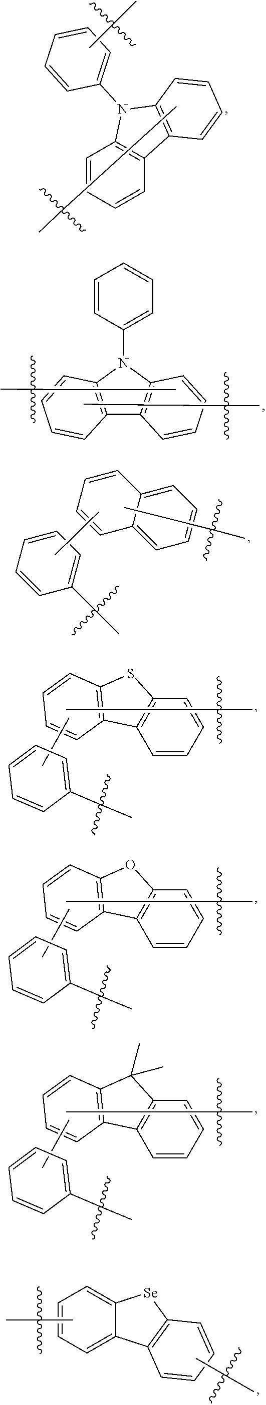

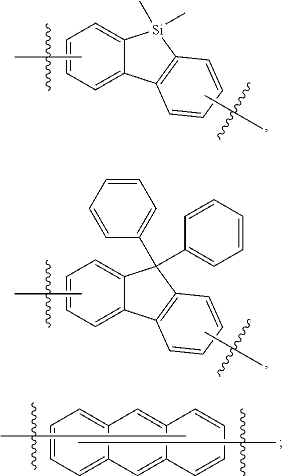

wherein, the substituted group W2 is formed by replacing one or more hydrogens on the unsubstituted group W2 with substituents selected from deuterium, fluorine, chlorine, bromine, an alkyl group having 1 to 6 carbon atoms, a cycloalkyl group having 5 to 10 carbon atoms, an aryl group having 6 to 20 carbon atoms, or a heteroaryl group having 3 to 20 carbon atoms; when the number of substituents on the substituted group W2 is plural, any two substituents may be the same or different.

In an example, the substituted group W2 is formed by replacing at least one hydrogen on the unsubstituted group W2 with deuterium. For instance, the substituted group W2 may be formed by replacing all hydrogens on the unsubstituted group W2 with deuterium.

In an embodiment of the second type of aromatic amine compounds,

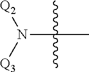

is selected from a substituted or unsubstituted group W3. The unsubstituted group W3 is selected from Formulas a1 to a296:

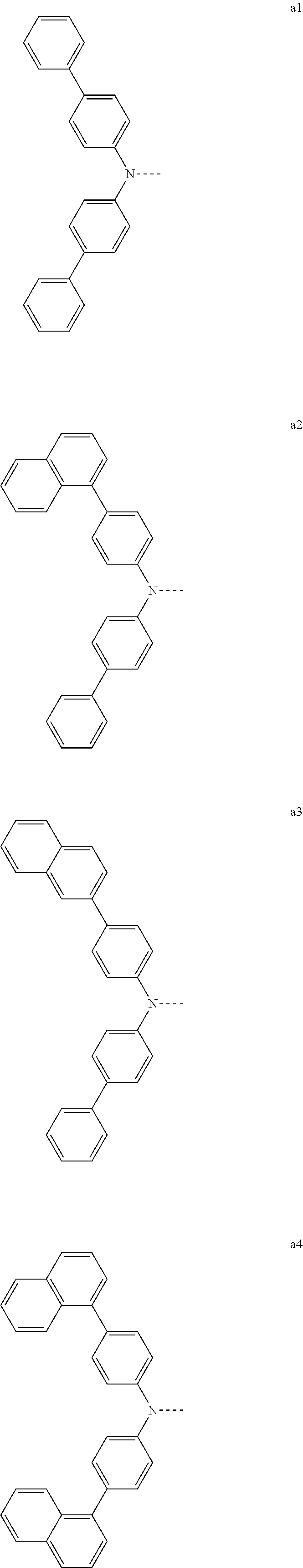

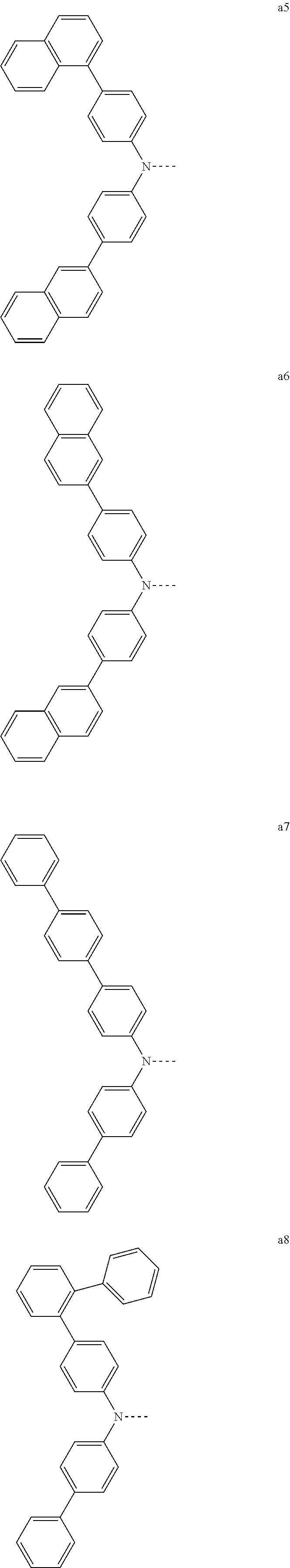

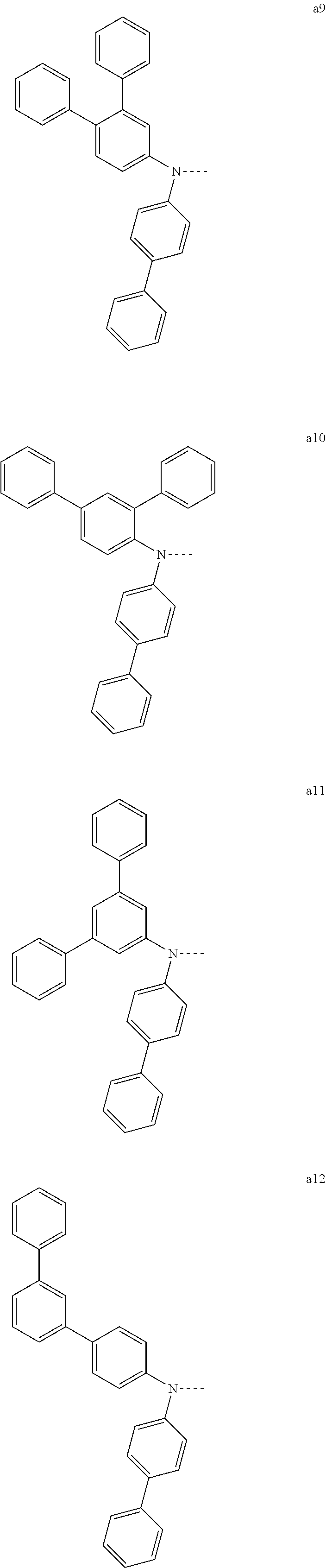

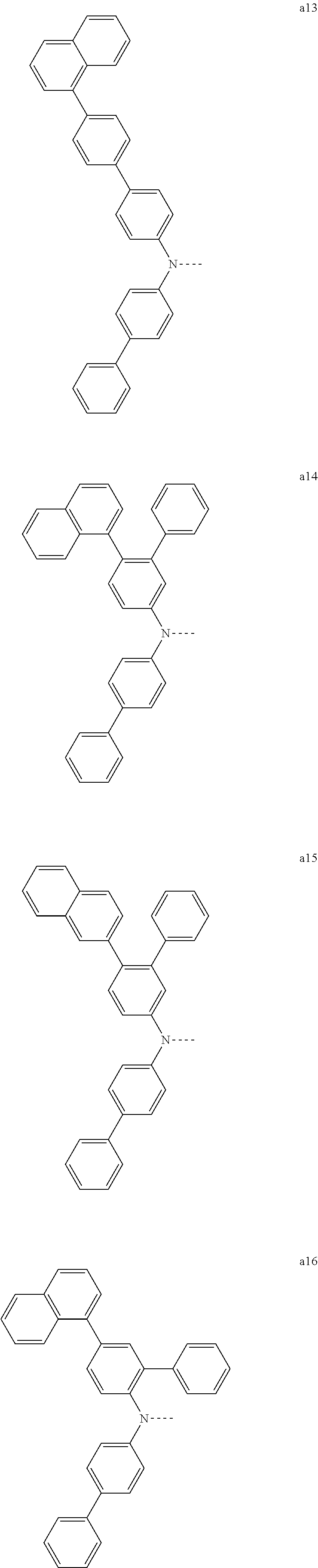

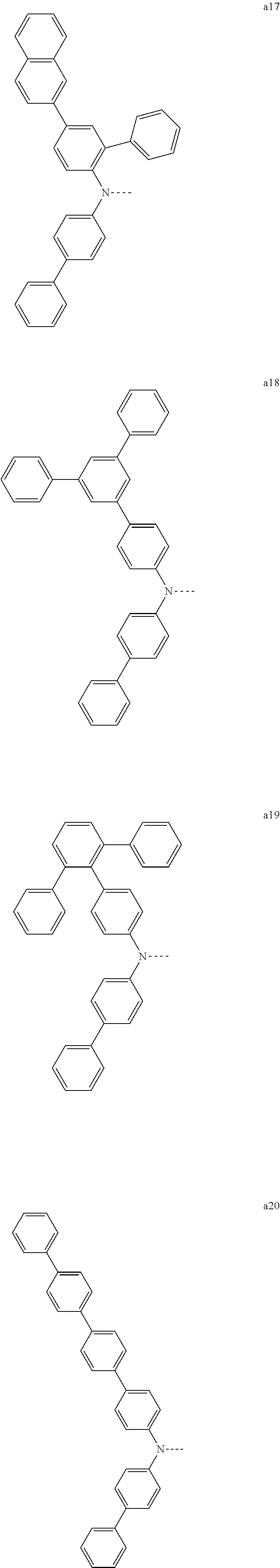

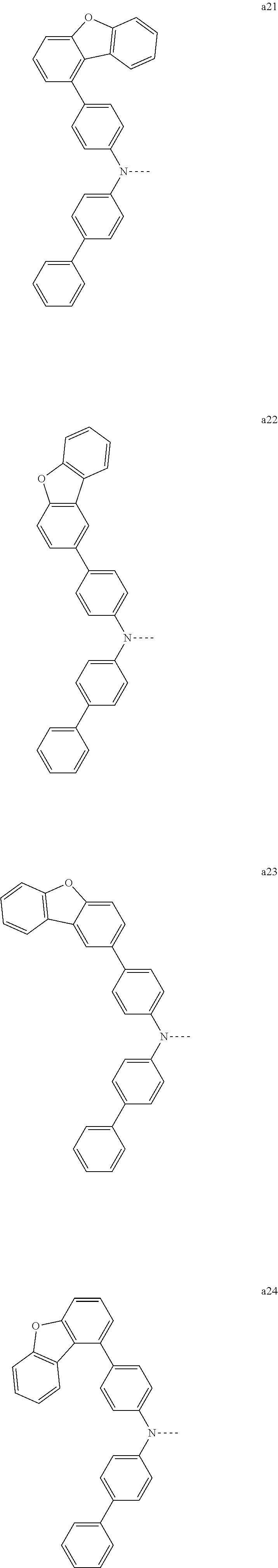

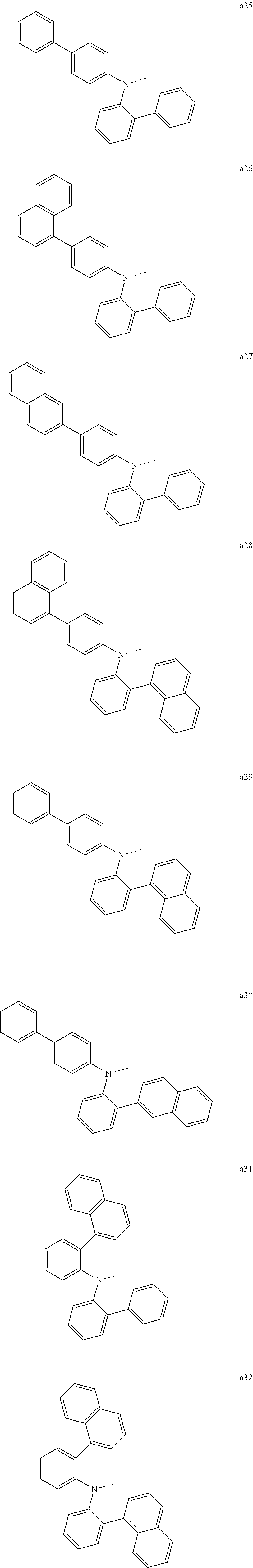

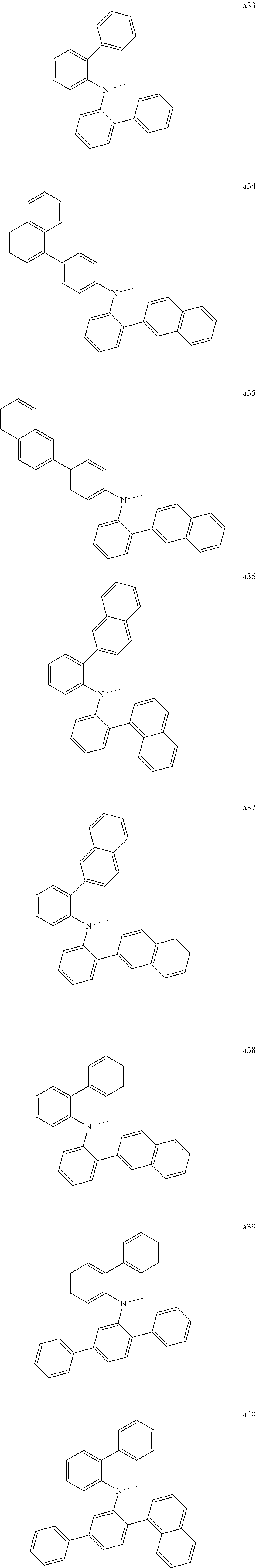

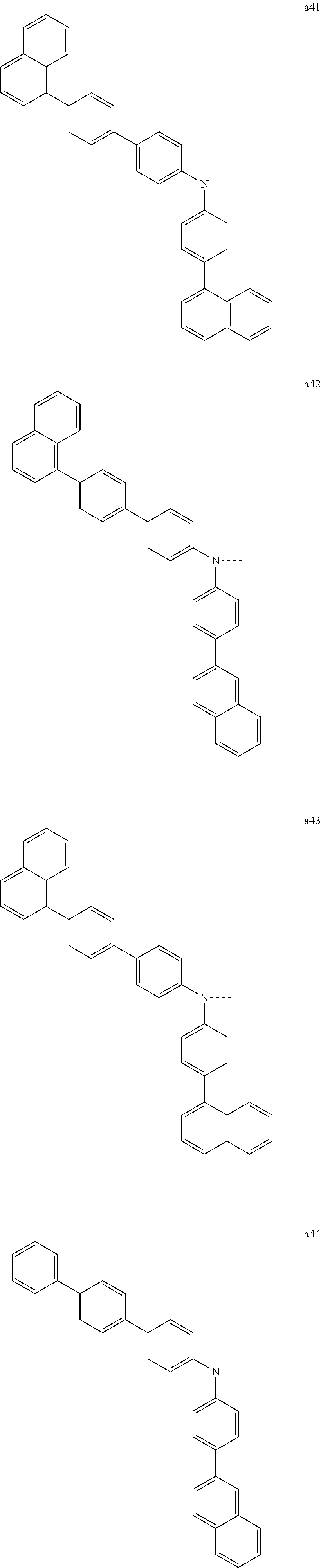

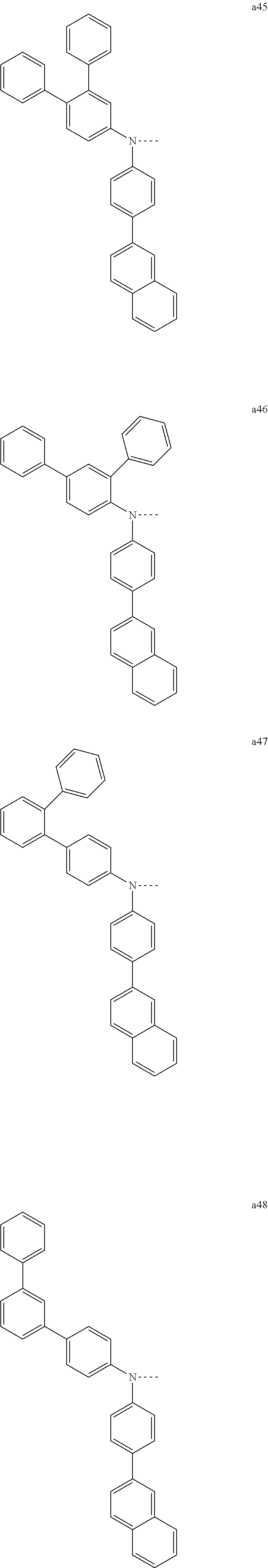

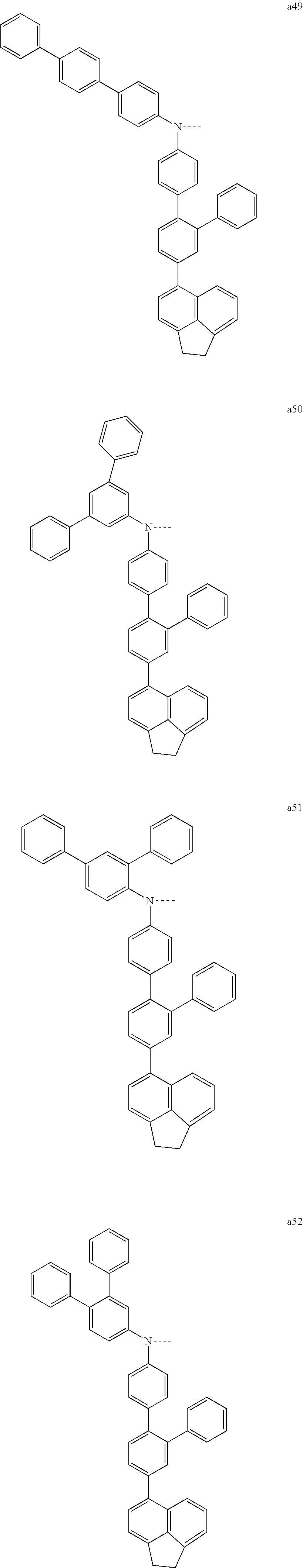

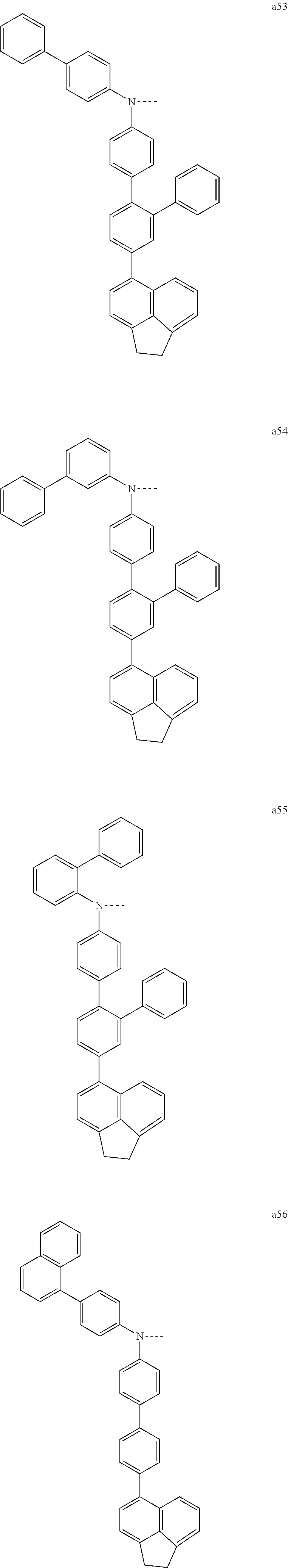

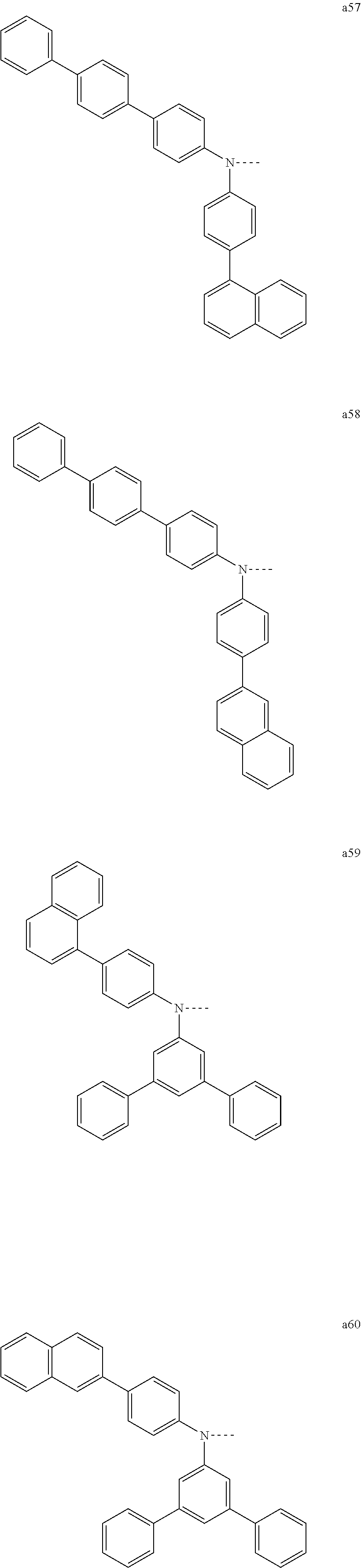

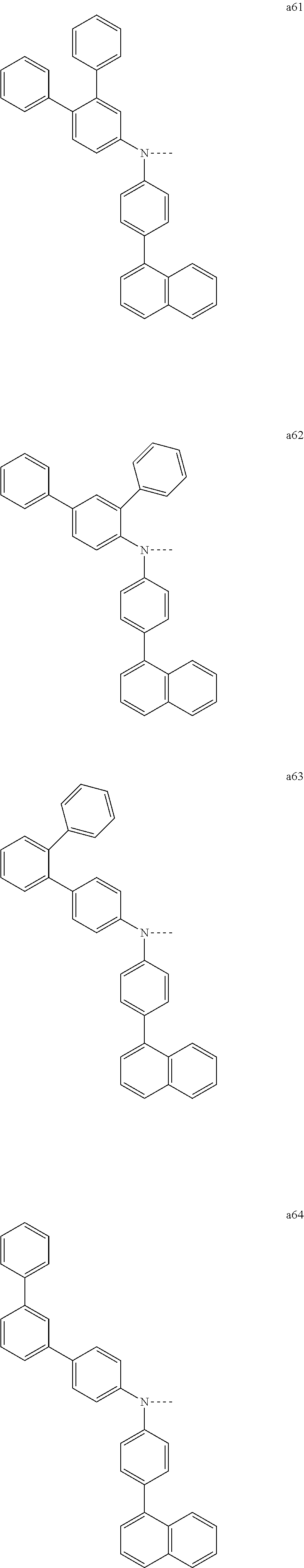

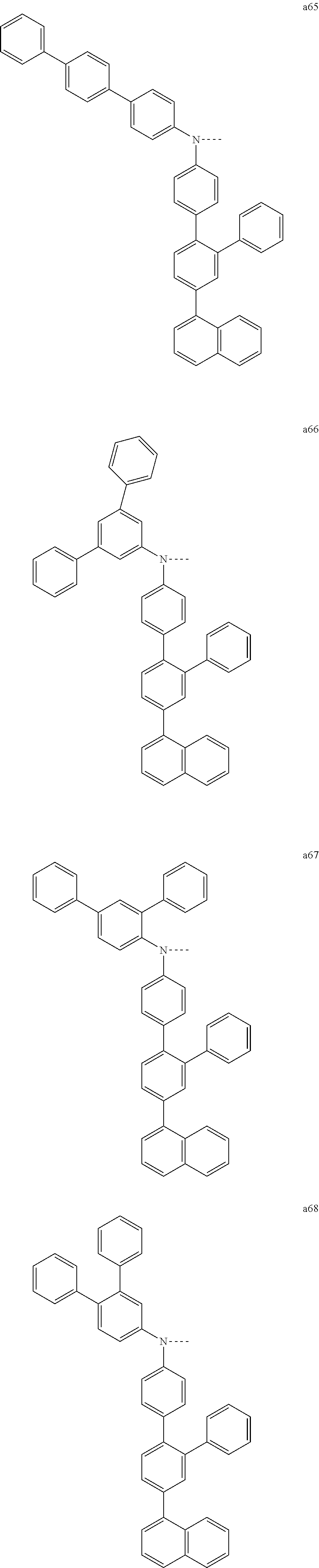

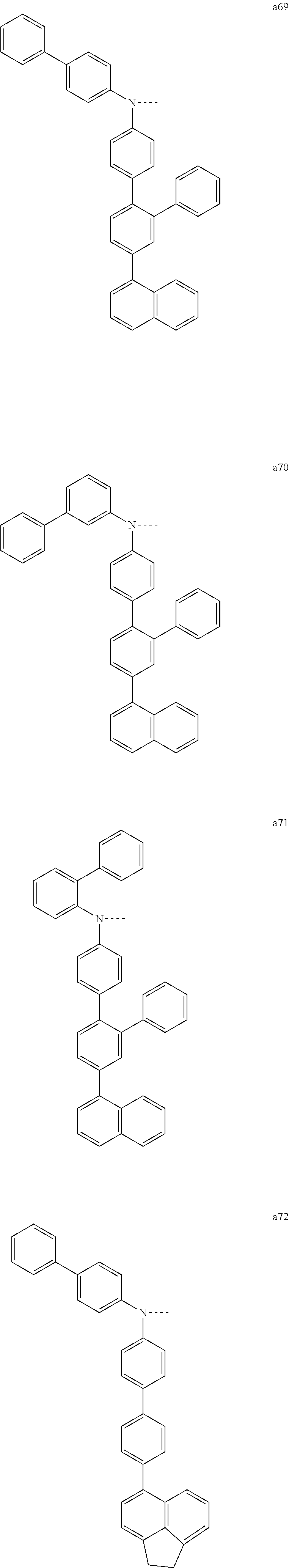

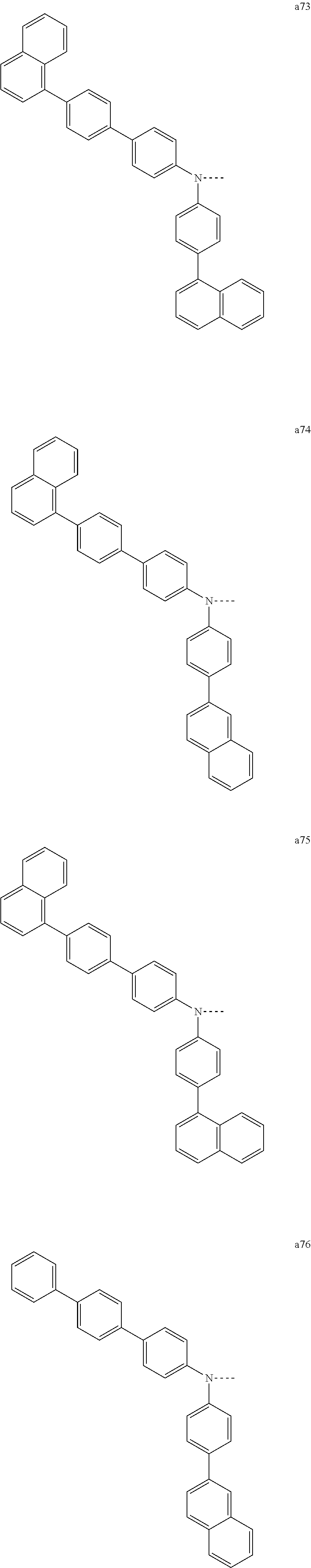

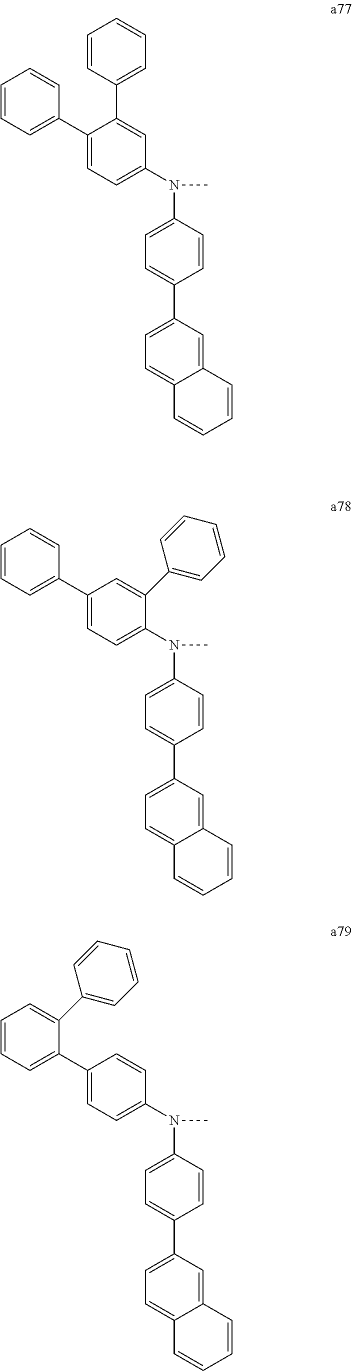

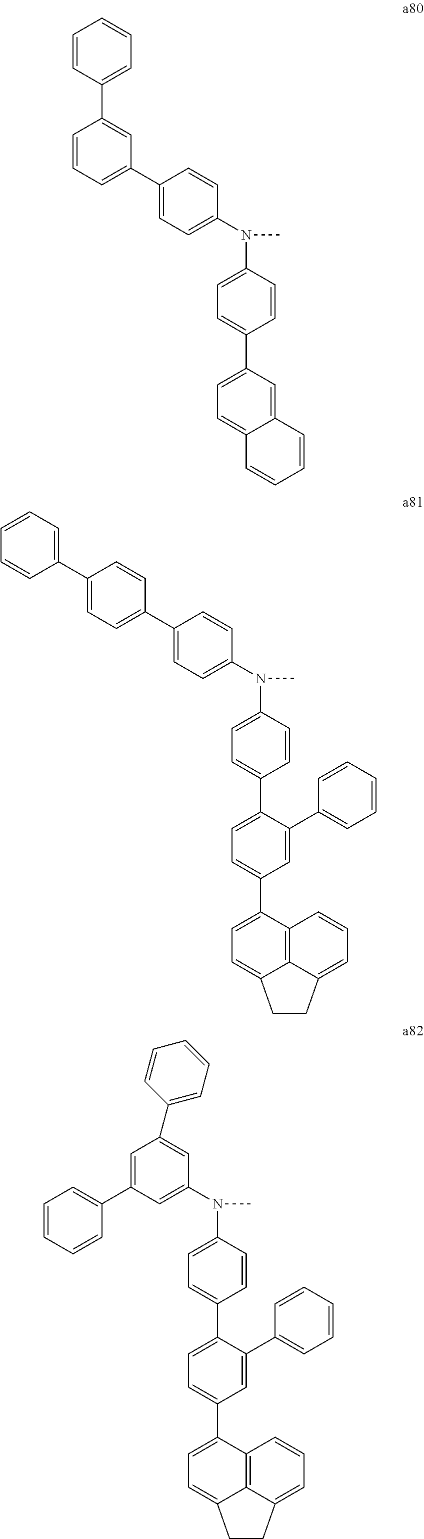

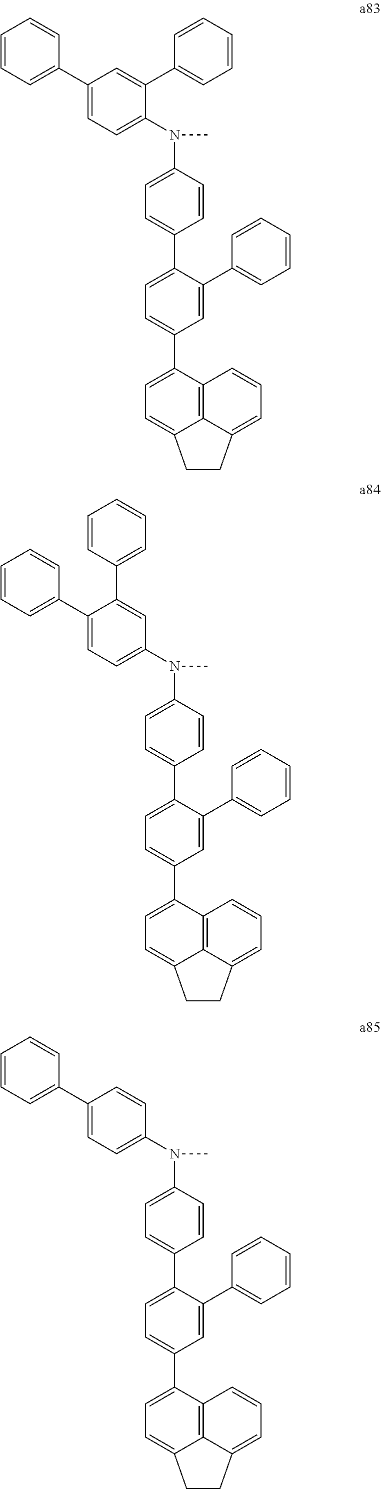

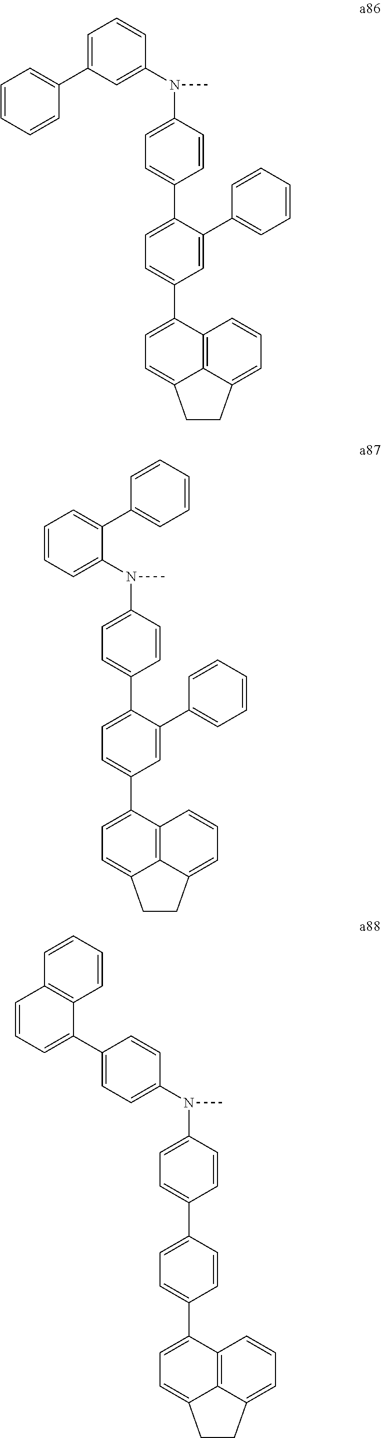

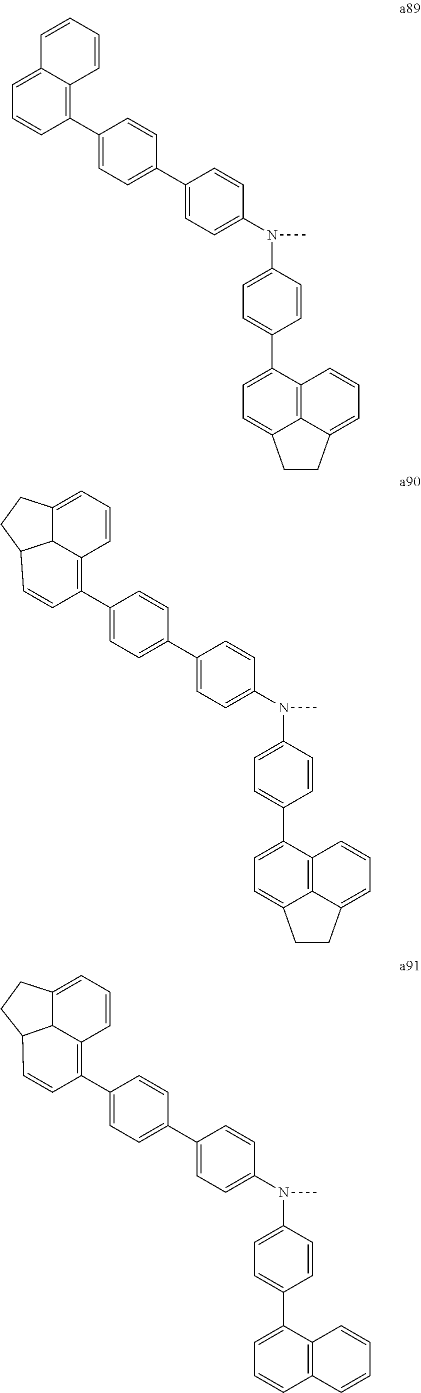

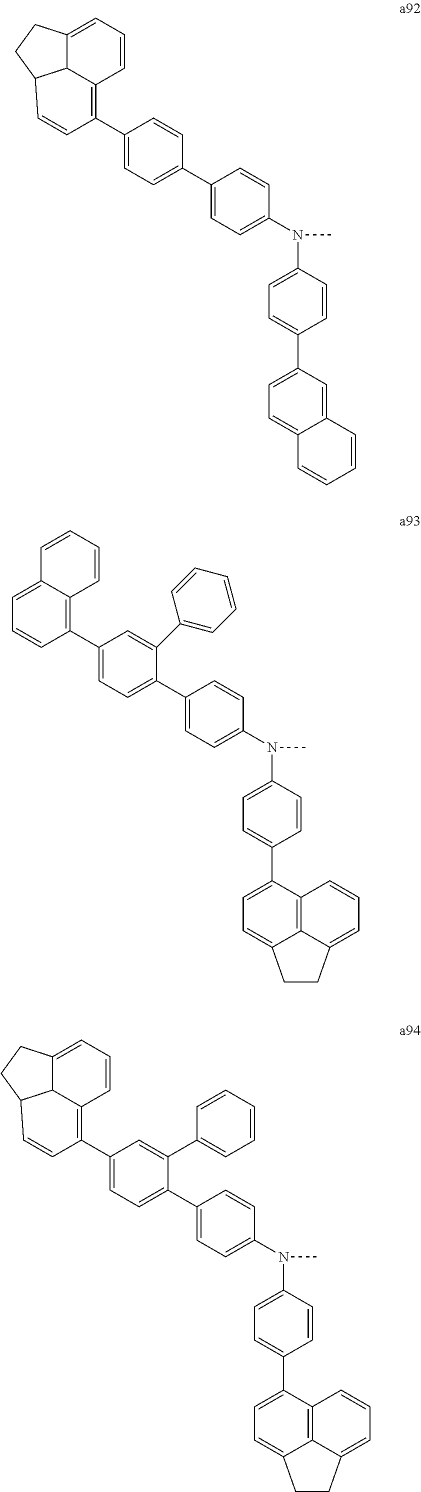

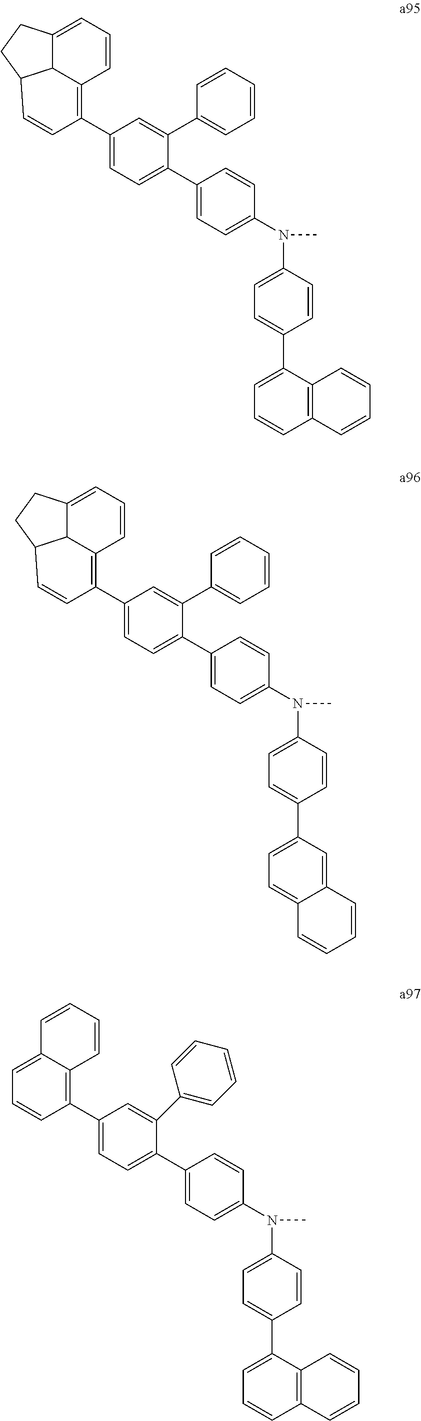

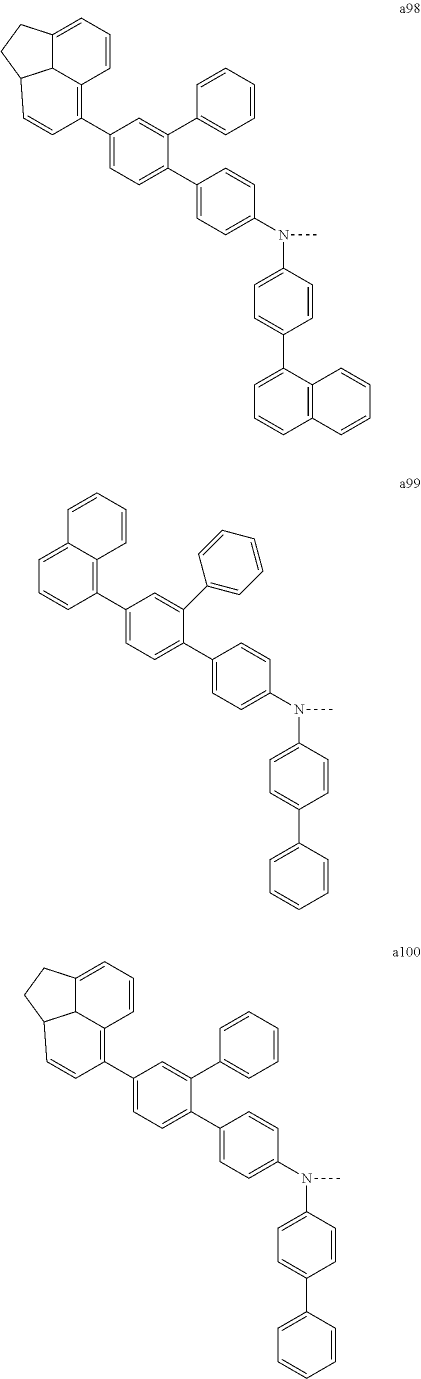

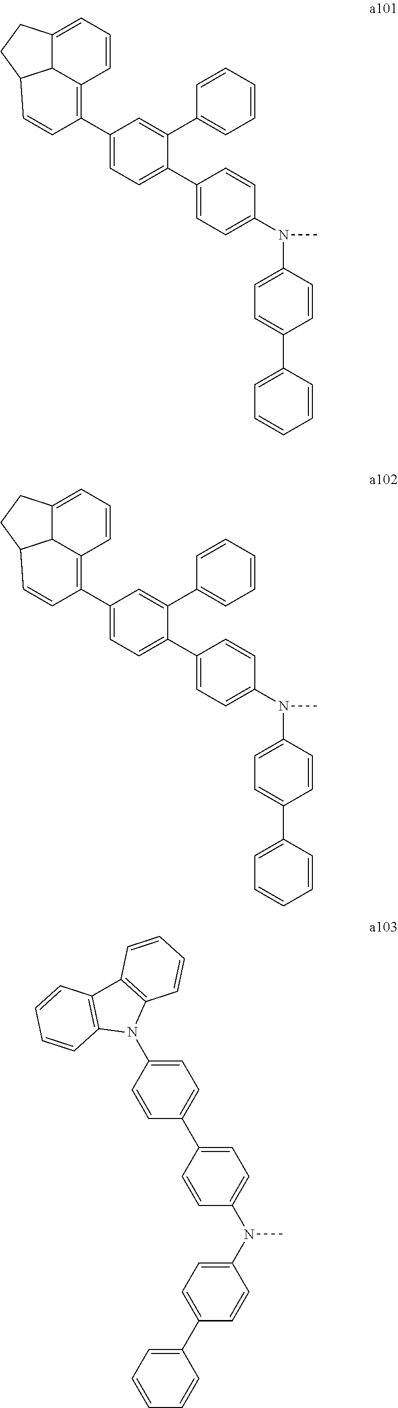

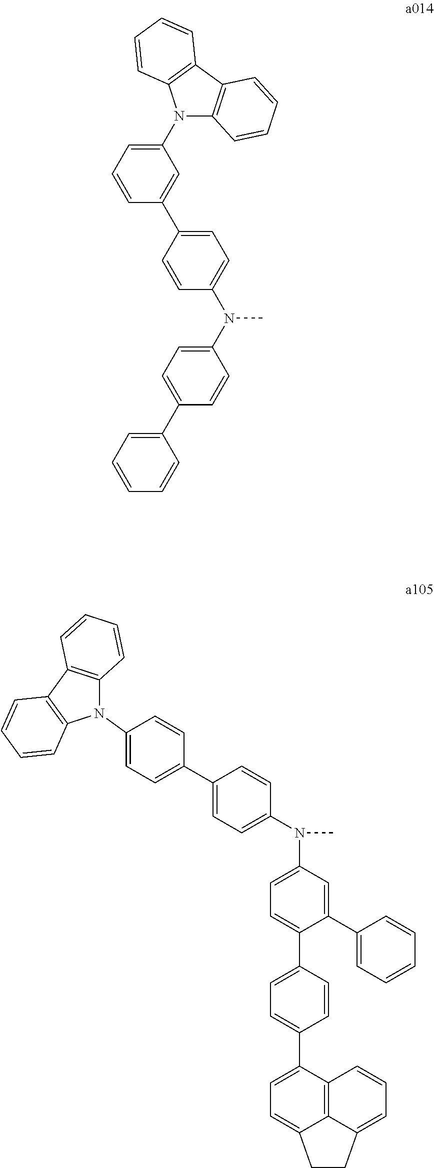

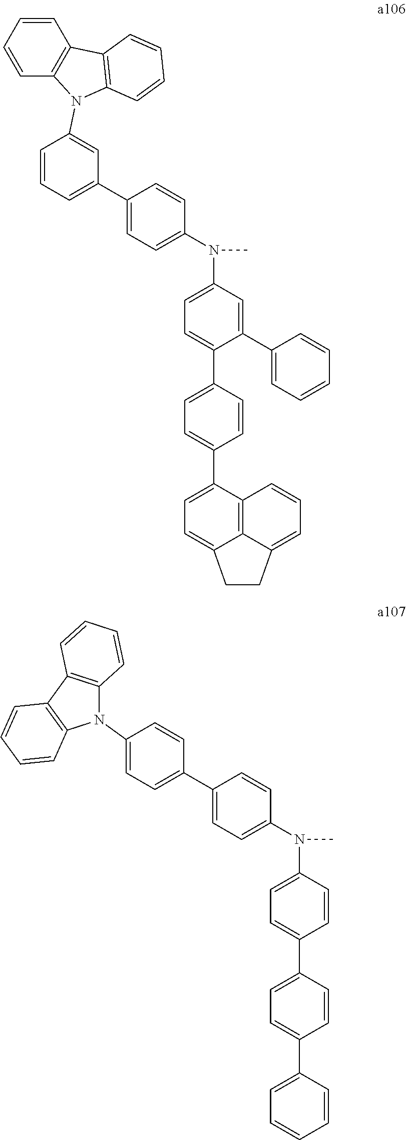

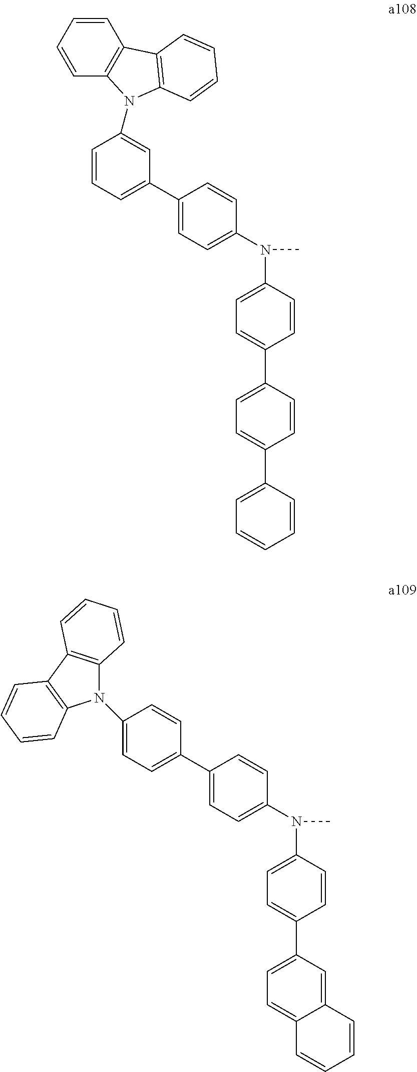

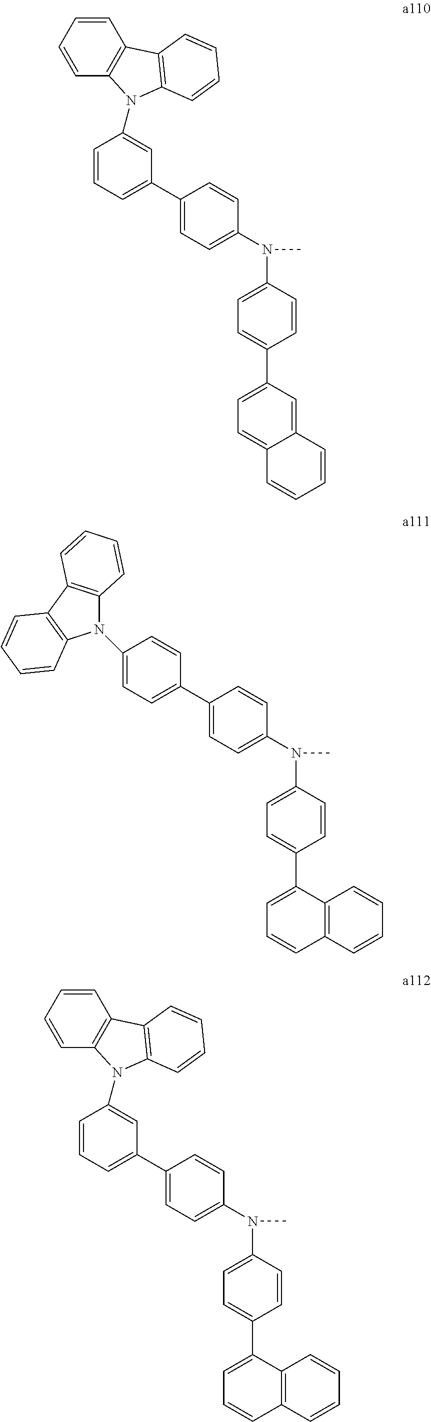

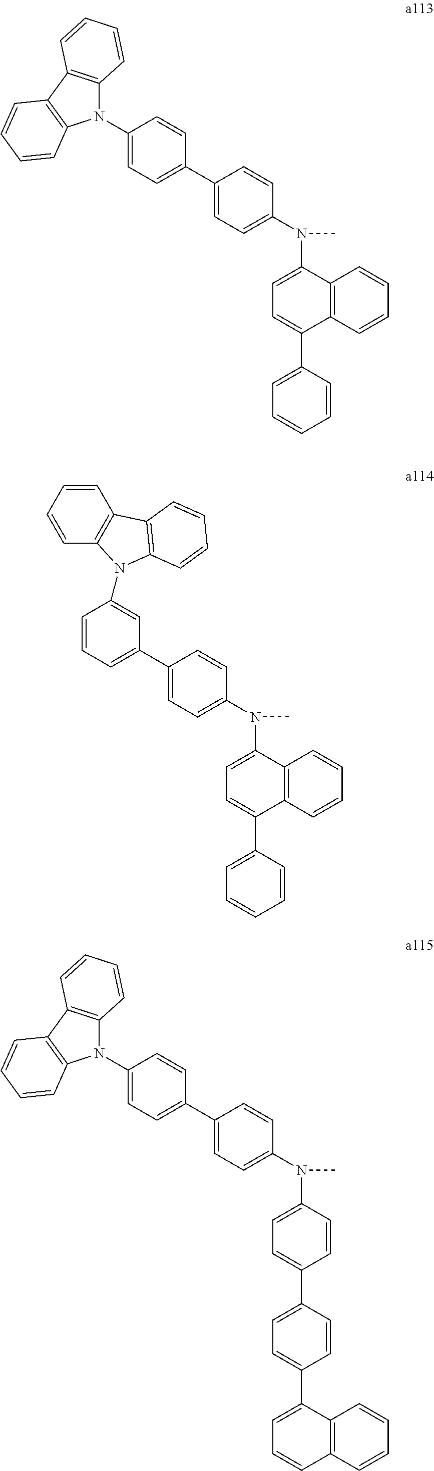

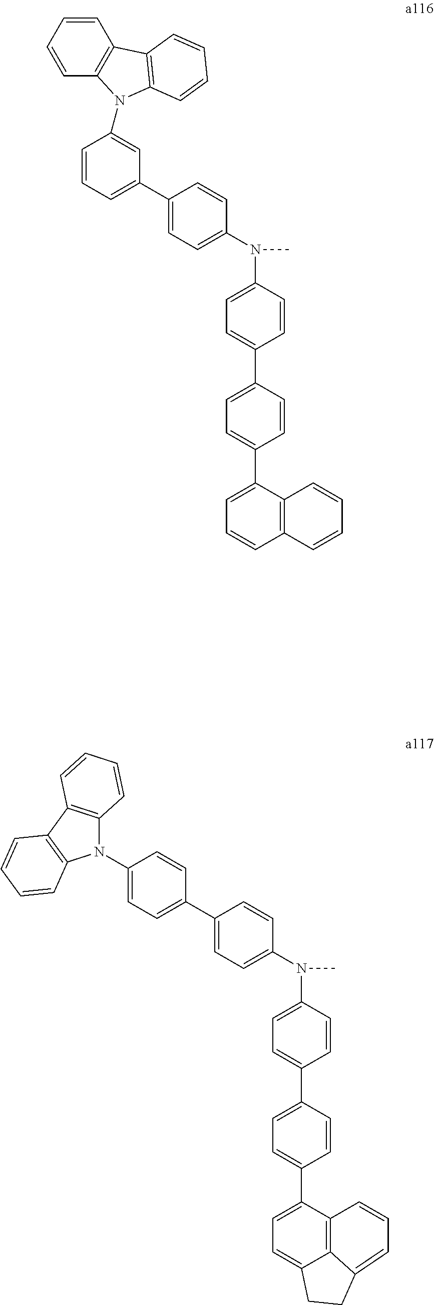

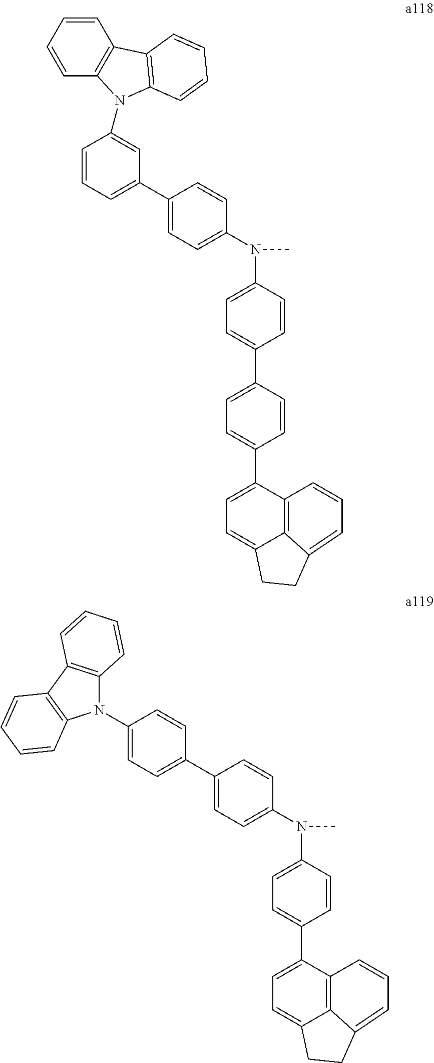

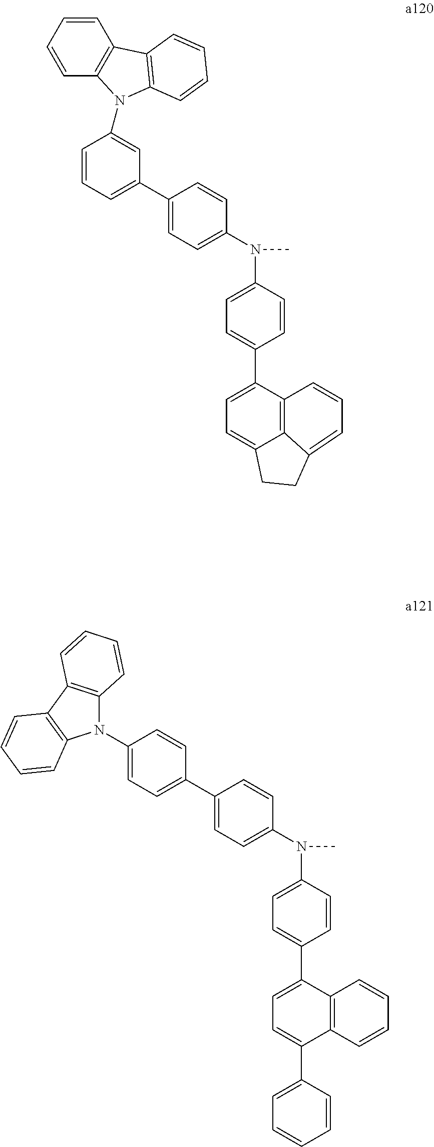

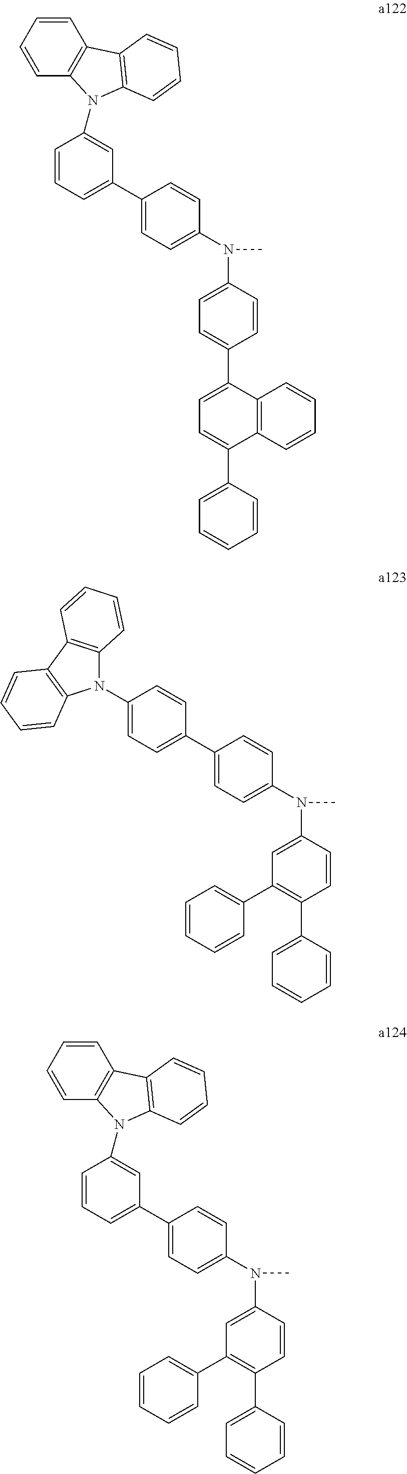

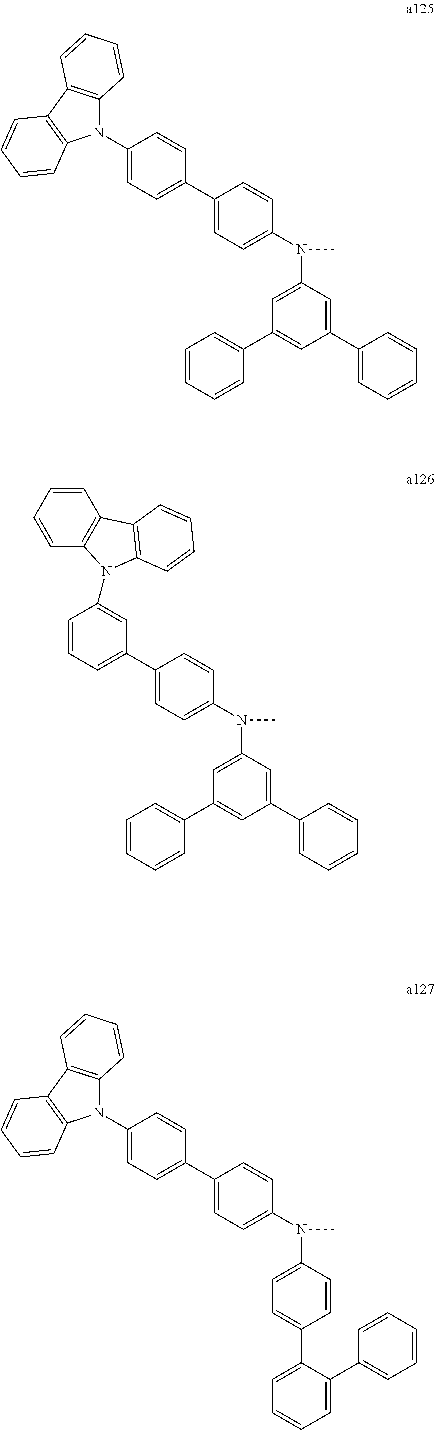

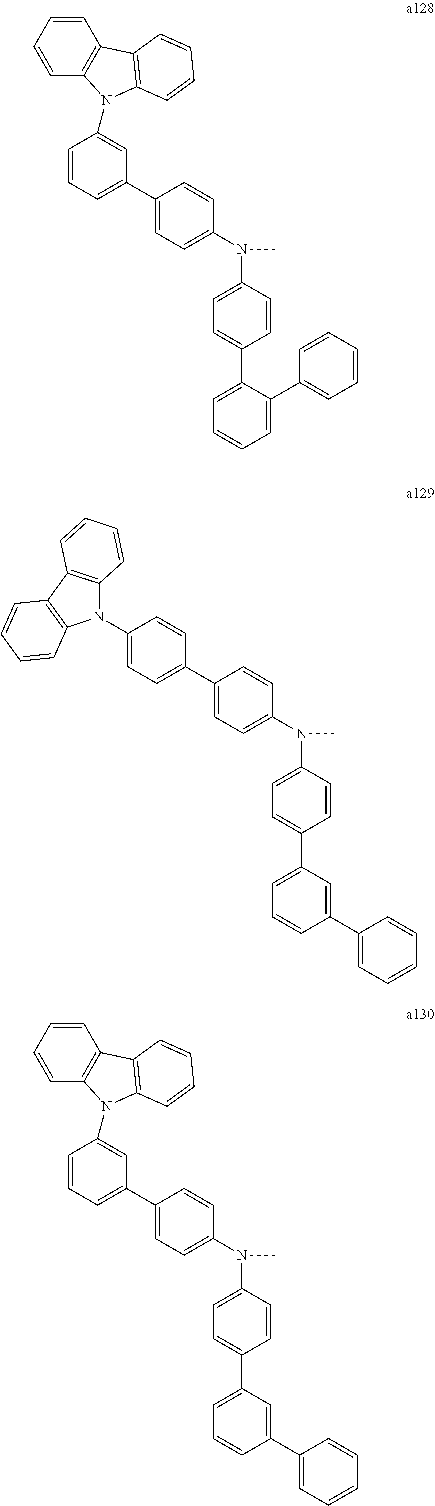

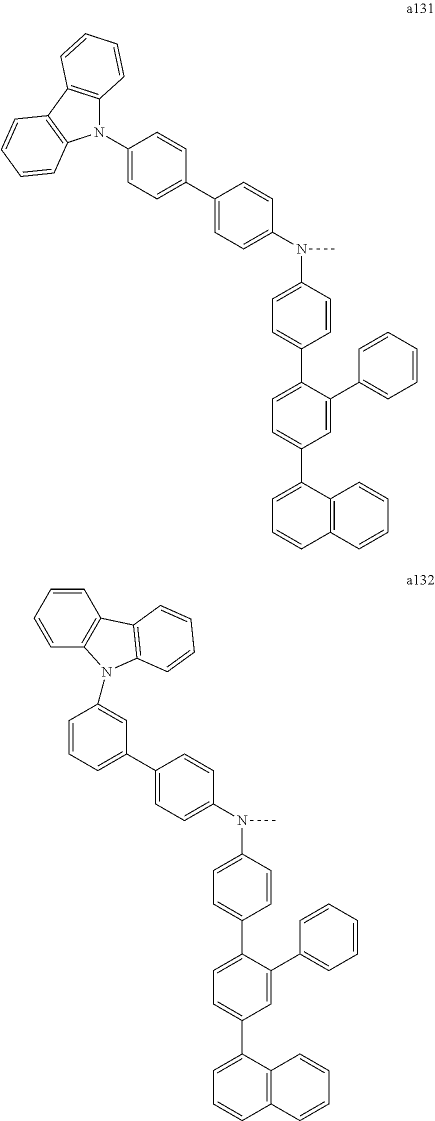

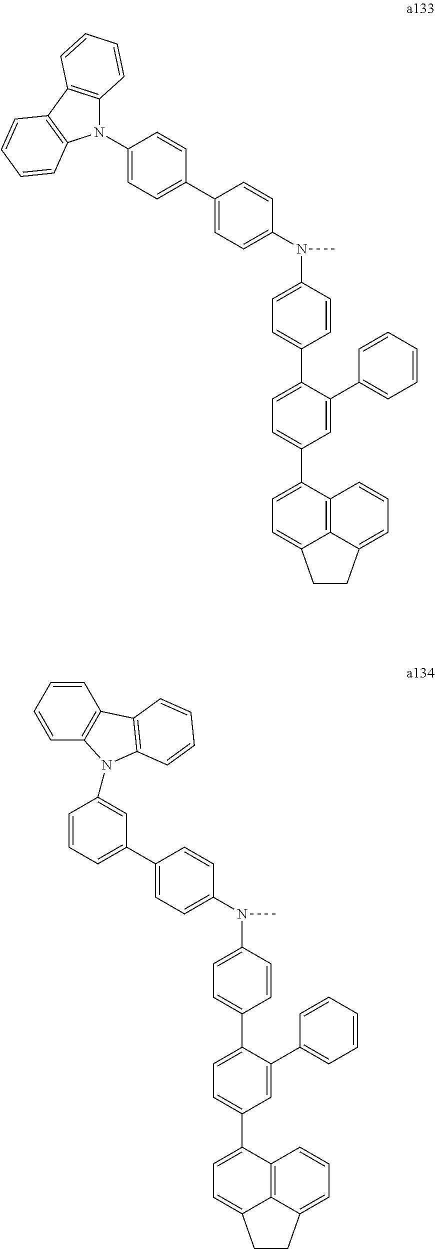

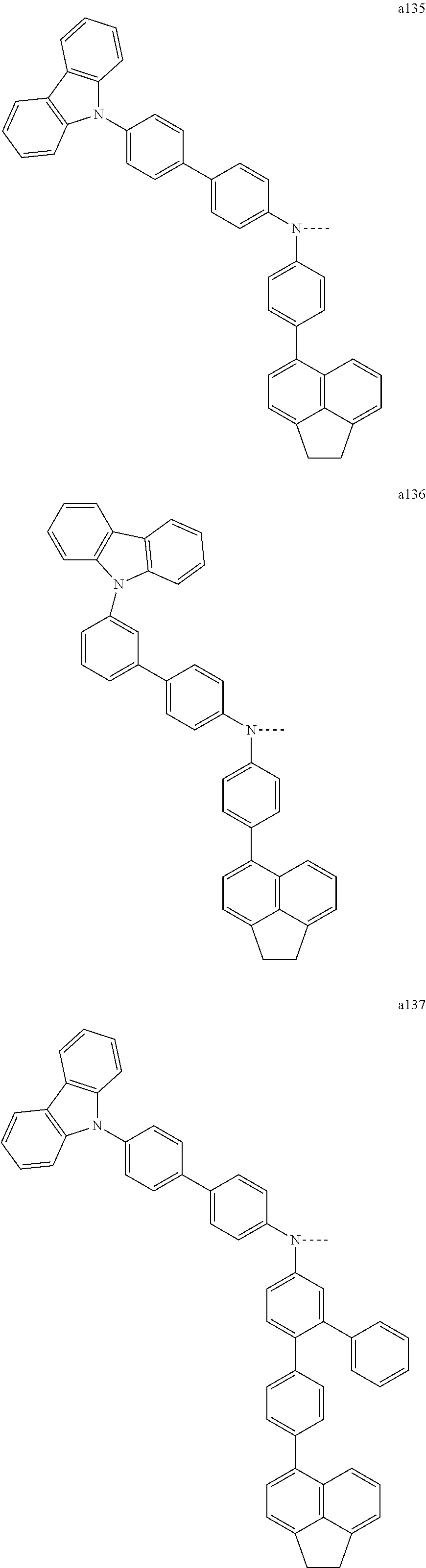

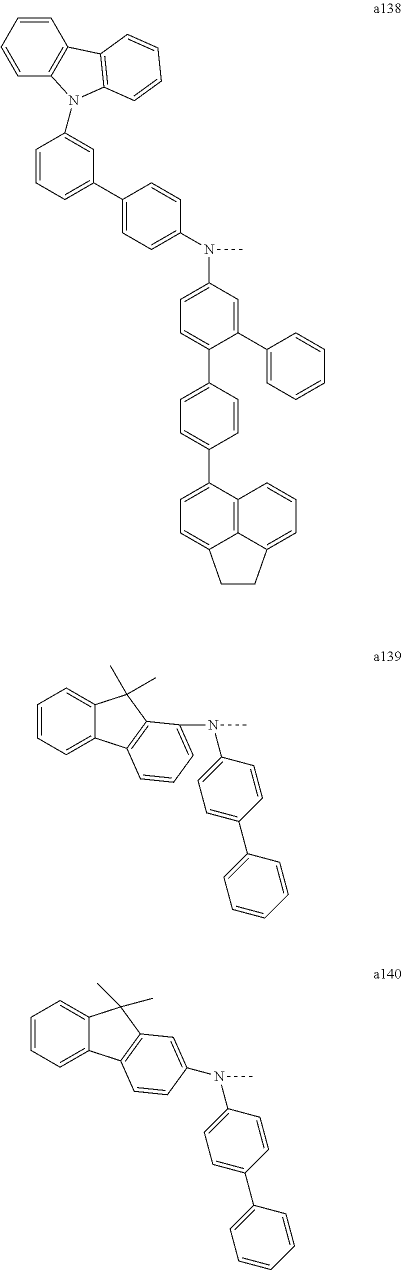

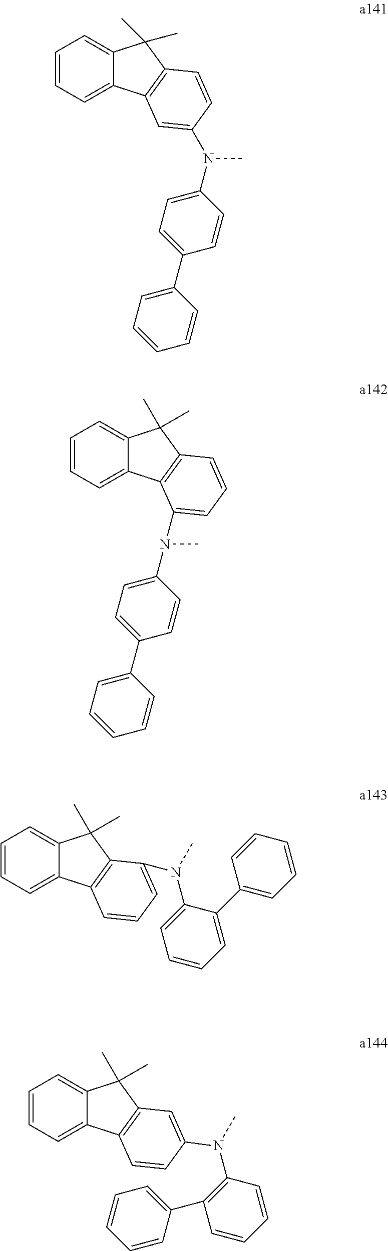

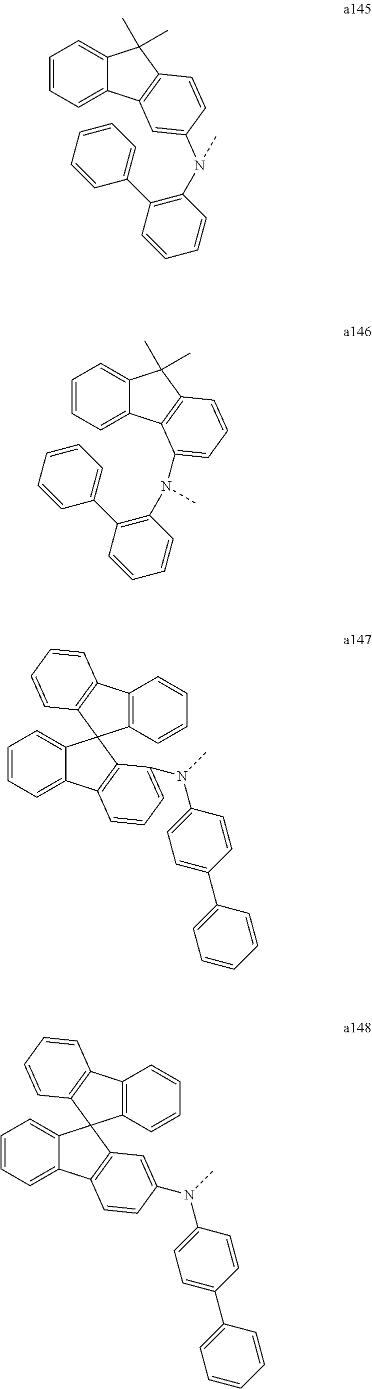

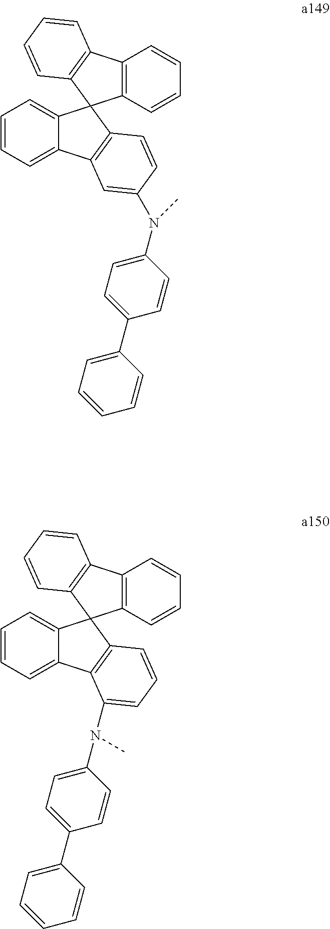

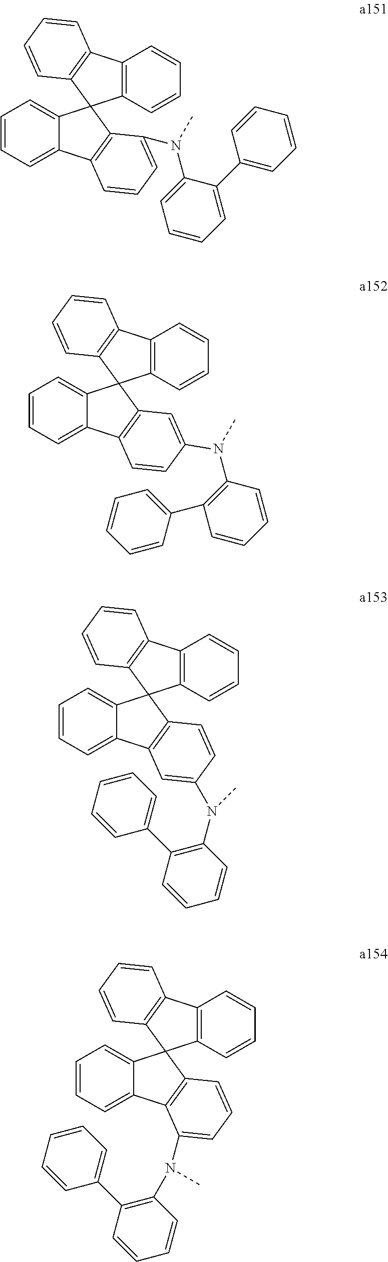

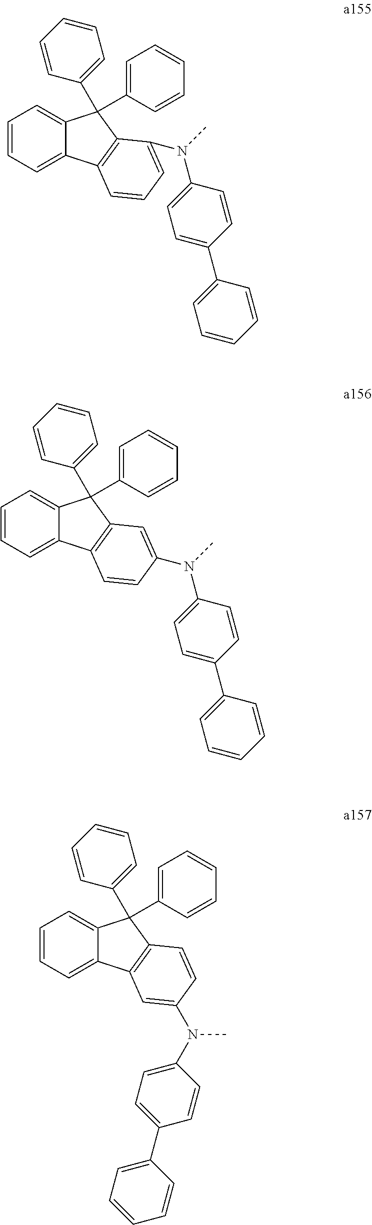

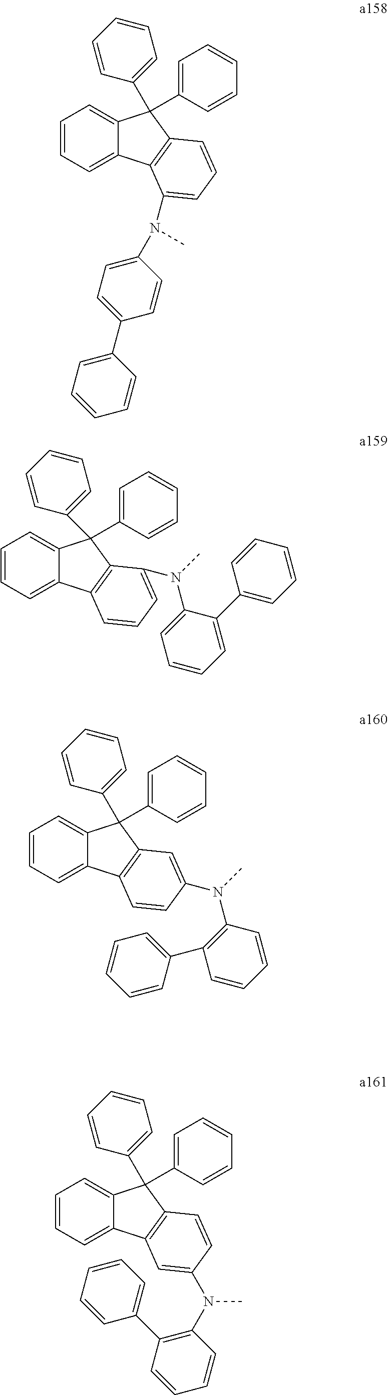

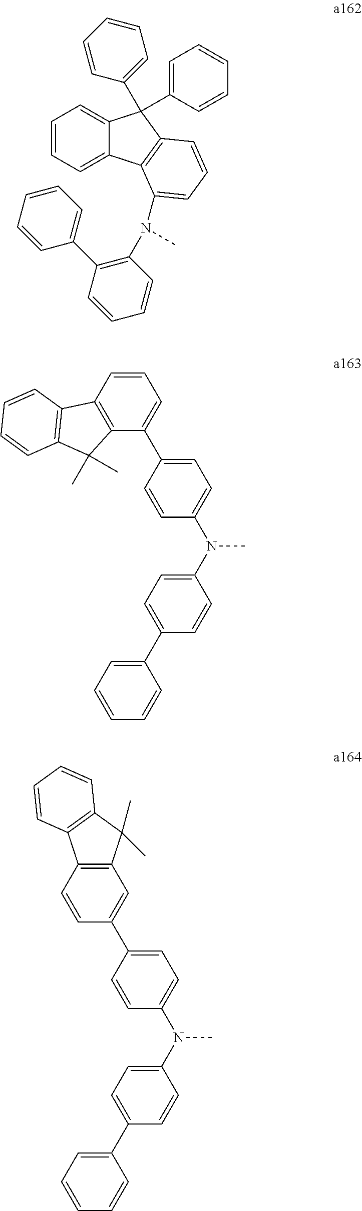

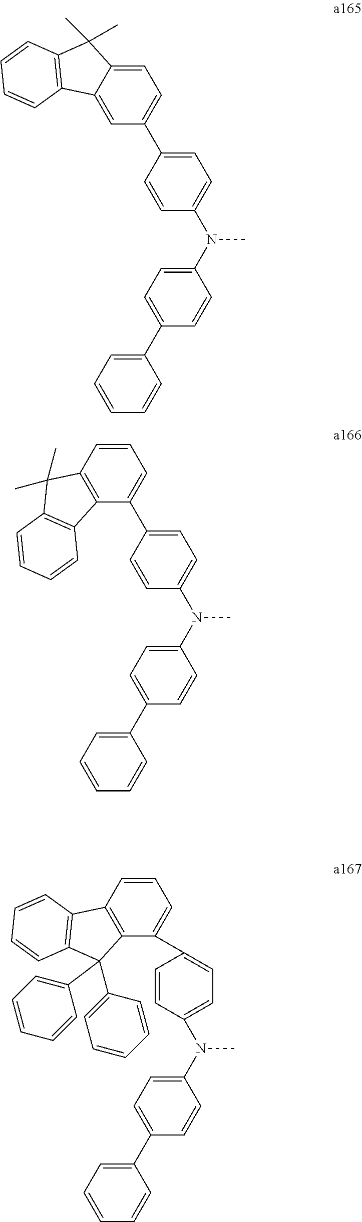

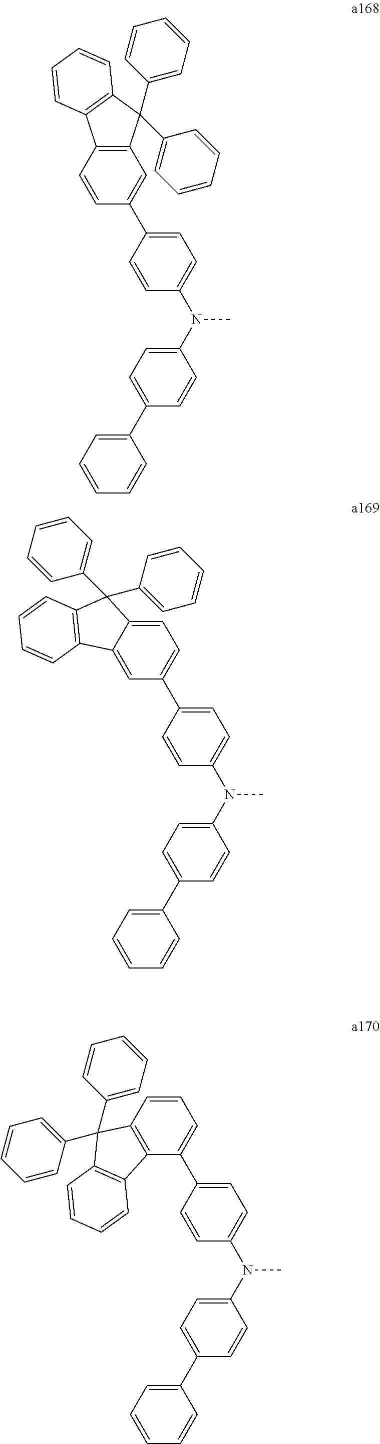

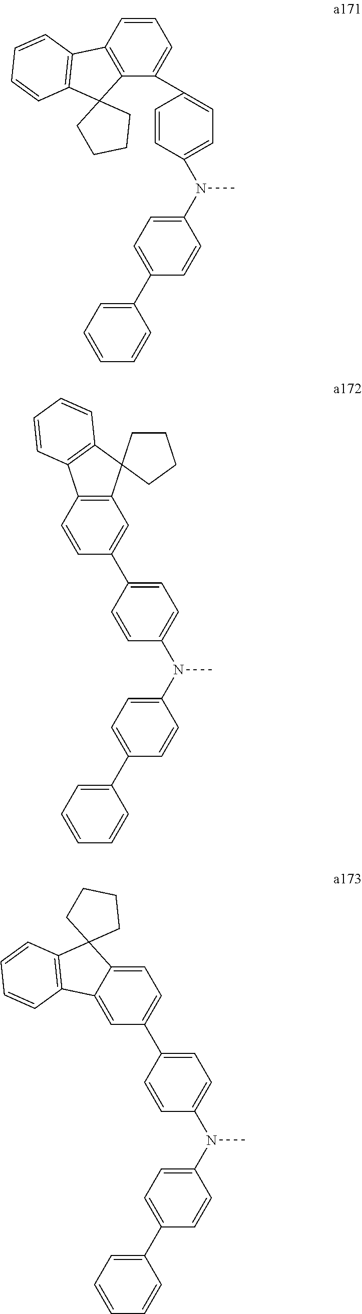

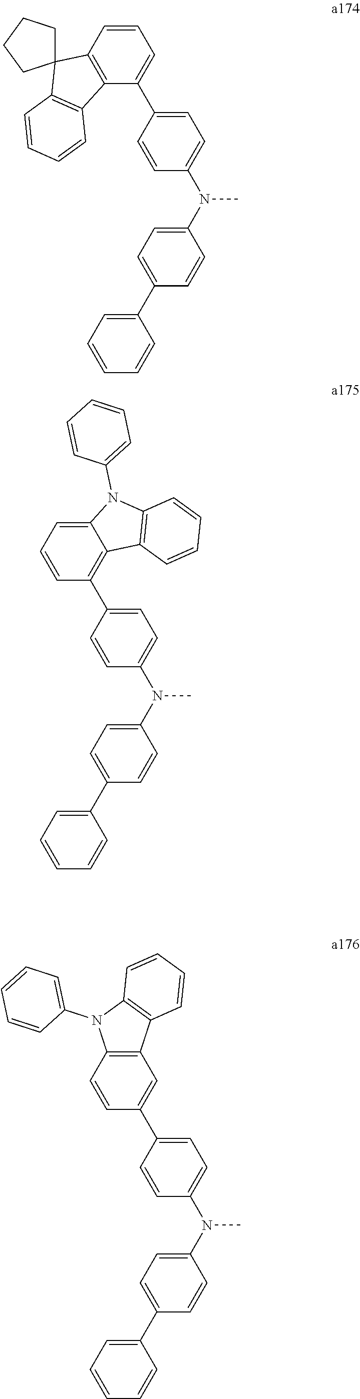

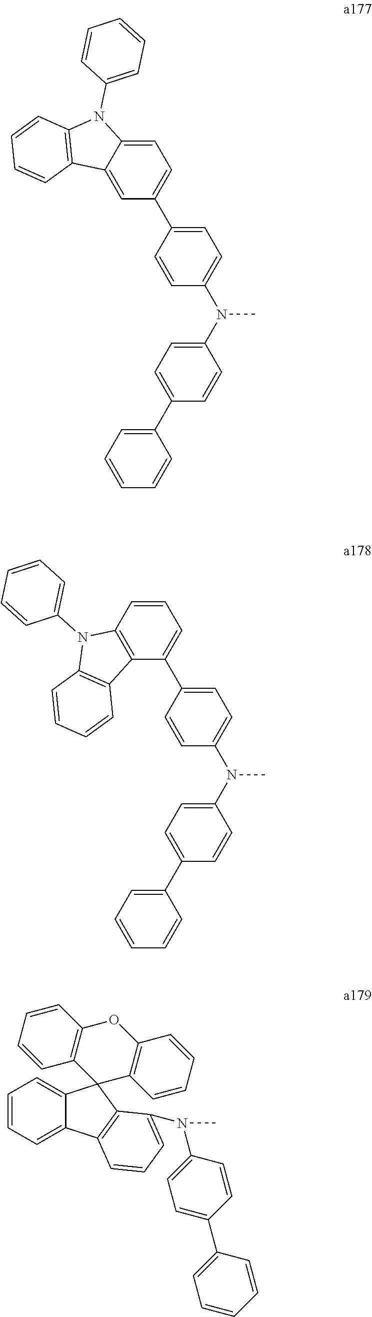

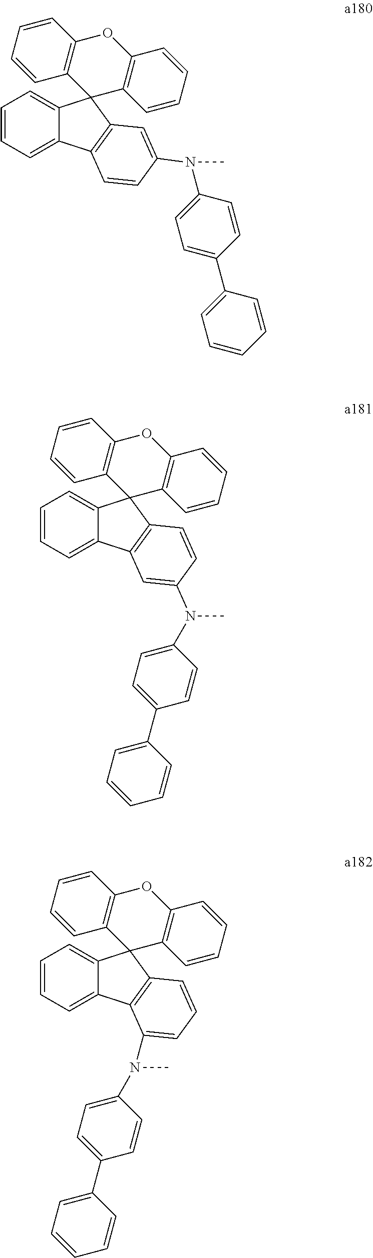

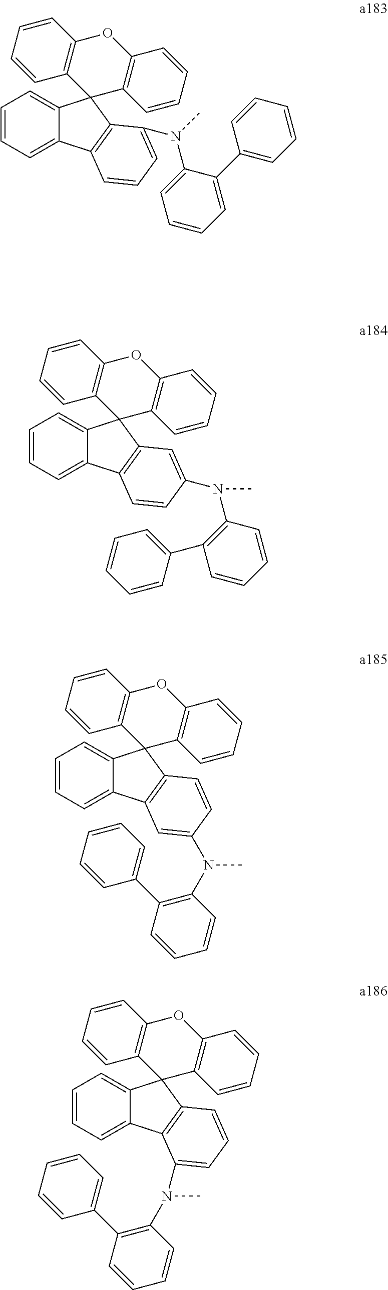

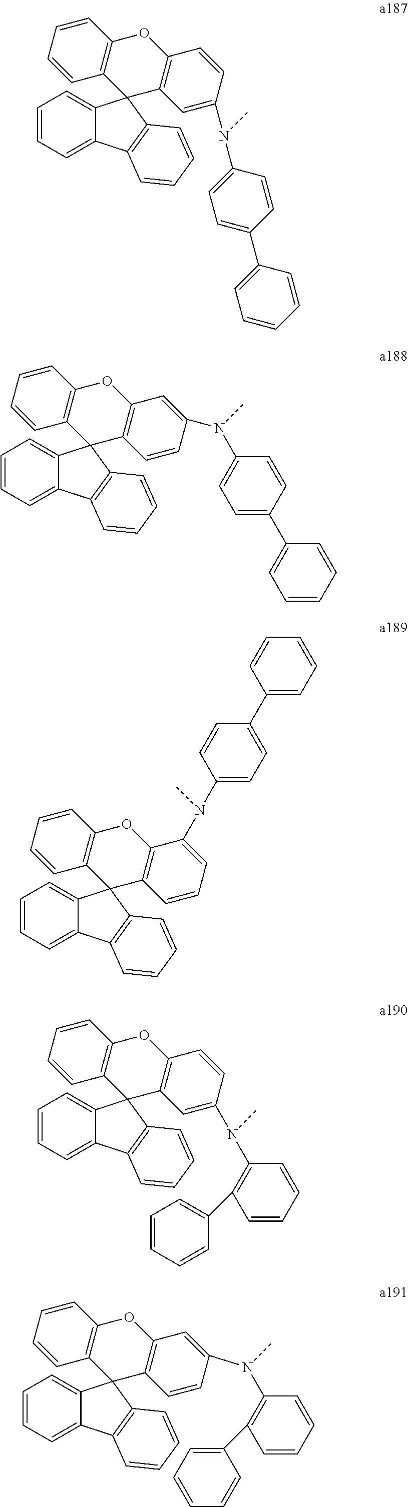

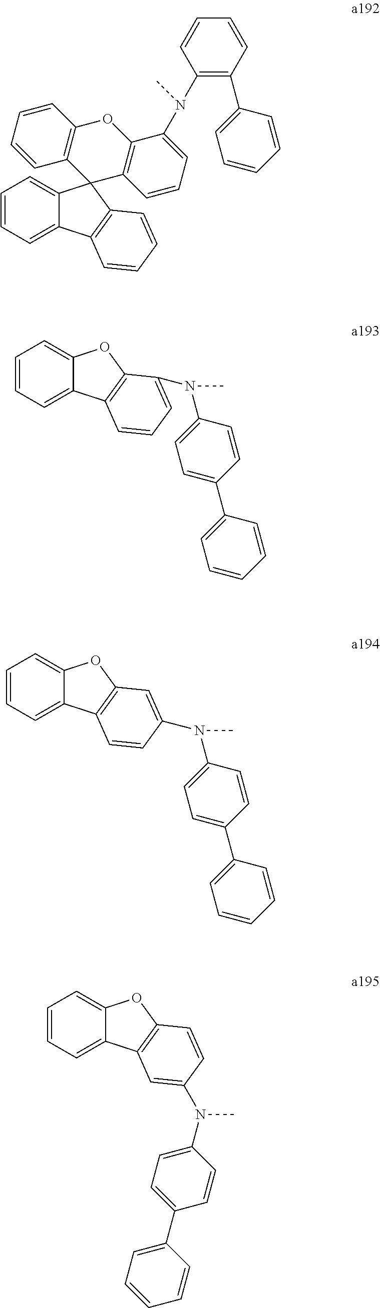

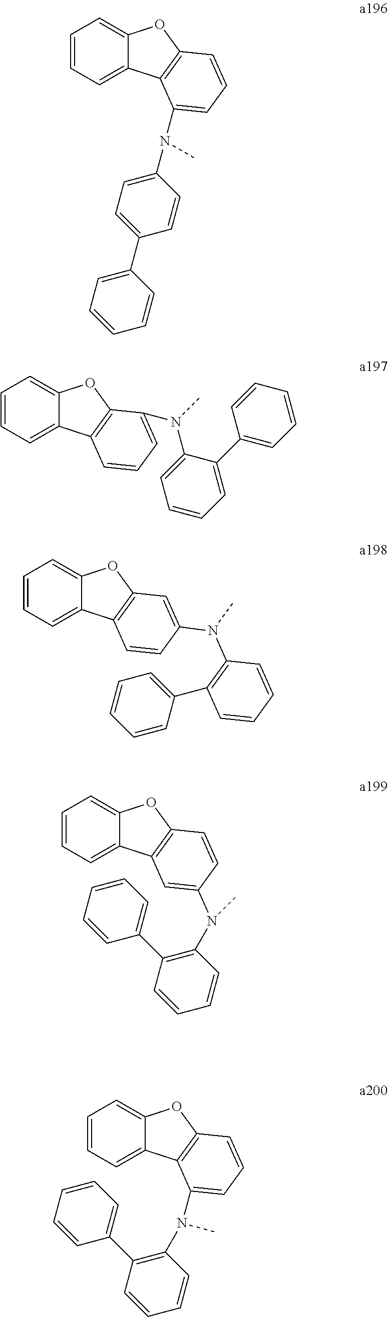

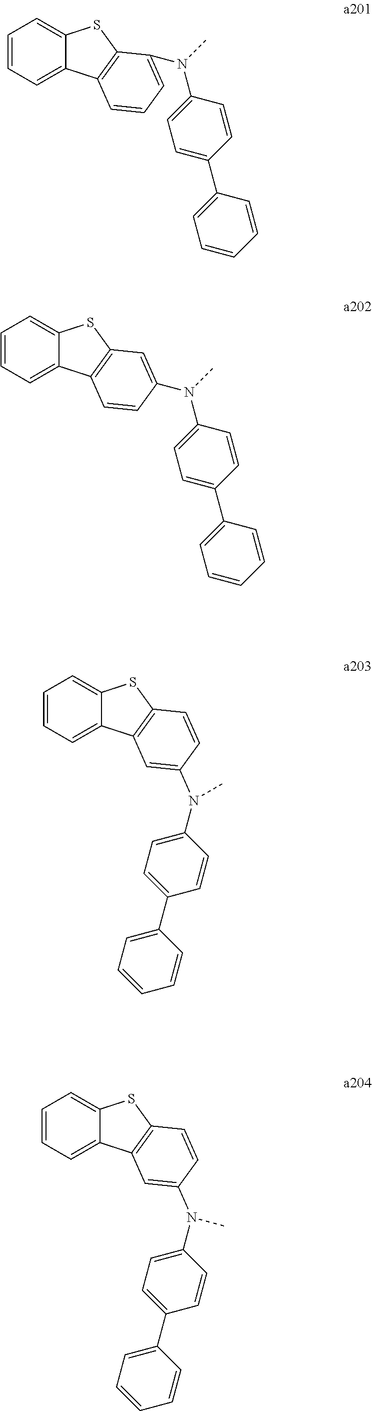

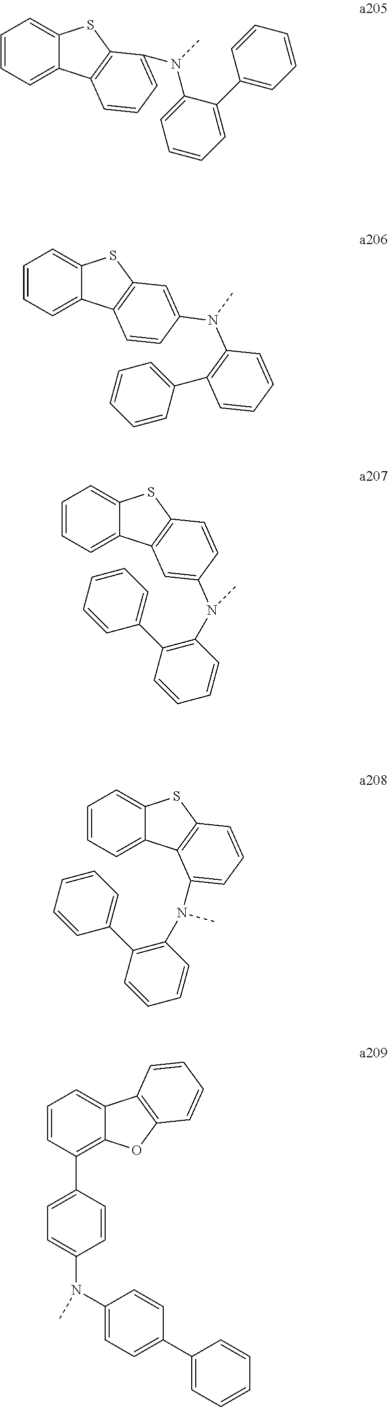

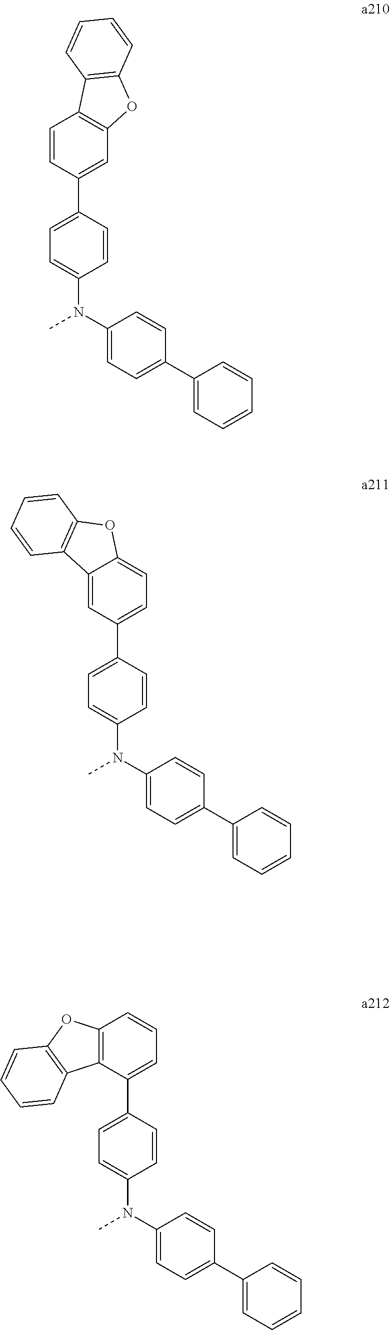

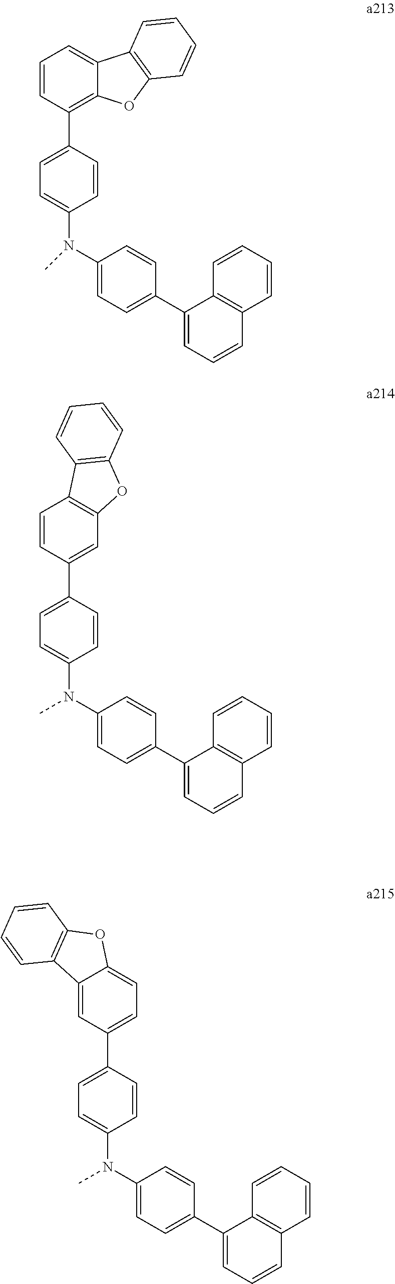

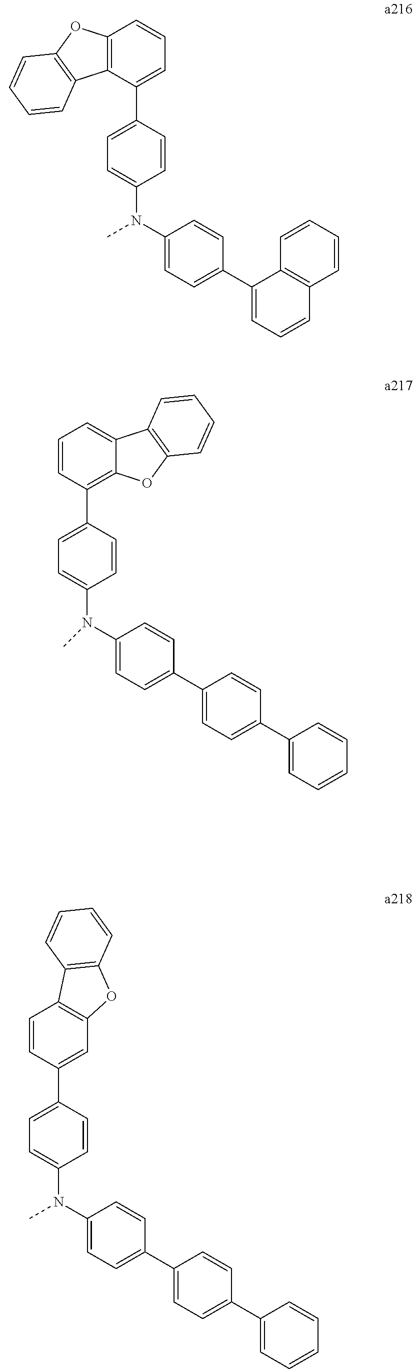

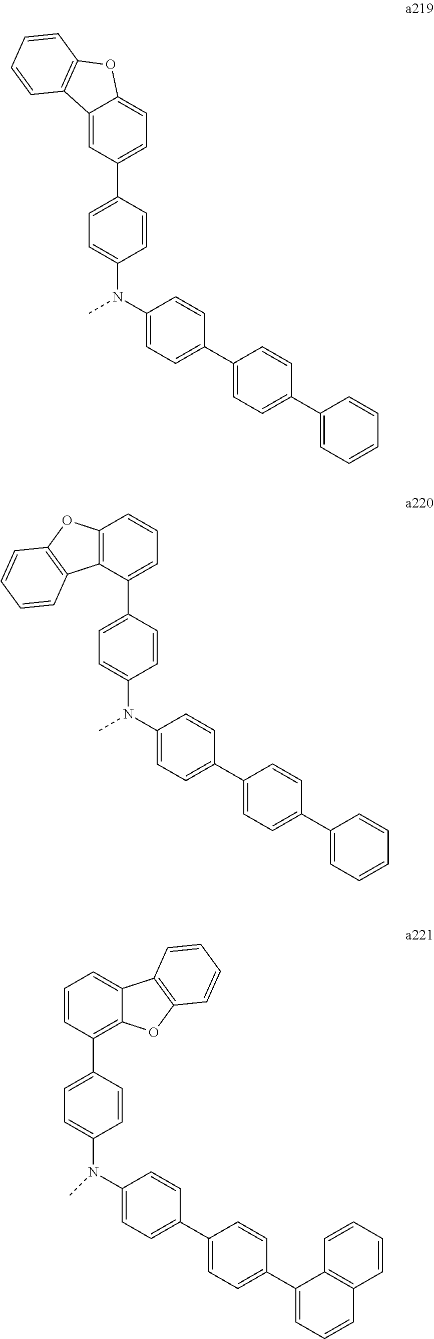

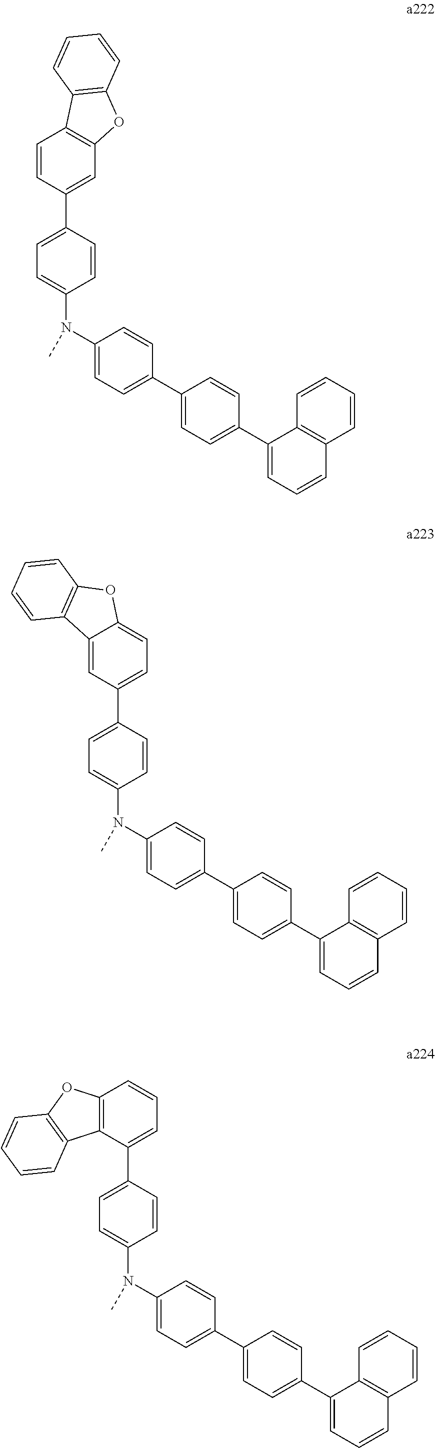

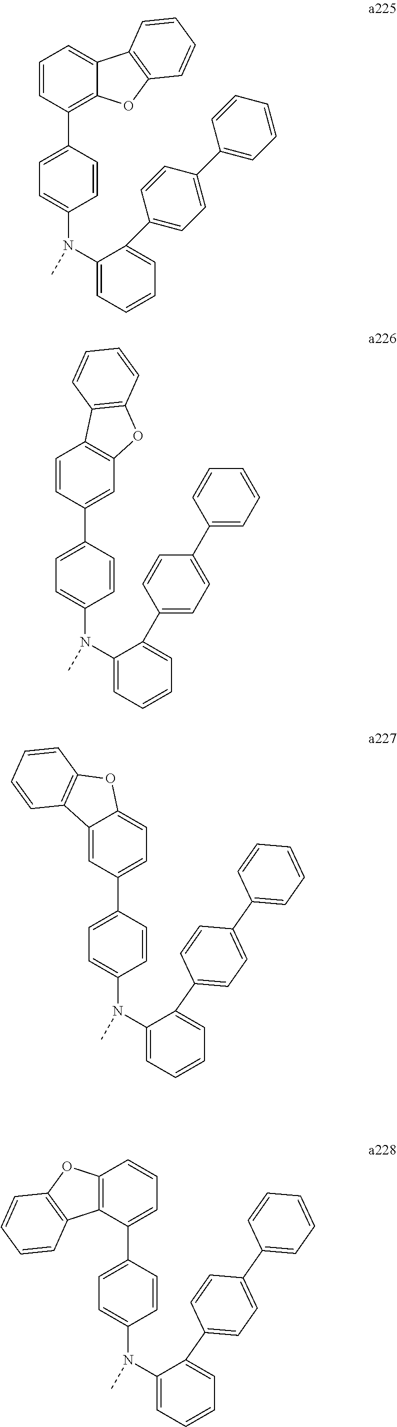

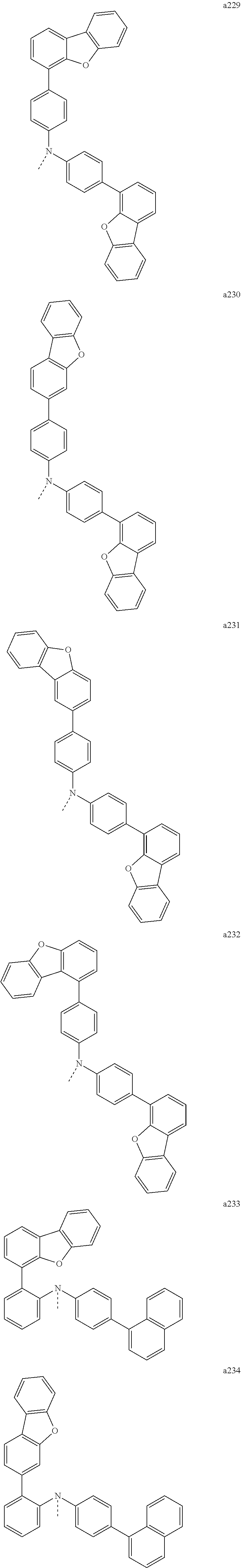

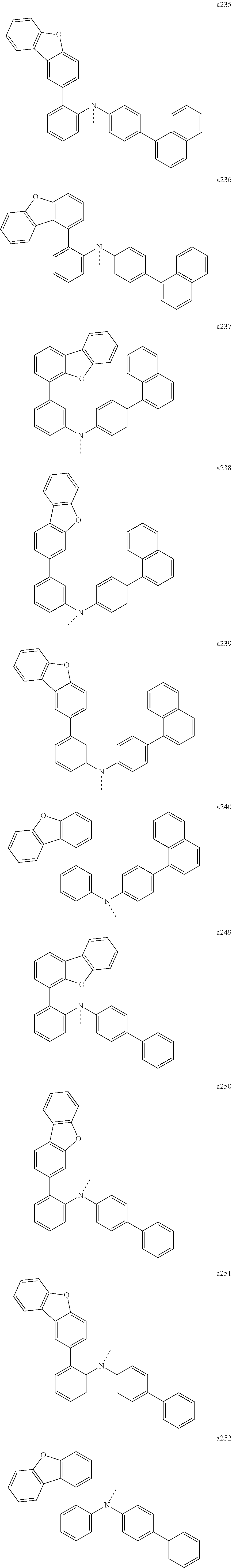

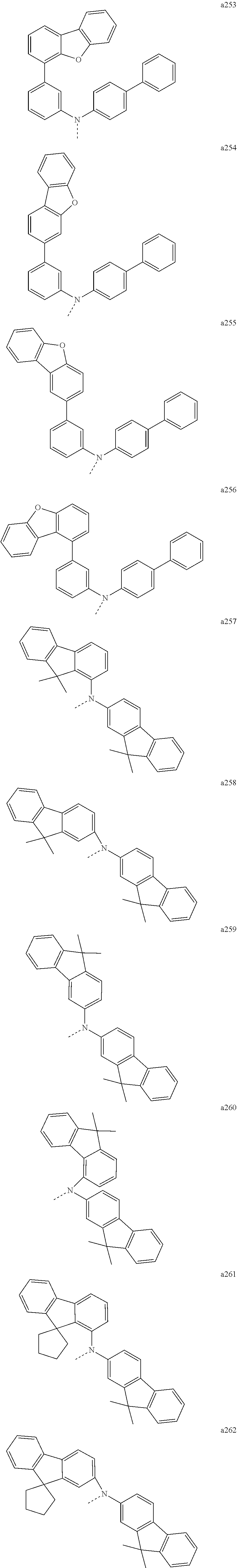

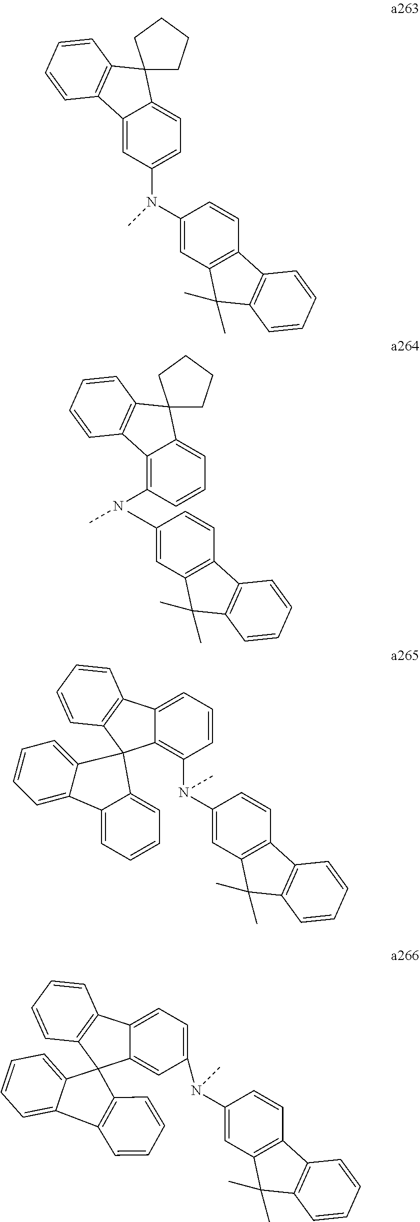

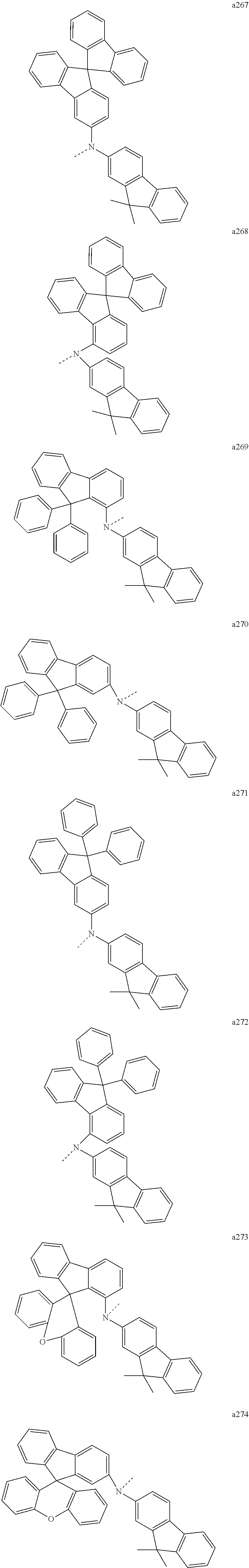

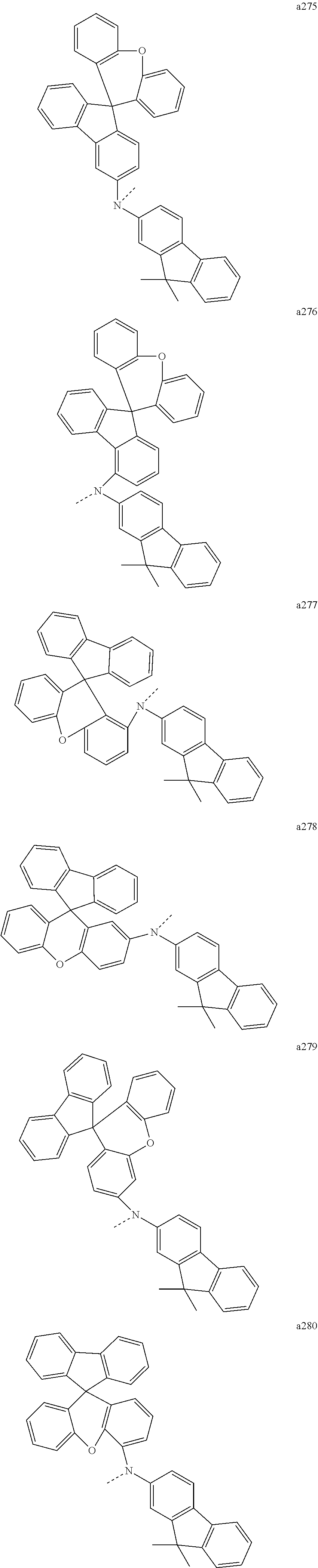

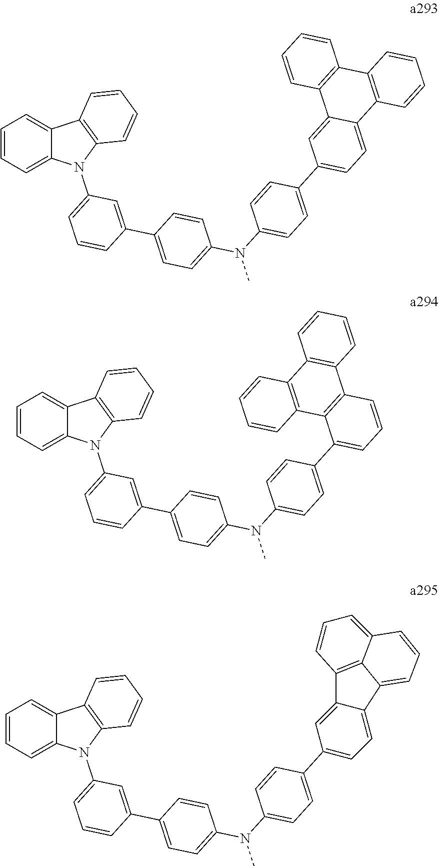

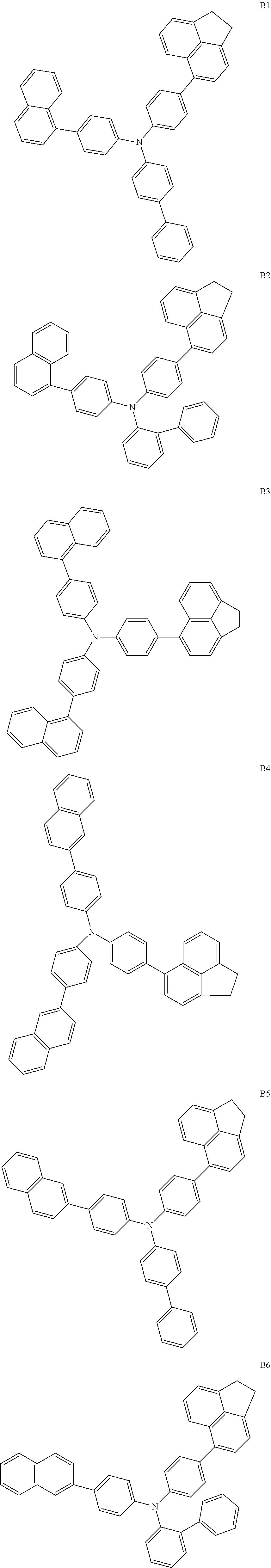

[symbol]

represents a chemical bond.

The substituted group W3 is formed by replacing one or more hydrogens on the unsubstituted group W3 with deuterium. For example, the substituted group W3 may be formed by replacing all hydrogens on the unsubstituted group W3 with deuterium.

In an embodiment of the second type of aromatic amine compounds, the aromatic amine compound is selected from substituted or unsubstituted compound W4. The unsubstituted compound W4 is selected from the group consisting of the following compounds:

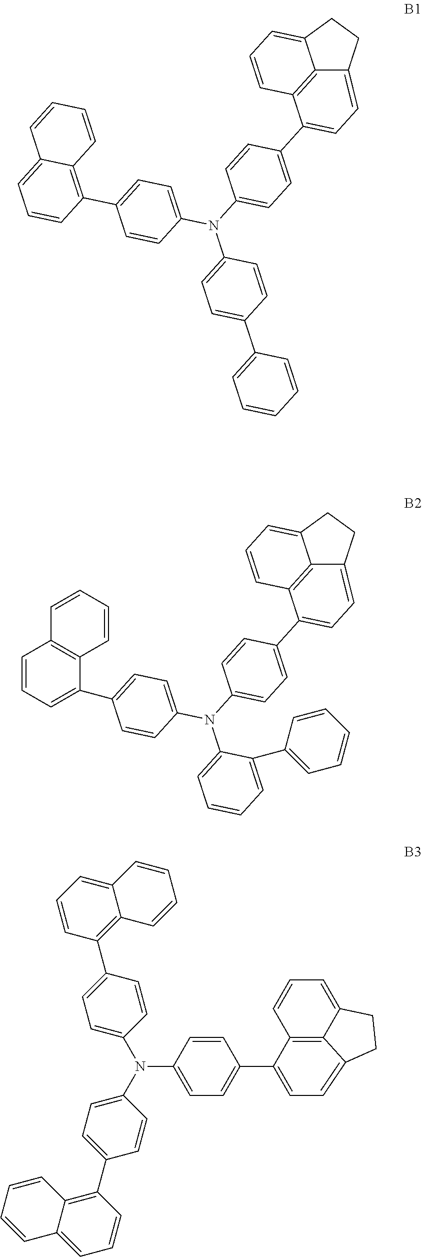

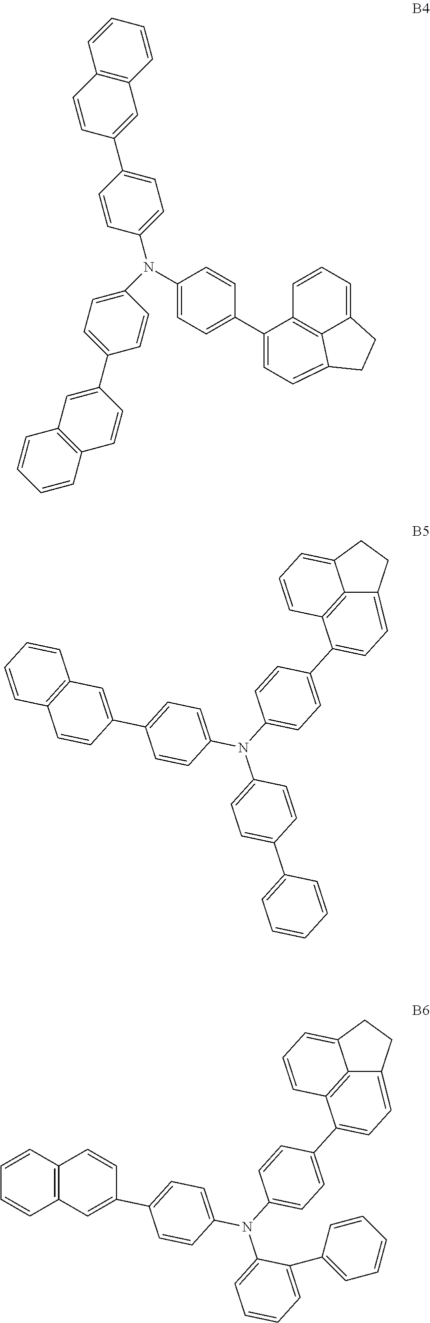

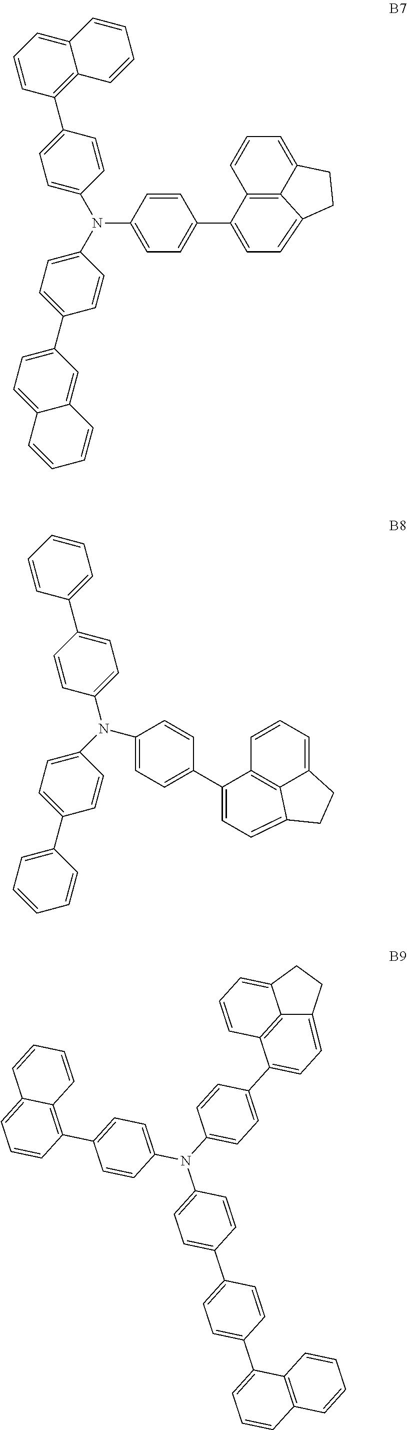

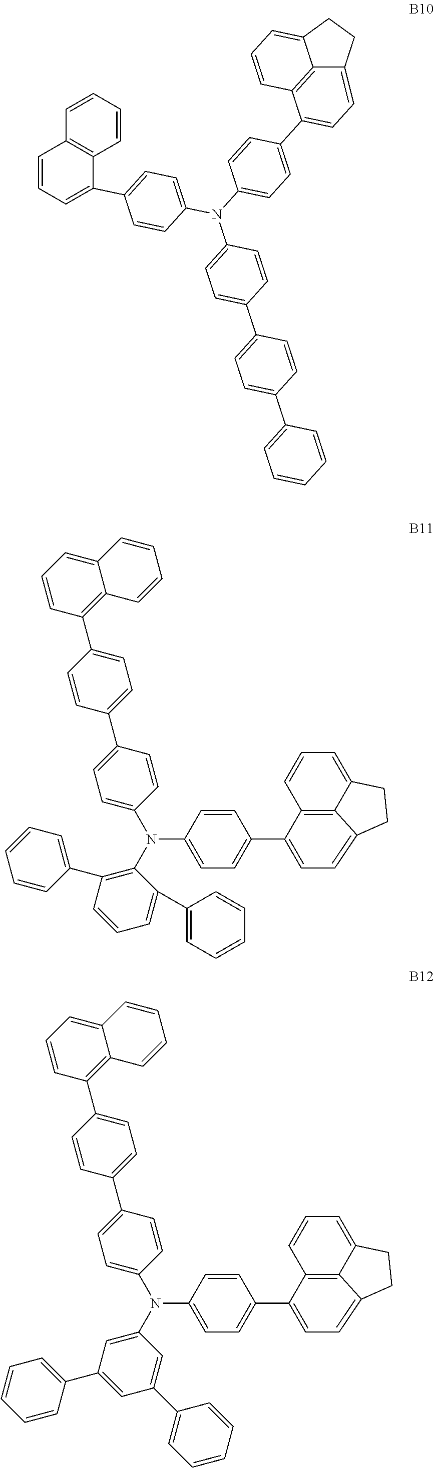

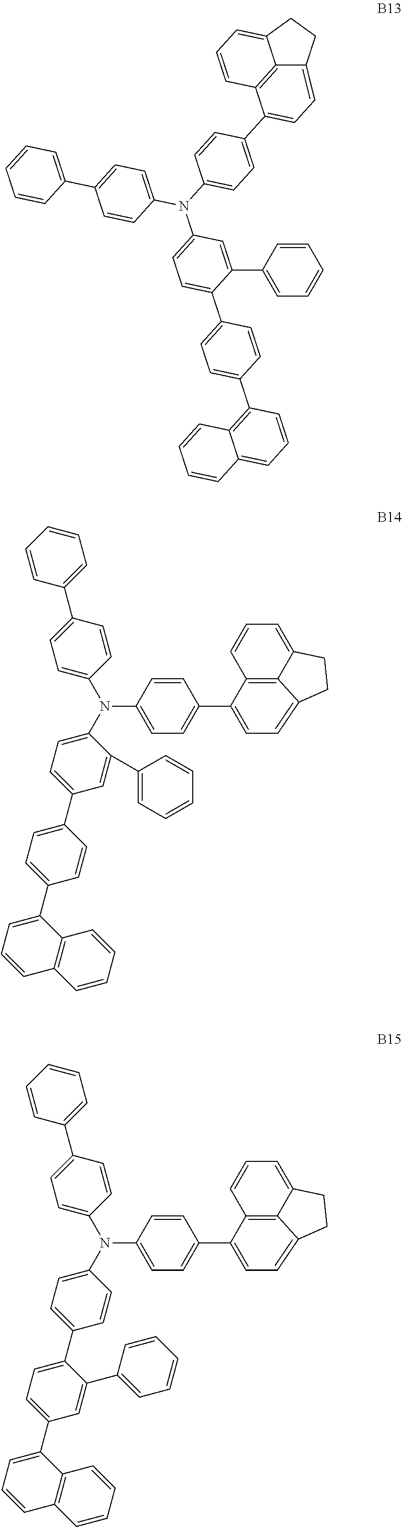

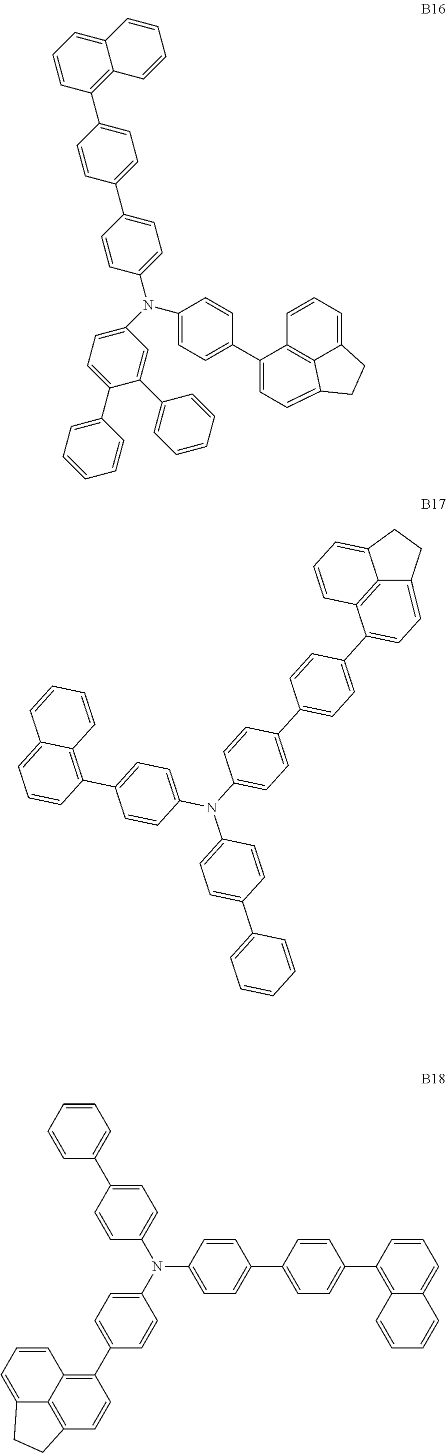

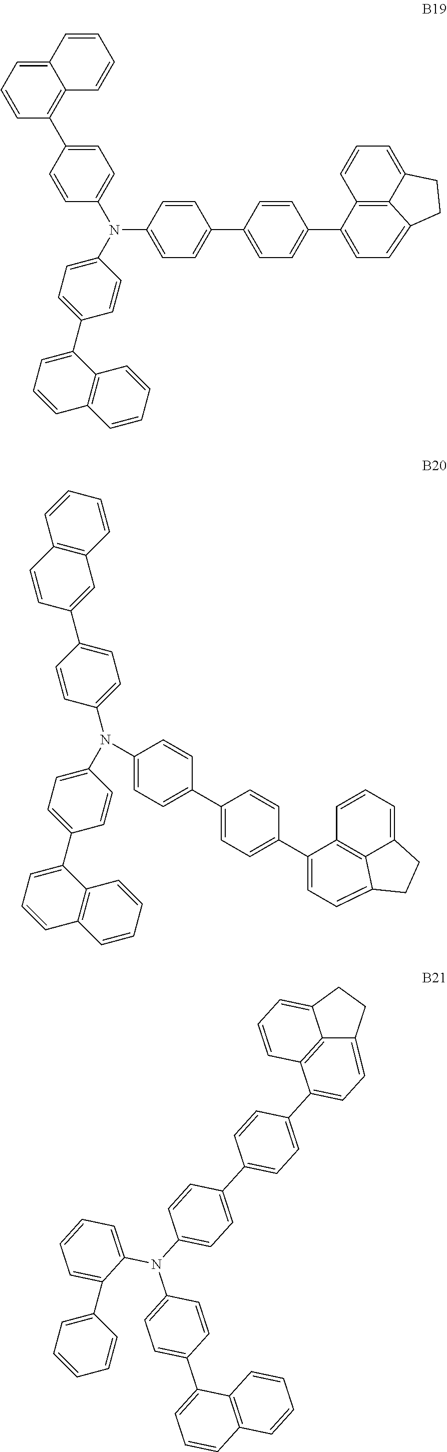

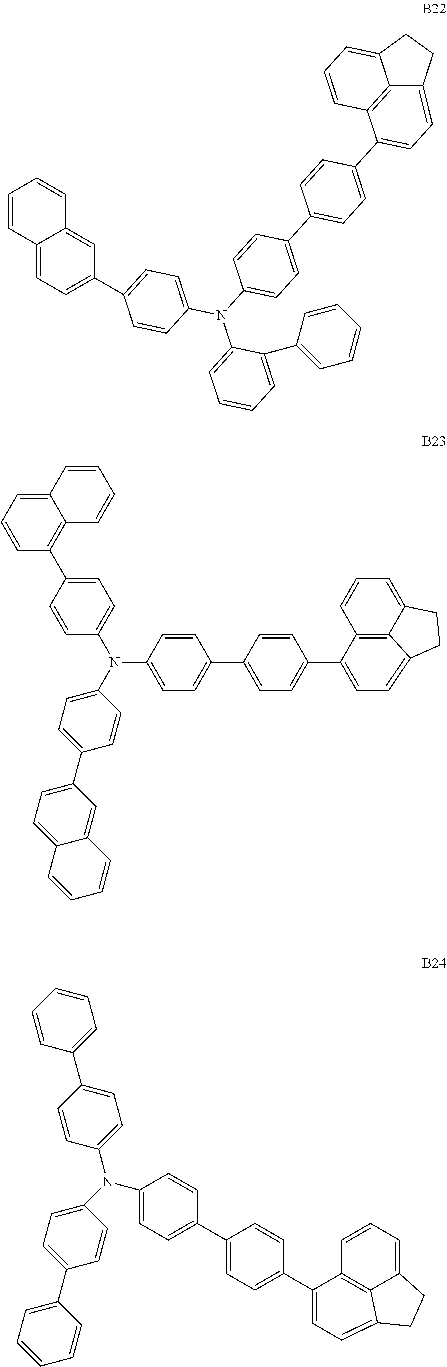

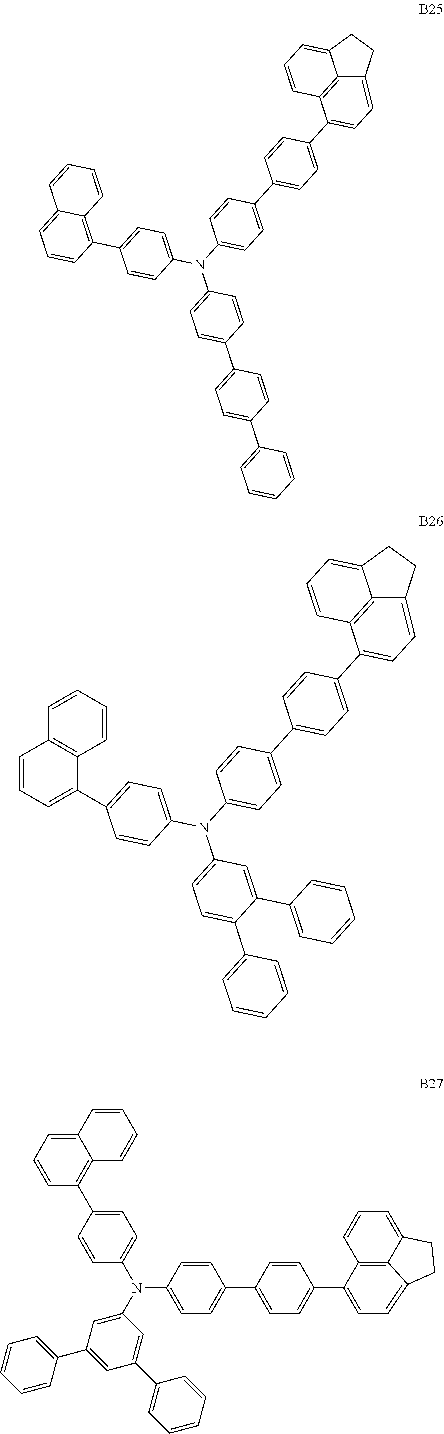

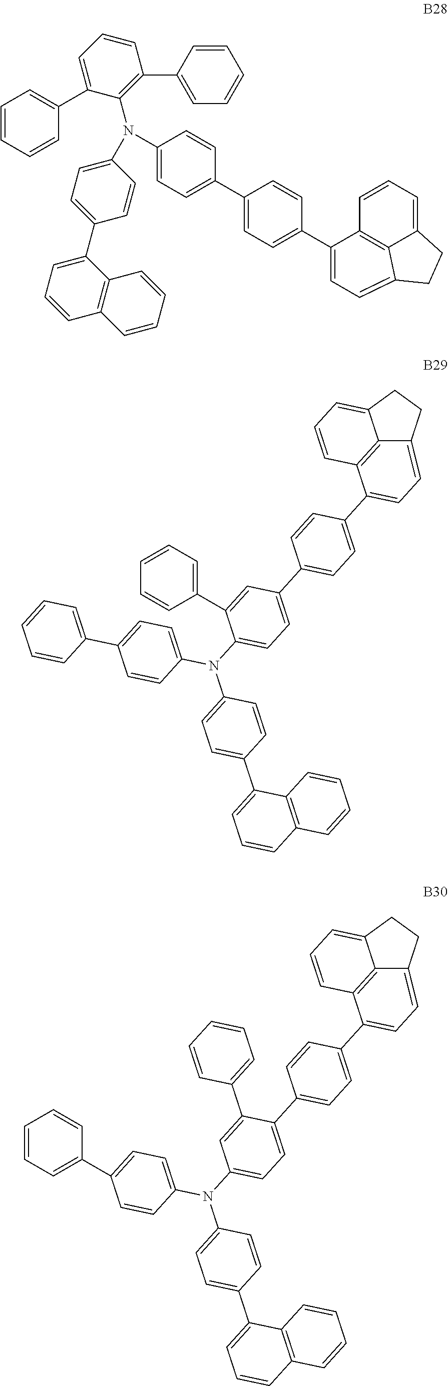

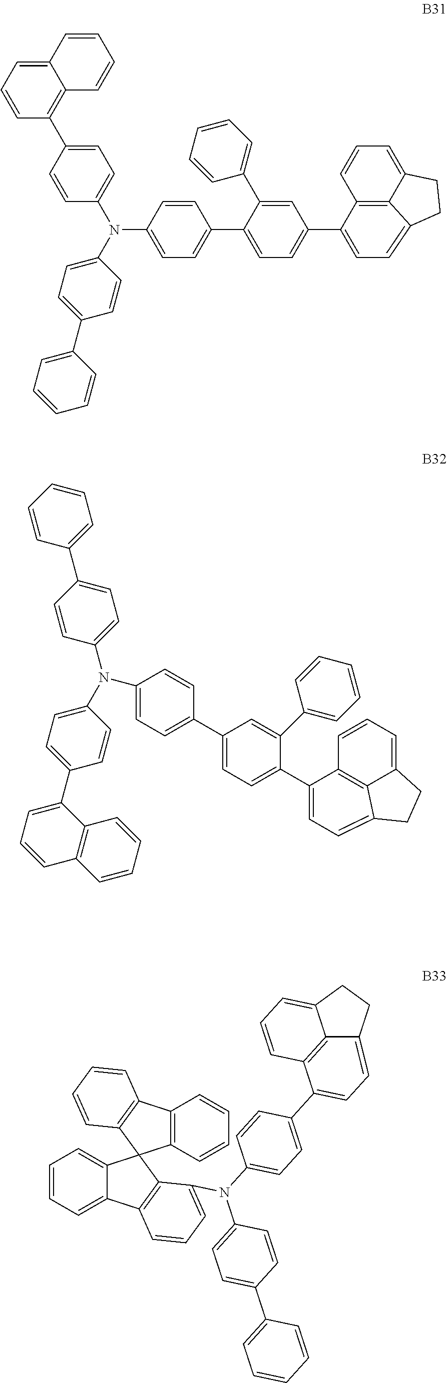

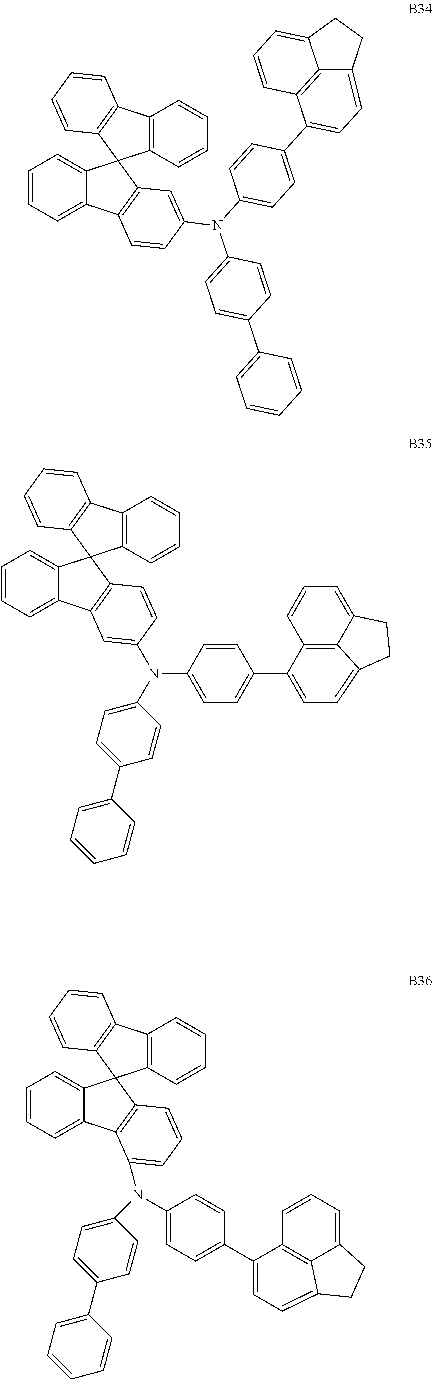

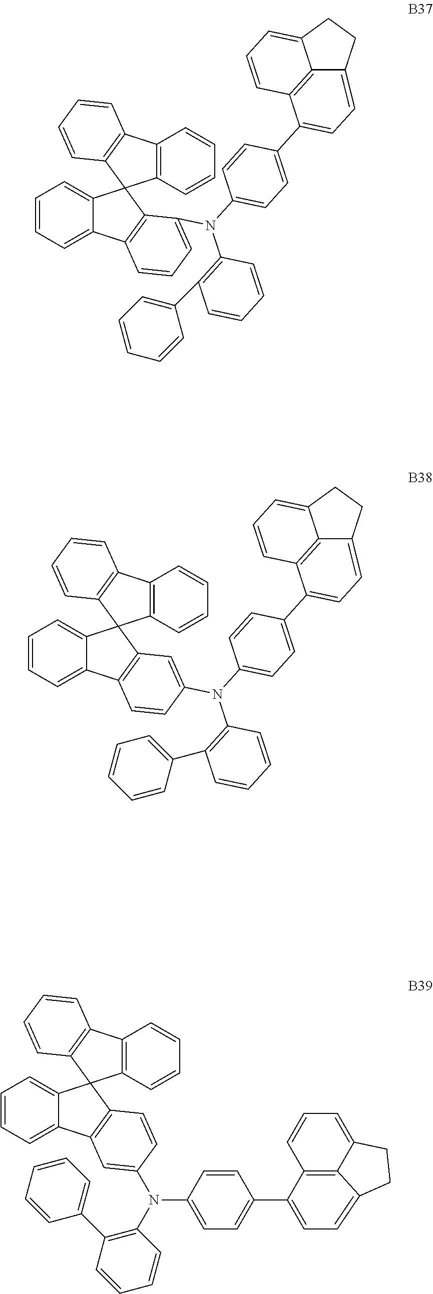

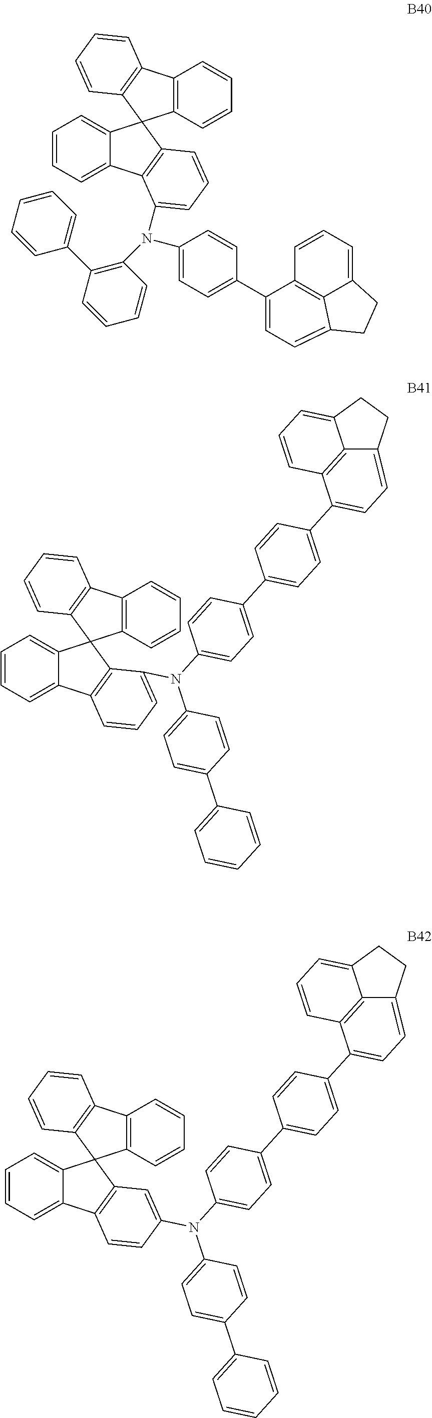

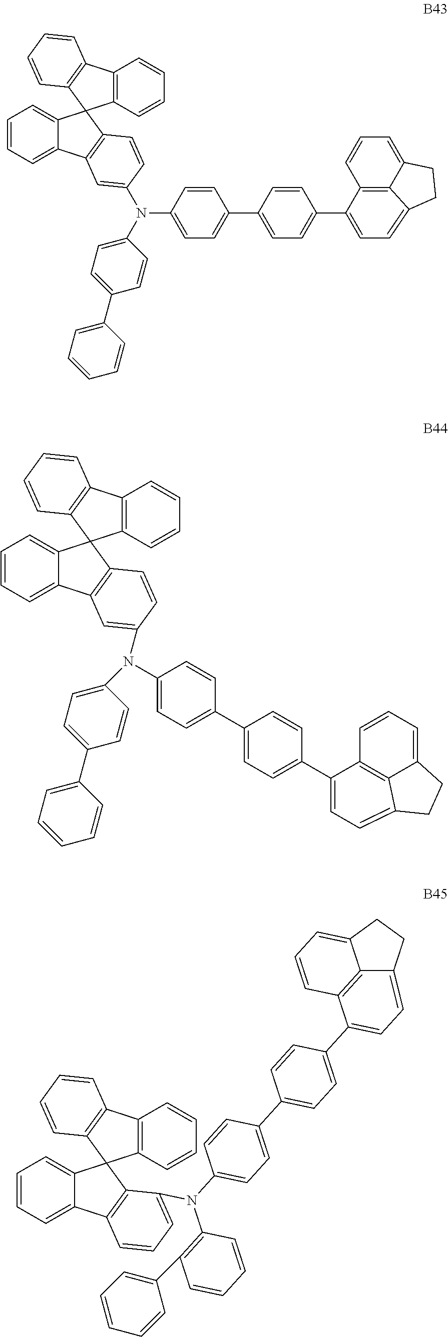

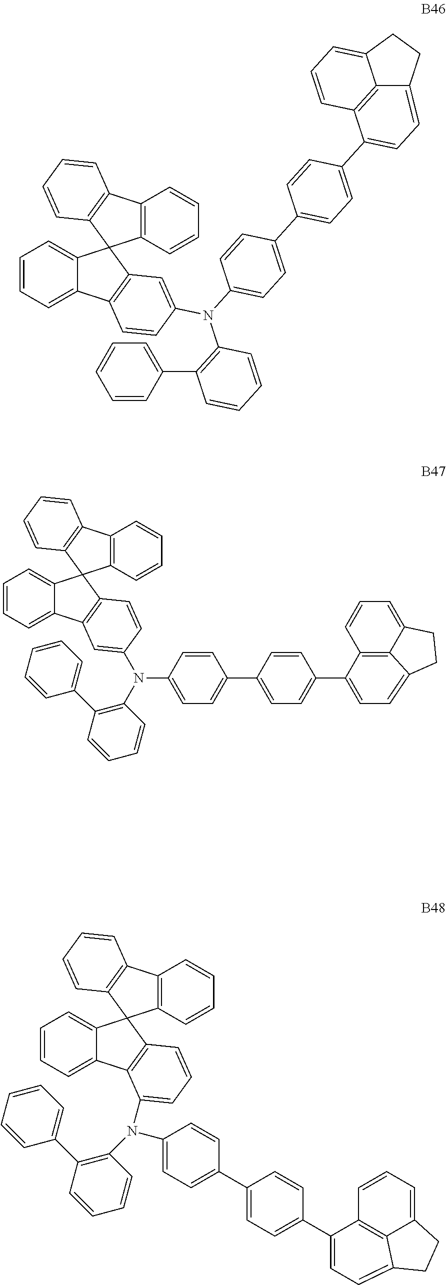

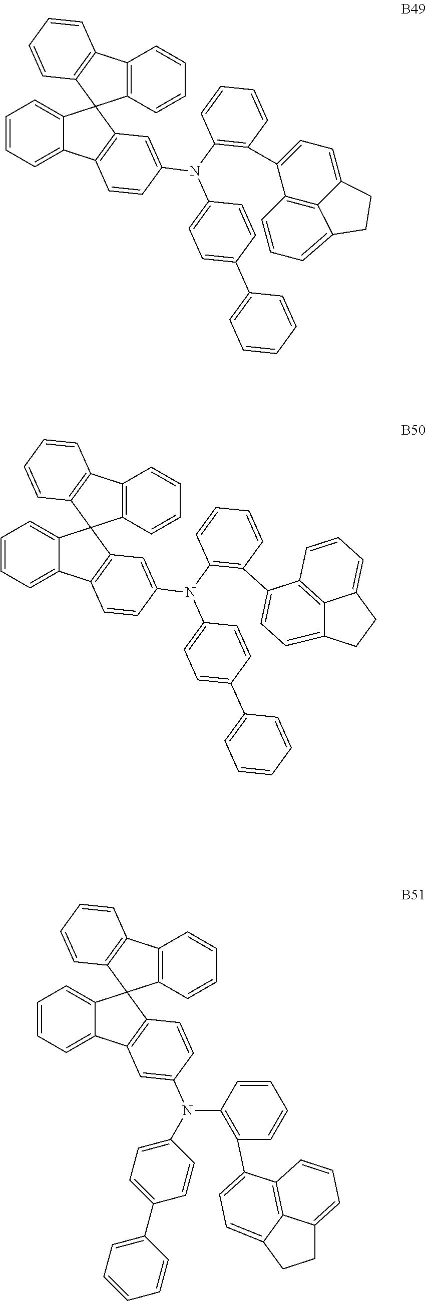

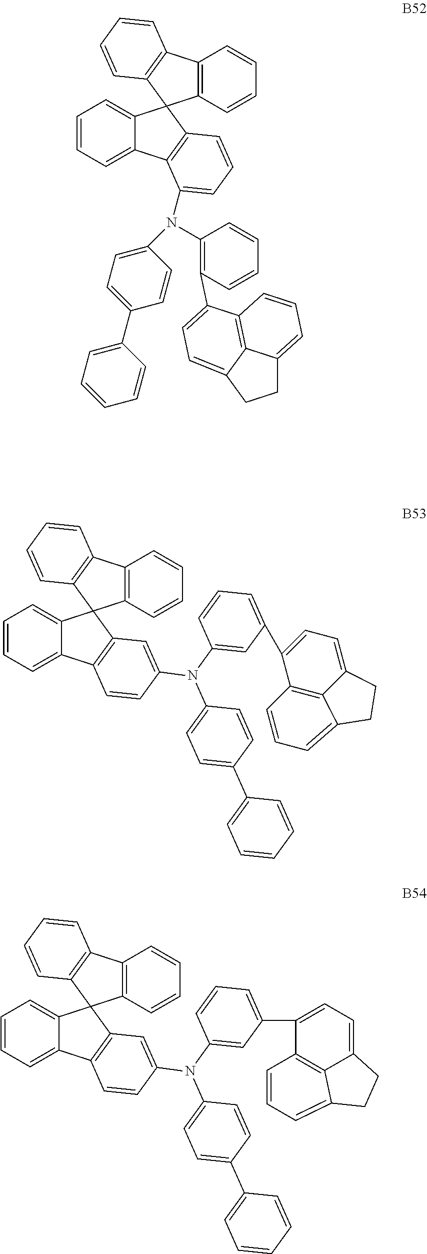

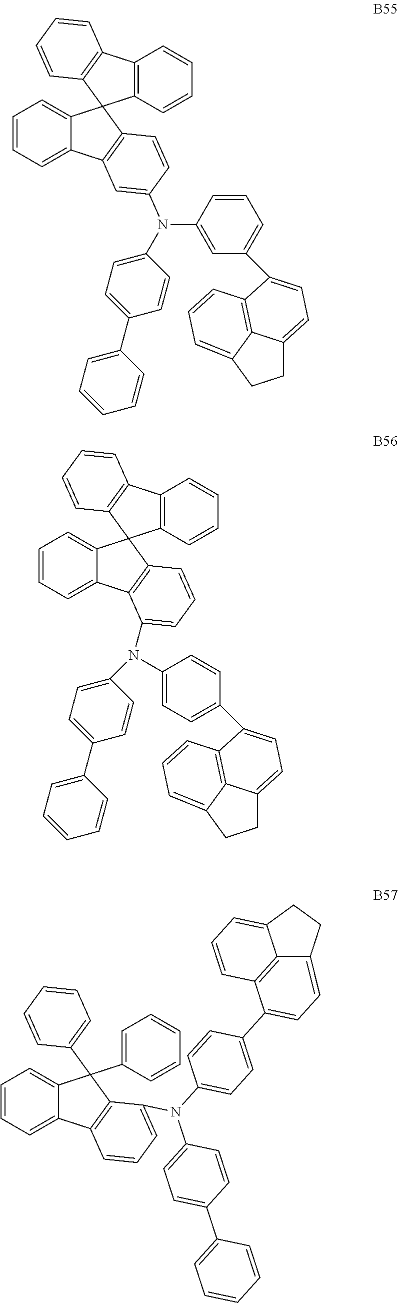

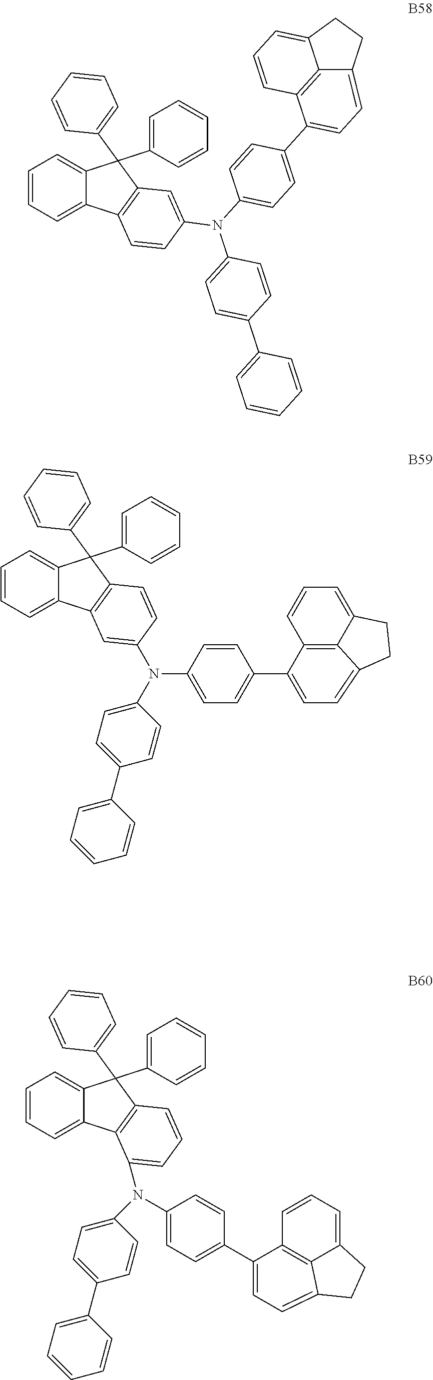

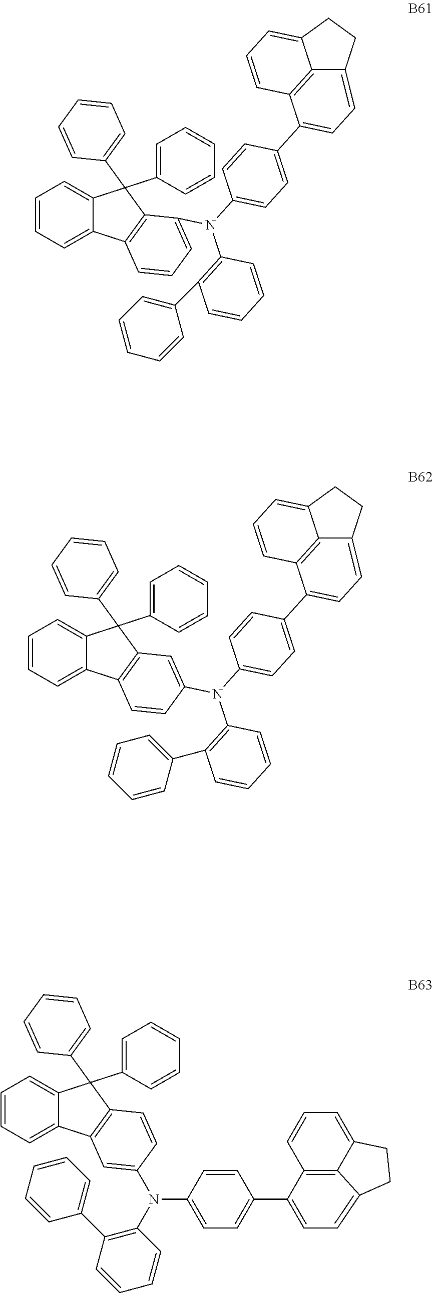

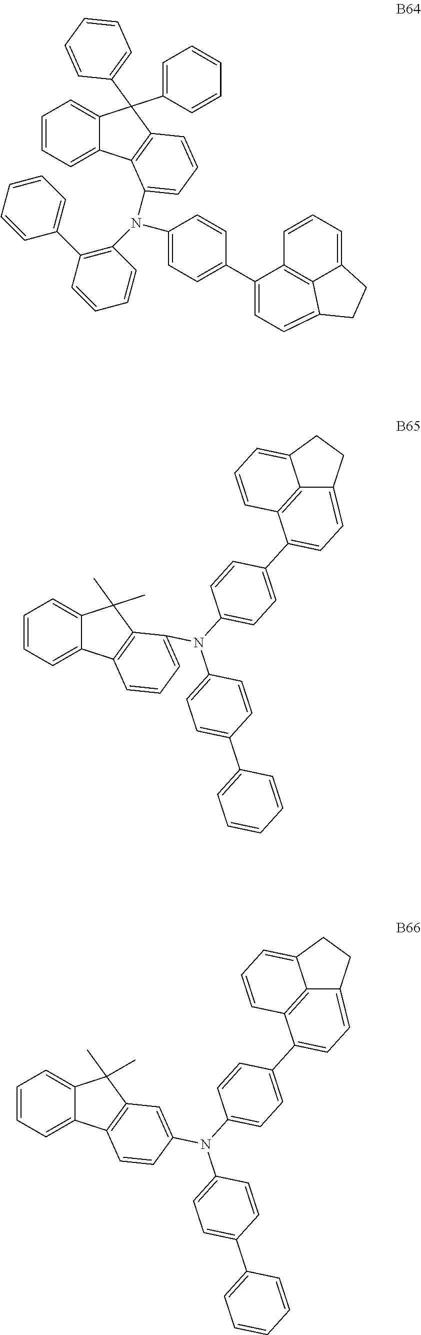

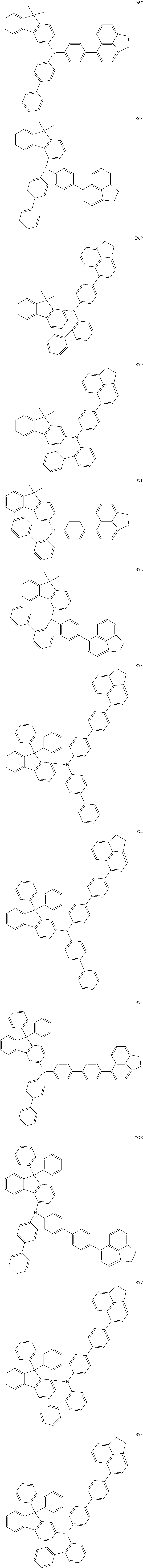

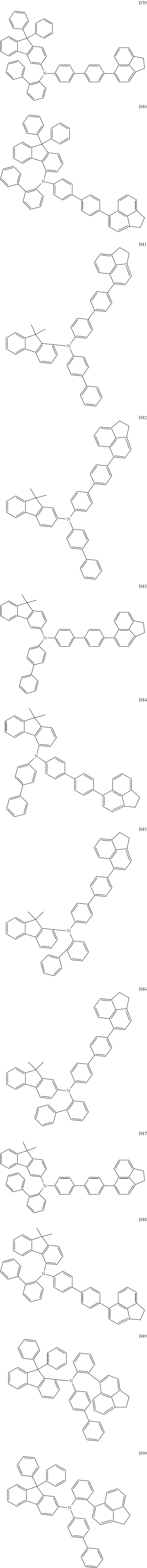

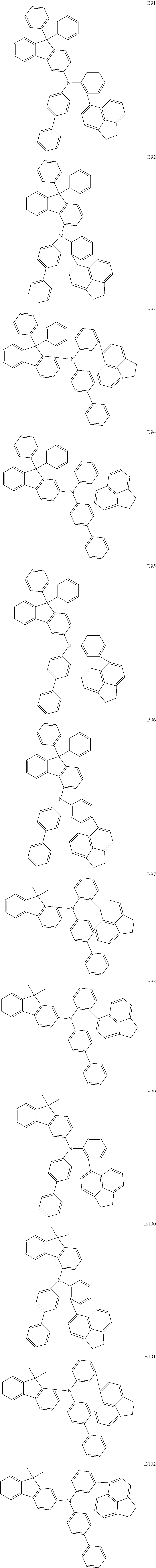

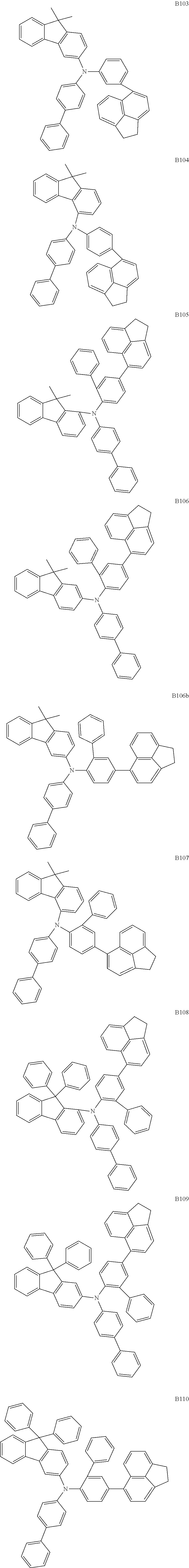

[chemical structure]

[chemical structure]

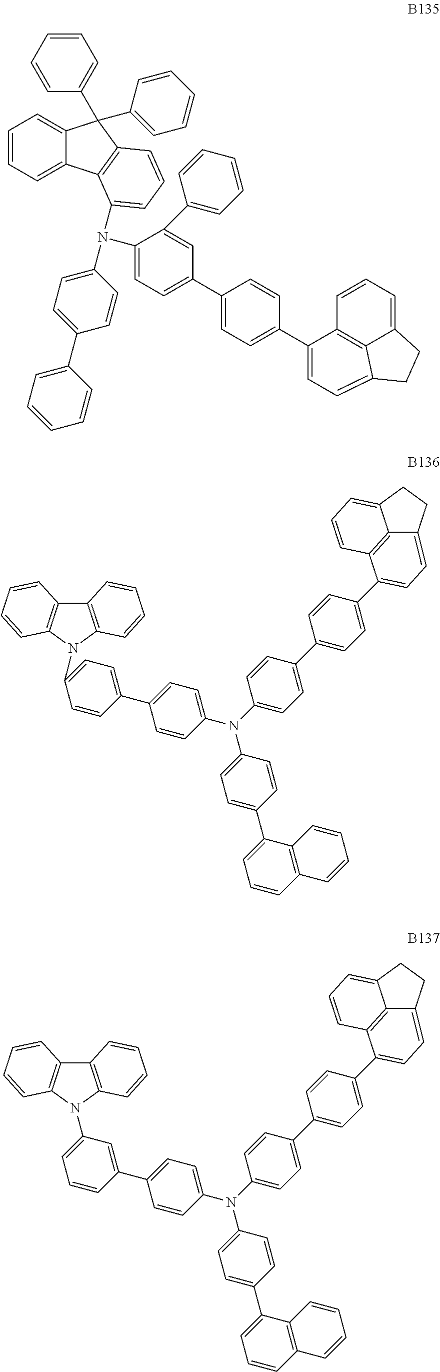

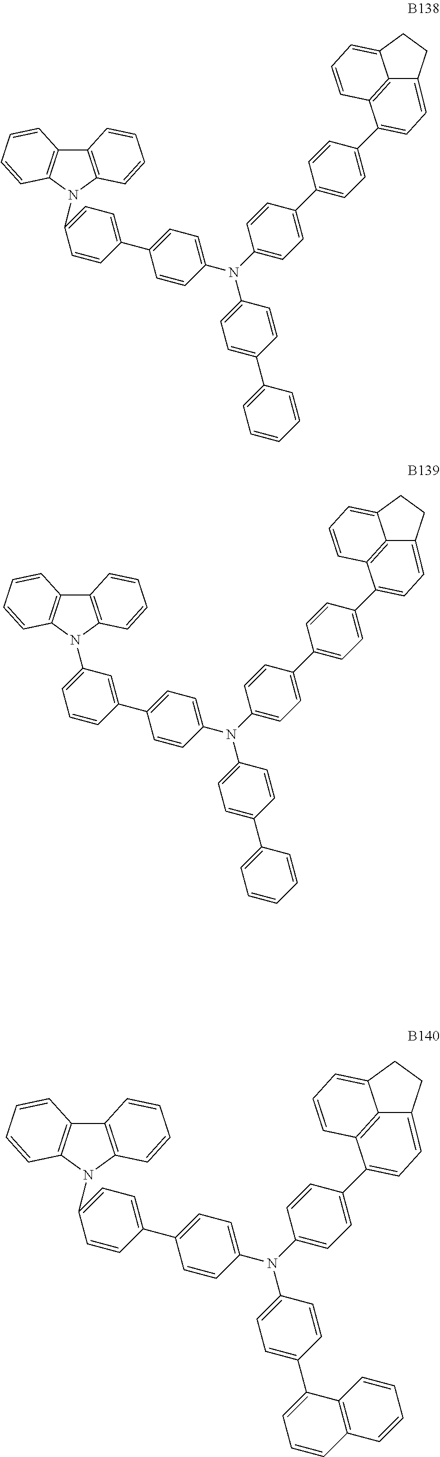

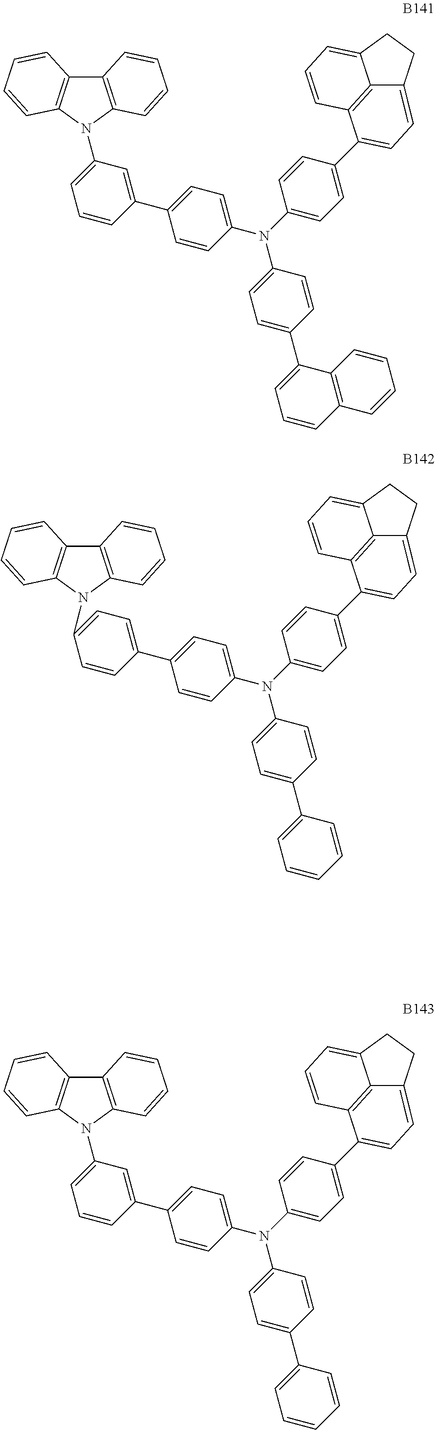

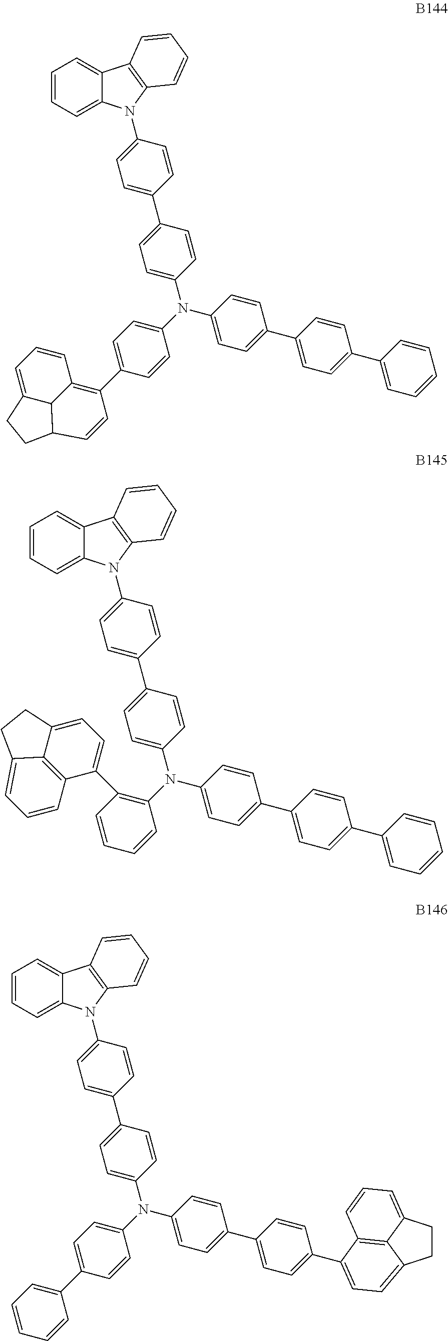

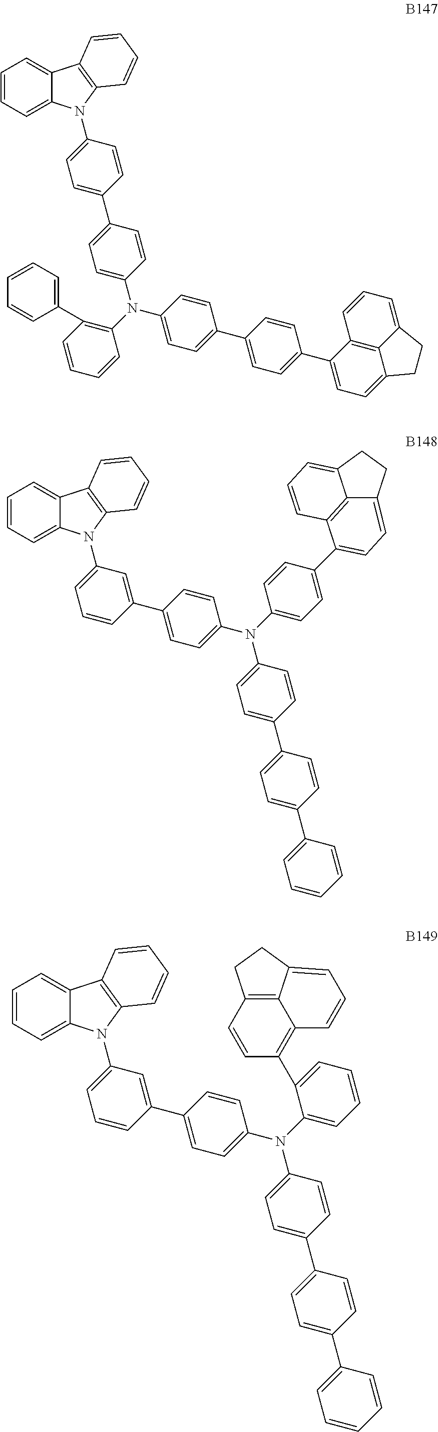

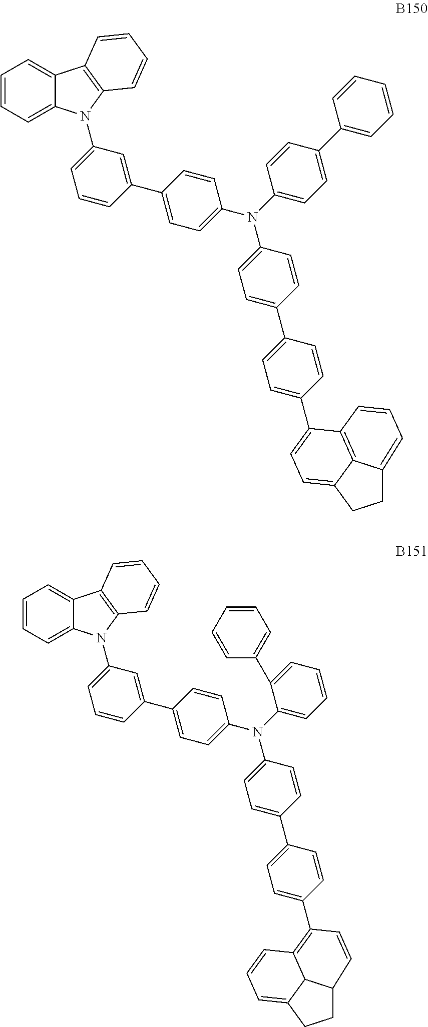

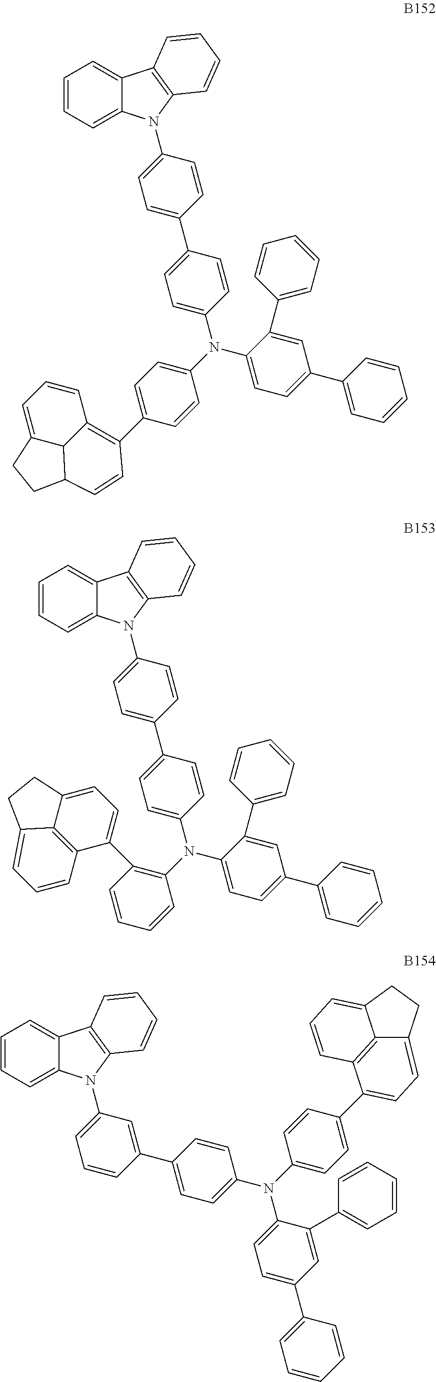

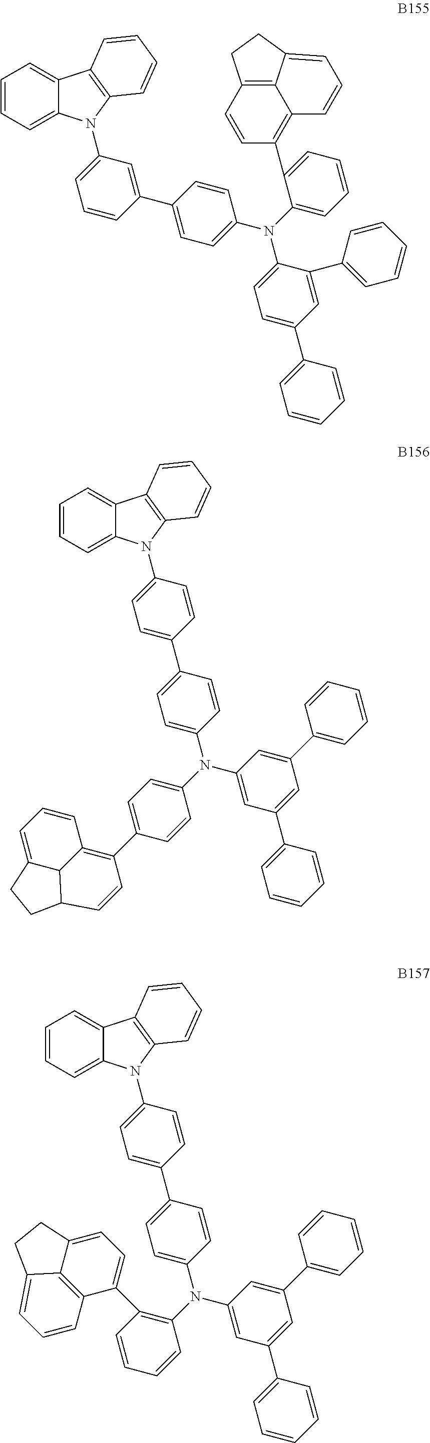

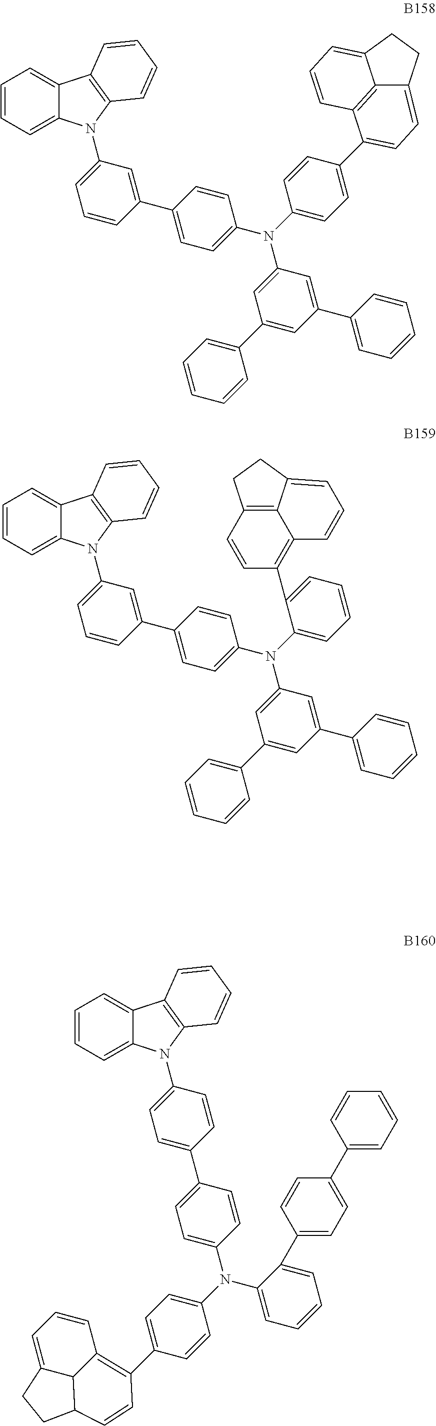

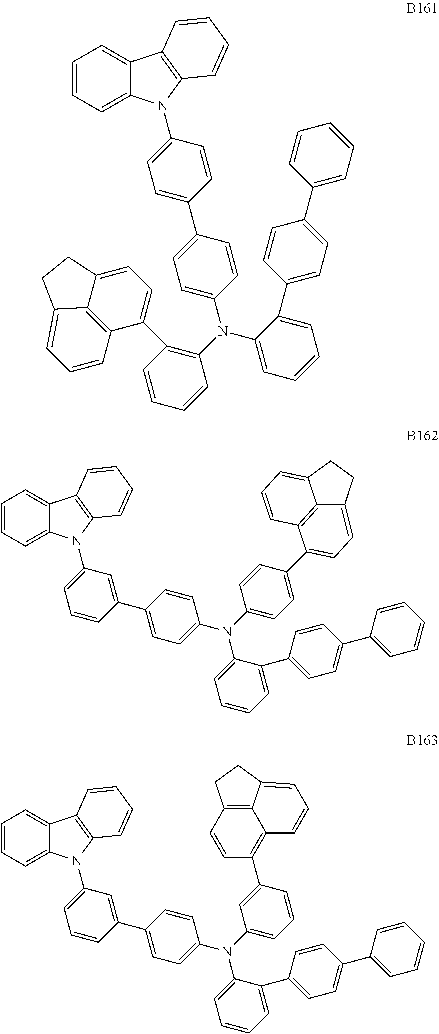

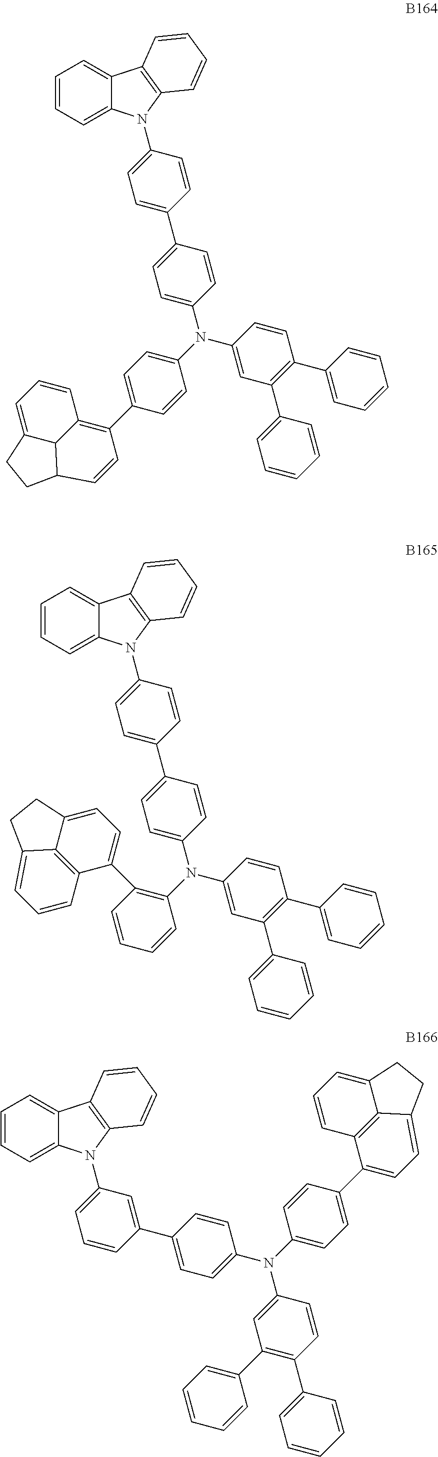

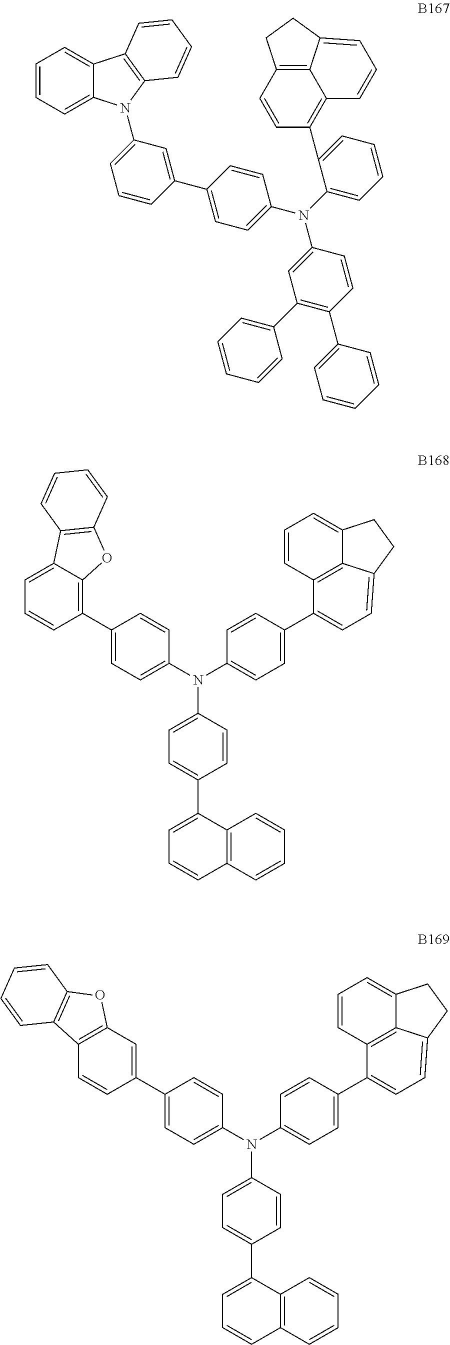

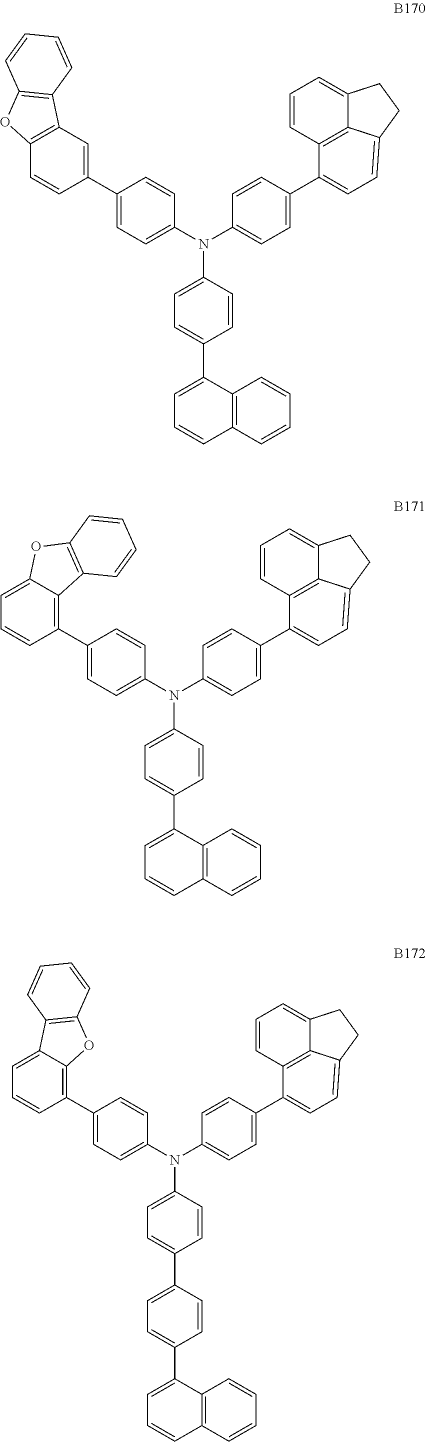

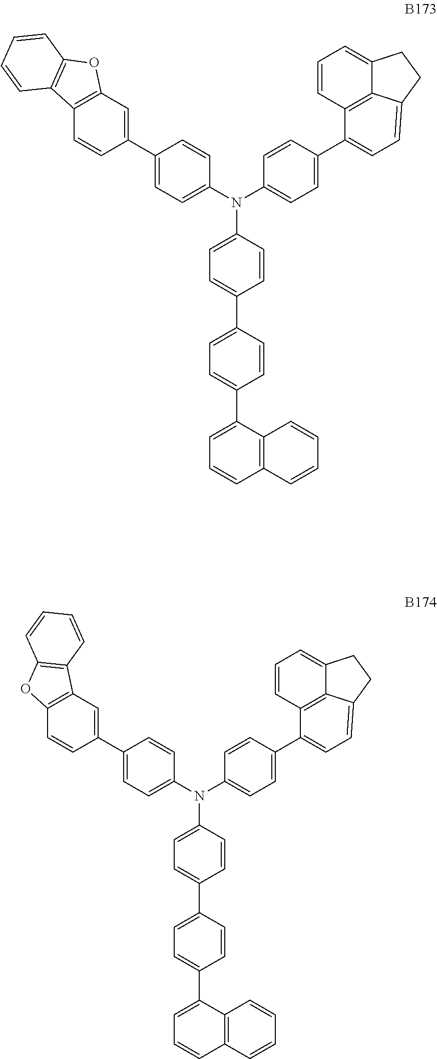

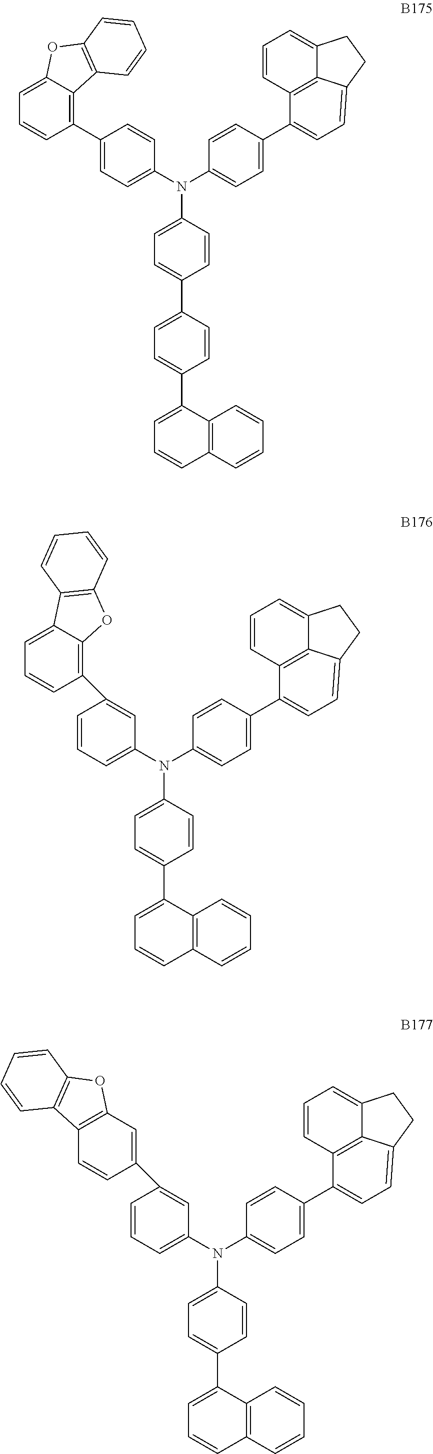

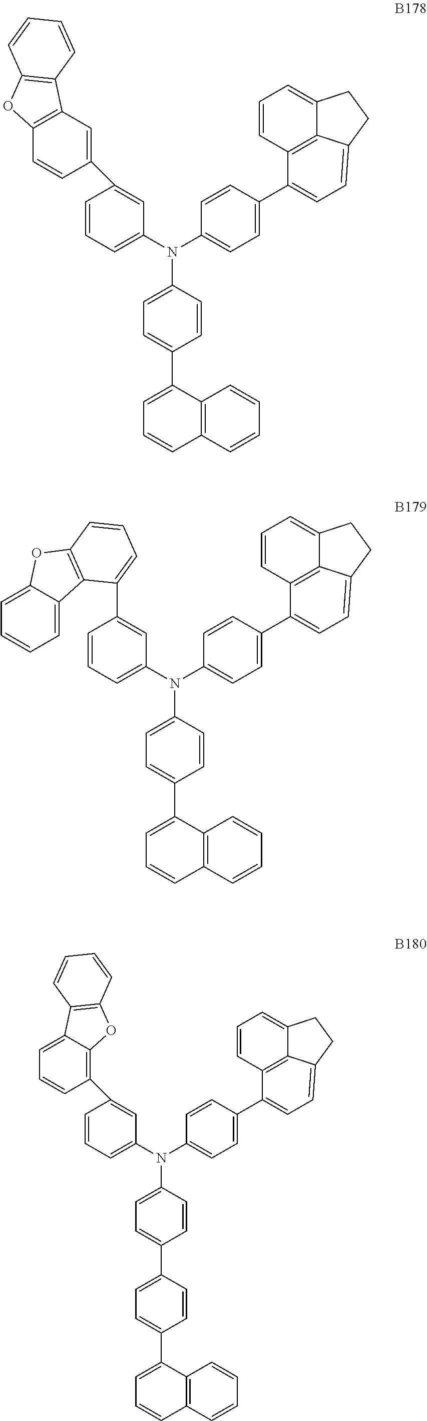

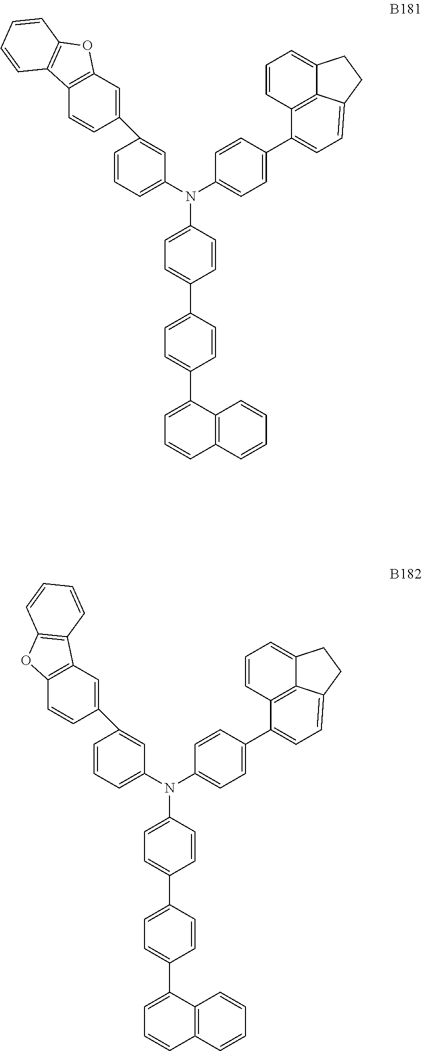

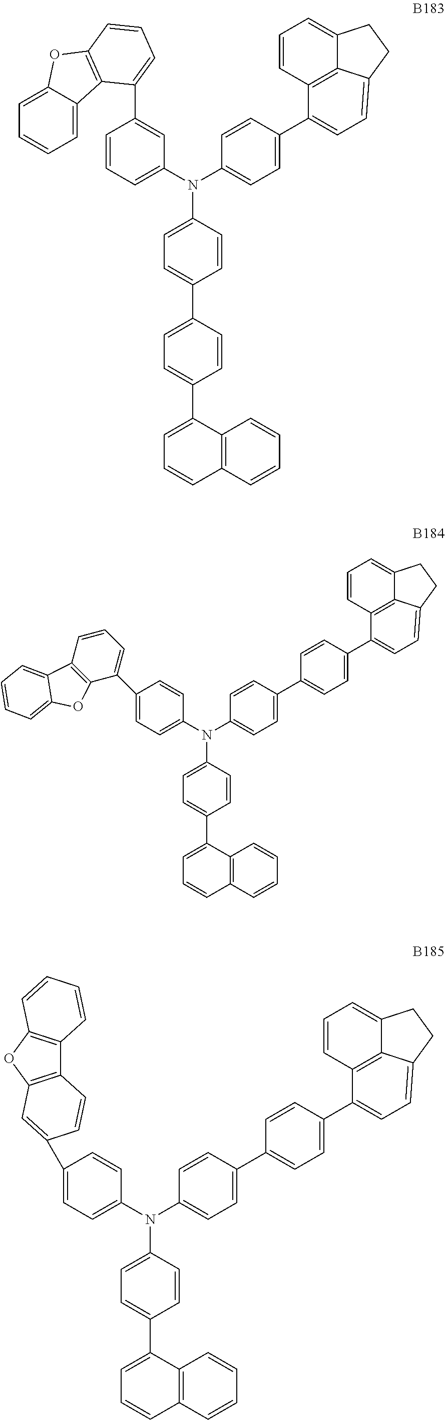

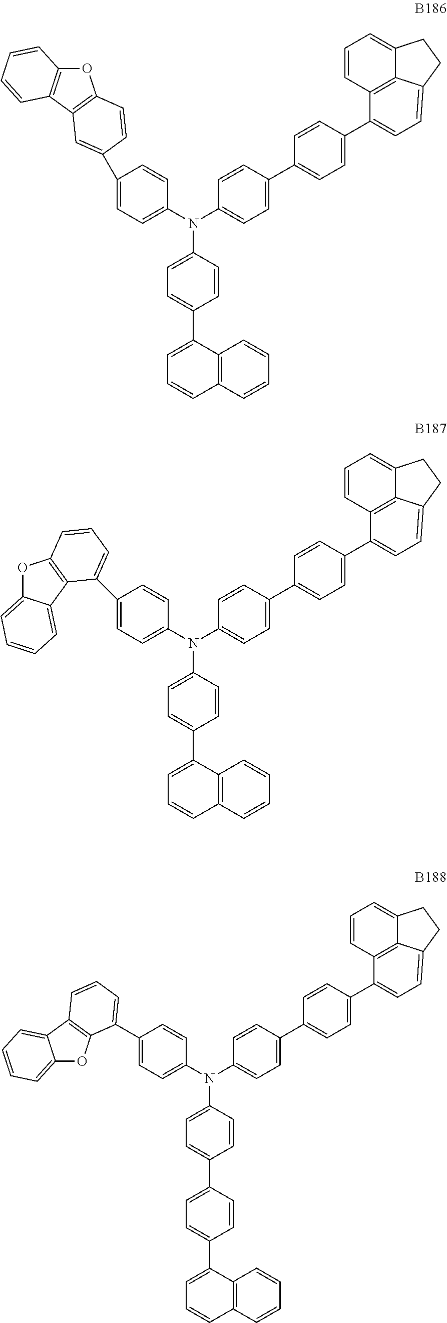

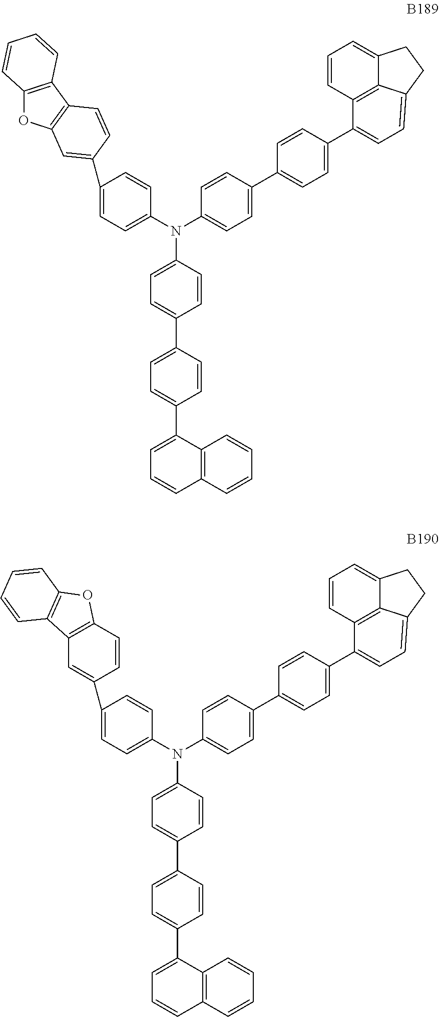

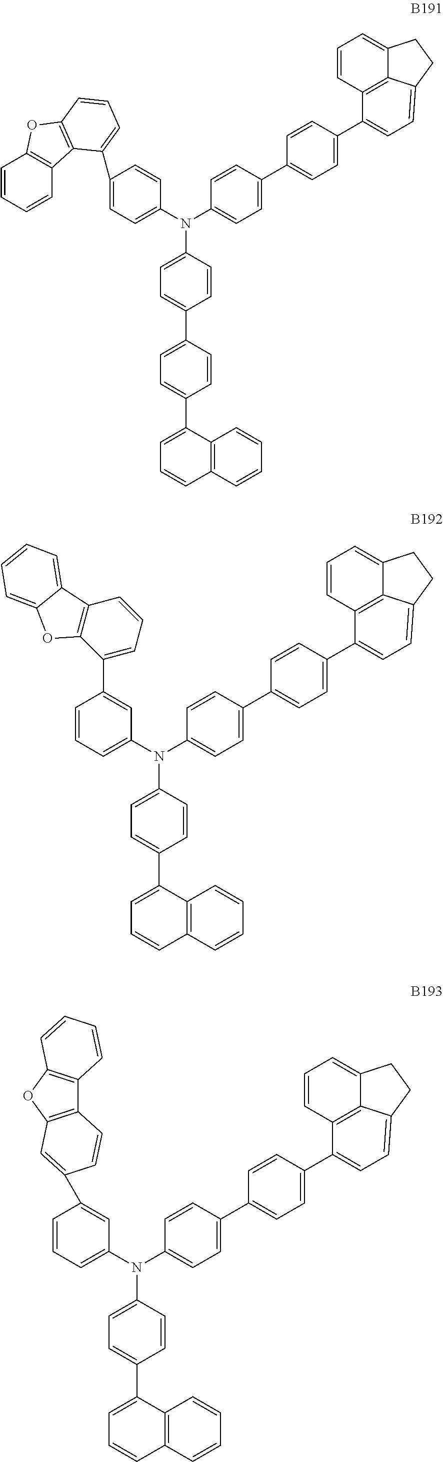

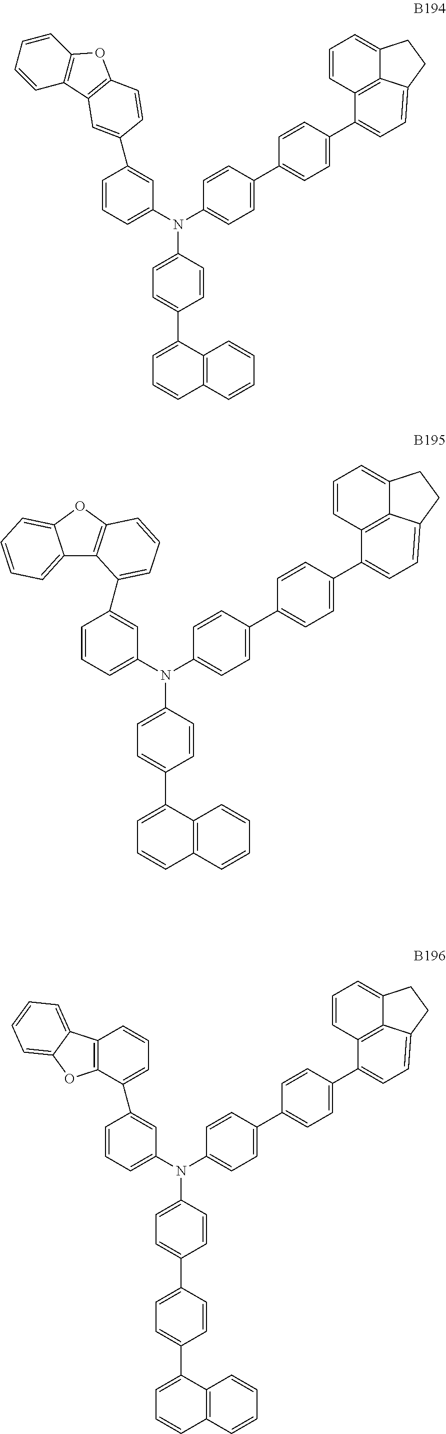

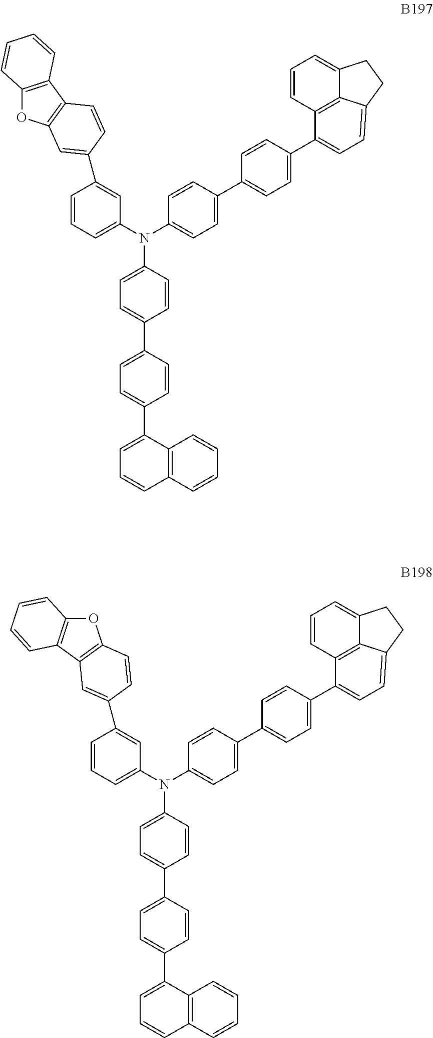

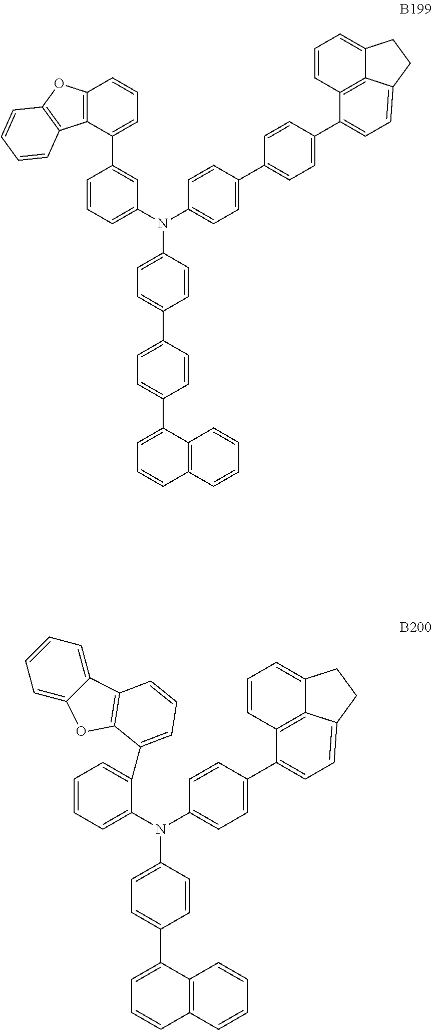

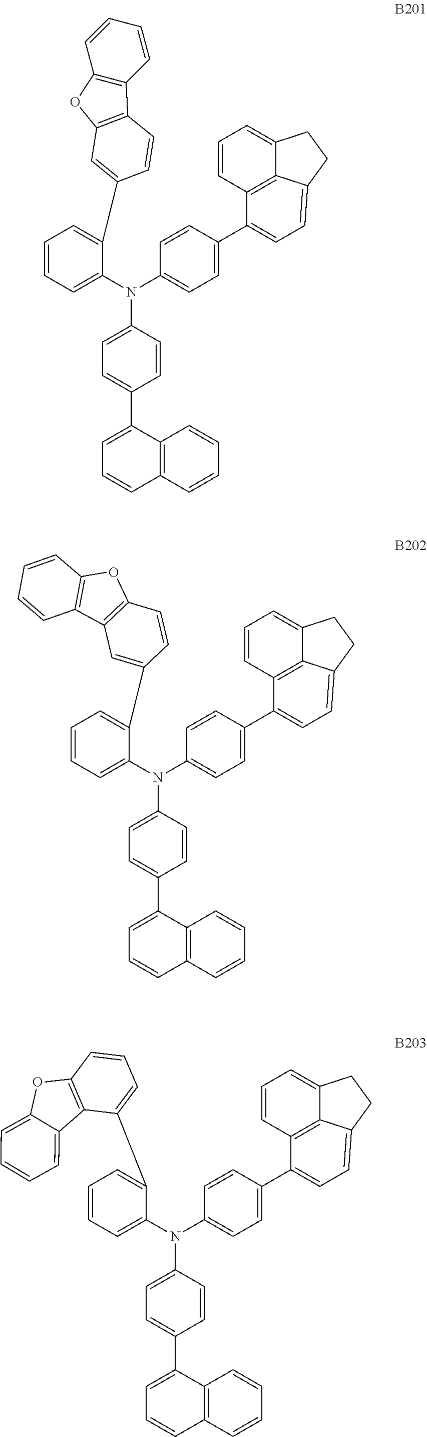

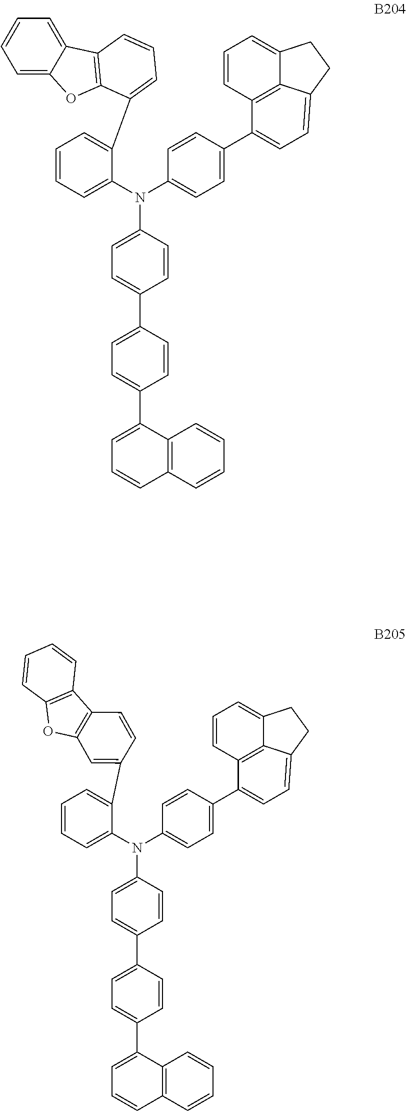

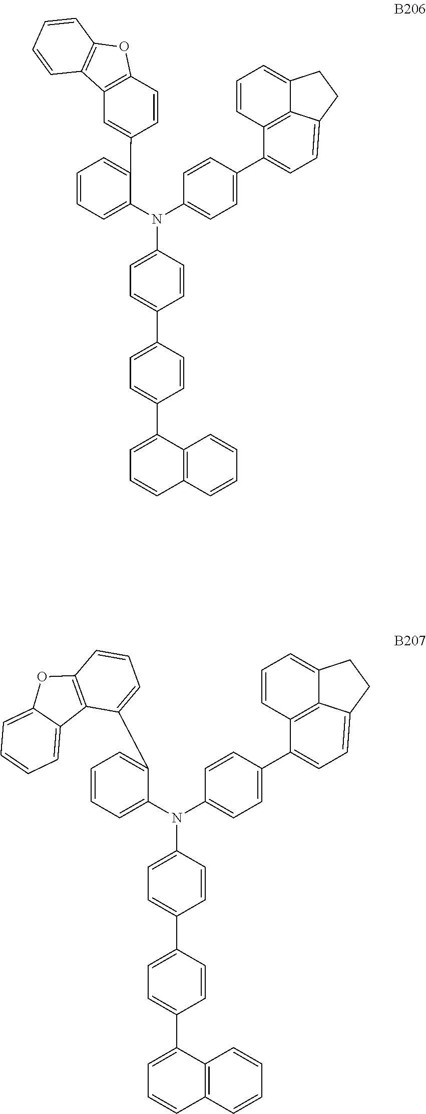

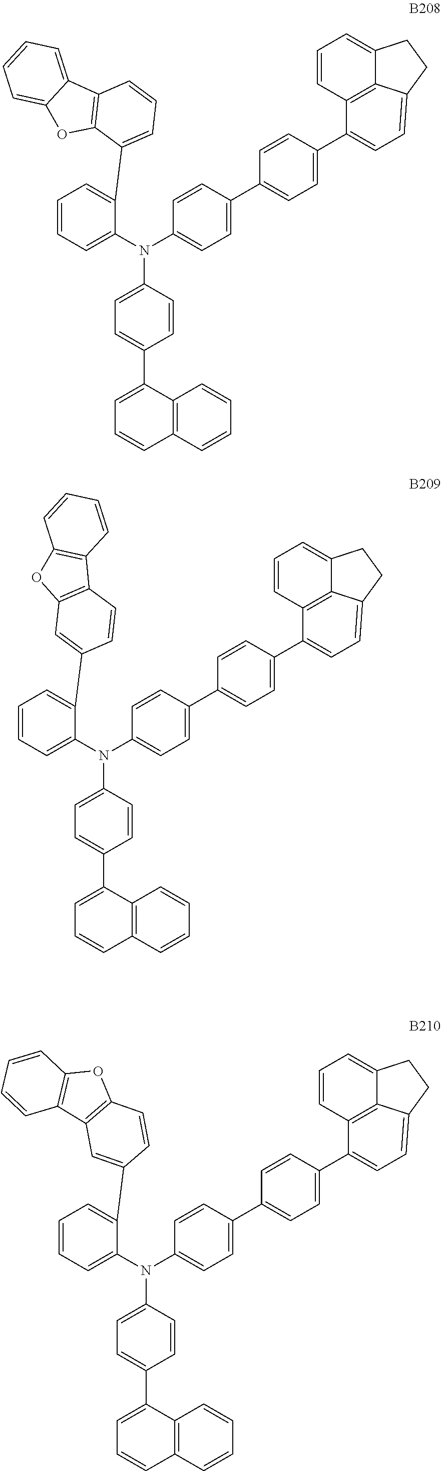

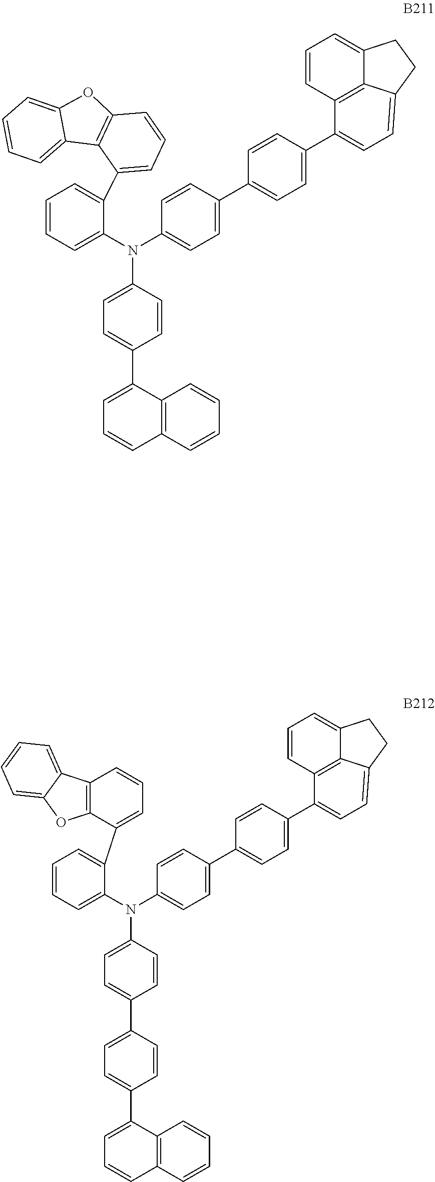

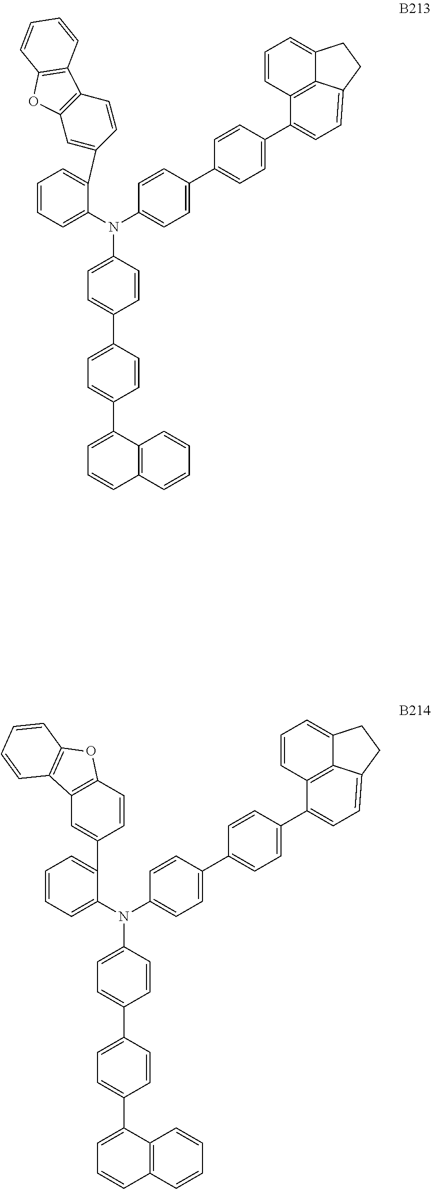

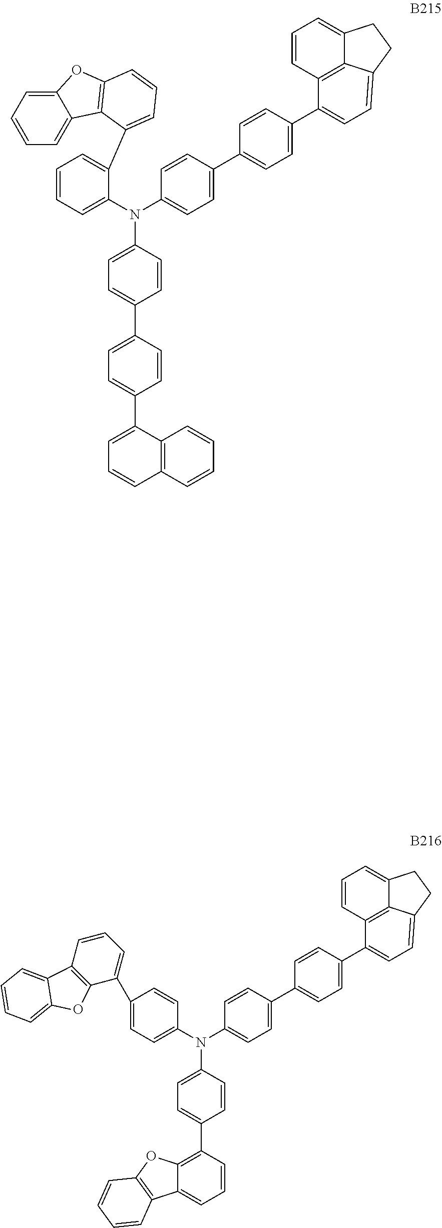

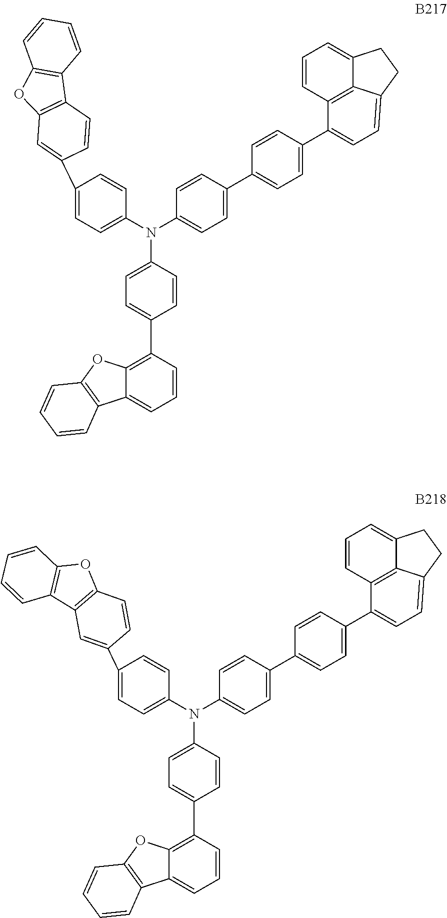

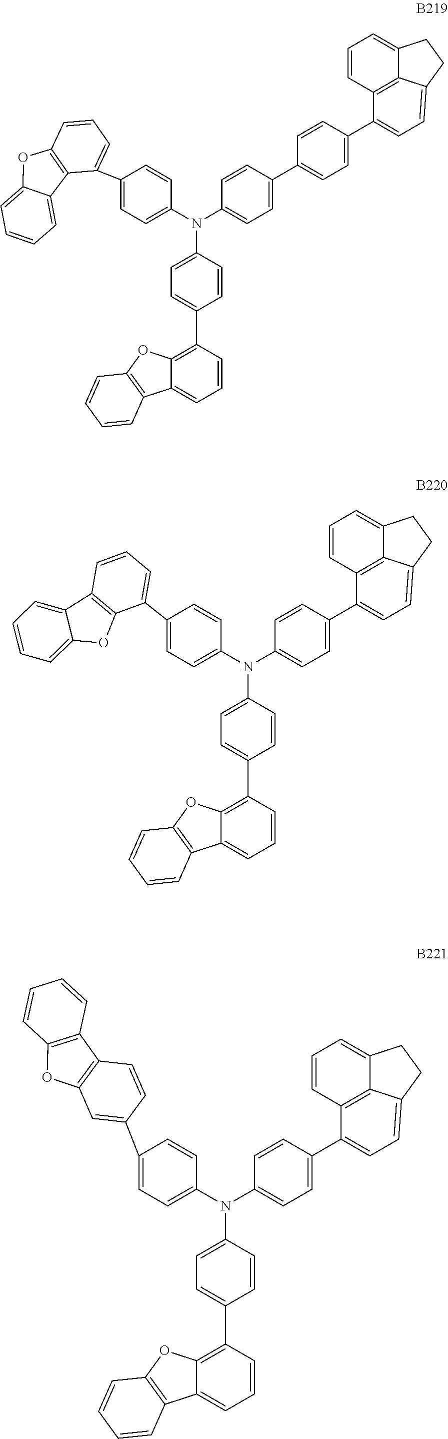

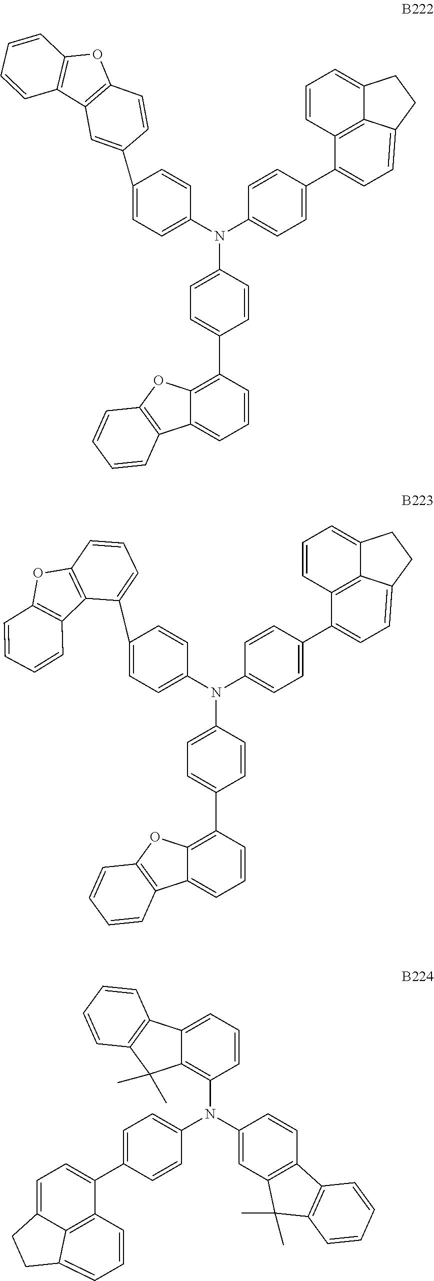

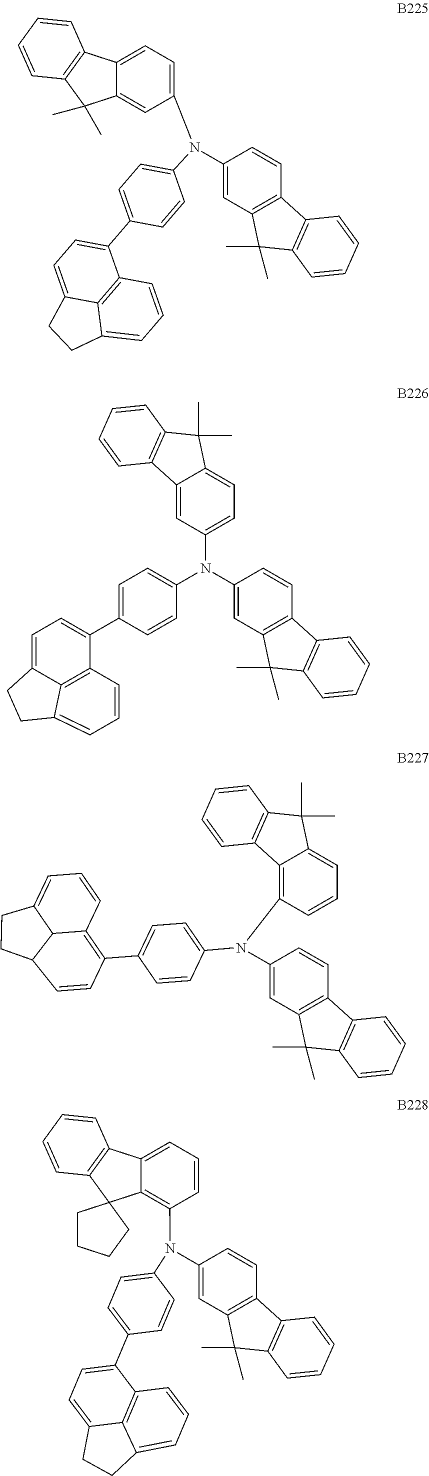

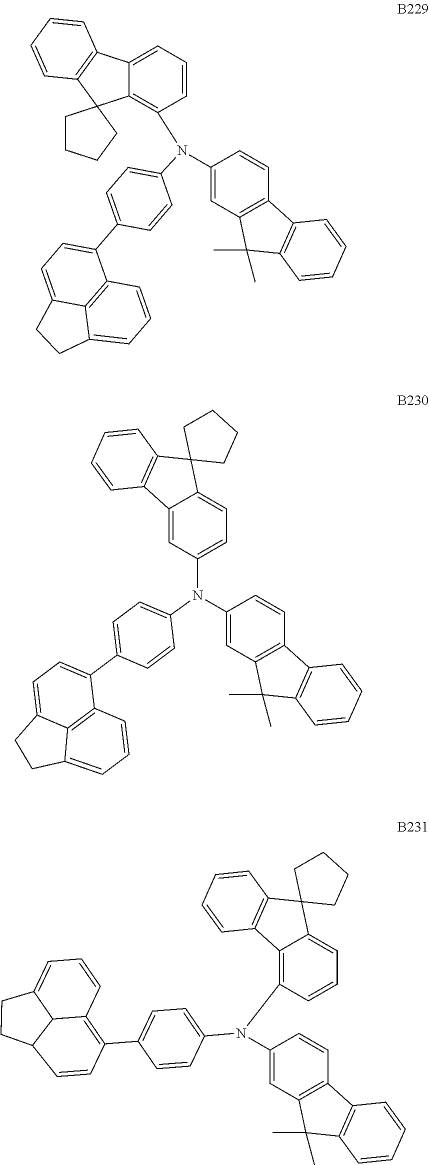

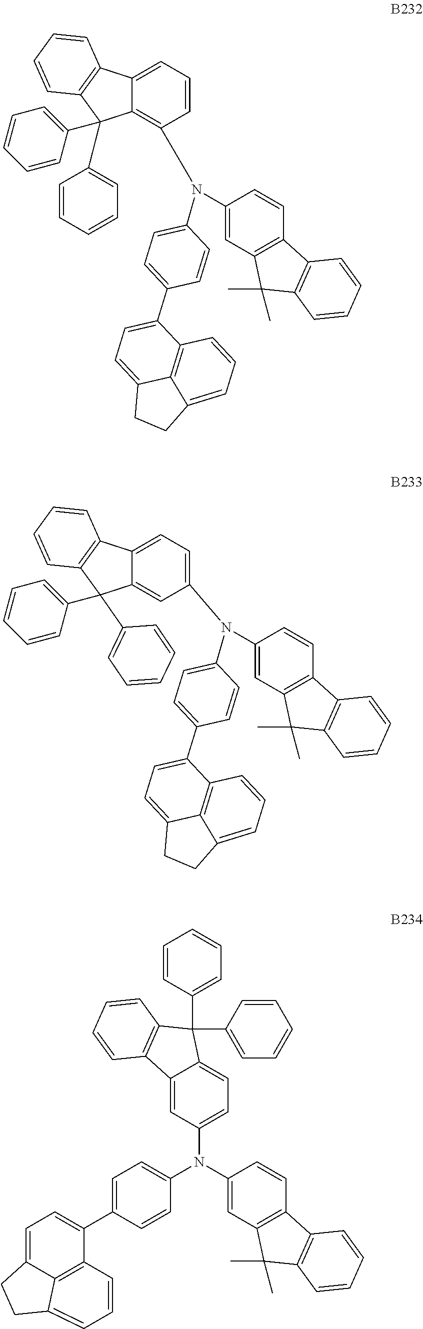

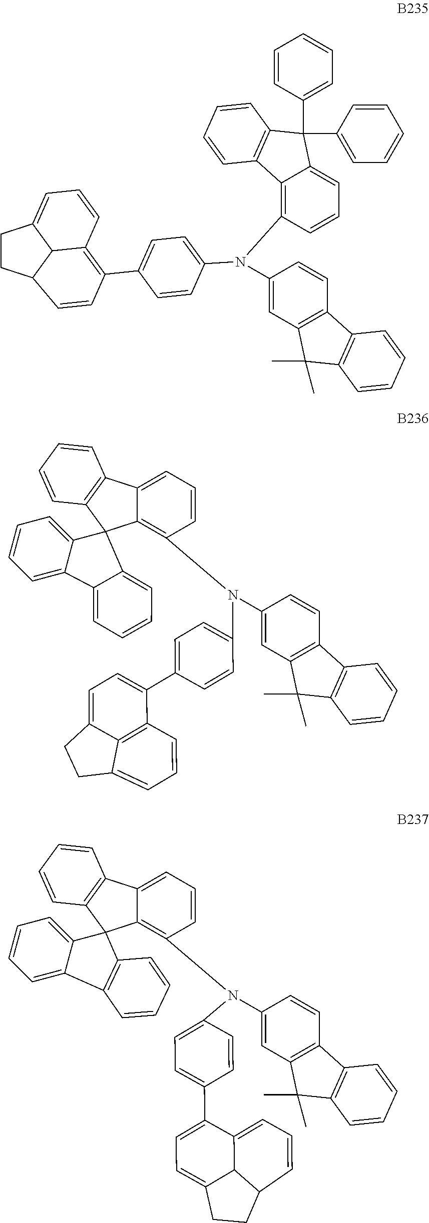

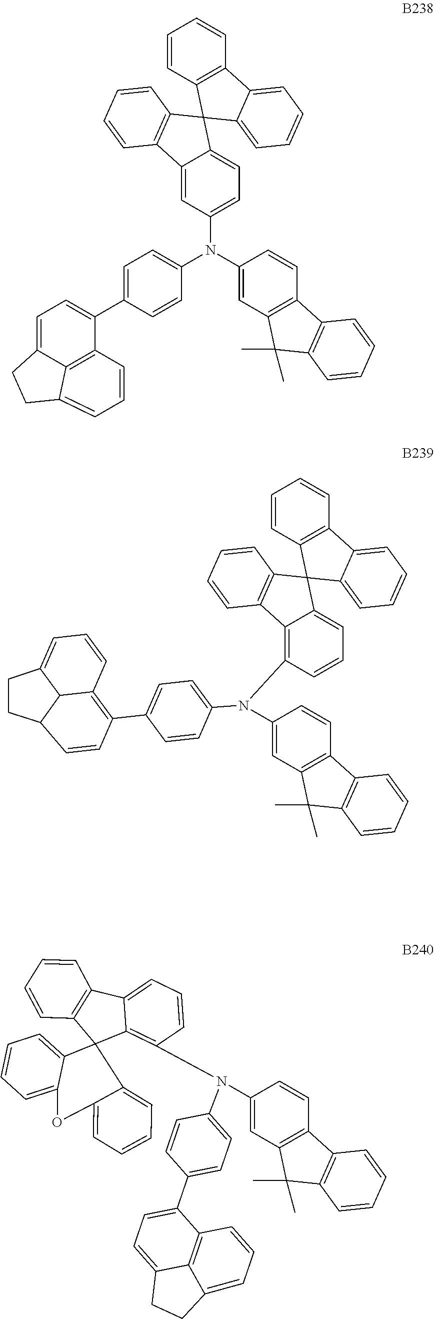

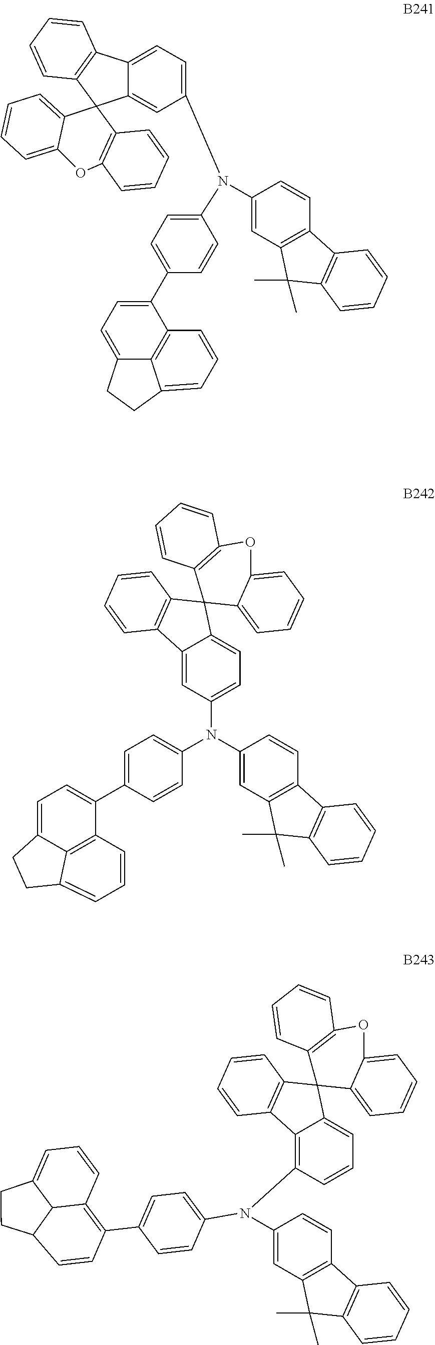

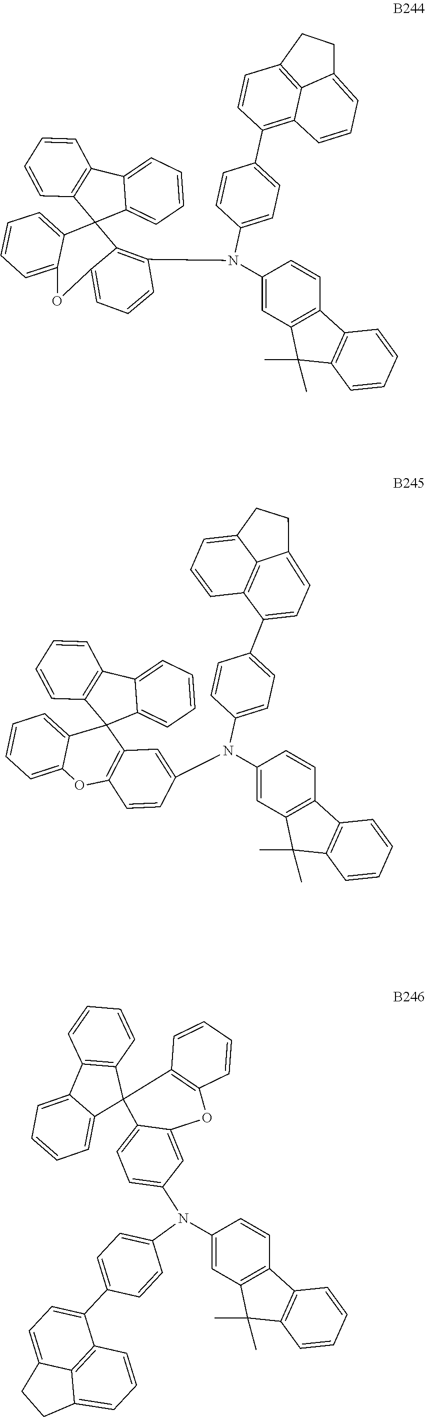

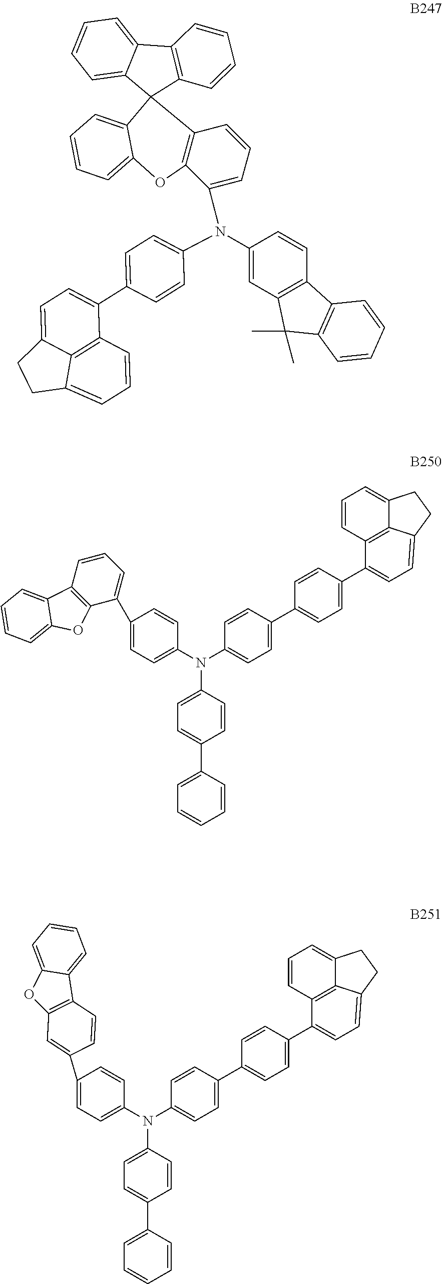

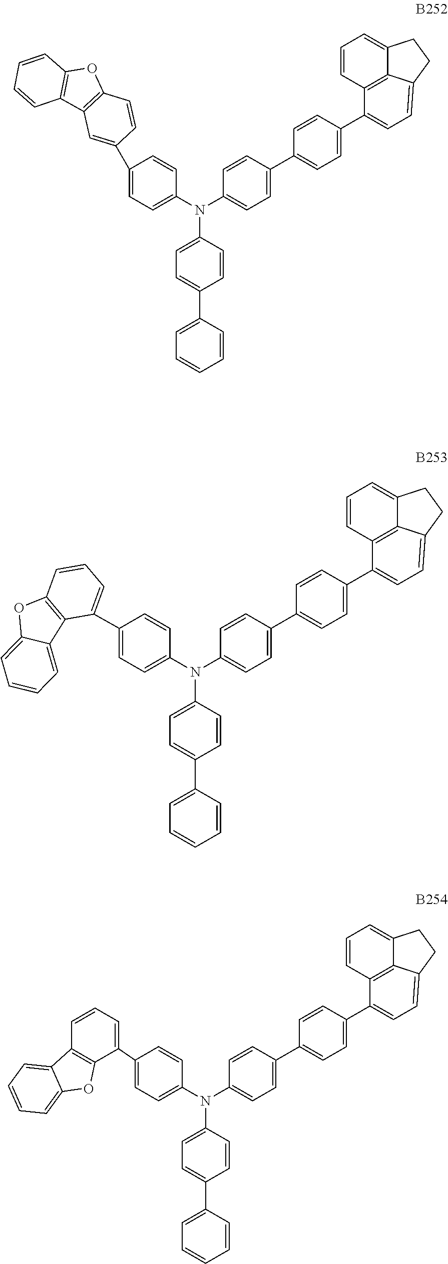

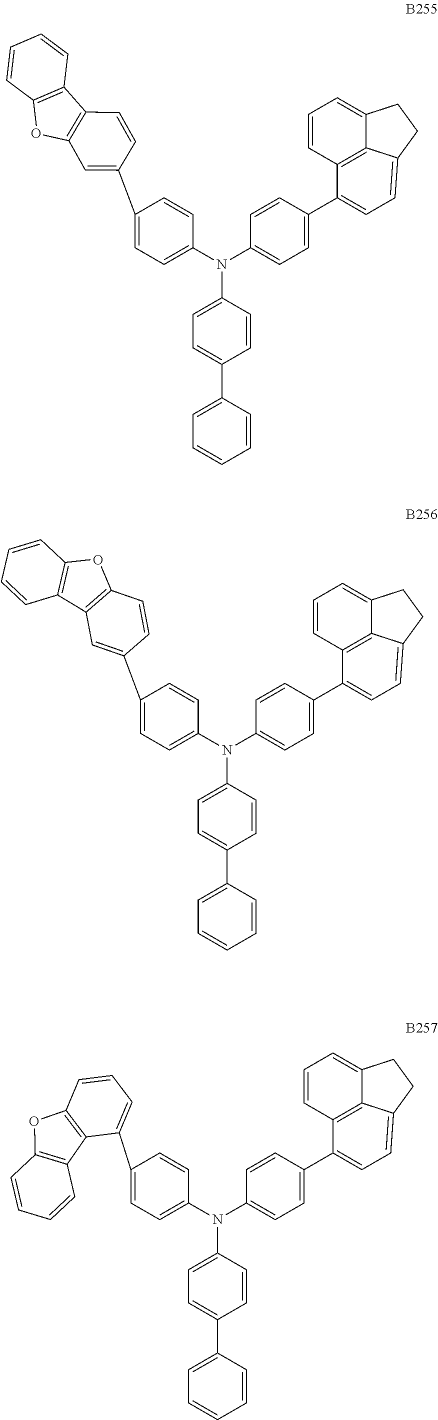

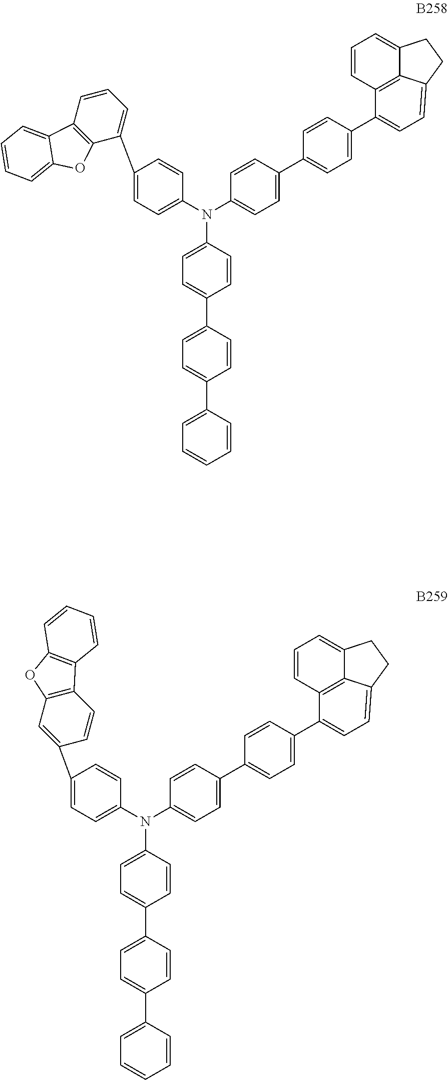

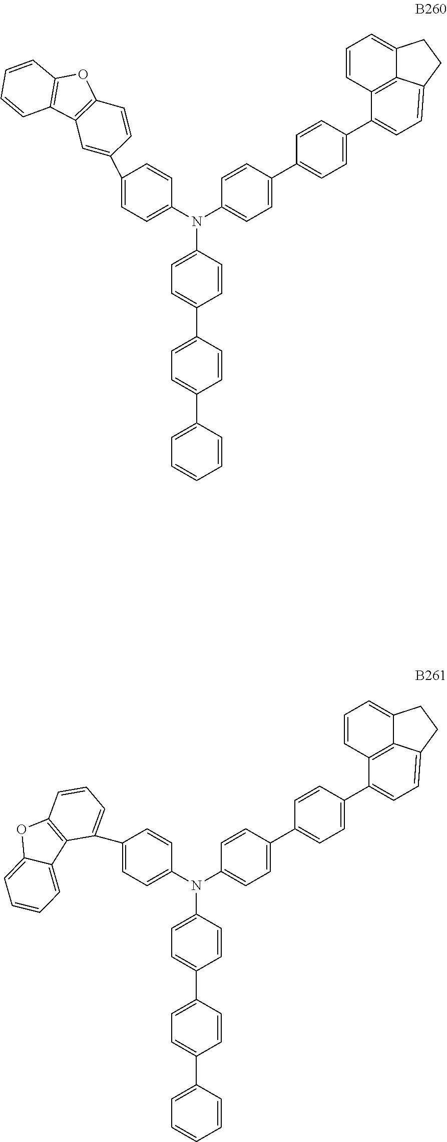

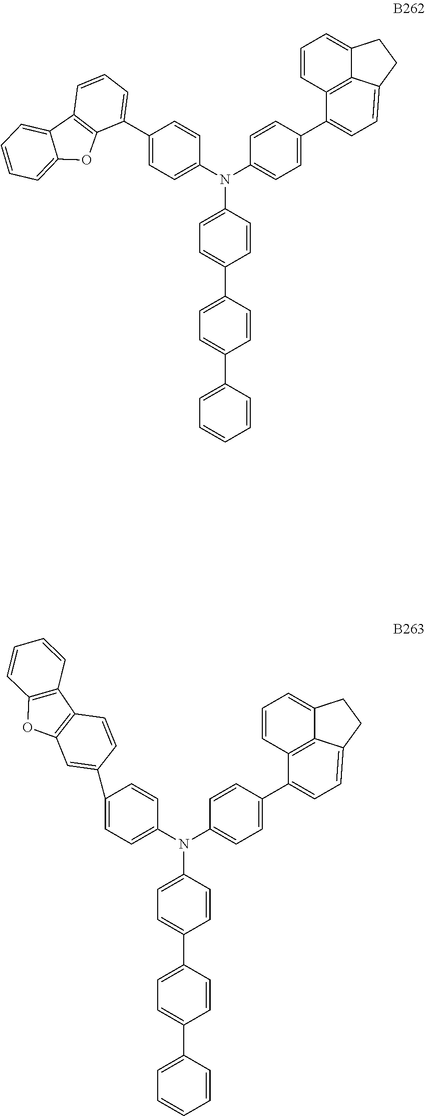

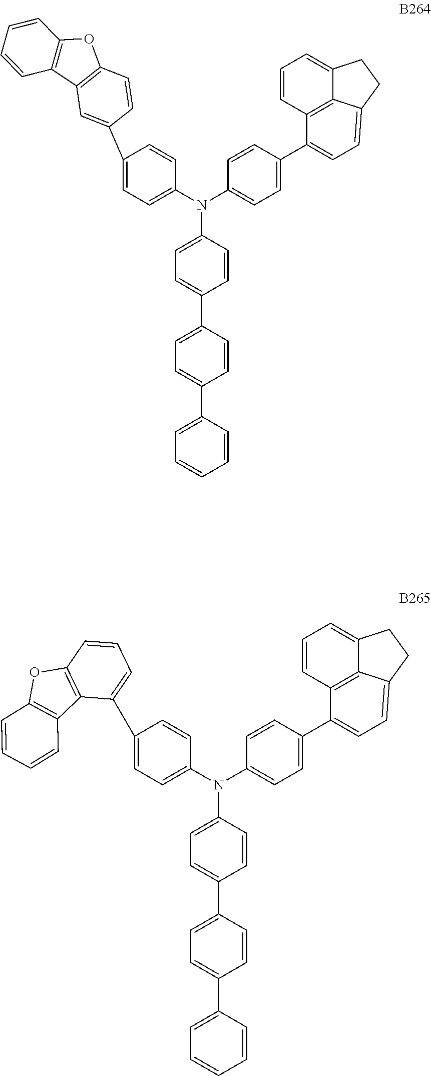

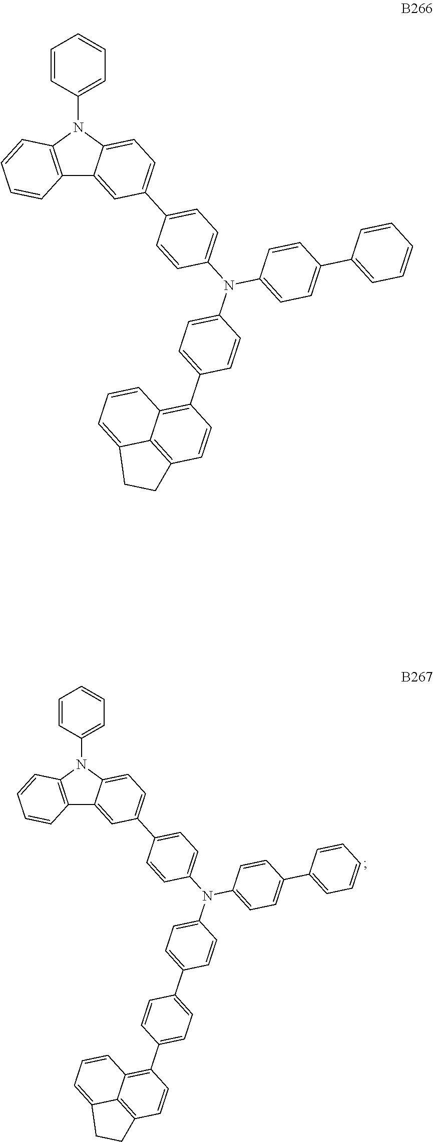

wherein, the substituted compound W4 is formed by replacing one or more hydrogens on the unsubstituted compound W4 with deuterium.

In an example, the substituted compound W4 is formed by replacing all hydrogens on the unsubstituted compound W4 with deuterium.

In another example, the substituted compound W4 is formed by replacing all hydrogens on SP²-hybridized carbons of the unsubstituted compound W4 with deuterium.

In some embodiments of the present disclosure, a third type of aromatic amine compounds is provided. The structural formula of the third type of aromatic amine compounds is shown in Chemical Formula II:

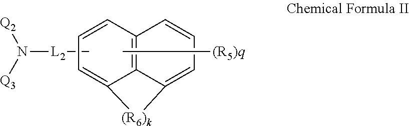

wherein, L₂ is selected from a single bond, a substituted or unsubstituted aryl group having 6 to 30 carbon atoms, or a substituted or unsubstituted heteroaryl group having 3 to 30 carbon atoms; Q₂ and Q₃ are each independently selected from a substituted or unsubstituted aryl group having 6 to 50 carbon atoms or a substituted or unsubstituted heteroaryl group having 3 to 50 carbon atoms, with at least one of Q₂ or Q₃ containing a fused heteroaromatic ring; the heteroatoms in the fused heteroaromatic ring are independently selected from N, O, and S.

The third type of aromatic amine compounds exhibits high refractive index and excellent thermal stability, making it suitable for application as cover layers in electroluminescent devices to enhance their performance. These compounds feature an aromatic amine core structure with fused heteroaromatic rings (containing heteroatoms), which increases their polarizability. The introduction of group P6 further enhances the refractive index of these compounds, thereby improving the light extraction efficiency of the electroluminescent devices. Moreover, the incorporation of group P6 and fused heteroaromatic rings enables the third type of aromatic amine compounds to achieve a refractive index greater than 1.8 with low absorption intensity (less than 0.1) in the wavelength range of 450 to 630 nm, while maintaining strong absorption at 400 nm. This not only improves the light extraction efficiency for visible light but also absorbs ultraviolet (UV) light, thereby extending the device lifetime. In this example, at least one of Q₂ and Q₃ contains a fused heteroaromatic ring, which reduces the dihedral angle between aromatic groups in the third type of aromatic amine compounds, enhances planarity, and strengthens conjugation. This contributes to an increased refractive index and stronger absorption at 400 nm, improving UV light absorption. Additionally, the introduction of group P6 ensures that these compounds maintain a high refractive index while moderately increasing structural steric hindrance, thereby improving film-forming properties (e.g., raising the glass transition temperature).

In an embodiment of the third type of aromatic amine compounds, at least one of Q₂ or Q₃ contains the following fused heteroaromatic ring groups:

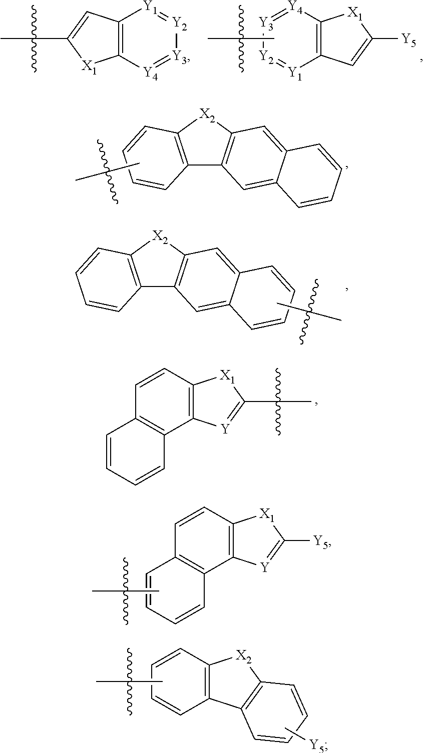

wherein, X₁ is selected from C(Y₆Y₇), NY₈, O, or S; X₂ is selected from NY₈, O, or S;
Y₁, Y₂, Y₃, and Y₄ are each independently selected from CY₈ or N, with only one of them being N;
Y₅, Y₆, Y₇, and Y₈ are each independently selected from hydrogen, deuterium, an alkyl group having 1 to 30 carbon atoms, an arylene group having 6 to 20 carbon atoms, or a heteroaryl group having 5 to 20 carbon atoms.

In an embodiment of the third type of aromatic amine compounds, X₁ is selected from O or S; X₂ is selected from O or S; Y₈ is selected from hydrogen or deuterium; Y₈ is selected from hydrogen, deuterium, phenyl, biphenyl, terphenyl, naphthyl, anthracenyl, or phenanthrenyl.

In an embodiment of the third type of aromatic amine compounds, at least one of Q₂ or Q₃ is selected from the group consisting of the following substituents:

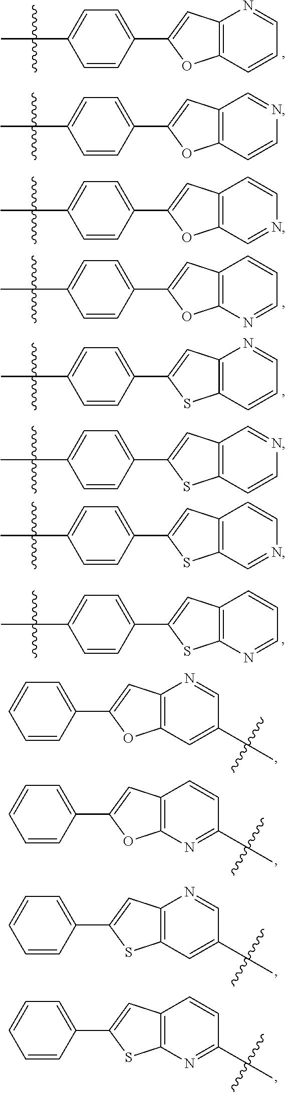

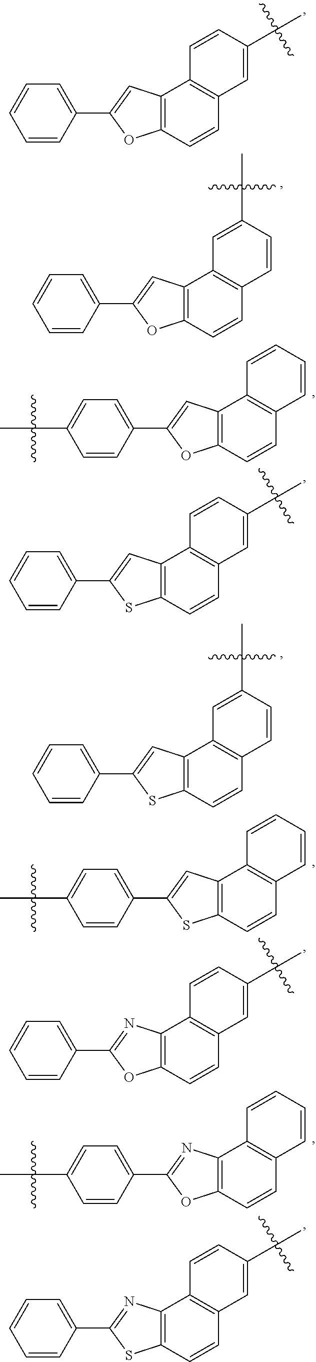

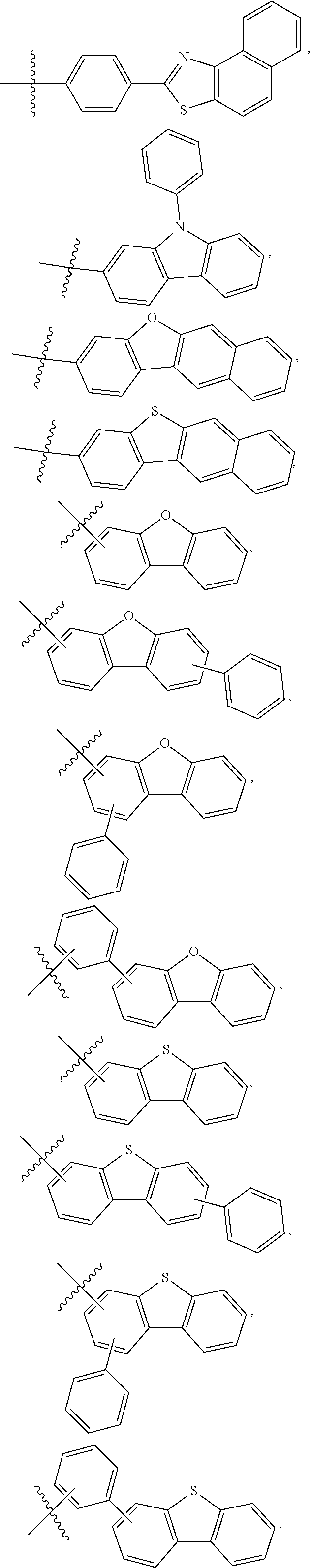

In an embodiment of the third type of aromatic amine compounds, when Q₂ does not contain a fused heteroaromatic ring, Q₂ is selected from a substituted or unsubstituted group W5; when Q₃ does not contain a fused heteroaromatic ring, Q₃ is selected from a substituted or unsubstituted group W5;
the unsubstituted group W5 is selected from an aryl group having 6 to 40 carbon atoms or a heteroaryl group having 5 to 40 carbon atoms;
wherein, the substituted group W5 is formed by replacing one or more hydrogens on the unsubstituted group W5 with substituents selected from deuterium, fluorine, chlorine, bromine, an alkyl group having 1 to 6 carbon atoms, a cycloalkyl group having 6 to 10 carbon atoms, or an aryl group having 6 to 12 carbon atoms; when the number of substituents on L2 is plural, any two substituents are the same or different.

In another embodiment of the third type of aromatic amine compounds, the unsubstituted group W5 is selected from biphenyl, terphenyl, naphthyl, naphthyl-substituted phenyl, naphthyl-substituted biphenyl, anthracenyl, anthracenyl-substituted phenyl, anthracenyl-substituted biphenyl, phenanthrenyl, phenanthrenyl-substituted phenyl, or phenanthrenyl-substituted biphenyl.

In an embodiment of the third type of aromatic amine compounds, L₂ is selected from phenylene, biphenylene, terphenylene, naphthylene, dibenzofuranylene, or dibenzothienylene. In an example, L₂ is phenylene.

In an embodiment of the third type of aromatic amine compounds, the aromatic amine compounds are selected from the group consisting of the following compounds:

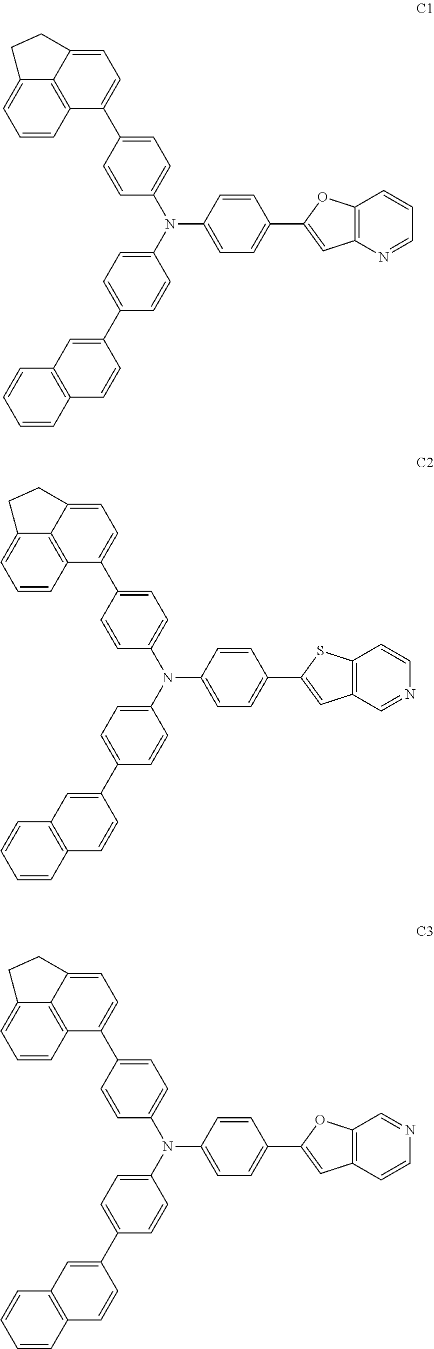 [SMILES: c1ccc2cc(-c3ccc(N(c4ccc(-c5cc6ncccc6o5)cc4)c4ccc(-c5ccc6c7c(cccc57)CC6)cc4)cc3)ccc2c1 ; c1ccc2cc(-c3ccc(N(c4ccc(-c5cc6cnccc6s5)cc4)c4ccc(-c5ccc6c7c(cccc57)CC6)cc4)cc3)ccc2c1 ; c1ccc2cc(-c3ccc(N(c4ccc(-c5cc6ccncc6o5)cc4)c4ccc(-c5ccc6c7c(cccc57)CC6)cc4)cc3)ccc2c1]

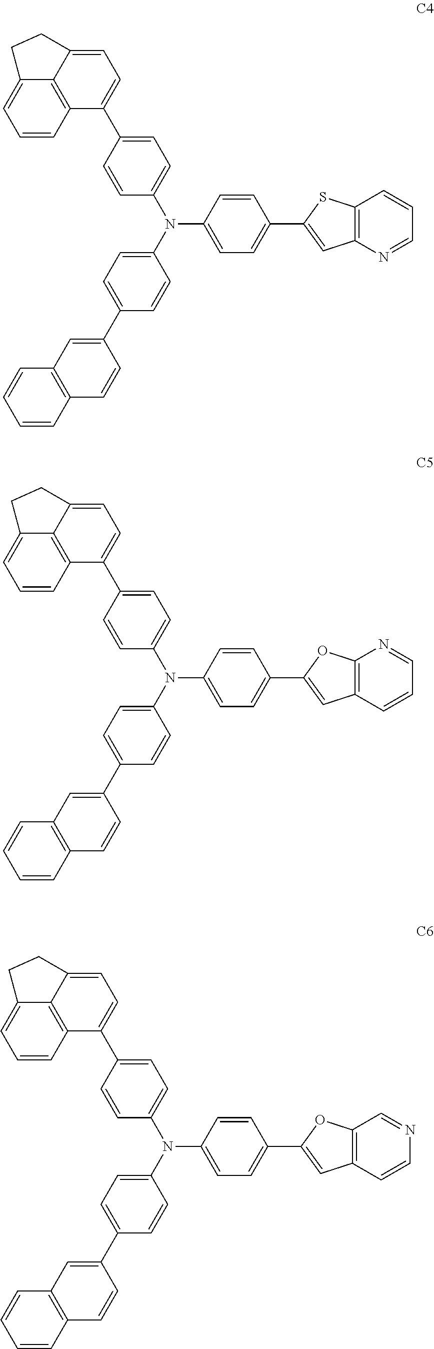 [SMILES: c1ccc2cc(-c3ccc(N(c4ccc(-c5cc6ncccc6s5)cc4)c4ccc(-c5ccc6c7c(cccc57)CC6)cc4)cc3)ccc2c1 ; c1ccc2cc(-c3ccc(N(c4ccc(-c5cc6cccnc6o5)cc4)c4ccc(-c5ccc6c7c(cccc57)CC6)cc4)cc3)ccc2c1 ; c1ccc2cc(-c3ccc(N(c4ccc(-c5cc6ccncc6o5)cc4)c4ccc(-c5ccc6c7c(cccc57)CC6)cc4)cc3)ccc2c1]

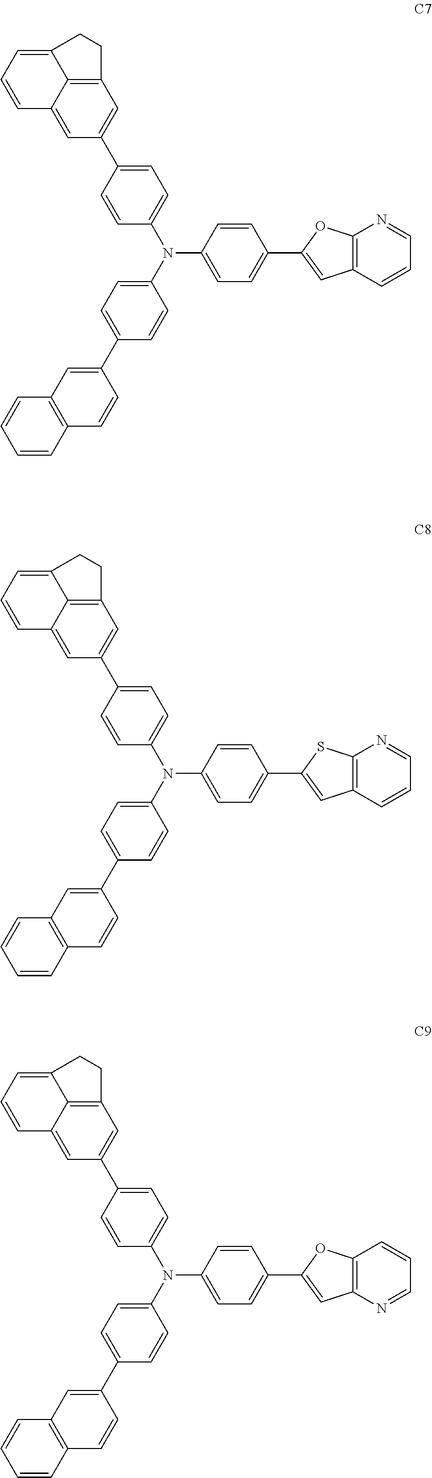 [SMILES: c1ccc2cc(-c3ccc(N(c4ccc(-c5cc6c7c(cccc7c5)CC6)cc4)c4ccc(-c5cc6cccnc6o5)cc4)cc3)ccc2c1 ; c1ccc2cc(-c3ccc(N(c4ccc(-c5cc6c7c(cccc7c5)CC6)cc4)c4ccc(-c5cc6cccnc6s5)cc4)cc3)ccc2c1 ; c1ccc2cc(-c3ccc(N(c4ccc(-c5cc6c7c(cccc7c5)CC6)cc4)c4ccc(-c5cc6ncccc6o5)cc4)cc3)ccc2c1]

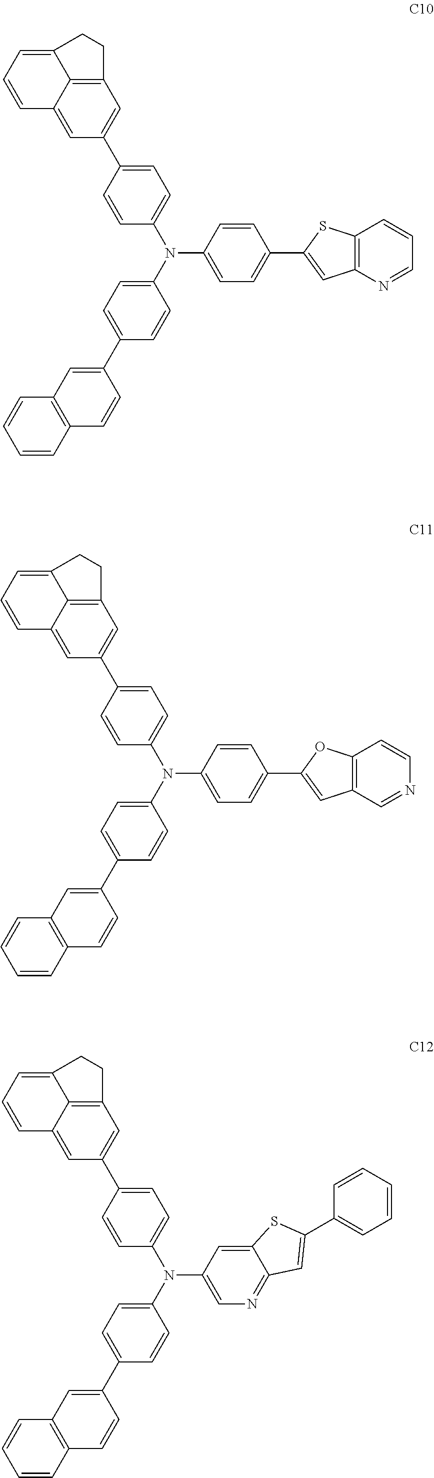 [SMILES: c1ccc2cc(-c3ccc(N(c4ccc(-c5cc6c7c(cccc7c5)CC6)cc4)c4ccc(-c5cc6ncccc6s5)cc4)cc3)ccc2c1 ; c1ccc2cc(-c3ccc(N(c4ccc(-c5cc6c7c(cccc7c5)CC6)cc4)c4ccc(-c5cc6cnccc6o5)cc4)cc3)ccc2c1 ; c1ccc(-c2cc3ncc(N(c4ccc(-c5ccc6ccccc6c5)cc4)c4ccc(-c5cc6c7c(cccc7c5)CC6)cc4)cc3s2)cc1]

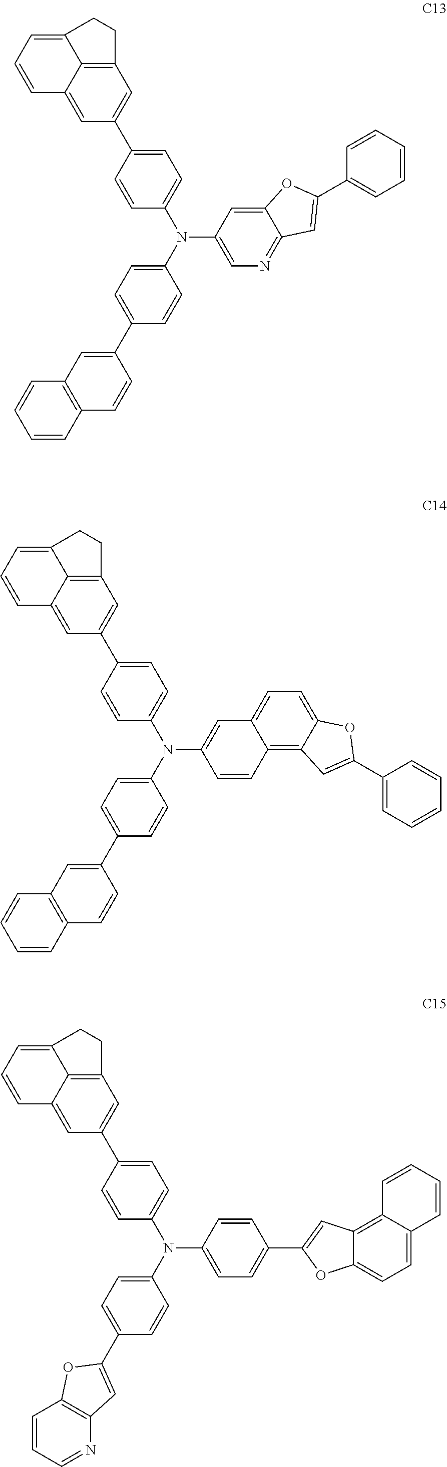 [SMILES: c1ccc(-c2cc3ncc(N(c4ccc(-c5ccc6ccccc6c5)cc4)c4ccc(-c5cc6c7c(cccc7c5)CC6)cc4)cc3o2)cc1 ; c1ccc(-c2cc3c(ccc4cc(N(c5ccc(-c6ccc7ccccc7c6)cc5)c5ccc(-c6cc7c8c(cccc8c6)CC7)cc5)ccc43)o2)cc1 ; c1cc2c3c(cc(-c4ccc(N(c5ccc(-c6cc7ncccc7o6)cc5)c5ccc(-c6cc7c(ccc8ccccc87)o6)cc5)cc4)cc3c1)CC2]

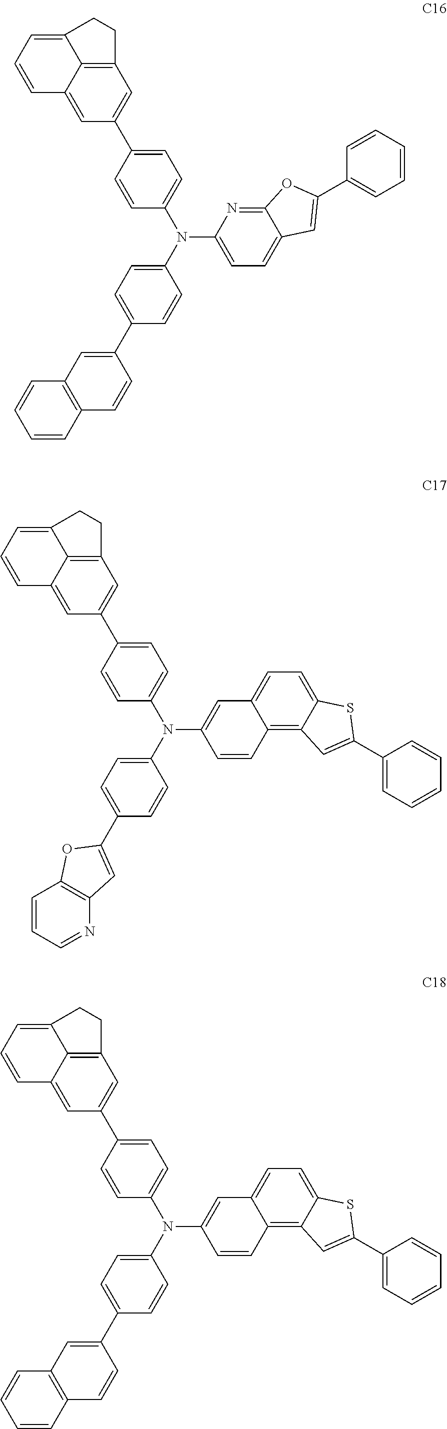 [SMILES: c1ccc(-c2cc3ccc(N(c4ccc(-c5ccc6ccccc6c5)cc4)c4ccc(-c5cc6c7c(cccc7c5)CC6)cc4)nc3o2)cc1 ; c1ccc(-c2cc3c(ccc4cc(N(c5ccc(-c6cc7c8c(cccc8c6)CC7)cc5)c5ccc(-c6cc7ncccc7o6)cc5)ccc43)s2)cc1 ; c1ccc(-c2cc3c(ccc4cc(N(c5ccc(-c6ccc7ccccc7c6)cc5)c5ccc(-c6cc7c8c(cccc8c6)CC7)cc5)ccc43)s2)cc1]

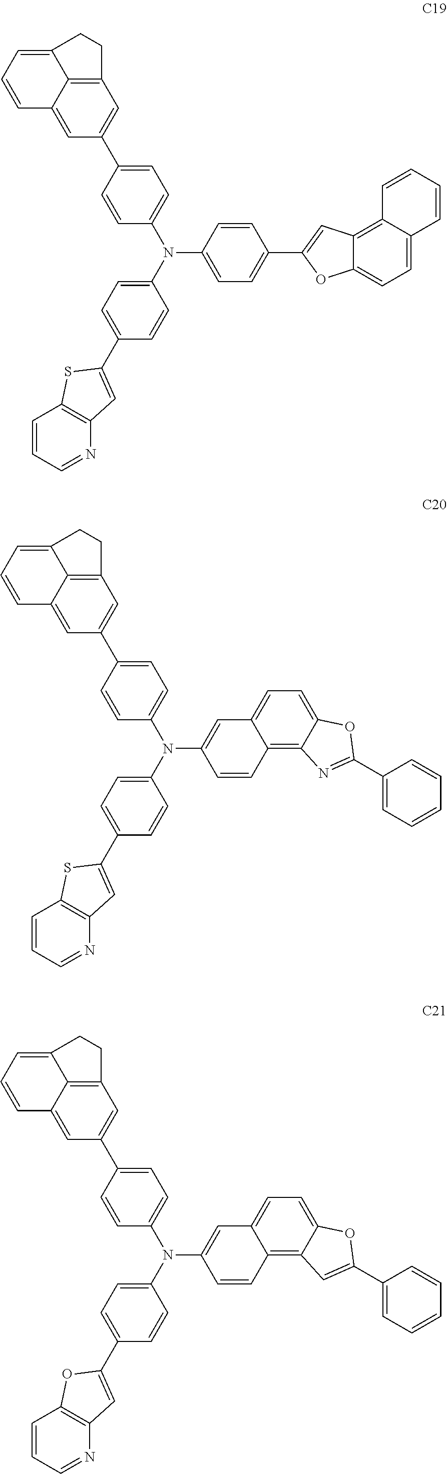 [SMILES: c1cc2c3c(cc(-c4ccc(N(c5ccc(-c6cc7c(ccc8ccccc87)o6)cc5)c5ccc(-c6cc7ncccc7s6)cc5)cc4)cc3c1)CC2 ; c1ccc(-c2nc3c(ccc4cc(N(c5ccc(-c6cc7c8c(cccc8c6)CC7)cc5)c5ccc(-c6cc7ncccc7s6)cc5)ccc43)o2)cc1 ; c1ccc(-c2cc3c(ccc4cc(N(c5ccc(-c6cc7c8c(cccc8c6)CC7)cc5)c5ccc(-c6cc7ncccc7o6)cc5)ccc43)o2)cc1]

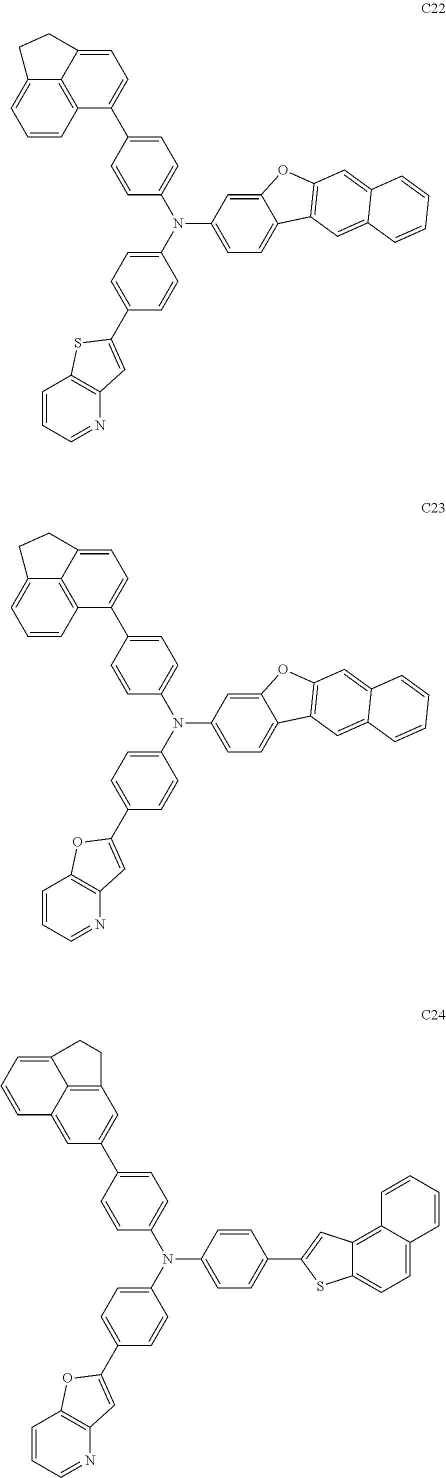 [SMILES: c1ccc2cc3c(cc2c1)oc1cc(N(c2ccc(-c4cc5ncccc5s4)cc2)c2ccc(-c4ccc5c6c(cccc46)CC5)cc2)ccc13 ; c1ccc2cc3c(cc2c1)oc1cc(N(c2ccc(-c4cc5ncccc5o4)cc2)c2ccc(-c4ccc5c6c(cccc46)CC5)cc2)ccc13 ; c1cc2c3c(cc(-c4ccc(N(c5ccc(-c6cc7ncccc7o6)cc5)c5ccc(-c6cc7c(ccc8ccccc87)s6)cc5)cc4)cc3c1)CC2]

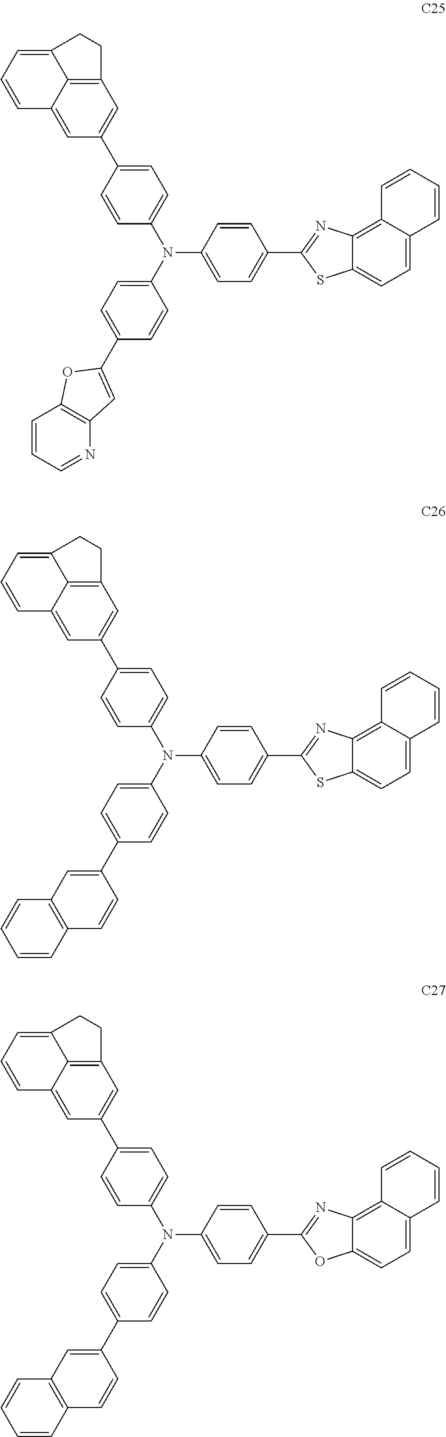 [SMILES: c1cc2c3c(cc(-c4ccc(N(c5ccc(-c6cc7ncccc7o6)cc5)c5ccc(-c6nc7c(ccc8ccccc87)s6)cc5)cc4)cc3c1)CC2 ; c1ccc2cc(-c3ccc(N(c4ccc(-c5cc6c7c(cccc7c5)CC6)cc4)c4ccc(-c5nc6c(ccc7ccccc76)s5)cc4)cc3)ccc2c1 ; c1ccc2cc(-c3ccc(N(c4ccc(-c5cc6c7c(cccc7c5)CC6)cc4)c4ccc(-c5nc6c(ccc7ccccc76)o5)cc4)cc3)ccc2c1]

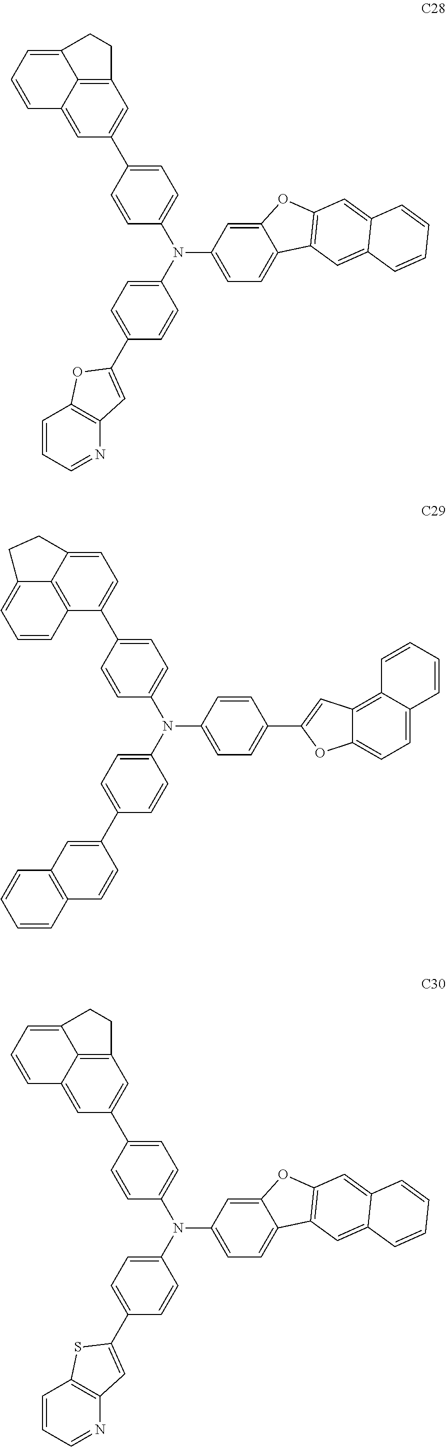 [SMILES: c1ccc2cc3c(cc2c1)oc1cc(N(c2ccc(-c4cc5c6c(cccc6c4)CC5)cc2)c2ccc(-c4cc5ncccc5o4)cc2)ccc13 ; c1ccc2cc(-c3ccc(N(c4ccc(-c5cc6c(ccc7ccccc76)o5)cc4)c4ccc(-c5ccc6c7c(cccc57)CC6)cc4)cc3)ccc2c1 ; c1ccc2cc3c(cc2c1)oc1cc(N(c2ccc(-c4cc5c6c(cccc6c4)CC5)cc2)c2ccc(-c4cc5ncccc5s4)cc2)ccc13]

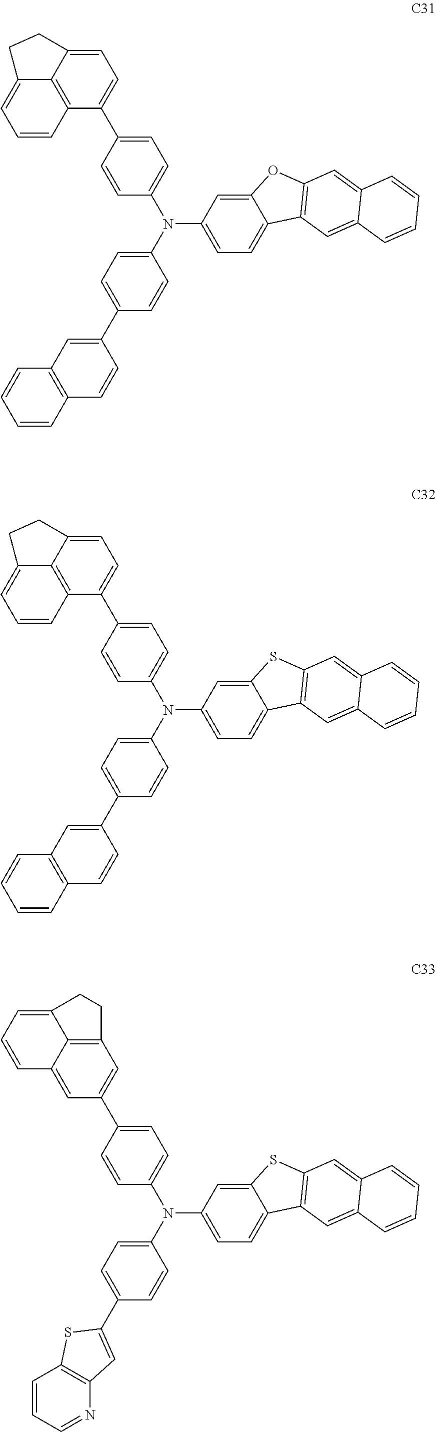 [SMILES: c1ccc2cc(-c3ccc(N(c4ccc(-c5ccc6c7c(cccc57)CC6)cc4)c4ccc5c(c4)oc4cc6ccccc6cc45)cc3)ccc2c1 ; c1ccc2cc(-c3ccc(N(c4ccc(-c5ccc6c7c(cccc57)CC6)cc4)c4ccc5c(c4)sc4cc6ccccc6cc45)cc3)ccc2c1 ; c1ccc2cc3c(cc2c1)sc1cc(N(c2ccc(-c4cc5c6c(cccc6c4)CC5)cc2)c2ccc(-c4cc5ncccc5s4)cc2)ccc13]

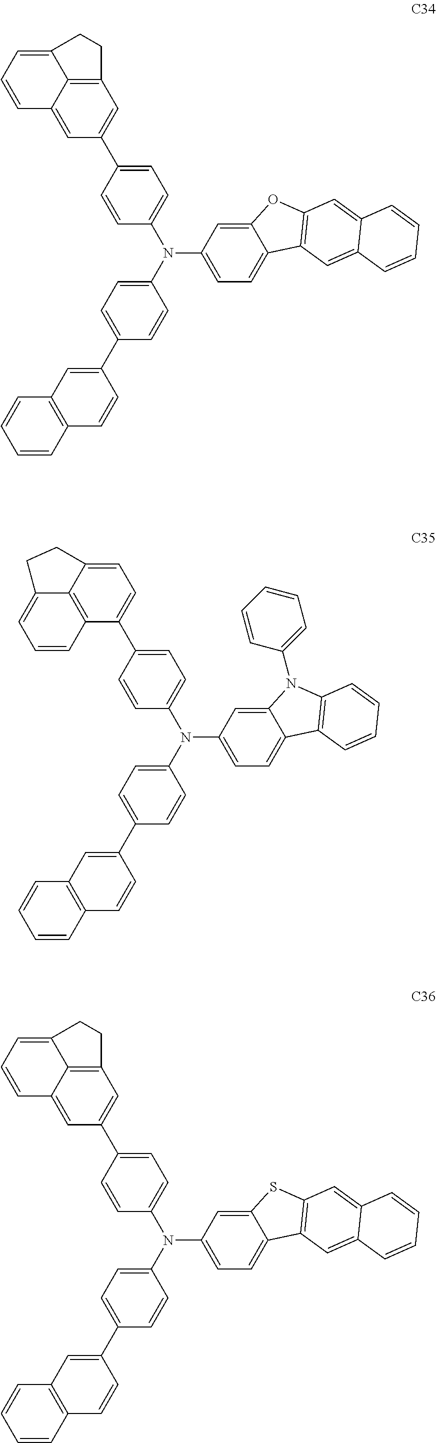 [SMILES: c1ccc2cc(-c3ccc(N(c4ccc(-c5cc6c7c(cccc7c5)CC6)cc4)c4ccc5c(c4)oc4cc6ccccc6cc45)cc3)ccc2c1 ; c1ccc(-n2c3ccccc3c3ccc(N(c4ccc(-c5ccc6ccccc6c5)cc4)c4ccc(-c5ccc6c7c(cccc57)CC6)cc4)cc32)cc1 ; c1ccc2cc(-c3ccc(N(c4ccc(-c5cc6c7c(cccc7c5)CC6)cc4)c4ccc5c(c4)sc4cc6ccccc6cc45)cc3)ccc2c1]

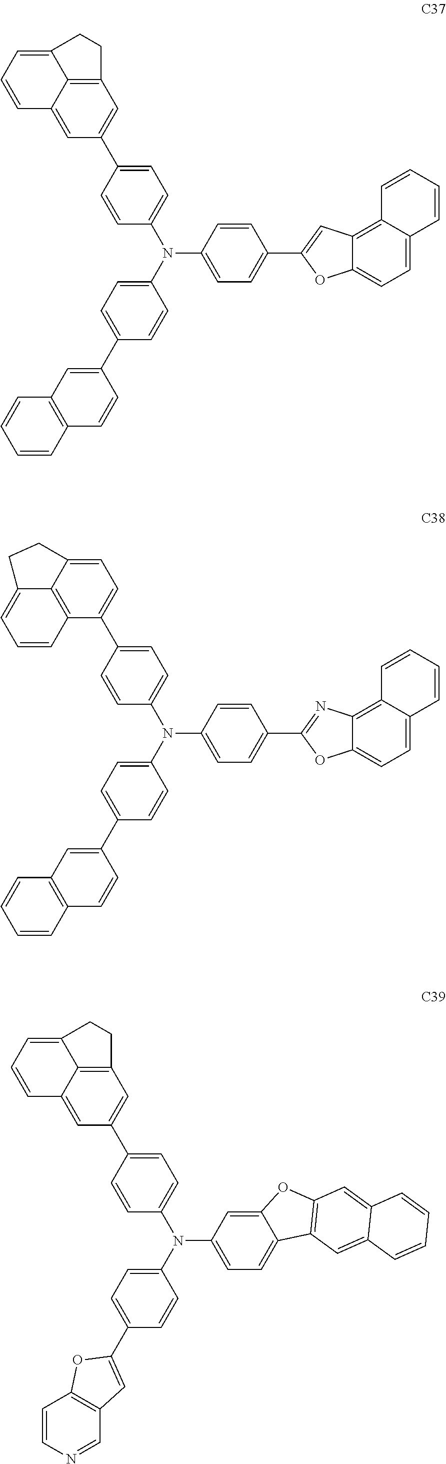 [SMILES: c1ccc2cc(-c3ccc(N(c4ccc(-c5cc6c7c(cccc7c5)CC6)cc4)c4ccc(-c5cc6c(ccc7ccccc76)o5)cc4)cc3)ccc2c1 ; c1ccc2cc(-c3ccc(N(c4ccc(-c5nc6c(ccc7ccccc76)o5)cc4)c4ccc(-c5ccc6c7c(cccc57)CC6)cc4)cc3)ccc2c1 ; c1ccc2cc3c(cc2c1)oc1cc(N(c2ccc(-c4cc5c6c(cccc6c4)CC5)cc2)c2ccc(-c4cc5cnccc5o4)cc2)ccc13]

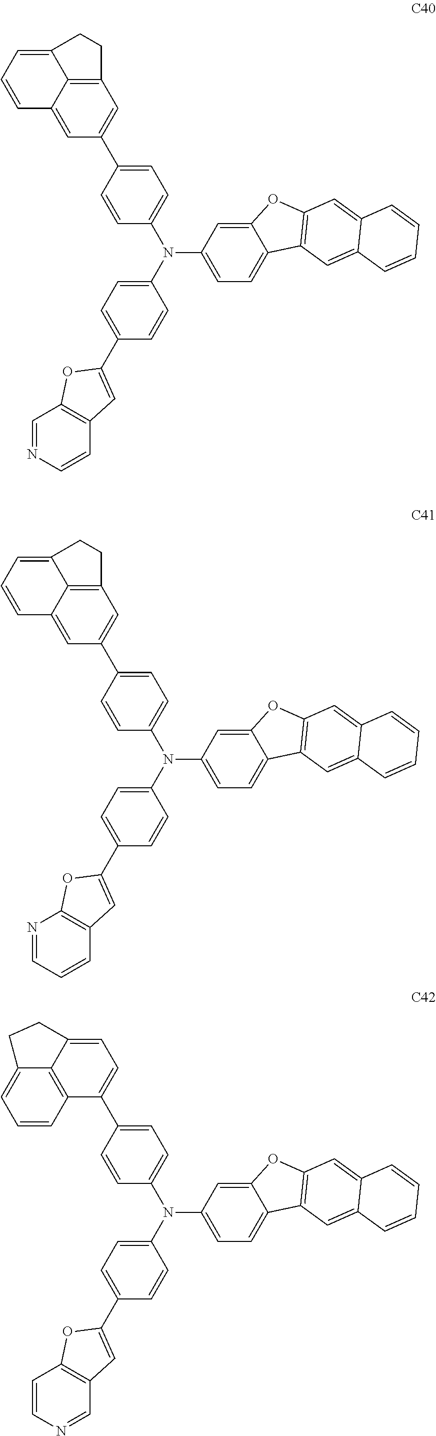 [SMILES: c1ccc2cc3c(cc2c1)oc1cc(N(c2ccc(-c4cc5c6c(cccc6c4)CC5)cc2)c2ccc(-c4cc5ccncc5o4)cc2)ccc13 ; c1ccc2cc3c(cc2c1)oc1cc(N(c2ccc(-c4cc5c6c(cccc6c4)CC5)cc2)c2ccc(-c4cc5cccnc5o4)cc2)ccc13 ; c1ccc2cc3c(cc2c1)oc1cc(N(c2ccc(-c4cc5cnccc5o4)cc2)c2ccc(-c4ccc5c6c(cccc46)CC5)cc2)ccc13]

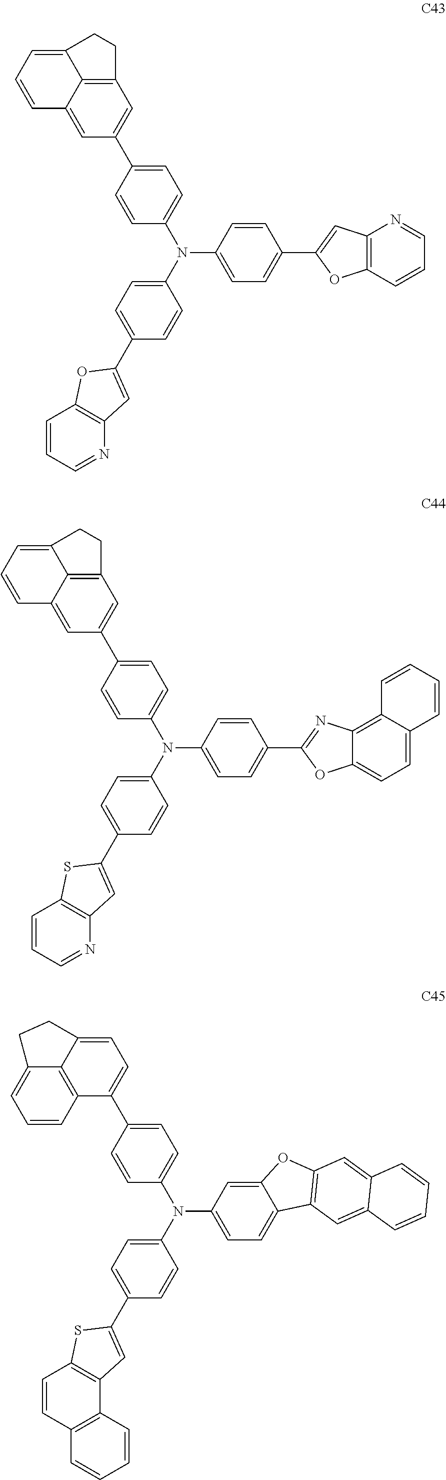 [SMILES: c1cc2c3c(cc(-c4ccc(N(c5ccc(-c6cc7ncccc7o6)cc5)c5ccc(-c6cc7ncccc7o6)cc5)cc4)cc3c1)CC2 ; c1cc2c3c(cc(-c4ccc(N(c5ccc(-c6nc7c(ccc8ccccc87)o6)cc5)c5ccc(-c6cc7ncccc7s6)cc5)cc4)cc3c1)CC2 ; c1ccc2cc3c(cc2c1)oc1cc(N(c2ccc(-c4cc5c(ccc6ccccc65)s4)cc2)c2ccc(-c4ccc5c6c(cccc46)CC5)cc2)ccc13]

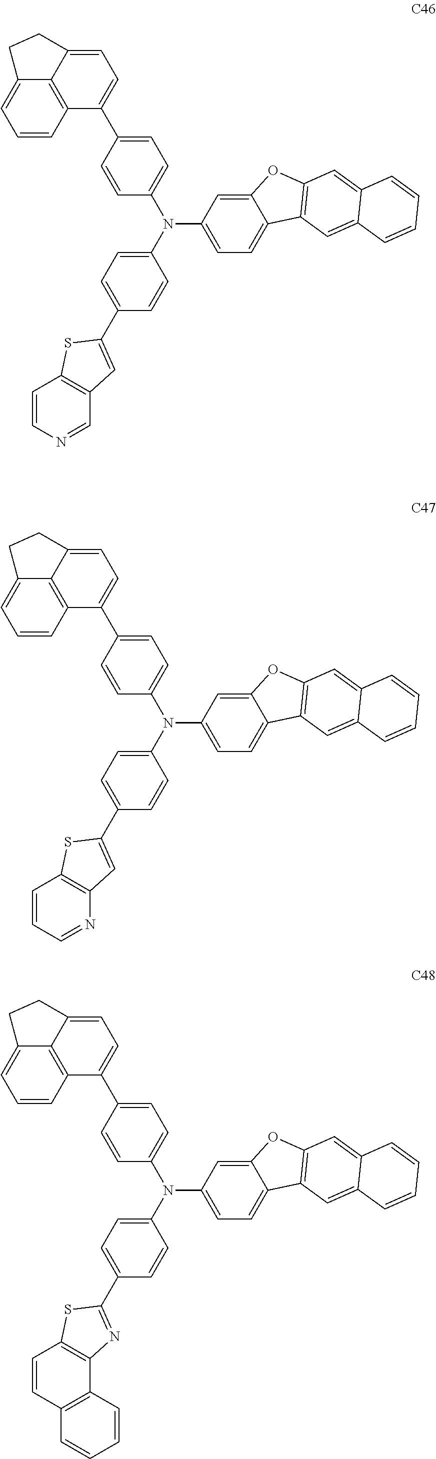 [SMILES: c1ccc2cc3c(cc2c1)oc1cc(N(c2ccc(-c4cc5cnccc5s4)cc2)c2ccc(-c4ccc5c6c(cccc46)CC5)cc2)ccc13 ; c1ccc2cc3c(cc2c1)oc1cc(N(c2ccc(-c4cc5ncccc5s4)cc2)c2ccc(-c4ccc5c6c(cccc46)CC5)cc2)ccc13 ; c1ccc2cc3c(cc2c1)oc1cc(N(c2ccc(-c4nc5c(ccc6ccccc65)s4)cc2)c2ccc(-c4ccc5c6c(cccc46)CC5)cc2)ccc13]

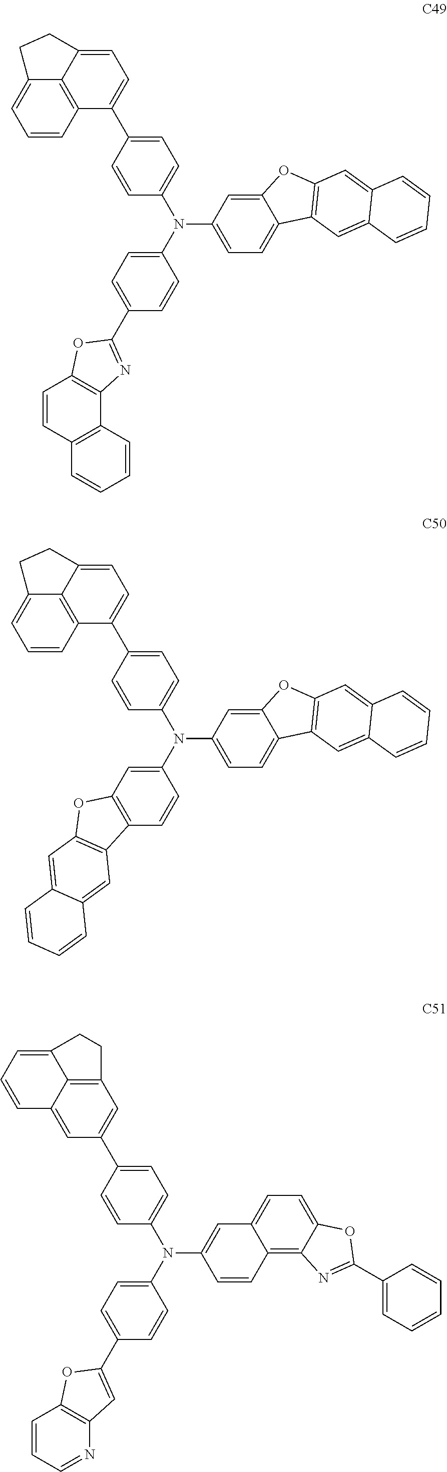 [SMILES: c1ccc2cc3c(cc2c1)oc1cc(N(c2ccc(-c4nc5c(ccc6ccccc65)o4)cc2)c2ccc(-c4ccc5c6c(cccc46)CC5)cc2)ccc13 ; c1ccc2cc3c(cc2c1)oc1cc(N(c2ccc(-c4ccc5c6c(cccc46)CC5)cc2)c2ccc4c(c2)oc2cc5ccccc5cc24)ccc13 ; c1ccc(-c2nc3c(ccc4cc(N(c5ccc(-c6cc7c8c(cccc8c6)CC7)cc5)c5ccc(-c6cc7ncccc7o6)cc5)ccc43)o2)cc1]

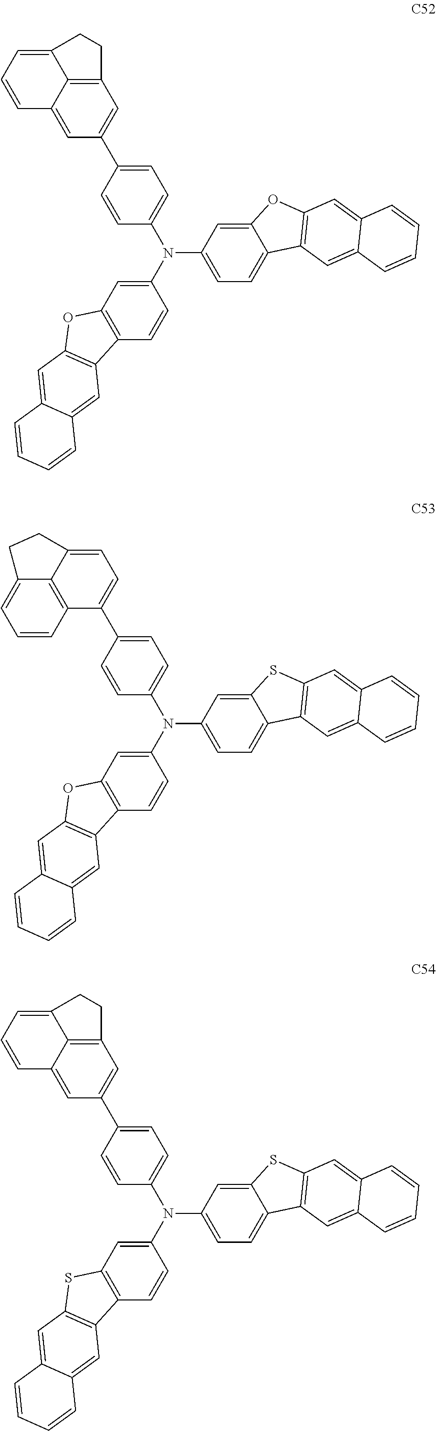 [SMILES: c1ccc2cc3c(cc2c1)oc1cc(N(c2ccc(-c4cc5c6c(cccc6c4)CC5)cc2)c2ccc4c(c2)oc2cc5ccccc5cc24)ccc13 ; c1ccc2cc3c(cc2c1)oc1cc(N(c2ccc(-c4ccc5c6c(cccc46)CC5)cc2)c2ccc4c(c2)sc2cc5ccccc5cc24)ccc13 ; c1ccc2cc3c(cc2c1)sc1cc(N(c2ccc(-c4cc5c6c(cccc6c4)CC5)cc2)c2ccc4c(c2)sc2cc5ccccc5cc24)ccc13]

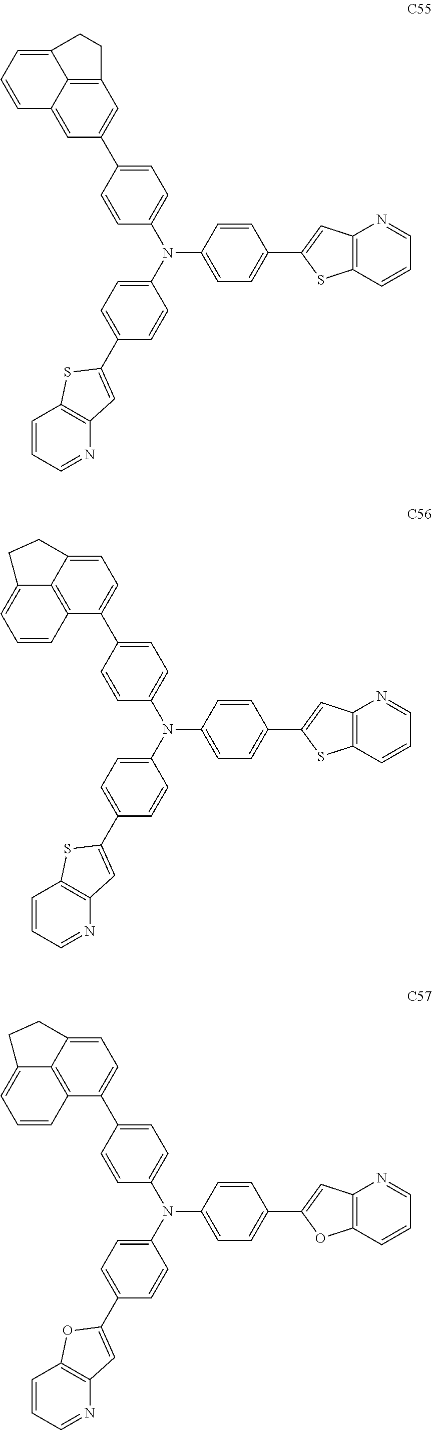 [SMILES: c1cc2c3c(cc(-c4ccc(N(c5ccc(-c6cc7ncccc7s6)cc5)c5ccc(-c6cc7ncccc7s6)cc5)cc4)cc3c1)CC2 ; c1cnc2cc(-c3ccc(N(c4ccc(-c5cc6ncccc6s5)cc4)c4ccc(-c5ccc6c7c(cccc57)CC6)cc4)cc3)sc2c1 ; c1cnc2cc(-c3ccc(N(c4ccc(-c5cc6ncccc6o5)cc4)c4ccc(-c5ccc6c7c(cccc57)CC6)cc4)cc3)oc2c1]

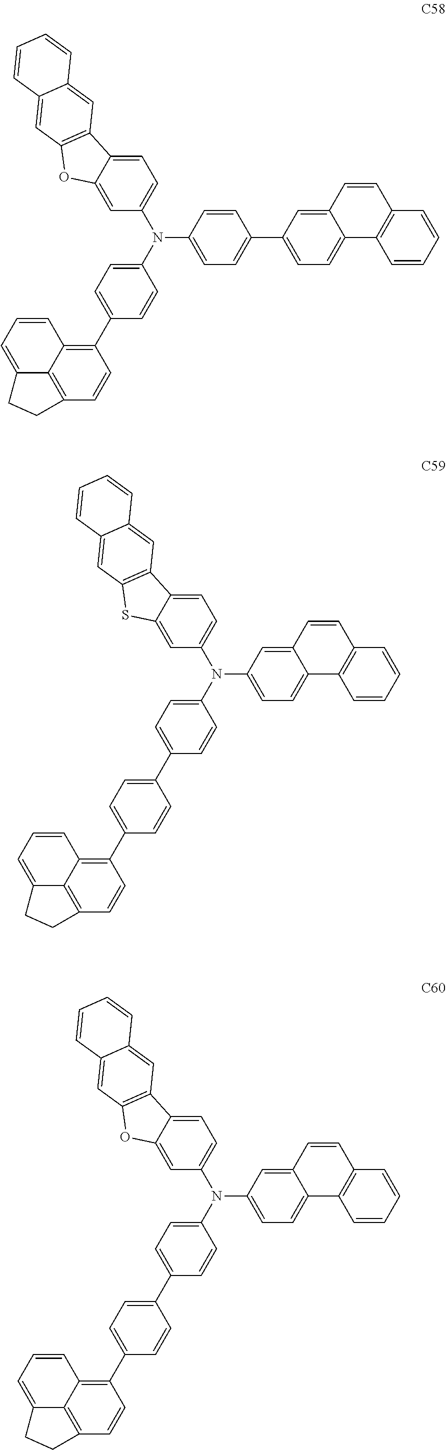 [SMILES: c1ccc2cc3c(cc2c1)oc1cc(N(c2ccc(-c4ccc5c(ccc6ccccc65)c4)cc2)c2ccc(-c4ccc5c6c(cccc46)CC5)cc2)ccc13 ; c1ccc2cc3c(cc2c1)sc1cc(N(c2ccc(-c4ccc(-c5ccc6c7c(cccc57)CC6)cc4)cc2)c2ccc4c(ccc5ccccc54)c2)ccc13 ; c1ccc2cc3c(cc2c1)oc1cc(N(c2ccc(-c4ccc(-c5ccc6c7c(cccc57)CC6)cc4)cc2)c2ccc4c(ccc5ccccc54)c2)ccc13]

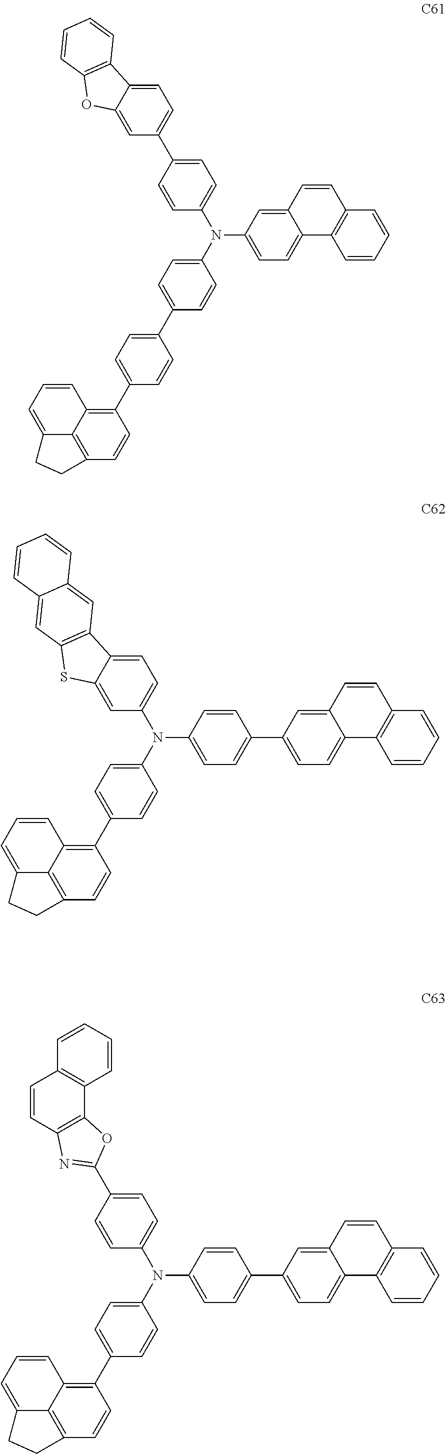 [SMILES: c1ccc2c(c1)ccc1cc(N(c3ccc(-c4ccc(-c5ccc6c7c(cccc57)CC6)cc4)cc3)c3ccc(-c4ccc5c(c4)oc4ccccc45)cc3)ccc12 ; c1ccc2cc3c(cc2c1)sc1cc(N(c2ccc(-c4ccc5c(ccc6ccccc65)c4)cc2)c2ccc(-c4ccc5c6c(cccc46)CC5)cc2)ccc13 ; c1ccc2c(c1)ccc1cc(-c3ccc(N(c4ccc(-c5nc6ccc7ccccc7c6o5)cc4)c4ccc(-c5ccc6c7c(cccc57)CC6)cc4)cc3)ccc12]

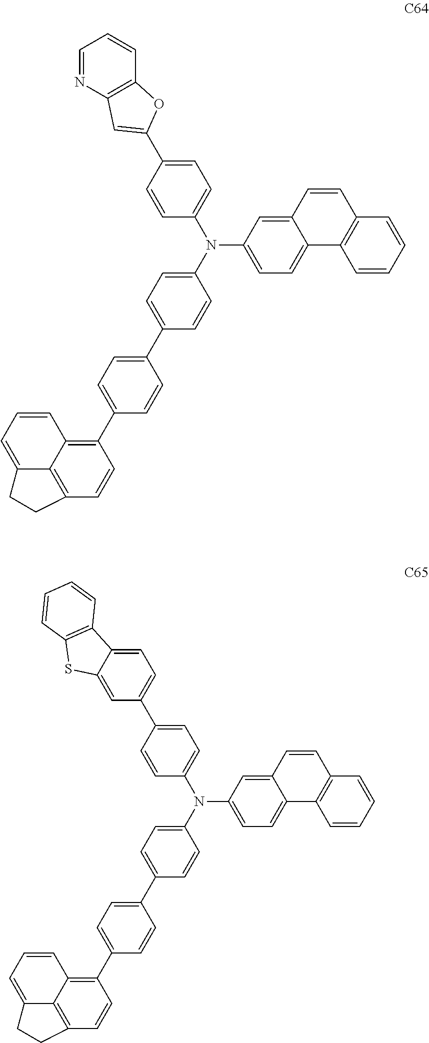 [SMILES: c1ccc2c(c1)ccc1cc(N(c3ccc(-c4ccc(-c5ccc6c7c(cccc57)CC6)cc4)cc3)c3ccc(-c4cc5ncccc5o4)cc3)ccc12 ; c1ccc2c(c1)ccc1cc(N(c3ccc(-c4ccc(-c5ccc6c7c(cccc57)CC6)cc4)cc3)c3ccc(-c4ccc5c(c4)sc4ccccc45)cc3)ccc12]

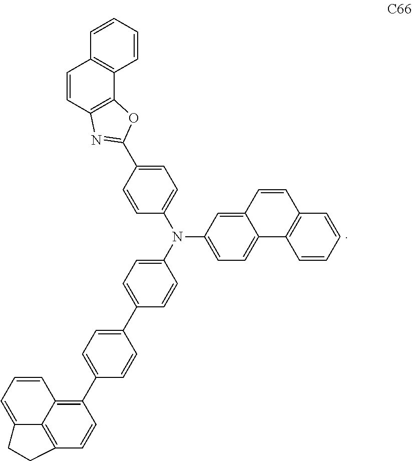 [SMILES: c1ccc2c(c1)ccc1cc(N(c3ccc(-c4ccc(-c5ccc6c7c(cccc57)CC6)cc4)cc3)c3ccc(-c4nc5ccc6ccccc6c5o4)cc3)ccc12]

In an embodiment of the present disclosure, the hole injection layer includes the aromatic amine compounds provided in the embodiments of the present disclosure, particularly including the first type or second type of aromatic amine compounds. For example, the material for the hole injection layer may be a mixture of the first type of aromatic amine compounds provided in the embodiments of the present disclosure with a doping material, or a mixture of the second type of aromatic amine compound provided in the embodiments of the present disclosure with a doping material.

In other embodiments of the present disclosure, the hole injection layer may employ materials other than the aromatic amine compounds provided herein, such as inorganic oxides including but not limited to molybdenum oxide, silver oxide, tungsten oxide, manganese oxide, and other inorganic oxides. Alternatively, it may be selected from p-type dopants with strong electron-withdrawing systems and doped hole-transport materials, such as F4TCNQ, HATCN, and the like.

In an embodiment of the present disclosure, the hole transport layer may include the aromatic amine compounds provided in the embodiments of the present disclosure, particularly including the first type or second type of aromatic amine compounds. In an example, the material for the hole transport layer may be the first type of aromatic amine compound provided in the embodiments of the present disclosure, or it may be the second type of aromatic amine compound provided in the embodiments of the present disclosure. In another example, the hole transport layer may comprise multiple sequentially stacked sub-hole transport layers, such as including a first sub-hole transport layer and a second sub-hole transport layer stacked in sequence. The material of either the first sub-hole transport layer or the second sub-hole transport layer may be the first type or second type of aromatic amine compound provided in the embodiments of the present disclosure. In yet another example, the hole transport layer may include multiple sequentially stacked sub-hole transport layers, for example including a first sub-hole transport layer and a second sub-hole transport layer stacked in sequence. The materials of both the first sub-hole transport layer and the second sub-hole transport layer are selected from the first type or second type of aromatic amine compounds provided in the embodiments of this disclosure, with the materials of the first and second sub-hole transport layers being different from each other.

In other embodiments of the present disclosure, the hole transport layer may employ materials other than the aromatic amine compounds provided herein, such as aromatic amine-based hole-transport materials or carbazole-based hole-transport materials, including but not limited to one or more of NPB, TPD, BAFLP, 4DFLDPBi, CBP, PCzPA, or TCTA.

In an example, the hole injection layer and hole transport layer may be formed sequentially via evaporation using an open metal mask. The thickness of the hole injection layer is 5 to 20 nm. The thickness of the hole transport layer is 80 to 120 nm.

In an embodiment of the present disclosure, the electron barrier layer (also referred to as a light-emitting auxiliary layer in some contexts) includes the aromatic amine compounds provided in the embodiments of the present disclosure, particularly including the first type or second type of aromatic amine compounds. For example, the material for the electron barrier layer may be the first type of aromatic amine compound or the second type of aromatic amine compound provided in the embodiments of the present disclosure.

In other embodiments of the present disclosure, the electron barrier layer may employ materials other than the aromatic amine compounds provided herein, including but not limited to one or more of NPB, TPD, BAFLP, 4DFLDPBi, CBP, PCzPA, or TCTA.

In an example of the present disclosure, the hole injection layer includes the same material as that of the hole transport layer. In other words, the hole injection layer includes two or more materials, one of which is the same as the material in the hole transport layer.

In an embodiment of the present disclosure, the photoluminescence (PL) wavelength peak of the first functional layer does not exceed 450 nm, for example, it lies within the range of 400 nm to 450 nm. This ensures that only a minimal portion or none of the light emitted by the light-emitting functional layer possesses sufficient energy to induce luminescence in the first functional layer, thereby preventing degradation in the color purity of the light output from the electroluminescent device.

In an embodiment of the present disclosure, The PL wavelength peak of the first functional layer is λ1; the PL wavelength peak of the light-emitting functional layer is λ2; the difference λ2-λ1 is not less than 5 nm, particularly not less than 20 nm. This configuration ensures that the photoluminescence from the light-emitting functional layer does not induce photoluminescence in the first functional layer.

Optionally, to measure the PL spectrum of each material, the organic material is vapor-deposited to form a single-layer thin film with a thickness of 50 nm, and then the single-layer thin film is subjected to photoluminescence testing using a fluorescence spectrometer.

In an embodiment of the present disclosure, the HOMO (highest occupied molecular orbital) energy level of the electron barrier layer is equal to or lower than that of the hole transport layer. Further, the absolute difference between the HOMO energy level of the hole transport layer and that of the electron barrier layer is not less than 0.05 eV and not greater than 0.25 eV.

In an embodiment of the present disclosure, the thickness of the hole transport layer is greater than that of the electron barrier layer.

In an embodiment of the present disclosure, a display panel may include sub-pixels arranged in an array, each sub-pixel being an electroluminescent device (for example, all are OLEDs). In the example of FIG. 2, the display device includes a red electroluminescent device, a green electroluminescent device, and a blue electroluminescent device. In the example of FIG. 2, any electroluminescent device includes an anode, a hole injection layer (HIL), a hole transport layer (HTL), a light-emitting auxiliary layer (Prime, for example, a red light-emitting auxiliary layer R-Prime for the red electroluminescent device, a green light-emitting auxiliary layer G-Prime for the green electroluminescent device, a blue light-emitting auxiliary layer B-Prime for the blue electroluminescent device), an organic light-emitting functional layer (EML, for example, a red organic light-emitting functional layer R-EML, a green organic light-emitting functional layer G-EML, a blue organic light-emitting functional layer B-EML), a hole barrier layer (HBL), an electron transport layer (ETL), an electron injection layer (EIL), and a cathode, which are sequentially stacked.

In an example, THK(R-Prime)>THK(G-Prime)>THK(B-Prime).

THK(R-Prime) is the thickness of the light-emitting auxiliary layer R-Prime of the red electroluminescent device; THK(G-Prime) is the thickness of the light-emitting auxiliary layer G-Prime of the green electroluminescent device; THK(B-Prime) is the thickness of the light-emitting auxiliary layer B-Prime of the blue electroluminescent device.

In an example, THK(R-Prime)>THK(G-Prime)+THK(B-Prime).

In an example, THK(HTL)>THK(G-Prime)+THK(B-Prime). Wherein, THK(HTL) is the thickness of the hole transport layer (HTL).

In an example, the thickness of the hole transport region film layer (i.e., the thickness of the first functional layer)>the thickness of the electron transport region (i.e., the thickness of the second functional layer).

In an example, THK(HTL)>THK(HBL)+THK(ETL)+THK(EIL). Wherein, THK(HBL) is the thickness of the hole barrier layer; THK(ETL) is the thickness of the electron transport layer; THK(EIL) is the thickness of the electron injection layer.

In an example, THK(HTL)≥100 nm, 50 nm≤THK(R-Prime)<100 nm, 1 nm<THK(B-Prime)<30 nm; 25 nm<THK(G-Prime)≤50 nm.

In the above examples, the microcavities of the red, green, and blue electroluminescent devices can be adjusted to achieve optimal white light efficiency in the display panel. Microcavity adjustment is primarily achieved by regulating the thickness of the hole transport region. Due to the poor blue light efficiency and high driving voltage, which are related to the energy level barriers and hole mobility between the hole transport layer, light-emitting auxiliary layer, and light-emitting functional layer, in an example, the performance of blue light can be improved by enhancing the energy level and mobility of the light-emitting auxiliary layer in the blue electroluminescent device. The optimal white light efficiency is achieved through microcavity structures with varying thicknesses.

In an embodiment of the present disclosure, the light-emitting functional layer is an organic light-emitting functional layer, which may include a host material and a guest material. In an example, the host material may be a single material, such as a host material simultaneously possessing electron transport and hole transport functionalities. In another example, the host material may include a hole-type host material primarily for hole transport and an electron-type host material primarily for electron transport. The guest material may be a fluorescent material or a phosphorescent material. Of course, depending on requirements, the organic light-emitting functional layer may also adopt other implementations.

In an example, the organic light-emitting functional layer may be a blue organic light-emitting functional layer; holes and electrons recombine within the blue organic light-emitting functional layer, causing it to emit blue light. Optionally, the host material of the blue organic light-emitting functional layer includes but is not limited to anthracene derivative; the anthracene derivative blue light materials are selected from AND and/or MAND; the guest material of the blue organic light-emitting functional layer includes but is not limited to pyrene derivative and/or the styrene derivative DPVBi.

In another example, the host material may be a carbazole derivative or an azine derivative; the guest material may be an organometallic complexe (e.g., an organoiridium complexe or organoplatinum complexe), and the like.

In an embodiment of the present disclosure, a fine metal mask (FMM) may be used to vapor-deposit and form an electron barrier layer (in some embodiments, also referred to as the light-emitting auxiliary layer) and the organic light-emitting functional layer. In this embodiment, the electroluminescent device is an organic electroluminescent device (OLED).

In an example, the thickness of the light-emitting auxiliary layer in the blue electroluminescent device is between 5 to 20 nm; the light-emitting functional layer (organic light-emitting functional layer) of the blue electroluminescent device includes a Blue light host material and a Blue light guest material, with a thickness of 10 to 30 nm; the doping ratio of the Blue light guest material is 1% to 10%. For example, the doping ratio of the Blue light guest material is 4%.

In an example, the thickness of the light-emitting auxiliary layer in the green electroluminescent device is 5 to 20 nm; the light-emitting functional layer (organic light-emitting functional layer) of the green electroluminescent device includes a green host material and a green guest material, with a thickness of 20 to 50 nm; the doping ratio of the green guest material is 1% to 15%. For example, the doping ratio of the green guest material is 10%.

In an example, the thickness of the light-emitting auxiliary layer in the red electroluminescent device is 50 to 100 nm; the light-emitting functional layer (organic light-emitting functional layer) of the red electroluminescent device includes a red host material and a red guest material, with a thickness of 50 to 100 nm; the doping ratio of the red guest material is 1% to 15%. For example, the doping ratio of the red guest material is 2%.

In the example of FIG. 2, the display device includes a red electroluminescent device, a green electroluminescent device, and a blue electroluminescent device. It should be understood that in other embodiments of the present disclosure, the colors of the electroluminescent devices included in the display device may differ from the example in FIG. 2. For instance, at least one of the red, green, or blue electroluminescent devices may be omitted, or electroluminescent devices of new colors may be added.

In an embodiment of the present disclosure, the hole barrier layer may adopt aromatic heterocyclic compounds, including but not limited to one or more of imidazole derivatives, azine derivatives, quinoline derivatives, isoquinoline derivatives, or phenanthroline derivatives. In an example, the material of the hole barrier layer is a compound with a nitrogen-containing six-membered ring structure or a compound containing phosphine oxide groups on the heterocyclic ring. The imidazole derivatives include but are not limited to one or more of benzimidazole derivatives, imidazopyridine derivatives, or benzimidazophenanthridine derivatives; the azine derivatives include pyrimidine derivatives and/or triazine derivatives. Furthermore, the materials of the electron transport layer and the hole barrier layer include but are not limited to one or more of PBD, OXD-7, TAZ, p-EtTAZ, BCP, and TPBI.

In an embodiment of the present disclosure, the electron transport layer may adopt, but is not limited to, one or more of imidazole derivatives, azine derivatives, quinoline derivatives, isoquinoline derivatives, or phenanthroline derivatives. In an example, the material of the hole barrier layer is a compound with a nitrogen-containing six-membered ring structure or a compound containing phosphine oxide groups on the heterocyclic ring. The imidazole derivatives include but are not limited to one or more of benzimidazole derivatives, imidazopyridine derivatives, or benzimidazophenanthridine derivatives; the azine derivatives include pyrimidine derivatives and/or triazine derivatives. Furthermore, the materials of the electron transport layer and the hole barrier layer include but are not limited to one or more of PBD, OXD-7, TAZ, p-EtTAZ, BCP, and TPBI.

In other embodiments of the present disclosure, the electron transport layer may also adopt inorganic materials, such as doped or undoped zinc oxide.

In an embodiment of the present disclosure, the electron injection layer may be an alkali metal or metal, such as LiF, Yb, Mg, or Ca.

In an embodiment of the present disclosure, the materials of at least two layers among the electron injection layer, electron transport layer, and hole barrier layer contain triazine groups, which can provide the second functional layer with excellent electron transport efficiency.

In an example, the photoluminescence wavelength (peak) range of the second functional layer is 390 to 430 nm.

In an embodiment of the present disclosure, the peak photoluminescence wavelength of the second functional layer is λ3, and the peak photoluminescence wavelength of the light-emitting functional layer is λ2, where λ2-λ3 is greater than 20 nm, especially not less than 30 nm. This ensures that the photoluminescence of the light-emitting functional layer does not induce photoluminescence in the second functional layer.

In an embodiment of the present disclosure, an open mask may be used to vapor-deposit and form the hole barrier layer, electron transport layer, and electron injection layer separately; the thickness of the hole barrier layer is 1 to 20 nm; the thickness of the electron transport layer is 10 to 50 nm; and the thickness of the electron injection layer is 1 to 10 nm.

In an embodiment of the present disclosure, the anode is a high work function electrode material, which can inject holes into the first functional layer.

In an example, the anode may be a transparent conductive metal oxide, such as ITO or IZO. Further, the thickness of the anode is between 80 to 200 nm. Optionally, the anode is transparent, and the electroluminescent device emits light through the anode side. Further, the anode is adjacent to the driving layer, and the electroluminescent device is a bottom-emission device.

In another example, the anode may include a metal layer as a reflective layer. For instance, the anode is a composite electrode formed by Ag/ITO, AU/ITO, Ag/IZO, or AU/IZO, where the metal thickness in the composite electrode is 10 to 100 nm, and the oxide layer thickness is 5 to 20 nm. The oxide layer is in direct contact with the first functional layer. In this example, the electroluminescent device cannot emit light through the anode and thus emits light through the cathode side. Further, the anode is adjacent to the driving layer, and the electroluminescent device is a top-emission device.

In an embodiment of the present disclosure, the cathode may use low work function metals (e.g., Al, Ag, Mg) or alloys containing low work function metal materials (e.g., Al—Mg alloy). Of course, the cathode may also adopt conductive metal oxides such as IGZO.

Optionally, when the electroluminescent device emits light through the anode side, the cathode may have a larger thickness to achieve higher reflectivity, for example, a thickness of not less than 80 nm. Further, the cathode has a reflectance greater than 85% at 550 nm.

Optionally, when the electroluminescent device emits light through the cathode side, the cathode may have a smaller thickness to achieve higher transmittance, for example, a thickness in the range of 10 to 20 nm. Further, the cathode has a transmittance greater than 45% at 550 nm.

In an embodiment of the present disclosure, an open mask may be used to sequentially vapor-deposit and form the metal cathode and a cover layer.

In an embodiment of the present disclosure, the material of the cover layer has a high refractive index to improve the light extraction efficiency of the electroluminescent device. For example, the material of the cover layer has a refractive index of not less than 1.8 at 460 nm.

Further, the material of the cover layer exhibits high transmittance for light with wavelengths greater than 450 nm, for instance, its absorption coefficient k is less than 0.1.

In related technologies, materials for the cover layer also show low absorption coefficients for shorter-wavelength light, such as maintaining a low absorption coefficient for light around 400 nm. On one hand, such cover layers cannot filter or absorb short-wavelength light; on the other hand, environmental short-wavelength light may penetrate the cover layer and irradiate the functional layers, potentially accelerating their aging.

In an embodiment of the present disclosure, the cover layer may adopt the third aromatic amine compound provided in this disclosure. This aromatic amine compound not only has a high refractive index but also exhibits low absorbance for light with wavelengths greater than 450 nm (e.g., light in the 450 to 630 nm range, facilitating visible light emission) and strong absorption for light at 400 nm or shorter wavelengths. Thus, the cover layer not only enhances the light extraction efficiency of the electroluminescent device through its high refractive index without compromising normal visible light emission but also protects the functional layers by absorbing short-wavelength light (especially ultraviolet light), thereby improving device longevity.

Below, exemplary compounds are synthesized and their performance is tested.

### Synthesis of Intermediate Compound C-1

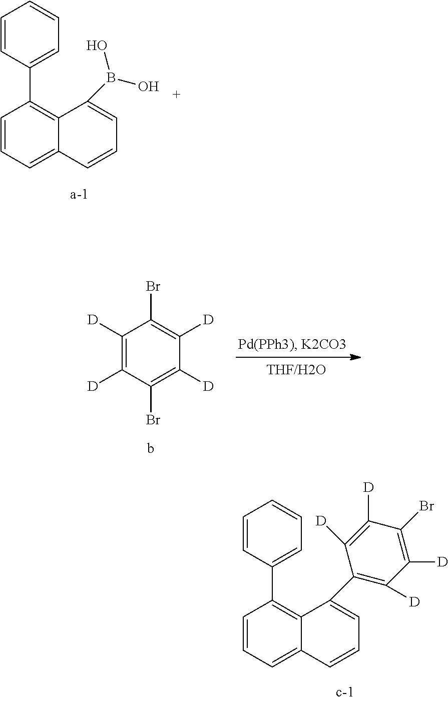 [SMILES: OB(O)c1cccc2cccc(-c3ccccc3)c12 ; [2H]c1c([2H])c(Br)c([2H])c([2H])c1Br ; [2H]c1c([2H])c(-c2cccc3cccc(-c4ccccc4)c23)c([2H])c([2H])c1Br]

Compound a-1 [(8-phenyl-1-naphthyl)boronic acid](24.81 g, 100 mmol) and Compound b [1,4-deuterated p-bromobiphenyl](23.99 g, 100 mmol) were added to a reaction flask, followed by 250 mL of a THF/water mixed solvent (volume ratio 1-5:1). After heating under reflux for 1 h under a nitrogen atmosphere, the temperature was reduced to below 80° C. Potassium carbonate (27.64 g, 200 mmol) was added, followed by [tetrakis(triphenylphosphine)palladium(0)](1.16 g, 1 mmol). The mixture was heated to reflux and reacted for 8-12 hours. Upon completion of the reaction of the reaction, the reaction mixture was washed with water, and the organic phase was extracted with dichloromethane. The organic phase was dried over anhydrous magnesium sulfate, filtered, and concentrated by rotary evaporation to obtain a crude product. The crude product was recrystallized using dichloromethane and n-heptane to yield Intermediate Compound c-1 (27.25 g, 75% yield). LC/MS (m/z) (M+): 362.05.

### Synthesis of Intermediate Compound C-2

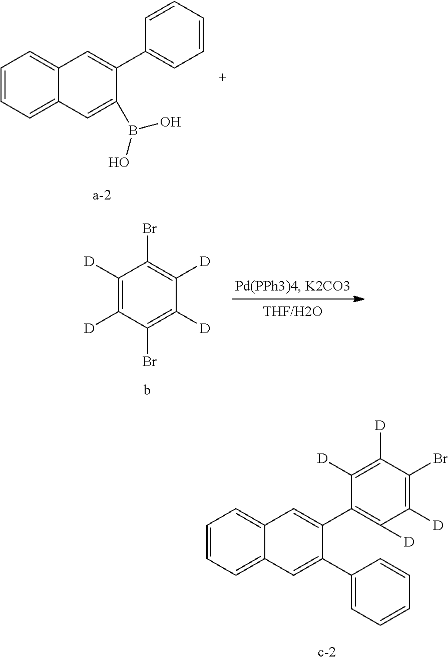 [SMILES: OB(O)c1cc2ccccc2cc1-c1ccccc1 ; [2H]c1c([2H])c(Br)c([2H])c([2H])c1Br ; [2H]c1c([2H])c(-c2cc3ccccc3cc2-c2ccccc2)c([2H])c([2H])c1Br]

Compound a-2 [(3-phenyl-2-naphthyl)boronic acid](24.81 g, 100 mmol) and Compound b [1,4-deuterated p-bromobiphenyl](23.99 g, 100 mmol) were added to a reaction flask, followed by 250 mL of a THF/water mixed solvent (volume ratio 1-5:1). After heating under reflux for 1 h under a nitrogen atmosphere, the temperature was reduced to below 80° C. Potassium carbonate (27.64 g, 200 mmol) was added, followed by [tetrakis(triphenylphosphine)palladium(0)](1.16 g, 1 mmol). The mixture was heated to reflux and reacted for 8-12 hours. Upon completion of the reaction of the reaction, the reaction mixture was washed with water, and the organic phase was extracted with dichloromethane. The organic phase was dried over anhydrous magnesium sulfate, filtered, and concentrated by rotary evaporation to obtain a crude product. The crude product was recrystallized using dichloromethane and n-heptane to yield Intermediate Compound c-2 (24.62 g, 68% yield). LC/MS (m/z)(M+): 362.06

Other Intermediate compounds can be prepared by adjusting the appropriate reactants following the above steps.

### Synthesis of Compound 5

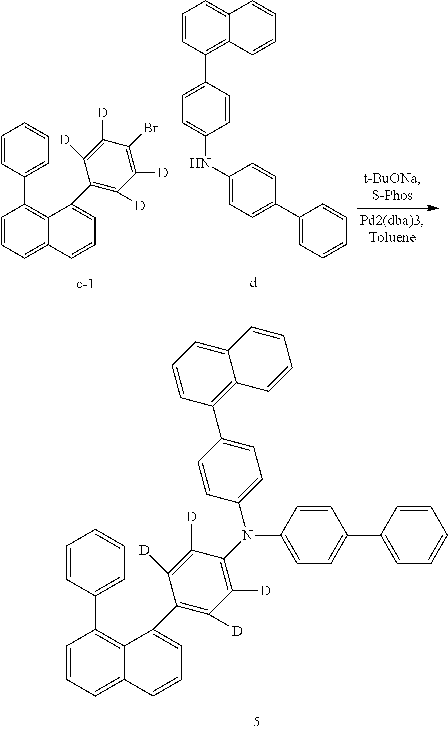

Intermediate Compound c-1 (3.63 g, 10 mmol) and compound d [N-(4-(1-naphthyl)phenyl)-4-biphenylamine](3.71 g, 10 mmol) were added to a reaction flask, followed by the addition of 100 ml toluene. Under a nitrogen atmosphere, the mixture was heated to reflux for 2 hours and then cooled. Sodium tert-butoxide (1.44 g, 15 mmol), 2-dicyclohexylphosphino-2′,6′-dimethoxybiphenyl (0.082 g, 0.2 mmol), and tris(dibenzylideneacetone)dipalladium(0) (0.092 g, 10 mmol) were added, and the mixture was heated to reflux and reacted for 8-12 hours. Upon completion of the reaction, the reaction was quenched with water, and dichloromethane and water were added for extraction. The organic phase was dried with anhydrous magnesium sulfate, concentrated by rotary evaporation, and purified by silica gel column chromatography. Recrystallization with toluene and n-heptane yielded obtain compound 5 (5.35 g, 82%). The purity was determined by HPLC to be 99%. LC/MS (m/z) (M+): 653.31.

### Synthesis of Compound 23

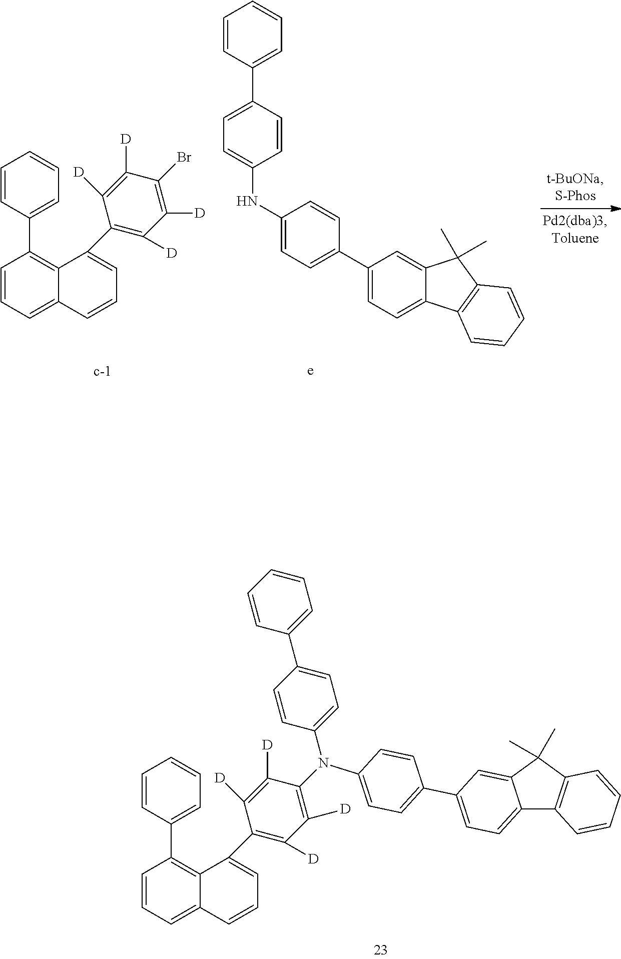

Intermediate Compound c-1 (3.63 g, 10 mmol) and Compound e [N-(4-(9,9-dimethyl-9H-fluoren-2-yl)phenyl)-9,9-dimethyl-9H-fluoren-2-amine](4.77 g, 10 mmol) were added to a reaction flask, followed by the addition of 100 ml toluene. Under a nitrogen atmosphere, the mixture was heated to reflux for 2 hours, and then cooled. Sodium tert-butoxide (1.44 g, 15 mmol), 2-dicyclohexylphosphino-2′,6′-dimethoxybiphenyl (0.082 g, 0.2 mmol), and tris(dibenzylideneacetone)dipalladium(0) (0.092 g, 0.1 mmol) were added, and the mixture was heated to reflux for 8-12 hours. Upon completion of the reaction of the reaction, water was added to quench the reaction, followed by extraction with dichloromethane and water. The organic phase was dried over anhydrous magnesium sulfate, concentrated by rotary evaporation, and purified by silica gel column chromatography. Recrystallization with toluene and n-heptane yielded compound 23 (5.61 g, 78% yield). The purity was determined by HPLC to be 99.10%. LC/MS (m/z) (M+): 719.34.

### Synthesis of Compound 72

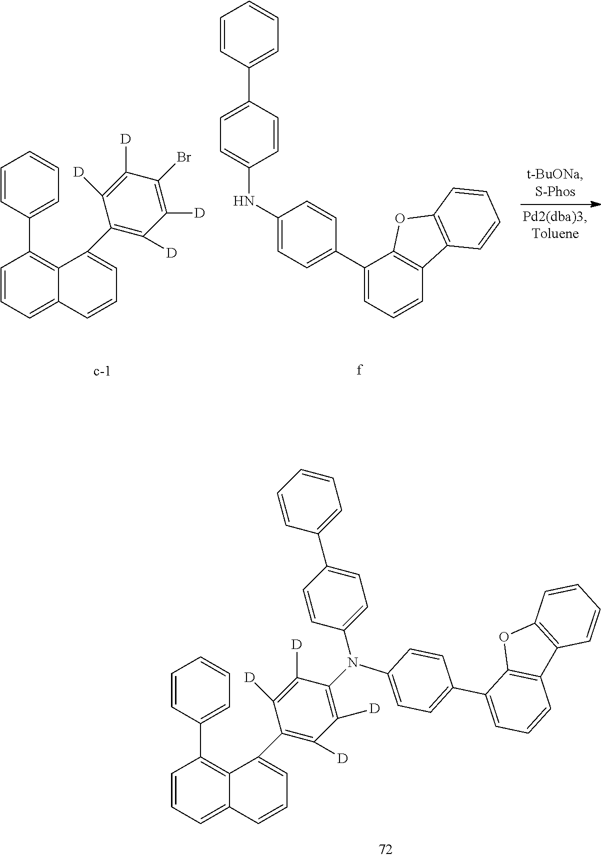

Intermediate Compound c-1 (3.63 g, 10 mmol) and Compound f [N-[4-(3-dibenzofuranyl)phenyl]-[1,1′-biphenyl]-4-amine](4.12 g, 10 mmol) were added to a reaction flask, followed by the addition of 100 ml toluene. Under a nitrogen atmosphere, the mixture was heated to reflux for 2 hours, and then cooled. Sodium tert-butoxide (1.44 g, 15 mmol), 2-dicyclohexylphosphino-2′,6′-dimethoxybiphenyl (0.082 g, 0.2 mmol), and tris(dibenzylideneacetone)dipalladium(0) (0.092 g, 0.1 mmol) were added, and the mixture was heated to reflux for 8-12 hours. Upon completion of the reaction of the reaction, water was added to quench the reaction, followed by extraction with dichloromethane and water. The organic phase was dried over anhydrous magnesium sulfate, concentrated by rotary evaporation, and purified by silica gel column chromatography. Recrystallization with toluene and n-heptane yielded compound 72 (4.78 g, 69% yield). The purity was determined by HPLC to be 99.35%. LC/MS (m/z)(M+): 693.29.

### Synthesis of Compound 85

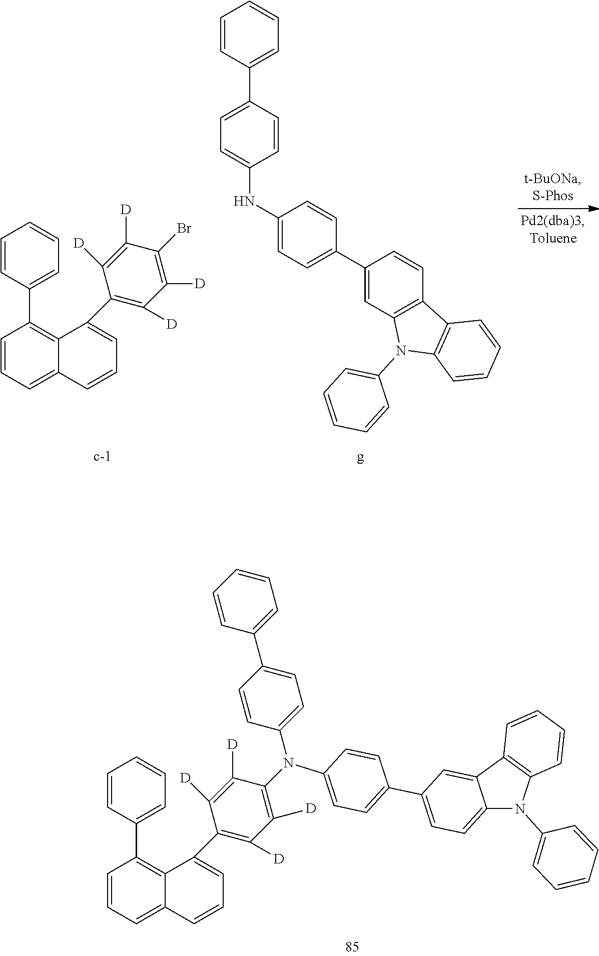 [SMILES: [2H]c1c([2H])c(-c2cccc3cccc(-c4ccccc4)c23)c([2H])c([2H])c1Br ; c1ccc(-c2ccc(Nc3ccc(-c4ccc5c6ccccc6n(-c6ccccc6)c5c4)cc3)cc2)cc1 ; [2H]c1c([2H])c(N(c2ccc(-c3ccccc3)cc2)c2ccc(-c3ccc4c(c3)c3ccccc3n4-c3ccccc3)cc2)c([2H])c([2H])c1-c1cccc2cccc(-c3ccccc3)c12]

Intermediate Compound c-1 (3.63 g, 10 mmol) and Compound g [N-(4-(9-phenyl-9H-carbazol-3-yl)phenyl)-[1,1′-biphenyl]-4-amine](4.86 g, 10 mmol) were added to a reaction flask, followed by 100 mL of toluene. The mixture was heated to reflux for 2 hours under a nitrogen atmosphere and then cooled. Sodium tert-butoxide (1.44 g, 15 mmol), 2-dicyclohexylphosphino-2′,6′-dimethoxybiphenyl (0.082 g, 0.2 mmol), and bis(dibenzylideneacetone)palladium(0) (0.092 g, 0.1 mmol) were added, and the mixture was heated to reflux for 8-12 hours. Upon completion of the reaction, the reaction was quenched with water and extracted with dichloromethane and water. The organic phase was dried over anhydrous magnesium sulfate, concentrated by rotary evaporation, and purified by silica gel column chromatography. Recrystallization with toluene and n-heptane yielded compound 85 (5.84 g, 76% yield). The purity was determined by HPLC to be 99%. LC/MS (m/z)(M+): 768.34.

### Synthesis of Compound 170

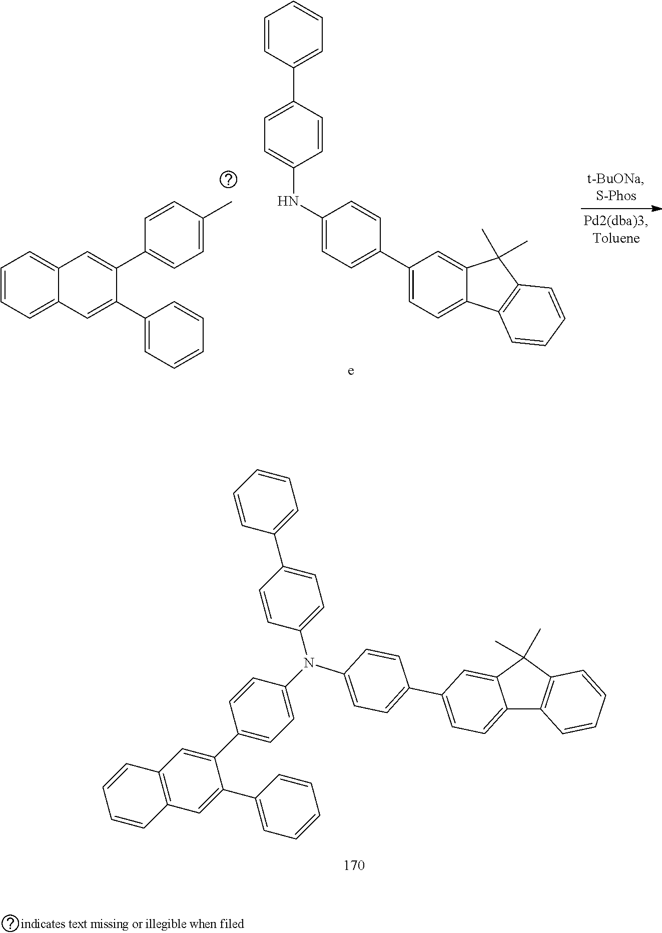

2-(4-Bromophenyl)-3-phenylnaphthalene (3.63 g, 10 mmol) and Compound e [N-(4-(9,9-dimethyl-9H-fluoren-2-yl)phenyl)-9,9-dimethyl-9H-fluoren-2-amine](4.77 g, 10 mmol) were added to a reaction flask, followed by 100 mL of toluene. The mixture was heated to reflux for 2 hours under a nitrogen atmosphere, and then cooled. Sodium tert-butoxide (1.44 g, 15 mmol), 2-dicyclohexylphosphino-2′,6′-dimethoxybiphenyl (0.082 g, 0.2 mmol), and bis(dibenzylideneacetone)palladium(0) (0.092 g, 0.1 mmol) were added, and the mixture was heated to reflux for 8-12 hours. Upon completion of the reaction, the reaction was quenched with water and extracted with dichloromethane and water. The organic phase was dried over anhydrous magnesium sulfate, concentrated by rotary evaporation, and purified by silica gel column chromatography. Recrystallization with toluene and n-heptane yielded compound 170 (5.79 g, 81% yield). The purity was determined by HPLC to be 99.11%. LC/MS (m/z)(M+): 715.32.

### Synthesis of Compound 187

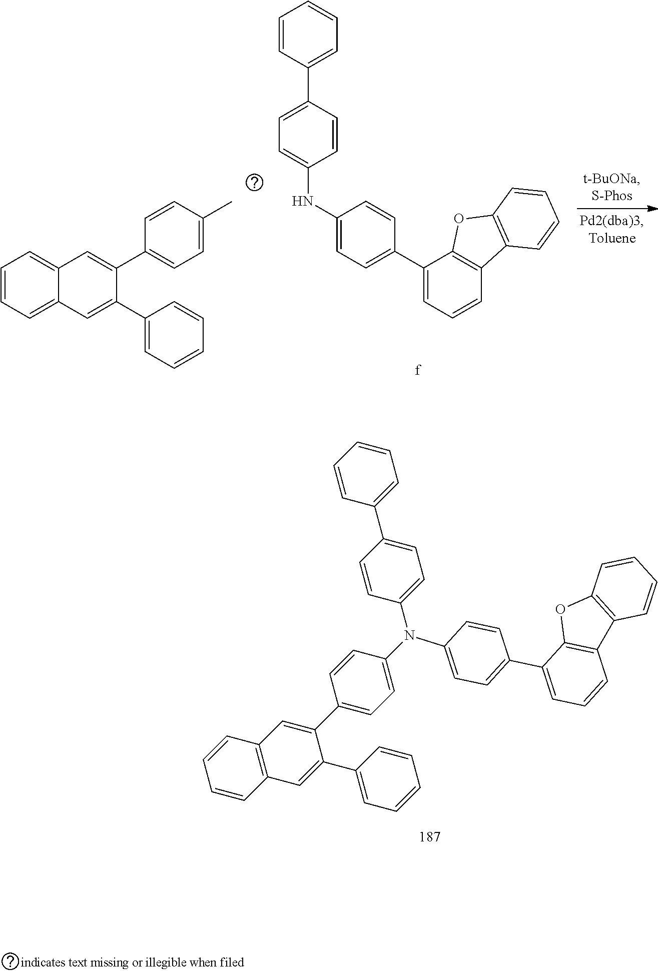

2-(4-Bromophenyl)-3-phenylnaphthalene (3.63 g, 10 mmol) and Compound f [N-[4-(dibenzofuran-3-yl)phenyl]-[1,1′-biphenyl]-4-amine](4.12 g, 10 mmol) were added to a reaction flask, followed by 100 mL of toluene. The mixture was heated to reflux for 2 hours under a nitrogen atmosphere, and then cooled. Sodium tert-butoxide (1.44 g, 15 mmol), 2-dicyclohexylphosphino-2′,6′-dimethoxybiphenyl (0.082 g, 0.2 mmol), and bis(dibenzylideneacetone)palladium(0) (0.092 g, 0.1 mmol) were added, and the mixture was heated to reflux for 8-12 hours. Upon completion of the reaction, the reaction was quenched with water and extracted with dichloromethane and water. The organic phase was dried over anhydrous magnesium sulfate, concentrated by rotary evaporation, and purified by silica gel column chromatography. Recrystallization with toluene and n-heptane yielded compound 187 (4.75 g, 69% yield). The purity was determined by HPLC to be 99.24%. LC/MS (m/z)(M+): 689.27.

### Synthesis of Compound 196

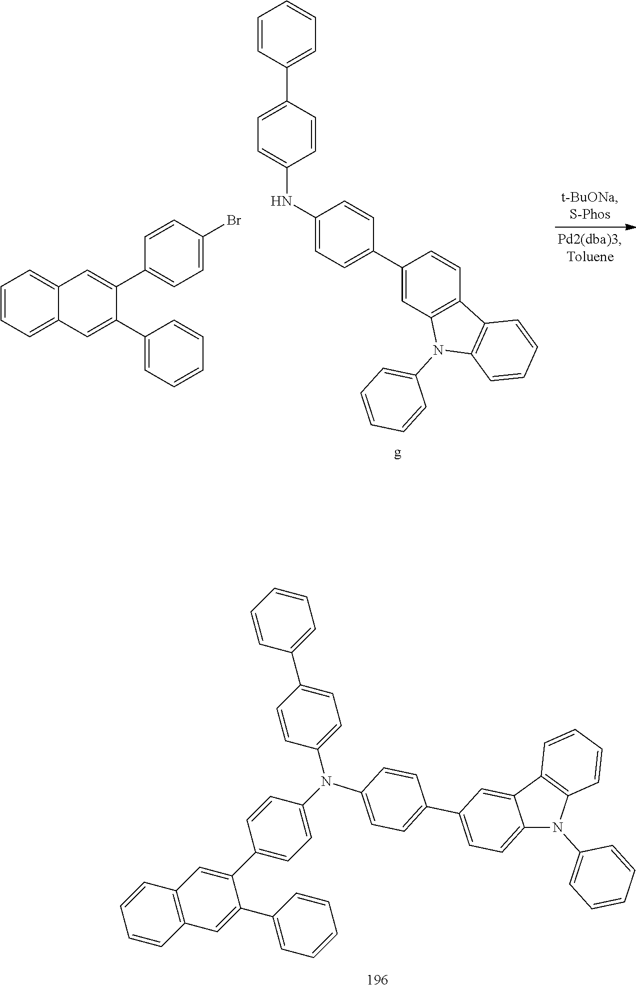 [SMILES: Brc1ccc(-c2cc3ccccc3cc2-c2ccccc2)cc1 ; c1ccc(-c2ccc(Nc3ccc(-c4ccc5c6ccccc6n(-c6ccccc6)c5c4)cc3)cc2)cc1 ; c1ccc(-c2ccc(N(c3ccc(-c4ccc5c(c4)c4ccccc4n5-c4ccccc4)cc3)c3ccc(-c4cc5ccccc5cc4-c4ccccc4)cc3)cc2)cc1]

2-(4-Bromophenyl)-3-phenylnaphthalene (3.63 g, 10 mmol) and Compound g [N-(4-(9-phenyl-9H-carbazol-3-yl)phenyl)-[1,1′-biphenyl]-4-amine](4.86 g, 10 mmol) were added to a reaction flask, followed by 100 mL of toluene. The mixture was heated to reflux for 2 hours under a nitrogen atmosphere, and then cooled. Sodium tert-butoxide (1.44 g, 15 mmol), 2-dicyclohexylphosphino-2′,6′-dimethoxybiphenyl (0.082 g, 0.2 mmol), and bis(dibenzylideneacetone)palladium(0) (0.092 g, 0.1 mmol) were added, and the mixture was heated to reflux for 8-12 hours. Upon completion of the reaction, the reaction was quenched with water and extracted with dichloromethane and water. The organic phase was dried over anhydrous magnesium sulfate, concentrated by rotary evaporation, and purified by silica gel column chromatography. Recrystallization with toluene and n-heptane yielded compound 196 (6.49 g, 85% yield). The purity was determined by HPLC to be 99.60%. LC/MS (m/z)(M+): 764.31.

### Synthesis of Deuterated Compound 170D

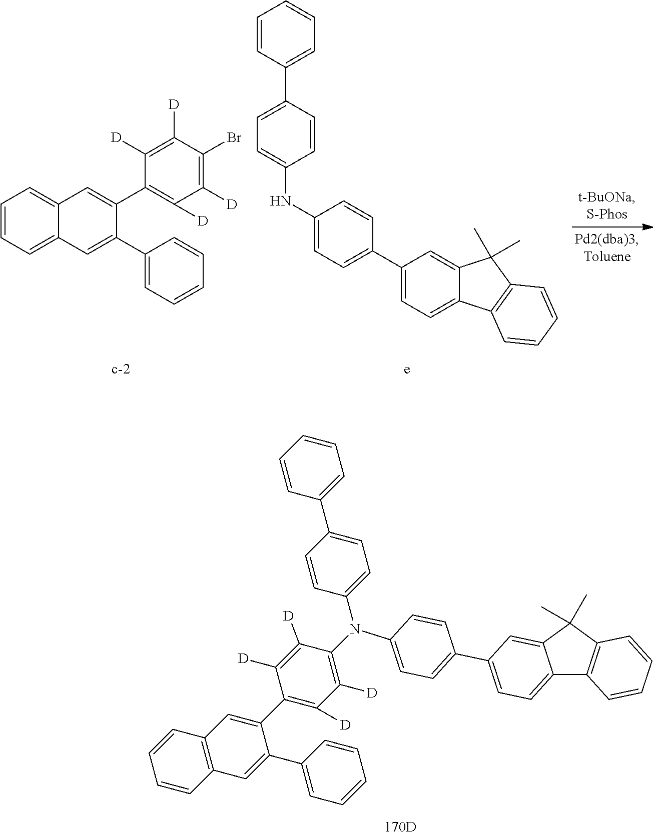

Intermediate Compound c-2 (3.63 g, 10 mmol) and Compound e [N-(4-(9,9-dimethyl-9H-fluoren-2-yl)phenyl)-9,9-dimethyl-9H-fluoren-2-amine](4.77 g, 10 mmol) were added to a reaction flask, followed by 100 mL of toluene. The mixture was heated to reflux for 2 hours under a nitrogen atmosphere, and then cooled. Sodium tert-butoxide (1.44 g, 15 mmol), 2-dicyclohexylphosphino-2′,6′-dimethoxybiphenyl (0.082 g, 0.2 mmol), and bis(dibenzylideneacetone)palladium(0) (0.092 g, 0.1 mmol) were added, and the mixture was heated to reflux for 8-12 hours. Upon completion of the reaction, the reaction was quenched with water and extracted with dichloromethane and water. The organic phase was dried over anhydrous magnesium sulfate, concentrated by rotary evaporation, and purified by silica gel column chromatography. Recrystallization with toluene and n-heptane yielded deuterated compound 170D (4.82 g, 67% yield). The purity was determined by HPLC to be 99.11%. LC/MS (m/z)(M+): 719.34.

### Synthesis of Deuterated Compound 187D

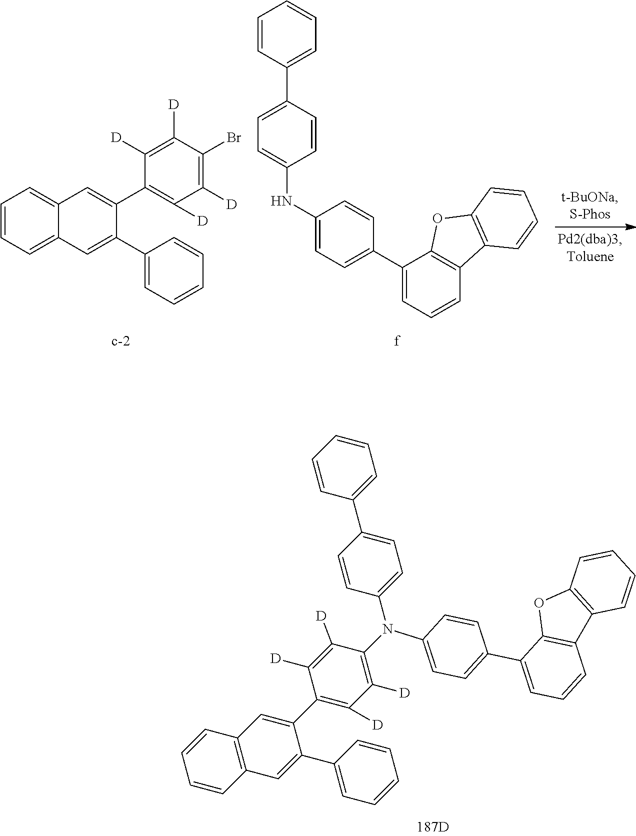

Intermediate Compound c-2 (3.63 g, 10 mmol) and Compound f [N-[4-(dibenzofuran-3-yl)phenyl]-[1,1′-biphenyl]-4-amine](4.12 g, 10 mmol) were added to a reaction flask, followed by 100 mL of toluene. The mixture was heated to reflux for 2 hours under a nitrogen atmosphere, and then cooled. Sodium tert-butoxide (1.44 g, 15 mmol), 2-dicyclohexylphosphino-2′,6′-dimethoxybiphenyl (0.082 g, 0.2 mmol), and bis(dibenzylideneacetone)palladium(0) (0.092 g, 0.1 mmol) were added, and the mixture was heated to reflux for 8-12 hours. Upon completion of the reaction, the reaction was quenched with water and extracted with dichloromethane and water. The organic phase was dried over anhydrous magnesium sulfate, concentrated by rotary evaporation, and purified by silica gel column chromatography. Recrystallization with toluene and n-heptane yielded deuterated compound 187D (4.02 g, 58% yield). The purity was determined by HPLC to be 99.24%. LC/MS (m/z)(M+): 693.29.

### Synthesis of Deuterated Compound 196D

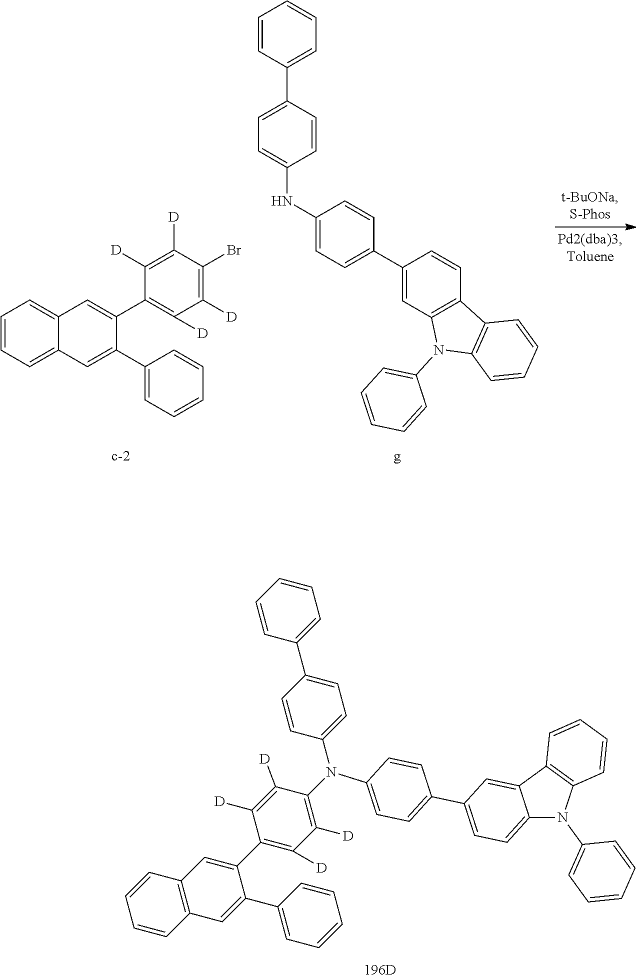 [SMILES: [2H]c1c([2H])c(-c2cc3ccccc3cc2-c2ccccc2)c([2H])c([2H])c1Br ; c1ccc(-c2ccc(Nc3ccc(-c4ccc5c6ccccc6n(-c6ccccc6)c5c4)cc3)cc2)cc1 ; [2H]c1c([2H])c(N(c2ccc(-c3ccccc3)cc2)c2ccc(-c3ccc4c(c3)c3ccccc3n4-c3ccccc3)cc2)c([2H])c([2H])c1-c1cc2ccccc2cc1-c1ccccc1]

Intermediate Compound c-2 (3.63 g, 10 mmol) and Compound g [N-(4-(9-phenyl-9H-carbazol-3-yl)phenyl)-[1,1′-biphenyl]-4-amine](4.86 g, 10 mmol) were added to a reaction flask, followed by 100 mL of toluene. The mixture was heated to reflux for 2 hours under a nitrogen atmosphere, and then cooled. Sodium tert-butoxide (1.44 g, 15 mmol), 2-dicyclohexylphosphino-2′,6′-dimethoxybiphenyl (0.082 g, 0.2 mmol), and bis(dibenzylideneacetone)palladium(0) (0.092 g, 0.1 mmol) were added, and the mixture was heated to reflux for 8-12 hours. Upon completion of the reaction, the reaction was quenched with water and extracted with dichloromethane and water. The organic phase was dried over anhydrous magnesium sulfate, concentrated by rotary evaporation, and purified by silica gel column chromatography. Recrystallization with toluene and n-heptane yielded deuterated compound 196D (5.84 g, 76% yield). The purity was determined by HPLC to be 99.60%. LC/MS (m/z) (M+): 768.34.

### Comparative Compounds

The examples of the present disclosure provide three different comparative compounds, namely compounds D-1, D-2, and D-3. These compounds D-1, D-2, and D-3 were previously synthesized and tested in previous studies. The structural formulas of Compounds D-1, D-2, and D-3 are as follows:

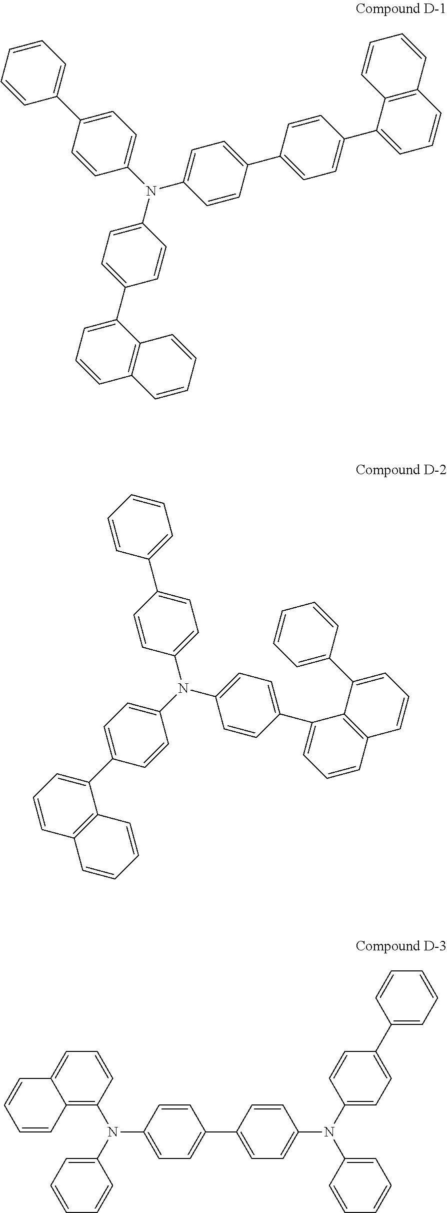 [SMILES: c1ccc(-c2ccc(N(c3ccc(-c4ccc(-c5cccc6ccccc56)cc4)cc3)c3ccc(-c4cccc5ccccc45)cc3)cc2)cc1 ; c1ccc(-c2ccc(N(c3ccc(-c4cccc5ccccc45)cc3)c3ccc(-c4cccc5cccc(-c6ccccc6)c45)cc3)cc2)cc1 ; c1ccc(-c2ccc(N(c3ccccc3)c3ccc(-c4ccc(N(c5ccccc5)c5cccc6ccccc56)cc4)cc3)cc2)cc1]

The physical property data for some of the above synthetic examples and comparative examples are shown in Table 1.1. The data in Table 1.1 were obtained using the simulation software Spartan with the basis set/functional being B3LYP/6-31G**. ROE represents reorganization energy, and it is generally believed that smaller absolute values of ROE indicate a trend toward higher charge carrier mobility.

TABLE 1.1

| Material Name | HOMO | LUMO | ROE |
|---|---|---|---|
| D-1 | −4.90 | −1.22 | −0.140 |
| D-2 | −4.82 | −1.27 | −0.141 |
| D-3 | −4.74 | −1.14 | −0.220 |
| Compound 5 | −4.83 | −1.26 | −0.140 |
| Compound 85 | −4.74 | −0.95 | −0.185 |
| Compound 187 | −4.85 | −1.20 | −0.150 |
| Compound 196 | −4.74 | −1.15 | −0.188 |

### Device Preparation Example A1.1

In this preparation example, aromatic amine Compounds were used as electron barrier layer materials in the preparation of OLED devices. Five different blue OLEDs were prepared using Compound D-1, Compound D-2, Compound 5, Compound 187, and Compound 187D as the electron barrier layer, respectively, namely: comparative Device A1.1A (electron barrier layer material: Compound D-1), Comparative Device A1.1B (electron barrier layer material: Compound D-2), Test Device A1.1A (electron barrier layer material: Compound 5), Test Device A1.1B (electron barrier layer material: Compound 187), and Test Device A1.1C (electron barrier layer material: Compound 187D). The film layers other than the electron barrier layer in Comparative Device A1.1A, Comparative Device A1.1B, and Test Devices A1.1A-A1.1C maintained identical materials and thicknesses.

In this preparation example, the electrode preparation methods and deposition methods for each functional layer are conventional techniques in the field, such as vacuum thermal evaporation or inkjet printing, and will not be elaborated here. Only supplementary explanations of specific process details and testing methods during preparation are provided below:
Forming a pixel driving circuit and anode on a glass substrate;
Depositing a hole injection layer and hole transport layer using an open mask;
Depositing an electron barrier layer and blue light-emitting layer using a fine metal mask (FMM). The thickness of the electron barrier layer is 5 nm. The blue light-emitting layer with the thickness of 20 nm includes a blue light host and blue light guest with a mass ratio of 97:3;
Depositing an electron barrier layer, electron transport layer, and electron injection layer using an open mask;
Depositing a metal cathode using an open mask, finally.
Device information is shown in Table 1.2.

TABLE 1.2

| Device Component | Material | Thickness | |
|---|---|---|---|
| Anode | ITO | 70 | nm |
| Hole Injection | HATCN | 20 | nm |
| Layer | | | |
| Hole Transport | D-3 | 110 | nm |
| Layer | | | |
| Electron barrier | Comparative Device A1.1A (Material: Compound D-1) | 5 | nm |
| Layer | Comparative Device A1.1B (Material: Compound D-2) | | |
| | Test Device A1.1A (Material: Compound 5) | | |
| | Test Device A1.1B (Material: Compound 187) | | |
| | Test Device A1.1C (Material: Compound 187D) | | |
| Blue Light-Emitting | Blue light host: BH; Blue light guest: BD | 20 | nm |
| Layer | | | |
| Hole barrier Layer | TPBI | 10 | nm |
| Electron Transport | BCP/Liq, mass ratio 1:1 | 40 | nm |
| Layer | | | |
| Electron Injection | Yb | 10 | nm |
| Cathode | Mg/Ag alloy, mass ratio 2:8 | 15 | nm |

In this embodiment, except for the materials of the electron barrier layer, the molecular structural formulas of the materials in each layer are as follows:

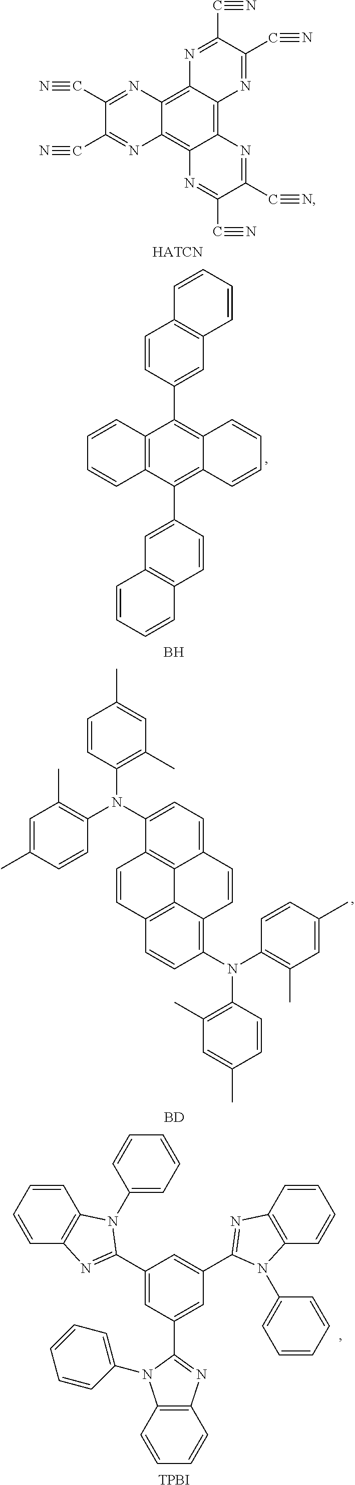

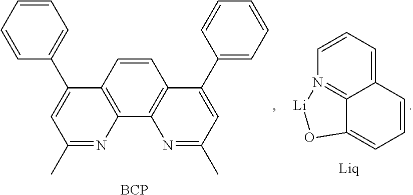

The performance of the fabricated OLED devices was measured, with the performance of Comparative Device A1.1A set as the baseline (100%). The performance data of other OLED devices are summarized in Table 1.3.

TABLE 1.3
Summary of OLED Device Performance

| | | Device | Device |
|---|---|---|---|
| | Voltage | Efficiency | Lifetime |
| Comparative Device A1.1A | 100% | 100% | 100% |
| Comparative Device A1.1B | 100% | 100% | 115% |
| Test Device A1.1A | 96% | 110% | 120% |
| Test Device A1.1B | 98% | 124% | 113% |
| Test Device A1.1C | 98% | 123% | 134% |

Compared to Comparative Devices A1.1A and A1.1B, the devices fabricated using the aromatic amine compounds of the present disclosure (Test Devices A1.1A-A1.1C) exhibit improvements in all performance metrics. This enhancement is attributed to the unique connection angles formed by the 1,8- or 2,3-position linkages at the Ar₃ position of the inventive aromatic amine compounds, which endow them with higher charge carrier mobility and elevated triplet energy (T1), allowing more excitons to recombine in the light-emitting layer and emit light. Additionally, Test Device A1.1C demonstrates significantly improved lifetime compared to Test Device A1.1B. This is due to the C-D (carbon-deuterium) bond, which vibrates at a lower frequency and has a lower zero-point vibrational energy compared to the corresponding C—H (carbon-hydrogen) bond, while their transition state activation energies are similar. Consequently, breaking a C-D bond requires more energy than breaking a C—H bond, making the C-D bond more stable. The superior performance of Test Device A1.1C over Comparative Device A1.1A is also attributed to its high mobility (favoring exciton recombination) and distorted molecular structure (contributing to high T1 levels).

### Device Preparation Example 1.2

This preparation example employs aromatic amine compounds as hole transport layer materials in the preparation of OLED devices. Four different blue OLEDs were prepared using Compound D-3, Compound 85, Compound 196, and Compound 196D as electron barrier layers, namely: Comparative device A1.2A (hole transport layer material: Compound D-3), Test device A1.2A (hole transport layer material: Compound 85), Test device A1.2B (hole transport layer material: Compound 196), and Test device A1.2C (hole transport layer material: Compound 196D). Comparative Device A1.2A and Test Devices A1.2A-A1.2C maintained identical materials and thicknesses for all layers except the hole transport layer (HTL). The electrode preparation methods and deposition methods for each functional layer are conventional techniques in the field, such as vacuum thermal evaporation or inkjet printing, and will not be elaborated here. Only supplementary explanations of specific process details and testing methods during preparation are provided below:
Form a pixel driving circuit and anode on a glass substrate;
Deposit a hole injection layer and hole transport layer using an open mask;
Deposit an electron barrier layer and blue light-emitting layer using a fine metal mask (FMM). The thickness of the electron barrier layer is 5 nm. The blue light-emitting layer with the thickness of 20 nm includes a blue light host and blue light guest with a mass ratio of 97:3;
Deposit an electron barrier layer, electron transport layer, and electron injection layer using an open mask;
Deposit a metal cathode using an open mask, finally.

Device information is shown in Table 1.4.

TABLE 1.4

| Device | | | |
|---|---|---|---|
| Component | Material | Thickness | |
| Anode | ITO | 70 | nm |
| Hole Injection | HATCN | 20 | nm |
| Layer | | | |
| Hole Transport | Comparative Device A1.2A | 110 | nm |
| Layer | (HTL material: Compound D-3) | | |
| | Test Device A1.2A (HTL material: Compound 85) | | |
| | Test Device A1.2B (HTL material: Compound 196) | | |
| | Test Device A1.2C (HTL material: Compound 196D) | | |
| Electron barrier | Compound D-1 | 5 | nm |
| Layer | | | |
| Blue | Blue light host: BH; Blue light guest: BD | 20 | nm |
| Light-Emitting | | | |
| Layer | | | |
| Hole barrier | TPBI | 10 | nm |
| Layer | | | |
| Electron | BCP/Liq, mass ratio 1:1 | 40 | nm |
| Transport | | | |
| Layer | | | |
| Electron | Yb | 10 | nm |
| Injection | | | |
| Cathode | Mg/Ag alloy, mass ratio 2:8 | 15 | nm |

The molecular structural formulas of each layer material, except for the hole transport layer materials, are as follows:

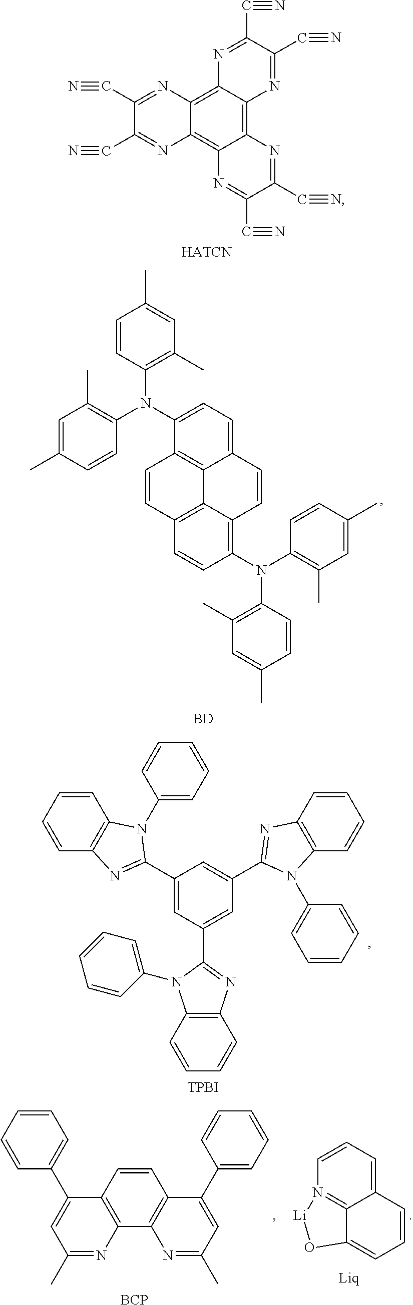

The performance of the fabricated OLED devices was measured, with the performance of the device prepared using Comparative Example 3 set as the baseline. The performance data of other OLED devices are summarized in Table 1.

TABLE 1.5
Summary of OLED Device Performance

| | | Device | Device |
|---|---|---|---|
| | Voltage | Efficiency | Lifetime |
| Comparative Device A1.2A | 100% | 100% | 100% |
| Test Device A1.2A | 100% | 120% | 131% |
| Test Device A1.2B | 100% | 115% | 109% |
| Test Device A1.2C | 99% | 116% | 121% |

Compared to Comparative Device A1.2A, the devices fabricated using the aromatic amine compounds of the present disclosure exhibit higher hole mobility, which facilitates more efficient hole transport to the light-emitting layer, promotes the formation of additional excitons, and enhances device performance.

Using the following synthesis method, Intermediate Compounds III-1 to III-6 were obtained, and then the partial compounds of the exemplary embodiments disclosed in the disclosure were mainly synthesized through Buchwald Harting reaction and Suzuki reaction.

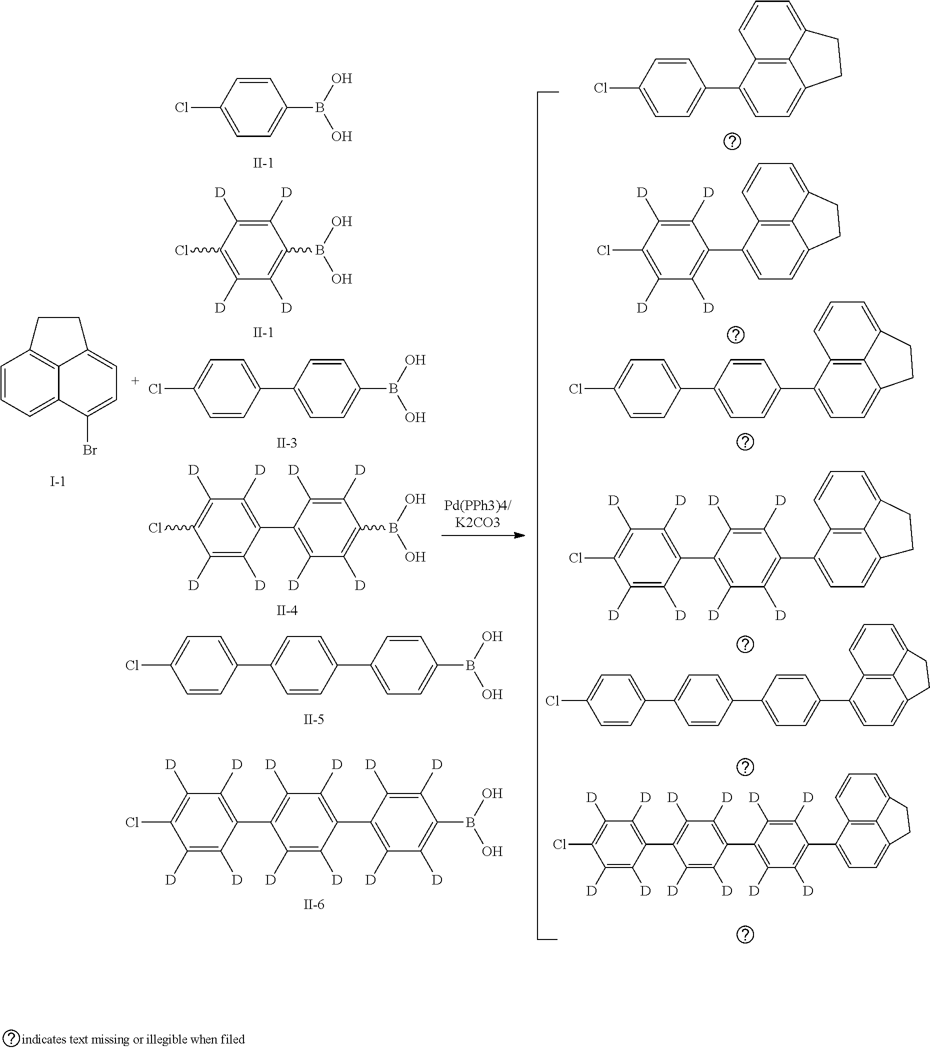

### Intermediate Compound III-1

Under nitrogen protection, Intermediate Compound I-1 (5-bromoacenaphthene) (10 g, 42.90 mmol), Intermediate Compound II-1 (4-chlorophenylboronic acid) (7.72 g, 49.34 mmol), and potassium carbonate (11.86 g, 85.80 mmol) were added to a dried three-necked flask. Toluene (100 mL), ethanol (35 mL), and deionized water (35 mL) were added, and the mixture was heated with stirring until fully dissolved. Tetrakis(triphenylphosphine)palladium(0) (0.74 g, 0.64 mmol) was then introduced, and the reaction was heated to reflux. Progress was monitored by TLC. Upon completion of the reaction of the reaction, the mixture was washed with water and extracted with dichloromethane. The solvent was removed by rotary evaporation. The crude product was purified by silica gel column chromatography with a hexane/dichloromethane (10:1) eluent, and then removes the solvent with a rotary evaporator. Recrystallize the obtained pale yellow solid, filter under suction, and dry under vacuum to yield Intermediate Compound III-1 with a yield of 72% and a purity of 98%; MS: 264.07.

Following the procedure for Intermediate Compound III-1, Intermediate Compounds III-2 to III-6 were synthesized by replacing Compound II-1 with Compounds II-2 to II-6, respectively.

The starting materials, yields, purities, and mass spectrometry (MS) data for Intermediate Compounds III-2 to III-6 are listed in Table 2.1.

TABLE 2.1

| Starting | Starting | |
|---|---|---|
| Material 1 | Material 2 | Product (Intermediate) |
| I | II -2 | III-2; Yield: 65%; Purity: 98.2%; MS: 268.10 |
| | II -3 | III-3; Yield: 69%; Purity: 97.8%; MS: 340.10 |
| | II -4 | III-4; Yield: 66%; Purity: 96.9%; MS: 348.15 |
| | II -5 | III-5; Yield: 71%; Purity: 98.05%; MS: 416.13 |
| | II -6 | II -6 III-6; Yield: 68%; Purity: 97.3%; MS: |
| | | 428.21 |

The Series III Intermediate compounds can be reacted with diarylamine compounds to synthesize the following exemplary compounds disclosed herein:

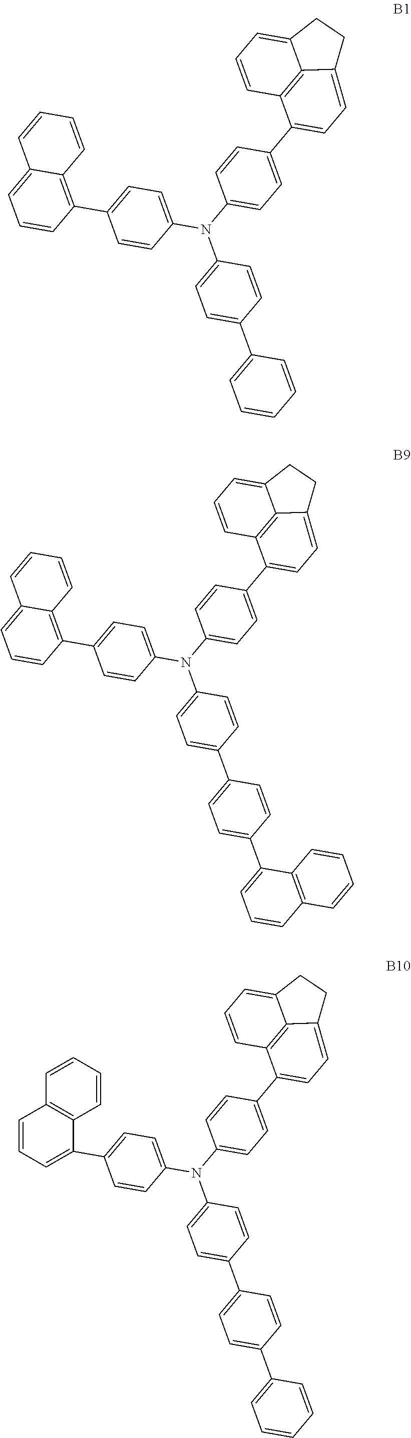 [SMILES: c1ccc(-c2ccc(N(c3ccc(-c4cccc5ccccc45)cc3)c3ccc(-c4ccc5c6c(cccc46)CC5)cc3)cc2)cc1 ; c1ccc2c(-c3ccc(-c4ccc(N(c5ccc(-c6cccc7ccccc67)cc5)c5ccc(-c6ccc7c8c(cccc68)CC7)cc5)cc4)cc3)cccc2c1 ; c1ccc(-c2ccc(-c3ccc(N(c4ccc(-c5cccc6ccccc56)cc4)c4ccc(-c5ccc6c7c(cccc57)CC6)cc4)cc3)cc2)cc1]

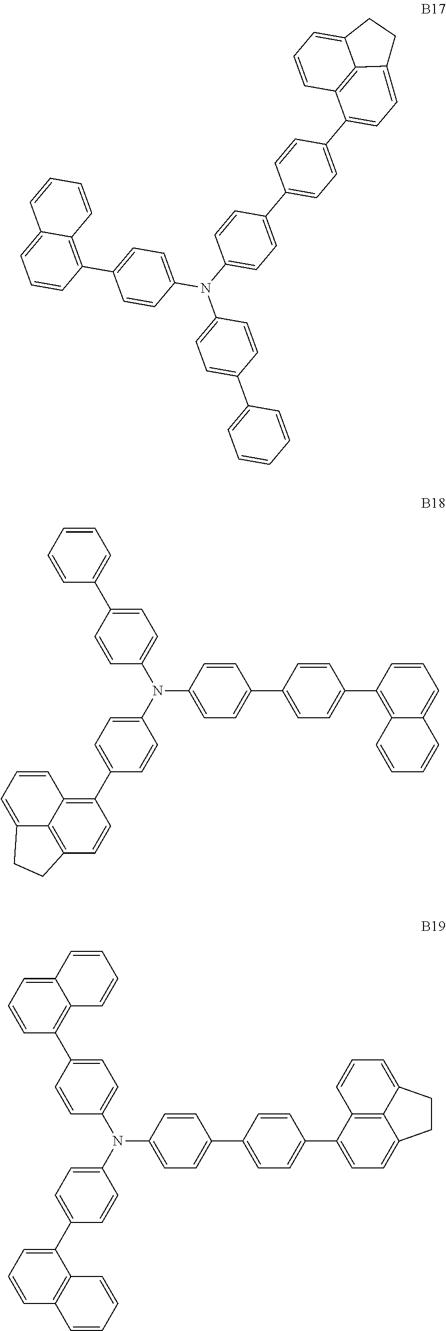 [SMILES: c1ccc(-c2ccc(N(c3ccc(-c4ccc(-c5ccc6c7c(cccc57)CC6)cc4)cc3)c3ccc(-c4cccc5ccccc45)cc3)cc2)cc1 ; c1ccc(-c2ccc(N(c3ccc(-c4ccc(-c5cccc6ccccc56)cc4)cc3)c3ccc(-c4ccc5c6c(cccc46)CC5)cc3)cc2)cc1 ; c1ccc2c(-c3ccc(N(c4ccc(-c5ccc(-c6ccc7c8c(cccc68)CC7)cc5)cc4)c4ccc(-c5cccc6ccccc56)cc4)cc3)cccc2c1]

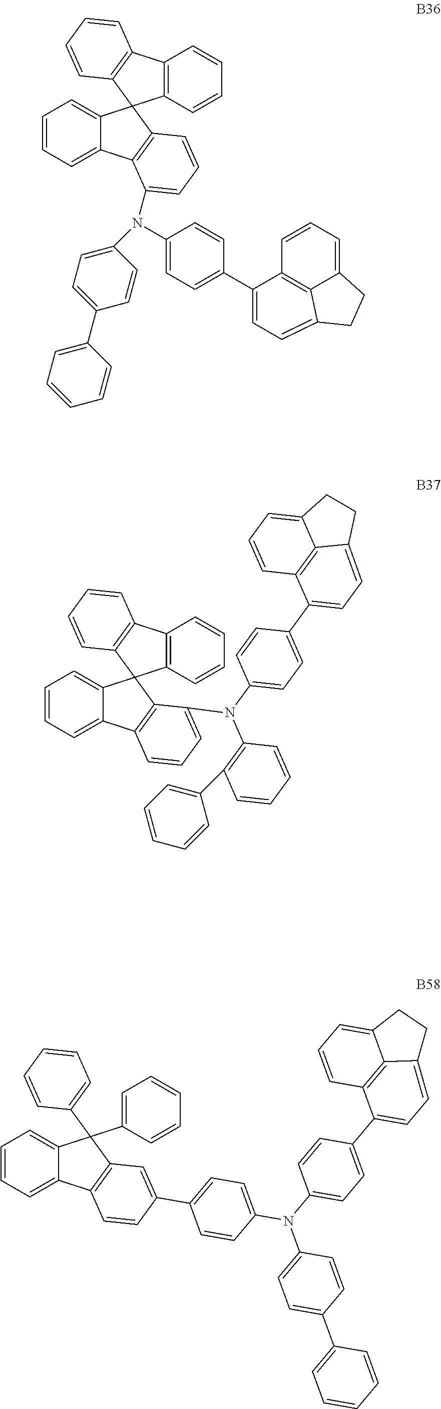 [SMILES: c1ccc(-c2ccc(N(c3ccc(-c4ccc5c6c(cccc46)CC5)cc3)c3cccc4c3-c3ccccc3C43c4ccccc4-c4ccccc43)cc2)cc1 ; c1ccc(-c2ccccc2N(c2ccc(-c3ccc4c5c(cccc35)CC4)cc2)c2cccc3c2C2(c4ccccc4-c4ccccc42)c2ccccc2-3)cc1 ; c1ccc(-c2ccc(N(c3ccc(-c4ccc5c(c4)C(c4ccccc4)(c4ccccc4)c4ccccc4-5)cc3)c3ccc(-c4ccc5c6c(cccc46)CC5)cc3)cc2)cc1]

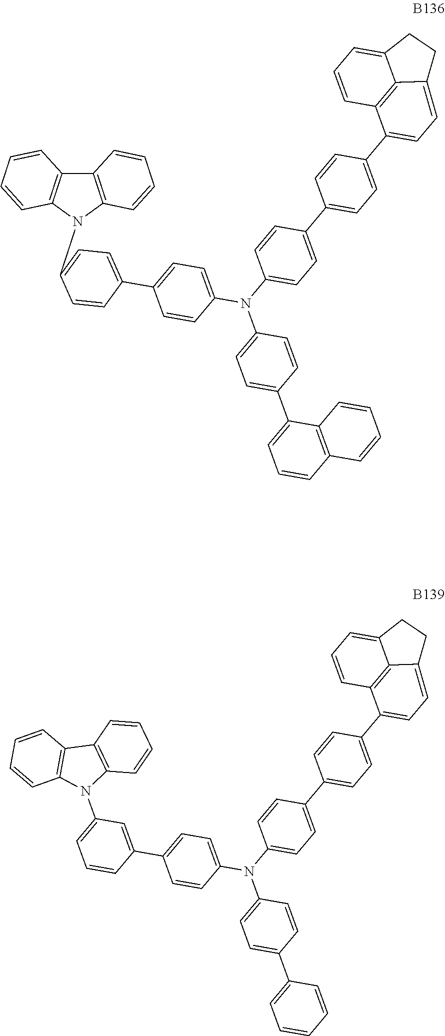 [SMILES: c1ccc2c(-c3ccc(N(c4ccc(-c5ccc(-c6ccc7c8c(cccc68)CC7)cc5)cc4)c4ccc(-c5ccc(-n6c7ccccc7c7ccccc76)cc5)cc4)cc3)cccc2c1 ; c1ccc(-c2ccc(N(c3ccc(-c4ccc(-c5ccc6c7c(cccc57)CC6)cc4)cc3)c3ccc(-c4cccc(-n5c6ccccc6c6ccccc65)c4)cc3)cc2)cc1]

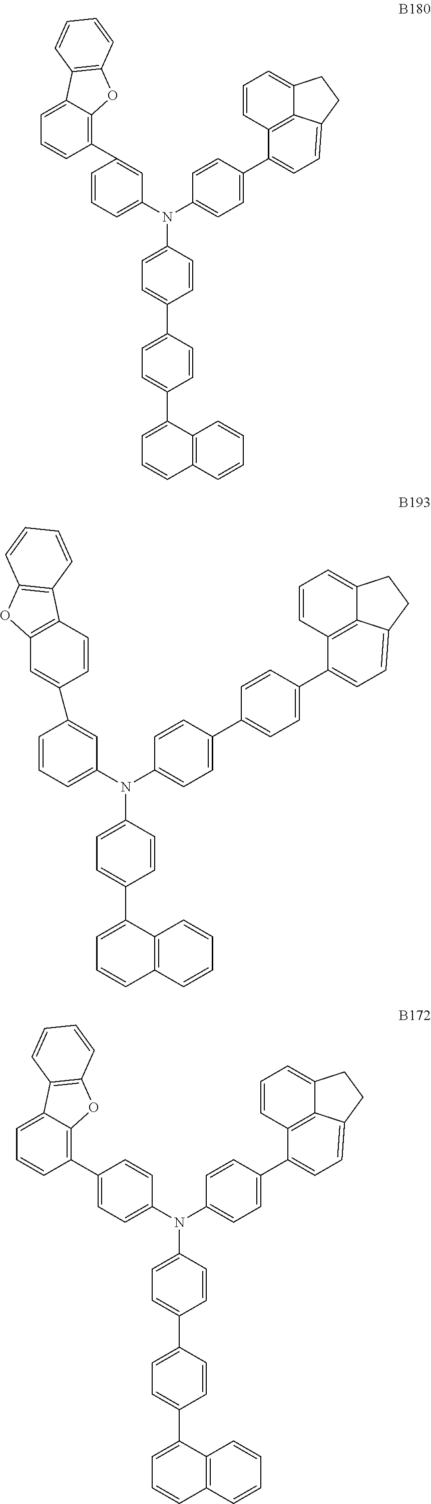 [SMILES: c1cc(-c2cccc3c2oc2ccccc23)cc(N(c2ccc(-c3ccc(-c4cccc5ccccc45)cc3)cc2)c2ccc(-c3ccc4c5c(cccc35)CC4)cc2)c1 ; c1cc(-c2ccc3c(c2)oc2ccccc23)cc(N(c2ccc(-c3ccc(-c4ccc5c6c(cccc46)CC5)cc3)cc2)c2ccc(-c3cccc4ccccc34)cc2)c1 ; c1ccc2c(-c3ccc(-c4ccc(N(c5ccc(-c6ccc7c8c(cccc68)CC7)cc5)c5ccc(-c6cccc7c6oc6ccccc67)cc5)cc4)cc3)cccc2c1]

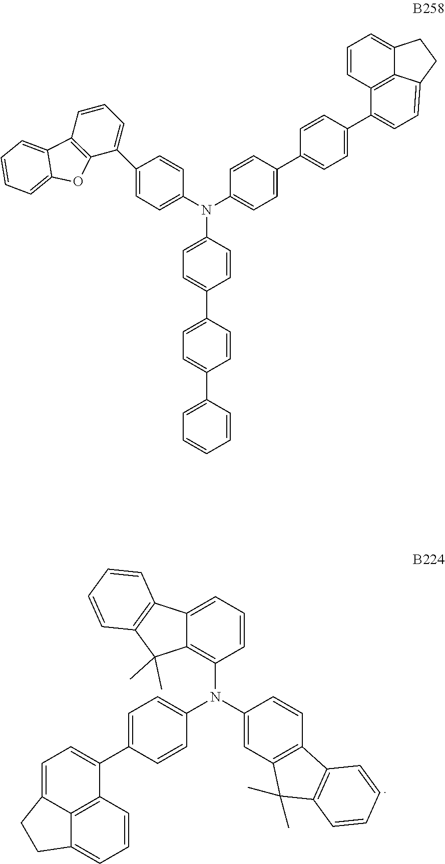 [SMILES: c1ccc(-c2ccc(-c3ccc(N(c4ccc(-c5ccc(-c6ccc7c8c(cccc68)CC7)cc5)cc4)c4ccc(-c5cccc6c5oc5ccccc56)cc4)cc3)cc2)cc1 ; CC1(C)c2ccccc2-c2ccc(N(c3ccc(-c4ccc5c6c(cccc46)CC5)cc3)c3cccc4c3C(C)(C)c3ccccc3-4)cc21]

When synthesizing the aforementioned exemplary compounds, the following diarylamines can be employed as starting materials:

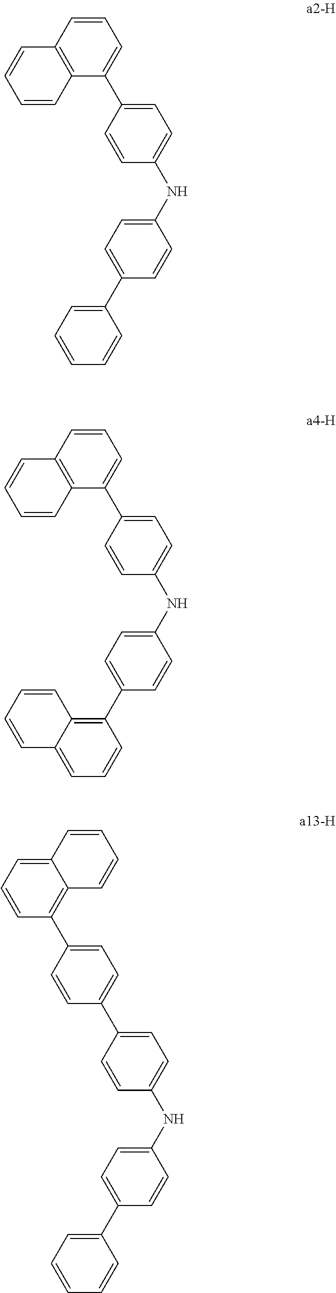 [SMILES: c1ccc(-c2ccc(Nc3ccc(-c4cccc5ccccc45)cc3)cc2)cc1 ; c1ccc2c(-c3ccc(Nc4ccc(-c5cccc6ccccc56)cc4)cc3)cccc2c1 ; c1ccc(-c2ccc(Nc3ccc(-c4ccc(-c5cccc6ccccc56)cc4)cc3)cc2)cc1]

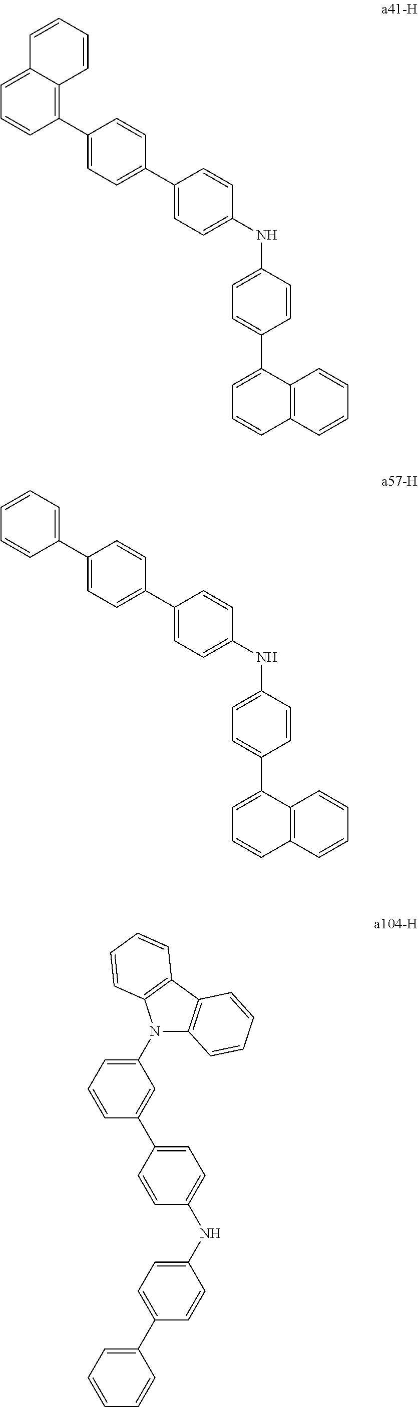 [SMILES: c1ccc2c(-c3ccc(Nc4ccc(-c5ccc(-c6cccc7ccccc67)cc5)cc4)cc3)cccc2c1 ; c1ccc(-c2ccc(-c3ccc(Nc4ccc(-c5cccc6ccccc56)cc4)cc3)cc2)cc1 ; c1ccc(-c2ccc(Nc3ccc(-c4cccc(-n5c6ccccc6c6ccccc65)c4)cc3)cc2)cc1]

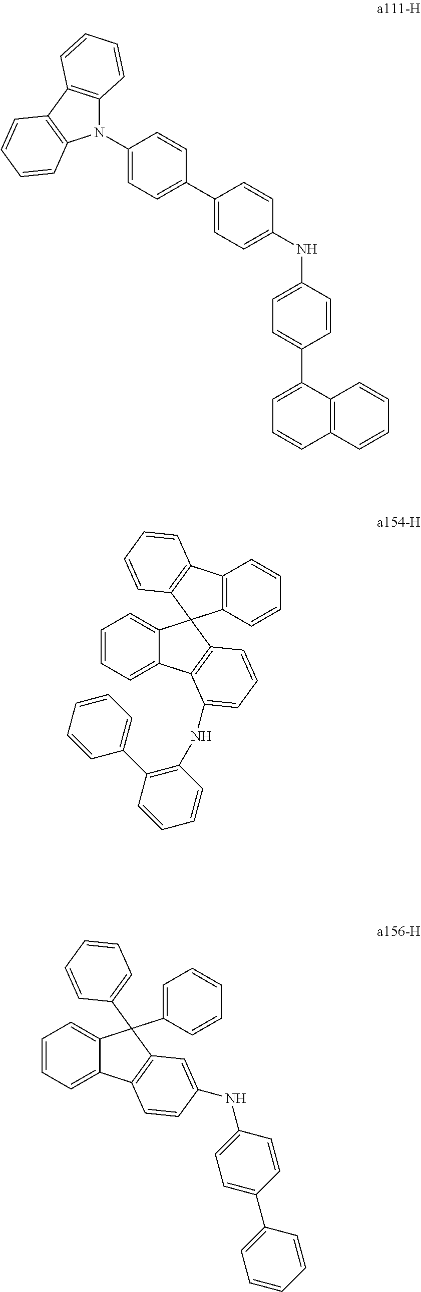 [SMILES: c1ccc2c(-c3ccc(Nc4ccc(-c5ccc(-n6c7ccccc7c7ccccc76)cc5)cc4)cc3)cccc2c1 ; c1ccc(-c2ccccc2Nc2cccc3c2-c2ccccc2C32c3ccccc3-c3ccccc32)cc1 ; c1ccc(-c2ccc(Nc3ccc4c(c3)C(c3ccccc3)(c3ccccc3)c3ccccc3-4)cc2)cc1]

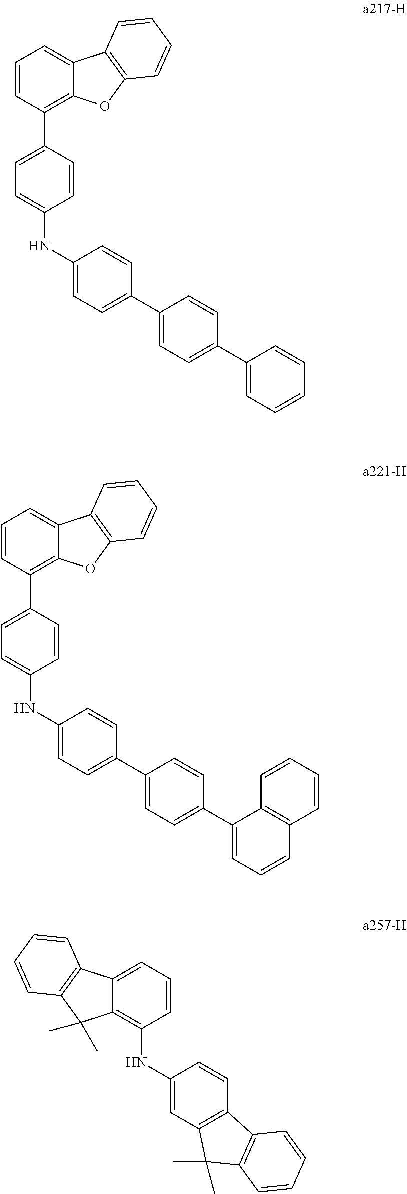 [SMILES: c1ccc(-c2ccc(-c3ccc(Nc4ccc(-c5cccc6c5oc5ccccc56)cc4)cc3)cc2)cc1 ; c1ccc2c(-c3ccc(-c4ccc(Nc5ccc(-c6cccc7c6oc6ccccc67)cc5)cc4)cc3)cccc2c1 ; CC1(C)c2ccccc2-c2ccc(Nc3cccc4c3C(C)(C)c3ccccc3-4)cc21]

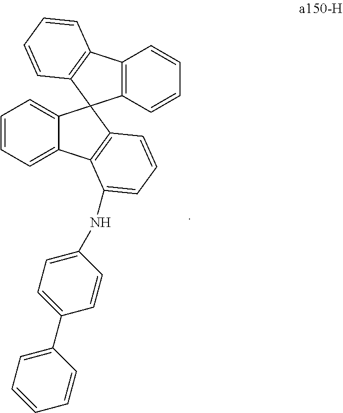 [SMILES: c1ccc(-c2ccc(Nc3cccc4c3-c3ccccc3C43c4ccccc4-c4ccccc43)cc2)cc1]

### Synthesis of Compound B1

Intermediate Compound III-1 (5.58 g, 21 mmol) and N-(4-(1-naphthyl)phenyl)-4-biphenylamine (the corresponding diarylamine a2-H of Intermediate a2) (7.43 g, 20 mmol) were dissolved in toluene under nitrogen protection and stirred under reflux. Then, sodium tert-butoxide, SPhos, and Pd₂(dba)₃ were sequentially added, and the mixture was heated under reflux overnight. After completion, the reaction was cooled to room temperature, and distilled water was added to the reaction mixture. The solution was then extracted with dichloromethane (DCM). The organic layer was dried over magnesium sulfate, and the solvent was removed using a rotary evaporator. The crude product was purified by column chromatography, and the solvent was evaporated again. The resulting solid was recrystallized from a mixture of toluene/n-heptane to afford compound B1 with: Purity: 99.63%; Yield: 87%; MS: 599.26; Molecular weight: C46H33N.

Referring to the synthesis method of Compound B1, Compound B9, Compound B10, Compound B18, Compound B37, Compound B58, and Compound B172 were synthesized by replacing diarylamine a2-H with diarylamines a41-H, a57-H, a13-H, a154-H, a156-H, and a221-H, respectively. The starting materials, synthetic yields, purities, and MS data for compounds B9, B10, B18, B37, B58, and B172 are listed in Table 2.2.

TABLE 2.2

| Staring | Staring | |
|---|---|---|
| Material 1 | Material 2 | Produce |
| III-1 | a41-H | B9; Yield: 88.5%; Purity: 99.67%; MS: 725.31 |
| | a57-H | B10; Yield: 86%; Purity: 99.82%; MS: 675.29 |
| | a13-H | B18; Yield: 87.1%; Purity: 99.77%; MS: 675.30 |
| | a154-H | B37; Yield: 79%; Purity: 99.8%; MS: 711.29 |
| | a156-H | B58; Yield: 83%; Purity: 99.56%; MS: 713.31 |
| | a221-H | B172; Yield: 81.4%; Purity: 99.64%; MS: 765.30 |

### Synthesis of Compound B17

Intermediate Compound III-3 (7.16 g, 21 mmol) and N-(4-(1-naphthyl)phenyl)-4-biphenylamine (the corresponding diarylamine a2-H of Intermediate a2) (7.43 g, 20 mmol) were dissolved in toluene under nitrogen protection and stirred under reflux. Then, sodium tert-butoxide, SPhos, and Pd₂(dba)₃ were sequentially added, and the mixture was heated under reflux overnight. After completion, the reaction was cooled to room temperature, and distilled water was added to the reaction mixture. The solution was then extracted with dichloromethane (DCM). The organic layer was dried over magnesium sulfate, and the solvent was removed using a rotary evaporator. The crude product was purified by column chromatography, and the solvent was evaporated again. The resulting solid was recrystallized from a mixture of toluene/n-heptane to afford Compound B17 with: Purity: 99.7%; Yield: 86%; MS: 675.29; Molecular weight: C52H37N.

Referring to the synthesis method of Compound B17, Compound B19, Compound B136, Compound B139, and Compound B258 were synthesized by replacing diarylamine a2-H with diarylamines a4-H, a111-H, a104-H, and a217-H, respectively. The starting materials, synthetic yields, purities, and MS data for compounds B19, B136, B139, and B258 are listed in Table 2.2.

TABLE 2.3

| Staring | Staring | |
|---|---|---|
| Material 1 | Material 2 | Product |
| III-3 | a4-H | B19; Yield: 83%; Purity: 99.51%; MS: 725.30 |
| | a111-H | B136; Yield: 78%; Purity: 99.63%; MS: 840.35 |
| | a104-H | B139; Yield: 79.5%; Purity: 99.6%; MS: 790.34 |
| | a217-H | B258; Yield: 78.4%; Purity: 99.59%; MS: 791.32 |

### Synthesis of Partially Deuterated Compound B17D

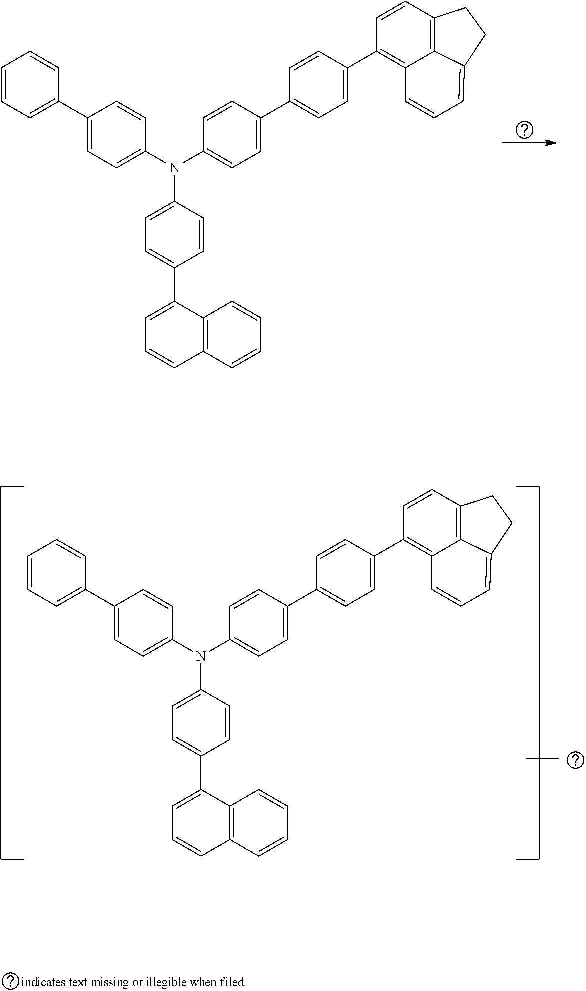

Under nitrogen protection, Compound B17 (6.75 g, 10 mmol), deuterated benzene (89 mL, 1 mol), trifluoromethanesulfonic acid (9.43 g, 60 mmol), and 1,4-dioxane (500 mL) were added to a three-necked flask. The reaction was carried out at 90° C. overnight, with progress monitored by TLC. Upon completion, the mixture was cooled to room temperature. The reaction solution was extracted with dichloromethane and deionized water. The organic phase was washed with water until neutral, and then dried over anhydrous magnesium sulfate. The solvent was removed using a rotary evaporator. The product was recrystallized from a mixture of dichloromethane and n-heptane to yield a pale yellow solid, Compound B17D. Compound B17D. Yield: 43%; Purity: 99.53%; MS [M+H]+: 708.29. All hydrogens attached to SP²-hybridized carbons in Compound B17 are replaced with deuterium to form Compound B17D.

In this embodiment of the present disclosure, the above-mentioned exemplary compounds were also compared in properties with reference compounds. The structures of the reference compounds are as follows:

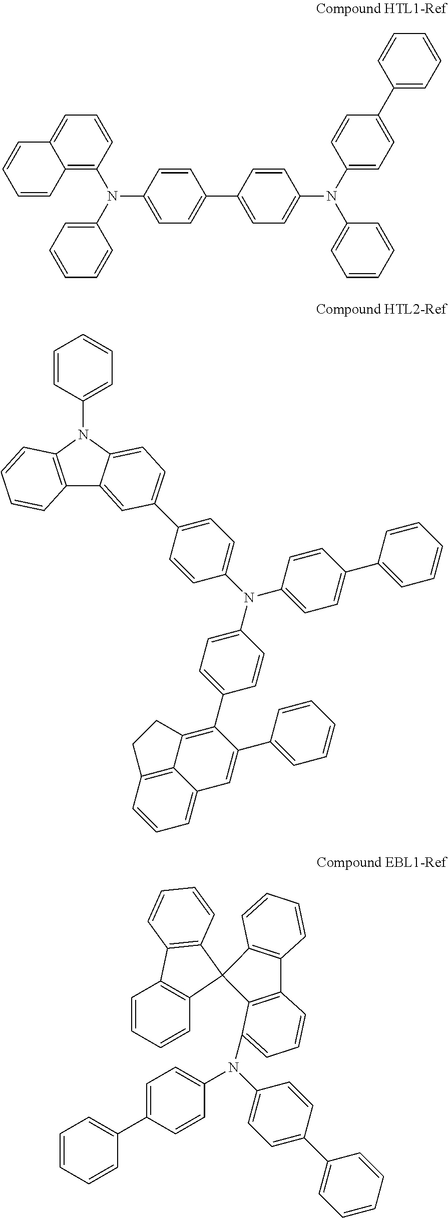 [SMILES: c1ccc(-c2ccc(N(c3ccccc3)c3ccc(-c4ccc(N(c5ccccc5)c5cccc6ccccc56)cc4)cc3)cc2)cc1 ; c1ccc(-c2ccc(N(c3ccc(-c4ccc5c(c4)c4ccccc4n5-c4ccccc4)cc3)c3ccc(-c4c(-c5ccccc5)cc5cccc6c5c4CC6)cc3)cc2)cc1 ; c1ccc(-c2ccc(N(c3ccc(-c4ccccc4)cc3)c3cccc4c3C3(c5ccccc5-c5ccccc53)c3ccccc3-4)cc2)cc1]

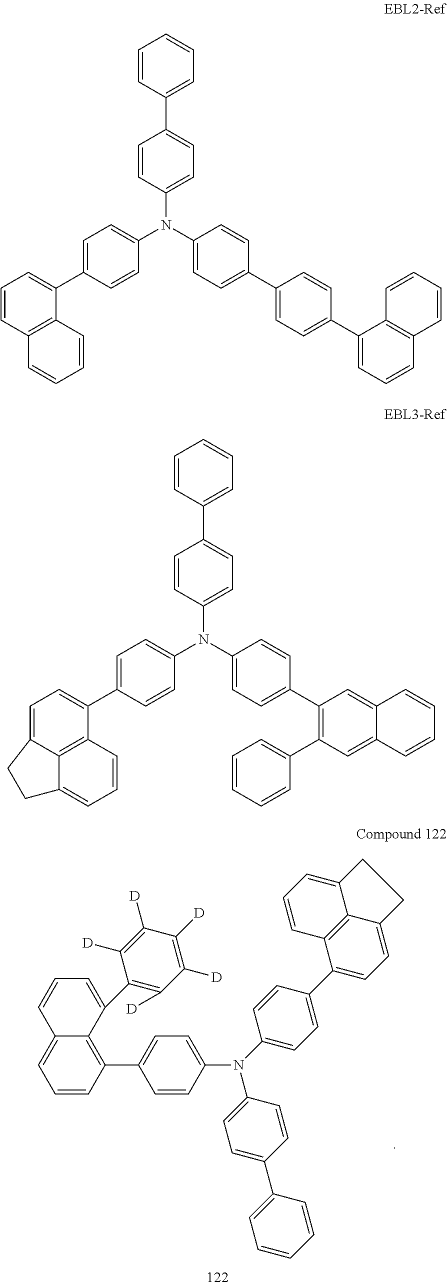 [SMILES: c1ccc(-c2ccc(N(c3ccc(-c4ccc(-c5cccc6ccccc56)cc4)cc3)c3ccc(-c4cccc5ccccc45)cc3)cc2)cc1 ; c1ccc(-c2ccc(N(c3ccc(-c4cc5ccccc5cc4-c4ccccc4)cc3)c3ccc(-c4ccc5c6c(cccc46)CC5)cc3)cc2)cc1 ; [2H]c1c([2H])c([2H])c(-c2cccc3cccc(-c4ccc(N(c5ccc(-c6ccccc6)cc5)c5ccc(-c6ccc7c8c(cccc68)CC7)cc5)cc4)c23)c([2H])c1[2H]]

The physicochemical properties of the aforementioned reference compounds and Compound B10, Compound B17, Compound B19, and Compound B224 were simulated using the simulation software Spartan (basis set/functional: B3LYP/6-31G**). Results are shown in Table 2.4. Here, ROE represents reorganization energy, and it is generally believed that smaller absolute values of ROE indicate a trend toward higher charge carrier mobility.

TABLE 2.4

| Compound | HOMO (eV) | LUMO (eV) | ROE (eV) |
|---|---|---|---|
| HTL1-Ref | −4.74 | −1.14 | −0.22 |
| HTL2-Ref | −4.74 | −1.15 | −0.19 |
| EBL1-Ref | −4.94 | −0.99 | −0.25 |
| EBL2-Ref | −4.90 | −1.22 | −0.14 |
| EBL3-Ref | −4.81 | −1.15 | −0.16 |
| Compound B10 | −4.84 | −1.17 | −0.13 |
| Compound B17 | −4.86 | −1.14 | −0.13 |
| Compound B18 | −4.83 | −1.20 | −0.153 |
| Compound B19 | −4.88 | −1.15 | −0.14 |
| Compound B224 | −4.71 | −0.97 | −0.18 |
| Compound 122 | −4.76 | −1.26 | −0.157 |

### Device Preparation and Test Example 2.1

Multiple organic electroluminescent devices were fabricated using the following method, and their driving voltage, luminous efficiency, and lifetime were tested. The preparation process of the organic electroluminescent devices is as follows:

The pre-prepared ITO substrate was cleaned and dried, and the ITO substrate had an ITO layer as the anode; on the anode, a hole injection layer, a hole transport layer, an electron barrier layer, an organic light-emitting layer, a hole barrier layer, an electron transport layer, an electron injection layer, a cathode and a cover layer were sequentially formed by evaporation deposition.

The material of the hole injection layer was a mixture of Compound HT2.1 and Compound PD2.1, with a thickness of 20 nm; the doping ratio of Compound PD2.1 was 2%;
The material of the hole transport layer was Compound HT2.1, with a thickness of 110 nm;
The material of the organic light-emitting layer was a mixture of Compound BH2.1 and Compound BD2.1, the doping ratio of Compound BD2.1 was 4%, with a thickness of 20 nm;
The material of the hole barrier layer was Compound HB2.1, with a thickness of 10 nm;
The material of the electron transport layer was Compound ET2.1, with a thickness of 30 nm;
The material of the electron injection layer was Yb, with a thickness of 5 nm;
The material of the cathode was Mg:Ag (2:8) alloy, with a thickness of 12 nm;
The material of the cover layer was CP2.1, with a thickness of 70 nm.

In the Device Preparation and Test Example 2.1, according to the different materials used for the electron barrier layer of the organic electroluminescent device, the organic electroluminescent devices were divided into Reference Device B2.1 (the material of the electron barrier layer was Compound EBL1-Ref), Test Device B2.1A (the material of the electron barrier layer was Compound B37), Test Device B2.1B (the material of the electron barrier layer was Compound B160), and Test Device B2.1C (the material of the electron barrier layer was Compound B193). The differences between Reference Device B2.1, Test Device B2.1A, Test Device B2.1B and Test Device B2.1C were only in the materials of the electron barrier layers, while the materials and thicknesses of the other film layers were the same. The thickness of the electron barrier layer was 10 nm.

Structural formulas of the materials are as follows:

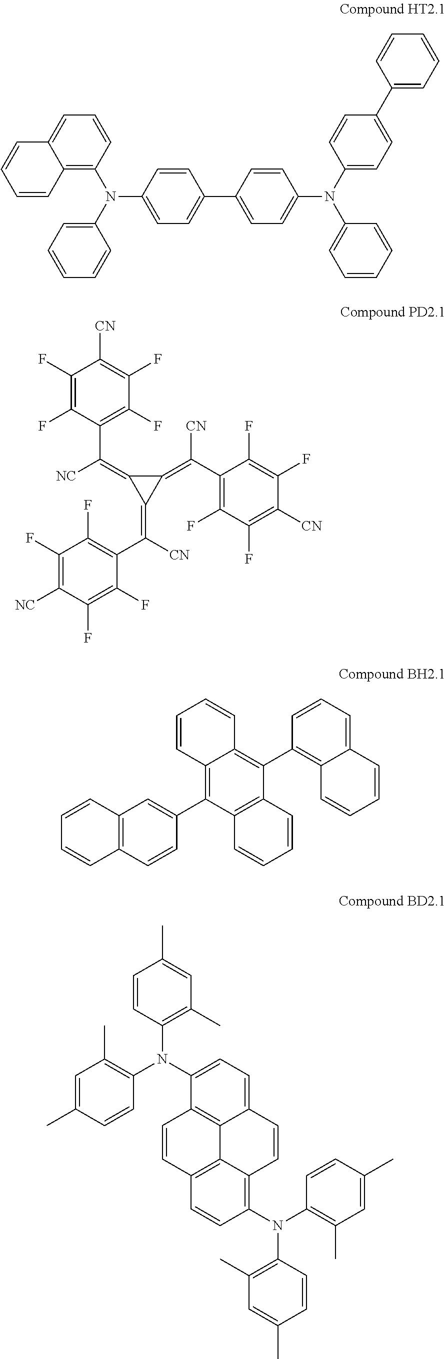

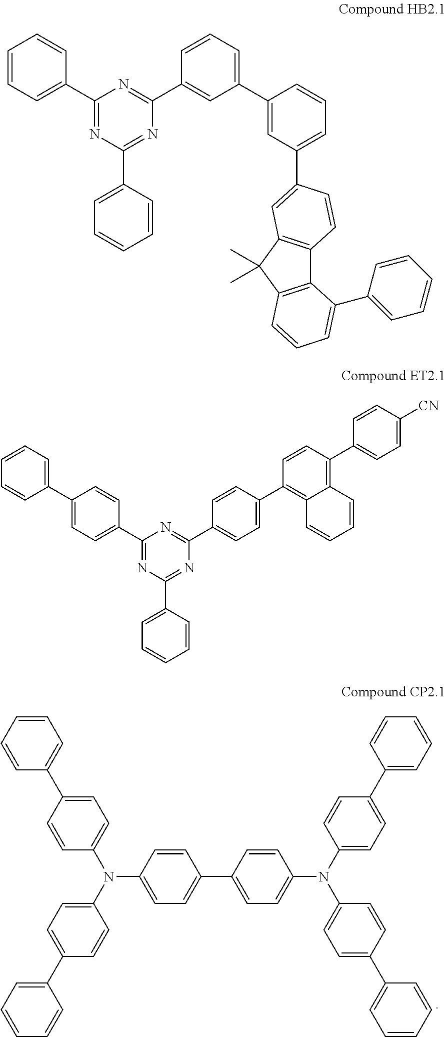 [SMILES: CC1(C)c2cc(-c3cccc(-c4cccc(-c5nc(-c6ccccc6)nc(-c6ccccc6)n5)c4)c3)ccc2-c2c(-c3ccccc3)cccc21 ; N#Cc1ccc(-c2ccc(-c3ccc(-c4nc(-c5ccccc5)nc(-c5ccc(-c6ccccc6)cc5)n4)cc3)c3ccccc23)cc1 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1]

The driving voltage, luminous efficiency, and lifetime of Reference Device B2.1 and Test Devices B2.1A, B2.1B, B2.1C were tested. Results are shown in Table 2.5, where the test data for each device were normalized against Reference Device B2.1. Here, the lifetime refers to T95 lifetime, measured at room temperature with a current density of 15 mA/cm²; the efficiency refers to current efficiency, measured at a current density of 15 mA/cm².

TABLE 2.5

| Device | Voltage | Efficiency | Lifetime |
|---|---|---|---|
| Reference Device B2.1 | 100% | 100% | 100% |
| Test Device B2.1A | 96% | 113% | 120% |
| Test Device B2.1B | 98% | 121% | 106% |
| Test Device B2.1C | 97% | 117% | 110% |

Compared to Reference Device B2.1, Test Devices B2.1A-B2.1C exhibit higher efficiency and longer lifetime. This is attributed to the introduction of cycloalkylnaphthalene in the electron barrier layer (EBL) materials of test devices B2.1A-B2.1C. The cycloalkylnaphthalene moiety enhances charge carrier mobility and provides a relatively shallower energy level, while its distorted molecular structure reduces quenching and crystallization caused by intermolecular stacking, thereby improving device efficiency and lifetime.

### Device Preparation and Test Example 2.2

Multiple organic electroluminescent devices were fabricated using the following method, and their driving voltage, luminous efficiency, and lifetime were tested. The preparation process of the organic electroluminescent devices is as follows:

The pre-prepared ITO substrate was cleaned and dried, and the ITO substrate had an ITO layer as the anode; on the anode, a hole injection layer, a hole transport layer, an electron barrier layer, an organic light-emitting layer, a hole barrier layer, an electron transport layer, an electron injection layer, a cathode and a cover layer were sequentially formed by evaporation deposition.

The material of the hole injection layer was a mixture of Compound HT2.1 and Compound PD2.1, with a thickness of 20 nm; the doping ratio of Compound PD2.1 was 2%;
The material of the hole transport layer was Compound HT2.1, with a thickness of 110 nm;
The material of the organic light-emitting layer was a mixture of Compound BH2.1 and Compound BD2.1, the doping ratio of Compound BD2.1 was 4%, with a thickness of 20 nm;
The material of the hole barrier layer was Compound HB2.1, with a thickness of 10 nm;
The material of the electron transport layer was Compound ET2.1, with a thickness of 30 nm;
The material of the electron injection layer was Yb, with a thickness of 5 nm;
The material of the cathode was Mg:Ag (2:8) alloy, with a thickness of 12 nm;
The material of the cover layer was CP2.1, with a thickness of 70 nm.

In the Device Preparation and Test Example 2.2, according to the different materials used for the electron barrier layer of the organic electroluminescent device, the organic electroluminescent devices were divided into Reference Device B2.2 (the material of the electron barrier layer was Compound EBL2-Ref), Test Device B2.2A (the material of the electron barrier layer was Compound B1), Test Device B2.2B (the material of the electron barrier layer was Compound B10), Test Device B2.2C (the material of the electron barrier layer was Compound B17), and Test Device B2.2D (the material of the electron barrier layer was Compound B19). The differences between Reference Device B2.2, Test Device B2.2A, Test Device B2.2B, Test Device B2.2C and Test Device 2.2D were only in the materials of the electron barrier layers, while the materials and thicknesses of the other film layers were the same. The thickness of the electron barrier layer was 10 nm.

The driving voltage, luminous efficiency, and lifetime of Reference Device B2.2 and Test Devices B2.2A, B2.2B, B2.2C and B2.2D were tested. Results are shown in Table 2.6, where the test data for each device were normalized against Reference Device B2.2. Here, the lifetime refers to T95 lifetime, measured at room temperature with a current density of 15 mA/cm²; the efficiency refers to current efficiency, measured at a current density of 15 mA/cm².

TABLE 2.6

| Device | Voltage | Efficiency | Lifetime |
|---|---|---|---|
| Reference Device B2.2 | 100% | 100% | 100% |
| Test Device B2.2A | 94% | 113% | 104% |
| Test Device B2.2B | 96% | 125% | 111% |
| Test Device B2.2C | 96% | 123% | 117% |
| Test Device B2.2D | 98% | 118 | 113% |

Compared to Reference Device B2.2, Test Devices B2.2A-B2.2D exhibit higher efficiency and longer lifetime. This is attributed to the introduction of cycloalkylnaphthalene in the electron barrier layer (EBL) materials of Test Devices B2.2A-B2.2D. The cycloalkylnaphthalene moiety enhances charge carrier mobility and provides a relatively shallower energy level, while its distorted molecular structure reduces quenching and crystallization caused by intermolecular stacking, thereby improving device efficiency and lifetime.

### Device Preparation and Test Example 2.3

Multiple organic electroluminescent devices were fabricated using the following method, and their driving voltage, luminous efficiency, and lifetime were tested. The preparation process of the organic electroluminescent devices is as follows:

The pre-prepared ITO substrate was cleaned and dried, and the ITO substrate had an ITO layer as the anode; on the anode, a hole injection layer, a hole transport layer, an electron barrier layer, an organic light-emitting layer, a hole barrier layer, an electron transport layer, an electron injection layer, a cathode and a cover layer were sequentially formed by evaporation deposition.

The material of the hole injection layer was a mixture of Compound HT2.1 and Compound PD2.1, with a thickness of 20 nm; the doping ratio of Compound PD2.1 was 2%;
The material of the hole transport layer was Compound HT2.1, with a thickness of 110 nm;
The material of the organic light-emitting layer was a mixture of Compound BH2.1 and Compound BD2.1, the doping ratio of Compound BD2.1 was 4%, with a thickness of 20 nm;
The material of the hole barrier layer was Compound HB2.1, with a thickness of 10 nm;
The material of the electron transport layer was Compound ET2.1, with a thickness of 30 nm;
The material of the electron injection layer was Yb, with a thickness of 5 nm;
The material of the cathode was Mg:Ag (2:8) alloy, with a thickness of 12 nm;
The material of the cover layer was CP2.1, with a thickness of 70 nm.

In the Device Preparation and Test Example 2.3, according to the different materials used for the electron barrier layer of the organic electroluminescent device, the organic electroluminescent devices were divided into Reference Device B2.3 (the material of the electron barrier layer was Compound EBL3-Ref), and Test Device B2.3A (the material of the electron barrier layer was Compound B17). The differences between Reference Device B2.3, and Test Device B2.3A were only in the materials of the electron barrier layers, while the materials and thicknesses of the other film layers were the same. The thickness of the electron barrier layer was 10 nm.

The driving voltage, luminous efficiency, and lifetime of Reference Device B2.3 and Test Device B2.3A were tested. Results are shown in Table 2.7, where the test data for each device were normalized against Reference Device B2.3. Here, the lifetime refers to T95 lifetime, measured at room temperature with a current density of 15 mA/cm²; the efficiency refers to current efficiency, measured at a current density of 15 mA/cm².

TABLE 2.7

| Device | Voltage | Efficiency | Lifetime |
|---|---|---|---|
| Reference Device B2.3 | 100% | 100% | 100% |
| Test Device B2.3A | 98% | 118% | 106% |

Compared to Reference Device B2.3, Test Device B2.3A exhibited lower driving voltage, higher efficiency, and longer lifetime. Compared to Compound EBL3-Ref, Compound B17 has a deeper HOMO energy level, which facilitates more efficient hole injection into the organic light-emitting layer. Additionally, Compound B17 has higher mobility than Compound EBL3-Ref, contributing to the reduction in driving voltage.

### Device Preparation and Test Example 2.4

Multiple organic electroluminescent devices were fabricated using the following method, and their driving voltage, luminous efficiency, and lifetime were tested. The preparation process of the organic electroluminescent devices is as follows:

The pre-prepared ITO substrate was cleaned and dried, and the ITO substrate had an ITO layer as the anode; on the anode, a hole injection layer, a hole transport layer, an electron barrier layer, an organic light-emitting layer, a hole barrier layer, an electron transport layer, an electron injection layer, a cathode and a cover layer were sequentially formed by evaporation deposition.

The material of the hole injection layer is a mixture of a hole-transport compound and Compound PD2.1, with a thickness of 20 nm; the doping ratio of Compound PD2.1 is 2%;
The material of the hole transport layer is a hole-transport compound, with a thickness of 110 nm;
The material of the electron barrier layer is Compound EBL1-Ref, with a thickness of 10 nm;
The material of the organic light-emitting layer was a mixture of Compound BH2.1 and Compound BD2.1, the doping ratio of Compound BD2.1 was 4%, with a thickness of 20 nm;
The material of the hole barrier layer was Compound HB2.1, with a thickness of 10 nm;
The material of the electron transport layer was Compound ET2.1, with a thickness of 30 nm;
The material of the electron injection layer was Yb, with a thickness of 5 nm;
The material of the cathode was Mg:Ag (2:8) alloy, with a thickness of 12 nm;
The material of the cover layer was CP2.1, with a thickness of 70 nm.

In the Device Preparation and Test Example 2.4, according to the different hole-transport compounds used (which resulted in different materials for the hole injection layer and hole transport layer of the organic electroluminescent devices), the organic electroluminescent devices were divided into Reference Device B2.4A (the hole-transport compound was Compound HT1-Ref), Reference Device B2.4B (the hole-transport compound was Compound HT2-Ref), Test Device B2.4A (the hole-transport compound was Compound B58) and Test Device B2.4B (the hole-transport compound was Compound B224).

The driving voltage, luminous efficiency, and lifetime of Reference Device B2.4A, Reference Device 2.4B, Test Device 2.4A and Test Device B2.4B were tested. Results are shown in Table 2.8 and Table 2.9. In Table 2.8, the test data of each device were normalized based on reference device B2.4A. In Table 2.9, the test data of each device were normalized based on reference device B2.4B. Here, the lifetime refers to T95 lifetime, measured at room temperature with a current density of 15 mA/cm²; the efficiency refers to current efficiency, measured at a current density of 15 mA/cm².

TABLE 2.8

| Device | Voltage | Efficiency | Lifetime |
|---|---|---|---|
| Reference Device B2.4A | 100% | 100% | 100% |
| Test Device B2.4A | 100% | 131% | 120% |

TABLE 2.9

| Device | Voltage | Efficiency | Lifetime |
|---|---|---|---|
| Reference Device B2.4A | 100% | 100% | 100% |
| Test Device B2.4A | 99% | 116% | 107% |

Compared to Reference Device B2.4A, Test Device B2.4A exhibited higher efficiency and longer lifetime. This is because the hole transport layer material in Test Device B2.4A incorporates cyclopentylnaphthalene and diphenylfluorene groups, which improve the material mobility while maintaining relatively shallow energy levels. Additionally, the diphenylfluorene group has a large steric structure that can suppress aggregation and crystallization, thereby enhancing the film-forming properties of the material.

Compared to Reference Device B2.4B, Test Device B2.4B demonstrated higher efficiency and longer lifetime. This is attributed to the hole transport layer material in Test Device B2.4B, which reduces substitution at the 2- and 3-positions of cyclopentylnaphthalene (as simultaneous substitution at both positions would create a twisted structure). This modification enhances the conjugation of the compound, thereby improving charge carrier mobility and ultimately increasing the efficiency of the organic electroluminescent device.

### Device Preparation and Test Example 2.5

Multiple organic electroluminescent devices were fabricated using the following method, and their driving voltage, luminous efficiency, and lifetime were tested. The preparation process of the organic electroluminescent devices is as follows:

The pre-prepared ITO substrate was cleaned and dried, with the ITO substrate having an ITO layer serving as the anode, the thickness of the ITO layer was 100 nm; on the anode, a hole injection layer, a hole transport layer, an electron barrier layer (in some cases, also referred to as a light-emitting auxiliary layer), an organic light-emitting layer, a hole barrier layer, an electron transport layer, an electron injection layer, a cathode, and a cover layer were sequentially formed through evaporation processes in a stacked manner.

The material of the hole injection layer was Compound HT2.5 doped with Compound PD2.5, with a doping ratio of 2% for Compound PD2.5, and a thickness of 10 nm. The material of the hole transport layer was Compound HT2.5, with a thickness of 110 nm. The material of the hole barrier layer was Compound HB2.5, with a thickness of 5 nm. The material of the electron transport layer was a mixture of Compound ET2.5 and LiQ in a 1:1 ratio, with a thickness of 30 nm. The material of the electron injection layer was Yb, with a thickness of 5 nm. The material of the cathode was a magnesium-silver alloy, with a thickness of 10 nm. The material of the cover layer was Compound CP2.5, with a thickness of 70 nm.

In this embodiment, the differences in the organic electroluminescent devices lie in the electron barrier layer and organic light-emitting layer, while the materials and thicknesses of other layers remain the same. Based on the differences in the electron barrier layer and organic light-emitting layer, the organic electroluminescent devices include:
Reference device B2.5A (the material of the electron barrier layer was Compound EB2.5, with a thickness of 10 nm; the material of the organic light-emitting layer was a mixture of Compound BH2.5 and Compound BD2.5, with a doping ratio of 96:94, and a thickness of 20 nm);
Test device B2.5A (the material of the electron barrier layer was Compound B17, with a thickness of 10 nm; the material of the organic light-emitting layer was a mixture of Compound BH2.5 and Compound BD2.5, with a doping ratio of 96:94, and a thickness of 20 nm);
Reference device B2.5B (the material of the light-emitting auxiliary layer was Compound GP2.5, with a thickness of 40 nm; the material of the organic light-emitting layer was a mixture of Compounds GH2.5A, GH2.5B and GD2.5, with a doping ratio of 60:30:10, and a thickness of 32 nm);
Test device B2.5B (the material of the light-emitting auxiliary layer was Compound B41, with a thickness of 40 nm; the material of the organic light-emitting layer was a mixture of Compound GH2.5A, Compound GH2.5B and Compound GD2.5, with a doping ratio of 60:30:10, and a thickness of 32 nm);
Reference device B2.5C (the material of the light-emitting auxiliary layer was Compound RP2.5, with a thickness of 85 nm; the material of the organic light-emitting layer was a mixture of Compound RH2.5A, Compound RH2.5B and Compound RD2.5, with a doping ratio of 49:49:2, and a thickness of 45 nm);
Test device B2.5C (the material of the light-emitting auxiliary layer was Compound B267, with a thickness of 85 nm; the material of the organic light-emitting layer was a mixture of Compound RH2.5A, Compound RH2.5B and Compound RD2.5, with a doping ratio of 49:49:2, and a thickness of 45 nm).

In this Example 2.5, the structural formulas of the materials used are as follows:

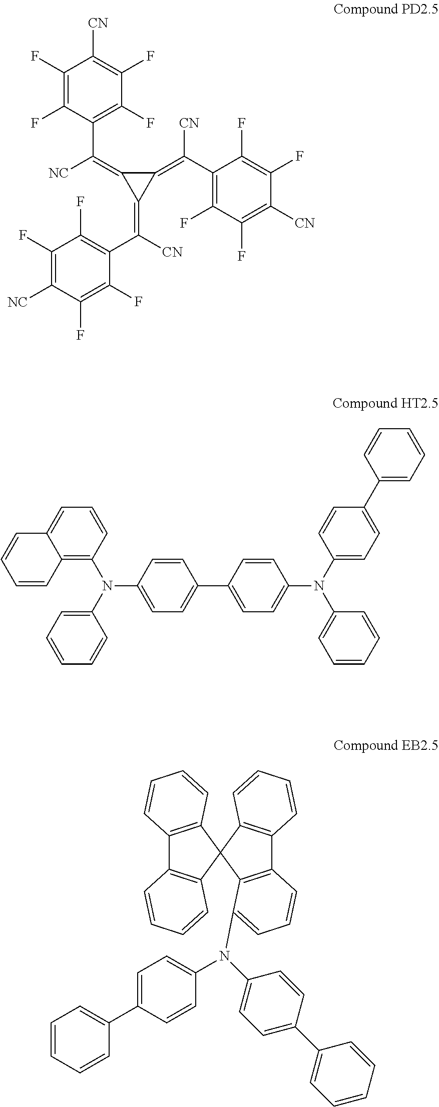 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F ; c1ccc(-c2ccc(N(c3ccccc3)c3ccc(-c4ccc(N(c5ccccc5)c5cccc6ccccc56)cc4)cc3)cc2)cc1 ; c1ccc(-c2ccc(N(c3ccc(-c4ccccc4)cc3)c3cccc4c3C3(c5ccccc5-c5ccccc53)c3ccccc3-4)cc2)cc1]

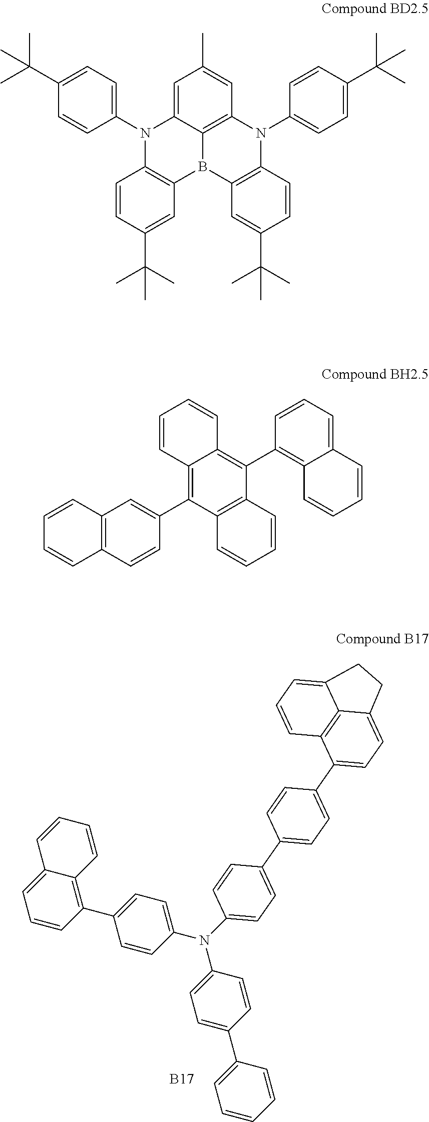

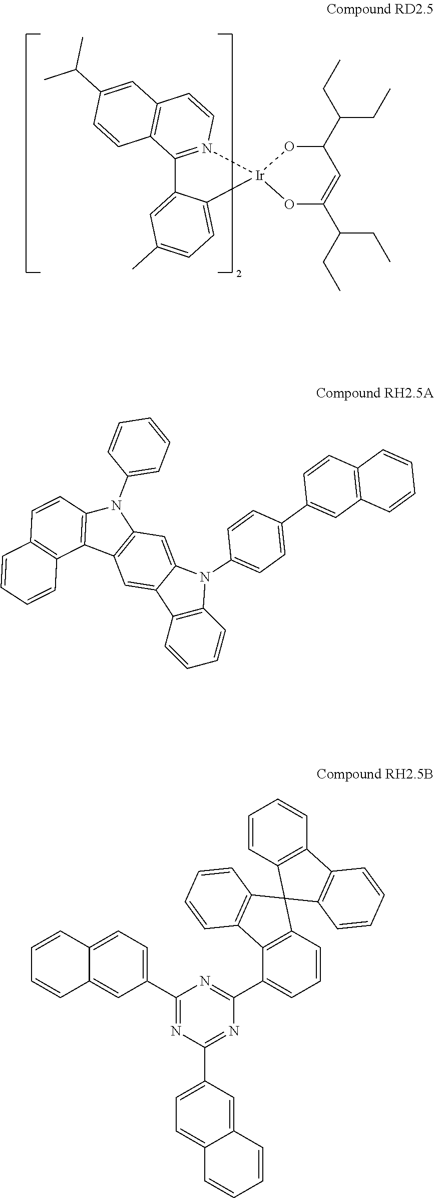

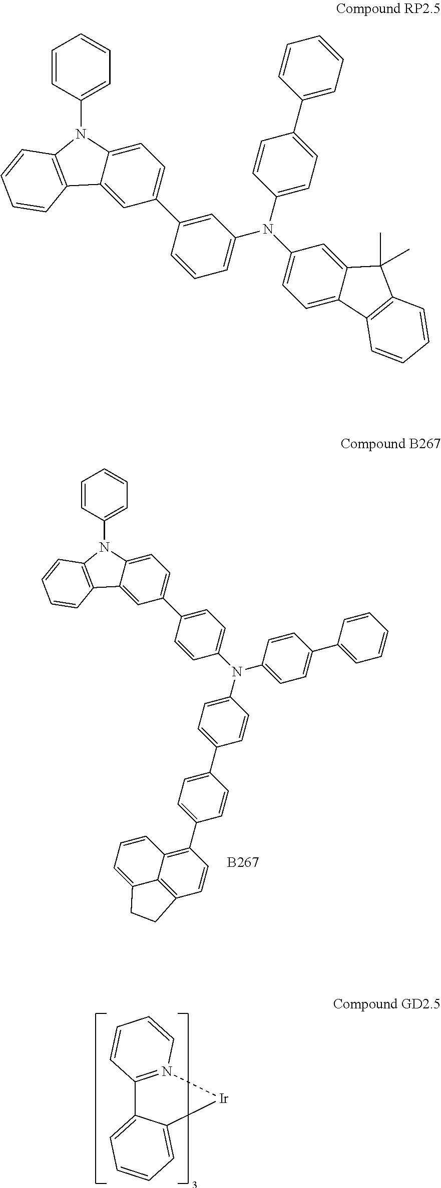

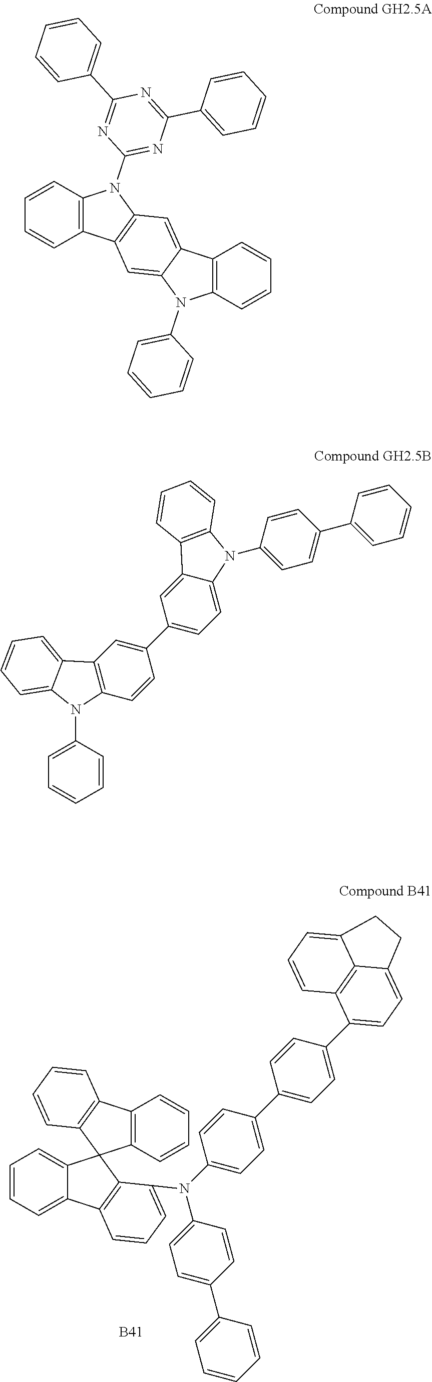

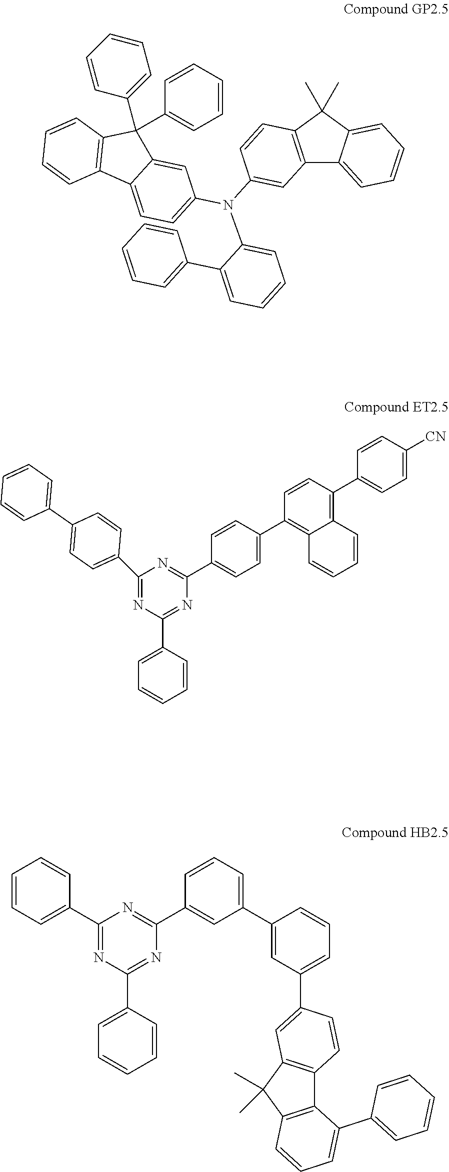 [SMILES: CC1(C)c2ccccc2-c2cc(N(c3ccc4c(c3)C(c3ccccc3)(c3ccccc3)c3ccccc3-4)c3ccccc3-c3ccccc3)ccc21 ; N#Cc1ccc(-c2ccc(-c3ccc(-c4nc(-c5ccccc5)nc(-c5ccc(-c6ccccc6)cc5)n4)cc3)c3ccccc23)cc1 ; CC1(C)c2cc(-c3cccc(-c4cccc(-c5nc(-c6ccccc6)nc(-c6ccccc6)n5)c4)c3)ccc2-c2c(-c3ccccc3)cccc21]

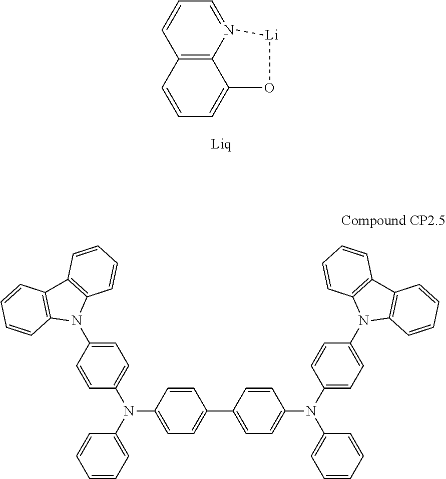

The driving voltage, luminous efficiency, and lifetime of Reference Device B2.5A, Test Device B2.5A, Reference Device B2.5B, Test Device B2.5B, Reference Device B2.5C, and Test Device B2.5C were tested. The test results are shown in Table 2.10, Table 2.11, and Table 2.12. In Table 2.10, the test data of each device were normalized based on reference device B2.5A. In Table 2.11, the test data of each device were normalized based on reference device B2.5B. In Table 2.12, the test data of each device were normalized based on reference device B2.5C. Here, the lifetime refers to T95 lifetime, measured at room temperature with a current density of 15 mA/cm²; the efficiency refers to current efficiency, measured at a current density of 15 mA/cm².

TABLE 2.10

| Device | Voltage | Efficiency |
|---|---|---|
| Reference Device B2.5A | 100% | 100% |
| Test Device B2.5A | 100% | 124% |

TABLE 2.11

| Device | Voltage | Efficiency |
|---|---|---|
| Reference Device B2.5B | 100% | 100% |
| Test Device B2.5B | 99% | 106% |

TABLE 2.12

| Device | Voltage | Efficiency |
|---|---|---|
| Reference Device B2.5C | 100% | 100% |
| Test Device B2.5C | 98% | 113% |

Compared to Reference Device B2.5A, Test Device B2.5A exhibited higher efficiency; compared to Reference Device B2.5B, Test Device B2.5B demonstrated higher efficiency; and compared to Reference Device B2.5C, Test Device B2.5C showed higher efficiency. This difference in efficiency is primarily attributed to the materials used in the electron barrier layer/light-emitting auxiliary layer of the reference devices, which incorporate a cyclopentylnaphthalene structure. This structural feature enhances hole mobility and improves energy level alignment between the electron barrier layer/light-emitting auxiliary layer and the organic light-emitting layer, thereby increasing efficiency and contributing to the reduction in driving voltage.

### Synthesis Example 3.1

### Synthesis of Compound C23

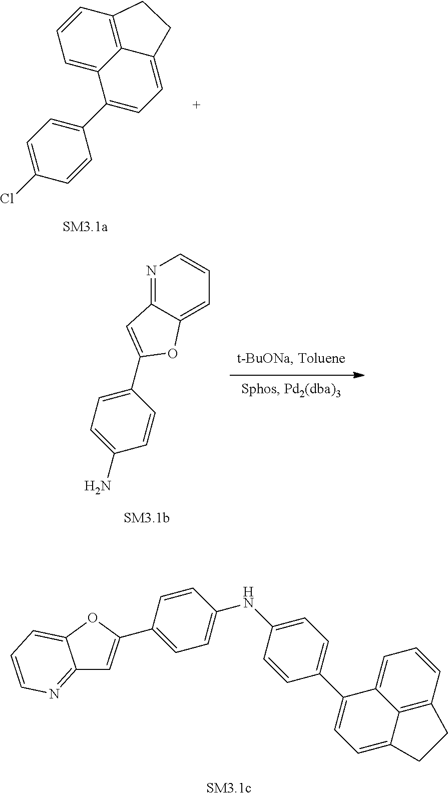 [SMILES: Clc1ccc(-c2ccc3c4c(cccc24)CC3)cc1 ; Nc1ccc(-c2cc3ncccc3o2)cc1 ; c1cnc2cc(-c3ccc(Nc4ccc(-c5ccc6c7c(cccc57)CC6)cc4)cc3)oc2c1]

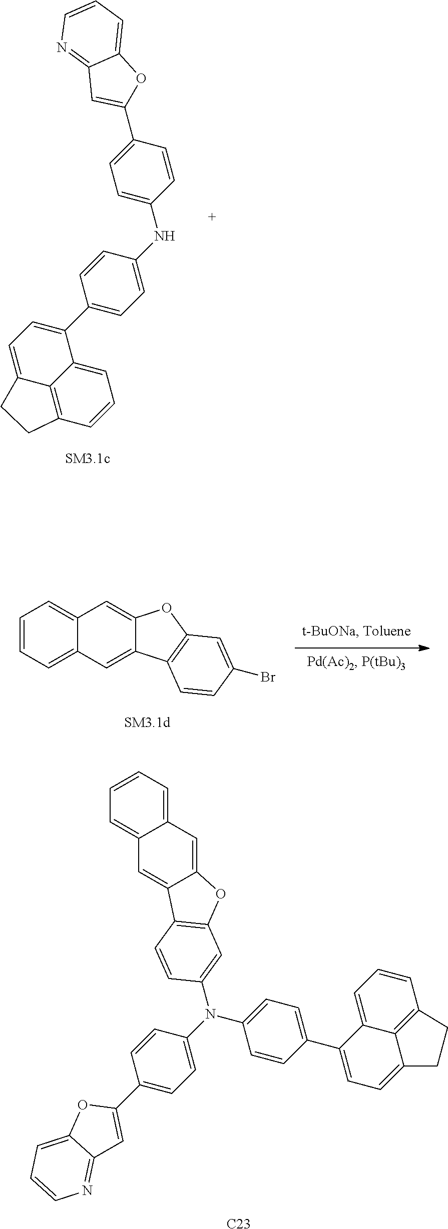 [SMILES: c1cnc2cc(-c3ccc(Nc4ccc(-c5ccc6c7c(cccc57)CC6)cc4)cc3)oc2c1 ; Brc1ccc2c(c1)oc1cc3ccccc3cc12 ; c1ccc2cc3c(cc2c1)oc1cc(N(c2ccc(-c4cc5ncccc5o4)cc2)c2ccc(-c4ccc5c6c(cccc46)CC5)cc2)ccc13 ; "C23"]

Toluene solvent (150 mL) was added to a reaction flask, followed by the addition of Intermediate SM3.1a (5.58 g, 21 mmol), Intermediate SM3.1b (4.20 g, 20 mmol), and sodium tert-butoxide (2.88 g, 30 mmol). The mixture was purged with nitrogen, after which Pd₂(dba)₃ (0.183 g, 0.2 mmol) and Sphos (0.164 g, 0.4 mmol) were added. The reaction was refluxed under nitrogen for 2 hours. The resulting mixture was subjected to suction filtration and washed with methanol to afford recrystallized solid, yielding Intermediate SM3.1c.

Toluene solvent was added to the flask, followed by the sequential addition of Intermediate SM3.1c (6.43 g, 15 mmol), Intermediate SM3.1d (4.72 g, 16 mmol), and sodium tert-butoxide (2.16 g, 22.5 mmol). The system was purged with nitrogen, and palladium acetate (0.034 g, 0.15 mmol) and tri-tert-butylphosphine (0.061 g, 0.3 mmol) were added. The nitrogen purging process was repeated, and the mixture was refluxed for 2 hours. Upon completion of the reaction, the reaction was cooled to room temperature and filtered through Celite to obtain the filtrate. The filtrate was concentrated, heated, and treated with a small amount of ethanol, then allowed to stand at room temperature for recrystallization. Suction filtration and washing with ethanol afforded recrystallized solid, yielding Compound C23 as a pale yellow solid. The purity was determined to be 99.57%, with a yield of 88%. MS: 654.77; C₄₇H₃₀N₂O₂.

Following the synthetic procedure of Compound C23, Starting Material 3.1 was used in place of Intermediate SM3.1a, Starting Material 3.2 replaced Intermediate SM3.1b, and Starting Material 3.3 was used in place of Intermediate SM3.1d to synthesize Compound C4, Compound C9, Compound C4, Compound C14, Compound C15, Compound C28, Compound C22, Compound C30, Compound C43, Compound C58, and Compound C60. The Compounds and their corresponding Starting Materials are listed in Table 3.0.

TABLE 3.0

| Compound | | Starting Material 3.1 |
|---|---|---|
| 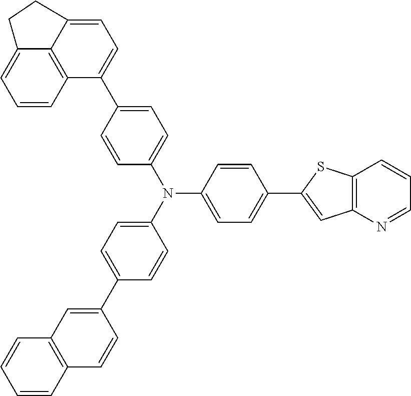 [SMILES: c1ccc2cc(-c3ccc(N(c4ccc(-c5cc6ncccc6s5)cc4)c4ccc(-c5ccc6c7c(cccc57)CC6)cc4)cc3)ccc2c1] | | 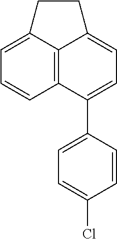 [SMILES: Clc1ccc(-c2ccc3c4c(cccc24)CC3)cc1] |
| C4 | | |
| 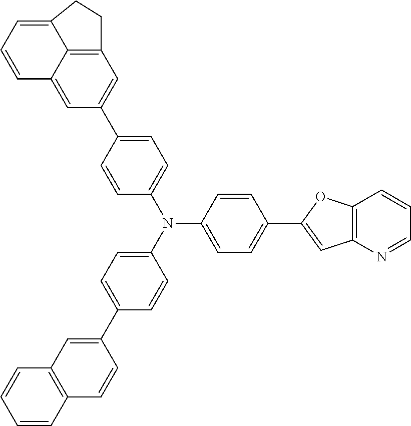 [SMILES: c1ccc2cc(-c3ccc(N(c4ccc(-c5cc6c7c(cccc7c5)CC6)cc4)c4ccc(-c5cc6ncccc6o5)cc4)cc3)ccc2c1] | | 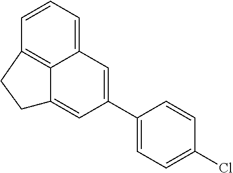 [SMILES: Clc1ccc(-c2cc3c4c(cccc4c2)CC3)cc1] |
| C9 | | |
| 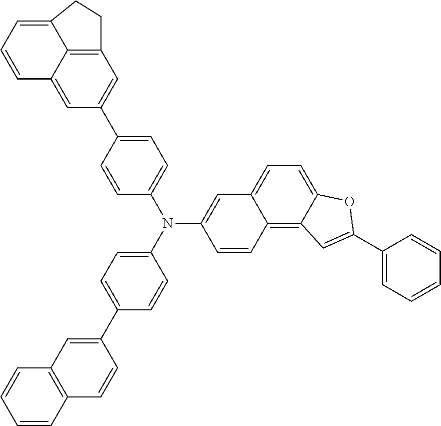 [SMILES: c1ccc(-c2cc3c(ccc4cc(N(c5ccc(-c6ccc7ccccc7c6)cc5)c5ccc(-c6cc7c8c(cccc8c6)CC7)cc5)ccc43)o2)cc1] | | 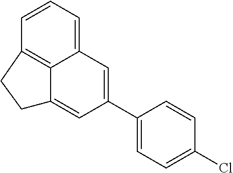 [SMILES: Clc1ccc(-c2cc3c4c(cccc4c2)CC3)cc1] |
| C14 | | |
| 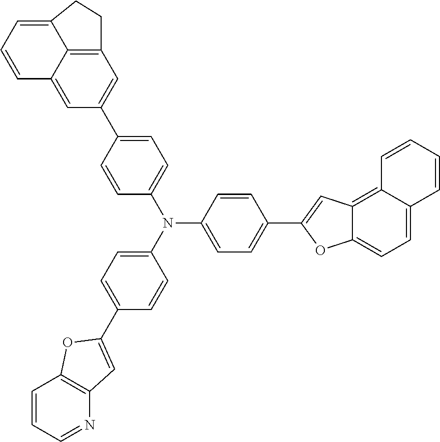 [SMILES: c1cc2c3c(cc(-c4ccc(N(c5ccc(-c6cc7ncccc7o6)cc5)c5ccc(-c6cc7c(ccc8ccccc87)o6)cc5)cc4)cc3c1)CC2] | | 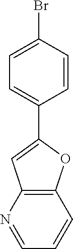 [SMILES: Brc1ccc(-c2cc3ncccc3o2)cc1] |
| C15 | | |
| 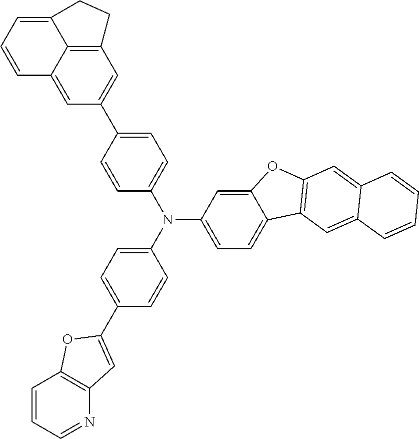 [SMILES: c1ccc2cc3c(cc2c1)oc1cc(N(c2ccc(-c4cc5c6c(cccc6c4)CC5)cc2)c2ccc(-c4cc5ncccc5o4)cc2)ccc13] | | 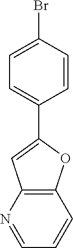 [SMILES: Brc1ccc(-c2cc3ncccc3o2)cc1] |
| C28 | | |
| 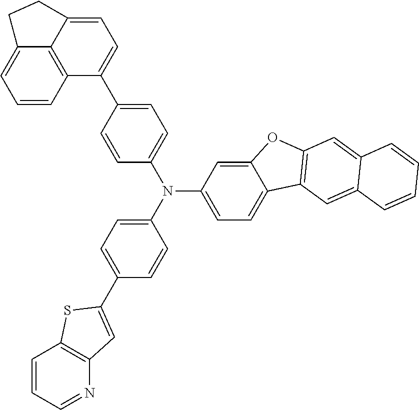 [SMILES: c1ccc2cc3c(cc2c1)oc1cc(N(c2ccc(-c4cc5ncccc5s4)cc2)c2ccc(-c4ccc5c6c(cccc46)CC5)cc2)ccc13] | | 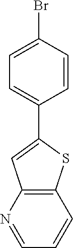 [SMILES: Brc1ccc(-c2cc3ncccc3s2)cc1] |
| C22 | | |
| 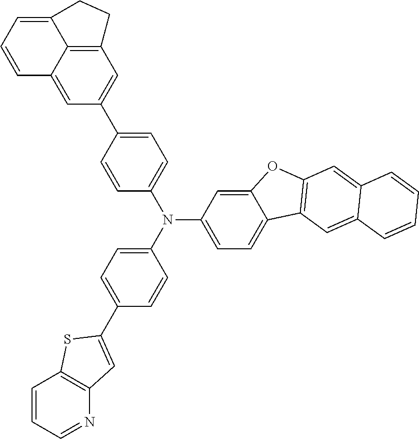 [SMILES: c1ccc2cc3c(cc2c1)oc1cc(N(c2ccc(-c4cc5c6c(cccc6c4)CC5)cc2)c2ccc(-c4cc5ncccc5s4)cc2)ccc13] | | 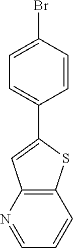 [SMILES: Brc1ccc(-c2cc3ncccc3s2)cc1] |
| C30 | | |
| 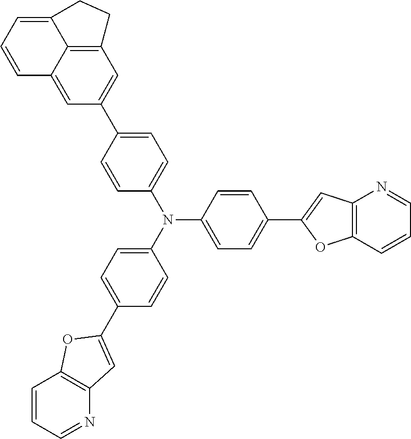 [SMILES: c1cc2c3c(cc(-c4ccc(N(c5ccc(-c6cc7ncccc7o6)cc5)c5ccc(-c6cc7ncccc7o6)cc5)cc4)cc3c1)CC2] | | 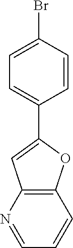 [SMILES: Brc1ccc(-c2cc3ncccc3o2)cc1] |
| C43 | | |
| 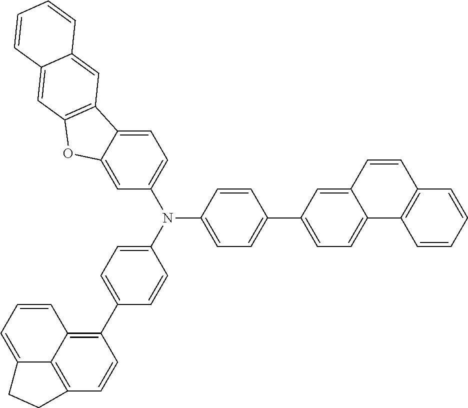 [SMILES: c1ccc2cc3c(cc2c1)oc1cc(N(c2ccc(-c4ccc5c(ccc6ccccc65)c4)cc2)c2ccc(-c4ccc5c6c(cccc46)CC5)cc2)ccc13] | | 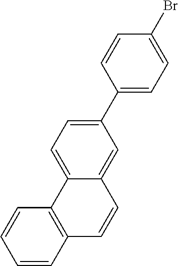 [SMILES: Brc1ccc(-c2ccc3c(ccc4ccccc43)c2)cc1] |
| C58 | | |
| 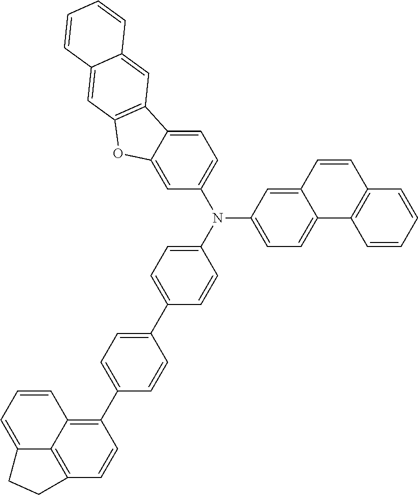 [SMILES: c1ccc2cc3c(cc2c1)oc1cc(N(c2ccc(-c4ccc(-c5ccc6c7c(cccc57)CC6)cc4)cc2)c2ccc4c(ccc5ccccc54)c2)ccc13] | | 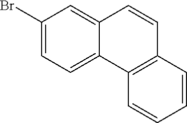 [SMILES: Brc1ccc2c(ccc3ccccc32)c1] |
| C60 | | |
| | Starting Material 3.2 | Starting Material 3.3 |
| | 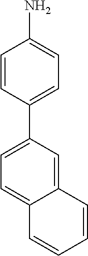 [SMILES: Nc1ccc(-c2ccc3ccccc3c2)cc1] | 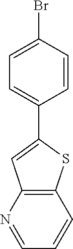 [SMILES: Brc1ccc(-c2cc3ncccc3s2)cc1] |
| | 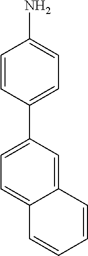 [SMILES: Nc1ccc(-c2ccc3ccccc3c2)cc1] | 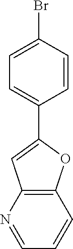 [SMILES: Brc1ccc(-c2cc3ncccc3o2)cc1] |
| | 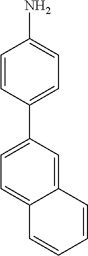 [SMILES: Nc1ccc(-c2ccc3ccccc3c2)cc1] | 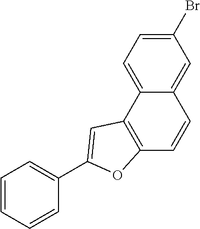 [SMILES: Brc1ccc2c(ccc3oc(-c4ccccc4)cc32)c1] |
| | 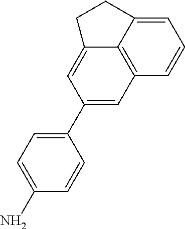 [SMILES: Nc1ccc(-c2cc3c4c(cccc4c2)CC3)cc1] | 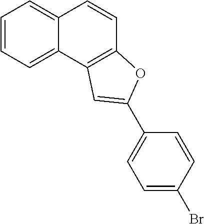 [SMILES: Brc1ccc(-c2cc3c(ccc4ccccc43)o2)cc1] |
| | 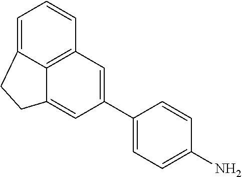 [SMILES: Nc1ccc(-c2cc3c4c(cccc4c2)CC3)cc1] | 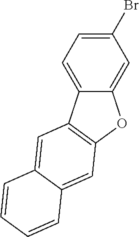 [SMILES: Brc1ccc2c(c1)oc1cc3ccccc3cc12] |
| | 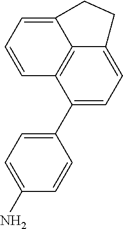 [SMILES: Nc1ccc(-c2ccc3c4c(cccc24)CC3)cc1] | 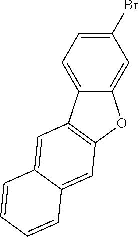 [SMILES: Brc1ccc2c(c1)oc1cc3ccccc3cc12] |
| | 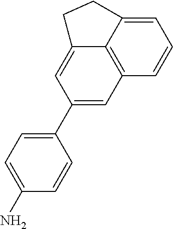 [SMILES: Nc1ccc(-c2cc3c4c(cccc4c2)CC3)cc1] | 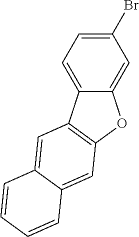 [SMILES: Brc1ccc2c(c1)oc1cc3ccccc3cc12] |
| | 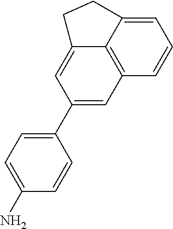 [SMILES: Nc1ccc(-c2cc3c4c(cccc4c2)CC3)cc1] | 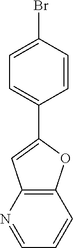 [SMILES: Brc1ccc(-c2cc3ncccc3o2)cc1] |
| | 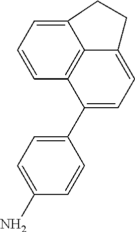 [SMILES: Nc1ccc(-c2ccc3c4c(cccc24)CC3)cc1] | 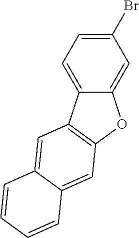 [SMILES: Brc1ccc2c(c1)oc1cc3ccccc3cc12] |
| | 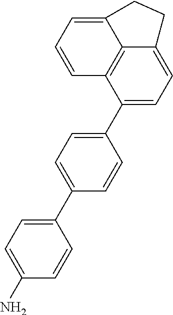 [SMILES: Nc1ccc(-c2ccc(-c3ccc4c5c(cccc35)CC4)cc2)cc1] | 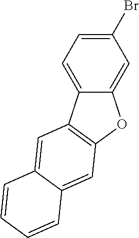 [SMILES: Brc1ccc2c(c1)oc1cc3ccccc3cc12] |

### Refractive Index Testing

The refractive indices of Compound C4, Compound C9, Compound C14, Compound C15, Compound C23, Compound C28, Compound C22, Compound C30, Compound C43, Compound C58, Compound C60, as well as the Reference Compound CP3.1 and Reference Compound CP3.2, were measured. The test results are shown in Table 3.1.

For the refractive index tests, an ellipsometer was used with a scanning range of 245~1000 nm. The test was conducted on thin films deposited on silicon wafers, with a film thickness of 50 nm.

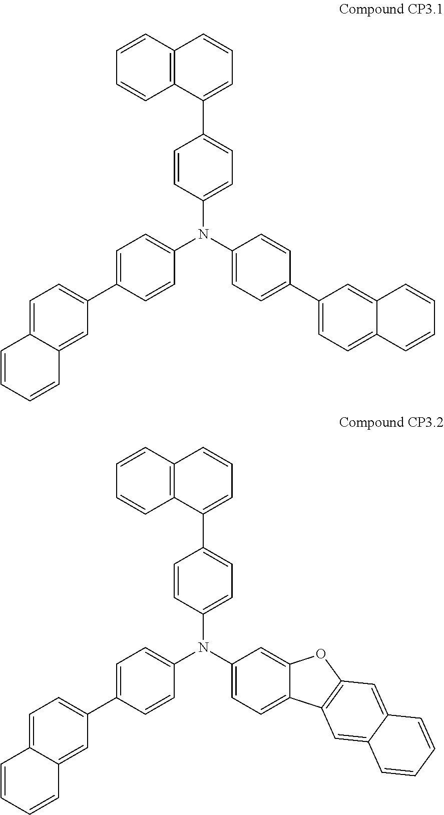 [SMILES: c1ccc2cc(-c3ccc(N(c4ccc(-c5ccc6ccccc6c5)cc4)c4ccc(-c5cccc6ccccc56)cc4)cc3)ccc2c1 ; c1ccc2cc(-c3ccc(N(c4ccc(-c5cccc6ccccc56)cc4)c4ccc5c(c4)oc4cc6ccccc6cc45)cc3)ccc2c1]

TABLE 3.1

| | Refractive Index | | |
|---|---|---|---|
| Compound | 460 nm | 530 nm | 620 nm |
| C4 | 2.218 | 2.085 | 2.007 |
| C9 | 2.280 | 2.140 | 2.059 |
| C14 | 2.322 | 2.160 | 2.068 |
| C15 | 2.259 | 2.120 | 2.039 |
| C23 | 2.267 | 2.125 | 2.043 |
| C28 | 2.310 | 2.145 | 2.053 |
| C22 | 2.263 | 2.119 | 2.036 |
| C30 | 2.303 | 2.127 | 2.049 |
| C43 | 2.231 | 2.091 | 2.016 |
| C58 | 2.317 | 2.191 | 2.104 |
| C60 | 2.315 | 2.190 | 2.104 |
| Reference CP3.1 | 1.98 | 1.85 | 1.77 |
| Reference CP3.2 | 2.200 | 2.054 | 1.986 |

According to Table 3.1, it can be observed that the compounds in some of the embodiments of the present disclosure exhibit high refractive indices. On one hand, the compounds of the present disclosure can enhance molecular polarizability by introducing heteroatoms such as oxygen or nitrogen. On the other hand, when a heterocyclic ring is connected to a benzene ring (or other aromatic rings), the structure remains nearly planar, with the dihedral angle between the heterocyclic plane and the adjacent aromatic plane close to 0°, approaching a coplanar conformation. This increases the conjugation of the molecule, which is beneficial for improving the refractive index. Moreover, the enhanced conjugation of these compounds also leads to stronger UV absorption, which helps protect other functional materials in organic electroluminescent devices. Additionally, the compounds of the present disclosure feature a cycloalkylnaphthalene structure, which not only maintains a high refractive index but also moderately increases the three-dimensional steric hindrance of the molecule. This structural feature helps improve the thermal stability of the material.

### Absorption Intensity Test and Glass Transition Temperature Test

Absorption Intensity Test: For Compound C4, Compound C9, Compound C14, Compound C15, Compound C23, Compound C28, Compound C22, Compound C30, Compound C43, Compound C58, Compound C60, as well as the Reference Compound CP3.1 and Reference Compound CP3.2, 50 nm thin films were prepared. The absorption intensity was measured using a UV spectrophotometer, and the test results at wavelengths of 400 nm and 450 nm are summarized in Table 3.2.

Glass Transition Temperature Test: The glass transition temperatures of Compound C4, Compound C9, Compound C14, Compound C15, Compound C23, Compound C28, Compound C22, Compound C30, Compound C43, Compound C58, Compound C60, as well as the Reference Compound CP3.1 and Reference Compound CP3.2, were measured using DSC (differential scanning calorimetry) (selecting the second heating cycle, with the temperature range from room temperature to 300° C.). The test results are shown in Table 3.2.

TABLE 3.2

| | Absorption Intensity | | Glass Transition |
|---|---|---|---|
| Compound | 400 nm | 450 nm | Temperature Tg (° C.) |
| C4 | 0.84 | 0 | 137 |
| C9 | 0.82 | 0 | 131 |
| C14 | 0.89 | 0 | 127 |
| C15 | 0.78 | 0 | 122 |
| C23 | 0.92 | 0 | 144 |
| C28 | 0.94 | 0 | 122 |
| C22 | 0.91 | 0 | 132 |
| C30 | 0.94 | 0 | 134 |
| C43 | 0.87 | 0 | 129 |
| C58 | 0.90 | 0 | 132 |
| C60 | 0.90 | 0 | 135 |
| Reference CP3.1 | 0.52 | 0 | 129 |
| Reference CP3.2 | 0.73 | 0 | 121 |

According to Table 3.2, it can be observed that some of the compounds provided in the embodiments of the present disclosure exhibit strong absorption intensity at 400 nm. When these compounds are used as cover layers in organic electroluminescent devices, they can absorb ultraviolet light from the external environment, thereby protecting the organic electroluminescent devices from damage caused by ambient UV radiation and slowing down the aging process of the devices, which helps extend their lifetime. At the same time, these compounds exhibit minimal absorption at 450 nm and longer wavelengths, thus avoiding absorption of the device's own emitted light and preventing any negative impact on the light extraction efficiency of the organic electroluminescent devices.

According to Table 3.2, it can also be seen that some of the compounds provided in the embodiments of the present disclosure possess high glass transition temperatures. On one hand, this enhances the stability of these compounds during the evaporation process; on the other hand, it prevents the formed thin films from undergoing vitrification, thereby helping to maintain the stability of the deposited layers. As a result, these compounds exhibit excellent film-forming properties.

### Device Preparation and Test Example 3.1

An organic electroluminescent device was fabricated by the following method: A pre-prepared ITO substrate with an ITO thickness of 100 nm was cleaned and dried. On the anode (ITO), a hole injection material and a hole-transport material were sequentially co-deposited to form a hole injection layer and a hole transport layer (HTL), followed by an electron barrier layer (EBL) material. Subsequently, an organic light-emitting layer was deposited by co-evaporating BH and BD materials. On the organic light-emitting layer, a hole barrier layer (HBL) material, an electron transport layer (ETL) material, and an electron injection layer (EIL) material were deposited. Then, a cathode (Mg—Ag alloy) was evaporated. Above the cathode, a cover layer (CPL) material was deposited with a thickness of 65 nm.

The material of the hole injection layer was a mixture of Compound PD2.5 and Compound HT2.5, with a doping ratio of Compound PD2.5 of 2%, and a thickness of 10 nm.

The material of the hole transport layer was Compound HT2.5, with a thickness of 110 nm.

The material of the electron barrier layer was Compound EB2.5, with a thickness of 5 nm.

The material of the organic light-emitting layer was a mixture of Compound BH2.5 and Compound BD2.5, with a doping ratio of Compound BD2.5 of 3%, and a thickness of 20 nm.

The material of the hole barrier layer was Compound HB2.5, with a thickness of 5 nm.

The material of the electron transport layer was a 1:1 mixture of Compound ET2.5 and LiQ, with a thickness of 30 nm.

The material of the electron injection layer was metal Yb, with a thickness of 1 nm.

The material of the cathode was an Mg—Ag alloy, with a thickness of 13 nm.

In this example, multiple different organic electroluminescent devices were fabricated using different cover layer materials. The only difference among these devices lies in the cover layer materials, while all other layers maintain identical materials and thicknesses. The organic electroluminescent devices prepared in this example include:
Test Device C4 (cover layer material was Compound C4)
Test Device C14 (cover layer material was Compound C14)
Test Device C22 (cover layer material was Compound C22)
Test Device C23 (cover layer material was Compound C23)
Test Device C28 (cover layer material was Compound C28)
Test Device C43 (cover layer material was Compound C43)
Test Device C58 (cover layer material was Compound C58)
Reference Device C3.1 (cover layer material was Compound CP3.1)
Test Device C3.2 (cover layer material was Compound CP3.2)

The driving voltage, current efficiency, and lifetime of Test Devices C4, C14, C22, C23, C28, C43, C58, Reference Device C3.1, and Test Device C3.2 were measured under a current density of 10 mA/cm². In Table 3.3, all test data were normalized using the results of Reference Device C3.1 as the baseline.

TABLE 3.3

| Device | Voltage (V) | Efficiency (cd/A) | Lifetime (h) |
|---|---|---|---|
| Test Device C4 | 99% | 103% | 103% |
| Test Device C14 | 98% | 104% | 105% |
| Test Device C22 | 99% | 106% | 101% |
| Test Device C23 | 98% | 106% | 102% |
| Test Device C28 | 98% | 107% | 101% |
| Test Device C43 | 99% | 104% | 100% |
| Test Device C58 | 99% | 107% | 100% |
| Ref. Device C3.1 | 100% | 100% | 100% |
| Ref. Device C3.2 | 100% | 103% | 100% |

Other embodiments of the present disclosure will be apparent to those skilled in the art from consideration of the specification and practice of the present disclosure disclosed herein. The present application is intended to cover any variations, uses, or adaptations of the present disclosure, which are in accordance with the general principles of the present disclosure and include common general knowledge or conventional technical means in the art that are not disclosed in the present disclosure. The specification and embodiments are illustrative, and the real scope and spirit of the present disclosure is defined by the appended claims.

1. An aromatic amine compound, wherein a structural formula of the aromatic amine, compound represented by Chemical Formula A or Chemical Formula B: 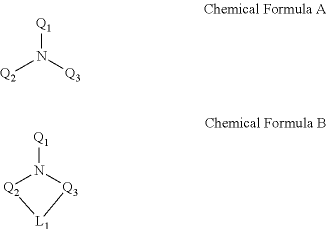
wherein L₁ is selected from a single bond, a substituted or unsubstituted alkyl group with 1~10 carbon atoms, a substituted or unsubstituted cycloalkyl group with 1~10 carbon atoms, a substituted or unsubstituted aryl group with 6~30 carbon atoms, or a substituted or unsubstituted heteroaryl group with 3~30 carbon atoms;
Q₁, Q₂, and Q₃ are each independently selected from a substituted or unsubstituted aryl group with 6~50 carbon atoms or a substituted or unsubstituted heteroaryl group with 3~50 carbon atoms, and at least one of Q₁, Q₂, or Q₃ contains at least one group represented by Chemical Formula P1 to Chemical Formula P6: 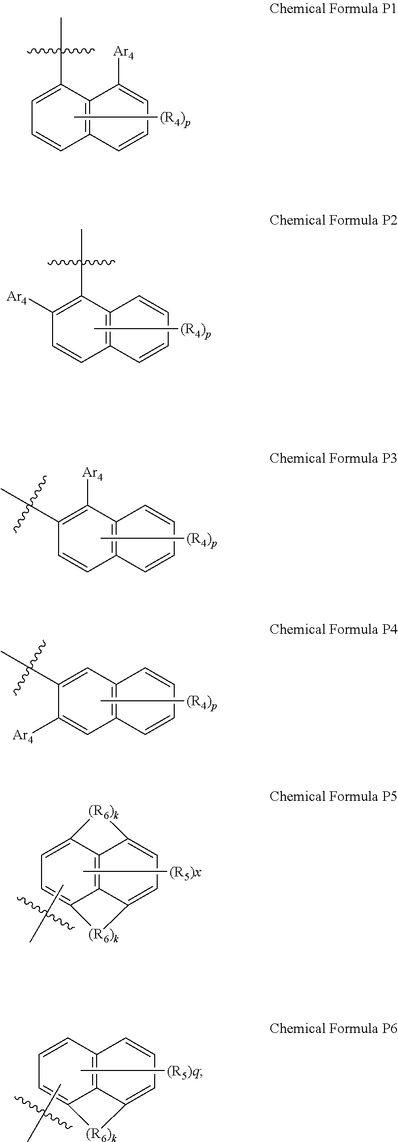
wherein Ar₄ is selected from hydrogen, deuterium, a substituted or unsubstituted alkyl group with 1~210 carbon atoms, a substituted or unsubstituted cycloalkyl group with 3~10 carbon atoms, a substituted or unsubstituted aryl group with 6~30 carbon atoms, a substituted or unsubstituted heteroaryl group with 3~30 carbon atoms, or a substituted or unsubstituted cycloalkane-naphthyl group with 14~30 carbon atoms,
R₄ and R₅ are the same or different, each independently selected from deuterium, a substituted or unsubstituted alkyl group with 1~20 carbon atoms, a substituted or unsubstituted cycloalkyl group with 3~10 carbon atoms, a substituted or unsubstituted aryl group with 6~20 carbon atoms, a substituted or unsubstituted heteroaryl group with 3~20 carbon atoms, a substituted or unsubstituted silyl group, or a substituted or unsubstituted arylsilyl group with 6~20 carbon atoms;
R₆ is C(R₇R₈); R₇ and R₈ are each independently selected from hydrogen, deuterium, fluorine, an alkyl group with 1~4 carbon atoms, or a substituted or unsubstituted aryl group with 6~12 carbon atoms;
p represents an integer from 0 to 6; when p is greater than 1, any two R₄ groups are the same or different;
q represents an integer from 0 to 4; when q is greater than 1, any two R₅ groups are the same or different;
x represents an integer from 0 to 3; when x is greater than 1, any two R₅ groups are the same or different;
k is an integer from 2 to 4; any two R₇ groups are the same or different; any two R₈ groups are the same or different;
when L₁, Q₁, Q₁, Q₂, Q₃, R₄, R₅, R₇, R₈, and Ar₄ have substituents, these substituents are the same or different and are each independently selected from deuterium, halogen, an alkylsilyl group with 1~30 carbon atoms, an arylsilyl group with 6~30 carbon atoms, an arylamino group with 6~-30 carbon atoms, an alkyl group with 1~30 carbon atoms, a cycloalkyl group with 5~10 carbon atoms, an alkenyl group with 2~30 carbon atoms, an aryl group with 6~~30 carbon atoms, or a heteroaryl group with 3~30 carbon atoms;
— represents a connecting bond.

2. The aromatic amine compound according to claim 1, wherein R₆ is CH₂ or CD₂, and k is 2.

3. The aromatic amine compound according to claim 1, wherein a structural formula of the aromatic amine compound represented by Chemical Formula I: 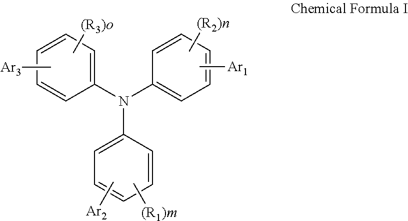
wherein Ar₃ is selected from any one of the following groups: 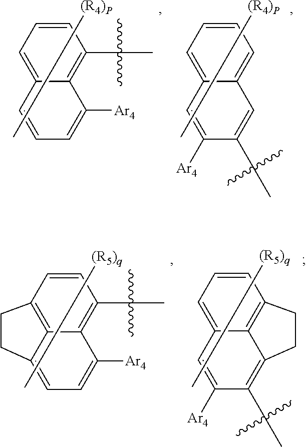
Ar₁ and Ar₂ are the same or different and each independently represent hydrogen, deuterium, a substituted or unsubstituted alkyl group having 1 to 10 carbon atoms, a substituted or unsubstituted cycloalkyl group having 1 to 10 carbon atoms, a substituted or unsubstituted aryl group having 6 to 30 carbon atoms, a substituted or unsubstituted heteroaryl group having 3 to 30 carbon atoms, or a substituted or unsubstituted cycloalkylnaphthalene structure. The heteroaryl group contains at least one heteroatom selected from O, S, N, or Si;
R₁-R₃ are the same or different and each independently represent deuterium, a substituted or unsubstituted alkyl group having 1 to 20 carbon atoms, a substituted or unsubstituted cycloalkyl group having 3 to 10 carbon atoms, a substituted or unsubstituted silyl group, or a substituted or unsubstituted arylsilyl group having 6 to 20 carbon atoms;
when substituents are present on Ar₁, Ar₂, Ar₄, or R₁-R₅, the substituents are the same or different and are each independently selected from deuterium, halogen, an alkylsilyl group having 1 to 30 carbon atoms, an arylsilyl group having 6 to 30 carbon atoms, an arylamino group having 6 to 30 carbon atoms, an alkyl group having 1 to 30 carbon atoms, an alkenyl group having 2 to 30 carbon atoms, or an aryl group having 6 to 30 carbon atoms;
when Ar₃ is selected as 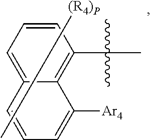
at least one benzene ring directly bonded to the naphthyl group has all hydrogens substituted with deuterium;
m represents an integer from 0 to 5;
n represents an integer from 0 to 5;
o represents an integer from 0 to 4.

4. The aromatic amine compound according to claim 3, wherein the aryl group is selected from any one of methylfluorenyl, a cycloalkylfluorenyl group having 5 to 10 carbon atoms, 9,9-diphenylfluorenyl, or spirobifluorenyl.

5. The aromatic amine compound according to claim 3, wherein the cycloalkylnaphthalene is selected from one of the following structures:

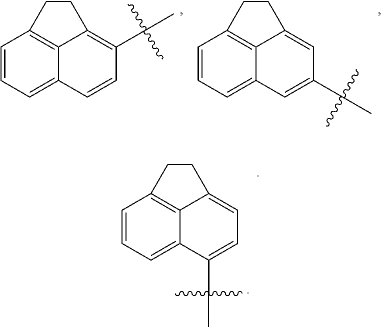 [SMILES: *c1ccc2cccc3c2c1CC3 ; *c1cc2c3c(cccc3c1)CC2 ; *c1ccc2c3c(cccc13)CC2]

6. The aromatic amine compound according to claim 3, wherein Ar₁, Ar₂, and Ar₄ are each independently selected from a substituted or unsubstituted structure represented by one of formulas A-1 to A-34:

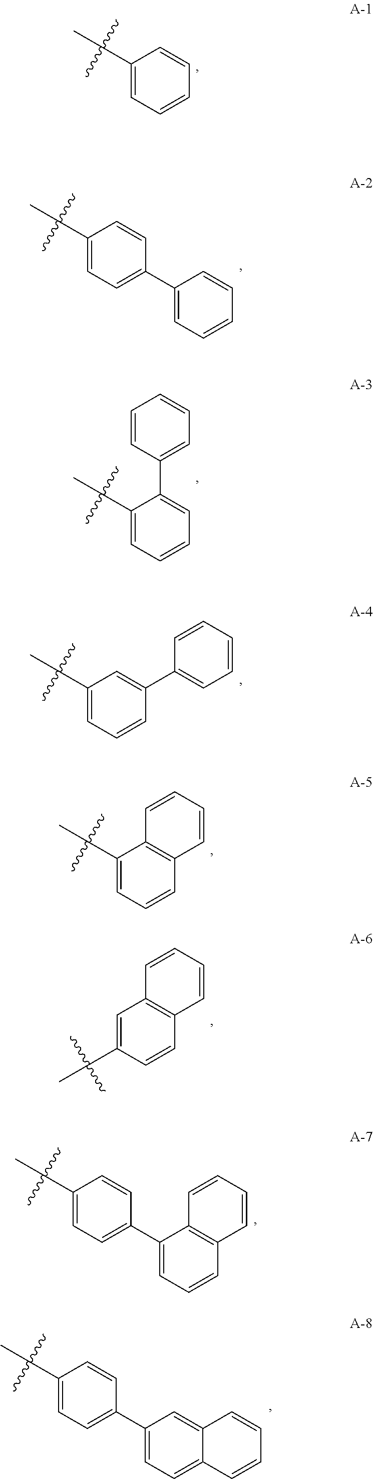

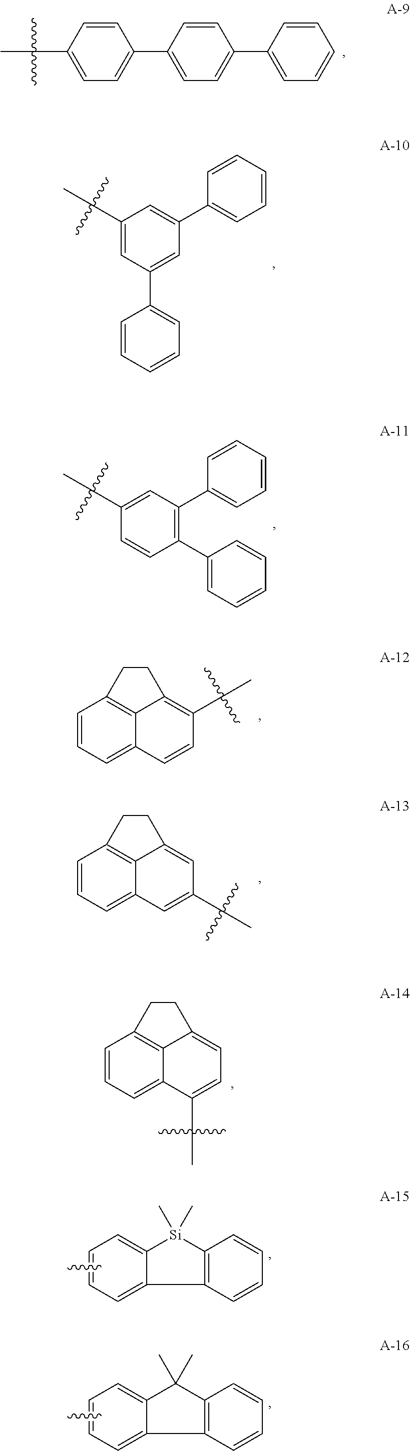

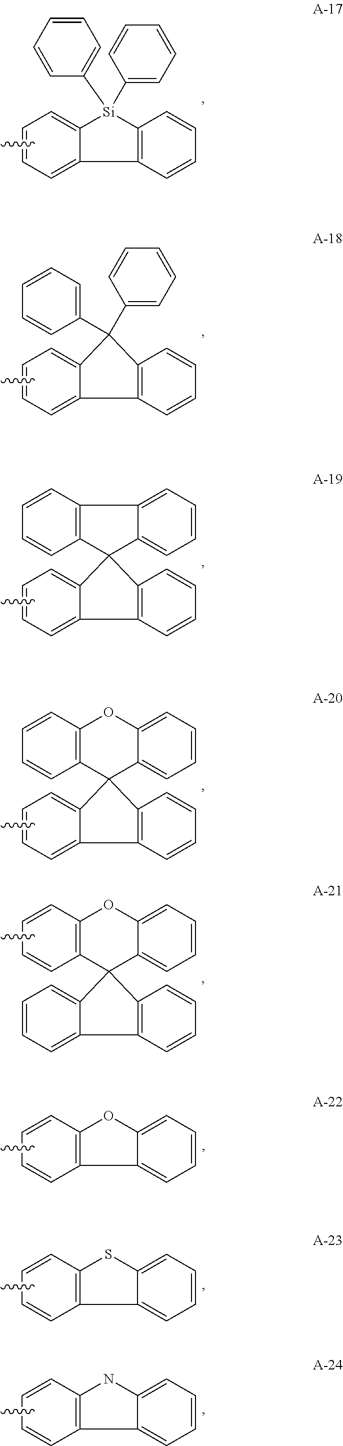

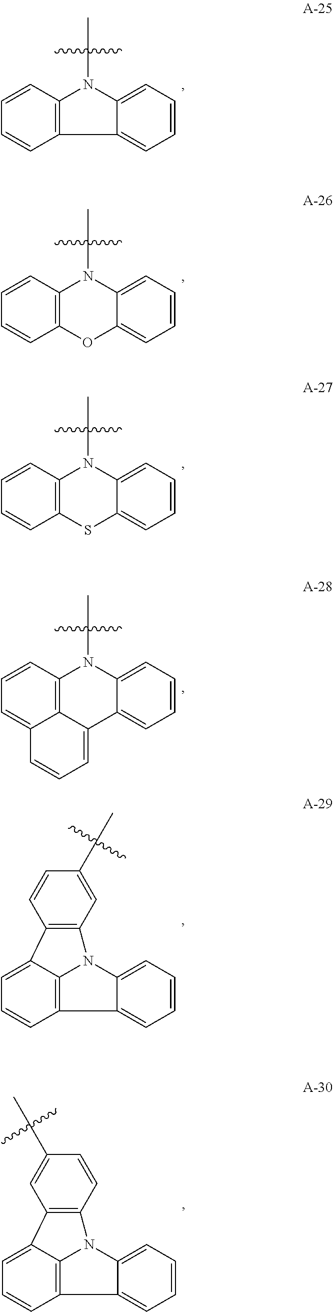 [SMILES: *n1c2ccccc2c2ccccc21 ; *N1c2ccccc2Oc2ccccc21 ; *N1c2ccccc2Sc2ccccc21 ; *N1c2ccccc2-c2cccc3cccc1c23 ; *c1ccc2c3cccc4c5ccccc5n(c2c1)c43 ; *c1ccc2c(c1)c1cccc3c4ccccc4n2c31]

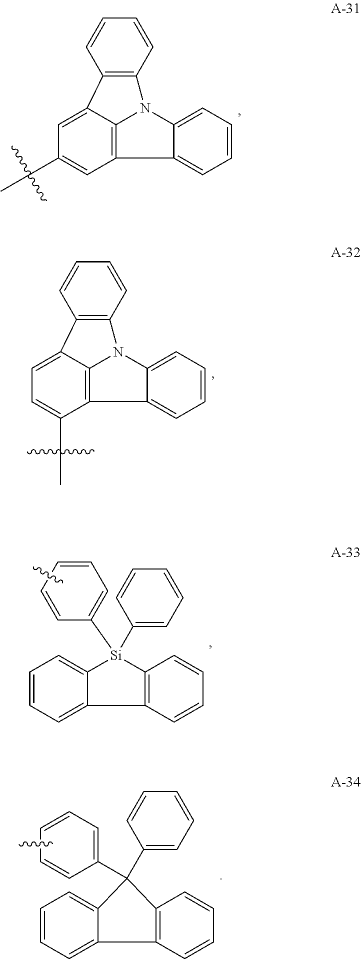

7. The aromatic amine compound according to claim 3, wherein the aromatic amine compound is selected from one of the following structures:

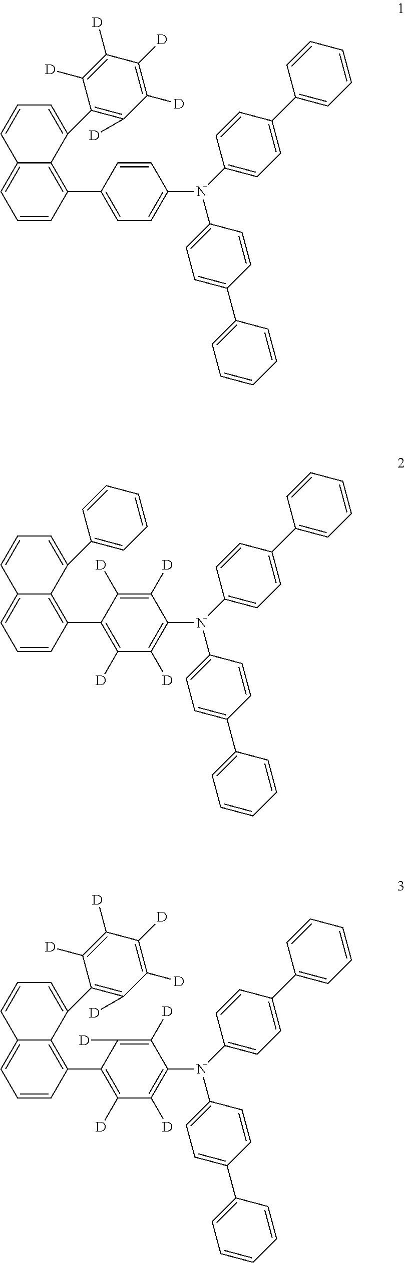 [SMILES: [2H]c1c([2H])c([2H])c(-c2cccc3cccc(-c4ccc(N(c5ccc(-c6ccccc6)cc5)c5ccc(-c6ccccc6)cc5)cc4)c23)c([2H])c1[2H] ; [2H]c1c([2H])c(N(c2ccc(-c3ccccc3)cc2)c2ccc(-c3ccccc3)cc2)c([2H])c([2H])c1-c1cccc2cccc(-c3ccccc3)c12 ; [2H]c1c([2H])c([2H])c(-c2cccc3cccc(-c4c([2H])c([2H])c(N(c5ccc(-c6ccccc6)cc5)c5ccc(-c6ccccc6)cc5)c([2H])c4[2H])c23)c([2H])c1[2H]]

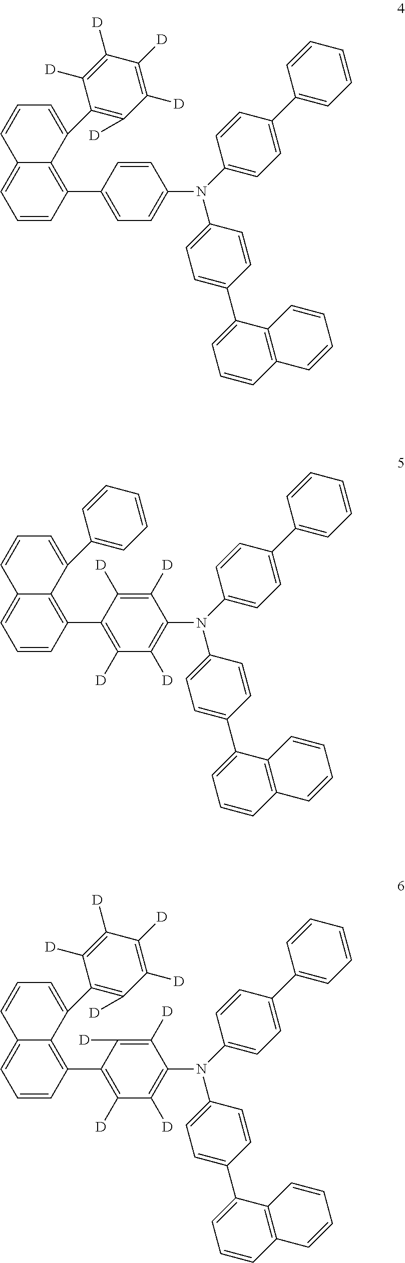 [SMILES: [2H]c1c([2H])c([2H])c(-c2cccc3cccc(-c4ccc(N(c5ccc(-c6ccccc6)cc5)c5ccc(-c6cccc7ccccc67)cc5)cc4)c23)c([2H])c1[2H] ; [2H]c1c([2H])c(N(c2ccc(-c3ccccc3)cc2)c2ccc(-c3cccc4ccccc34)cc2)c([2H])c([2H])c1-c1cccc2cccc(-c3ccccc3)c12 ; [2H]c1c([2H])c([2H])c(-c2cccc3cccc(-c4c([2H])c([2H])c(N(c5ccc(-c6ccccc6)cc5)c5ccc(-c6cccc7ccccc67)cc5)c([2H])c4[2H])c23)c([2H])c1[2H]]

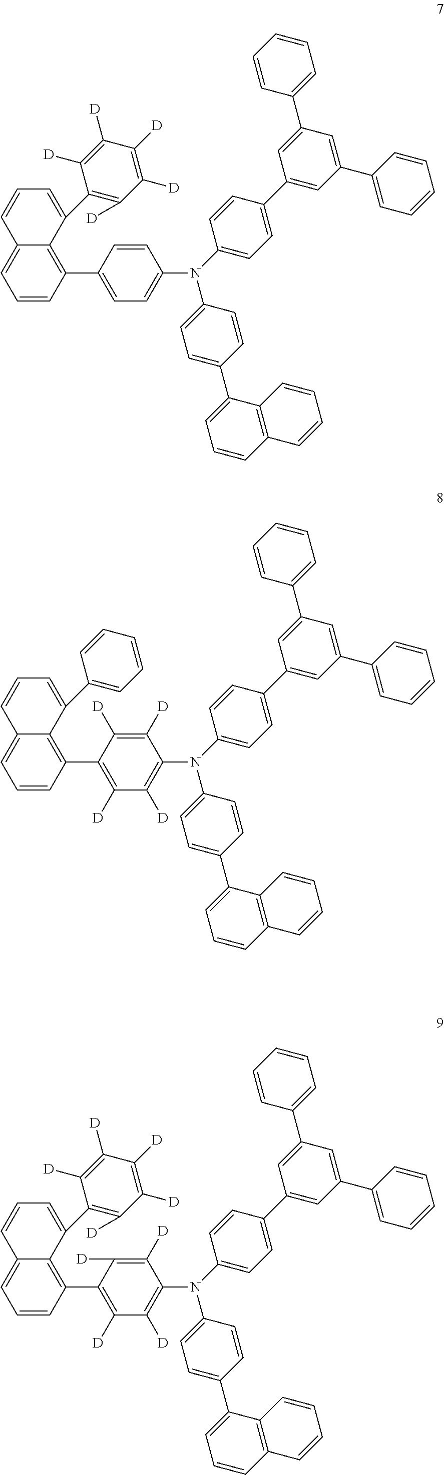 [SMILES: [2H]c1c([2H])c([2H])c(-c2cccc3cccc(-c4ccc(N(c5ccc(-c6cc(-c7ccccc7)cc(-c7ccccc7)c6)cc5)c5ccc(-c6cccc7ccccc67)cc5)cc4)c23)c([2H])c1[2H] ; [2H]c1c([2H])c(N(c2ccc(-c3cc(-c4ccccc4)cc(-c4ccccc4)c3)cc2)c2ccc(-c3cccc4ccccc34)cc2)c([2H])c([2H])c1-c1cccc2cccc(-c3ccccc3)c12 ; [2H]c1c([2H])c([2H])c(-c2cccc3cccc(-c4c([2H])c([2H])c(N(c5ccc(-c6cc(-c7ccccc7)cc(-c7ccccc7)c6)cc5)c5ccc(-c6cccc7ccccc67)cc5)c([2H])c4[2H])c23)c([2H])c1[2H]]

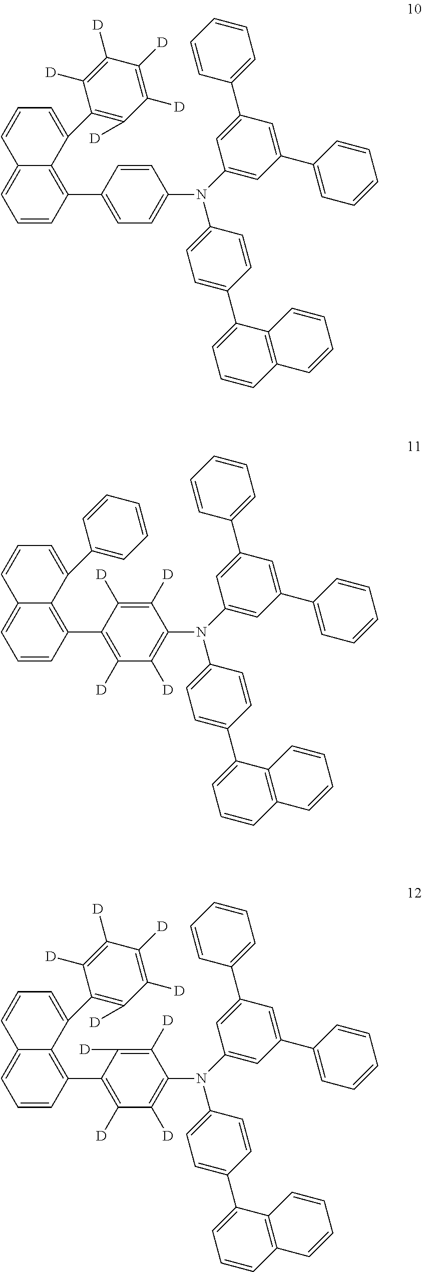 [SMILES: [2H]c1c([2H])c([2H])c(-c2cccc3cccc(-c4ccc(N(c5ccc(-c6cccc7ccccc67)cc5)c5cc(-c6ccccc6)cc(-c6ccccc6)c5)cc4)c23)c([2H])c1[2H] ; [2H]c1c([2H])c(N(c2ccc(-c3cccc4ccccc34)cc2)c2cc(-c3ccccc3)cc(-c3ccccc3)c2)c([2H])c([2H])c1-c1cccc2cccc(-c3ccccc3)c12 ; [2H]c1c([2H])c([2H])c(-c2cccc3cccc(-c4c([2H])c([2H])c(N(c5ccc(-c6cccc7ccccc67)cc5)c5cc(-c6ccccc6)cc(-c6ccccc6)c5)c([2H])c4[2H])c23)c([2H])c1[2H]]

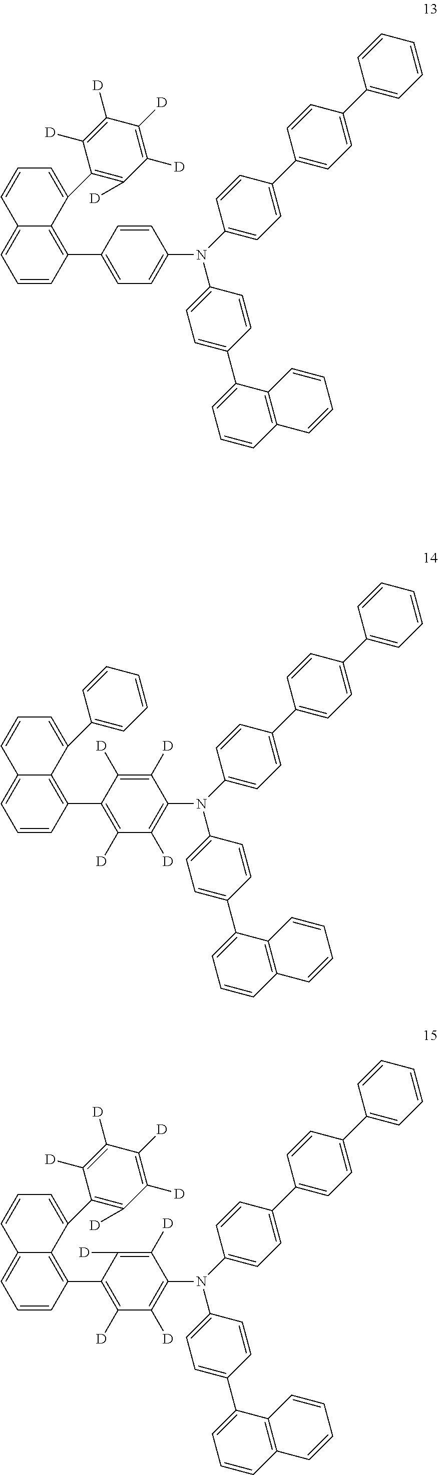 [SMILES: [2H]c1c([2H])c([2H])c(-c2cccc3cccc(-c4ccc(N(c5ccc(-c6ccc(-c7ccccc7)cc6)cc5)c5ccc(-c6cccc7ccccc67)cc5)cc4)c23)c([2H])c1[2H] ; [2H]c1c([2H])c(N(c2ccc(-c3ccc(-c4ccccc4)cc3)cc2)c2ccc(-c3cccc4ccccc34)cc2)c([2H])c([2H])c1-c1cccc2cccc(-c3ccccc3)c12 ; [2H]c1c([2H])c([2H])c(-c2cccc3cccc(-c4c([2H])c([2H])c(N(c5ccc(-c6ccc(-c7ccccc7)cc6)cc5)c5ccc(-c6cccc7ccccc67)cc5)c([2H])c4[2H])c23)c([2H])c1[2H]]

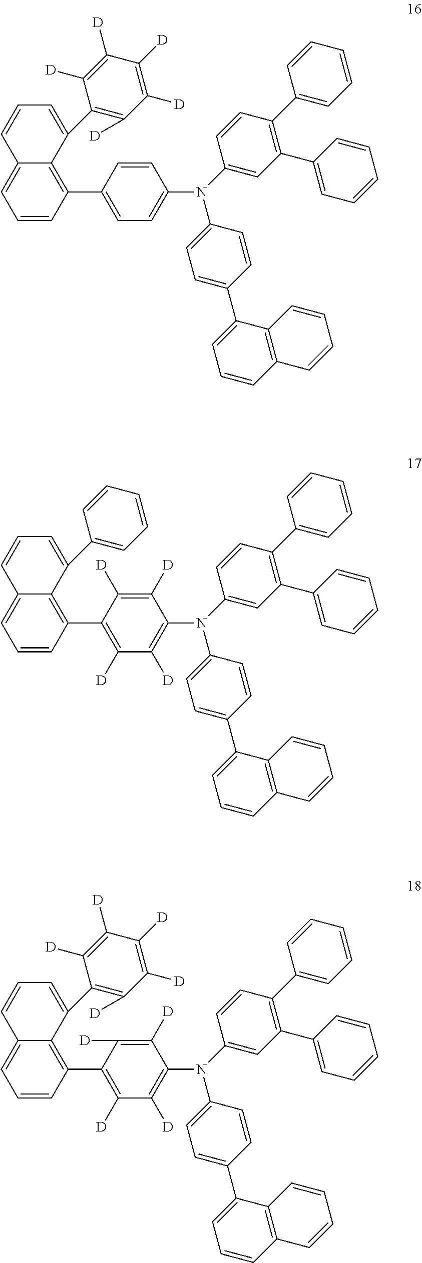 [SMILES: [2H]c1c([2H])c([2H])c(-c2cccc3cccc(-c4ccc(N(c5ccc(-c6cccc7ccccc67)cc5)c5ccc(-c6ccccc6)c(-c6ccccc6)c5)cc4)c23)c([2H])c1[2H] ; [2H]c1c([2H])c(N(c2ccc(-c3cccc4ccccc34)cc2)c2ccc(-c3ccccc3)c(-c3ccccc3)c2)c([2H])c([2H])c1-c1cccc2cccc(-c3ccccc3)c12 ; [2H]c1c([2H])c([2H])c(-c2cccc3cccc(-c4c([2H])c([2H])c(N(c5ccc(-c6cccc7ccccc67)cc5)c5ccc(-c6ccccc6)c(-c6ccccc6)c5)c([2H])c4[2H])c23)c([2H])c1[2H]]

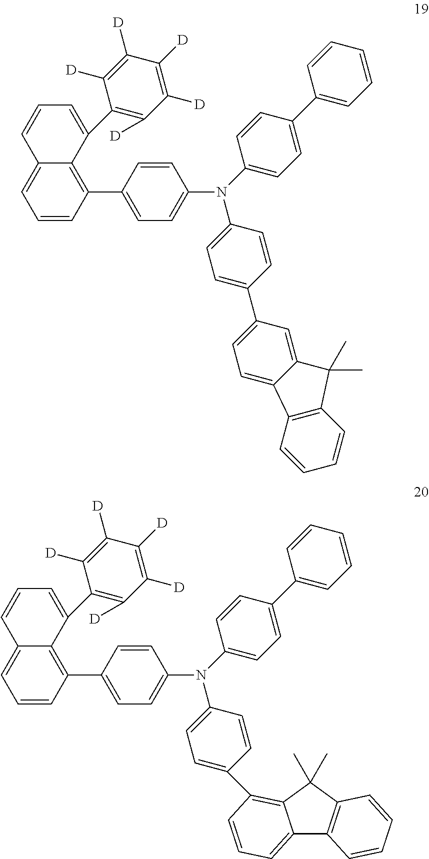 [SMILES: [2H]c1c([2H])c([2H])c(-c2cccc3cccc(-c4ccc(N(c5ccc(-c6ccccc6)cc5)c5ccc(-c6ccc7c(c6)C(C)(C)c6ccccc6-7)cc5)cc4)c23)c([2H])c1[2H] ; [2H]c1c([2H])c([2H])c(-c2cccc3cccc(-c4ccc(N(c5ccc(-c6ccccc6)cc5)c5ccc(-c6cccc7c6C(C)(C)c6ccccc6-7)cc5)cc4)c23)c([2H])c1[2H]]

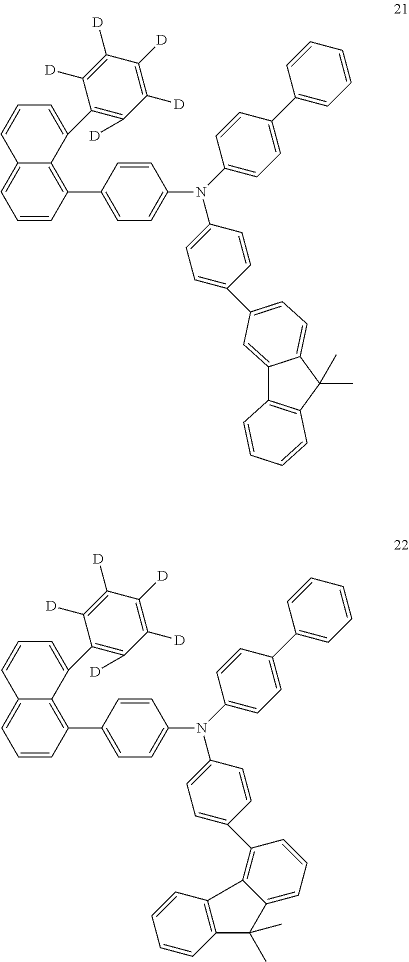 [SMILES: [2H]c1c([2H])c([2H])c(-c2cccc3cccc(-c4ccc(N(c5ccc(-c6ccccc6)cc5)c5ccc(-c6ccc7c(c6)-c6ccccc6C7(C)C)cc5)cc4)c23)c([2H])c1[2H] ; [2H]c1c([2H])c([2H])c(-c2cccc3cccc(-c4ccc(N(c5ccc(-c6ccccc6)cc5)c5ccc(-c6cccc7c6-c6ccccc6C7(C)C)cc5)cc4)c23)c([2H])c1[2H]]

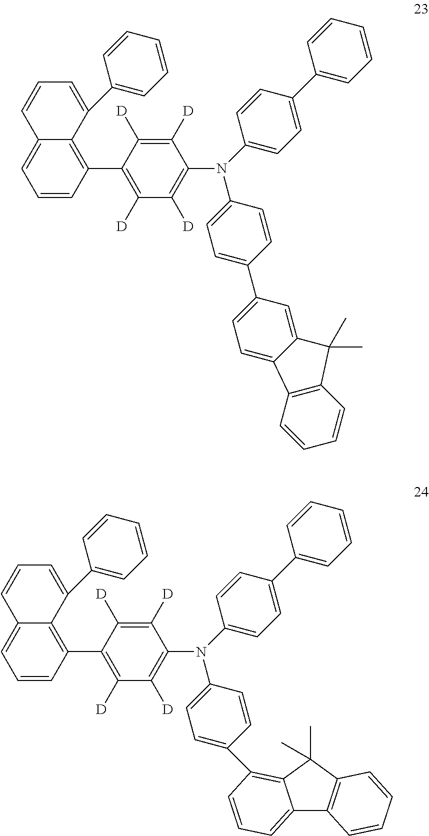 [SMILES: [2H]c1c([2H])c(N(c2ccc(-c3ccccc3)cc2)c2ccc(-c3ccc4c(c3)C(C)(C)c3ccccc3-4)cc2)c([2H])c([2H])c1-c1cccc2cccc(-c3ccccc3)c12 ; [2H]c1c([2H])c(N(c2ccc(-c3ccccc3)cc2)c2ccc(-c3cccc4c3C(C)(C)c3ccccc3-4)cc2)c([2H])c([2H])c1-c1cccc2cccc(-c3ccccc3)c12]

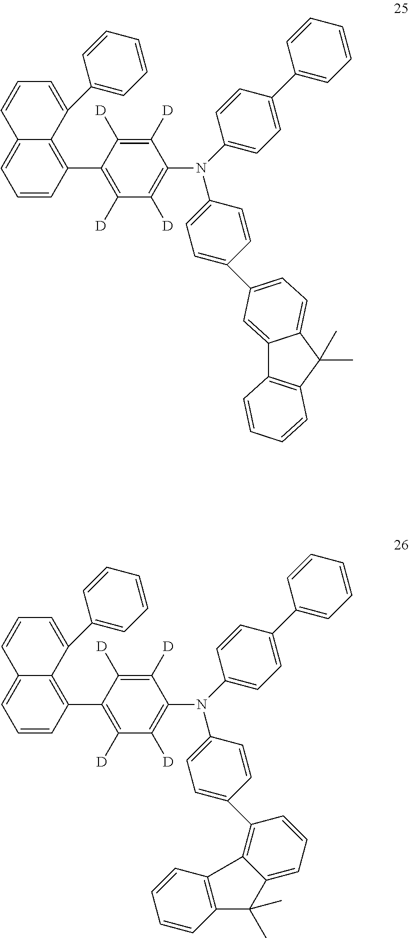 [SMILES: [2H]c1c([2H])c(N(c2ccc(-c3ccccc3)cc2)c2ccc(-c3ccc4c(c3)-c3ccccc3C4(C)C)cc2)c([2H])c([2H])c1-c1cccc2cccc(-c3ccccc3)c12 ; [2H]c1c([2H])c(N(c2ccc(-c3ccccc3)cc2)c2ccc(-c3cccc4c3-c3ccccc3C4(C)C)cc2)c([2H])c([2H])c1-c1cccc2cccc(-c3ccccc3)c12]

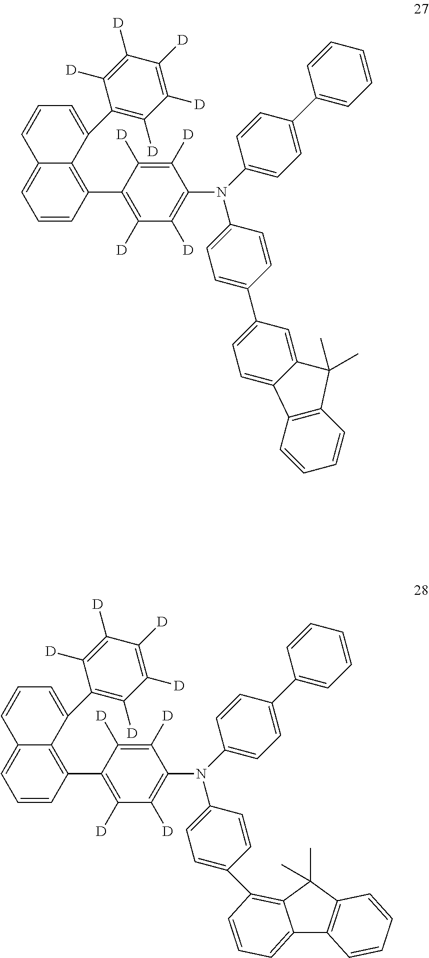 [SMILES: [2H]c1c([2H])c([2H])c(-c2cccc3cccc(-c4c([2H])c([2H])c(N(c5ccc(-c6ccccc6)cc5)c5ccc(-c6ccc7c(c6)C(C)(C)c6ccccc6-7)cc5)c([2H])c4[2H])c23)c([2H])c1[2H] ; [2H]c1c([2H])c([2H])c(-c2cccc3cccc(-c4c([2H])c([2H])c(N(c5ccc(-c6ccccc6)cc5)c5ccc(-c6cccc7c6C(C)(C)c6ccccc6-7)cc5)c([2H])c4[2H])c23)c([2H])c1[2H]]

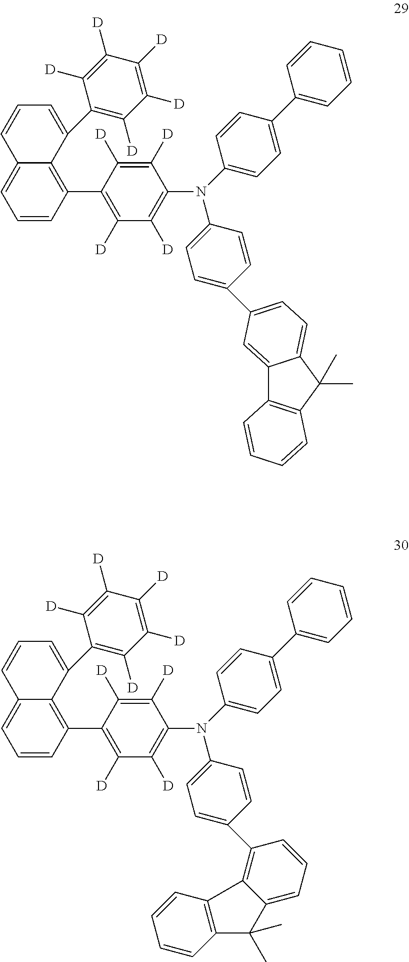 [SMILES: [2H]c1c([2H])c([2H])c(-c2cccc3cccc(-c4c([2H])c([2H])c(N(c5ccc(-c6ccccc6)cc5)c5ccc(-c6ccc7c(c6)-c6ccccc6C7(C)C)cc5)c([2H])c4[2H])c23)c([2H])c1[2H] ; [2H]c1c([2H])c([2H])c(-c2cccc3cccc(-c4c([2H])c([2H])c(N(c5ccc(-c6ccccc6)cc5)c5ccc(-c6cccc7c6-c6ccccc6C7(C)C)cc5)c([2H])c4[2H])c23)c([2H])c1[2H]]

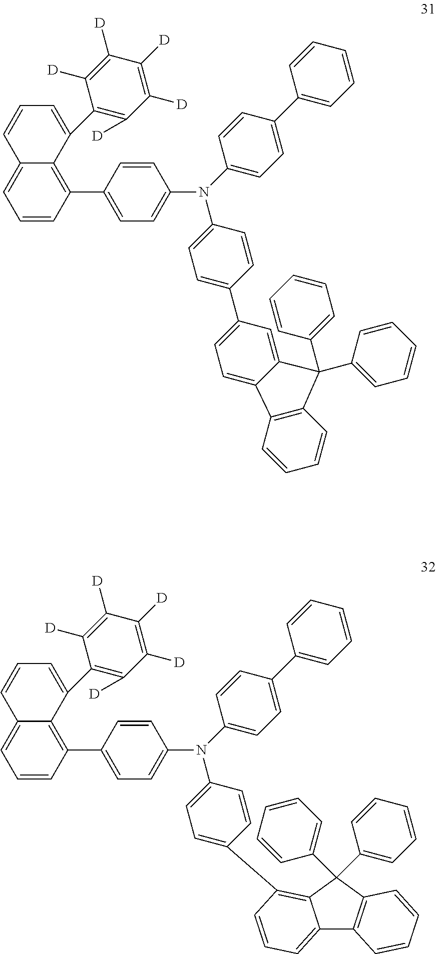 [SMILES: [2H]c1c([2H])c([2H])c(-c2cccc3cccc(-c4ccc(N(c5ccc(-c6ccccc6)cc5)c5ccc(-c6ccc7c(c6)C(c6ccccc6)(c6ccccc6)c6ccccc6-7)cc5)cc4)c23)c([2H])c1[2H] ; [2H]c1c([2H])c([2H])c(-c2cccc3cccc(-c4ccc(N(c5ccc(-c6ccccc6)cc5)c5ccc(-c6cccc7c6C(c6ccccc6)(c6ccccc6)c6ccccc6-7)cc5)cc4)c23)c([2H])c1[2H]]

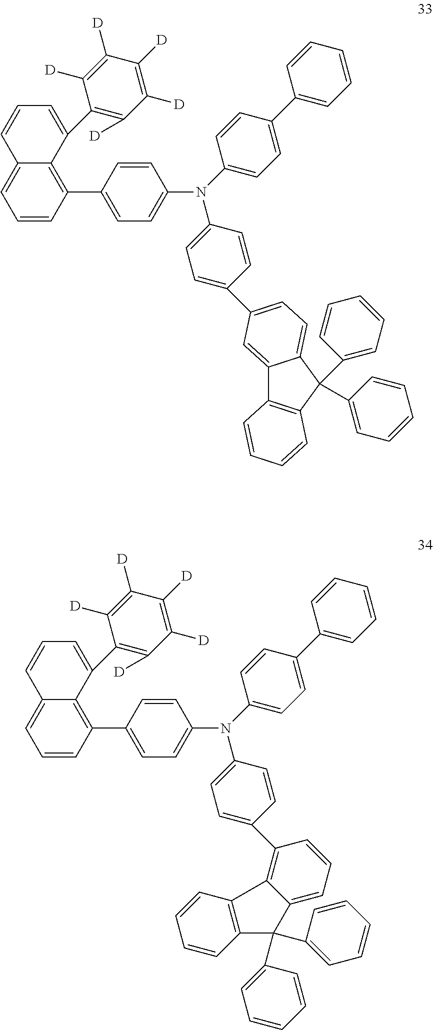 [SMILES: [2H]c1c([2H])c([2H])c(-c2cccc3cccc(-c4ccc(N(c5ccc(-c6ccccc6)cc5)c5ccc(-c6ccc7c(c6)-c6ccccc6C7(c6ccccc6)c6ccccc6)cc5)cc4)c23)c([2H])c1[2H] ; [2H]c1c([2H])c([2H])c(-c2cccc3cccc(-c4ccc(N(c5ccc(-c6ccccc6)cc5)c5ccc(-c6cccc7c6-c6ccccc6C7(c6ccccc6)c6ccccc6)cc5)cc4)c23)c([2H])c1[2H]]

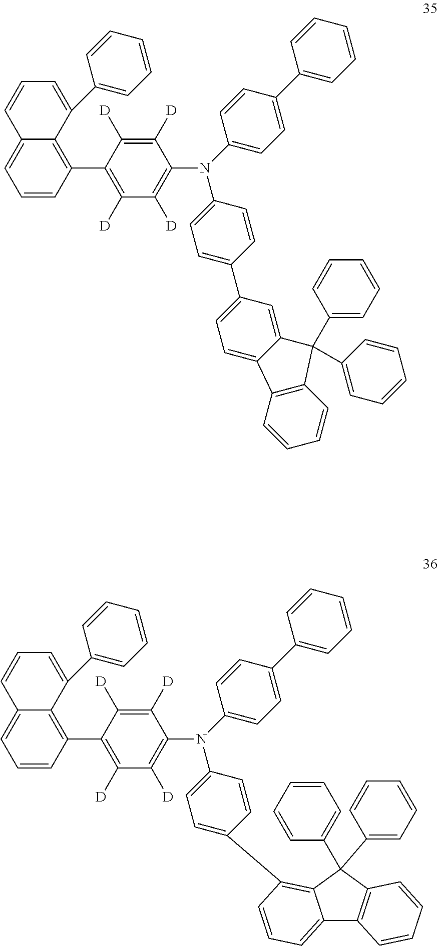 [SMILES: [2H]c1c([2H])c(N(c2ccc(-c3ccccc3)cc2)c2ccc(-c3ccc4c(c3)C(c3ccccc3)(c3ccccc3)c3ccccc3-4)cc2)c([2H])c([2H])c1-c1cccc2cccc(-c3ccccc3)c12 ; [2H]c1c([2H])c(N(c2ccc(-c3ccccc3)cc2)c2ccc(-c3cccc4c3C(c3ccccc3)(c3ccccc3)c3ccccc3-4)cc2)c([2H])c([2H])c1-c1cccc2cccc(-c3ccccc3)c12]

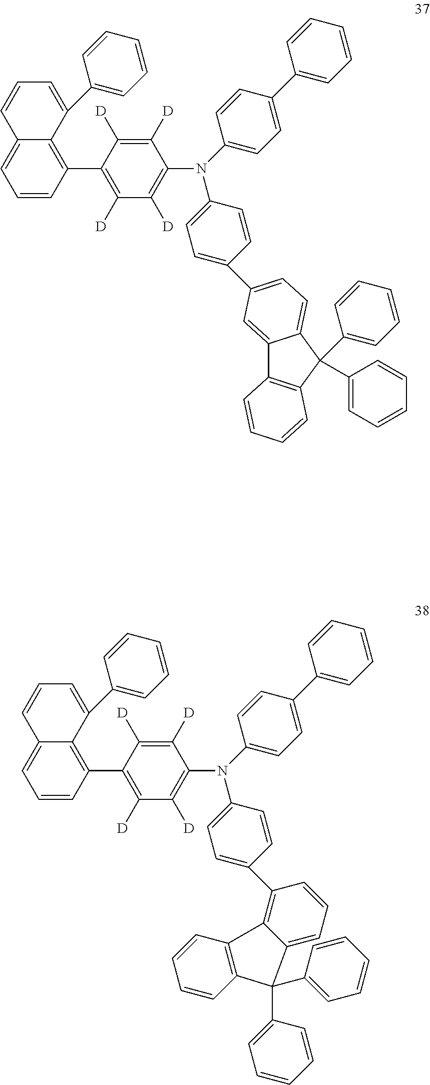 [SMILES: [2H]c1c([2H])c(N(c2ccc(-c3ccccc3)cc2)c2ccc(-c3ccc4c(c3)-c3ccccc3C4(c3ccccc3)c3ccccc3)cc2)c([2H])c([2H])c1-c1cccc2cccc(-c3ccccc3)c12 ; [2H]c1c([2H])c(N(c2ccc(-c3ccccc3)cc2)c2ccc(-c3cccc4c3-c3ccccc3C4(c3ccccc3)c3ccccc3)cc2)c([2H])c([2H])c1-c1cccc2cccc(-c3ccccc3)c12]

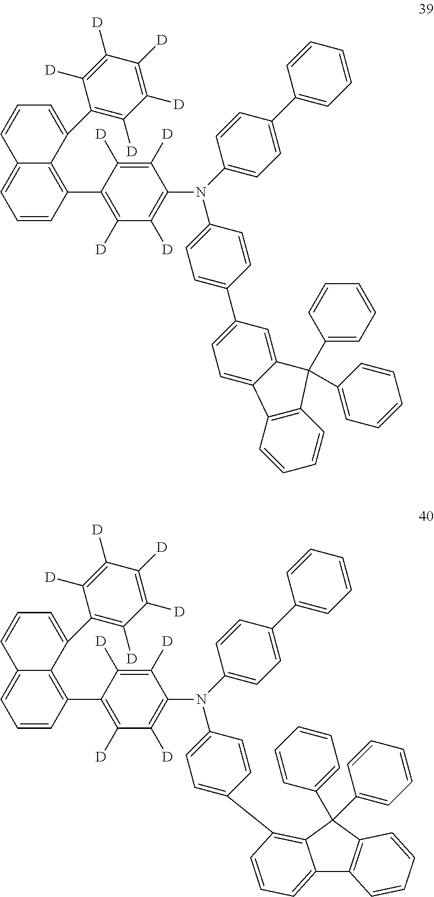 [SMILES: [2H]c1c([2H])c([2H])c(-c2cccc3cccc(-c4c([2H])c([2H])c(N(c5ccc(-c6ccccc6)cc5)c5ccc(-c6ccc7c(c6)C(c6ccccc6)(c6ccccc6)c6ccccc6-7)cc5)c([2H])c4[2H])c23)c([2H])c1[2H] ; [2H]c1c([2H])c([2H])c(-c2cccc3cccc(-c4c([2H])c([2H])c(N(c5ccc(-c6ccccc6)cc5)c5ccc(-c6cccc7c6C(c6ccccc6)(c6ccccc6)c6ccccc6-7)cc5)c([2H])c4[2H])c23)c([2H])c1[2H]]

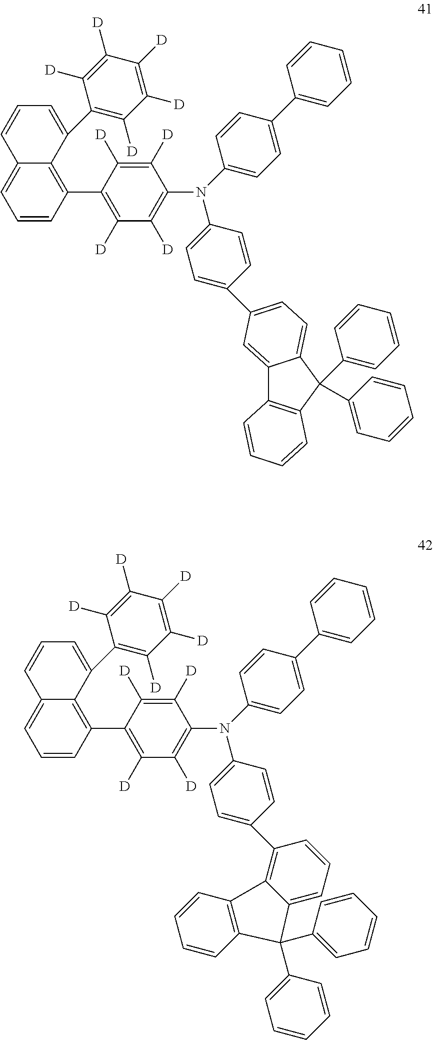 [SMILES: [2H]c1c([2H])c([2H])c(-c2cccc3cccc(-c4c([2H])c([2H])c(N(c5ccc(-c6ccccc6)cc5)c5ccc(-c6ccc7c(c6)-c6ccccc6C7(c6ccccc6)c6ccccc6)cc5)c([2H])c4[2H])c23)c([2H])c1[2H] ; [2H]c1c([2H])c([2H])c(-c2cccc3cccc(-c4c([2H])c([2H])c(N(c5ccc(-c6ccccc6)cc5)c5ccc(-c6cccc7c6-c6ccccc6C7(c6ccccc6)c6ccccc6)cc5)c([2H])c4[2H])c23)c([2H])c1[2H]]

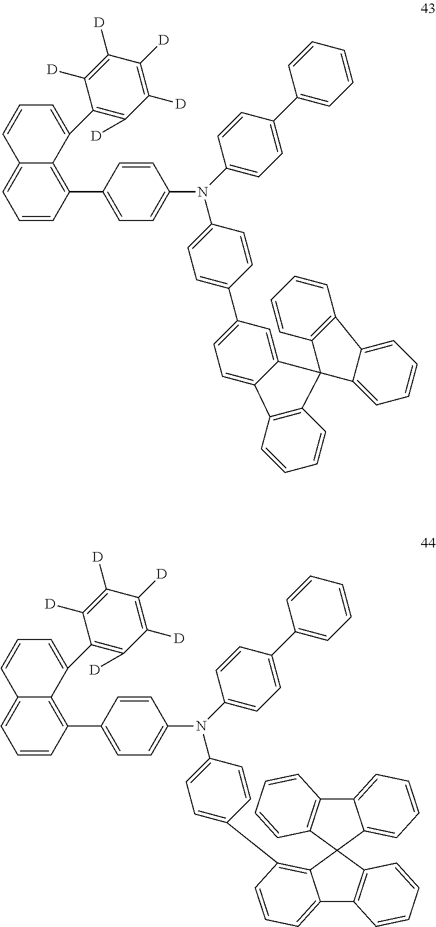 [SMILES: [2H]c1c([2H])c([2H])c(-c2cccc3cccc(-c4ccc(N(c5ccc(-c6ccccc6)cc5)c5ccc(-c6ccc7c(c6)C6(c8ccccc8-c8ccccc86)c6ccccc6-7)cc5)cc4)c23)c([2H])c1[2H] ; [2H]c1c([2H])c([2H])c(-c2cccc3cccc(-c4ccc(N(c5ccc(-c6ccccc6)cc5)c5ccc(-c6cccc7c6C6(c8ccccc8-c8ccccc86)c6ccccc6-7)cc5)cc4)c23)c([2H])c1[2H]]

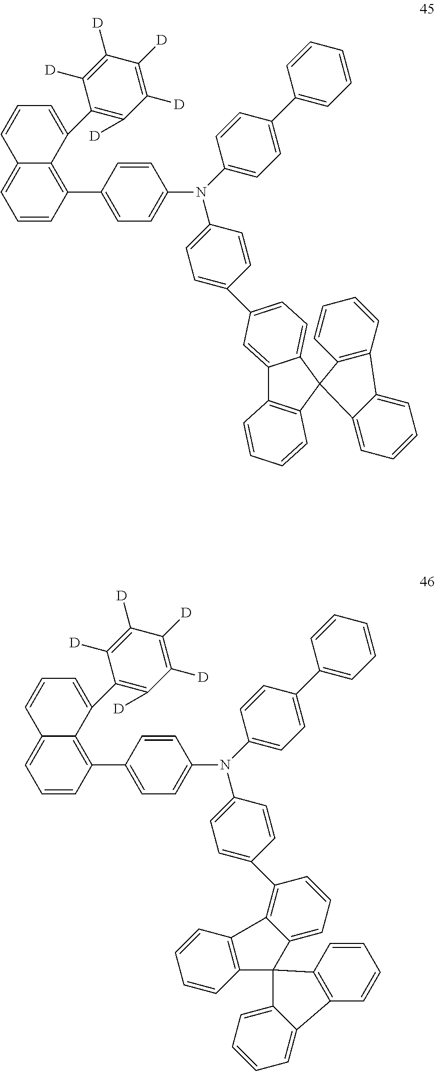 [SMILES: [2H]c1c([2H])c([2H])c(-c2cccc3cccc(-c4ccc(N(c5ccc(-c6ccccc6)cc5)c5ccc(-c6ccc7c(c6)-c6ccccc6C76c7ccccc7-c7ccccc76)cc5)cc4)c23)c([2H])c1[2H] ; [2H]c1c([2H])c([2H])c(-c2cccc3cccc(-c4ccc(N(c5ccc(-c6ccccc6)cc5)c5ccc(-c6cccc7c6-c6ccccc6C76c7ccccc7-c7ccccc76)cc5)cc4)c23)c([2H])c1[2H]]

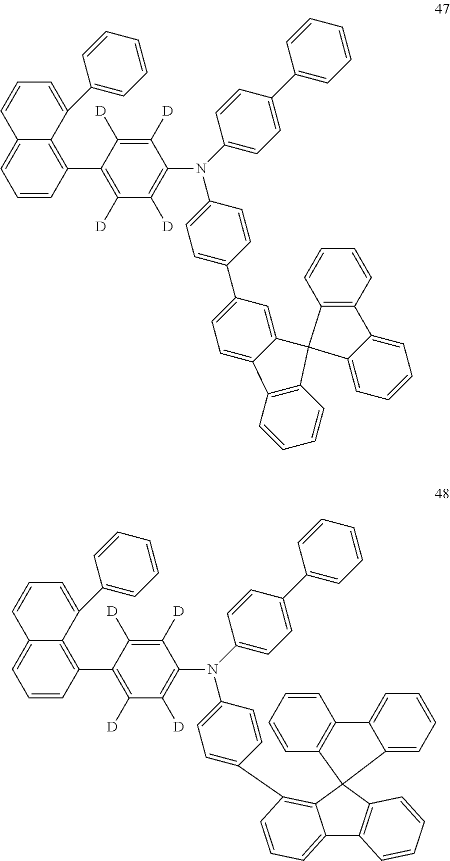 [SMILES: [2H]c1c([2H])c(N(c2ccc(-c3ccccc3)cc2)c2ccc(-c3ccc4c(c3)C3(c5ccccc5-c5ccccc53)c3ccccc3-4)cc2)c([2H])c([2H])c1-c1cccc2cccc(-c3ccccc3)c12 ; [2H]c1c([2H])c(N(c2ccc(-c3ccccc3)cc2)c2ccc(-c3cccc4c3C3(c5ccccc5-c5ccccc53)c3ccccc3-4)cc2)c([2H])c([2H])c1-c1cccc2cccc(-c3ccccc3)c12]

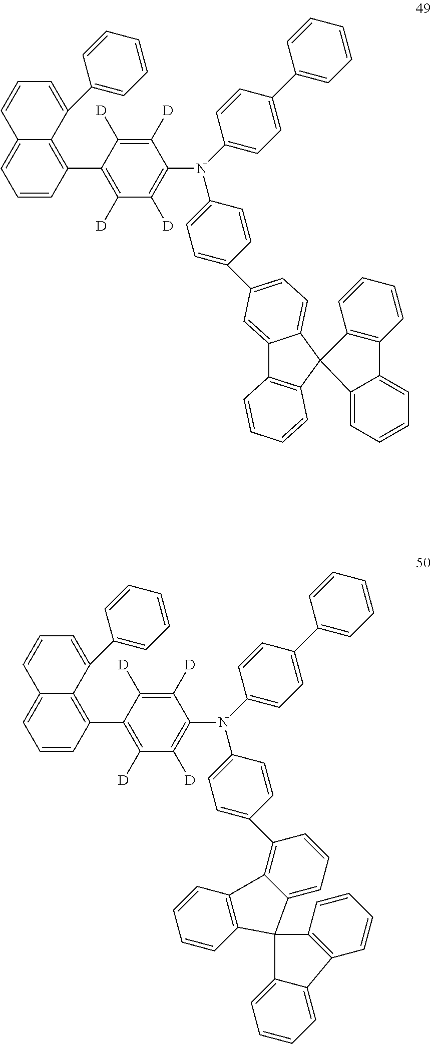 [SMILES: [2H]c1c([2H])c(N(c2ccc(-c3ccccc3)cc2)c2ccc(-c3ccc4c(c3)-c3ccccc3C43c4ccccc4-c4ccccc43)cc2)c([2H])c([2H])c1-c1cccc2cccc(-c3ccccc3)c12 ; [2H]c1c([2H])c(N(c2ccc(-c3ccccc3)cc2)c2ccc(-c3cccc4c3-c3ccccc3C43c4ccccc4-c4ccccc43)cc2)c([2H])c([2H])c1-c1cccc2cccc(-c3ccccc3)c12]

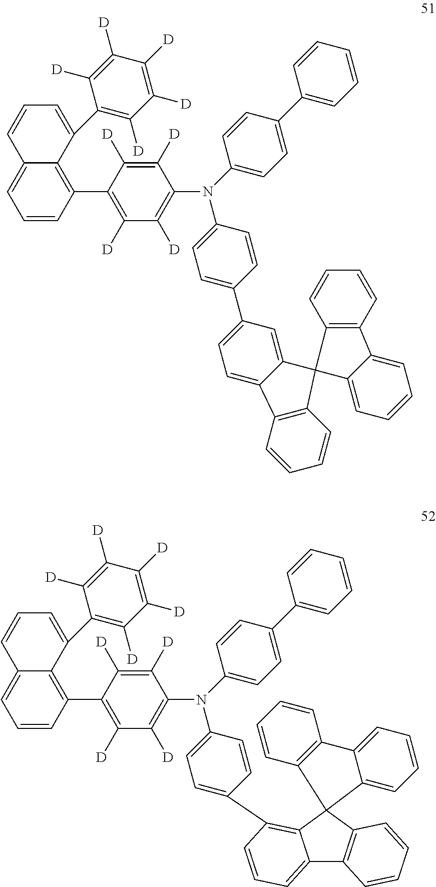 [SMILES: [2H]c1c([2H])c([2H])c(-c2cccc3cccc(-c4c([2H])c([2H])c(N(c5ccc(-c6ccccc6)cc5)c5ccc(-c6ccc7c(c6)C6(c8ccccc8-c8ccccc86)c6ccccc6-7)cc5)c([2H])c4[2H])c23)c([2H])c1[2H] ; [2H]c1c([2H])c([2H])c(-c2cccc3cccc(-c4c([2H])c([2H])c(N(c5ccc(-c6ccccc6)cc5)c5ccc(-c6cccc7c6C6(c8ccccc8-c8ccccc86)c6ccccc6-7)cc5)c([2H])c4[2H])c23)c([2H])c1[2H]]

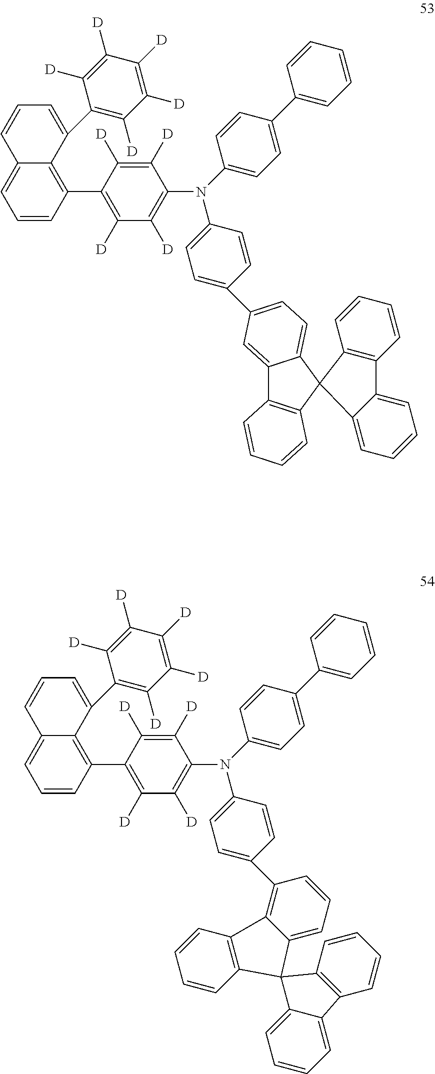 [SMILES: [2H]c1c([2H])c([2H])c(-c2cccc3cccc(-c4c([2H])c([2H])c(N(c5ccc(-c6ccccc6)cc5)c5ccc(-c6ccc7c(c6)-c6ccccc6C76c7ccccc7-c7ccccc76)cc5)c([2H])c4[2H])c23)c([2H])c1[2H] ; [2H]c1c([2H])c([2H])c(-c2cccc3cccc(-c4c([2H])c([2H])c(N(c5ccc(-c6ccccc6)cc5)c5ccc(-c6cccc7c6-c6ccccc6C76c7ccccc7-c7ccccc76)cc5)c([2H])c4[2H])c23)c([2H])c1[2H]]

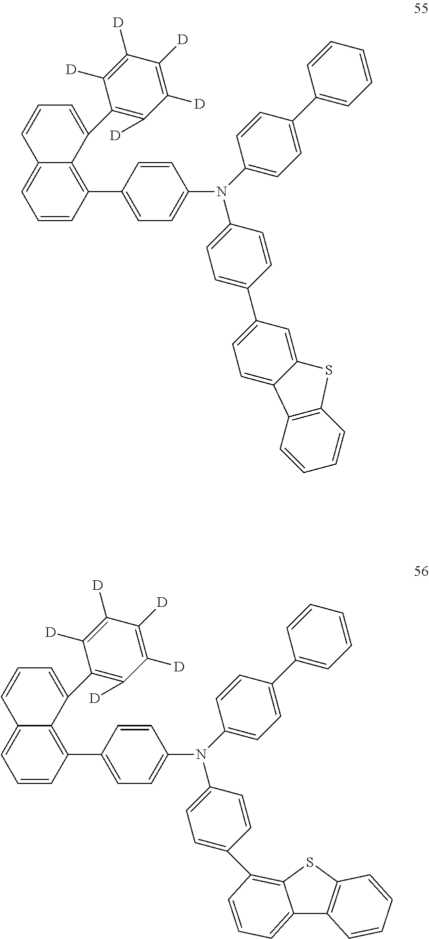 [SMILES: [2H]c1c([2H])c([2H])c(-c2cccc3cccc(-c4ccc(N(c5ccc(-c6ccccc6)cc5)c5ccc(-c6ccc7c(c6)sc6ccccc67)cc5)cc4)c23)c([2H])c1[2H] ; [2H]c1c([2H])c([2H])c(-c2cccc3cccc(-c4ccc(N(c5ccc(-c6ccccc6)cc5)c5ccc(-c6cccc7c6sc6ccccc67)cc5)cc4)c23)c([2H])c1[2H]]

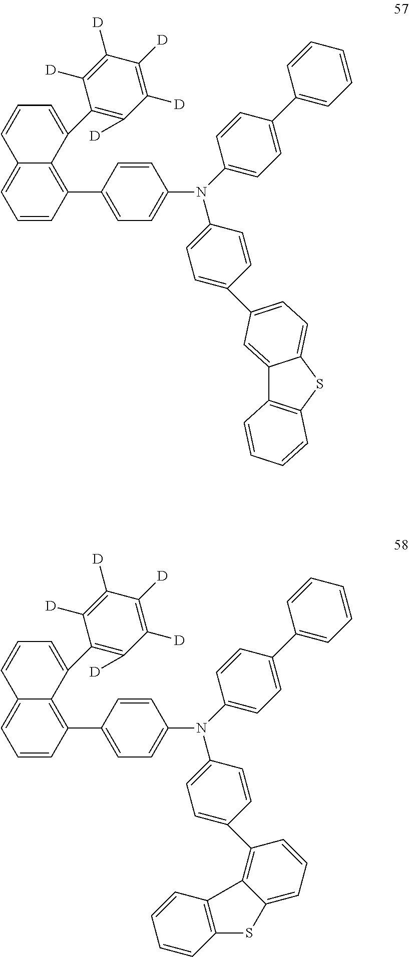 [SMILES: [2H]c1c([2H])c([2H])c(-c2cccc3cccc(-c4ccc(N(c5ccc(-c6ccccc6)cc5)c5ccc(-c6ccc7sc8ccccc8c7c6)cc5)cc4)c23)c([2H])c1[2H] ; [2H]c1c([2H])c([2H])c(-c2cccc3cccc(-c4ccc(N(c5ccc(-c6ccccc6)cc5)c5ccc(-c6cccc7sc8ccccc8c67)cc5)cc4)c23)c([2H])c1[2H]]

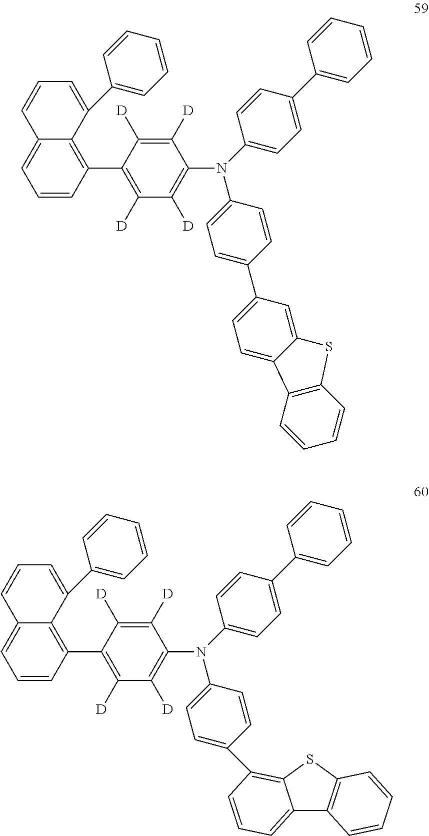 [SMILES: [2H]c1c([2H])c(N(c2ccc(-c3ccccc3)cc2)c2ccc(-c3ccc4c(c3)sc3ccccc34)cc2)c([2H])c([2H])c1-c1cccc2cccc(-c3ccccc3)c12 ; [2H]c1c([2H])c(N(c2ccc(-c3ccccc3)cc2)c2ccc(-c3cccc4c3sc3ccccc34)cc2)c([2H])c([2H])c1-c1cccc2cccc(-c3ccccc3)c12]

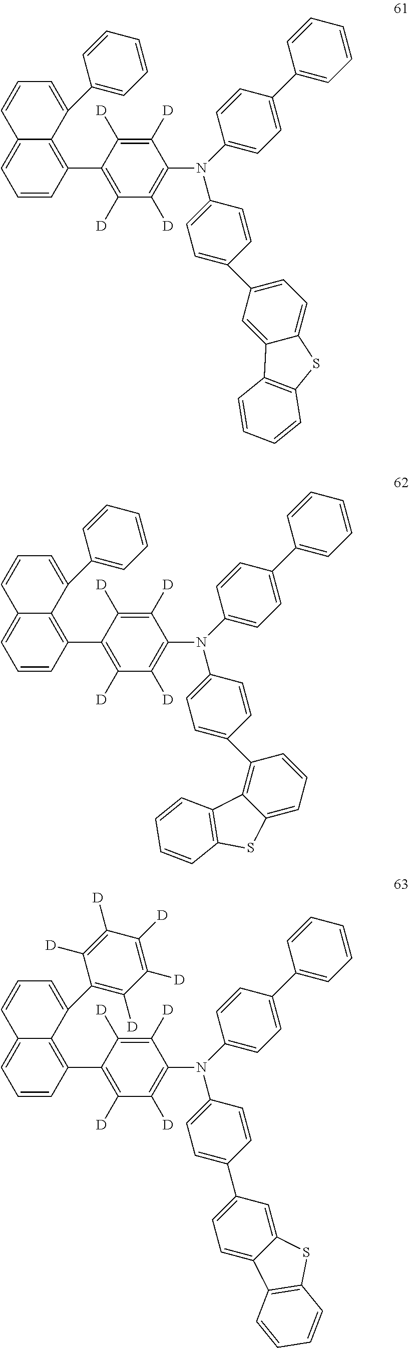 [SMILES: [2H]c1c([2H])c(N(c2ccc(-c3ccccc3)cc2)c2ccc(-c3ccc4sc5ccccc5c4c3)cc2)c([2H])c([2H])c1-c1cccc2cccc(-c3ccccc3)c12 ; [2H]c1c([2H])c(N(c2ccc(-c3ccccc3)cc2)c2ccc(-c3cccc4sc5ccccc5c34)cc2)c([2H])c([2H])c1-c1cccc2cccc(-c3ccccc3)c12 ; [2H]c1c([2H])c([2H])c(-c2cccc3cccc(-c4c([2H])c([2H])c(N(c5ccc(-c6ccccc6)cc5)c5ccc(-c6ccc7c(c6)sc6ccccc67)cc5)c([2H])c4[2H])c23)c([2H])c1[2H]]

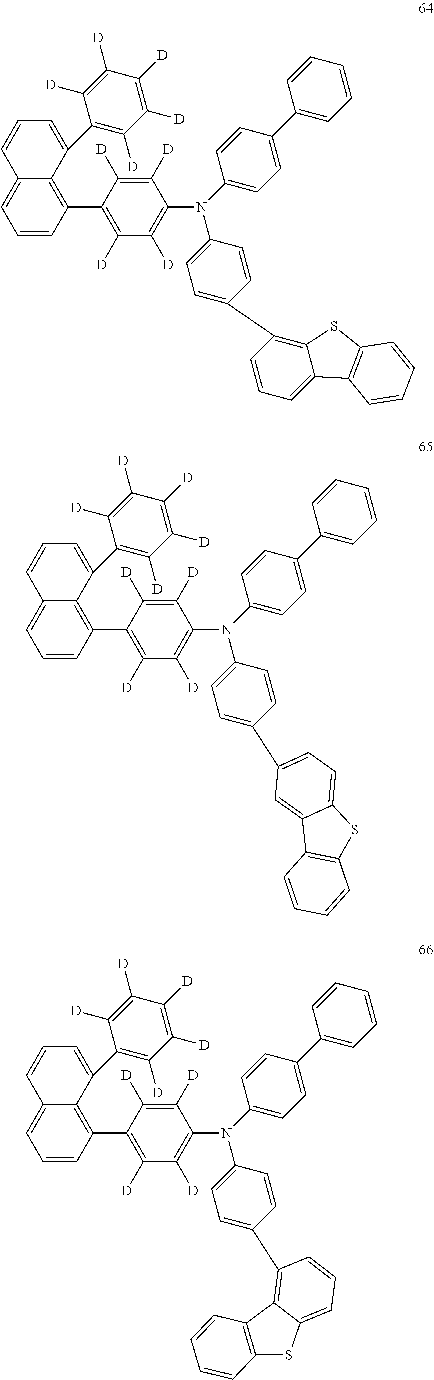 [SMILES: [2H]c1c([2H])c([2H])c(-c2cccc3cccc(-c4c([2H])c([2H])c(N(c5ccc(-c6ccccc6)cc5)c5ccc(-c6cccc7c6sc6ccccc67)cc5)c([2H])c4[2H])c23)c([2H])c1[2H] ; [2H]c1c([2H])c([2H])c(-c2cccc3cccc(-c4c([2H])c([2H])c(N(c5ccc(-c6ccccc6)cc5)c5ccc(-c6ccc7sc8ccccc8c7c6)cc5)c([2H])c4[2H])c23)c([2H])c1[2H] ; [2H]c1c([2H])c([2H])c(-c2cccc3cccc(-c4c([2H])c([2H])c(N(c5ccc(-c6ccccc6)cc5)c5ccc(-c6cccc7sc8ccccc8c67)cc5)c([2H])c4[2H])c23)c([2H])c1[2H]]

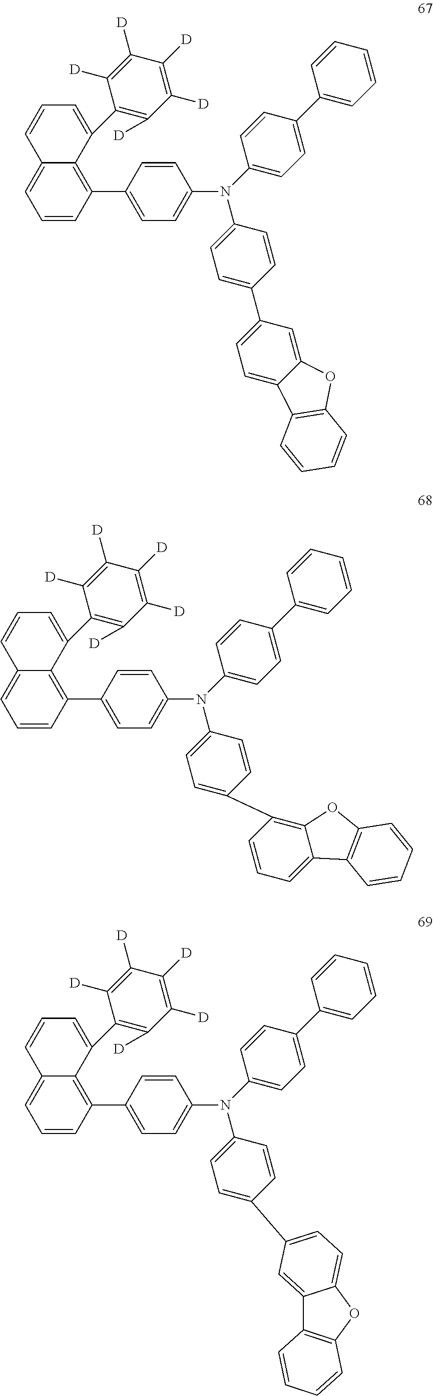 [SMILES: [2H]c1c([2H])c([2H])c(-c2cccc3cccc(-c4ccc(N(c5ccc(-c6ccccc6)cc5)c5ccc(-c6ccc7c(c6)oc6ccccc67)cc5)cc4)c23)c([2H])c1[2H] ; [2H]c1c([2H])c([2H])c(-c2cccc3cccc(-c4ccc(N(c5ccc(-c6ccccc6)cc5)c5ccc(-c6cccc7c6oc6ccccc67)cc5)cc4)c23)c([2H])c1[2H] ; [2H]c1c([2H])c([2H])c(-c2cccc3cccc(-c4ccc(N(c5ccc(-c6ccccc6)cc5)c5ccc(-c6ccc7oc8ccccc8c7c6)cc5)cc4)c23)c([2H])c1[2H]]

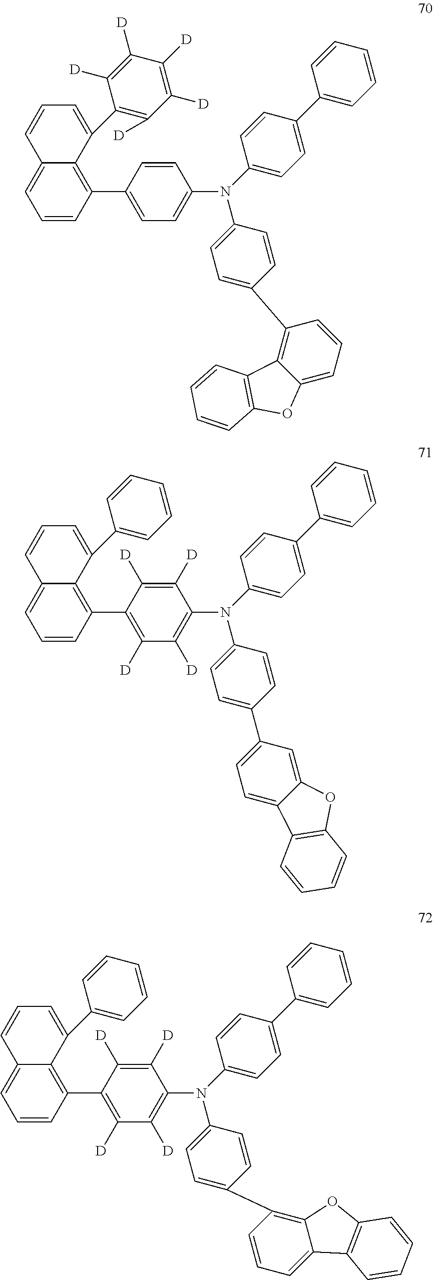 [SMILES: [2H]c1c([2H])c([2H])c(-c2cccc3cccc(-c4ccc(N(c5ccc(-c6ccccc6)cc5)c5ccc(-c6cccc7oc8ccccc8c67)cc5)cc4)c23)c([2H])c1[2H] ; [2H]c1c([2H])c(N(c2ccc(-c3ccccc3)cc2)c2ccc(-c3ccc4c(c3)oc3ccccc34)cc2)c([2H])c([2H])c1-c1cccc2cccc(-c3ccccc3)c12 ; [2H]c1c([2H])c(N(c2ccc(-c3ccccc3)cc2)c2ccc(-c3cccc4c3oc3ccccc34)cc2)c([2H])c([2H])c1-c1cccc2cccc(-c3ccccc3)c12]

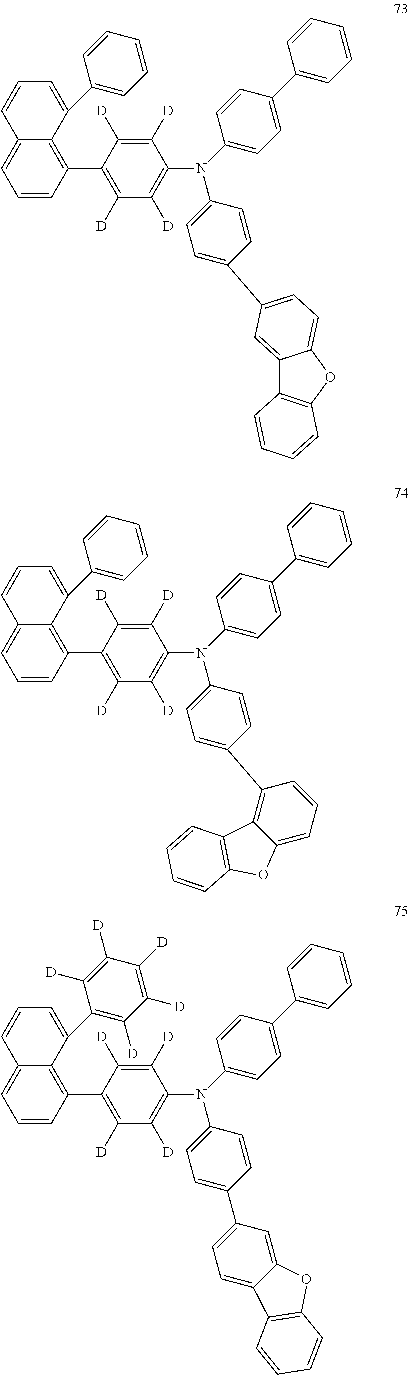 [SMILES: [2H]c1c([2H])c(N(c2ccc(-c3ccccc3)cc2)c2ccc(-c3ccc4oc5ccccc5c4c3)cc2)c([2H])c([2H])c1-c1cccc2cccc(-c3ccccc3)c12 ; [2H]c1c([2H])c(N(c2ccc(-c3ccccc3)cc2)c2ccc(-c3cccc4oc5ccccc5c34)cc2)c([2H])c([2H])c1-c1cccc2cccc(-c3ccccc3)c12 ; [2H]c1c([2H])c([2H])c(-c2cccc3cccc(-c4c([2H])c([2H])c(N(c5ccc(-c6ccccc6)cc5)c5ccc(-c6ccc7c(c6)oc6ccccc67)cc5)c([2H])c4[2H])c23)c([2H])c1[2H]]

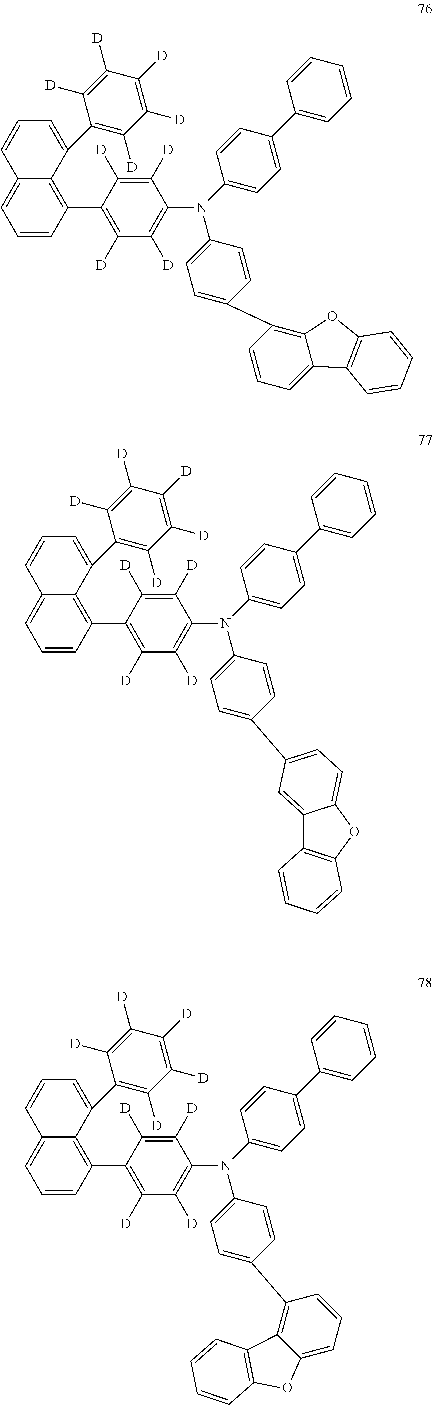 [SMILES: [2H]c1c([2H])c([2H])c(-c2cccc3cccc(-c4c([2H])c([2H])c(N(c5ccc(-c6ccccc6)cc5)c5ccc(-c6cccc7c6oc6ccccc67)cc5)c([2H])c4[2H])c23)c([2H])c1[2H] ; [2H]c1c([2H])c([2H])c(-c2cccc3cccc(-c4c([2H])c([2H])c(N(c5ccc(-c6ccccc6)cc5)c5ccc(-c6ccc7oc8ccccc8c7c6)cc5)c([2H])c4[2H])c23)c([2H])c1[2H] ; [2H]c1c([2H])c([2H])c(-c2cccc3cccc(-c4c([2H])c([2H])c(N(c5ccc(-c6ccccc6)cc5)c5ccc(-c6cccc7oc8ccccc8c67)cc5)c([2H])c4[2H])c23)c([2H])c1[2H]]

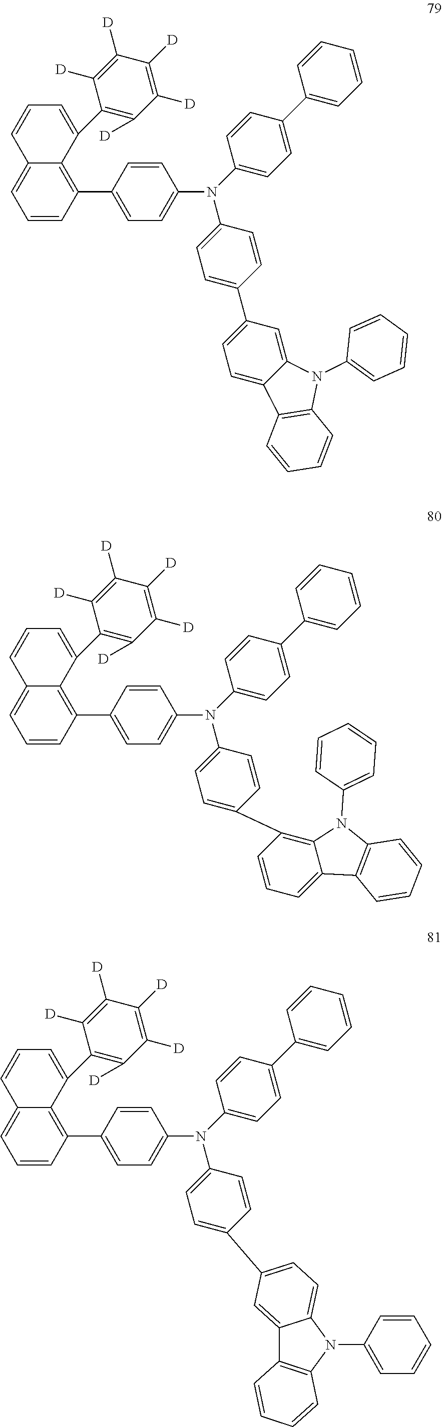 [SMILES: [2H]c1c([2H])c([2H])c(-c2cccc3cccc(-c4ccc(N(c5ccc(-c6ccccc6)cc5)c5ccc(-c6ccc7c8ccccc8n(-c8ccccc8)c7c6)cc5)cc4)c23)c([2H])c1[2H] ; [2H]c1c([2H])c([2H])c(-c2cccc3cccc(-c4ccc(N(c5ccc(-c6ccccc6)cc5)c5ccc(-c6cccc7c8ccccc8n(-c8ccccc8)c67)cc5)cc4)c23)c([2H])c1[2H] ; [2H]c1c([2H])c([2H])c(-c2cccc3cccc(-c4ccc(N(c5ccc(-c6ccccc6)cc5)c5ccc(-c6ccc7c(c6)c6ccccc6n7-c6ccccc6)cc5)cc4)c23)c([2H])c1[2H]]

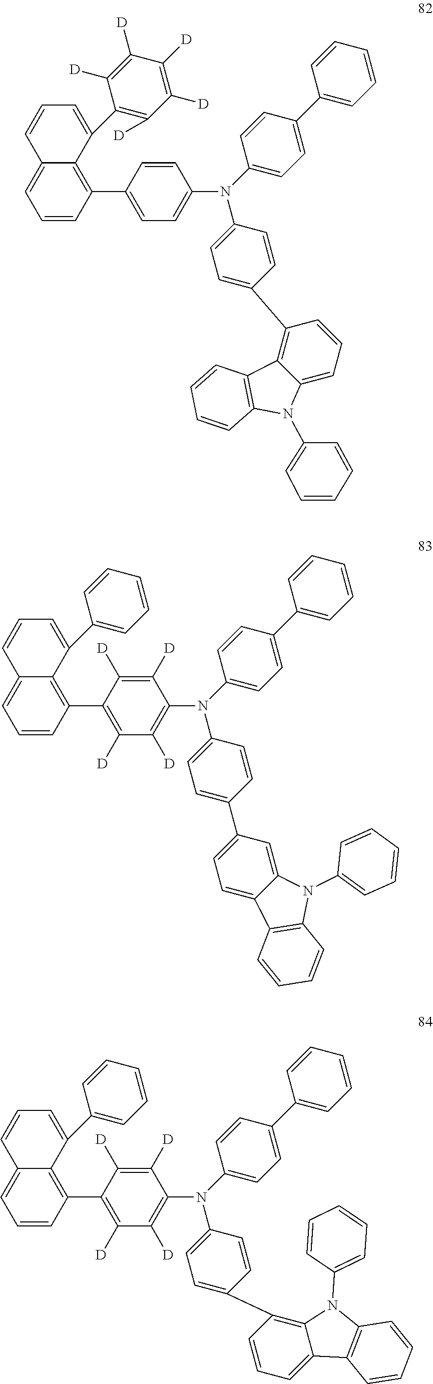 [SMILES: [2H]c1c([2H])c([2H])c(-c2cccc3cccc(-c4ccc(N(c5ccc(-c6ccccc6)cc5)c5ccc(-c6cccc7c6c6ccccc6n7-c6ccccc6)cc5)cc4)c23)c([2H])c1[2H] ; [2H]c1c([2H])c(N(c2ccc(-c3ccccc3)cc2)c2ccc(-c3ccc4c5ccccc5n(-c5ccccc5)c4c3)cc2)c([2H])c([2H])c1-c1cccc2cccc(-c3ccccc3)c12 ; [2H]c1c([2H])c(N(c2ccc(-c3ccccc3)cc2)c2ccc(-c3cccc4c5ccccc5n(-c5ccccc5)c34)cc2)c([2H])c([2H])c1-c1cccc2cccc(-c3ccccc3)c12]

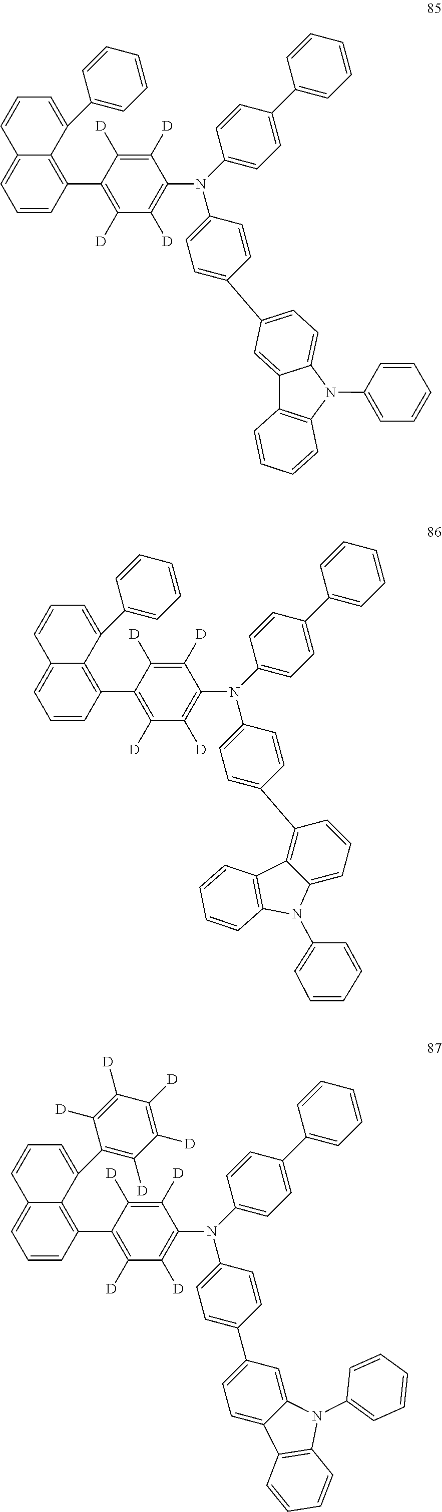 [SMILES: [2H]c1c([2H])c(N(c2ccc(-c3ccccc3)cc2)c2ccc(-c3ccc4c(c3)c3ccccc3n4-c3ccccc3)cc2)c([2H])c([2H])c1-c1cccc2cccc(-c3ccccc3)c12 ; [2H]c1c([2H])c(N(c2ccc(-c3ccccc3)cc2)c2ccc(-c3cccc4c3c3ccccc3n4-c3ccccc3)cc2)c([2H])c([2H])c1-c1cccc2cccc(-c3ccccc3)c12 ; [2H]c1c([2H])c([2H])c(-c2cccc3cccc(-c4c([2H])c([2H])c(N(c5ccc(-c6ccccc6)cc5)c5ccc(-c6ccc7c8ccccc8n(-c8ccccc8)c7c6)cc5)c([2H])c4[2H])c23)c([2H])c1[2H]]

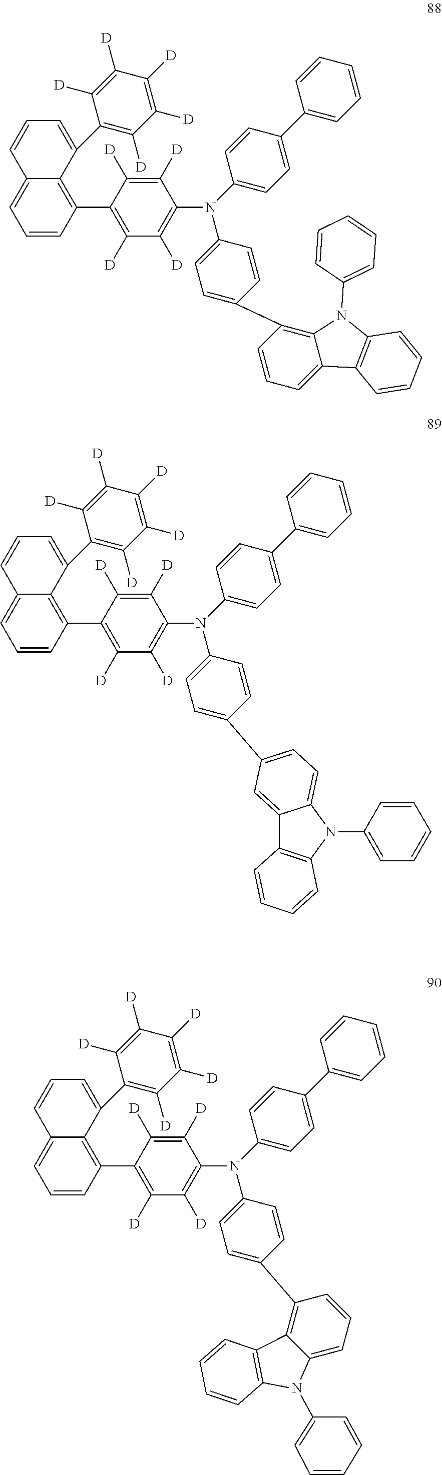 [SMILES: [2H]c1c([2H])c([2H])c(-c2cccc3cccc(-c4c([2H])c([2H])c(N(c5ccc(-c6ccccc6)cc5)c5ccc(-c6cccc7c8ccccc8n(-c8ccccc8)c67)cc5)c([2H])c4[2H])c23)c([2H])c1[2H] ; [2H]c1c([2H])c([2H])c(-c2cccc3cccc(-c4c([2H])c([2H])c(N(c5ccc(-c6ccccc6)cc5)c5ccc(-c6ccc7c(c6)c6ccccc6n7-c6ccccc6)cc5)c([2H])c4[2H])c23)c([2H])c1[2H] ; [2H]c1c([2H])c([2H])c(-c2cccc3cccc(-c4c([2H])c([2H])c(N(c5ccc(-c6ccccc6)cc5)c5ccc(-c6cccc7c6c6ccccc6n7-c6ccccc6)cc5)c([2H])c4[2H])c23)c([2H])c1[2H]]

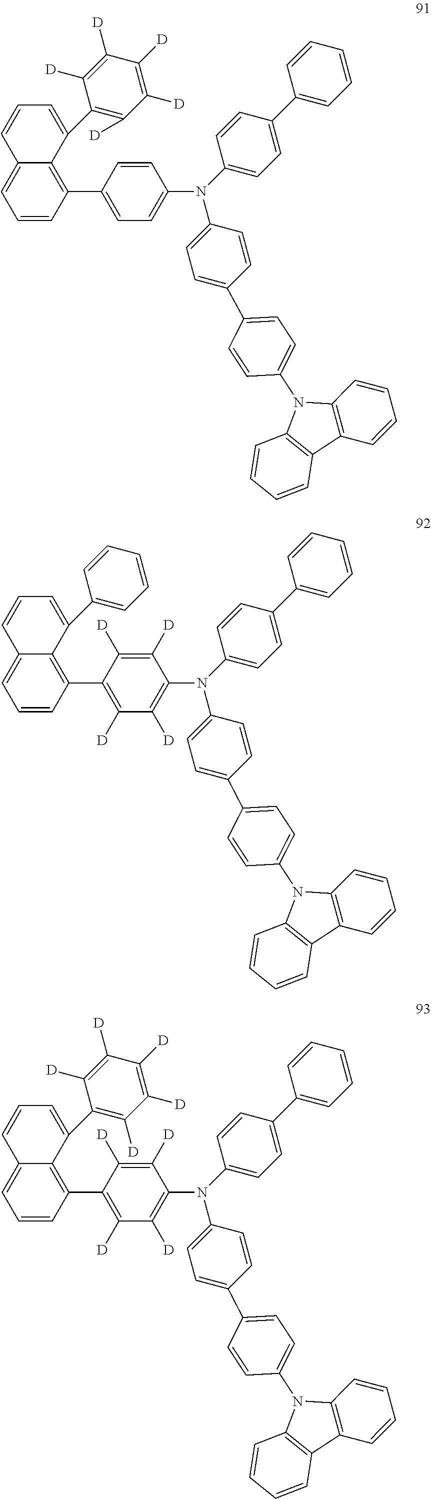

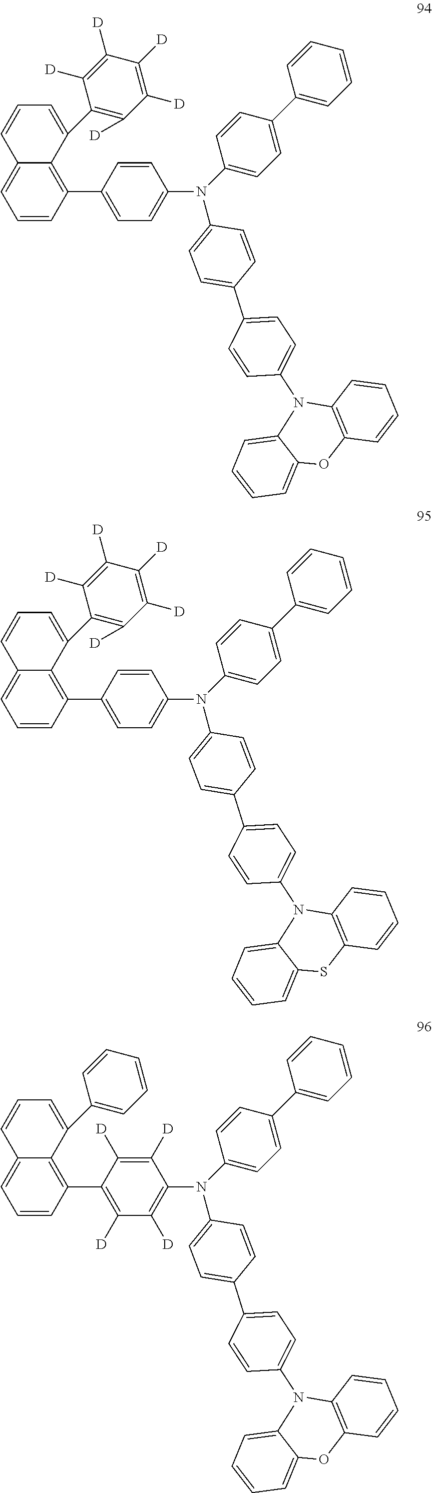

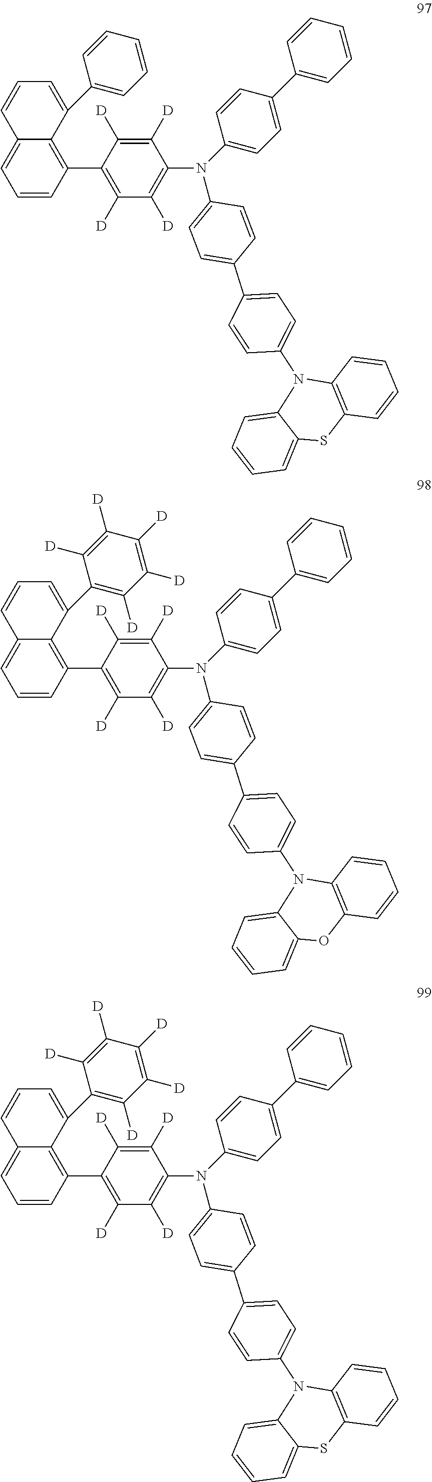 [SMILES: [2H]c1c([2H])c(N(c2ccc(-c3ccccc3)cc2)c2ccc(-c3ccc(N4c5ccccc5Sc5ccccc54)cc3)cc2)c([2H])c([2H])c1-c1cccc2cccc(-c3ccccc3)c12 ; [2H]c1c([2H])c([2H])c(-c2cccc3cccc(-c4c([2H])c([2H])c(N(c5ccc(-c6ccccc6)cc5)c5ccc(-c6ccc(N7c8ccccc8Oc8ccccc87)cc6)cc5)c([2H])c4[2H])c23)c([2H])c1[2H] ; [2H]c1c([2H])c([2H])c(-c2cccc3cccc(-c4c([2H])c([2H])c(N(c5ccc(-c6ccccc6)cc5)c5ccc(-c6ccc(N7c8ccccc8Sc8ccccc87)cc6)cc5)c([2H])c4[2H])c23)c([2H])c1[2H]]

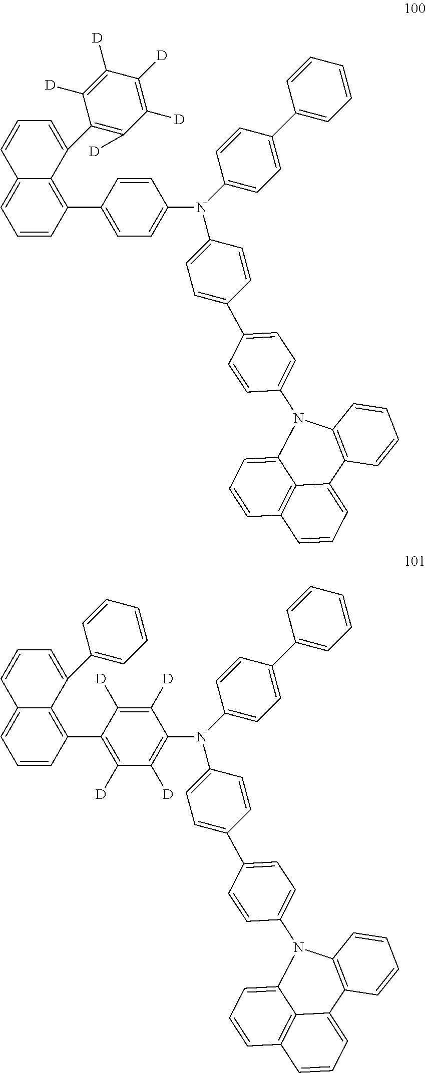

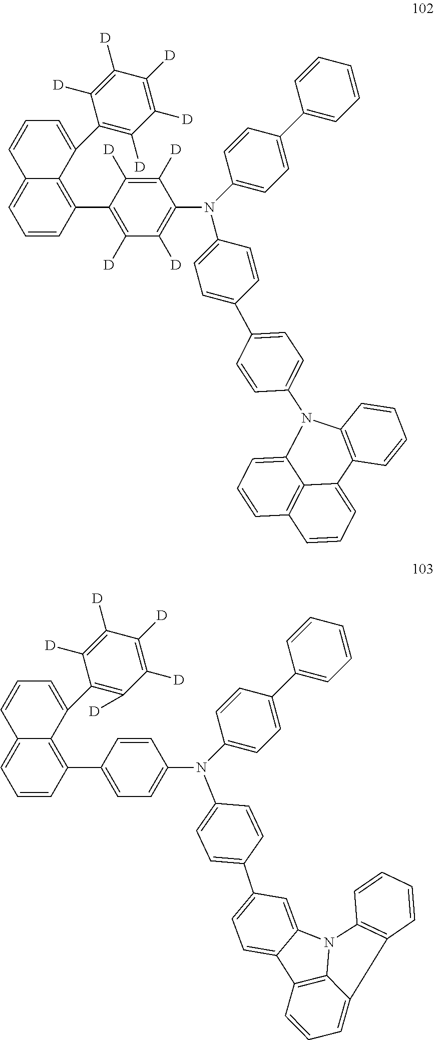 [SMILES: [2H]c1c([2H])c([2H])c(-c2cccc3cccc(-c4c([2H])c([2H])c(N(c5ccc(-c6ccccc6)cc5)c5ccc(-c6ccc(N7c8ccccc8-c8cccc9cccc7c89)cc6)cc5)c([2H])c4[2H])c23)c([2H])c1[2H] ; [2H]c1c([2H])c([2H])c(-c2cccc3cccc(-c4ccc(N(c5ccc(-c6ccccc6)cc5)c5ccc(-c6ccc7c8cccc9c%10ccccc%10n(c7c6)c98)cc5)cc4)c23)c([2H])c1[2H]]

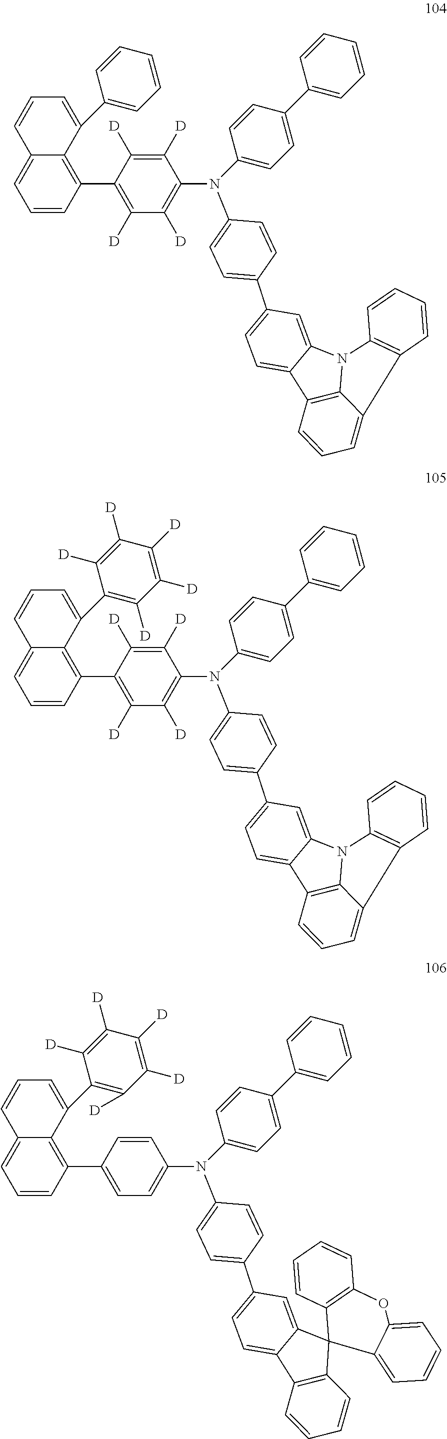 [SMILES: [2H]c1c([2H])c(N(c2ccc(-c3ccccc3)cc2)c2ccc(-c3ccc4c5cccc6c7ccccc7n(c4c3)c65)cc2)c([2H])c([2H])c1-c1cccc2cccc(-c3ccccc3)c12 ; [2H]c1c([2H])c([2H])c(-c2cccc3cccc(-c4c([2H])c([2H])c(N(c5ccc(-c6ccccc6)cc5)c5ccc(-c6ccc7c8cccc9c%10ccccc%10n(c7c6)c98)cc5)c([2H])c4[2H])c23)c([2H])c1[2H] ; [2H]c1c([2H])c([2H])c(-c2cccc3cccc(-c4ccc(N(c5ccc(-c6ccccc6)cc5)c5ccc(-c6ccc7c(c6)C6(c8ccccc8Oc8ccccc86)c6ccccc6-7)cc5)cc4)c23)c([2H])c1[2H]]

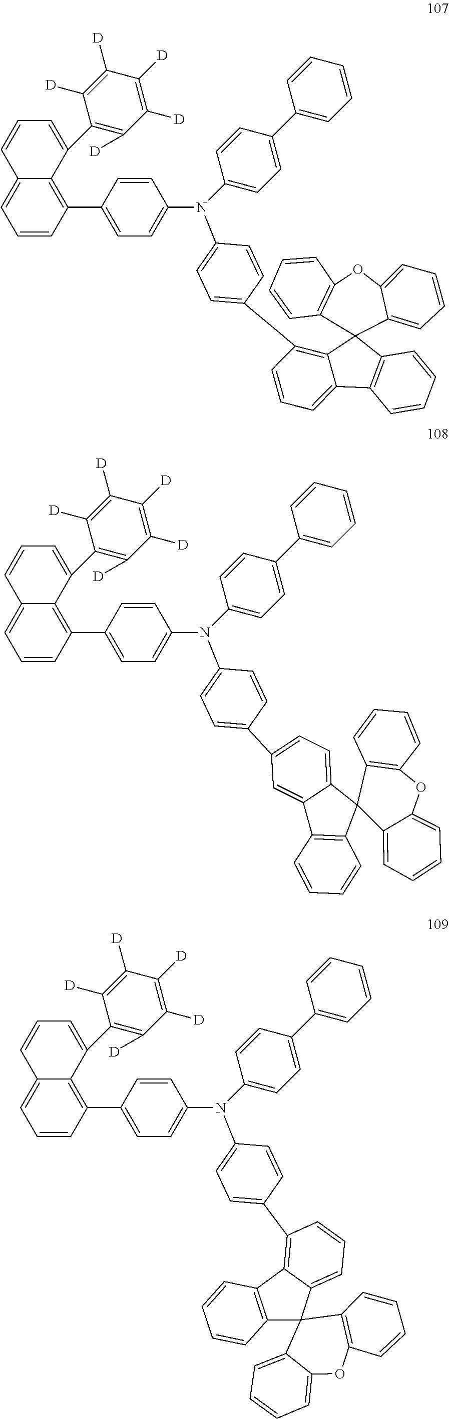 [SMILES: [2H]c1c([2H])c([2H])c(-c2cccc3cccc(-c4ccc(N(c5ccc(-c6ccccc6)cc5)c5ccc(-c6cccc7c6C6(c8ccccc8Oc8ccccc86)c6ccccc6-7)cc5)cc4)c23)c([2H])c1[2H] ; [2H]c1c([2H])c([2H])c(-c2cccc3cccc(-c4ccc(N(c5ccc(-c6ccccc6)cc5)c5ccc(-c6ccc7c(c6)-c6ccccc6C76c7ccccc7Oc7ccccc76)cc5)cc4)c23)c([2H])c1[2H] ; [2H]c1c([2H])c([2H])c(-c2cccc3cccc(-c4ccc(N(c5ccc(-c6ccccc6)cc5)c5ccc(-c6cccc7c6-c6ccccc6C76c7ccccc7Oc7ccccc76)cc5)cc4)c23)c([2H])c1[2H]]

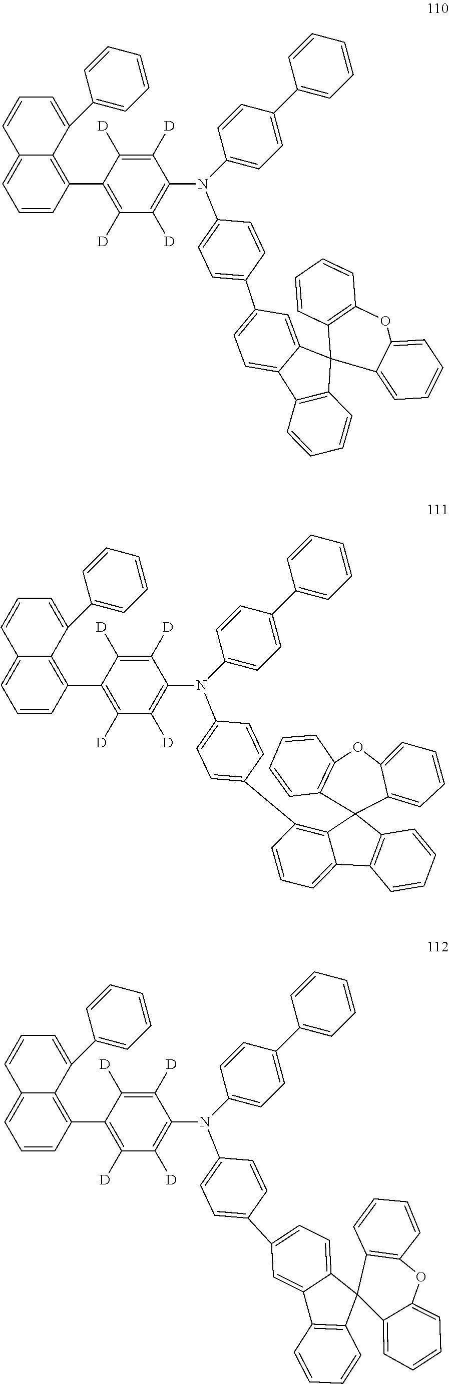 [SMILES: [2H]c1c([2H])c(N(c2ccc(-c3ccccc3)cc2)c2ccc(-c3ccc4c(c3)C3(c5ccccc5Oc5ccccc53)c3ccccc3-4)cc2)c([2H])c([2H])c1-c1cccc2cccc(-c3ccccc3)c12 ; [2H]c1c([2H])c(N(c2ccc(-c3ccccc3)cc2)c2ccc(-c3cccc4c3C3(c5ccccc5Oc5ccccc53)c3ccccc3-4)cc2)c([2H])c([2H])c1-c1cccc2cccc(-c3ccccc3)c12 ; [2H]c1c([2H])c(N(c2ccc(-c3ccccc3)cc2)c2ccc(-c3ccc4c(c3)-c3ccccc3C43c4ccccc4Oc4ccccc43)cc2)c([2H])c([2H])c1-c1cccc2cccc(-c3ccccc3)c12]

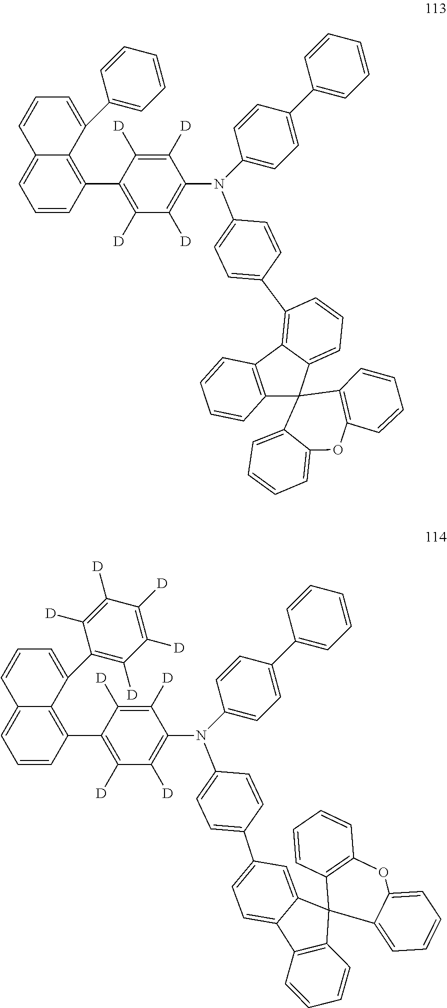 [SMILES: [2H]c1c([2H])c(N(c2ccc(-c3ccccc3)cc2)c2ccc(-c3cccc4c3-c3ccccc3C43c4ccccc4Oc4ccccc43)cc2)c([2H])c([2H])c1-c1cccc2cccc(-c3ccccc3)c12 ; [2H]c1c([2H])c([2H])c(-c2cccc3cccc(-c4c([2H])c([2H])c(N(c5ccc(-c6ccccc6)cc5)c5ccc(-c6ccc7c(c6)C6(c8ccccc8Oc8ccccc86)c6ccccc6-7)cc5)c([2H])c4[2H])c23)c([2H])c1[2H]]

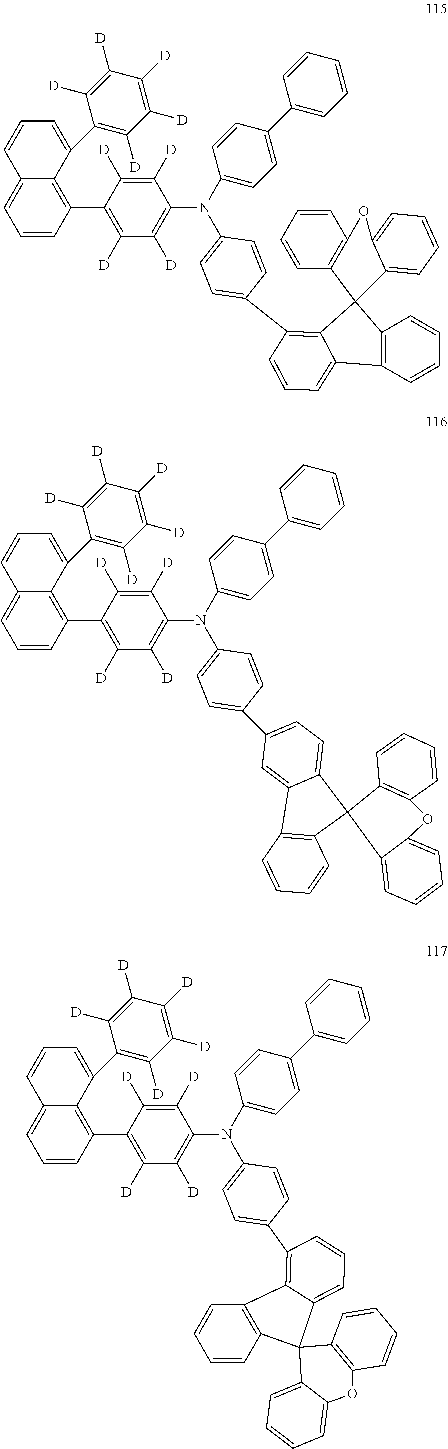 [SMILES: [2H]c1c([2H])c([2H])c(-c2cccc3cccc(-c4c([2H])c([2H])c(N(c5ccc(-c6ccccc6)cc5)c5ccc(-c6cccc7c6C6(c8ccccc8Oc8ccccc86)c6ccccc6-7)cc5)c([2H])c4[2H])c23)c([2H])c1[2H] ; [2H]c1c([2H])c([2H])c(-c2cccc3cccc(-c4c([2H])c([2H])c(N(c5ccc(-c6ccccc6)cc5)c5ccc(-c6ccc7c(c6)-c6ccccc6C76c7ccccc7Oc7ccccc76)cc5)c([2H])c4[2H])c23)c([2H])c1[2H] ; [2H]c1c([2H])c([2H])c(-c2cccc3cccc(-c4c([2H])c([2H])c(N(c5ccc(-c6ccccc6)cc5)c5ccc(-c6cccc7c6-c6ccccc6C76c7ccccc7Oc7ccccc76)cc5)c([2H])c4[2H])c23)c([2H])c1[2H]]

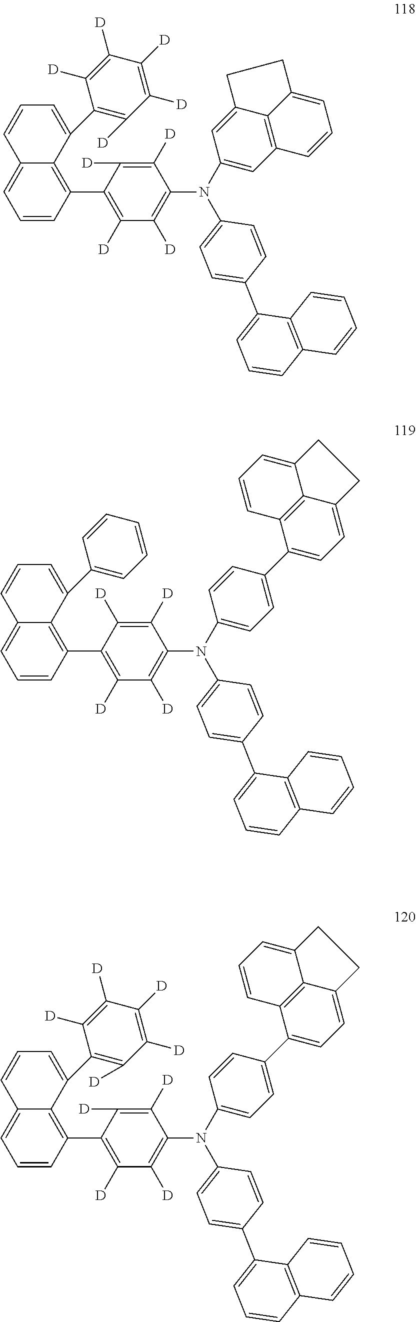 [SMILES: [2H]c1c([2H])c([2H])c(-c2cccc3cccc(-c4c([2H])c([2H])c(N(c5ccc(-c6cccc7ccccc67)cc5)c5cc6c7c(cccc7c5)CC6)c([2H])c4[2H])c23)c([2H])c1[2H] ; [2H]c1c([2H])c(N(c2ccc(-c3cccc4ccccc34)cc2)c2ccc(-c3ccc4c5c(cccc35)CC4)cc2)c([2H])c([2H])c1-c1cccc2cccc(-c3ccccc3)c12 ; [2H]c1c([2H])c([2H])c(-c2cccc3cccc(-c4c([2H])c([2H])c(N(c5ccc(-c6cccc7ccccc67)cc5)c5ccc(-c6ccc7c8c(cccc68)CC7)cc5)c([2H])c4[2H])c23)c([2H])c1[2H]]

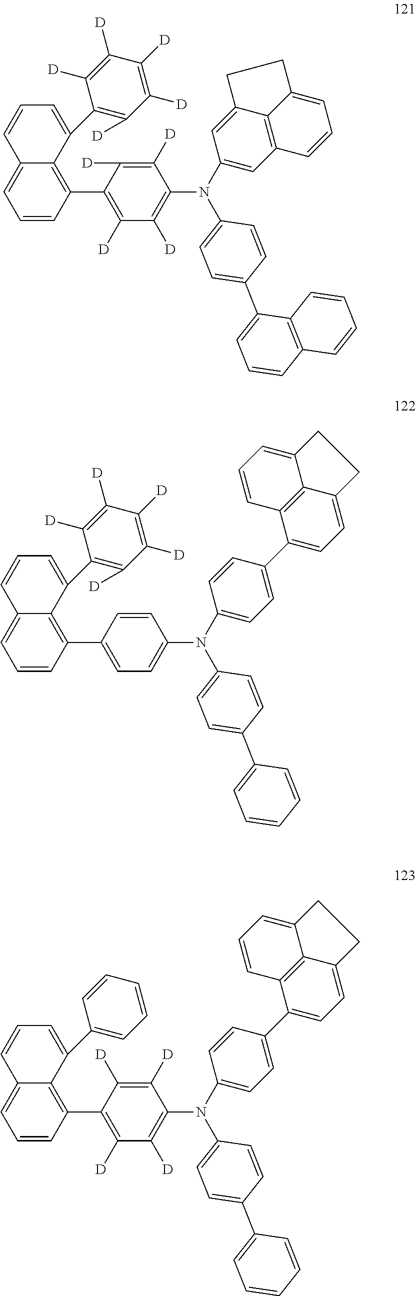 [SMILES: [2H]c1c([2H])c([2H])c(-c2cccc3cccc(-c4c([2H])c([2H])c(N(c5ccc(-c6cccc7ccccc67)cc5)c5cc6c7c(cccc7c5)CC6)c([2H])c4[2H])c23)c([2H])c1[2H] ; [2H]c1c([2H])c([2H])c(-c2cccc3cccc(-c4ccc(N(c5ccc(-c6ccccc6)cc5)c5ccc(-c6ccc7c8c(cccc68)CC7)cc5)cc4)c23)c([2H])c1[2H] ; [2H]c1c([2H])c(N(c2ccc(-c3ccccc3)cc2)c2ccc(-c3ccc4c5c(cccc35)CC4)cc2)c([2H])c([2H])c1-c1cccc2cccc(-c3ccccc3)c12]

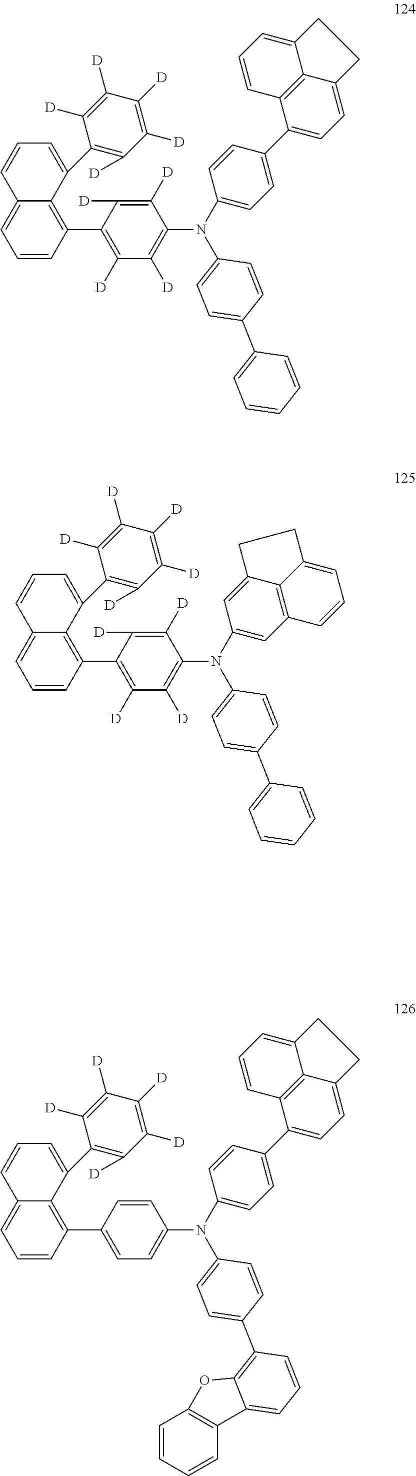 [SMILES: [2H]c1c([2H])c([2H])c(-c2cccc3cccc(-c4c([2H])c([2H])c(N(c5ccc(-c6ccccc6)cc5)c5ccc(-c6ccc7c8c(cccc68)CC7)cc5)c([2H])c4[2H])c23)c([2H])c1[2H] ; [2H]c1c([2H])c([2H])c(-c2cccc3cccc(-c4c([2H])c([2H])c(N(c5ccc(-c6ccccc6)cc5)c5cc6c7c(cccc7c5)CC6)c([2H])c4[2H])c23)c([2H])c1[2H] ; [2H]c1c([2H])c([2H])c(-c2cccc3cccc(-c4ccc(N(c5ccc(-c6ccc7c8c(cccc68)CC7)cc5)c5ccc(-c6cccc7c6oc6ccccc67)cc5)cc4)c23)c([2H])c1[2H]]

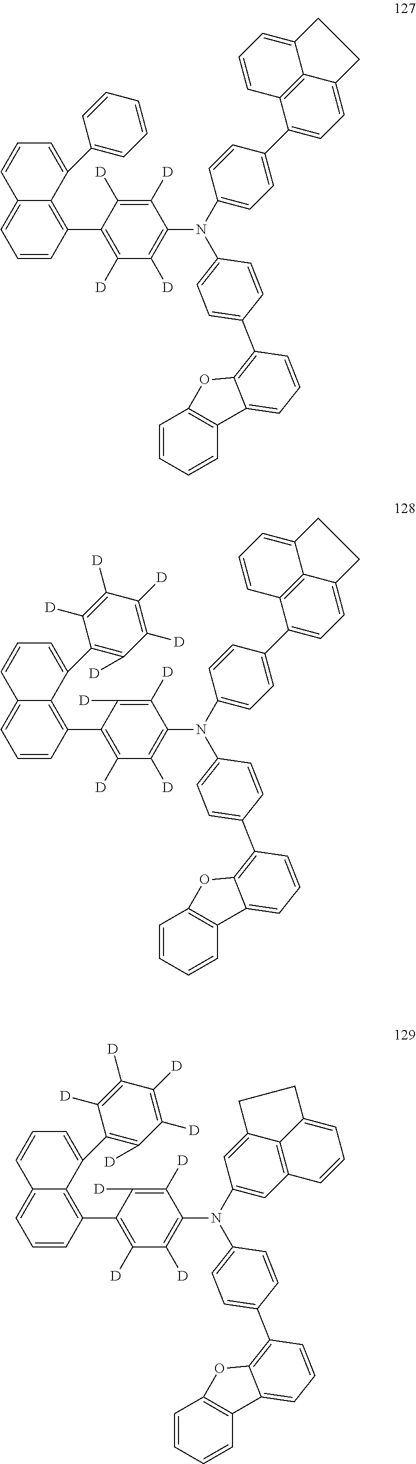 [SMILES: [2H]c1c([2H])c(N(c2ccc(-c3ccc4c5c(cccc35)CC4)cc2)c2ccc(-c3cccc4c3oc3ccccc34)cc2)c([2H])c([2H])c1-c1cccc2cccc(-c3ccccc3)c12 ; [2H]c1c([2H])c([2H])c(-c2cccc3cccc(-c4c([2H])c([2H])c(N(c5ccc(-c6ccc7c8c(cccc68)CC7)cc5)c5ccc(-c6cccc7c6oc6ccccc67)cc5)c([2H])c4[2H])c23)c([2H])c1[2H] ; [2H]c1c([2H])c([2H])c(-c2cccc3cccc(-c4c([2H])c([2H])c(N(c5ccc(-c6cccc7c6oc6ccccc67)cc5)c5cc6c7c(cccc7c5)CC6)c([2H])c4[2H])c23)c([2H])c1[2H]]

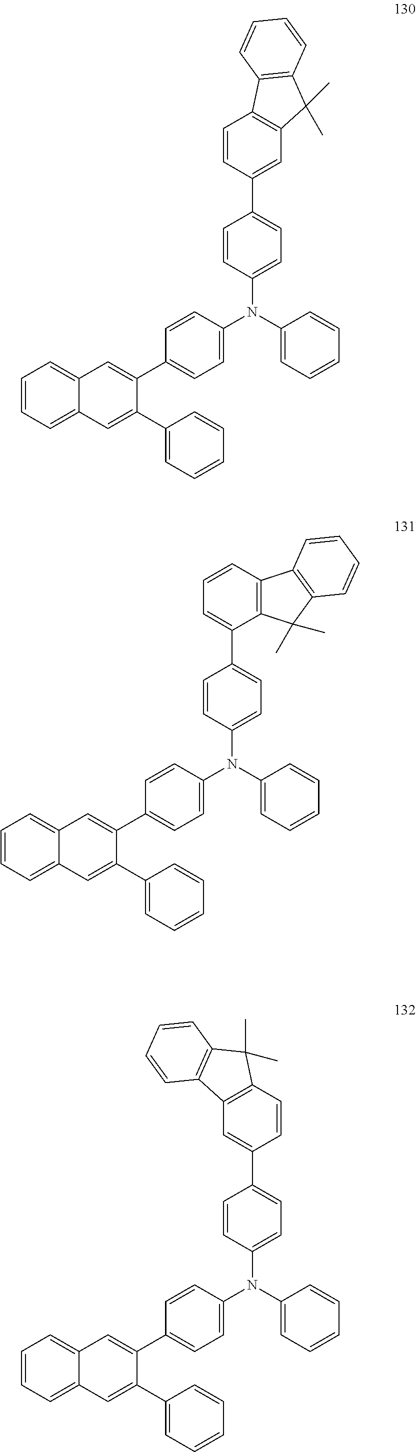 [SMILES: CC1(C)c2ccccc2-c2ccc(-c3ccc(N(c4ccccc4)c4ccc(-c5cc6ccccc6cc5-c5ccccc5)cc4)cc3)cc21 ; CC1(C)c2ccccc2-c2cccc(-c3ccc(N(c4ccccc4)c4ccc(-c5cc6ccccc6cc5-c5ccccc5)cc4)cc3)c21 ; CC1(C)c2ccccc2-c2cc(-c3ccc(N(c4ccccc4)c4ccc(-c5cc6ccccc6cc5-c5ccccc5)cc4)cc3)ccc21]

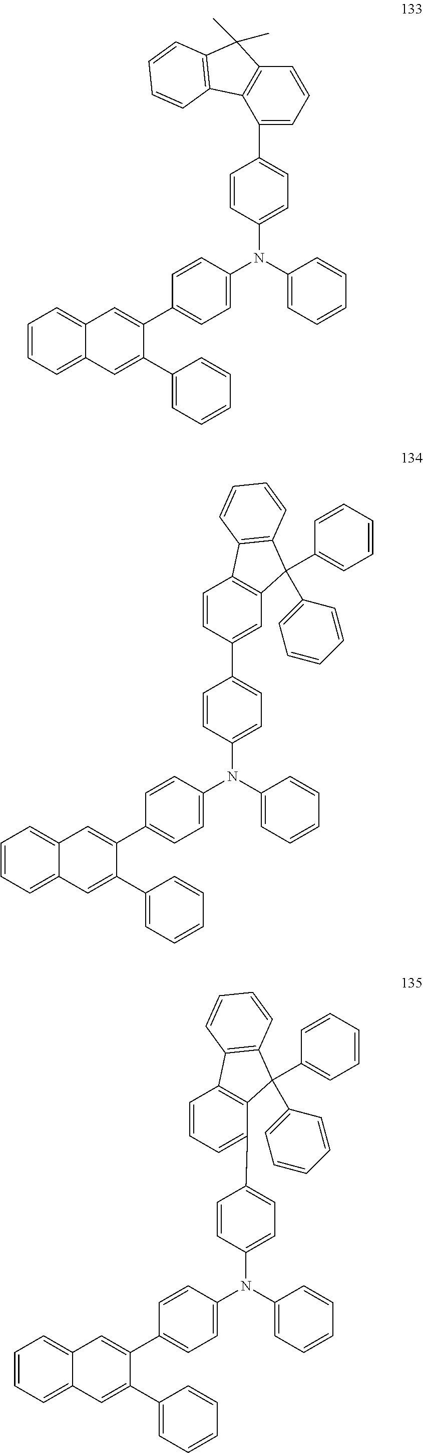 [SMILES: CC1(C)c2ccccc2-c2c(-c3ccc(N(c4ccccc4)c4ccc(-c5cc6ccccc6cc5-c5ccccc5)cc4)cc3)cccc21 ; c1ccc(-c2cc3ccccc3cc2-c2ccc(N(c3ccccc3)c3ccc(-c4ccc5c(c4)C(c4ccccc4)(c4ccccc4)c4ccccc4-5)cc3)cc2)cc1 ; c1ccc(-c2cc3ccccc3cc2-c2ccc(N(c3ccccc3)c3ccc(-c4cccc5c4C(c4ccccc4)(c4ccccc4)c4ccccc4-5)cc3)cc2)cc1]

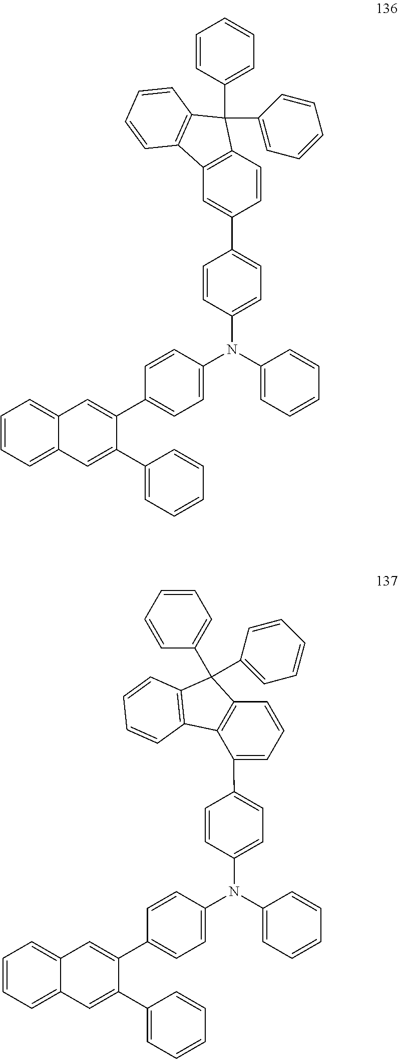 [SMILES: c1ccc(-c2cc3ccccc3cc2-c2ccc(N(c3ccccc3)c3ccc(-c4ccc5c(c4)-c4ccccc4C5(c4ccccc4)c4ccccc4)cc3)cc2)cc1 ; c1ccc(-c2cc3ccccc3cc2-c2ccc(N(c3ccccc3)c3ccc(-c4cccc5c4-c4ccccc4C5(c4ccccc4)c4ccccc4)cc3)cc2)cc1]

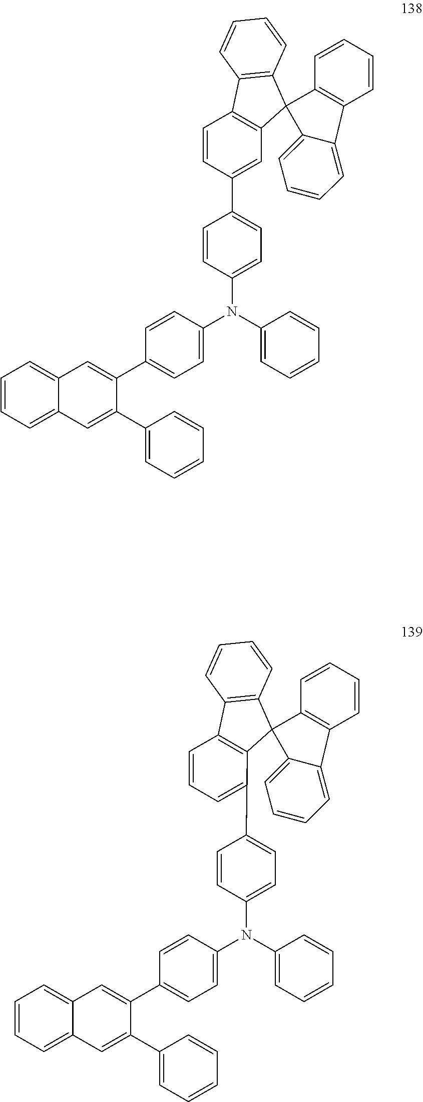 [SMILES: c1ccc(-c2cc3ccccc3cc2-c2ccc(N(c3ccccc3)c3ccc(-c4ccc5c(c4)C4(c6ccccc6-c6ccccc64)c4ccccc4-5)cc3)cc2)cc1 ; c1ccc(-c2cc3ccccc3cc2-c2ccc(N(c3ccccc3)c3ccc(-c4cccc5c4C4(c6ccccc6-c6ccccc64)c4ccccc4-5)cc3)cc2)cc1]

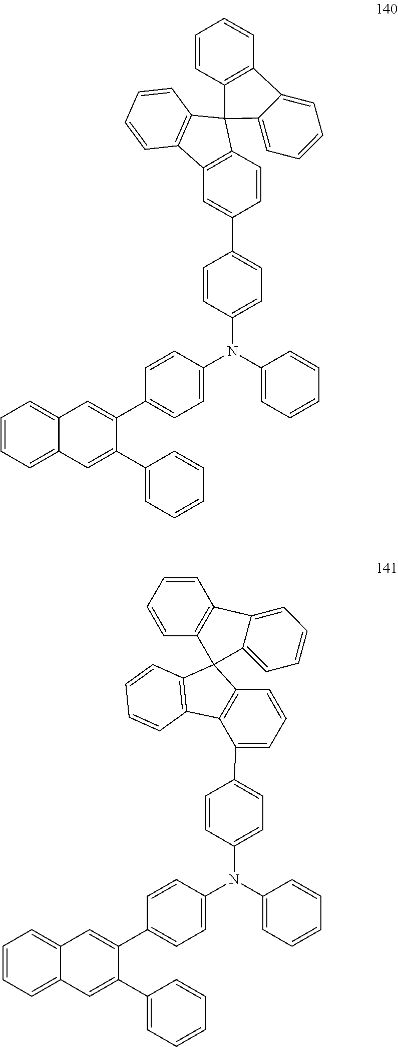 [SMILES: c1ccc(-c2cc3ccccc3cc2-c2ccc(N(c3ccccc3)c3ccc(-c4ccc5c(c4)-c4ccccc4C54c5ccccc5-c5ccccc54)cc3)cc2)cc1 ; c1ccc(-c2cc3ccccc3cc2-c2ccc(N(c3ccccc3)c3ccc(-c4cccc5c4-c4ccccc4C54c5ccccc5-c5ccccc54)cc3)cc2)cc1]

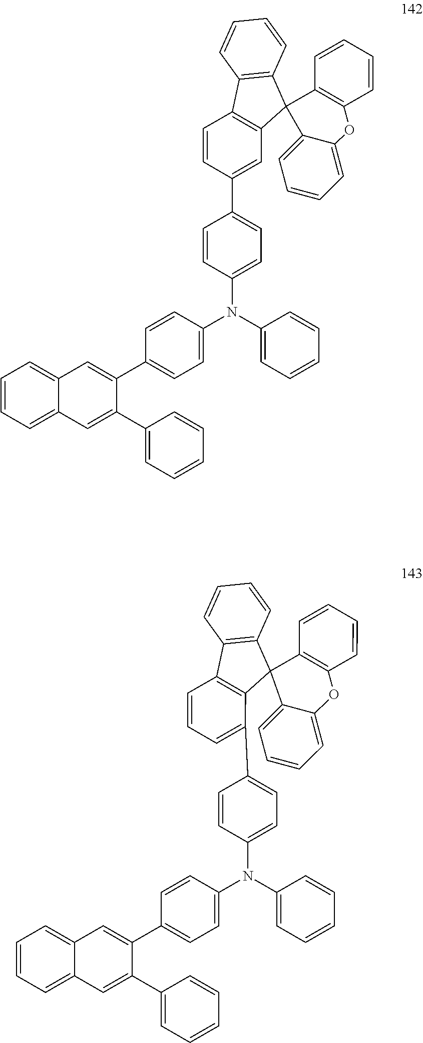 [SMILES: c1ccc(-c2cc3ccccc3cc2-c2ccc(N(c3ccccc3)c3ccc(-c4ccc5c(c4)C4(c6ccccc6Oc6ccccc64)c4ccccc4-5)cc3)cc2)cc1 ; c1ccc(-c2cc3ccccc3cc2-c2ccc(N(c3ccccc3)c3ccc(-c4cccc5c4C4(c6ccccc6Oc6ccccc64)c4ccccc4-5)cc3)cc2)cc1]

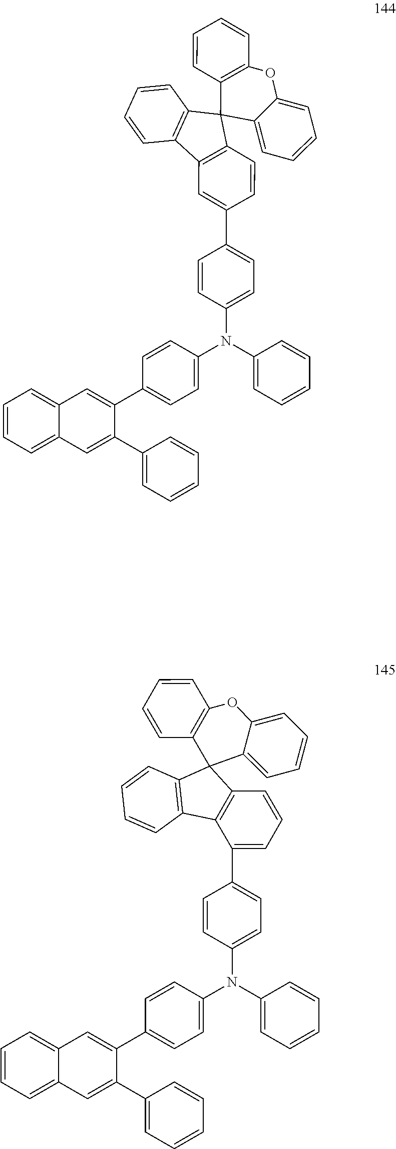 [SMILES: c1ccc(-c2cc3ccccc3cc2-c2ccc(N(c3ccccc3)c3ccc(-c4ccc5c(c4)-c4ccccc4C54c5ccccc5Oc5ccccc54)cc3)cc2)cc1 ; c1ccc(-c2cc3ccccc3cc2-c2ccc(N(c3ccccc3)c3ccc(-c4cccc5c4-c4ccccc4C54c5ccccc5Oc5ccccc54)cc3)cc2)cc1]

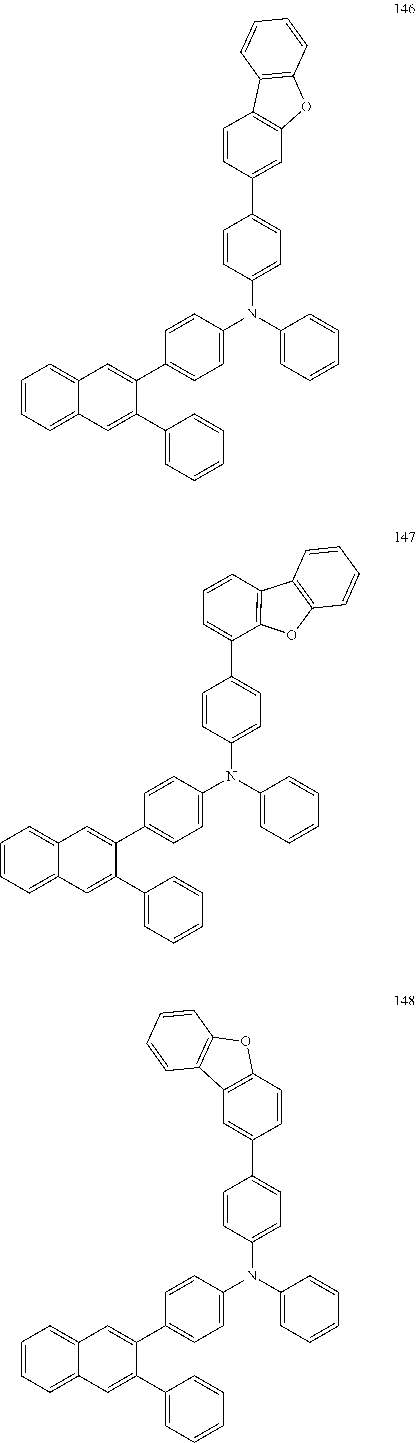 [SMILES: c1ccc(-c2cc3ccccc3cc2-c2ccc(N(c3ccccc3)c3ccc(-c4ccc5c(c4)oc4ccccc45)cc3)cc2)cc1 ; c1ccc(-c2cc3ccccc3cc2-c2ccc(N(c3ccccc3)c3ccc(-c4cccc5c4oc4ccccc45)cc3)cc2)cc1 ; c1ccc(-c2cc3ccccc3cc2-c2ccc(N(c3ccccc3)c3ccc(-c4ccc5oc6ccccc6c5c4)cc3)cc2)cc1]

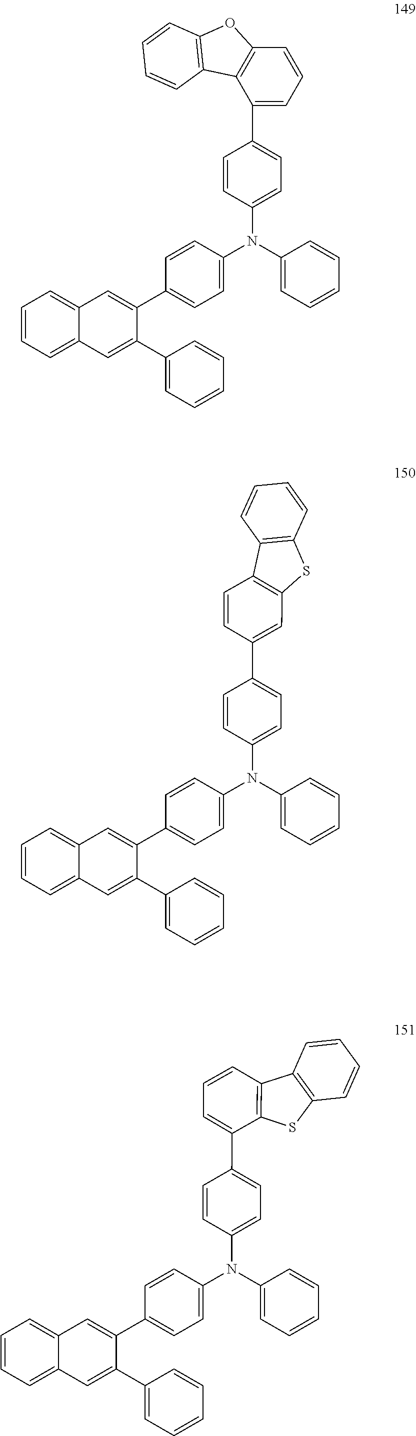 [SMILES: c1ccc(-c2cc3ccccc3cc2-c2ccc(N(c3ccccc3)c3ccc(-c4cccc5oc6ccccc6c45)cc3)cc2)cc1 ; c1ccc(-c2cc3ccccc3cc2-c2ccc(N(c3ccccc3)c3ccc(-c4ccc5c(c4)sc4ccccc45)cc3)cc2)cc1 ; c1ccc(-c2cc3ccccc3cc2-c2ccc(N(c3ccccc3)c3ccc(-c4cccc5c4sc4ccccc45)cc3)cc2)cc1]

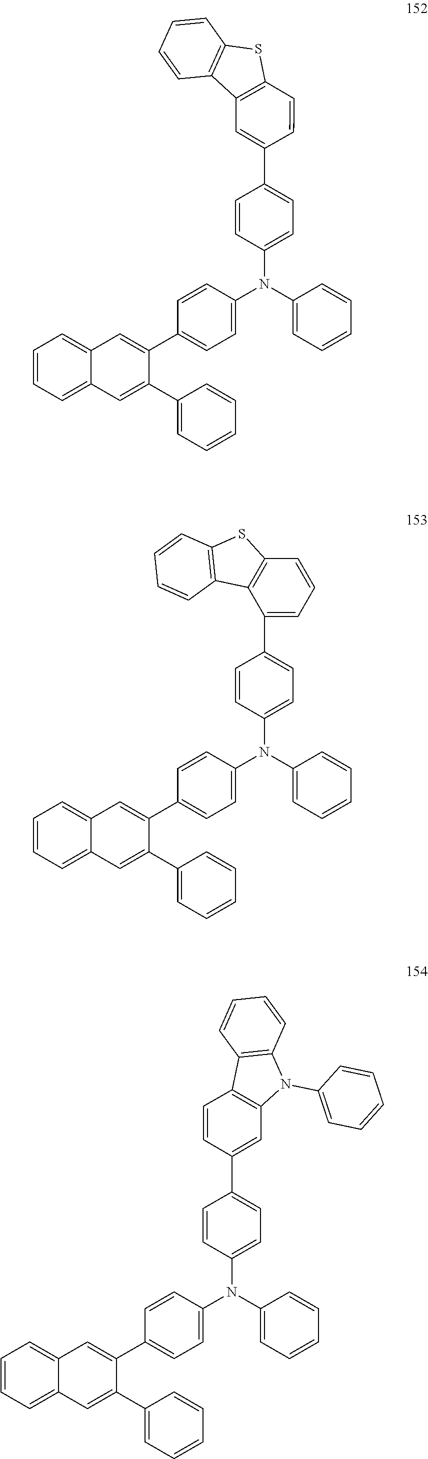 [SMILES: c1ccc(-c2cc3ccccc3cc2-c2ccc(N(c3ccccc3)c3ccc(-c4ccc5sc6ccccc6c5c4)cc3)cc2)cc1 ; c1ccc(-c2cc3ccccc3cc2-c2ccc(N(c3ccccc3)c3ccc(-c4cccc5sc6ccccc6c45)cc3)cc2)cc1 ; c1ccc(-c2cc3ccccc3cc2-c2ccc(N(c3ccccc3)c3ccc(-c4ccc5c6ccccc6n(-c6ccccc6)c5c4)cc3)cc2)cc1]

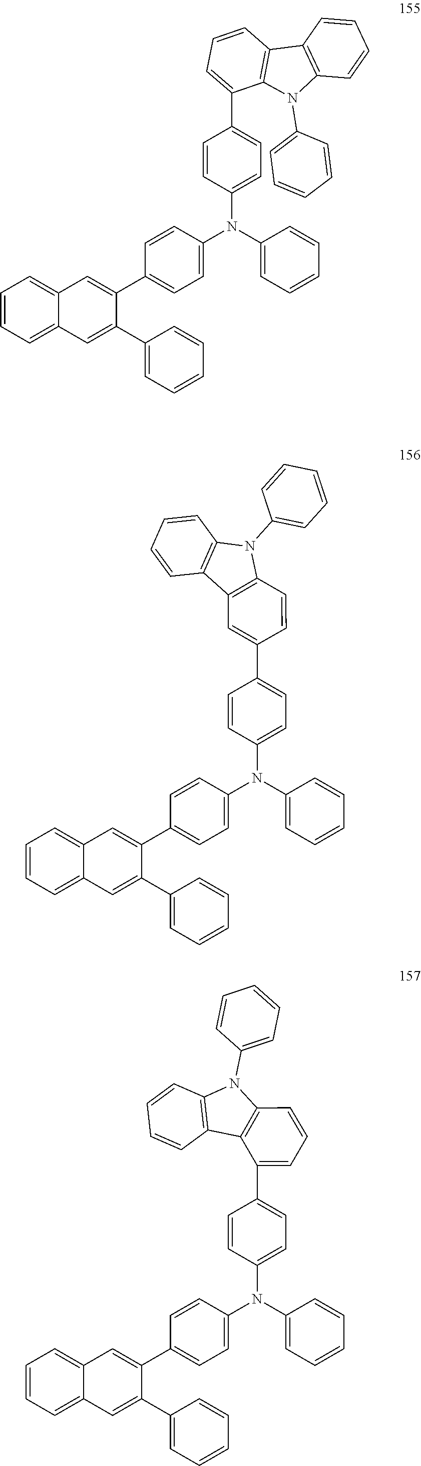 [SMILES: c1ccc(-c2cc3ccccc3cc2-c2ccc(N(c3ccccc3)c3ccc(-c4cccc5c6ccccc6n(-c6ccccc6)c45)cc3)cc2)cc1 ; c1ccc(-c2cc3ccccc3cc2-c2ccc(N(c3ccccc3)c3ccc(-c4ccc5c(c4)c4ccccc4n5-c4ccccc4)cc3)cc2)cc1 ; c1ccc(-c2cc3ccccc3cc2-c2ccc(N(c3ccccc3)c3ccc(-c4cccc5c4c4ccccc4n5-c4ccccc4)cc3)cc2)cc1]

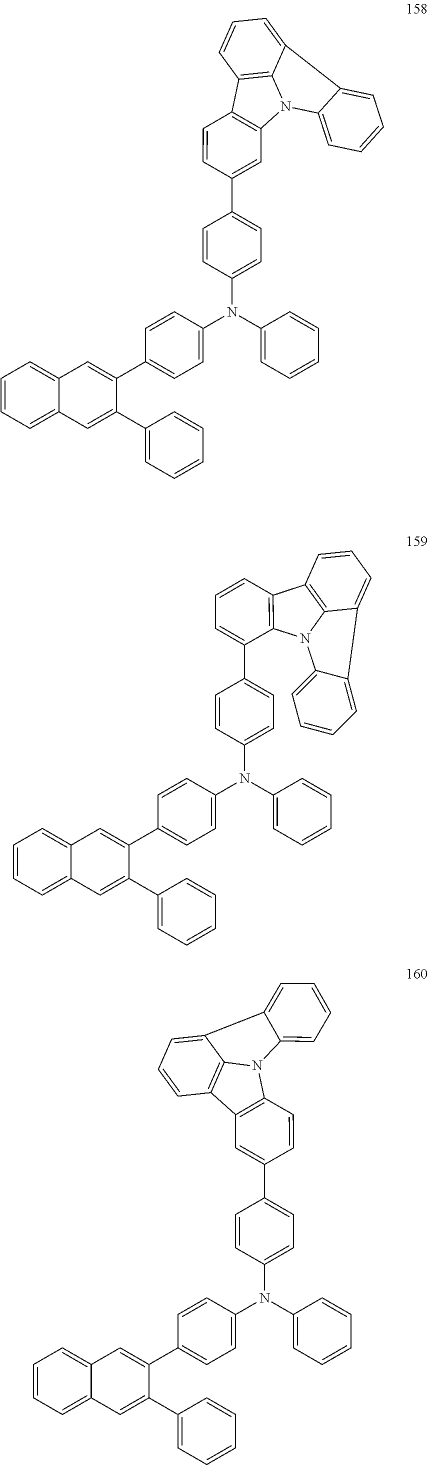 [SMILES: c1ccc(-c2cc3ccccc3cc2-c2ccc(N(c3ccccc3)c3ccc(-c4ccc5c6cccc7c8ccccc8n(c5c4)c76)cc3)cc2)cc1 ; c1ccc(-c2cc3ccccc3cc2-c2ccc(N(c3ccccc3)c3ccc(-c4cccc5c6cccc7c8ccccc8n(c45)c76)cc3)cc2)cc1 ; c1ccc(-c2cc3ccccc3cc2-c2ccc(N(c3ccccc3)c3ccc(-c4ccc5c(c4)c4cccc6c7ccccc7n5c64)cc3)cc2)cc1]

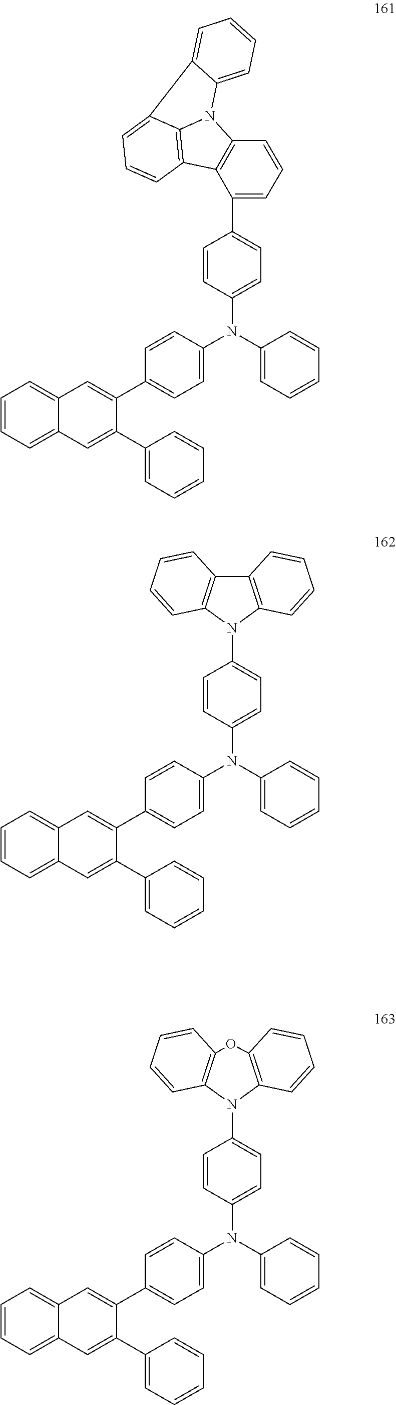 [SMILES: c1ccc(-c2cc3ccccc3cc2-c2ccc(N(c3ccccc3)c3ccc(-c4cccc5c4c4cccc6c7ccccc7n5c64)cc3)cc2)cc1 ; c1ccc(-c2cc3ccccc3cc2-c2ccc(N(c3ccccc3)c3ccc(-n4c5ccccc5c5ccccc54)cc3)cc2)cc1 ; c1ccc(-c2cc3ccccc3cc2-c2ccc(N(c3ccccc3)c3ccc(N4c5ccccc5Oc5ccccc54)cc3)cc2)cc1]

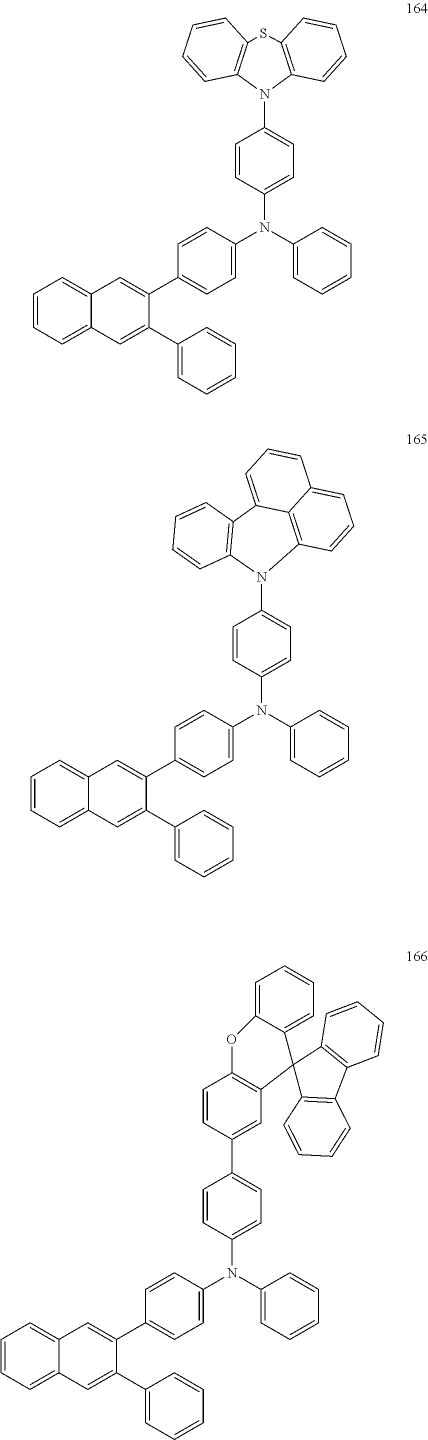 [SMILES: c1ccc(-c2cc3ccccc3cc2-c2ccc(N(c3ccccc3)c3ccc(N4c5ccccc5Sc5ccccc54)cc3)cc2)cc1 ; c1ccc(-c2cc3ccccc3cc2-c2ccc(N(c3ccccc3)c3ccc(N4c5ccccc5-c5cccc6cccc4c56)cc3)cc2)cc1 ; c1ccc(-c2cc3ccccc3cc2-c2ccc(N(c3ccccc3)c3ccc(-c4ccc5c(c4)C4(c6ccccc6O5)c5ccccc5-c5ccccc54)cc3)cc2)cc1]

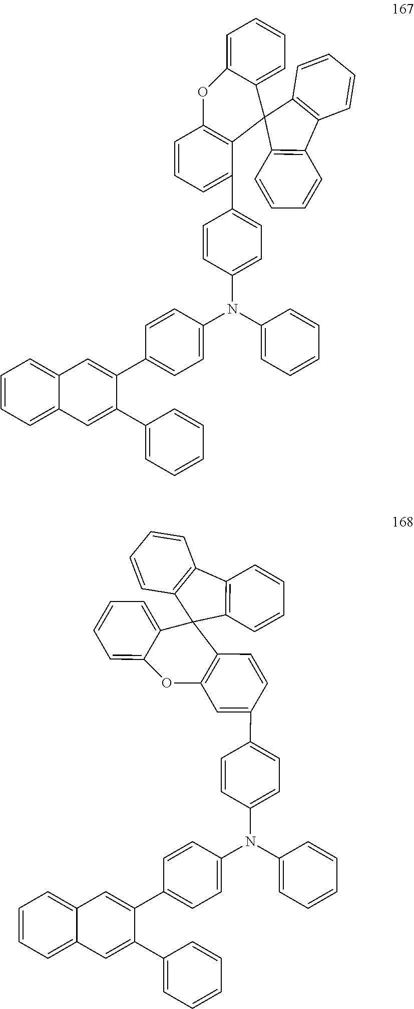 [SMILES: c1ccc(-c2cc3ccccc3cc2-c2ccc(N(c3ccccc3)c3ccc(-c4cccc5c4C4(c6ccccc6O5)c5ccccc5-c5ccccc54)cc3)cc2)cc1 ; c1ccc(-c2cc3ccccc3cc2-c2ccc(N(c3ccccc3)c3ccc(-c4ccc5c(c4)Oc4ccccc4C54c5ccccc5-c5ccccc54)cc3)cc2)cc1]

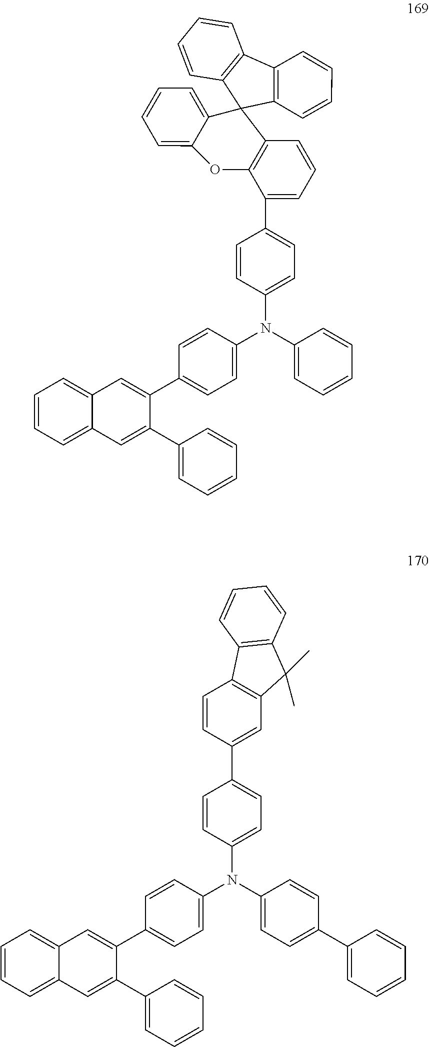 [SMILES: c1ccc(-c2cc3ccccc3cc2-c2ccc(N(c3ccccc3)c3ccc(-c4cccc5c4Oc4ccccc4C54c5ccccc5-c5ccccc54)cc3)cc2)cc1 ; CC1(C)c2ccccc2-c2ccc(-c3ccc(N(c4ccc(-c5ccccc5)cc4)c4ccc(-c5cc6ccccc6cc5-c5ccccc5)cc4)cc3)cc21]

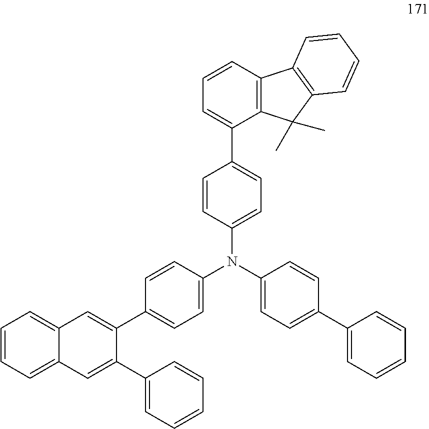 [SMILES: CC1(C)c2ccccc2-c2cccc(-c3ccc(N(c4ccc(-c5ccccc5)cc4)c4ccc(-c5cc6ccccc6cc5-c5ccccc5)cc4)cc3)c21]

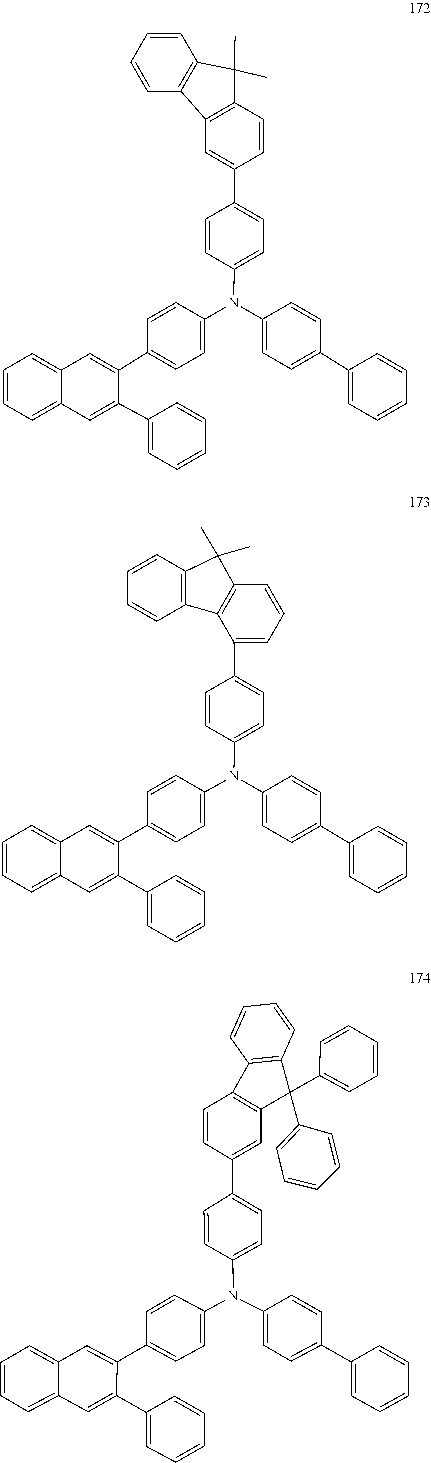 [SMILES: CC1(C)c2ccccc2-c2cc(-c3ccc(N(c4ccc(-c5ccccc5)cc4)c4ccc(-c5cc6ccccc6cc5-c5ccccc5)cc4)cc3)ccc21 ; CC1(C)c2ccccc2-c2c(-c3ccc(N(c4ccc(-c5ccccc5)cc4)c4ccc(-c5cc6ccccc6cc5-c5ccccc5)cc4)cc3)cccc21 ; c1ccc(-c2ccc(N(c3ccc(-c4ccc5c(c4)C(c4ccccc4)(c4ccccc4)c4ccccc4-5)cc3)c3ccc(-c4cc5ccccc5cc4-c4ccccc4)cc3)cc2)cc1]

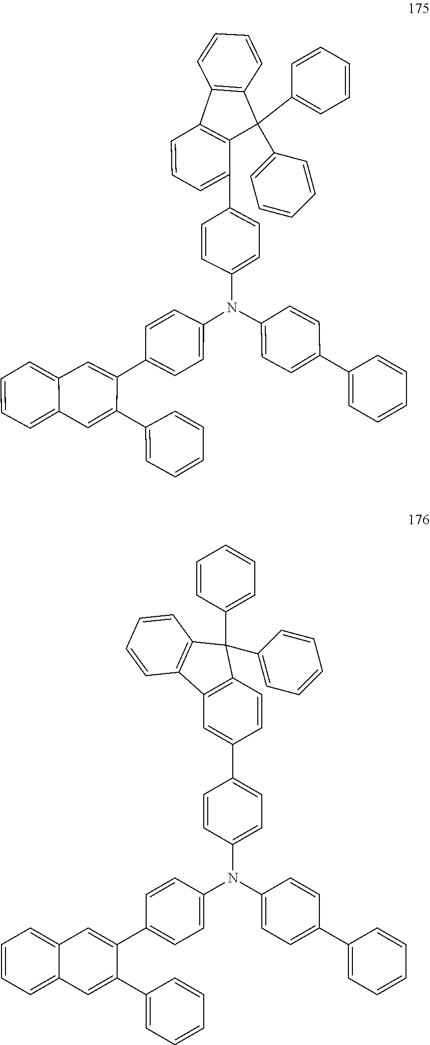 [SMILES: C1=CC2=C(CC(c3ccc(N(c4ccc(-c5ccccc5)cc4)c4ccc(-c5cc6ccccc6cc5-c5ccccc5)cc4)cc3)=C1)C(c1ccccc1)(c1ccccc1)c1ccccc12 ; c1ccc(-c2ccc(N(c3ccc(-c4ccc5c(c4)-c4ccccc4C5(c4ccccc4)c4ccccc4)cc3)c3ccc(-c4cc5ccccc5cc4-c4ccccc4)cc3)cc2)cc1]

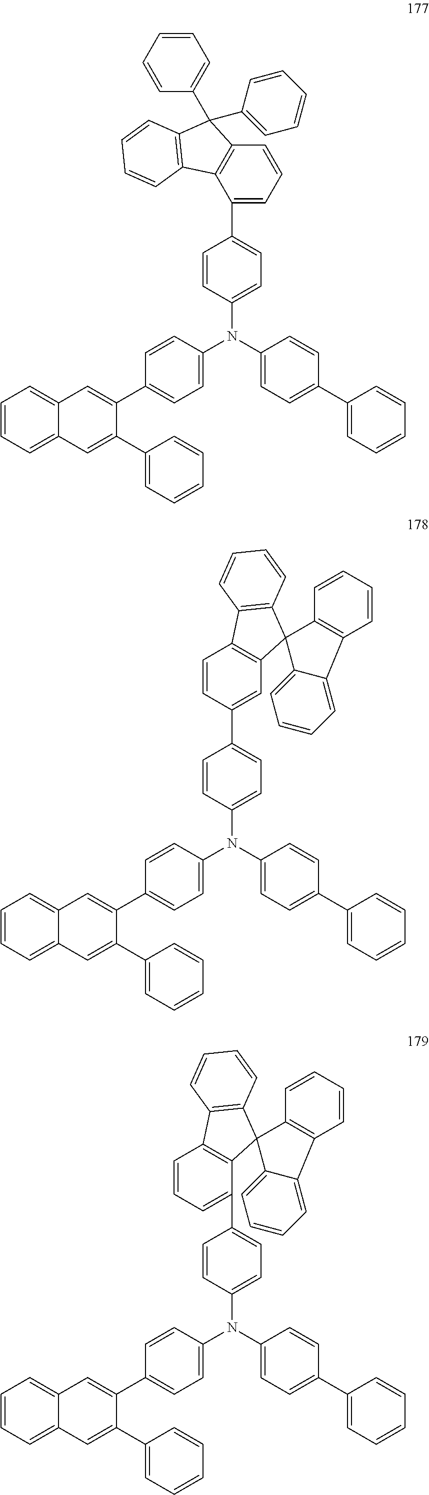 [SMILES: c1ccc(-c2ccc(N(c3ccc(-c4cc5ccccc5cc4-c4ccccc4)cc3)c3ccc(-c4cccc5c4-c4ccccc4C5(c4ccccc4)c4ccccc4)cc3)cc2)cc1 ; c1ccc(-c2ccc(N(c3ccc(-c4ccc5c(c4)C4(c6ccccc6-c6ccccc64)c4ccccc4-5)cc3)c3ccc(-c4cc5ccccc5cc4-c4ccccc4)cc3)cc2)cc1 ; c1ccc(-c2ccc(N(c3ccc(-c4cc5ccccc5cc4-c4ccccc4)cc3)c3ccc(-c4cccc5c4C4(c6ccccc6-c6ccccc64)c4ccccc4-5)cc3)cc2)cc1]

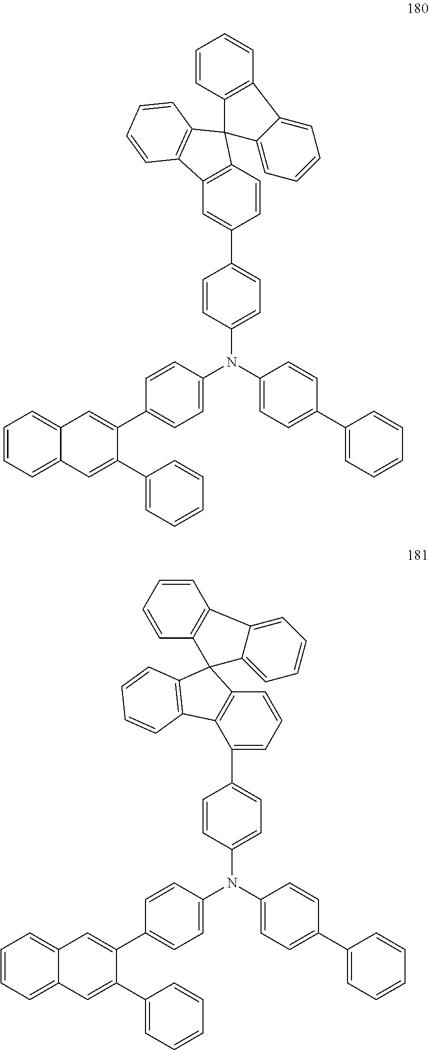 [SMILES: c1ccc(-c2ccc(N(c3ccc(-c4ccc5c(c4)-c4ccccc4C54c5ccccc5-c5ccccc54)cc3)c3ccc(-c4cc5ccccc5cc4-c4ccccc4)cc3)cc2)cc1 ; c1ccc(-c2ccc(N(c3ccc(-c4cc5ccccc5cc4-c4ccccc4)cc3)c3ccc(-c4cccc5c4-c4ccccc4C54c5ccccc5-c5ccccc54)cc3)cc2)cc1]

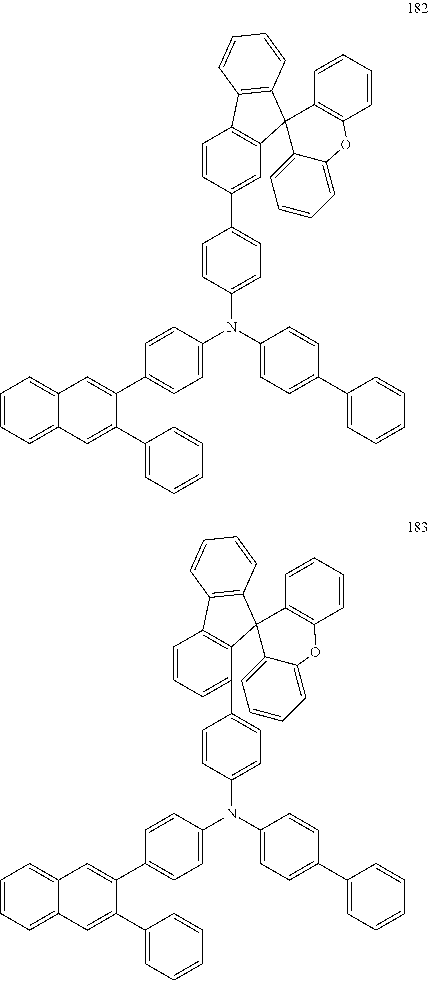 [SMILES: c1ccc(-c2ccc(N(c3ccc(-c4ccc5c(c4)C4(c6ccccc6Oc6ccccc64)c4ccccc4-5)cc3)c3ccc(-c4cc5ccccc5cc4-c4ccccc4)cc3)cc2)cc1 ; c1ccc(-c2ccc(N(c3ccc(-c4cc5ccccc5cc4-c4ccccc4)cc3)c3ccc(-c4cccc5c4C4(c6ccccc6Oc6ccccc64)c4ccccc4-5)cc3)cc2)cc1]

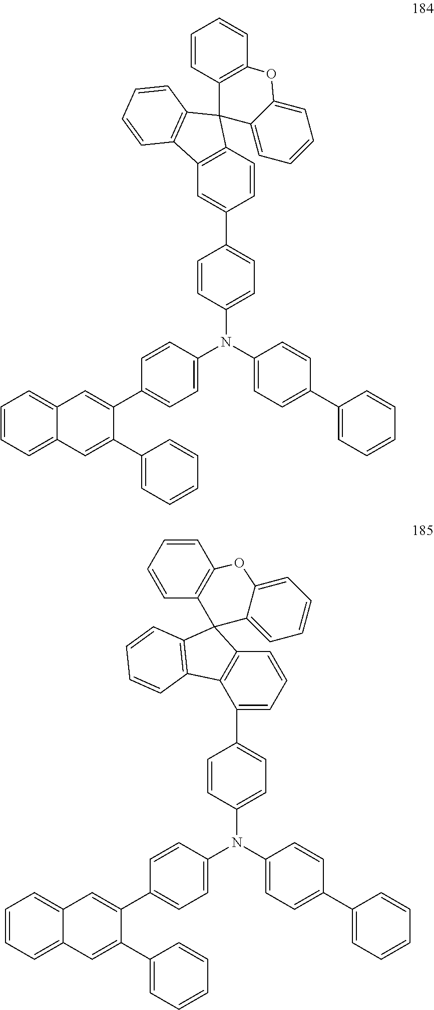

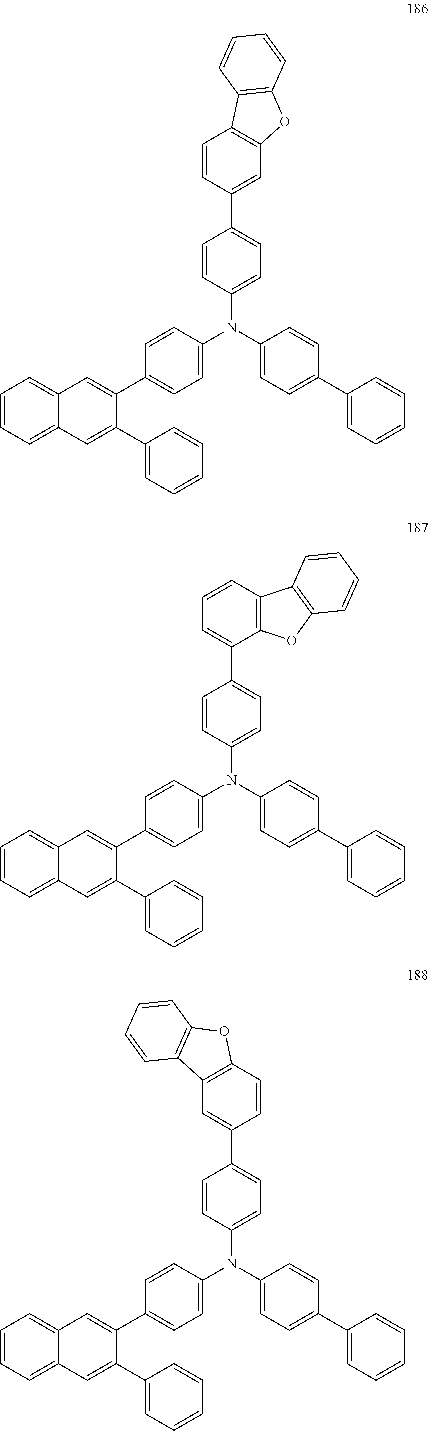 [SMILES: c1ccc(-c2ccc(N(c3ccc(-c4ccc5c(c4)oc4ccccc45)cc3)c3ccc(-c4cc5ccccc5cc4-c4ccccc4)cc3)cc2)cc1 ; c1ccc(-c2ccc(N(c3ccc(-c4cc5ccccc5cc4-c4ccccc4)cc3)c3ccc(-c4cccc5c4oc4ccccc45)cc3)cc2)cc1 ; c1ccc(-c2ccc(N(c3ccc(-c4ccc5oc6ccccc6c5c4)cc3)c3ccc(-c4cc5ccccc5cc4-c4ccccc4)cc3)cc2)cc1]

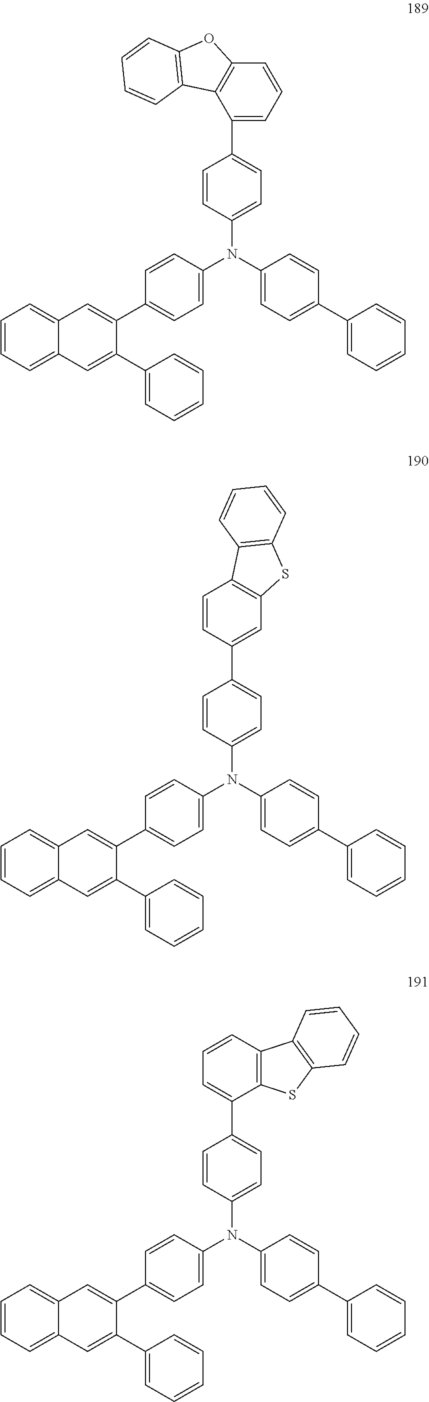 [SMILES: c1ccc(-c2ccc(N(c3ccc(-c4cc5ccccc5cc4-c4ccccc4)cc3)c3ccc(-c4cccc5oc6ccccc6c45)cc3)cc2)cc1 ; c1ccc(-c2ccc(N(c3ccc(-c4ccc5c(c4)sc4ccccc45)cc3)c3ccc(-c4cc5ccccc5cc4-c4ccccc4)cc3)cc2)cc1 ; c1ccc(-c2ccc(N(c3ccc(-c4cc5ccccc5cc4-c4ccccc4)cc3)c3ccc(-c4cccc5c4sc4ccccc45)cc3)cc2)cc1]

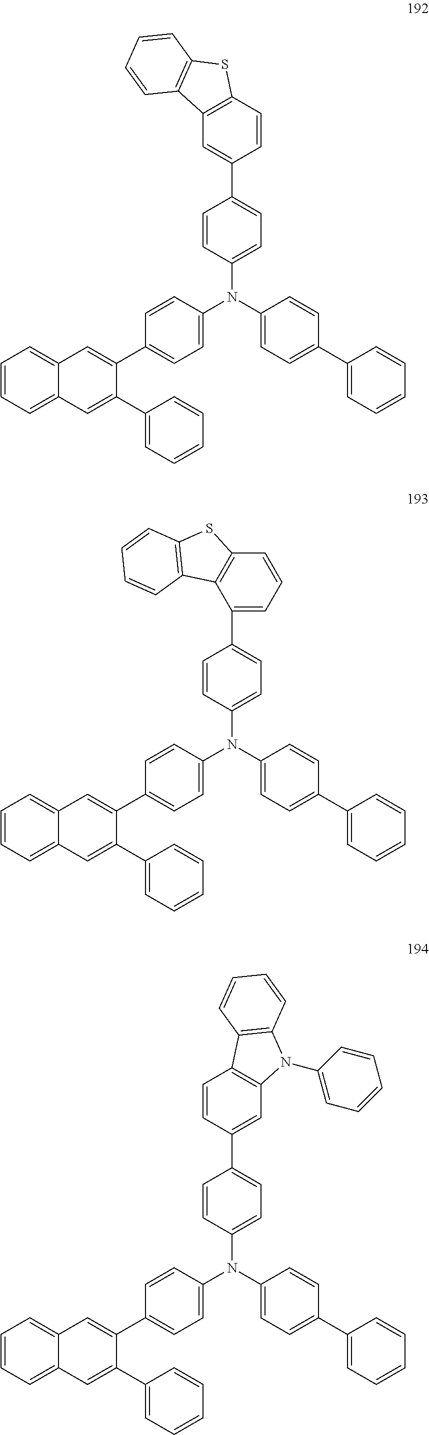 [SMILES: c1ccc(-c2ccc(N(c3ccc(-c4ccc5sc6ccccc6c5c4)cc3)c3ccc(-c4cc5ccccc5cc4-c4ccccc4)cc3)cc2)cc1 ; c1ccc(-c2ccc(N(c3ccc(-c4cc5ccccc5cc4-c4ccccc4)cc3)c3ccc(-c4cccc5sc6ccccc6c45)cc3)cc2)cc1 ; c1ccc(-c2ccc(N(c3ccc(-c4ccc5c6ccccc6n(-c6ccccc6)c5c4)cc3)c3ccc(-c4cc5ccccc5cc4-c4ccccc4)cc3)cc2)cc1]

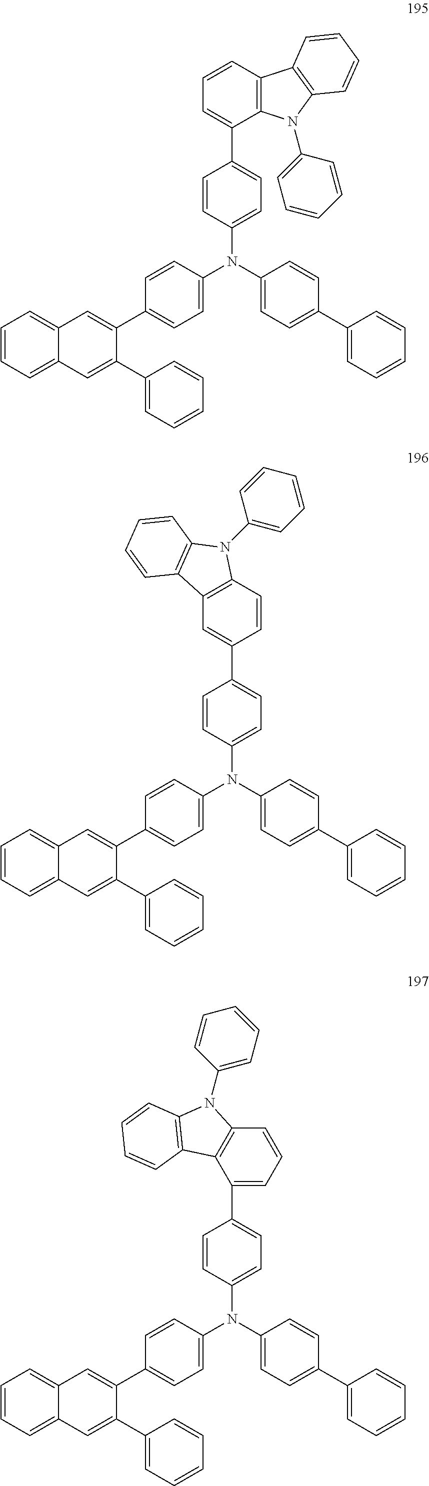 [SMILES: c1ccc(-c2ccc(N(c3ccc(-c4cc5ccccc5cc4-c4ccccc4)cc3)c3ccc(-c4cccc5c6ccccc6n(-c6ccccc6)c45)cc3)cc2)cc1 ; c1ccc(-c2ccc(N(c3ccc(-c4ccc5c(c4)c4ccccc4n5-c4ccccc4)cc3)c3ccc(-c4cc5ccccc5cc4-c4ccccc4)cc3)cc2)cc1 ; c1ccc(-c2ccc(N(c3ccc(-c4cc5ccccc5cc4-c4ccccc4)cc3)c3ccc(-c4cccc5c4c4ccccc4n5-c4ccccc4)cc3)cc2)cc1]

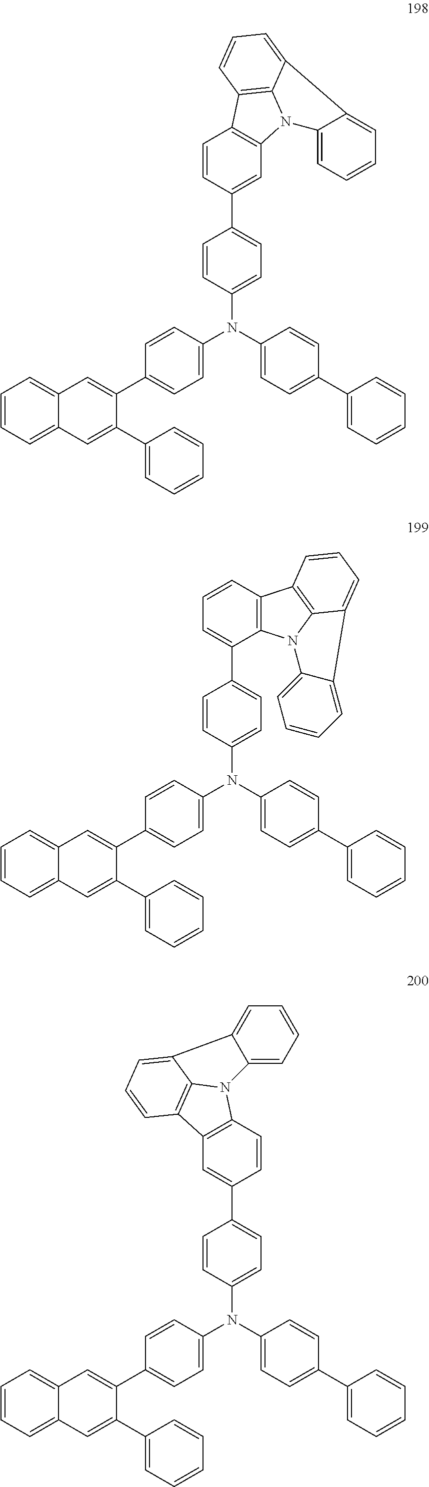 [SMILES: c1ccc(-c2ccc(N(c3ccc(-c4ccc5c6cccc7c8ccccc8n(c5c4)c76)cc3)c3ccc(-c4cc5ccccc5cc4-c4ccccc4)cc3)cc2)cc1 ; c1ccc(-c2ccc(N(c3ccc(-c4cc5ccccc5cc4-c4ccccc4)cc3)c3ccc(-c4cccc5c6cccc7c8ccccc8n(c45)c76)cc3)cc2)cc1 ; c1ccc(-c2ccc(N(c3ccc(-c4ccc5c(c4)c4cccc6c7ccccc7n5c64)cc3)c3ccc(-c4cc5ccccc5cc4-c4ccccc4)cc3)cc2)cc1]

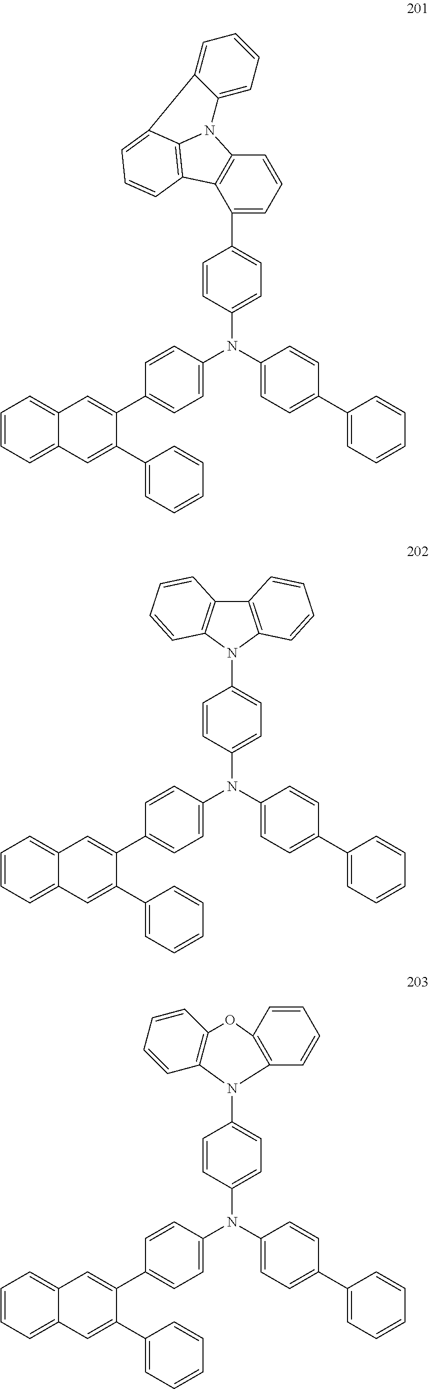 [SMILES: c1ccc(-c2ccc(N(c3ccc(-c4cc5ccccc5cc4-c4ccccc4)cc3)c3ccc(-c4cccc5c4c4cccc6c7ccccc7n5c64)cc3)cc2)cc1 ; c1ccc(-c2ccc(N(c3ccc(-c4cc5ccccc5cc4-c4ccccc4)cc3)c3ccc(-n4c5ccccc5c5ccccc54)cc3)cc2)cc1 ; c1ccc(-c2ccc(N(c3ccc(-c4cc5ccccc5cc4-c4ccccc4)cc3)c3ccc(N4c5ccccc5Oc5ccccc54)cc3)cc2)cc1]

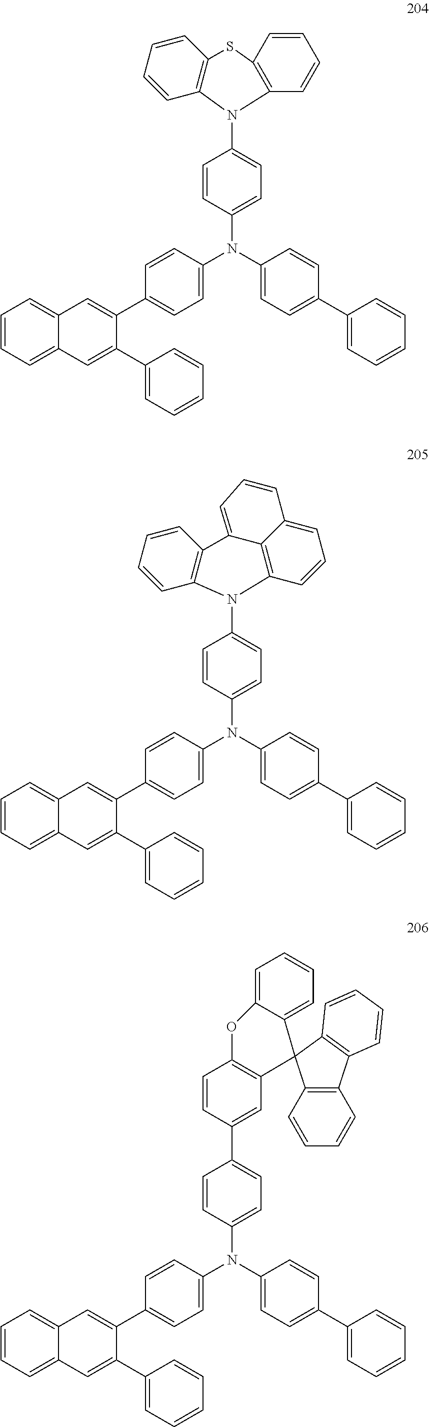 [SMILES: c1ccc(-c2ccc(N(c3ccc(-c4cc5ccccc5cc4-c4ccccc4)cc3)c3ccc(N4c5ccccc5Sc5ccccc54)cc3)cc2)cc1 ; c1ccc(-c2ccc(N(c3ccc(-c4cc5ccccc5cc4-c4ccccc4)cc3)c3ccc(N4c5ccccc5-c5cccc6cccc4c56)cc3)cc2)cc1 ; c1ccc(-c2ccc(N(c3ccc(-c4ccc5c(c4)C4(c6ccccc6O5)c5ccccc5-c5ccccc54)cc3)c3ccc(-c4cc5ccccc5cc4-c4ccccc4)cc3)cc2)cc1]

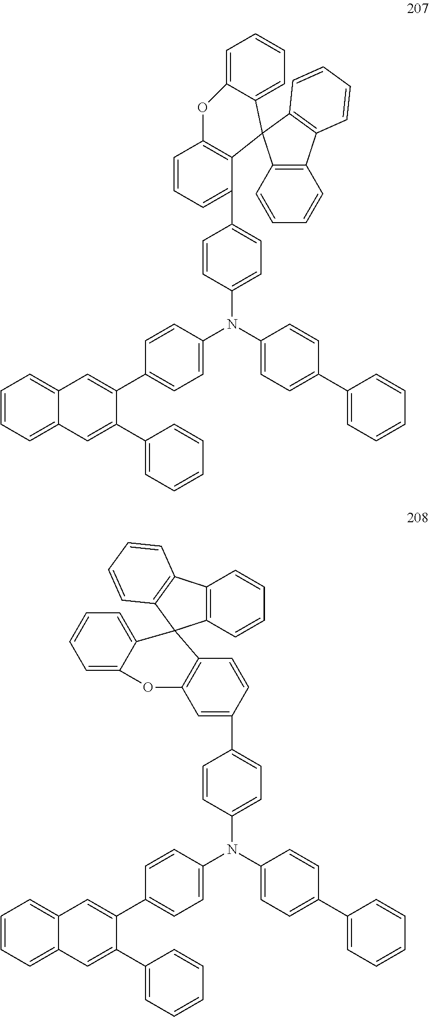 [SMILES: c1ccc(-c2ccc(N(c3ccc(-c4cc5ccccc5cc4-c4ccccc4)cc3)c3ccc(-c4cccc5c4C4(c6ccccc6O5)c5ccccc5-c5ccccc54)cc3)cc2)cc1 ; c1ccc(-c2ccc(N(c3ccc(-c4ccc5c(c4)Oc4ccccc4C54c5ccccc5-c5ccccc54)cc3)c3ccc(-c4cc5ccccc5cc4-c4ccccc4)cc3)cc2)cc1]

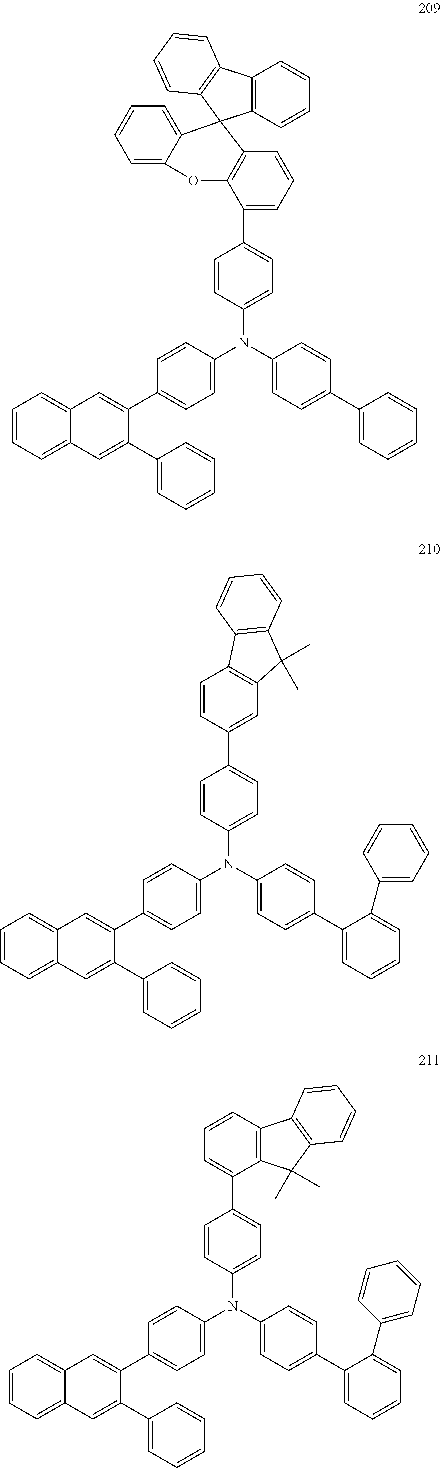 [SMILES: c1ccc(-c2ccc(N(c3ccc(-c4cc5ccccc5cc4-c4ccccc4)cc3)c3ccc(-c4cccc5c4Oc4ccccc4C54c5ccccc5-c5ccccc54)cc3)cc2)cc1 ; CC1(C)c2ccccc2-c2ccc(-c3ccc(N(c4ccc(-c5ccccc5-c5ccccc5)cc4)c4ccc(-c5cc6ccccc6cc5-c5ccccc5)cc4)cc3)cc21 ; CC1(C)c2ccccc2-c2cccc(-c3ccc(N(c4ccc(-c5ccccc5-c5ccccc5)cc4)c4ccc(-c5cc6ccccc6cc5-c5ccccc5)cc4)cc3)c21]

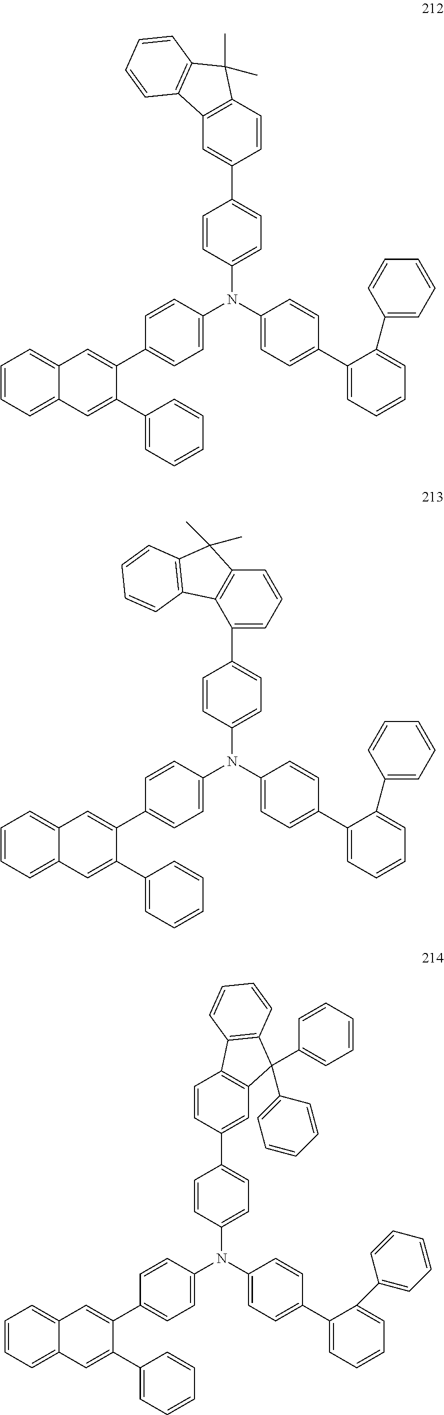 [SMILES: CC1(C)c2ccccc2-c2cc(-c3ccc(N(c4ccc(-c5ccccc5-c5ccccc5)cc4)c4ccc(-c5cc6ccccc6cc5-c5ccccc5)cc4)cc3)ccc21 ; CC1(C)c2ccccc2-c2c(-c3ccc(N(c4ccc(-c5ccccc5-c5ccccc5)cc4)c4ccc(-c5cc6ccccc6cc5-c5ccccc5)cc4)cc3)cccc21 ; c1ccc(-c2ccccc2-c2ccc(N(c3ccc(-c4ccc5c(c4)C(c4ccccc4)(c4ccccc4)c4ccccc4-5)cc3)c3ccc(-c4cc5ccccc5cc4-c4ccccc4)cc3)cc2)cc1]

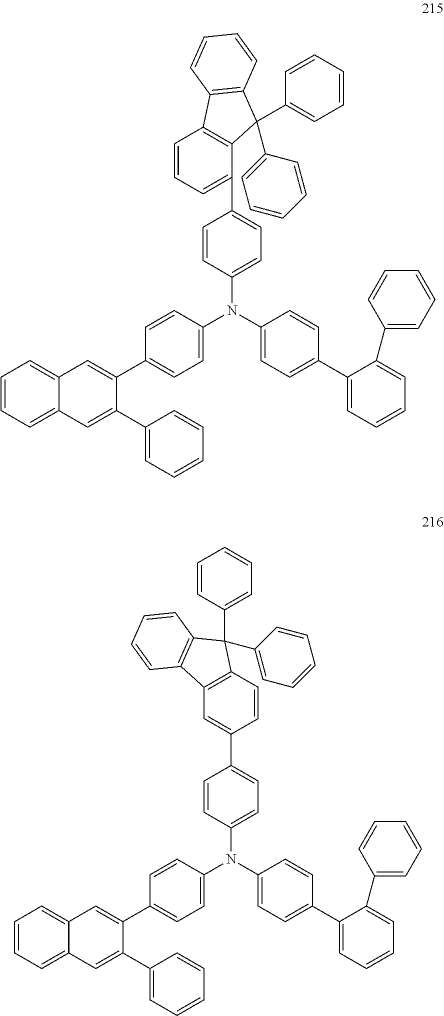 [SMILES: c1ccc(-c2ccccc2-c2ccc(N(c3ccc(-c4cc5ccccc5cc4-c4ccccc4)cc3)c3ccc(-c4cccc5c4C(c4ccccc4)(c4ccccc4)c4ccccc4-5)cc3)cc2)cc1 ; c1ccc(-c2ccccc2-c2ccc(N(c3ccc(-c4ccc5c(c4)-c4ccccc4C5(c4ccccc4)c4ccccc4)cc3)c3ccc(-c4cc5ccccc5cc4-c4ccccc4)cc3)cc2)cc1]

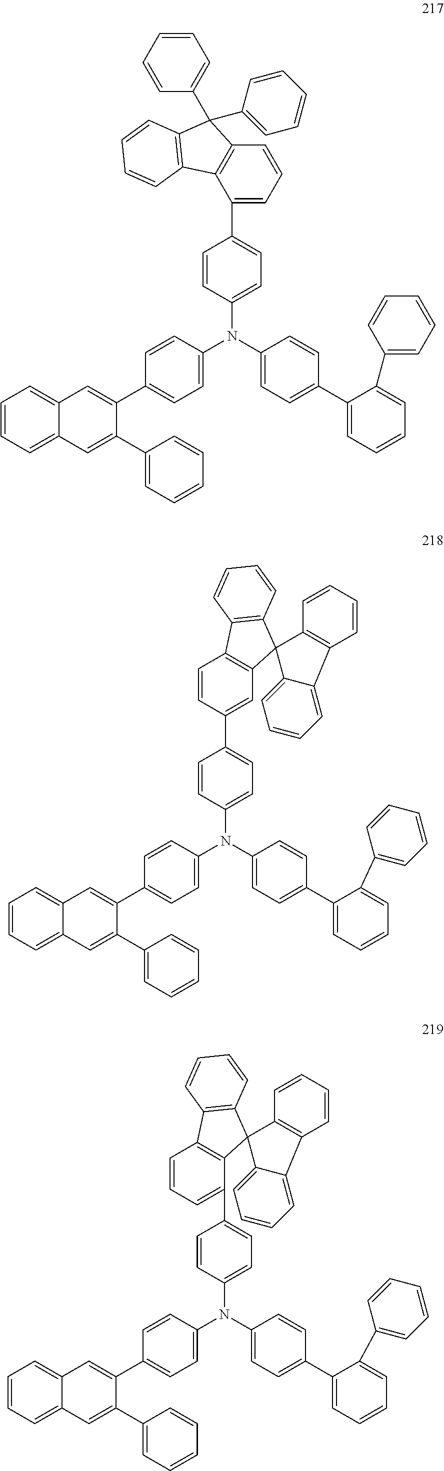 [SMILES: c1ccc(-c2ccccc2-c2ccc(N(c3ccc(-c4cc5ccccc5cc4-c4ccccc4)cc3)c3ccc(-c4cccc5c4-c4ccccc4C5(c4ccccc4)c4ccccc4)cc3)cc2)cc1 ; c1ccc(-c2ccccc2-c2ccc(N(c3ccc(-c4ccc5c(c4)C4(c6ccccc6-c6ccccc64)c4ccccc4-5)cc3)c3ccc(-c4cc5ccccc5cc4-c4ccccc4)cc3)cc2)cc1 ; c1ccc(-c2ccccc2-c2ccc(N(c3ccc(-c4cc5ccccc5cc4-c4ccccc4)cc3)c3ccc(-c4cccc5c4C4(c6ccccc6-c6ccccc64)c4ccccc4-5)cc3)cc2)cc1]

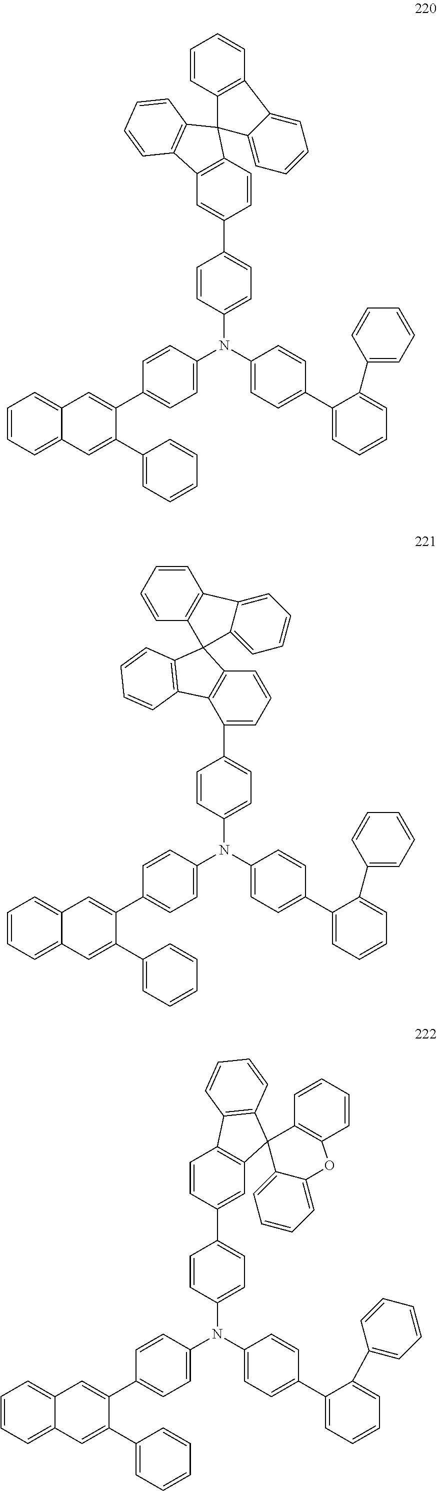 [SMILES: c1ccc(-c2ccccc2-c2ccc(N(c3ccc(-c4ccc5c(c4)-c4ccccc4C54c5ccccc5-c5ccccc54)cc3)c3ccc(-c4cc5ccccc5cc4-c4ccccc4)cc3)cc2)cc1 ; c1ccc(-c2ccccc2-c2ccc(N(c3ccc(-c4cc5ccccc5cc4-c4ccccc4)cc3)c3ccc(-c4cccc5c4-c4ccccc4C54c5ccccc5-c5ccccc54)cc3)cc2)cc1 ; c1ccc(-c2ccccc2-c2ccc(N(c3ccc(-c4ccc5c(c4)C4(c6ccccc6Oc6ccccc64)c4ccccc4-5)cc3)c3ccc(-c4cc5ccccc5cc4-c4ccccc4)cc3)cc2)cc1]

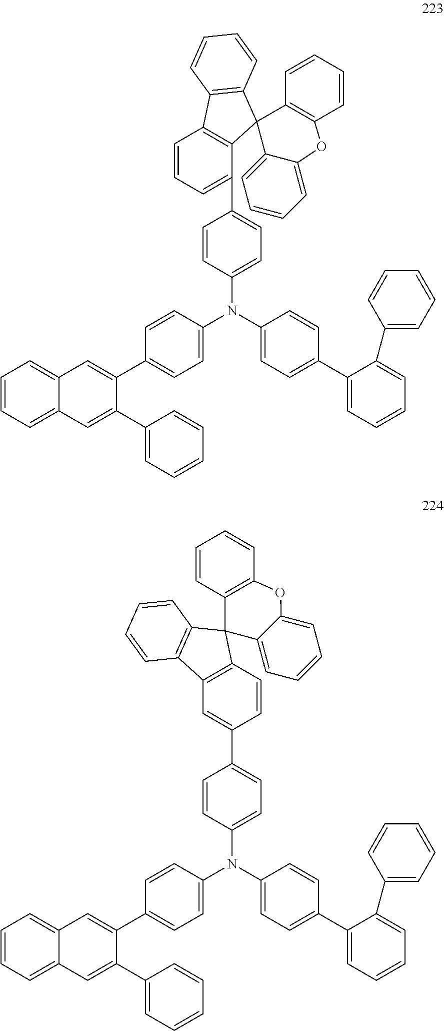 [SMILES: c1ccc(-c2ccccc2-c2ccc(N(c3ccc(-c4cc5ccccc5cc4-c4ccccc4)cc3)c3ccc(-c4cccc5c4C4(c6ccccc6Oc6ccccc64)c4ccccc4-5)cc3)cc2)cc1 ; c1ccc(-c2ccccc2-c2ccc(N(c3ccc(-c4ccc5c(c4)-c4ccccc4C54c5ccccc5Oc5ccccc54)cc3)c3ccc(-c4cc5ccccc5cc4-c4ccccc4)cc3)cc2)cc1]

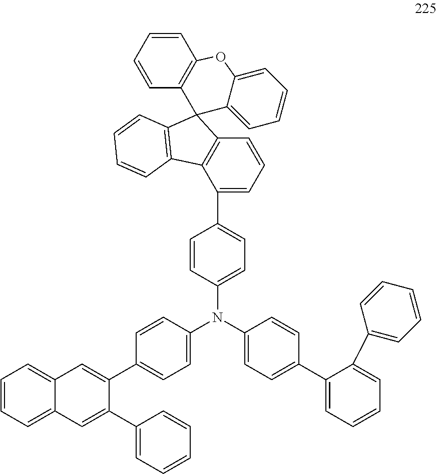 [SMILES: c1ccc(-c2ccccc2-c2ccc(N(c3ccc(-c4cc5ccccc5cc4-c4ccccc4)cc3)c3ccc(-c4cccc5c4-c4ccccc4C54c5ccccc5Oc5ccccc54)cc3)cc2)cc1]

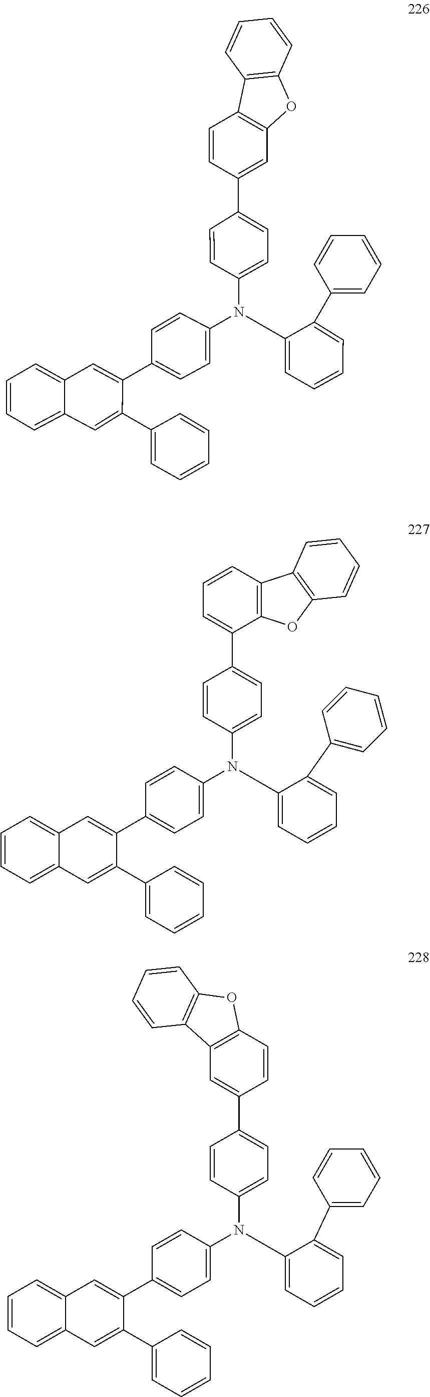

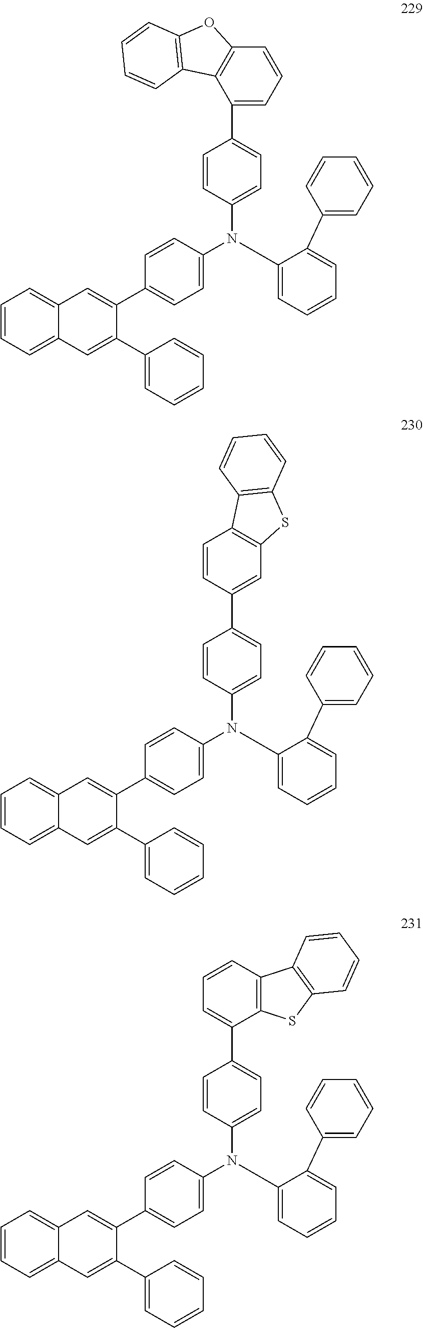 [SMILES: c1ccc(-c2cc3ccccc3cc2-c2ccc(N(c3ccc(-c4cccc5oc6ccccc6c45)cc3)c3ccccc3-c3ccccc3)cc2)cc1 ; c1ccc(-c2cc3ccccc3cc2-c2ccc(N(c3ccc(-c4ccc5c(c4)sc4ccccc45)cc3)c3ccccc3-c3ccccc3)cc2)cc1 ; c1ccc(-c2cc3ccccc3cc2-c2ccc(N(c3ccc(-c4cccc5c4sc4ccccc45)cc3)c3ccccc3-c3ccccc3)cc2)cc1]

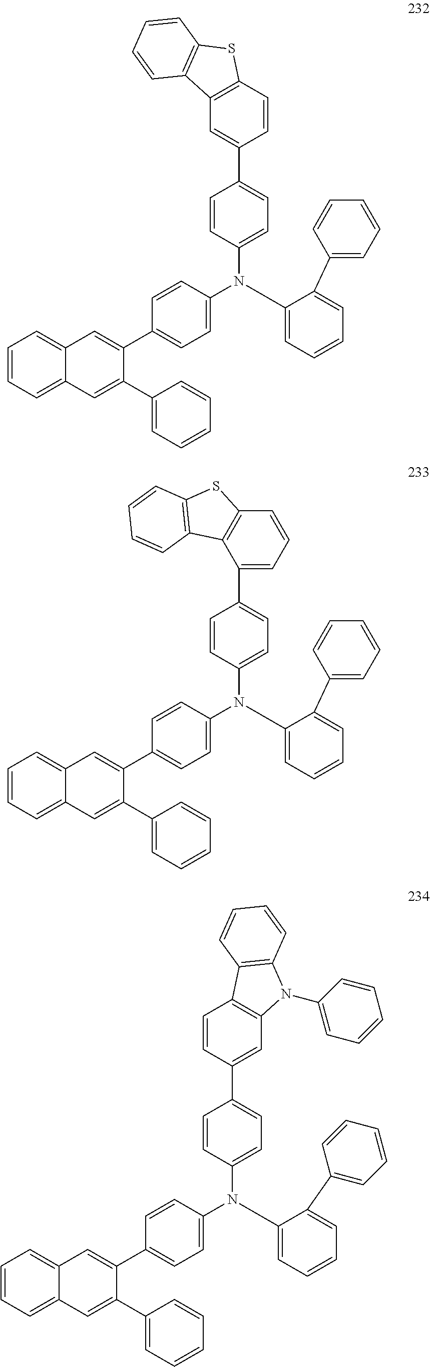

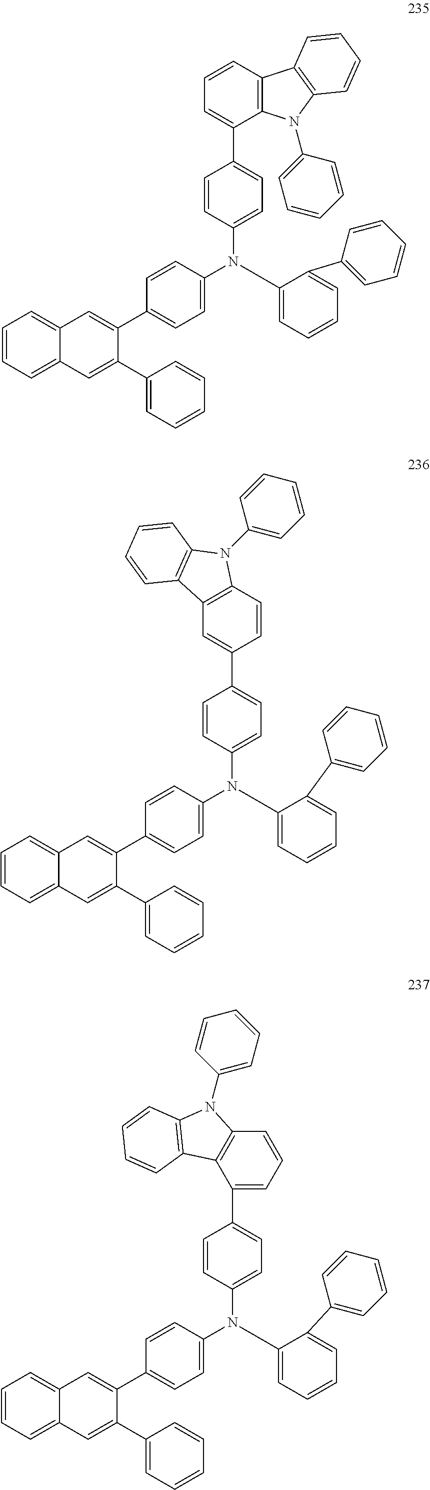 [SMILES: c1ccc(-c2cc3ccccc3cc2-c2ccc(N(c3ccc(-c4cccc5c6ccccc6n(-c6ccccc6)c45)cc3)c3ccccc3-c3ccccc3)cc2)cc1 ; c1ccc(-c2cc3ccccc3cc2-c2ccc(N(c3ccc(-c4ccc5c(c4)c4ccccc4n5-c4ccccc4)cc3)c3ccccc3-c3ccccc3)cc2)cc1 ; c1ccc(-c2cc3ccccc3cc2-c2ccc(N(c3ccc(-c4cccc5c4c4ccccc4n5-c4ccccc4)cc3)c3ccccc3-c3ccccc3)cc2)cc1]

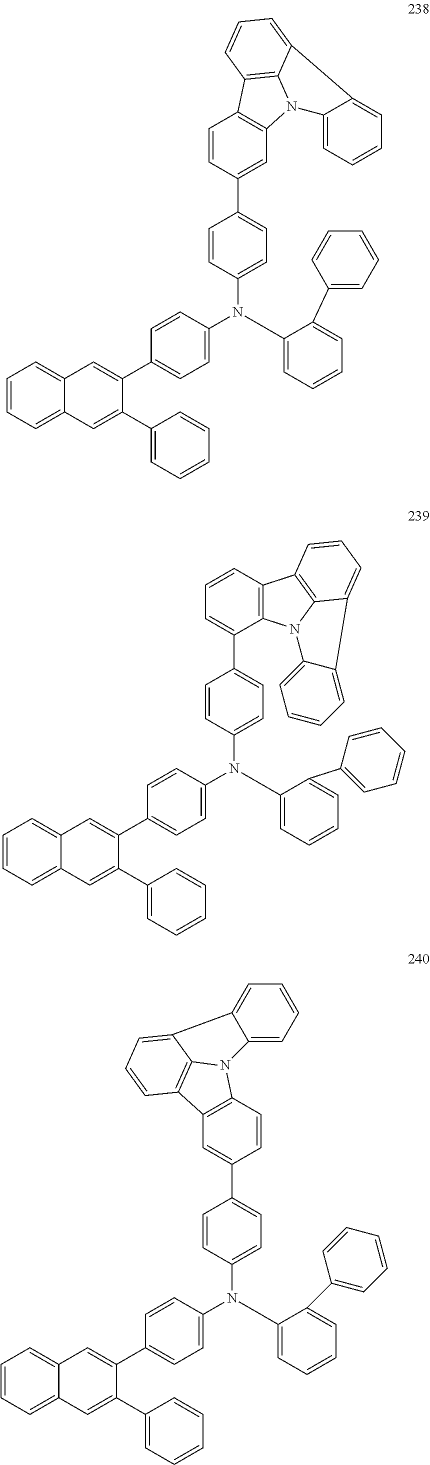 [SMILES: c1ccc(-c2cc3ccccc3cc2-c2ccc(N(c3ccc(-c4ccc5c6cccc7c8ccccc8n(c5c4)c76)cc3)c3ccccc3-c3ccccc3)cc2)cc1 ; c1ccc(-c2cc3ccccc3cc2-c2ccc(N(c3ccc(-c4cccc5c6cccc7c8ccccc8n(c45)c76)cc3)c3ccccc3-c3ccccc3)cc2)cc1 ; c1ccc(-c2cc3ccccc3cc2-c2ccc(N(c3ccc(-c4ccc5c(c4)c4cccc6c7ccccc7n5c64)cc3)c3ccccc3-c3ccccc3)cc2)cc1]

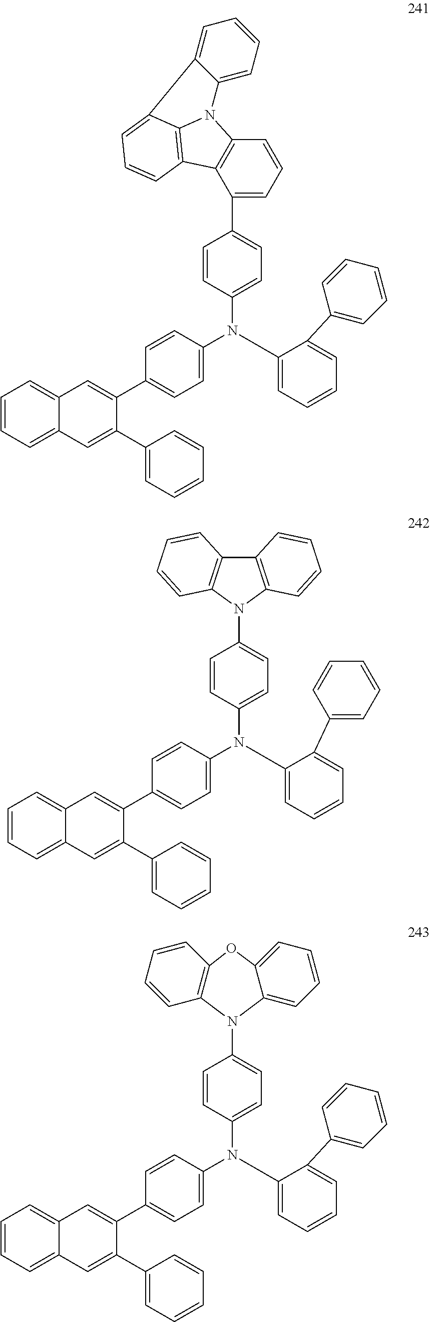

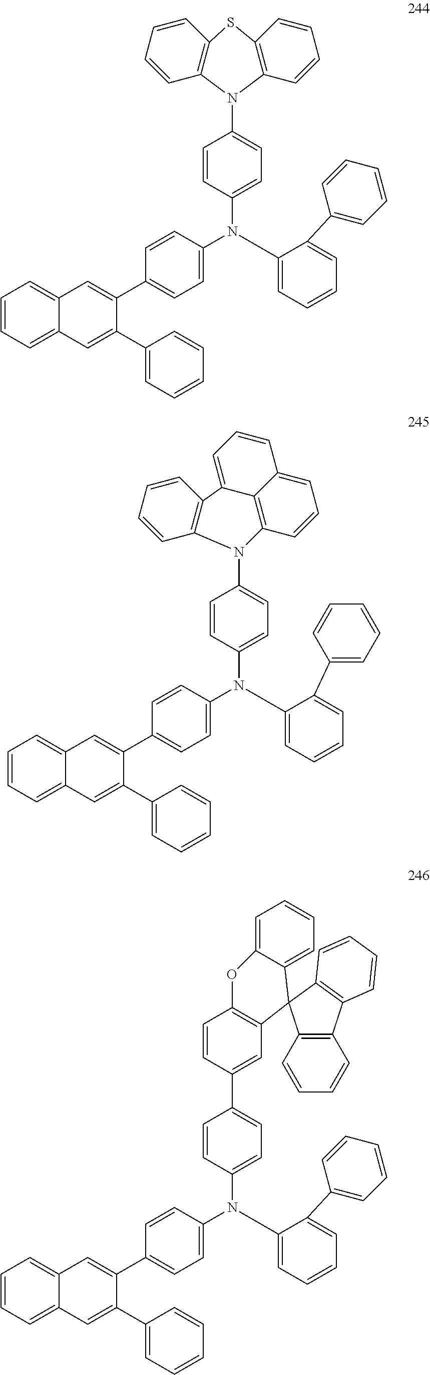 [SMILES: c1ccc(-c2cc3ccccc3cc2-c2ccc(N(c3ccc(N4c5ccccc5Sc5ccccc54)cc3)c3ccccc3-c3ccccc3)cc2)cc1 ; c1ccc(-c2cc3ccccc3cc2-c2ccc(N(c3ccc(N4c5ccccc5-c5cccc6cccc4c56)cc3)c3ccccc3-c3ccccc3)cc2)cc1 ; c1ccc(-c2cc3ccccc3cc2-c2ccc(N(c3ccc(-c4ccc5c(c4)C4(c6ccccc6O5)c5ccccc5-c5ccccc54)cc3)c3ccccc3-c3ccccc3)cc2)cc1]

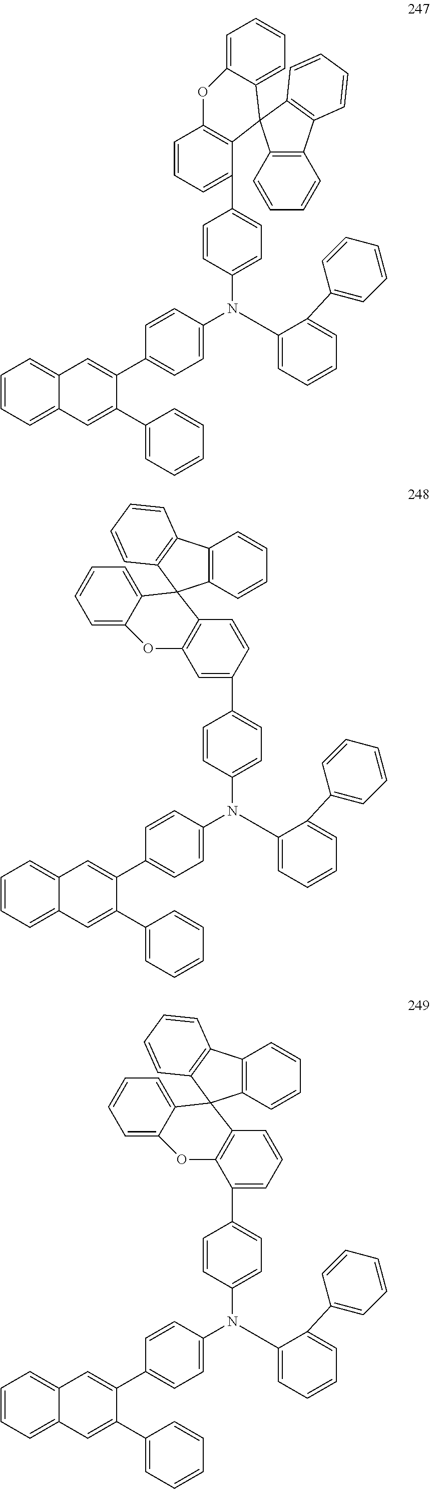

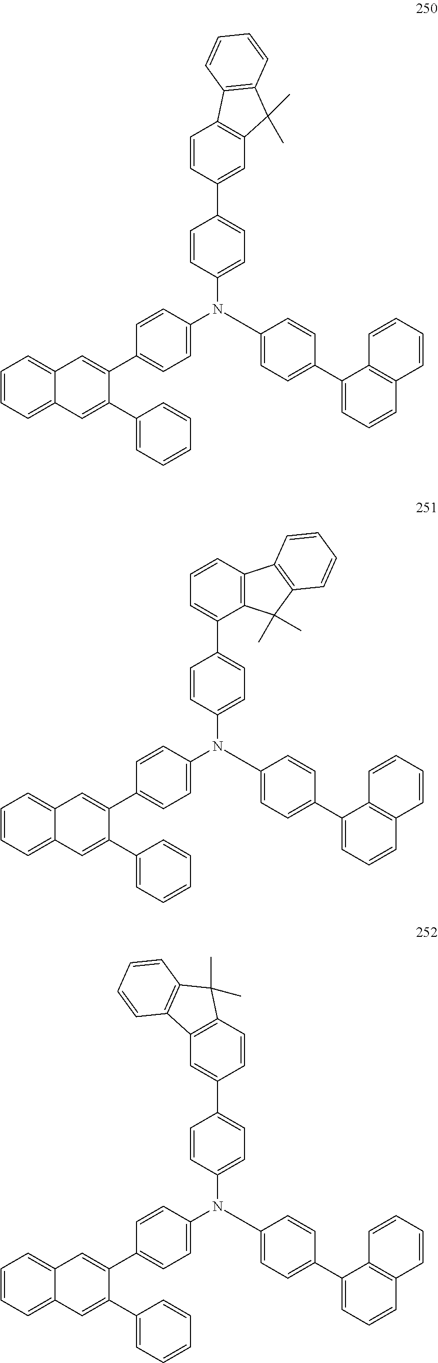 [SMILES: CC1(C)c2ccccc2-c2ccc(-c3ccc(N(c4ccc(-c5cc6ccccc6cc5-c5ccccc5)cc4)c4ccc(-c5cccc6ccccc56)cc4)cc3)cc21 ; CC1(C)c2ccccc2-c2cccc(-c3ccc(N(c4ccc(-c5cc6ccccc6cc5-c5ccccc5)cc4)c4ccc(-c5cccc6ccccc56)cc4)cc3)c21 ; CC1(C)c2ccccc2-c2cc(-c3ccc(N(c4ccc(-c5cc6ccccc6cc5-c5ccccc5)cc4)c4ccc(-c5cccc6ccccc56)cc4)cc3)ccc21]

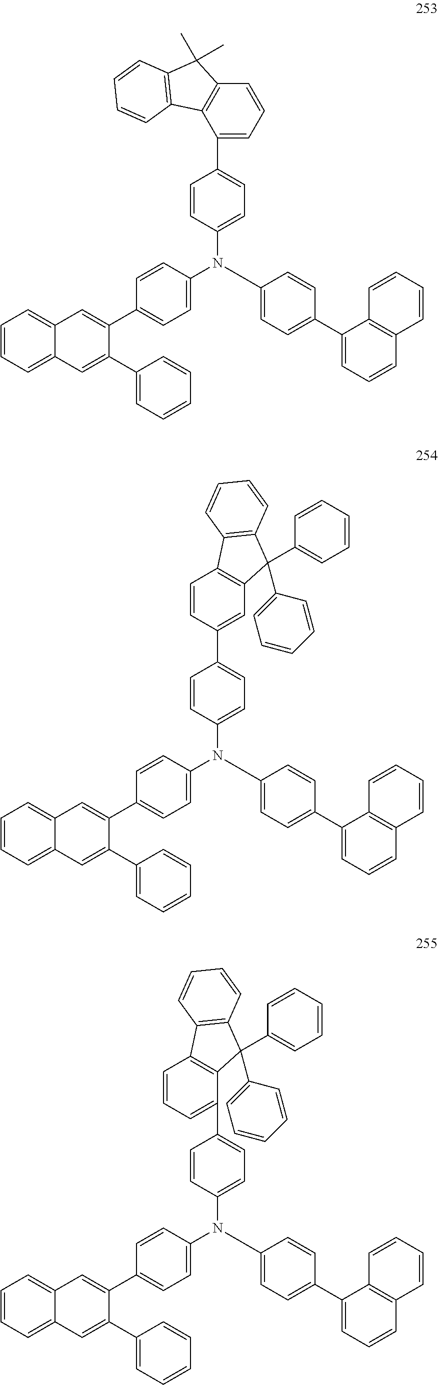 [SMILES: CC1(C)c2ccccc2-c2c(-c3ccc(N(c4ccc(-c5cc6ccccc6cc5-c5ccccc5)cc4)c4ccc(-c5cccc6ccccc56)cc4)cc3)cccc21 ; c1ccc(-c2cc3ccccc3cc2-c2ccc(N(c3ccc(-c4ccc5c(c4)C(c4ccccc4)(c4ccccc4)c4ccccc4-5)cc3)c3ccc(-c4cccc5ccccc45)cc3)cc2)cc1 ; c1ccc(-c2cc3ccccc3cc2-c2ccc(N(c3ccc(-c4cccc5c4C(c4ccccc4)(c4ccccc4)c4ccccc4-5)cc3)c3ccc(-c4cccc5ccccc45)cc3)cc2)cc1]

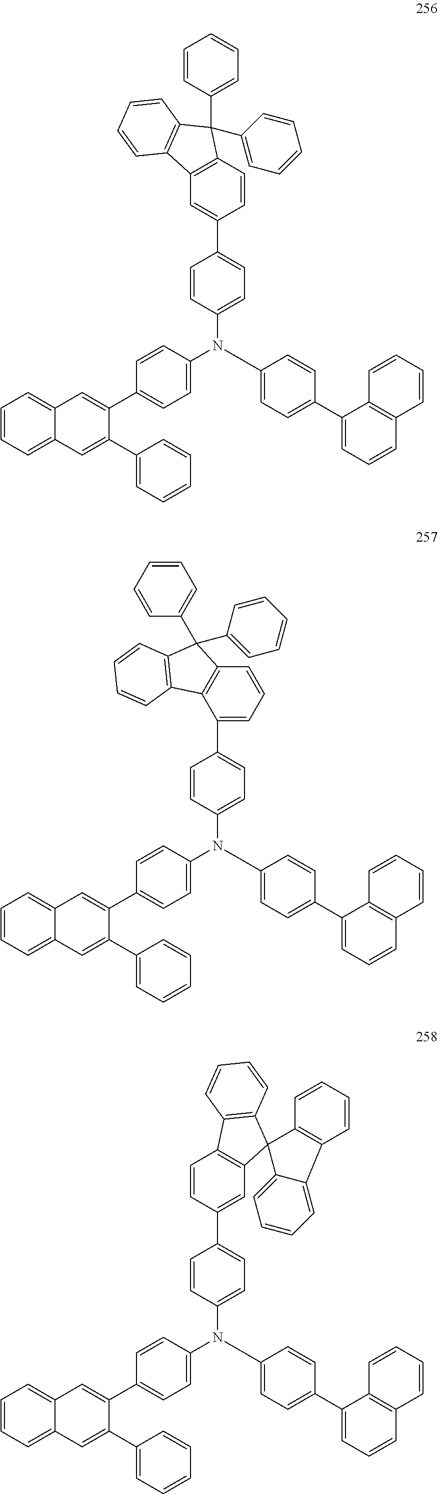 [SMILES: c1ccc(-c2cc3ccccc3cc2-c2ccc(N(c3ccc(-c4ccc5c(c4)-c4ccccc4C5(c4ccccc4)c4ccccc4)cc3)c3ccc(-c4cccc5ccccc45)cc3)cc2)cc1 ; c1ccc(-c2cc3ccccc3cc2-c2ccc(N(c3ccc(-c4cccc5c4-c4ccccc4C5(c4ccccc4)c4ccccc4)cc3)c3ccc(-c4cccc5ccccc45)cc3)cc2)cc1 ; c1ccc(-c2cc3ccccc3cc2-c2ccc(N(c3ccc(-c4ccc5c(c4)C4(c6ccccc6-c6ccccc64)c4ccccc4-5)cc3)c3ccc(-c4cccc5ccccc45)cc3)cc2)cc1]

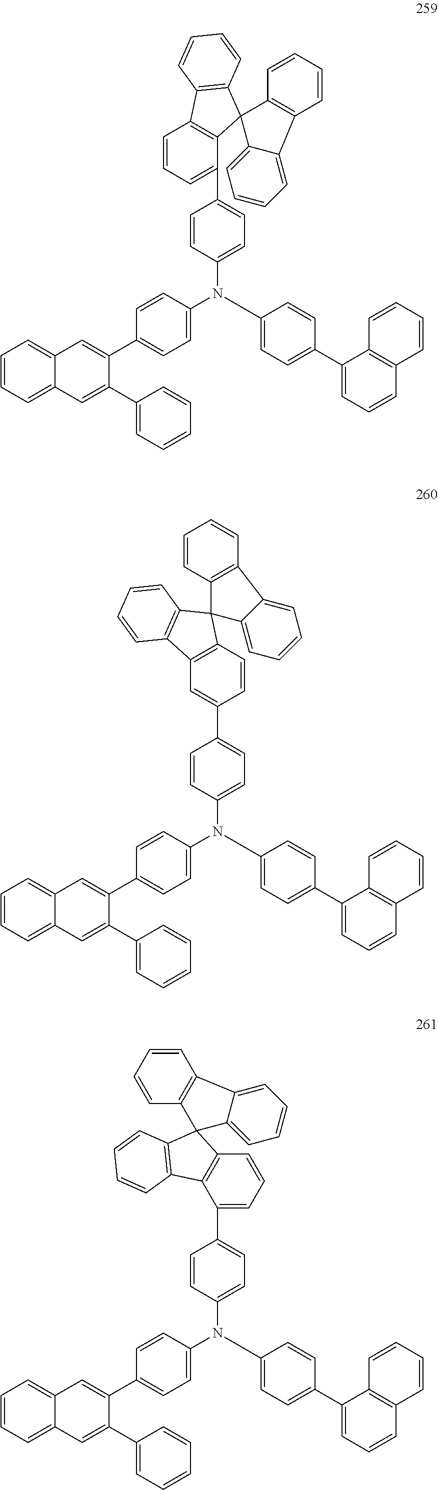 [SMILES: c1ccc(-c2cc3ccccc3cc2-c2ccc(N(c3ccc(-c4cccc5c4C4(c6ccccc6-c6ccccc64)c4ccccc4-5)cc3)c3ccc(-c4cccc5ccccc45)cc3)cc2)cc1 ; c1ccc(-c2cc3ccccc3cc2-c2ccc(N(c3ccc(-c4ccc5c(c4)-c4ccccc4C54c5ccccc5-c5ccccc54)cc3)c3ccc(-c4cccc5ccccc45)cc3)cc2)cc1 ; c1ccc(-c2cc3ccccc3cc2-c2ccc(N(c3ccc(-c4cccc5c4-c4ccccc4C54c5ccccc5-c5ccccc54)cc3)c3ccc(-c4cccc5ccccc45)cc3)cc2)cc1]

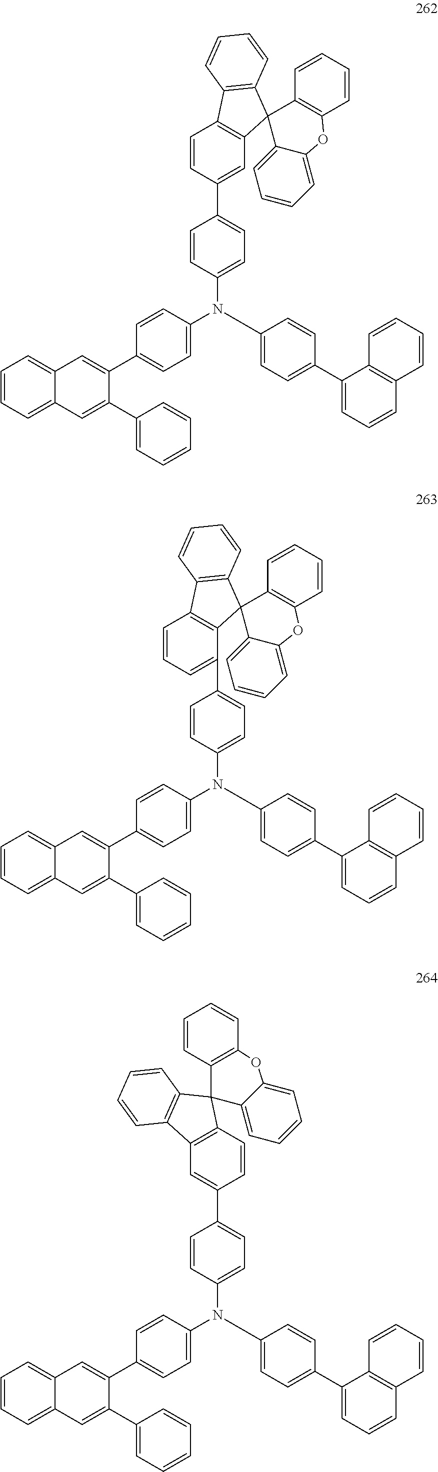 [SMILES: c1ccc(-c2cc3ccccc3cc2-c2ccc(N(c3ccc(-c4ccc5c(c4)C4(c6ccccc6Oc6ccccc64)c4ccccc4-5)cc3)c3ccc(-c4cccc5ccccc45)cc3)cc2)cc1 ; c1ccc(-c2cc3ccccc3cc2-c2ccc(N(c3ccc(-c4cccc5c4C4(c6ccccc6Oc6ccccc64)c4ccccc4-5)cc3)c3ccc(-c4cccc5ccccc45)cc3)cc2)cc1 ; c1ccc(-c2cc3ccccc3cc2-c2ccc(N(c3ccc(-c4ccc5c(c4)-c4ccccc4C54c5ccccc5Oc5ccccc54)cc3)c3ccc(-c4cccc5ccccc45)cc3)cc2)cc1]

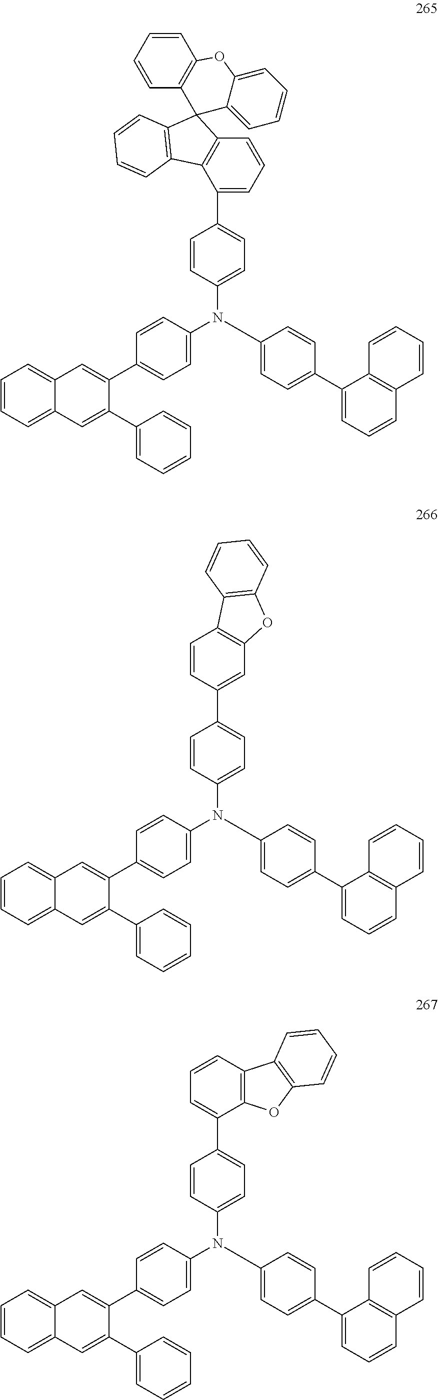 [SMILES: c1ccc(-c2cc3ccccc3cc2-c2ccc(N(c3ccc(-c4cccc5c4-c4ccccc4C54c5ccccc5Oc5ccccc54)cc3)c3ccc(-c4cccc5ccccc45)cc3)cc2)cc1 ; c1ccc(-c2cc3ccccc3cc2-c2ccc(N(c3ccc(-c4ccc5c(c4)oc4ccccc45)cc3)c3ccc(-c4cccc5ccccc45)cc3)cc2)cc1 ; c1ccc(-c2cc3ccccc3cc2-c2ccc(N(c3ccc(-c4cccc5ccccc45)cc3)c3ccc(-c4cccc5c4oc4ccccc45)cc3)cc2)cc1]

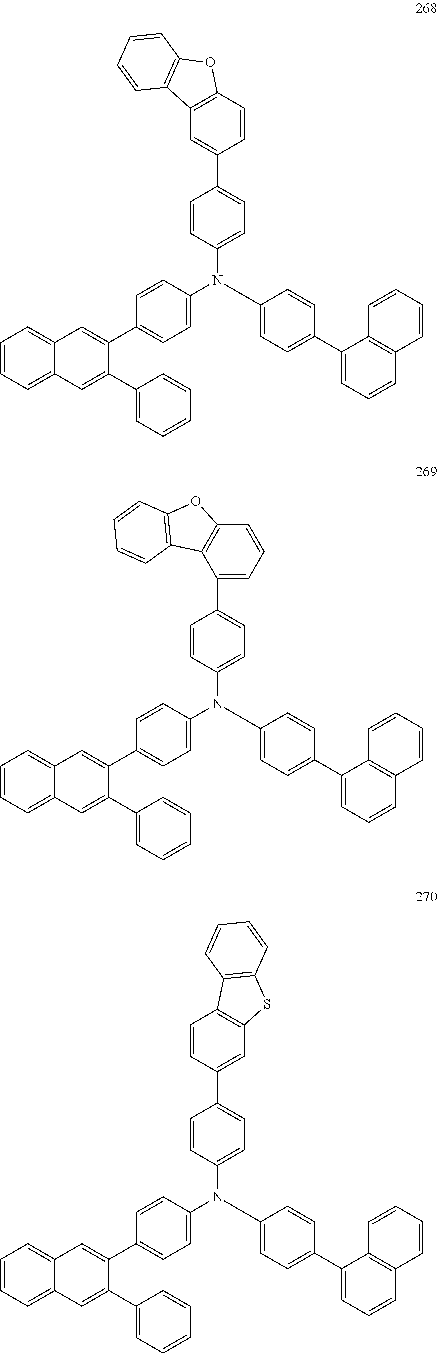 [SMILES: c1ccc(-c2cc3ccccc3cc2-c2ccc(N(c3ccc(-c4ccc5oc6ccccc6c5c4)cc3)c3ccc(-c4cccc5ccccc45)cc3)cc2)cc1 ; c1ccc(-c2cc3ccccc3cc2-c2ccc(N(c3ccc(-c4cccc5ccccc45)cc3)c3ccc(-c4cccc5oc6ccccc6c45)cc3)cc2)cc1 ; c1ccc(-c2cc3ccccc3cc2-c2ccc(N(c3ccc(-c4ccc5c(c4)sc4ccccc45)cc3)c3ccc(-c4cccc5ccccc45)cc3)cc2)cc1]

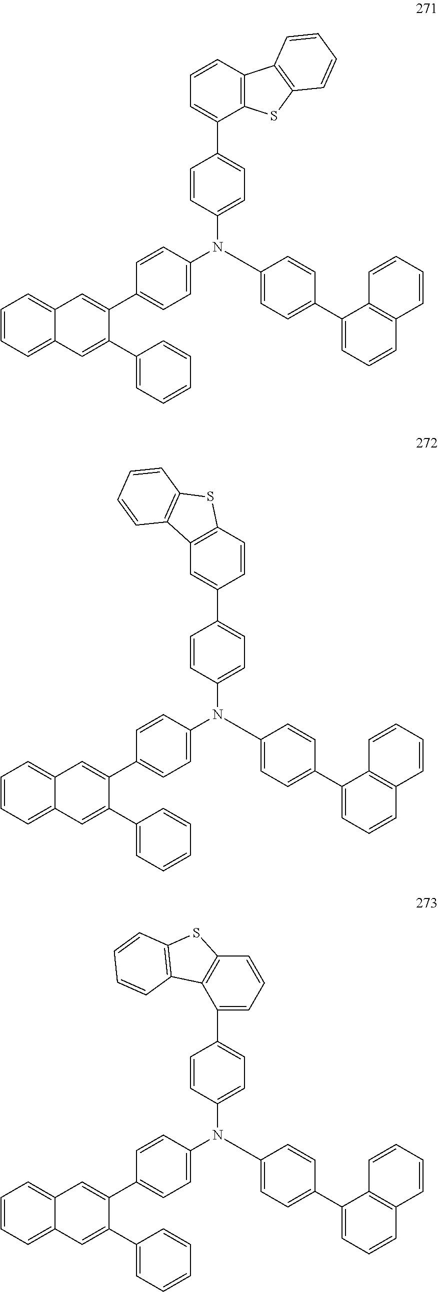 [SMILES: c1ccc(-c2cc3ccccc3cc2-c2ccc(N(c3ccc(-c4cccc5ccccc45)cc3)c3ccc(-c4cccc5c4sc4ccccc45)cc3)cc2)cc1 ; c1ccc(-c2cc3ccccc3cc2-c2ccc(N(c3ccc(-c4ccc5sc6ccccc6c5c4)cc3)c3ccc(-c4cccc5ccccc45)cc3)cc2)cc1 ; c1ccc(-c2cc3ccccc3cc2-c2ccc(N(c3ccc(-c4cccc5ccccc45)cc3)c3ccc(-c4cccc5sc6ccccc6c45)cc3)cc2)cc1]

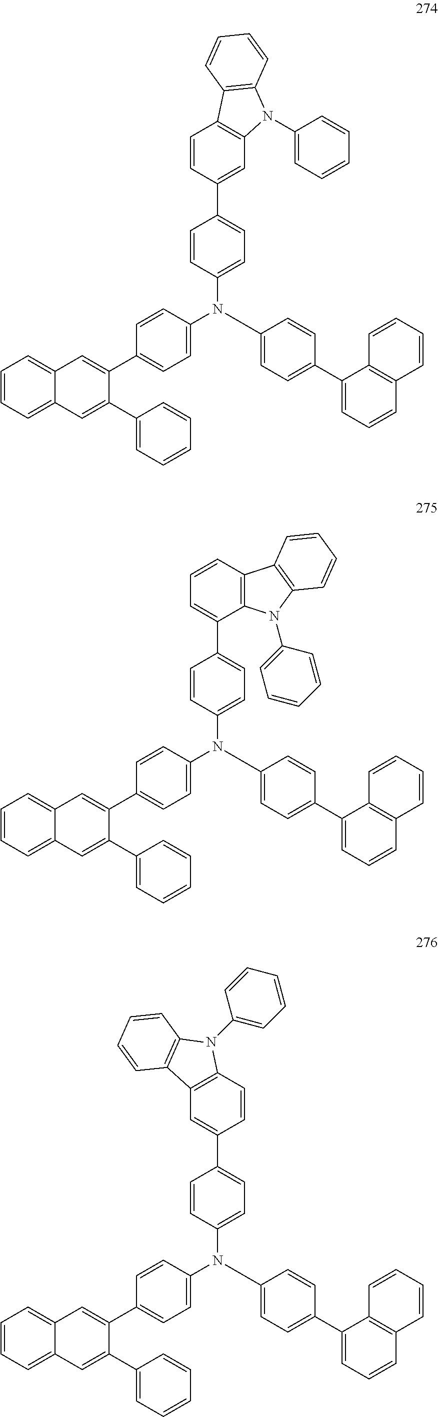 [SMILES: c1ccc(-c2cc3ccccc3cc2-c2ccc(N(c3ccc(-c4ccc5c6ccccc6n(-c6ccccc6)c5c4)cc3)c3ccc(-c4cccc5ccccc45)cc3)cc2)cc1 ; c1ccc(-c2cc3ccccc3cc2-c2ccc(N(c3ccc(-c4cccc5ccccc45)cc3)c3ccc(-c4cccc5c6ccccc6n(-c6ccccc6)c45)cc3)cc2)cc1 ; c1ccc(-c2cc3ccccc3cc2-c2ccc(N(c3ccc(-c4ccc5c(c4)c4ccccc4n5-c4ccccc4)cc3)c3ccc(-c4cccc5ccccc45)cc3)cc2)cc1]

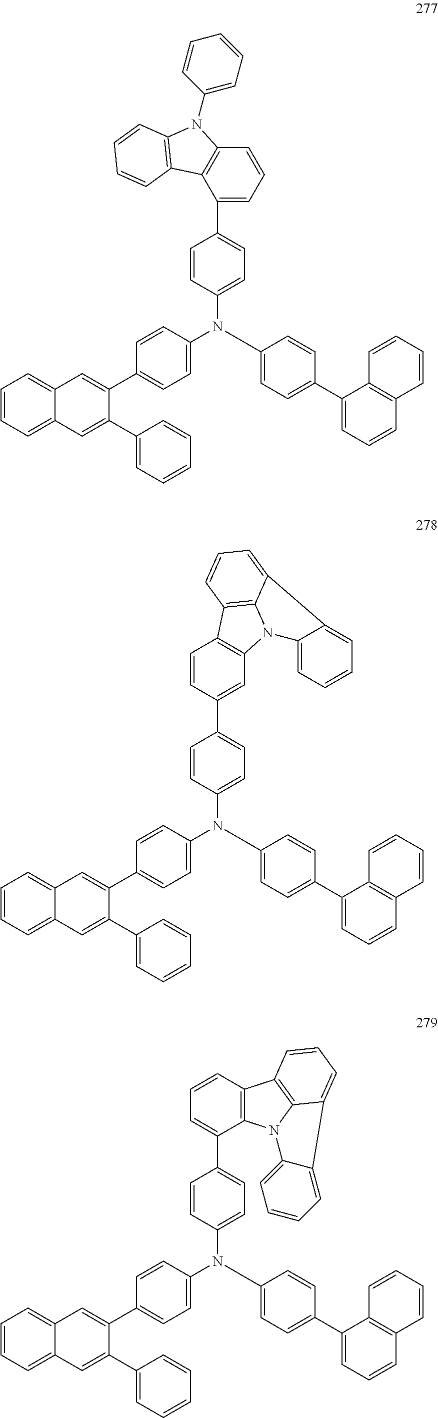 [SMILES: c1ccc(-c2cc3ccccc3cc2-c2ccc(N(c3ccc(-c4cccc5ccccc45)cc3)c3ccc(-c4cccc5c4c4ccccc4n5-c4ccccc4)cc3)cc2)cc1 ; c1ccc(-c2cc3ccccc3cc2-c2ccc(N(c3ccc(-c4ccc5c6cccc7c8ccccc8n(c5c4)c76)cc3)c3ccc(-c4cccc5ccccc45)cc3)cc2)cc1 ; c1ccc(-c2cc3ccccc3cc2-c2ccc(N(c3ccc(-c4cccc5ccccc45)cc3)c3ccc(-c4cccc5c6cccc7c8ccccc8n(c45)c76)cc3)cc2)cc1]

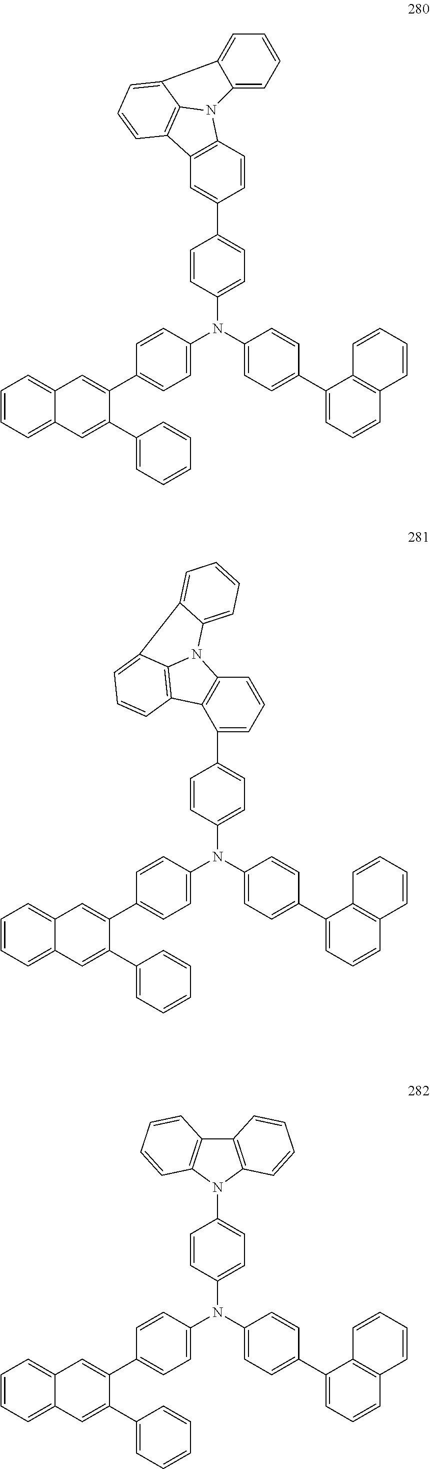 [SMILES: c1ccc(-c2cc3ccccc3cc2-c2ccc(N(c3ccc(-c4ccc5c(c4)c4cccc6c7ccccc7n5c64)cc3)c3ccc(-c4cccc5ccccc45)cc3)cc2)cc1 ; c1ccc(-c2cc3ccccc3cc2-c2ccc(N(c3ccc(-c4cccc5ccccc45)cc3)c3ccc(-c4cccc5c4c4cccc6c7ccccc7n5c64)cc3)cc2)cc1 ; c1ccc(-c2cc3ccccc3cc2-c2ccc(N(c3ccc(-c4cccc5ccccc45)cc3)c3ccc(-n4c5ccccc5c5ccccc54)cc3)cc2)cc1]

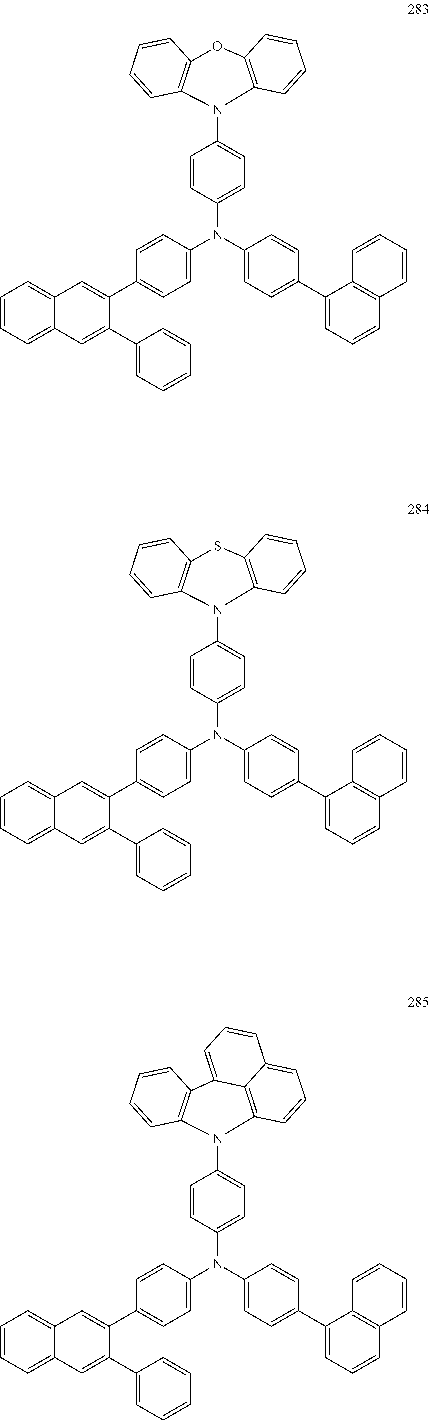 [SMILES: c1ccc(-c2cc3ccccc3cc2-c2ccc(N(c3ccc(-c4cccc5ccccc45)cc3)c3ccc(N4c5ccccc5Oc5ccccc54)cc3)cc2)cc1 ; c1ccc(-c2cc3ccccc3cc2-c2ccc(N(c3ccc(-c4cccc5ccccc45)cc3)c3ccc(N4c5ccccc5Sc5ccccc54)cc3)cc2)cc1 ; c1ccc(-c2cc3ccccc3cc2-c2ccc(N(c3ccc(-c4cccc5ccccc45)cc3)c3ccc(N4c5ccccc5-c5cccc6cccc4c56)cc3)cc2)cc1]

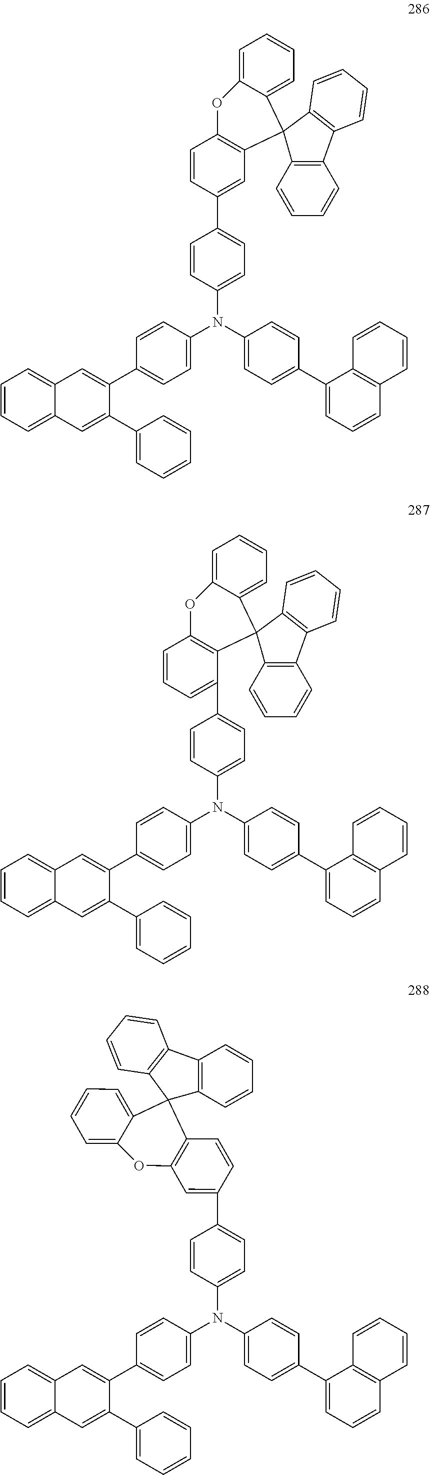 [SMILES: c1ccc(-c2cc3ccccc3cc2-c2ccc(N(c3ccc(-c4ccc5c(c4)C4(c6ccccc6O5)c5ccccc5-c5ccccc54)cc3)c3ccc(-c4cccc5ccccc45)cc3)cc2)cc1 ; c1ccc(-c2cc3ccccc3cc2-c2ccc(N(c3ccc(-c4cccc5c4C4(c6ccccc6O5)c5ccccc5-c5ccccc54)cc3)c3ccc(-c4cccc5ccccc45)cc3)cc2)cc1 ; c1ccc(-c2cc3ccccc3cc2-c2ccc(N(c3ccc(-c4ccc5c(c4)Oc4ccccc4C54c5ccccc5-c5ccccc54)cc3)c3ccc(-c4cccc5ccccc45)cc3)cc2)cc1]

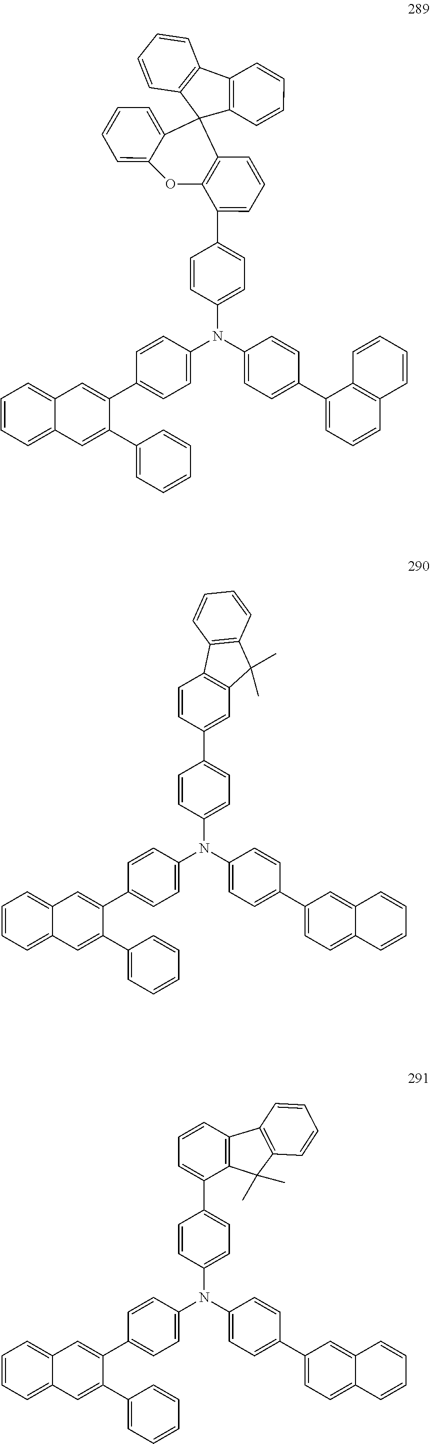 [SMILES: c1ccc(-c2cc3ccccc3cc2-c2ccc(N(c3ccc(-c4cccc5c4Oc4ccccc4C54c5ccccc5-c5ccccc54)cc3)c3ccc(-c4cccc5ccccc45)cc3)cc2)cc1 ; CC1(C)c2ccccc2-c2ccc(-c3ccc(N(c4ccc(-c5ccc6ccccc6c5)cc4)c4ccc(-c5cc6ccccc6cc5-c5ccccc5)cc4)cc3)cc21 ; CC1(C)c2ccccc2-c2cccc(-c3ccc(N(c4ccc(-c5ccc6ccccc6c5)cc4)c4ccc(-c5cc6ccccc6cc5-c5ccccc5)cc4)cc3)c21]

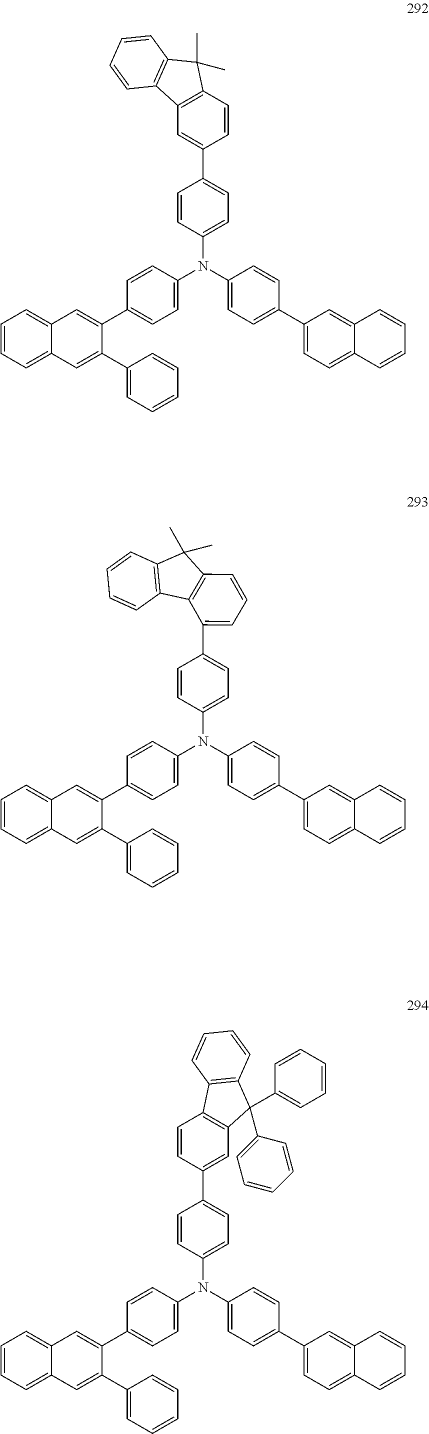 [SMILES: CC1(C)c2ccccc2-c2cc(-c3ccc(N(c4ccc(-c5ccc6ccccc6c5)cc4)c4ccc(-c5cc6ccccc6cc5-c5ccccc5)cc4)cc3)ccc21 ; CC1(C)c2ccccc2-c2c(-c3ccc(N(c4ccc(-c5ccc6ccccc6c5)cc4)c4ccc(-c5cc6ccccc6cc5-c5ccccc5)cc4)cc3)cccc21 ; c1ccc(-c2cc3ccccc3cc2-c2ccc(N(c3ccc(-c4ccc5c(c4)C(c4ccccc4)(c4ccccc4)c4ccccc4-5)cc3)c3ccc(-c4ccc5ccccc5c4)cc3)cc2)cc1]

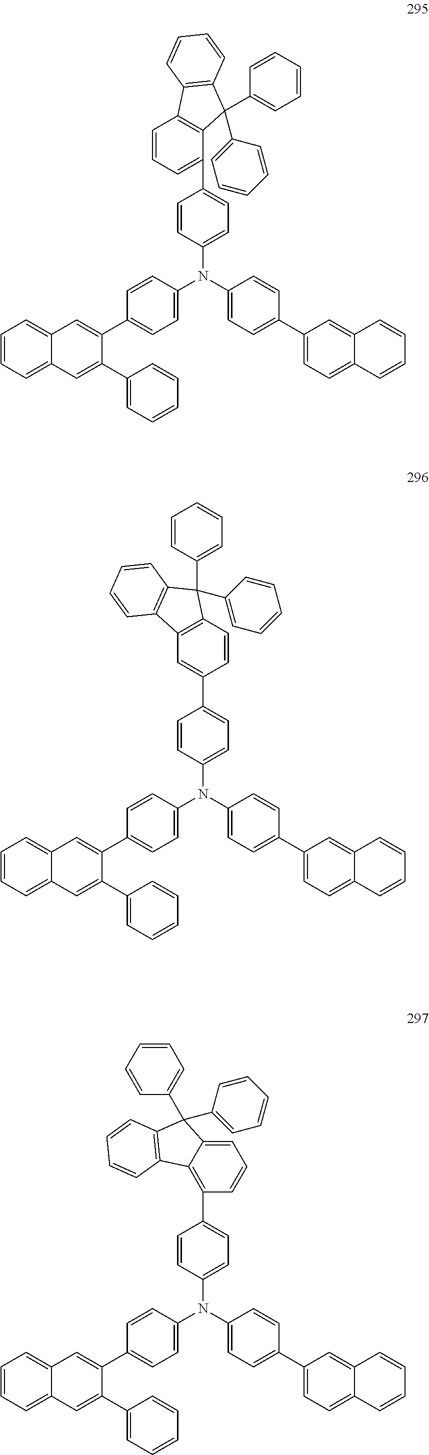 [SMILES: c1ccc(-c2cc3ccccc3cc2-c2ccc(N(c3ccc(-c4ccc5ccccc5c4)cc3)c3ccc(-c4cccc5c4C(c4ccccc4)(c4ccccc4)c4ccccc4-5)cc3)cc2)cc1 ; c1ccc(-c2cc3ccccc3cc2-c2ccc(N(c3ccc(-c4ccc5c(c4)-c4ccccc4C5(c4ccccc4)c4ccccc4)cc3)c3ccc(-c4ccc5ccccc5c4)cc3)cc2)cc1 ; c1ccc(-c2cc3ccccc3cc2-c2ccc(N(c3ccc(-c4ccc5ccccc5c4)cc3)c3ccc(-c4cccc5c4-c4ccccc4C5(c4ccccc4)c4ccccc4)cc3)cc2)cc1]

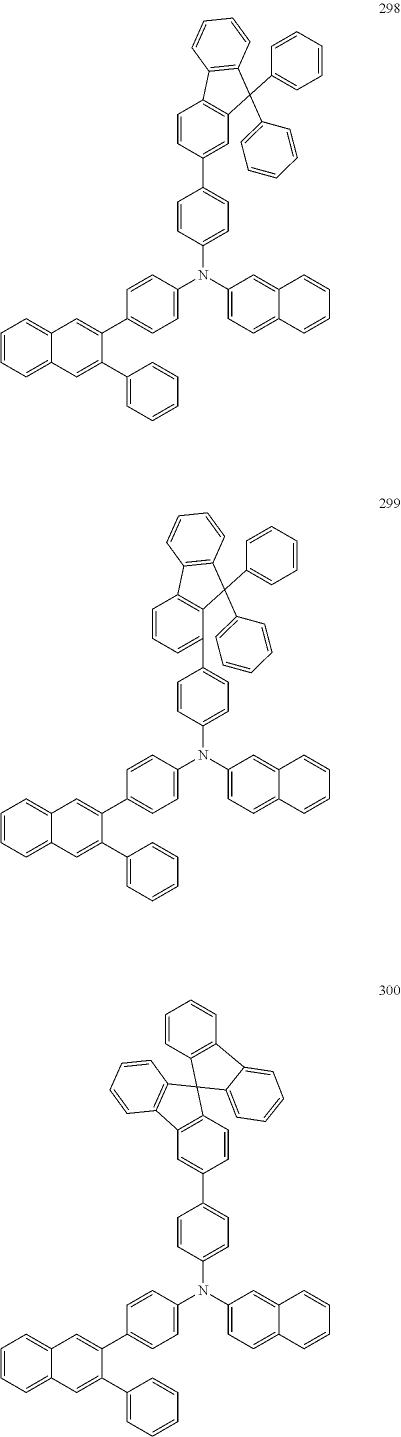 [SMILES: c1ccc(-c2cc3ccccc3cc2-c2ccc(N(c3ccc(-c4ccc5c(c4)C(c4ccccc4)(c4ccccc4)c4ccccc4-5)cc3)c3ccc4ccccc4c3)cc2)cc1 ; c1ccc(-c2cc3ccccc3cc2-c2ccc(N(c3ccc(-c4cccc5c4C(c4ccccc4)(c4ccccc4)c4ccccc4-5)cc3)c3ccc4ccccc4c3)cc2)cc1 ; c1ccc(-c2cc3ccccc3cc2-c2ccc(N(c3ccc(-c4ccc5c(c4)-c4ccccc4C54c5ccccc5-c5ccccc54)cc3)c3ccc4ccccc4c3)cc2)cc1]

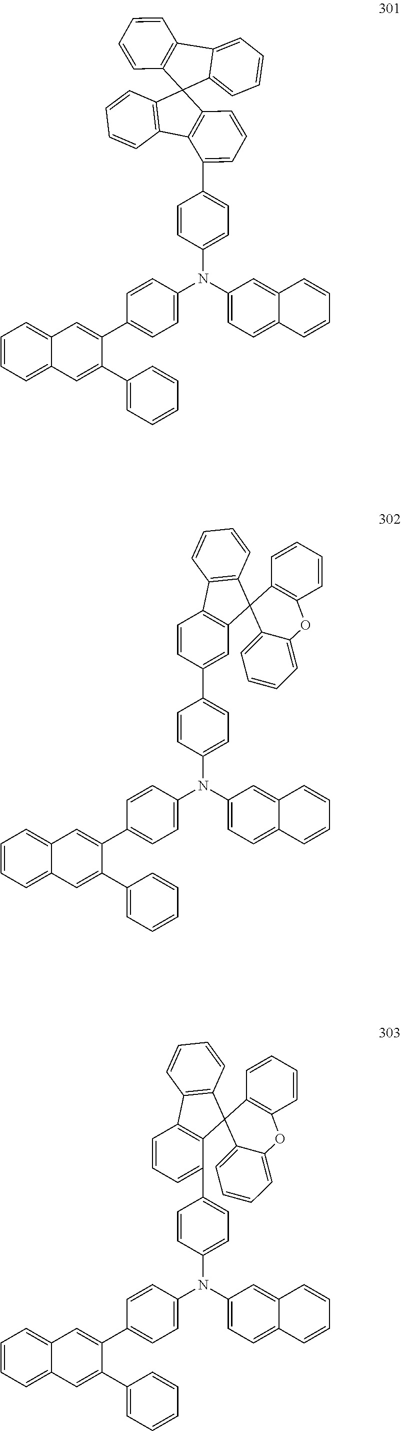 [SMILES: c1ccc(-c2cc3ccccc3cc2-c2ccc(N(c3ccc(-c4cccc5c4-c4ccccc4C54c5ccccc5-c5ccccc54)cc3)c3ccc4ccccc4c3)cc2)cc1 ; c1ccc(-c2cc3ccccc3cc2-c2ccc(N(c3ccc(-c4ccc5c(c4)C4(c6ccccc6Oc6ccccc64)c4ccccc4-5)cc3)c3ccc4ccccc4c3)cc2)cc1 ; c1ccc(-c2cc3ccccc3cc2-c2ccc(N(c3ccc(-c4cccc5c4C4(c6ccccc6Oc6ccccc64)c4ccccc4-5)cc3)c3ccc4ccccc4c3)cc2)cc1]

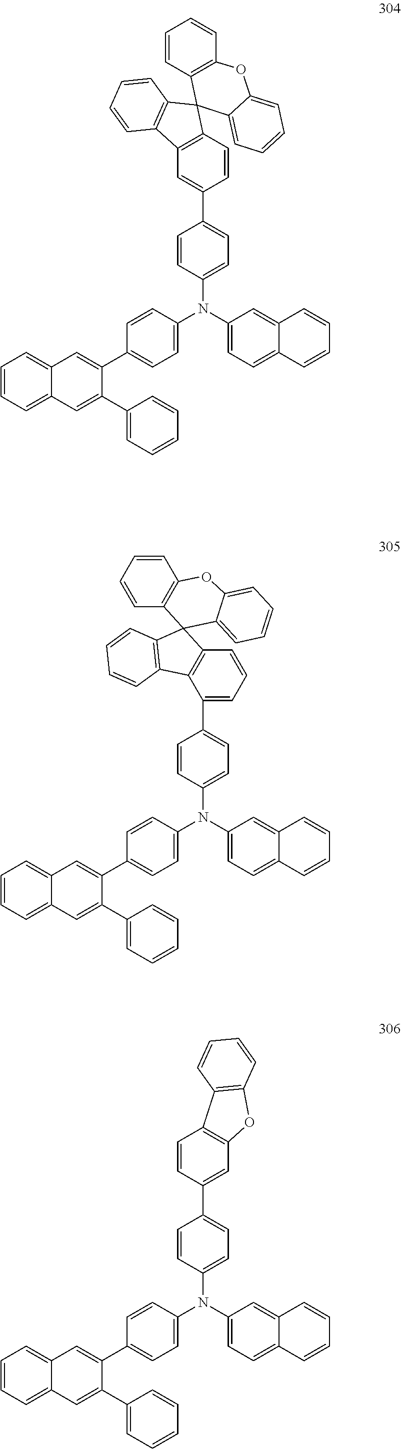 [SMILES: c1ccc(-c2cc3ccccc3cc2-c2ccc(N(c3ccc(-c4ccc5c(c4)-c4ccccc4C54c5ccccc5Oc5ccccc54)cc3)c3ccc4ccccc4c3)cc2)cc1 ; c1ccc(-c2cc3ccccc3cc2-c2ccc(N(c3ccc(-c4cccc5c4-c4ccccc4C54c5ccccc5Oc5ccccc54)cc3)c3ccc4ccccc4c3)cc2)cc1 ; c1ccc(-c2cc3ccccc3cc2-c2ccc(N(c3ccc(-c4ccc5c(c4)oc4ccccc45)cc3)c3ccc4ccccc4c3)cc2)cc1]

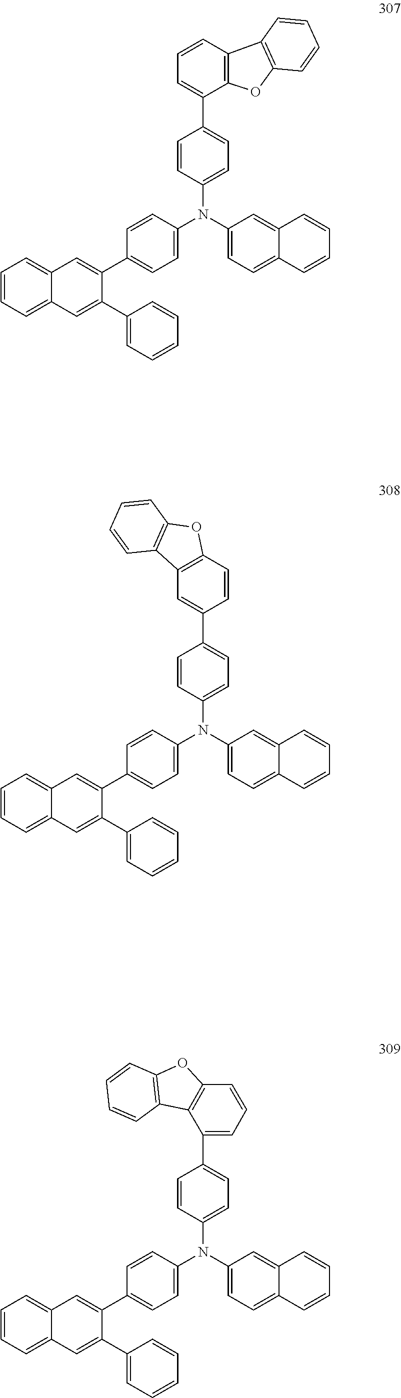 [SMILES: c1ccc(-c2cc3ccccc3cc2-c2ccc(N(c3ccc(-c4cccc5c4oc4ccccc45)cc3)c3ccc4ccccc4c3)cc2)cc1 ; c1ccc(-c2cc3ccccc3cc2-c2ccc(N(c3ccc(-c4ccc5oc6ccccc6c5c4)cc3)c3ccc4ccccc4c3)cc2)cc1 ; c1ccc(-c2cc3ccccc3cc2-c2ccc(N(c3ccc(-c4cccc5oc6ccccc6c45)cc3)c3ccc4ccccc4c3)cc2)cc1]

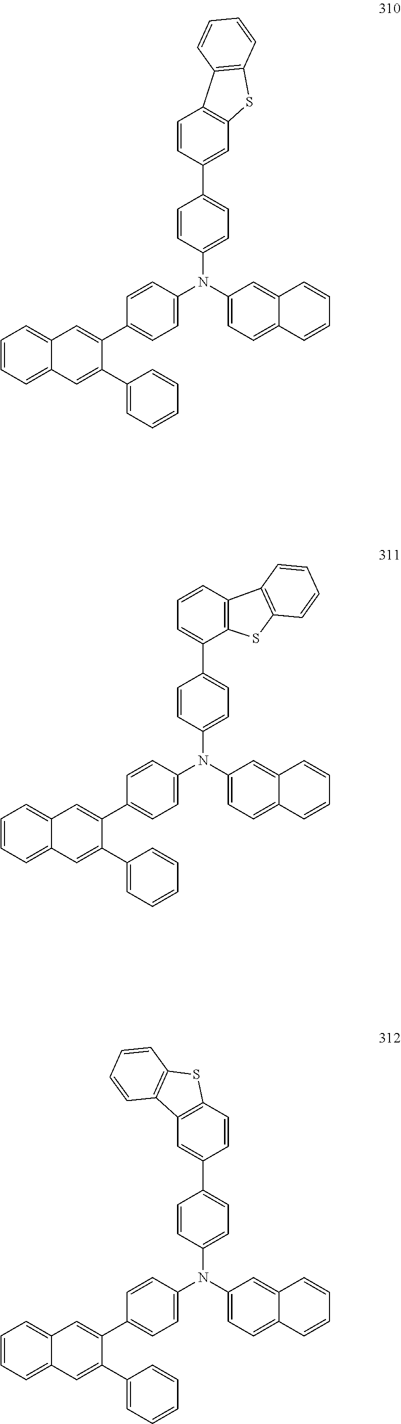 [SMILES: c1ccc(-c2cc3ccccc3cc2-c2ccc(N(c3ccc(-c4ccc5c(c4)sc4ccccc45)cc3)c3ccc4ccccc4c3)cc2)cc1 ; c1ccc(-c2cc3ccccc3cc2-c2ccc(N(c3ccc(-c4cccc5c4sc4ccccc45)cc3)c3ccc4ccccc4c3)cc2)cc1 ; c1ccc(-c2cc3ccccc3cc2-c2ccc(N(c3ccc(-c4ccc5sc6ccccc6c5c4)cc3)c3ccc4ccccc4c3)cc2)cc1]

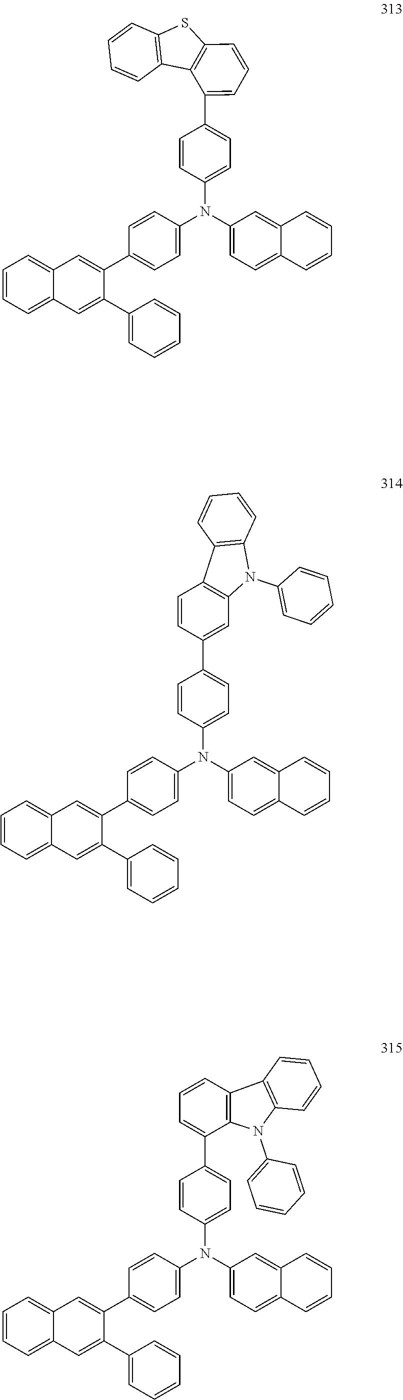 [SMILES: c1ccc(-c2cc3ccccc3cc2-c2ccc(N(c3ccc(-c4cccc5sc6ccccc6c45)cc3)c3ccc4ccccc4c3)cc2)cc1 ; c1ccc(-c2cc3ccccc3cc2-c2ccc(N(c3ccc(-c4ccc5c6ccccc6n(-c6ccccc6)c5c4)cc3)c3ccc4ccccc4c3)cc2)cc1 ; c1ccc(-c2cc3ccccc3cc2-c2ccc(N(c3ccc(-c4cccc5c6ccccc6n(-c6ccccc6)c45)cc3)c3ccc4ccccc4c3)cc2)cc1]

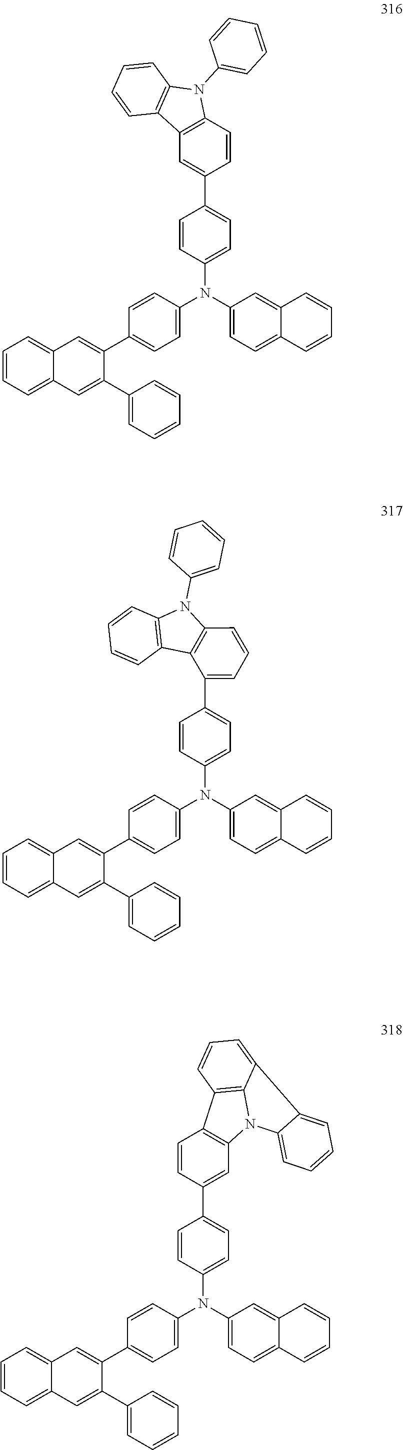 [SMILES: c1ccc(-c2cc3ccccc3cc2-c2ccc(N(c3ccc(-c4ccc5c(c4)c4ccccc4n5-c4ccccc4)cc3)c3ccc4ccccc4c3)cc2)cc1 ; c1ccc(-c2cc3ccccc3cc2-c2ccc(N(c3ccc(-c4cccc5c4c4ccccc4n5-c4ccccc4)cc3)c3ccc4ccccc4c3)cc2)cc1 ; c1ccc(-c2cc3ccccc3cc2-c2ccc(N(c3ccc(-c4ccc5c6cccc7c8ccccc8n(c5c4)c76)cc3)c3ccc4ccccc4c3)cc2)cc1]

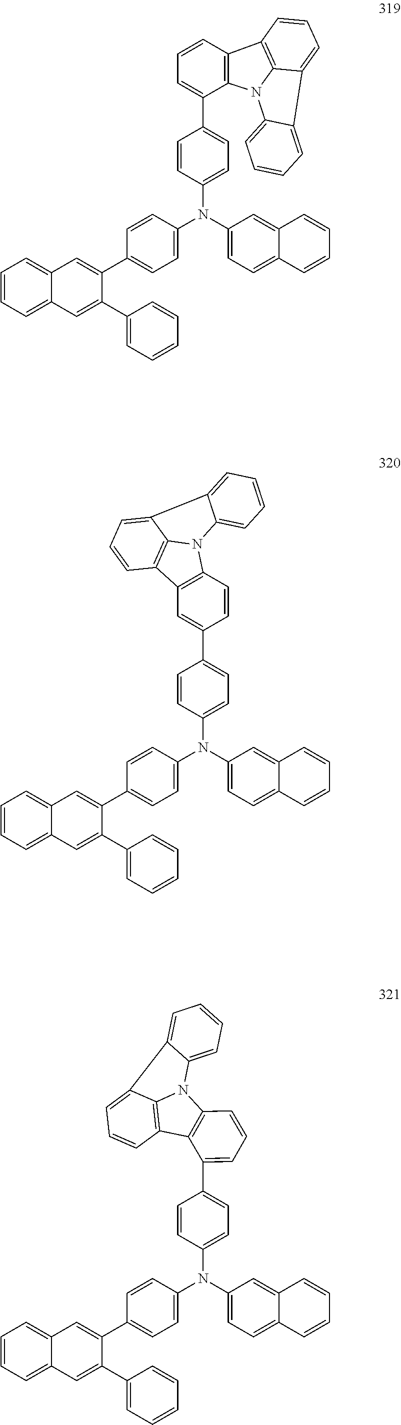 [SMILES: c1ccc(-c2cc3ccccc3cc2-c2ccc(N(c3ccc(-c4cccc5c6cccc7c8ccccc8n(c45)c76)cc3)c3ccc4ccccc4c3)cc2)cc1 ; c1ccc(-c2cc3ccccc3cc2-c2ccc(N(c3ccc(-c4ccc5c(c4)c4cccc6c7ccccc7n5c64)cc3)c3ccc4ccccc4c3)cc2)cc1 ; c1ccc(-c2cc3ccccc3cc2-c2ccc(N(c3ccc(-c4cccc5c4c4cccc6c7ccccc7n5c64)cc3)c3ccc4ccccc4c3)cc2)cc1]

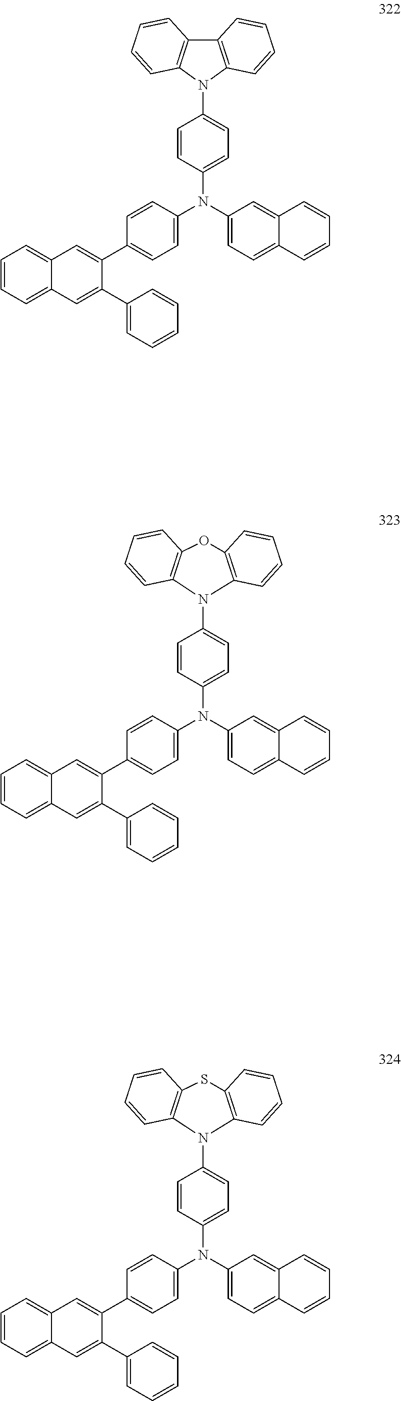 [SMILES: c1ccc(-c2cc3ccccc3cc2-c2ccc(N(c3ccc(-n4c5ccccc5c5ccccc54)cc3)c3ccc4ccccc4c3)cc2)cc1 ; c1ccc(-c2cc3ccccc3cc2-c2ccc(N(c3ccc(N4c5ccccc5Oc5ccccc54)cc3)c3ccc4ccccc4c3)cc2)cc1 ; c1ccc(-c2cc3ccccc3cc2-c2ccc(N(c3ccc(N4c5ccccc5Sc5ccccc54)cc3)c3ccc4ccccc4c3)cc2)cc1]

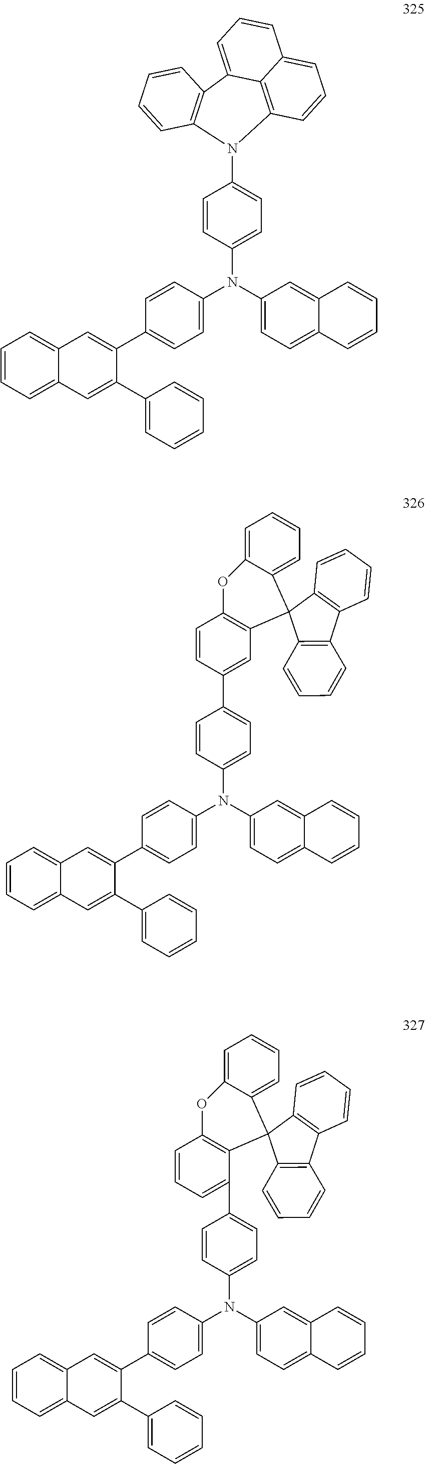 [SMILES: c1ccc(-c2cc3ccccc3cc2-c2ccc(N(c3ccc(N4c5ccccc5-c5cccc6cccc4c56)cc3)c3ccc4ccccc4c3)cc2)cc1 ; c1ccc(-c2cc3ccccc3cc2-c2ccc(N(c3ccc(-c4ccc5c(c4)C4(c6ccccc6O5)c5ccccc5-c5ccccc54)cc3)c3ccc4ccccc4c3)cc2)cc1 ; c1ccc(-c2cc3ccccc3cc2-c2ccc(N(c3ccc(-c4cccc5c4C4(c6ccccc6O5)c5ccccc5-c5ccccc54)cc3)c3ccc4ccccc4c3)cc2)cc1]

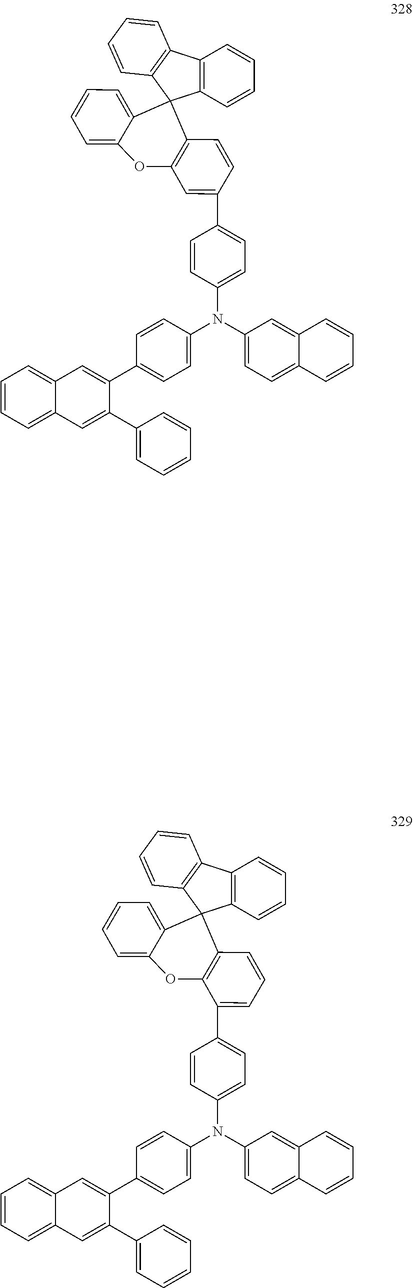 [SMILES: c1ccc(-c2cc3ccccc3cc2-c2ccc(N(c3ccc(-c4ccc5c(c4)Oc4ccccc4C54c5ccccc5-c5ccccc54)cc3)c3ccc4ccccc4c3)cc2)cc1 ; c1ccc(-c2cc3ccccc3cc2-c2ccc(N(c3ccc(-c4cccc5c4Oc4ccccc4C54c5ccccc5-c5ccccc54)cc3)c3ccc4ccccc4c3)cc2)cc1]

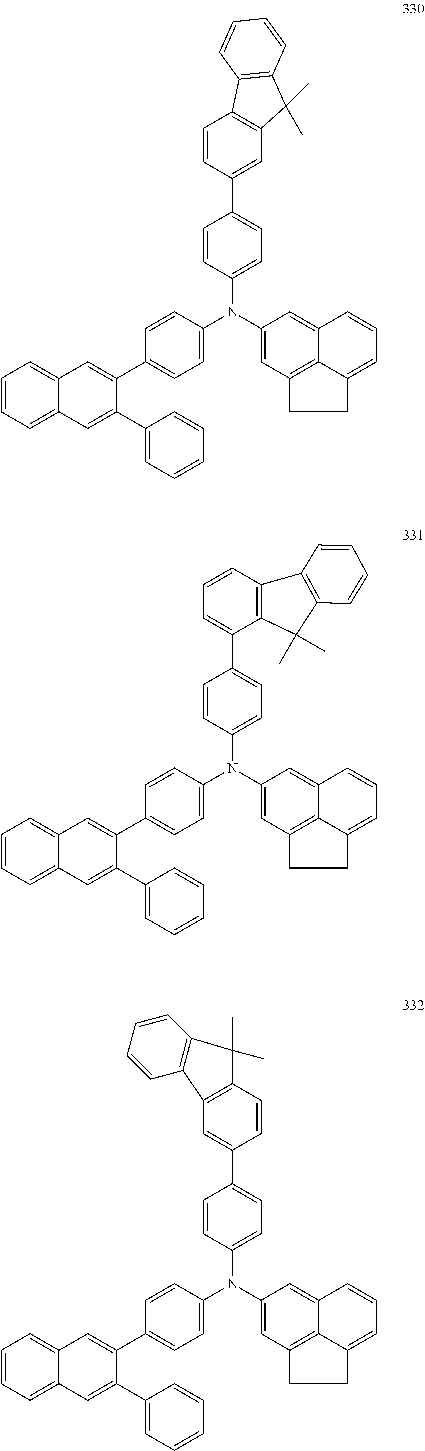 [SMILES: CC1(C)c2ccccc2-c2ccc(-c3ccc(N(c4ccc(-c5cc6ccccc6cc5-c5ccccc5)cc4)c4cc5c6c(cccc6c4)CC5)cc3)cc21 ; CC1(C)c2ccccc2-c2cccc(-c3ccc(N(c4ccc(-c5cc6ccccc6cc5-c5ccccc5)cc4)c4cc5c6c(cccc6c4)CC5)cc3)c21 ; CC1(C)c2ccccc2-c2cc(-c3ccc(N(c4ccc(-c5cc6ccccc6cc5-c5ccccc5)cc4)c4cc5c6c(cccc6c4)CC5)cc3)ccc21]

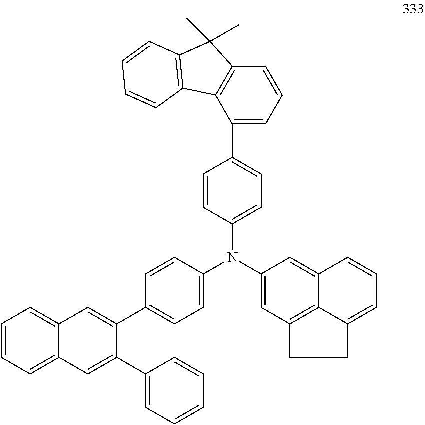 [SMILES: CC1(C)c2ccccc2-c2c(-c3ccc(N(c4ccc(-c5cc6ccccc6cc5-c5ccccc5)cc4)c4cc5c6c(cccc6c4)CC5)cc3)cccc21]

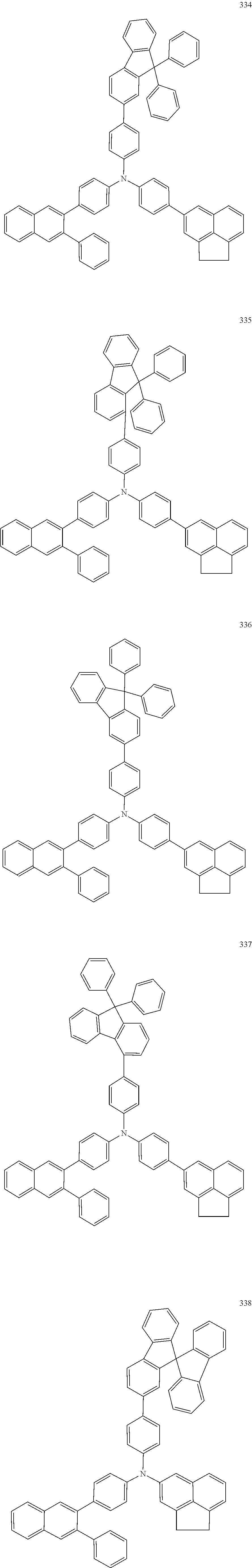

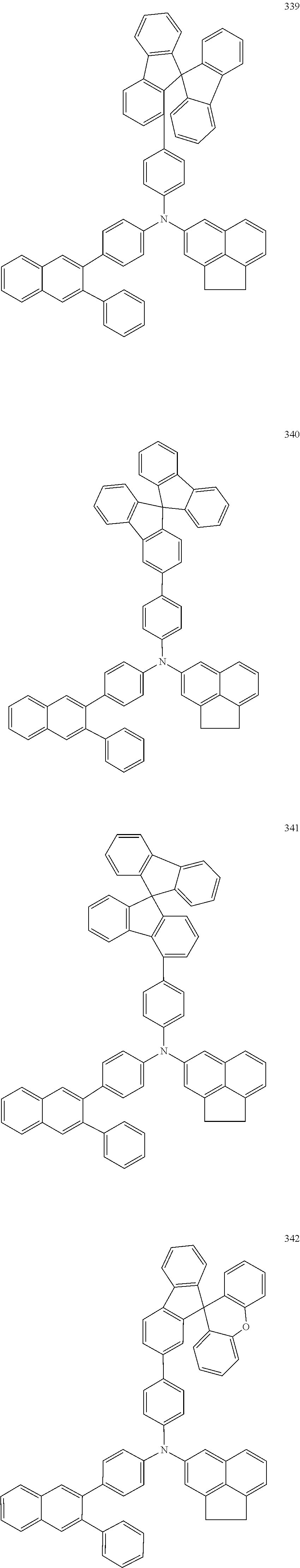 [SMILES: c1ccc(-c2cc3ccccc3cc2-c2ccc(N(c3ccc(-c4cccc5c4C4(c6ccccc6-c6ccccc64)c4ccccc4-5)cc3)c3cc4c5c(cccc5c3)CC4)cc2)cc1 ; c1ccc(-c2cc3ccccc3cc2-c2ccc(N(c3ccc(-c4ccc5c(c4)-c4ccccc4C54c5ccccc5-c5ccccc54)cc3)c3cc4c5c(cccc5c3)CC4)cc2)cc1 ; c1ccc(-c2cc3ccccc3cc2-c2ccc(N(c3ccc(-c4cccc5c4-c4ccccc4C54c5ccccc5-c5ccccc54)cc3)c3cc4c5c(cccc5c3)CC4)cc2)cc1 ; c1ccc(-c2cc3ccccc3cc2-c2ccc(N(c3ccc(-c4ccc5c(c4)C4(c6ccccc6Oc6ccccc64)c4ccccc4-5)cc3)c3cc4c5c(cccc5c3)CC4)cc2)cc1]

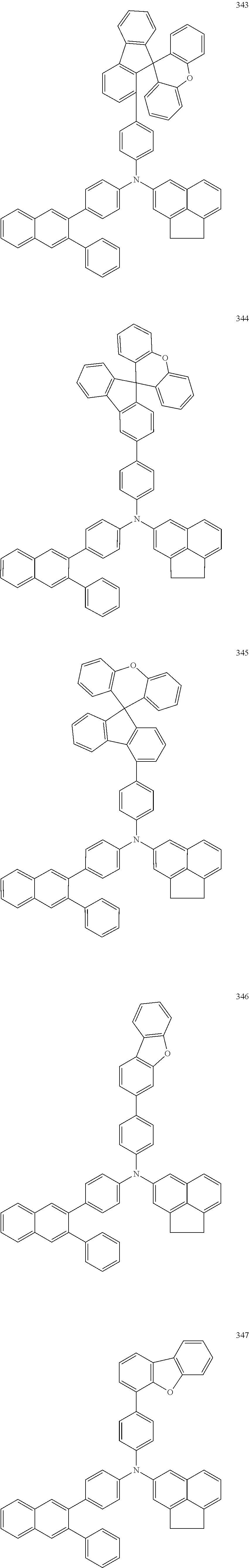

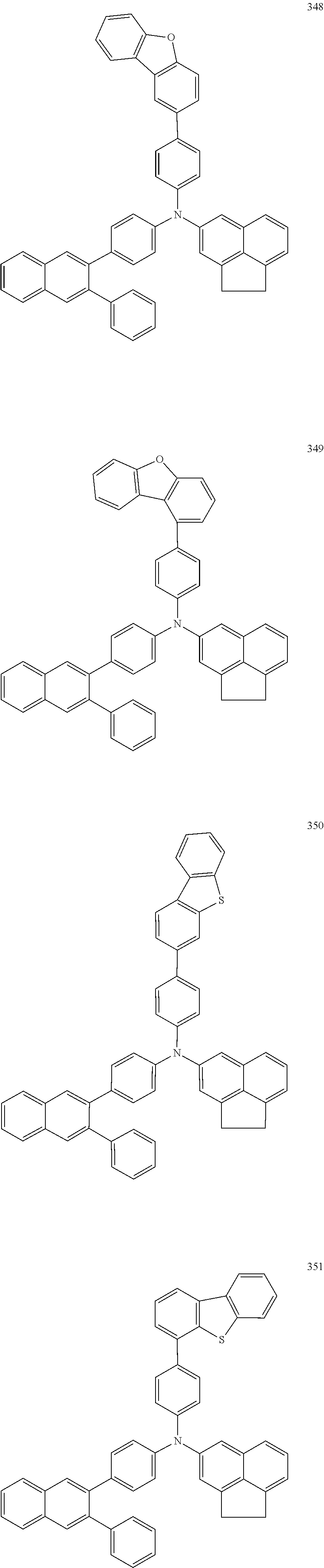 [SMILES: c1ccc(-c2cc3ccccc3cc2-c2ccc(N(c3ccc(-c4ccc5oc6ccccc6c5c4)cc3)c3cc4c5c(cccc5c3)CC4)cc2)cc1 ; c1ccc(-c2cc3ccccc3cc2-c2ccc(N(c3ccc(-c4cccc5oc6ccccc6c45)cc3)c3cc4c5c(cccc5c3)CC4)cc2)cc1 ; c1ccc(-c2cc3ccccc3cc2-c2ccc(N(c3ccc(-c4ccc5c(c4)sc4ccccc45)cc3)c3cc4c5c(cccc5c3)CC4)cc2)cc1 ; c1ccc(-c2cc3ccccc3cc2-c2ccc(N(c3ccc(-c4cccc5c4sc4ccccc45)cc3)c3cc4c5c(cccc5c3)CC4)cc2)cc1]

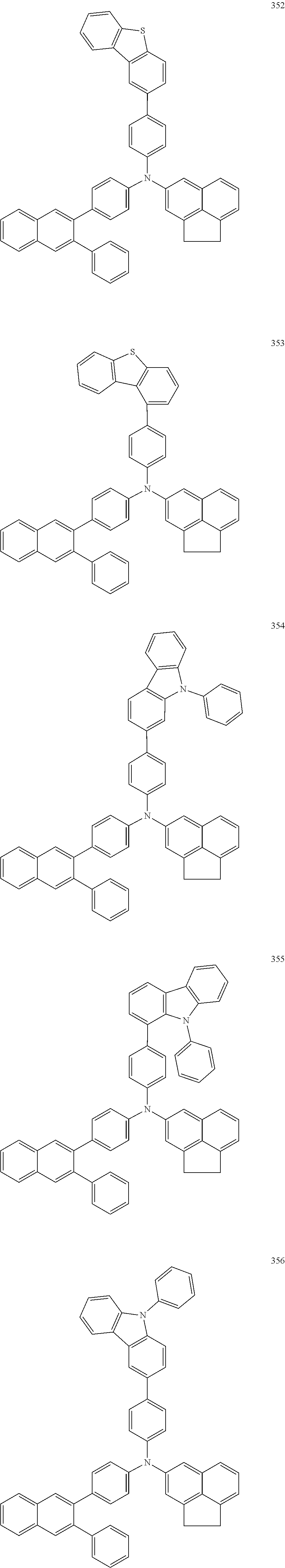 [SMILES: c1ccc(-c2cc3ccccc3cc2-c2ccc(N(c3ccc(-c4ccc5sc6ccccc6c5c4)cc3)c3cc4c5c(cccc5c3)CC4)cc2)cc1 ; c1ccc(-c2cc3ccccc3cc2-c2ccc(N(c3ccc(-c4cccc5sc6ccccc6c45)cc3)c3cc4c5c(cccc5c3)CC4)cc2)cc1 ; c1ccc(-c2cc3ccccc3cc2-c2ccc(N(c3ccc(-c4ccc5c6ccccc6n(-c6ccccc6)c5c4)cc3)c3cc4c5c(cccc5c3)CC4)cc2)cc1 ; c1ccc(-c2cc3ccccc3cc2-c2ccc(N(c3ccc(-c4cccc5c6ccccc6n(-c6ccccc6)c45)cc3)c3cc4c5c(cccc5c3)CC4)cc2)cc1 ; c1ccc(-c2cc3ccccc3cc2-c2ccc(N(c3ccc(-c4ccc5c(c4)c4ccccc4n5-c4ccccc4)cc3)c3cc4c5c(cccc5c3)CC4)cc2)cc1]

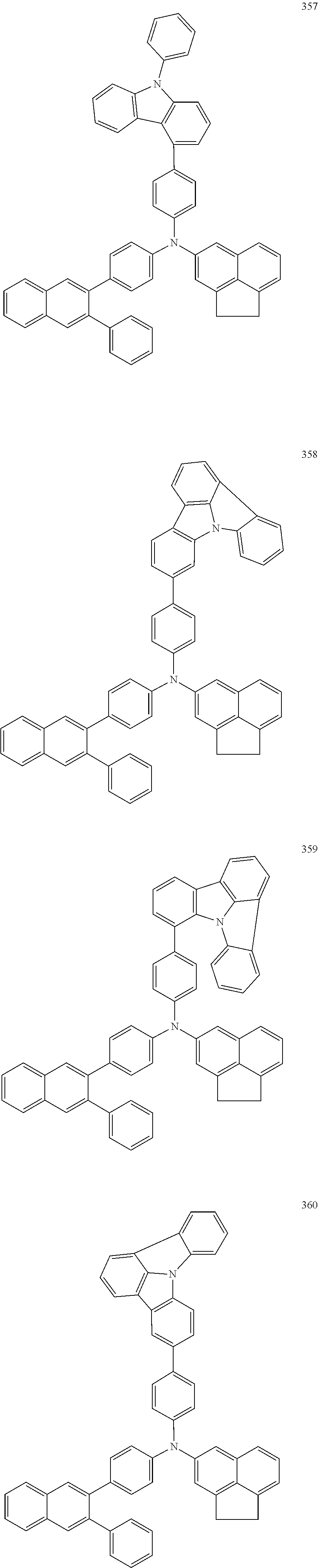 [SMILES: c1ccc(-c2cc3ccccc3cc2-c2ccc(N(c3ccc(-c4cccc5c4c4ccccc4n5-c4ccccc4)cc3)c3cc4c5c(cccc5c3)CC4)cc2)cc1 ; c1ccc(-c2cc3ccccc3cc2-c2ccc(N(c3ccc(-c4ccc5c6cccc7c8ccccc8n(c5c4)c76)cc3)c3cc4c5c(cccc5c3)CC4)cc2)cc1 ; c1ccc(-c2cc3ccccc3cc2-c2ccc(N(c3ccc(-c4cccc5c6cccc7c8ccccc8n(c45)c76)cc3)c3cc4c5c(cccc5c3)CC4)cc2)cc1 ; c1ccc(-c2cc3ccccc3cc2-c2ccc(N(c3ccc(-c4ccc5c(c4)c4cccc6c7ccccc7n5c64)cc3)c3cc4c5c(cccc5c3)CC4)cc2)cc1]

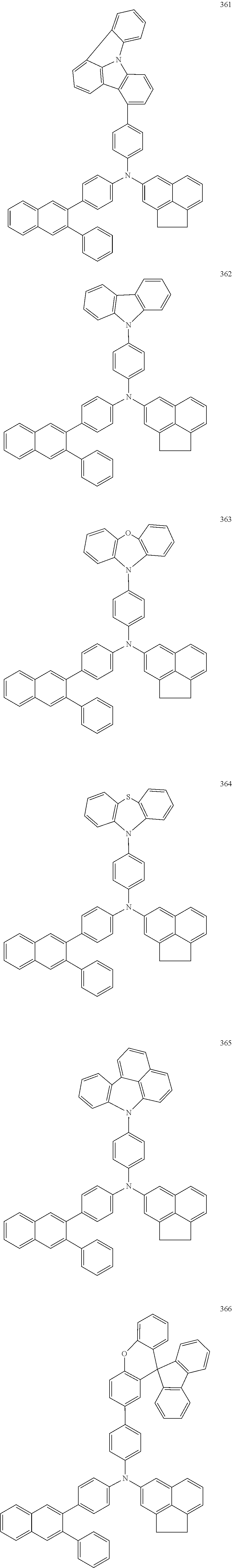

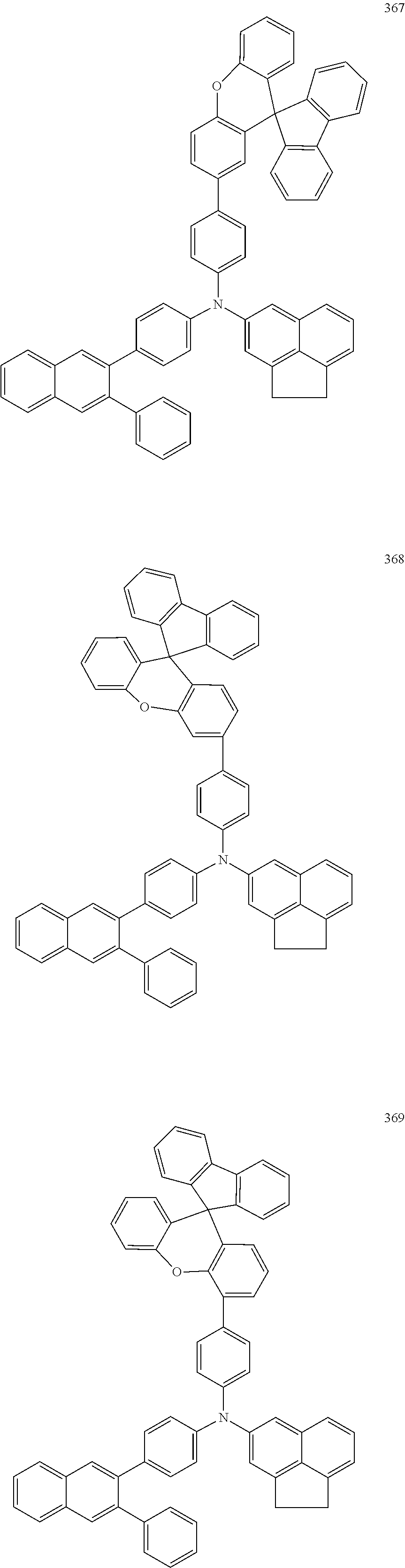 [SMILES: c1ccc(-c2cc3ccccc3cc2-c2ccc(N(c3ccc(-c4ccc5c(c4)C4(c6ccccc6O5)c5ccccc5-c5ccccc54)cc3)c3cc4c5c(cccc5c3)CC4)cc2)cc1 ; c1ccc(-c2cc3ccccc3cc2-c2ccc(N(c3ccc(-c4ccc5c(c4)Oc4ccccc4C54c5ccccc5-c5ccccc54)cc3)c3cc4c5c(cccc5c3)CC4)cc2)cc1 ; c1ccc(-c2cc3ccccc3cc2-c2ccc(N(c3ccc(-c4cccc5c4Oc4ccccc4C54c5ccccc5-c5ccccc54)cc3)c3cc4c5c(cccc5c3)CC4)cc2)cc1]

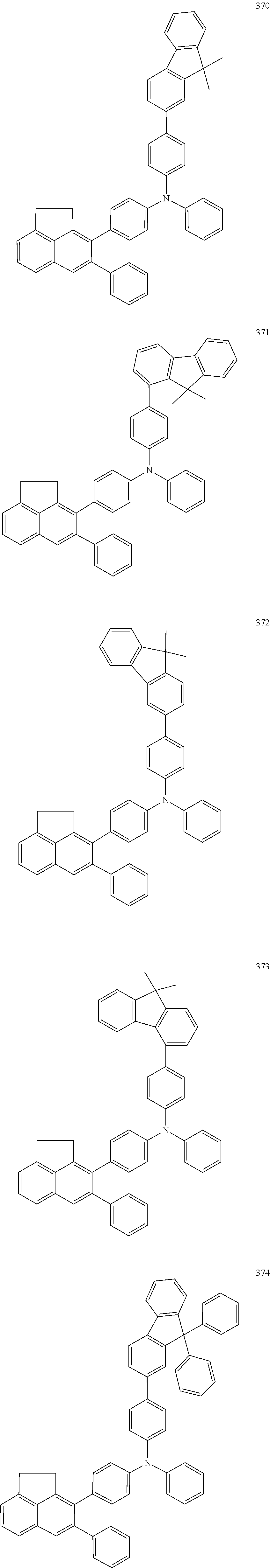 [SMILES: CC1(C)c2ccccc2-c2ccc(-c3ccc(N(c4ccccc4)c4ccc(-c5c(-c6ccccc6)cc6cccc7c6c5CC7)cc4)cc3)cc21 ; CC1(C)c2ccccc2-c2cccc(-c3ccc(N(c4ccccc4)c4ccc(-c5c(-c6ccccc6)cc6cccc7c6c5CC7)cc4)cc3)c21 ; CC1(C)c2ccccc2-c2cc(-c3ccc(N(c4ccccc4)c4ccc(-c5c(-c6ccccc6)cc6cccc7c6c5CC7)cc4)cc3)ccc21 ; CC1(C)c2ccccc2-c2c(-c3ccc(N(c4ccccc4)c4ccc(-c5c(-c6ccccc6)cc6cccc7c6c5CC7)cc4)cc3)cccc21 ; c1ccc(-c2cc3cccc4c3c(c2-c2ccc(N(c3ccccc3)c3ccc(-c5ccc6c(c5)C(c5ccccc5)(c5ccccc5)c5ccccc5-6)cc3)cc2)CC4)cc1]

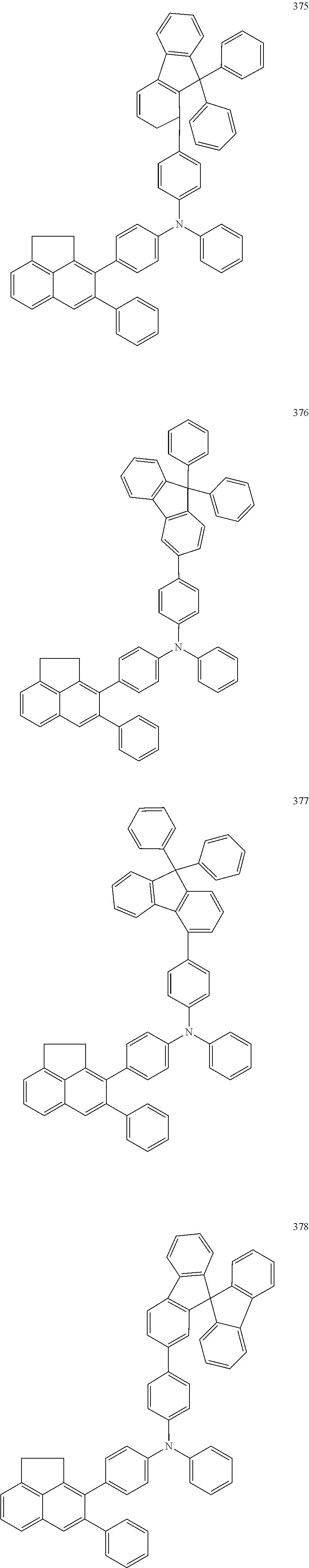 [SMILES: C1=CC2=C(CC(c3ccc(N(c4ccccc4)c4ccc(-c5c(-c6ccccc6)cc6cccc7c6c5CC7)cc4)cc3)C1)C(c1ccccc1)(c1ccccc1)c1ccccc12 ; c1ccc(-c2cc3cccc4c3c(c2-c2ccc(N(c3ccccc3)c3ccc(-c5ccc6c(c5)-c5ccccc5C6(c5ccccc5)c5ccccc5)cc3)cc2)CC4)cc1 ; c1ccc(-c2cc3cccc4c3c(c2-c2ccc(N(c3ccccc3)c3ccc(-c5cccc6c5-c5ccccc5C6(c5ccccc5)c5ccccc5)cc3)cc2)CC4)cc1 ; c1ccc(-c2cc3cccc4c3c(c2-c2ccc(N(c3ccccc3)c3ccc(-c5ccc6c(c5)C5(c7ccccc7-c7ccccc75)c5ccccc5-6)cc3)cc2)CC4)cc1]

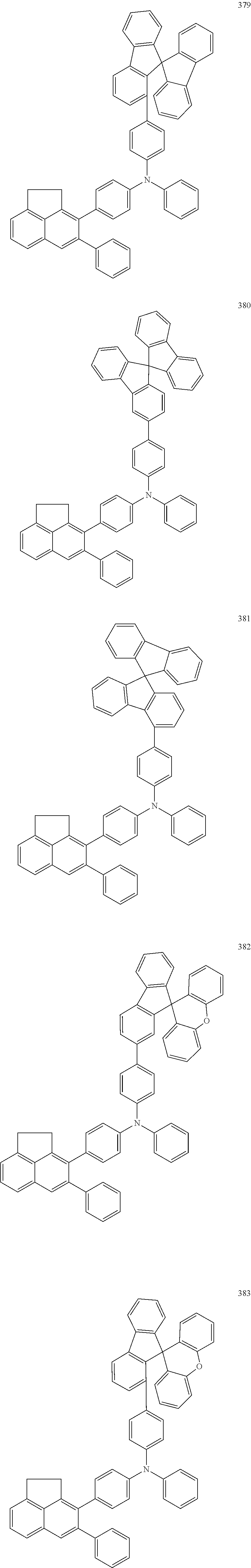

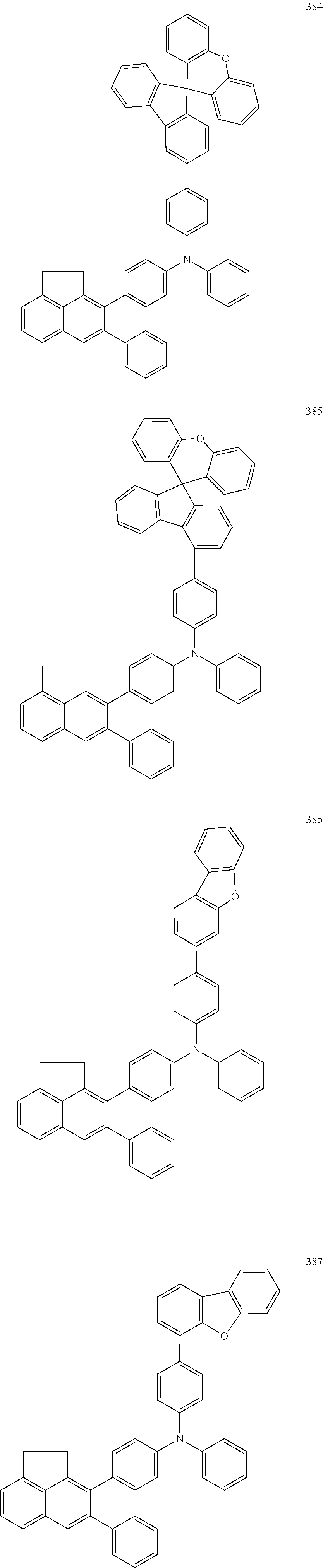 [SMILES: c1ccc(-c2cc3cccc4c3c(c2-c2ccc(N(c3ccccc3)c3ccc(-c5ccc6c(c5)-c5ccccc5C65c6ccccc6Oc6ccccc65)cc3)cc2)CC4)cc1 ; c1ccc(-c2cc3cccc4c3c(c2-c2ccc(N(c3ccccc3)c3ccc(-c5cccc6c5-c5ccccc5C65c6ccccc6Oc6ccccc65)cc3)cc2)CC4)cc1 ; c1ccc(-c2cc3cccc4c3c(c2-c2ccc(N(c3ccccc3)c3ccc(-c5ccc6c(c5)oc5ccccc56)cc3)cc2)CC4)cc1 ; c1ccc(-c2cc3cccc4c3c(c2-c2ccc(N(c3ccccc3)c3ccc(-c5cccc6c5oc5ccccc56)cc3)cc2)CC4)cc1]

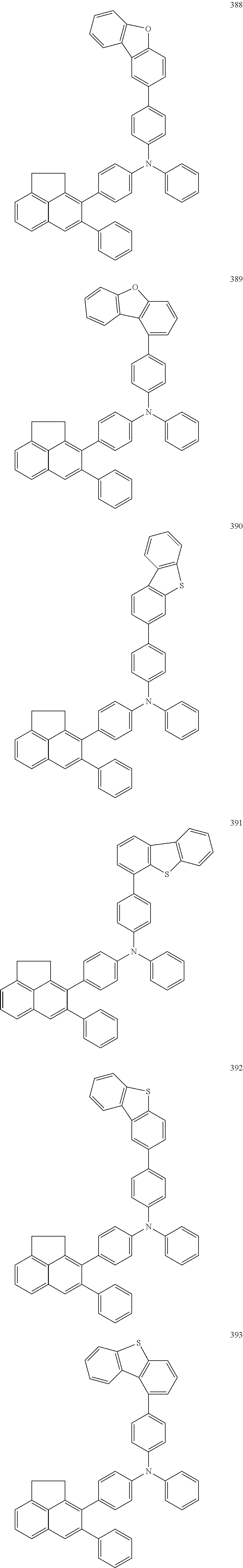

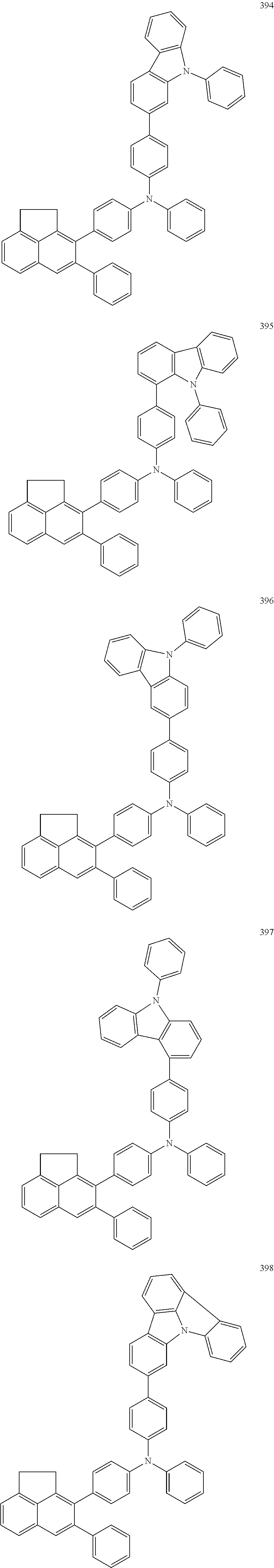 [SMILES: c1ccc(-c2cc3cccc4c3c(c2-c2ccc(N(c3ccccc3)c3ccc(-c5ccc6c7ccccc7n(-c7ccccc7)c6c5)cc3)cc2)CC4)cc1 ; c1ccc(-c2cc3cccc4c3c(c2-c2ccc(N(c3ccccc3)c3ccc(-c5cccc6c7ccccc7n(-c7ccccc7)c56)cc3)cc2)CC4)cc1 ; c1ccc(-c2cc3cccc4c3c(c2-c2ccc(N(c3ccccc3)c3ccc(-c5ccc6c(c5)c5ccccc5n6-c5ccccc5)cc3)cc2)CC4)cc1 ; c1ccc(-c2cc3cccc4c3c(c2-c2ccc(N(c3ccccc3)c3ccc(-c5cccc6c5c5ccccc5n6-c5ccccc5)cc3)cc2)CC4)cc1 ; c1ccc(-c2cc3cccc4c3c(c2-c2ccc(N(c3ccccc3)c3ccc(-c5ccc6c7cccc8c9ccccc9n(c6c5)c87)cc3)cc2)CC4)cc1]

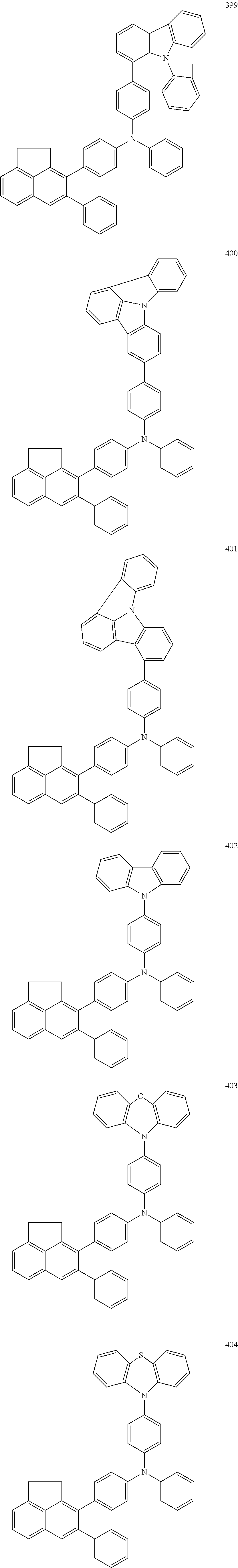

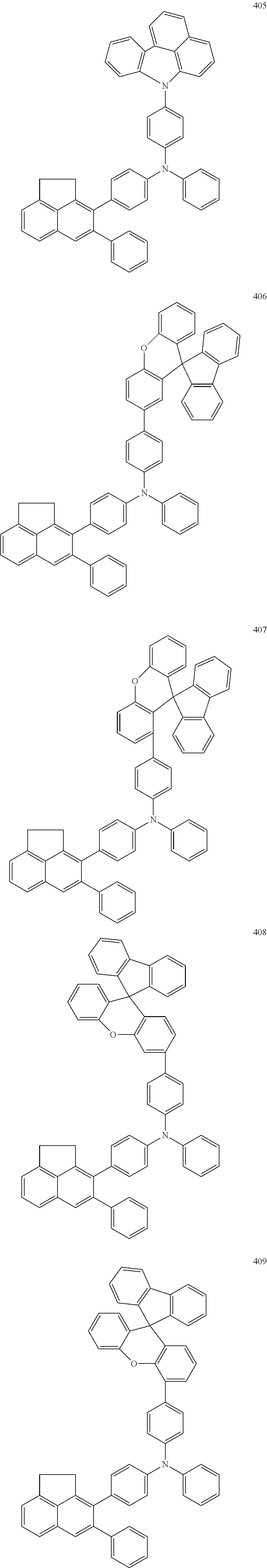

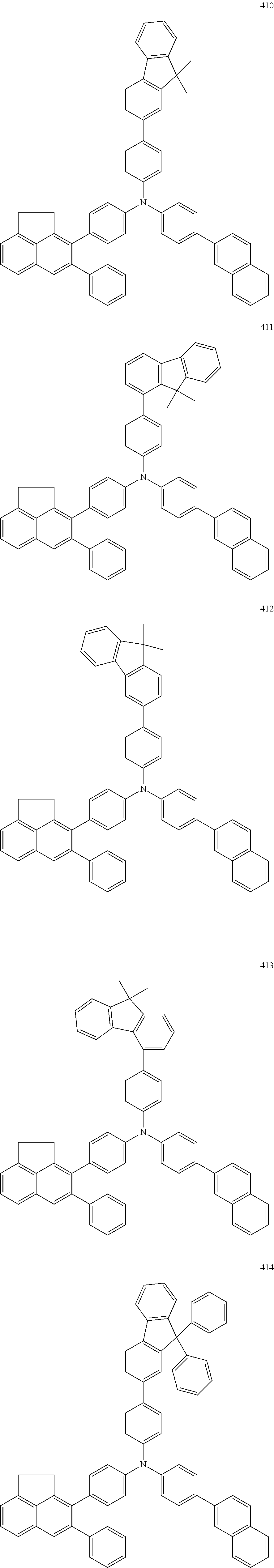

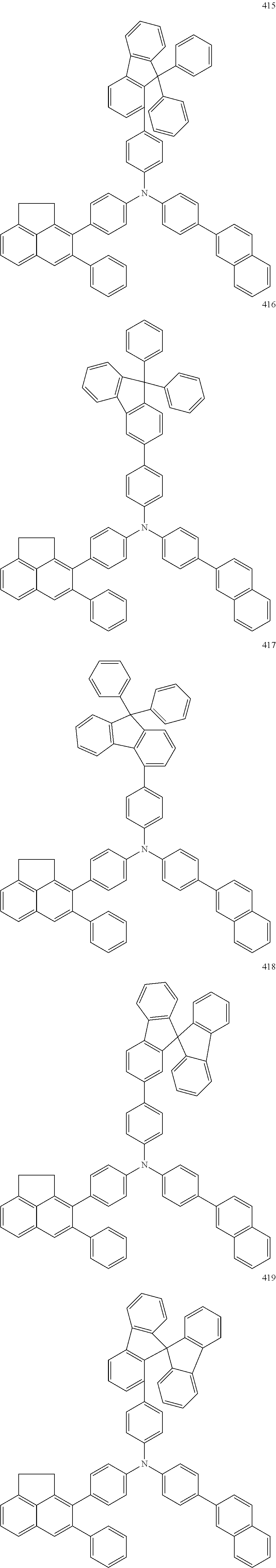

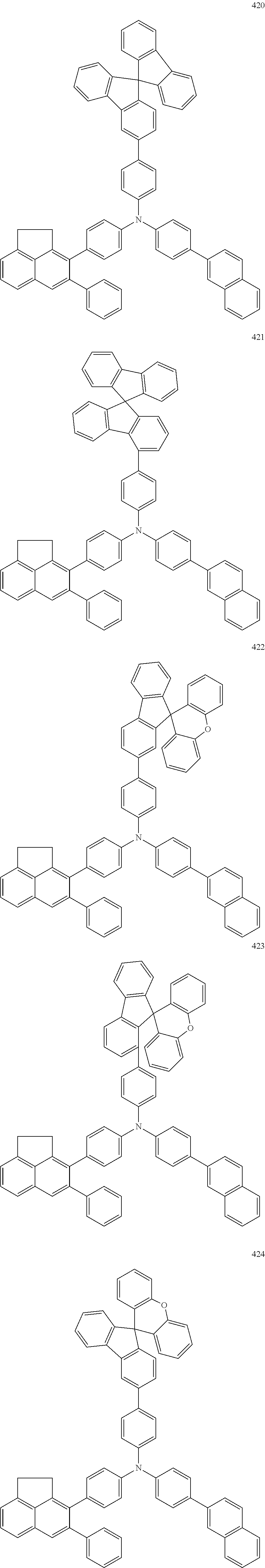

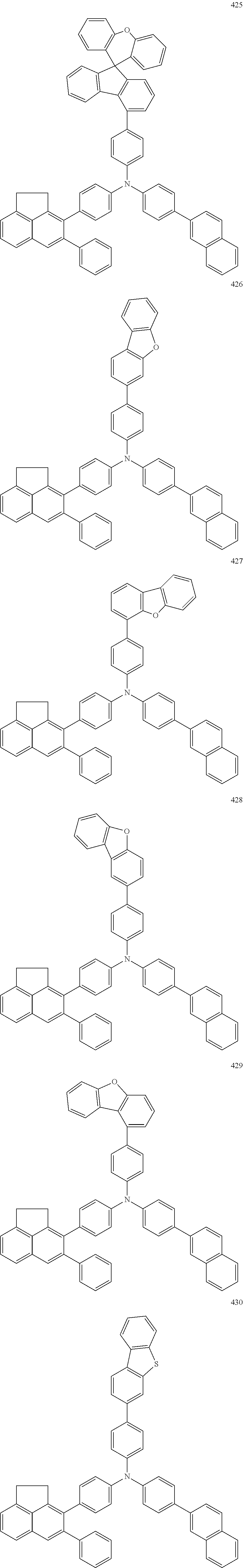

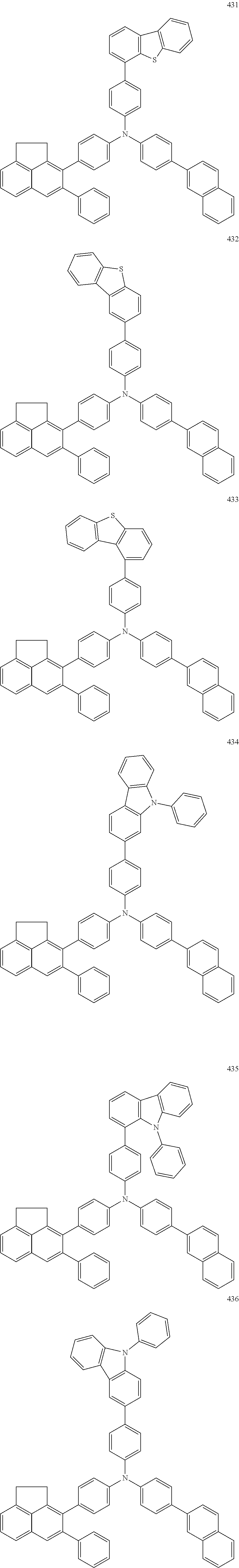

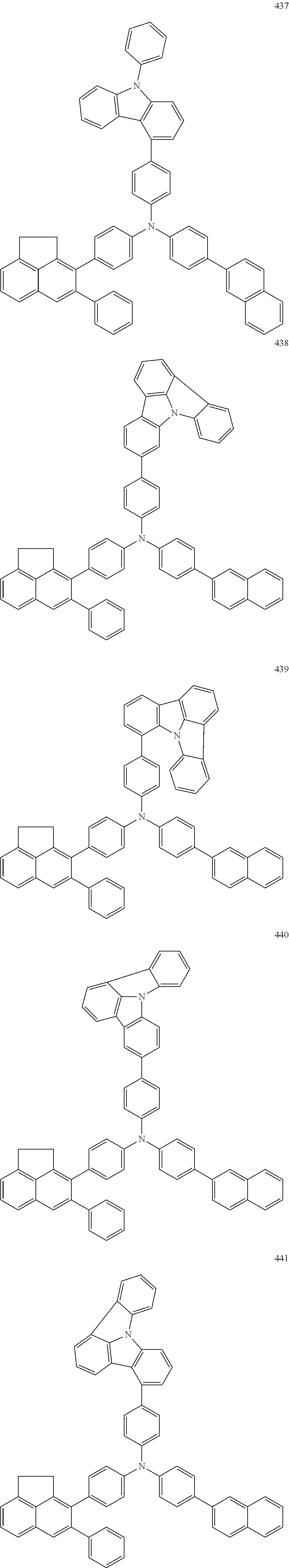

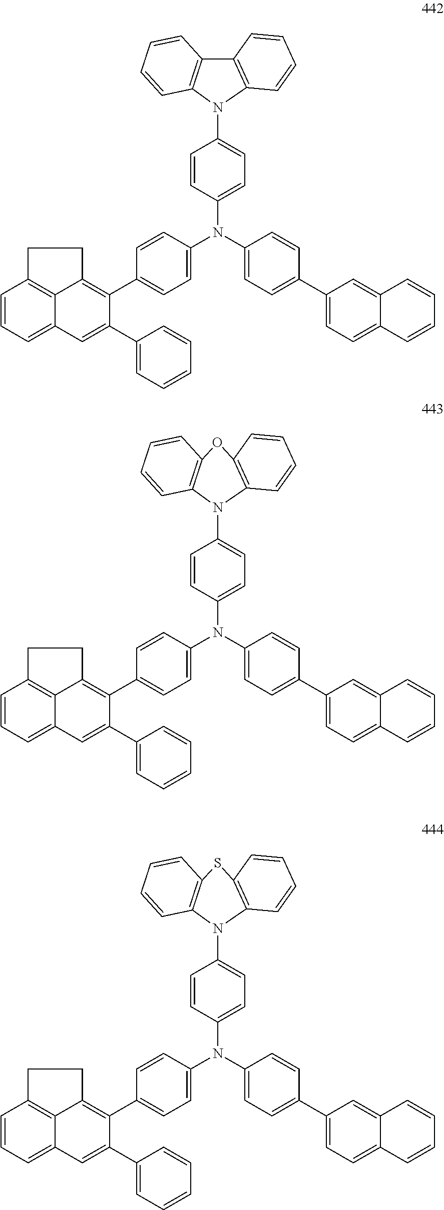 [SMILES: c1ccc(-c2cc3cccc4c3c(c2-c2ccc(N(c3ccc(-c5ccc6ccccc6c5)cc3)c3ccc(-n5c6ccccc6c6ccccc65)cc3)cc2)CC4)cc1 ; c1ccc(-c2cc3cccc4c3c(c2-c2ccc(N(c3ccc(-c5ccc6ccccc6c5)cc3)c3ccc(N5c6ccccc6Oc6ccccc65)cc3)cc2)CC4)cc1 ; c1ccc(-c2cc3cccc4c3c(c2-c2ccc(N(c3ccc(-c5ccc6ccccc6c5)cc3)c3ccc(N5c6ccccc6Sc6ccccc65)cc3)cc2)CC4)cc1]

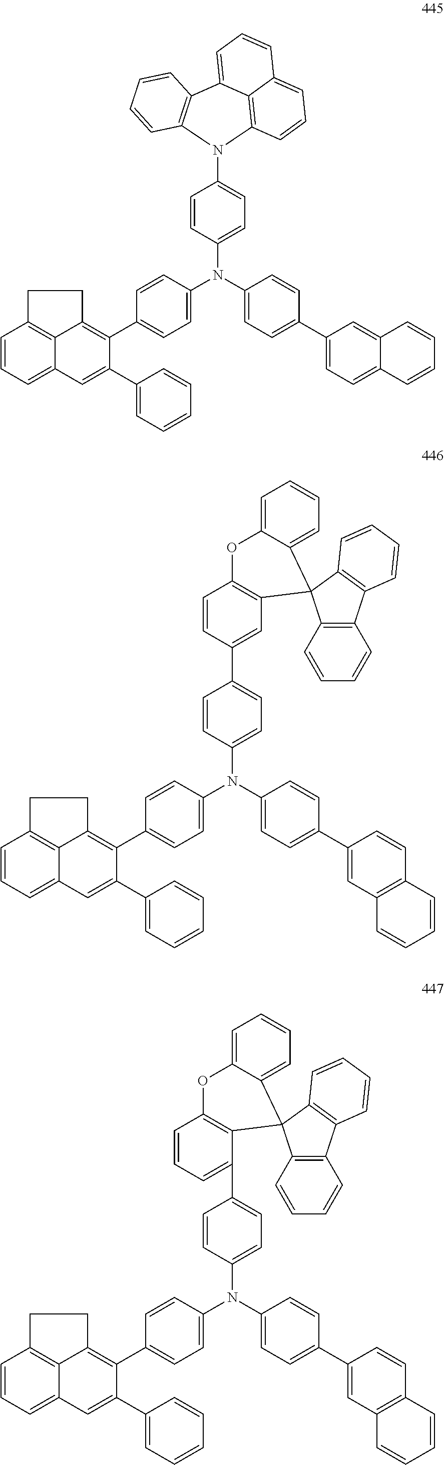 [SMILES: c1ccc(-c2cc3cccc4c3c(c2-c2ccc(N(c3ccc(-c5ccc6ccccc6c5)cc3)c3ccc(N5c6ccccc6-c6cccc7cccc5c67)cc3)cc2)CC4)cc1 ; c1ccc(-c2cc3cccc4c3c(c2-c2ccc(N(c3ccc(-c5ccc6c(c5)C5(c7ccccc7O6)c6ccccc6-c6ccccc65)cc3)c3ccc(-c5ccc6ccccc6c5)cc3)cc2)CC4)cc1 ; c1ccc(-c2cc3cccc4c3c(c2-c2ccc(N(c3ccc(-c5ccc6ccccc6c5)cc3)c3ccc(-c5cccc6c5C5(c7ccccc7O6)c6ccccc6-c6ccccc65)cc3)cc2)CC4)cc1]

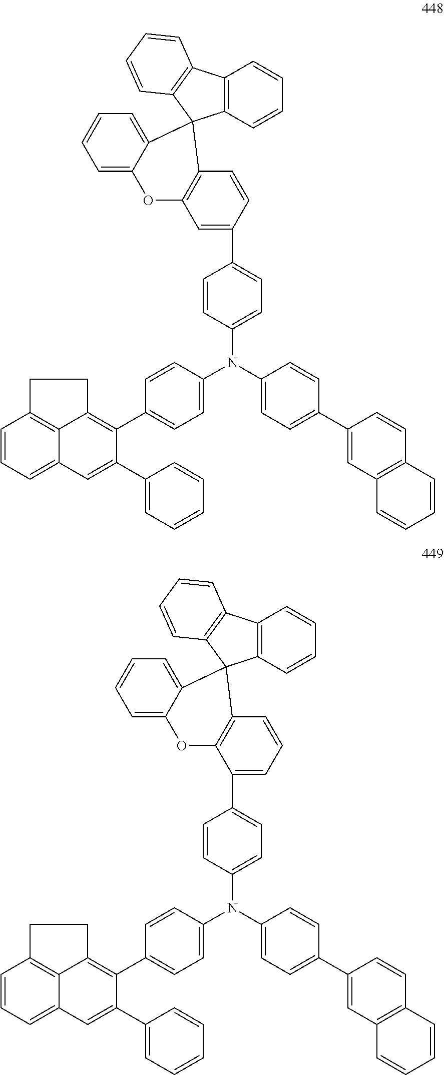 [SMILES: c1ccc(-c2cc3cccc4c3c(c2-c2ccc(N(c3ccc(-c5ccc6c(c5)Oc5ccccc5C65c6ccccc6-c6ccccc65)cc3)c3ccc(-c5ccc6ccccc6c5)cc3)cc2)CC4)cc1 ; c1ccc(-c2cc3cccc4c3c(c2-c2ccc(N(c3ccc(-c5ccc6ccccc6c5)cc3)c3ccc(-c5cccc6c5Oc5ccccc5C65c6ccccc6-c6ccccc65)cc3)cc2)CC4)cc1]

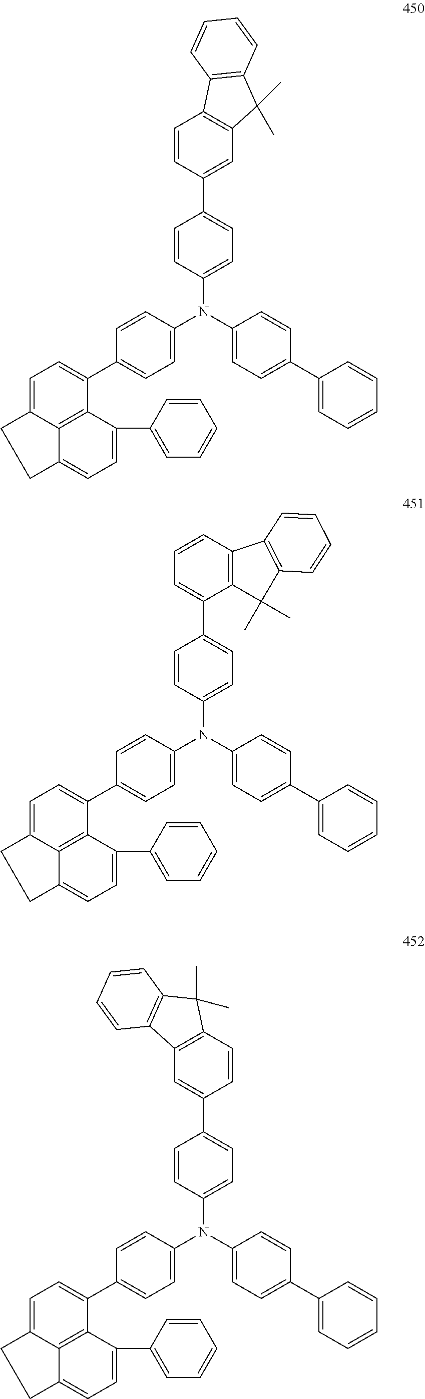 [SMILES: CC1(C)c2ccccc2-c2ccc(-c3ccc(N(c4ccc(-c5ccccc5)cc4)c4ccc(-c5ccc6c7c(ccc(-c8ccccc8)c57)CC6)cc4)cc3)cc21 ; CC1(C)c2ccccc2-c2cccc(-c3ccc(N(c4ccc(-c5ccccc5)cc4)c4ccc(-c5ccc6c7c(ccc(-c8ccccc8)c57)CC6)cc4)cc3)c21 ; CC1(C)c2ccccc2-c2cc(-c3ccc(N(c4ccc(-c5ccccc5)cc4)c4ccc(-c5ccc6c7c(ccc(-c8ccccc8)c57)CC6)cc4)cc3)ccc21]

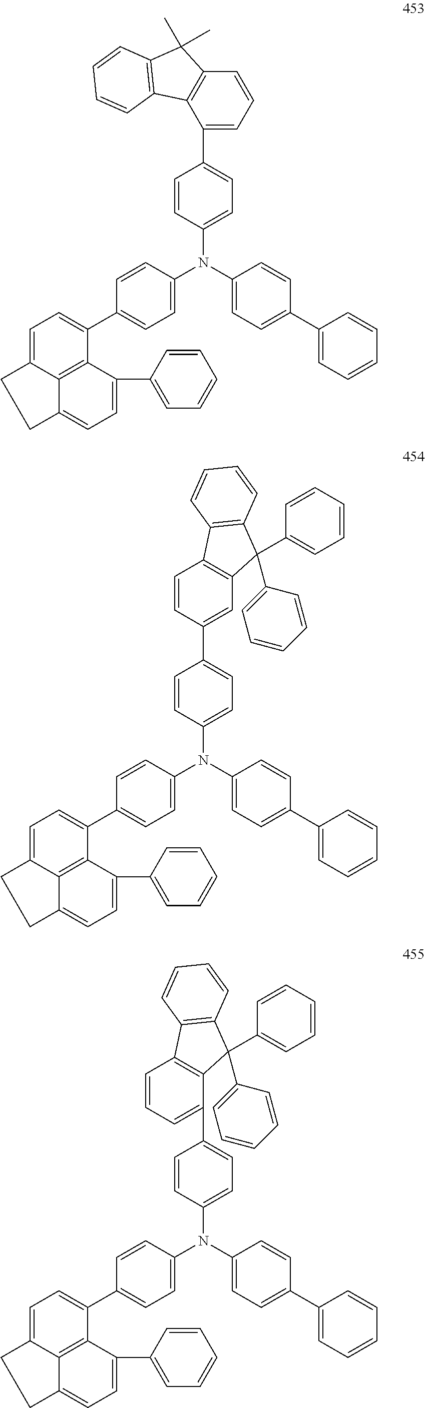 [SMILES: CC1(C)c2ccccc2-c2c(-c3ccc(N(c4ccc(-c5ccccc5)cc4)c4ccc(-c5ccc6c7c(ccc(-c8ccccc8)c57)CC6)cc4)cc3)cccc21 ; c1ccc(-c2ccc(N(c3ccc(-c4ccc5c(c4)C(c4ccccc4)(c4ccccc4)c4ccccc4-5)cc3)c3ccc(-c4ccc5c6c(ccc(-c7ccccc7)c46)CC5)cc3)cc2)cc1 ; c1ccc(-c2ccc(N(c3ccc(-c4cccc5c4C(c4ccccc4)(c4ccccc4)c4ccccc4-5)cc3)c3ccc(-c4ccc5c6c(ccc(-c7ccccc7)c46)CC5)cc3)cc2)cc1]

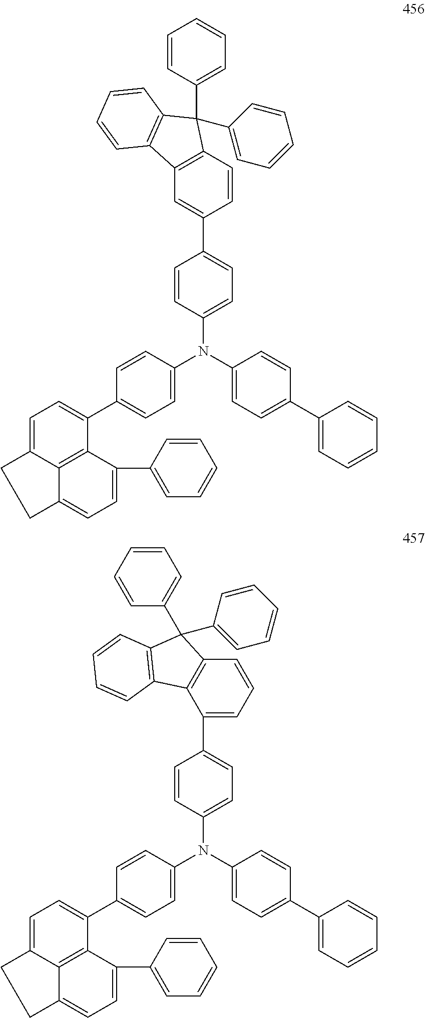 [SMILES: c1ccc(-c2ccc(N(c3ccc(-c4ccc5c(c4)-c4ccccc4C5(c4ccccc4)c4ccccc4)cc3)c3ccc(-c4ccc5c6c(ccc(-c7ccccc7)c46)CC5)cc3)cc2)cc1 ; c1ccc(-c2ccc(N(c3ccc(-c4cccc5c4-c4ccccc4C5(c4ccccc4)c4ccccc4)cc3)c3ccc(-c4ccc5c6c(ccc(-c7ccccc7)c46)CC5)cc3)cc2)cc1]

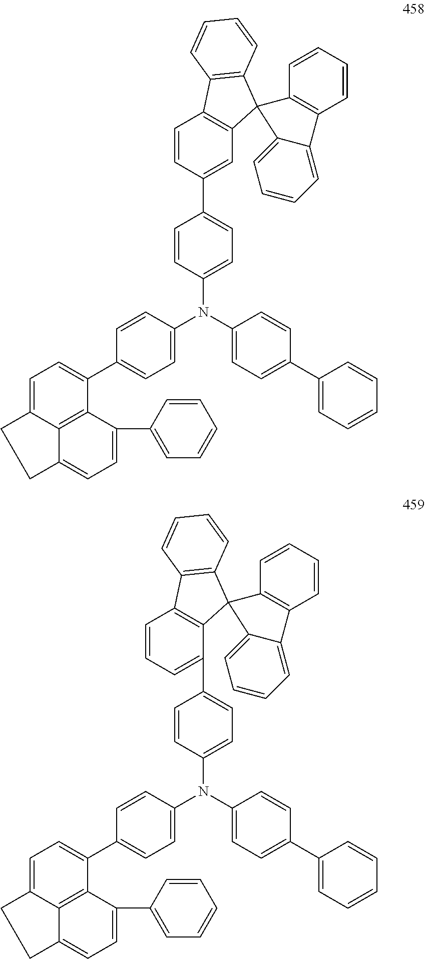 [SMILES: c1ccc(-c2ccc(N(c3ccc(-c4ccc5c(c4)C4(c6ccccc6-c6ccccc64)c4ccccc4-5)cc3)c3ccc(-c4ccc5c6c(ccc(-c7ccccc7)c46)CC5)cc3)cc2)cc1 ; c1ccc(-c2ccc(N(c3ccc(-c4cccc5c4C4(c6ccccc6-c6ccccc64)c4ccccc4-5)cc3)c3ccc(-c4ccc5c6c(ccc(-c7ccccc7)c46)CC5)cc3)cc2)cc1]

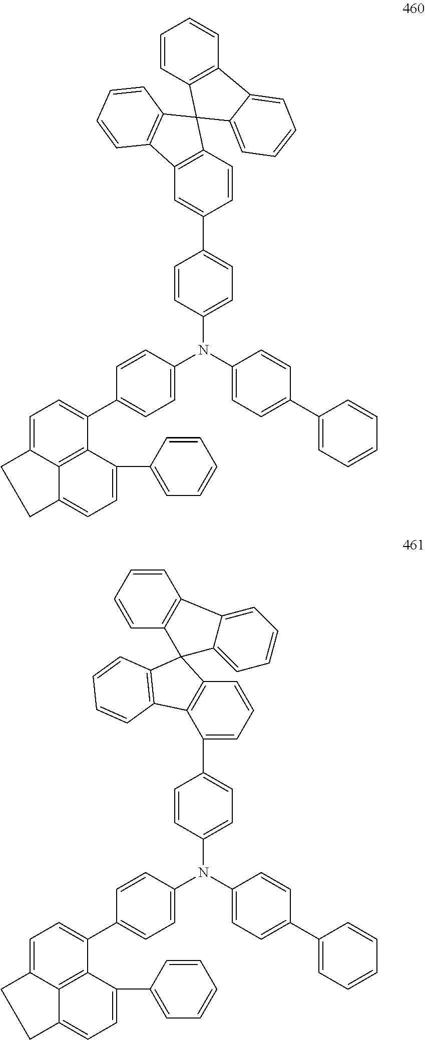 [SMILES: c1ccc(-c2ccc(N(c3ccc(-c4ccc5c(c4)-c4ccccc4C54c5ccccc5-c5ccccc54)cc3)c3ccc(-c4ccc5c6c(ccc(-c7ccccc7)c46)CC5)cc3)cc2)cc1 ; c1ccc(-c2ccc(N(c3ccc(-c4cccc5c4-c4ccccc4C54c5ccccc5-c5ccccc54)cc3)c3ccc(-c4ccc5c6c(ccc(-c7ccccc7)c46)CC5)cc3)cc2)cc1]

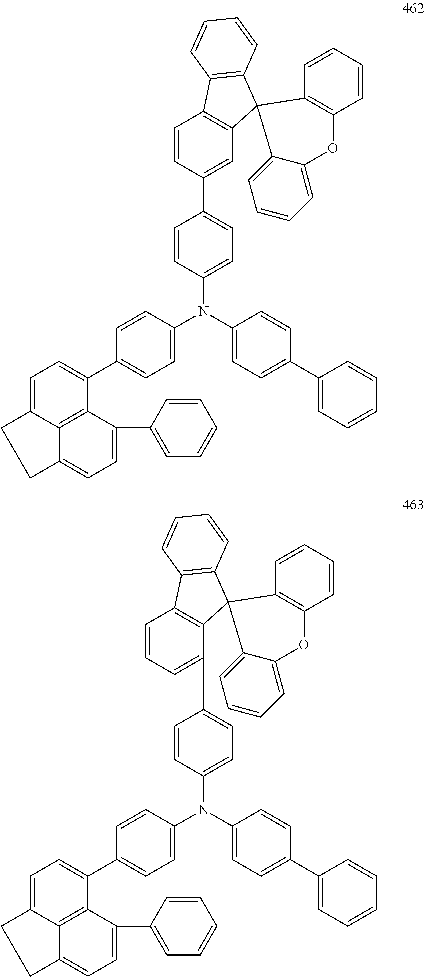 [SMILES: c1ccc(-c2ccc(N(c3ccc(-c4ccc5c(c4)C4(c6ccccc6Oc6ccccc64)c4ccccc4-5)cc3)c3ccc(-c4ccc5c6c(ccc(-c7ccccc7)c46)CC5)cc3)cc2)cc1 ; c1ccc(-c2ccc(N(c3ccc(-c4cccc5c4C4(c6ccccc6Oc6ccccc64)c4ccccc4-5)cc3)c3ccc(-c4ccc5c6c(ccc(-c7ccccc7)c46)CC5)cc3)cc2)cc1]

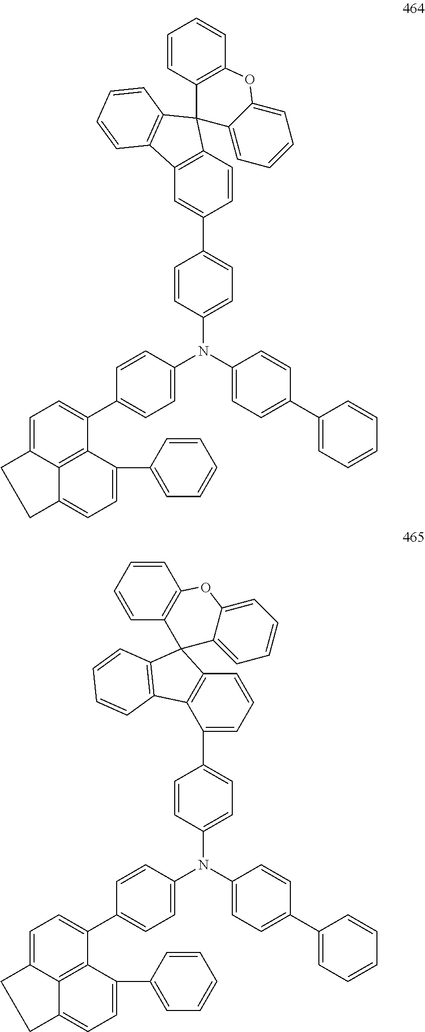 [SMILES: c1ccc(-c2ccc(N(c3ccc(-c4ccc5c(c4)-c4ccccc4C54c5ccccc5Oc5ccccc54)cc3)c3ccc(-c4ccc5c6c(ccc(-c7ccccc7)c46)CC5)cc3)cc2)cc1 ; c1ccc(-c2ccc(N(c3ccc(-c4cccc5c4-c4ccccc4C54c5ccccc5Oc5ccccc54)cc3)c3ccc(-c4ccc5c6c(ccc(-c7ccccc7)c46)CC5)cc3)cc2)cc1]

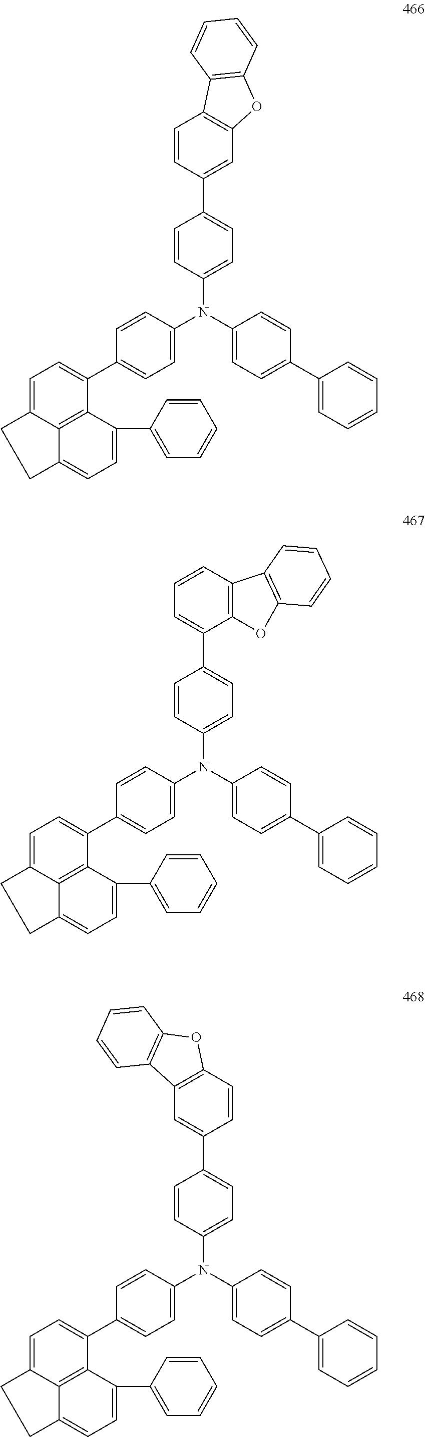 [SMILES: c1ccc(-c2ccc(N(c3ccc(-c4ccc5c(c4)oc4ccccc45)cc3)c3ccc(-c4ccc5c6c(ccc(-c7ccccc7)c46)CC5)cc3)cc2)cc1 ; c1ccc(-c2ccc(N(c3ccc(-c4cccc5c4oc4ccccc45)cc3)c3ccc(-c4ccc5c6c(ccc(-c7ccccc7)c46)CC5)cc3)cc2)cc1 ; c1ccc(-c2ccc(N(c3ccc(-c4ccc5oc6ccccc6c5c4)cc3)c3ccc(-c4ccc5c6c(ccc(-c7ccccc7)c46)CC5)cc3)cc2)cc1]

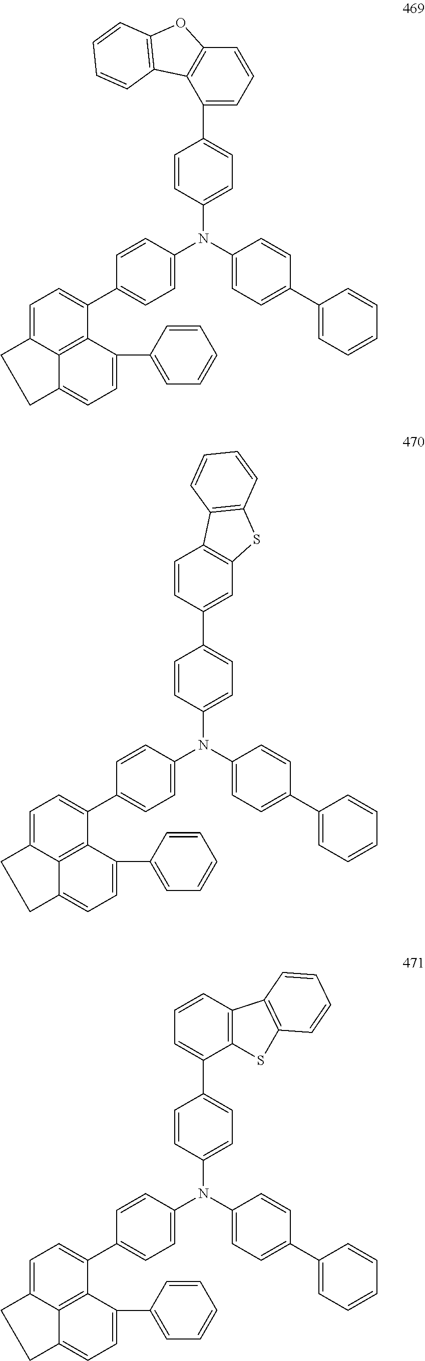 [SMILES: c1ccc(-c2ccc(N(c3ccc(-c4ccc5c6c(ccc(-c7ccccc7)c46)CC5)cc3)c3ccc(-c4cccc5oc6ccccc6c45)cc3)cc2)cc1 ; c1ccc(-c2ccc(N(c3ccc(-c4ccc5c(c4)sc4ccccc45)cc3)c3ccc(-c4ccc5c6c(ccc(-c7ccccc7)c46)CC5)cc3)cc2)cc1 ; c1ccc(-c2ccc(N(c3ccc(-c4cccc5c4sc4ccccc45)cc3)c3ccc(-c4ccc5c6c(ccc(-c7ccccc7)c46)CC5)cc3)cc2)cc1]

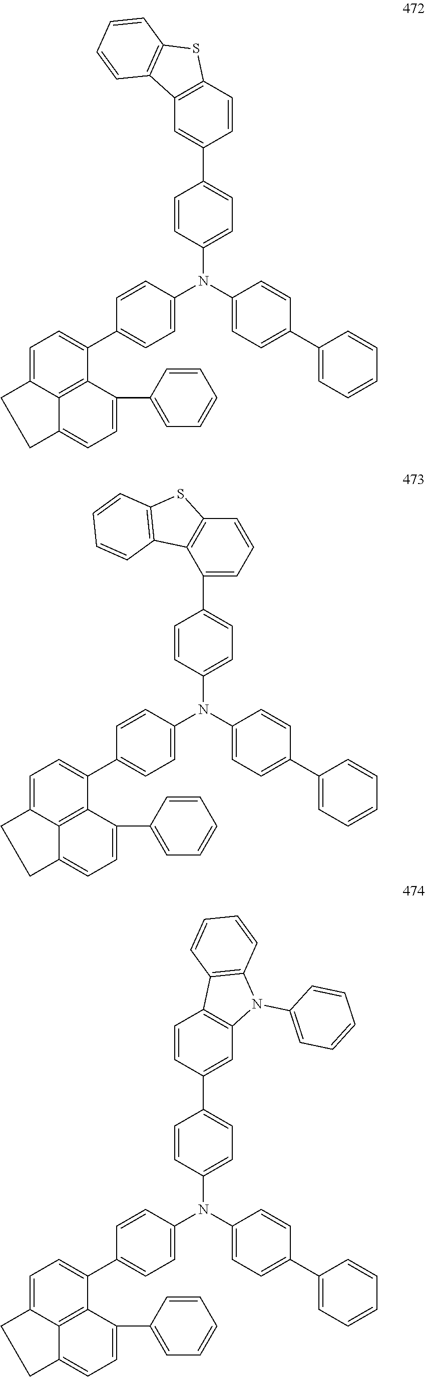 [SMILES: c1ccc(-c2ccc(N(c3ccc(-c4ccc5sc6ccccc6c5c4)cc3)c3ccc(-c4ccc5c6c(ccc(-c7ccccc7)c46)CC5)cc3)cc2)cc1 ; c1ccc(-c2ccc(N(c3ccc(-c4ccc5c6c(ccc(-c7ccccc7)c46)CC5)cc3)c3ccc(-c4cccc5sc6ccccc6c45)cc3)cc2)cc1 ; c1ccc(-c2ccc(N(c3ccc(-c4ccc5c6ccccc6n(-c6ccccc6)c5c4)cc3)c3ccc(-c4ccc5c6c(ccc(-c7ccccc7)c46)CC5)cc3)cc2)cc1]

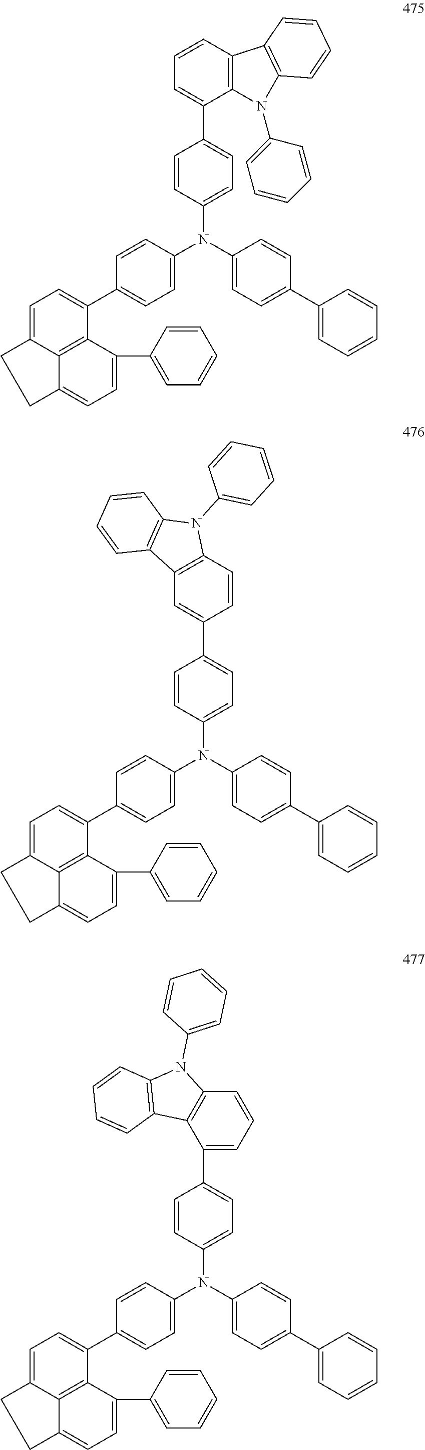 [SMILES: c1ccc(-c2ccc(N(c3ccc(-c4ccc5c6c(ccc(-c7ccccc7)c46)CC5)cc3)c3ccc(-c4cccc5c6ccccc6n(-c6ccccc6)c45)cc3)cc2)cc1 ; c1ccc(-c2ccc(N(c3ccc(-c4ccc5c(c4)c4ccccc4n5-c4ccccc4)cc3)c3ccc(-c4ccc5c6c(ccc(-c7ccccc7)c46)CC5)cc3)cc2)cc1 ; c1ccc(-c2ccc(N(c3ccc(-c4ccc5c6c(ccc(-c7ccccc7)c46)CC5)cc3)c3ccc(-c4cccc5c4c4ccccc4n5-c4ccccc4)cc3)cc2)cc1]

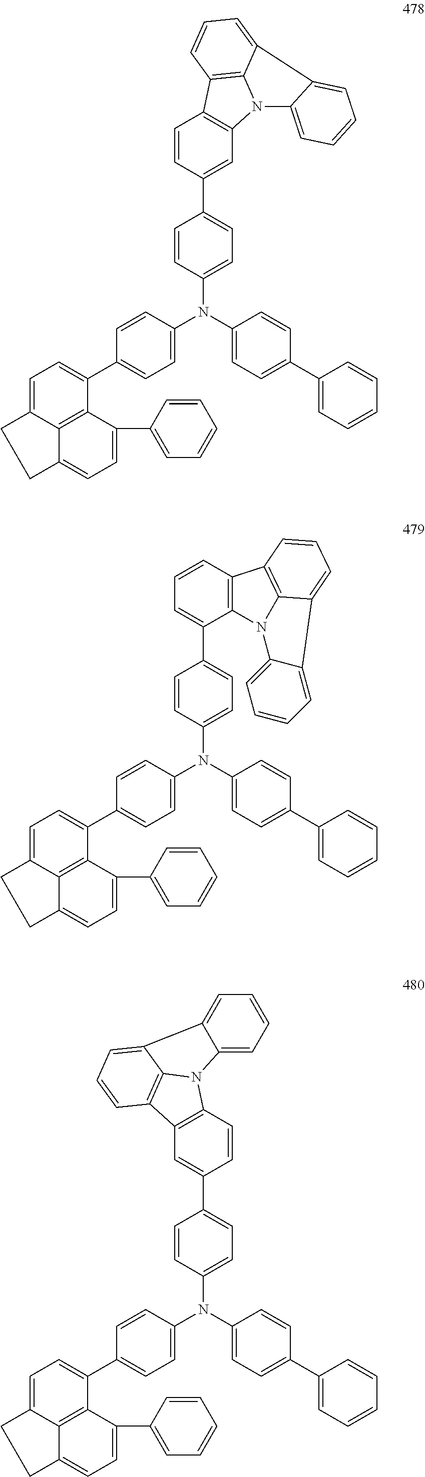 [SMILES: c1ccc(-c2ccc(N(c3ccc(-c4ccc5c6cccc7c8ccccc8n(c5c4)c76)cc3)c3ccc(-c4ccc5c6c(ccc(-c7ccccc7)c46)CC5)cc3)cc2)cc1 ; c1ccc(-c2ccc(N(c3ccc(-c4ccc5c6c(ccc(-c7ccccc7)c46)CC5)cc3)c3ccc(-c4cccc5c6cccc7c8ccccc8n(c45)c76)cc3)cc2)cc1 ; c1ccc(-c2ccc(N(c3ccc(-c4ccc5c(c4)c4cccc6c7ccccc7n5c64)cc3)c3ccc(-c4ccc5c6c(ccc(-c7ccccc7)c46)CC5)cc3)cc2)cc1]

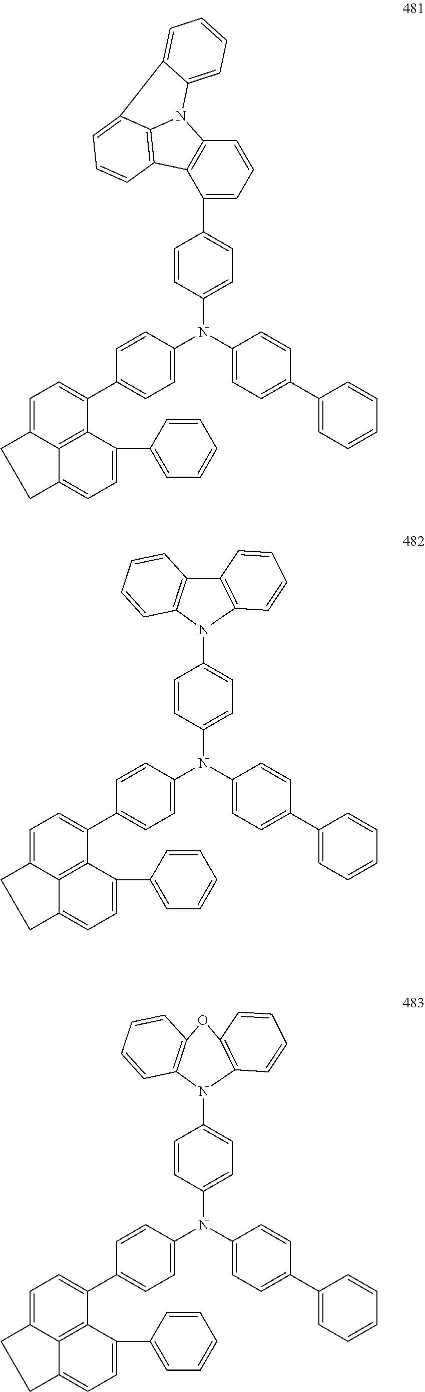 [SMILES: c1ccc(-c2ccc(N(c3ccc(-c4ccc5c6c(ccc(-c7ccccc7)c46)CC5)cc3)c3ccc(-c4cccc5c4c4cccc6c7ccccc7n5c64)cc3)cc2)cc1 ; c1ccc(-c2ccc(N(c3ccc(-c4ccc5c6c(ccc(-c7ccccc7)c46)CC5)cc3)c3ccc(-n4c5ccccc5c5ccccc54)cc3)cc2)cc1 ; c1ccc(-c2ccc(N(c3ccc(-c4ccc5c6c(ccc(-c7ccccc7)c46)CC5)cc3)c3ccc(N4c5ccccc5Oc5ccccc54)cc3)cc2)cc1]

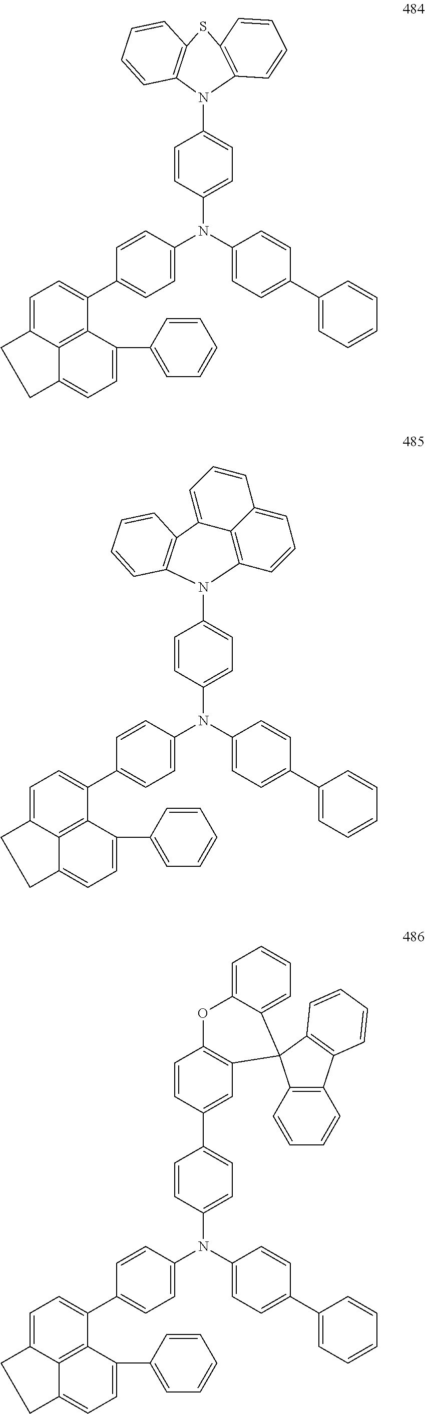 [SMILES: c1ccc(-c2ccc(N(c3ccc(-c4ccc5c6c(ccc(-c7ccccc7)c46)CC5)cc3)c3ccc(N4c5ccccc5Sc5ccccc54)cc3)cc2)cc1 ; c1ccc(-c2ccc(N(c3ccc(-c4ccc5c6c(ccc(-c7ccccc7)c46)CC5)cc3)c3ccc(N4c5ccccc5-c5cccc6cccc4c56)cc3)cc2)cc1 ; c1ccc(-c2ccc(N(c3ccc(-c4ccc5c(c4)C4(c6ccccc6O5)c5ccccc5-c5ccccc54)cc3)c3ccc(-c4ccc5c6c(ccc(-c7ccccc7)c46)CC5)cc3)cc2)cc1]

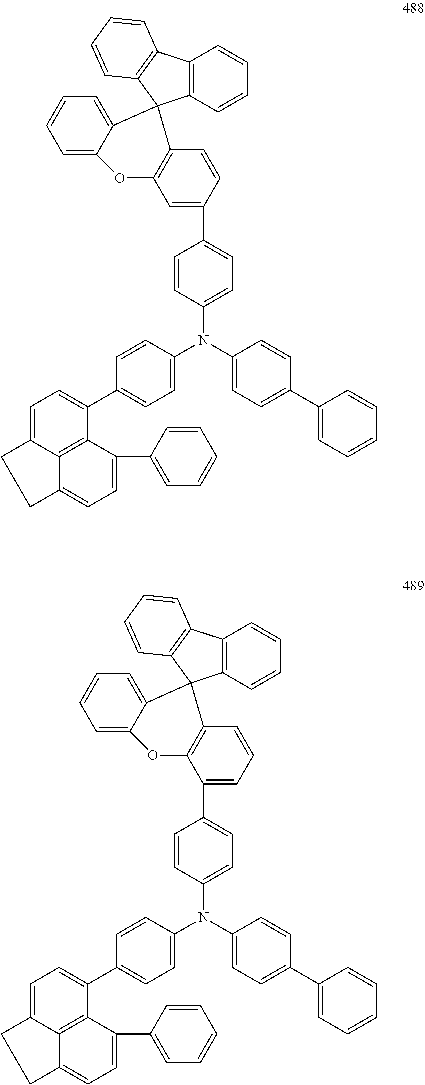 [SMILES: c1ccc(-c2ccc(N(c3ccc(-c4ccc5c(c4)Oc4ccccc4C54c5ccccc5-c5ccccc54)cc3)c3ccc(-c4ccc5c6c(ccc(-c7ccccc7)c46)CC5)cc3)cc2)cc1 ; c1ccc(-c2ccc(N(c3ccc(-c4cccc5c4Oc4ccccc4C54c5ccccc5-c5ccccc54)cc3)c3ccc(-c4ccc5c6c(ccc(-c7ccccc7)c46)CC5)cc3)cc2)cc1]

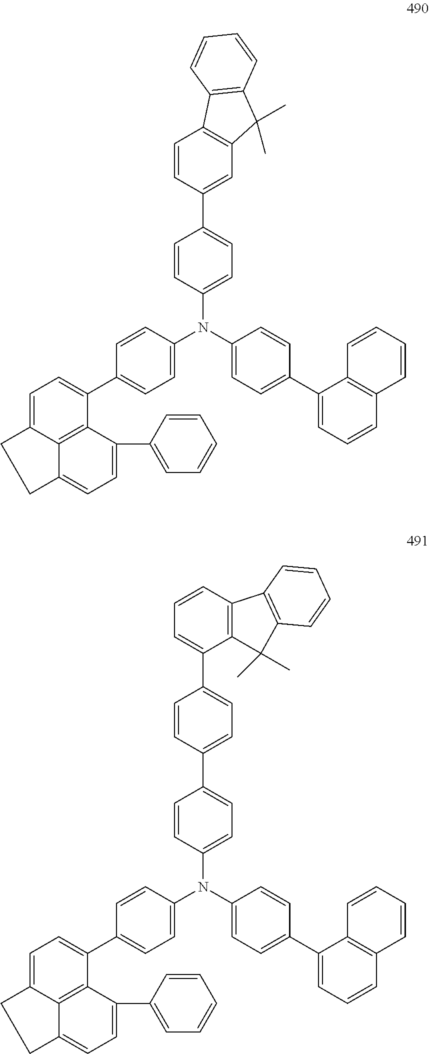 [SMILES: CC1(C)c2ccccc2-c2ccc(-c3ccc(N(c4ccc(-c5cccc6ccccc56)cc4)c4ccc(-c5ccc6c7c(ccc(-c8ccccc8)c57)CC6)cc4)cc3)cc21 ; CC1(C)c2ccccc2-c2cccc(-c3ccc(-c4ccc(N(c5ccc(-c6cccc7ccccc67)cc5)c5ccc(-c6ccc7c8c(ccc(-c9ccccc9)c68)CC7)cc5)cc4)cc3)c21]

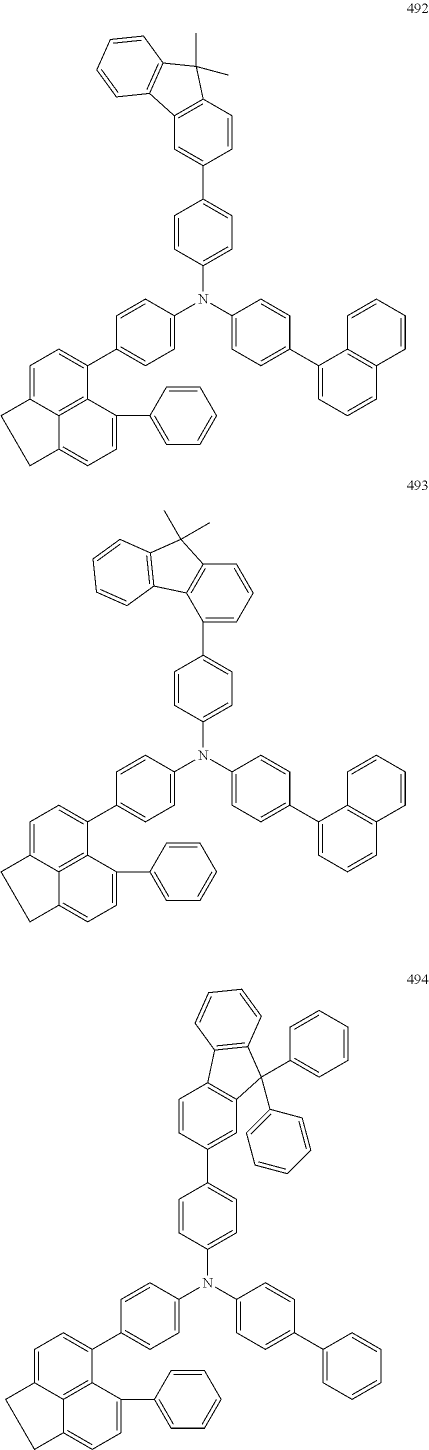 [SMILES: CC1(C)c2ccccc2-c2cc(-c3ccc(N(c4ccc(-c5cccc6ccccc56)cc4)c4ccc(-c5ccc6c7c(ccc(-c8ccccc8)c57)CC6)cc4)cc3)ccc21 ; CC1(C)c2ccccc2-c2c(-c3ccc(N(c4ccc(-c5cccc6ccccc56)cc4)c4ccc(-c5ccc6c7c(ccc(-c8ccccc8)c57)CC6)cc4)cc3)cccc21 ; c1ccc(-c2ccc(N(c3ccc(-c4ccc5c(c4)C(c4ccccc4)(c4ccccc4)c4ccccc4-5)cc3)c3ccc(-c4ccc5c6c(ccc(-c7ccccc7)c46)CC5)cc3)cc2)cc1]

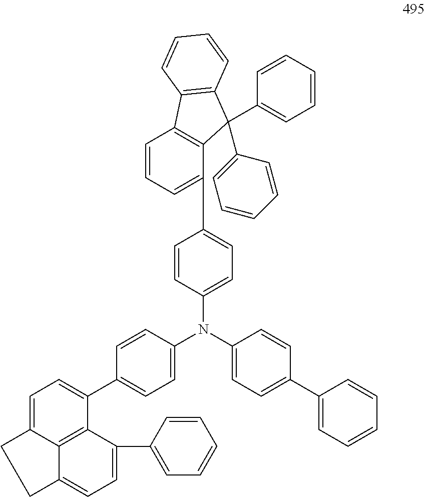 [SMILES: c1ccc(-c2ccc(N(c3ccc(-c4cccc5c4C(c4ccccc4)(c4ccccc4)c4ccccc4-5)cc3)c3ccc(-c4ccc5c6c(ccc(-c7ccccc7)c46)CC5)cc3)cc2)cc1]

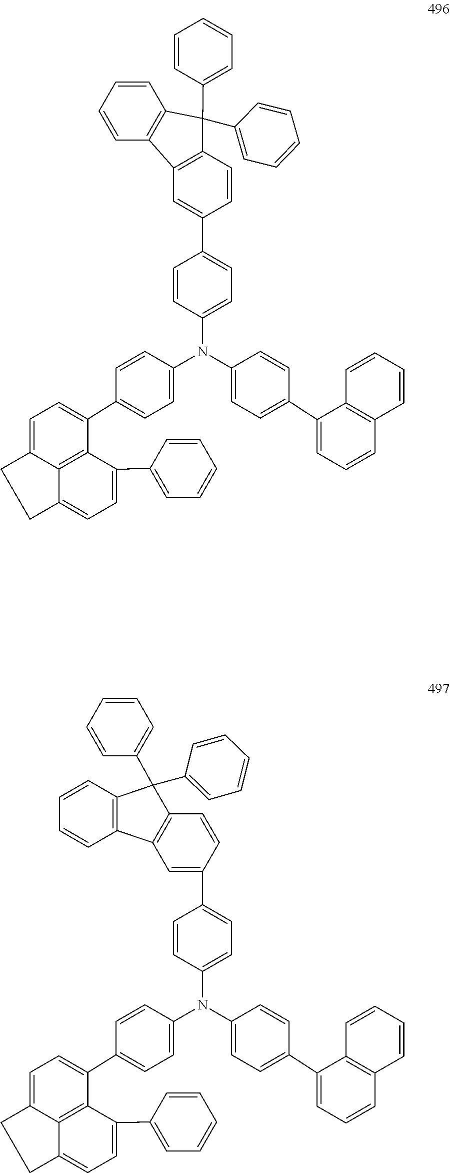 [SMILES: c1ccc(-c2ccc3c4c(ccc(-c5ccc(N(c6ccc(-c7ccc8c(c7)-c7ccccc7C8(c7ccccc7)c7ccccc7)cc6)c6ccc(-c7cccc8ccccc78)cc6)cc5)c24)CC3)cc1 ; c1ccc(-c2ccc3c4c(ccc(-c5ccc(N(c6ccc(-c7ccc8c(c7)-c7ccccc7C8(c7ccccc7)c7ccccc7)cc6)c6ccc(-c7cccc8ccccc78)cc6)cc5)c24)CC3)cc1]

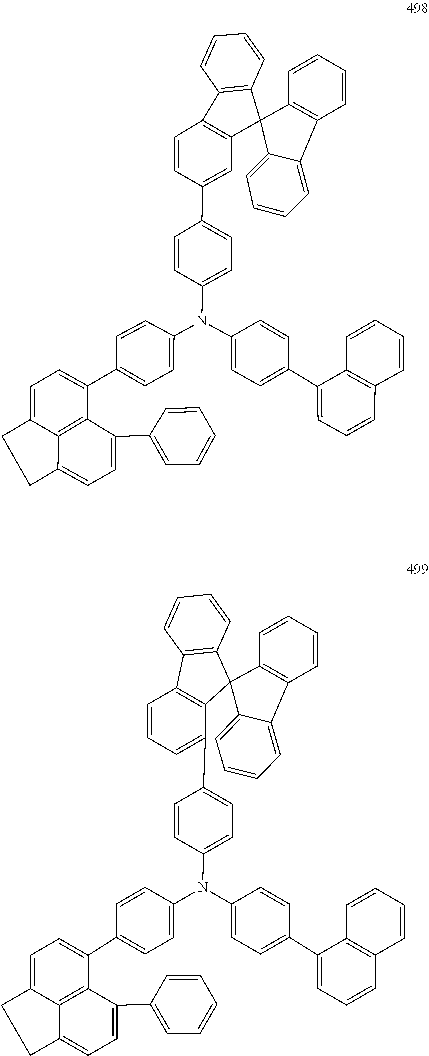 [SMILES: c1ccc(-c2ccc3c4c(ccc(-c5ccc(N(c6ccc(-c7ccc8c(c7)C7(c9ccccc9-c9ccccc97)c7ccccc7-8)cc6)c6ccc(-c7cccc8ccccc78)cc6)cc5)c24)CC3)cc1 ; c1ccc(-c2ccc3c4c(ccc(-c5ccc(N(c6ccc(-c7cccc8c7C7(c9ccccc9-c9ccccc97)c7ccccc7-8)cc6)c6ccc(-c7cccc8ccccc78)cc6)cc5)c24)CC3)cc1]

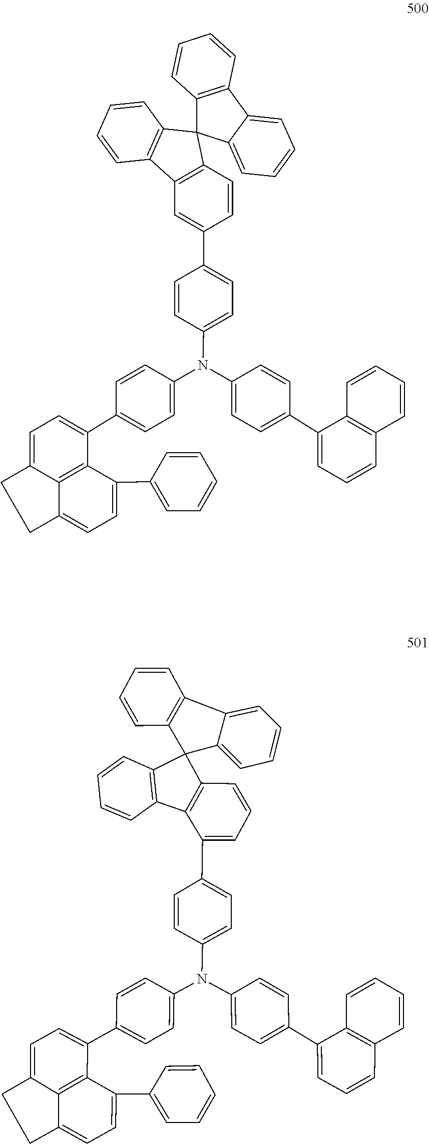 [SMILES: c1ccc(-c2ccc3c4c(ccc(-c5ccc(N(c6ccc(-c7ccc8c(c7)-c7ccccc7C87c8ccccc8-c8ccccc87)cc6)c6ccc(-c7cccc8ccccc78)cc6)cc5)c24)CC3)cc1 ; c1ccc(-c2ccc3c4c(ccc(-c5ccc(N(c6ccc(-c7cccc8c7-c7ccccc7C87c8ccccc8-c8ccccc87)cc6)c6ccc(-c7cccc8ccccc78)cc6)cc5)c24)CC3)cc1]

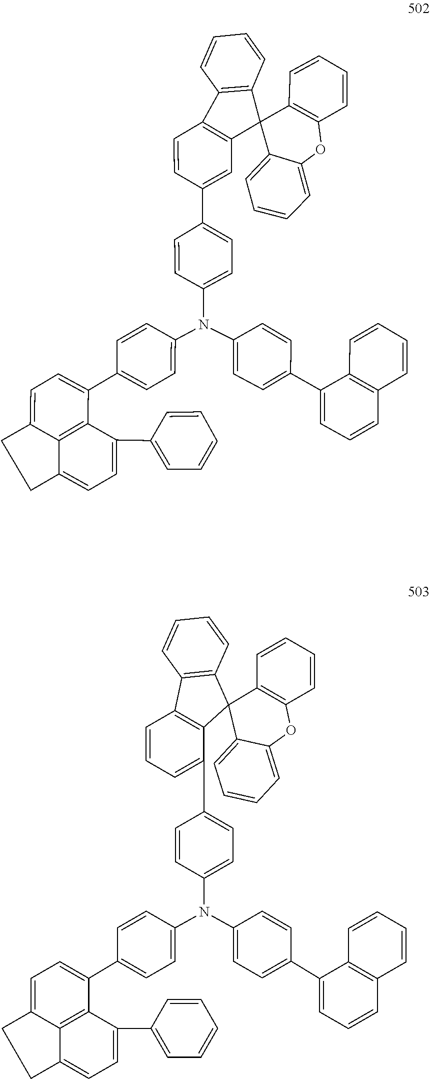 [SMILES: c1ccc(-c2ccc3c4c(ccc(-c5ccc(N(c6ccc(-c7ccc8c(c7)C7(c9ccccc9Oc9ccccc97)c7ccccc7-8)cc6)c6ccc(-c7cccc8ccccc78)cc6)cc5)c24)CC3)cc1 ; c1ccc(-c2ccc3c4c(ccc(-c5ccc(N(c6ccc(-c7cccc8c7C7(c9ccccc9Oc9ccccc97)c7ccccc7-8)cc6)c6ccc(-c7cccc8ccccc78)cc6)cc5)c24)CC3)cc1]

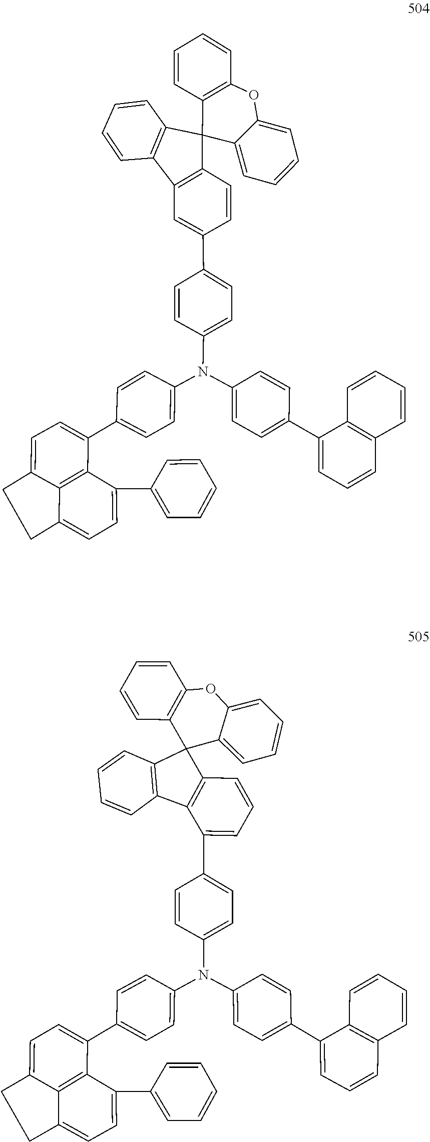 [SMILES: c1ccc(-c2ccc3c4c(ccc(-c5ccc(N(c6ccc(-c7ccc8c(c7)-c7ccccc7C87c8ccccc8Oc8ccccc87)cc6)c6ccc(-c7cccc8ccccc78)cc6)cc5)c24)CC3)cc1 ; c1ccc(-c2ccc3c4c(ccc(-c5ccc(N(c6ccc(-c7cccc8c7-c7ccccc7C87c8ccccc8Oc8ccccc87)cc6)c6ccc(-c7cccc8ccccc78)cc6)cc5)c24)CC3)cc1]

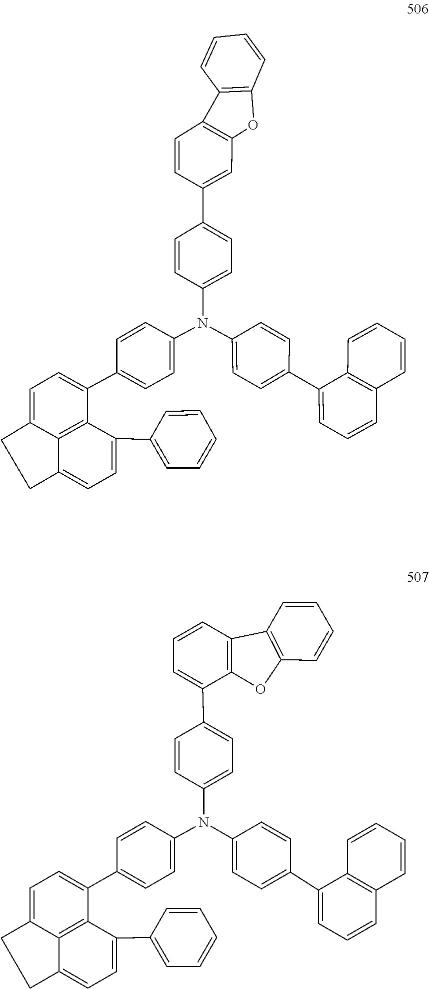 [SMILES: c1ccc(-c2ccc3c4c(ccc(-c5ccc(N(c6ccc(-c7ccc8c(c7)oc7ccccc78)cc6)c6ccc(-c7cccc8ccccc78)cc6)cc5)c24)CC3)cc1 ; c1ccc(-c2ccc3c4c(ccc(-c5ccc(N(c6ccc(-c7cccc8ccccc78)cc6)c6ccc(-c7cccc8c7oc7ccccc78)cc6)cc5)c24)CC3)cc1]

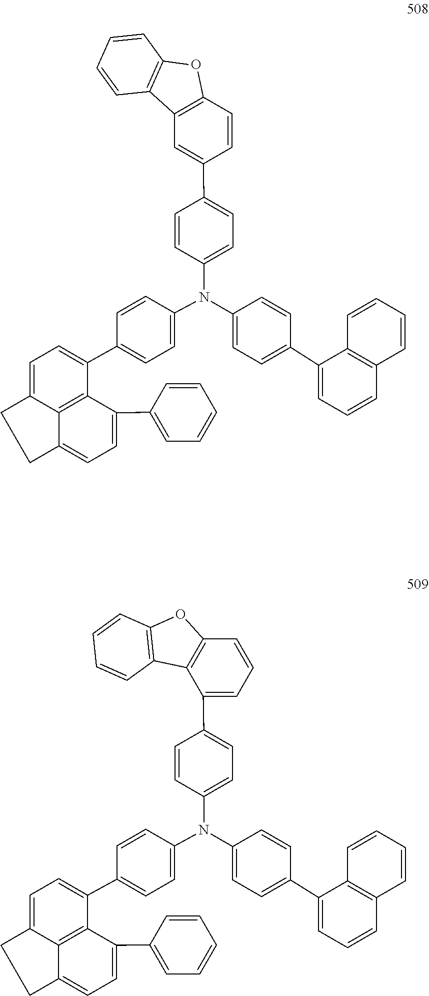 [SMILES: c1ccc(-c2ccc3c4c(ccc(-c5ccc(N(c6ccc(-c7ccc8oc9ccccc9c8c7)cc6)c6ccc(-c7cccc8ccccc78)cc6)cc5)c24)CC3)cc1 ; c1ccc(-c2ccc3c4c(ccc(-c5ccc(N(c6ccc(-c7cccc8ccccc78)cc6)c6ccc(-c7cccc8oc9ccccc9c78)cc6)cc5)c24)CC3)cc1]

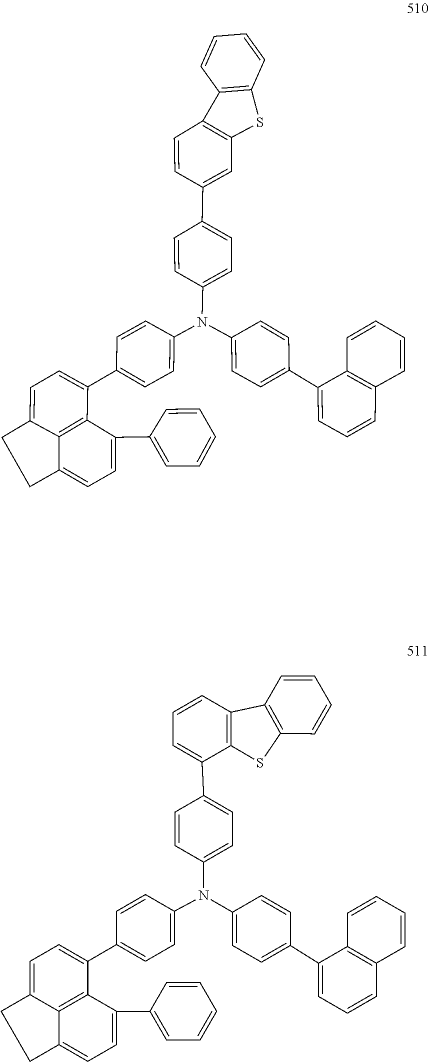 [SMILES: c1ccc(-c2ccc3c4c(ccc(-c5ccc(N(c6ccc(-c7ccc8c(c7)sc7ccccc78)cc6)c6ccc(-c7cccc8ccccc78)cc6)cc5)c24)CC3)cc1 ; c1ccc(-c2ccc3c4c(ccc(-c5ccc(N(c6ccc(-c7cccc8ccccc78)cc6)c6ccc(-c7cccc8c7sc7ccccc78)cc6)cc5)c24)CC3)cc1]

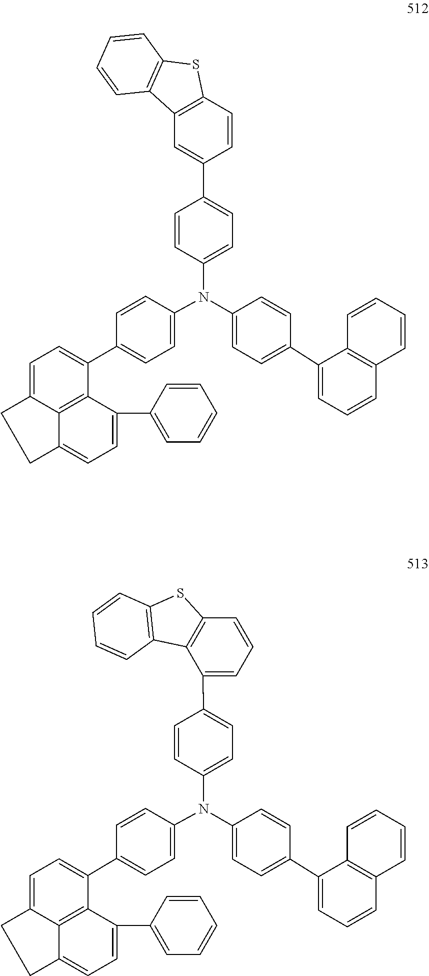 [SMILES: c1ccc(-c2ccc3c4c(ccc(-c5ccc(N(c6ccc(-c7ccc8sc9ccccc9c8c7)cc6)c6ccc(-c7cccc8ccccc78)cc6)cc5)c24)CC3)cc1 ; c1ccc(-c2ccc3c4c(ccc(-c5ccc(N(c6ccc(-c7cccc8ccccc78)cc6)c6ccc(-c7cccc8sc9ccccc9c78)cc6)cc5)c24)CC3)cc1]

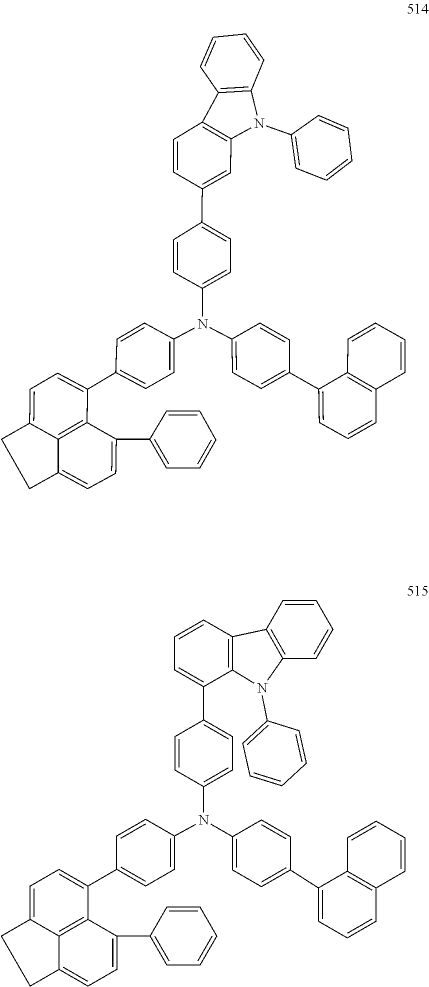 [SMILES: c1ccc(-c2ccc3c4c(ccc(-c5ccc(N(c6ccc(-c7ccc8c9ccccc9n(-c9ccccc9)c8c7)cc6)c6ccc(-c7cccc8ccccc78)cc6)cc5)c24)CC3)cc1 ; c1ccc(-c2ccc3c4c(ccc(-c5ccc(N(c6ccc(-c7cccc8ccccc78)cc6)c6ccc(-c7cccc8c9ccccc9n(-c9ccccc9)c78)cc6)cc5)c24)CC3)cc1]

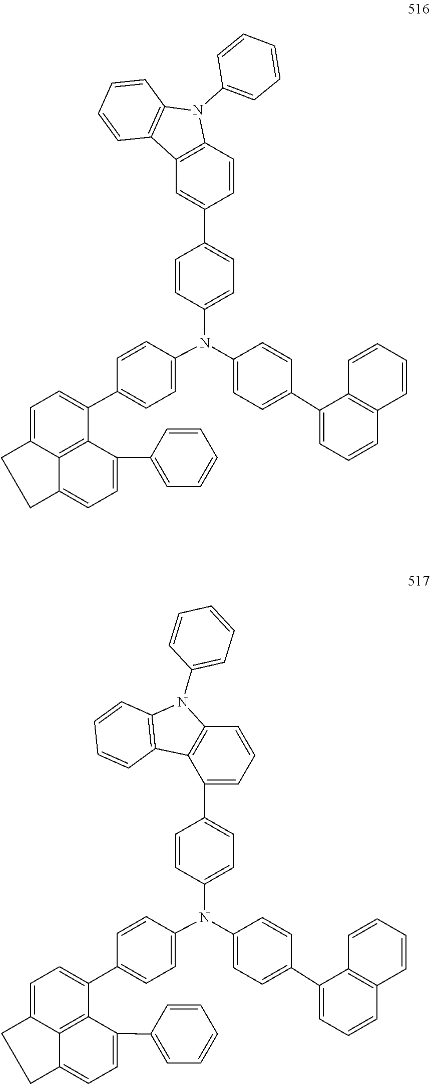 [SMILES: c1ccc(-c2ccc3c4c(ccc(-c5ccc(N(c6ccc(-c7ccc8c(c7)c7ccccc7n8-c7ccccc7)cc6)c6ccc(-c7cccc8ccccc78)cc6)cc5)c24)CC3)cc1 ; c1ccc(-c2ccc3c4c(ccc(-c5ccc(N(c6ccc(-c7cccc8ccccc78)cc6)c6ccc(-c7cccc8c7c7ccccc7n8-c7ccccc7)cc6)cc5)c24)CC3)cc1]

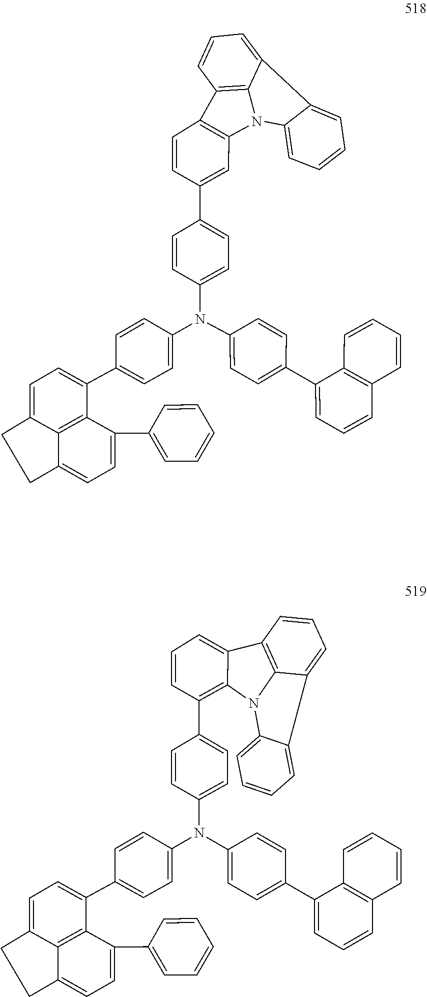 [SMILES: c1ccc(-c2ccc3c4c(ccc(-c5ccc(N(c6ccc(-c7ccc8c9cccc%10c%11ccccc%11n(c8c7)c%109)cc6)c6ccc(-c7cccc8ccccc78)cc6)cc5)c24)CC3)cc1 ; c1ccc(-c2ccc3c4c(ccc(-c5ccc(N(c6ccc(-c7cccc8ccccc78)cc6)c6ccc(-c7cccc8c9cccc%10c%11ccccc%11n(c78)c%109)cc6)cc5)c24)CC3)cc1]

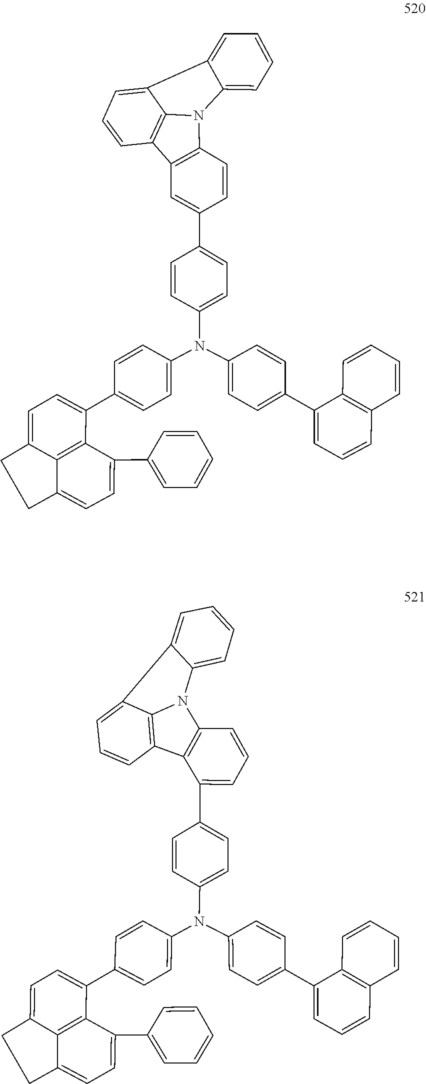 [SMILES: c1ccc(-c2ccc3c4c(ccc(-c5ccc(N(c6ccc(-c7ccc8c(c7)c7cccc9c%10ccccc%10n8c97)cc6)c6ccc(-c7cccc8ccccc78)cc6)cc5)c24)CC3)cc1 ; c1ccc(-c2ccc3c4c(ccc(-c5ccc(N(c6ccc(-c7cccc8ccccc78)cc6)c6ccc(-c7cccc8c7c7cccc9c%10ccccc%10n8c97)cc6)cc5)c24)CC3)cc1]

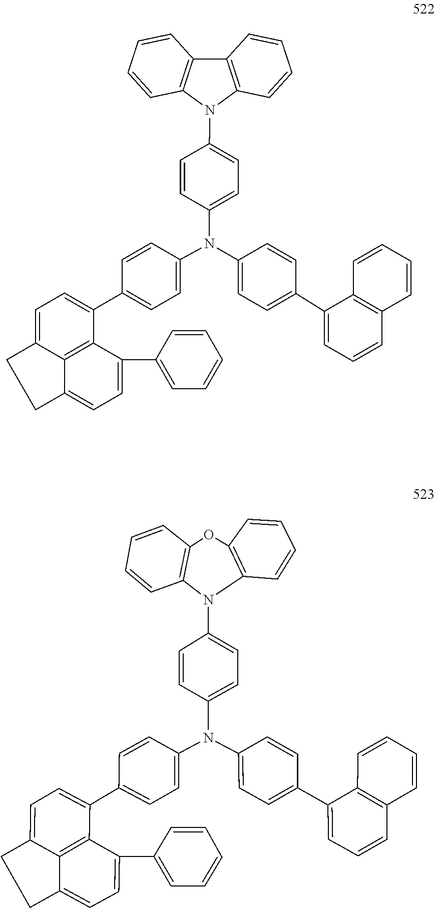 [SMILES: c1ccc(-c2ccc3c4c(ccc(-c5ccc(N(c6ccc(-c7cccc8ccccc78)cc6)c6ccc(-n7c8ccccc8c8ccccc87)cc6)cc5)c24)CC3)cc1 ; c1ccc(-c2ccc3c4c(ccc(-c5ccc(N(c6ccc(-c7cccc8ccccc78)cc6)c6ccc(N7c8ccccc8Oc8ccccc87)cc6)cc5)c24)CC3)cc1]

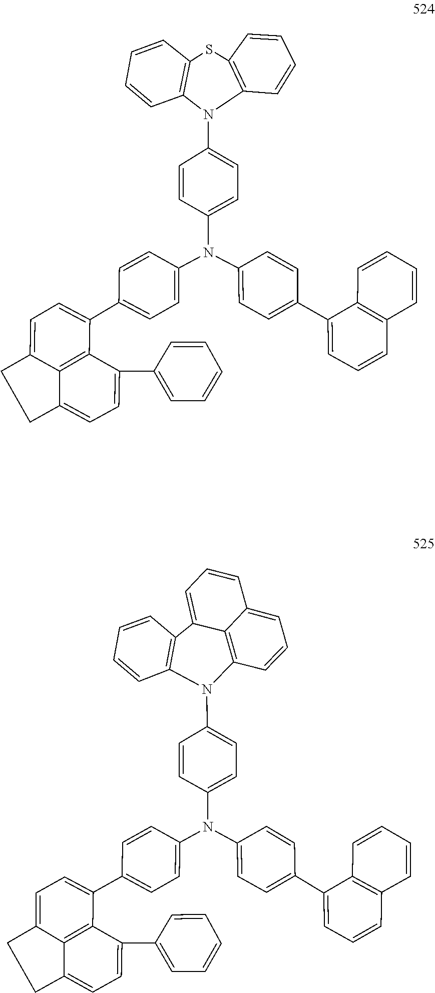 [SMILES: c1ccc(-c2ccc3c4c(ccc(-c5ccc(N(c6ccc(-c7cccc8ccccc78)cc6)c6ccc(N7c8ccccc8Sc8ccccc87)cc6)cc5)c24)CC3)cc1 ; c1ccc(-c2ccc3c4c(ccc(-c5ccc(N(c6ccc(-c7cccc8ccccc78)cc6)c6ccc(N7c8ccccc8-c8cccc9cccc7c89)cc6)cc5)c24)CC3)cc1]

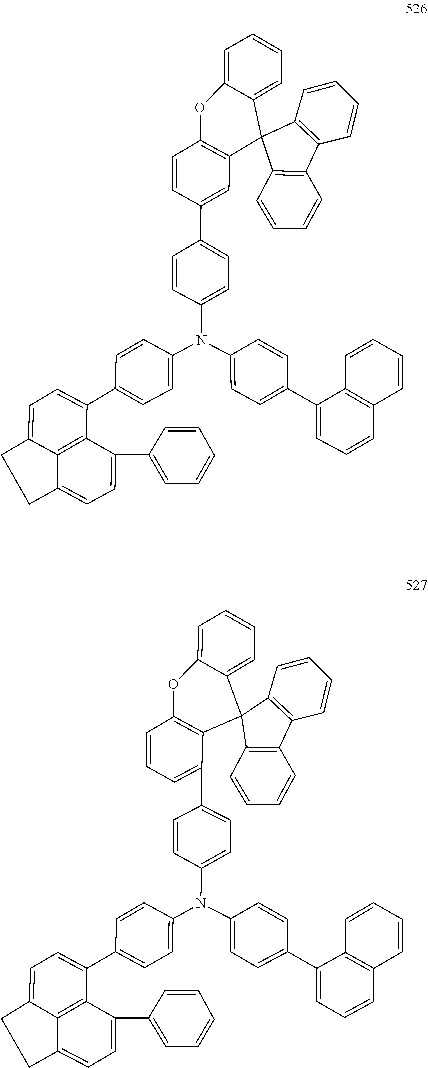 [SMILES: c1ccc(-c2ccc3c4c(ccc(-c5ccc(N(c6ccc(-c7ccc8c(c7)C7(c9ccccc9O8)c8ccccc8-c8ccccc87)cc6)c6ccc(-c7cccc8ccccc78)cc6)cc5)c24)CC3)cc1 ; c1ccc(-c2ccc3c4c(ccc(-c5ccc(N(c6ccc(-c7cccc8c7C7(c9ccccc9O8)c8ccccc8-c8ccccc87)cc6)c6ccc(-c7cccc8ccccc78)cc6)cc5)c24)CC3)cc1]

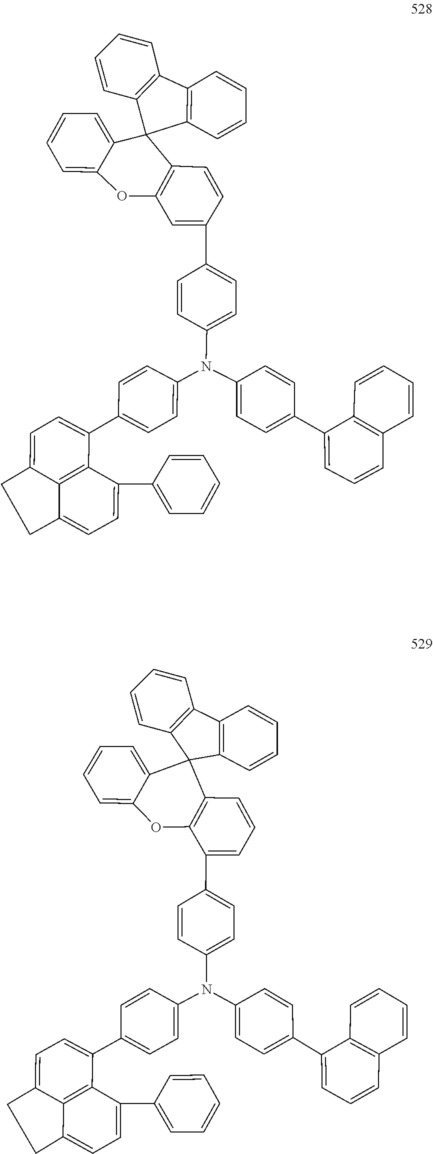 [SMILES: c1ccc(-c2ccc3c4c(ccc(-c5ccc(N(c6ccc(-c7ccc8c(c7)Oc7ccccc7C87c8ccccc8-c8ccccc87)cc6)c6ccc(-c7cccc8ccccc78)cc6)cc5)c24)CC3)cc1 ; c1ccc(-c2ccc3c4c(ccc(-c5ccc(N(c6ccc(-c7cccc8c7Oc7ccccc7C87c8ccccc8-c8ccccc87)cc6)c6ccc(-c7cccc8ccccc78)cc6)cc5)c24)CC3)cc1]

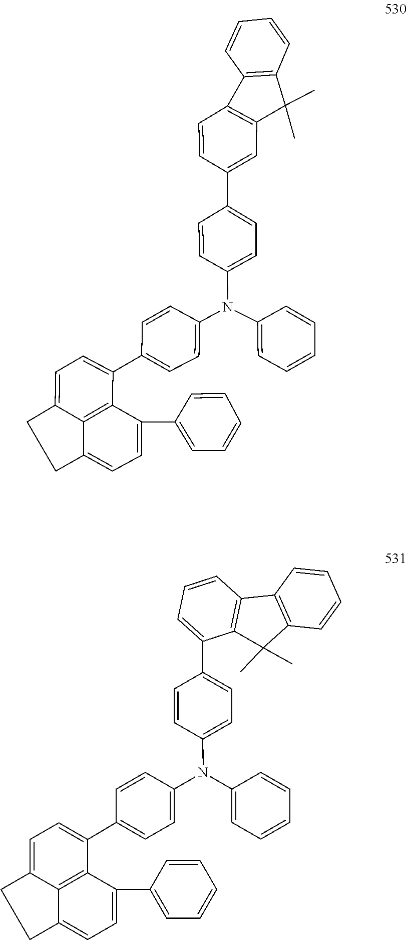 [SMILES: CC1(C)c2ccccc2-c2ccc(-c3ccc(N(c4ccccc4)c4ccc(-c5ccc6c7c(ccc(-c8ccccc8)c57)CC6)cc4)cc3)cc21 ; CC1(C)c2ccccc2-c2cccc(-c3ccc(N(c4ccccc4)c4ccc(-c5ccc6c7c(ccc(-c8ccccc8)c57)CC6)cc4)cc3)c21]

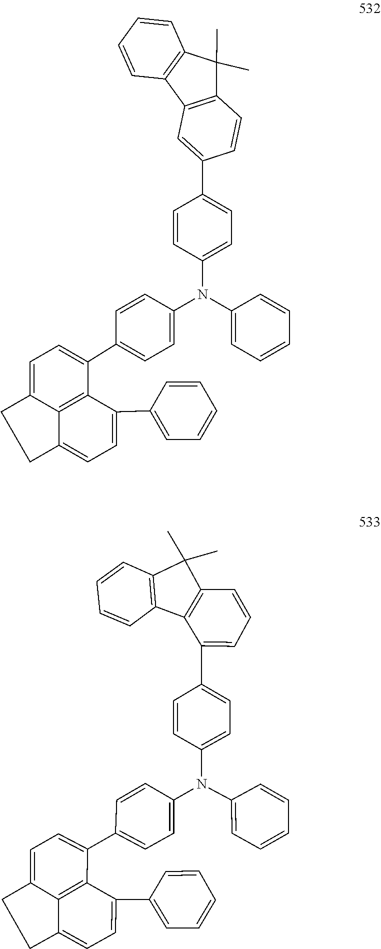 [SMILES: CC1(C)c2ccccc2-c2cc(-c3ccc(N(c4ccccc4)c4ccc(-c5ccc6c7c(ccc(-c8ccccc8)c57)CC6)cc4)cc3)ccc21 ; CC1(C)c2ccccc2-c2c(-c3ccc(N(c4ccccc4)c4ccc(-c5ccc6c7c(ccc(-c8ccccc8)c57)CC6)cc4)cc3)cccc21]

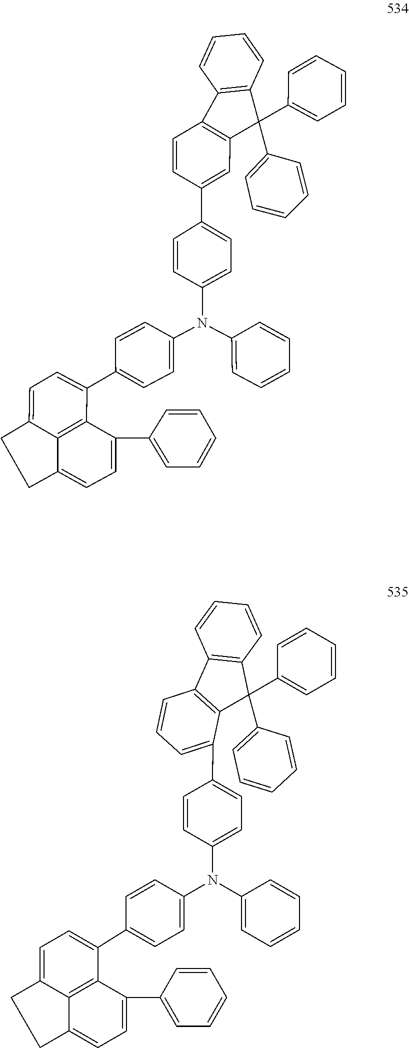 [SMILES: c1ccc(-c2ccc3c4c(ccc(-c5ccc(N(c6ccccc6)c6ccc(-c7ccc8c(c7)C(c7ccccc7)(c7ccccc7)c7ccccc7-8)cc6)cc5)c24)CC3)cc1 ; c1ccc(-c2ccc3c4c(ccc(-c5ccc(N(c6ccccc6)c6ccc(-c7cccc8c7C(c7ccccc7)(c7ccccc7)c7ccccc7-8)cc6)cc5)c24)CC3)cc1]

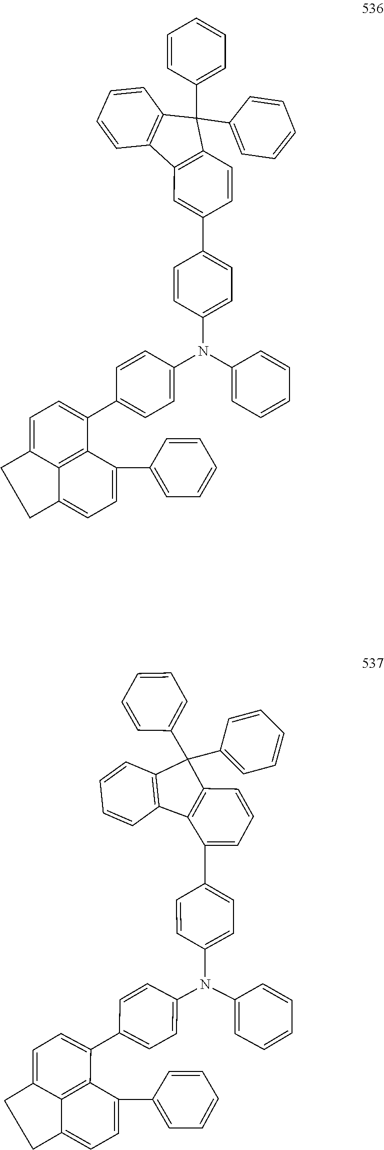 [SMILES: c1ccc(-c2ccc3c4c(ccc(-c5ccc(N(c6ccccc6)c6ccc(-c7ccc8c(c7)-c7ccccc7C8(c7ccccc7)c7ccccc7)cc6)cc5)c24)CC3)cc1 ; c1ccc(-c2ccc3c4c(ccc(-c5ccc(N(c6ccccc6)c6ccc(-c7cccc8c7-c7ccccc7C8(c7ccccc7)c7ccccc7)cc6)cc5)c24)CC3)cc1]

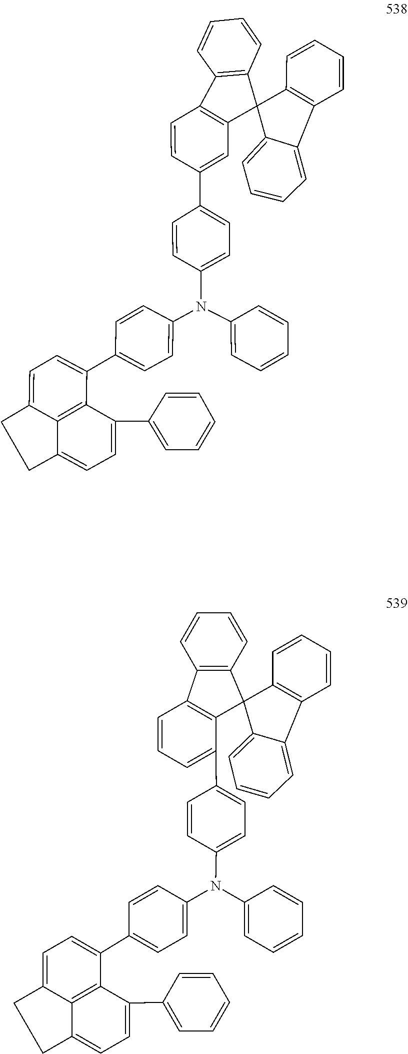 [SMILES: c1ccc(-c2ccc3c4c(ccc(-c5ccc(N(c6ccccc6)c6ccc(-c7ccc8c(c7)C7(c9ccccc9-c9ccccc97)c7ccccc7-8)cc6)cc5)c24)CC3)cc1 ; c1ccc(-c2ccc3c4c(ccc(-c5ccc(N(c6ccccc6)c6ccc(-c7cccc8c7C7(c9ccccc9-c9ccccc97)c7ccccc7-8)cc6)cc5)c24)CC3)cc1]

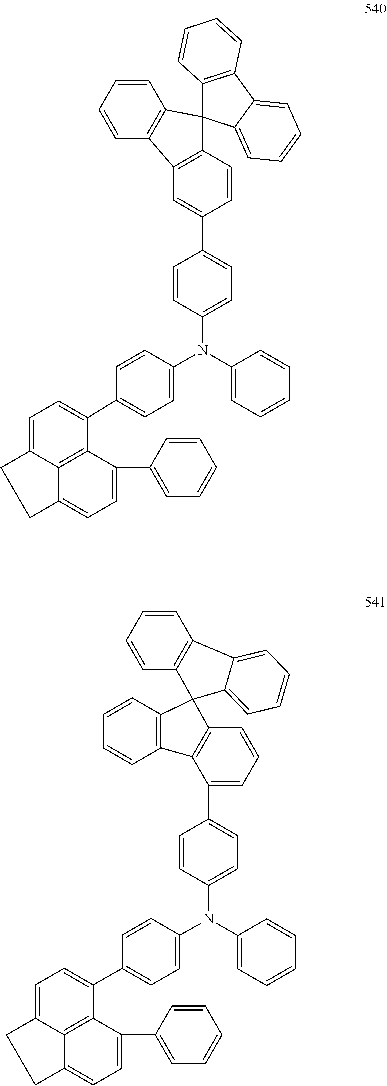 [SMILES: c1ccc(-c2ccc3c4c(ccc(-c5ccc(N(c6ccccc6)c6ccc(-c7ccc8c(c7)-c7ccccc7C87c8ccccc8-c8ccccc87)cc6)cc5)c24)CC3)cc1 ; c1ccc(-c2ccc3c4c(ccc(-c5ccc(N(c6ccccc6)c6ccc(-c7cccc8c7-c7ccccc7C87c8ccccc8-c8ccccc87)cc6)cc5)c24)CC3)cc1]

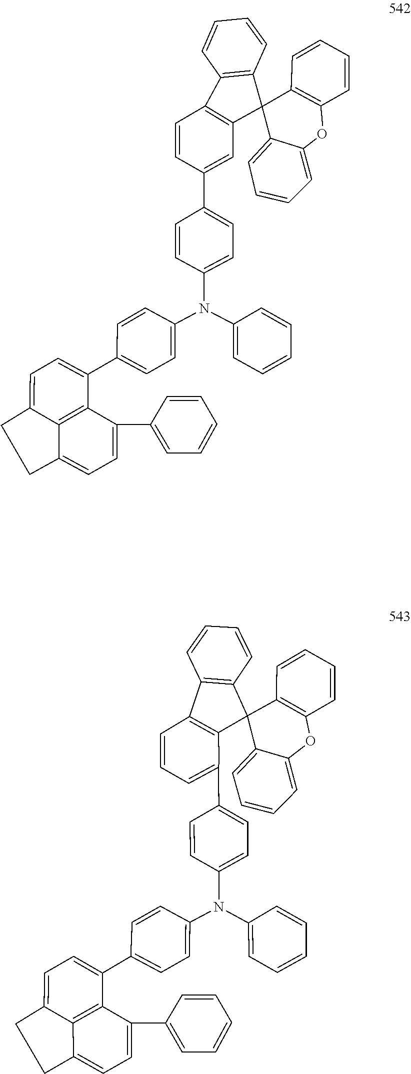 [SMILES: c1ccc(-c2ccc3c4c(ccc(-c5ccc(N(c6ccccc6)c6ccc(-c7ccc8c(c7)C7(c9ccccc9Oc9ccccc97)c7ccccc7-8)cc6)cc5)c24)CC3)cc1 ; C1=CC2=C(CC(c3ccc(N(c4ccccc4)c4ccc(-c5ccc6c7c(ccc(-c8ccccc8)c57)CC6)cc4)cc3)=C1)C1(c3ccccc3Oc3ccccc31)c1ccccc12]

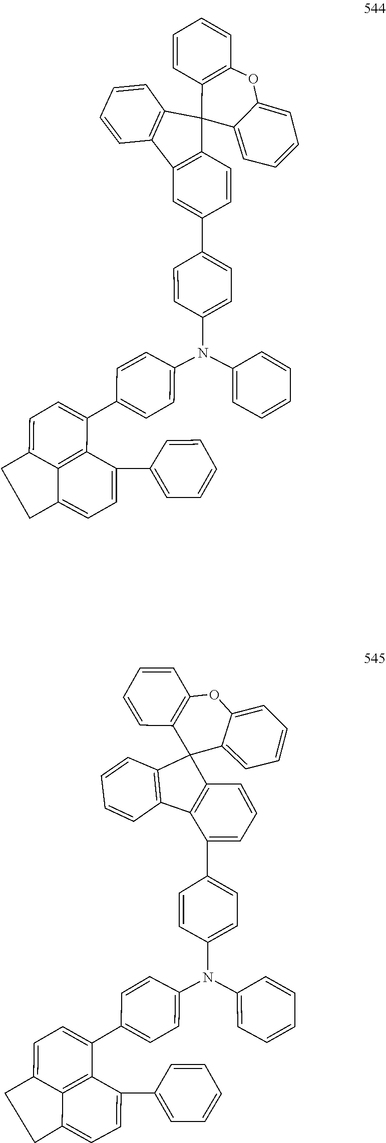 [SMILES: c1ccc(-c2ccc3c4c(ccc(-c5ccc(N(c6ccccc6)c6ccc(-c7ccc8c(c7)-c7ccccc7C87c8ccccc8Oc8ccccc87)cc6)cc5)c24)CC3)cc1 ; c1ccc(-c2ccc3c4c(ccc(-c5ccc(N(c6ccccc6)c6ccc(-c7cccc8c7-c7ccccc7C87c8ccccc8Oc8ccccc87)cc6)cc5)c24)CC3)cc1]

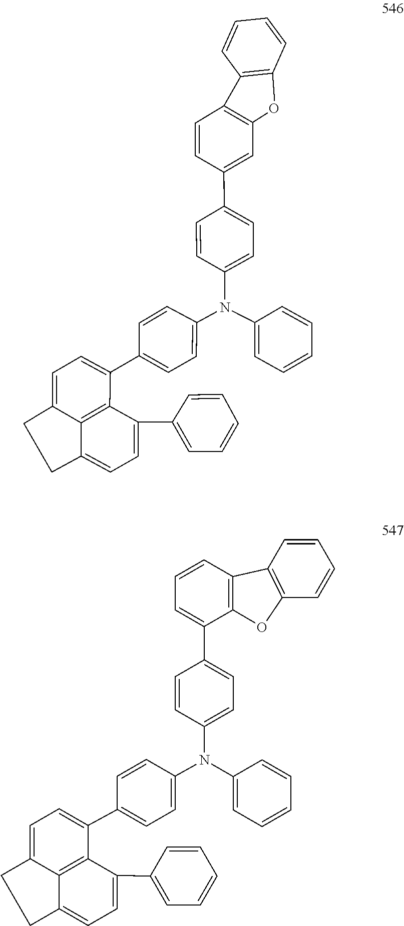 [SMILES: c1ccc(-c2ccc3c4c(ccc(-c5ccc(N(c6ccccc6)c6ccc(-c7ccc8c(c7)oc7ccccc78)cc6)cc5)c24)CC3)cc1 ; c1ccc(-c2ccc3c4c(ccc(-c5ccc(N(c6ccccc6)c6ccc(-c7cccc8c7oc7ccccc78)cc6)cc5)c24)CC3)cc1]

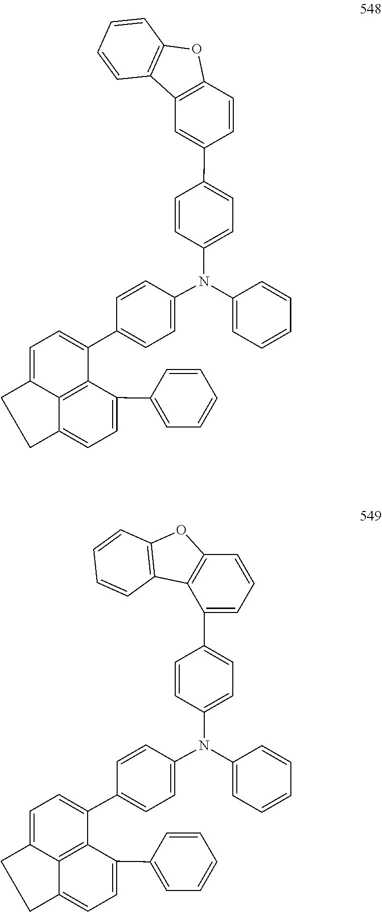 [SMILES: c1ccc(-c2ccc3c4c(ccc(-c5ccc(N(c6ccccc6)c6ccc(-c7ccc8oc9ccccc9c8c7)cc6)cc5)c24)CC3)cc1 ; c1ccc(-c2ccc3c4c(ccc(-c5ccc(N(c6ccccc6)c6ccc(-c7cccc8oc9ccccc9c78)cc6)cc5)c24)CC3)cc1]

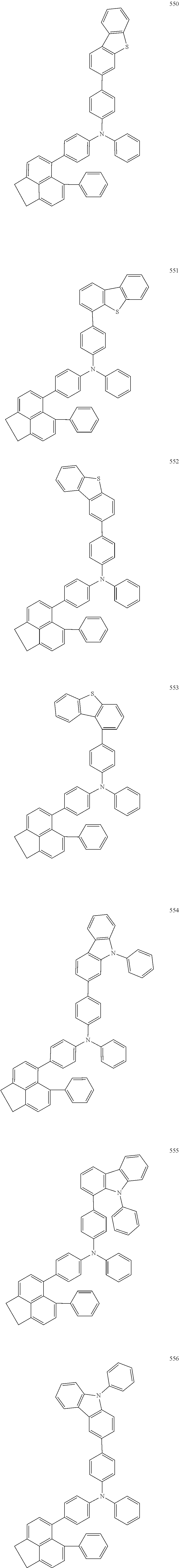

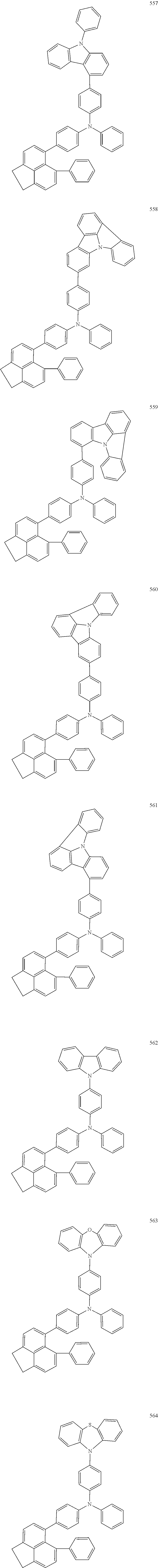

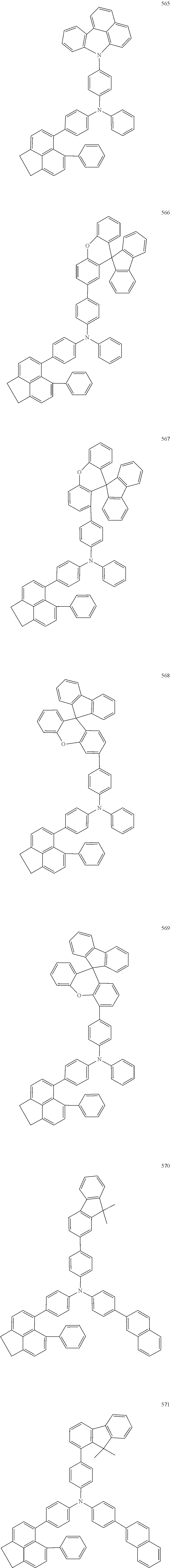

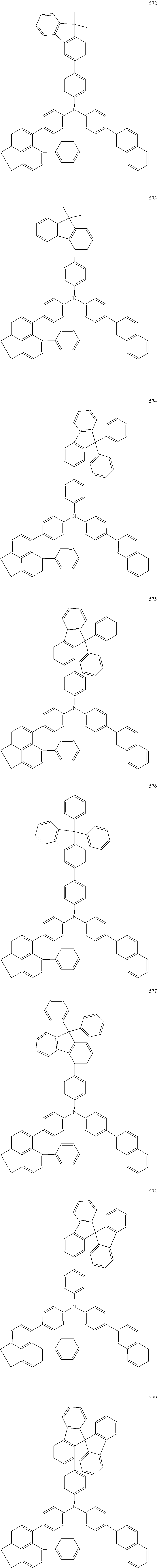

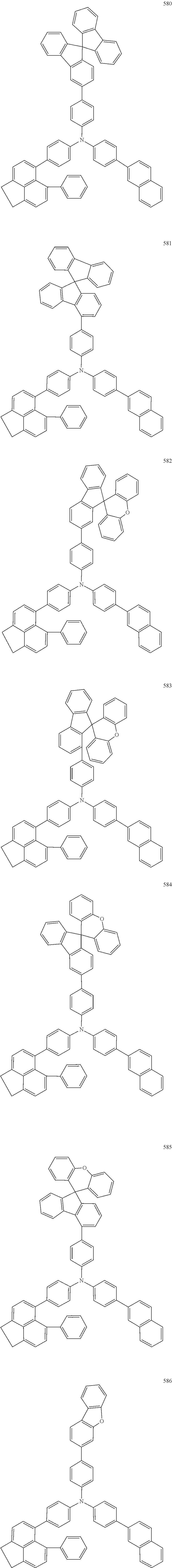

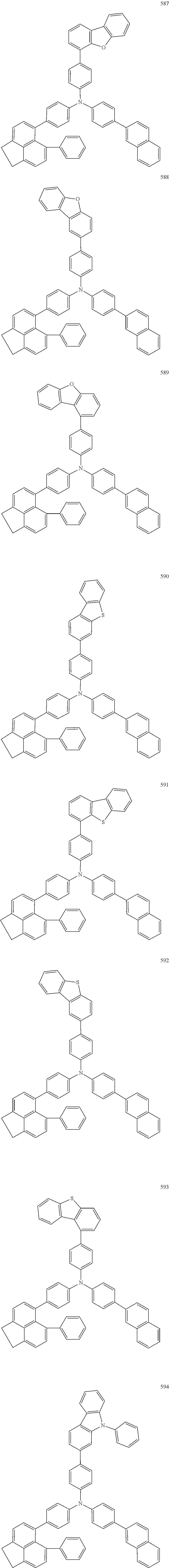

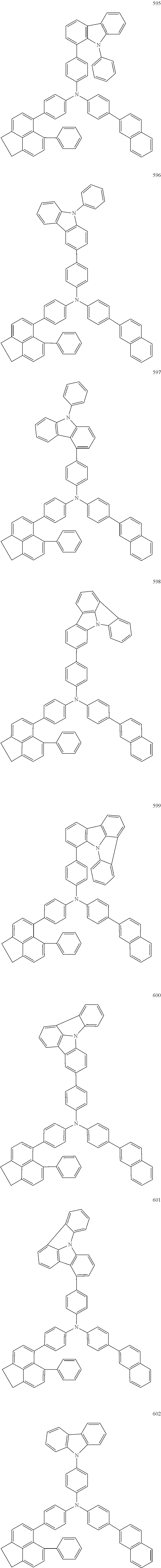

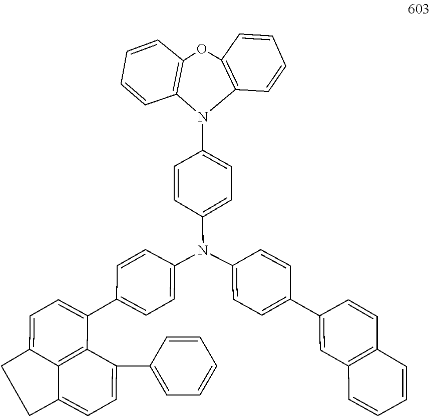 [SMILES: c1ccc(-c2ccc3c4c(ccc(-c5ccc(N(c6ccc(-c7ccc8ccccc8c7)cc6)c6ccc(N7c8ccccc8Oc8ccccc87)cc6)cc5)c24)CC3)cc1]

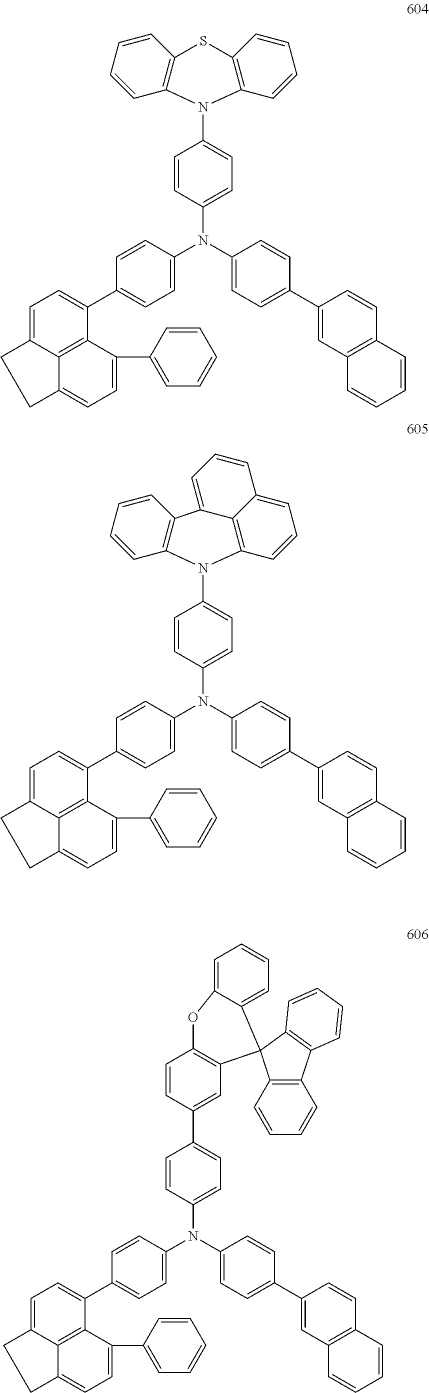 [SMILES: c1ccc(-c2ccc3c4c(ccc(-c5ccc(N(c6ccc(-c7ccc8ccccc8c7)cc6)c6ccc(N7c8ccccc8Sc8ccccc87)cc6)cc5)c24)CC3)cc1 ; c1ccc(-c2ccc3c4c(ccc(-c5ccc(N(c6ccc(-c7ccc8ccccc8c7)cc6)c6ccc(N7c8ccccc8-c8cccc9cccc7c89)cc6)cc5)c24)CC3)cc1 ; c1ccc(-c2ccc3c4c(ccc(-c5ccc(N(c6ccc(-c7ccc8c(c7)C7(c9ccccc9O8)c8ccccc8-c8ccccc87)cc6)c6ccc(-c7ccc8ccccc8c7)cc6)cc5)c24)CC3)cc1]

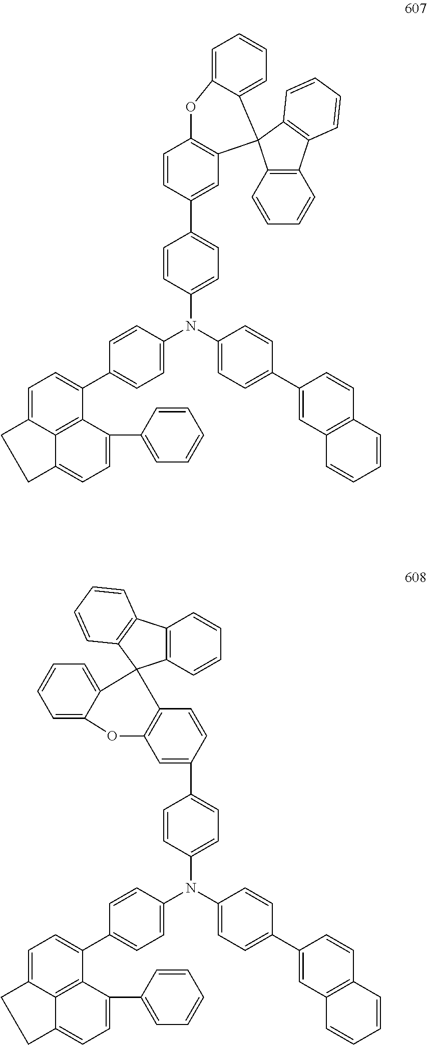 [SMILES: c1ccc(-c2ccc3c4c(ccc(-c5ccc(N(c6ccc(-c7ccc8c(c7)C7(c9ccccc9O8)c8ccccc8-c8ccccc87)cc6)c6ccc(-c7ccc8ccccc8c7)cc6)cc5)c24)CC3)cc1 ; c1ccc(-c2ccc3c4c(ccc(-c5ccc(N(c6ccc(-c7ccc8c(c7)Oc7ccccc7C87c8ccccc8-c8ccccc87)cc6)c6ccc(-c7ccc8ccccc8c7)cc6)cc5)c24)CC3)cc1]

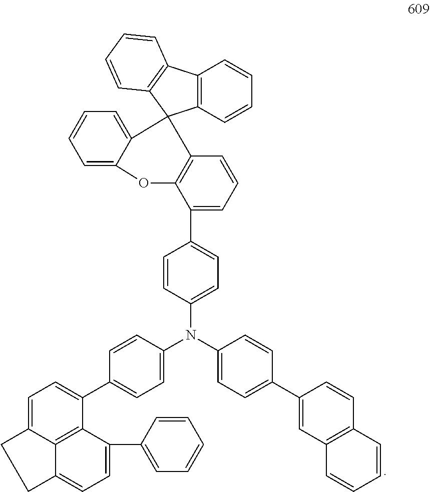 [SMILES: c1ccc(-c2ccc3c4c(ccc(-c5ccc(N(c6ccc(-c7ccc8ccccc8c7)cc6)c6ccc(-c7cccc8c7Oc7ccccc7C87c8ccccc8-c8ccccc87)cc6)cc5)c24)CC3)cc1]

8. The aromatic amine compound according to claim 1, wherein the aromatic amine compounds represented by Chemical Formula II or Chemical Formula III: 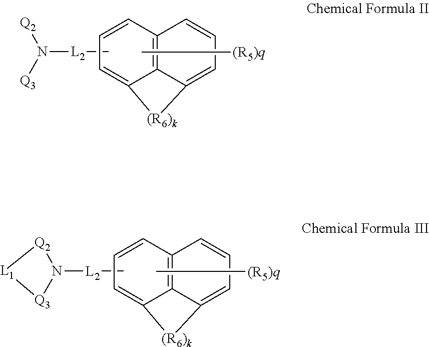
wherein, L₂ is selected from a single bond, a substituted or unsubstituted aryl group having 6 to 30 carbon atoms, or a substituted or unsubstituted heteroaryl group having 3 to 30 carbon atoms.

9. The aromatic amine compound according to claim 8, wherein L₂ is selected from a single bond, substituted or unsubstituted phenylene, substituted or unsubstituted biphenylene, or substituted or unsubstituted terphenylene.

10. The aromatic amine compound according to claim 9, wherein L₂ is substituted with at least one substituent, the substituent(s) on L₂ are selected from deuterium, aryl groups having 6 to 12 carbon atoms, heteroaryl groups having 3 to 12 carbon atoms, alkyl groups having 1 to 4 carbon atoms; when the number of substituents on L₂ is plural, any two substituents are the same or different.

11. The aromatic amine compound according to claim 8, wherein Q₂ is selected from 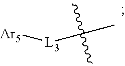
Q₃ is selected from 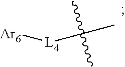
Ar₅ and Ar₆ are the same or different, and each is independently selected from a substituted or unsubstituted group W1. The unsubstituted group W1 is selected from: 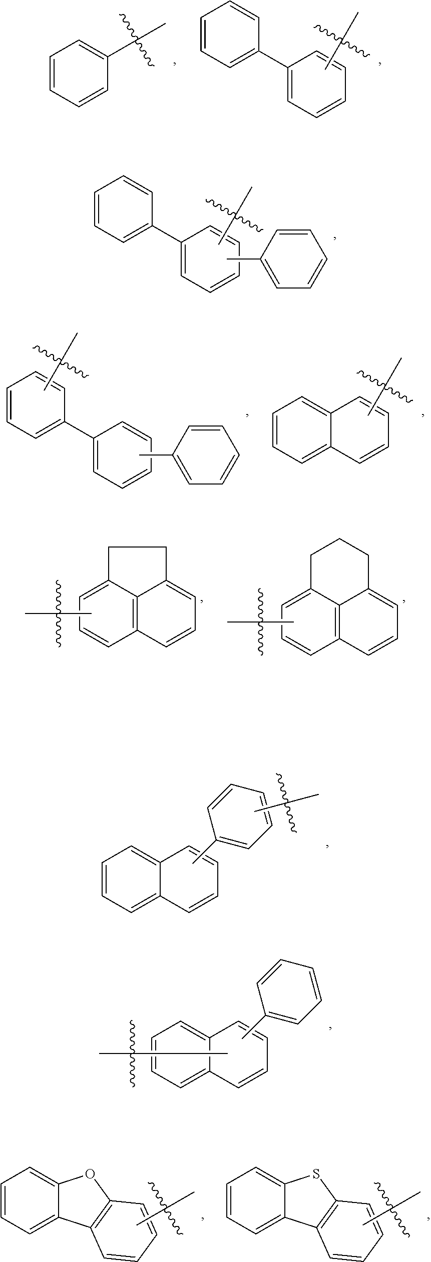 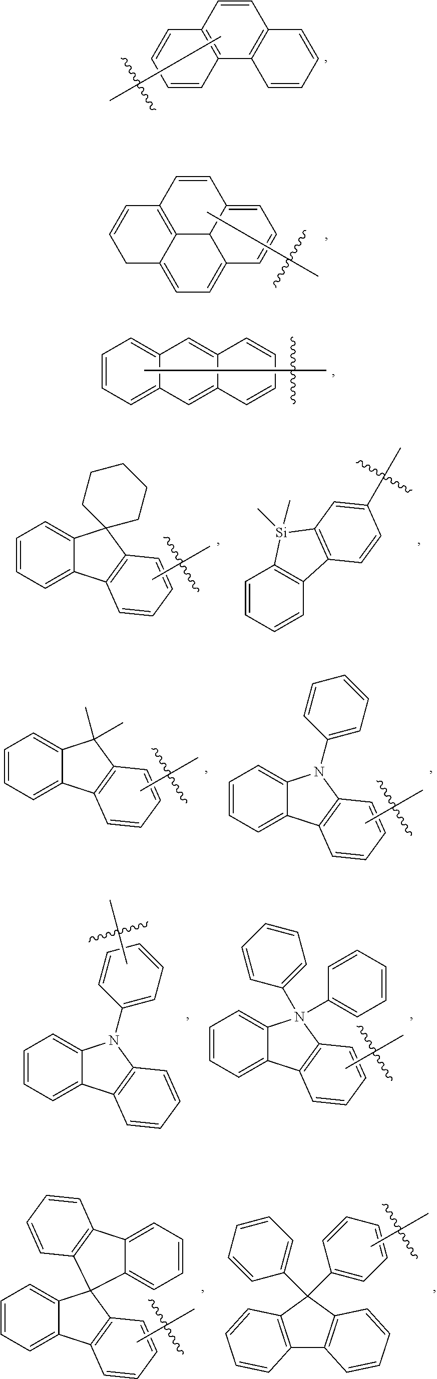 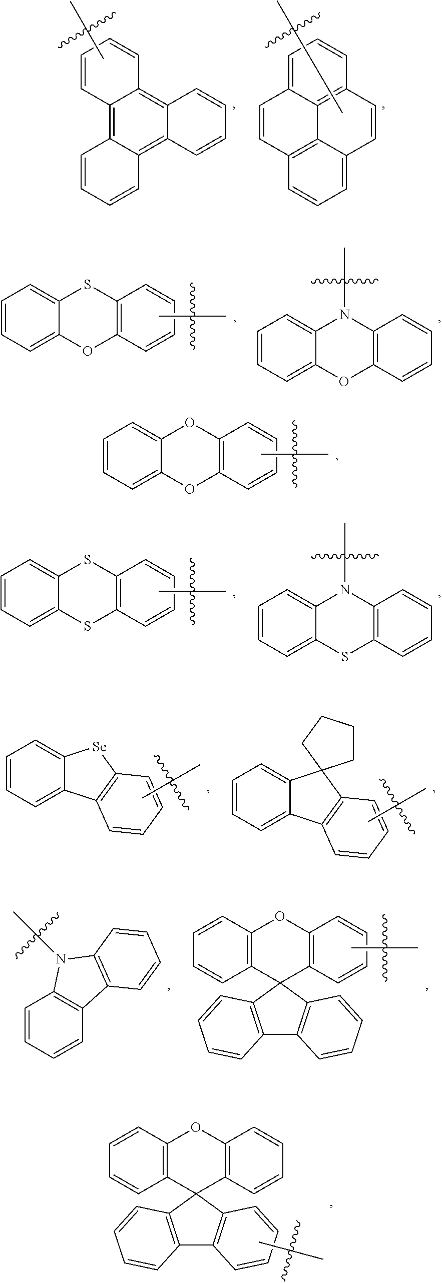 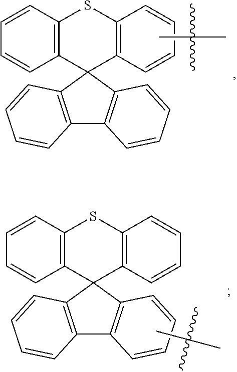
wherein, the substituted group W1 is formed by replacing one or more hydrogens on the unsubstituted group W1 with substituents selected from deuterium, fluorine, chlorine, bromine, an alkyl group having 1 to 6 carbon atoms, a cycloalkyl group having 5 to 10 carbon atoms, an aryl group having 6 to 20 carbon atoms, or a heteroaryl group having 3 to 20 carbon atoms. When multiple substituents are present on the substituted group W1, any two substituents are the same or different.

12. The aromatic amine compound according to claim 11, wherein, L₃ and L₄ are the same or different, and each is independently selected from a single bond or a substituted or unsubstituted group W2, and the unsubstituted group W2 is selected from: 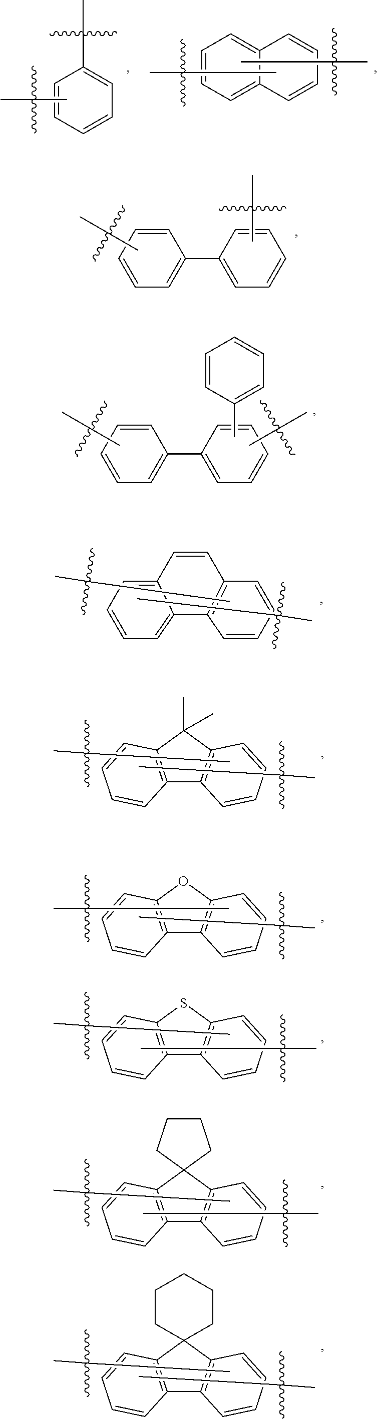 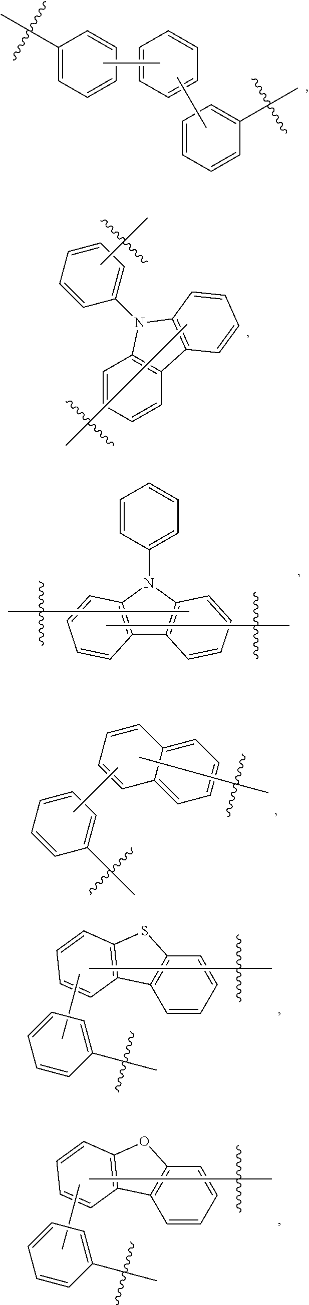 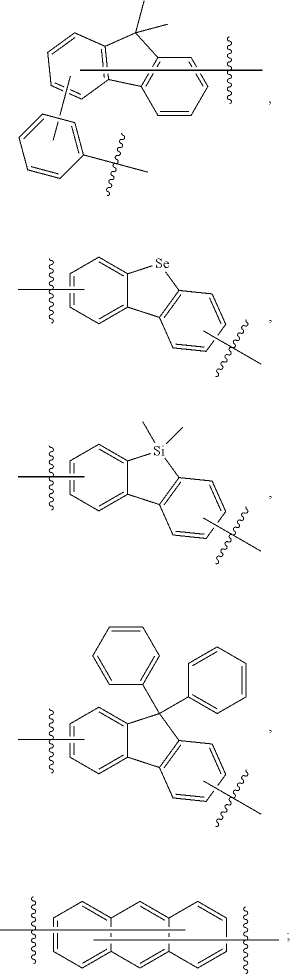
wherein, the substituted group W2 is formed by replacing one or more hydrogens on the unsubstituted group W2 with substituents selected from deuterium, fluorine, chlorine, bromine, an alkyl group having 1 to 6 carbon atoms, a cycloalkyl group having 5 to 10 carbon atoms, an aryl group having 6 to 20 carbon atoms, or a heteroaryl group having 3 to 20 carbon atoms; when the number of substituents on the substituted group W2 is plural, any two substituents may be the same or different.

13. The aromatic amine compound according to claim 8, wherein, 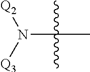
is selected from a substituted or unsubstituted group W3, and the unsubstituted group W3 is selected from Formulas a1 to a2-296 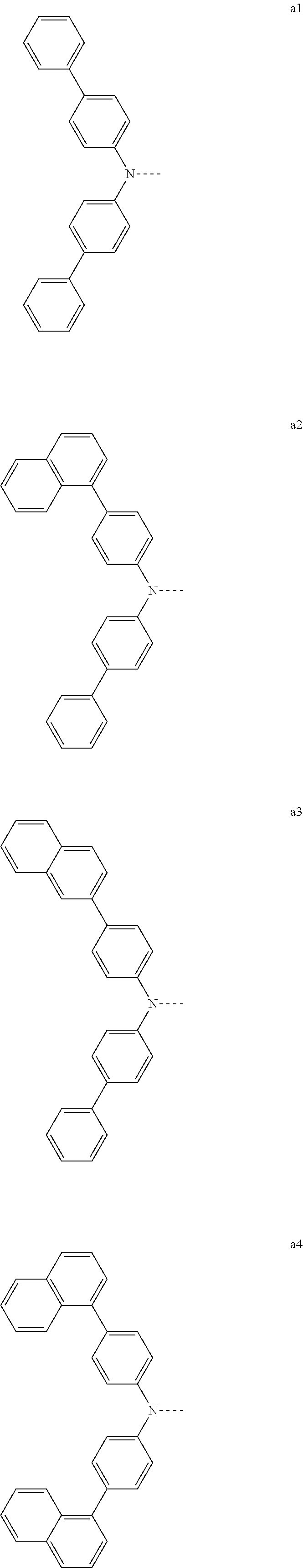 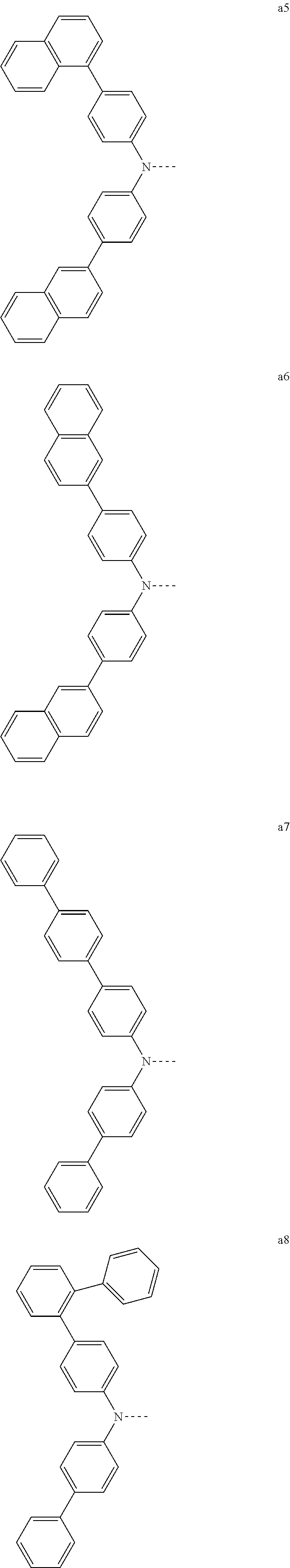 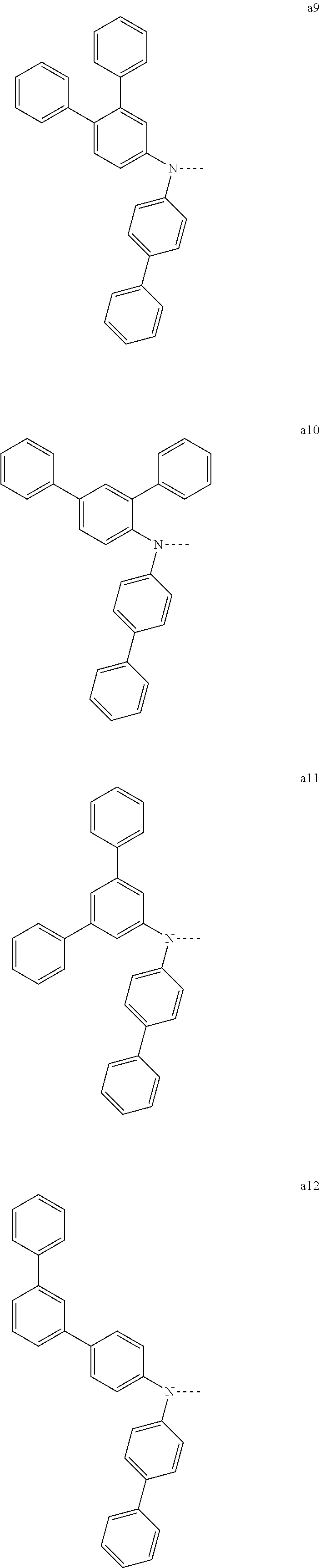 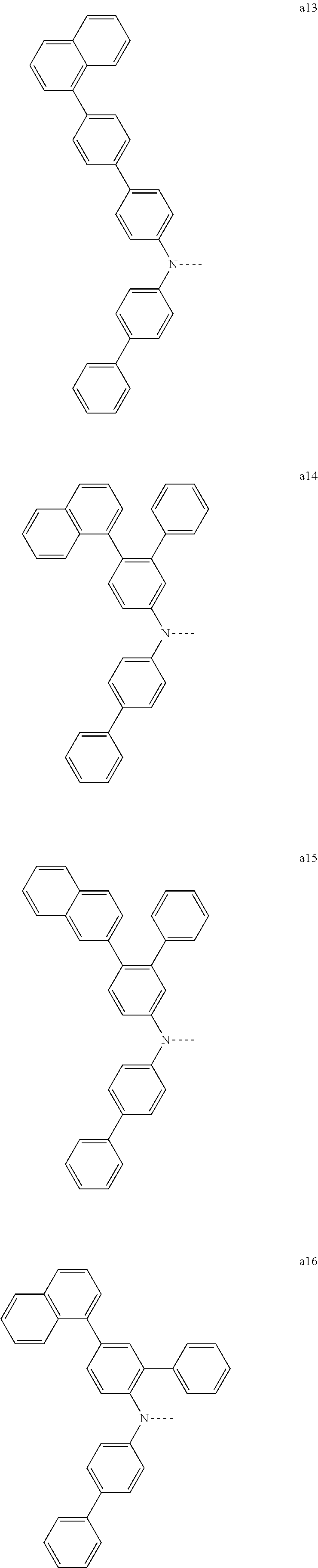 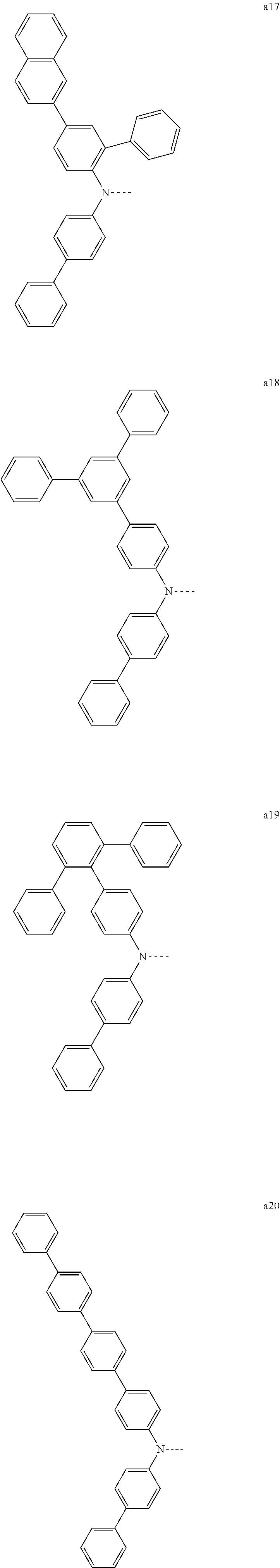 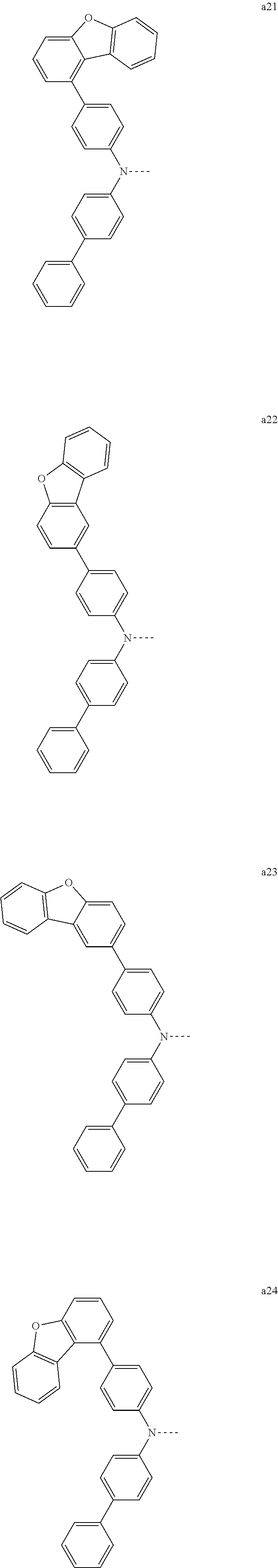 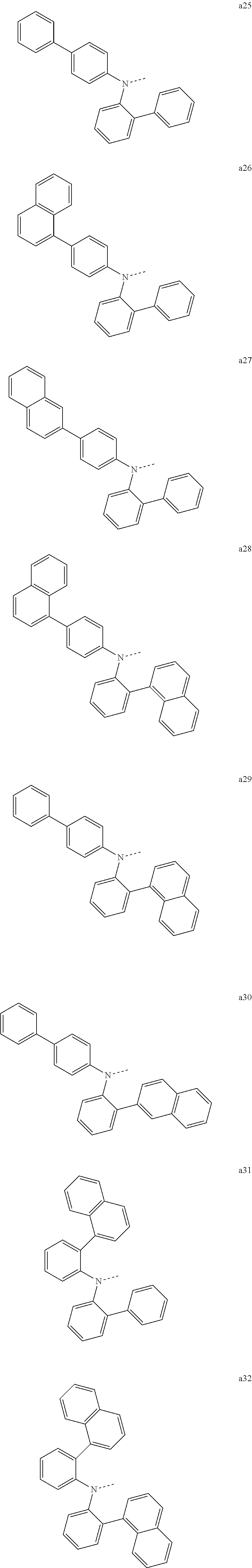 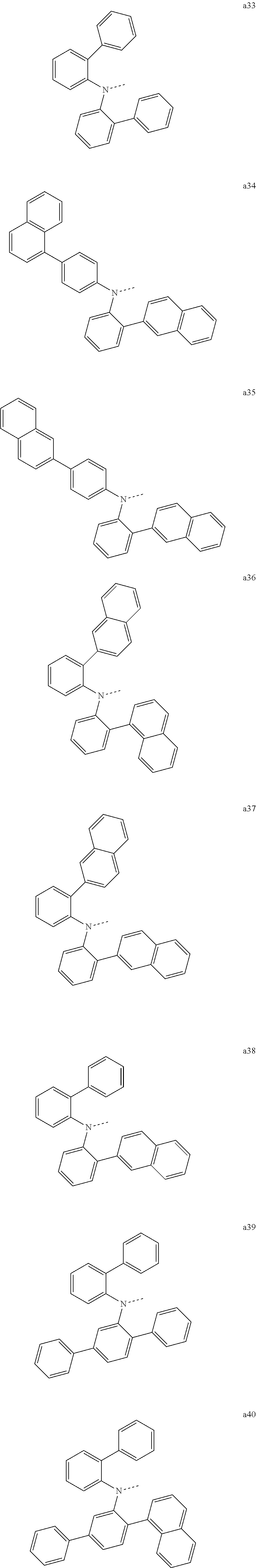 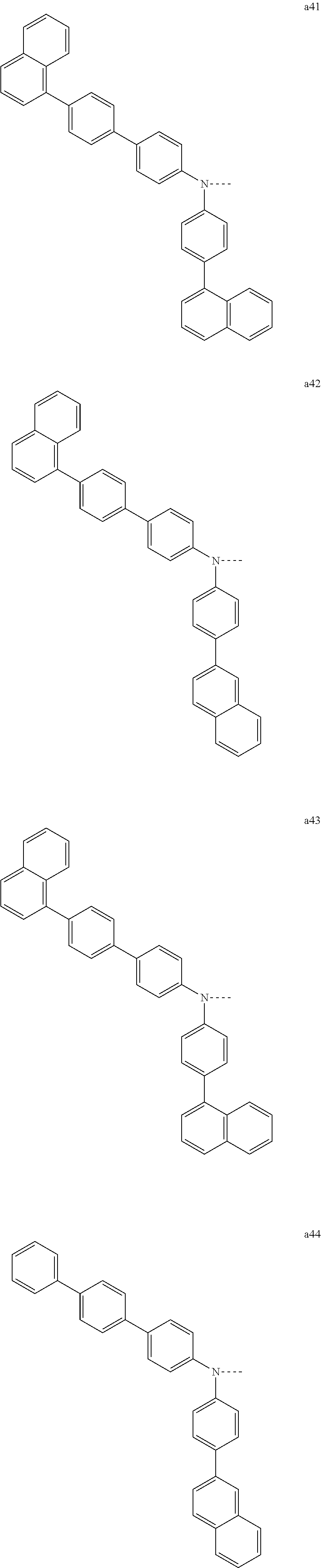 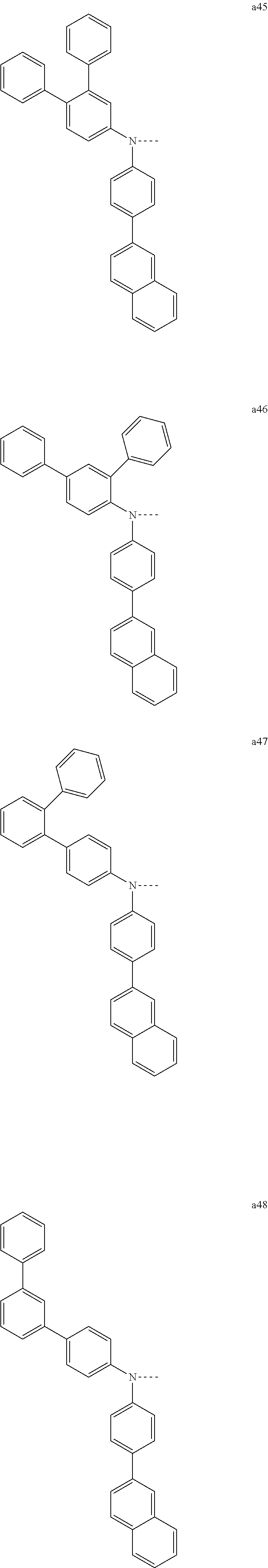 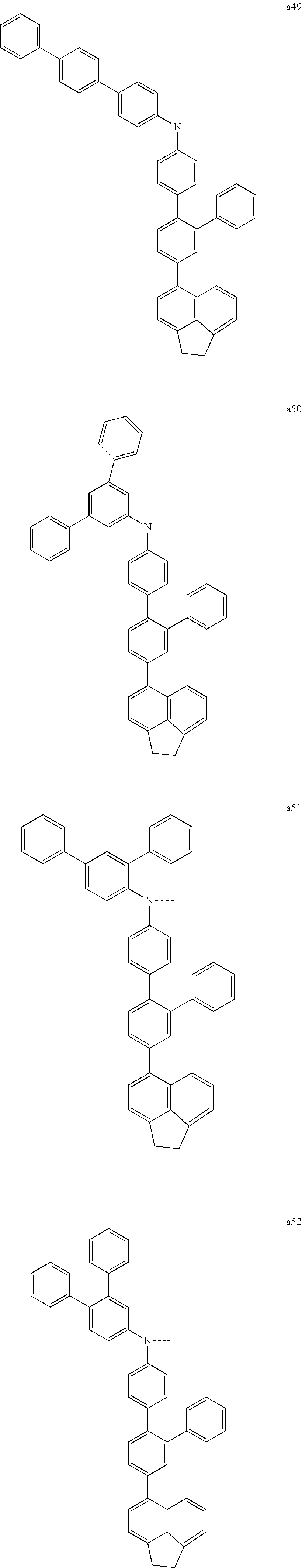 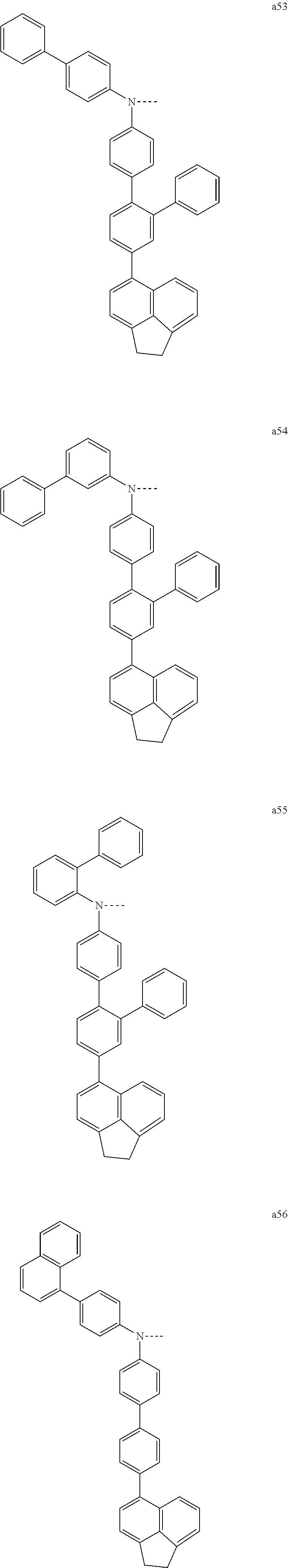 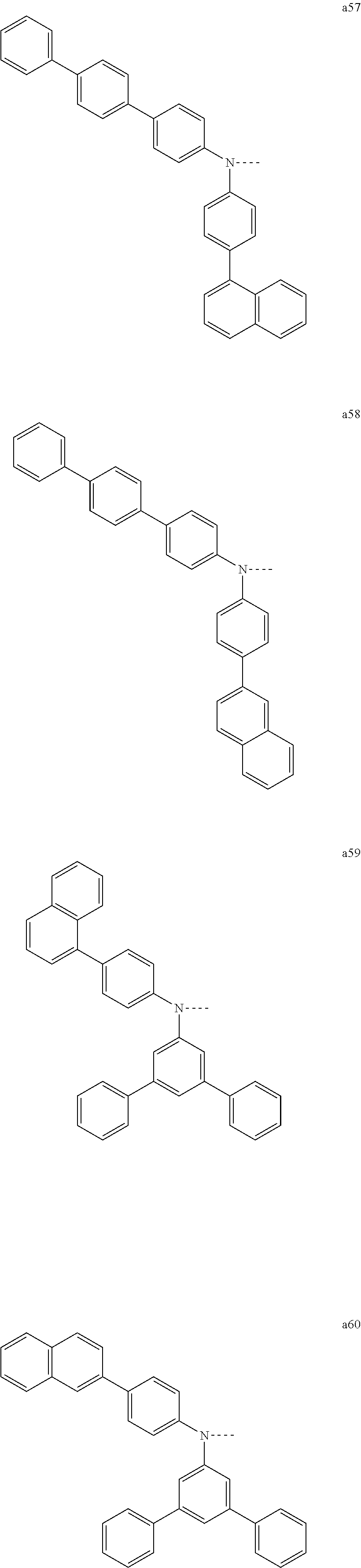 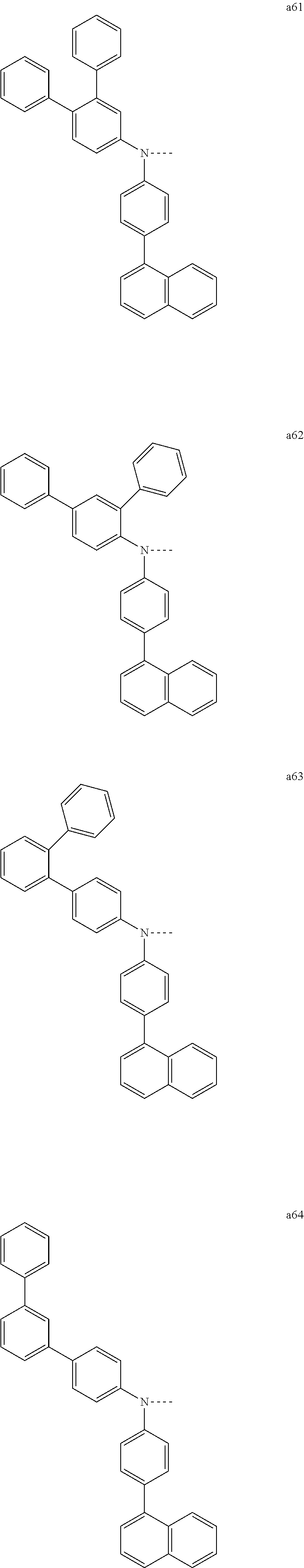 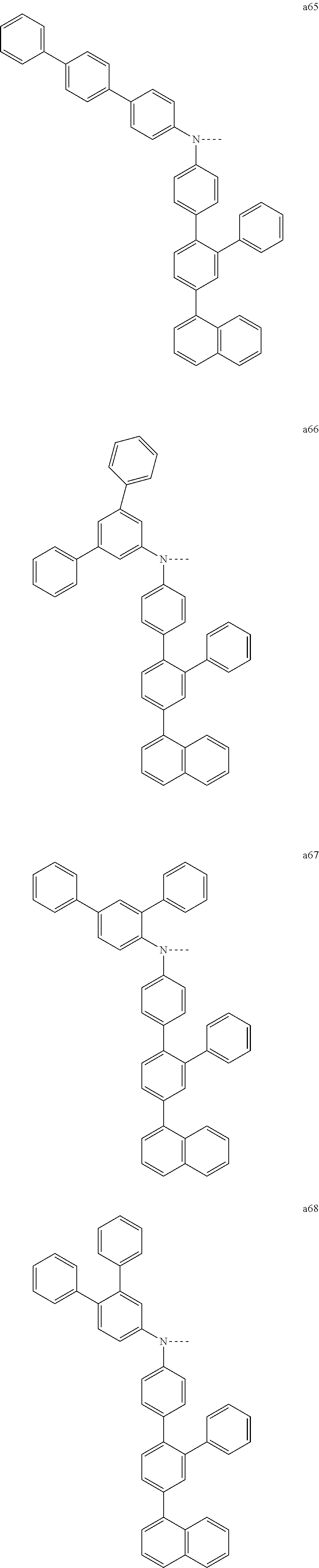 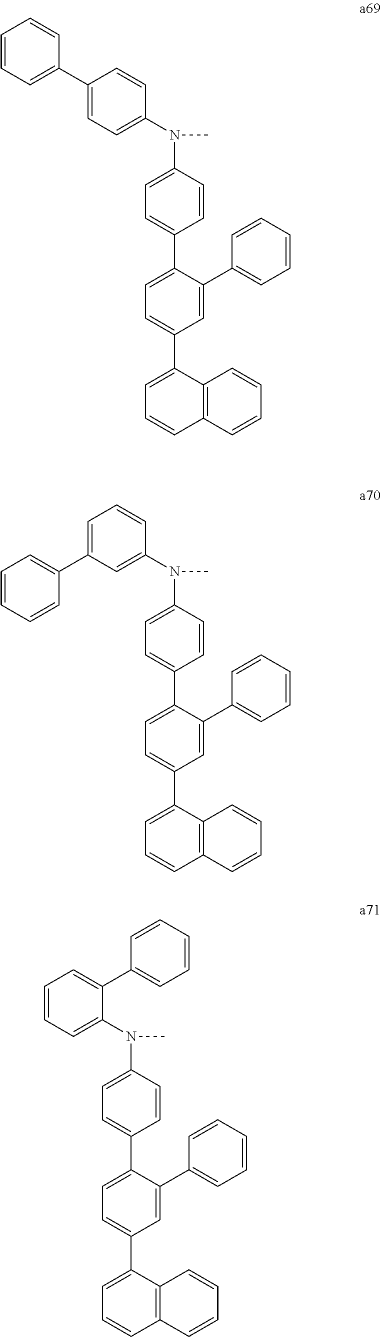 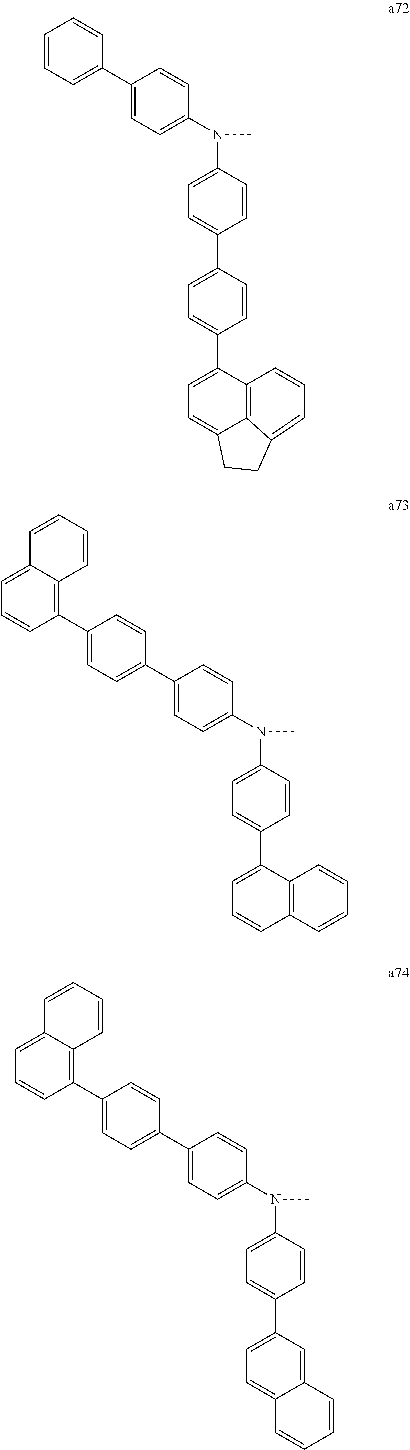 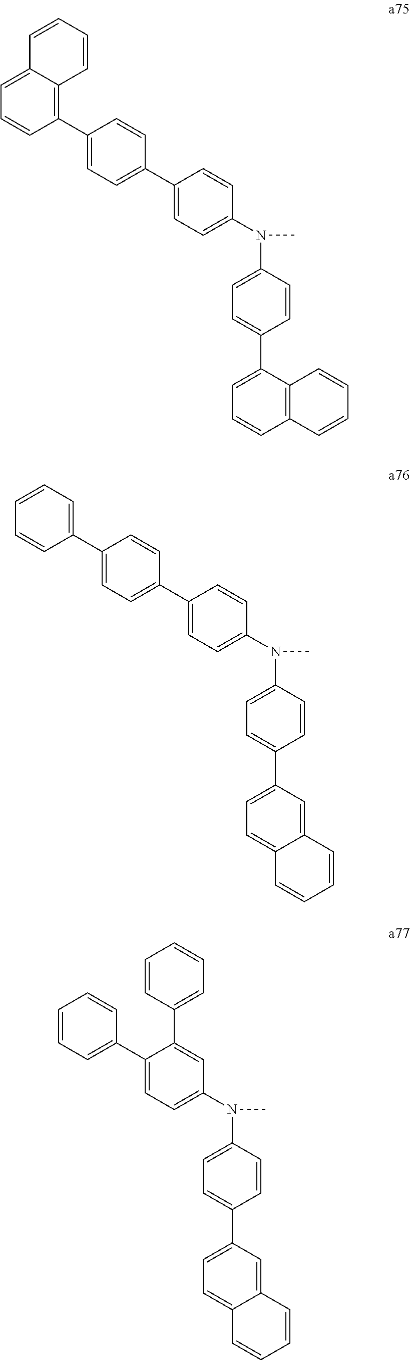 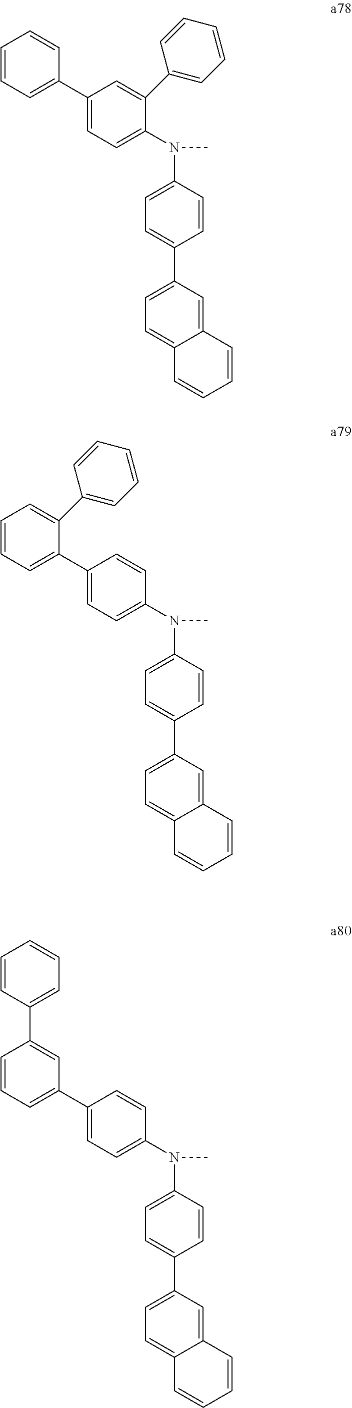 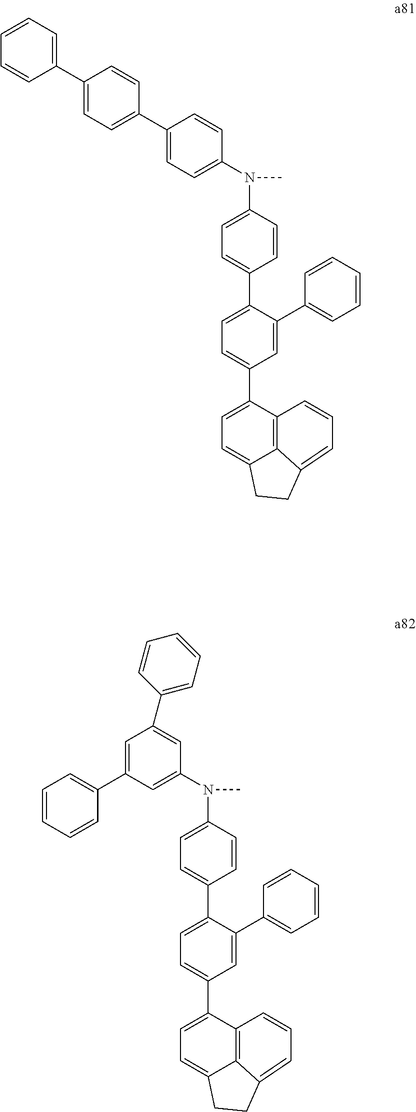 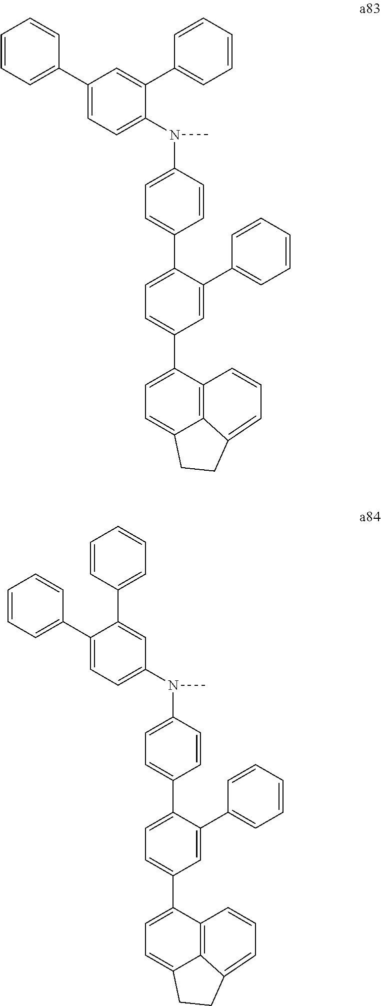 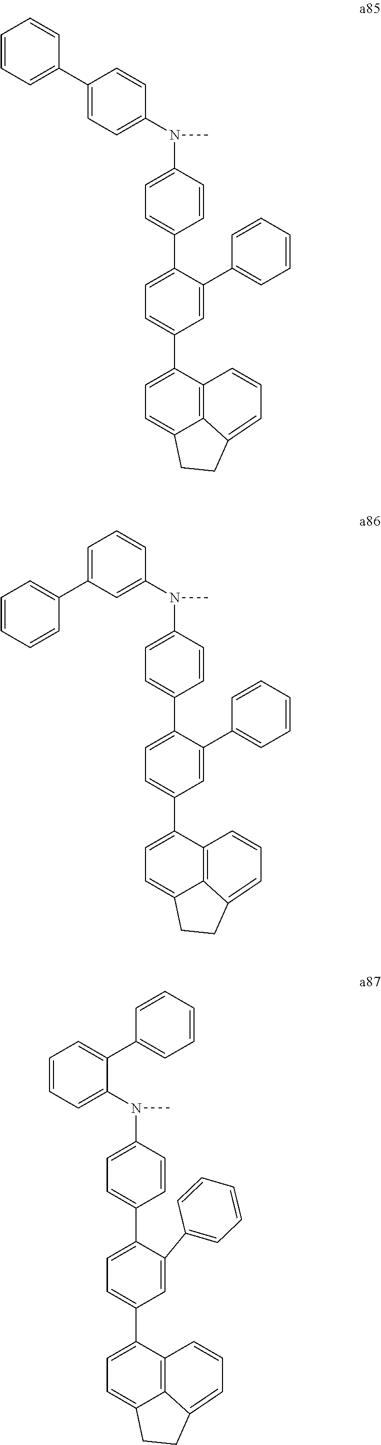 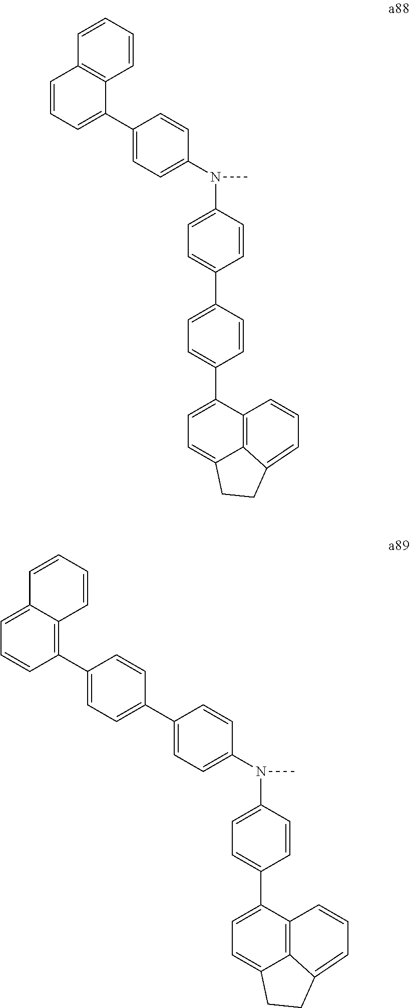 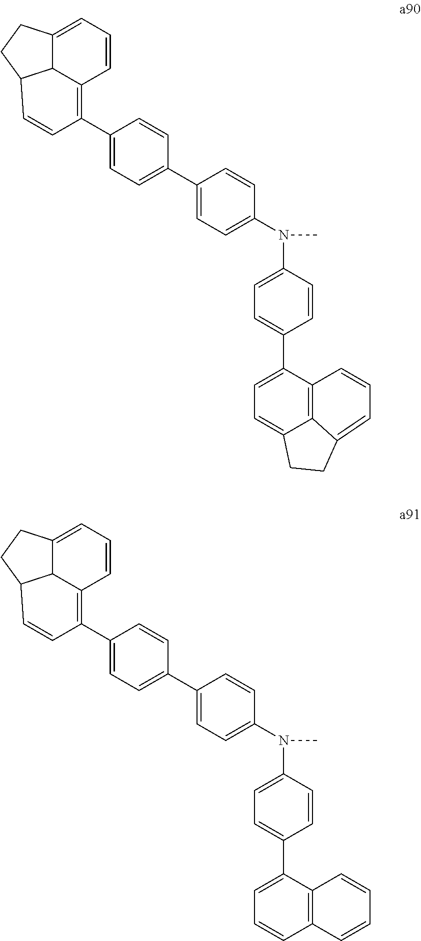 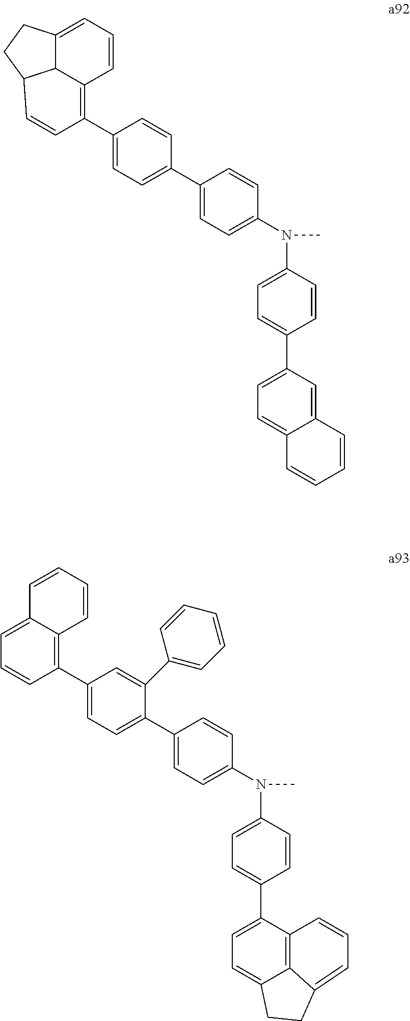 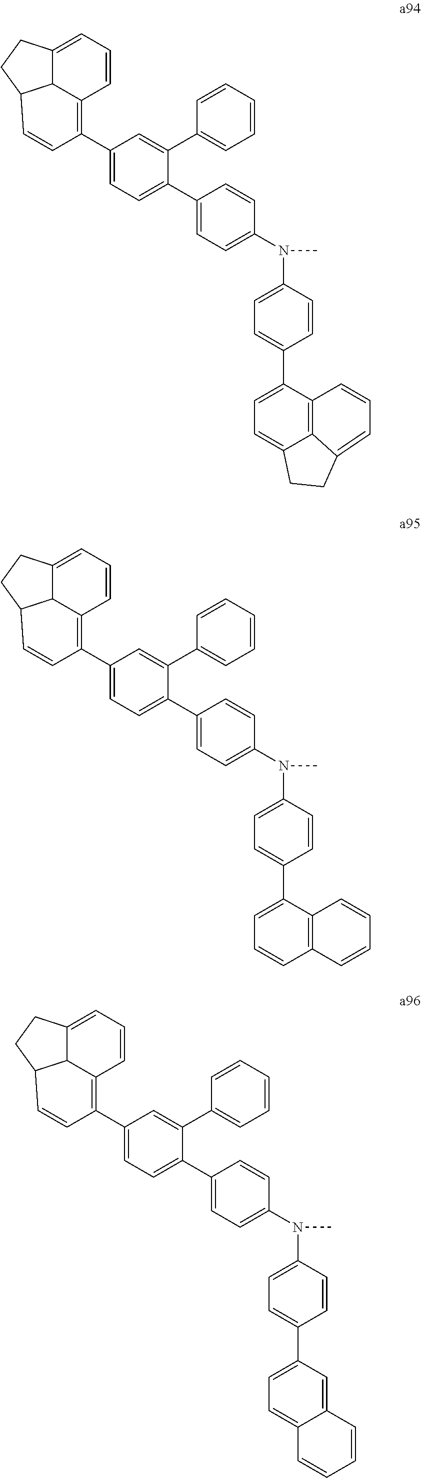 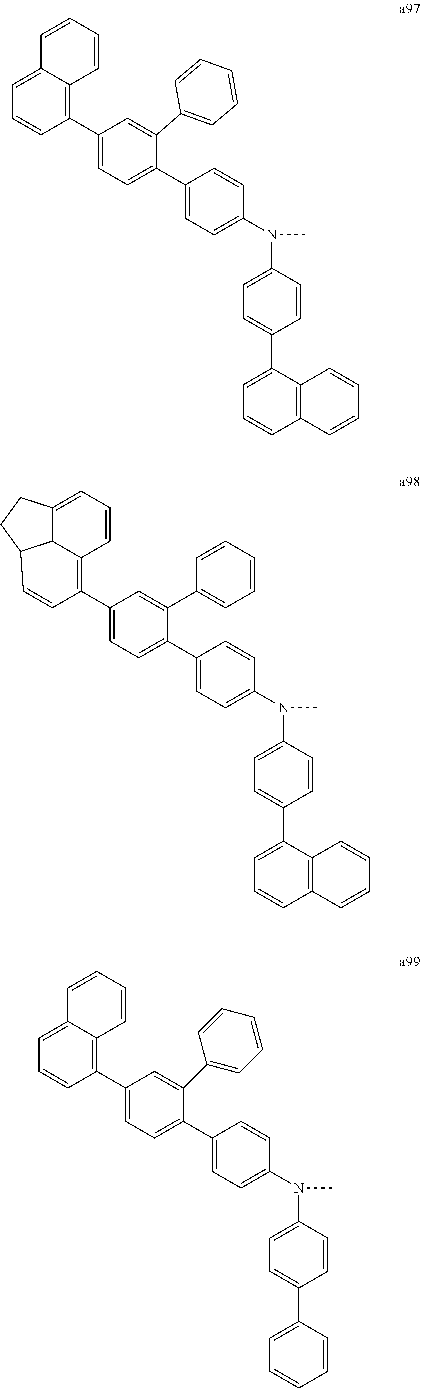 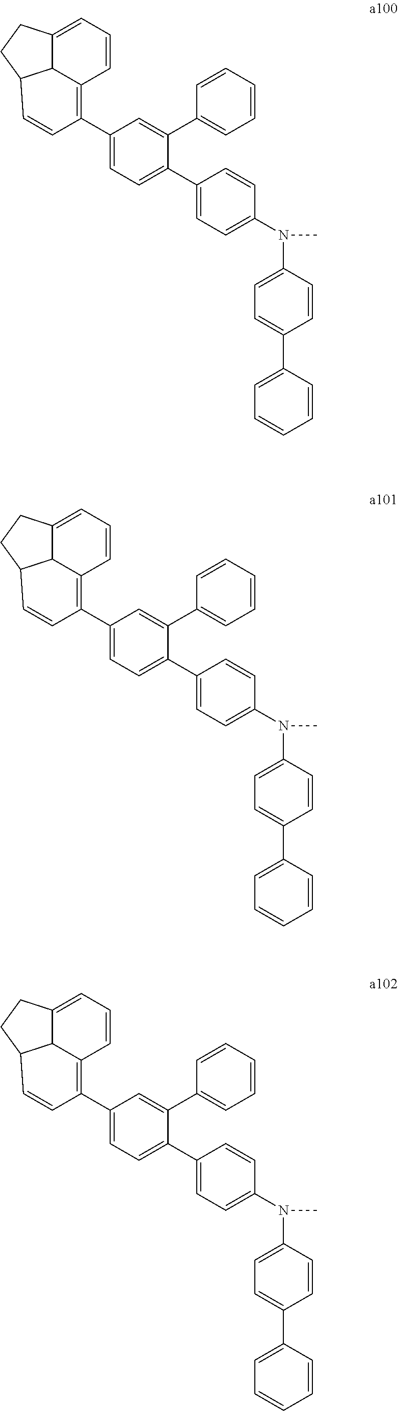 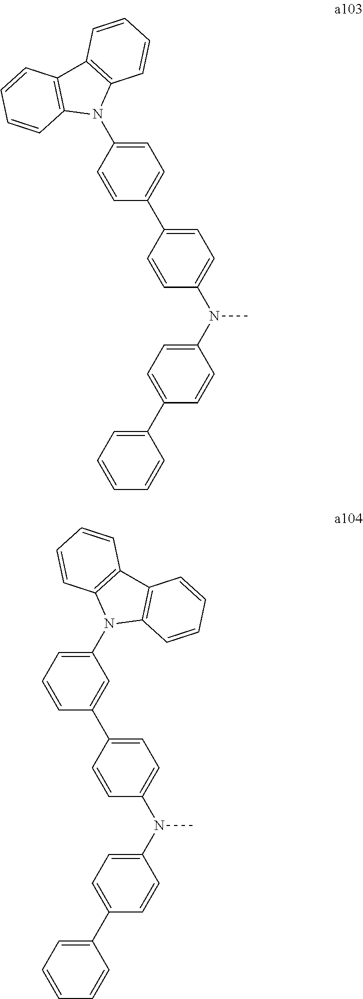 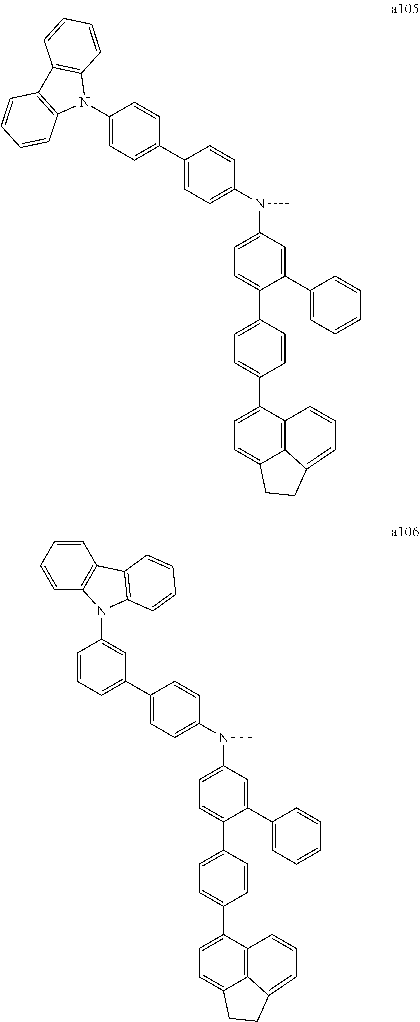 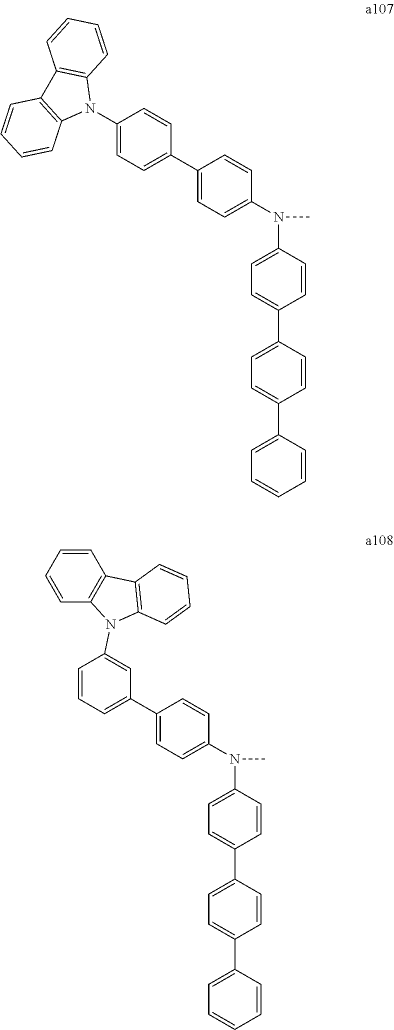 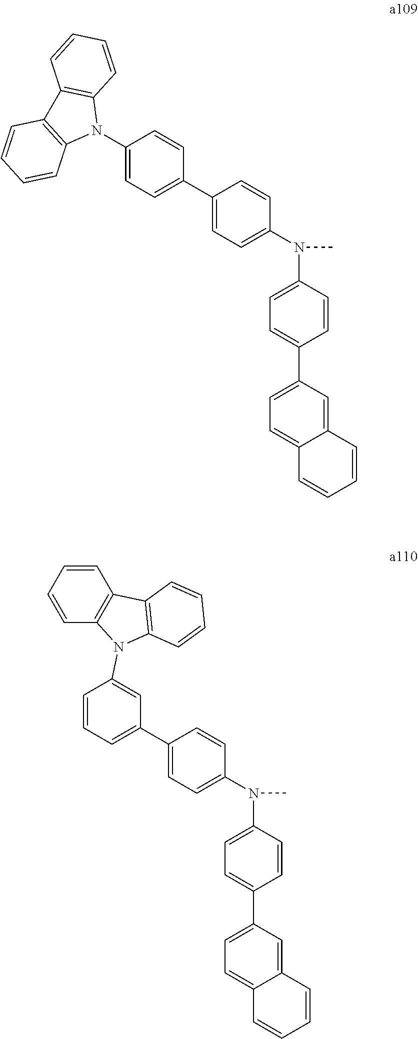 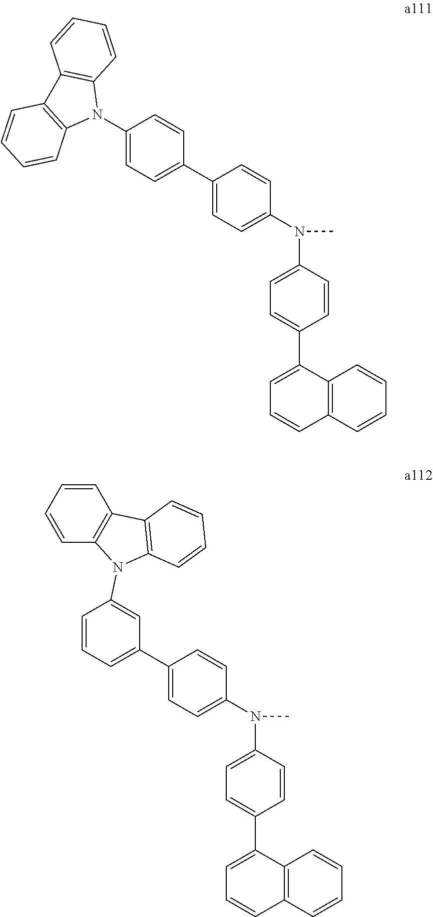 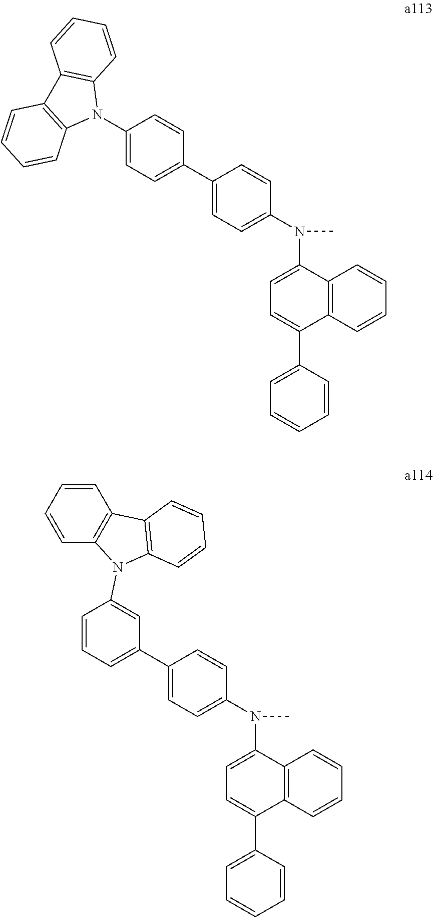 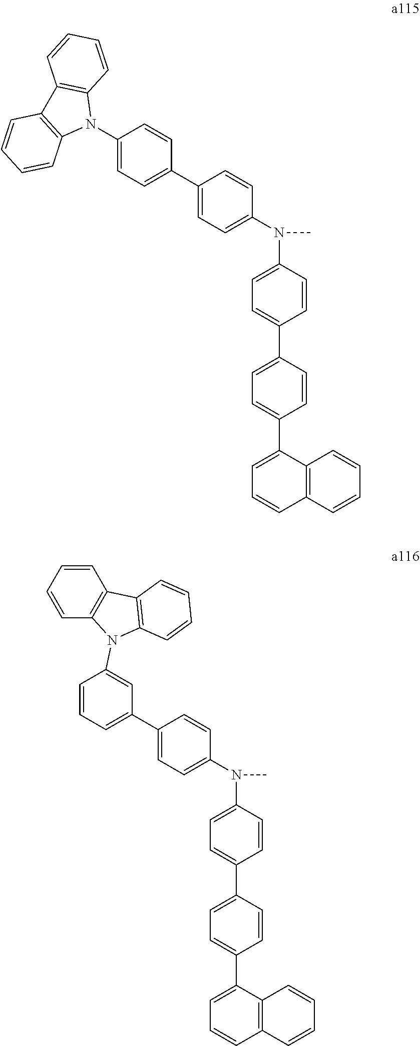 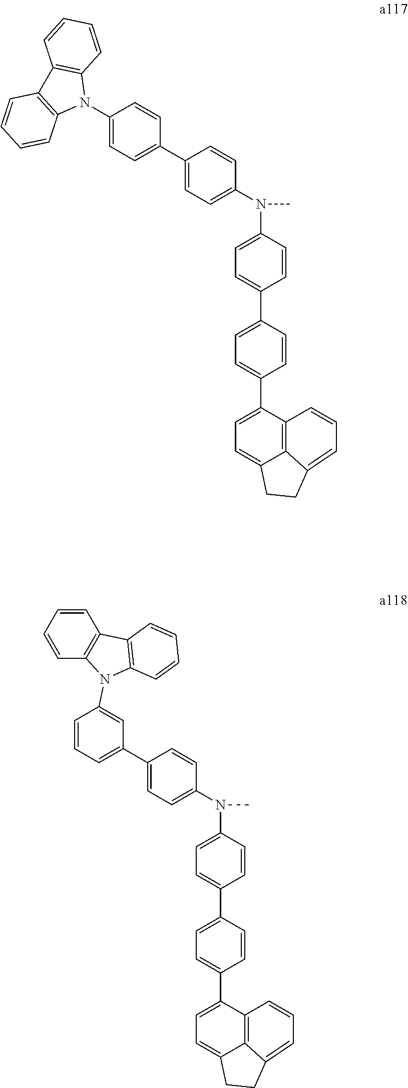 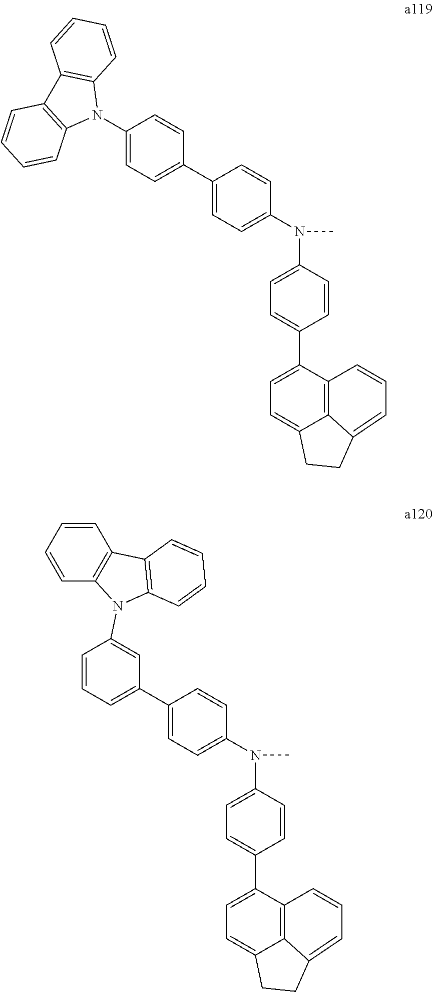 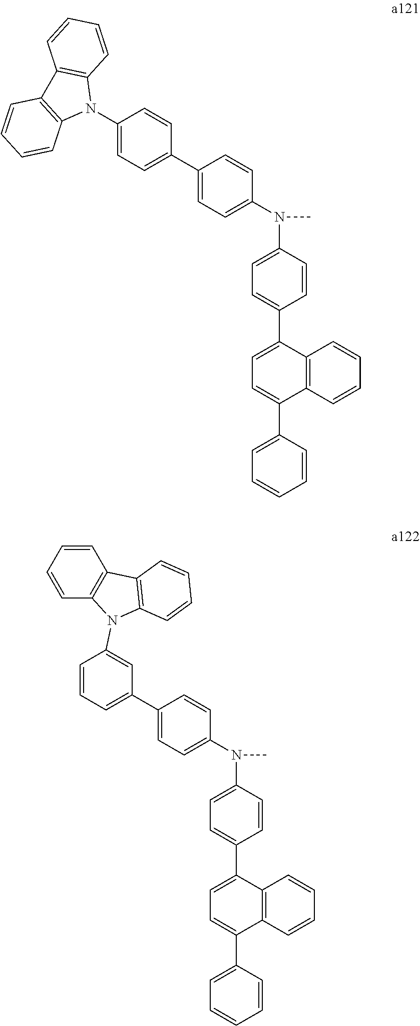 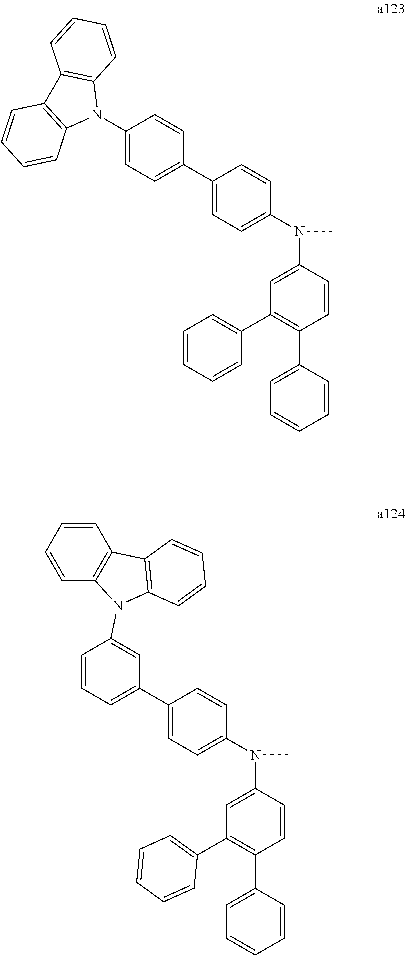 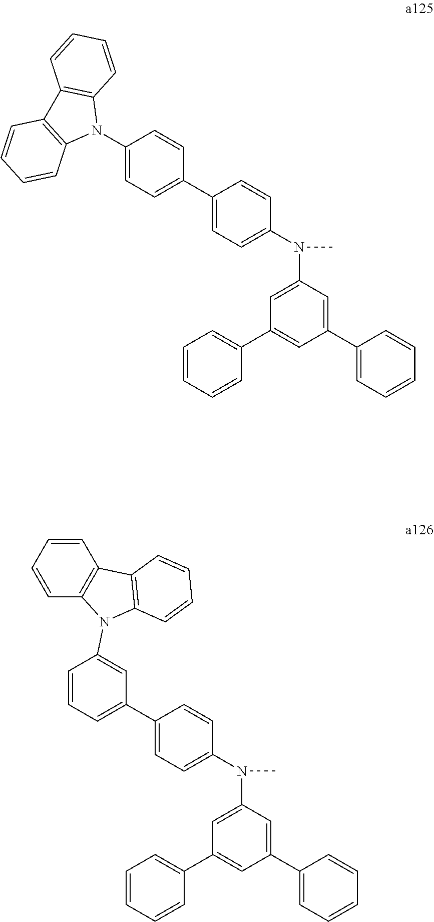 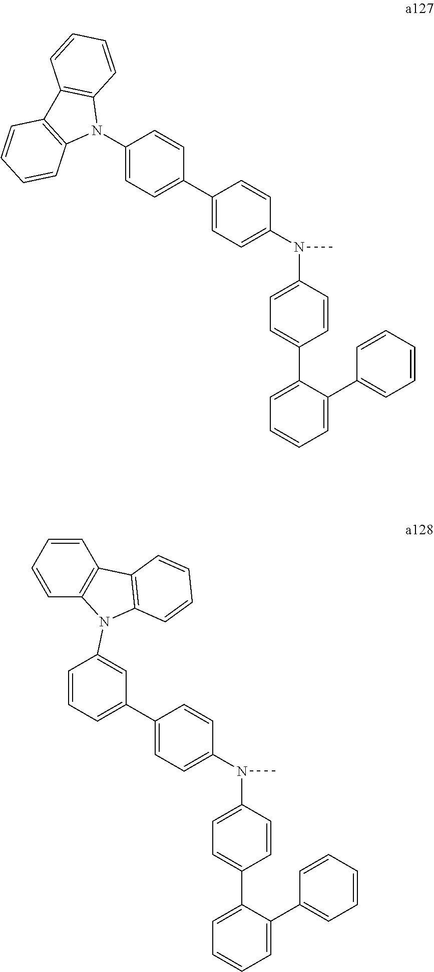 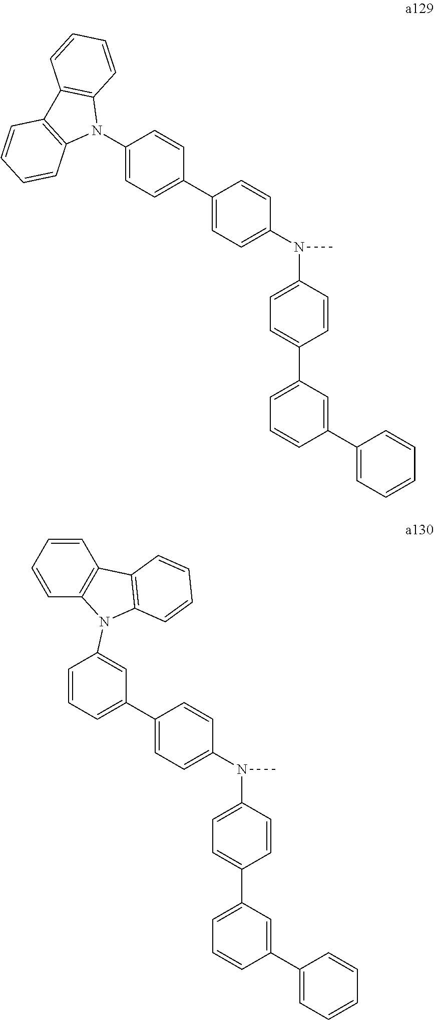 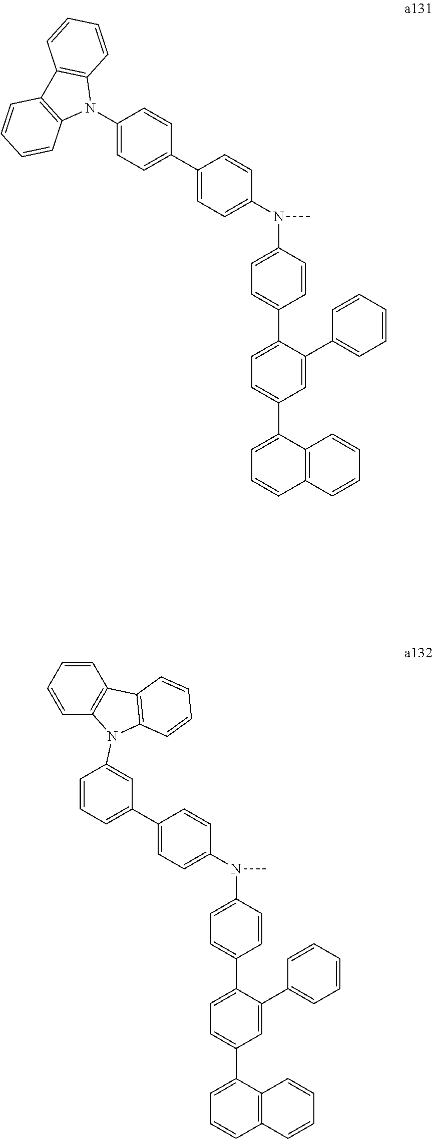 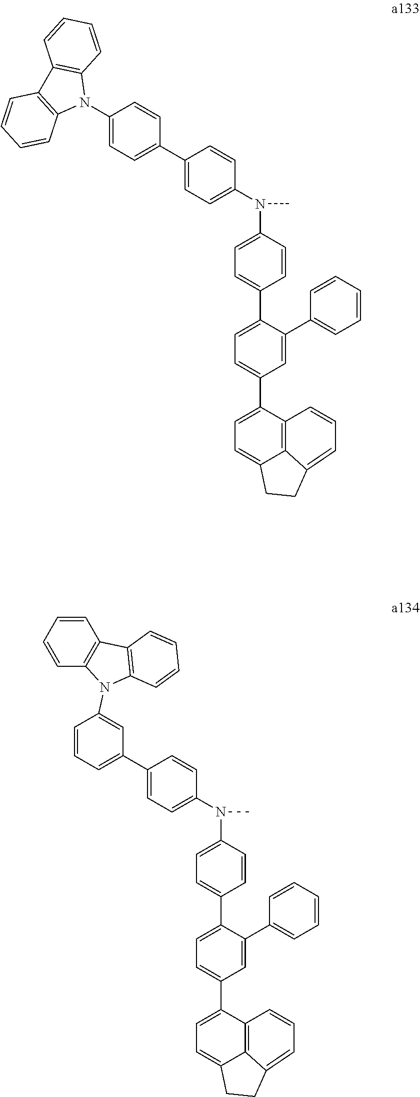 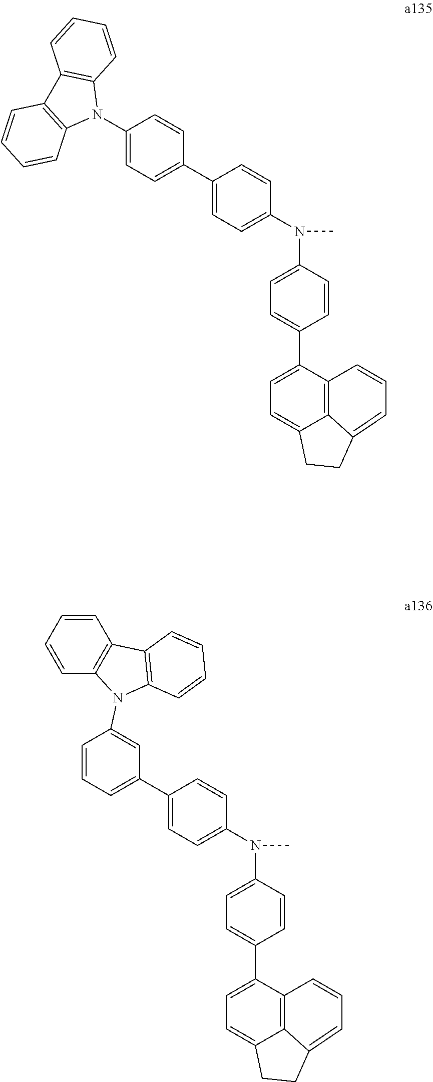 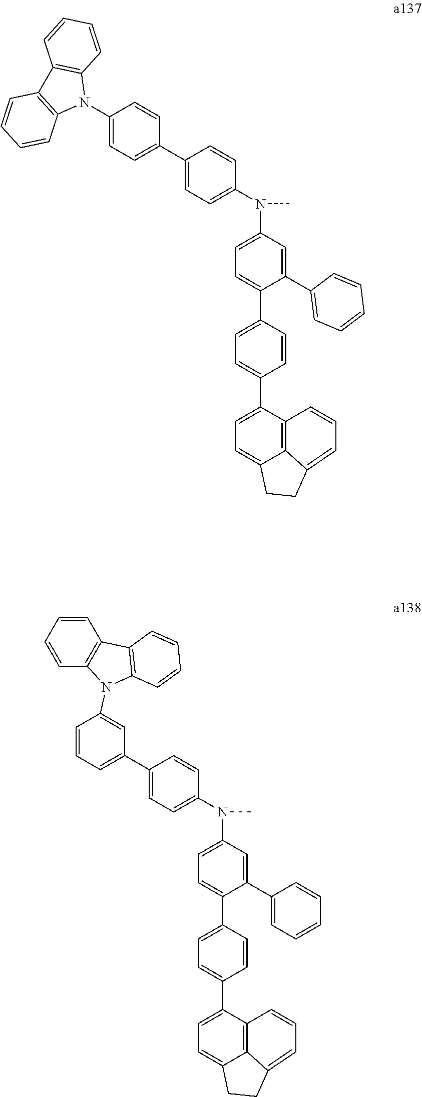 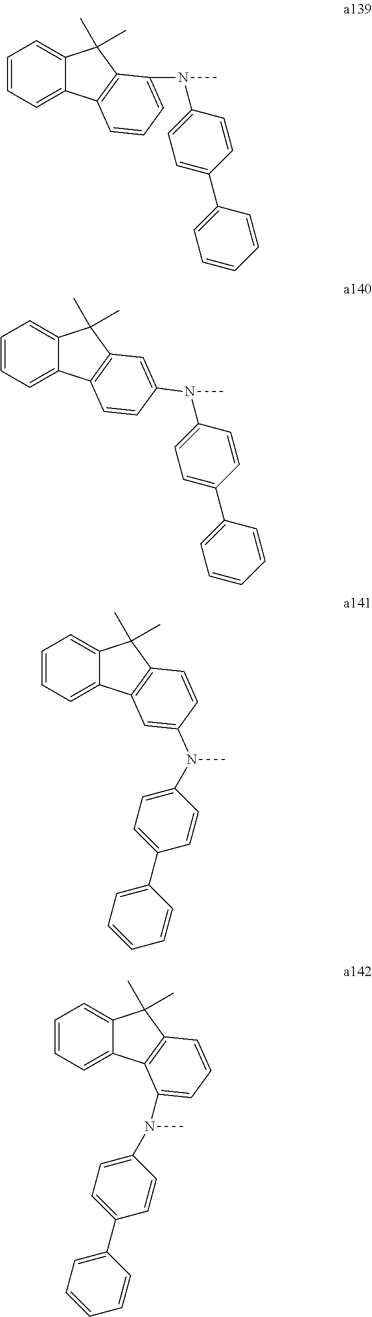 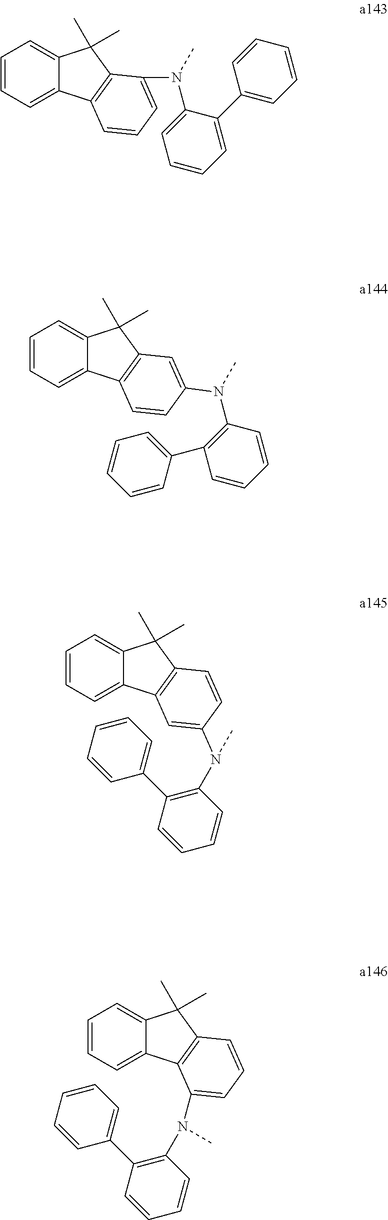 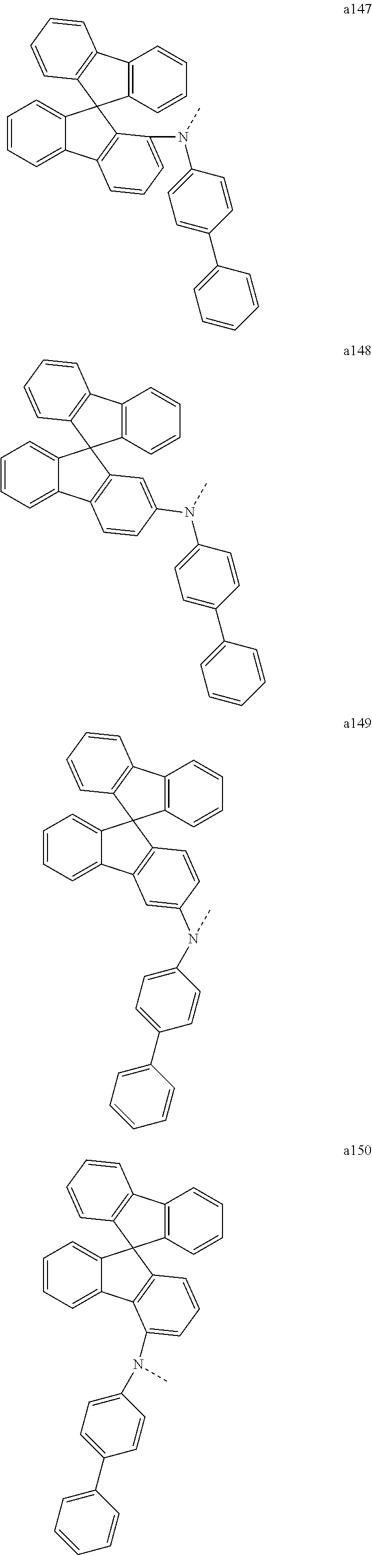 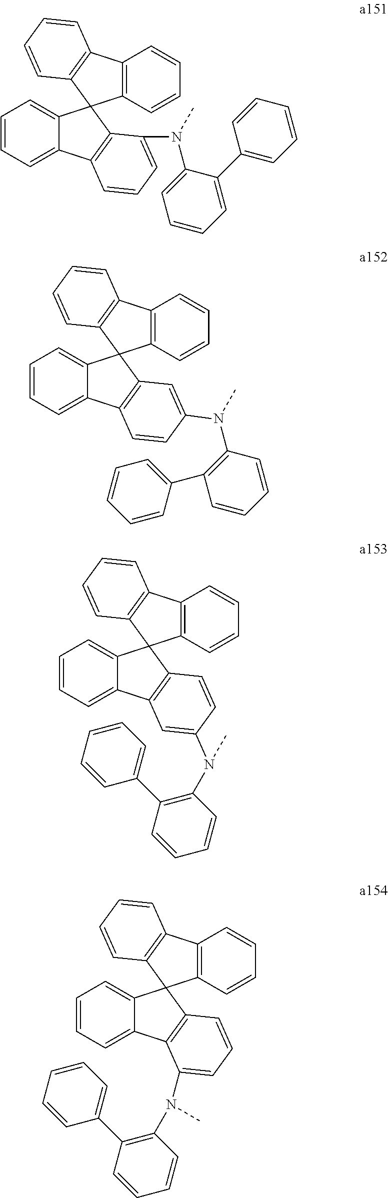 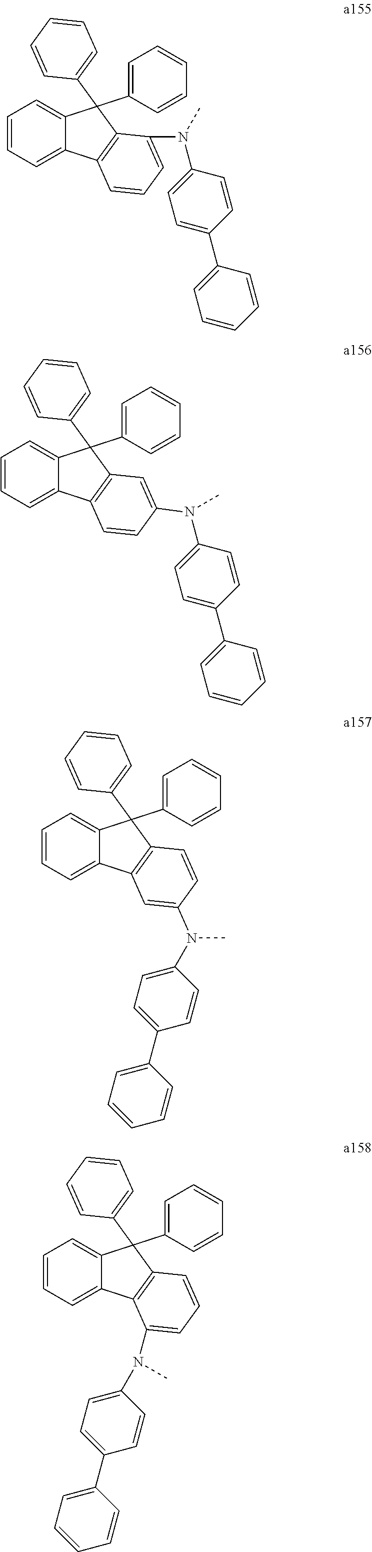 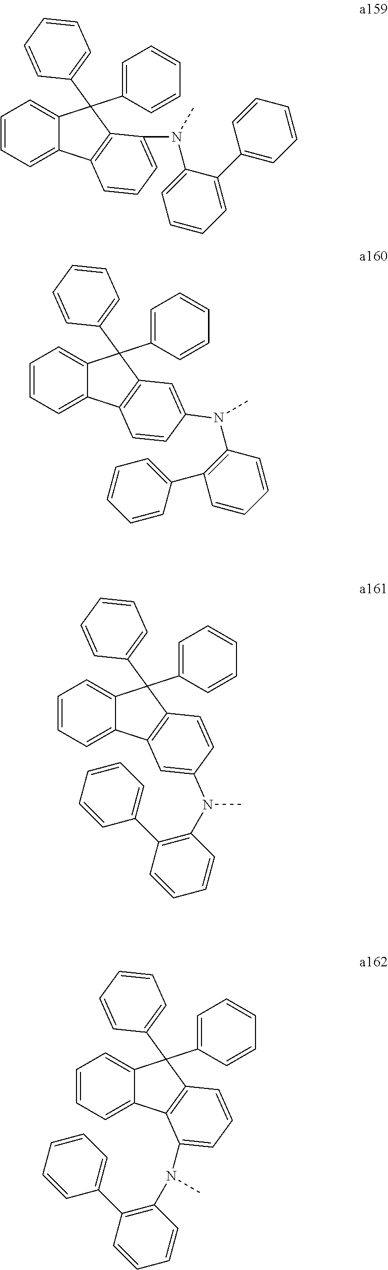 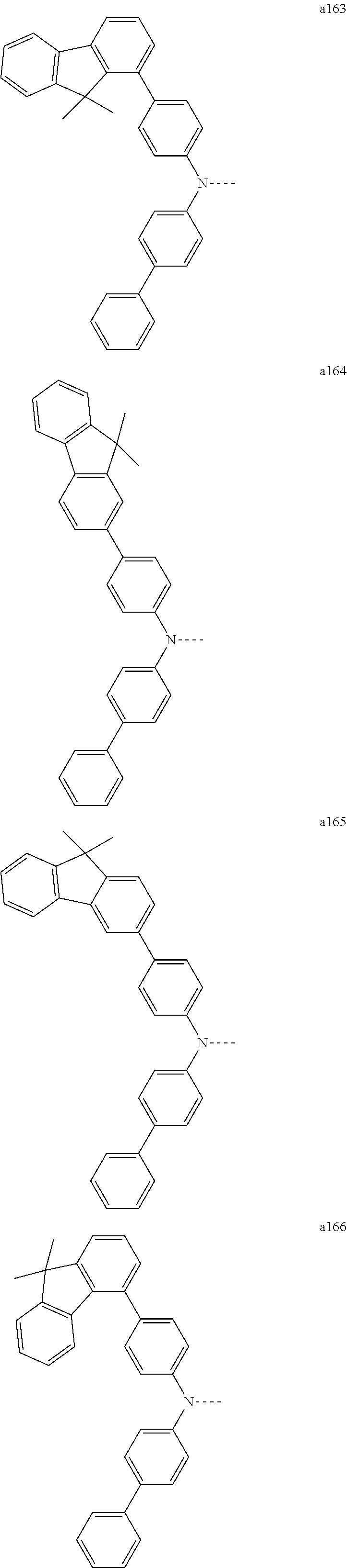 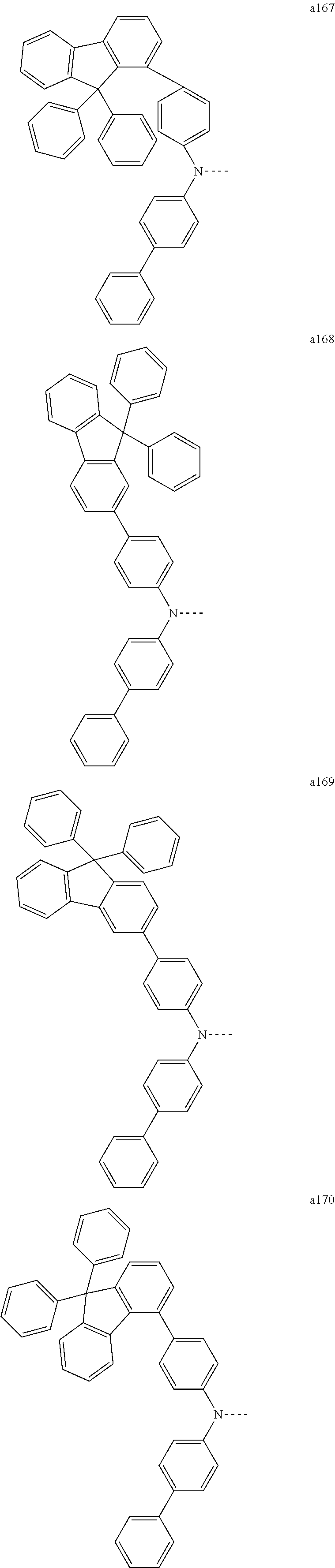 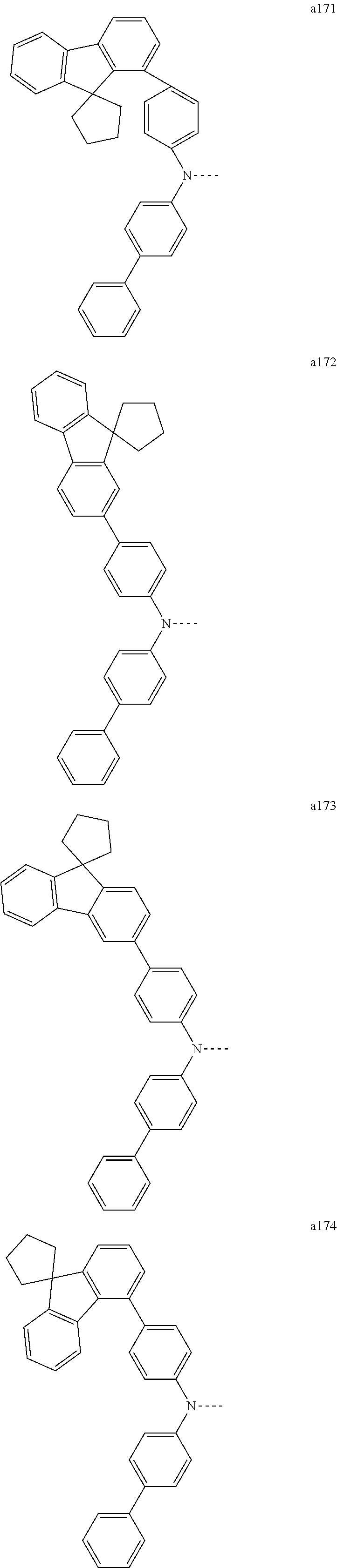 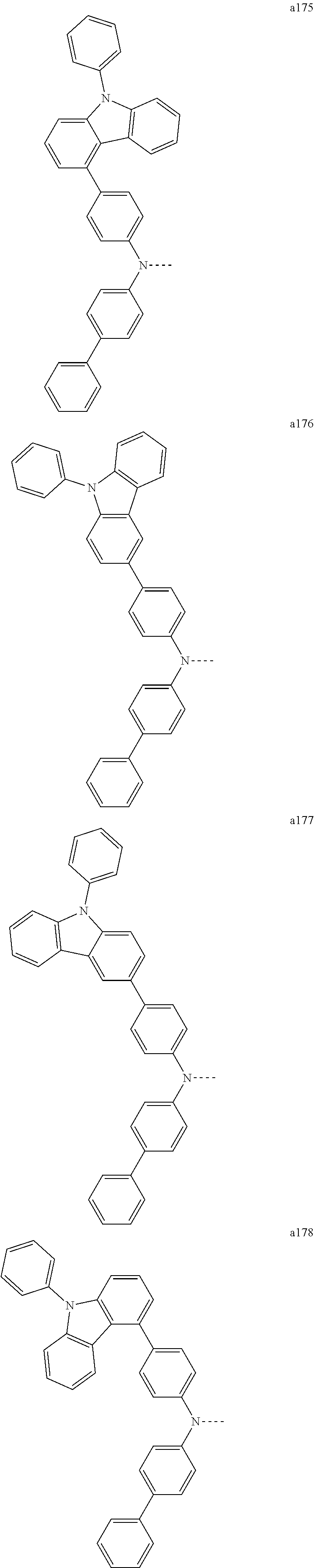 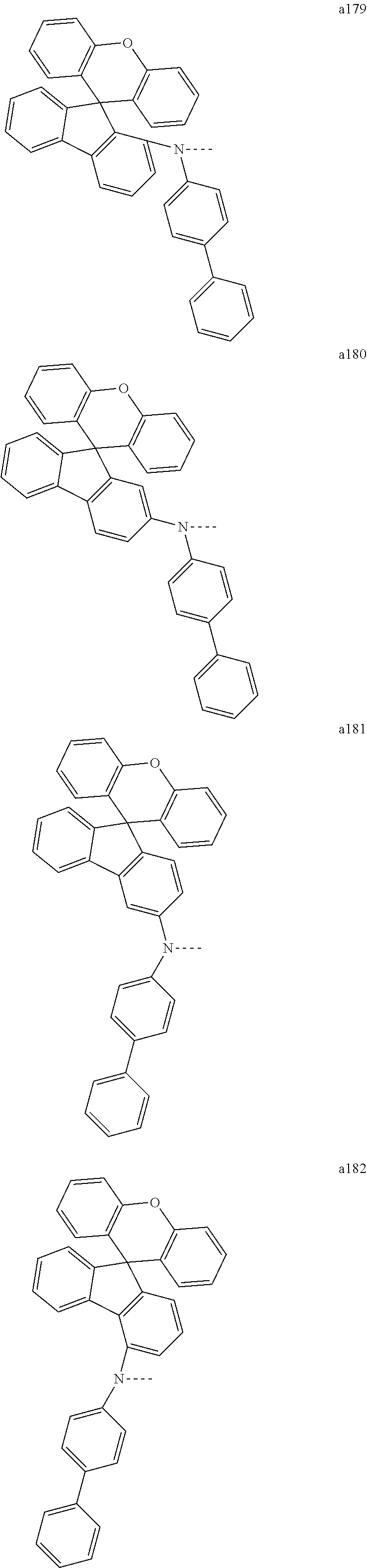 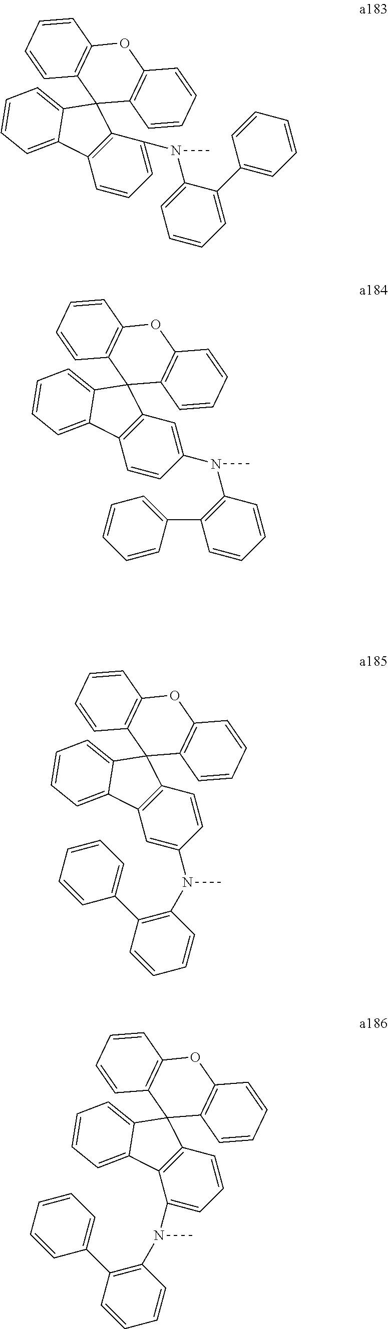 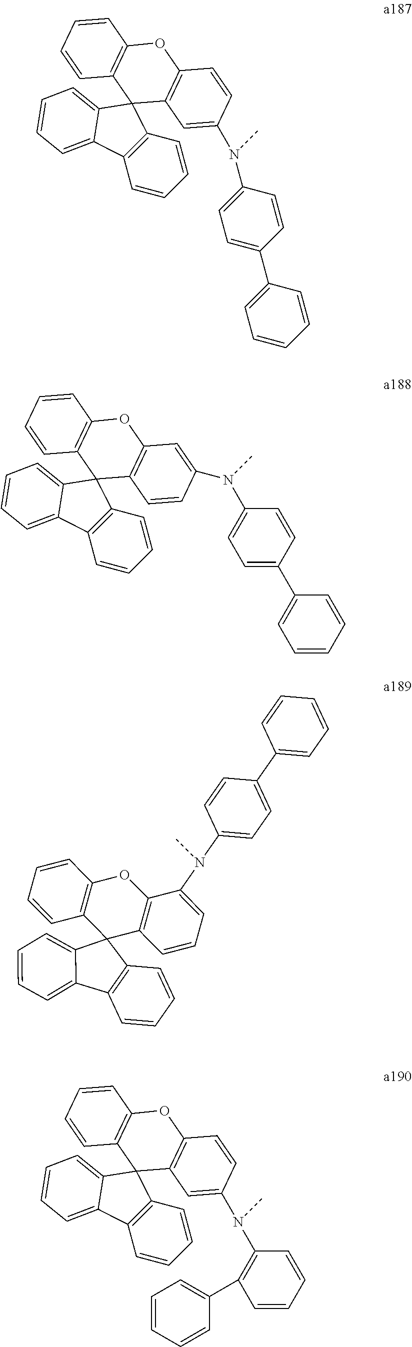 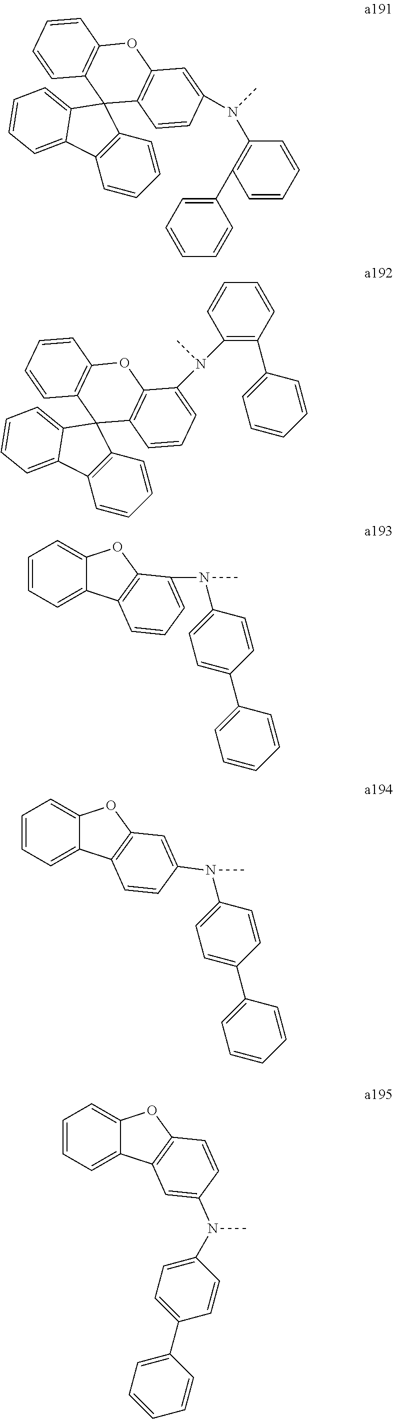 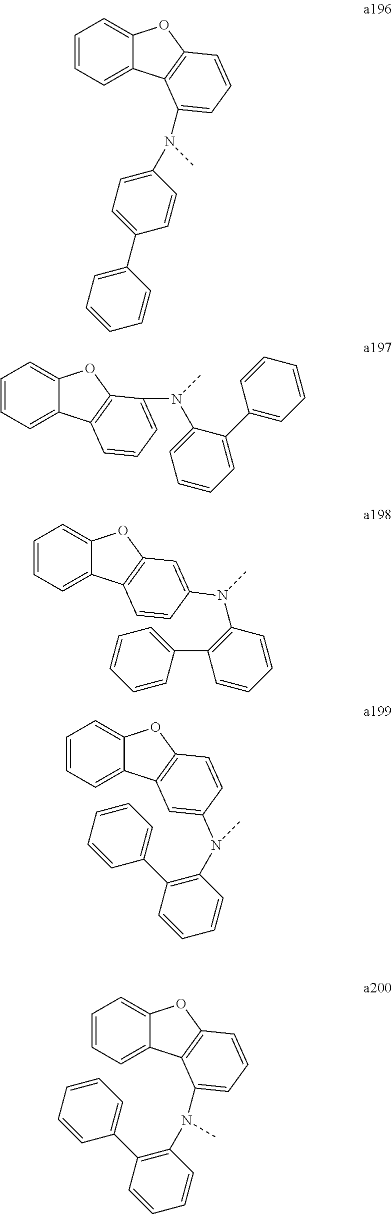 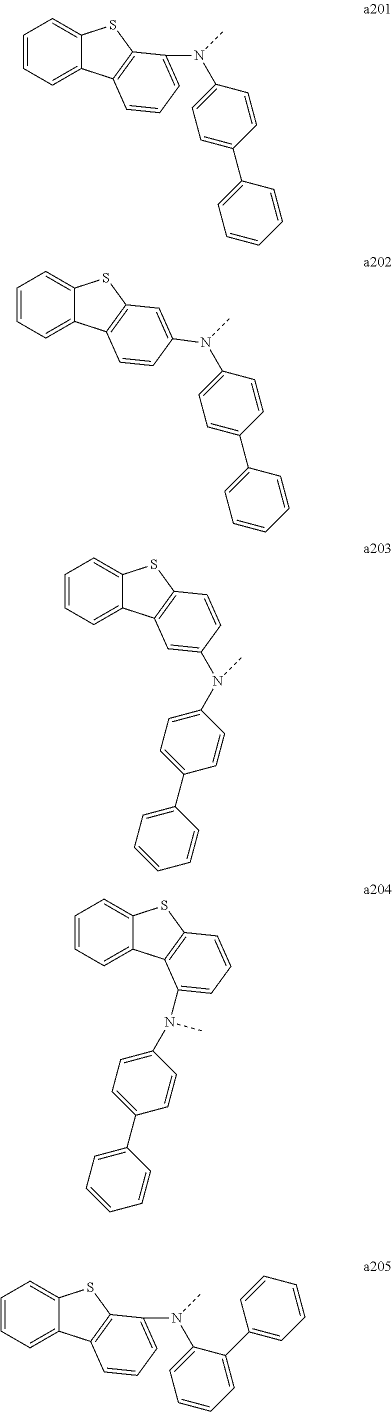 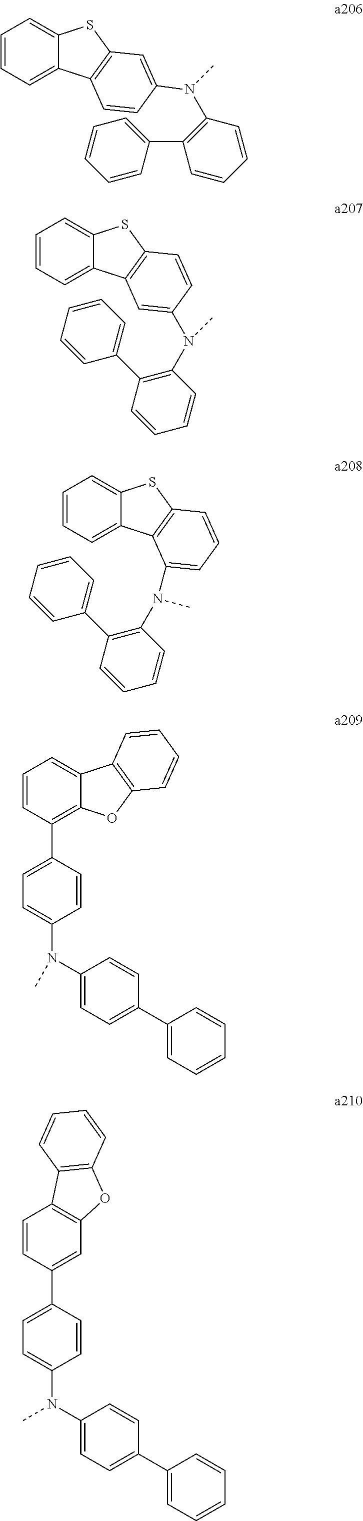 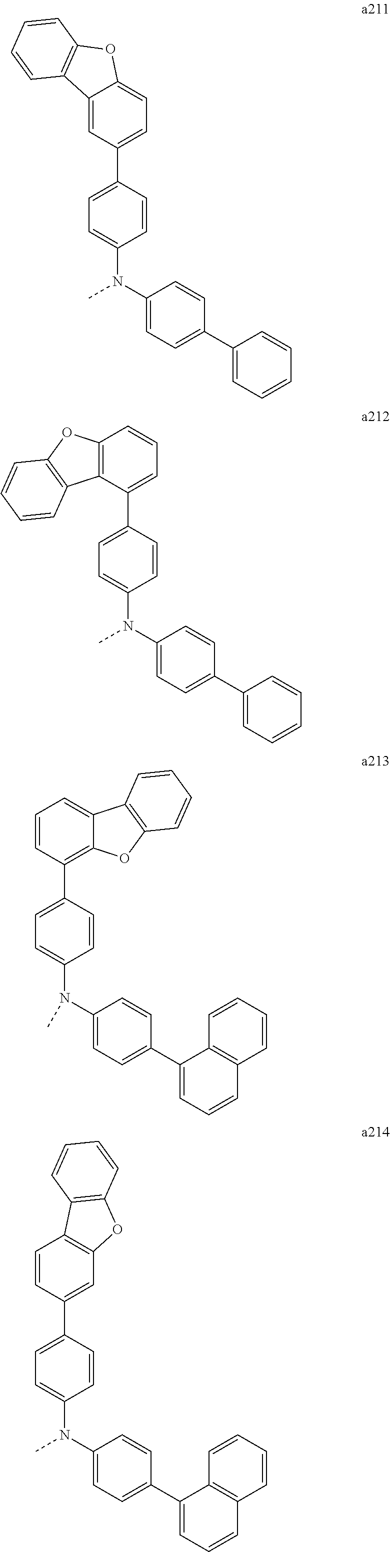 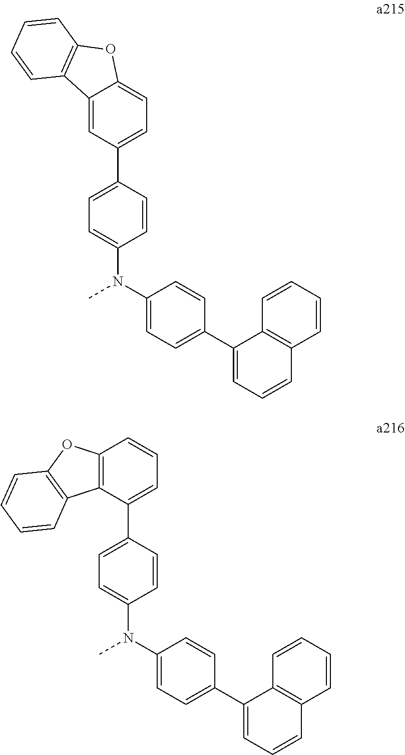 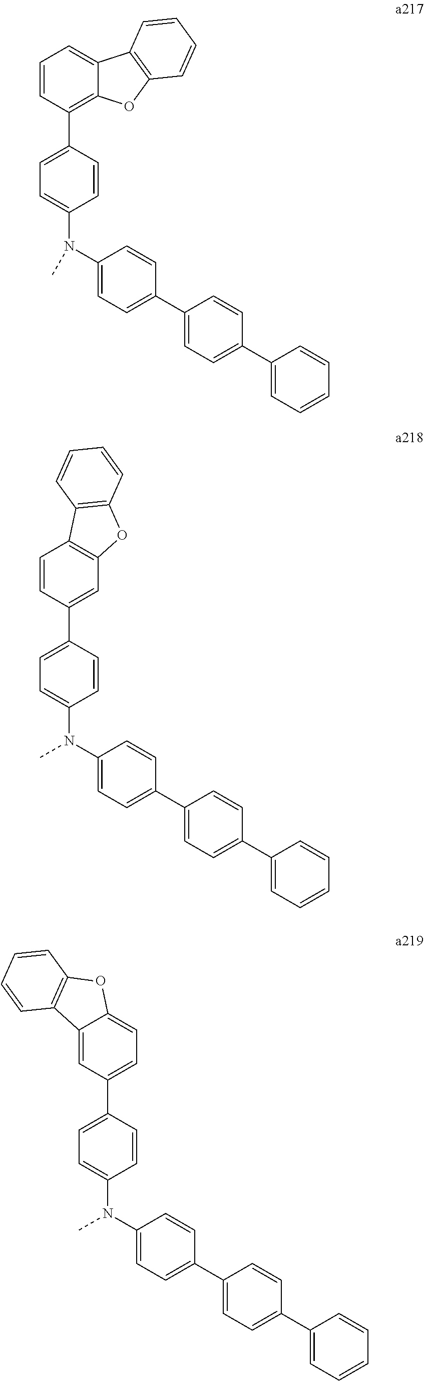 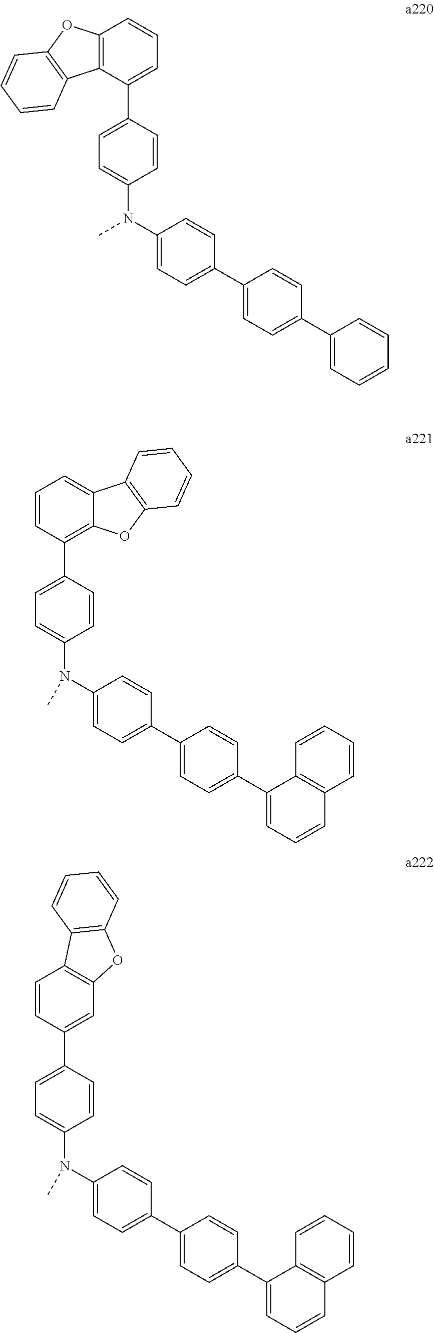 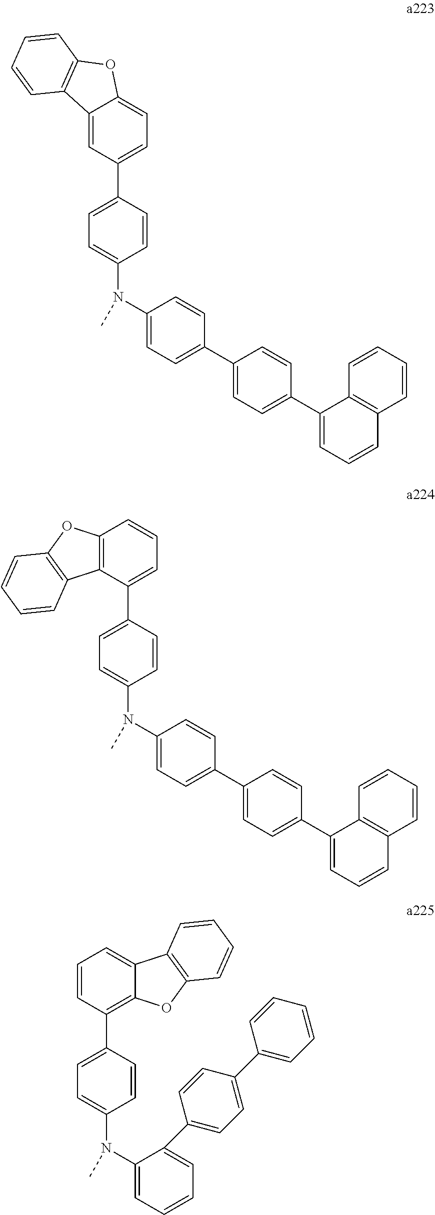 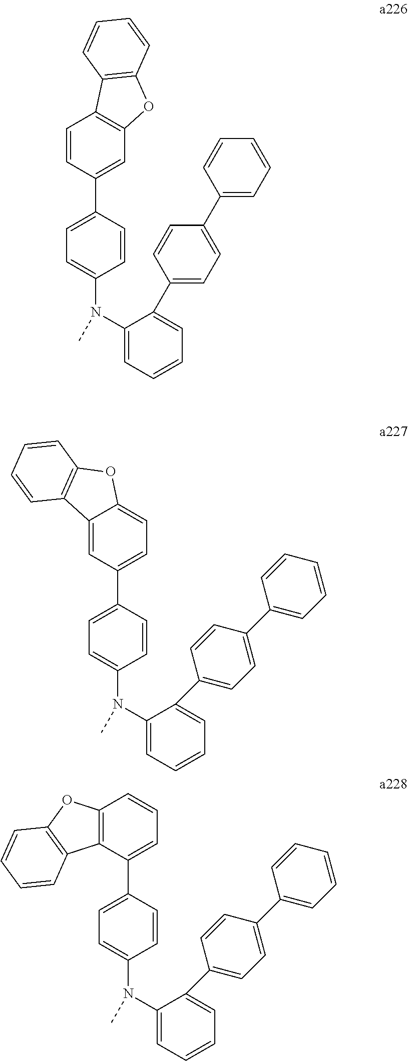 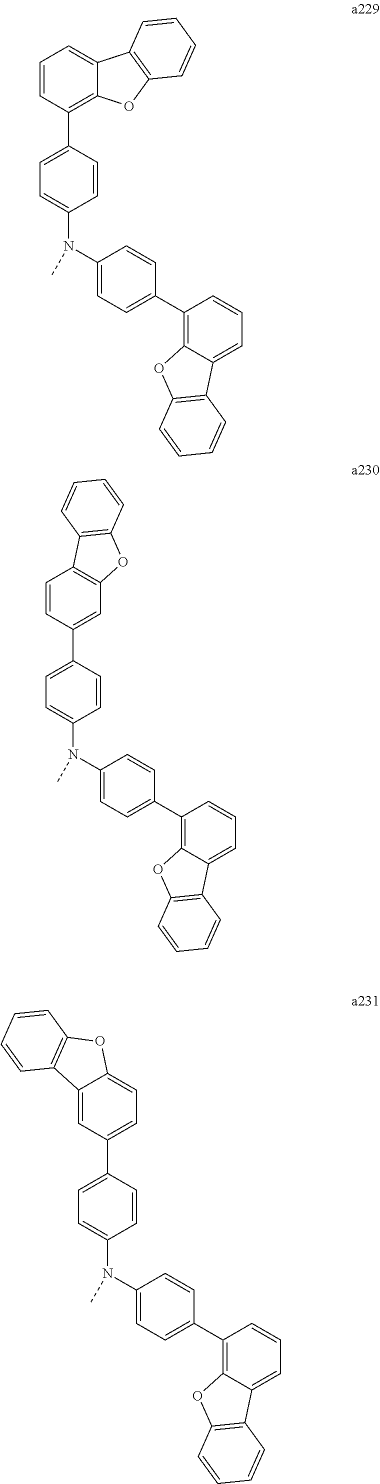 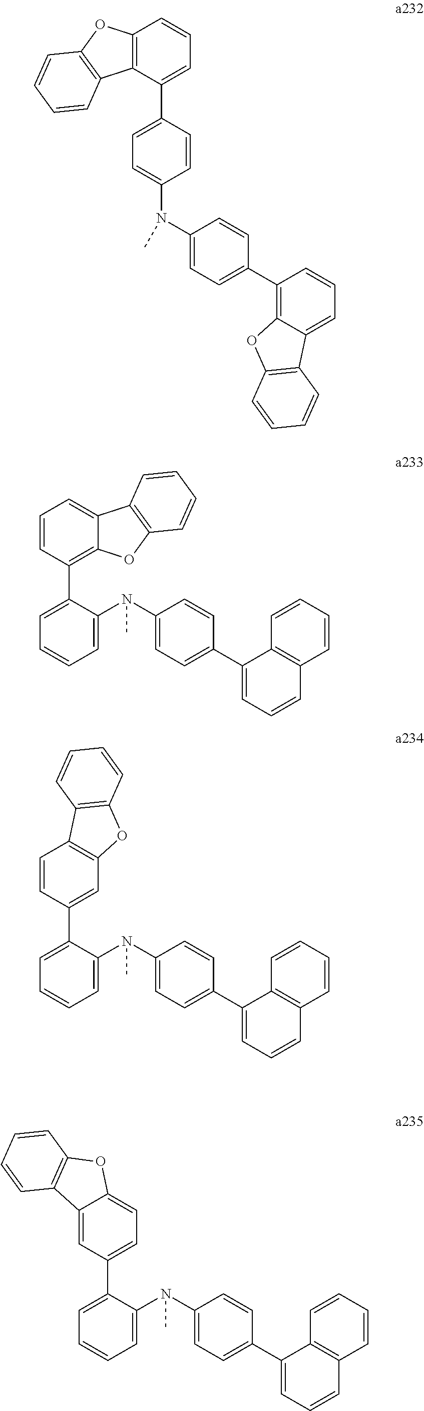 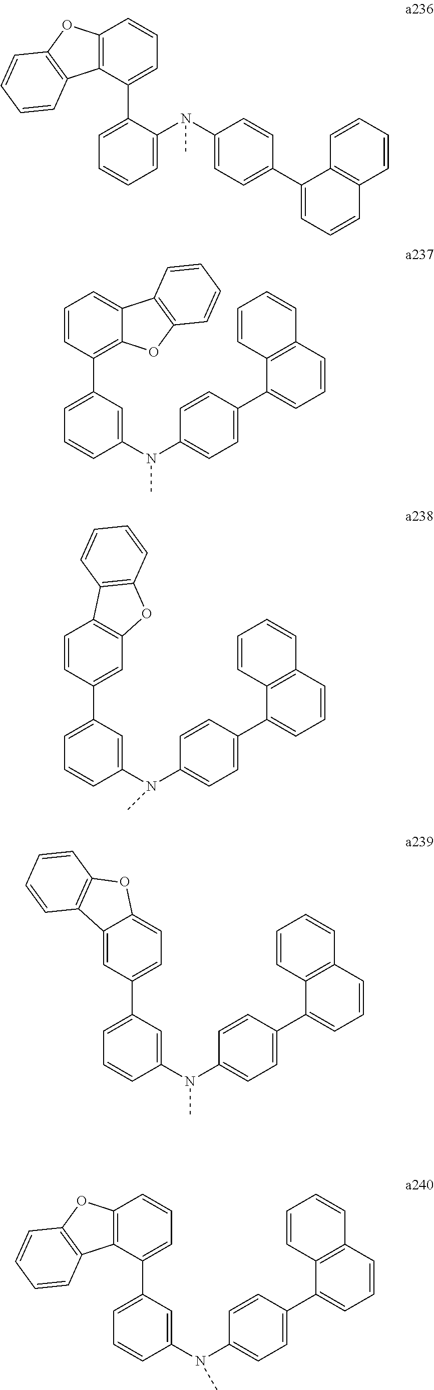 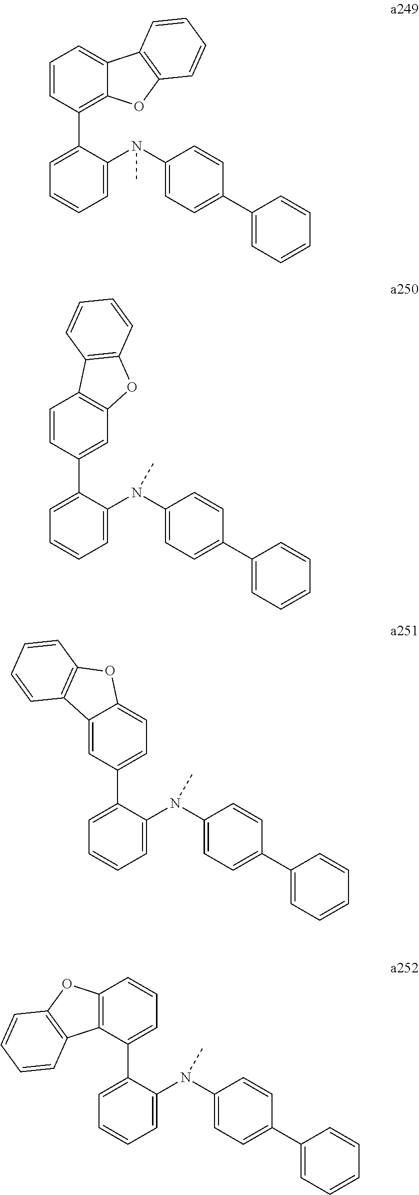 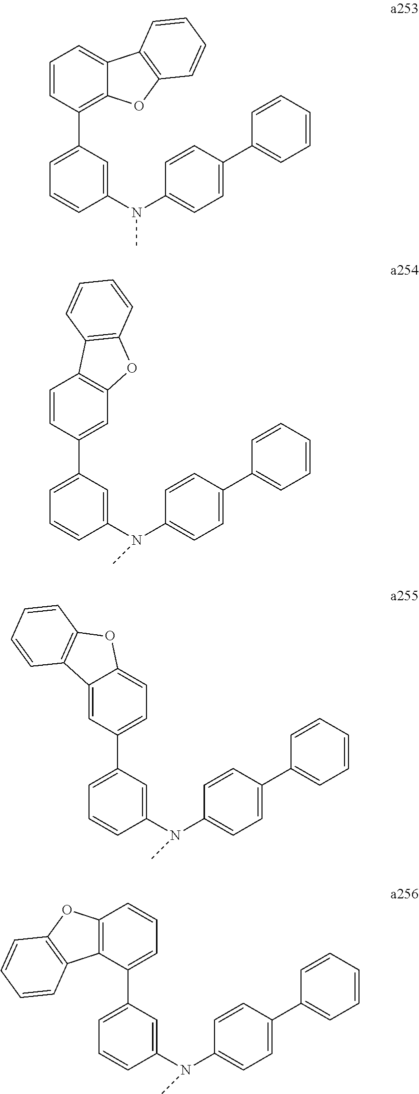 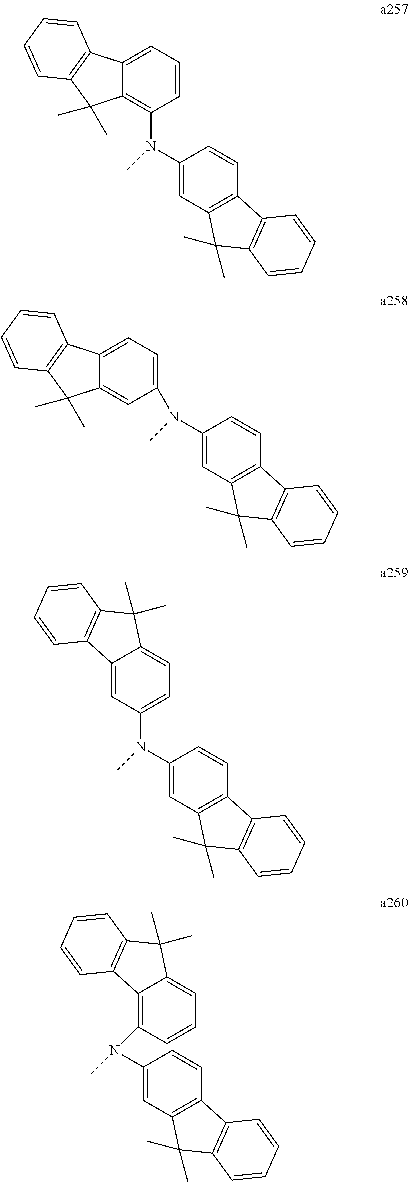 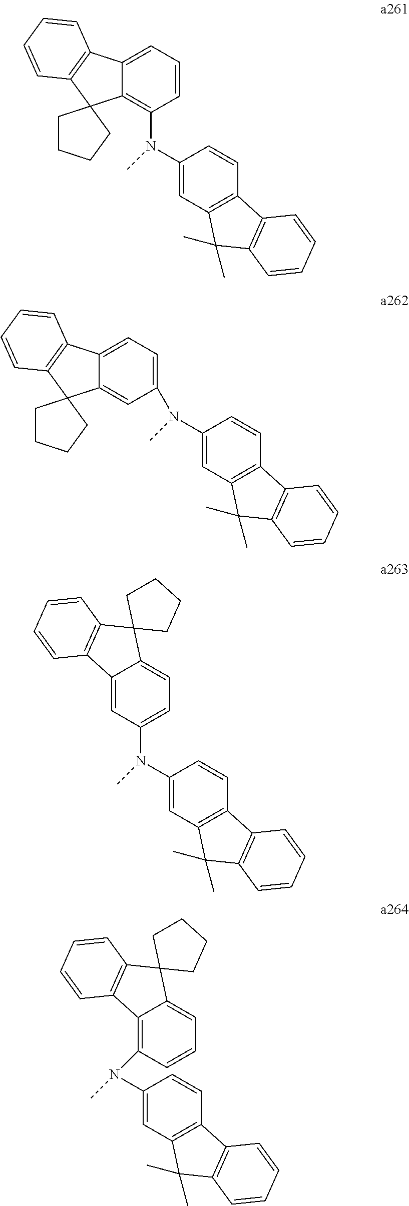 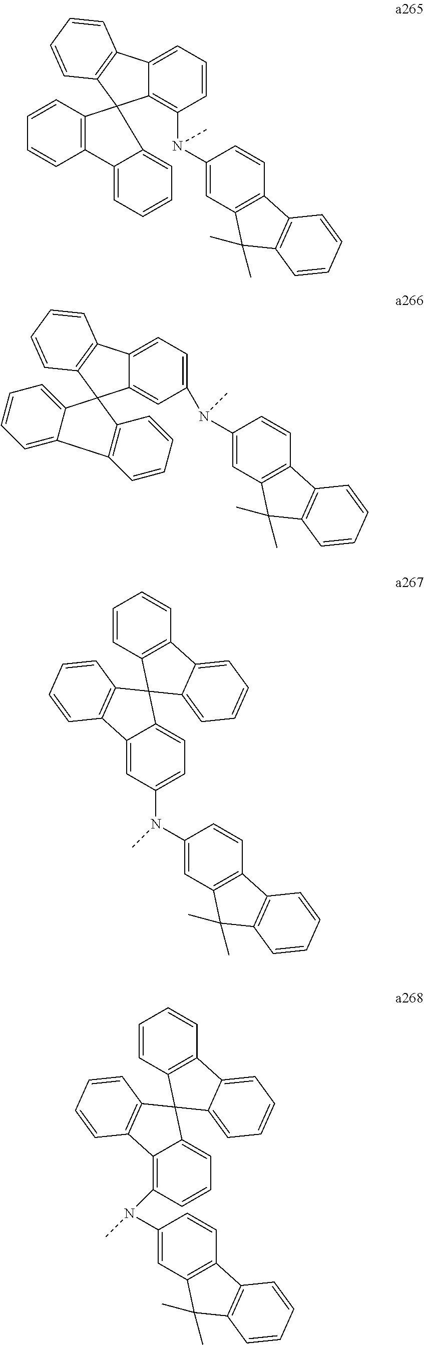 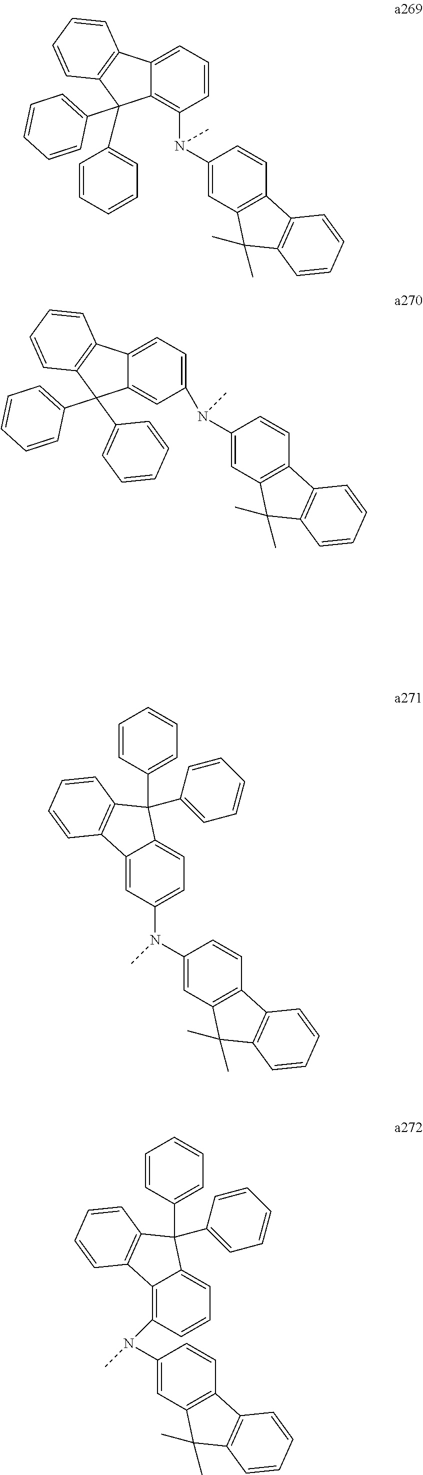 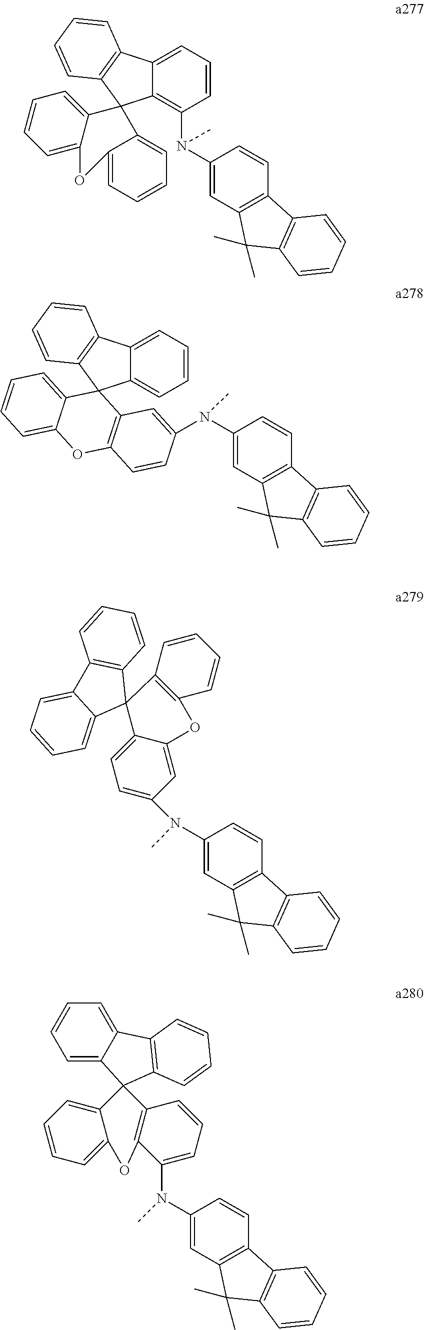 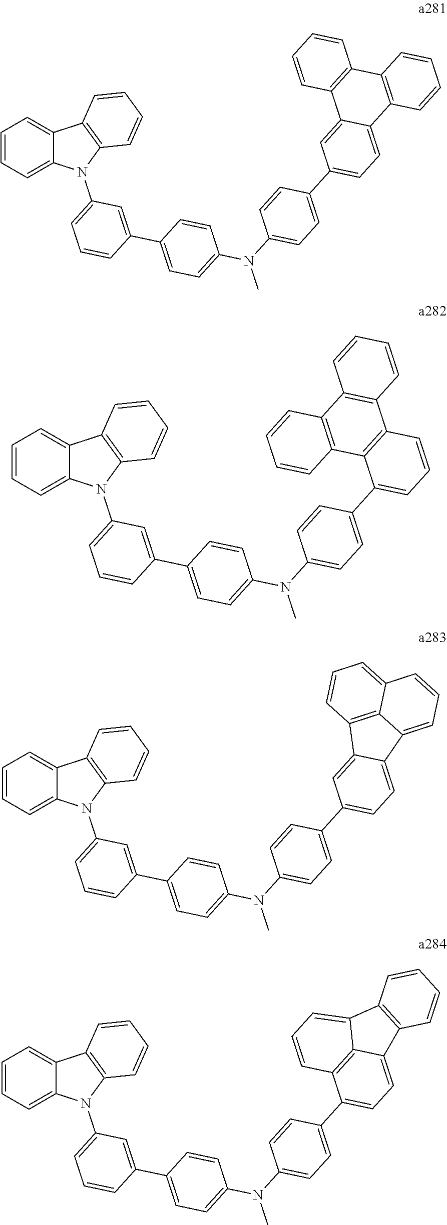 [SMILES: CN(c1ccc(-c2cccc(-n3c4ccccc4c4ccccc43)c2)cc1)c1ccc(-c2ccc3c4ccccc4c4ccccc4c3c2)cc1 ; CN(c1ccc(-c2cccc(-n3c4ccccc4c4ccccc43)c2)cc1)c1ccc(-c2cccc3c4ccccc4c4ccccc4c23)cc1 ; CN(c1ccc(-c2cccc(-n3c4ccccc4c4ccccc43)c2)cc1)c1ccc(-c2ccc3c(c2)-c2cccc4cccc-3c24)cc1 ; CN(c1ccc(-c2cccc(-n3c4ccccc4c4ccccc43)c2)cc1)c1ccc(-c2ccc3c4c(cccc24)-c2ccccc2-3)cc1] 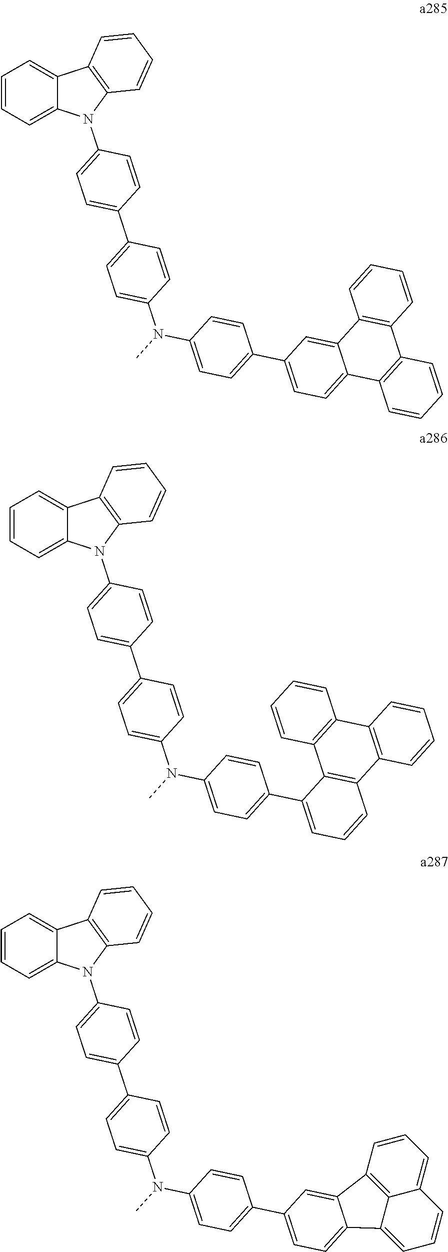 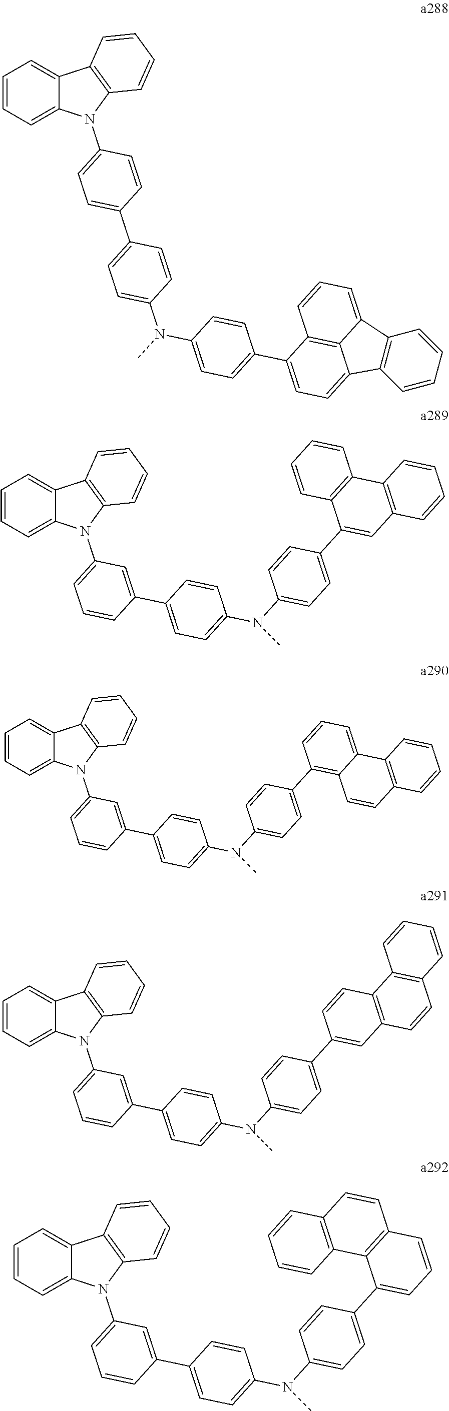 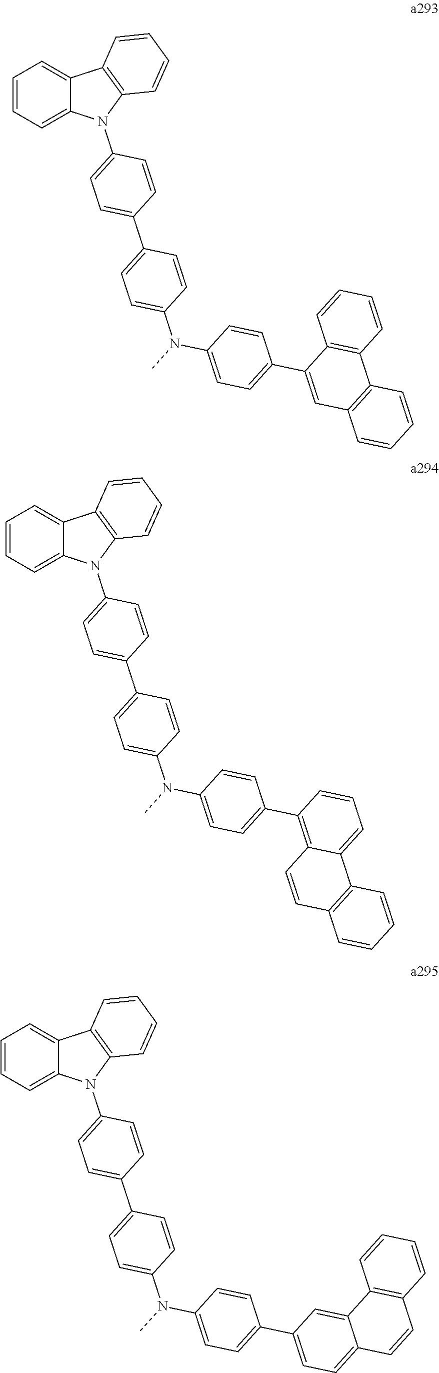 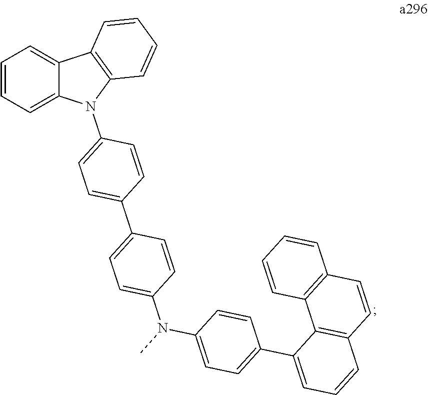 [symbol]
represents a chemical bond;
wherein, the substituted group W3 is formed by replacing one or more hydrogens on the unsubstituted group W3 with deuterium.

14. The aromatic amine compound according to claim 8, wherein the aromatic amine compound is selected from substituted or unsubstituted compound W4, and the unsubstituted compound W4 is selected from the group consisting of the following compounds: 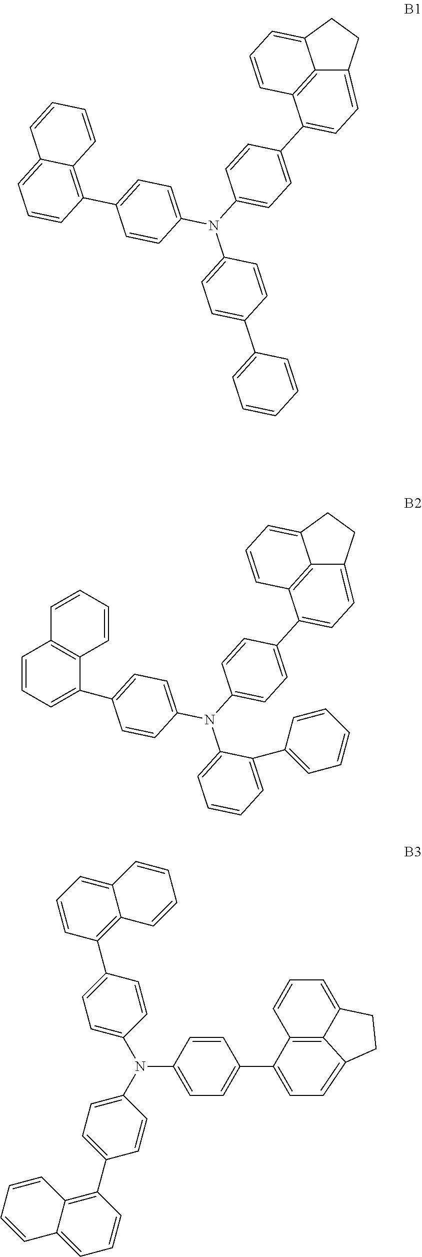 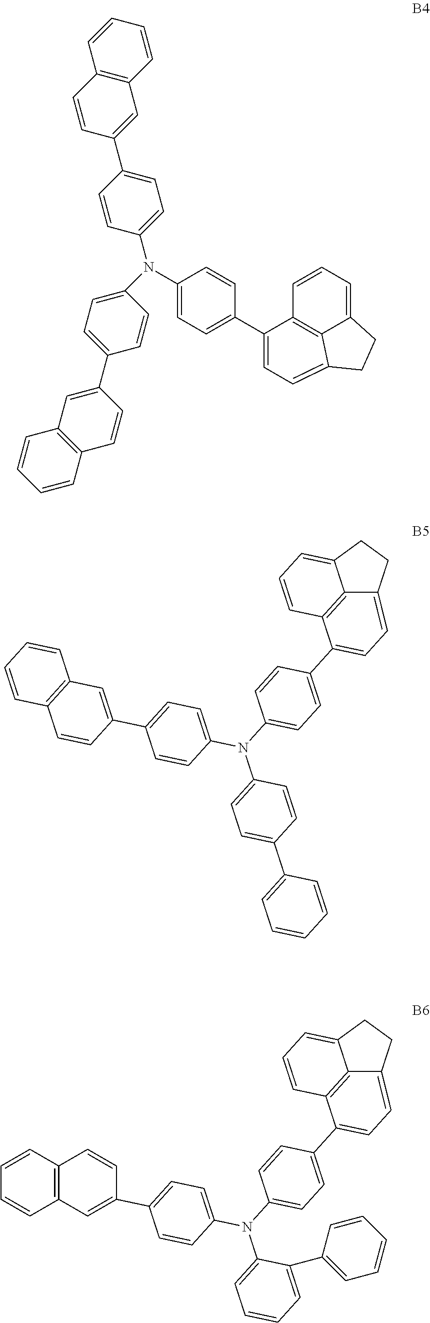 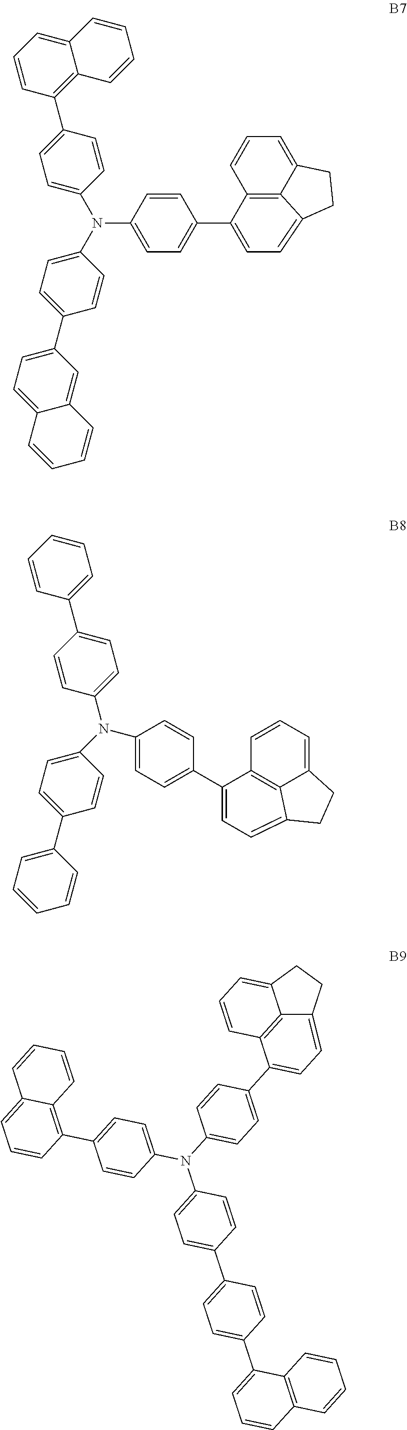 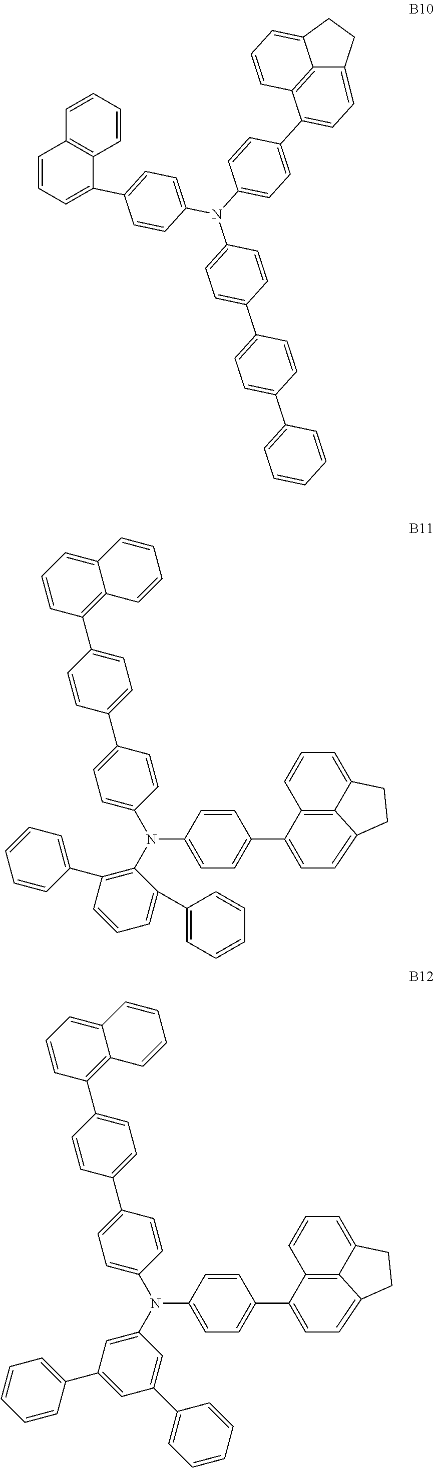 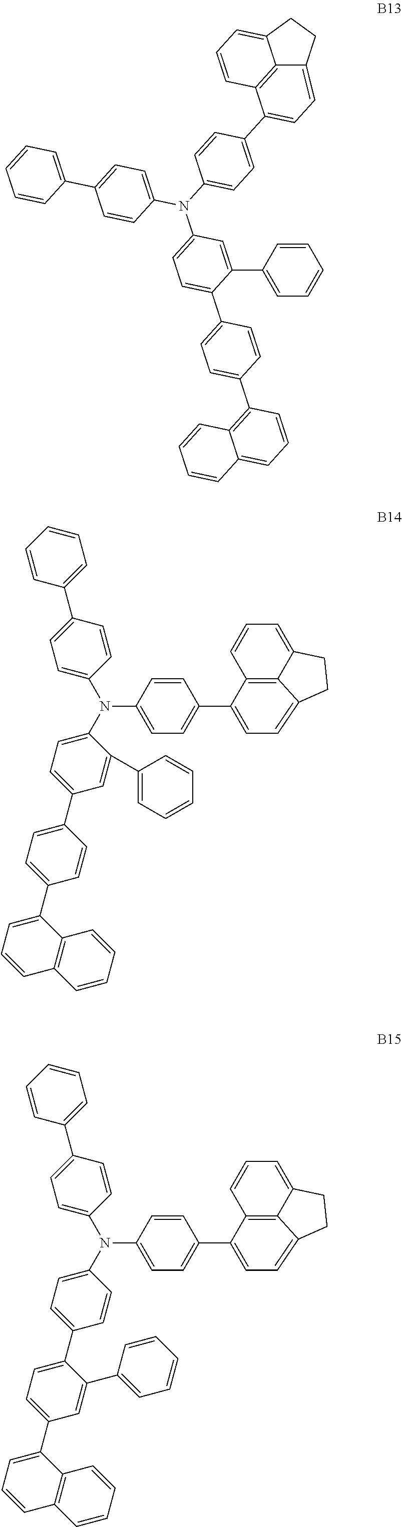 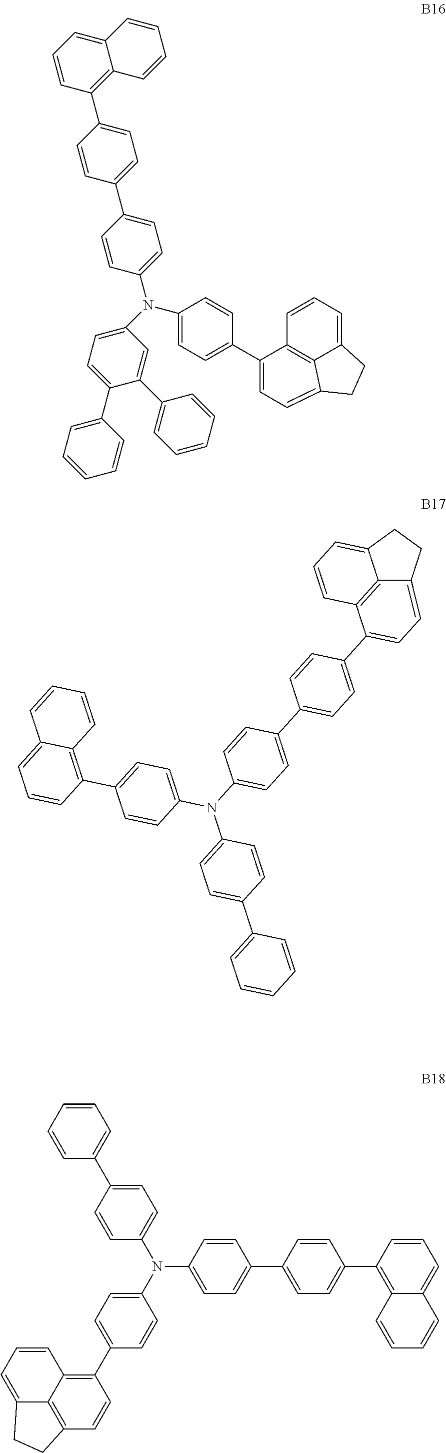 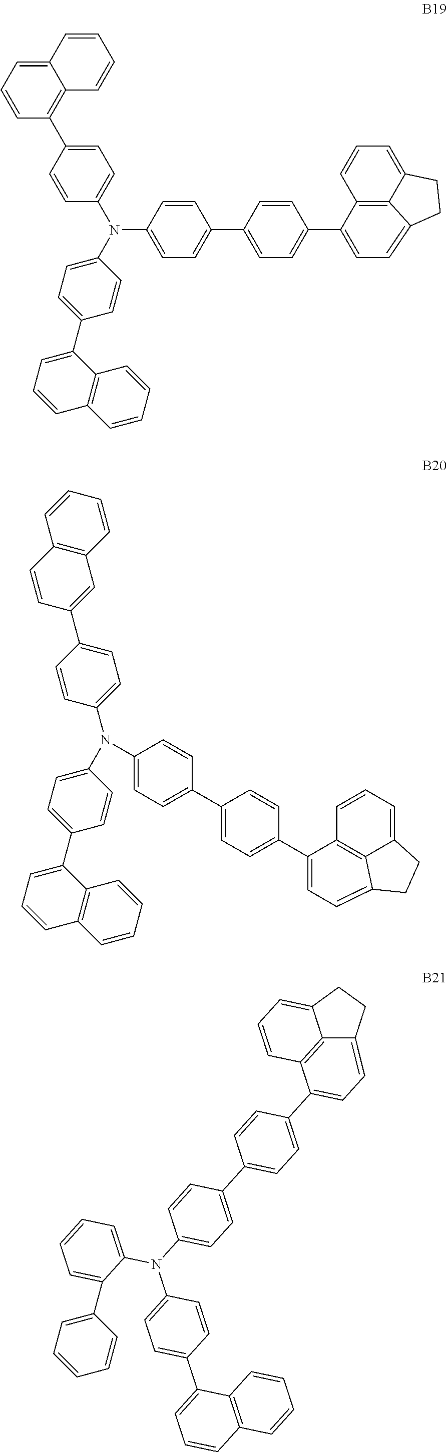 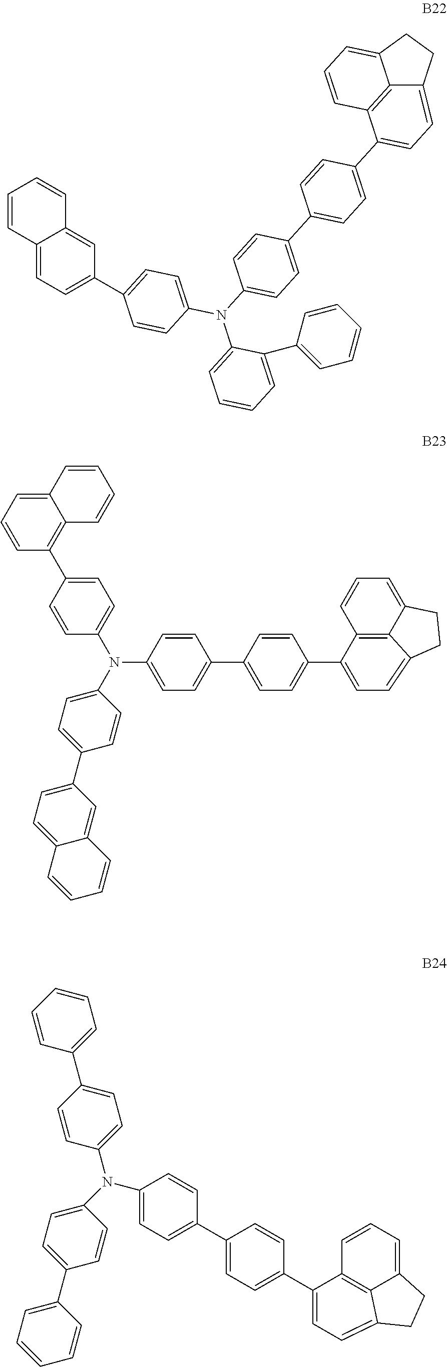 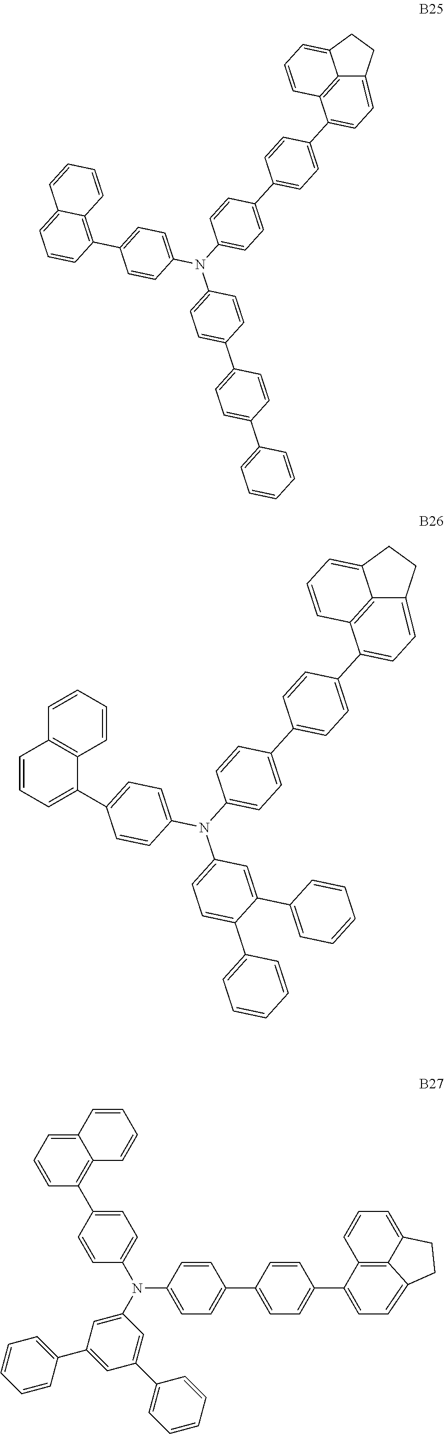 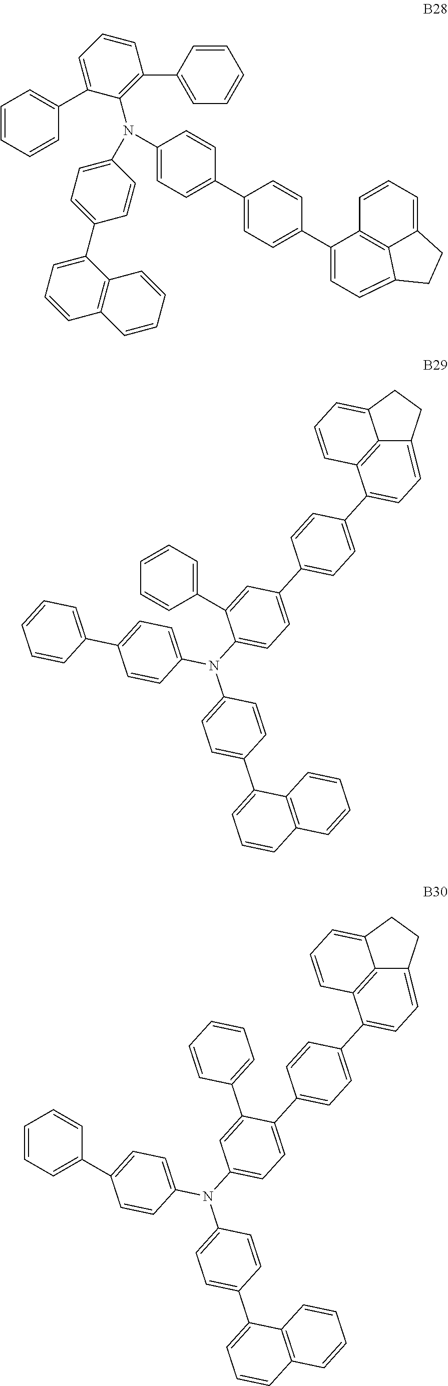 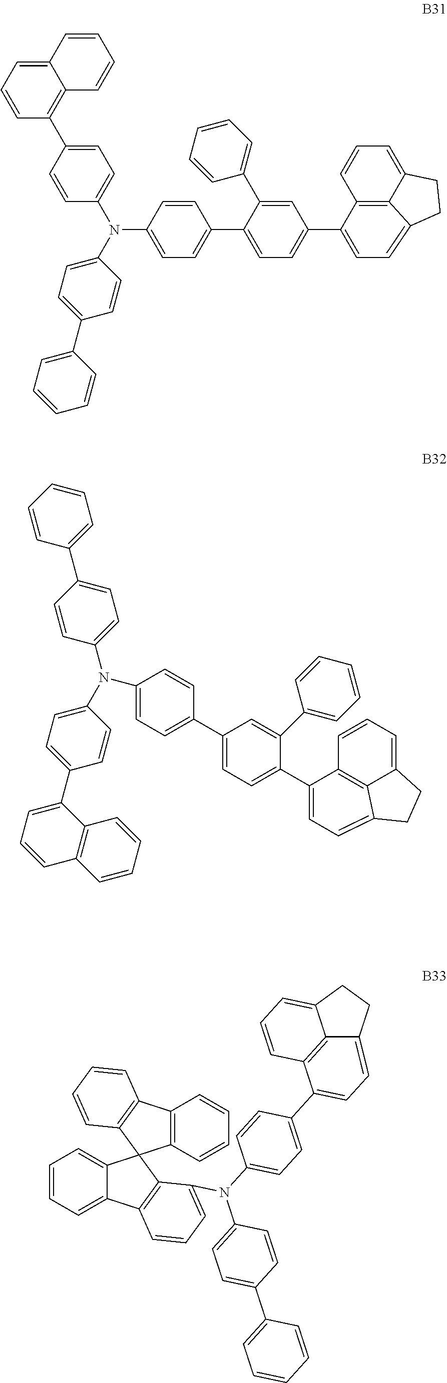 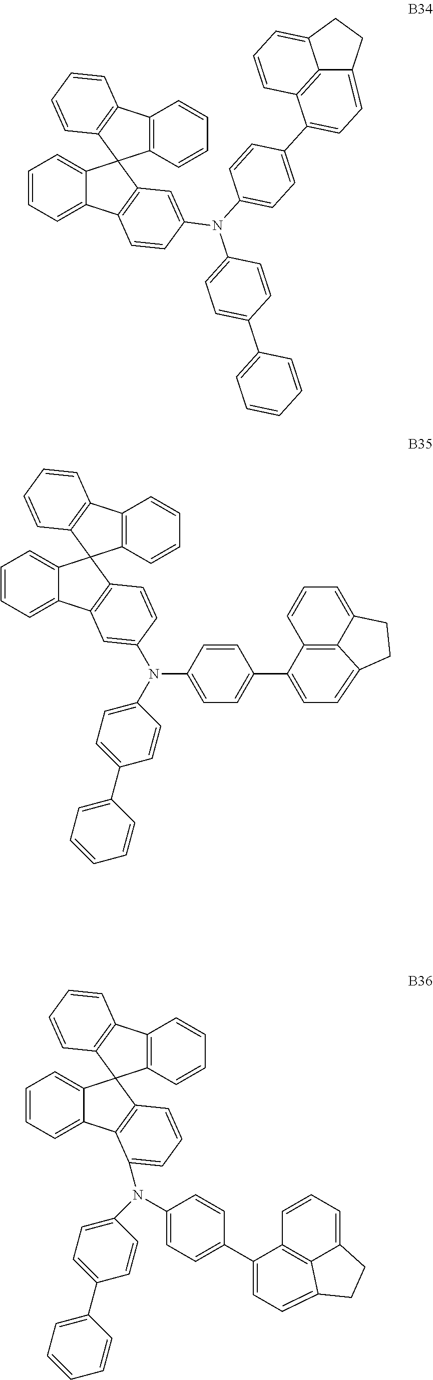 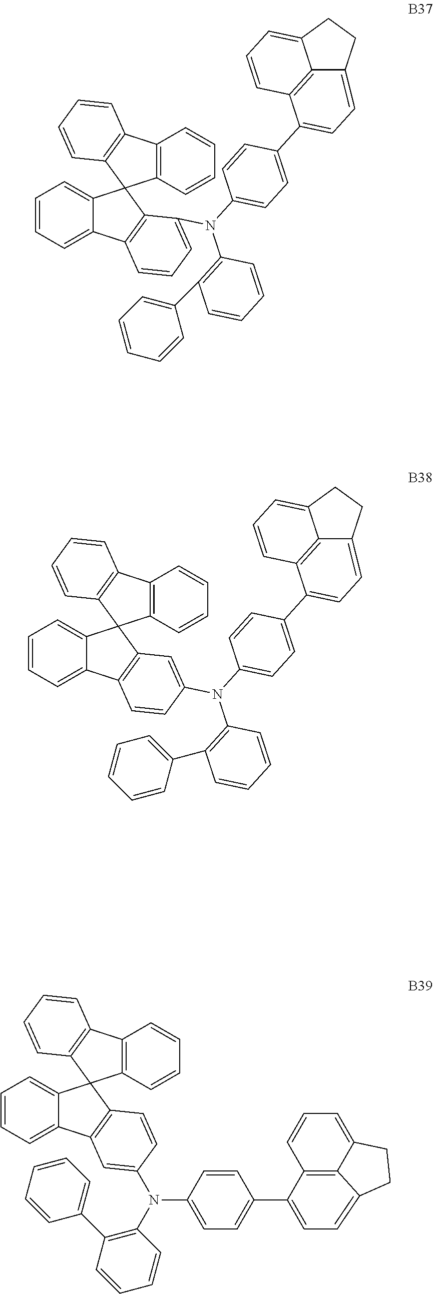 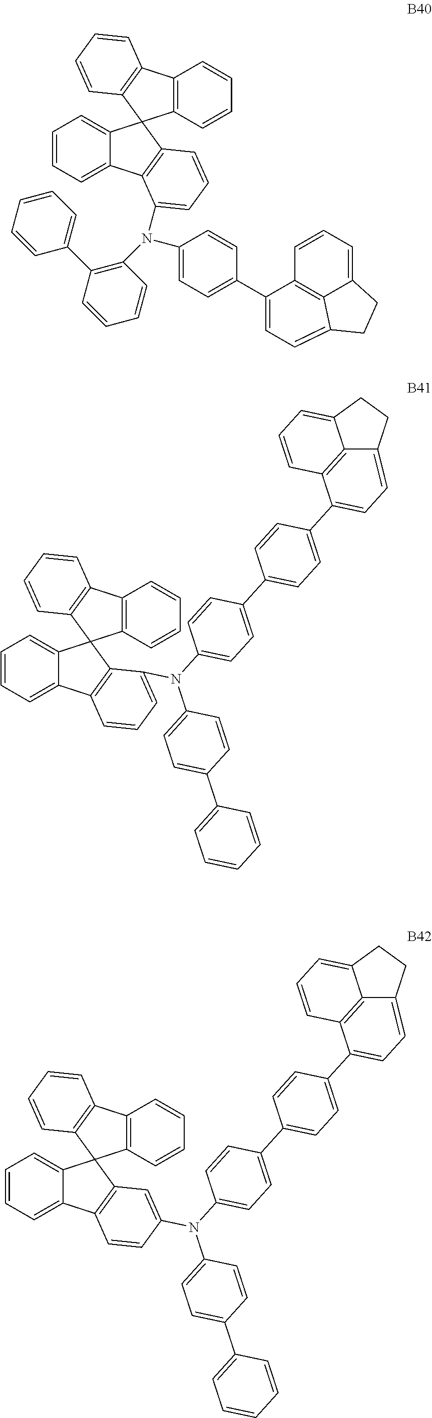 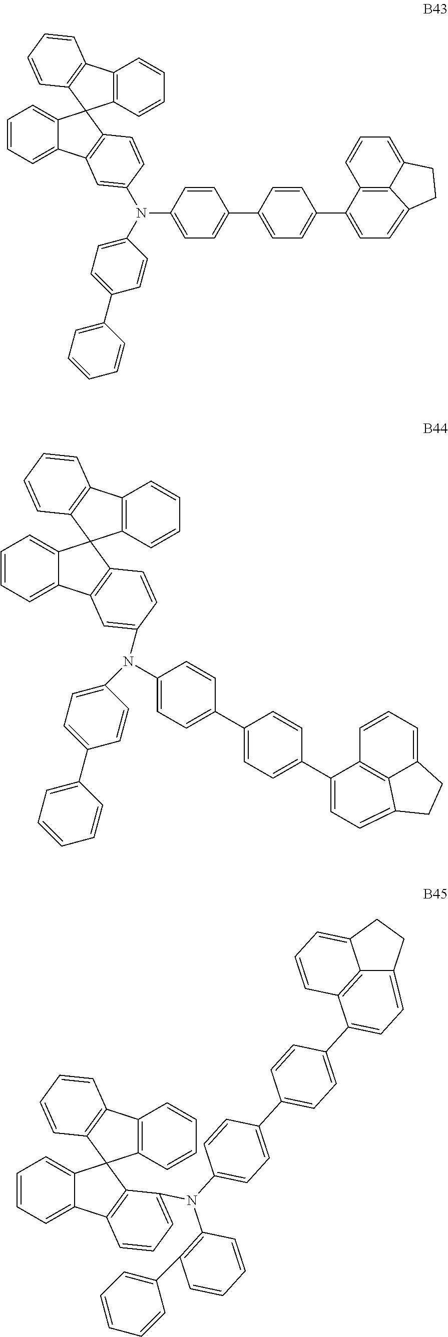 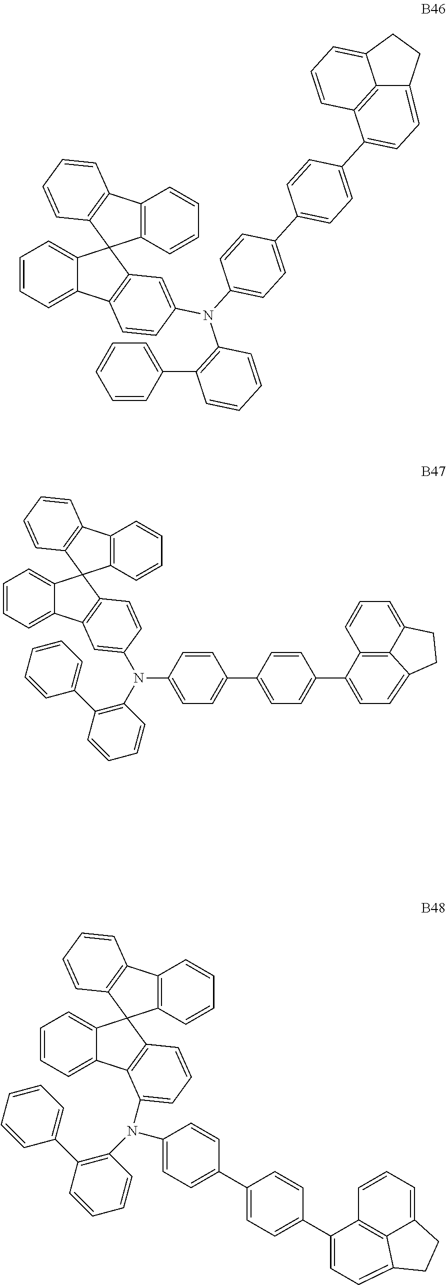 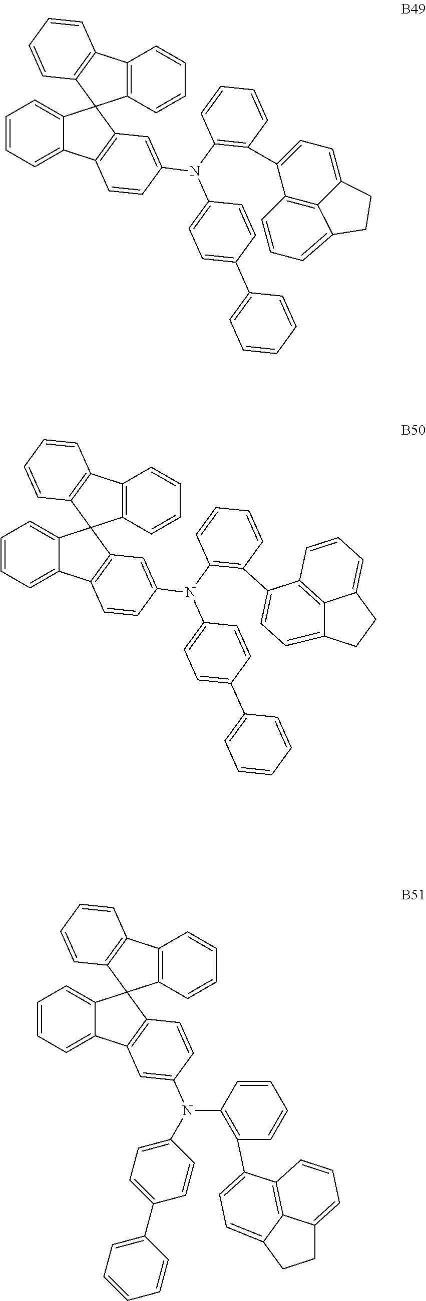 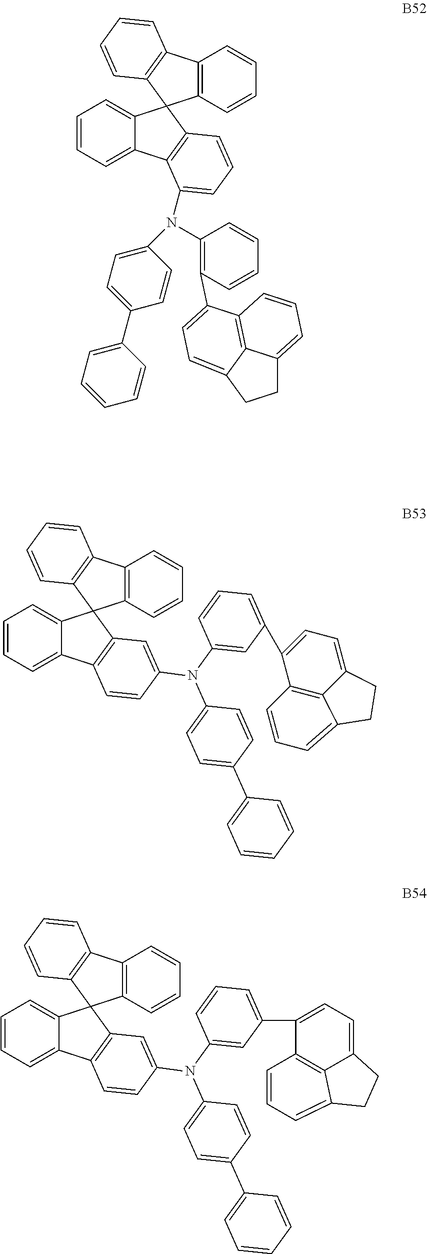 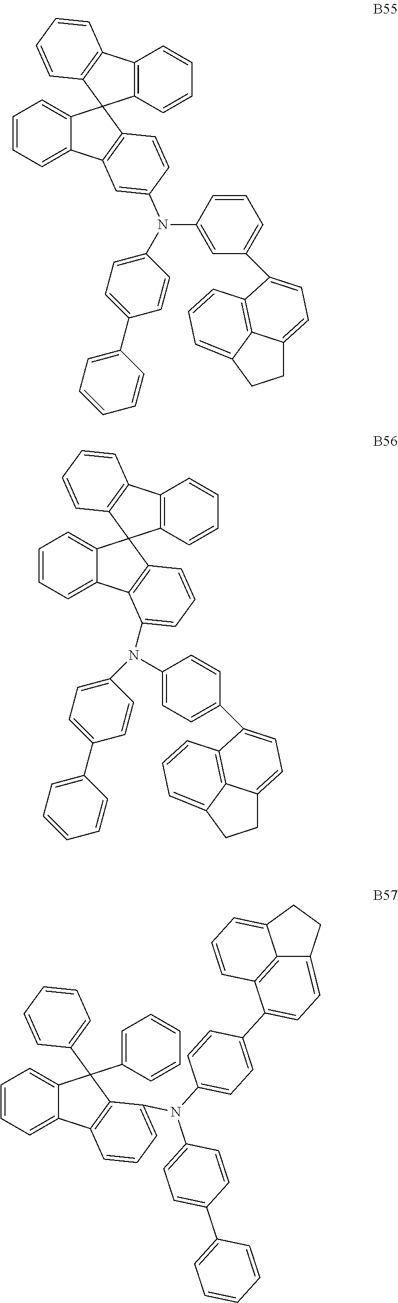 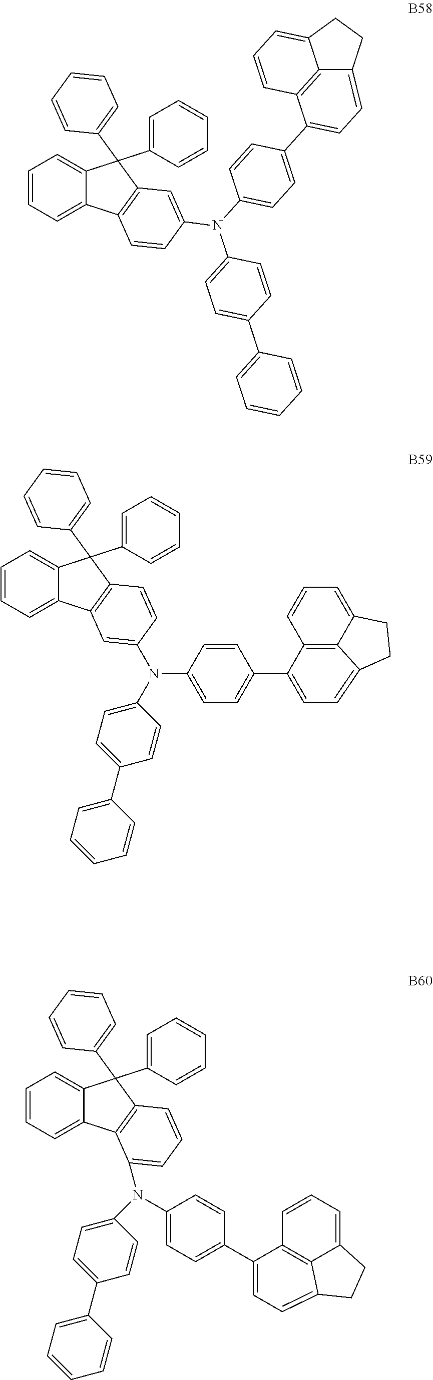 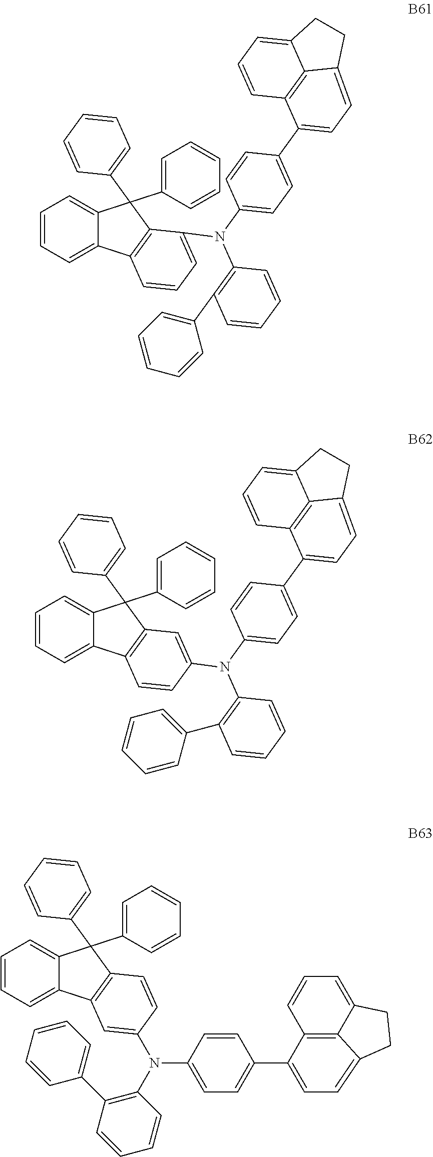 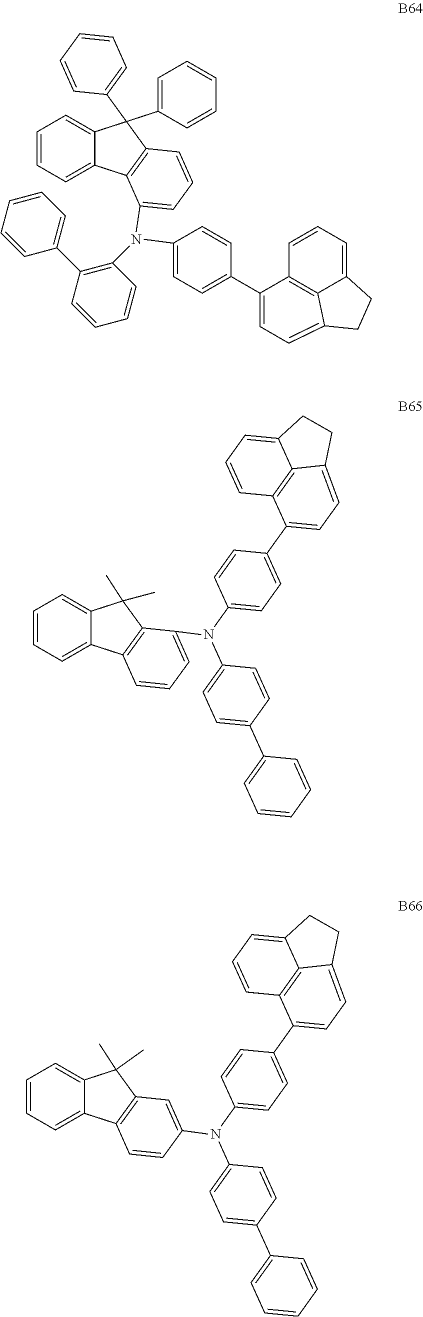 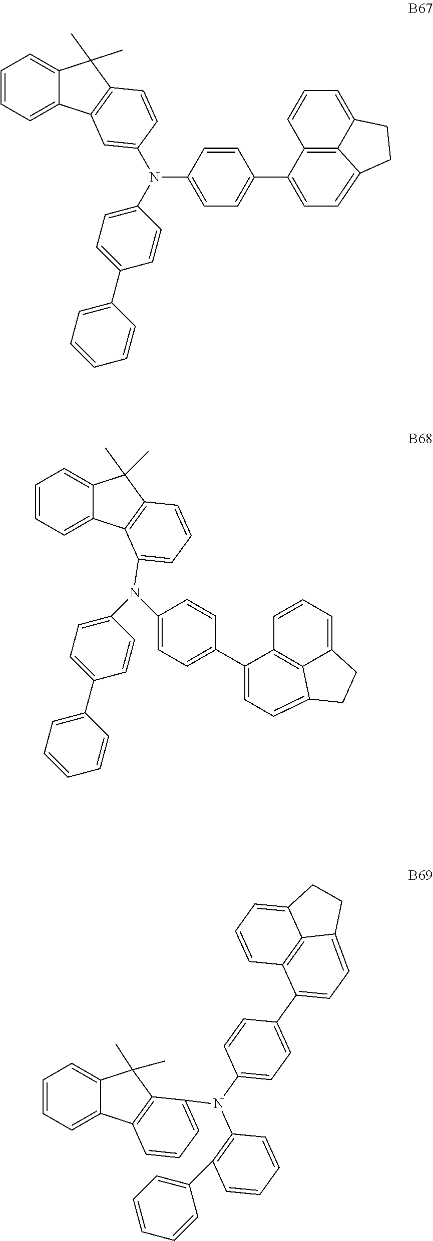 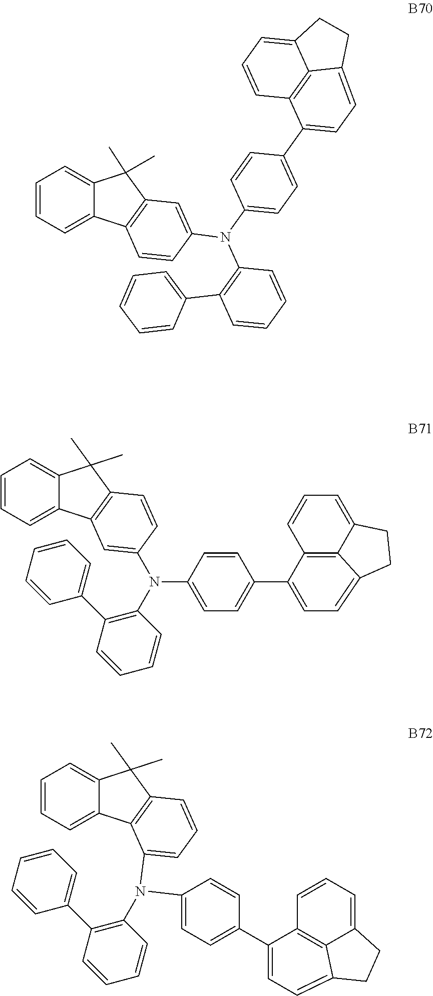 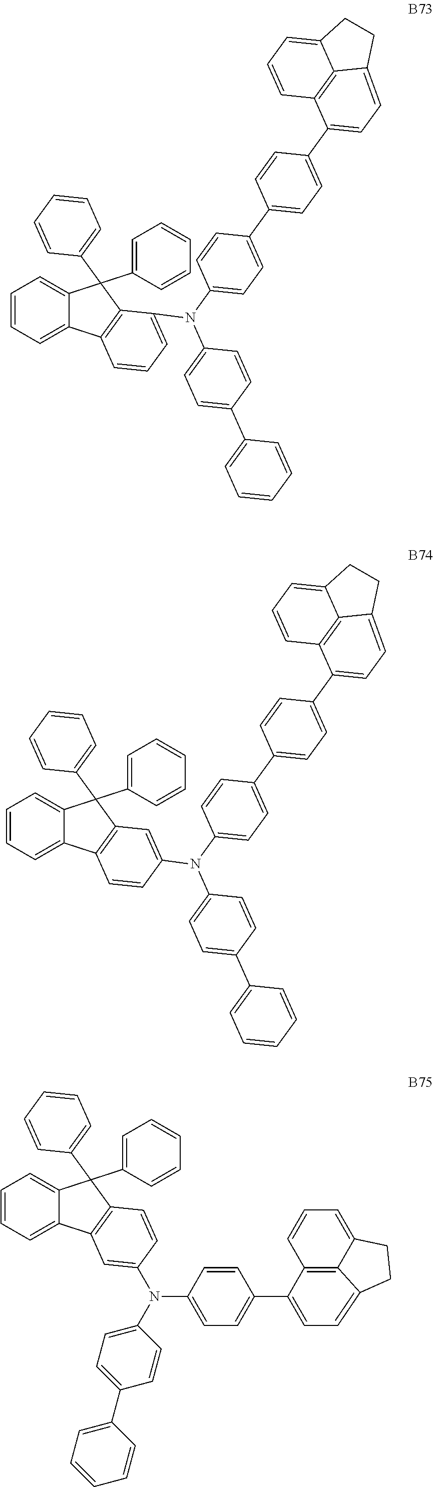 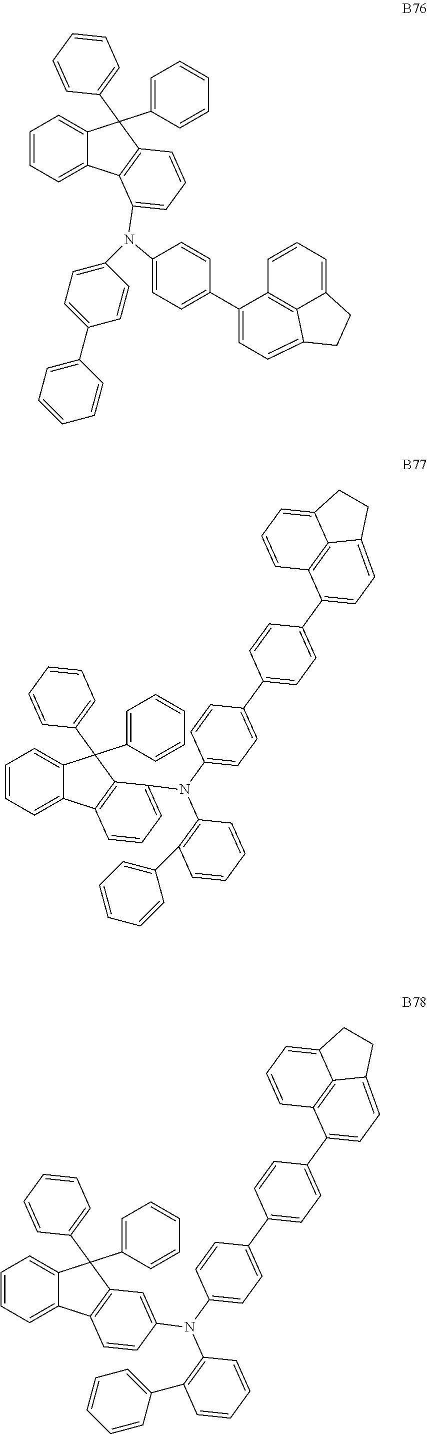 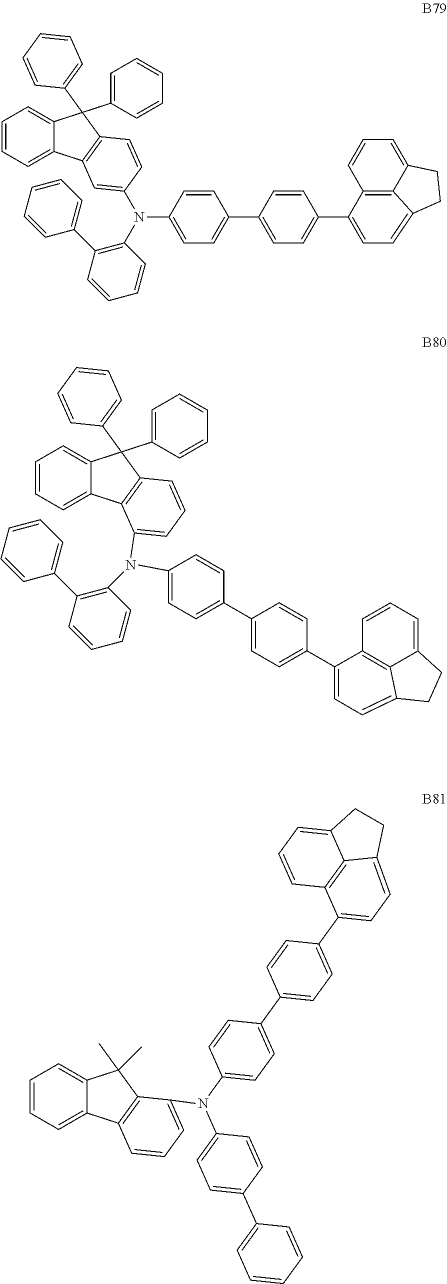 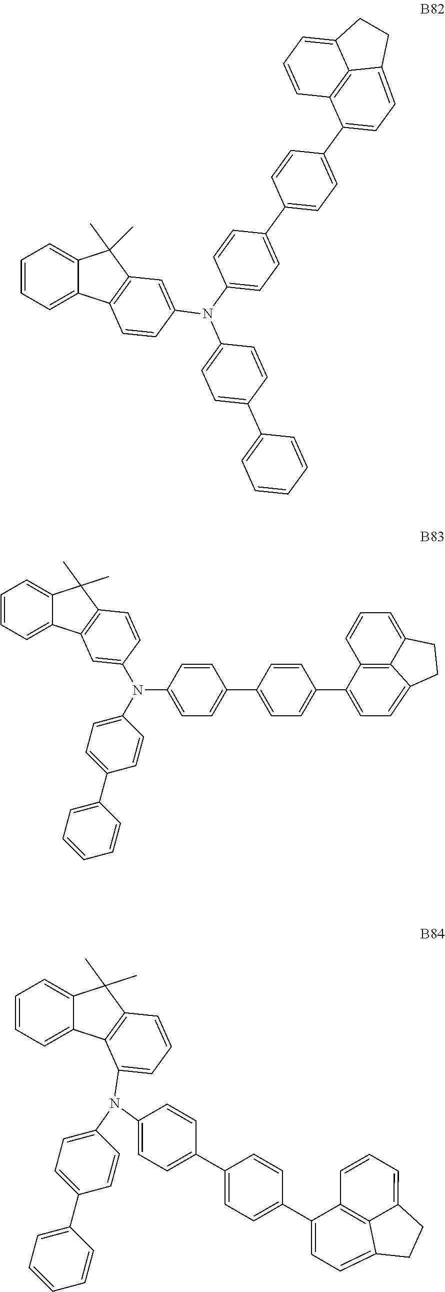 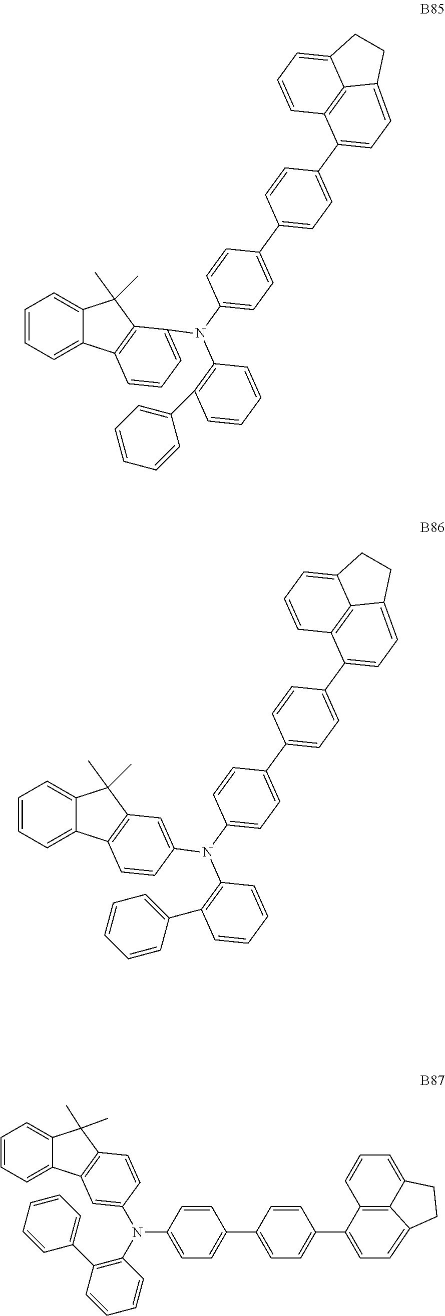 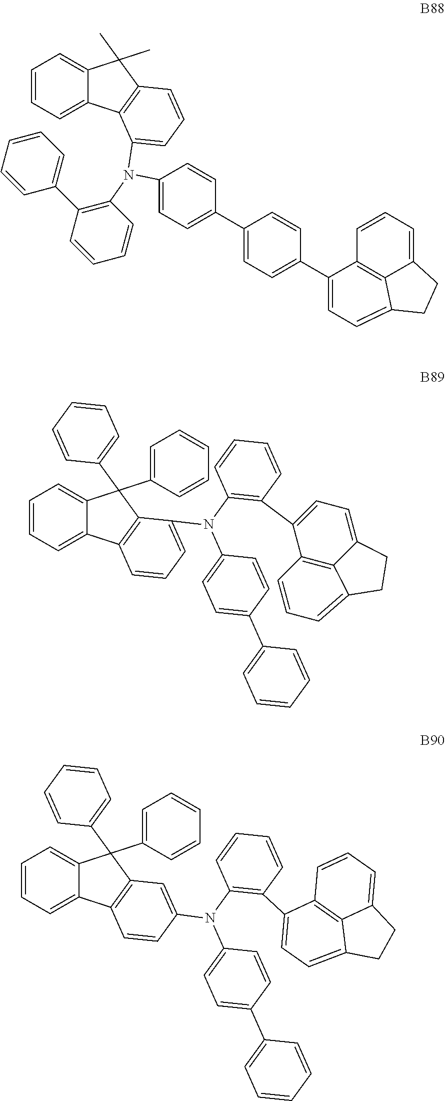 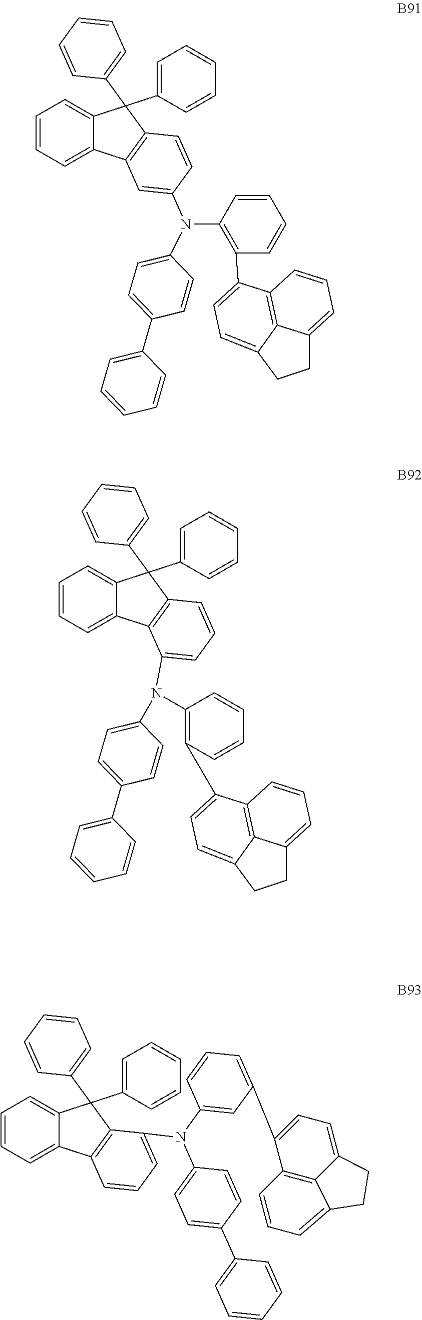 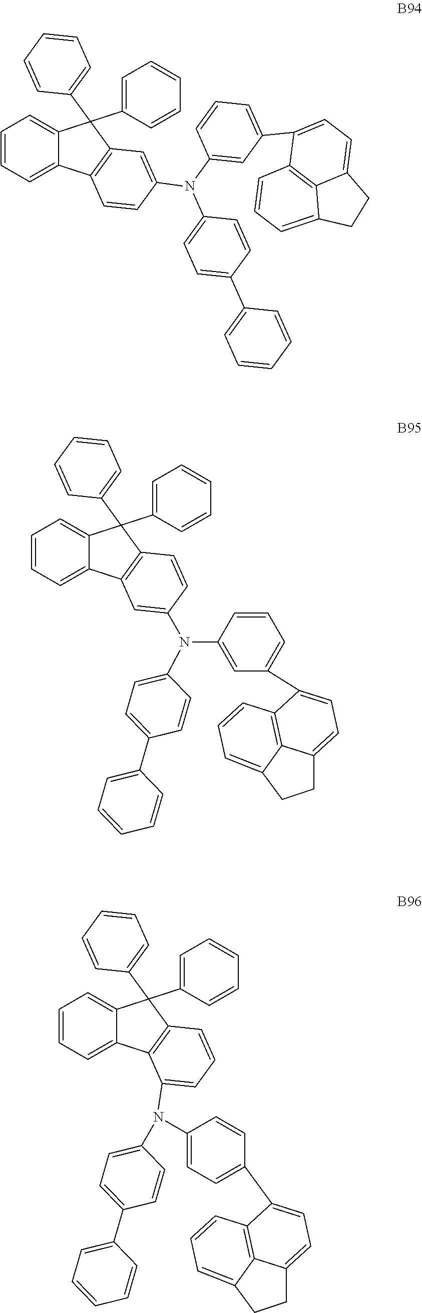 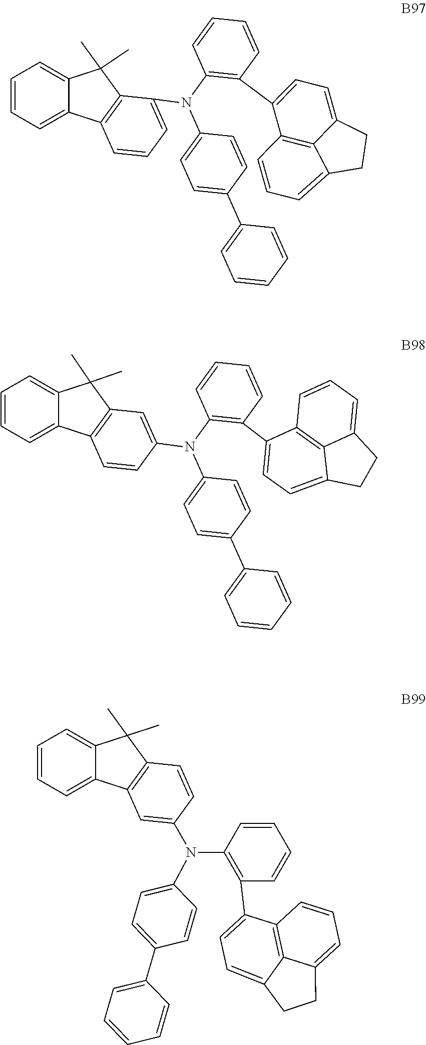 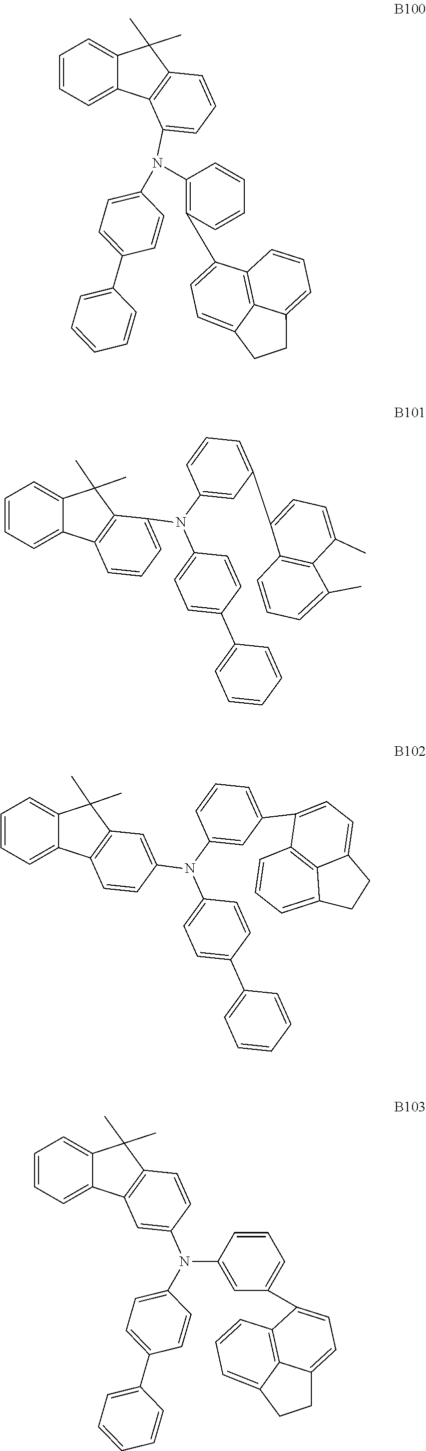 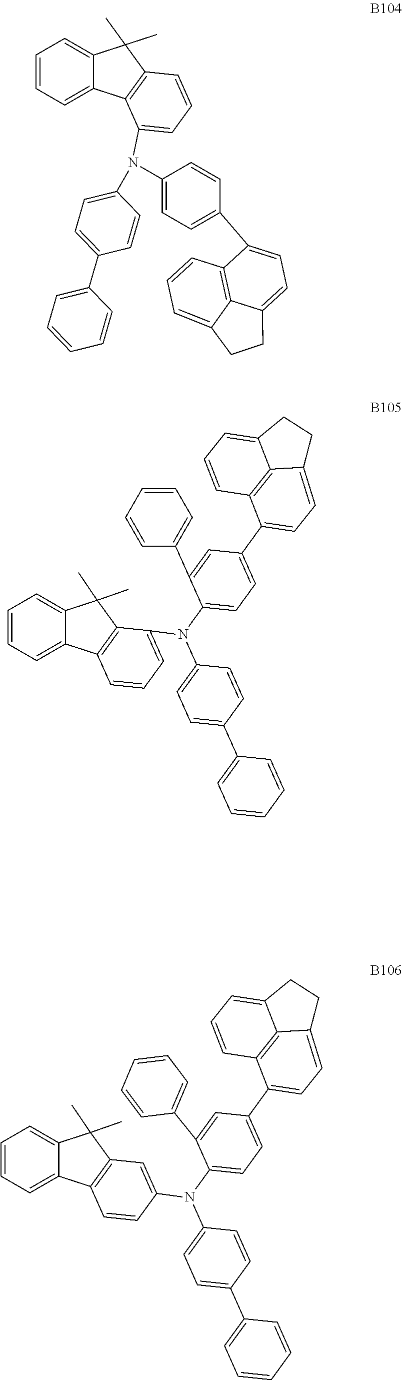 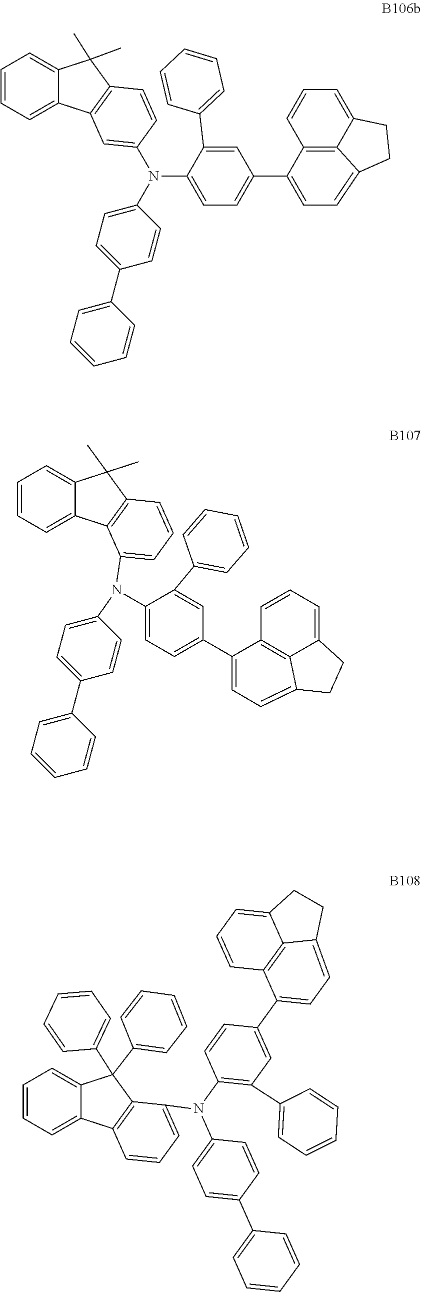 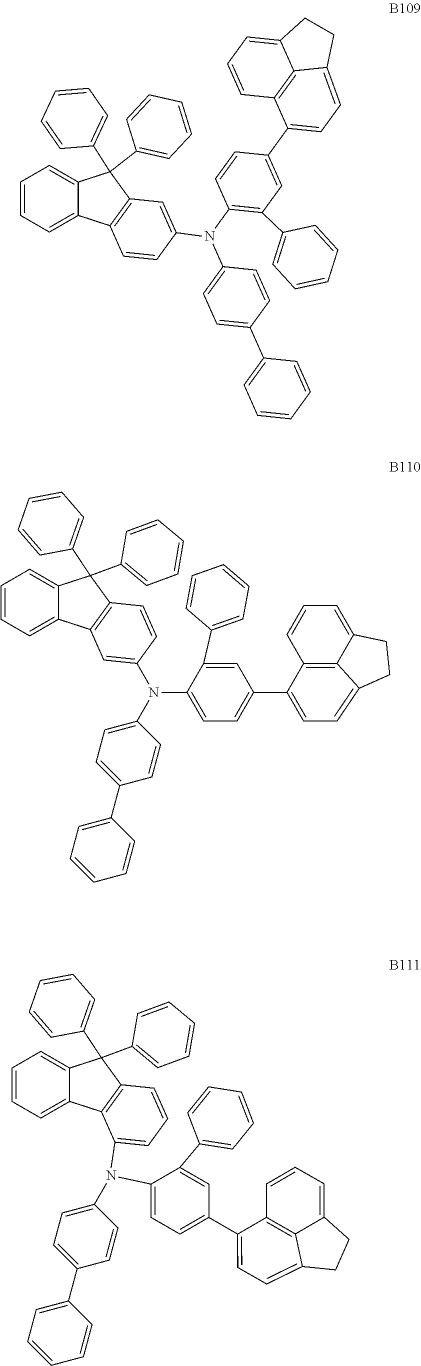 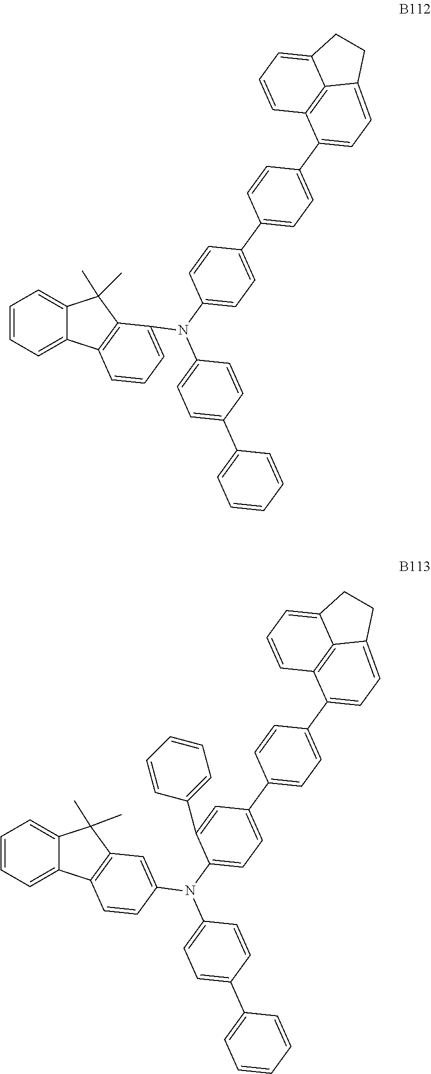 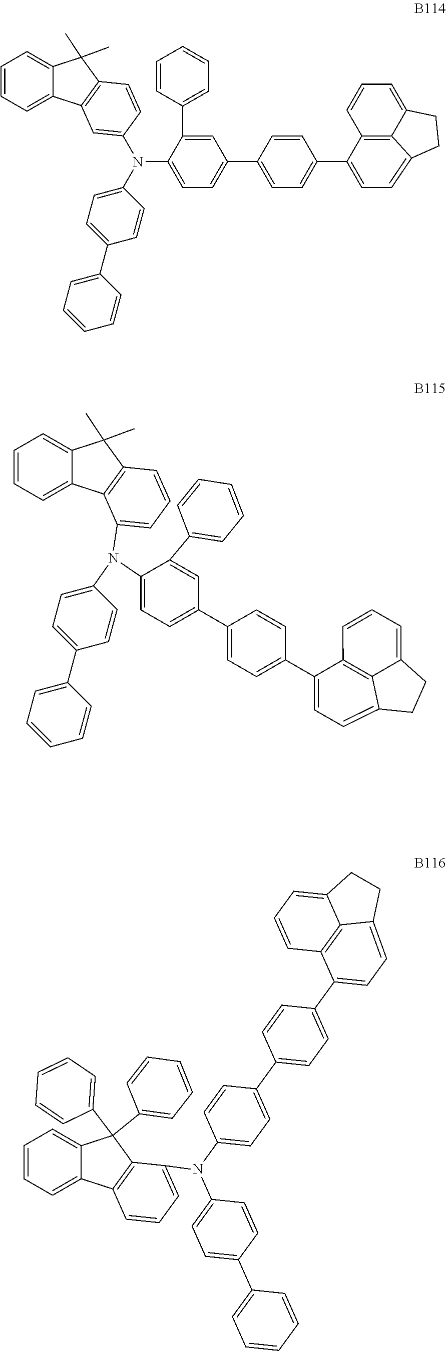 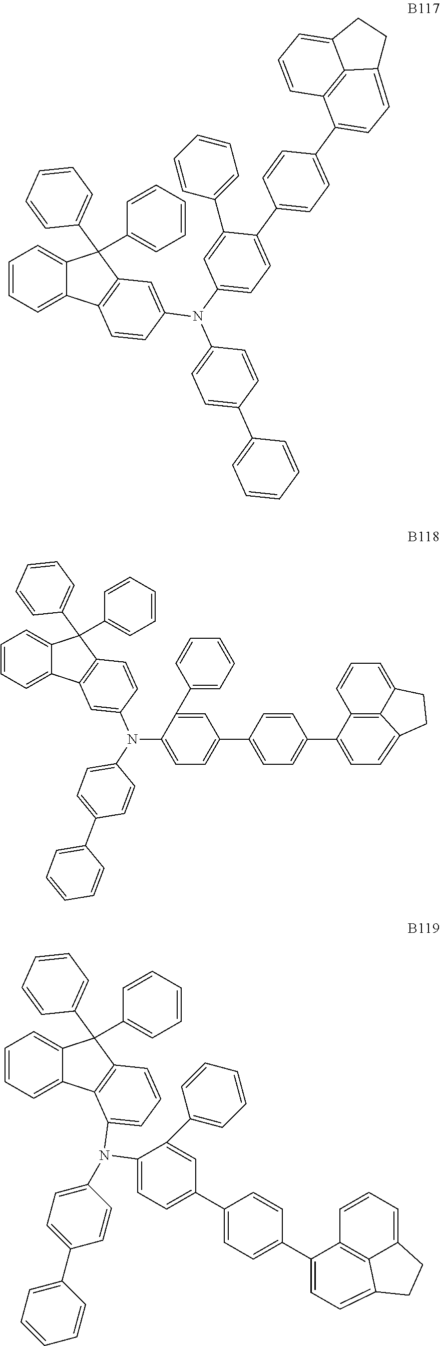 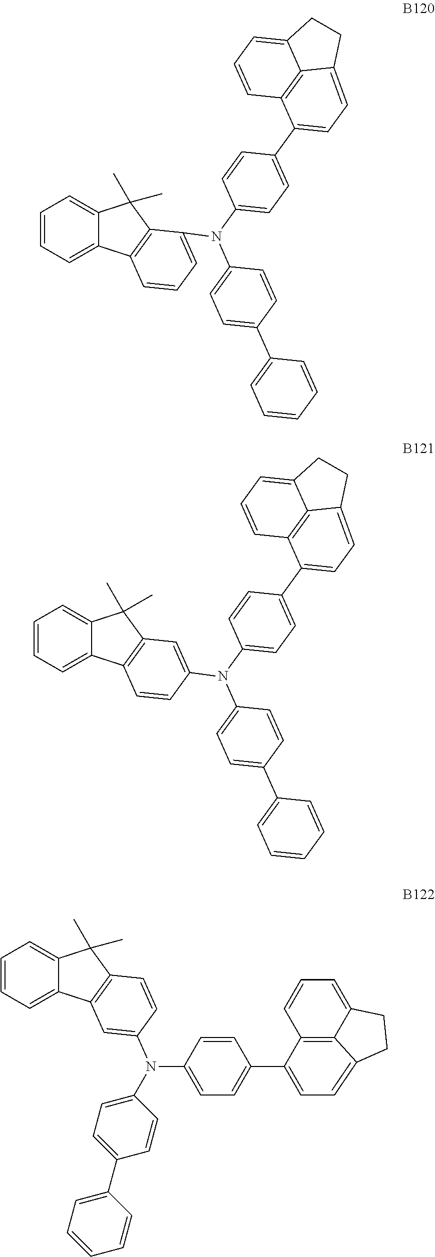 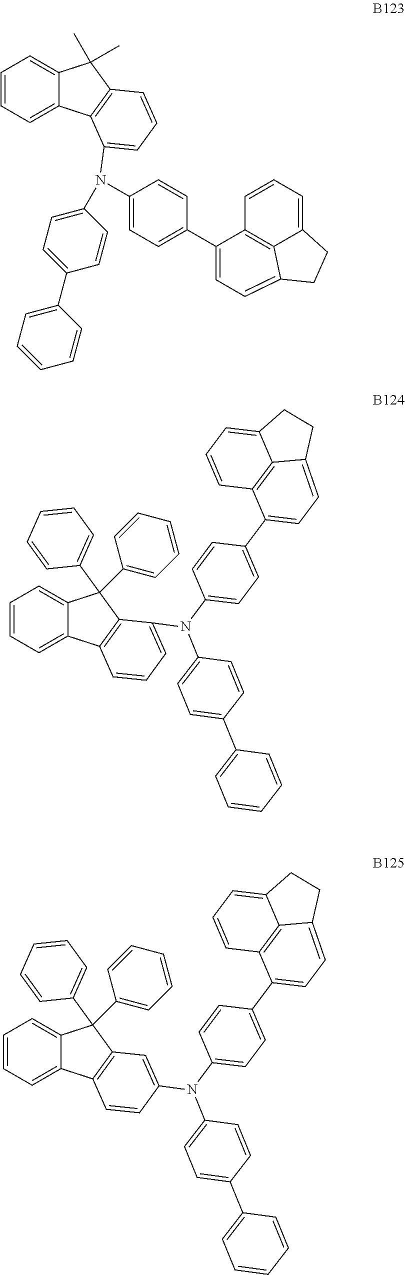 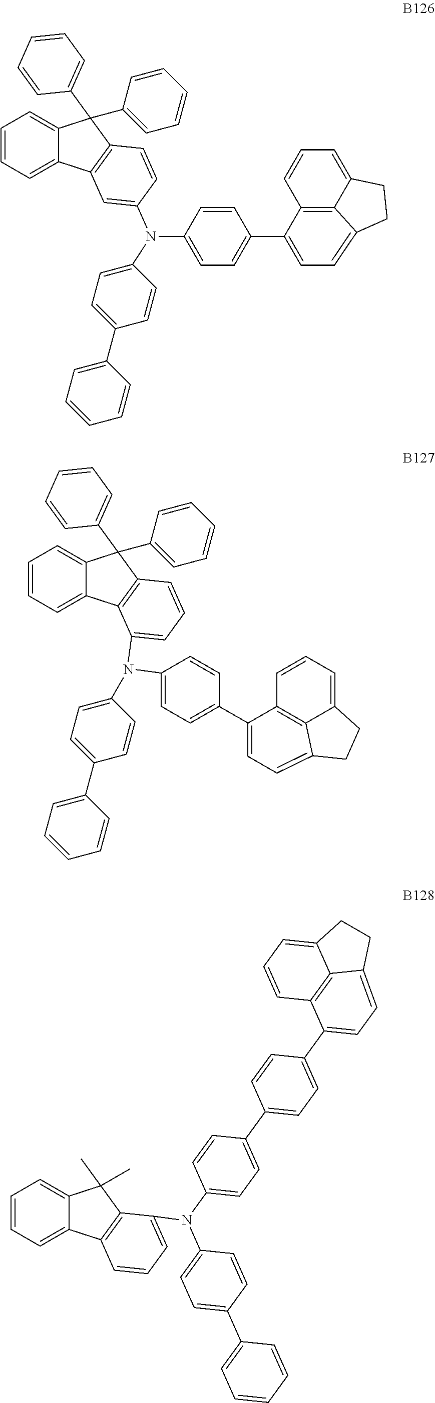 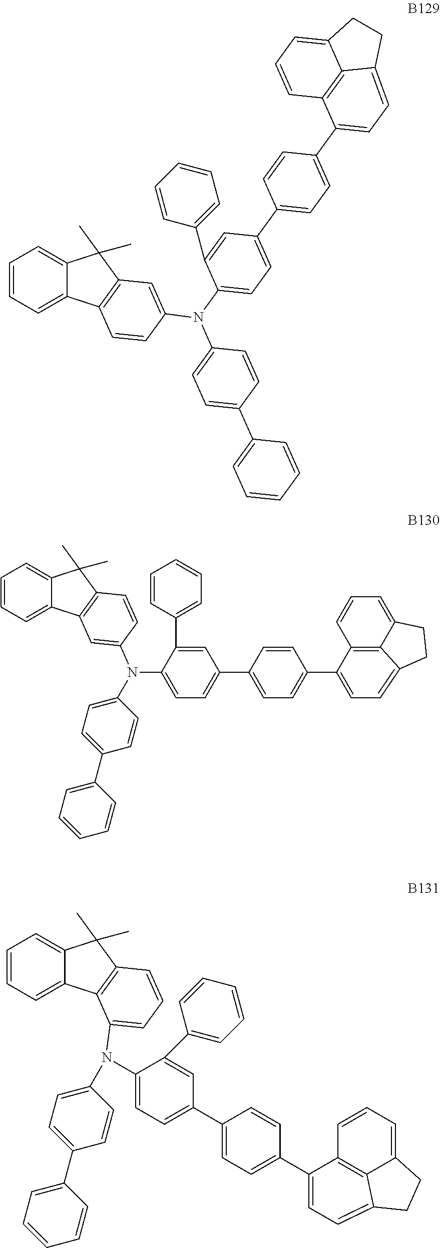 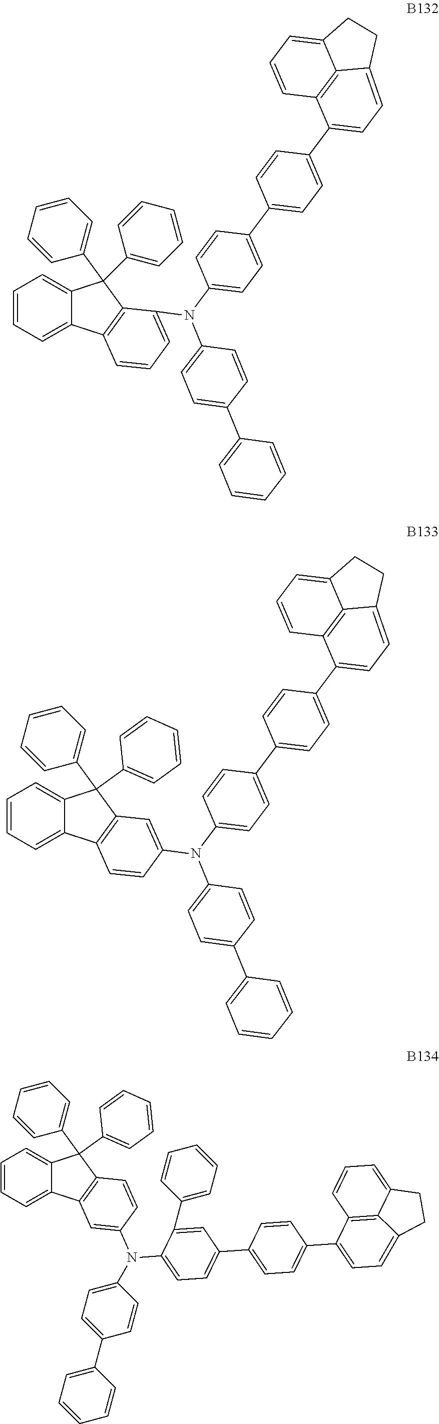 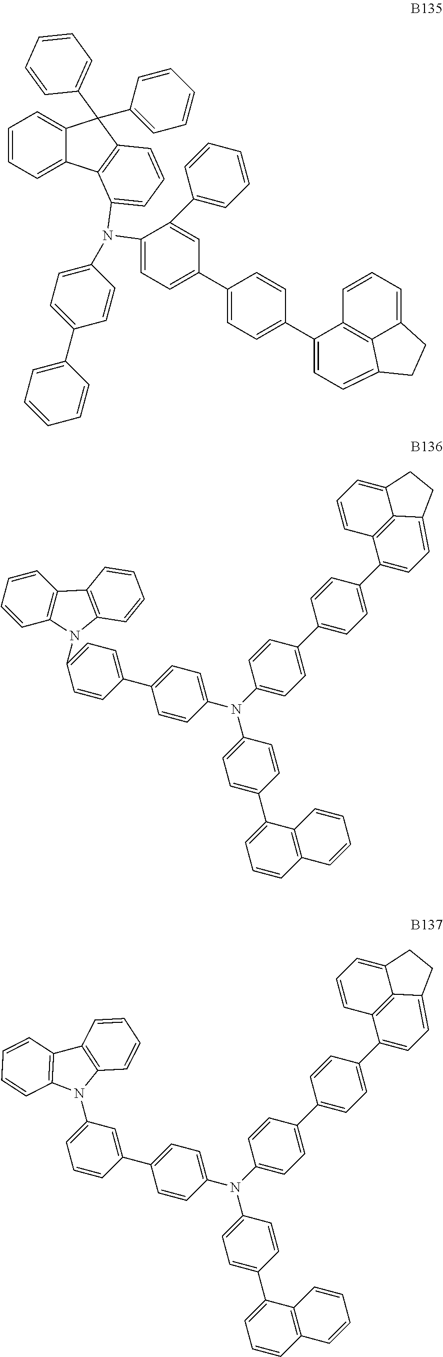 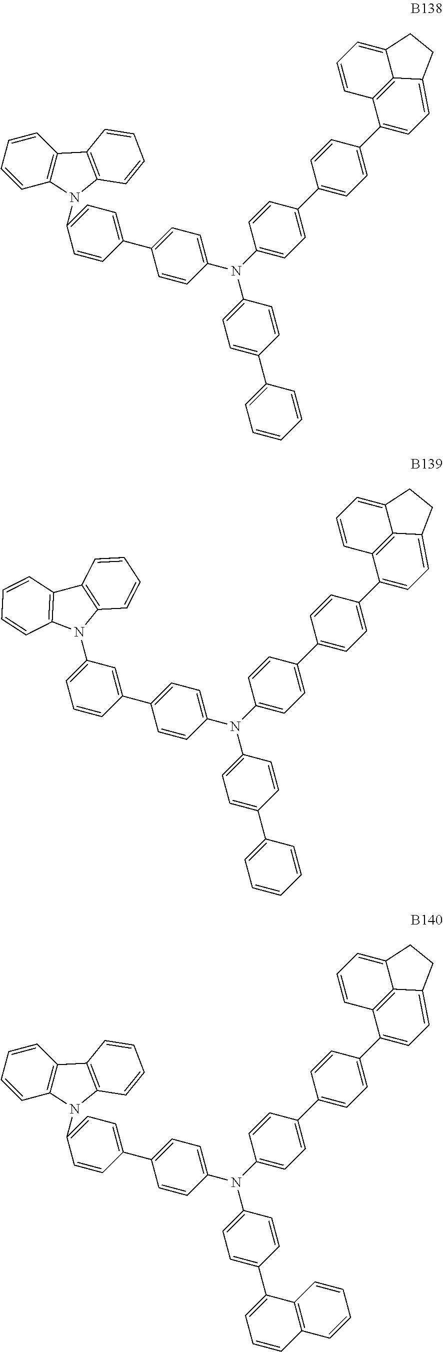 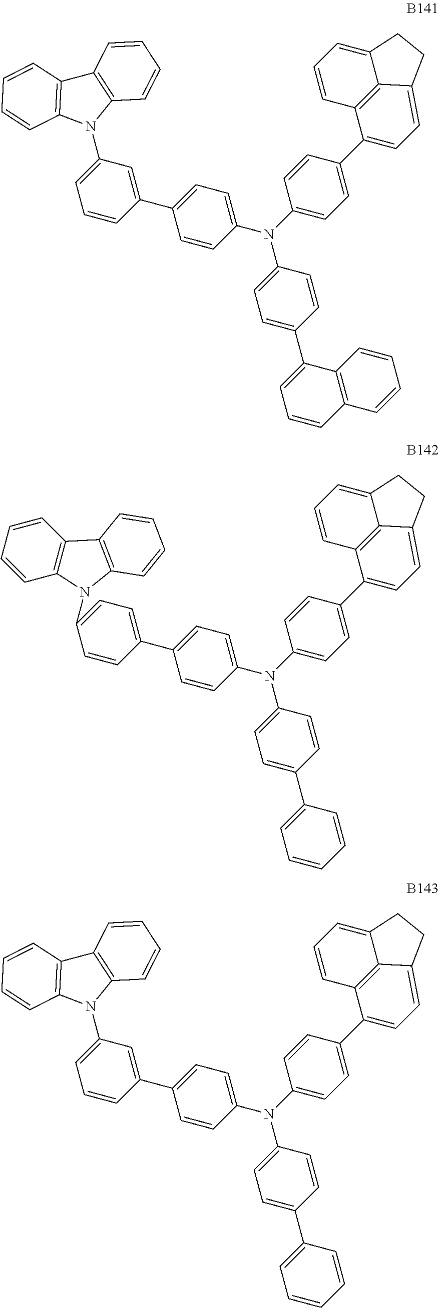 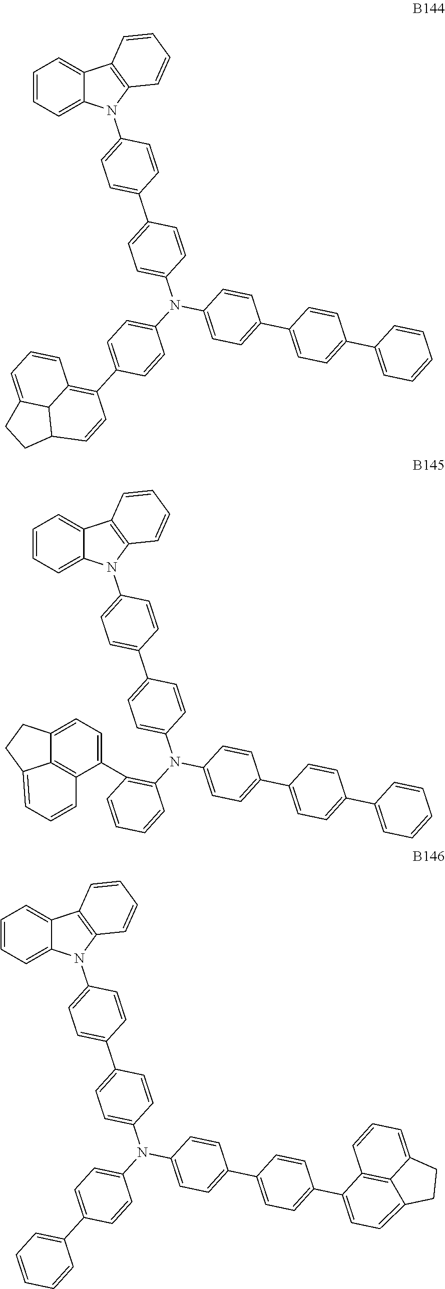 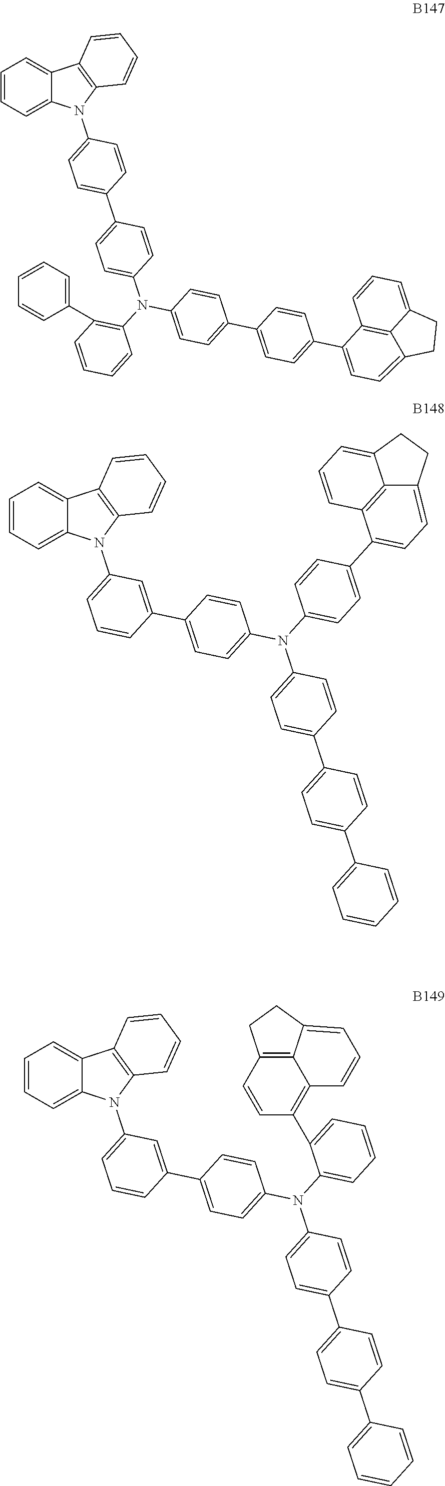 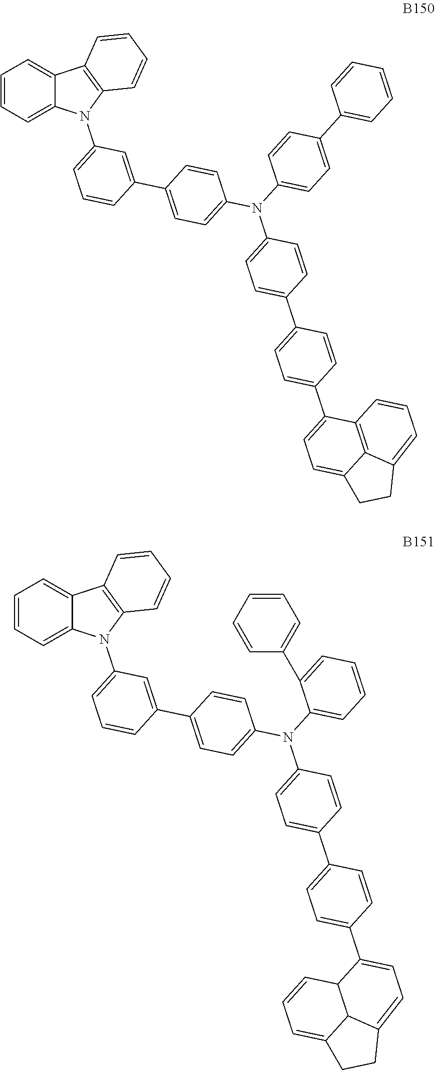 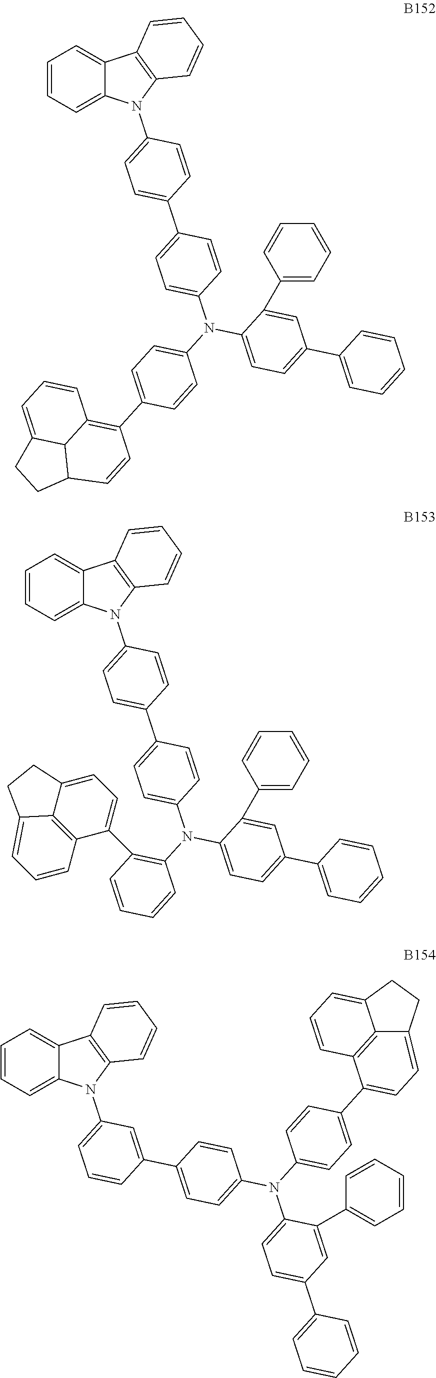 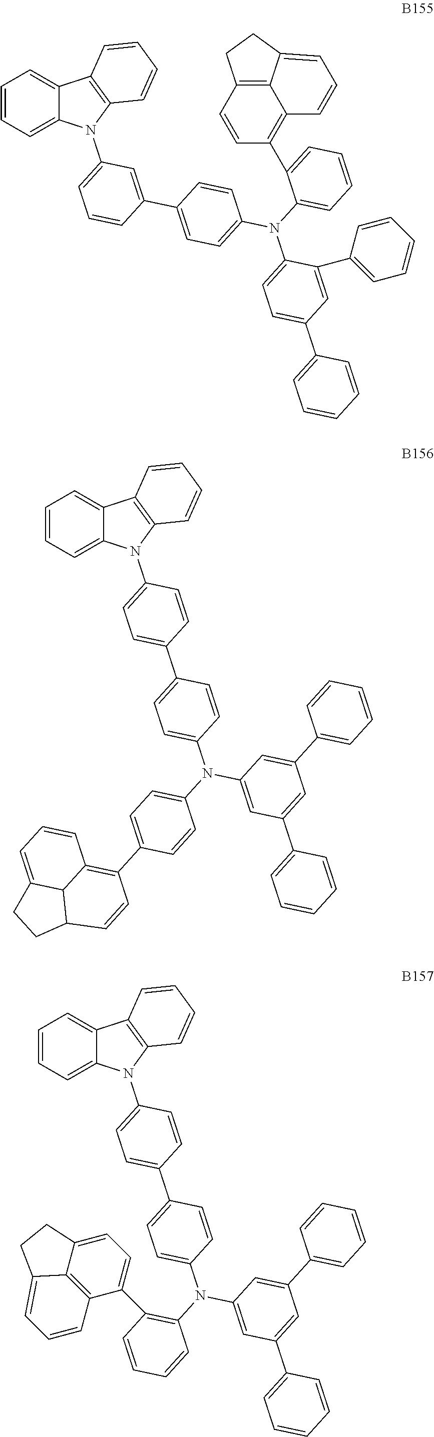 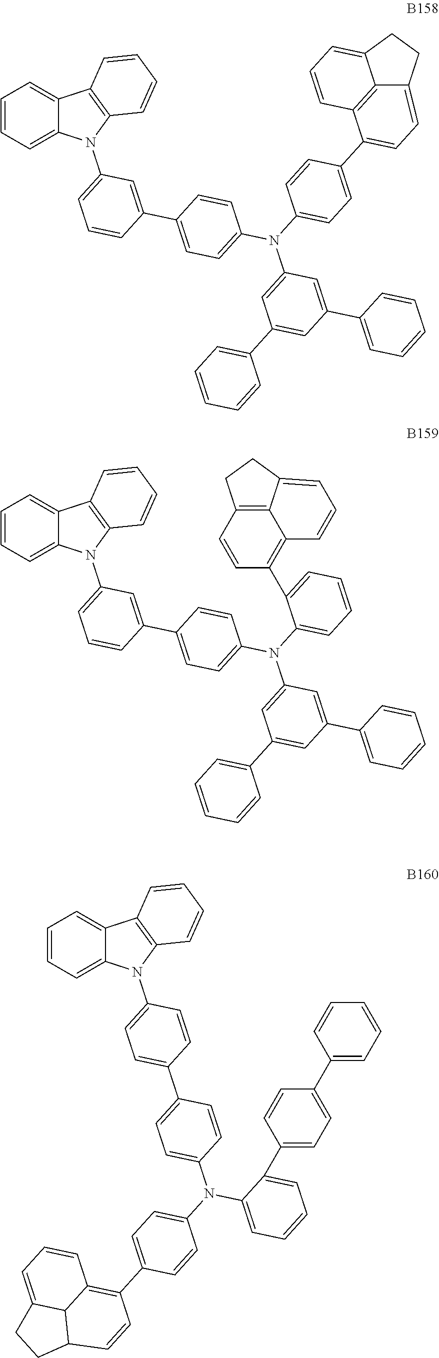 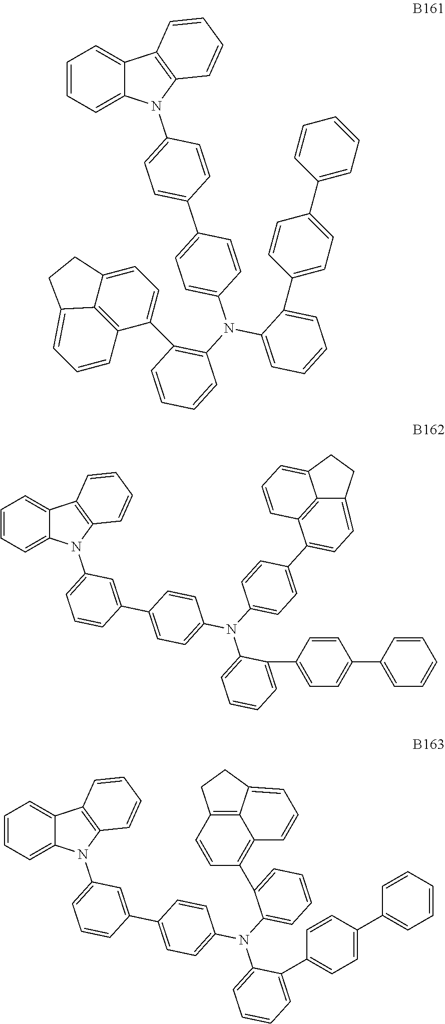 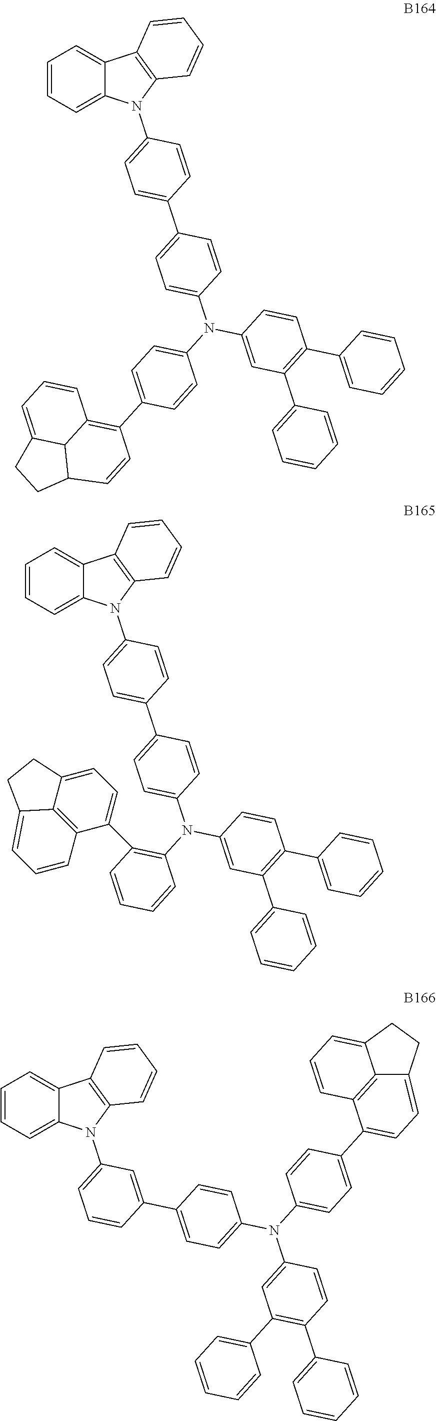 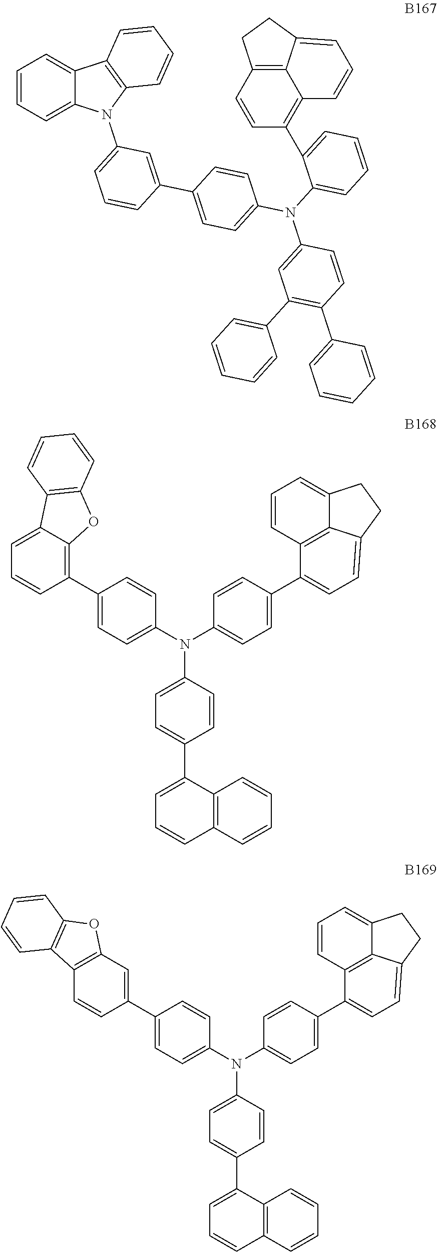 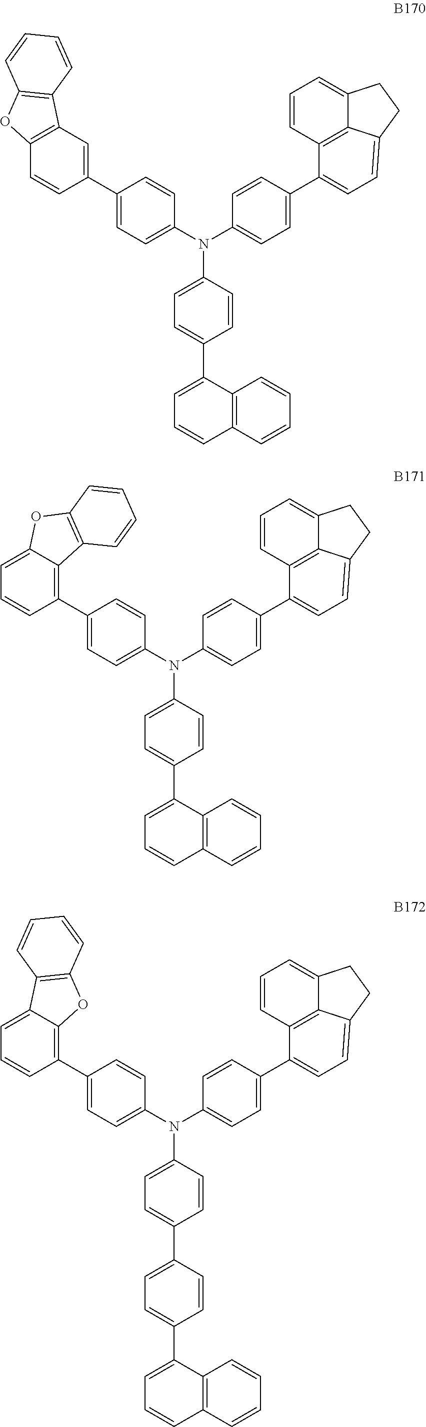 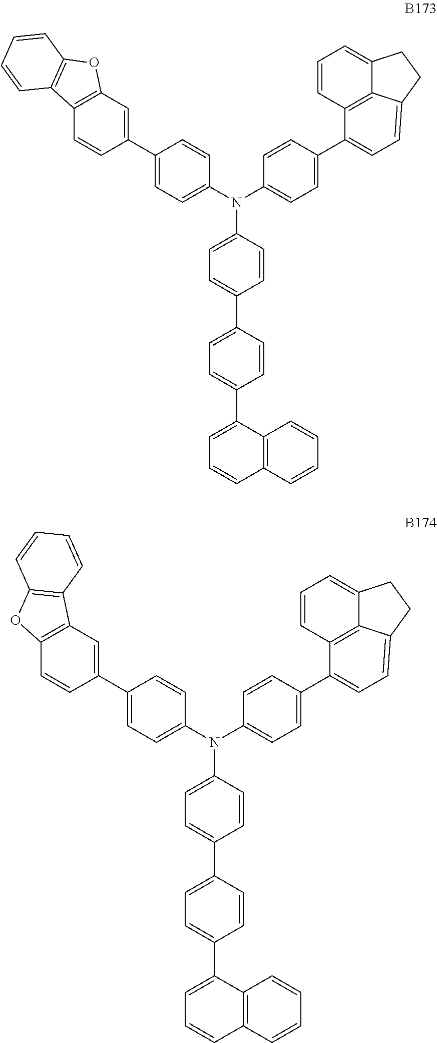 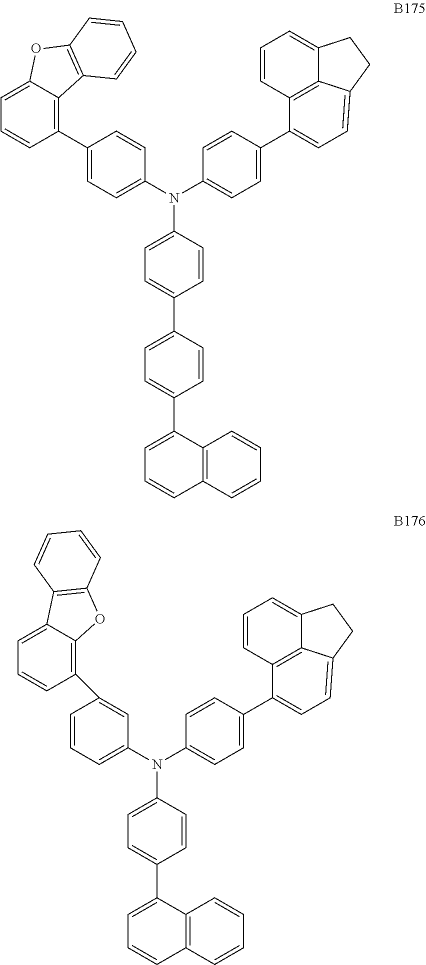 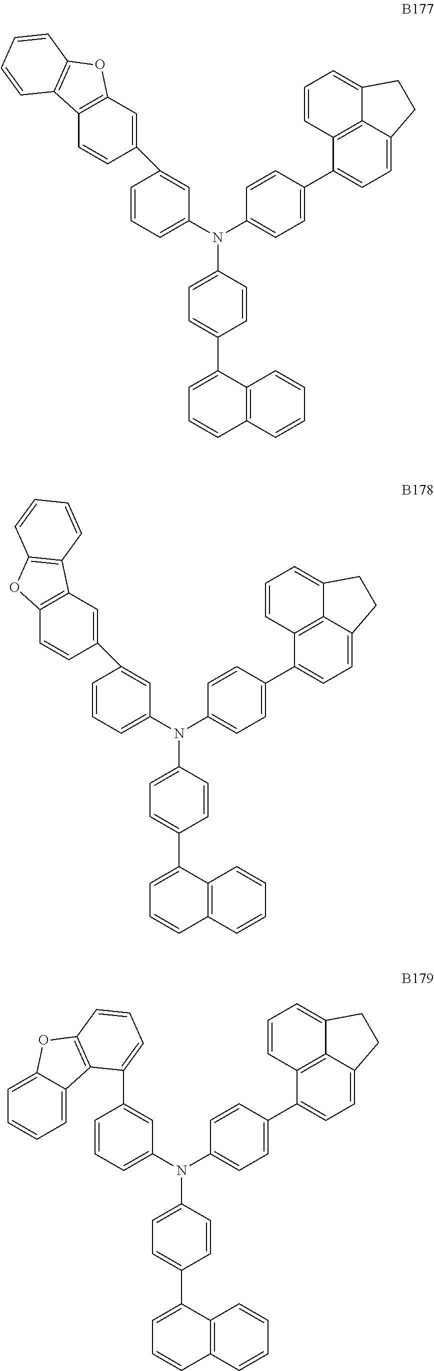 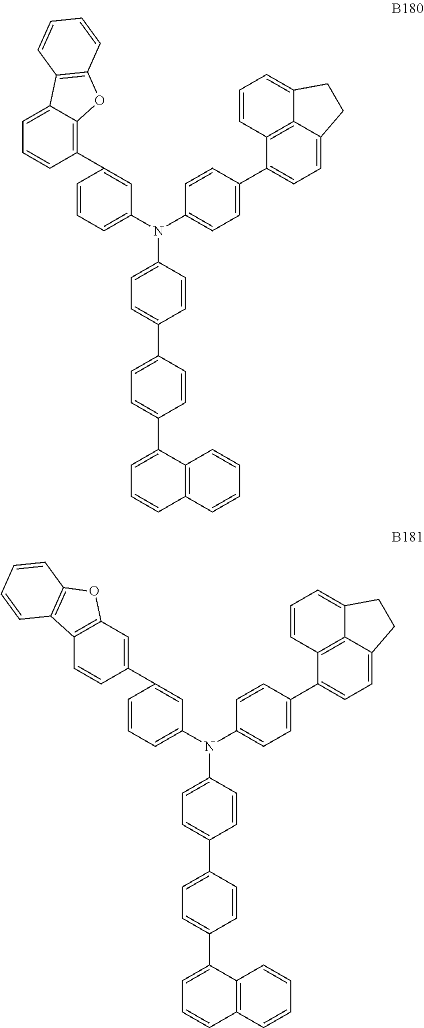 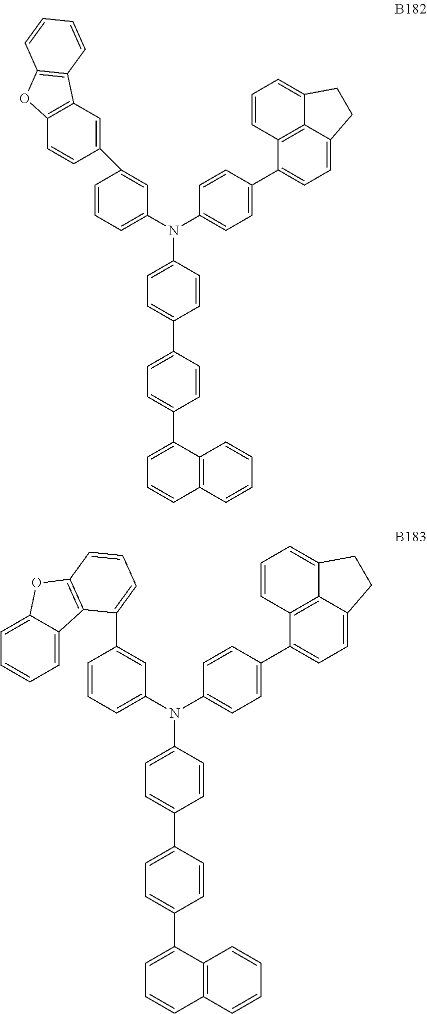 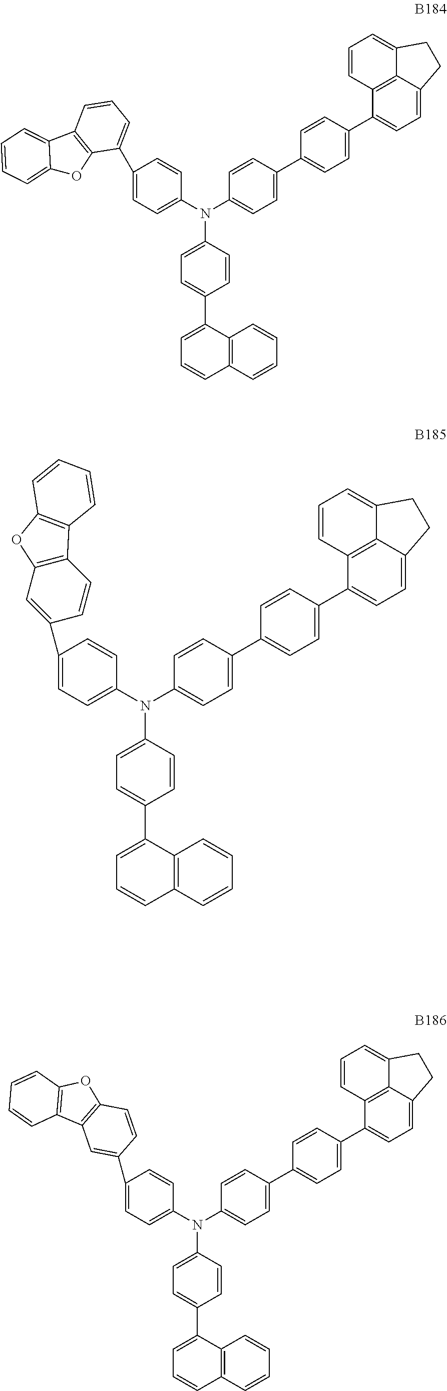 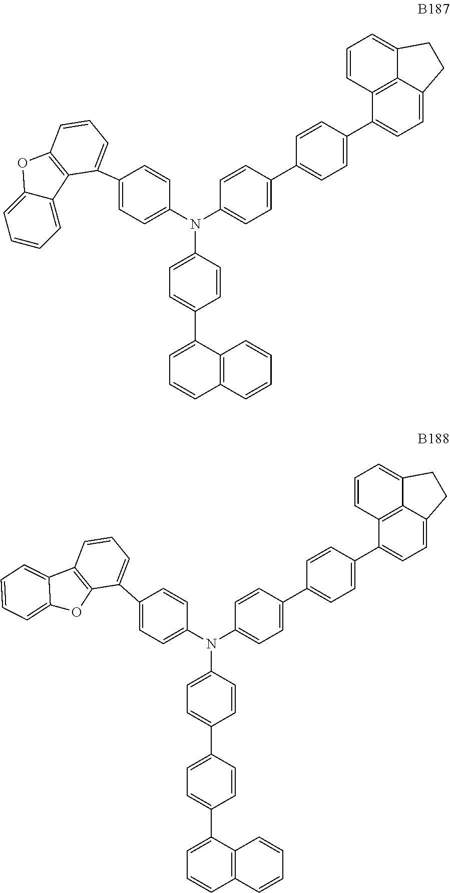 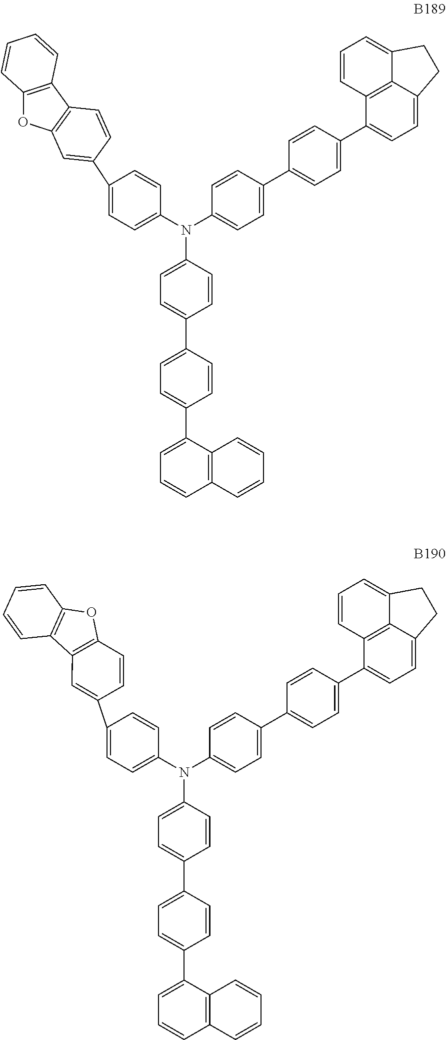 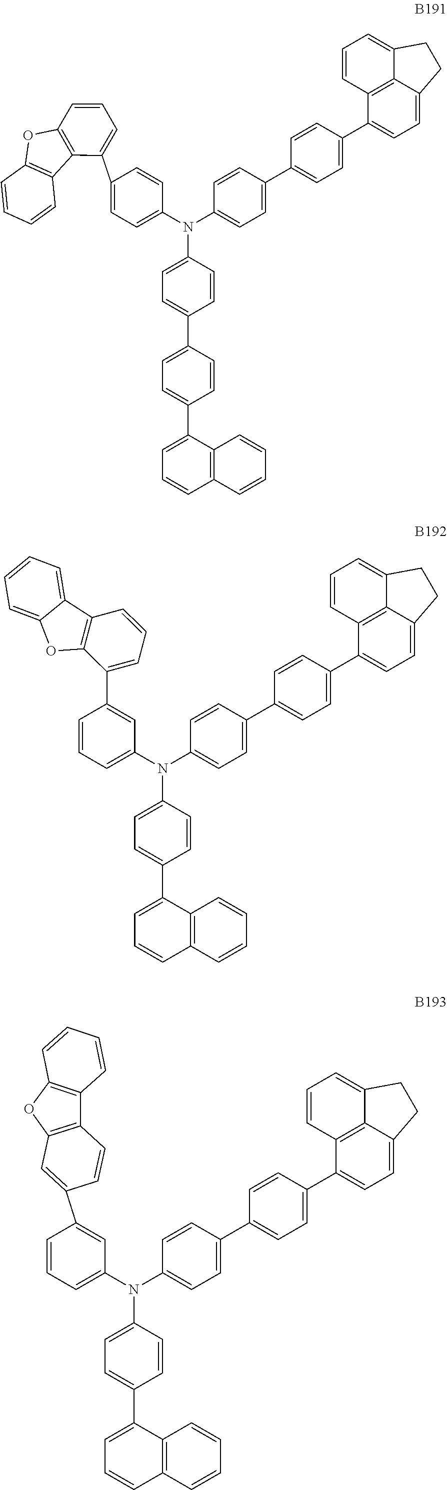 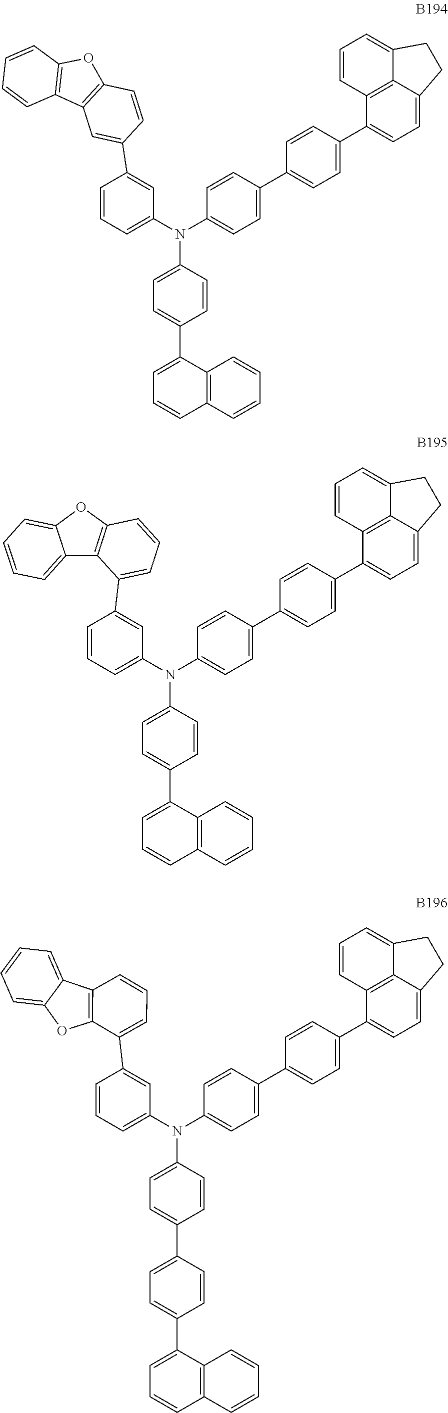 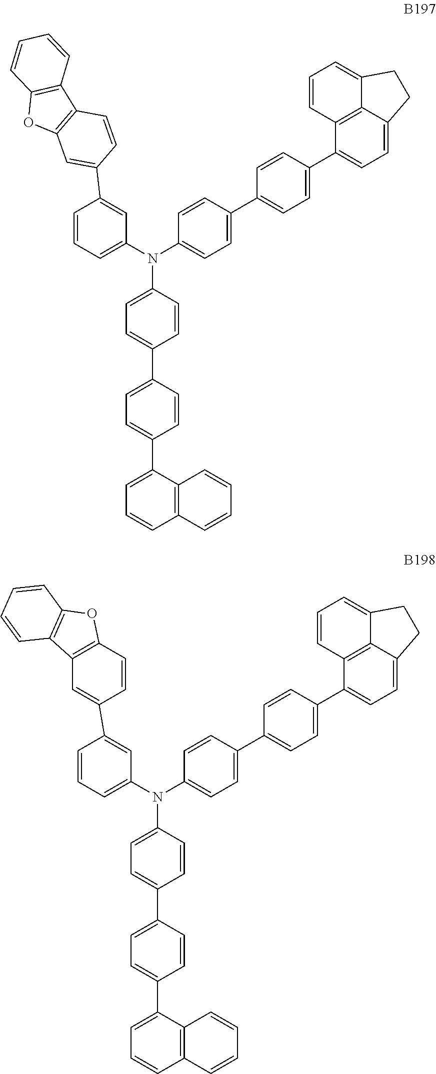 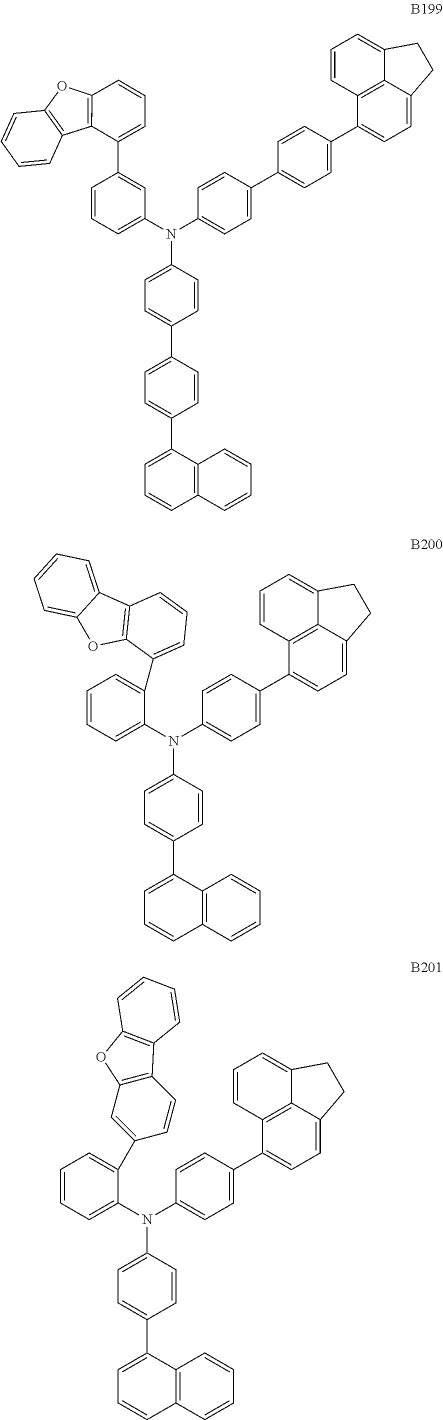 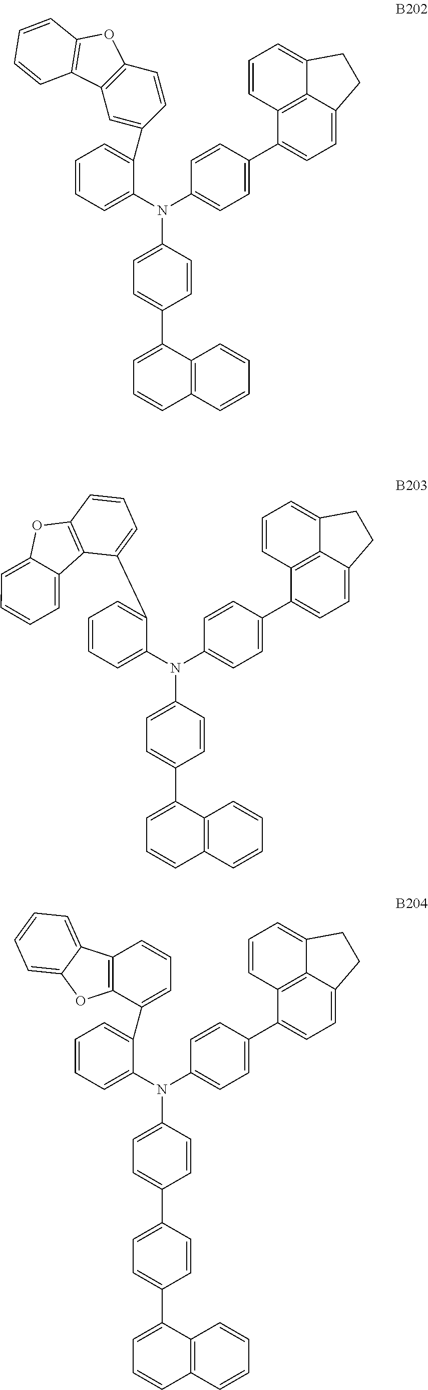 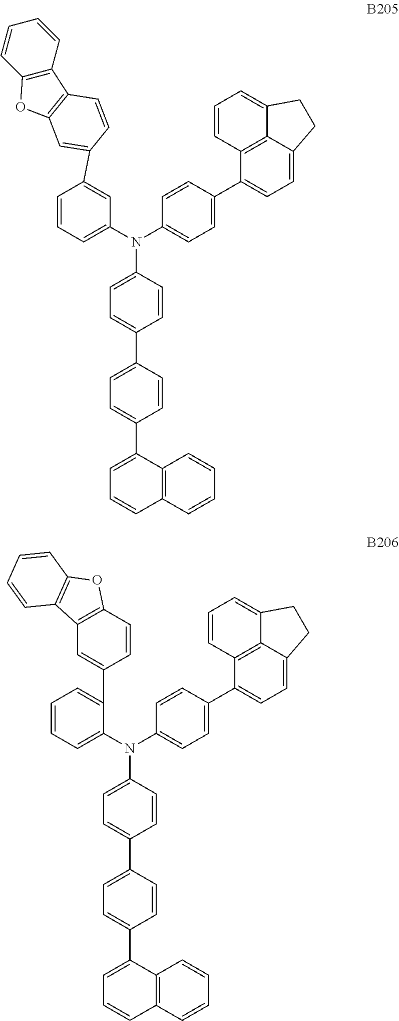 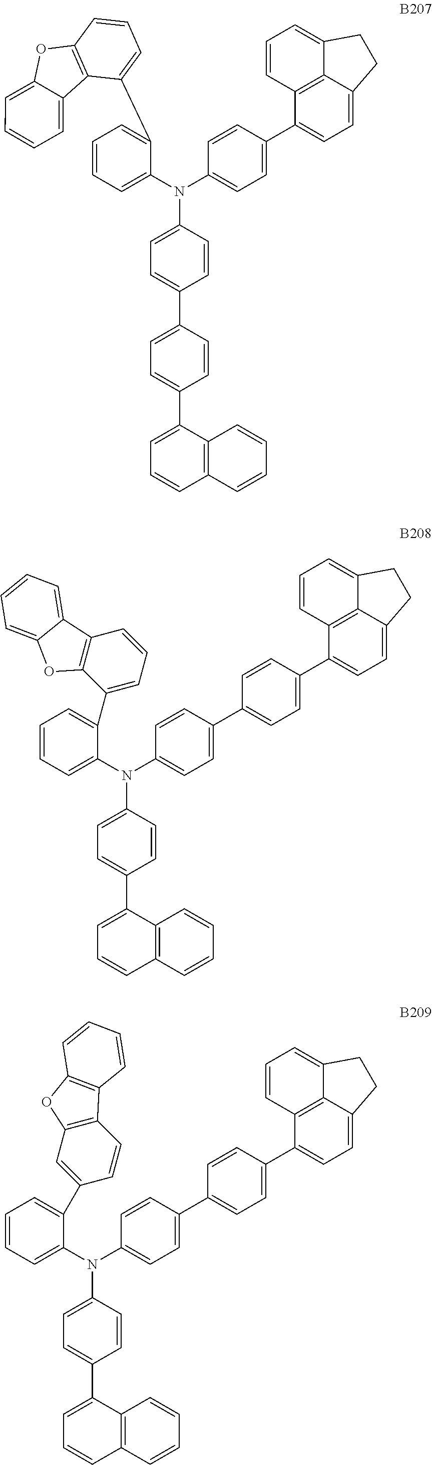 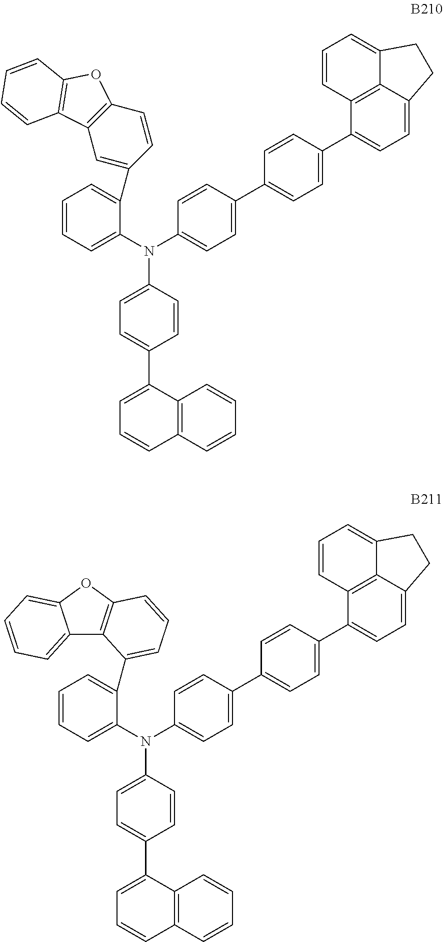 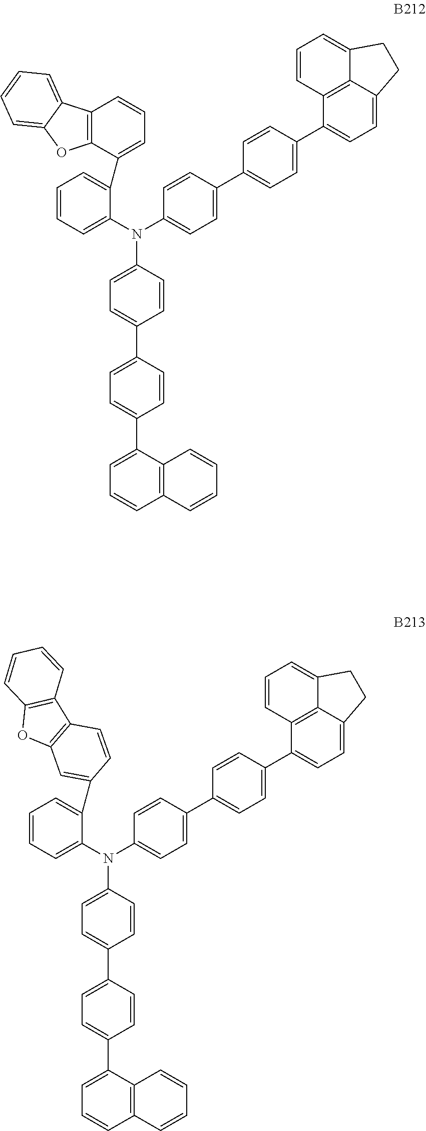 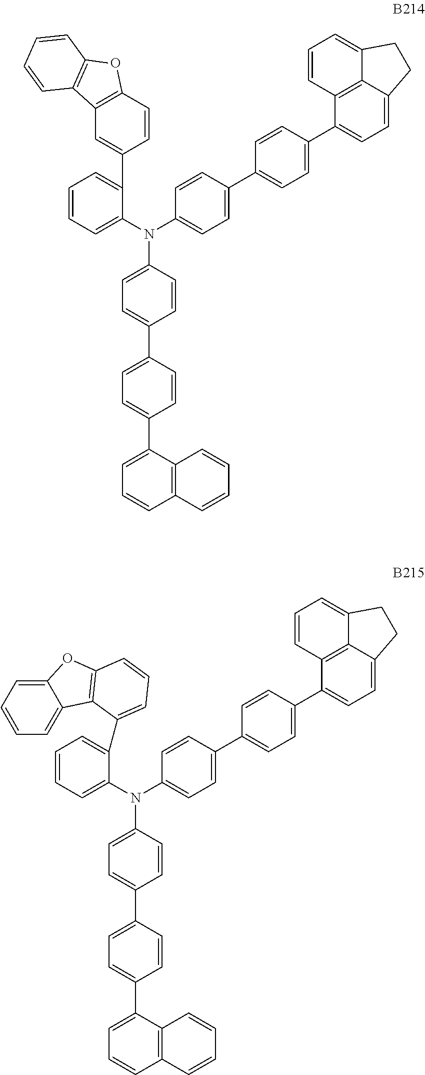 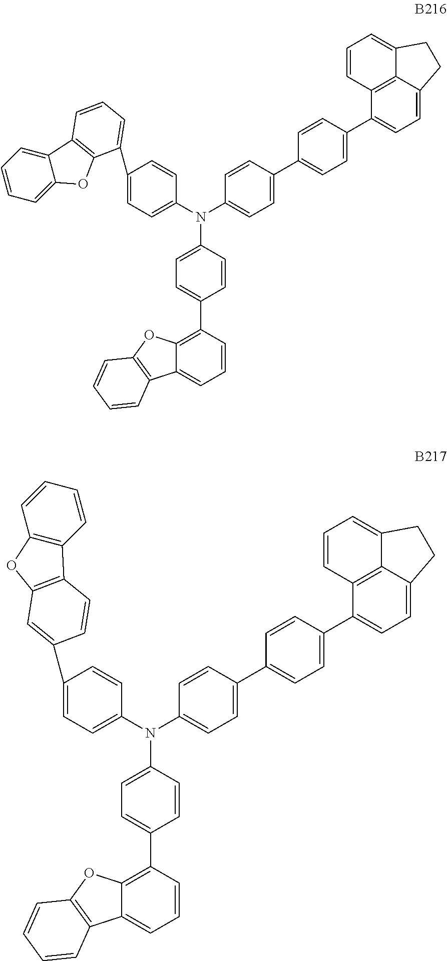 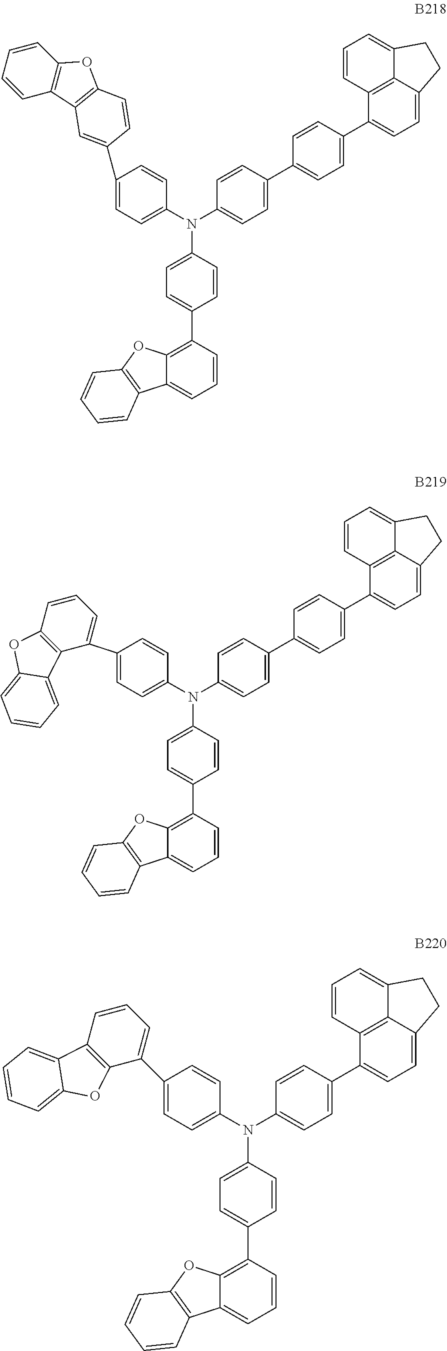 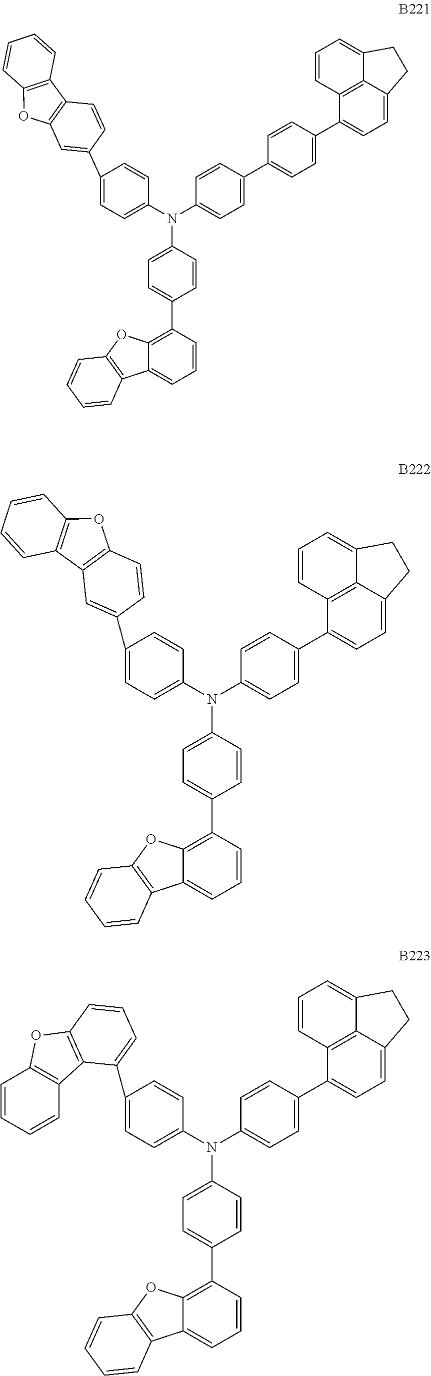 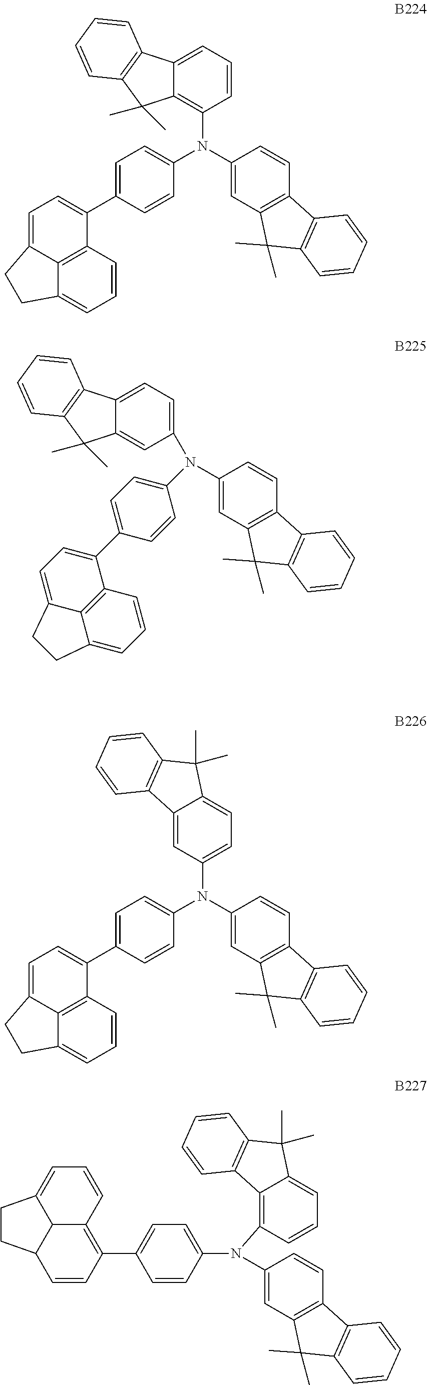 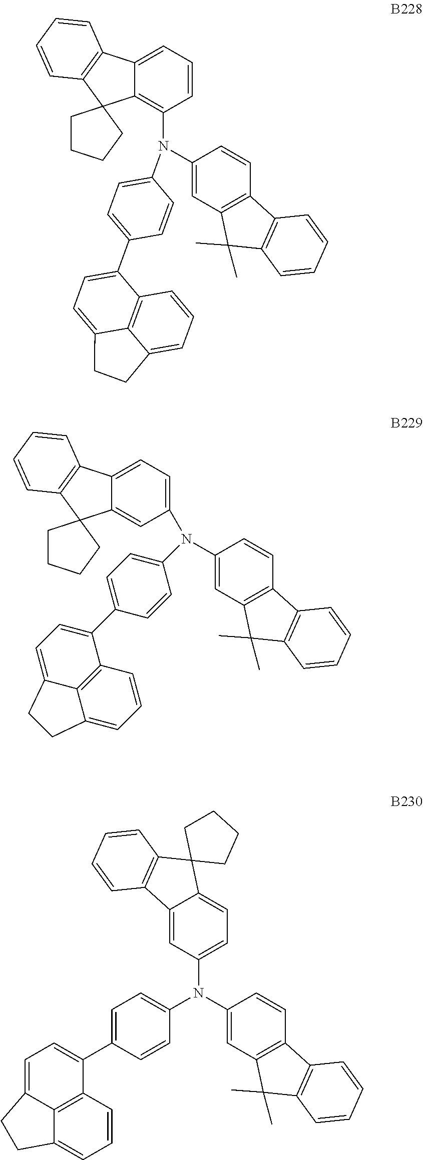 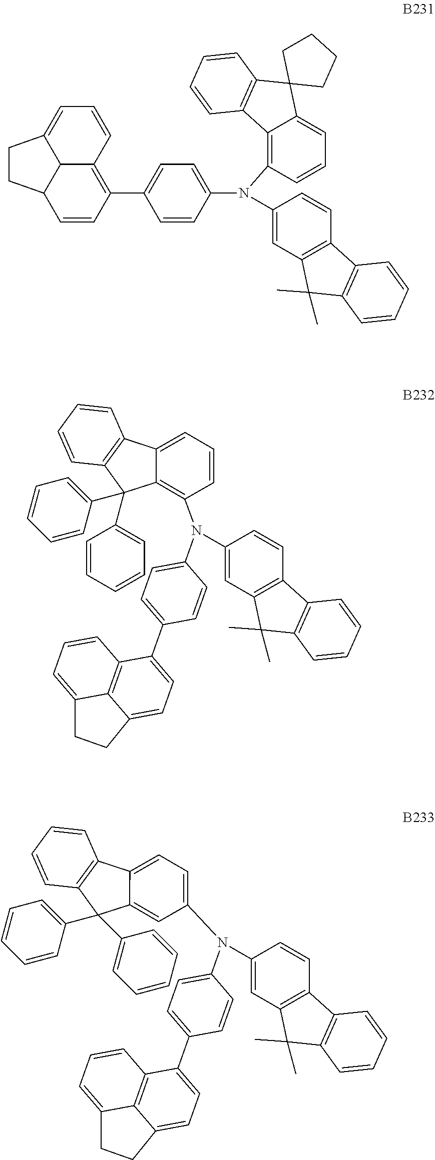 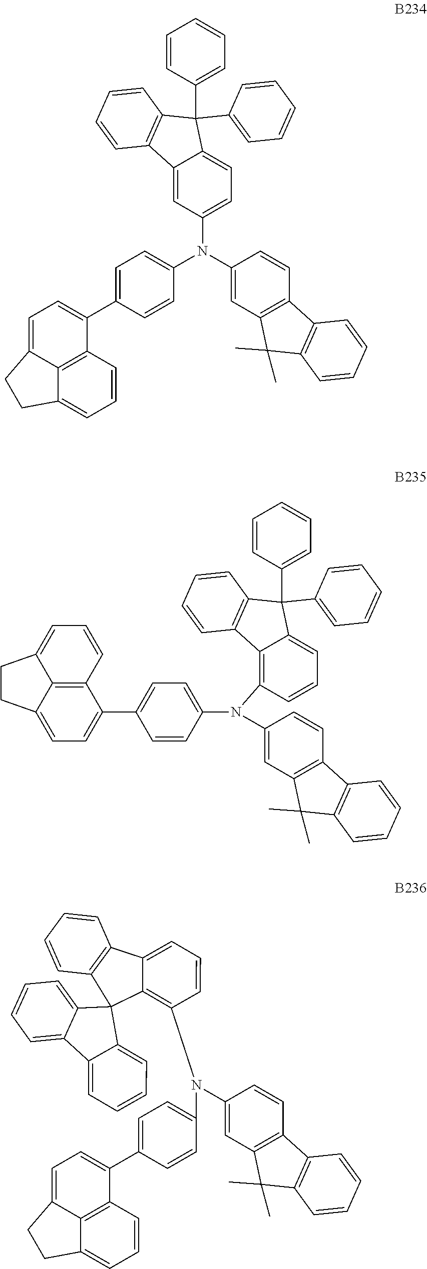 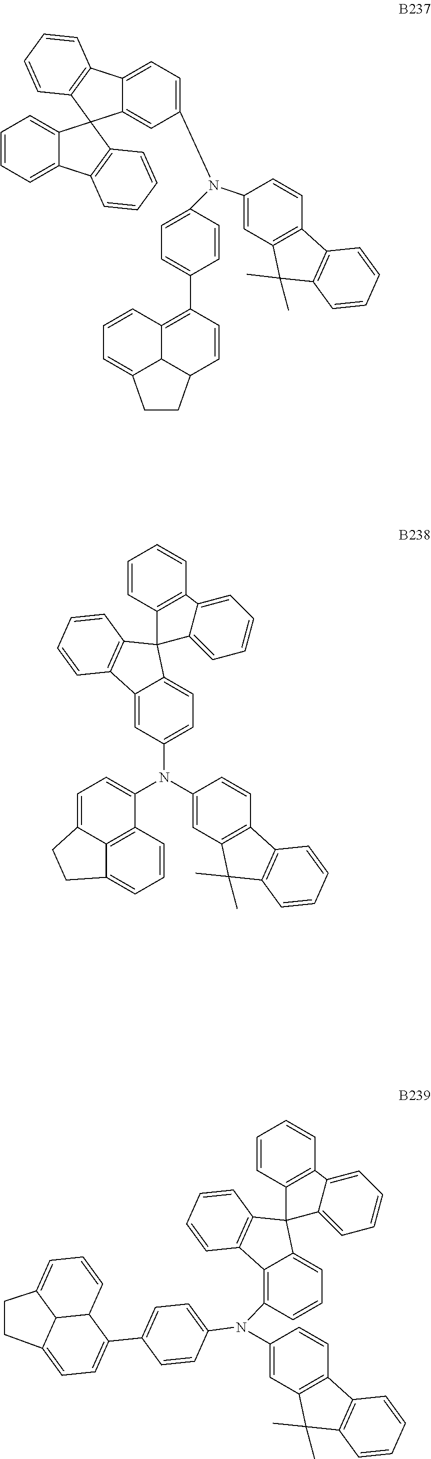 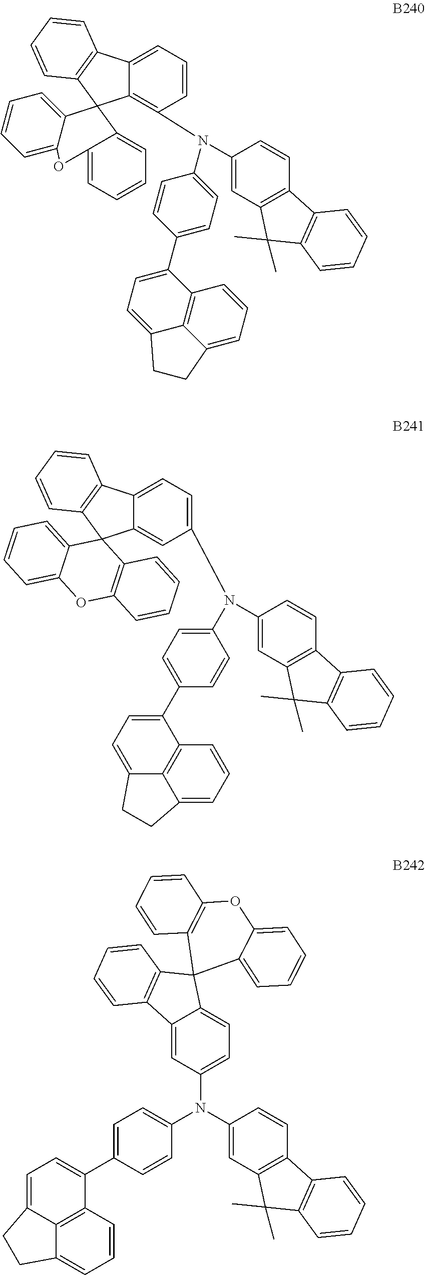 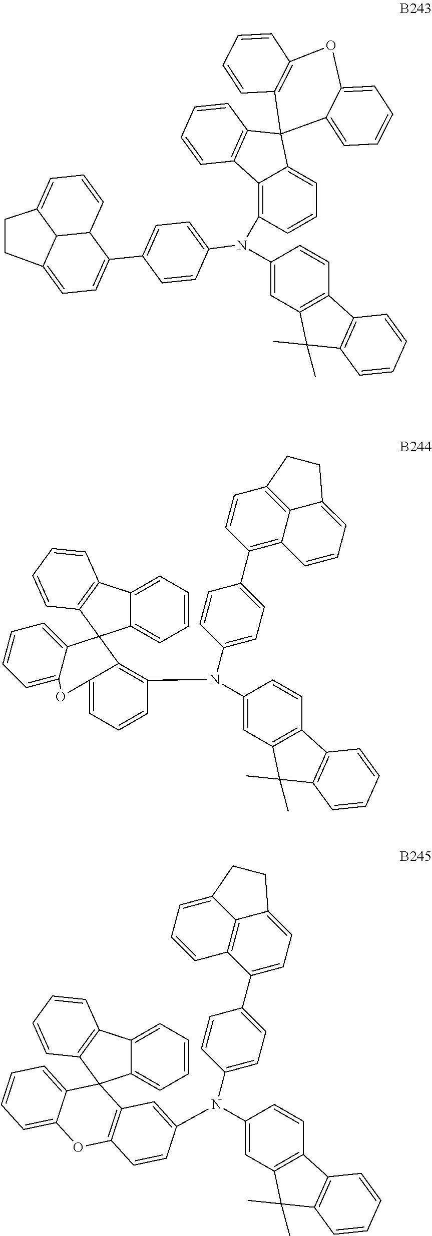 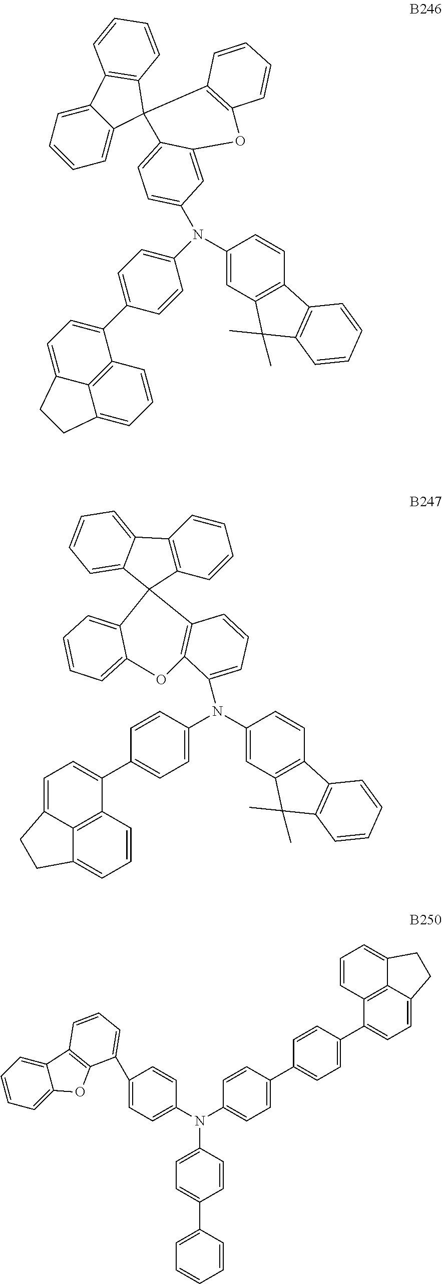 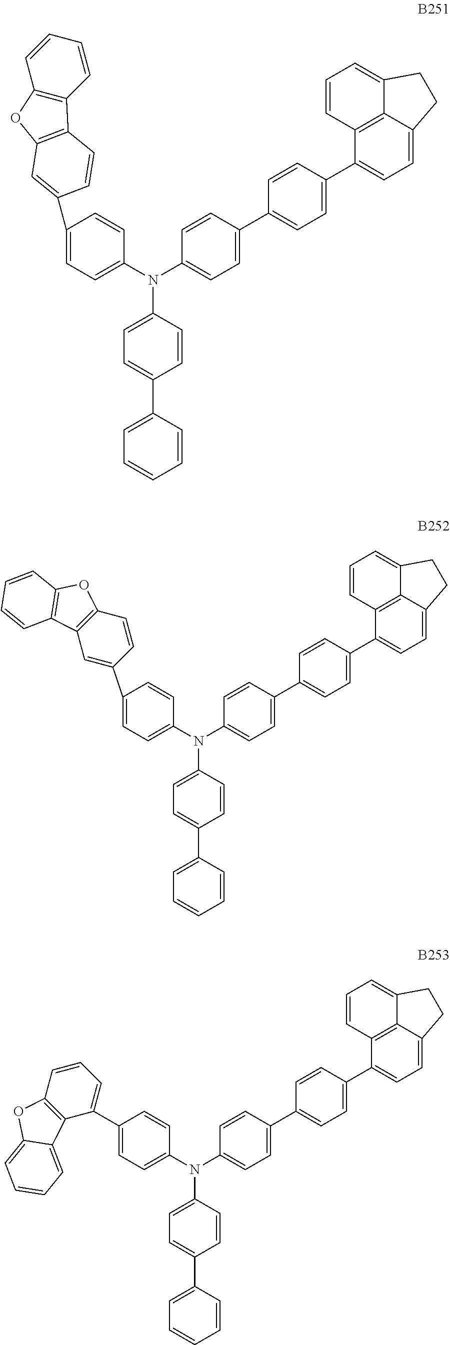 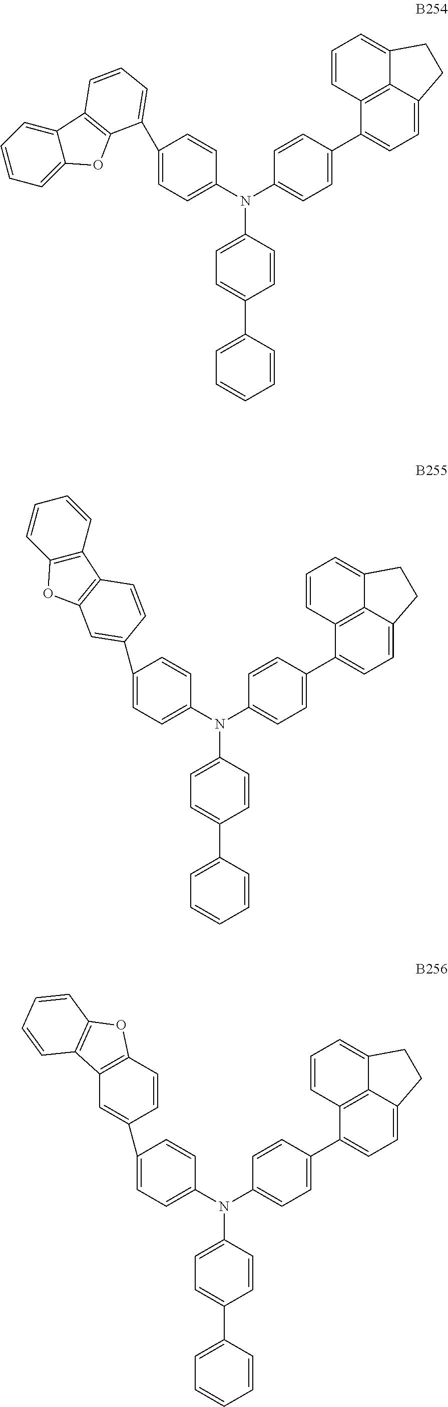 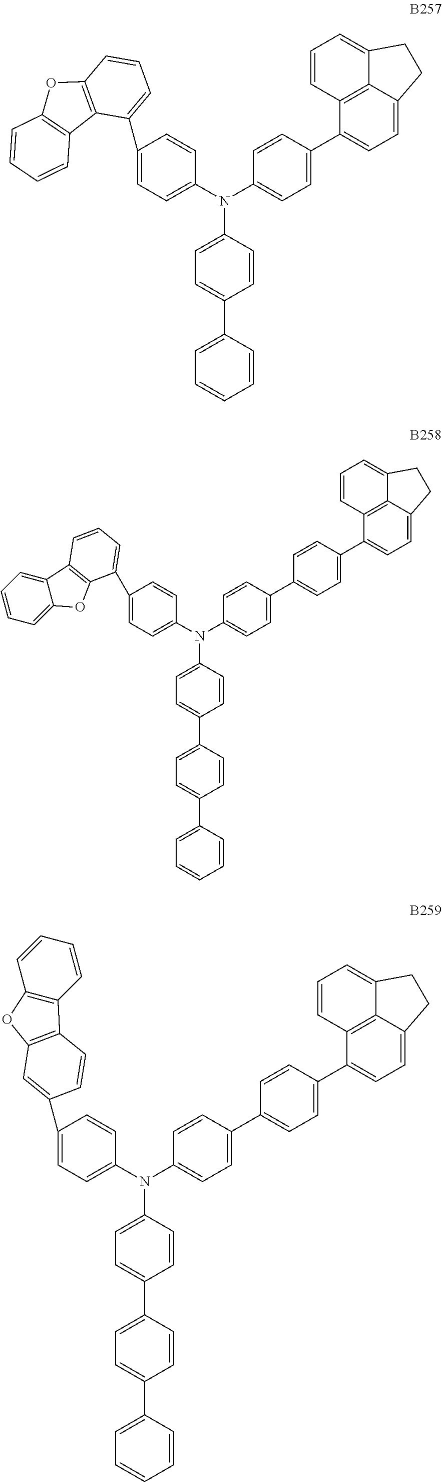 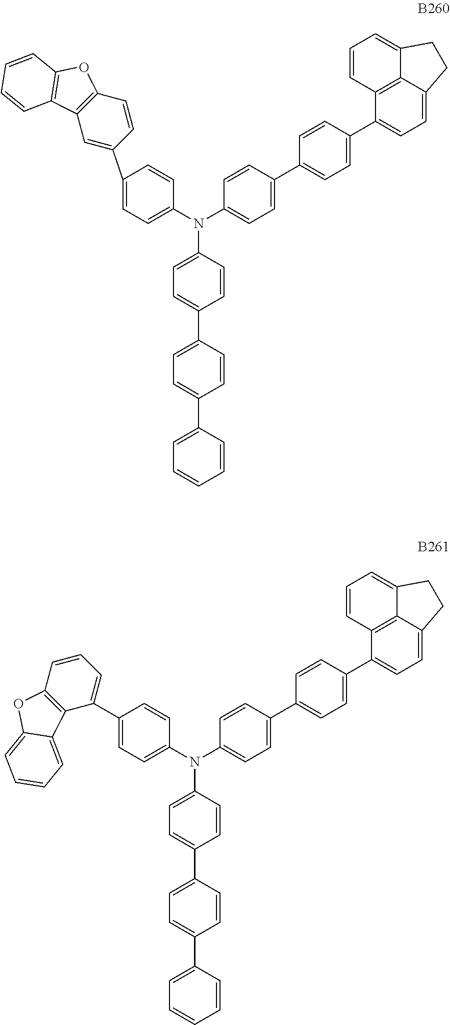 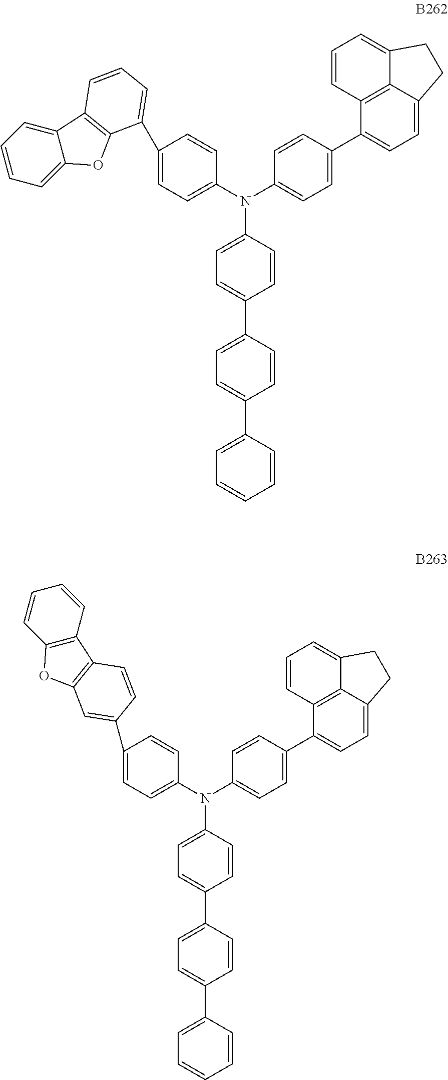 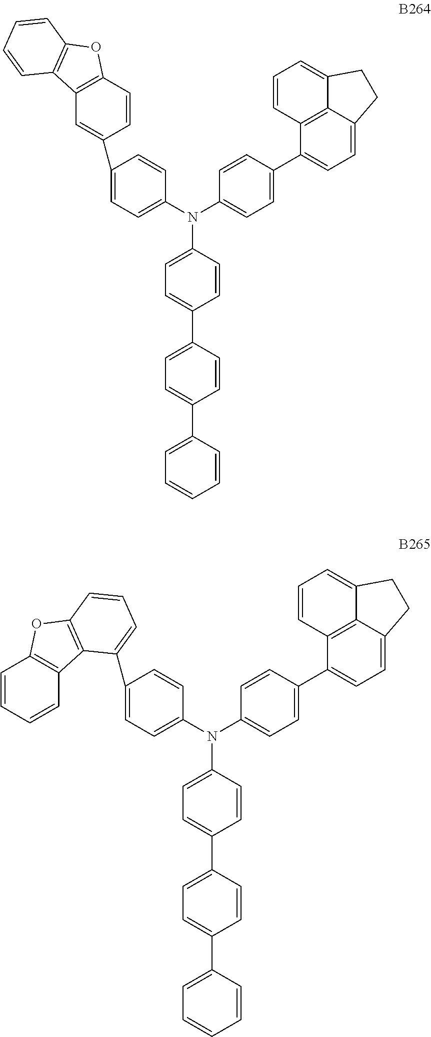 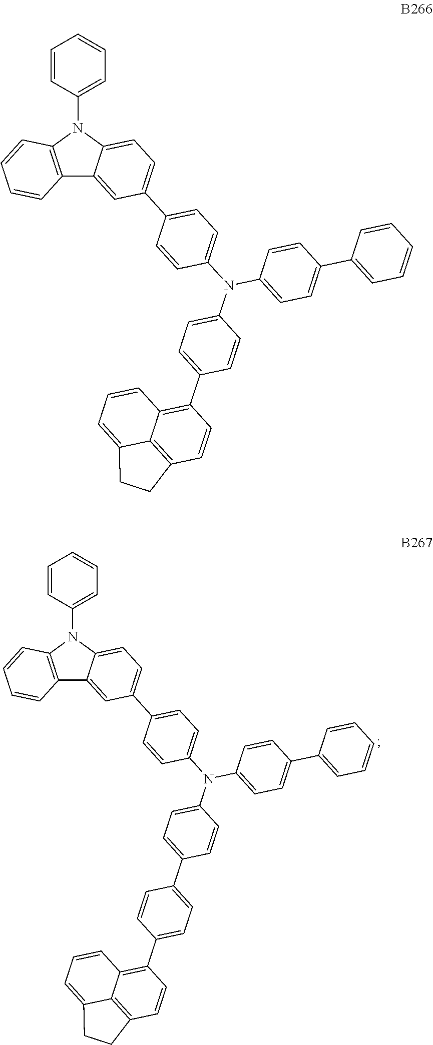
wherein, the substituted compound W4 is formed by replacing one or more hydrogens on the unsubstituted compound W4 with deuterium.

15. The aromatic amine compound according to claim 14, wherein, the substituted compound W4 is formed by replacing all hydrogens on SP²-hybridized carbons of the unsubstituted compound W4 with deuterium.

16. The aromatic amine compound according to claim 1, wherein a structural formula of the aromatic amine compounds represented by Chemical Formula II: 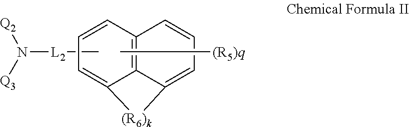
wherein, L₂ is selected from a single bond, a substituted or unsubstituted aryl group having 6 to 30 carbon atoms, or a substituted or unsubstituted heteroaryl group having 3 to 30 carbon atoms; Q₂ and Q₃ are each independently selected from a substituted or unsubstituted aryl group having 6 to 50 carbon atoms or a substituted or unsubstituted heteroaryl group having 3 to 50 carbon atoms, with at least one of Q₂ or Q₃ containing a fused heteroaromatic ring; the heteroatoms in the fused heteroaromatic ring are independently selected from N, O, and S.

17. The aromatic amine compound according to claim 16, wherein at least one of Q₂ or Q₃ contains the following fused heteroaromatic ring groups: 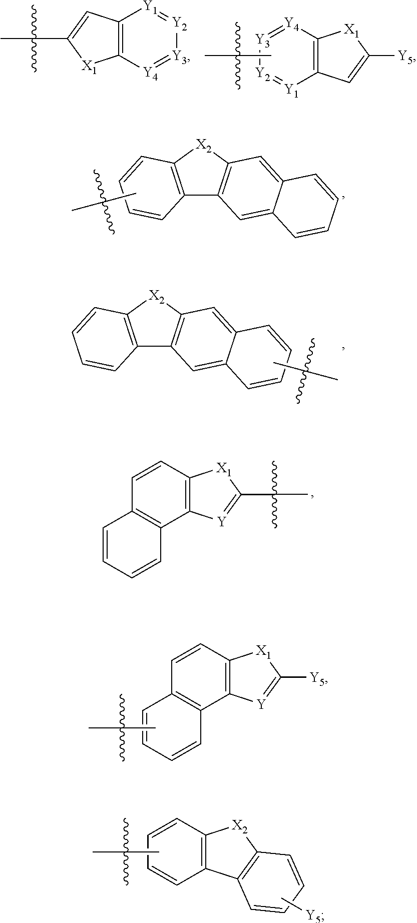
wherein, X₁ is selected from C(Y₆Y₇), NY₈, O, or S; X₂ is selected from NY₈, O, or S;
Y₁, Y₂, Y₃, and Y₄ are each independently selected from CY₈ or N, with only one of them being N;
Y₅, Y₆, Y₇, and Y₈ are each independently selected from hydrogen, deuterium, an alkyl group having 1 to 30 carbon atoms, an arylene group having 6 to 20 carbon atoms, or a heteroaryl group having 5 to 20 carbon atoms.

18. The aromatic amine compound according to claim 17, wherein X₁ is selected from O or S; X₂ is selected from O or S; Y₈ is selected from hydrogen or deuterium; Y₈ is selected from hydrogen, deuterium, phenyl, biphenyl, terphenyl, naphthyl, anthracenyl, or phenanthrenyl.

19. The aromatic amine compound according to claim 17, wherein at least one of Q₂ or Q₃ is selected from the group consisting of the following substituents:

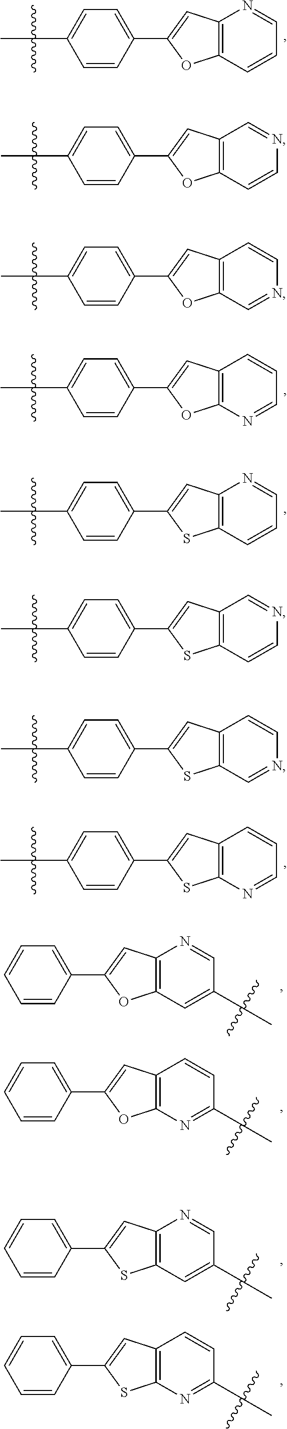

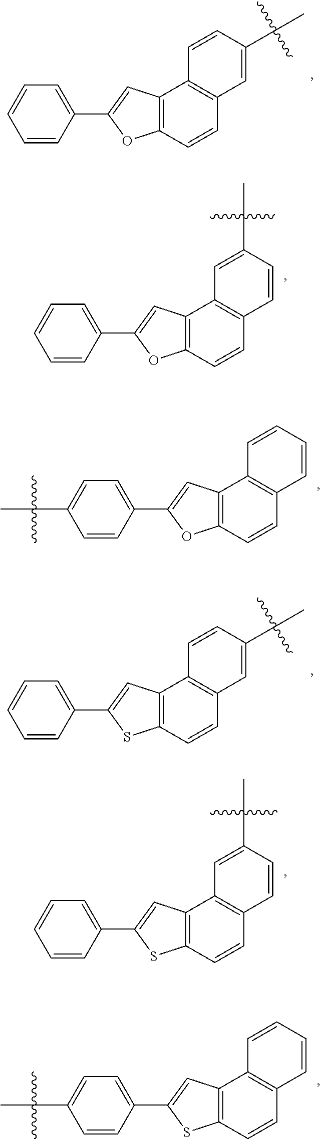 [SMILES: *c1ccc2c(ccc3oc(-c4ccccc4)cc32)c1 ; *c1ccc2ccc3oc(-c4ccccc4)cc3c2c1 ; *c1ccc(-c2cc3c(ccc4ccccc43)o2)cc1 ; *c1ccc2c(ccc3sc(-c4ccccc4)cc32)c1 ; *c1ccc2ccc3sc(-c4ccccc4)cc3c2c1 ; *c1ccc(-c2cc3c(ccc4ccccc43)s2)cc1]

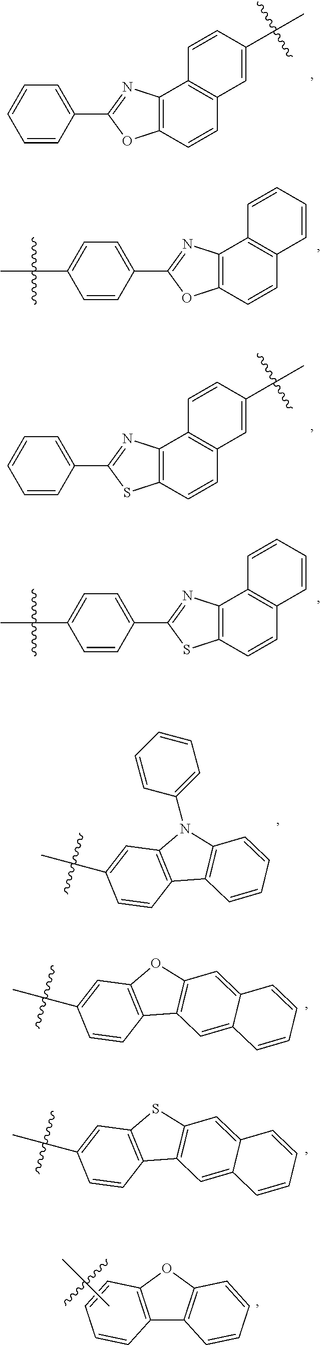

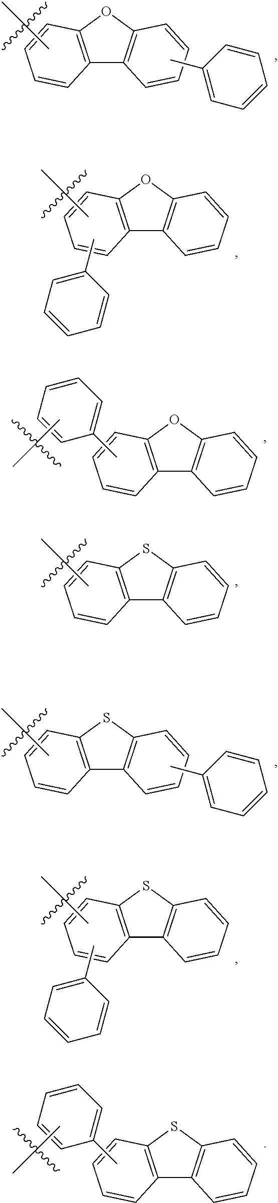

20. The aromatic amine compound according to claim 16, wherein when Q₂ does not contain a fused heteroaromatic ring, Q₂ is selected from a substituted or unsubstituted group W5; when Q₃ does not contain a fused heteroaromatic ring, Q₃ is selected from a substituted or unsubstituted group W5;
the unsubstituted group W5 is selected from an aryl group having 6 to 40 carbon atoms or a heteroaryl group having 5 to 40 carbon atoms;
wherein, the substituted group W5 is formed by replacing one or more hydrogens on the unsubstituted group W5 with substituents selected from deuterium, fluorine, chlorine, bromine, an alkyl group having 1 to 6 carbon atoms, a cycloalkyl group having 6 to 10 carbon atoms, or an aryl group having 6 to 12 carbon atoms; when the number of substituents on L₂ is plural, any two substituents are the same or different.

21. The aromatic amine compound according to claim 20, wherein the unsubstituted group W5 is selected from biphenyl, terphenyl, naphthyl, naphthyl-substituted phenyl, naphthyl-substituted biphenyl, anthracenyl, anthracenyl-substituted phenyl, anthracenyl-substituted biphenyl, phenanthrenyl, phenanthrenyl-substituted phenyl, or phenanthrenyl-substituted biphenyl.

22. The aromatic amine compound according to claim 16, wherein L₂ is selected from phenylene, biphenylene, terphenylene, naphthylene, dibenzofuranylene, or dibenzothienylene.

23. The aromatic amine compound according to claim 16, wherein the aromatic amine compounds are selected from the group consisting of the following compounds:

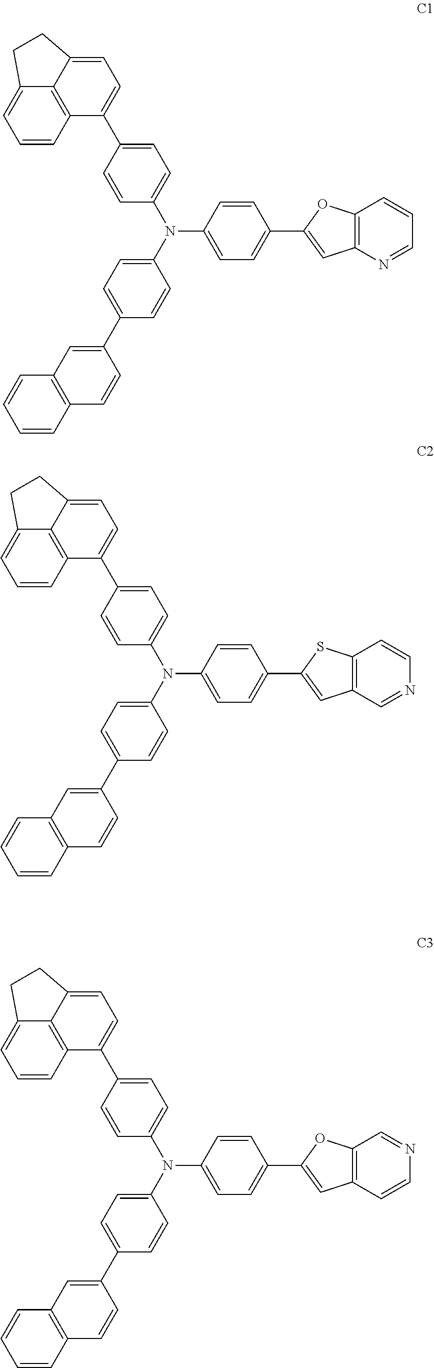

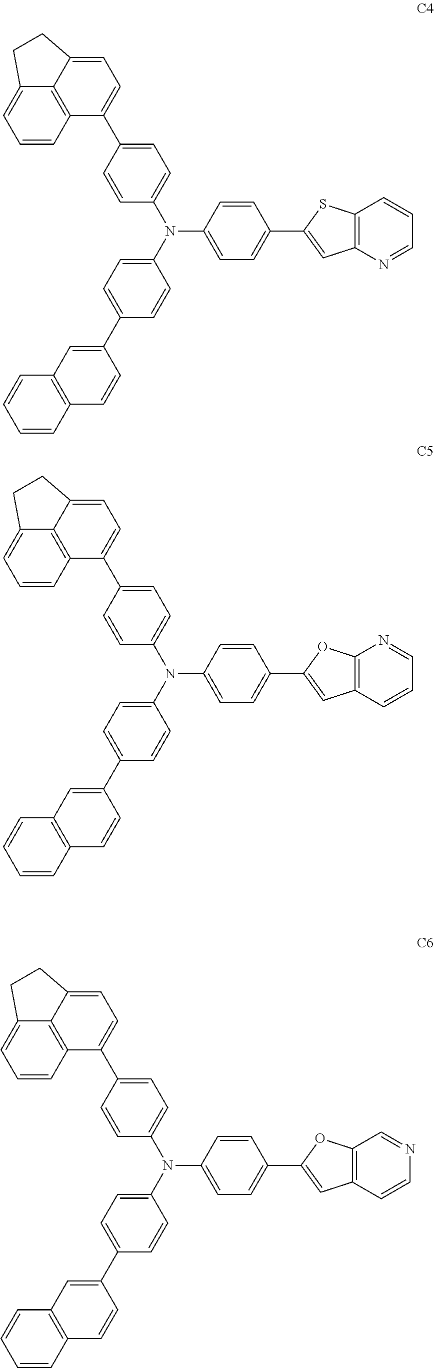

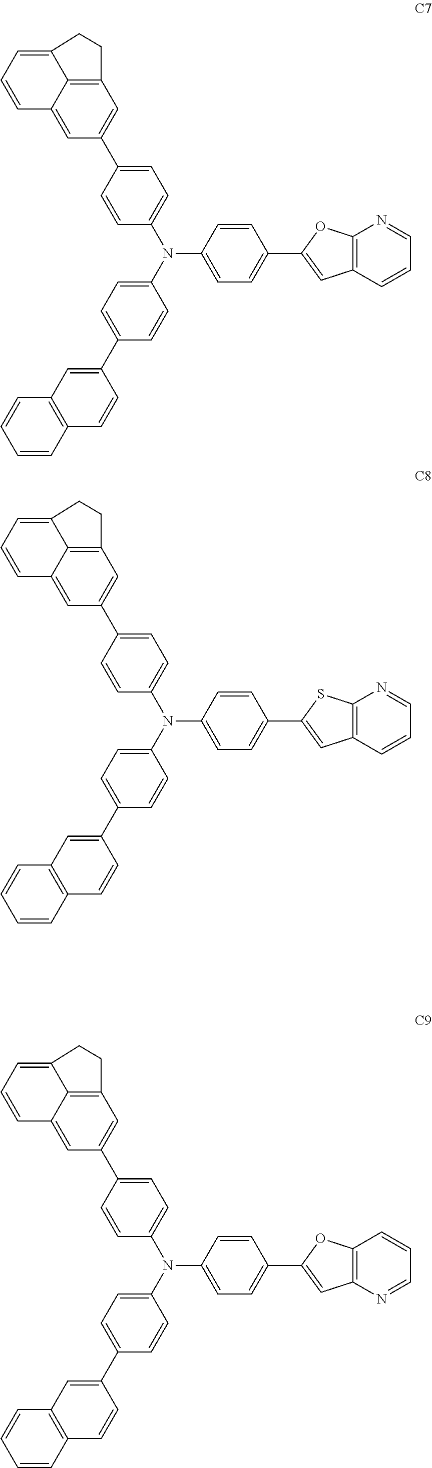 [SMILES: c1ccc2cc(-c3ccc(N(c4ccc(-c5cc6c7c(cccc7c5)CC6)cc4)c4ccc(-c5cc6cccnc6o5)cc4)cc3)ccc2c1 ; c1ccc2cc(-c3ccc(N(c4ccc(-c5cc6c7c(cccc7c5)CC6)cc4)c4ccc(-c5cc6cccnc6s5)cc4)cc3)ccc2c1 ; c1ccc2cc(-c3ccc(N(c4ccc(-c5cc6c7c(cccc7c5)CC6)cc4)c4ccc(-c5cc6ncccc6o5)cc4)cc3)ccc2c1]

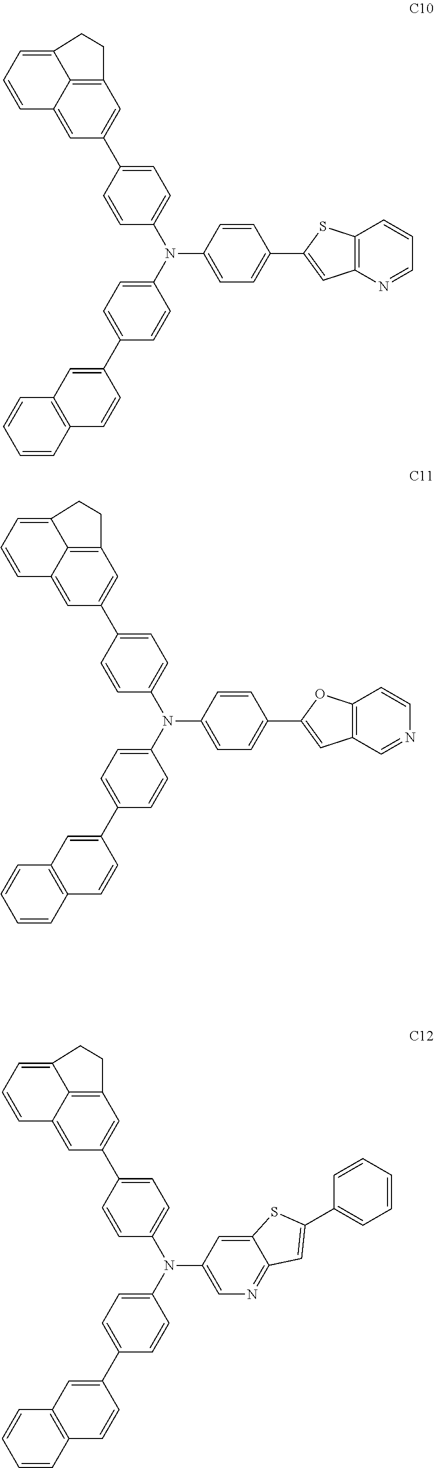 [SMILES: c1ccc2cc(-c3ccc(N(c4ccc(-c5cc6c7c(cccc7c5)CC6)cc4)c4ccc(-c5cc6ncccc6s5)cc4)cc3)ccc2c1 ; c1ccc2cc(-c3ccc(N(c4ccc(-c5cc6c7c(cccc7c5)CC6)cc4)c4ccc(-c5cc6cnccc6o5)cc4)cc3)ccc2c1 ; c1ccc(-c2cc3ncc(N(c4ccc(-c5ccc6ccccc6c5)cc4)c4ccc(-c5cc6c7c(cccc7c5)CC6)cc4)cc3s2)cc1]

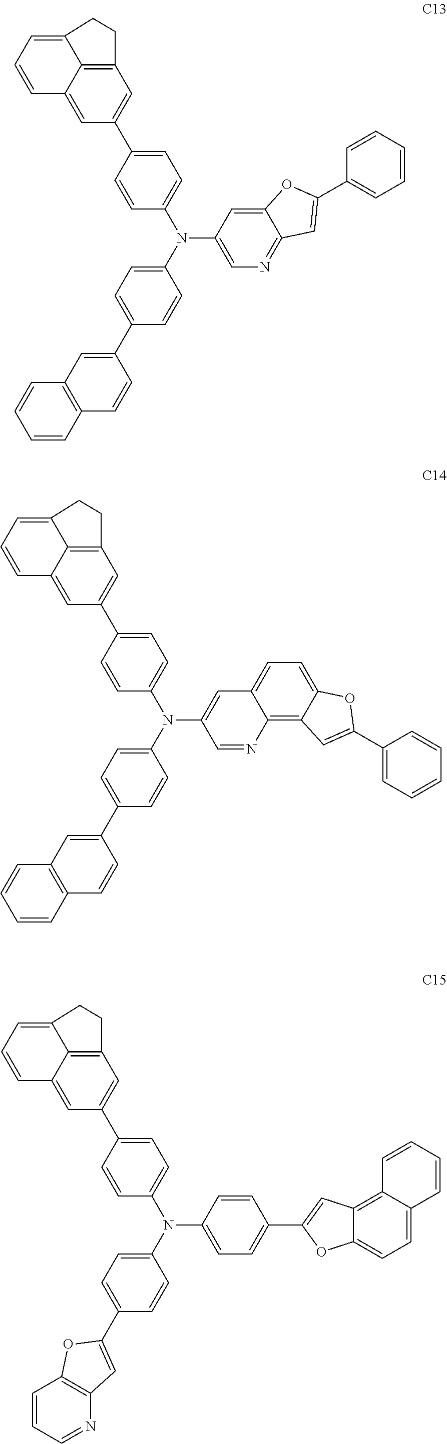 [SMILES: c1ccc(-c2cc3ncc(N(c4ccc(-c5ccc6ccccc6c5)cc4)c4ccc(-c5cc6c7c(cccc7c5)CC6)cc4)cc3o2)cc1 ; c1ccc(-c2cc3c(ccc4cc(N(c5ccc(-c6ccc7ccccc7c6)cc5)c5ccc(-c6cc7c8c(cccc8c6)CC7)cc5)cnc43)o2)cc1 ; c1cc2c3c(cc(-c4ccc(N(c5ccc(-c6cc7ncccc7o6)cc5)c5ccc(-c6cc7c(ccc8ccccc87)o6)cc5)cc4)cc3c1)CC2]

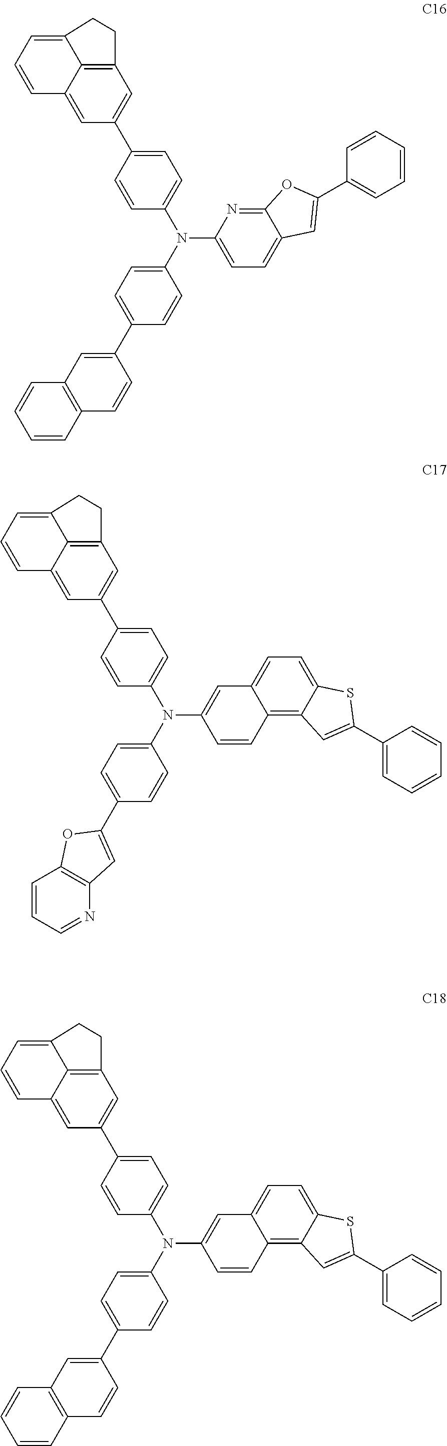 [SMILES: c1ccc(-c2cc3ccc(N(c4ccc(-c5ccc6ccccc6c5)cc4)c4ccc(-c5cc6c7c(cccc7c5)CC6)cc4)nc3o2)cc1 ; c1ccc(-c2cc3c(ccc4cc(N(c5ccc(-c6cc7c8c(cccc8c6)CC7)cc5)c5ccc(-c6cc7ncccc7o6)cc5)ccc43)s2)cc1 ; c1ccc(-c2cc3c(ccc4cc(N(c5ccc(-c6ccc7ccccc7c6)cc5)c5ccc(-c6cc7c8c(cccc8c6)CC7)cc5)ccc43)s2)cc1]

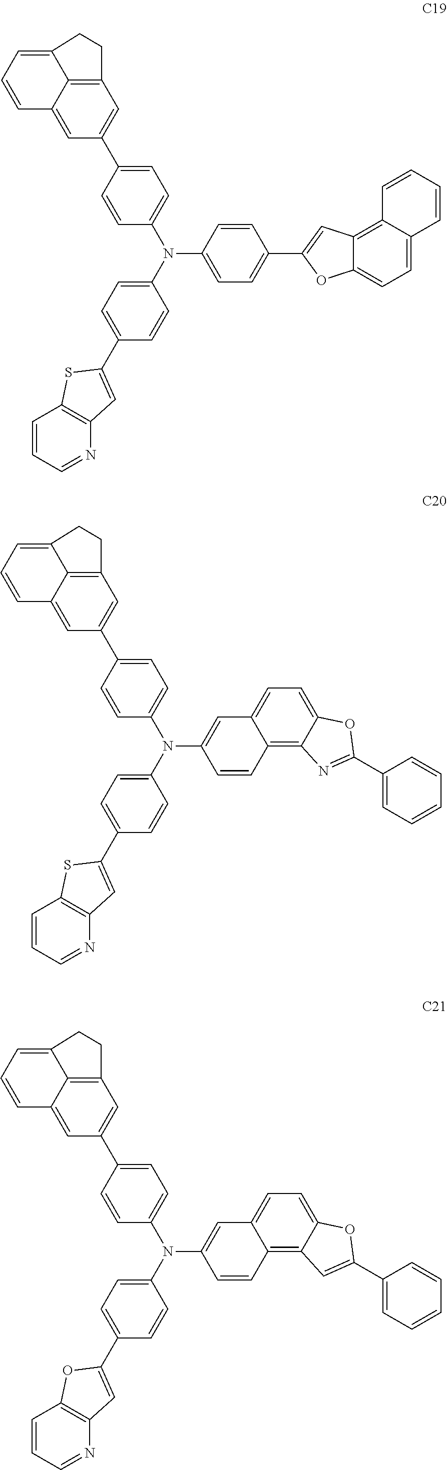 [SMILES: c1cc2c3c(cc(-c4ccc(N(c5ccc(-c6cc7c(ccc8ccccc87)o6)cc5)c5ccc(-c6cc7ncccc7s6)cc5)cc4)cc3c1)CC2 ; c1ccc(-c2nc3c(ccc4cc(N(c5ccc(-c6cc7c8c(cccc8c6)CC7)cc5)c5ccc(-c6cc7ncccc7s6)cc5)ccc43)o2)cc1 ; c1ccc(-c2cc3c(ccc4cc(N(c5ccc(-c6cc7c8c(cccc8c6)CC7)cc5)c5ccc(-c6cc7ncccc7o6)cc5)ccc43)o2)cc1]

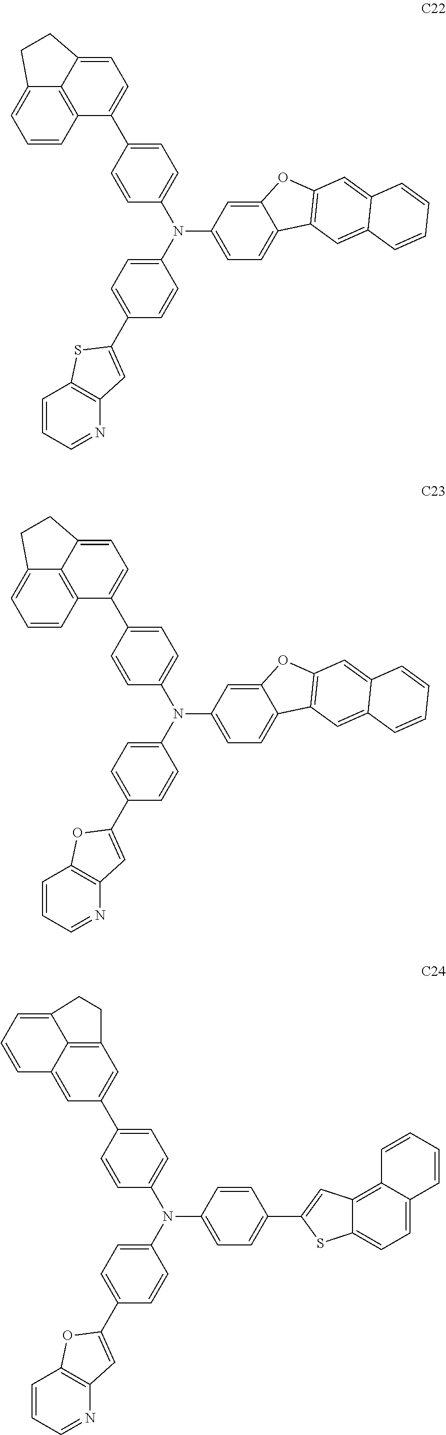 [SMILES: c1ccc2cc3c(cc2c1)oc1cc(N(c2ccc(-c4cc5ncccc5s4)cc2)c2ccc(-c4ccc5c6c(cccc46)CC5)cc2)ccc13 ; c1ccc2cc3c(cc2c1)oc1cc(N(c2ccc(-c4cc5ncccc5o4)cc2)c2ccc(-c4ccc5c6c(cccc46)CC5)cc2)ccc13 ; c1cc2c3c(cc(-c4ccc(N(c5ccc(-c6cc7ncccc7o6)cc5)c5ccc(-c6cc7c(ccc8ccccc87)s6)cc5)cc4)cc3c1)CC2]

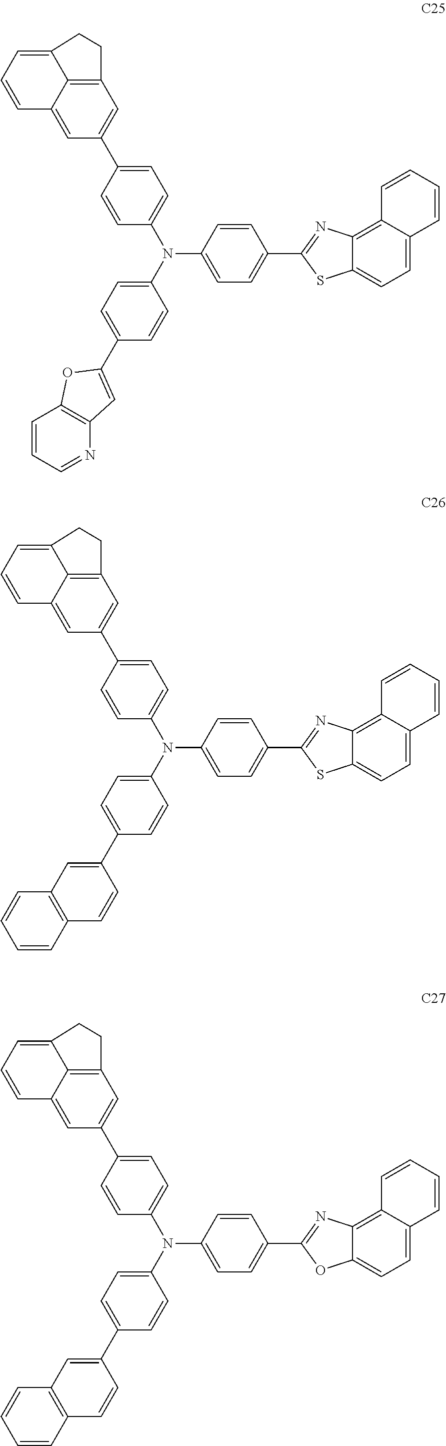 [SMILES: c1cc2c3c(cc(-c4ccc(N(c5ccc(-c6cc7ncccc7o6)cc5)c5ccc(-c6nc7c(ccc8ccccc87)s6)cc5)cc4)cc3c1)CC2 ; c1ccc2cc(-c3ccc(N(c4ccc(-c5cc6c7c(cccc7c5)CC6)cc4)c4ccc(-c5nc6c(ccc7ccccc76)s5)cc4)cc3)ccc2c1 ; c1ccc2cc(-c3ccc(N(c4ccc(-c5cc6c7c(cccc7c5)CC6)cc4)c4ccc(-c5nc6c(ccc7ccccc76)o5)cc4)cc3)ccc2c1]

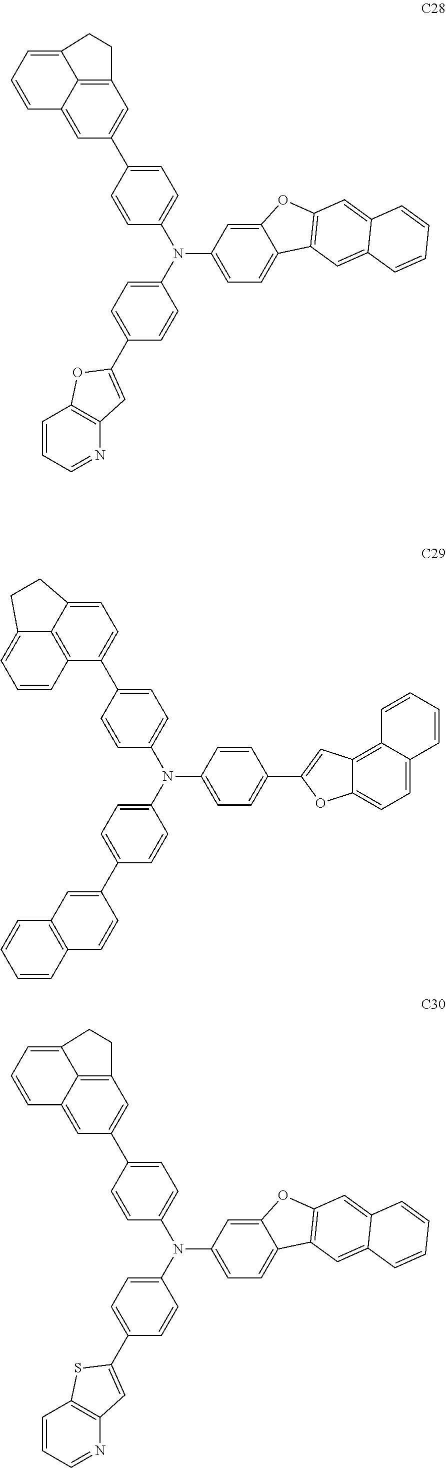 [SMILES: c1ccc2cc3c(cc2c1)oc1cc(N(c2ccc(-c4cc5c6c(cccc6c4)CC5)cc2)c2ccc(-c4cc5ncccc5o4)cc2)ccc13 ; c1ccc2cc(-c3ccc(N(c4ccc(-c5cc6c(ccc7ccccc76)o5)cc4)c4ccc(-c5ccc6c7c(cccc57)CC6)cc4)cc3)ccc2c1 ; c1ccc2cc3c(cc2c1)oc1cc(N(c2ccc(-c4cc5c6c(cccc6c4)CC5)cc2)c2ccc(-c4cc5ncccc5s4)cc2)ccc13]

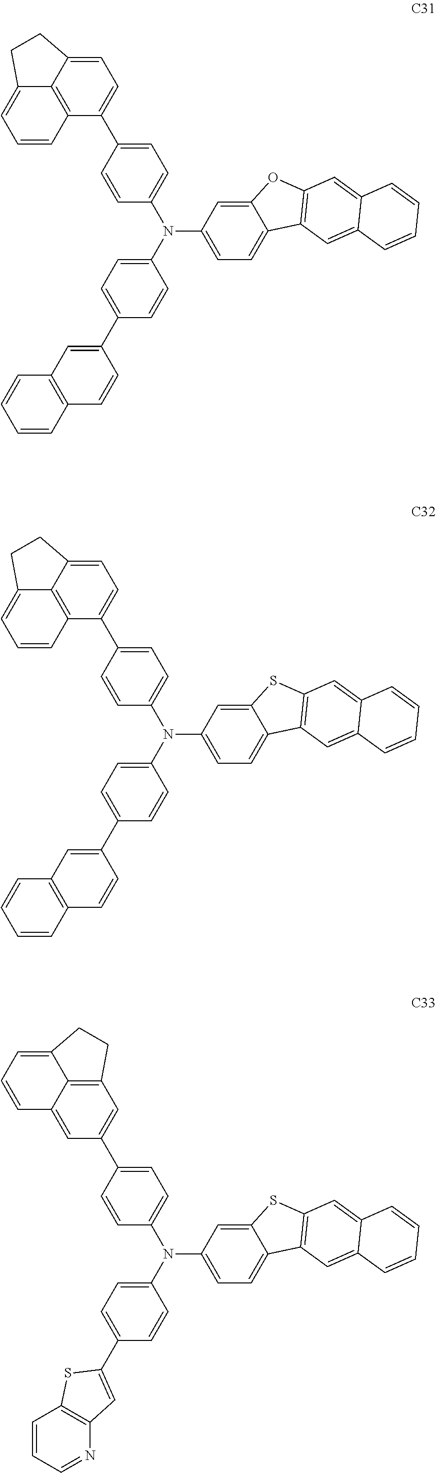 [SMILES: c1ccc2cc(-c3ccc(N(c4ccc(-c5ccc6c7c(cccc57)CC6)cc4)c4ccc5c(c4)oc4cc6ccccc6cc45)cc3)ccc2c1 ; c1ccc2cc(-c3ccc(N(c4ccc(-c5ccc6c7c(cccc57)CC6)cc4)c4ccc5c(c4)sc4cc6ccccc6cc45)cc3)ccc2c1 ; c1ccc2cc3c(cc2c1)sc1cc(N(c2ccc(-c4cc5c6c(cccc6c4)CC5)cc2)c2ccc(-c4cc5ncccc5s4)cc2)ccc13]

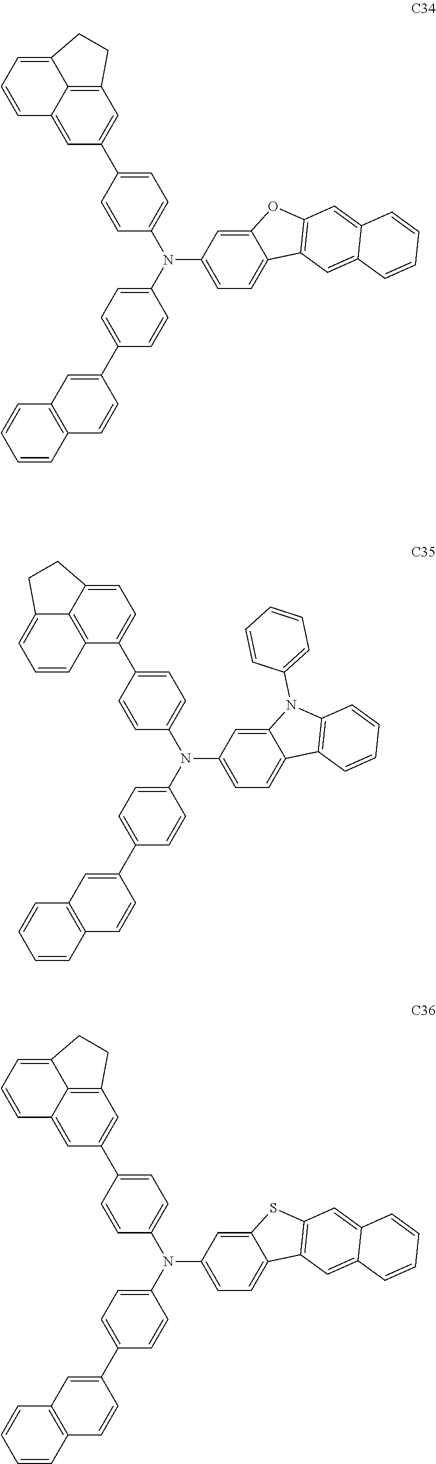 [SMILES: c1ccc2cc(-c3ccc(N(c4ccc(-c5cc6c7c(cccc7c5)CC6)cc4)c4ccc5c(c4)oc4cc6ccccc6cc45)cc3)ccc2c1 ; c1ccc(-n2c3ccccc3c3ccc(N(c4ccc(-c5ccc6ccccc6c5)cc4)c4ccc(-c5ccc6c7c(cccc57)CC6)cc4)cc32)cc1 ; c1ccc2cc(-c3ccc(N(c4ccc(-c5cc6c7c(cccc7c5)CC6)cc4)c4ccc5c(c4)sc4cc6ccccc6cc45)cc3)ccc2c1]

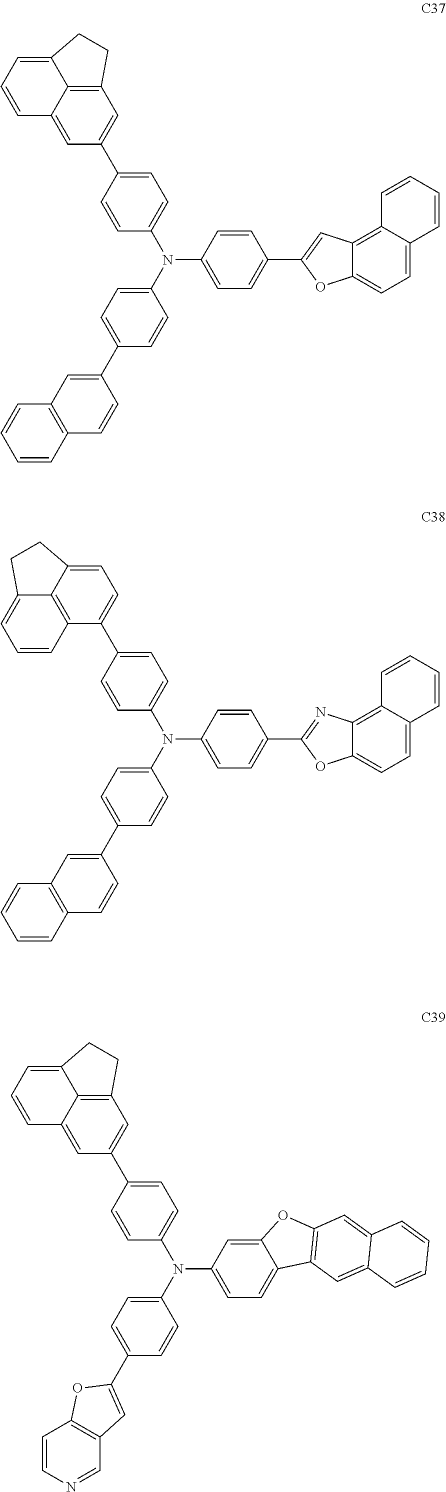 [SMILES: c1ccc2cc(-c3ccc(N(c4ccc(-c5cc6c7c(cccc7c5)CC6)cc4)c4ccc(-c5cc6c(ccc7ccccc76)o5)cc4)cc3)ccc2c1 ; c1ccc2cc(-c3ccc(N(c4ccc(-c5nc6c(ccc7ccccc76)o5)cc4)c4ccc(-c5ccc6c7c(cccc57)CC6)cc4)cc3)ccc2c1 ; c1ccc2cc3c(cc2c1)oc1cc(N(c2ccc(-c4cc5c6c(cccc6c4)CC5)cc2)c2ccc(-c4cc5cnccc5o4)cc2)ccc13]

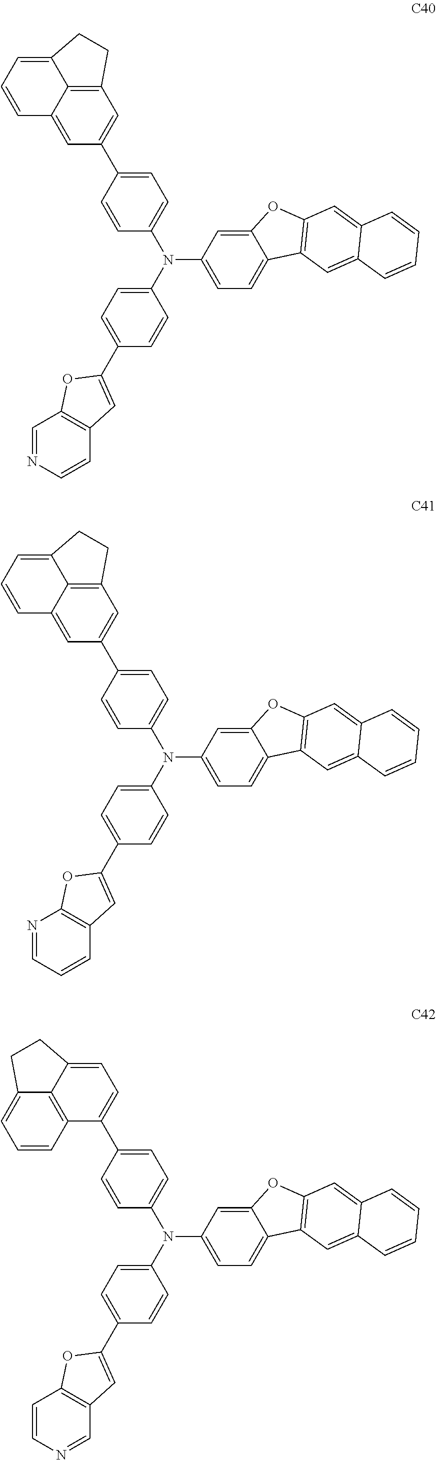 [SMILES: c1ccc2cc3c(cc2c1)oc1cc(N(c2ccc(-c4cc5c6c(cccc6c4)CC5)cc2)c2ccc(-c4cc5ccncc5o4)cc2)ccc13 ; c1ccc2cc3c(cc2c1)oc1cc(N(c2ccc(-c4cc5c6c(cccc6c4)CC5)cc2)c2ccc(-c4cc5cccnc5o4)cc2)ccc13 ; c1ccc2cc3c(cc2c1)oc1cc(N(c2ccc(-c4cc5cnccc5o4)cc2)c2ccc(-c4ccc5c6c(cccc46)CC5)cc2)ccc13]

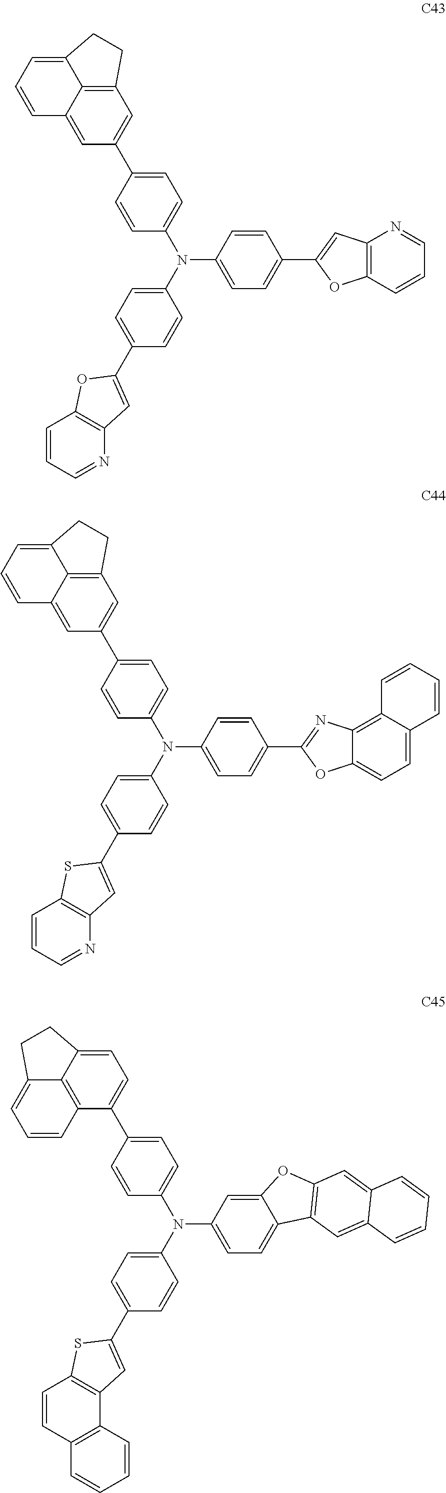

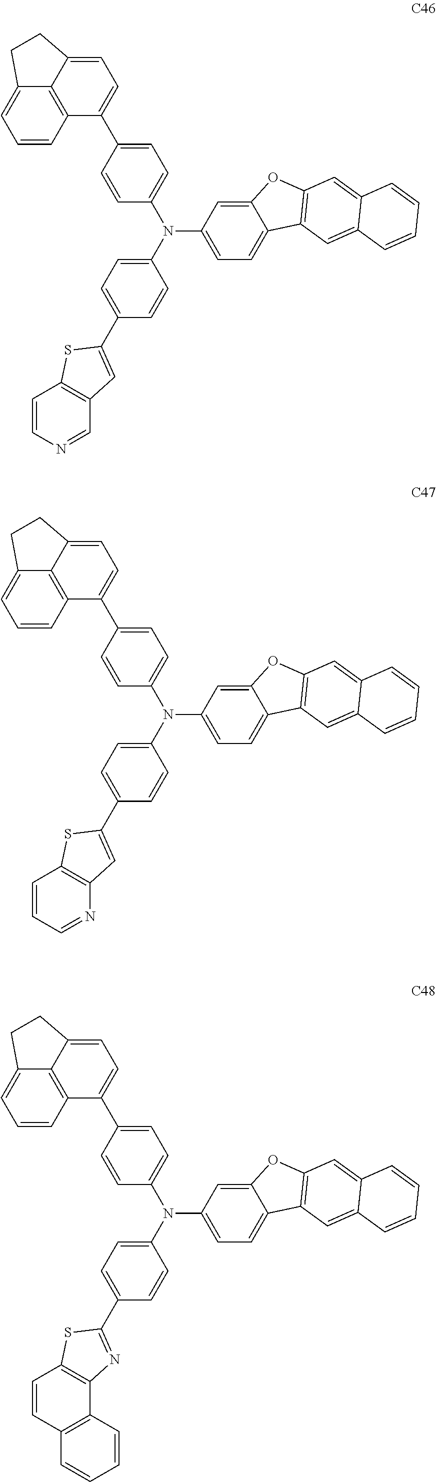 [SMILES: c1ccc2cc3c(cc2c1)oc1cc(N(c2ccc(-c4cc5cnccc5s4)cc2)c2ccc(-c4ccc5c6c(cccc46)CC5)cc2)ccc13 ; c1ccc2cc3c(cc2c1)oc1cc(N(c2ccc(-c4cc5ncccc5s4)cc2)c2ccc(-c4ccc5c6c(cccc46)CC5)cc2)ccc13 ; c1ccc2cc3c(cc2c1)oc1cc(N(c2ccc(-c4nc5c(ccc6ccccc65)s4)cc2)c2ccc(-c4ccc5c6c(cccc46)CC5)cc2)ccc13]

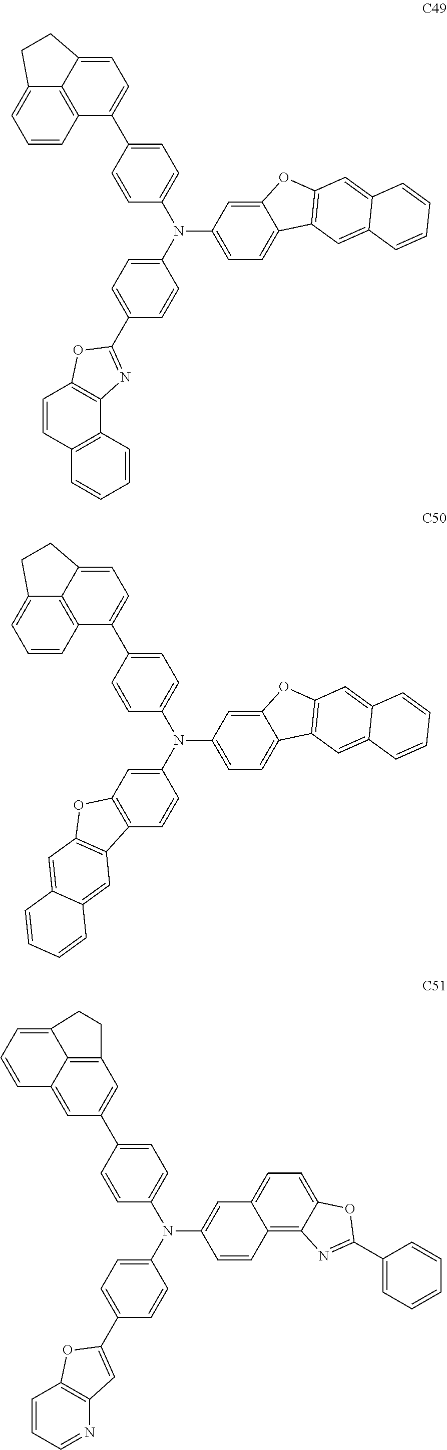 [SMILES: c1ccc2cc3c(cc2c1)oc1cc(N(c2ccc(-c4nc5c(ccc6ccccc65)o4)cc2)c2ccc(-c4ccc5c6c(cccc46)CC5)cc2)ccc13 ; c1ccc2cc3c(cc2c1)oc1cc(N(c2ccc(-c4ccc5c6c(cccc46)CC5)cc2)c2ccc4c(c2)oc2cc5ccccc5cc24)ccc13 ; c1ccc(-c2nc3c(ccc4cc(N(c5ccc(-c6cc7c8c(cccc8c6)CC7)cc5)c5ccc(-c6cc7ncccc7o6)cc5)ccc43)o2)cc1]

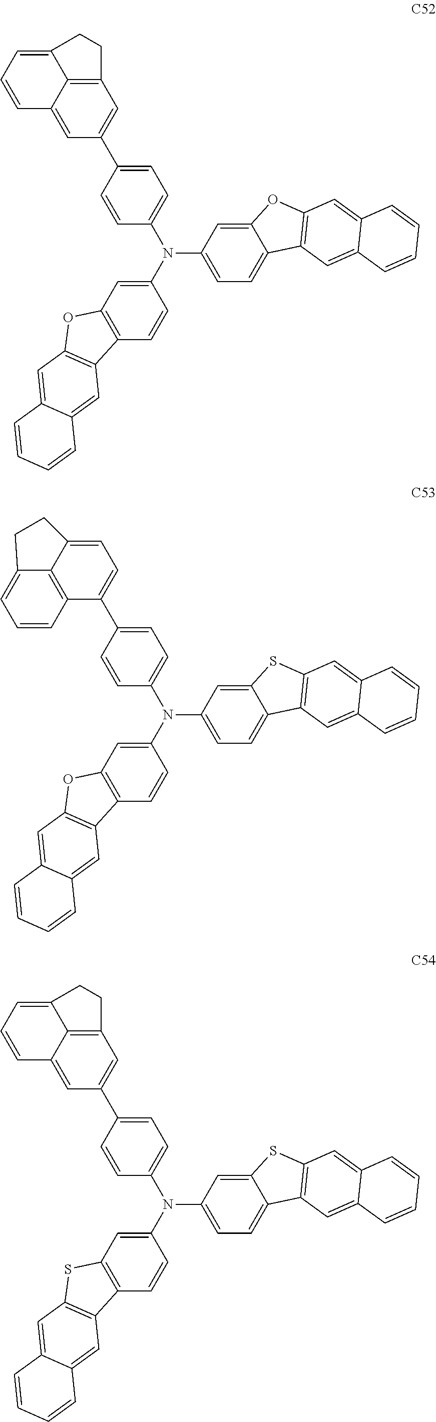 [SMILES: c1ccc2cc3c(cc2c1)oc1cc(N(c2ccc(-c4cc5c6c(cccc6c4)CC5)cc2)c2ccc4c(c2)oc2cc5ccccc5cc24)ccc13 ; c1ccc2cc3c(cc2c1)oc1cc(N(c2ccc(-c4ccc5c6c(cccc46)CC5)cc2)c2ccc4c(c2)sc2cc5ccccc5cc24)ccc13 ; c1ccc2cc3c(cc2c1)sc1cc(N(c2ccc(-c4cc5c6c(cccc6c4)CC5)cc2)c2ccc4c(c2)sc2cc5ccccc5cc24)ccc13]

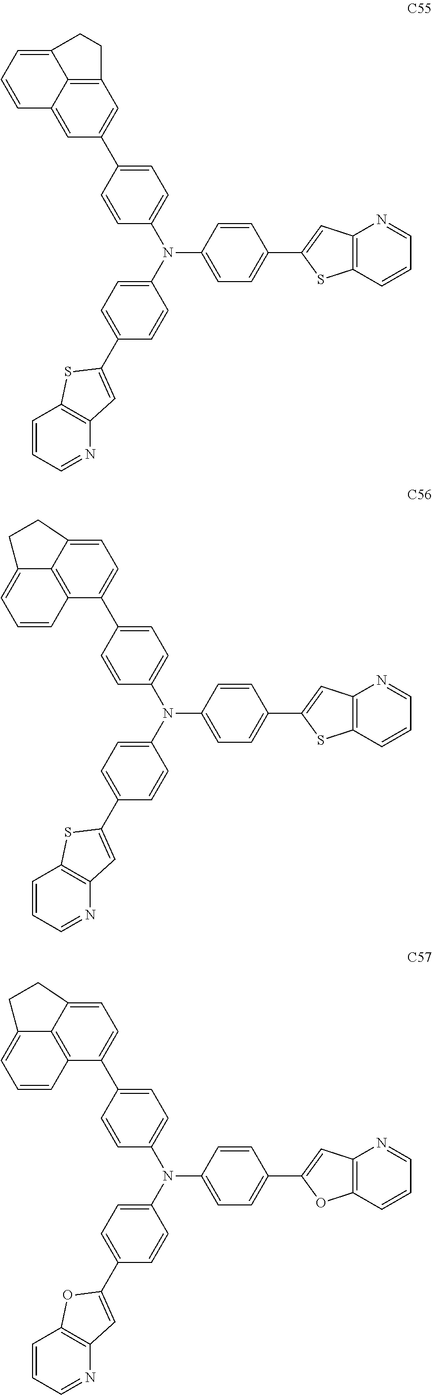 [SMILES: c1cc2c3c(cc(-c4ccc(N(c5ccc(-c6cc7ncccc7s6)cc5)c5ccc(-c6cc7ncccc7s6)cc5)cc4)cc3c1)CC2 ; c1cnc2cc(-c3ccc(N(c4ccc(-c5cc6ncccc6s5)cc4)c4ccc(-c5ccc6c7c(cccc57)CC6)cc4)cc3)sc2c1 ; c1cnc2cc(-c3ccc(N(c4ccc(-c5cc6ncccc6o5)cc4)c4ccc(-c5ccc6c7c(cccc57)CC6)cc4)cc3)oc2c1]

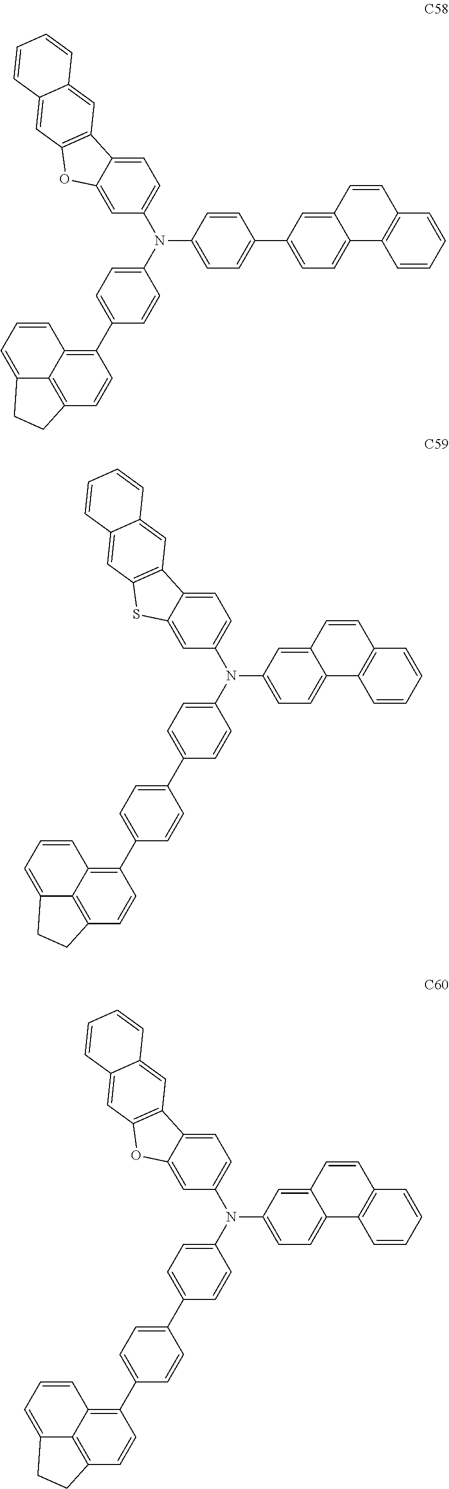 [SMILES: c1ccc2cc3c(cc2c1)oc1cc(N(c2ccc(-c4ccc5c(ccc6ccccc65)c4)cc2)c2ccc(-c4ccc5c6c(cccc46)CC5)cc2)ccc13 ; c1ccc2cc3c(cc2c1)sc1cc(N(c2ccc(-c4ccc(-c5ccc6c7c(cccc57)CC6)cc4)cc2)c2ccc4c(ccc5ccccc54)c2)ccc13 ; c1ccc2cc3c(cc2c1)oc1cc(N(c2ccc(-c4ccc(-c5ccc6c7c(cccc57)CC6)cc4)cc2)c2ccc4c(ccc5ccccc54)c2)ccc13]

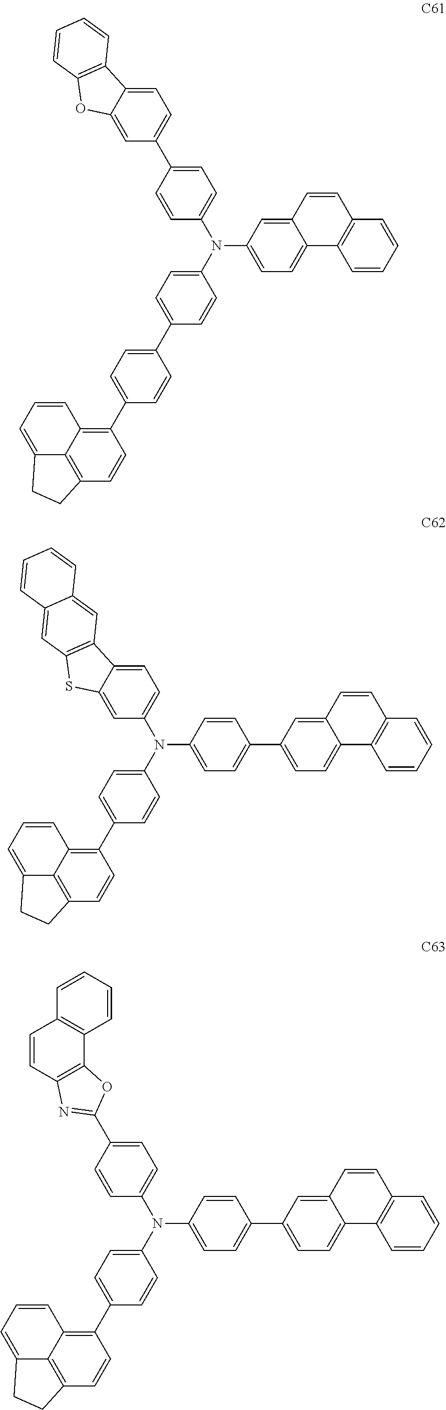 [SMILES: c1ccc2c(c1)ccc1cc(N(c3ccc(-c4ccc(-c5ccc6c7c(cccc57)CC6)cc4)cc3)c3ccc(-c4ccc5c(c4)oc4ccccc45)cc3)ccc12 ; c1ccc2cc3c(cc2c1)sc1cc(N(c2ccc(-c4ccc5c(ccc6ccccc65)c4)cc2)c2ccc(-c4ccc5c6c(cccc46)CC5)cc2)ccc13 ; c1ccc2c(c1)ccc1cc(-c3ccc(N(c4ccc(-c5nc6ccc7ccccc7c6o5)cc4)c4ccc(-c5ccc6c7c(cccc57)CC6)cc4)cc3)ccc12]

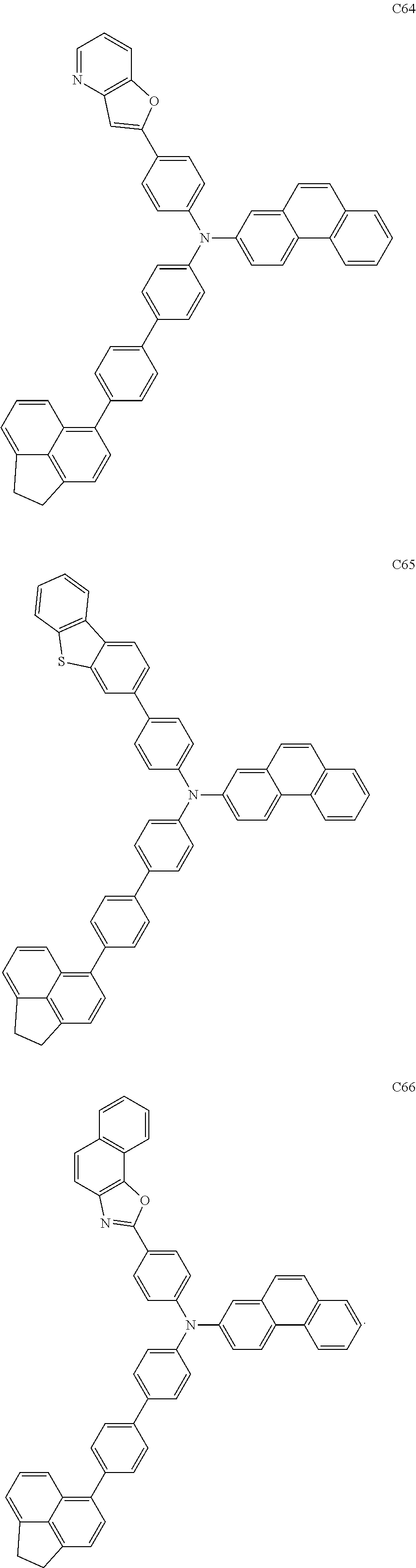 [SMILES: c1ccc2c(c1)ccc1cc(N(c3ccc(-c4ccc(-c5ccc6c7c(cccc57)CC6)cc4)cc3)c3ccc(-c4cc5ncccc5o4)cc3)ccc12 ; c1ccc2c(c1)ccc1cc(N(c3ccc(-c4ccc(-c5ccc6c7c(cccc57)CC6)cc4)cc3)c3ccc(-c4ccc5c(c4)sc4ccccc45)cc3)ccc12 ; c1ccc2c(c1)ccc1cc(N(c3ccc(-c4ccc(-c5ccc6c7c(cccc57)CC6)cc4)cc3)c3ccc(-c4nc5ccc6ccccc6c5o4)cc3)ccc12]

24. An electroluminescent device, wherein the electroluminescent device comprises an organic layer, and the organic layer comprises the aromatic amine compound;
wherein a structural formula of the aromatic amine compound represented by Chemical Formula A or Chemical Formula B: 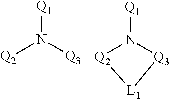
Chemical Formula A Chemical Formula B
wherein L₁ is selected from a single bond, a substituted or unsubstituted alkyl group with 1~10 carbon atoms, a substituted or unsubstituted cycloalkyl group with 1~10 carbon atoms, a substituted or unsubstituted aryl group with 6~30 carbon atoms, or a substituted or unsubstituted heteroaryl group with 3~30 carbon atoms;
Q₁, Q₂, and Q₃ are each independently selected from a substituted or unsubstituted aryl group with 6~50 carbon atoms or a substituted or unsubstituted heteroaryl group with 3~50 carbon atoms, and at least one of Q₁, Q₂, or Q₃ contains at least one group represented by Chemical Formula P1 to Chemical Formula P6: 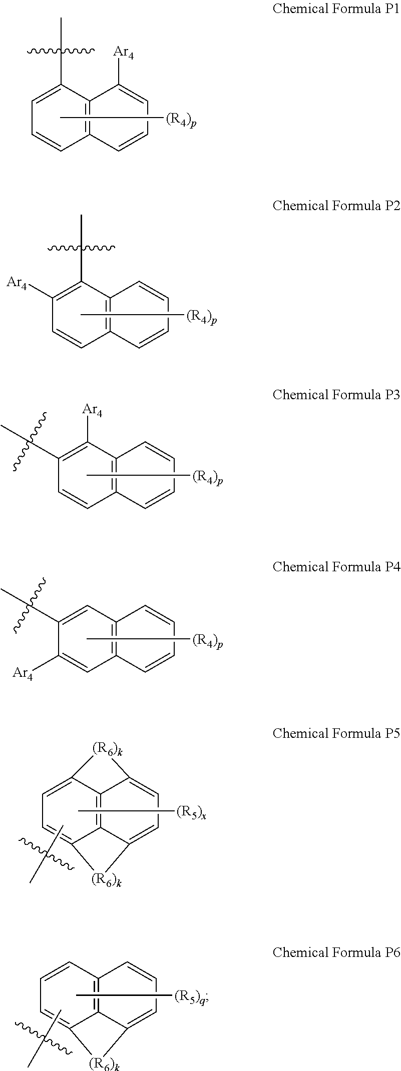
wherein Ar₄ is selected from hydrogen, deuterium, a substituted or unsubstituted alkyl group with 1~10 carbon atoms, a substituted or unsubstituted cycloalkyl group with 3~10 carbon atoms, a substituted or unsubstituted aryl group with 630 carbon atoms, a substituted or unsubstituted heteroaryl group with 3~30 carbon atoms or a substituted or unsubstituted cycloalkane-naphthyl group with 14~30 carbon atoms;
R₄ and R₅ are the same or different, each independently selected from deuterium, a substituted or unsubstituted alkyl group with 1~20 carbon atoms, a substituted or unsubstituted cycloalkyl group with 3~10 carbon atoms a substituted or unsubstituted aryl group with 6~20 carbon atoms, a substituted or unsubstituted heteroaryl group with 3~20 carbon atoms, a substituted or unsubstituted silyl group, or a substituted or unsubstituted arylsilyl group with 6~20 carbon atoms;
R₆ is C(R₇R₈); R₇ and R₈ are each independently selected from hydrogen, deuterium, fluorine, an alkyl group with 1~4 carbon atoms, or a substituted or unsubstituted aryl group with 6~12 carbon atoms;
p represents an integer from 0 to 6 when p is greater than 1, any two R₄ groups are the same or different;
q represents an integer from 0 to 4; when q is greater than 1, any two R₅ groups are the same or different;
x represents an integer from 0 to 3; when x is greater than 1, any two R₅ groups are the same or different;
k is an integer from 2 to 4; any two R₇ groups are the same or different; any two R₈ groups are the same or different;
when L₁, Q₁, Q₂, R₄, R₅, R₇, R₈, and Ar₄ have substituents, these substituents may be the same or different and are each independently selected from deuterium halogen, an alkylsilyl group with 1~30 carbon atoms, an arylsilyl group with 6~30 carbon atoms, an arylamino group with 6~30 carbon atoms, an alkyl group with 1~30 carbon atoms, a cycloalkyl group with 5~10 carbon atoms, an alkenyl group with 2~30 carbon atoms, an aryl group with 6-~30 carbon atoms, or a heteroaryl group with 3~30 carbon atoms; 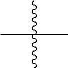
represents a connecting bond.

25.-30. (canceled)

31. A display apparatus, comprising the electroluminescent device, wherein the electroluminescent device comprises an aromatic amine compound;
wherein a structural formula of the aromatic amine compound represented by Chemical Formula A or Chemical Formula B; 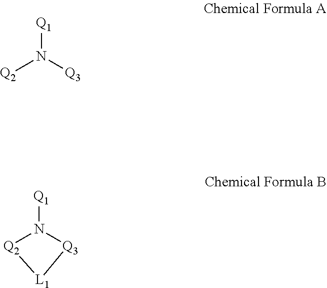
wherein L₁ is selected from a single bond, a substituted or unsubstituted alkyl group with 1~10 carbon atoms, a substituted or unsubstituted cycloalkyl group with 1~10 carbon atoms, a substituted or unsubstituted aryl group with 6~30 carbon atoms, or a substituted or unsubstituted heteroaryl group with 3~30 carbon atoms;
Q₁, Q₂, and Q₃ are each independently selected from a substituted or unsubstituted aryl group with 6~50 carbon atoms or a substituted or unsubstituted heteroaryl group with 3~50 carbon atoms and at least one of Q₁, Q₂, or Q₃ contains at least one group represented by Chemical Formula P1 to Chemical Formula P6: 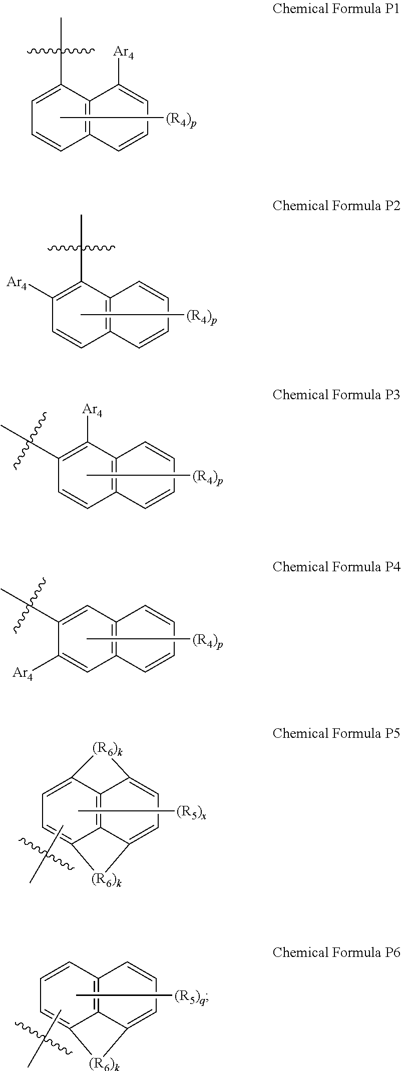
wherein Ar₄ is selected from hydrogen, deuterium, a substituted or unsubstituted alkyl group with 1~10 carbon atoms a substituted or unsubstituted cycloalkyl group with 3~10 carbon atoms, a substituted or unsubstituted aryl group with 6~30 carbon atoms, a substituted or unsubstituted heteroaryl group with 3~30 carbon atoms, or a substituted or unsubstituted cycloalkane-naphthyl group with 14~30 carbon atoms;
R₄ and R₅ are the same or different, each independently selected from deuterium, a substituted or unsubstituted alkyl group with 1~20 carbon atoms a substituted or unsubstituted cycloalkyl group with 3~10 carbon atoms, a substituted or unsubstituted aryl group with 6~20 carbon atoms, a substituted or unsubstituted heteroaryl group with 3~20 carbon atoms, a substituted or unsubstituted silyl group, or a substituted or unsubstituted arylsilyl group with 6~20 carbon atoms;
R₆ is C(R₇R₈); R₇ and R₈ are each independently selected from hydrogen, deuterium, fluorine, an alkyl group with 1-4 carbon atoms, or a substituted or unsubstituted aryl group with 6~12 carbon atoms;
p represents an integer from 0 to 6; when p is greater than 1, any two R₄ groups are the same or different;
q represents an integer from 0 to 4; when q is greater than 1 any two R₅ groups are the same or different;
x represents an integer from 0 to 3; when x is greater than 1, any two R₅ groups are the same or different;
k is an integer from 2 to 4; any two R₇ groups are the same or different; any two groups are the same or different;
when L₁, Q₁, Q₂, Q₃, R₄, R₅, R₇, R₈, and Ar₄ have substituents, these substituents may be the same or different and are each independently selected from deuterium, halogen, an alkylsilyl group with 1~30 carbon atoms, an arylsilyl group with 6~30 carbon atoms, an arylamino group with 6~30 carbon atoms, an alkyl group with 1~30 carbon atoms a cycloalkyl group with 5~10 carbon atoms, an alkenyl group with 2~30 carbon atoms, an aryl group with 6~30 carbon atoms, or a heteroaryl group with 3~30 carbon atoms; 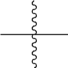
represents a connecting bond.
